(19) United States
(12) Patent Application Publication
(10) Pub. No.: US 2023/0073777 A1
(43) Pub. Date: Mar. 9, 2023

(54) CYCLIC-AMP RESPONSE ELEMENT BINDING PROTEIN (CBP) AND/OR ADENOVIRAL E1A BINDING PROTEIN OF 300 KDA (P300) DEGRADATION COMPOUNDS AND METHODS OF USE

(71) Applicant: CULLGEN (SHANGHAI), INC., Shanghai (CN)

(72) Inventors: Jing Liu, Oradell, NJ (US); Michael Bruno Plewe, San Diego, CA (US); Jialiang Wang, Nashville, TN (US); Xiaoran Han, Pudong New Area, Shanghai (CN); Liqun Chen, Shanghai (CN)

(21) Appl. No.: 17/434,587

(22) PCT Filed: Feb. 25, 2020
(86) PCT No.: PCT/CN2020/076648

§ 371 Date: Aug. 27, 2021

(30) Foreign Application Priority Data
Feb. 27, 2019 (CN) PCT/CN2019/076370
Oct. 31, 2019 (CN) PCT/CN2019/114835

(51) Int. Cl. A61K 47/55; A61K 47/54; A61P 35/00

(52) U.S. Cl. CPC A61K 47/55; A61P 35/00; A61K 47/545

(57) ABSTRACT

Bivalent compounds composition comprises one or more of the bivalent compounds. The bivalent compound comprises a cyclic-AMP response element binding protein (CBP) and/or adenoviral EA binding protein of 300 kDa (P300) ligand (CBP/P3000 ligand) conjugated to a degradation tag. The method of using the bivalent compounds is treating certain disease in a subject in need thereof. The method of identifying such bivalent compounds is disclosed.

### BACKGROUND OF THE INVENTION

This disclosure relates to bivalent compounds (e.g., bi-functional small molecule compounds), compositions comprising one or more of the bivalent compounds, and to methods of use of the bivalent compounds for the treatment of certain disease in a subject in need thereof. The disclosure also relates to methods for identifying such bivalent compounds.

### SUMMARY OF THE INVENTION

According to one aspect of the present disclosure, a bivalent compound disclosed herein comprises a cyclic-AMP response element binding protein (CBP) and/or adenoviral E1A binding protein of 300 kDa (P300) ligand conjugated to a degradation tag, or a pharmaceutically acceptable salt or analog thereof.

In one embodiment, the CBP/P300 ligand is capable of binding to a CBP/P300 protein comprising a CBP/P300, a CBP/P300 mutant, a CBP/P300 deletion, or a CBP/P300 fusion protein.

In one embodiment, the CBP/P300 ligand is a CBP/P300 inhibitor or a portion of CBP/P300 inhibitor.

In another embodiment, the CBP/P300 ligand is selected from the group consisting of GNE-781, GNE-272, GNE-207, CPD 4d, CPD (S)-8, CPD (R)-2, CPD 6, CPD 19, XDM-CBP, I-CBP112, TPOP146, CPI-637, SGC-CBP30, CPD 11, CPD 41, CPD 30, CPD 5, CPD 29, CPD 27, C646, A-485, naphthol-AS-E, MYBMIM, CCS1477, HBS1, OHM1, KCN1, ICG-001, YH249, YH250, and analogs thereof.

In another embodiment, the degradation tag binds to an ubiquitin ligase or is a hydrophobic group or a tag that leads to misfolding of the CBP/P300 protein.

In another embodiment, the ubiquitin ligase is an E3 ligase.

In another embodiment, the E3 ligase is selected from the group consisting of a cereblon E3 ligase, a VHL E3 ligase, an IAP ligase, a MDM2 ligase, a TRIM24 ligase, a TRIM21 ligase, a KEAP1 ligase, DCAF16 ligase, RNF4 ligase, RNF114 ligase, and AhR ligase.

In another embodiment, the degradation tag is selected from the group consisting of pomalidomide, thalidomide, lenalidomide, VHL-1, adamantane, 1-((4,4,5,5,5-pentafluoropentyl)sulfinyl)nonane, nutlin-3a, RG7112, RG7338, AMG232, AA-115, bestatin, MV-1, LCL161, CPD36, GDC-0152, CRBN-1, CRBN-2, CRBN-3, CRBN-4, CRBN-5, CRBN-6, CRBN-7, CRBN-8, CRBN-9, CRBN-10, CRBN-11, and analogs thereof.

In another embodiment, the CBP/P300 ligand is conjugated to the degradation tag via a linker moiety.

In another embodiment, the CBP/P300 ligand comprises a moiety of FORMULA 1:

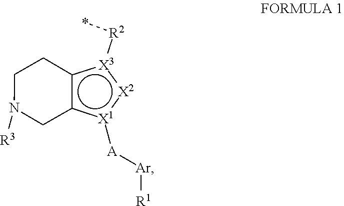

wherein

the linker moiety of the bivalent compound is attached to R²;

X¹ and X³ are independently selected from C and N, with the proviso that at least one of X¹ and X³ is C and at most only one of X¹ and X³ is N;

X² is selected from CR′, O, and NR′, wherein

R′ is selected from H, optionally substituted C₁-C8 alkyl, and optionally substituted 3-10 membered carbocyclyl;

A is selected from null, CR⁴R⁵, CO, O, S, SO, SO₂, and NR⁴, wherein

R⁴ and R⁵ are independently selected from hydrogen, halogen, hydroxyl, cyano, nitro, optionally substituted C₁-C₈ alkyl, optionally substituted C₂-C₈ alkenyl, optionally substituted C₂-C₈ alkynyl, optionally substituted C₁-C₈ alkoxy, optionally substituted C₁-C₈alkoxyC₁-C₈alkyl, optionally substituted C₁-C₈ alkylamino, optionally substituted C₁-C₈alkylaminoC₁-C₈alkyl, optionally substituted 3-10 membered carbocyclyl, optionally substituted 3-8 membered cycloalkoxy, optionally substituted 3-10 membered carbocyclylamino, optionally substituted 4-10 membered heterocyclyl, optionally substituted aryl, and optionally substituted heteroaryl;

Ar and R⁴, Ar and R⁵, and/or R⁴ and R⁵ together with the atom to which they are connected form an optionally substituted 3-20 membered cycloalkyl or heterocyclyl ring;

Ar is selected from aryl, heteroaryl, bicyclic aryl, bicyclic heteroaryl, tricyclic aryl, tricyclic heteroaryl groups, each of which is substituted with R¹ and optionally substituted with one or more substituents independently selected from hydrogen, halogen, oxo, CN, NO₂, OR⁶, SR⁶, NR⁶R⁷, OCOR⁶, OCO₂R⁶, OCONR⁶R⁷, COR⁶, CO₂R⁶, CONR⁶R⁷, SOR⁶, SO₂R⁶, SO₂NR⁶R⁷, NR⁸CO₂R⁶, NR⁸COR⁶, NR⁸C(O)NR⁶R⁷, NR⁸SOR⁶, NR⁸SO₂R⁶, NR⁸SO₂NR⁶R⁷, optionally substituted C₁-C₈ alkyl, optionally substituted C₂-C₈ alkenyl, optionally substituted C₂-C₈ alkynyl, optionally substituted C₁-C₈ alkoxy, optionally substituted C₁-C₈alkoxyC₁-C₈alkyl, optionally substituted C₁-C₈ alkylamino, optionally substituted C₁-C₈alkylaminoC₁-C₈alkyl, optionally substituted 3-10 membered carbocyclyl, optionally substituted 3-8 membered cycloalkoxy, optionally substituted 3-10 membered carbocyclylamino, optionally substituted 4-10 membered heterocyclyl, optionally substituted aryl, and optionally substituted heteroaryl, wherein

R⁶, R⁷, and R⁸ are independently selected from hydrogen, optionally substituted C₁-C₈ alkyl, optionally substituted C₂-C₈ alkenyl, optionally substituted C₂-C₈ alkynyl, optionally substituted C₁-C₈alkoxyC₁-C₈alkyl, optionally substituted C₁-C₈alkylaminoC₁-C₈alkyl, optionally substituted 3-10 membered carbocyclyl, optionally substituted 4-10 membered heterocyclyl, optionally substituted aryl, and optionally substituted heteroaryl, or

R⁶ and R⁷, R⁶ and R⁸ together with the atom to which they are connected form a 4-20 membered heterocyclyl ring;

R¹ is selected from hydrogen, halogen, CN, NO₂, OR⁹, SR⁹, NR⁹R¹⁰, OCOR⁹, OCO₂R⁹, OCONR⁹R¹⁰, COR⁹, CO₂R⁹, CONR⁹R¹⁰, SOR⁹, SO₂R⁹, SO₂NR⁹R¹⁰, NR¹¹CO₂R⁹, NR¹¹COR⁹, NR¹¹C(O)NR⁹R¹⁰, NR¹¹SOR⁹, NR¹¹SO₂R⁹, NR¹¹SO₂NR⁹R¹⁰, optionally substituted C₁-C₈ alkyl, optionally substituted C₂-C₈ alkenyl, optionally substituted C₂-C₈ alkynyl, optionally substituted C₁-C₈ alkoxy, optionally substituted C₁-C₈alkoxyC₁-C₈alkyl, optionally substituted C₁-C₈alkylamino, optionally substituted C₁-C₈alkylaminoC₁-C₈alkyl, optionally substituted 3-10 membered carbocyclyl, optionally substituted 3-8 membered cycloalkoxy, optionally substituted 3-10 membered carbocyclylamino, optionally substituted 4-10 membered heterocyclyl, optionally substituted aryl, and optionally substituted heteroaryl, wherein

R⁹, R¹⁰, and R¹¹ are independently selected from hydrogen, optionally substituted C₁-C₈ alkyl, optionally substituted C₂-C₈ alkenyl, optionally substituted C₂-C₈ alkynyl, optionally substituted C₁-C₈alkoxyC₁-C₈alkyl, optionally substituted C₁-C₈alkylaminoC₁-C₈alkyl, optionally substituted 3-10 membered carbocyclyl, optionally substituted 4-10 membered heterocyclyl, optionally substituted aryl, and optionally substituted heteroaryl, or

R⁹ and R¹⁰, R⁹ and R¹¹ together with the atom to which they are connected form a 3-20 membered cycloalkyl or 4-20 membered heterocyclyl ring;

R² is connected to the “linker” moiety of the bivalent compound, and is selected from null, R″O, R″S, R″NR¹², R″OC(O), R″OC(O)O, R″OCONR¹², R″C(O), R″C(O)O, R″CONR¹², R″S(O), R″S(O)₂, R″SO₂NR¹², R″NR¹³C(O)O, R″NR¹³C(O), R″NR¹³C(O)NR¹², R″NR¹³S(O), R″NR¹³S(O)₂, R″NR¹³S(O)₂NR¹², optionally substituted C₁-C₈ alkylene, optionally substituted C₂-C₈ alkenylene, optionally substituted C₂-C₈ alkynylene, optionally substituted 3-10 membered carbocyclyl, optionally substituted 4-10 membered heterocyclyl, optionally substituted C₃-C₁₃ fused cycloalkyl, optionally substituted C₃-C₁₃ fused heterocyclyl, optionally substituted C₃-C₁₃ bridged cycloalkyl, optionally substituted C₃-C₁₃ bridged heterocyclyl, optionally substituted C₃-C₁₃ spiro cycloalkyl, optionally substituted C₃-C₁₃ spiro heterocyclyl, optionally substituted aryl, and optionally substituted heteroaryl, wherein

R″ is null, or a bivalent moiety selected from optionally substituted C₁-C₈ alkylene, optionally substituted C₂-C₈ alkenylene, optionally substituted C₂-C₈ alkynylene, optionally substituted C₁-C₈alkoxyC₁-C₈alkylene, optionally substituted C₁-C₈ haloalkylene, optionally substituted C₁-C₈ hydroxyalkylene, optionally substituted 3-10 membered carbocyclyl, optionally substituted 4-10 membered heterocyclyl, optionally substituted C₃-C₁₃ fused cycloalkyl, optionally substituted C₃-C₁₃ fused heterocyclyl, optionally substituted C₃-C₁₃ bridged cycloalkyl, optionally substituted C₃-C₁₃ bridged heterocyclyl, optionally substituted C₃-C₁₃ spiro cycloalkyl, optionally substituted C₃-C₁₃ spiro heterocyclyl, optionally substituted aryl, and optionally substituted heteroaryl;

R¹² and R¹³ are independently selected from optionally substituted C₁-C₈ alkyl, optionally substituted C₂-C₈ alkenyl, optionally substituted C₂-C₈ alkynyl, optionally substituted C₁-C₈alkoxyC₁-C₈alkyl, optionally substituted C₁-C₈alkylaminoC₁-C₈alkyl, optionally substituted 3-10 membered carbocyclyl, optionally substituted 4-10 membered heterocyclyl, optionally substituted aryl, and optionally substituted heteroaryl, or

R¹² and R¹³ together with the atom to which they are connected form a 3-20 membered cycloalkyl or 4-20 membered heterocyclyl ring; and

R³ is selected from hydrogen, COR¹⁴, CO₂R¹⁴, CONR¹⁴R¹⁵, SOR¹⁴, SO₂R¹⁴, SO₂NR¹⁴R¹⁵, optionally substituted C₁-C₆ alkyl, optionally substituted C₂-C₆ alkenyl, optionally substituted C₂-C₆ alkynyl, optionally substituted 3-6 membered cycloalkyl, optionally substituted 4-6 membered heterocyclyl, optionally substituted aryl, and optionally substituted heteroaryl, wherein

R¹⁴ and R¹⁵ are independently selected from hydrogen, optionally substituted C₁-C₆ alkyl, optionally substituted C₂-C₆ alkenyl, optionally substituted C₂-C₆ alkynyl, optionally substituted 3-6 membered cycloalkyl, optionally substituted 4-6 membered heterocyclyl, optionally substituted aryl, and optionally substituted heteroaryl, or

R¹⁴ and R¹⁵ together with the atom to which they are connected form a 4-20 membered heterocyclyl ring.

In another embodiment, X¹ is C; and X² and X³ are N. The FORMULA I is FORMULA 1A:

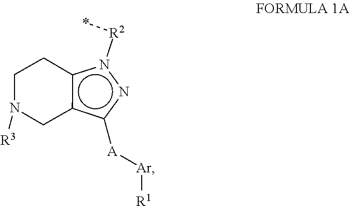

wherein

A, Ar, R¹, R² and R³ are the same as in FORMULA 1.

In another embodiment, A-Ar—R¹ is a moiety of formulae A1:

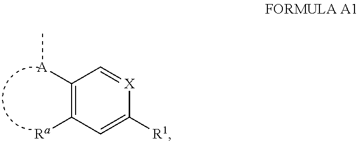

wherein

A and R¹ are the same as in FORMULA 1.

X is selected from CR′″ and N, wherein

R″′ and is selected from hydrogen, halogen, hydroxyl, amino, cyano, nitro, optionally substituted C₁-C₆ alkyl, optionally substituted C₂-C₆ alkenyl, optionally substituted C₂-C₆ alkynyl, optionally substituted C₁-C₆ alkoxy, optionally substituted C₁-C₆ alkylamino, optionally substituted 3-6 membered cycloalkyl, optionally substituted 3-6 membered cycloalkoxy, optionally substituted 3-6 membered cycloalkylamino, optionally substituted 4-6 membered heterocyclyl, optionally substituted aryl, and optionally substituted heteroaryl; and

R^{a} is optionally formed a ring with A, and is selected from null, hydrogen, halogen, R^{b}NR¹⁶, R^{b}OR¹⁶, R^{b}SR¹⁶, R^{b}NR¹⁶R¹⁷, R^{b}OCOR¹⁶, R^{b}OCO₂R¹⁶, R^{b}OCONR¹⁶R¹⁷, R^{b}COR¹⁶, R^{b}CO₂R¹⁶, R^{b}CONR¹⁶R¹⁷, R^{b}SOR¹⁶, R^{b}SO₂R¹⁶, R^{b}SO₂NR¹⁶R¹⁷, R^{b}NR¹⁸CO₂R¹⁶, R^{b}NR¹⁸COR¹⁶, R^{b}NR¹⁸C(O)NR¹⁶R¹⁷, R^{b}NR¹⁸SOR¹⁶, R^{b}NR¹⁸SO₂R¹⁶, R^{b}NR¹⁸SO₂NR¹⁶R¹⁷, optionally substituted C₁-C₈ alkyl, optionally substituted C₁-C₈ alkylene, optionally substituted C₂-C₈ alkenyl, optionally substituted C₂-C₈ alkenylene, optionally substituted C₂-C₈ alkynyl, optionally substituted C₂-C₈ alkynylene, optionally substituted 3-10 membered carbocyclyl, optionally substituted 4-10 membered heterocyclyl, optionally substituted aryl, and optionally substituted heteroaryl, wherein

R^{b} is null, or a bivalent or trivalent moiety selected from optionally substituted C₁-C₈ alkylene, optionally substituted C₂-C₈ alkenylene, optionally substituted C₂-C₈ alkynylene, optionally substituted C₁-C₈alkoxyC₁-C₈alkylene, optionally substituted C₁-C₈alkylaminoC₁-C₈alkylene, optionally substituted C₁-C₈ haloalkylene, optionally substituted C₁-C₈ hydroxyalkylene, optionally substituted 3-10 membered carbocyclyl, optionally substituted 4-10 membered heterocyclyl, optionally substituted aryl, and optionally substituted heteroaryl;

R¹⁶, R¹⁷, and R¹⁸ are independently selected from null, hydrogen, optionally substituted C₁-C₈ alkyl, optionally substituted C₂-C₈ alkenyl, optionally substituted C₂-C₈ alkynyl, optionally substituted C₁-C₈alkoxyC₁-C₈alkyl, optionally substituted C₁-C₈alkylaminoC₁-C₈alkyl, optionally substituted 3-10 membered carbocyclyl, optionally substituted 4-10 membered heterocyclyl, optionally substituted aryl, and optionally substituted heteroaryl, or

R¹⁶ and R¹⁷, R¹⁶ and R¹⁸ together with the atom to which they are connected form a 3-20 membered cycloalkyl or heterocyclyl ring.

In another embodiment, A is null.

In another embodiment, A is null; Ar is a bicyclic aryl or a bicyclic heteroaryl; and A-Ar—R¹ is a moiety of FORMULAE A2 or A3:

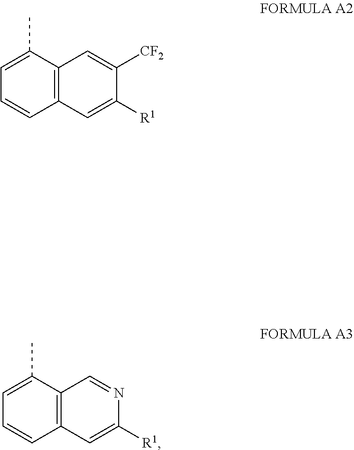

wherein

R¹ is the same as in FORMULA 1.

In another embodiment, A is NR⁴, wherein

R⁴ is selected from hydrogen, optionally substituted C₁-C₈ alkyl, optionally substituted C₂-C₈ alkenyl, optionally substituted C₂-C₈ alkynyl, optionally substituted C₁-C₈alkoxyC₁-C₈alkyl, optionally substituted C₁-C₈alkylaminoC₁-C₈alkyl, optionally substituted 3-10 membered carbocyclyl, optionally substituted 4-10 membered heterocyclyl, optionally substituted aryl, and optionally substituted heteroaryl.

In another embodiment, A is NR⁴; and A-Ar—R¹ is a moiety of FORMULAE A4, A5 or A6:

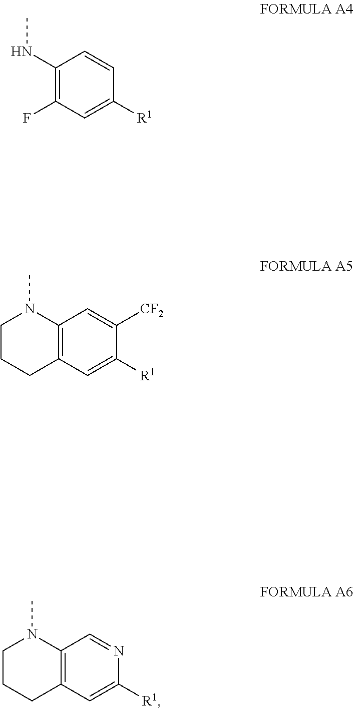

wherein

R¹ is the same as in FORMULA 1.

In another embodiment, R¹ is selected from optionally substituted 3-10 membered carbocyclyl, optionally substituted 4-10 membered heterocyclyl, optionally substituted aryl and optionally substituted heteroaryl.

In another embodiment, R¹ is selected from optionally substituted aryl and optionally substituted heteroaryl.

In another embodiment, R¹ is selected from optionally substituted pyrazole and optionally substituted pyridinyl.

In another embodiment, R² is selected from optionally substituted C₁-C₈ alkylene, optionally substituted 3-10 membered carbocyclyl, optionally substituted 4-8 membered heterocycly, optionally substituted aryl, and optionally substituted heteroaryl.

In another embodiment, R³ is selected from COR¹⁴ and CONR¹⁴R¹⁵.

In another embodiment, R³ is selected from COMe and CONHMe.

In another embodiment, the CBP/P300 ligand comprises a moiety of FORMULA 2:

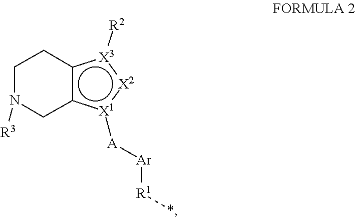

wherein

the linker moiety of the bivalent compound is attached to R¹; and

X¹ and X³ are independently selected from C and N, with the proviso that at least one of X¹ and X³ is C and at most only one of X¹ and X³ is N;

X² is selected from CR′, O, and NR′, wherein

R′ is selected from H, optionally substituted C₁-C₈ alkyl, and optionally substituted 3-10 membered carbocyclyl;

A is selected from null, CR⁴R⁵, CO, O, S, SO, SO₂, and NR⁴, wherein

R⁴ and R⁵ are independently selected from hydrogen, halogen, hydroxyl, amino, cyano, nitro, optionally substituted C₁-C₈ alkyl, optionally substituted C₂-C₈ alkenyl, optionally substituted C₂-C₈ alkynyl, optionally substituted C₁-C₈ alkoxy, optionally substituted C₁-C₈alkoxyC₁-C₈alkyl, optionally substituted C₁-C₈ alkylamino, optionally substituted C₁-C₈alkylaminoC₁-C₈alkyl, optionally substituted 3-10 membered carbocyclyl, optionally substituted 3-8 membered cycloalkoxy, optionally substituted 3-10 membered carbocyclylamino, optionally substituted 4-10 membered heterocyclyl, optionally substituted aryl, and optionally substituted heteroaryl;

Ar and R⁴, Ar and R⁵, and/or R⁴ and R⁵ together with the atom to which they are connected form an optionally substituted 3-20 membered cycloalkyl or 4-20 membered heterocyclyl ring;

Ar is selected from aryl, heteroaryl, bicyclic aryl, bicyclic heteroaryl, tricyclic aryl, tricyclic heteroaryl groups, each of which is substituted with R¹ and optionally substituted with one or more substituents independently selected from hydrogen, halogen, oxo, CN, NO₂, OR⁶, SR⁶, NR⁶R⁷, OCOR⁶, OCO₂R⁶, OCONR⁶R⁷, COR⁶, CO₂R⁶, CONR⁶R⁷, SOR⁶, SO₂R⁶, SO₂NR⁶R⁷, NR⁸CO₂R⁶, NR⁸COR⁶, NR⁸C(O)NR⁶R⁷, NR⁸SOR⁶, NR⁸SO₂R⁶, NR⁸SO₂NR⁶R⁷, optionally substituted C₁-C₈ alkyl, optionally substituted C₂-C₈ alkenyl, optionally substituted C₂-C₈ alkynyl, optionally substituted C₁-C₈ alkoxy, optionally substituted C₁-C₈alkoxyC₁-C₈alkyl, optionally substituted C₁-C₈alkylamino, optionally substituted C₁-C₈alkylaminoC₁-C₈alkyl, optionally substituted 3-10 membered carbocyclyl, optionally substituted 3-8 membered cycloalkoxy, optionally substituted 3-10 membered carbocyclylamino, optionally substituted 4-10 membered heterocyclyl, optionally substituted aryl, and optionally substituted heteroaryl, wherein

R⁶, R⁷, and R⁸ are independently selected from hydrogen, optionally substituted C₁-C₈ alkyl, optionally substituted C₂-C₈ alkenyl, optionally substituted C₂-C₈ alkynyl, optionally substituted C₁-C₈alkoxyC₁-C₈alkyl, optionally substituted C₁-C₈alkylaminoC₁-C₈alkyl, optionally substituted 3-10 membered carbocyclyl, optionally substituted 4-10 membered heterocyclyl, optionally substituted aryl, and optionally substituted heteroaryl, or

R⁶ and R⁷, R⁶ and R⁸ together with the atom to which they are connected form a 4-20 membered heterocyclyl ring; and

R¹ is connected to the “linker” moiety of the bivalent compound, and R¹ is selected from null, R″O, R″S, R″NR⁹, R″OC(O), R″OC(O)O, R″OCONR⁹, R″C(O), R″C(O)O, R″CONR⁹, R″S(O), R″S(O)₂, R″SO₂NR⁹, R″NR¹⁰C(O)O, R″NR¹⁰C(O), R″NR¹⁰C(O)NR⁹, R″NR¹⁰S(O), R″NR¹⁰S(O)₂, R″NR¹⁰S(O)₂NR⁹, optionally substituted C₁-C₈ alkylene, optionally substituted C₂-C₈ alkenylene, optionally substituted C₂-C₈ alkynylene, optionally substituted 3-10 membered carbocyclyl, optionally substituted 4-8 membered heterocycly, optionally substituted aryl, and optionally substituted heteroaryl, wherein

R″ is null, or a bivalent moiety selected from optionally substituted C₁-C₈ alkylene, optionally substituted C₂-C₈ alkenylene, optionally substituted C₂-C₈ alkynylene, optionally substituted C₁-C₈alkoxyC₁-C₈alkylene, optionally substituted C₁-C₈ haloalkylene, optionally substituted C₁-C₈ hydroxyalkylene, optionally substituted 3-10 membered carbocyclyl, optionally substituted 4-10 membered heterocyclyl, optionally substituted C₃-C₁₃ fused cycloalkyl, optionally substituted C₃-C₁₃ fused heterocyclyl, optionally substituted C₃-C₁₃ bridged cycloalkyl, optionally substituted C₃-C₁₃ bridged heterocyclyl, optionally substituted C₃-C₁₃ spiro cycloalkyl, optionally substituted C₃-C₁₃ spiro heterocyclyl, optionally substituted aryl, and optionally substituted heteroaryl;

R⁹ and R¹⁰ are independently selected from optionally substituted C₁-C₈ alkyl, optionally substituted C₂-C₈ alkenyl, optionally substituted C₂-C₈ alkynyl, optionally substituted C₁-C₈alkoxyC₁-C₈alkyl, optionally substituted C₁-C₈alkylaminoC₁-C₈alkyl, optionally substituted 3-10 membered carbocyclyl, optionally substituted 4-10 membered heterocyclyl, optionally substituted aryl, and optionally substituted heteroaryl, or

R⁹ and R¹⁰ together with the atom to which they are connected form a 3-20 membered cycloalkyl or 4-20 membered heterocyclyl ring; and

R² is selected from hydrogen, halogen, CN, NO₂, OR¹¹, SR¹¹, NR¹¹R¹², OCOR¹¹, OCO₂R¹¹, OCONR¹¹R¹², COR¹¹, CO₂R¹¹, CONR¹¹R¹², SOR¹¹, SO₂R¹¹, SO₂NR¹¹R¹², NR¹³CO₂R¹¹, NR¹³COR¹¹, NR¹³C(O)NR¹¹R¹², NR¹³SOR¹¹, NR¹³SO₂R¹¹, NR¹³SO₂NR¹¹R¹², optionally substituted C₁-C₈ alkyl, optionally substituted C₂-C₈ alkenyl, optionally substituted C₂-C₈ alkynyl, optionally substituted C₁-C₈ alkoxy, optionally substituted C₁-C₈alkoxyC₁-C₈alkyl, optionally substituted C₁-C₈alkylamino, optionally substituted C₁-C₈alkylaminoC₁-C₈alkyl, optionally substituted 3-10 membered carbocyclyl, optionally substituted 3-8 membered cycloalkoxy, optionally substituted 3-10 membered carbocyclylamino, optionally substituted 4-10 membered heterocyclyl, optionally substituted aryl, and optionally substituted heteroaryl, wherein

R¹¹, R¹², and R¹³ are independently selected from hydrogen, optionally substituted C₁-C₈ alkyl, optionally substituted C₂-C₈ alkenyl, optionally substituted C₂-C₈ alkynyl, optionally substituted C₁-C₈alkoxyC₁-C₈alkyl, optionally substituted C₁-C₈alkylaminoC₁-C₈alkyl, optionally substituted 3-10 membered carbocyclyl, optionally substituted 4-10 membered heterocyclyl, optionally substituted aryl, and optionally substituted heteroaryl, or

R¹¹ and R¹², R¹¹ and R¹³ together with the atom to which they are connected form a 3-20 membered cycloalkyl or 4-20 membered heterocyclyl ring; and

R³ is selected from hydrogen, COR¹⁴, CO₂R¹⁴, CONR¹⁴R¹⁵, SOR¹⁴, SO₂R¹⁴, SO₂NR¹⁴R¹⁵, optionally substituted C₁-C₆ alkyl, optionally substituted C₂-C₆ alkenyl, optionally substituted C₂-C₆ alkynyl, optionally substituted 3-6 membered cycloalkyl, optionally substituted 4-6 membered heterocyclyl, wherein

R¹⁴ and R¹⁵ are independently selected from hydrogen, optionally substituted C₁-C₆ alkyl, optionally substituted C₂-C₆ alkenyl, optionally substituted C₂-C₆ alkynyl, optionally substituted 3-6 membered cycloalkyl, and optionally substituted 4-6 membered heterocyclyl, or

R¹⁴ and R¹⁵ together with the atom to which they are connected form a 4-6 membered heterocyclyl ring.

In another embodiment, X¹ is C; and X² and X³ are N. The FORMULA 2 is FORMULA 2A:

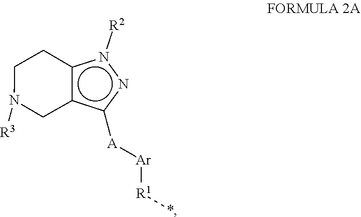

wherein

A, Ar, R¹, R² and R³ are the same as in FORMULA 2.

In another embodiment, A-Ar—R¹ is a moiety of formulae B1:

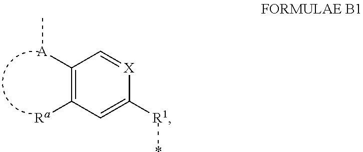

wherein

* indicates the connection to the linker moiety of the bivalent compound;

A and R¹ are the same as in FORMULA 2;

X is selected from CR′″ and N, wherein

R″′ and is selected from hydrogen, halogen, hydroxyl, amino, cyano, nitro, optionally substituted C₁-C₆ alkyl, optionally substituted C₂-C₆ alkenyl, optionally substituted C₂-C₆ alkynyl, optionally substituted C₁-C₆ alkoxy, optionally substituted C₁-C₆ alkylamino, optionally substituted 3-6 membered cycloalkyl, optionally substituted 3-6 membered cycloalkyl, optionally substituted 3-6 membered cycloalkoxy, optionally substituted 3-6 membered cycloalkylamino, optionally substituted 4-6 membered heterocyclyl; and

R^{a} optionally forms a ring with A, and is selected from null, hydrogen, halogen, R^{b}NR¹⁶, R^{b}OR¹⁶, R^{b}SR¹⁶, R^{b}NR¹⁶R¹⁷, R^{b}OCOR¹⁶, R^{b}OCO₂R¹⁶, R^{b}OCONR¹⁶R¹⁷, R^{b}COR¹⁶, R^{b}CO₂R¹⁶, R^{b}CONR¹⁶R¹⁷, R^{b}SOR¹⁶, R^{b}SO₂R¹⁶, R^{b}SO₂NR¹⁶R¹⁷, R^{b}NR¹⁸CO₂R¹⁶, R^{b}NR¹⁸COR¹⁶, R^{b}NR¹⁸C(O)NR¹⁶R¹⁷, R^{b}NR¹⁸SOR¹⁶, R^{b}NR¹⁸SO₂R¹⁶, R^{b}NR¹⁸SO₂NR¹⁶R¹⁷, optionally substituted C₁-C₈ alkyl, optionally substituted C₁-C₈ alkylene, optionally substituted C₂-C₈ alkenyl, optionally substituted C₂-C₈ alkenylene, optionally substituted C₂-C₈ alkynyl, optionally substituted C₂-C₈ alkynylene, optionally substituted 3-10 membered carbocyclyl, optionally substituted 4-10 membered heterocyclyl, optionally substituted aryl, and optionally substituted heteroaryl, wherein

R^{b} is null, or a bivalent or trivalent moiety selected from optionally substituted C₁-C₈ alkylene, optionally substituted C₂-C₈ alkenylene, optionally substituted C₂-C₈ alkynylene, optionally substituted C₁-C₈alkoxyC₁-C₈alkylene, optionally substituted C₁-C₈alkylaminoC₁-C₈alkylene, optionally substituted C₁-C₈ haloalkylene, optionally substituted C₁-C₈ hydroxyalkylene, optionally substituted 3-10 membered carbocyclyl, optionally substituted 4-10 membered heterocyclyl, optionally substituted aryl, and optionally substituted heteroaryl;

R¹⁶, R¹⁷, and R¹⁸ are independently selected from null, a bond, hydrogen, optionally substituted C₁-C₈ alkyl, optionally substituted C₂-C₈ alkenyl, optionally substituted C₂-C₈ alkynyl, optionally substituted C₁-C₈alkoxyC₁-C₈alkyl, optionally substituted C₁-C₈alkylaminoC₁-C₈alkyl, optionally substituted 3-10 membered carbocyclyl, optionally substituted 4-10 membered heterocyclyl, optionally substituted aryl, and optionally substituted heteroaryl, or

R¹⁶ and R¹⁷, R¹⁶ and R¹⁸ together with the atom to which they are connected form a 3-20 membered cycloalkyl or 4-20 membered heterocyclyl ring.

In another embodiment, A is null.

In another embodiment, A is null; Ar is a bicyclic aryl or a bicyclic heteroaryl; and A-Ar—R¹ is a moiety of FORMULAE B2 or B3:

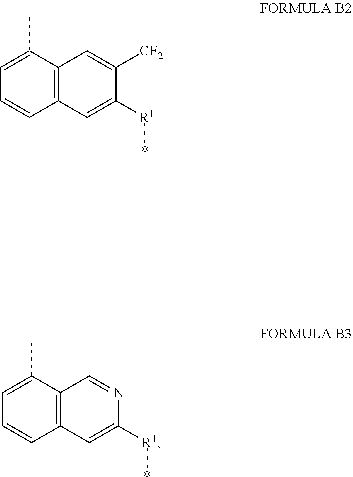

wherein

* indicates the connection to the linker moiety of the bivalent compound; and

R¹ is the same as in FORMULA 2.

In another embodiment, A is NR⁴, wherein

R⁴ is selected from hydrogen, optionally substituted C₁-C₈ alkyl, optionally substituted C₂-C₈ alkenyl, optionally substituted C₂-C₈ alkynyl, optionally substituted C₁-C₈alkoxyC₁-C₈alkyl, optionally substituted C₁-C₈alkylaminoC₁-C₈alkyl, optionally substituted 3-10 membered carbocyclyl, optionally substituted 4-10 membered heterocyclyl, optionally substituted aryl, and optionally substituted heteroaryl.

In another embodiment, A is NR⁴; and A-Ar—R¹ is a moiety of FORMULAE B4, B5 or B6:

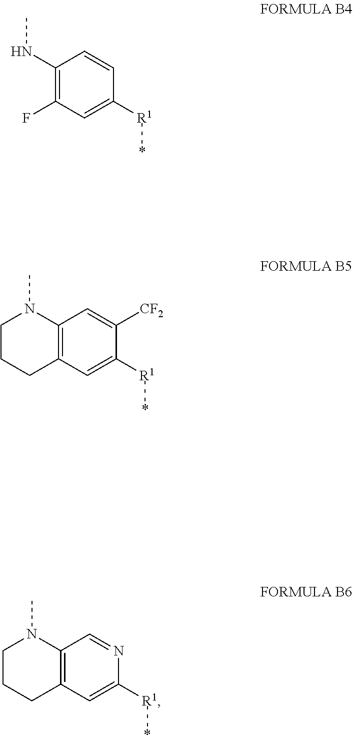

wherein

* indicates the connection to the linker moiety of the bivalent compound; and

R¹ is the same as in FORMULA 2.

In another embodiment, R¹ is selected from optionally substituted 3-10 membered carbocyclylene, optionally substituted 4-10 membered heterocyclyl, optionally substituted aryl, and optionally substituted heteroaryl.

In another embodiment, R¹ is selected from optionally substituted aryl and optionally substituted heteroaryl.

In another embodiment, R¹ is selected from optionally substituted pyrazole and optionally substituted pyridinyl.

In another embodiment, R² is selected from optionally substituted C₁-C₈ alkyl, optionally substituted 3-10 membered carbocyclyl, optionally substituted 4-10 membered heterocyclyl, optionally substituted aryl, and optionally substituted heteroaryl.

In another embodiment, R³ is selected from COR¹⁴ and CONR¹⁴R¹⁵.

In another embodiment, R³ is selected from COMe and CONHMe.

In another embodiment, the CBP/P300 ligand is derived from any of the following:

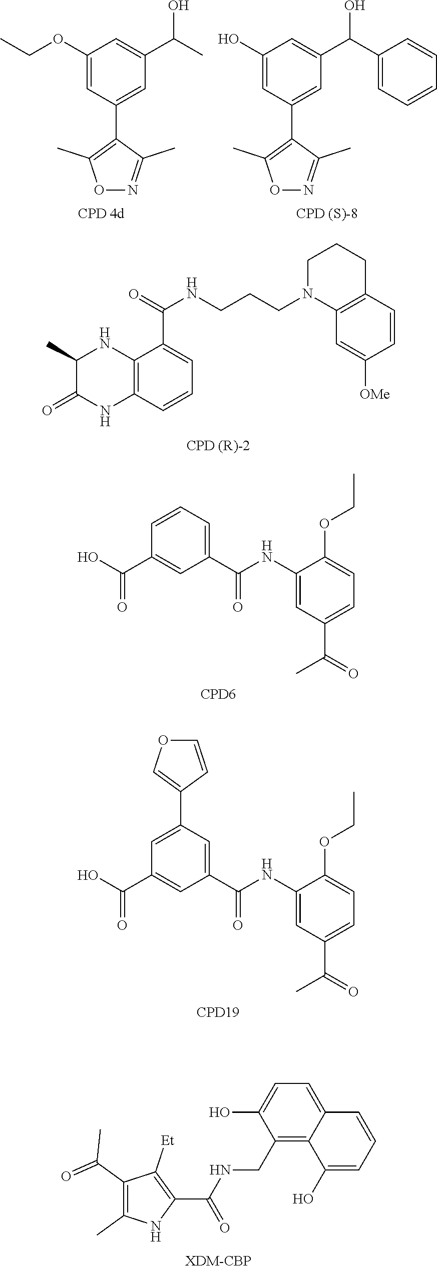

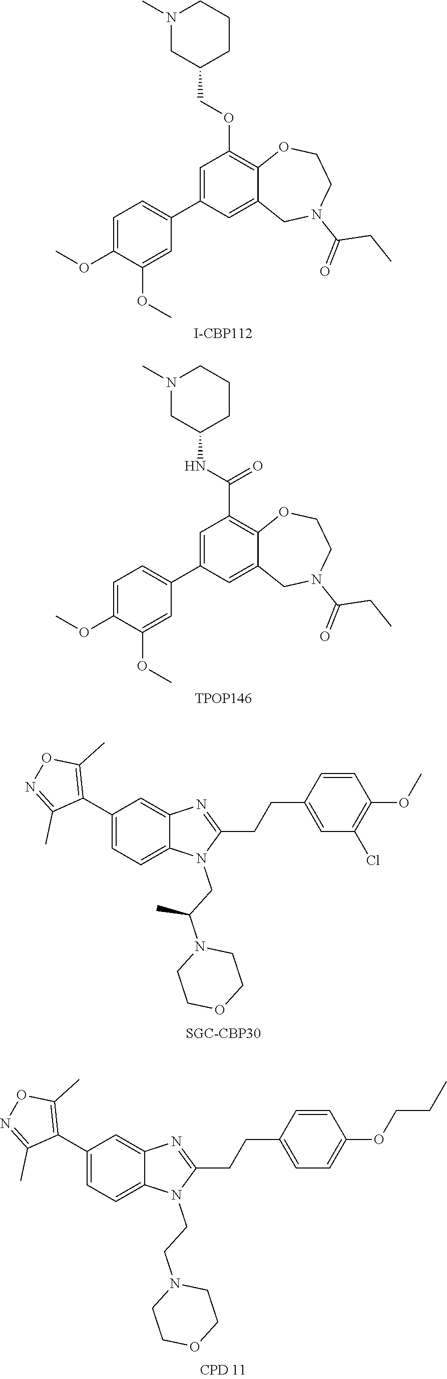

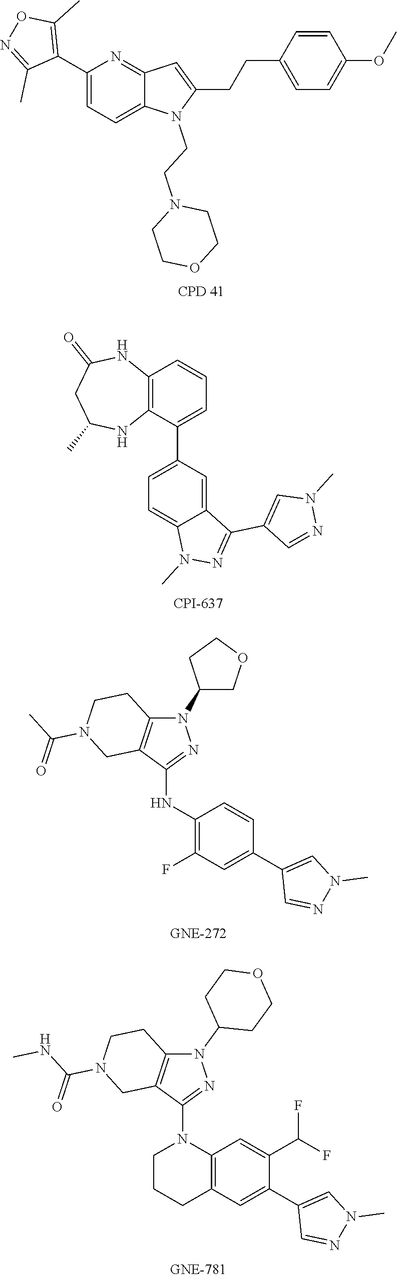

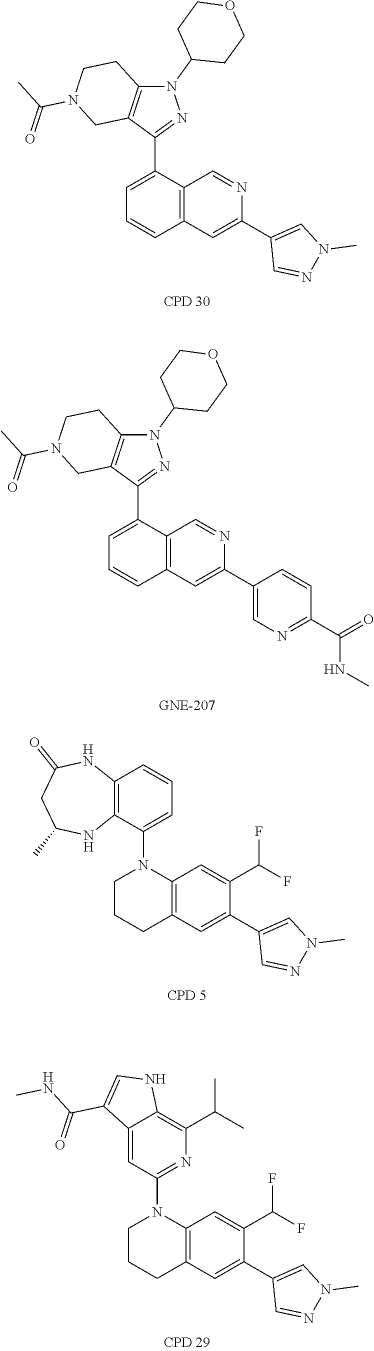

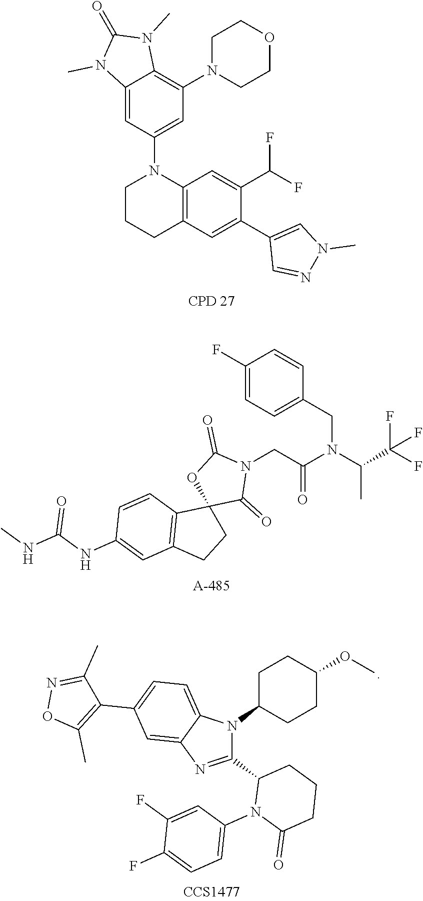

In another embodiment, the CBP/P300 ligand is derived from the following CBP/P300 inhibitors: C646, naphthol-AS-E, compound 1-10, MYBMIM, CCS1477, ICG-001, YH249, YH250, HBS1, OHM1, and KCN1.

In another embodiment, the CBP/P300 ligand is selected from the group consisting of:

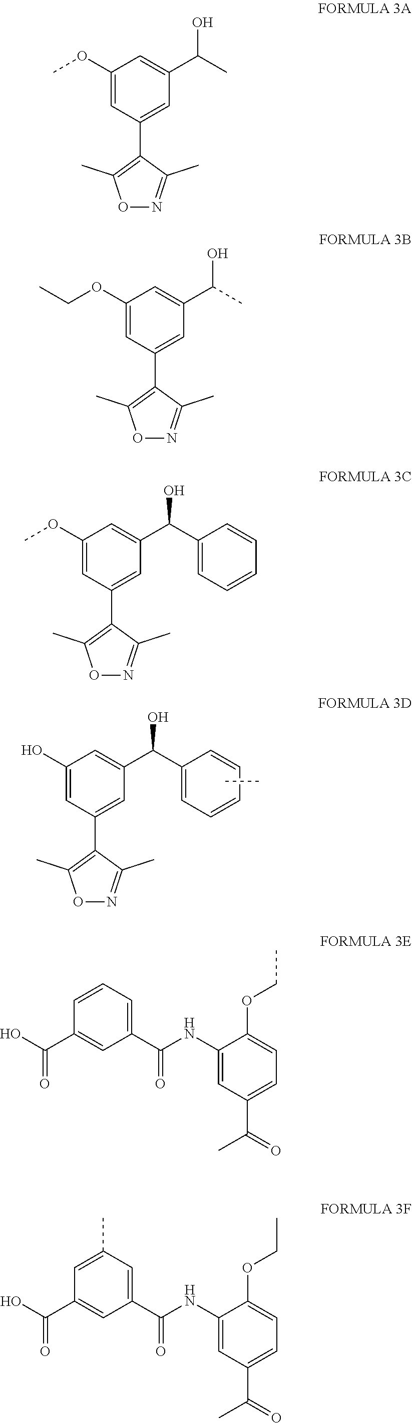

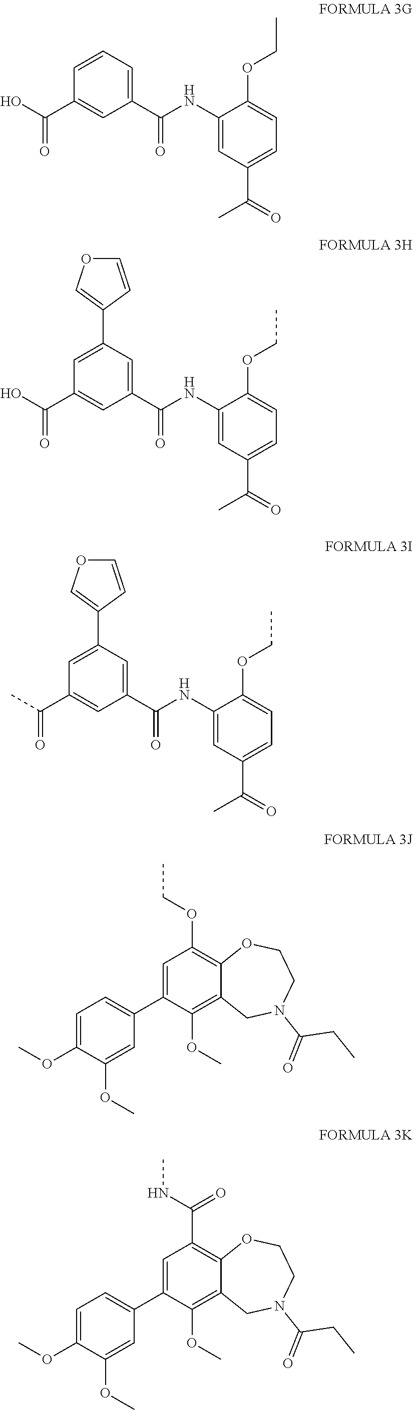

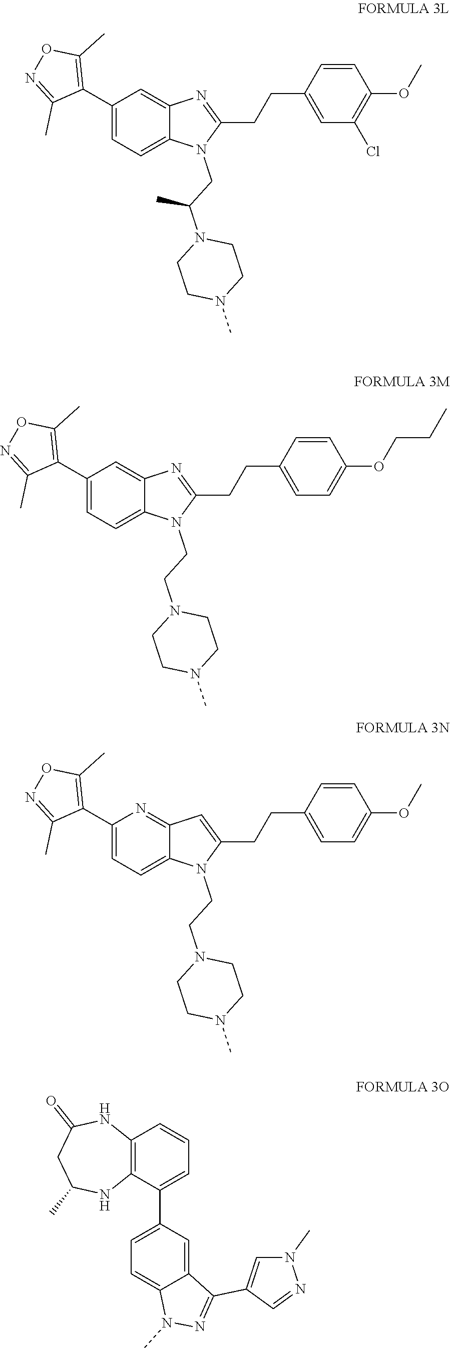

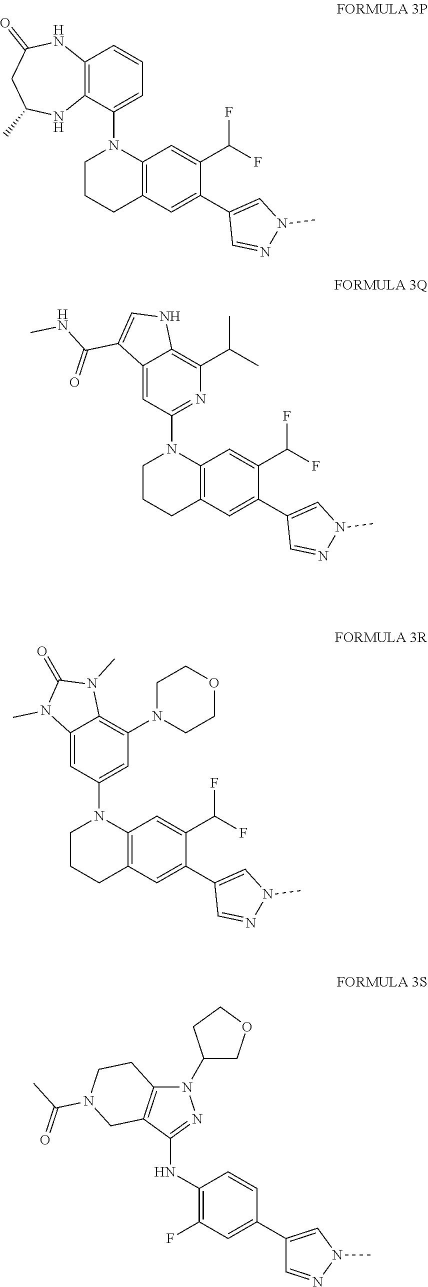

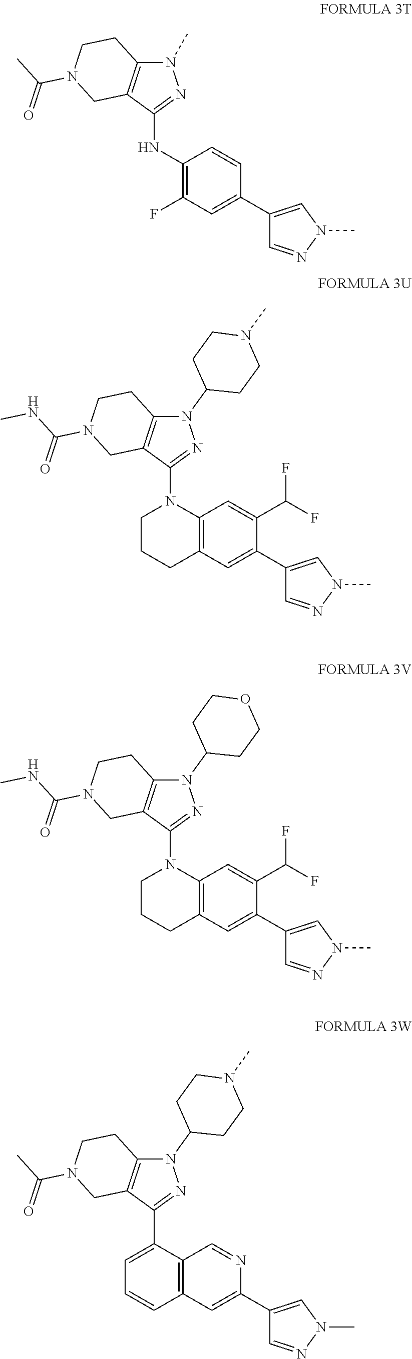

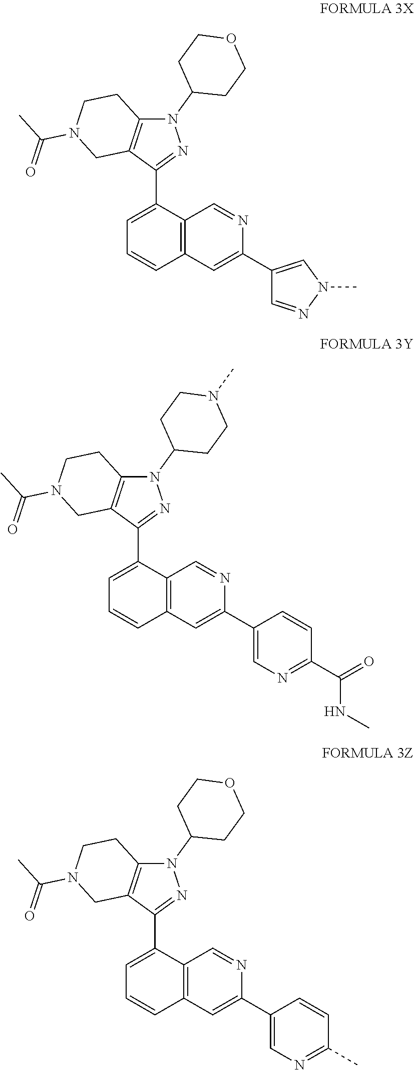

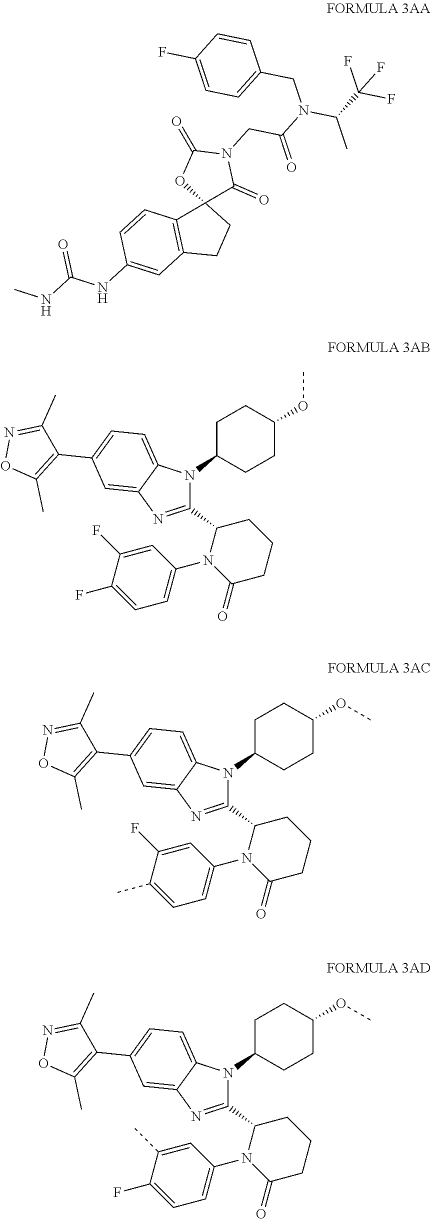

In another embodiment, the degradation tag is a moiety selected from the group consisting of FORMULAE 5A, 5B, 5C, and 5D:

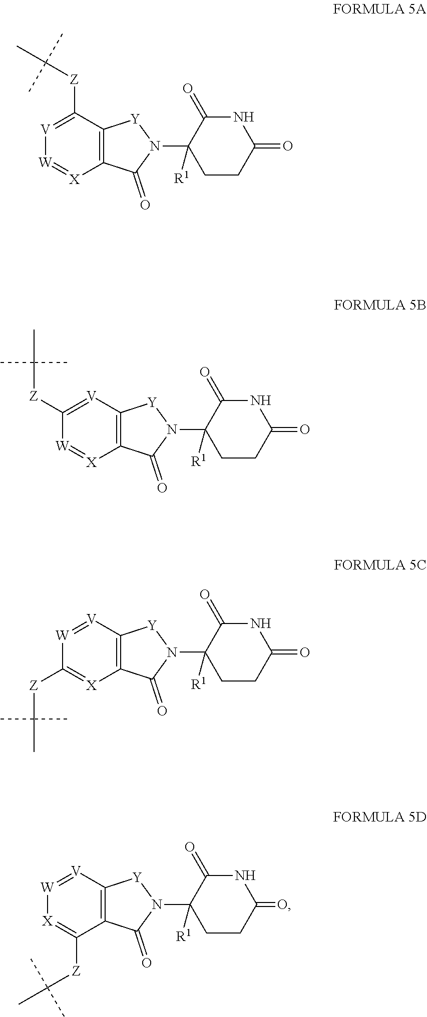

wherein

V, W, and X are independently selected from CR² and N;

Y is selected from CO, CR³R⁴, N═CR³, and N═N;

Z is selected from null, CO, CR⁵R⁶, NR⁵, O, optionally substituted C₁-C₁₀ alkylene, optionally substituted C₁-C₁₀ alkenylene, optionally substituted C₁-C₁₀ alkynylene, optionally substituted 3-10 membered carbocyclyl, optionally substituted 4-10 membered heterocyclyl, optionally substituted C₃-C₁₃ fused cycloalkyl, optionally substituted C₃-C₁₃ fused heterocyclyl, optionally substituted C₃-C₁₃ bridged cycloalkyl, optionally substituted C₃-C₁₃ bridged heterocyclyl, optionally substituted C₃-C₁₃ spiro cycloalkyl, optionally substituted C₃-C₁₃ spiro heterocyclyl, optionally substituted aryl, and optionally substituted heteroaryl; preferably, Z is selected from null, CH₂, CH═CH, C≡C, NH and O;

R¹, and R² are independently selected from hydrogen, halogen, cyano, nitro, optionally substituted C₁-C₆ alkyl, optionally substituted 3 to 6 membered carbocyclyl, and optionally substituted 4 to 6 membered heterocyclyl;

R³, and R⁴ are independently selected from hydrogen, halogen, cyano, nitro, optionally substituted C₁-C₆ alkyl, optionally substituted 3 to 6 membered carbocyclyl, and optionally substituted 4 to 6 membered heterocyclyl; or R³ and R⁴ together with the atom to which they are connected form a 3-6 membered carbocyclyl, or 4-6 membered heterocyclyl; and

R⁵ and R⁶ are independently selected from null, hydrogen, halogen, oxo, hydroxyl, amino, cyano, nitro, optionally substituted C₁-C₆ alkyl, optionally substituted 3 to 6 membered carbocyclyl, and optionally substituted 4 to 6 membered heterocyclyl; or R⁵ and R⁶ together with the atom to which they are connected form a 3-6 membered carbocyclyl, or 4-6 membered heterocyclyl.

In another embodiment, the degradation tag is a moiety selected from the group consisting of FORMULAE 5A, 5B, 5C, and 5D:

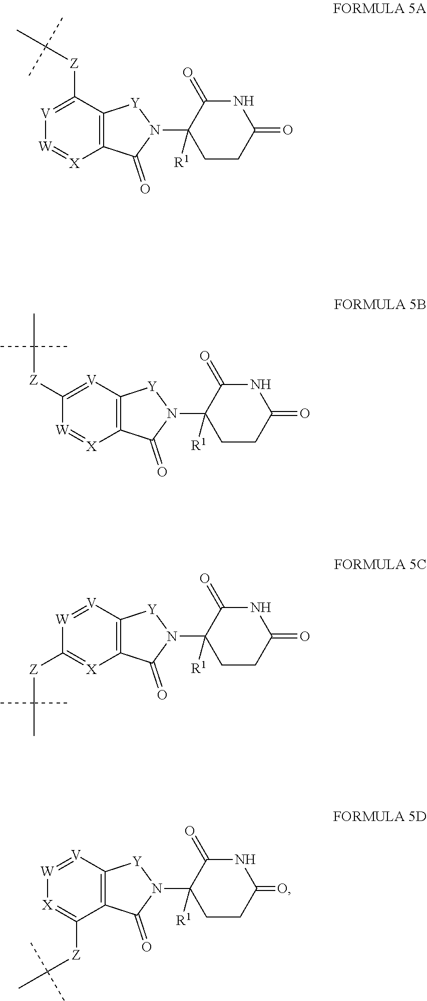

wherein

V, W, and X are independently selected from CR² and N;

Y is selected from CO, CH₂, and N═N;

Z is selected from CH₂, NH and O; and

R¹ and R² are independently selected from hydrogen, halogen, cyano, nitro, and C₁-C₅ alkyl. In another embodiment, the degradation tag is a moiety selected from the group consisting of FORMULAE 5E, 5F, 5G, 5H, and 5I:

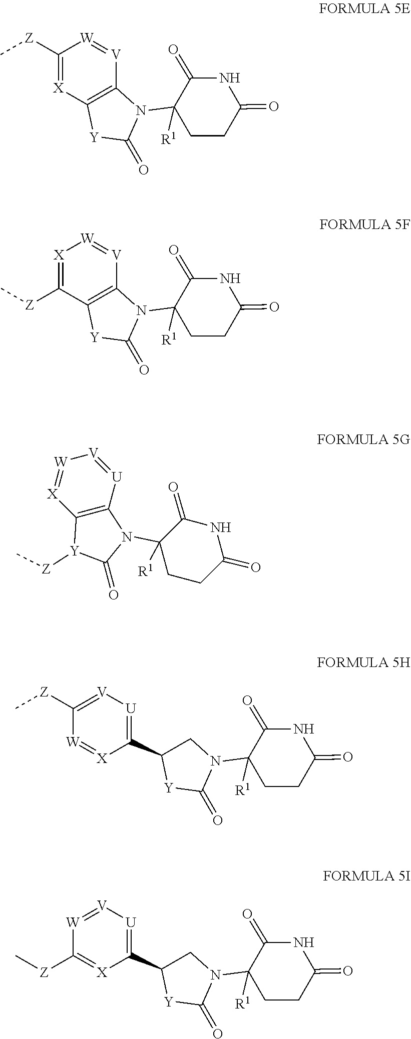

wherein

U, V, W, and X are independently selected from CR² and N;

Y is selected from CR³R⁴, NR³ and 0; preferably, Y is selected from CH₂, NH, NCH₃ and O;

Z is selected from null, CO, CR⁵R⁶, NR⁵, O, optionally substituted C₁-C₁₀ alkylene, optionally substituted C₁-C₁₀ alkenylene, optionally substituted C₁-C₁₀ alkynylene, optionally substituted 3-10 membered carbocyclyl, optionally substituted 4-10 membered heterocyclyl, optionally substituted C₃-C₁₃ fused cycloalkyl, optionally substituted C₃-C₁₃ fused heterocyclyl, optionally substituted C₃-C₁₃ bridged cycloalkyl, optionally substituted C₃-C₁₃ bridged heterocyclyl, optionally substituted C₃-C₁₃ spiro cycloalkyl, optionally substituted C₃-C₁₃ spiro heterocyclyl, optionally substituted aryl, and optionally substituted heteroaryl; preferably, Z is selected from null, CH₂, CH═CH, C≡C, NH and O;

R¹, and R² are independently selected from hydrogen, halogen, cyano, nitro, optionally substituted C₁-C₆ alkyl, optionally substituted 3 to 6 membered carbocyclyl, and optionally substituted 4 to 6 membered heterocyclyl;

R³, and R⁴ are independently selected from hydrogen, halogen, cyano, nitro, optionally substituted C₁-C₆ alkyl, optionally substituted 3 to 6 membered carbocyclyl, and optionally substituted 4 to 6 membered heterocyclyl; or R³ and R⁴ together with the atom to which they are connected form a 3-6 membered carbocyclyl, or 4-6 membered heterocyclyl; and

R⁵ and R⁶ are independently selected from null, hydrogen, halogen, oxo, hydroxyl, amino, cyano, nitro, optionally substituted C₁-C₆ alkyl, optionally substituted 3 to 6 membered carbocyclyl, and optionally substituted 4 to 6 membered heterocyclyl; or R⁵ and R⁶ together with the atom to which they are connected form a 3-6 membered carbocyclyl, or 4-6 membered heterocyclyl.

In one embodiment, the degradation tag is a moiety selected from the group consisting of FORMULAE 5J, 5K, 5L, 5M, 5N, 5O, 5P, and 5Q:

wherein

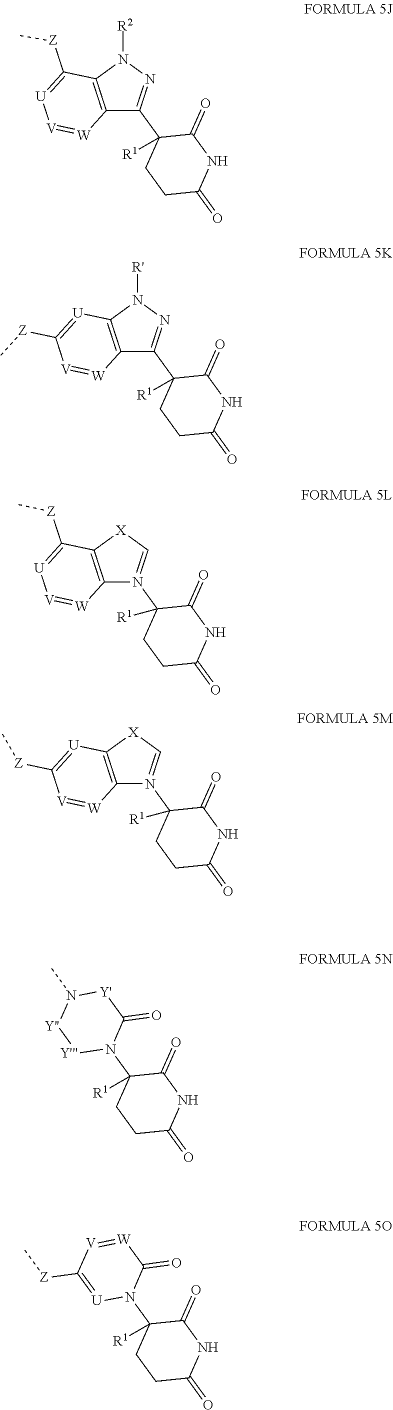

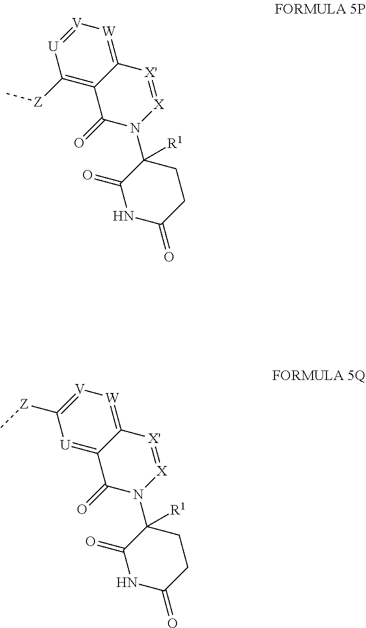

U, V, W, Y, X, Z, R¹, and R² are defined as in FORMULAE 5E, 5F, 5G, 5H, or 5I;

Y′, Y″, and Y′″ are independently selected from CR³R⁴;

X′ are independently selected from CR² and N;

R′ is selected from hydrogen, optionally substituted C₁-C₆ alkyl, optionally substituted 3 to 6 membered carbocyclyl, and optionally substituted 4 to 6 membered heterocyclyl.

In one embodiment, the degradation tag is a moiety of FORMULA 6A:

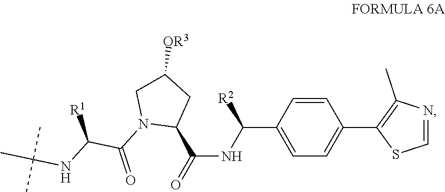

wherein

R¹ and R² are independently selected from hydrogen, hydroxyl, amino, cyano, nitro, optionally substituted C₁-C₈ alkyl, optionally substituted C₂-C₈ alkenyl, and optionally substituted C₂-C₈ alkynyl, optionally substituted C₁-C₈alkoxyC₁-C₈alkyl, optionally substituted C₁-C₈ haloalkyl, optionally substituted C₁-C₈ hydroxyalkyl, optionally substituted C₁-C₈ aminoalkyl, optionally substituted C₁-C₈alkylaminoC₁-C₈alkyl, optionally substituted 3-10 membered carbocyclyl, and optionally substituted 4-10 membered heterocyclyl.

R³ is selected from hydrogen, optionally substituted C(O)C₁-C₈ alkyl, optionally substituted C(O)C₁-C₈alkoxyC₁-C₈alkyl, optionally substituted C(O)C₁-C₈ haloalkyl, optionally substituted C(O)C₁-C₈ hydroxyalkyl, optionally substituted C(O)C₁-C₈ aminoalkyl, optionally substituted C(O)C₁-C₈alkylaminoC₁-C₈alkyl, optionally substituted C(O)(3-10 membered carbocyclyl), optionally substituted C(O)(4-10 membered heterocyclyl), optionally substituted C(O)C₂-C₈ alkenyl, optionally substituted C(O)C₂-C₈ alkynyl, optionally substituted C(O)OC₁-C₈alkoxyC₁-C₈alkyl, optionally substituted C(O)OC₁-C₈ haloalkyl, optionally substituted C(O)OC₁-C₈ hydroxyalkyl, optionally substituted C(O)OC₁-C₈ aminoalkyl, optionally substituted C(O)OC₁-C₈alkylaminoC₁-C₈alkyl, optionally substituted C(O)O(3-10 membered carbocyclyl), optionally substituted C(O)O(4-10 membered heterocyclyl), optionally substituted C(O)OC₂-C₈ alkenyl, optionally substituted C(O)OC₂-C₈ alkynyl, optionally substituted C(O)NC i-C₈alkoxyC₁-C₈alkyl, optionally substituted C(O)NC₁-C₈ haloalkyl, optionally substituted C(O)NC₁-C₈ hydroxyalkyl, optionally substituted C(O)NC₁-C₈ aminoalkyl, optionally substituted C(O)NC₁-C₈alkylaminoC₁-C₈alkyl, optionally substituted C(O)N(3-10 membered carbocyclyl), optionally substituted C(O)N(4-10 membered heterocyclyl), optionally substituted C(O)NC₂-C₈ alkenyl, optionally substituted C(O)NC₂-C₈ alkynyl, optionally substituted P(O)(OH)₂, optionally substituted P(O)(OC₁-C₈ alkyl)₂, and optionally substituted P(O)(OC₁-C₈ aryl)₂.

In another embodiment, the degradation tag is a moiety of FORMULAE 6B, 6C, and 6D:

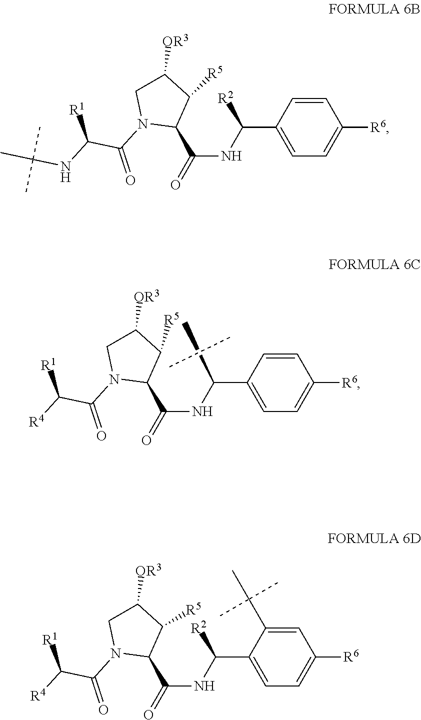

wherein

R¹ and R² are independently selected from hydrogen, hydroxyl, amino, cyano, nitro, optionally substituted C₁-C₈ alkyl, optionally substituted C₂-C₈ alkenyl, optionally substituted C₂-C₈ alkynyl; optionally substituted C₁-C₈alkoxyC₁-C₈alkyl, optionally substituted C₁-C₈ haloalkyl, optionally substituted C₁-C₈ hydroxyalkyl, optionally substituted C₁-C₈ aminoalkyl, optionally substituted C₁-C₈alkylaminoC₁-C₈alkyl, optionally substituted 3-10 membered carbocyclyl, and optionally substituted 4-10 membered heterocyclyl;

R³ is selected from hydrogen, optionally substituted C(O)C₁-C₈ alkyl, optionally substituted C(O)C₁-C₈alkoxyC₁-C₈alkyl, optionally substituted C(O)C₁-C₈ haloalkyl, optionally substituted C(O)C₁-C₈ hydroxyalkyl, optionally substituted C(O)C₁-C₈ aminoalkyl, optionally substituted C(O)C₁-C₈alkylaminoC₁-C₈alkyl, optionally substituted C(O)(3-10 membered carbocyclyl), optionally substituted C(O)(4-10 membered heterocyclyl), optionally substituted C(O)C₂-C₈ alkenyl, optionally substituted C(O)C₂-C₈ alkynyl, optionally substituted C(O)OC₁-C₈alkoxyC₁-C₈alkyl, optionally substituted C(O)OC₁-C₈ haloalkyl, optionally substituted C(O)OC₁-C₈ hydroxyalkyl, optionally substituted C(O)OC₁-C₈ aminoalkyl, optionally substituted C(O)OC₁-C₈alkylaminoC₁-C₈alkyl, optionally substituted C(O)O(3-10 membered carbocyclyl), optionally substituted C(O)O(4-10 membered heterocyclyl), optionally substituted C(O)OC₂-C₈ alkenyl, optionally substituted C(O)OC₂-C₈ alkynyl, optionally substituted C(O)NC₁-C₈alkoxyC₁-C₈alkyl, optionally substituted C(O)NC₁-C₈ haloalkyl, optionally substituted C(O)NC₁-C₈ hydroxyalkyl, optionally substituted C(O)NC₁-C₈ aminoalkyl, optionally substituted C(O)NC₁-C₈alkylaminoC₁-C₈alkyl, optionally substituted C(O)N(3-10 membered carbocyclyl), optionally substituted C(O)N(4-10 membered heterocyclyl), optionally substituted C(O)NC₂-C₈ alkenyl, optionally substituted C(O)NC₂-C₈ alkynyl, optionally substituted P(O)(OH)₂, optionally substituted P(O)(OC₁-C₈ alkyl)₂, and optionally substituted P(O)(OC₁-C₈ aryl)₂, and

R⁴ is selected from NR⁷R⁸,

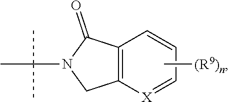

optionally substituted C₁-C₈alkoxy, optionally substituted 3-10 membered carbocyclyl, optionally substituted 4-10 membered heterocyclyl, optionally substituted aryl, and optionally substituted heteraryl, in which

R⁷ is selected from hydrogen, optionally substituted C₁-C₈alkyl, optionally substituted C₁-C₈cycloalkyl, optionally substituted C₁-C₈alkyl-CO, optionally substituted C₁-C₈cycloalkyl-CO, optionally substituted C₁-C₈cycloalkyl-C₁-C₈alkyl-CO, optionally substituted 4-10 membered heterocyclyl-CO, optionally substituted 4-10 membered heterocyclyl-C₁-C₈alkyl-CO, optionally substituted aryl-CO, optionally substituted aryl-C₁-C₈alkyl-CO, optionally substituted heteroaryl-CO, optionally substituted heteroaryl-C₁-C₈alkyl-CO, optionally substituted aryl, and optionally substituted heteroaryl;

R⁸ is selected from hydrogen, optionally substituted C₁-C₈alkyl, and optionally substituted C₁-C₈cycloalkyl;

R⁹, at each occurrence, is independently selected from hydrogen, halogen, cyano, optionally substituted C₁-C₈alkyl, optionally substituted C₁-C₈cycloalkyl, optionally substituted C₁-C₈heterocycloalkyl, optionally substituted C₁-C₈alkoxy, optionally substituted C₁-C₈cycloalkoxy, halo substituted C₁-C₈alkyl, halo substituted C₁-C₈cycloalkyl, halo substituted C₁-C₈alkoxl, halo substituted C₁-C₈cycloalkoxy, and halo substituted C₁-C₈heterocycloalkyl;

X is selected from CH and N; and

n is 0, 1, 2, 3, or 4;

R⁶ is selected from hydrogen, halogen, hydroxyl, amino, cyano, nitro, optionally substituted C₁-C₈alkyl, optionally substituted C₁-C₈cycloalkyl, optionally substituted C₁-C₈alkoxy, and optionally substituted C₁-C₈cycloalkoxy, optionally substituted C₁-C₈heterocycloalkyl, optionally substituted aryl, and optionally substituted heteroaryl, preferably, halogen, cyano, optionally substituted imidazole, optionally substituted pyrazole, optionally substituted oxadiazole, optionally substituted triazole, 4-methylthiazol-5-yl, or oxazol-5-yl group.

In another embodiment, the degradation tag is a moiety of FORMULA 7A:

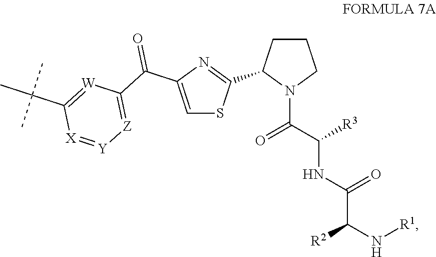

wherein

V, W, X, and Z are independently selected from CR⁴ and N; and

R¹, R², R³, and R⁴ are independently selected from hydrogen, halogen, cyano, nitro, optionally substituted C₁-C₈ alkyl, optionally substituted C₂-C₈ alkenyl, and optionally substituted C₂-C₈ alkynyl; optionally substituted C₁-C₈alkoxyC₁-C₈alkyl, optionally substituted C₁-C₈ haloalkyl, optionally substituted C₁-C₈ hydroxyalkyl, optionally substituted C₁-C₈alkoxy, optionally substituted C₁-C₈alkylamino, optionally substituted 3-10 membered carbocyclyl, and optionally substituted 4-10 membered heterocyclyl.

In another embodiment, the degradation tag is a moiety of FORMULA 7B:

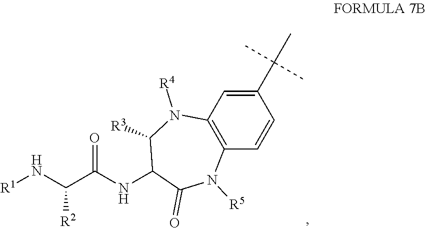

wherein

R¹, R², and R³ are independently selected from hydrogen, halogen, optionally substituted C₁-C₈ alkyl, optionally substituted C₁-C₈alkoxyC₁-C₈alkyl, optionally substituted C₁-C₈ haloalkyl, optionally substituted C₁-C₈ hydroxyalkyl, optionally substituted C₃-C₇ cycloalkyl, optionally substituted 3-7 membered heterocyclyl, optionally substituted C₂-C₈ alkenyl, and optionally substituted C₂-C₈ alkynyl;

R⁴ and R⁵ are independently selected from hydrogen, COR⁶, CO₂R⁶, CONR⁶R⁷, SOR⁶, SO₂R⁶, SO₂NR⁶R⁷, optionally substituted C₁-C₈ alkyl, optionally substituted C₁-C₈alkoxyC₁-C₈alkyl, optionally substituted C₁-C₈alkylaminoC₁-C₈alkyl, optionally substituted aryl-C₁-C₈alkyl, optionally substituted 3-8 membered cycloalkyl, optionally substituted 3-8 membered heterocyclyl, optionally substituted aryl, and optionally substituted heteroaryl, wherein

R⁶ and R⁷ are independently selected from hydrogen, optionally substituted C₁-C₈ alkyl, optionally substituted C₁-C₈alkoxyC₁-C₈alkyl, optionally substituted C₁-C₈alkylaminoC₁-C₈alkyl, optionally substituted 3-8 membered cycloalkyl, optionally substituted 3-8 membered heterocyclyl, optionally substituted aryl, and optionally substituted heteroaryl, or

R⁶ and R⁷ together with the atom to which they are connected form a 4-8 membered cycloalkyl or heterocyclyl ring.

In another embodiment, the degradation tag is a moiety of FORMULA 5A, 5B, 5C, 5D, 5E, 5F, 5G, 5H, and 5I.

In another embodiment, the degradation tag is a moiety of FORMULA 5A, 5B, 5C, 5D, 5E, and 5F.

In another embodiment, the degradation tag is derived from any of the following:

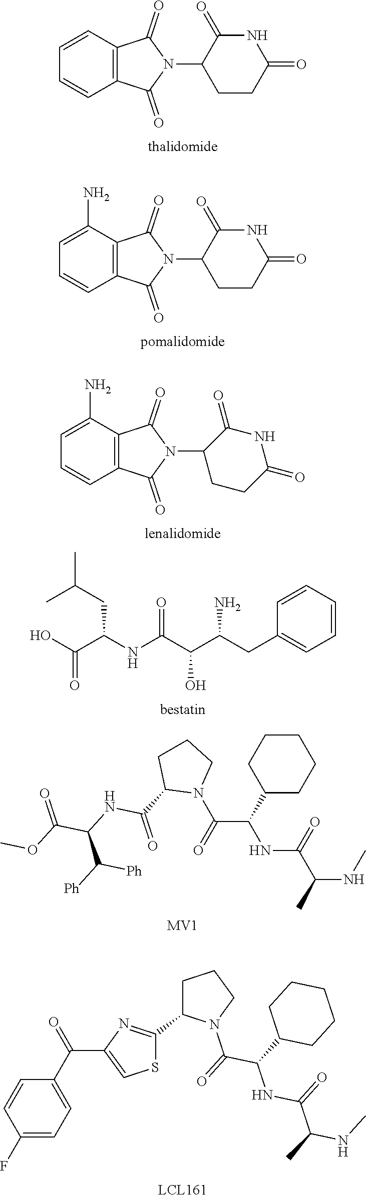

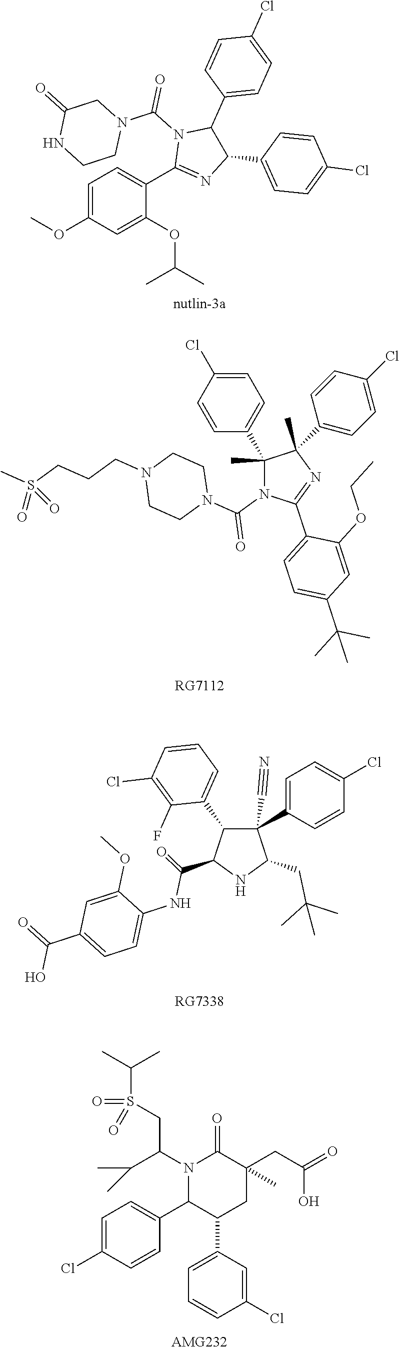

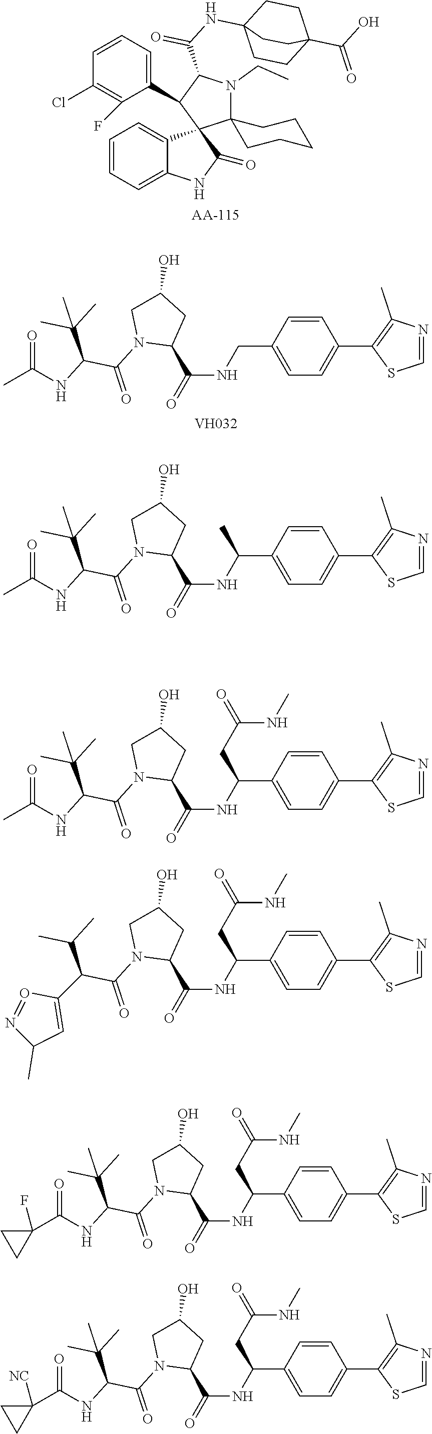

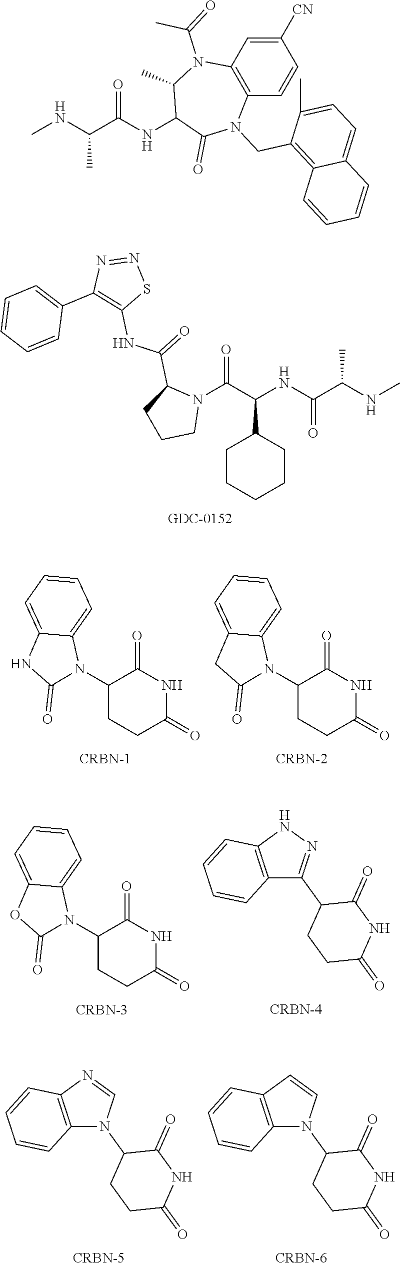

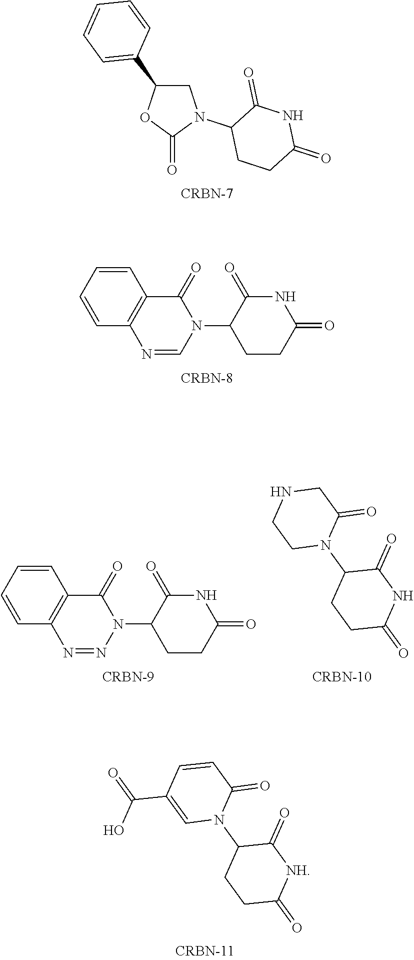 [SMILES: O=C1CCC(N2C[C@H](c3ccccc3)OC2=O)C(=O)N1 ; O=C1CCC(n2cnc3ccccc3c2=O)C(=O)N1 ; O=C1CCC(n2nnc3ccccc3c2=O)C(=O)N1 ; O=C1CCC(N2CCNCC2=O)C(=O)N1 ; O=C1CCC(n2cc(C(=O)O)ccc2=O)C(=O)N1]

In another embodiment, the degradation tag is derived from any of the following: thalidomide, pomalidomide, lenalidomide, CRBN-1, CRBN-2, CRBN-3, CRBN-4, CRBN-5, CRBN-6, CRBN-7, CRBN-8, CRBN-9, CRBN-10, and CRBN-11.

In another embodiment, the degradation tag is selected from the group consisting of:

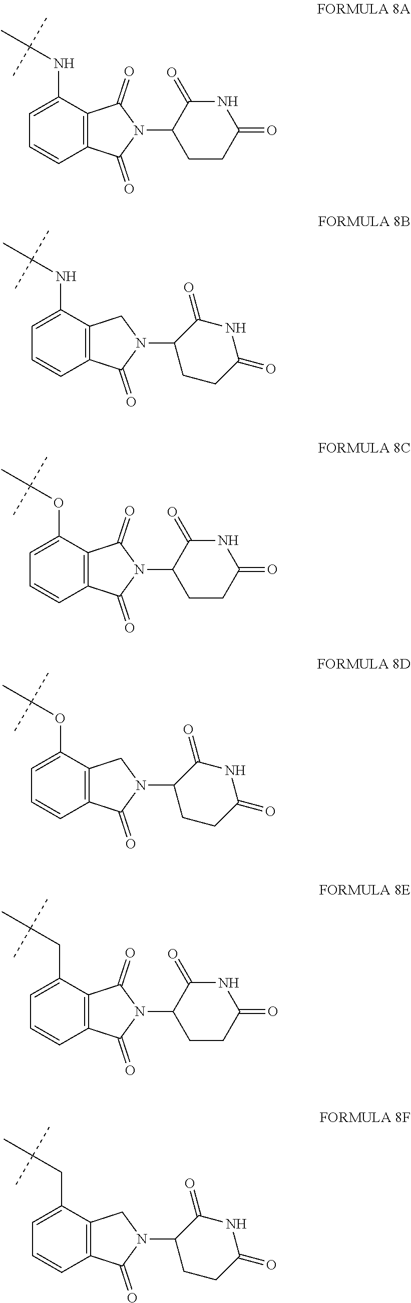

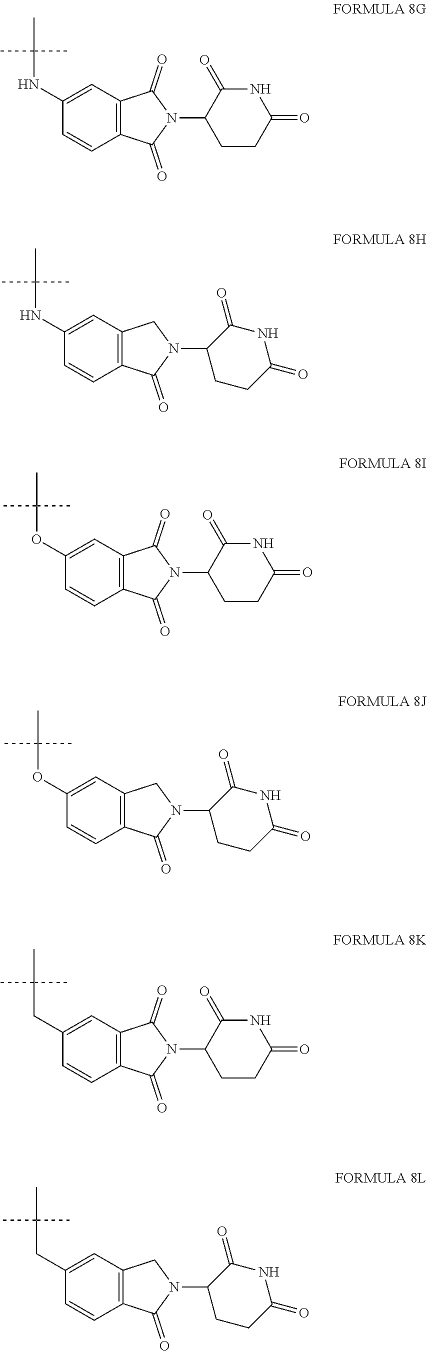

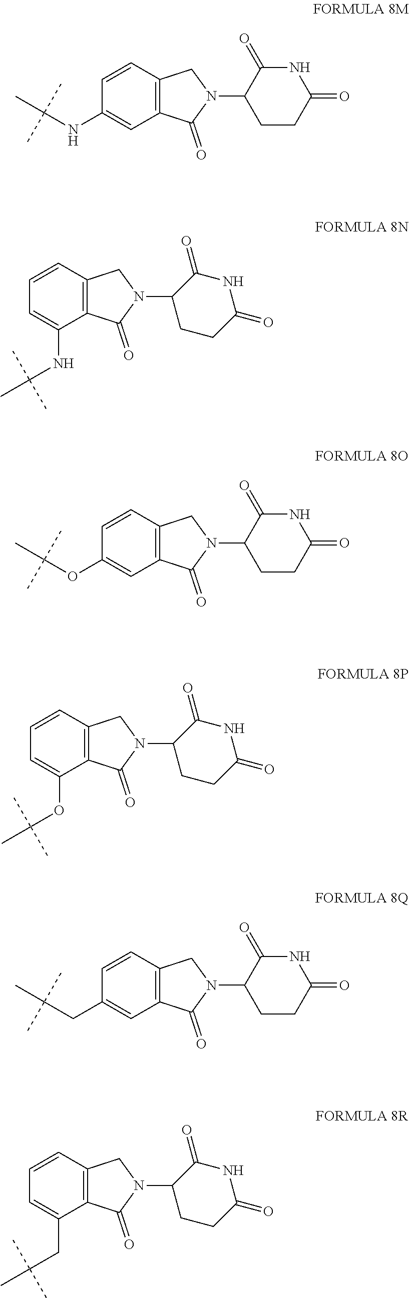

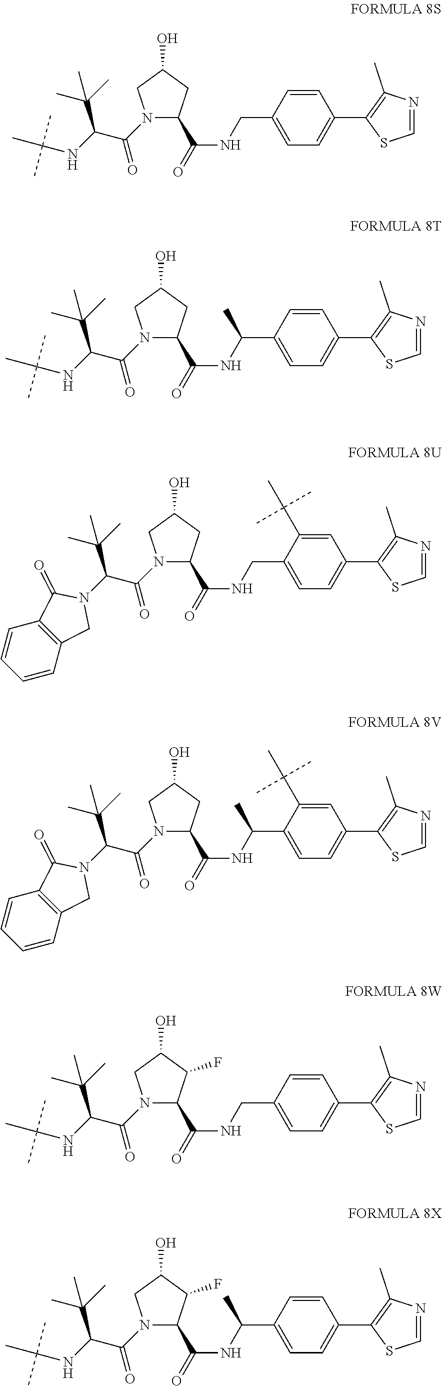

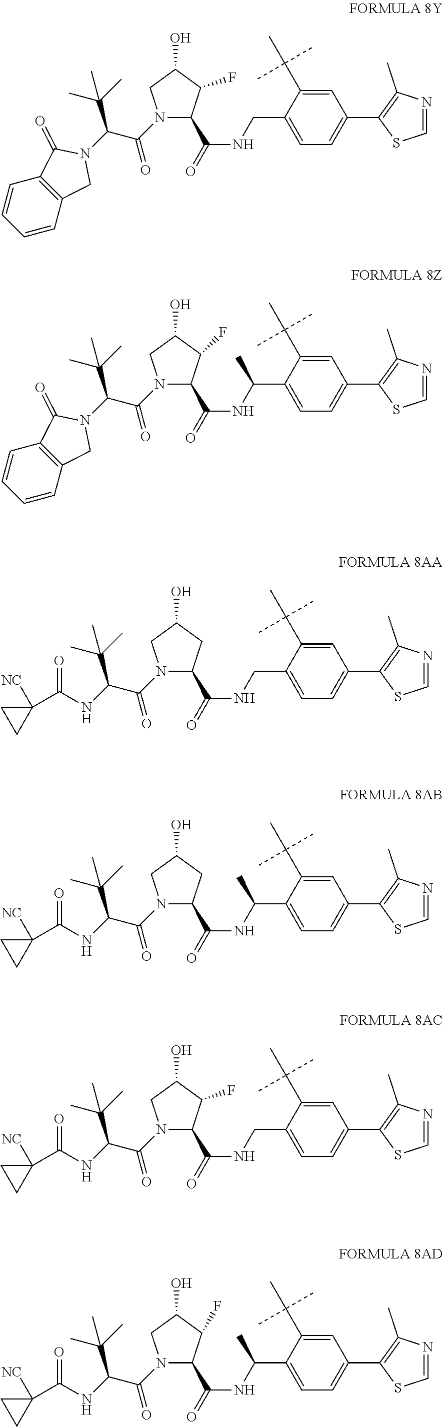

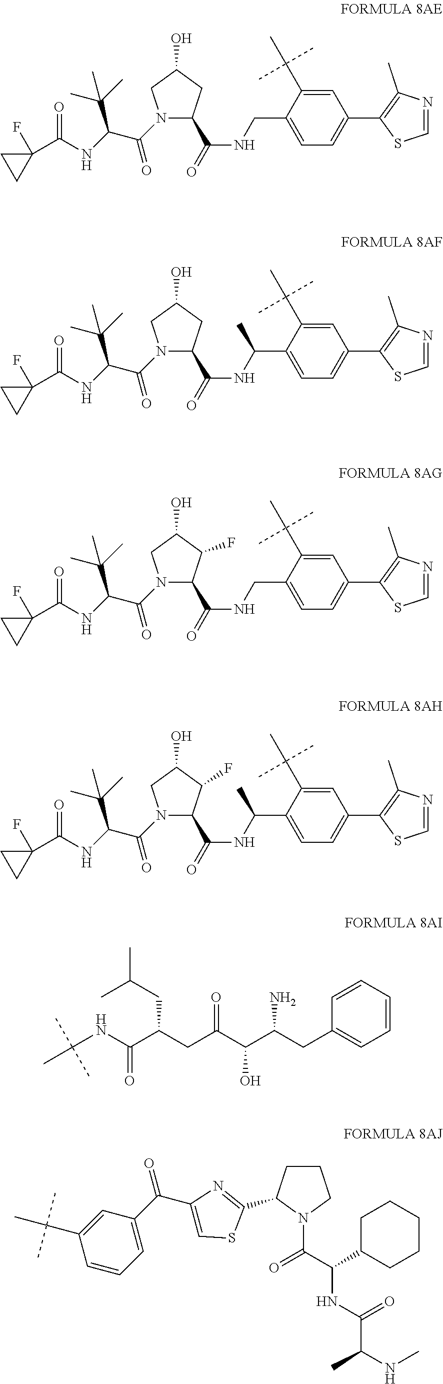

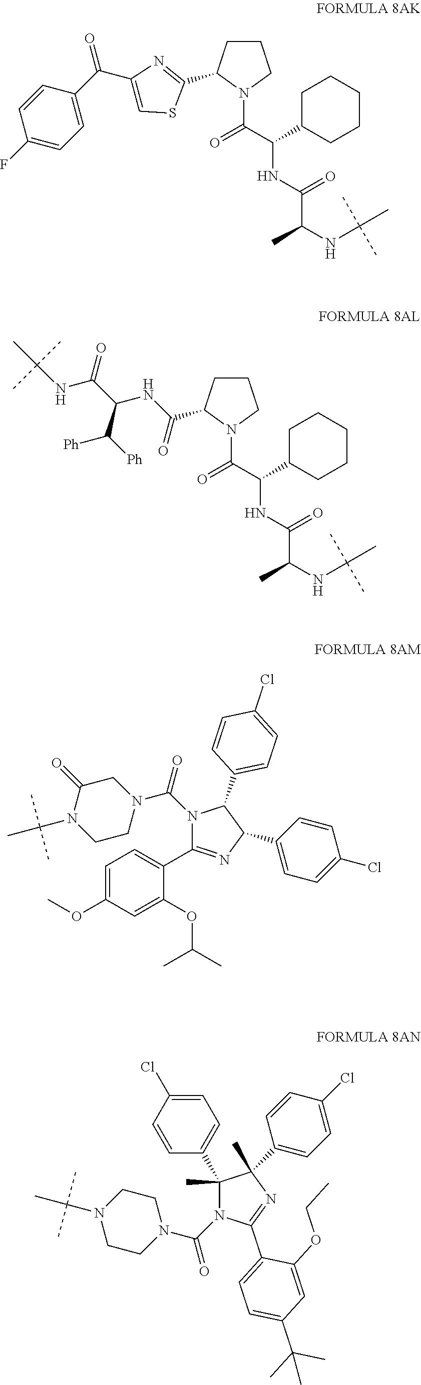

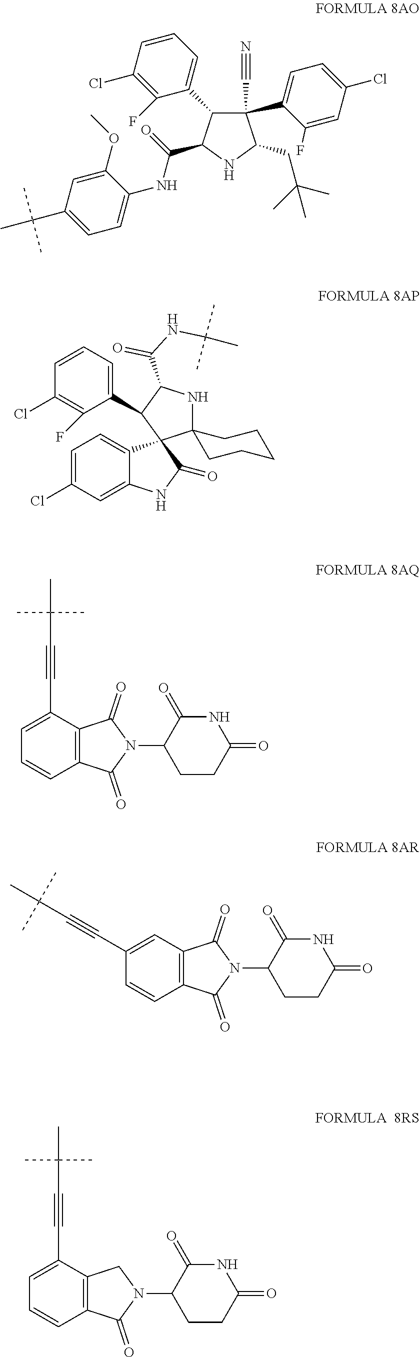

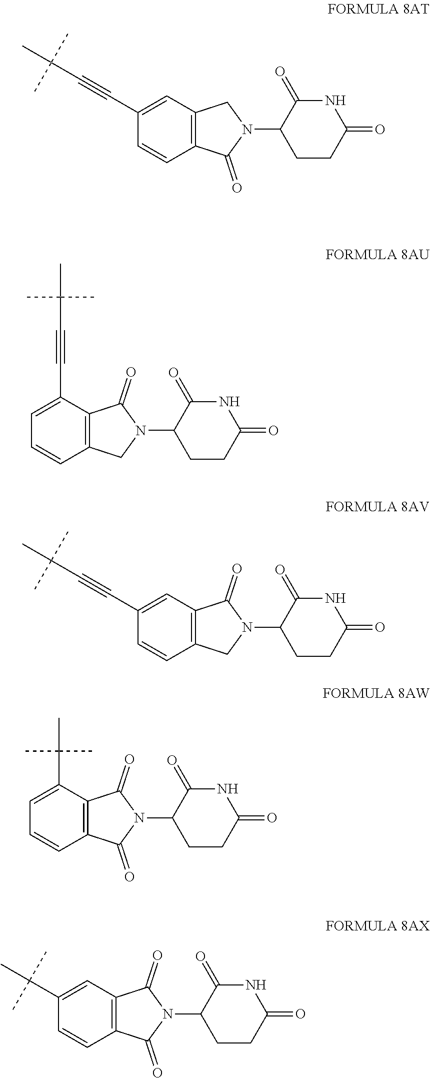

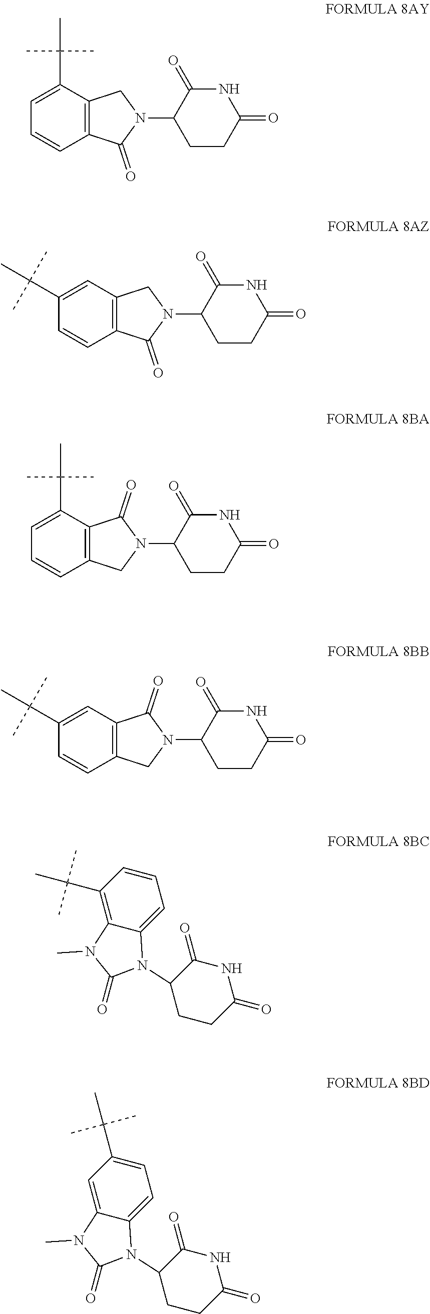

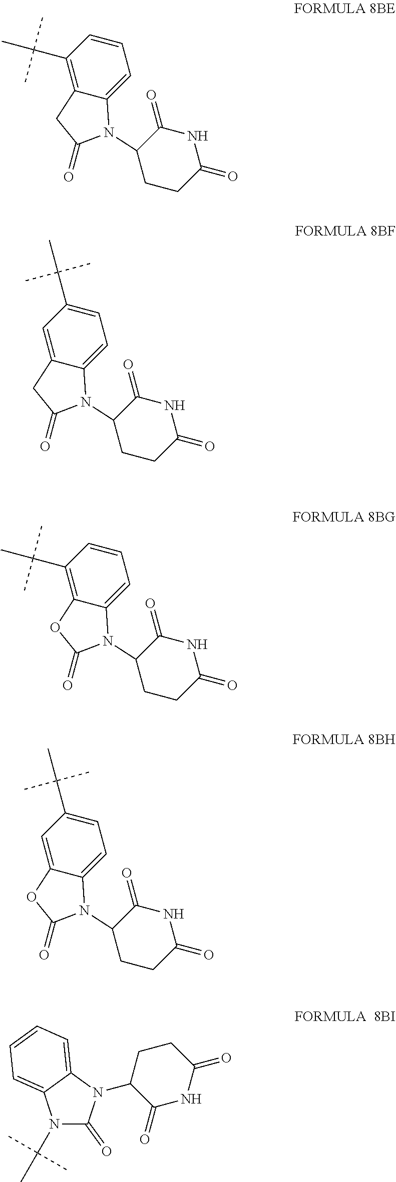

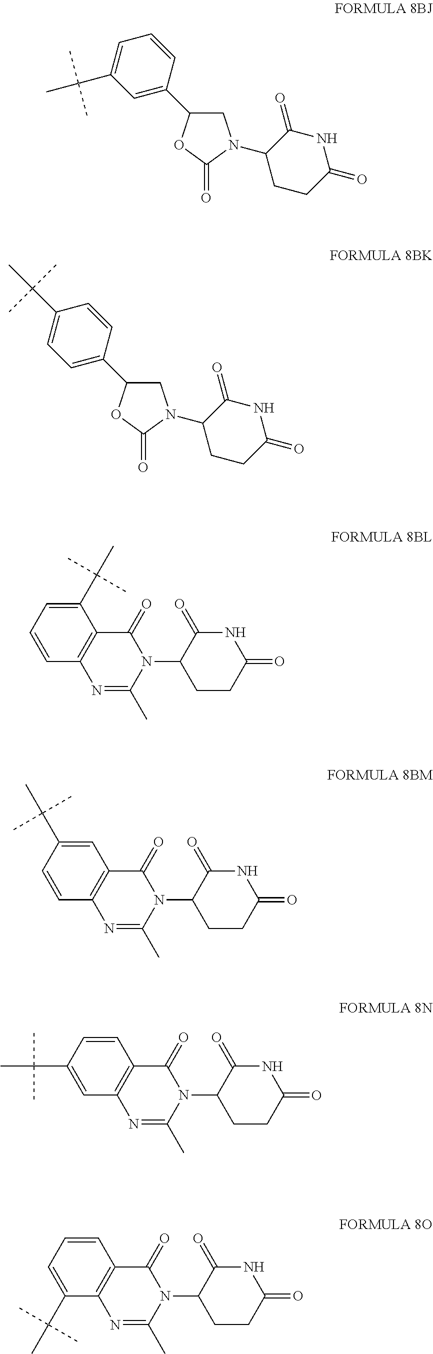

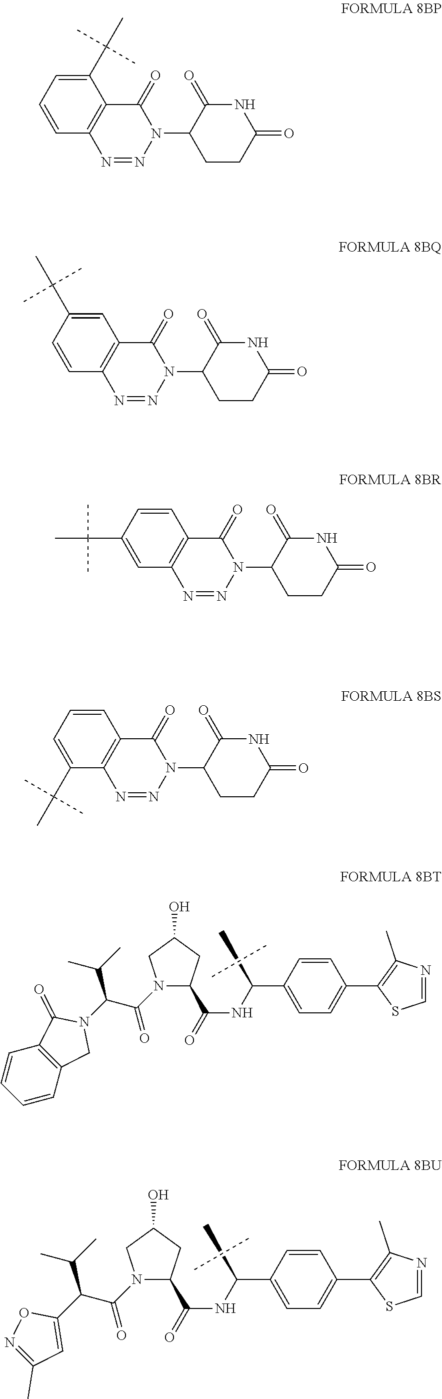

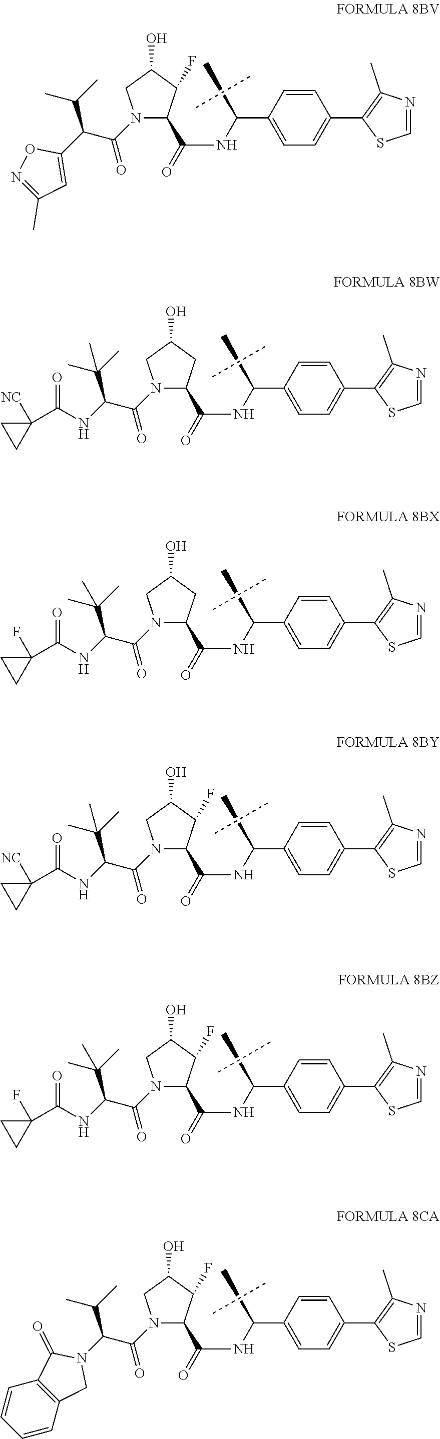

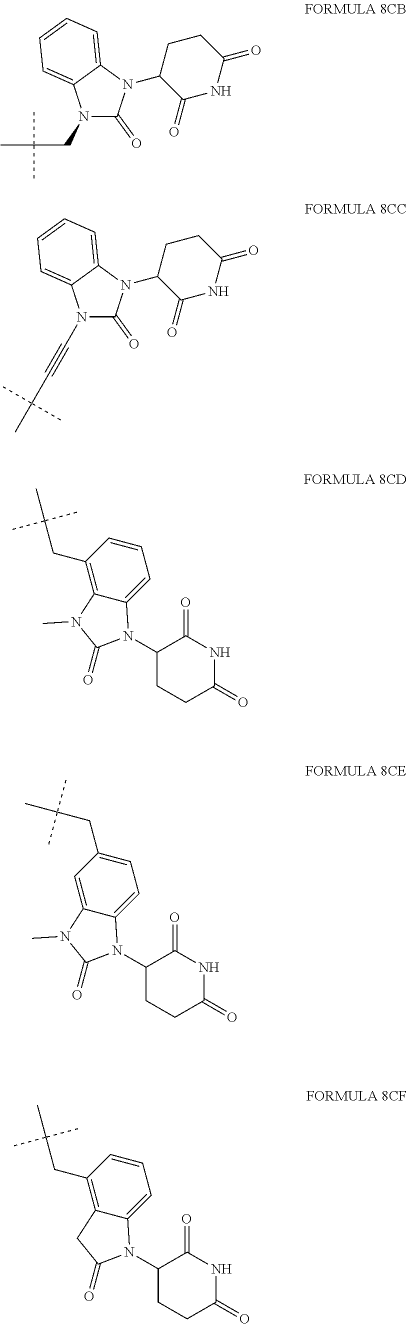

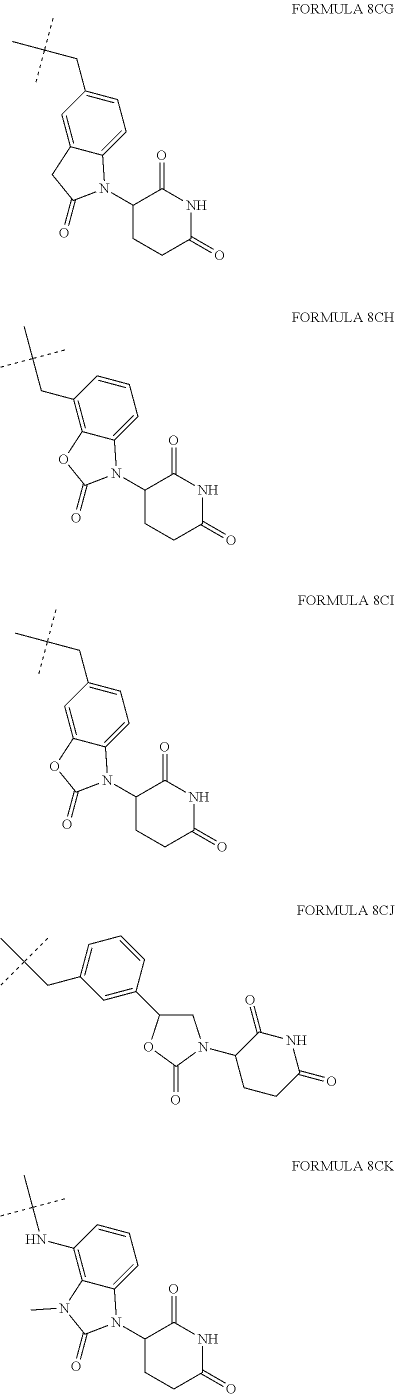

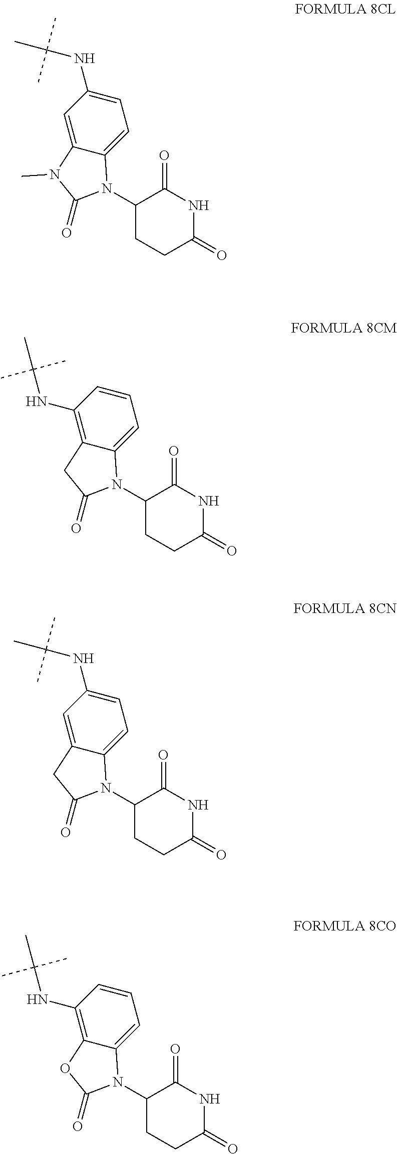

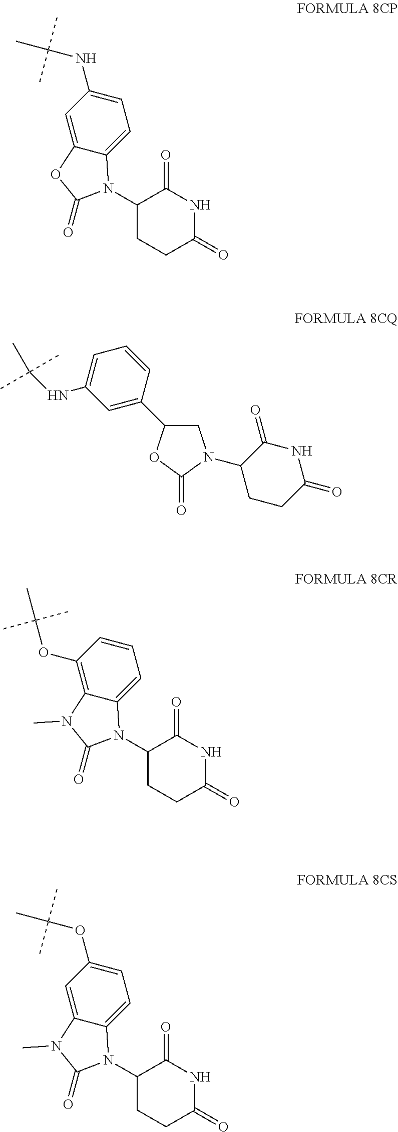

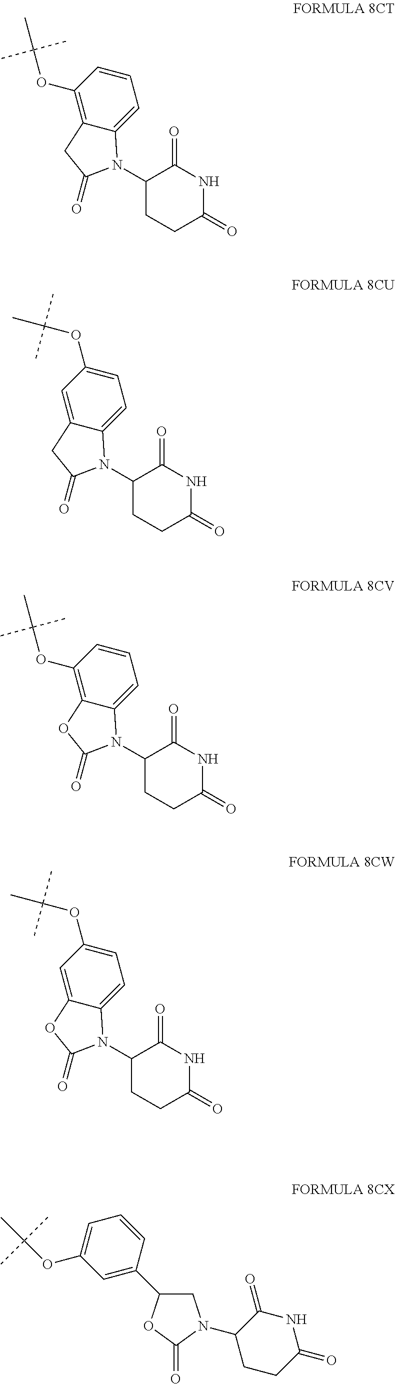

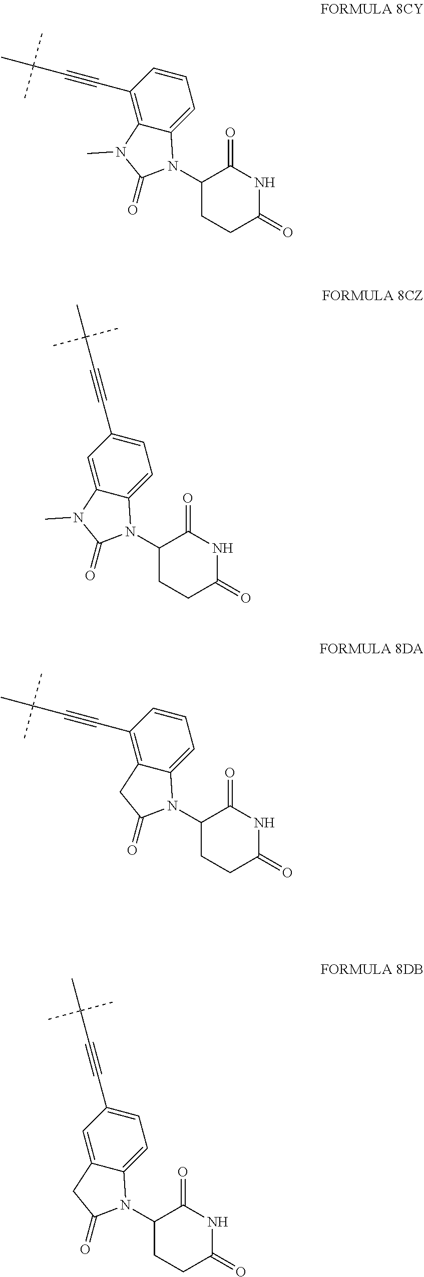

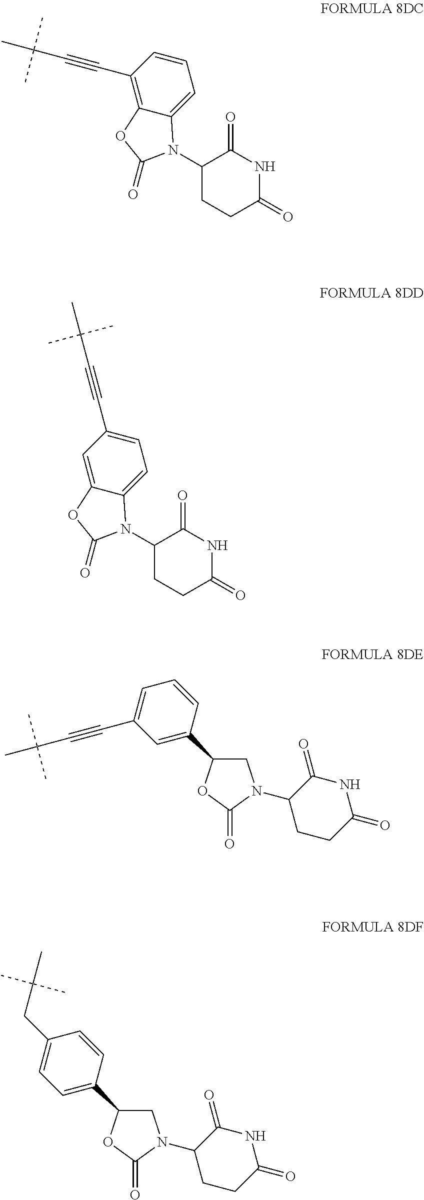

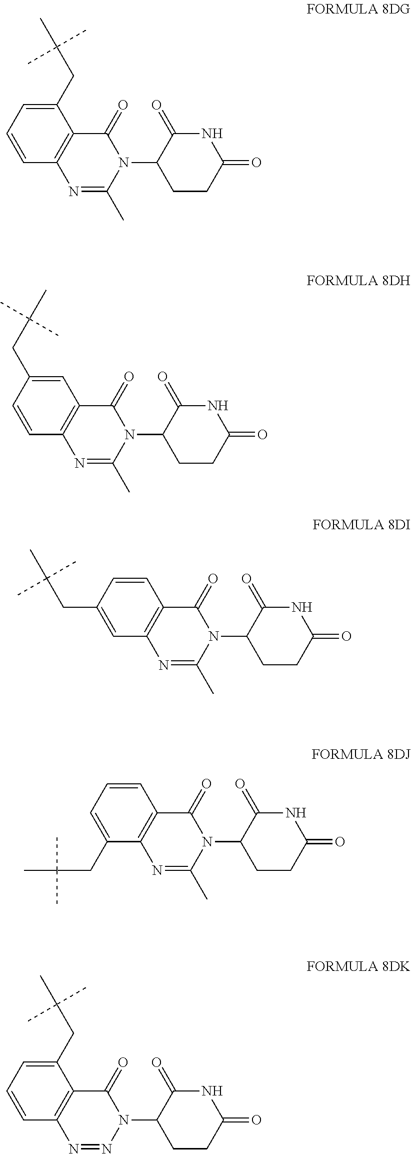

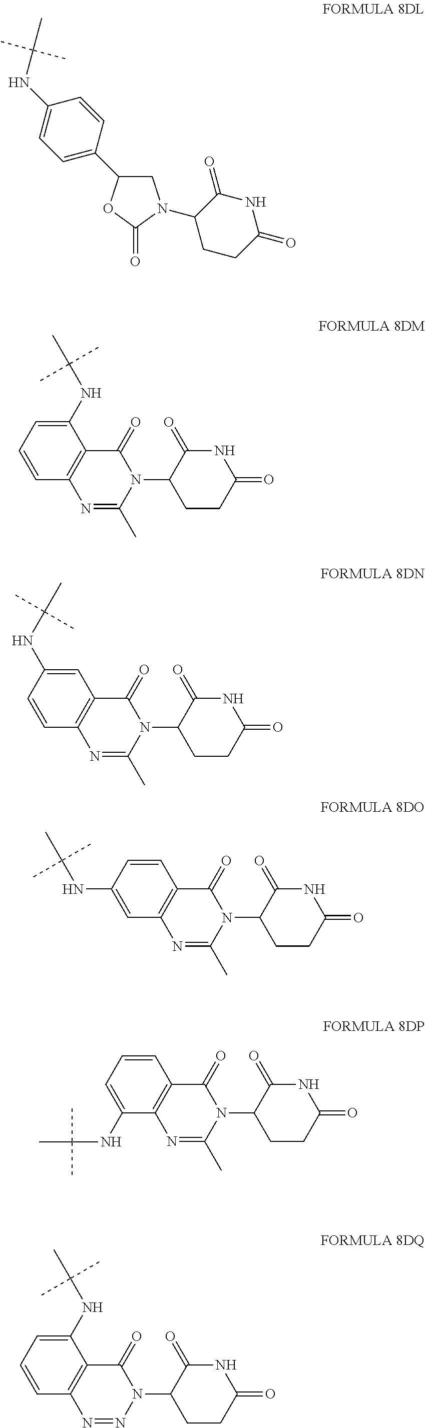

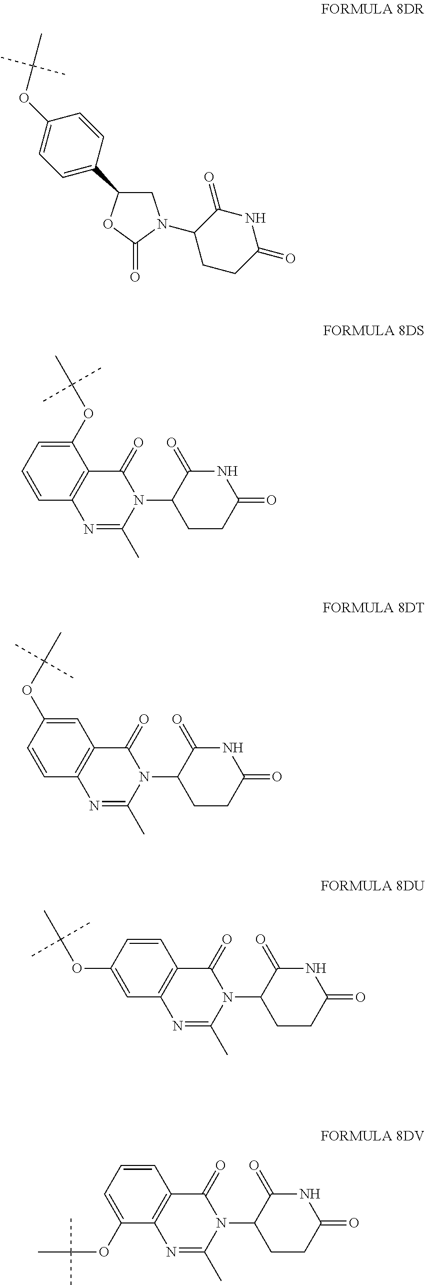

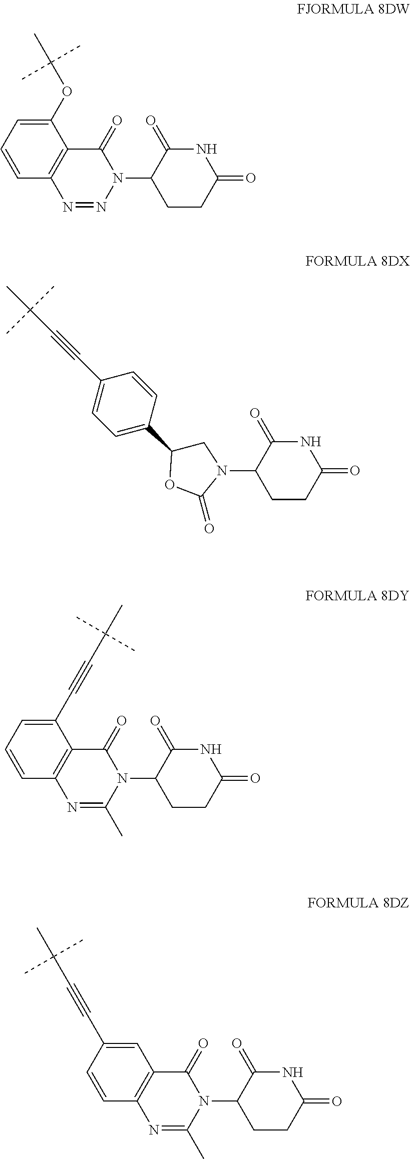

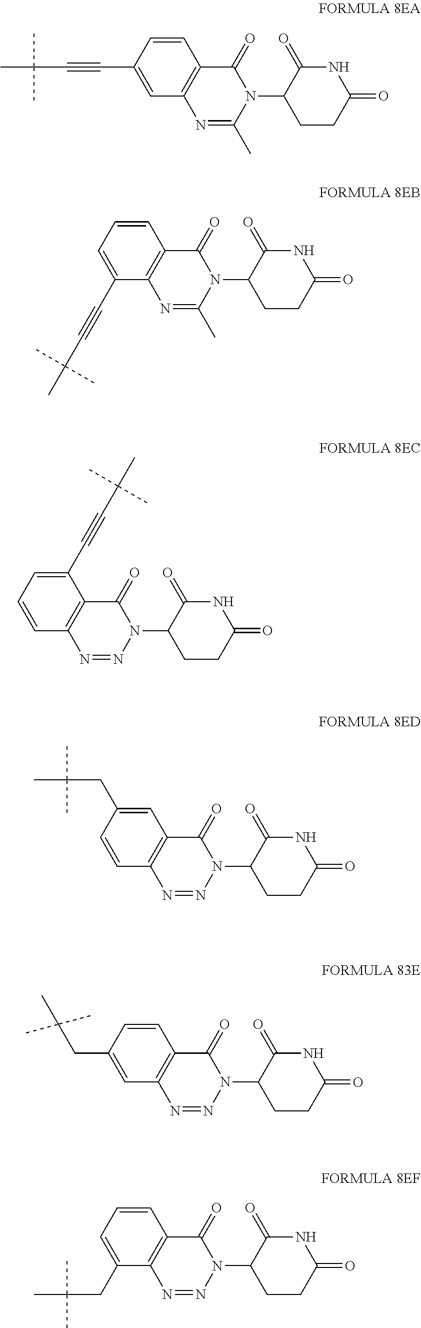

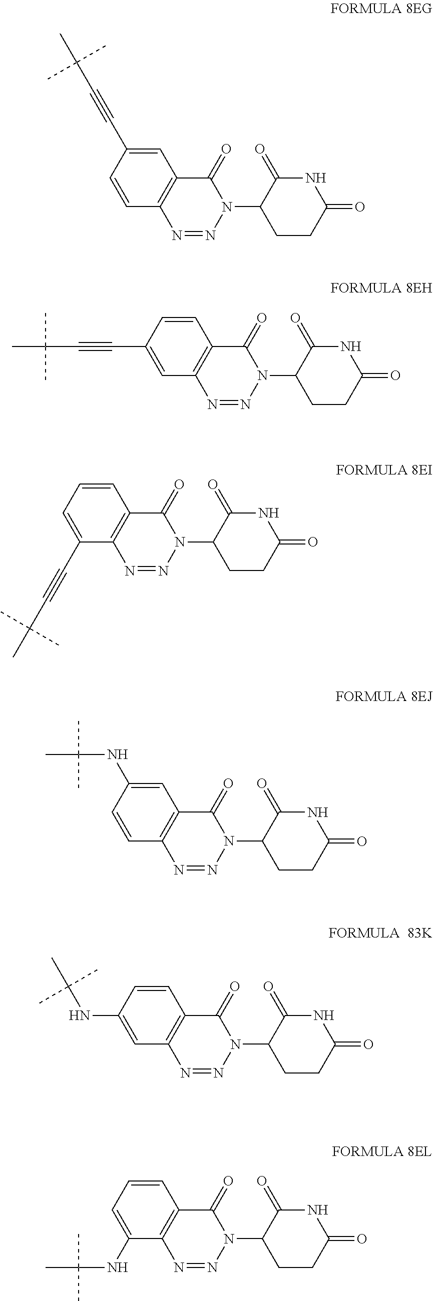

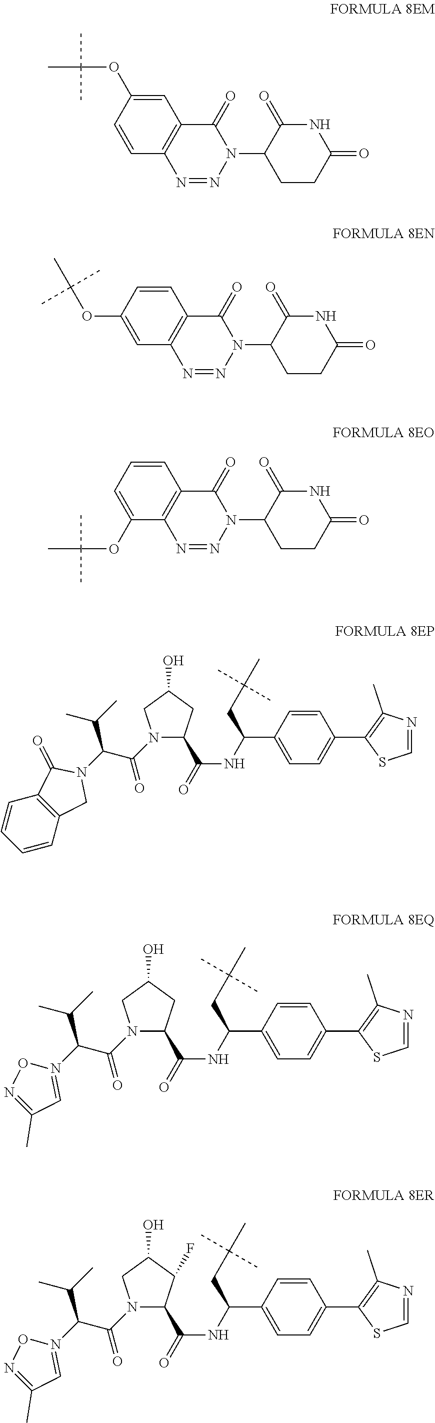

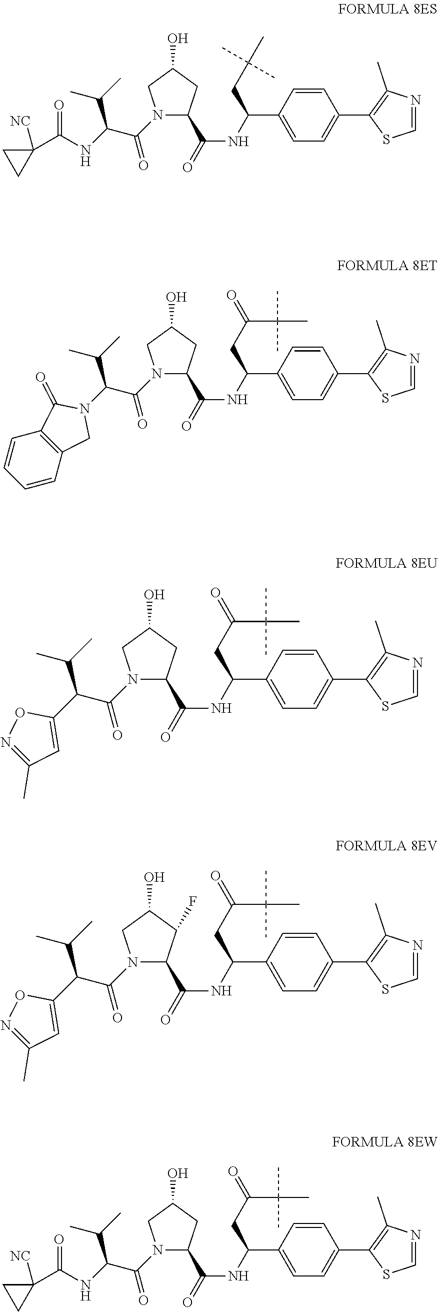

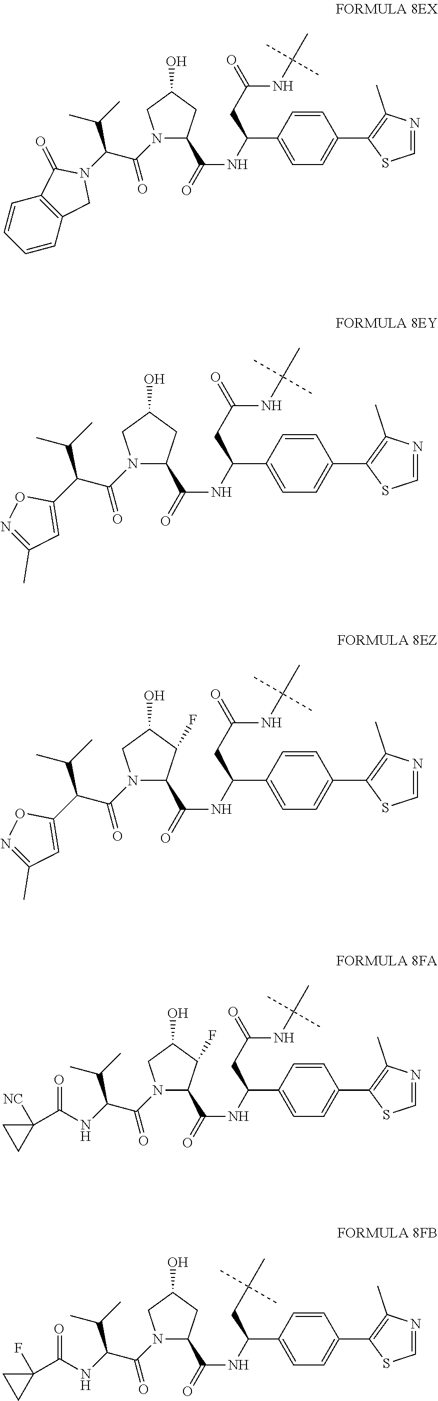

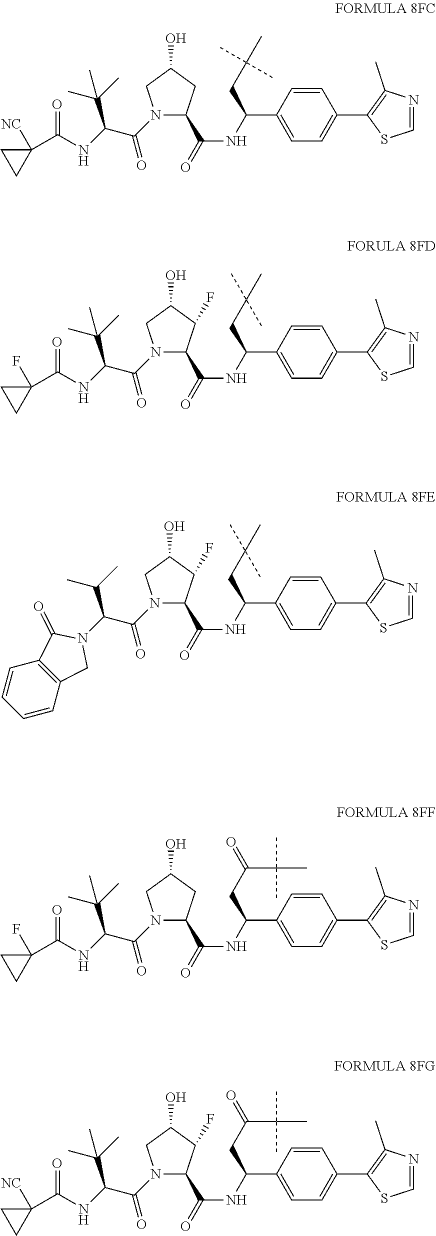

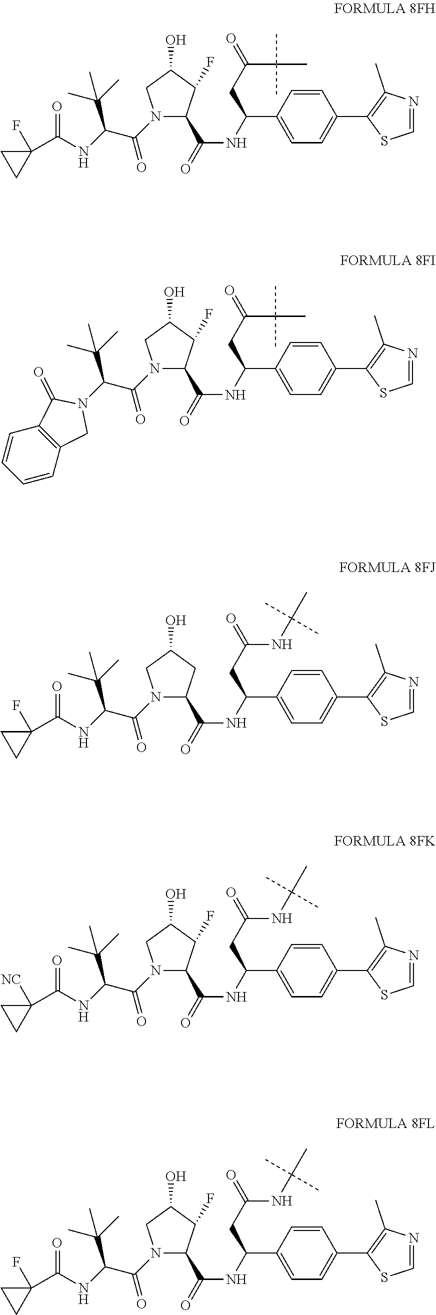

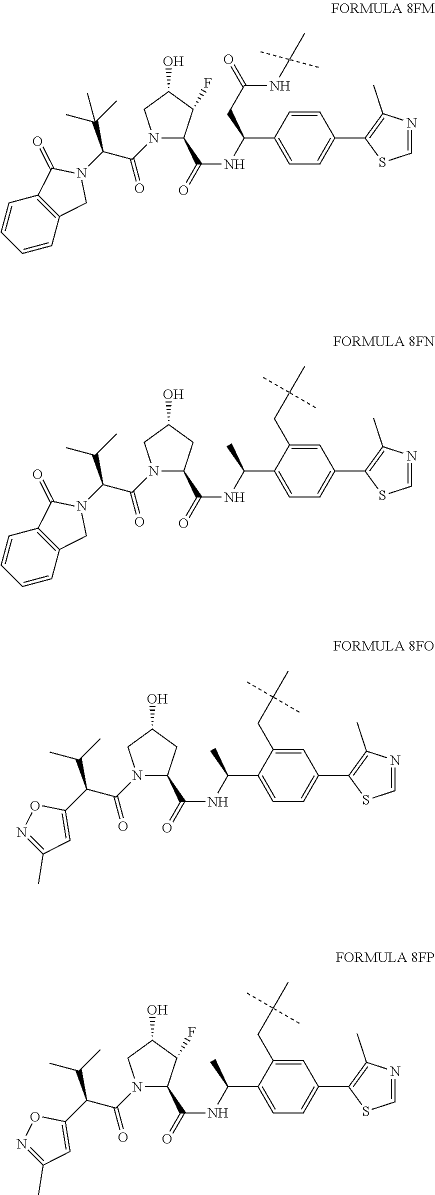

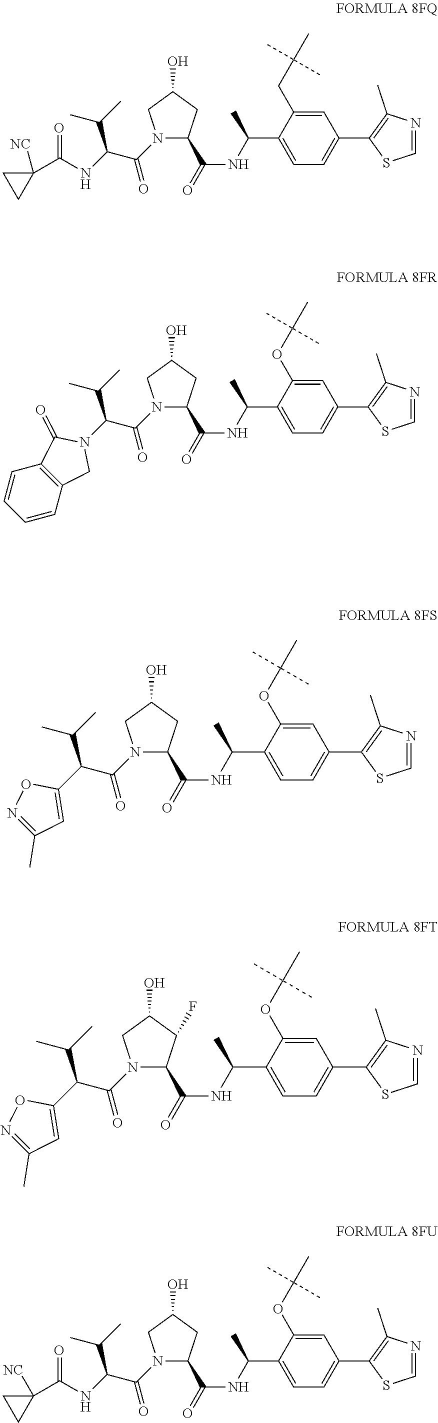

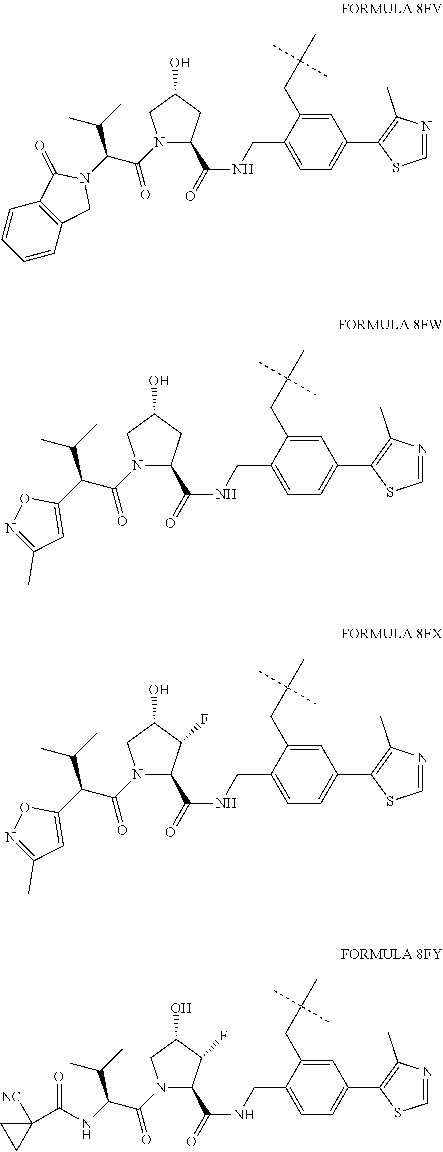

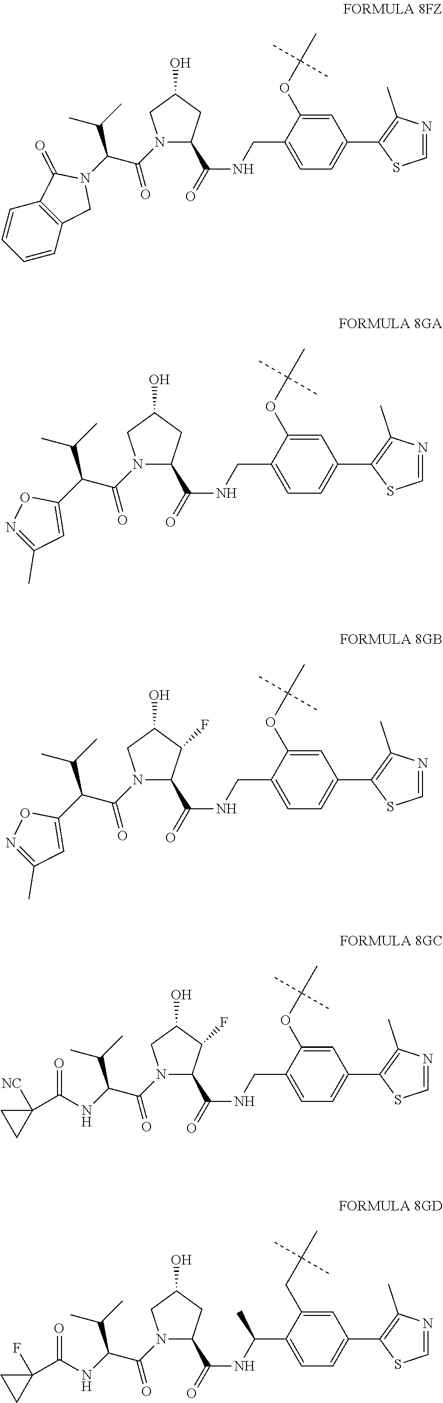

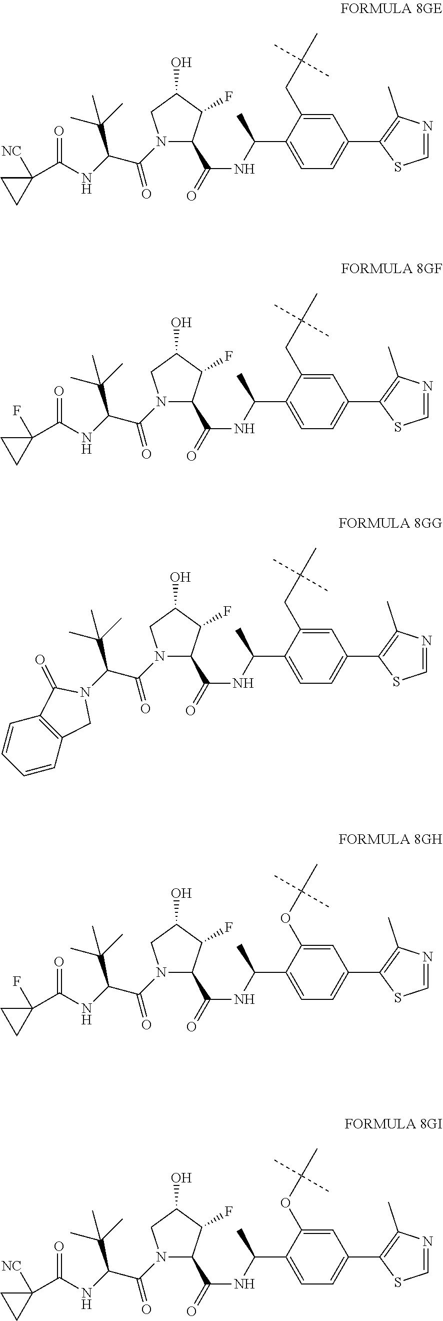

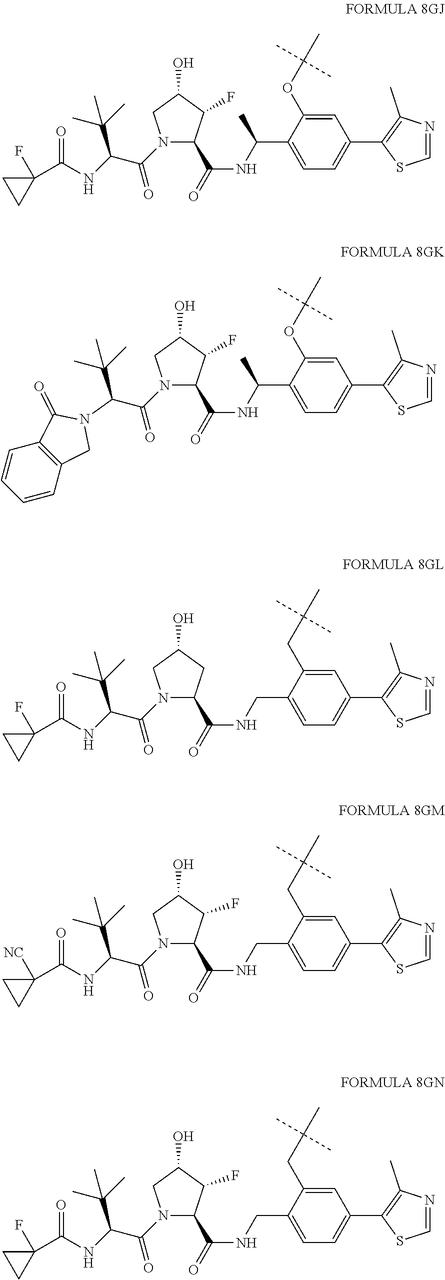

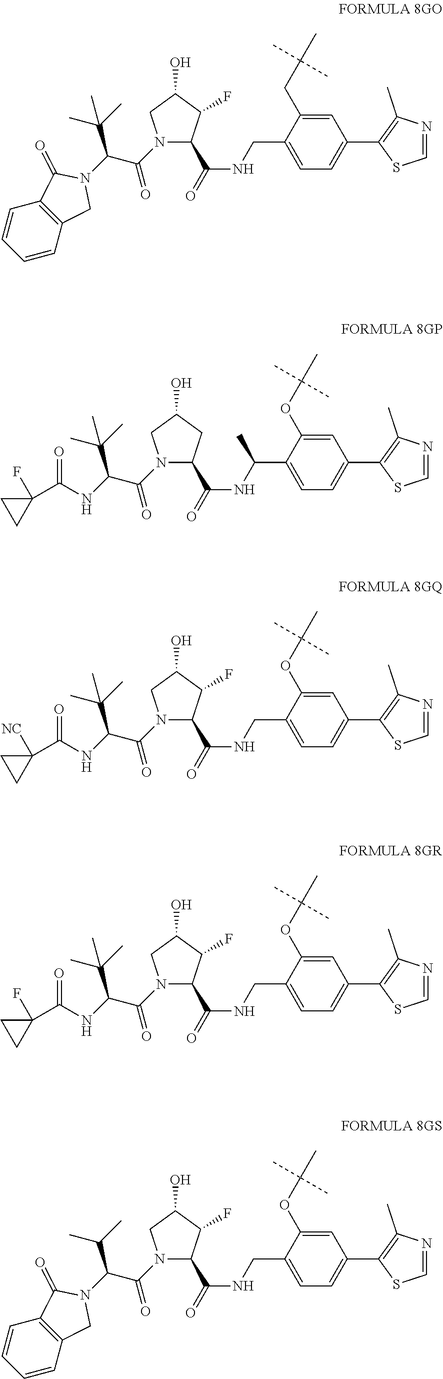

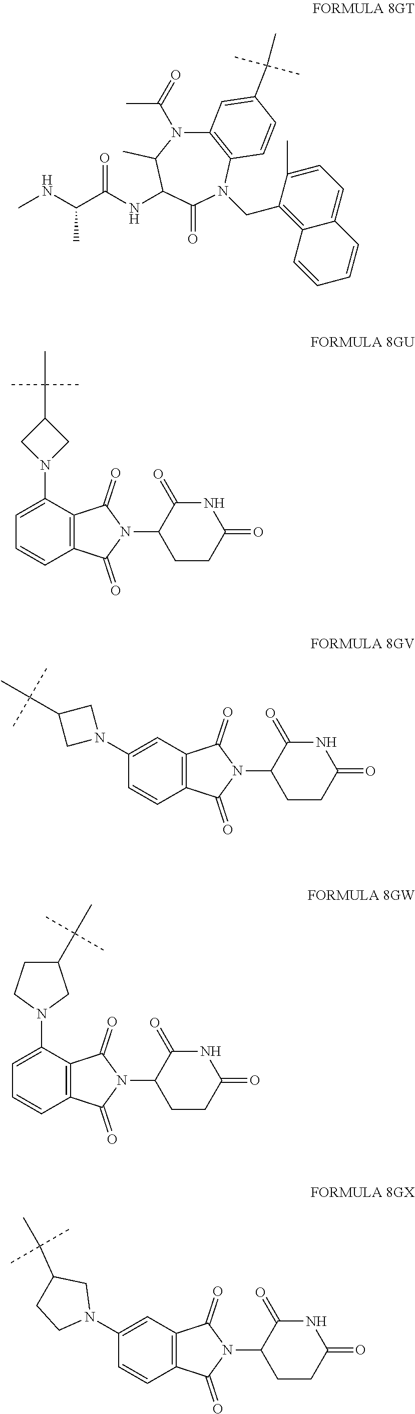

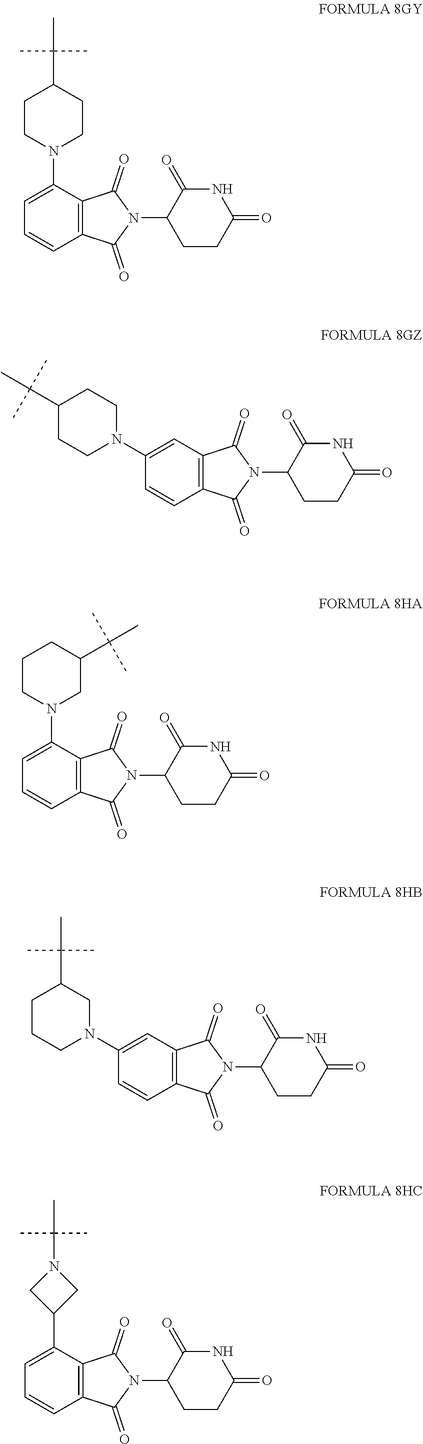

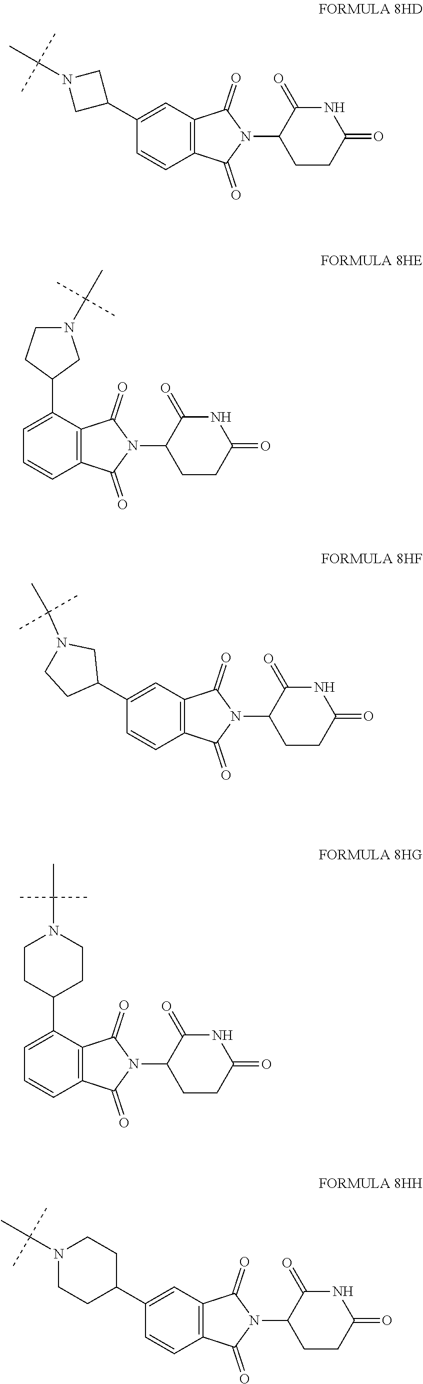

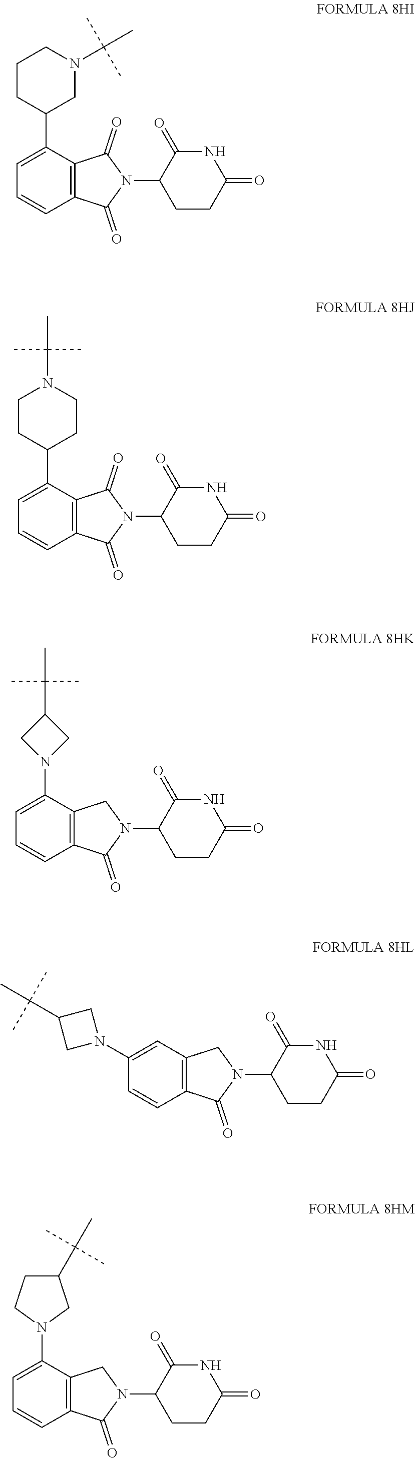

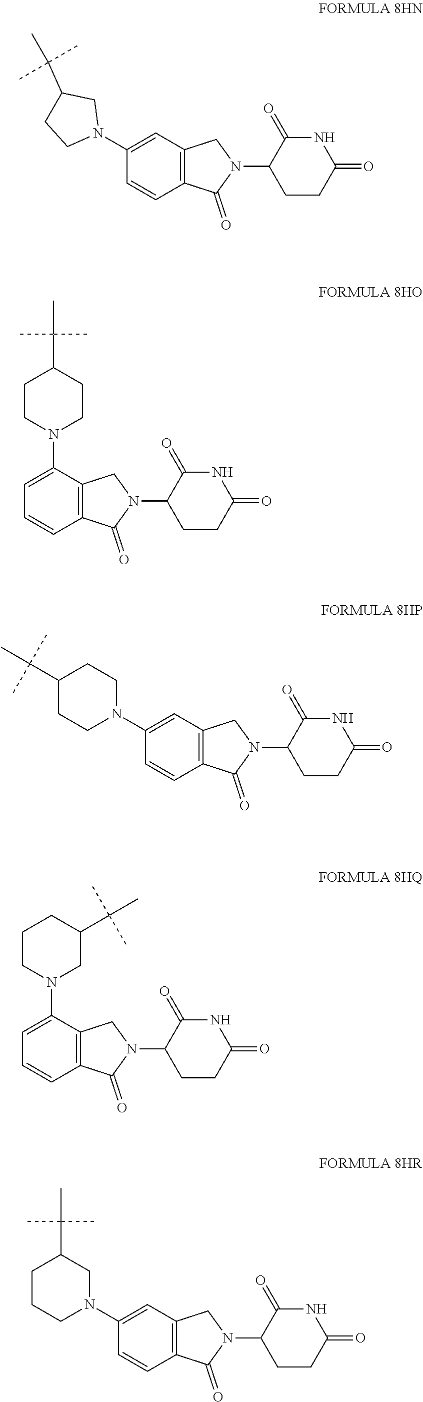

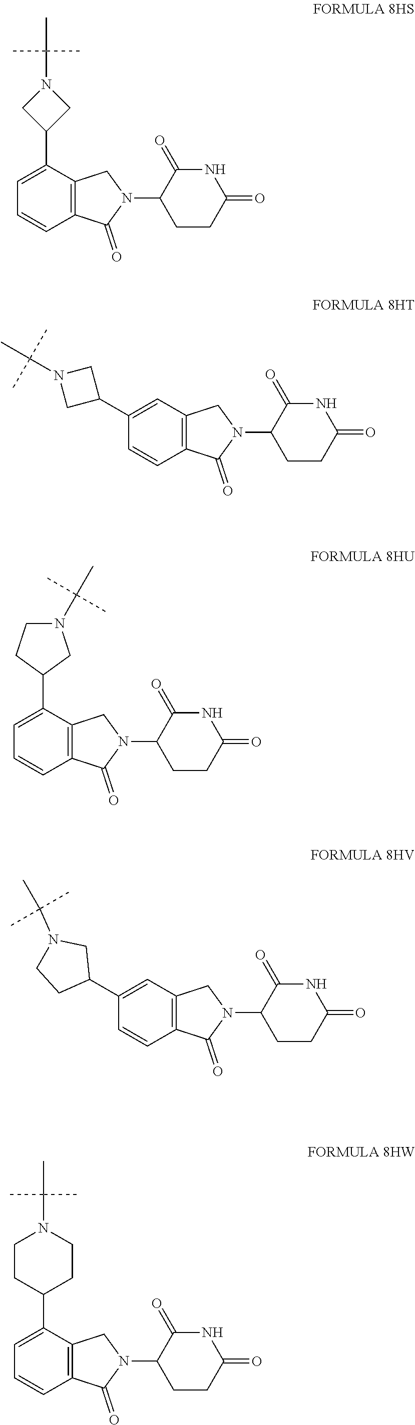

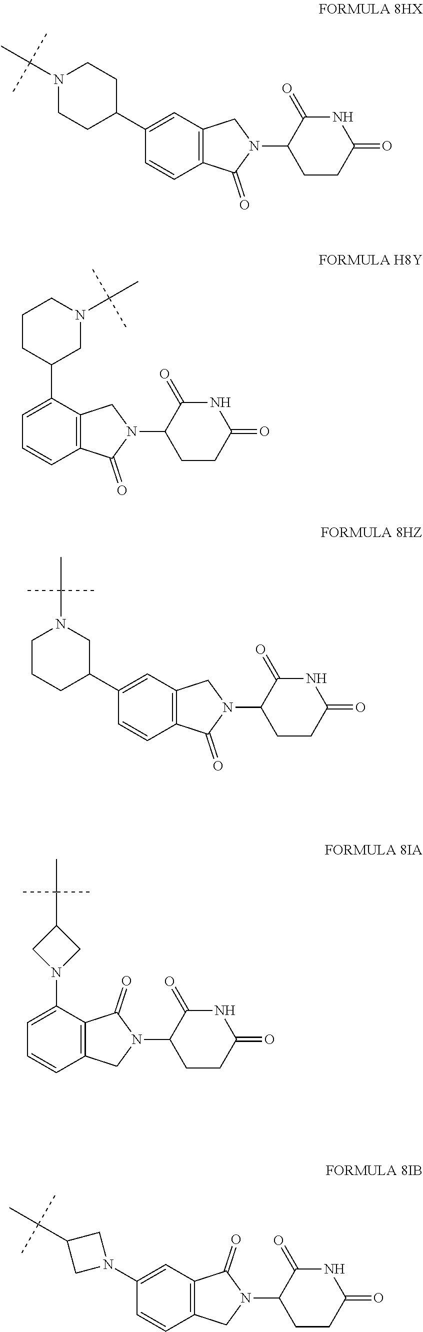

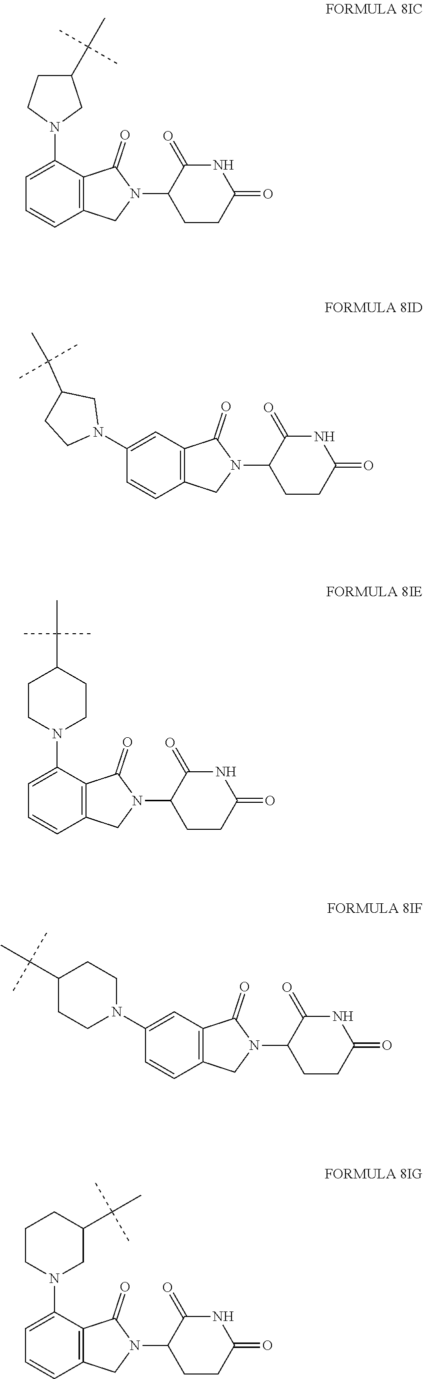

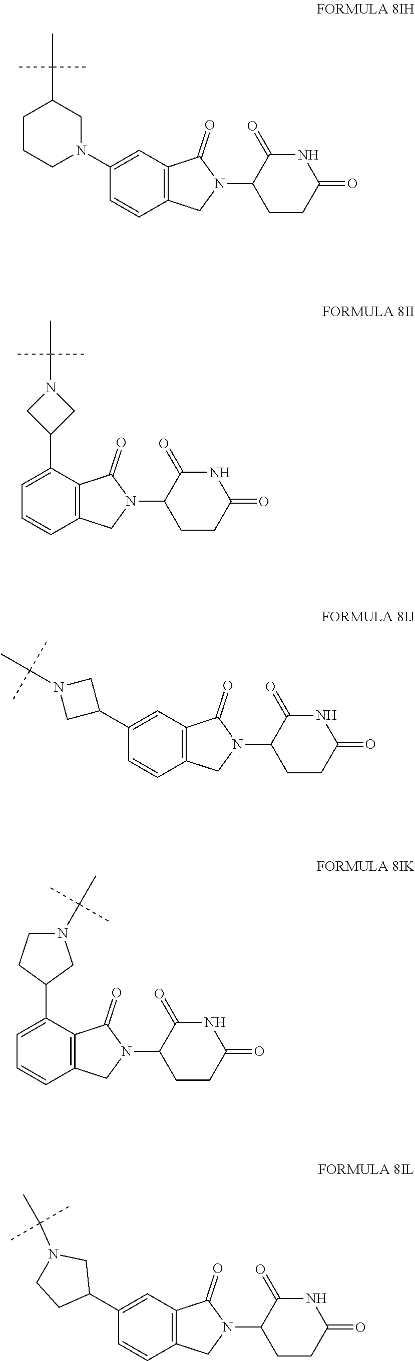

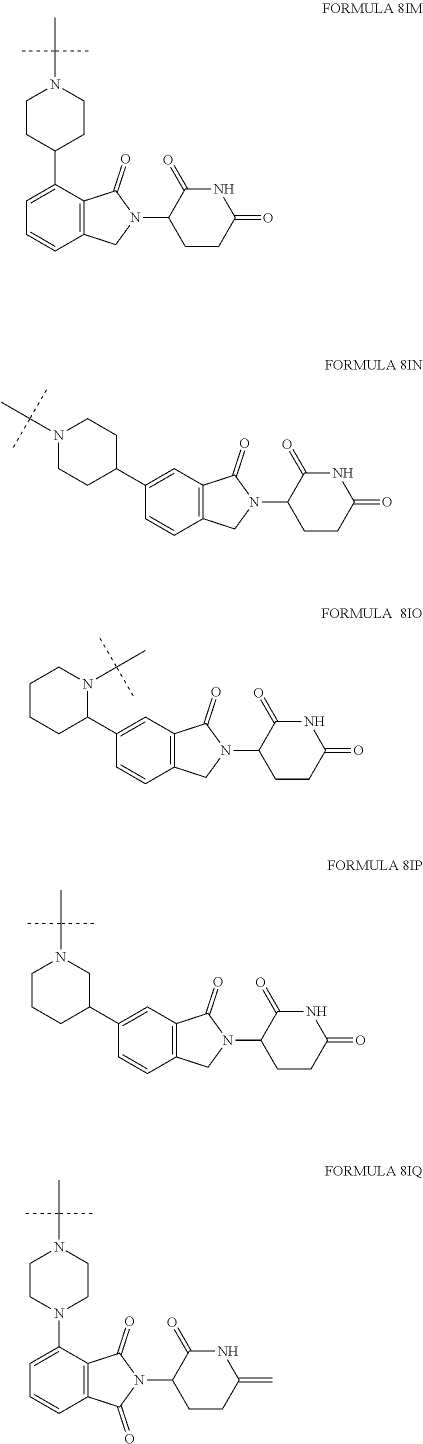

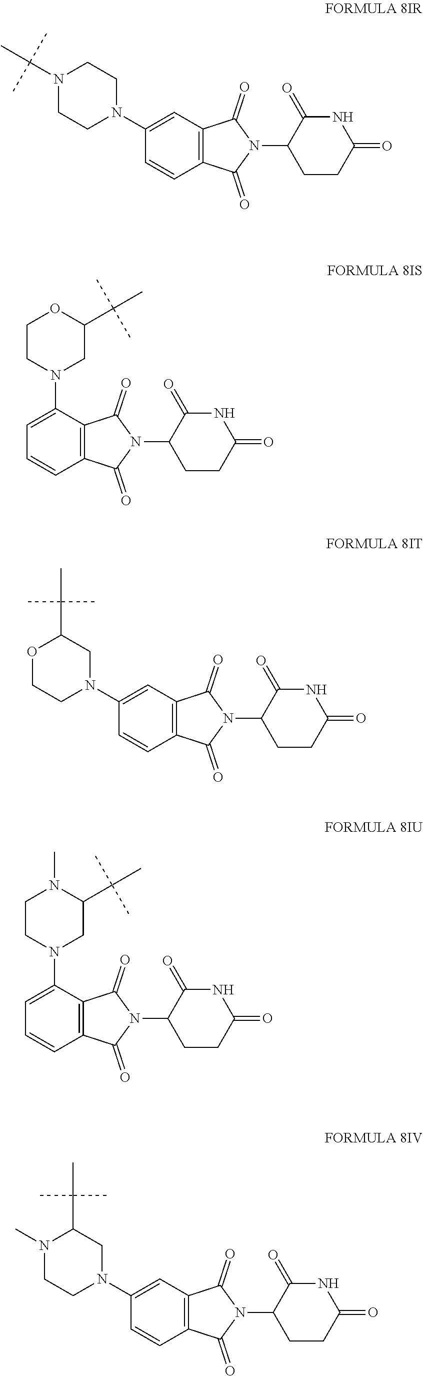

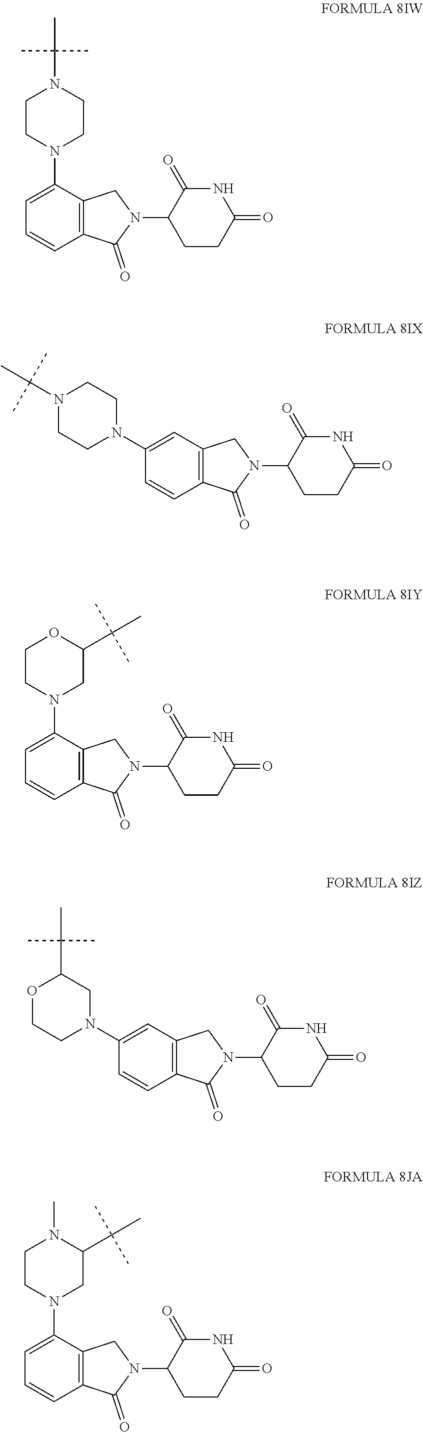

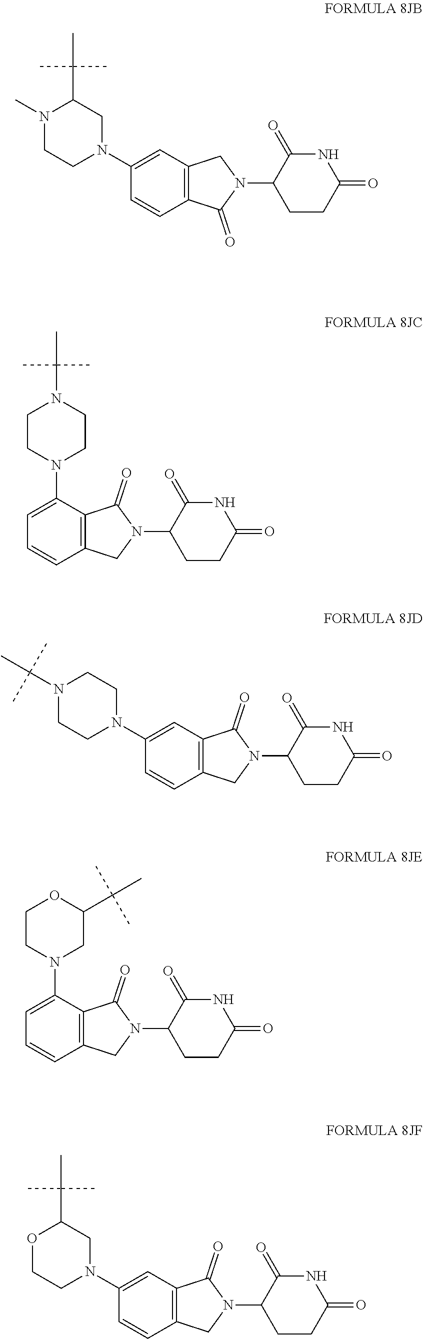

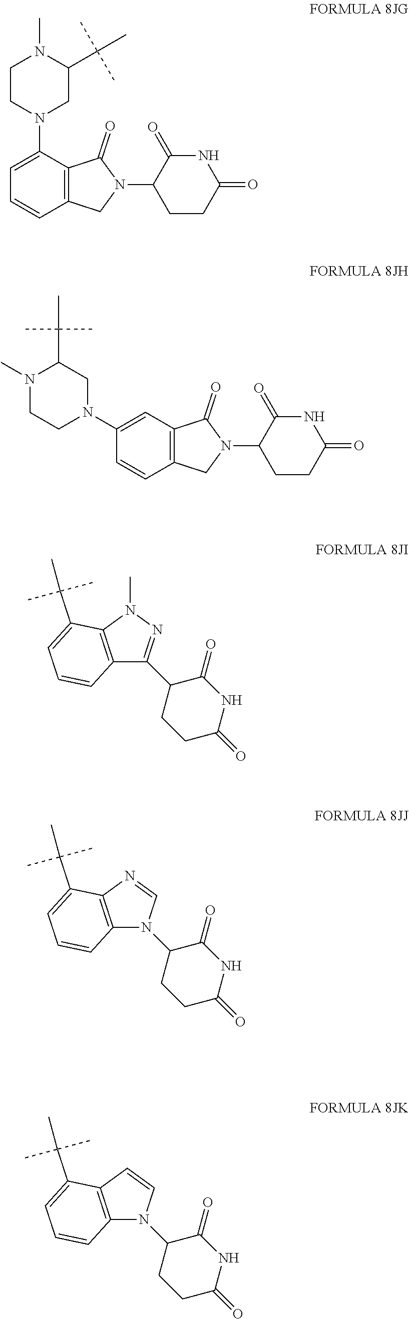

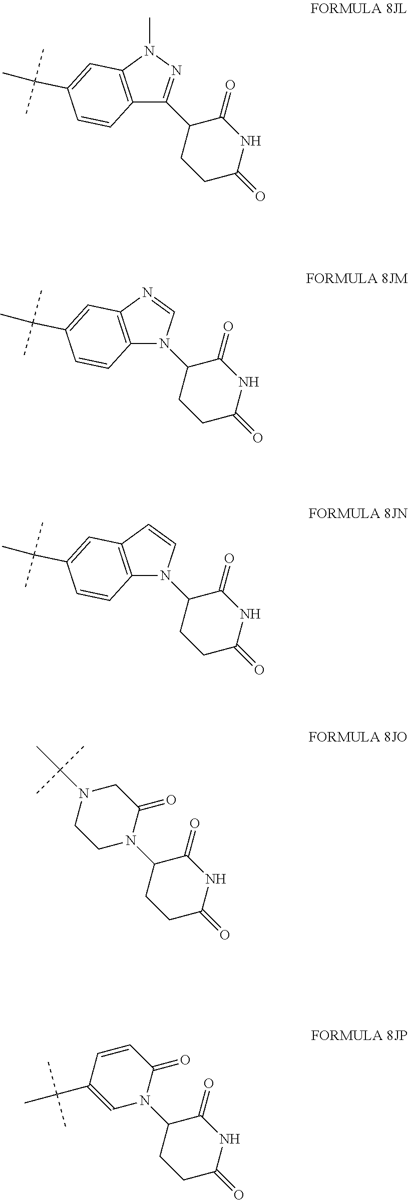

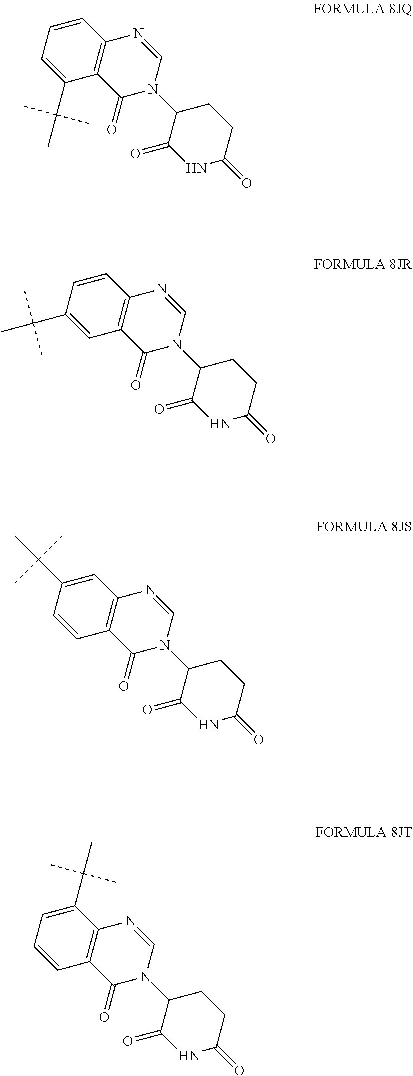

In another embodiment, the degradation tag is selected from the group consisting of: FORMULA 8A, 8B, 8C, 8D, 8E, 8F, 8G, 8H, 8I, 8J, 8K, 8L, 8M, 8O, 8P, 8Q, 8R, 8AQ, 8AR, 8AS, 8AT, 8AU, 8AV, 8AW, 8AX, 8AY, 8AZ, 8BA, 8BB, 8BC, 8BD, 8BE, 8BF, 8BG, 8BH, 8BI, 8BJ, 8BK, 8BL, 8BM, and 8BN, 8BO, 8BP, 8BQ, 8BR, 8BS, 8CB, 8CC, 8CD, 8CE, 8CF, 8CG, 8CH, 8CI, 8CJ, 8CK, 8CL, 8CM, 8CN, 8CO, 8CP, 8CQ, 8CR, 8CS, 8CT, 8CU, 8CV, 8CW, 8CX, 8CY, 8CZ, 8DA, 8DB, 8DC, 8DD, 8DE, 8DF, 8DG, 8DH, 8DI, 8DJ, 8DK, 8DL, 8DM, 8DN, 8DO, 8DP, 8DQ, 8DR, 8DS, 8DT, 8DU, 8DV, 8DW, 8DX, 8DY, 8DZ, 8EA, 8EB, 8EC, 8ED, 8EE, 8EF, 8EG, 8EH, 8EI, 8EJ, 8EK, 8EL, 8EM, 8EN, 8EO, 8EP, 8EO, 8GU, 8GV, 8GW, 8GX, 8GY, 8GZ, 8HA, 8HB, 8HC, 8HD, 8HE, 8HF, 8HG, 8HH, 8HI, 8HJ, 8HK, 8HL, 8HM, 8HN, 8HO, 8HP, 8HQ, 8HR, 8HS, 8HT, 8HU, 8HV, 8HW, 8HX, 8HY, 8HZ, 8IA, 8IB, 8IC, 8ID, 81E, 8IF, 8IG, 8IH, 8II, 8IJ, 8IK, 8IL, 8IM, 8IN, 8IO, 8IP, 8IQ, 8IR, 8IS, 8IT, 8IU, 8IV, 8IW, 8IX, 8IY, 8IZ, 8JA, 8JB, 8JC, 8JD, 8JE, 8JF, 8JG, 8JH, 8JI, 8JJ, 8JK, 8JL, 8JM, 8JN, 8JO, 8JP, 8JQ, 8JR, 8JS, and 8JT.

In some embodiments, the linker moiety is of FORMULA 9:

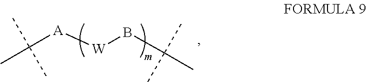

wherein

A, W and B, at each occurrence, are independently selected from null, or bivalent moiety selected from R′-R″, R′COR″, R′CO₂R″, R′C(O)N(R¹)R″, R′C(S)N(R¹)R″, R′OR″, R′SR″, R′SOR′, R′SO₂R″, R′SO₂N(R¹)R″, R′N(R¹)R″, R″N(R¹)COR″, R′N(R¹)CON(R²)R″, R′N(R¹)C(S)R″, optionally substituted C₁-C₈ alkylene, optionally substituted C₂-C₈ alkenylene, optionally substituted C₂-C₈ alkynylene, optionally substituted C₁-C₈alkoxyC₁-C₈alkylene, optionally substituted C₁-C₈ haloalkylene, optionally substituted C₁-C₈ hydroxyalkylene, optionally substituted C₃-C₁₃ fused cycloalkyl, optionally substituted C₃-C₁₃ fused heterocyclyl, optionally substituted C₃-C₁₃ bridged cycloalkyl, optionally substituted C₃-C₁₃ bridged heterocyclyl, optionally substituted C₃-C₁₃ spiro cycloalkyl, optionally substituted C₃-C₁₃ spiro heterocyclyl, optionally substituted 3-10 membered carbocyclyl, optionally substituted 4-10 membered heterocyclyl, optionally substituted aryl, and optionally substituted heteroaryl, wherein

R′ and R″ are independently selected from null, optionally substituted (C₁-C₈ alkylene)-R^{r} (preferably, CH₂-R^{r}), optionally substituted R^{r}—(C₁-C₈ alkylene), optionally substituted (C₁-C₈ alkylene)-R^{r}—(C₁-C₈ alkyl), or a moiety comprising of optionally substituted C₁-C₈ alkyl, optionally substituted C₂-C₈ alkenyl, optionally substituted C₂-C₈ alkynyl, optionally substituted C₁-C₈ hydroxyalkyl, optionally substituted C₁-C₈alkoxyC₁-C₈alkyl, optionally substituted C₁-C₈alkylaminoC₁-C₈alkyl, optionally substituted C₁-C₈ haloalkyl, optionally substituted C₁-C₈ alkylene, optionally substituted C₂-C₈ alkenylene, optionally substituted C₂-C₈ alkynylene, optionally substituted C₁-C₈ hydroxyalkylene, optionally substituted C₁-C₈alkoxyC₁-C₈alkylene, optionally substituted C₁-C₈alkylaminoC₁-C₈alkylene, optionally substituted C₁-C₈ haloalkylene, optionally substituted 3-10 membered carbocyclyl, optionally substituted 4-10 membered heterocyclyl, optionally substituted C₃-C₁₃ fused cycloalkyl, optionally substituted C₃-C₁₃ fused heterocyclyl, optionally substituted C₃-C₁₃ bridged cycloalkyl, optionally substituted C₃-C₁₃ bridged heterocyclyl, optionally substituted C₃-C₁₃ spiro cycloalkyl, optionally substituted C₃-C₁₃ spiro heterocyclyl, optionally substituted aryl, and optionally substituted heteroaryl;

R^{r} is selected from optionally substituted 3-10 membered carbocyclyl, optionally substituted 4-10 membered heterocyclyl, optionally substituted C₃-C₁₃ fused cycloalkyl, optionally substituted C₃-C₁₃ fused heterocyclyl, optionally substituted C₃-C₁₃ bridged cycloalkyl, optionally substituted C₃-C₁₃ bridged heterocyclyl, optionally substituted C₃-C₁₃ spiro cycloalkyl, optionally substituted C₃-C₁₃ spiro heterocyclyl, optionally substituted aryl, and optionally substituted heteroaryl;

R¹ and R² are independently selected from hydrogen, optionally substituted C₁-C₈ alkyl, optionally substituted C₂-C₈ alkenyl, optionally substituted C₂-C₈ alkynyl, optionally substituted C₁-C₈ alkoxyalkyl, optionally substituted C₁-C₈ haloalkyl, optionally substituted C₁-C₈ hydroxyalkyl, optionally substituted C₁-C₈alkylaminoC₁-C₈alkyl, optionally substituted 3-10 membered carbocyclyl, optionally substituted 4-10 membered heterocyclyl, optionally substituted aryl, and optionally substituted heteroaryl;

R′ and R″, R¹ and R², R′ and R¹, R′ and R², R″ and R¹, R″ and R² together with the atom to which they are connected form a 3-20 membered cycloalkyl or 4-20 membered heterocyclyl ring; and

m is 0 to 15.

In another embodiment, W and m are defined as above; and A and B, at each occurrence, are independently selected from null, CO, NH, NH CO, CO—NH, —(CH₂)₀₋₈—, —(CH₂)₀₋₃—CO—(CH₂)₀₋₈—, (CH₂)₀₋₈—NH—CO, (CH₂)₀₋₈—CO—NH, NH—CO—(CH₂)₀₋₈, CO—NH—(CH₂)₀₋₈, (CH₂)₁₋₃—NH—(CH₂)₁₋₃—CO—NH, (CH₂)₁₋₃—NH—(CH₂)₁₋₃—NH—CO, —CO—NH, CO—NH—(CH₂)₁₋₃—NH—(CH₂)₁₋₃, (CH₂)₁₋₃—NH—(CH₂)₁₋₃, —(CH₂)₀₋₃—R^{r}—(CH₂)₀₋₃, —(CH₂)₀₋₃—(CO)—(CH₂)₀₋₃−R^{r}—(CH₂)₀₋₃—, —(CH₂)₀₋₃—(CO—NH)—(CH₂)₀₋₃—R^{r}—(CH₂)₀₋₃—, —(CH₂)₀₋₃—(NH—CO)—(CH₂)₀₋₃—R^{r}—(CH₂)₀₋₃—, and —(CH₂)₀₋₃—(NH)—(CH₂)₀₋₃—R^{r}—(CH₂)₀₋₃—.

In another embodiment, R^{r} is selected from FORMULA C1a, C2a, C3a, C4a, and C5a as defined hereinafter.

In another embodiment, R^{r} is selected from FORMULA C1, C2, C3, C4, and C5 as defined hereinafter.

In another embodiment, R^{r} is selected from Group R, and Group R consists of

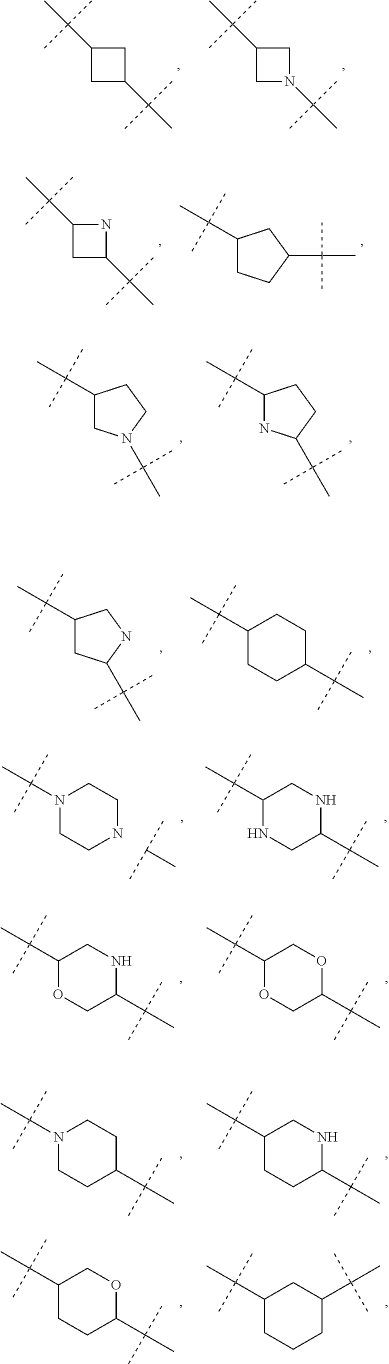

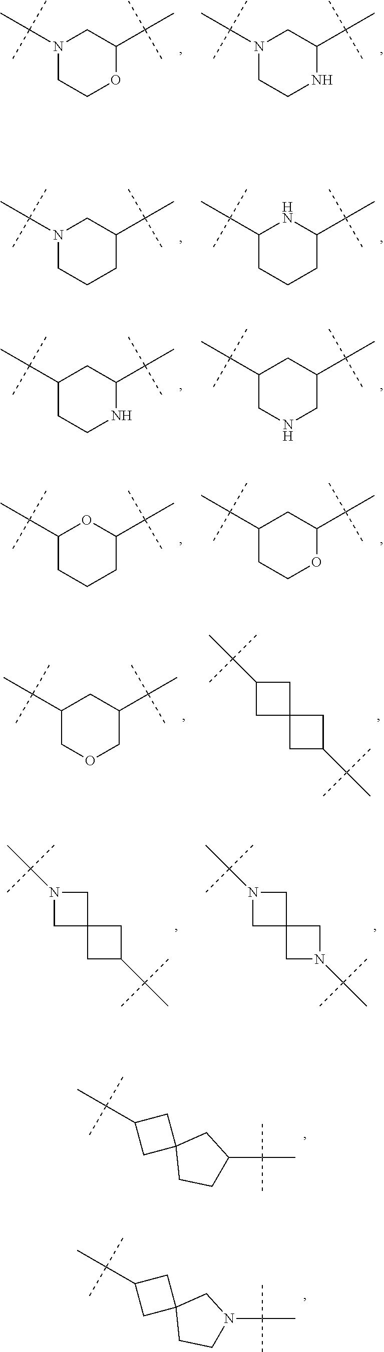

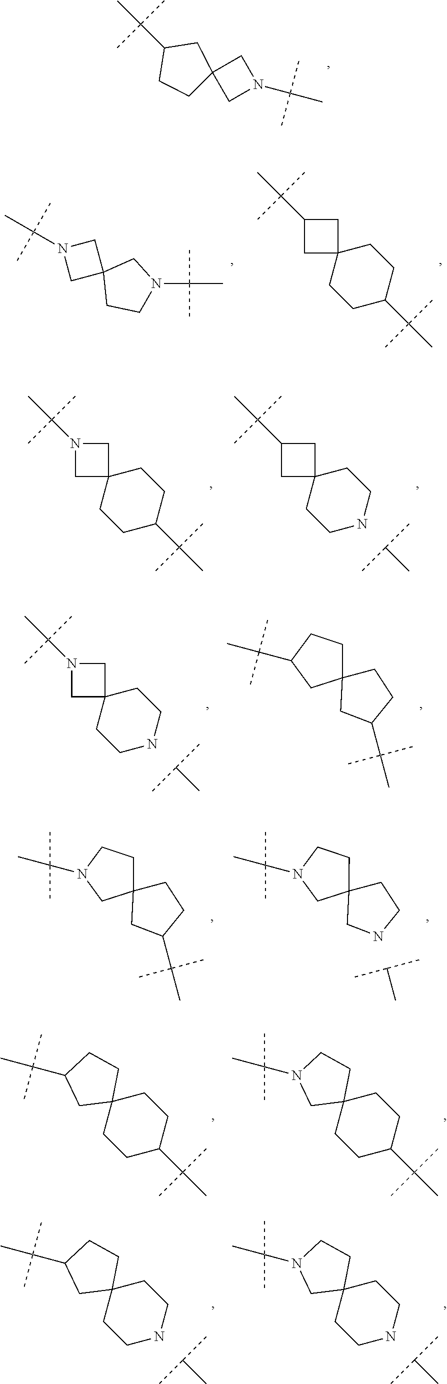

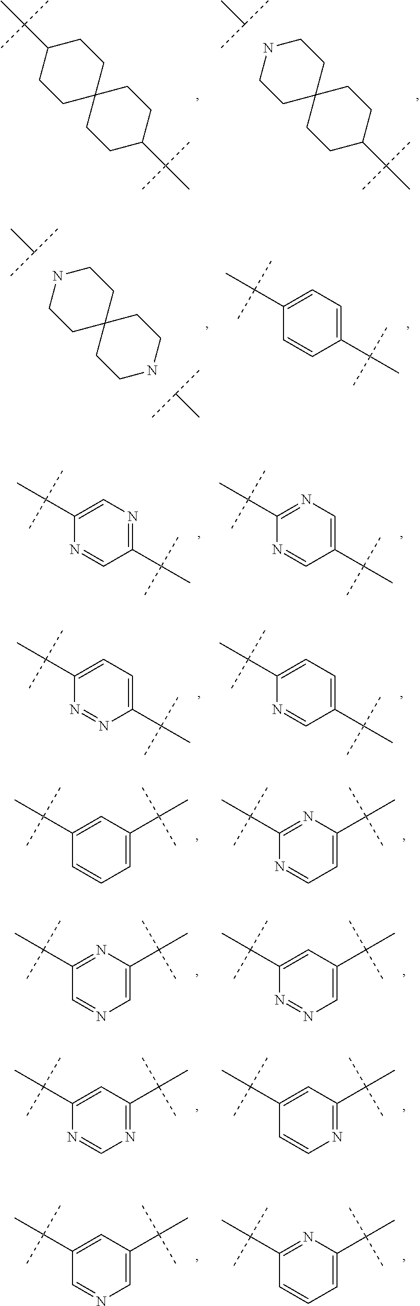

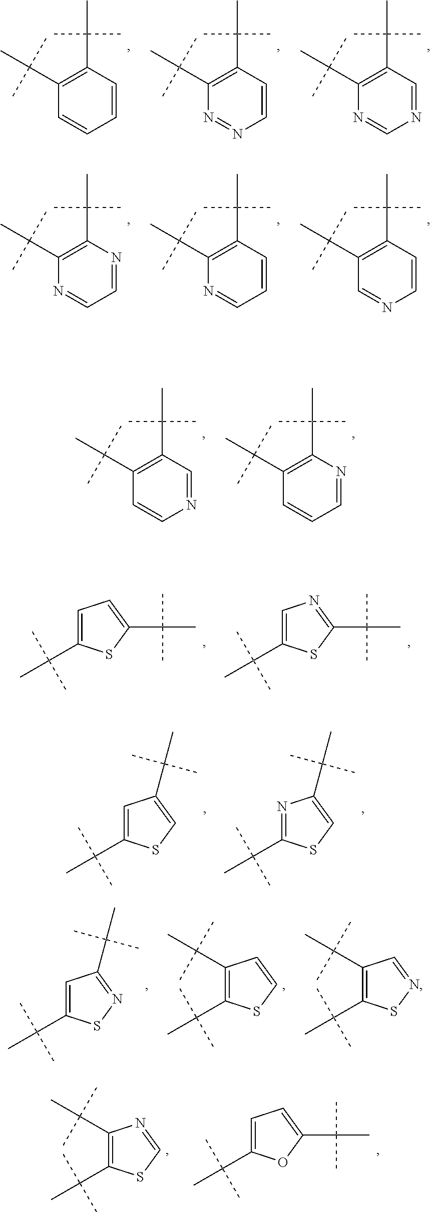

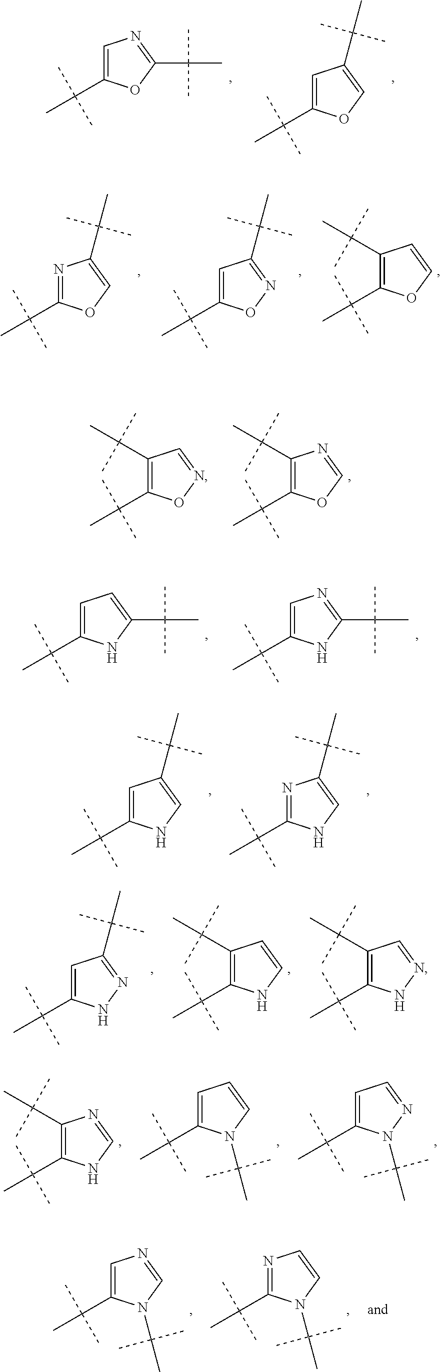

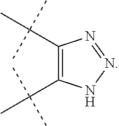

In one embodiment, the linker moiety is of FORMULA 9A:

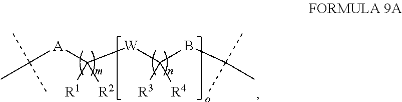

wherein

R¹, R², R³ and R⁴, at each occurrence, are independently selected from hydrogen, halogen, hydroxyl, amino, cyano, nitro, optionally substituted C₁-C₈ alkyl, optionally substituted C₂-C₈ alkenyl, optionally substituted C₂-C₈ alkynyl, optionally substituted C₁-C₈ alkoxy, optionally substituted C₁-C₈ alkoxyalkyl, optionally substituted C₁-C₈ haloalkyl, optionally substituted C₁-C₈ hydroxyalkyl, optionally substituted C₁-C₈ alkylamino, and optionally substituted C₁-C₈ alkylaminoC₁-C₈ alkyl, optionally substituted 3-10 membered carbocyclyl, optionally substituted 3-8 membered cycloalkoxy, optionally substituted 3-10 membered carbocyclylamino, optionally substituted 4-8 membered heterocyclyl, optionally substituted aryl, and optionally substituted heteroaryl, or

R¹ and R², R³ and R⁴ together with the atom to which they are connected form a 3-20 membered cycloalkyl or 4-20 membered heterocyclyl ring;

A, W and B, at each occurrence, are independently selected from null, or bivalent moiety selected from R′-R″, R′COR″—, R′CO₂R″, R′C(O)N(R⁵)R″, R′C(S)N(R⁵)R″, R′OR″, R′SR″, R′SOR″, R′SO₂R″, R′SO₂N(R⁵)R″, R′N(R⁵)R″, R′N(R⁵)COR″, R′N(R⁵)CON(R⁶)R″, R′N(R⁵)C(S)R″, optionally substituted C₁-C₈ alkylene, optionally substituted C₂-C₈ alkenylene, optionally substituted C₂-C₈ alkynylene, optionally substituted C₁-C₈alkoxyC₁-C₈alkylene, optionally substituted C₁-C₈ haloalkylene, optionally substituted C₁-C₈ hydroxyalkylene, optionally substituted C₃-C₁₃ fused cycloalkyl, optionally substituted C₃-C₁₃ fused heterocyclyl, optionally substituted C₃-C₁₃ bridged cycloalkyl, optionally substituted C₃-C₁₃ bridged heterocyclyl, optionally substituted C₃-C₁₃ spiro cycloalkyl, optionally substituted C₃-C₁₃ spiro heterocyclyl, optionally substituted 3-10 membered carbocyclyl, optionally substituted 4-10 membered heterocyclyl, optionally substituted aryl, and optionally substituted heteroaryl, wherein

R′ and R″ are independently selected from null, optionally substituted (C₁-C₈ alkylene)-R^{r} (preferably, CH₂-R^{r}), optionally substituted R^{r}—(C₁-C₈ alkylene), optionally substituted (C₁-C₈ alkylene)-R^{r}—(C₁-C₈ alkylene), or a moiety comprising of optionally substituted C₁-C₈ alkyl, optionally substituted C₂-C₈ alkenyl, optionally substituted C₂-C₈ alkynyl, optionally substituted C₁-C₈ hydroxyalkyl, optionally substituted C₁-C₈alkoxyC₁-C₈alkyl, optionally substituted C₁-C₈alkylaminoC₁-C₈alkyl, optionally substituted C₁-C₈ haloalkyl, optionally substituted C₁-C₈ alkylene, optionally substituted C₂-C₈ alkenylene, optionally substituted C₂-C₈ alkynylene, optionally substituted C₁-C₈ hydroxyalkylene, optionally substituted C₁-C₈alkoxyC₁-C₈alkylene, optionally substituted C₁-C₈alkylaminoC₁-C₈alkylene, optionally substituted C₁-C₈ haloalkylene, optionally substituted 3-10 membered carbocyclyl, optionally substituted 4-10 membered heterocyclyl, optionally substituted C₃-C₁₃ fused cycloalkyl, optionally substituted C₃-C₁₃ fused heterocyclyl, optionally substituted C₃-C₁₃ bridged cycloalkyl, optionally substituted C₃-C₁₃ bridged heterocyclyl, optionally substituted C₃-C₁₃ spiro cycloalkyl, optionally substituted C₃-C₁₃ spiro heterocyclyl, optionally substituted aryl, and optionally substituted heteroaryl;

R^{r} is selected from optionally substituted 3-10 membered carbocyclyl, optionally substituted 4-10 membered heterocyclyl, optionally substituted C₃-C₁₃ fused cycloalkyl, optionally substituted C₃-C₁₃ fused heterocyclyl, optionally substituted C₃-C₁₃ bridged cycloalkyl, optionally substituted C₃-C₁₃ bridged heterocyclyl, optionally substituted C₃-C₁₃ spiro cycloalkyl, optionally substituted C₃-C₁₃ spiro heterocyclyl, optionally substituted aryl, and optionally substituted heteroaryl;

R⁵ and R⁶ are independently selected from hydrogen, optionally substituted C₁-C₈ alkyl, optionally substituted C₂-C₈ alkenyl, optionally substituted C₂-C₈ alkynyl, optionally substituted C₁-C₈ alkoxyalkyl, optionally substituted C₁-C₈ haloalkyl, optionally substituted C₁-C₈ hydroxyalkyl, optionally substituted C₁-C₈alkylaminoC₁-C₈alkyl, optionally substituted 3-10 membered carbocyclyl, optionally substituted 4-10 membered heterocyclyl, optionally substituted aryl, and optionally substituted heteroaryl;

R′ and R″, R⁵ and R⁶, R′ and R⁵, R′ and R⁶, R″ and R⁵, R″ and R⁶ together with the atom to which they are connected form a 3-20 membered cycloalkyl or 4-20 membered heterocyclyl ring;

m is 0 to 15;

n, at each occurrence, is 0 to 15; and

o is 0 to 15.

In another embodiment, A, W and B, at each occurrence, are independently selected from null, CO, NH, NH—CO, CO—NH, —(CH₂)₀₋₈—, —(CH₂)₀₋₃—CO—(CH₂)₀₋₈—, (CH₂)₀₋₈—NH—CO, (CH₂)₀₋₈—CO—NH, NH—CO—(CH₂)₀₋₈, CO—NH—(CH₂)₀₋₈, (CH₂)₁₋₃—NH—(CH₂)₁₋₃—CO—NH, (CH₂)₁₋₃—NH—(CH₂)₁₋₃—NH—CO, —CO—NH, CO—NH—(CH₂)₁₋₃—NH—(CH₂)₁₋₃, (CH₂)₁₋₃—NH—(CH₂)₁₋₃, —(CH₂)₀₋₃—R^{r}—(CH₂)₀₋₃, —(CH₂)₀₋₃—(CO)—(CH₂)₀₋₃—R^{r}—(CH₂)₀₋₃—, —(CH₂)₀₋₃—(CO—NH)—(CH₂)₀₋₃—R^{r}—(CH₂)₀₋₃—, —(CH₂)₀₋₃—(NH—CO)—(CH₂)₀₋₃—R^{r}—(CH₂)₀₋₃—, and —(CH₂)₀₋₃—(NH)—(CH₂)₀₋₃—R^{r}—(CH₂)₀₋₃—.

In another embodiment, R^{r} is selected from FORMULA C1a, C2a, C3a, C4a, and C5a as defined hereinafter.

In another embodiment, R^{r} is selected from FORMULA C1, C2, C3, C4, and C5 as defined hereinafter.

In another embodiment, R^{r} is selected from Group R, and Group R is defined as in FORMULA 9.

In one embodiment, the CBP/P300 ligand of the bivalent compound is attached to A in FORMULA 9A.

In another embodiment, A (when A is attached to the CBP/P300 ligand) is selected from null, CO, NH, NH—CO, CO—NH, —(CH₂)₀₋₈—, —(CH₂)₀₋₃-CO—(CH₂)₀₋₈—, (CH₂)₀₋₈—NH—CO, (CH₂)₀₋₈—CO—NH, NH—CO—(CH₂)₀₋₈, CO—NH—(CH₂)₀₋₈, (CH₂)₀₋₈—NH—(CH₂)₀₋₈-CO—NH, (CH₂)₁₋₃—NH—(CH₂)₁₋₃—NH—CO, CO—NH—(CH₂)₁₋₃—NH—(CH₂)₁₋₃, (CH₂)₁₋₃—NH—(CH₂)₁₋₃, —(CH₂)₀₋₃—R^{r}—(CH₂)₀₋₃, —(CH₂)₀₋₃—(CO)—(CH₂)₀₋₃—R^{r}—(CH₂)₀₋₃—, —(CH₂)₀₋₃—(CO—NH)—(CH₂)₀₋₃—R^{r}—(CH₂)₀₋₃—, —(CH₂)₀₋₃—(NH—CO)—(CH₂)₀₋₃—R^{r}—(CH₂)₀₋₃—, —(CH₂)₀₋₃—(NH)—(CH₂)₀₋₃—R^{r}—(CH₂)₀₋₃—, wherein

R^{r} is selected from Group R, and Group R is defined as in FORMULA 9; and

W and B is null.

In one embodiment, the linker moiety is of FORMULA 9A:

wherein

R¹, R², R³ and R⁴, at each occurrence, are independently selected from hydrogen, optionally substituted C₁-C₈ alkyl (preferably, C₁-C₄ alkyl), or

R¹ and R², R³ and R⁴ together with the atom to which they are connected form a 3-20 membered cycloalkyl (preferably, 3-5 membered cycloalkyl) or 4-20 membered heterocyclyl ring;

A is defined as before; and W and B are null;

m is 0 to 15 (preferably, m is 0, 1, or 2);

n, at each occurrence, is 1 to 15 (preferably, n is 1); and

o is 1 to 15 (preferably, o is 1, 2, 3, 4, 5, 6, 7, 8, 9, 10, 11, 12 or 13).

In another embodiment, A is independently selected from null, or bivalent moiety selected from R′-R″, R′COR, R′CO₂R″, R′C(O)N(R⁵)R′, R′C(S)N(R⁵)R′, R′OR″, R′SR″, R′SOR″, R′SO₂R′, R′SO₂N(R⁵)R′, R′N(R⁵)R″, R′N(R⁵)COR″, R′N(R⁵)CON(R⁶)R″, R′N(R⁵)C(S)R″; R′ and R″ are defined as above.

In another embodiment, R′ and R″ are independently selected from null, optionally substituted (C₁-C₈ alkyl)-R^{r} (preferably, CH₂—R^{r}), or optionally substituted C₁-C₈ alkyl (preferably, optionally substituted C₁-C₂ alkyl).

In another embodiment, the linker moiety is of FORMULA 9B:

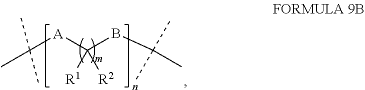

wherein

R¹ and R², at each occurrence, are independently selected from hydrogen, halogen, hydroxyl, amino, cyano, nitro, and optionally substituted C₁-C₈ alkyl, optionally substituted C₁-C₈ alkoxy, optionally substituted C₁-C₈ alkoxy C₁-C₈ alkyl, optionally substituted C₁-C₈ haloalkyl, optionally substituted C₁-C₈ hydroxyalkyl, optionally substituted C₁-C₈ alkylamino, C₁-C₈alkylaminoC₁-C₈alkyl, optionally substituted 3-10 membered carbocyclyl, optionally substituted 3-8 membered cycloalkoxy, optionally substituted 3-10 membered carbocyclylamino, optionally substituted 4-10 membered heterocyclyl, optionally substituted aryl, and optionally substituted heteroaryl, or

R¹ and R² together with the atom to which they are connected form a 3-20 membered cycloalkyl or 4-20 membered heterocyclyl ring;

A and B, at each occurrence, are independently selected from null, or bivalent moiety selected from R′-R″, R′COR″, R′CO₂R″, R′C(O)N(R³)R″, R′C(S)N(R³)R″, R′OR″, R′SR″, R′SOR″, R′SO₂R″, R′SO₂N(R³)R″, R′N(R³)R″, R′N(R³)COR″, R′N(R³)CON(R⁴)R″, R′N(R³)C(S)R″, optionally substituted C₁-C₈ alkylene, optionally substituted C₂-C₈ alkenylene, optionally substituted C₂-C₈ alkynylene, optionally substituted C₁-C₈alkoxyC₁-C₈alkylene, optionally substituted C₁-C₈ haloalkylene, optionally substituted C₁-C₈ hydroxyalkylene, optionally substituted C₃-C₁₃ fused cycloalkyl, optionally substituted C₃-C₁₃ fused heterocyclyl, optionally substituted C₃-C₁₃ bridged cycloalkyl, optionally substituted C₃-C₁₃ bridged heterocyclyl, optionally substituted C₃-C₁₃ spiro cycloalkyl, optionally substituted C₃-C₁₃ spiro heterocyclyl, optionally substituted 3-10 membered carbocyclyl, optionally substituted 4-10 membered heterocyclyl, optionally substituted aryl, and optionally substituted heteroaryl, wherein

R′ and R″ are independently selected from null, optionally substituted (C₁-C₈ alkylene)-R^{r} (preferably, CH₂-R^{r}), optionally substituted R^{r}—(C₁-C₈ alkylene), optionally substituted (C₁-C₈ alkylene)-R^{r}—(C₁-C₈ alkylene), or a moiety comprising of optionally substituted C₁-C₈ alkyl, optionally substituted C₂-C₈ alkenyl, optionally substituted C₂-C₈ alkynyl, optionally substituted C₁-C₈ hydroxyalkyl, optionally substituted C₁-C₈alkoxyC₁-C₈alkyl, optionally substituted C₁-C₈alkylaminoC₁-C₈alkyl, optionally substituted C₁-C₈ haloalkyl, optionally substituted C₁-C₈ alkylene, optionally substituted C₂-C₈ alkenylene, optionally substituted C₂-C₈ alkynylene, optionally substituted C₁-C₈ hydroxyalkylene, optionally substituted C₁-C₈alkoxyC₁-C₈alkylene, optionally substituted C₁-C₈alkylaminoC₁-C₈alkylene, optionally substituted C₁-C₈ haloalkylene, optionally substituted 3-10 membered carbocyclyl, optionally substituted 4-10 membered heterocyclyl, optionally substituted C₃-C₁₃ fused cycloalkyl, optionally substituted C₃-C₁₃ fused heterocyclyl, optionally substituted C₃-C₁₃ bridged cycloalkyl, optionally substituted C₃-C₁₃ bridged heterocyclyl, optionally substituted C₃-C₁₃ spiro cycloalkyl, optionally substituted C₃-C₁₃ spiro heterocyclyl, optionally substituted aryl, and optionally substituted heteroaryl;

R^{r} is selected from optionally substituted 3-10 membered carbocyclyl, optionally substituted 4-10 membered heterocyclyl, optionally substituted C₃-C₁₃ fused cycloalkyl, optionally substituted C₃-C₁₃ fused heterocyclyl, optionally substituted C₃-C₁₃ bridged cycloalkyl, optionally substituted C₃-C₁₃ bridged heterocyclyl, optionally substituted C₃-C₁₃ spiro cycloalkyl, optionally substituted C₃-C₁₃ spiro heterocyclyl, optionally substituted aryl, and optionally substituted heteroaryl;

R³ and R⁴ are independently selected from hydrogen, optionally substituted C₁-C₈ alkyl, optionally substituted C₂-C₈ alkenyl, optionally substituted C₂-C₈ alkynyl, optionally substituted C₁-C₈ alkoxyalkyl, optionally substituted C₁-C₈ haloalkyl, optionally substituted C₁-C₈ hydroxyalkyl, optionally substituted C₁-C₈alkylaminoC₁-C₈alkyl, optionally substituted 3-10 membered carbocyclyl, optionally substituted 4-10 membered heterocyclyl, optionally substituted aryl, and optionally substituted heteroaryl;

R′ and R″, R³ and R⁴, R′ and R³, R′ and R⁴, R″ and R³, R″ and R⁴ together with the atom to which they are connected form a 3-20 membered cycloalkyl or 4-20 membered heterocyclyl ring;

each m is 0 to 15; and

n is 0 to 15.

In another embodiment, A and B, at each occurrence, are independently selected from null, CO, NH, NH—CO, CO—NH, —(CH₂)₀₋₈—, —(CH₂)₀₋₃-CO—(CH₂)₀₋₈—, (CH₂)₀₋₈—NH—CO, (CH₂)₀₋₈—CO—NH, NH—CO—(CH₂)₀₋₈, CO—NH—(CH₂)₀₋₈, (CH₂)₁₋₃—NH—(CH₂)₁₋₃—CO—NH, (CH₂)₁₋₃—NH—(CH₂)₁₋₃—NH—CO, CO—NH—(CH₂)₁₋₃—NH—(CH₂)₁₋₃, (CH₂)₁₋₃—NH—(CH₂)₁₋₃, —(CH₂)₀₋₃—R^{r}—(CH₂)₀₋₃, —(CH₂)₀₋₃—(CO)—(CH₂)₀₋₃—R^{r}—(CH₂)₀₋₃—, —(CH₂)₀₋₃—(CO—NH)—(CH₂)₀₋₃—R^{r}—(CH₂)₀₋₃—, —(CH₂)₀₋₃—(NH—CO)—(CH₂)₀₋₃—R^{r}—(CH₂)₀₋₃—, —(CH₂)₀₋₃—(NH)—(CH₂)₀₋₃—R^{r}—(CH₂)₀₋₃—.

In another embodiment, R^{r} is selected from FORMULA C1a, C2a, C3a, C4a, and C5a as defined hereinafter.

In another embodiment, R^{r} is selected from FORMULA C1, C2, C3, C4, and C5 as defined hereinafter.

In another embodiment, Rᵣ is selected from Group R, and Group R is defined as in FORMULA 9.

In another embodiment, the linker moiety is of FORMULA 9C:

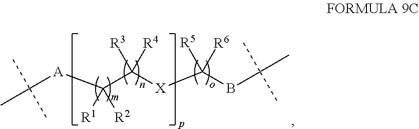

wherein

X is selected from O, NH, and NR′;

R¹, R², R³, R⁴, R⁵, and R⁶, at each occurrence, are independently selected from hydrogen, halogen, hydroxyl, amino, cyano, nitro, optionally substituted C₁-C₈ alkyl, optionally substituted C₂-C₈ alkenyl, optionally substituted C₂-C₈ alkynyl, optionally substituted C₁-C₈ alkoxy, optionally substituted C₁-C₈ alkoxy C₁-C₈ alkyl, optionally substituted C₁-C₈ haloalkyl, optionally substituted C₁-C₈ hydroxyalkyl, optionally substituted C₁-C₈ alkylamino, optionally substituted C₁-C₈ alkylaminoC₁-C₈ alkyl, optionally substituted 3-10 membered carbocyclyl, optionally substituted 3-8 membered cycloalkoxy, optionally substituted 4-10 membered heterocyclyl, optionally substituted aryl, and optionally substituted heteroaryl;

A and B are independently selected from null, or bivalent moiety selected from R′-R″, R′COR″, R′CO₂R″, R′C(O)N(R⁸)R″, R′C(S)N(R⁸)R″, R′OR″, R′SR″, R′SOR″, R′SO₂R″, R′SO₂N(R⁸)R″, R′N(R⁸)R″, R′N(R⁸)COR″, R′N(R⁸)CON(R⁹)R″, R′N(R⁸)C(S)R″, optionally substituted C₁-C₈ alkylene, optionally substituted C₂-C₈ alkenylene, optionally substituted C₂-C₈ alkynylene, optionally substituted C₁-C₈alkoxyC₁-C₈alkylene, optionally substituted C₁-C₈ haloalkylene, optionally substituted C₁-C₈ hydroxyalkylene, optionally substituted C₃-C₁₃ fused cycloalkyl, optionally substituted C₃-C₁₃ fused heterocyclyl, optionally substituted C₃-C₁₃ bridged cycloalkyl, optionally substituted C₃-C₁₃ bridged heterocyclyl, optionally substituted C₃-C₁₃ spiro cycloalkyl, optionally substituted C₃-C₁₃ spiro heterocyclyl, optionally substituted 3-10 membered carbocyclyl, optionally substituted 4-10 membered heterocyclyl, optionally substituted aryl, and optionally substituted heteroaryl, wherein

R′ and R″ are independently selected from null, optionally substituted (C₁-C₈ alkylene)-R^{r} (preferably, CH₂-R^{r}), optionally substituted R^{r}—(C₁-C₈ alkylene), optionally substituted (C₁-C₈ alkylene)-R^{r}—(C₁-C₈ alkylene), or a moiety comprising of optionally substituted C₁-C₈ alkyl, optionally substituted C₂-C₈ alkenyl, optionally substituted C₂-C₈ alkynyl, optionally substituted C₁-C₈ hydroxyalkyl, optionally substituted C₁-C₈alkoxyC₁-C₈alkyl, optionally substituted C₁-C₈alkylaminoC₁-C₈alkyl, optionally substituted C₁-C₈ haloalkyl, optionally substituted C₁-C₈ alkylene, optionally substituted C₂-C₈ alkenylene, optionally substituted C₂-C₈ alkynylene, optionally substituted C₁-C₈ hydroxyalkylene, optionally substituted C₁-C₈alkoxyC₁-C₈alkylene, optionally substituted C₁-C₈alkylaminoC₁-C₈alkylene, optionally substituted C₁-C₈ haloalkylene, optionally substituted 3-10 membered carbocyclyl, optionally substituted 4-10 membered heterocyclyl, optionally substituted C₃-C₁₃ fused cycloalkyl, optionally substituted C₃-C₁₃ fused heterocyclyl, optionally substituted C₃-C₁₃ bridged cycloalkyl, optionally substituted C₃-C₁₃ bridged heterocyclyl, optionally substituted C₃-C₁₃ spiro cycloalkyl, optionally substituted C₃-C₁₃ spiro heterocyclyl, optionally substituted aryl, and optionally substituted heteroaryl;

R^{r} is selected from optionally substituted 3-10 membered carbocyclyl, optionally substituted 4-10 membered heterocyclyl, optionally substituted C₃-C₁₃ fused cycloalkyl, optionally substituted C₃-C₁₃ fused heterocyclyl, optionally substituted C₃-C₁₃ bridged cycloalkyl, optionally substituted C₃-C₁₃ bridged heterocyclyl, optionally substituted C₃-C₁₃ spiro cycloalkyl, optionally substituted C₃-C₁₃ spiro heterocyclyl, optionally substituted aryl, and optionally substituted heteroaryl;

R⁷, R⁸ and R⁹ are independently selected from hydrogen, optionally substituted C₁-C₈ alkyl, optionally substituted C₂-C₈ alkenyl, optionally substituted C₂-C₈ alkynyl, optionally substituted C₁-C₈ alkoxyalkyl, optionally substituted C₁-C₈ haloalkyl, optionally substituted C₁-C₈ hydroxyalkyl, optionally substituted C₁-C₈alkylaminoC₁-C₈alkyl, optionally substituted 3-10 membered carbocyclyl, optionally substituted 4-10 membered heterocyclyl, optionally substituted aryl, and optionally substituted heteroaryl;

R′ and R″, R⁸ and R⁹, R′ and R⁸, R′ and R⁹, R″ and R⁸, R″ and R⁹ together with the atom to which they are connected form a 3-20 membered cycloalkyl or 4-20 membered heterocyclyl ring;

m, at each occurrence, is 0 to 15;

n, at each occurrence, is 0 to 15;

o is 0 to 15; and

p is 0 to 15.

In another embodiment, A and B are independently selected from null, CO, NH, NH—CO, CO—NH, —(CH₂)₀₋₈—, —(CH₂)₀₋₃—CO—(CH₂)₀₋₈—, (CH₂)₀₋₈—NH—CO, (CH₂)₀₋₈—CO—NH, NH—CO—(CH₂)₀₋₈, CO—NH—(CH₂)₀₋₈, (CH₂)₁₋₃—NH—(CH₂)₁₋₃—CO—NH, (CH₂)₁₋₃—NH—(CH₂)₁₋₃—NH—CO, —CO—NH, CO—NH—(CH₂)₁₋₃—NH—(CH₂)₁₋₃, (CH₂)₁₋₃—NH—(CH₂)₁₋₃, —(CH₂)₀₋₃—R^{r}—(CH₂)₀₋₃, —(CH₂)₀₋₃—(CO)—(CH₂)₀₋₃—R^{r}—(CH₂)₀₋₃—, —(CH₂)₀₋₃—(CO—NH)—(CH₂)₀₋₃—R^{r}—(CH₂)₀₋₃—, —(CH₂)₀₋₃—(NH—CO)—(CH₂)₀₋₃—R^{r}—(CH₂)₀₋₃—, and —(CH₂)₀₋₃—(NH)—(CH₂)₀₋₃—R^{r}—(CH₂)₀₋₃—.

In another embodiment, R^{r} is selected from FORMULA C1a, C2a, C3a, C4a, and C5a as defined hereinafter.

In another embodiment, R^{r} is selected from FORMULA C1, C2, C3, C4, and C5 as defined hereinafter.

In another embodiment, R^{r} is selected from Group R, and Group R is defined as in FORMULA 9.

In another embodiment, A and B, at each occurrence, are independently selected from null, CO, NH, NH—CO, CO—NH, CH₂—NH—CO, CH₂—CO—NH, NH—CO—CH₂, CO—NH—CH₂, CH₂—NH—CH₂—CO—NH, CH₂—NH—CH₂—NH—CO, —CO—NH, CO—NH—CH₂—NH—CH₂, CH₂—NH—CH₂,

In another embodiment, o is 0 to 5.

In another embodiment, the linker moiety comprises one or more rings selected from the group consisting of 3 to 13 membered rings, 3 to 13 membered fused rings, 3 to 13 membered bridged rings, and 3 to 13 membered spiro rings.

In another embodiment, the linker moiety comprises one or more rings selected from the group consisting of FORMULAE C1a, C2a, C3a, C4a and C5a

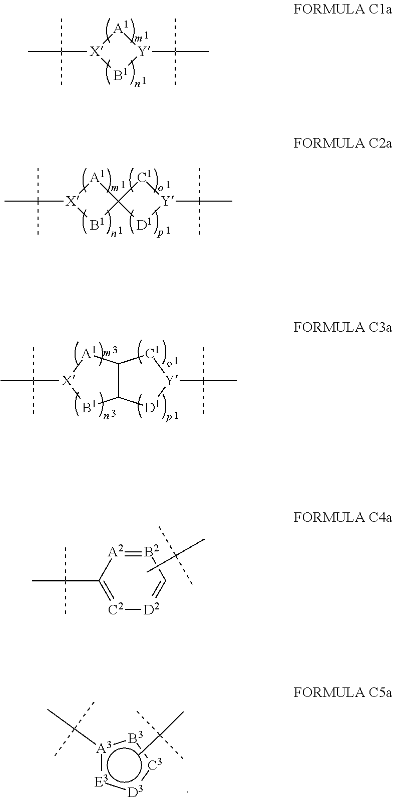

wherein

X′ and Y′ are independently selected from N, CR^{b};

A¹, B¹, C¹ and D¹, at each occurrence, are independently selected from null, O, CO, SO, SO₂, NR^{b}, CR^{b}R^{c};

A², B², C², and D², at each occurrence, are independently selected from N, CR^{b};

A³, B³, C³, D³, and E³, at each occurrence, are independently selected from N, O, S, NR^{b}, CR^{b};

R^{b} and R^{c}, at each occurrence, are independently selected from hydrogen, halogen, hydroxyl, amino, cyano, nitro, optionally substituted C₁-C₈ alkyl, optionally substituted C₂-C₈ alkenyl, optionally substituted C₂-C₈ alkynyl, optionally substituted C₁-C₈ alkoxy, optionally substituted C₁-C₈ alkoxyalkyl, optionally substituted C₁-C₈ haloalkyl, optionally substituted C₁-C₈ hydroxyalkyl, optionally substituted C₁-C₈ alkylamino, and optionally substituted C₁-C₈ alkylaminoC₁-C₈ alkyl, optionally substituted 3-10 membered carbocyclyl, optionally substituted 3-8 membered cycloalkoxy, optionally substituted 3-10 membered carbocyclylamino, optionally substituted 4-8 membered heterocyclyl, optionally substituted aryl, and optionally substituted heteroaryl; and

m¹, n¹, o¹ and p¹ are independently selected from 0, 1, 2, 3, 4 and 5.

In another embodiment, the linker moiety comprises one or more rings selected from the group consisting of FORMULAE C1, C2, C3, C4 and C5:

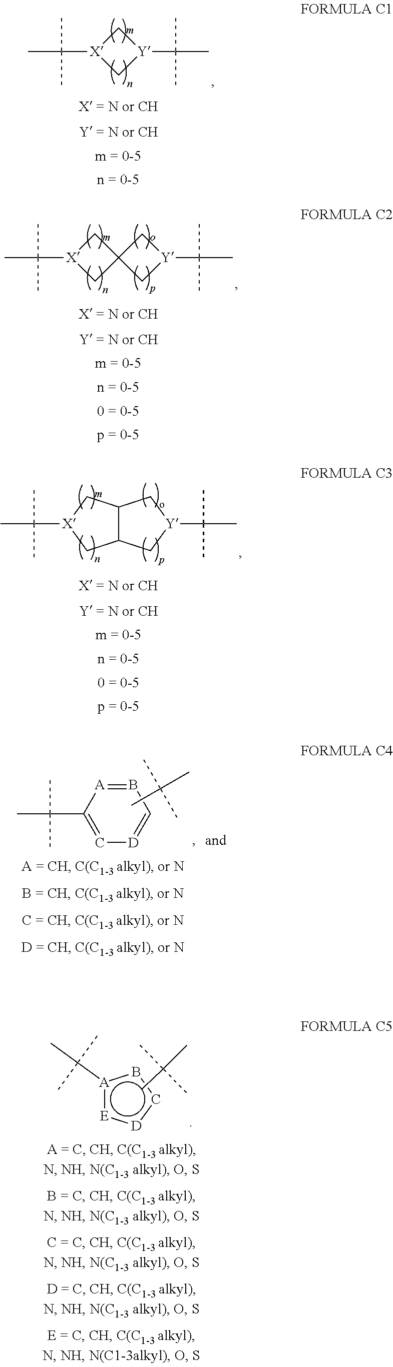

In another embodiment, the linker moiety is of FORMULA 9A.

In another embodiment, A, W and B, at each occurrence, are independently selected from null, CO, NH, NH—CO, CO—NH, —(CH₂)₀₋₈—, —(CH₂)₀₋₃—CO—(CH₂)₀₋₈—, (CH₂)₀₋₈—NH—CO, (CH₂)₀₋₈—CO—NH, NH—CO—(CH₂)₀₋₈, CO—NH—(CH₂)₀₋₈, (CH₂)₁₋₃—NH—(CH₂)₁₋₃—CO—NH, (CH₂)₁₋₃—NH—(CH₂)₁₋₃—NH—CO, CO—NH—(CH₂)₁₋₃—NH—(CH₂)₁₋₃, (CH₂)₁₋₃—NH—(CH₂)₁₋₃, —(CH₂)₀₋₃—R^{r}—(CH₂)₀₋₃, —(CH₂)₀₋₃—(CO)—(CH₂)₀₋₃—R^{r}—(CH₂)₀₋₃—, —(CH₂)₀₋₃—(CO—NH)—(CH₂)₀₋₃—R^{r}—(CH₂)₀₋₃—, —(CH₂)₀₋₃—(NH—CO)—(CH₂)₀₋₃—R^{r}—(CH₂)₀₋₃—, and —(CH₂)₀₋₃—(NH)—(CH₂)₀₋₃—R^{r}—(CH₂)₀₋₃—;

In another embodiment, R^{r} is selected from FORMULA C1a, C2a, C3a, C4a, and C5a as defined above.

In another embodiment, R^{r} is selected from FORMULA C1, C2, C3, C4, and C5 as defined above.

In another embodiment, R^{r} is selected from Group R, and Group R is defined as in FORMULA 9.

In another embodiment, in FORMULA 9A, A and B are independently defined as above, and W is null.

In another embodiment, the length of the linker is 0 to 40 chain atoms.

In another embodiment, the length of the linker is 3 to 20 chain atoms.

In another embodiment, the length of the linker is 5 to 15 chain atoms.

In another embodiment, when the CBP/P300 ligand of the bivalent compound attached to A, A is selected from —(CO)—, —(CH₂)₁₋₂(CO)—NH—, —(CH₂)₀₋₈—, —(CH₂)₀₋₃—CO—(CH₂)₀₋₈—, —(CH₂)₀₋₃—R^{r}—(CH₂)₀₋₃, —(CH₂)₀₋₃—(CO)—(CH₂)₀₋₃—R^{r}—(CH₂)₀₋₃, wherein

R^{r} is selected from Group R, and Group R is defined as in FORMULA 9.

In another embodiment, the linker is —(CO)—(CH₂)₃₋₇—.

In another embodiment, the linker is —(CH₂)₁₋₂(CO)—NH—(CH₂)₃₋₇—.

In another embodiment, the linker is —(CH₂)₀₋₁₁—, or —(CH₂)₀₋₃—CO—(CH₂)₀₋₁₀—.

In another embodiment, the linker is —(CH₂)₀₋₃—R^{r}—(CH₂)₀₋₃—, or —(CH₂)₀₋₃—(CO)—(CH₂)₀₋₃—R^{r}—(CH₂)₀₋₃—, wherein R^{r} is selected from the group Group R, and Group R is defined as in FORMULA 9.

In some embodiments, the bivalent compound is selected from the group consisting of P-001 to P-141 and CPD-1139 to CPD-1179, or a pharmaceutically acceptable salt or analog thereof.

In some embodiments, the bivalent compound is selected from the group consisting of P-004, P-005, P-006, P-007, P-015, P-020, P-026, P-027, P-033, P-034, P-035, P-036, P-041, P-043, P-085, P-088, P-090, P-091, P-093, P-096, P-097, P-100, P-104, P-106, P-109, P-110, P-111, P-112, P-113, P-115, P-116, P-119, P-120, P-129, P-130, P-131, P-133, P-135, P-142, P-143, P-146, P-147, P-148, P-149, P-151, P-153, P-155, P-157, P-159, P-160, P-161, P-162, P-163, P-164, P-166, P-173, P-174, and a pharmaceutically acceptable salt or analog thereof.

In one embodiment, the bivalent compound is 3-(7-(difluoromethyl)-6-(1-methyl-1H-pyrazol-4-yl)-3,4-dihydroquinolin-1(2H)-yl)-1-(1-(5-((2-(2,6-dioxopiperidin-3-yl)-1,3-dioxoisoindolin-5-yl)amino)pentanoyl)piperidin-4-yl)-N-methyl-1,4,6,7-tetrahydro-5H-pyrazolo[4,3-c]pyridine-5-carboxamide (P-004).

In one embodiment, the bivalent compound is 3-(7-(difluoromethyl)-6-(1-methyl-1H-pyrazol-4-yl)-3,4-dihydroquinolin-1(2H)-yl)-1-(1-(6-((2-(2,6-dioxopiperidin-3-yl)-1,3-dioxoisoindolin-5-yl)amino)hexanoyl)piperidin-4-yl)-N-methyl-1,4,6,7-tetrahydro-5H-pyrazolo[4,3-c]pyridine-5-carboxamide (P-005).

In one embodiment, the bivalent compound is 3-(7-(difluoromethyl)-6-(1-methyl-1H-pyrazol-4-yl)-3,4-dihydroquinolin-1(2H)-yl)-1-(1-(7-((2-(2,6-dioxopiperidin-3-yl)-1,3-dioxoisoindolin-5-yl)amino)heptanoyl)piperidin-4-yl)-N-methyl-1,4,6,7-tetrahydro-5H-pyrazolo[4,3-c]pyridine-5-carboxamide (P-006).

In one embodiment, the bivalent compound is 3-(7-(difluoromethyl)-6-(1-methyl-1H-pyrazol-4-yl)-3,4-dihydroquinolin-1(2H)-yl)-1-(1-(8-((2-(2,6-dioxopiperidin-3-yl)-1,3-dioxoisoindolin-5-yl)amino)octanoyl)piperidin-4-yl)-N-methyl-1,4,6,7-tetrahydro-5H-pyrazolo[4,3-c]pyridine-5-carboxamide (P-007).

In one embodiment, the bivalent compound is 3-(7-(difluoromethyl)-6-(1-methyl-1H-pyrazol-4-yl)-3,4-dihydroquinolin-1(2H)-yl)-1-(1-(4-((2-(2,6-dioxopiperidin-3-yl)-1,3-dioxoisoindolin-4-yl)amino)butanoyl)piperidin-4-yl)-N-methyl-1,4,6,7-tetrahydro-5H-pyrazolo[4,3-c]pyridine-5-carboxamide (P-015).

In one embodiment, the bivalent compound is 3-(7-(difluoromethyl)-6-(1-(2-(((6-((2-(2,6-dioxopiperidin-3-yl)-1,3-dioxoisoindolin-5-yl)amino)hexyl)amino)-2-oxoethyl)-1H-pyrazol-4-yl)-3,4-dihydroquinolin-1(2H)-yl)-N-methyl-1-(tetrahydro-2H-pyran-4-yl)-1,4,6,7-tetrahydro-5H-pyrazolo[4,3-c]pyridine-5-carboxamide (P-020).

In one embodiment, the bivalent compound is 3-(7-(difluoromethyl)-6-(1-(2-((7-((2-(2,6-dioxopiperidin-3-yl)-1,3-dioxoisoindolin-5-yl)amino)heptyl)amino)-2-oxoethyl)-1H-pyrazol-4-yl)-3,4-dihydroquinolin-1(2H)-yl)-N-methyl-1-(tetrahydro-2H-pyran-4-yl)-1,4,6,7-tetrahydro-5H-pyrazolo[4,3-c]pyridine-5-carboxamide (P-026).

In one embodiment, the bivalent compound is 3-(7-(difluoromethyl)-6-(1-(2-((8-((2-(2,6-dioxopiperidin-3-yl)-1,3-dioxoisoindolin-5-yl)amino)octyl)amino)-2-oxoethyl)-1H-pyrazol-4-yl)-3,4-dihydroquinolin-1(2H)-yl)-N-methyl-1-(tetrahydro-2H-pyran-4-yl)-1,4,6,7-tetrahydro-5H-pyrazolo[4,3-c]pyridine-5-carboxamide (P-027).

In one embodiment, the bivalent compound is 3-(7-(difluoromethyl)-6-(1-methyl-1H-pyrazol-4-yl)-3,4-dihydroquinolin-1(2H)-yl)-1-(1-(5-((2-(2,6-dioxopiperidin-3-yl)-1,3-dioxoisoindolin-4-yl)amino)pentanoyl)piperidin-4-yl)-N-methyl-1,4,6,7-tetrahydro-5H-pyrazolo[4,3-c]pyridine-5-carboxamide (P-033).

In one embodiment, the bivalent compound is 3-(7-(difluoromethyl)-6-(1-methyl-1H-pyrazol-4-yl)-3,4-dihydroquinolin-1(2H)-yl)-1-(1-(6-((2-(2,6-dioxopiperidin-3-yl)-1,3-dioxoisoindolin-4-yl)amino)hexanoyl)piperidin-4-yl)-N-methyl-1,4,6,7-tetrahydro-5H-pyrazolo[4,3-c]pyridine-5-carboxamide (P-034).

In one embodiment, the bivalent compound is 3-(7-(difluoromethyl)-6-(1-methyl-1H-pyrazol-4-yl)-3,4-dihydroquinolin-1(2H)-yl)-1-(1-(7-((2-(2,6-dioxopiperidin-3-yl)-1,3-dioxoisoindolin-4-yl)amino)heptanoyl)piperidin-4-yl)-N-methyl-1,4,6,7-tetrahydro-5H-pyrazolo[4,3-c]pyridine-5-carboxamide (P-035).

In one embodiment, the bivalent compound is 3-(7-(difluoromethyl)-6-(1-methyl-1H-pyrazol-4-yl)-3,4-dihydroquinolin-1(2H)-yl)-1-(1-(8-((2-(2,6-dioxopiperidin-3-yl)-1,3-dioxoisoindolin-4-yl)amino)octanoyl)piperidin-4-yl)-N-methyl-1,4,6,7-tetrahydro-5H-pyrazolo[4,3-c]pyridine-5-carboxamide (P-036).

In one embodiment, the bivalent compound is 3-(7-(difluoromethyl)-6-(1-(2-((6-((2-(2,6-dioxopiperidin-3-yl)-1,3-dioxoisoindolin-4-yl)amino)hexyl)amino)-2-oxoethyl)-1H-pyrazol-4-yl)-3,4-dihydroquinolin-1(2H)-yl)-N-methyl-1-(tetrahydro-2H-pyran-4-yl)-1,4,6,7-tetrahydro-5H-pyrazolo[4,3-c]pyridine-5-carboxamide (P-041).

In one embodiment, the bivalent compound is 3-(7-(difluoromethyl)-6-(1-(2-((8-((2-(2,6-dioxopiperidin-3-yl)-1,3-dioxoisoindolin-4-yl)amino)octyl)amino)-2-oxoethyl)-1H-pyrazol-4-yl)-3,4-dihydroquinolin-1(2H)-yl)-N-methyl-1-(tetrahydro-2H-pyran-4-yl)-1,4,6,7-tetrahydro-5H-pyrazolo[4,3-c]pyridine-5-carboxamide (P-043).

In one embodiment, the bivalent compound is 3-(7-(difluoromethyl)-6-(1-methyl-1H-pyrazol-4-yl)-3,4-dihydroquinolin-1(2H)-yl)-1-(1-(7-(2-(2,6-dioxopiperidin-3-yl)-1,3-dioxoisoindolin-4-yl)heptanoyl)piperidin-4-yl)-N-methyl-1,4,6,7-tetrahydro-5H-pyrazolo[4,3-c]pyridine-5-carboxamide (P-085).

In one embodiment, the bivalent compound is 3-(7-(difluoromethyl)-6-(1-methyl-1H-pyrazol-4-yl)-3,4-dihydroquinolin-1(2H)-yl)-1-(1-(6-((2-(2,6-dioxopiperidin-3-yl)-1-oxoisoindolin-4-yl)oxy)hexanoyl)piperidin-4-yl)-N-methyl-1,4,6,7-tetrahydro-5H-pyrazolo[4,3-c]pyridine-5-carboxamide (P-088).

In one embodiment, the bivalent compound is 3-(7-(difluoromethyl)-6-(1-methyl-1H-pyrazol-4-yl)-3,4-dihydroquinolin-1(2H)-yl)-1-(1-(6-((2-(2,6-dioxopiperidin-3-yl)-1,3-dioxoisoindolin-4-yl)amino)hexyl)piperidin-4-yl)-N-methyl-1,4,6,7-tetrahydro-5H-pyrazolo[4,3-c]pyridine-5-carboxamide (P-090).

In one embodiment, the bivalent compound is 3-(7-(difluoromethyl)-6-(1-methyl-1H-pyrazol-4-yl)-3,4-dihydroquinolin-1(2H)-yl)-1-(1-(6-((2-(2,6-dioxopiperidin-3-yl)-1,3-dioxoisoindolin-4-yl)oxy)hexyl)piperidin-4-yl)-N-methyl-1,4,6,7-tetrahydro-5H-pyrazolo[4,3-c]pyridine-5-carboxamide (P-091).

In one embodiment, the bivalent compound is 3-(7-(difluoromethyl)-6-(1-methyl-1H-pyrazol-4-yl)-3,4-dihydroquinolin-1(2H)-yl)-1-(1-(7-(2-(2,6-dioxopiperidin-3-yl)-1-oxoisoindolin-4-yl)hept-6-ynoyl)piperidin-4-yl)-N-methyl-1,4,6,7-tetrahydro-5H-pyrazolo[4,3-c]pyridine-5-carboxamide (P-093).

In one embodiment, the bivalent compound is 3-(7-(difluoromethyl)-6-(1-methyl-1H-pyrazol-4-yl)-3,4-dihydroquinolin-1(2H)-yl)-1-(1-(6-((2-(2,6-dioxopiperidin-3-yl)-3-oxoisoindolin-4-yl)amino)hexanoyl)piperidin-4-yl)-N-methyl-1,4,6,7-tetrahydro-5H-pyrazolo[4,3-c]pyridine-5-carboxamide (P-096).

In one embodiment, the bivalent compound is 3-(7-(difluoromethyl)-6-(1-methyl-1H-pyrazol-4-yl)-3,4-dihydroquinolin-1(2H)-yl)-1-(1-(7-(2-(2,6-dioxopiperidin-3-yl)-1,3-dioxoisoindolin-4-yl)heptyl)piperidin-4-yl)-N-methyl-1,4,6,7-tetrahydro-5H-pyrazolo[4,3-c]pyridine-5-carboxamide (P-097).

In one embodiment, the bivalent compound is 4-((6-(4-(5-acetyl-3-(7-(difluoromethyl)-6-(1-methyl-1H-pyrazol-4-yl)-3,4-dihydroquinolin-1(2H)-yl)-4,5,6,7-tetrahydro-1H-pyrazolo[4,3-c]pyridin-1-yl)piperidin-1-yl)-6-oxohexyl)amino)-2-(2,6-dioxopiperidin-3-yl)isoindoline-1,3-dione (P-100).

In one embodiment, the bivalent compound is 3-(7-(difluoromethyl)-6-(1-methyl-1H-pyrazol-4-yl)-3,4-dihydroquinolin-1(2H)-yl)-1-(1-(2-(4-(2-(2-(2,6-dioxopiperidin-3-yl)-1,3-dioxoisoindolin-4-yl)ethyl)piperidin-1-yl)acetyl)piperidin-4-yl)-N-methyl-1,4,6,7-tetrahydro-5H-pyrazolo[4,3-c]pyridine-5-carboxamide (P-104).

In one embodiment, the bivalent compound is 3-(7-(difluoromethyl)-6-(1-methyl-1H-pyrazol-4-yl)-3,4-dihydroquinolin-1(2H)-yl)-1-(1-(7-(3-(2,6-dioxopiperidin-3-yl)-2-oxo-2,3-dihydro-1H-benzo[d]imidazol-1-yl)heptanoyl)piperidin-4-yl)-N-methyl-1,4,6,7-tetrahydro-5H-pyrazolo[4,3-c]pyridine-5-carboxamide (P-106).

In one embodiment, the bivalent compound is 3-(7-(difluoromethyl)-6-(1-methyl-1H-pyrazol-4-yl)-3,4-dihydroquinolin-1(2H)-yl)-1-(1-(6-((1-(2,6-dioxopiperidin-3-yl)-3-methyl-2-oxo-2,3-dihydro-1H-benzo[d]imidazol-4-yl)amino)hexanoyl)piperidin-4-yl)-N-methyl-1,4,6,7-tetrahydro-5H-pyrazolo[4,3-c]pyridine-5-carboxamide (P-109).

In one embodiment, the bivalent compound is 3-(4-((6-(4-(5-acetyl-3-(7-(difluoromethyl)-6-(1-methyl-1H-pyrazol-4-yl)-3,4-dihydroquinolin-1(2H)-yl)-4,5,6,7-tetrahydro-1H-pyrazolo[4,3-c]pyridin-1-yl)piperidin-1-yl)-6-oxohexyl)amino)-3-methyl-2-oxo-2,3-dihydro-1H-benzo[d]imidazol-1-yl)piperidine-2,6-dione (P-110).

In one embodiment, the bivalent compound is 3-(4-((7-(4-(5-acetyl-3-(7-(difluoromethyl)-6-(1-methyl-1H-pyrazol-4-yl)-3,4-dihydroquinolin-1(2H)-yl)-4,5,6,7-tetrahydro-1H-pyrazolo[4,3-c]pyridin-1-yl)piperidin-1-yl)-7-oxoheptyl)amino)-3-methyl-2-oxo-2,3-dihydro-1H-benzo[d]imidazol-1-yl)piperidine-2,6-dione (P-111).

In one embodiment, the bivalent compound is 4-((6-(4-(5-acetyl-3-(7-(difluoromethyl)-6-(1-methyl-1H-pyrazol-4-yl)-3,4-dihydroquinolin-1(2H)-yl)-4,5,6,7-tetrahydro-1H-pyrazolo[4,3-c]pyridin-1-yl)piperidin-1-yl)hexyl)amino)-2-(2,6-dioxopiperidin-3-yl)isoindoline-1,3-dione (P-112).

In one embodiment, the bivalent compound is 4-((6-(4-(5-acetyl-3-(7-(difluoromethyl)-6-(1-methyl-1H-pyrazol-4-yl)-3,4-dihydroquinolin-1(2H)-yl)-4,5,6,7-tetrahydro-1H-pyrazolo[4,3-c]pyridin-1-yl)piperidin-1-yl)hexyl)oxy)-2-(2,6-dioxopiperidin-3-yl)isoindoline-1,3-dione (P-113).

In one embodiment, the bivalent compound is 3-(7-(difluoromethyl)-6-(1-methyl-1H-pyrazol-4-yl)-3,4-dihydroquinolin-1(2H)-yl)-1-(1-(6-((1-(2,6-dioxopiperidin-3-yl)-3-methyl-2-oxo-2,3-dihydro-1H-benzo[d]imidazol-5-yl)amino)hexanoyl)piperidin-4-yl)-N-methyl-1,4,6,7-tetrahydro-5H-pyrazolo[4,3-c]pyridine-5-carboxamide (P-115).

In one embodiment, the bivalent compound is 3-(5-((6-(4-(5-acetyl-3-(7-(difluoromethyl)-6-(1-methyl-1H-pyrazol-4-yl)-3,4-dihydroquinolin-1(2H)-yl)-4,5,6,7-tetrahydro-1H-pyrazolo[4,3-c]pyridin-1-yl)piperidin-1-yl)-6-oxohexyl)amino)-3-methyl-2-oxo-2,3-dihydro-1H-benzo[d]imidazol-1-yl)piperidine-2,6-dione (P-116).

In one embodiment, the bivalent compound is 5-((8-(4-(5-acetyl-3-(7-(difluoromethyl)-6-(1-methyl-1H-pyrazol-4-yl)-3,4-dihydroquinolin-1(2H)-yl)-4,5,6,7-tetrahydro-1H-pyrazolo[4,3-c]pyridin-1-yl)piperidin-1-yl)-8-oxooctyl)amino)-2-(2,6-dioxopiperidin-3-yl)isoindoline-1,3-dione (P-119).

In one embodiment, the bivalent compound is 5-((6-(4-(5-acetyl-3-(7-(difluoromethyl)-6-(1-methyl-1H-pyrazol-4-yl)-3,4-dihydroquinolin-1(2H)-yl)-4,5,6,7-tetrahydro-1H-pyrazolo[4,3-c]pyridin-1-yl)piperidin-1-yl)-6-oxohexyl)amino)-2-(2,6-dioxopiperidin-3-yl)isoindoline-1,3-dione (P-120).

In one embodiment, the bivalent compound is 3-(7-(difluoromethyl)-6-(1-methyl-1H-pyrazol-4-yl)-3,4-dihydroquinolin-1(2H)-yl)-1-(1-(7-((1-(2,6-dioxopiperidin-3-yl)-3-methyl-2-oxo-2,3-dihydro-1H-benzo[d]imidazol-5-yl)amino)heptanoyl)piperidin-4-yl)-N-methyl-1,4,6,7-tetrahydro-5H-pyrazolo[4,3-c]pyridine-5-carboxamide (P-129).

In one embodiment, the bivalent compound is 3-(5-((7-(4-(5-acetyl-3-(7-(difluoromethyl)-6-(1-methyl-1H-pyrazol-4-yl)-3,4-dihydroquinolin-1(2H)-yl)-4,5,6,7-tetrahydro-1H-pyrazolo[4,3-c]pyridin-1-yl)piperidin-1-yl)-7-oxoheptyl)amino)-3-methyl-2-oxo-2,3-dihydro-1H-benzo[d]imidazol-1-yl)piperidine-2,6-dione (P-130).

In one embodiment, the bivalent compound is 4-(2-(1-(2-(4-(5-acetyl-3-(7-(difluoromethyl)-6-(1-methyl-1H-pyrazol-4-yl)-3,4-dihydroquinolin-1(2H)-yl)-4,5,6,7-tetrahydro-1H-pyrazolo[4,3-c]pyridin-1-yl)piperidin-1-yl)-2-oxoethyl)piperidin-4-yl)ethyl)-2-(2,6-dioxopiperidin-3-yl)isoindoline-1,3-dione (P-131).

In one embodiment, the bivalent compound is 2-(4-(1-(5-acetyl-1-(tetrahydro-2H-pyran-4-yl)-4,5,6,7-tetrahydro-1H-pyrazolo[4,3-c]pyridin-3-yl)-7-(difluoromethyl)-1,2,3,4-tetrahydroquinolin-6-yl)-1H-pyrazol-1-yl)-N-(7-((2-(2,6-dioxopiperidin-3-yl)-1,3-dioxoisoindolin-5-yl)amino)heptyl)acetamide (P-133).

In one embodiment, the bivalent compound is 3-(3-(8-(4-(5-acetyl-3-(7-(difluoromethyl)-6-(1-methyl-1H-pyrazol-4-yl)-3,4-dihydroquinolin-1(2H)-yl)-4,5,6,7-tetrahydro-1H-pyrazolo[4,3-c]pyridin-1-yl)piperidin-1-yl)-8-oxooctyl)-2-oxo-2,3-dihydro-1H-benzo[d]imidazol-1-yl)piperidine-2,6-dione (P-135).

In one embodiment, the bivalent compound is 4-(((4-(2-(4-(5-acetyl-3-(7-(difluoromethyl)-6-(1-methyl-1H-pyrazol-4-yl)-3,4-dihydroquinolin-1(2H)-yl)-4,5,6,7-tetrahydro-1H-pyrazolo[4,3-c]pyridin-1-yl)piperidin-1-yl)-2-oxoethyl)morpholin-2-yl)methyl)amino)-2-(2,6-dioxopiperidin-3-yl)isoindoline-1,3-dione (P-142).

In one embodiment, the bivalent compound is 4-(((1-(2-(4-(5-acetyl-3-(7-(difluoromethyl)-6-(1-methyl-1H-pyrazol-4-yl)-3,4-dihydroquinolin-1(2H)-yl)-4,5,6,7-tetrahydro-1H-pyrazolo[4,3-c]pyridin-1-yl)piperidin-1-yl)ethyl)piperidin-4-yl)methyl)amino)-2-(2,6-dioxopiperidin-3-yl)isoindoline-1,3-dione (P-143).

In one embodiment, the bivalent compound is 4-(3-(4-(4-(5-acetyl-3-(7-(difluoromethyl)-6-(1-methyl-1H-pyrazol-4-yl)-3,4-dihydroquinolin-1(2H)-yl)-4,5,6,7-tetrahydro-1H-pyrazolo[4,3-c]pyridin-1-yl)piperidin-1-yl)-4-oxobutyl)azetidin-1-yl)-2-(2,6-dioxopiperidin-3-yl)isoindoline-1,3-dione (P-146).

In one embodiment, the bivalent compound is 4-(3-(1-(2-(4-(5-acetyl-3-(7-(difluoromethyl)-6-(1-methyl-1H-pyrazol-4-yl)-3,4-dihydroquinolin-1(2H)-yl)-4,5,6,7-tetrahydro-1H-pyrazolo[4,3-c]pyridin-1-yl)piperidin-1-yl)-2-oxoethyl)piperidin-4-yl)propyl)-2-(2,6-dioxopiperidin-3-yl)isoindoline-1,3-dione (P-147).

In one embodiment, the bivalent compound is 4-(2-(1-(3-(4-(5-acetyl-3-(7-(difluoromethyl)-6-(1-methyl-1H-pyrazol-4-yl)-3,4-dihydroquinolin-1(2H)-yl)-4,5,6,7-tetrahydro-1H-pyrazolo[4,3-c]pyridin-1-yl)piperidin-1-yl)-3-oxopropyl)piperidin-4-yl)ethyl)-2-(2,6-dioxopiperidin-3-yl)isoindoline-1,3-dione (P-148).

In one embodiment, the bivalent compound is 4-(3-(4-((4-(5-acetyl-3-(7-(difluoromethyl)-6-(1-methyl-1H-pyrazol-4-yl)-3,4-dihydroquinolin-1(2H)-yl)-4,5,6,7-tetrahydro-1H-pyrazolo[4,3-c]pyridin-1-yl)piperidin-1-yl)methyl)piperidin-1-yl)propyl)-2-(2,6-dioxopiperidin-3-yl)isoindoline-1,3-dione (P-149).

In one embodiment, the bivalent compound is 3-(5-((6-(4-(5-acetyl-3-(7-(difluoromethyl)-6-(1-methyl-1H-pyrazol-4-yl)-3,4-dihydroquinolin-1(2H)-yl)-4,5,6,7-tetrahydro-1H-pyrazolo[4,3-c]pyridin-1-yl)piperidin-1-yl)-6-oxohexyl)amino)-4-oxobenzo[d][1,2,3]triazin-3(4H)-yl)piperidine-2,6-dione (P-151).

In one embodiment, the bivalent compound is 3-(5-((7-(4-(5-acetyl-3-(7-(difluoromethyl)-6-(1-methyl-1H-pyrazol-4-yl)-3,4-dihydroquinolin-1(2H)-yl)-4,5,6,7-tetrahydro-1H-pyrazolo[4,3-c]pyridin-1-yl)piperidin-1-yl)-7-oxoheptyl)amino)-4-oxobenzo[d] [1,2,3]triazin-3 (4H)-yl)piperidine-2,6-dione (P-153).

In one embodiment, the bivalent compound is 3-(5-((5-(4-(5-acetyl-3-(7-(difluoromethyl)-6-(1-methyl-1H-pyrazol-4-yl)-3,4-dihydroquinolin-1(2H)-yl)-4,5,6,7-tetrahydro-1H-pyrazolo[4,3-c]pyridin-1-yl)piperidin-1-yl)-5-oxopentyl)amino)-4-oxobenzo[d][1,2,3]triazin-3(4H)-yl)piperidine-2,6-dione (P-155).

In one embodiment, the bivalent compound is 4-(2-(1-(2-(4-(5-acetyl-3-(7-(difluoromethyl)-6-(1-methyl-1H-pyrazol-4-yl)-3,4-dihydroquinolin-1(2H)-yl)-4,5,6,7-tetrahydro-1H-pyrazolo[4,3-c]pyridin-1-yl)piperidin-1-yl)ethyl)piperidin-4-yl)ethyl)-2-(2,6-dioxopiperidin-3-yl)isoindoline-1,3-dione (P-157).

In one embodiment, the bivalent compound is 3-(4-((8-(4-(5-acetyl-3-(7-(difluoromethyl)-6-(1-methyl-1H-pyrazol-4-yl)-3,4-dihydroquinolin-1(2H)-yl)-4,5,6,7-tetrahydro-1H-pyrazolo[4,3-c]pyridin-1-yl)piperidin-1-yl)-8-oxooctyl)amino)-3-methyl-2-oxo-2,3-dihydro-1H-benzo[d]imidazol-1-yl)piperidine-2,6-dione (P-159).

In one embodiment, the bivalent compound is 3-(4-((9-(4-(5-acetyl-3-(7-(difluoromethyl)-6-(1-methyl-1H-pyrazol-4-yl)-3,4-dihydroquinolin-1(2H)-yl)-4,5,6,7-tetrahydro-1H-pyrazolo[4,3-c]pyridin-1-yl)piperidin-1-yl)-9-oxononyl)amino)-3-methyl-2-oxo-2,3-dihydro-1H-benzo[d]imidazol-1-yl)piperidine-2,6-dione (P-160).

In one embodiment, the bivalent compound is 3-(5-(8-(4-(5-acetyl-3-(7-(difluoromethyl)-6-(1-methyl-1H-pyrazol-4-yl)-3,4-dihydroquinolin-1(2H)-yl)-4,5,6,7-tetrahydro-1H-pyrazolo[4,3-c]pyridin-1-yl)piperidin-1-yl)-8-oxooctyl)amino)-3-methyl-2-oxo-2,3-dihydro-1H-benzo[d]imidazol-1-yl)piperidine-2,6-dione (P-161).

In one embodiment, the bivalent compound is 3-(5-((9-(4-(5-acetyl-3-(7-(difluoromethyl)-6-(1-methyl-1H-pyrazol-4-yl)-3,4-dihydroquinolin-1(2H)-yl)-4,5,6,7-tetrahydro-1H-pyrazolo[4,3-c]pyridin-1-yl)piperidin-1-yl)-9-oxononyl)amino)-3-methyl-2-oxo-2,3-dihydro-1H-benzo[d]imidazol-1-yl)piperidine-2,6-dione (P-162).

In one embodiment, the bivalent compound is 3-(3-(9-(4-(5-acetyl-3-(7-(difluoromethyl)-6-(1-methyl-1H-pyrazol-4-yl)-3,4-dihydroquinolin-1(2H)-yl)-4,5,6,7-tetrahydro-1H-pyrazolo[4,3-c]pyridin-1-yl)piperidin-1-yl)-9-oxononyl)-2-oxo-2,3-dihydro-1H-benzo[d]imidazol-1-yl)piperidine-2,6-dione (P-163).

In one embodiment, the bivalent compound is 3-(3-(10-(4-(5-acetyl-3-(7-(difluoromethyl)-6-(1-methyl-1H-pyrazol-4-yl)-3,4-dihydroquinolin-1(2H)-yl)-4,5,6,7-tetrahydro-1H-pyrazolo[4,3-c]pyridin-1-yl)piperidin-1-yl)-10-oxodecyl)-2-oxo-2,3-dihydro-1H-benzo[d]imidazol-1-yl)piperidine-2,6-dione (P-164).

In one embodiment, the bivalent compound is 3-(4-((4-((4-(5-acetyl-3-(7-(difluoromethyl)-6-(1-methyl-1H-pyrazol-4-yl)-3,4-dihydroquinolin-1(2H)-yl)-4,5,6,7-tetrahydro-1H-pyrazolo[4,3-c]pyridin-1-yl)piperidin-1-yl)methyl)benzyl)oxy)-1-oxoisoindolin-2-yl)piperidine-2,6-dione (P-166).

In one embodiment, the bivalent compound is 3-(4-((4-(2-(4-(5-acetyl-3-(7-(difluoromethyl)-6-(1-methyl-1H-pyrazol-4-yl)-3,4-dihydroquinolin-1(2H)-yl)-4,5,6,7-tetrahydro-1H-pyrazolo[4,3-c]pyridin-1-yl)piperidin-1-yl)ethyl)benzyl)oxy)-1-oxoisoindolin-2-yl)piperidine-2,6-dione (P-173).

In one embodiment, the bivalent compound is 3-(4-(4-((4-(5-acetyl-3-(7-(difluoromethyl)-6-(1-methyl-1H-pyrazol-4-yl)-3,4-dihydroquinolin-1(2H)-yl)-4,5,6,7-tetrahydro-1H-pyrazolo[4,3-c]pyridin-1-yl)piperidin-1-yl)methyl)phenethoxy)-1-oxoisoindolin-2-yl)piperidine-2,6-dione (P-174).

According to one aspect of the present disclosure, a composition disclosed herein comprises the bivalent compound or a pharmaceutically acceptable salt or analog thereof, and a pharmaceutically acceptable carrier or diluent.

According to one aspect of the present disclosure, a method of treating a CBP/P300-mediated disease disclosed herein comprises administering to a subject with a CBP/P300-mediated disease the bivalent compound or a pharmaceutically acceptable salt or analog thereof.

In one embodiment, the CBP/P300-mediated disease results from CBP/P300 expression, mutation, deletion, or fusion.

In one embodiment, the subject with the CBP/P300-mediated disease has an elevated CBP/P300 function relative to a healthy subject without the CBP/P300-mediated disease.

In one embodiment, the bivalent compound is selected from the group consisting of P-001 to P-174, and CPD-1139 to CPD-1179, or analogs thereof.

In one embodiment, the bivalent compound is administered to the subject orally, parenterally, intradermally, subcutaneously, topically, or rectally.

In one embodiment, the method further comprises administering to the subject an additional therapeutic regimen for treating cancer, inflammatory disorders, or autoimmune diseases.

In one embodiment, the additional therapeutic regimen is selected from the group consisting of surgery, chemotherapy, radiation therapy, hormone therapy, and immunotherapy.

In one embodiment, the CBP/P300-mediated cancer is selected from the group consisting of acoustic neuroma, acute leukemia, acute lymphocytic leukemia, acute myelocytic leukemia, acute T-cell leukemia, basal cell carcinoma, bile duct carcinoma, bladder cancer, brain choriocarcinoma, chronic leukemia, chronic lymphocytic leukemia, chronic myelocytic leukemia, chronic myelogenous leukemia, colon cancer, colorectal cancer, craniopharyngioma, cystadenocarcinoma, diffuse large B-cell lymphoma, dysproliferative changes, embryonal carcinoma, endometrial cancer, endotheliosarcoma, ependymoma, epithelial carcinoma, erythroleukemia, esophageal cancer, estrogen-receptor positive breast cancer, essential thrombocythemia, Ewing's tumor, fibrosarcoma, follicular lymphoma, germ cell testicular cancer, glioma, glioblastoma, gliosarcoma, heavy chain disease, head and neck cancer, hemangioblastoma, hepatoma, hepatocellular cancer, hormone insensitive prostate cancer, leiomyosarcoma, leukemia, liposarcoma, lung cancer, lymphagioendotheliosarcoma, lymphangiosarcoma, lymphoblastic leukemia, lymphoma, lymphoid malignancies of T-cell or B-cell origin, medullary carcinoma, medulloblastoma, melanoma, meningioma, mesothelioma, multiple myeloma, myelogenous leukemia, myeloma, myxosarcoma, neuroblastoma, NUT midline carcinoma (NMC), non-small cell lung cancer, oligodendroglioma, oral cancer, osteogenic sarcoma, ovarian cancer, pancreatic cancer, papillary adenocarcinomas, papillary carcinoma, pinealoma, polycythemia vera, prostate cancer, rectal cancer, renal cell carcinoma, retinoblastoma, rhabdomyosarcoma, sarcoma, sebaceous gland carcinoma, seminoma, skin cancer, small cell lung carcinoma, solid tumors (carcinomas and sarcomas), small cell lung cancer, stomach cancer, squamous cell carcinoma, synovioma, sweat gland carcinoma, thyroid cancer, Waldenstrom's macroglobulinemia, testicular tumors, uterine cancer, and Wilms' tumor.

In one embodiment, the CBP/P300-mediated cancer is selected from the group consisting of prostate cancer, lung cancer, breast cancer, pancreatic cancer, colorectal cancer, and melanoma.

In one embodiment, the CBP/P300-mediated inflammatory disorders or the autoimmune diseases are selected from the group consisting of Addison's disease, acute gout, ankylosing spondylitis, asthma, atherosclerosis, Behcet's disease, bullous skin diseases, chronic obstructive pulmonary disease, Crohn's disease, dermatitis, eczema, giant cell arteritis, fibrosis, glomerulonephritis, hepatic vascular occlusion, hepatitis, hypophysitis, immunodeficiency syndrome, inflammatory bowel disease, Kawasaki disease, lupus nephritis, multiple sclerosis, myocarditis, myositis, nephritis, organ transplant rejection, osteoarthritis, pancreatitis, pericarditis, Polyarteritis nodosa, pneumonitis, primary biliary cirrhosis, psoriasis, psoriatic arthritis, rheumatoid arthritis, scleritis, sclerosing cholangitis, sepsis, systemic lupus erythematosus, Takayasu's Arteritis, toxic shock, thyroiditis, diabetes, ulcerative colitis, uveitis, vitiligo, vasculitis, and Wegener's granulomatosis.

In one embodiment, the CBP/P300-mediated disease is a relapsed cancer.

In one embodiment, the CBP/P300-mediated disease is refractory to one or more previous treatments.

According to one aspect of the present disclosure, a method for identifying a bivalent compound which mediates degradation or reduction of CBP/P300 is disclosed. The method comprises:

providing a heterobifunctional test compound comprising an CBP/P300 ligand conjugated to a degradation tag through a linker;

contacting the heterobifunctional test compound with a cell comprising a ubiquitin ligase and CBP/P300;

determining whether CBP/P300 level is decreased in the cell; and

identifying the heterobifunctional test compound as a bivalent compound which mediates degradation or reduction of CBP/P300.

In one embodiment, the cell is a cancer cell.

In one embodiment, the cancer cell is a CBP/P300-dependent cancer cell.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 shows immunoblots of P300 protein expressed by LNCaP cells after treatment with 5 nM GNE-781 or heterobifunctional compounds P-001 to P-036.

FIG. 2 shows immunoblots of P300 protein expressed by LNCaP cells after treatment with GNE-781, P-003, P-004, P-005, P-015, P-016, or P-020 at indicated concentrations.

FIG. 3 shows an immunoblot of P300 protein expressed by LNCaP cells after treatment with GNE-781, P-004, P-005, P-015, or P-020 at various timepoints.

FIG. 4 shows a graph of LNCaP cell viability vs. concentrations of GNE-781, P-001, P-002, and P-019.

FIG. 5 shows an immunoblot of P300 and CBP protein expressed by LNCaP cells after treatment with GNE-781 or heterobifunctional compounds P-056, P-57, P-58, P-59, P-060, P-062, P-063, P-067, P-068 or P-069.

FIG. 6 shows immunoblots of P300 and CBP protein expressed by LNCaP cells after treatment with P-084 to P-093, P-096, P-097, P-100, P-102, or P-104 to P-108 at indicated concentrations.

FIG. 7A shows an immunoblot of P300 protein expressed by LNCaP cells after treatment with heterobifunctional compounds P-034 or P-034-neg at indicated concentrations.

FIG. 7B shows an immunoblot of P300 protein expressed by 22RV1 cells after treatment with heterobifunctional compounds P-034 or P-034-neg at indicated concentrations.

FIG. 8 shows an immunoblot of P300 and CBP protein expressed by LNCaP cells after treatment with 10 nM GNE-781, P-007, P-034, or P-100 in the presence or absence of pomalidomide, MG-132, Bortezomib or MLN4924.

FIG. 9 shows an immunoblot of P300 and CBP protein expressed in subcutaneous 22RV1 xenograft tumors after treatment with a single dose of 40 mg/kg P-100, P-007, or P-034 via intraperitoneal injection (i.p.) or oral gavage (p.o.).

FIG. 10 shows immunoblots of P300 and CBP protein expressed by LNCaP cells (FIG. 10A-B) or 22RV1 cells (FIG. 10C-E) after treatment with heterobifunctional compounds P-095 or P-109 to P-131 at indicated concentrations.

FIG. 11 shows immunoblots of P300 and CBP protein expressed by LNCaP cells (FIG. 11B-E) or 22RV1 cells (FIG. 11A) after treatment with heterobifunctional compounds P-142 to P-174 at indicated concentrations.

FIG. 12 shows immunoblots of CBP protein expressed in the lung tissues of ICR mice after treatment with a single dose of 40 mg/kg indicated heterobifunctional compounds via oral gavage (p.o.).

### DETAILED DESCRIPTION OF THE INVENTION

Posttranslational modifications of proteins, such as phosphorylation, acetylation, methylation, and ubiquitination, greatly contribute to the diversity and regulation of proteins. P300 (encoded by EP300) and the closely related CBP (encode by CREBBP) are two extensively studied lysine acetyltransferases (HATs) that catalyze transfer of acetyl groups to lysine residues of proteins. The best defined substrates of P300 and CBP are histones. Acetylation of histones modulates the conformation of chromatin and generally leads to transcription activation. Recruiting P300 and/or CBP is essential for many transcription factors and other transcription regulators to effectively promote regional transcription (Dancy and Cole, 2015). Substrates of P300 and CBP also include many non-histone proteins that have crucial physiological and pathological functions, such as p53, MYC, FOXO1, and NF-κB (Dancy and Cole, 2015). Because P300 and CBP functionally interact with a wide variety of signaling proteins, these two lysine acetyltransferases act as the converge point of many signal transduction pathways (Bedford et al., 2010). Through modulating acetylation of diverse substrates and connecting a multitude of binding partners, P300 and CBP are widely implicated in biological processes, such as cellular proliferation, differentiation, development, DNA repair, inflammation, metabolism, and memory.

Both P300 and CBP are indispensable for development, as mice deficient in either P300 or CBP die early during embryogenesis (Goodman and Smolik, 2000). Aberrant P300 or CBP are associated with a wide range of human diseases. Germline mutations that inactivate one of CREBBP alleles result in the Rubinstein-Taybi syndrome (Petrij et al., 1995), probably due to impaired activation of the Hedgehog family transcription factors. Both P300 and CBP are known to contribute to hematopoiesis, through interaction with hematopoietic transcription factors, such as GATA-1 (Blobel, 2000). Tumor suppressive roles of P300 and CBP have been well defined. Patients with Rubinstein-Taybi syndrome have higher cancer prevalence. Inactivating mutations of P300 and CBP are frequently found in human cancers (Giles et al., 1998). However, these two HATs also promote oncogenesis via different mechanisms. In a subset of acute myeloid leukemia, recurrent chromosomal translocations t(8; 16)(p11; p13) produce in-frame fusions of the MOZ gene and the CREBBP gene that direct expression of oncogenic MOZ-CBP fusion proteins (Rozman et al., 2004). CBP, and less frequently P300, are also found to fuse with MLL in chemoresistant leukemia (Sobulo et al., 1997). Accumulating evidence show that P300 and CBP are recruited as co-activators by the majority of oncogenic transcription factors, such as MYC (Faiola et al., 2005; Vervoorts et al., 2003), NF-κB (Vanden Berghe et al., 1999), β-catenin (Sun et al., 2000), E2F1 (Ianari et al., 2004; Martinez-Balbas et al., 2000), and nuclear receptors (Chakravarti et al., 1996). Hence, depleting P300 and/or CBP may compromise tumor growth through impairing the functions of these oncogenic transcription factors. Additionally, P300 has been reported to regulate immune cell functions (Liu et al., 2013). Further, P300 and CBP are important transcription co-activators for the STAT and NF-κB family transcription factors (Nadiminty et al., 2006; Wang et al., 2005; Wang et al., 2017), which have crucial functions in immune cells. Therefore, P300/CBP antagonizers may be employed to modulate activities of the immune system and the crosstalk between immune cells and cancer cells (Liu et al., 2013). Finally, it has been extensively documented that histone acetylation is crucially implicated in neurodegenerative diseases (Saha and Pahan, 2006; Valor et al., 2013). Taken together, developing novel therapeutic agents targeting P300 and CBP represents novel opportunities for the treatment of cancer, inflammatory diseases, neurological indications, and other indications.

P300 and CBP share nearly 75% similarity and 63% identity in protein sequences. Greater homology is found in functional domains that are highly conserved during evolution. Most of these domains mediate protein-protein interactions, such as the Cysteine-Histidine-rich region 1 (CH1), the CREB-interacting KIX domain, the Cysteine-Histidine-rich region (CH3), and the nuclear receptor co-activator binding domain (Wang et al., 2013a). However, these domains are less amenable to small molecule-mediated intervention. Only few inhibitors have been reported. For example, naphthol-AS-E (Uttarkar et al., 2015), compound 1-10 (Wang et al., 2013b), and MYBMIM (Ramaswamy et al., 2018) are reported as KIX domain inhibitors. KCN1 (Shi et al., 2012; Yin et al., 2012), OHM1 (Lao et al., 2014), HBS1 (Kushal et al., 2013), and KCN1 analogs (Ferguson et al., 2017) were discovered as disruptors of TAZ1/HIF-1α protein interactions. ICG-001 (Emami et al., 2004) was reported as selective inhibitor of CBP NRID/β-catenin interactions. In addition, YH249 and YH250 (Yusuke et al., 2016) were reported to selectively inhibit P300-dependent transcription. Recent efforts to develop small molecule probes for P300 and CBP are concentrated on the HAT domain and the bromodomain. The HAT domain is responsible to catalyze transfer of acetyl groups, while the bromodomain binds to acetylated lysine residues, which promotes interaction of P300 and CBP to acetylated chromatin. A variety of small molecule compounds, including GNE-781 (Bronner et al., 2017), GNE-272 (Bronner et al., 2017), GNE-207 (Lai et al., 2018), CPD 4d (Hewings et al., 2011), CPD (S)-8 (Hewings et al., 2013), CPD (R)-2 (Rooney et al., 2014), CPD6 (Unzue et al., 2016), CPD19 (Unzue et al., 2016), XDM-CBP (Hugle et al., 2017; Unzue et al., 2016), I-CBP112 (Picaud et al., 2015), TPOP146 (Popp et al., 2016), CPI-637 (Taylor et al., 2016), SGC-CBP30 (Hammitzsch et al., 2015; Hay et al., 2014), CPD 11 (Denny et al., 2017), CPD 41 (Denny et al., 2017), CPD 30 (Lai et al., 2018), CPD 5 (Bronner et al., 2017), CPD 27 (Bronner et al., 2017), CPD 29 (Bronner et al., 2017), and CCS1477 (clinical trial ID: NCT03568656), have been described to target the bromodomain of P300 and CBP HAT domain targeting P300/CBP inhibitors, C646 (Oike et al., 2014) and A-485 (Lasko et al., 2017), were reported. Transcription dependent on P300 or CBP is partially compromised by these compounds (Wei et al., 2018). These HAT or bromodomain inhibitors have exhibited anti-cancer activities in a wide range of human cancers, including but are not limited to prostate cancer (Jin et al., 2017; Lasko et al., 2017), breast cancer (Yang et al., 2013), lung cancer (Ogiwara et al., 2016; Oike et al., 2014), acute myeloid leukemia (Giotopoulos et al., 2016), and melanoma (Wang et al., 2018). However, there are significant caveats using these small molecule inhibitors to modulate the activities of P300 and CBP. First, P300 and CBP have multiple functional domains. Blockade of either the HAT domains or the bromodomains only lead to partial inhibition of their activities. The scaffolding functions P300 and CBP are not effectively modulated by these small molecule inhibitors. Second, the HAT domains and the bromodomains of P300 and CBP share significant homology so that most small molecule compounds do not effectively differentiate these two targets. Conversely, P300 and CBP have distinct tissue type-dependent roles. For example, in prostate cancer, P300 is the dominating co-activator of androgen receptor, while CBP has limited roles (Ianculescu et al., 2012). Thus, simultaneously targeting both P300 and CBP is not always necessary and may result in more significant adverse effects than selectively targeting one of them. Not to mention many P300/CBP inhibitors have off-target effects that have been poorly defined. To improve the selectivity and activity of anti-P300/CBP therapy, approaches that selectively degrade the target protein(s) are expected to have substantial advantages.

Without wishing to be bound by any theory, the present disclosure is believed to be based, at least in part, on the discovery that novel heterobifunctional small molecules which degrade CBP/P300, CBP/P300 fusion proteins, and/or CBP/P300 mutant proteins (“PROteolysis TArgeting Chimeras”/“PROTACs” and “Specific and Nongenetic IAP-dependent Protein Erasers”/“SNIPERs”) are useful in the treatment of CBP/P300-mediated diseases, particularly prostate cancer (Jin et al., 2017; Lasko et al., 2017), breast cancer (Yang et al., 2013), lung cancer (Ogiwara et al., 2016; Oike et al., 2014), acute myeloid leukemia (Giotopoulos et al., 2016), and melanoma (Wang et al., 2018).

Selective degradation of a target protein induced by a small molecule may be achieved by recruiting an E3 ubiquitin ligase and mimicking protein misfolding with a hydrophobic tag (Buckley and Crews, 2014). Additionally, PROTACs are bivalent inhibitors having one moiety that binds to an E3 ubiquitin ligase and another moiety that binds the protein target of interest (Buckley and Crews, 2014). The induced proximity leads to ubiquitination of the target followed by its degradation via proteasome-mediated proteolysis. Several types of high affinity small-molecule E3 ligase ligands have been identified or developed. They include (1) immunomodulatory drugs (IMiDs) such as thalidomide and pomalidomide, which bind cereblon (CRBN or CRL4CRBN), a component of a cullin-RING ubiquitin ligase (CRL) complex (Bondeson et al., 2015; Chamberlain et al., 2014; Fischer et al., 2014; Ito et al., 2010; Winter et al., 2015); (2) VHL-1, a hydroxyproline-containing ligand, which binds van Hippel-Lindau protein (VHL or CRL2VHL), a component of another CRL complex (Bondeson et al., 2015; Buckley et al., 2012a; Buckley et al., 2012b; Galdeano et al., 2014; Zengerle et al., 2015); (3) compound 7, which selectively binds KEAP1, a component of a CRL3 complex (Davies et al., 2016); (4) AMG232, which selectively binds MDM2, a heterodimeric RING E3 ligase (Sun et al., 2014); and (5) LCL161, which selectively binds IAP, a homodimeric RING E3 ligase (Ohoka et al., 2017; Okuhira et al., 2011; Shibata et al., 2017). The PROTAC technology has been applied to degradation of several protein targets (Bondeson et al., 2015; Buckley et al., 2015; Lai et al., 2016; Lu et al., 2015; Winter et al., 2015; Zengerle et al., 2015). In addition, a hydrophobic tagging approach, which utilizes a bulky and hydrophobic adamantyl group, has been developed to mimic protein misfolding, leading to the degradation of the target protein by proteasome (Buckley and Crews, 2014). This approach has been applied to selective degradation of the pseudokinase HER3 (Xie et al., 2014). The inventors have not yet seen any efforts applying any of these approaches to degradation of CBP/P300, CBP/P300 mutant, CBP/P300 deletion, or CBP/P300 fusion proteins.

Currently available small molecules targeting CBP/P300 focus on inhibition of the protein interactions or acetyltransferase activities of CBP/P300. A number of selective small-molecule CBP/P300 inhibitors, such as GNE-781, GNE-272, GNE-207, CPD 4d, CPD (S)-8, CPD (R)-2, CPD6, CPD19, XDM-CBP, I-CBP112, TPOP146, CPI-637, SGC-CBP30, CPD 11, CPD 41, CPD 30, CPD 5, CPD 27, CPD 29, CCS1477 (clinical trial ID: NCT03568656), C646 (Oike et al., 2014), A-485, naphthol-AS-E (Uttarkar et al., 2015), compound 1-10 (Wang et al., 2013b), MYBMIM (Ramaswamy et al., 2018), KCN1 (Shi et al., 2012; Yin et al., 2012), OHM1 (Lao et al., 2014), HBS1 (Kushal et al., 2013), and KCN1 analogs (Ferguson et al., 2017), ICG-001 (Emami et al., 2004), YH249 (Yusuke et al., 2016) and YH250 (Yusuke et al., 2016) have been reported.

In the present disclosure, a novel approach is taken: to develop compounds that directly and selectively modulate not only the protein-protein interactions and acetyltransferase activity of CBP/P300, but also their protein levels. Strategies for inducing protein degradation include recruiting E3 ubiquitin ligases, mimicking protein misfolding with hydrophobic tags, and inhibiting chaperones. Such an approach, based on the use of bivalent small molecule compounds, permits more flexible regulation of protein levels in vitro and in vivo compared with techniques such as gene knockout or short hairpin RNA-mediated (shRNA) knockdown. Unlike gene knockout or shRNA knockdown, a small molecule approach further provides an opportunity to study dose and time dependency in a disease model through modulating the administration routes, concentrations and frequencies of administration of the corresponding small molecule.

Bivalent Compounds

In some aspects, the present disclosure provides bivalent compounds including a CBP/P300 ligand conjugated to a degradation tag, or a pharmaceutically acceptable salt or analog thereof. The CBP/P300 ligand may be conjugated to the degradation tag directly or via a linker moiety. In certain embodiments, the CBP/P300 ligand may be conjugated to the degradation tag directly. In certain embodiments, the CBP/P300 ligand may be conjugated to the degradation tag via a linker moiety.

As used herein, the terms “cyclic-AMP response element binding protein and/or adenoviral E1A binding protein of 300 kDa” and “CBP/P300 ligand”, or “CBP/P300 targeting moiety” are to be construed to encompass any molecules ranging from small molecules to large proteins that associate with or bind to CBP and/or P300 proteins. In certain embodiments, the CBP/P300 ligand is capable of binding to a CBP/P300 protein comprising CBP/P300, a CBP/P300 mutant, a CBP/P300 deletion, or a CBP/P300 fusion protein. The CBP/P300 ligand can be, for example but not limited to, a small molecule compound (i.e., a molecule of molecular weight less than about 1.5 kilodaltons (kDa)), a peptide or polypeptide, nucleic acid or oligonucleotide, carbohydrate such as oligosaccharides, or an antibody or fragment thereof.

CBP/P300 Ligand

The CBP/P300 ligand or targeting moiety can be a CBP/P300 inhibitor or a portion of CBP/P300 inhibitor. In certain embodiments, the CBP/P300 inhibitor comprises one or more of (e.g., GNE-781, GNE-272, GNE-207, CPD 4d, CPD (S)-8, CPD (R)-2, CPD6, CPD19, XDM-CBP, I-CBP112, TPOP146, CPI-637, SGC-CBP30, CPD 11, CPD 41, CPD 30, CPD 5, CPD 27, CPD 29, CCS1477 (clinical trial ID: NCT03568656), C646 (Oike et al., 2014), A-485, naphthol-AS-E (Uttarkar et al., 2015), compound 1-10 (Wang et al., 2013b), MYBMIM (Ramaswamy et al., 2018), KCN1 (Shi et al., 2012; Yin et al., 2012), OHM1 (Lao et al., 2014), HBS1 (Kushal et al., 2013), and KCN1 analogs (Ferguson et al., 2017), ICG-001 (Emami et al., 2004), YH249 (Yusuke et al., 2016) and YH250 (Yusuke et al., 2016), and analogs thereof), which is capable of inhibiting the protein-protein interaction or acetyltransferase activity of CBP/P300. As used herein, a “CBP/P300 inhibitor” refers to an agent that restrains, retards, or otherwise causes inhibition of a physiological, chemical or enzymatic action or function and causes a decrease in binding of at least 5%. An inhibitor can also or alternately refer to a drug, compound, or agent that prevents or reduces the expression, transcription, or translation of a gene or protein. An inhibitor can reduce or prevent the function of a protein, e.g., by binding to or activating/inactivating another protein or receptor.

In certain embodiments, the CBP/P300 ligand is derived from a CBP/P300 inhibitor comprising:

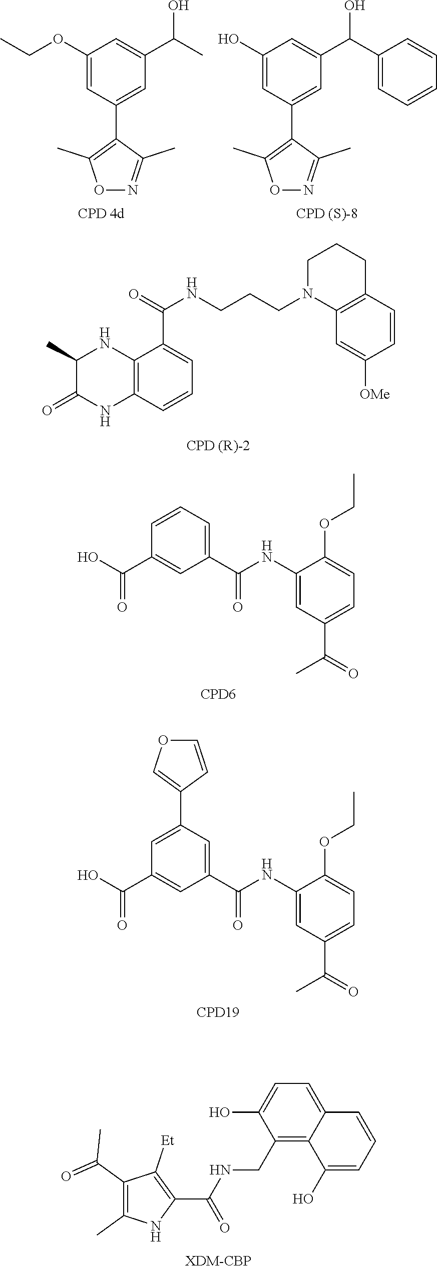

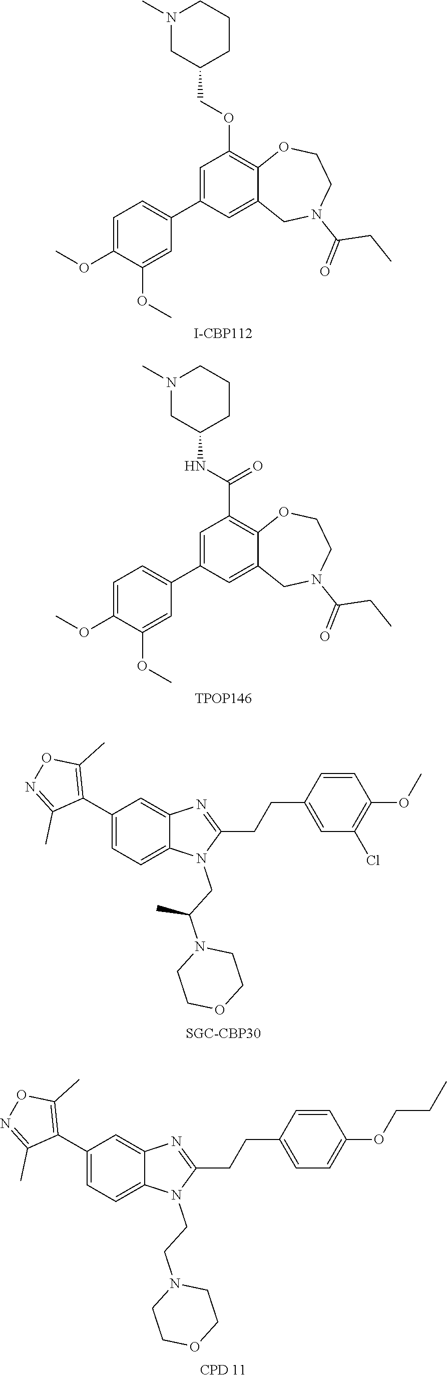

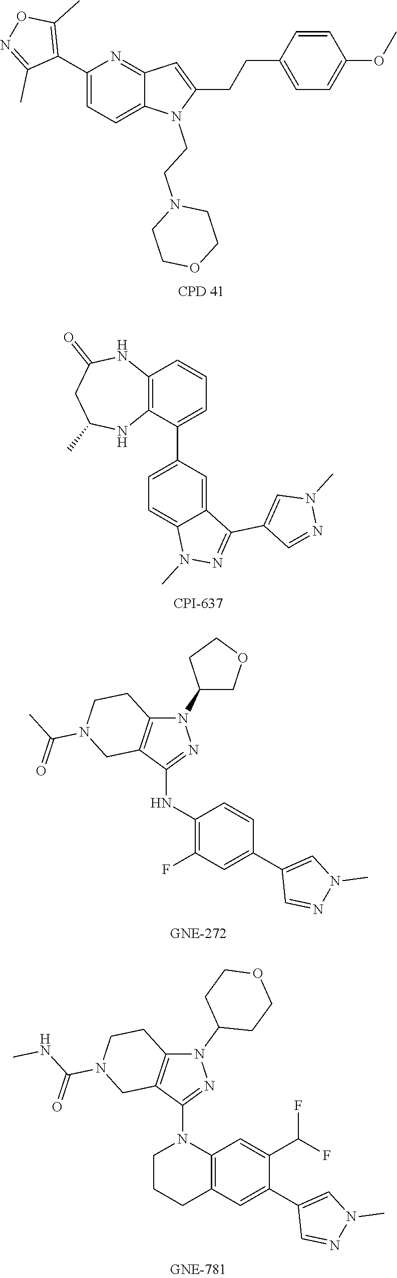

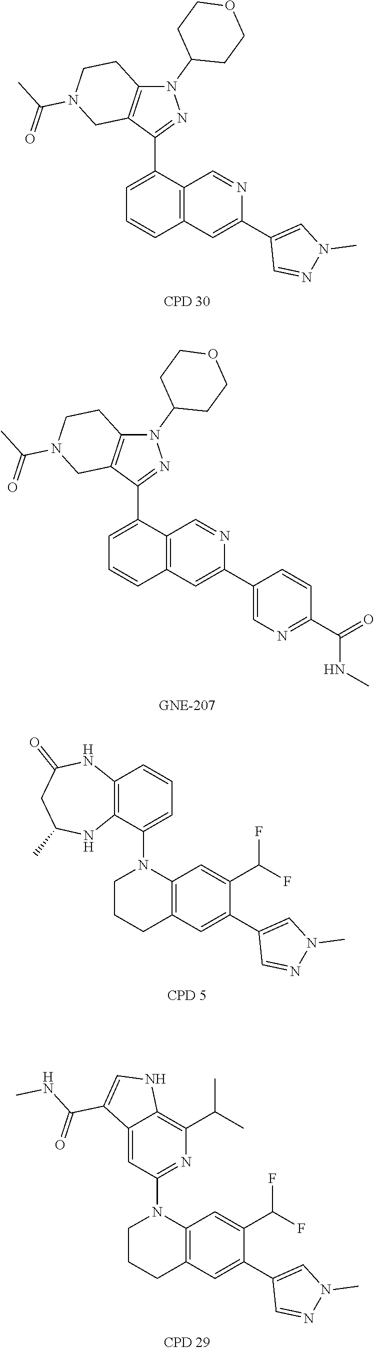

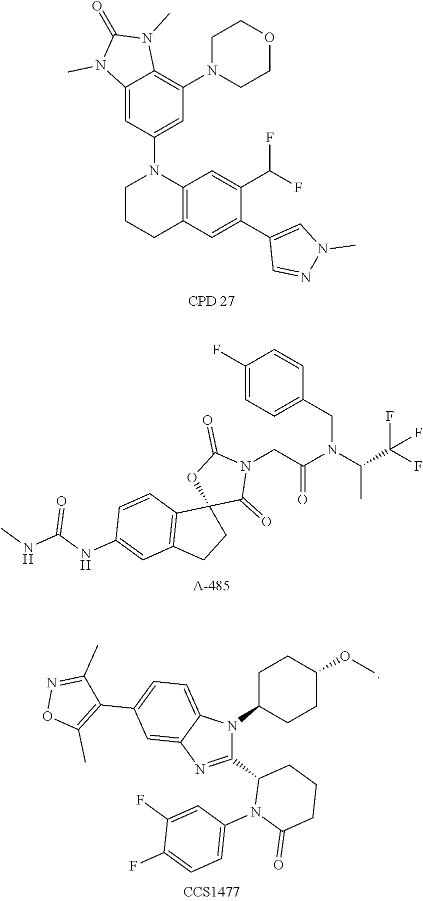

In certain embodiments, the CBP/P300 ligand include, but are not limited to GNE-781, GNE-272, GNE-207, CPD 4d, CPD (S)-8, CPD (R)-2, CPD6, CPD19, XDM-CBP, I-CBP112, TPOP146, CPI-637, SGC-CBP30, CPD 11, CPD 41, CPD 30, CPD 5, CPD 27, CPD 29, CCS1477 (clinical trial ID: NCT03568656), C646 (Oike et al., 2014), A-485, naphthol-AS-E (Uttarkar et al., 2015), compound 1-10 (Wang et al., 2013b), MYBMIM (Ramaswamy et al., 2018), KCN1 (Shi et al., 2012; Yin et al., 2012), OHM1 (Lao et al., 2014), HBS1 (Kushal et al., 2013), and KCN1 analogs (Ferguson et al., 2017), ICG-001 (Emami et al., 2004), YH249 (Yusuke et al., 2016) and YH250 (Yusuke et al., 2016).

In another embodiment, the CBP/P300 ligand comprises a moiety of FORMULA 1:

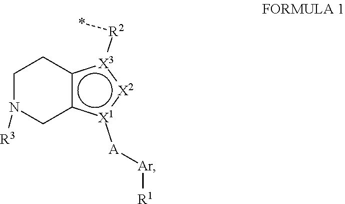

wherein

the linker moiety of the bivalent compound is attached to R²;

X¹ and X³ are independently selected from C and N, with the proviso that at least one of X¹ and X³ is C and at most only one of X¹ and X³ is N;

X² is selected from CR′, O, and NR′, wherein

R′ is selected from H, optionally substituted C₁-C₈ alkyl, and optionally substituted 3-10 membered carbocyclyl;

A is selected from null, CR⁴R⁵, CO, O, S, SO, SO₂, and NR⁴, wherein

R⁴ and R⁵ are independently selected from hydrogen, halogen, hydroxyl, cyano, nitro, optionally substituted C₁-C₈ alkyl, optionally substituted C₂-C₈ alkenyl, optionally substituted C₂-C₈ alkynyl, optionally substituted C₁-C₈ alkoxy, optionally substituted C₁-C₈alkoxyC₁-C₈alkyl, optionally substituted C₁-C₈ alkylamino, optionally substituted C₁-C₈alkylaminoC₁-C₈alkyl, optionally substituted 3-10 membered carbocyclyl, optionally substituted 3-8 membered cycloalkoxy, optionally substituted 3-10 membered carbocyclylamino, optionally substituted 4-10 membered heterocyclyl, optionally substituted aryl, and optionally substituted heteroaryl;

Ar and R⁴, Ar and R⁵, and/or R⁴ and R⁵ together with the atom to which they are connected form an optionally substituted 3-20 membered cycloalkyl or heterocyclyl ring;

Ar is selected from aryl, heteroaryl, bicyclic aryl, bicyclic heteroaryl, tricyclic aryl, tricyclic heteroaryl groups, each of which is substituted with R¹ and optionally substituted with one or more substituents independently selected from hydrogen, halogen, oxo, CN, NO₂, OR⁶, SR⁶, NR⁶R⁷, OCOR⁶, OCO₂R⁶, OCONR⁶R⁷, COR⁶, CO₂R⁶, CONR⁶R⁷, SOR⁶, SO₂R⁶, SO₂NR⁶R⁷, NR⁸CO₂R⁶, NR⁸COR⁶, NR⁸C(O)NR⁶R⁷, NR⁸SOR⁶, NR⁸SO₂R⁶, NR⁸SO₂NR⁶R⁷, optionally substituted C₁-C₈ alkyl, optionally substituted C₂-C₈ alkenyl, optionally substituted C₂-C₈ alkynyl, optionally substituted C₁-C₈ alkoxy, optionally substituted C₁-C₈alkoxyC₁-C₈alkyl, optionally substituted C₁-C₈alkylamino, optionally substituted C₁-C₈alkylaminoC₁-C₈alkyl, optionally substituted 3-10 membered carbocyclyl, optionally substituted 3-8 membered cycloalkoxy, optionally substituted 3-10 membered carbocyclylamino, optionally substituted 4-10 membered heterocyclyl, optionally substituted aryl, and optionally substituted heteroaryl, wherein

R⁶, R⁷, and R⁸ are independently selected from hydrogen, optionally substituted C₁-C₈ alkyl, optionally substituted C₂-C₈ alkenyl, optionally substituted C₂-C₈ alkynyl, optionally substituted C₁-C₈alkoxyC₁-C₈alkyl, optionally substituted C₁-C₈alkylaminoC₁-C₈alkyl, optionally substituted 3-10 membered carbocyclyl, optionally substituted 4-10 membered heterocyclyl, optionally substituted aryl, and optionally substituted heteroaryl, or

R⁶ and R⁷, R⁶ and R⁸ together with the atom to which they are connected form a 4-20 membered heterocyclyl ring;

R¹ is selected from hydrogen, halogen, CN, NO₂, OR⁹, SR⁹, NR⁹R¹⁰, OCOR⁹, OCO₂R⁹, OCONR⁹R¹⁰, COR⁹, CO₂R⁹, CONR⁹R¹⁰, SOR⁹, SO₂R⁹, SO₂NR⁹R¹⁰, NR¹¹CO₂R⁹, NR¹¹COR⁹, NR¹¹C(O)NR⁹R¹⁰, NR¹¹SOR⁹, NR¹¹SO₂R⁹, NR¹¹SO₂NR⁹R¹⁰, optionally substituted C₁-C₈ alkyl, optionally substituted C₂-C₈ alkenyl, optionally substituted C₂-C₈ alkynyl, optionally substituted C₁-C₈ alkoxy, optionally substituted C₁-C₈alkoxyC₁-C₈alkyl, optionally substituted C₁-C₈alkylamino, optionally substituted C₁-C₈alkylaminoC₁-C₈alkyl, optionally substituted 3-10 membered carbocyclyl, optionally substituted 3-8 membered cycloalkoxy, optionally substituted 3-10 membered carbocyclylamino, optionally substituted 4-10 membered heterocyclyl, optionally substituted aryl, and optionally substituted heteroaryl, wherein

R⁹, R¹⁰, and R¹¹ are independently selected from hydrogen, optionally substituted C₁-C₈ alkyl, optionally substituted C₂-C₈ alkenyl, optionally substituted C₂-C₈ alkynyl, optionally substituted C₁-C₈alkoxyC₁-C₈alkyl, optionally substituted C₁-C₈alkylaminoC₁-C₈alkyl, optionally substituted 3-10 membered carbocyclyl, optionally substituted 4-10 membered heterocyclyl, optionally substituted aryl, and optionally substituted heteroaryl, or

R⁹ and R¹⁰, R⁹ and R¹¹ together with the atom to which they are connected form a 3-20 membered cycloalkyl or 4-20 membered heterocyclyl ring;

R² is connected to the “linker” moiety of the bivalent compound, and is selected from null, R″O, R″S, R″NR¹², R″OC(O), R″OC(O)O, R″OCONR¹², R″C(O), R″C(O)O, R″CONR¹², R″S(O), R″S(O)₂, R″SO₂NR¹², R″NR¹³C(O)O, R″NR¹³C(O), R″NR¹³C(O)NR¹², R″NR¹³S(O), R″NR¹³S(O)₂, R″NR¹³S(O)₂NR¹², optionally substituted C₁-C₈ alkylene, optionally substituted C₂-C₈ alkenylene, optionally substituted C₂-C₈ alkynylene, optionally substituted 3-10 membered carbocyclyl, optionally substituted 4-10 membered heterocyclyl, optionally substituted C₃-C₁₃ fused cycloalkyl, optionally substituted C₃-C₁₃ fused heterocyclyl, optionally substituted C₃-C₁₃ bridged cycloalkyl, optionally substituted C₃-C₁₃ bridged heterocyclyl, optionally substituted C₃-C₁₃ spiro cycloalkyl, optionally substituted C₃-C₁₃ spiro heterocyclyl, optionally substituted aryl, and optionally substituted heteroaryl, wherein

R″ is null, or a bivalent moiety selected from optionally substituted C₁-C₈ alkylene, optionally substituted C₂-C₈ alkenylene, optionally substituted C₂-C₈ alkynylene, optionally substituted C₁-C₈alkoxyC₁-C₈alkylene, optionally substituted C₁-C₈ haloalkylene, optionally substituted C₁-C₈ hydroxyalkylene, optionally substituted 3-10 membered carbocyclyl, optionally substituted 4-10 membered heterocyclyl, optionally substituted C₃-C₁₃ fused cycloalkyl, optionally substituted C₃-C₁₃ fused heterocyclyl, optionally substituted C₃-C₁₃ bridged cycloalkyl, optionally substituted C₃-C₁₃ bridged heterocyclyl, optionally substituted C₃-C₁₃ spiro cycloalkyl, optionally substituted C₃-C₁₃ spiro heterocyclyl, optionally substituted aryl, and optionally substituted heteroaryl;

R¹² and R¹³ are independently selected from optionally substituted C₁-C₈ alkyl, optionally substituted C₂-C₈ alkenyl, optionally substituted C₂-C₈ alkynyl, optionally substituted C₁-C₈alkoxyC₁-C₈alkyl, optionally substituted C₁-C₈alkylaminoC₁-C₈alkyl, optionally substituted 3-10 membered carbocyclyl, optionally substituted 4-10 membered heterocyclyl, optionally substituted aryl, and optionally substituted heteroaryl, or

R¹² and R¹³ together with the atom to which they are connected form a 3-20 membered cycloalkyl or 4-20 membered heterocyclyl ring; and

R³ is selected from hydrogen, COR¹⁴, CO₂R¹⁴, CONR¹⁴R¹⁵, SOR¹⁴, SO₂R¹⁴, SO₂NR¹⁴R¹⁵, optionally substituted C₁-C₆ alkyl, optionally substituted C₂-C₆ alkenyl, optionally substituted C₂-C₆ alkynyl, optionally substituted 3-6 membered cycloalkyl, optionally substituted 4-6 membered heterocyclyl, optionally substituted aryl, and optionally substituted heteroaryl, wherein

R¹⁴ and R¹⁵ are independently selected from hydrogen, optionally substituted C₁-C₆ alkyl, optionally substituted C₂-C₆ alkenyl, optionally substituted C₂-C₆ alkynyl, optionally substituted 3-6 membered cycloalkyl, optionally substituted 4-6 membered heterocyclyl, optionally substituted aryl, and optionally substituted heteroaryl, or

R¹⁴ and R¹⁵ together with the atom to which they are connected form a 4-20 membered heterocyclyl ring.

In another embodiment, X¹ is C; and X² and X³ are N. The FORMULA I is FORMULA 1A:

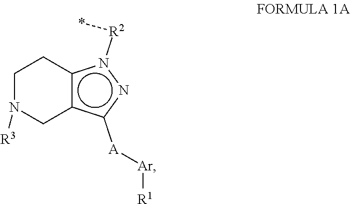

wherein

A, Ar, R¹, R² and R³ are the same as in FORMULA 1.

In another embodiment, A-Ar—R¹ is a moiety of formulae A1:

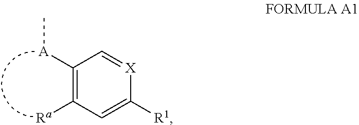

wherein

A and R¹ are the same as in FORMULA 1.

X is selected from CR′″ and N, wherein

R″′ and is selected from hydrogen, halogen, hydroxyl, amino, cyano, nitro, optionally substituted C₁-C₆ alkyl, optionally substituted C₂-C₆ alkenyl, optionally substituted C₂-C₆ alkynyl, optionally substituted C₁-C₆ alkoxy, optionally substituted C₁-C₆ alkylamino, optionally substituted 3-6 membered cycloalkyl, optionally substituted 3-6 membered cycloalkoxy, optionally substituted 3-6 membered cycloalkylamino, optionally substituted 4-6 membered heterocyclyl, optionally substituted aryl, and optionally substituted heteroaryl; and

R^{a} is optionally formed a ring with A, and is selected from null, hydrogen, halogen, R^{b}NR¹⁶, R^{b}OR¹⁶, R^{b}SR¹⁶, R^{b}NR¹⁶R¹⁷, R^{b}OCOR¹⁶, R^{b}OCO₂R¹⁶, R^{b}OCONR¹⁶R¹⁷, R^{b}COR¹⁶, R^{b}CO₂R¹⁶, R^{b}CONR¹⁶R¹⁷, R^{b}SOR¹⁶, R^{b}SO₂R¹⁶, R^{b}SO₂NR¹⁶R¹⁷, R^{b}NR¹⁸CO₂R¹⁶, R^{b}NR¹⁸COR¹⁶, R^{b}NR¹⁸C(O)NR¹⁶R¹⁷, R^{b}NR¹⁸SOR¹⁶, R^{b}NR¹⁸SO₂R¹⁶, R^{b}NR¹⁸SO₂NR¹⁶R¹⁷, optionally substituted C₁-C₈ alkyl, optionally substituted C₁-C₈ alkylene, optionally substituted C₂-C₈ alkenyl, optionally substituted C₂-C₈ alkenylene, optionally substituted C₂-C₈ alkynyl, optionally substituted C₂-C₈ alkynylene, optionally substituted 3-10 membered carbocyclyl, optionally substituted 4-10 membered heterocyclyl, optionally substituted aryl, and optionally substituted heteroaryl, wherein

R^{b} is null, or a bivalent or trivalent moiety selected from optionally substituted C₁-C₈ alkylene, optionally substituted C₂-C₈ alkenylene, optionally substituted C₂-C₈ alkynylene, optionally substituted C₁-C₈alkoxyC₁-C₈alkylene, optionally substituted C₁-C₈alkylaminoC₁-C₈alkylene, optionally substituted C₁-C₈ haloalkylene, optionally substituted C₁-C₈ hydroxyalkylene, optionally substituted 3-10 membered carbocyclyl, optionally substituted 4-10 membered heterocyclyl, optionally substituted aryl, and optionally substituted heteroaryl;

R¹⁶, R¹⁷, and R¹⁸ are independently selected from null, hydrogen, optionally substituted C₁-C₈ alkyl, optionally substituted C₂-C₈ alkenyl, optionally substituted C₂-C₈ alkynyl, optionally substituted C₁-C₈alkoxyC₁-C₈alkyl, optionally substituted C₁-C₈alkylaminoC₁-C₈alkyl, optionally substituted 3-10 membered carbocyclyl, optionally substituted 4-10 membered heterocyclyl, optionally substituted aryl, and optionally substituted heteroaryl, or

R¹⁶ and R¹⁷, R¹⁶ and R¹⁸ together with the atom to which they are connected form a 3-20 membered cycloalkyl or heterocyclyl ring.

In another embodiment, A is null.

In another embodiment, A is null; Ar is a bicyclic aryl or a bicyclic heteroaryl; and A-Ar—R¹ is a moiety of FORMULAE A2 or A3:

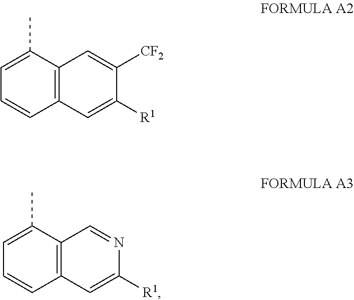

wherein

R¹ is the same as in FORMULA 1.

In another embodiment, A is NR⁴, wherein

R⁴ is selected from hydrogen, optionally substituted C₁-C₈ alkyl, optionally substituted C₂-C₈ alkenyl, optionally substituted C₂-C₈ alkynyl, optionally substituted C₁-C₈alkoxyC₁-C₈alkyl, optionally substituted C₁-C₈alkylaminoC₁-C₈alkyl, optionally substituted 3-10 membered carbocyclyl, optionally substituted 4-10 membered heterocyclyl, optionally substituted aryl, and optionally substituted heteroaryl.

In another embodiment, A is NR⁴; and A-Ar—R¹ is a moiety of FORMULAE A4, A5 or A6:

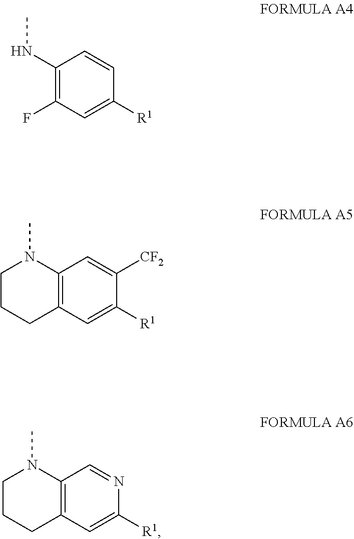

wherein

R¹ is the same as in FORMULA 1.

In another embodiment, R¹ is selected from optionally substituted 3-10 membered carbocyclyl, optionally substituted 4-10 membered heterocyclyl, optionally substituted aryl and optionally substituted heteroaryl.

In another embodiment, R¹ is selected from optionally substituted aryl and optionally substituted heteroaryl.

In another embodiment, R¹ is selected from optionally substituted pyrazole and optionally substituted pyridinyl.

In another embodiment, R² is selected from optionally substituted C₁-C₈ alkylene, optionally substituted 3-10 membered carbocyclyl, optionally substituted 4-8 membered heterocycly, optionally substituted aryl, and optionally substituted heteroaryl.

In another embodiment, R³ is selected from COR¹⁴ and CONR¹⁴R¹⁵.

In another embodiment, R³ is selected from COMe and CONHMe.

In another embodiment, the CBP/P300 ligand comprises a moiety of FORMULA 2:

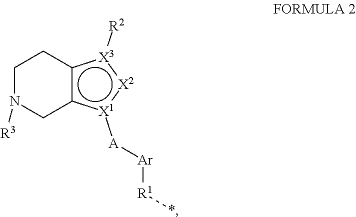

wherein

the linker moiety of the bivalent compound is attached to R¹; and

X¹ and X³ are independently selected from C and N, with the proviso that at least one of X¹ and X³ is C and at most only one of X¹ and X³ is N;

X² is selected from CR′, O, and NR′, wherein

R′ is selected from H, optionally substituted C₁-C₈ alkyl, and optionally substituted 3-10 membered carbocyclyl;

A is selected from null, CR⁴R⁵, CO, O, S, SO, SO₂, and NR⁴, wherein

R⁴ and R⁵ are independently selected from hydrogen, halogen, hydroxyl. amino, cyano, nitro, optionally substituted C₁-C₈ alkyl, optionally substituted C₂-C₈ alkenyl, optionally substituted C₂-C₈ alkynyl, optionally substituted C₁-C₈ alkoxy, optionally substituted C₁-C₈alkoxyC₁-C₈alkyl, optionally substituted C₁-C₈ alkylamino, optionally substituted C₁-C₈alkylaminoC₁-C₈alkyl, optionally substituted 3-10 membered carbocyclyl, optionally substituted 3-8 membered cycloalkoxy, optionally substituted 3-10 membered carbocyclylamino, optionally substituted 4-10 membered heterocyclyl, optionally substituted aryl, and optionally substituted heteroaryl;

Ar and R⁴, Ar and R⁵, and/or R⁴ and R⁵ together with the atom to which they are connected form an optionally substituted 3-20 membered cycloalkyl or 4-20 membered heterocyclyl ring;

Ar is selected from aryl, heteroaryl, bicyclic aryl, bicyclic heteroaryl, tricyclic aryl, tricyclic heteroaryl groups, each of which is substituted with R¹ and optionally substituted with one or more substituents independently selected from hydrogen, halogen, oxo, CN, NO₂, OR⁶, SR⁶, NR⁶R⁷, OCOR⁶, OCO₂R⁶, OCONR⁶R⁷, COR⁶, CO₂R⁶, CONR⁶R⁷, SOR⁶, SO₂R⁶, SO₂NR⁶R⁷, NR⁸CO₂R⁶, NR⁸COR⁶, NR⁸C(O)NR⁶R⁷, NR⁸SOR⁶, NR⁸SO₂R⁶, NR⁸SO₂NR⁶R⁷, optionally substituted C₁-C₈ alkyl, optionally substituted C₂-C₈ alkenyl, optionally substituted C₂-C₈ alkynyl, optionally substituted C₁-C₈ alkoxy, optionally substituted C₁-C₈alkoxyC₁-C₈alkyl, optionally substituted C₁-C₈alkylamino, optionally substituted C₁-C₈alkylaminoC₁-C₈alkyl, optionally substituted 3-10 membered carbocyclyl, optionally substituted 3-8 membered cycloalkoxy, optionally substituted 3-10 membered carbocyclylamino, optionally substituted 4-10 membered heterocyclyl, optionally substituted aryl, and optionally substituted heteroaryl, wherein

R⁶, R⁷, and R⁸ are independently selected from hydrogen, optionally substituted C₁-C₈ alkyl, optionally substituted C₂-C₈ alkenyl, optionally substituted C₂-C₈ alkynyl, optionally substituted C₁-C₈alkoxyC₁-C₈alkyl, optionally substituted C₁-C₈alkylaminoC₁-C₈alkyl, optionally substituted 3-10 membered carbocyclyl, optionally substituted 4-10 membered heterocyclyl, optionally substituted aryl, and optionally substituted heteroaryl, or

R⁶ and R⁷, R⁶ and R⁸ together with the atom to which they are connected form a 4-20 membered heterocyclyl ring; and

R¹ is connected to the “linker” moiety of the bivalent compound, and R¹ is selected from null, R″O, R″S, R″NR⁹, R″OC(O), R″OC(O)O, R″OCONR⁹, R″C(O), R″C(O)O, R″CONR⁹, R″S(O), R″S(O)₂, R″SO₂NR⁹, R″NR¹⁰C(O)O, R″NR¹⁰C(O), R″NR¹⁰C(O)NR⁹, R″NR¹⁰S(O), R″NR¹⁰S(O)₂, R″NR¹⁰S(O)₂NR⁹, optionally substituted C₁-C₈ alkylene, optionally substituted C₂-C₈ alkenylene, optionally substituted C₂-C₈ alkynylene, optionally substituted 3-10 membered carbocyclyl, optionally substituted 4-8 membered heterocycly, optionally substituted aryl, and optionally substituted heteroaryl, wherein

R″ is null, or a bivalent moiety selected from optionally substituted C₁-C₈ alkylene, optionally substituted C₂-C₈ alkenylene, optionally substituted C₂-C₈ alkynylene, optionally substituted C₁-C₈alkoxyC₁-C₈alkylene, optionally substituted C₁-C₈ haloalkylene, optionally substituted C₁-C₈ hydroxyalkylene, optionally substituted 3-10 membered carbocyclyl, optionally substituted 4-10 membered heterocyclyl, optionally substituted C₃-C₁₃ fused cycloalkyl, optionally substituted C₃-C₁₃ fused heterocyclyl, optionally substituted C₃-C₁₃ bridged cycloalkyl, optionally substituted C₃-C₁₃ bridged heterocyclyl, optionally substituted C₃-C₁₃ spiro cycloalkyl, optionally substituted C₃-C₁₃ spiro heterocyclyl, optionally substituted aryl, and optionally substituted heteroaryl;

R⁹ and R¹⁰ are independently selected from optionally substituted C₁-C₈ alkyl, optionally substituted C₂-C₈ alkenyl, optionally substituted C₂-C₈ alkynyl, optionally substituted C₁-C₈alkoxyC₁-C₈alkyl, optionally substituted C₁-C₈alkylaminoC₁-C₈alkyl, optionally substituted 3-10 membered carbocyclyl, optionally substituted 4-10 membered heterocyclyl, optionally substituted aryl, and optionally substituted heteroaryl, or

R⁹ and R¹⁰ together with the atom to which they are connected form a 3-20 membered cycloalkyl or 4-20 membered heterocyclyl ring; and

R² is selected from hydrogen, halogen, CN, NO₂, OR^{B}, SR¹¹, NR¹¹R¹², OCOR¹¹, OCO₂R¹¹, OCONR¹¹R¹², COR¹¹, CO₂R¹¹, CONR¹¹R¹², SOR¹¹, SO₂R¹¹, SO₂NR¹¹R¹², NR¹³CO₂R¹¹, NR¹³COR¹¹, NR¹³C(O)NR¹¹R¹², NR¹³SOR¹¹, NR¹³SO₂R¹¹, NR¹³SO₂NR¹¹R¹², optionally substituted C₁-C₈ alkyl, optionally substituted C₂-C₈ alkenyl, optionally substituted C₂-C₈ alkynyl, optionally substituted C₁-C₈ alkoxy, optionally substituted C₁-C₈alkoxyC₁-C₈alkyl, optionally substituted C₁-C₈alkylamino, optionally substituted C₁-C₈alkylaminoC₁-C₈alkyl, optionally substituted 3-10 membered carbocyclyl, optionally substituted 3-8 membered cycloalkoxy, optionally substituted 3-10 membered carbocyclylamino, optionally substituted 4-10 membered heterocyclyl, optionally substituted aryl, and optionally substituted heteroaryl, wherein

R¹¹, R¹², and R¹³ are independently selected from hydrogen, optionally substituted C₁-C₈ alkyl, optionally substituted C₂-C₈ alkenyl, optionally substituted C₂-C₈ alkynyl, optionally substituted C₁-C₈alkoxyC₁-C₈alkyl, optionally substituted C₁-C₈alkylaminoC₁-C₈alkyl, optionally substituted 3-10 membered carbocyclyl, optionally substituted 4-10 membered heterocyclyl, optionally substituted aryl, and optionally substituted heteroaryl, or

R¹¹ and R¹², R¹¹ and R¹³ together with the atom to which they are connected form a 3-20 membered cycloalkyl or 4-20 membered heterocyclyl ring; and R³ is selected from hydrogen, COR¹⁴, CO₂R¹⁴, CONR¹⁴R¹⁵, SOR¹⁴, SO₂R¹⁴, SO₂NR¹⁴R¹⁵, optionally substituted C₁-C₆ alkyl, optionally substituted C₂-C₆ alkenyl, optionally substituted C₂-C₆ alkynyl, optionally substituted 3-6 membered cycloalkyl, optionally substituted 4-6 membered heterocyclyl, wherein

R¹⁴ and R¹⁵ are independently selected from hydrogen, optionally substituted C₁-C₆ alkyl, optionally substituted C₂-C₆ alkenyl, optionally substituted C₂-C₆ alkynyl, optionally substituted 3-6 membered cycloalkyl, and optionally substituted 4-6 membered heterocyclyl, or

R¹⁴ and R¹⁵ together with the atom to which they are connected form a 4-6 membered heterocyclyl ring.

In another embodiment, X¹ is C; and X² and X³ are N. The FORMULA 2 is FORMULA 2A:

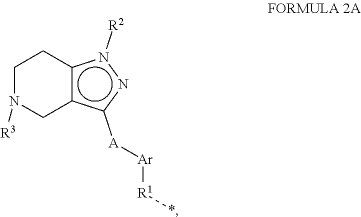

wherein

A, Ar, R¹, R² and R³ are the same as in FORMULA 2.

In another embodiment, A-Ar—R¹ is a moiety of formulae B1:

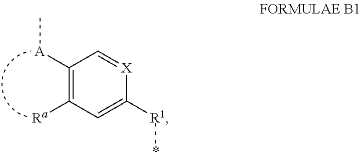

wherein

* indicates the connection to the linker moiety of the bivalent compound;

A and R¹ are the same as in FORMULA 2;

X is selected from CR′″ and N, wherein

R″′ and is selected from hydrogen, halogen, hydroxyl, amino, cyano, nitro, optionally substituted C₁-C₆ alkyl, optionally substituted C₂-C₆ alkenyl, optionally substituted C₂-C₆ alkynyl, optionally substituted C₁-C₆ alkoxy, optionally substituted C₁-C₆ alkylamino, optionally substituted 3-6 membered cycloalkyl, optionally substituted 3-6 membered cycloalkyl, optionally substituted 3-6 membered cycloalkoxy, optionally substituted 3-6 membered cycloalkylamino, optionally substituted 4-6 membered heterocyclyl; and

R^{a} optionally forms a ring with A, and is selected from null, hydrogen, halogen, R^{b}NR¹⁶, R^{b}OR¹⁶, R^{b}SR¹⁶, R^{b}NR¹⁶R¹⁷, R^{b}OCOR¹⁶, R^{b}OCO₂R¹⁶, R^{b}OCONR¹⁶R¹⁷, R^{b}COR¹⁶, R^{b}CO₂R¹⁶, R^{b}CONR¹⁶R¹⁷, R^{b}SOR¹⁶, R^{b}SO₂R¹⁶, R^{b}SO₂NR¹⁶R¹⁷, R^{b}NR¹⁸CO₂R¹⁶, R^{b}NR¹⁸COR¹⁶, R^{b}NR¹⁸C(O)NR¹⁶R¹⁷, R^{b}NR¹⁸SOR¹⁶, R^{b}NR¹⁸SO₂R¹⁶, R^{b}NR¹⁸SO₂NR¹⁶R¹⁷, optionally substituted C₁-C₈ alkyl, optionally substituted C₁-C₈ alkylene, optionally substituted C₂-C₈ alkenyl, optionally substituted C₂-C₈ alkenylene, optionally substituted C₂-C₈ alkynyl, optionally substituted C₂-C₈ alkynylene, optionally substituted 3-10 membered carbocyclyl, optionally substituted 4-10 membered heterocyclyl, optionally substituted aryl, and optionally substituted heteroaryl, wherein

R^{b} is null, or a bivalent or trivalent moiety selected from optionally substituted C₁-C₈ alkylene, optionally substituted C₂-C₈ alkenylene, optionally substituted C₂-C₈ alkynylene, optionally substituted C₁-C₈alkoxyC₁-C₈alkylene, optionally substituted C₁-C₈alkylaminoC₁-C₈alkylene, optionally substituted C₁-C₈ haloalkylene, optionally substituted C₁-C₈ hydroxyalkylene, optionally substituted 3-10 membered carbocyclyl, optionally substituted 4-10 membered heterocyclyl, optionally substituted aryl, and optionally substituted heteroaryl;

R¹⁶, R¹⁷, and R¹⁸ are independently selected from null, a bond, hydrogen, optionally substituted C₁-C₈ alkyl, optionally substituted C₂-C₈ alkenyl, optionally substituted C₂-C₈ alkynyl, optionally substituted C₁-C₈alkoxyC₁-C₈alkyl, optionally substituted C₁-C₈alkylaminoC₁-C₈alkyl, optionally substituted 3-10 membered carbocyclyl, optionally substituted 4-10 membered heterocyclyl, optionally substituted aryl, and optionally substituted heteroaryl, or

R¹⁶ and R¹⁷, R¹⁶ and R¹⁸ together with the atom to which they are connected form a 3-20 membered cycloalkyl or 4-20 membered heterocyclyl ring.

In another embodiment, A is null.

In another embodiment, A is null; Ar is a bicyclic aryl or a bicyclic heteroaryl; and A-Ar—R¹ is a moiety of FORMULAE B2 or B3:

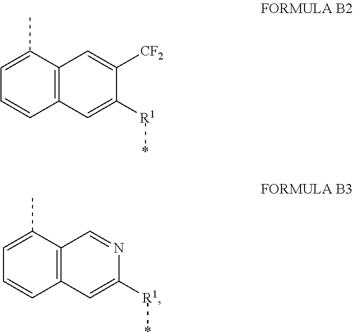

wherein

* indicates the connection to the linker moiety of the bivalent compound; and

R¹ is the same as in FORMULA 2.

In another embodiment, A is NR⁴, wherein

R⁴ is selected from hydrogen, optionally substituted C₁-C₈ alkyl, optionally substituted C₂-C₈ alkenyl, optionally substituted C₂-C₈ alkynyl, optionally substituted C₁-C₈alkoxyC₁-C₈alkyl, optionally substituted C₁-C₈alkylaminoC₁-C₈alkyl, optionally substituted 3-10 membered carbocyclyl, optionally substituted 4-10 membered heterocyclyl, optionally substituted aryl, and optionally substituted heteroaryl.

In another embodiment, A is NR⁴; and A-Ar—R¹ is a moiety of FORMULAE B4, B5 or B6:

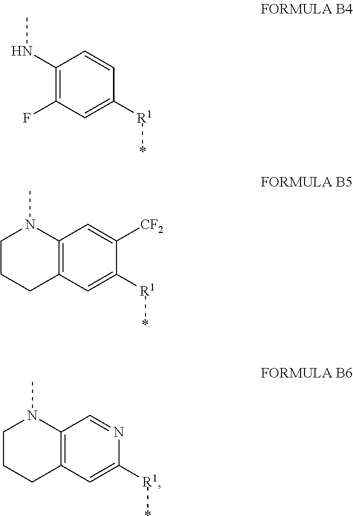

wherein

* indicates the connection to the linker moiety of the bivalent compound; and

R¹ is the same as in FORMULA 2.

In another embodiment, R¹ is selected from optionally substituted 3-10 membered carbocyclylene, optionally substituted 4-10 membered heterocyclyl, optionally substituted aryl, and optionally substituted heteroaryl.

In another embodiment, R¹ is selected from optionally substituted aryl and optionally substituted heteroaryl.

In another embodiment, R¹ is selected from optionally substituted pyrazole and optionally substituted pyridinyl.

In another embodiment, R² is selected from optionally substituted C₁-C₈ alkyl, optionally substituted 3-10 membered carbocyclyl, optionally substituted 4-10 membered heterocyclyl, optionally substituted aryl, and optionally substituted heteroaryl.

In another embodiment, R³ is selected from COR¹⁴ and CONR¹⁴R¹⁵.

In another embodiment, R³ is selected from COMe and CONHMe.

In another embodiment, the CBP/P300 ligand is derived from any of the following:

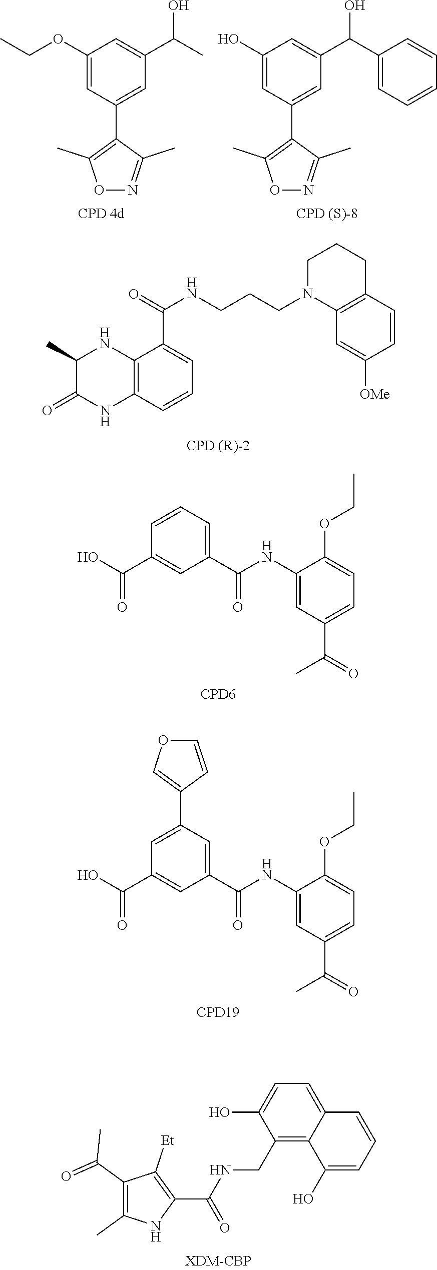

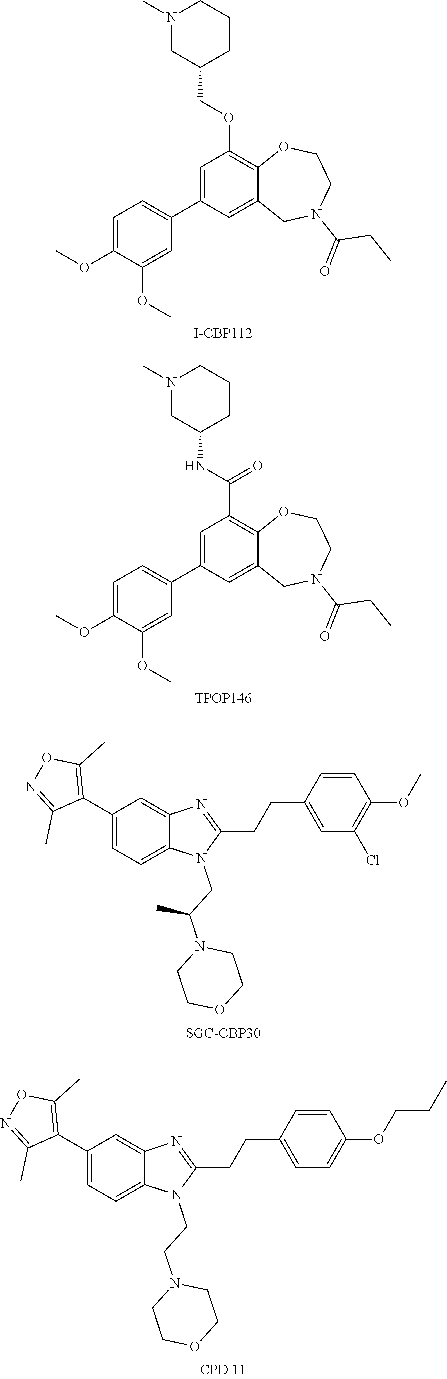

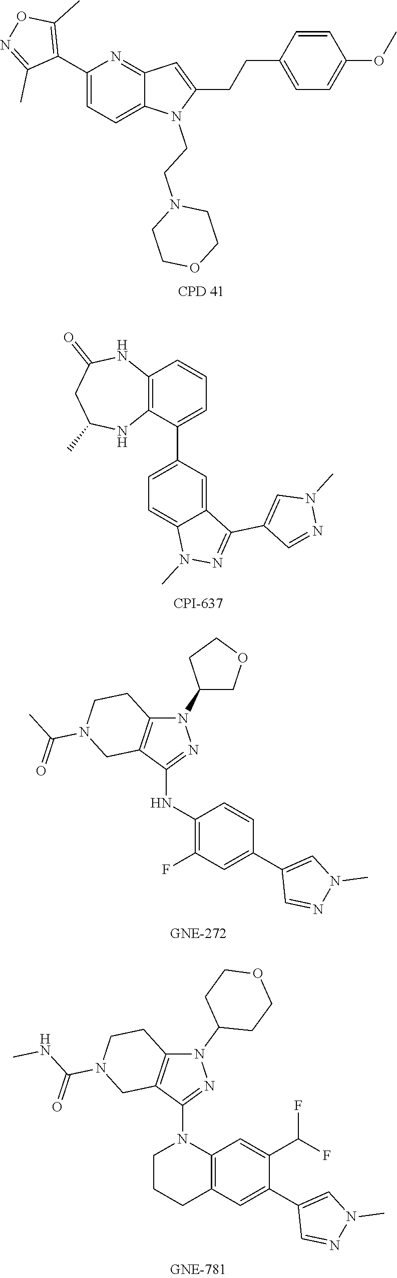

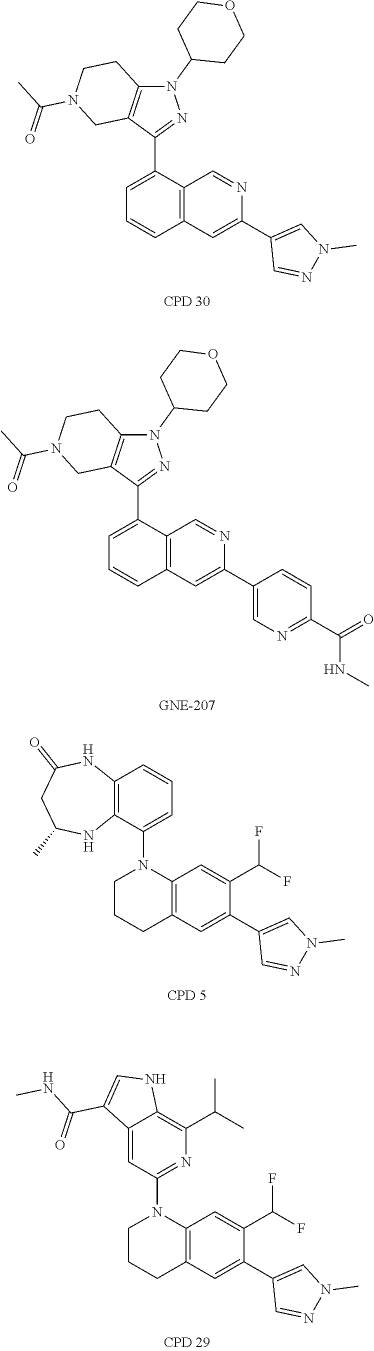

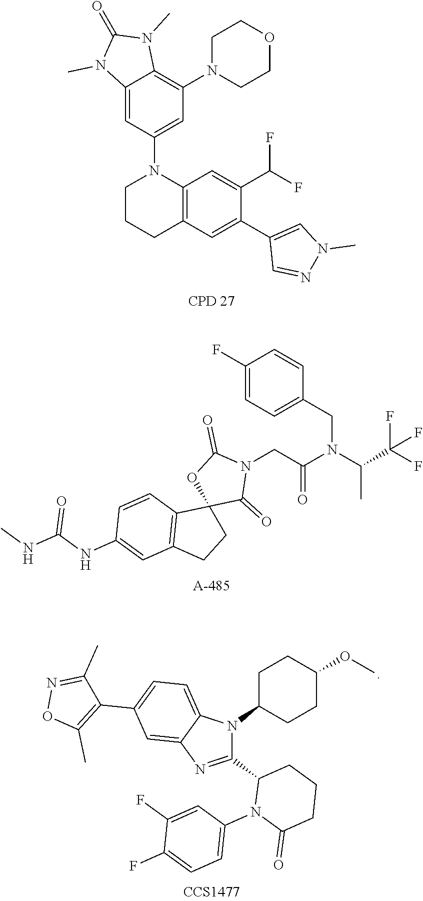

In another embodiment, the CBP/P300 ligand is derived from the following CBP/P300 inhibitors: C646, naphthol-AS-E, compound 1-10, MYBMIM, CCS1477, ICG-001, YH249, YH250, HBS1, OHM1, and KCN1.

In another embodiment, the CBP/P300 ligand is selected from the group consisting of:

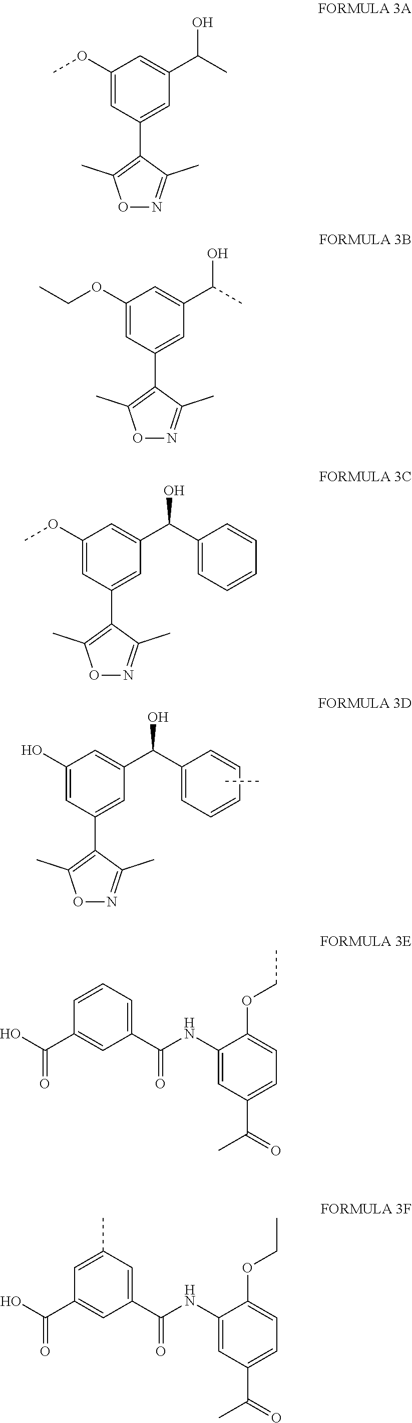

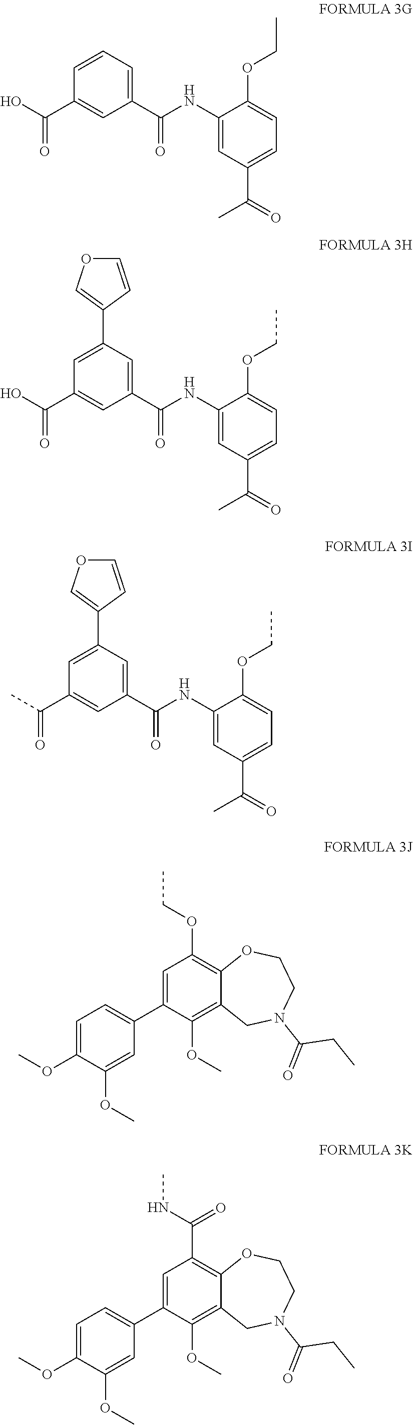

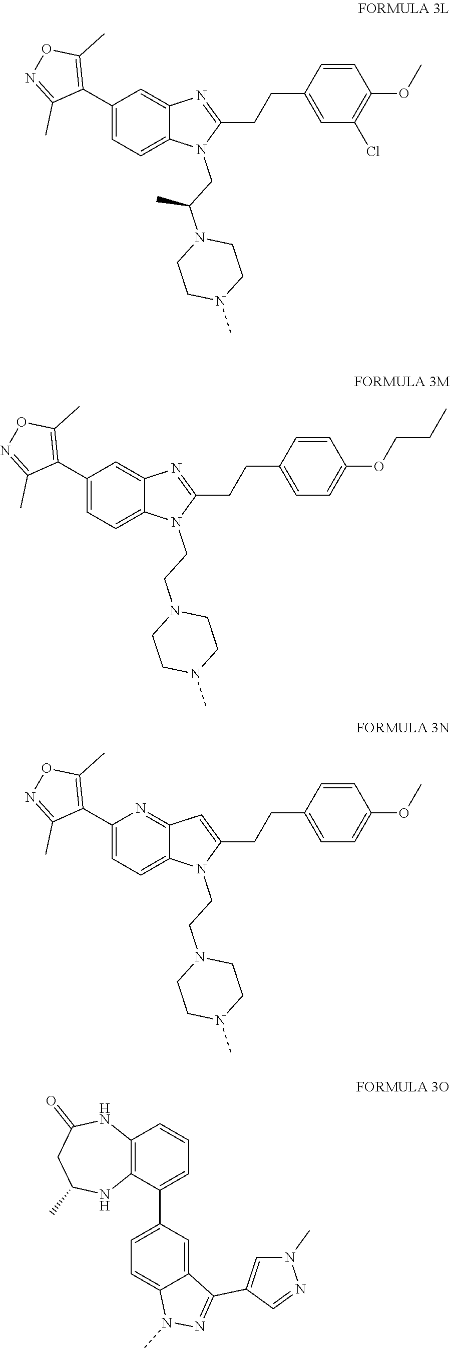

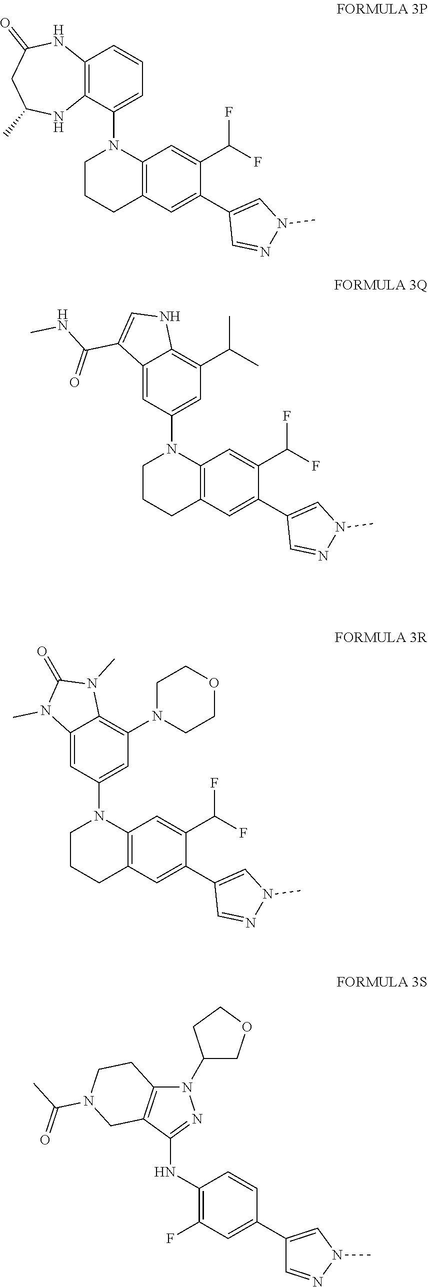

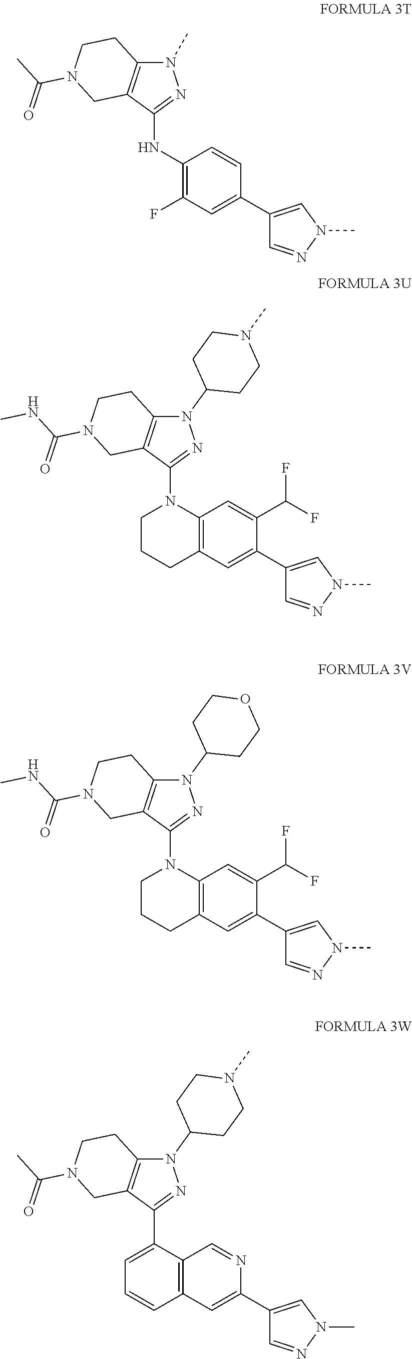

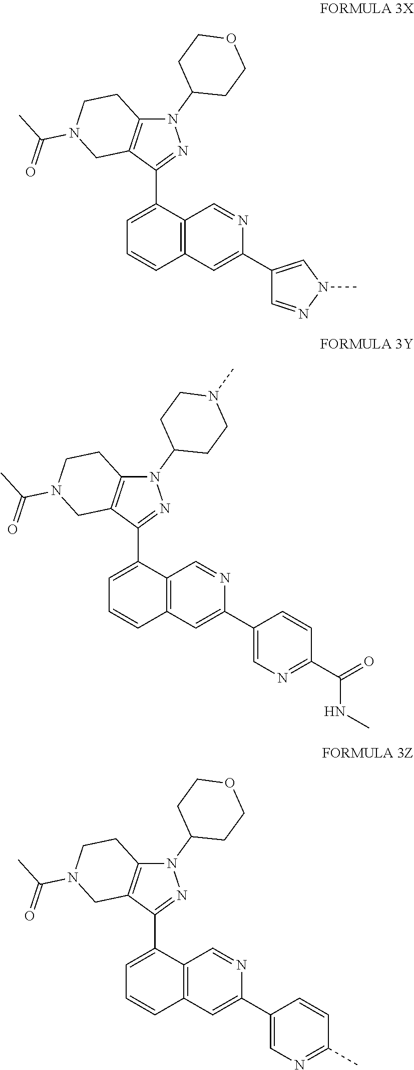

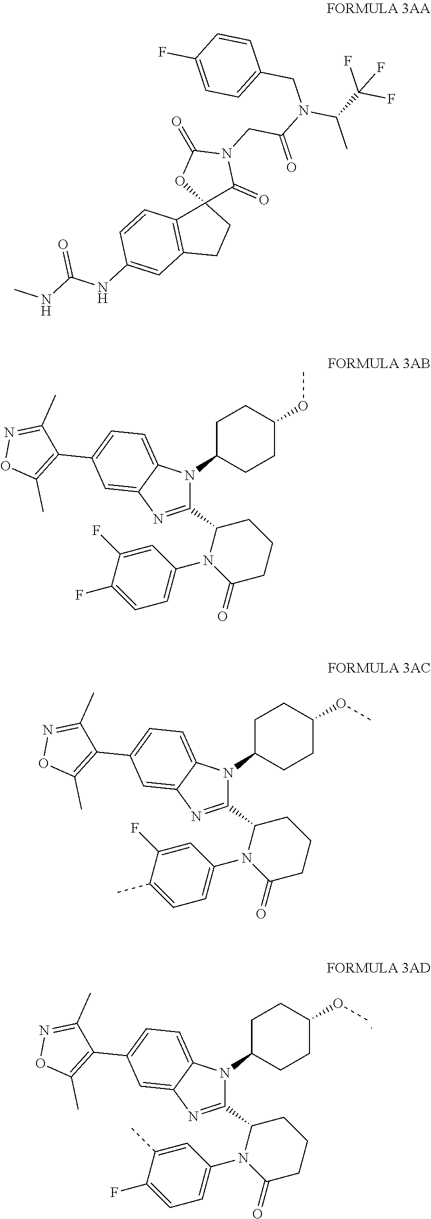

Degradation Tag

As used herein, the term “degradation tag” refers to a compound, which associates with or binds to an ubiquitin ligase for recruitment of the corresponding ubiquitination machinery to CBP/P300 or is a hydrophobic group or a tag that leads to misfolding of the CBP/P300 protein and subsequent degradation at the proteasome or loss of function.

In some embodiments, the degradation tag is a moiety selected from the group consisting of FORMULAE 5A, 5B, 5C, and 5D:

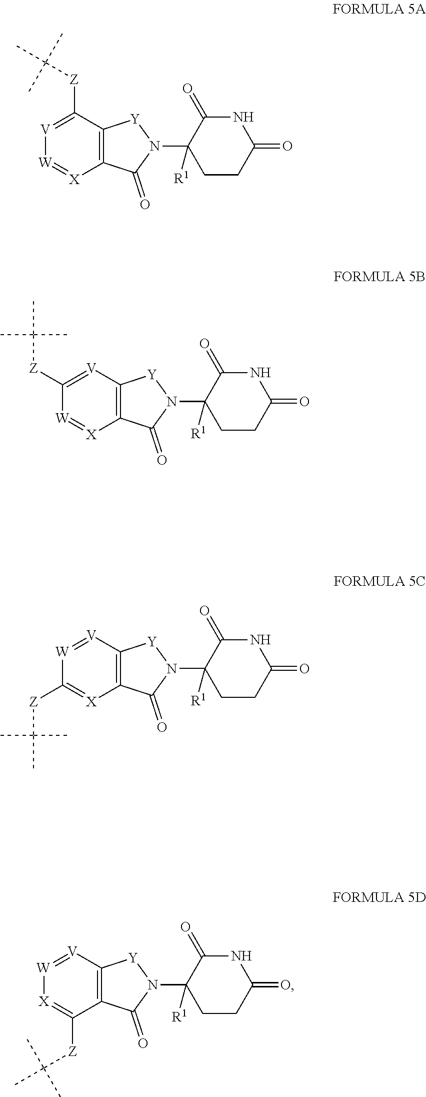

wherein

V, W, and X are independently selected from CR² and N;

Y is selected from CO, CR³R⁴, N═CR³, and N═N;

Z is selected from null, CO, CR⁵R⁶, NR⁵, O, optionally substituted C₁-C₁₀ alkylene, optionally substituted C₁-C₁₀ alkenylene, optionally substituted C₁-C₁₀ alkynylene, optionally substituted 3-10 membered carbocyclyl, optionally substituted 4-10 membered heterocyclyl, optionally substituted C₃-C₁₃ fused cycloalkyl, optionally substituted C₃-C₁₃ fused heterocyclyl, optionally substituted C₃-C₁₃ bridged cycloalkyl, optionally substituted C₃-C₁₃ bridged heterocyclyl, optionally substituted C₃-C₁₃ spiro cycloalkyl, optionally substituted C₃-C₁₃ spiro heterocyclyl, optionally substituted aryl, and optionally substituted heteroaryl; preferably, Z is selected from null, CH₂, CH═CH, C≡C, NH and O;

R¹, and R² are independently selected from hydrogen, halogen, cyano, nitro, optionally substituted C₁-C₆ alkyl, optionally substituted 3 to 6 membered carbocyclyl, and optionally substituted 4 to 6 membered heterocyclyl;

R³, and R⁴ are independently selected from hydrogen, halogen, cyano, nitro, optionally substituted C₁-C₆ alkyl, optionally substituted 3 to 6 membered carbocyclyl, and optionally substituted 4 to 6 membered heterocyclyl; or R³ and R⁴ together with the atom to which they are connected form a 3-6 membered carbocyclyl, or 4-6 membered heterocyclyl; and

R⁵ and R⁶ are independently selected from null, hydrogen, halogen, oxo, hydroxyl, amino, cyano, nitro, optionally substituted C₁-C₆ alkyl, optionally substituted 3 to 6 membered carbocyclyl, and optionally substituted 4 to 6 membered heterocyclyl; or R⁵ and R⁶ together with the atom to which they are connected form a 3-6 membered carbocyclyl, or 4-6 membered heterocyclyl.

In some embodiments, the degradation tag is a moiety selected from the group consisting of FORMULAE 5A, 5B, 5C, and 5D:

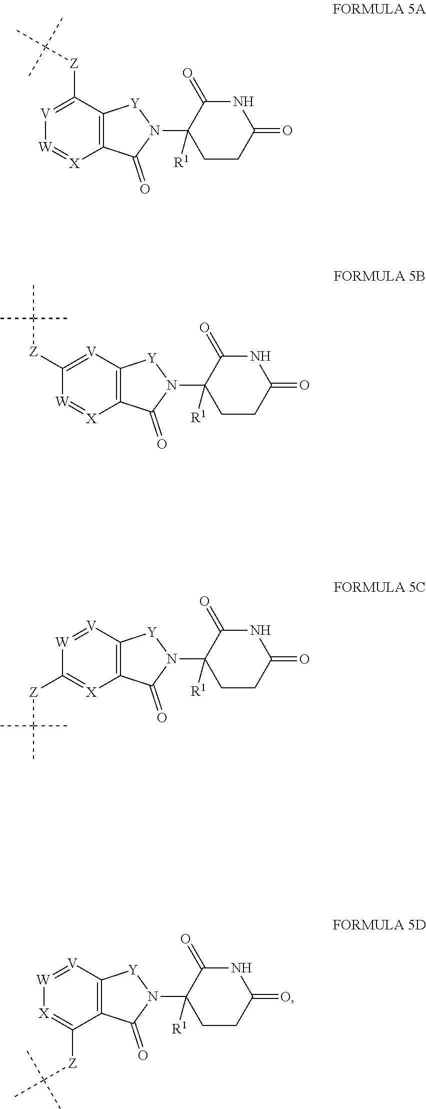

wherein

V, W, and X are independently selected from CR² and N;

Y is selected from CO, CH₂, and N═N;

Z is selected from CH₂, NH and O; and

R¹ and R² are independently selected from hydrogen, halogen, cyano, nitro, and C₁-C₅ alkyl.

In some embodiments, the degradation tag is a moiety selected from the group consisting of FORMULAE 5E, 5F, 5G, 5H, 5I, 5J, 5K, 5L, 5M, 5N, 5O, 5P, and 5Q:

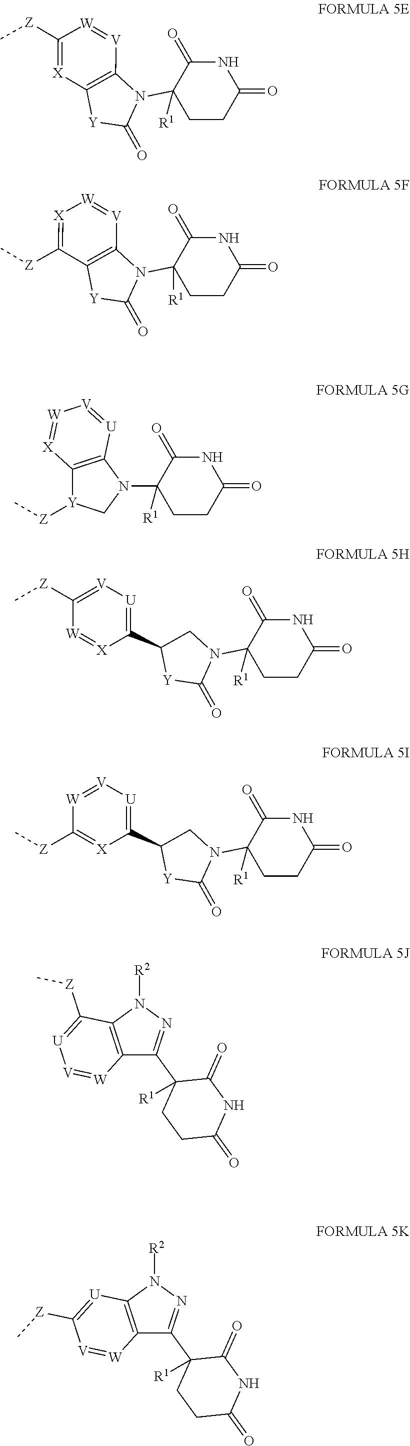

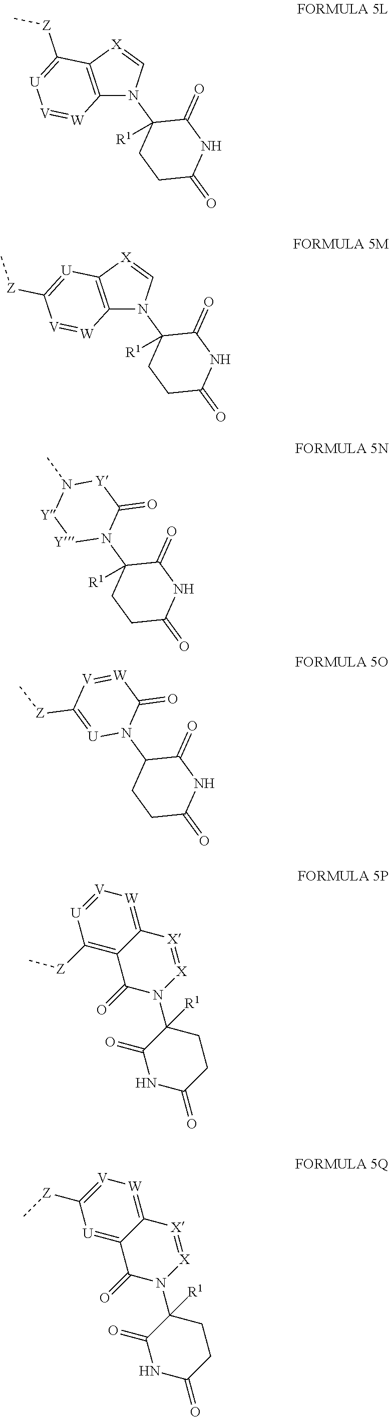

wherein

U, V, W, X and X′ are independently selected from CR² and N;

Y is selected from CR³R⁴, NR³ and 0; preferably, Y is selected from CH₂, NH, NCH₃ and O;

Y′, Y″, and Y′″ are independently selected from CR³R⁴;

Z is selected from null, CO, CR⁵R⁶, NR⁵, O, optionally substituted C₁-C₁₀ alkylene, optionally substituted C₁-C₁₀ alkenylene, optionally substituted C₁-C₁₀ alkynylene, optionally substituted 3-10 membered carbocyclyl, optionally substituted 4-10 membered heterocyclyl, optionally substituted C₃-C₁₃ fused cycloalkyl, optionally substituted C₃-C₁₃ fused heterocyclyl, optionally substituted C₃-C₁₃ bridged cycloalkyl, optionally substituted C₃-C₁₃ bridged heterocyclyl, optionally substituted C₃-C₁₃ spiro cycloalkyl, optionally substituted C₃-C₁₃ spiro heterocyclyl, optionally substituted aryl, and optionally substituted heteroaryl; preferably, Z is selected from null, CH₂, CH═CH, C≡C, NH and O;

R¹, and R² are independently selected from hydrogen, halogen, cyano, nitro, optionally substituted C₁-C₆ alkyl, optionally substituted 3 to 6 membered carbocyclyl, and optionally substituted 4 to 6 membered heterocyclyl;

R³, and R⁴ are independently selected from hydrogen, halogen, cyano, nitro, optionally substituted C₁-C₆ alkyl, optionally substituted 3 to 6 membered carbocyclyl, and optionally substituted 4 to 6 membered heterocyclyl; or R³ and R⁴ together with the atom to which they are connected form a 3-6 membered carbocyclyl, or 4-6 membered heterocyclyl; and

R⁵ and R⁶ are independently selected from null, hydrogen, halogen, oxo, hydroxyl, amino, cyano, nitro, optionally substituted C₁-C₆ alkyl, optionally substituted 3 to 6 membered carbocyclyl, and optionally substituted 4 to 6 membered heterocyclyl; or R⁵ and R⁶ together with the atom to which they are connected form a 3-6 membered carbocyclyl, or 4-6 membered heterocyclyl; and

R′ is selected from hydrogen, optionally substituted C₁-C₆ alkyl, optionally substituted 3 to 6 membered carbocyclyl, and optionally substituted 4 to 6 membered heterocyclyl.

In one embodiment, the degradation tag is a moiety of FORMULA 6A:

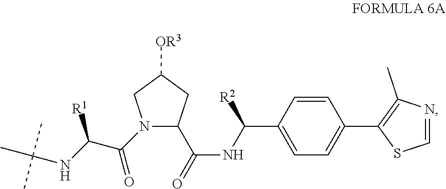

wherein

R¹ and R² are independently selected from hydrogen, hydroxyl, amino, cyano, nitro, optionally substituted C₁-C₈ alkyl, optionally substituted C₂-C₈ alkenyl, optionally substituted C₂-C₈ alkynyl; optionally substituted C₁-C₈alkoxyC₁-C₈alkyl, optionally substituted C₁-C₈ haloalkyl, optionally substituted C₁-C₈ hydroxyalkyl, optionally substituted C₁-C₈ aminoalkyl, optionally substituted C₁-C₈alkylaminoC₁-C₈alkyl, optionally substituted 3-10 membered carbocyclyl, and optionally substituted 4-10 membered heterocyclyl; and

R³ is selected from hydrogen, optionally substituted C(O)C₁-C₈ alkyl, optionally substituted C(O)C₁-C₈alkoxyC₁-C₈alkyl, optionally substituted C(O)C₁-C₈ haloalkyl, optionally substituted C(O)C₁-C₈ hydroxyalkyl, optionally substituted C(O)C₁-C₈ aminoalkyl, optionally substituted C(O)C₁-C₈alkylaminoC₁-C₈alkyl, optionally substituted C(O)(3-10 membered carbocyclyl), optionally substituted C(O)(4-10 membered heterocyclyl), optionally substituted C(O)C₂-C₈ alkenyl, optionally substituted C(O)C₂-C₈ alkynyl, optionally substituted C(O)OC₁-C₈alkoxyC₁-C₈alkyl, optionally substituted C(O)OC₁-C₈ haloalkyl, optionally substituted C(O)OC₁-C₈ hydroxyalkyl, optionally substituted C(O)OC₁-C₈ aminoalkyl, optionally substituted C(O)OC₁-C₈alkylaminoC₁-C₈alkyl, optionally substituted C(O)O(3-10 membered carbocyclyl), optionally substituted C(O)O(4-10 membered heterocyclyl), optionally substituted C(O)OC₂-C₈ alkenyl, optionally substituted C(O)OC₂-C₈ alkynyl, optionally substituted C(O)NC₁-C₈alkoxyC₁-C₈alkyl, optionally substituted C(O)NC₁-C₈ haloalkyl, optionally substituted C(O)NC₁-C₈ hydroxyalkyl, optionally substituted C(O)NC₁-C₈ aminoalkyl, optionally substituted C(O)NC₁-C₈alkylaminoC₁-C₈alkyl, optionally substituted C(O)N(3-10 membered carbocyclyl), optionally substituted C(O)N(4-10 membered heterocyclyl), optionally substituted C(O)NC₂-C₈ alkenyl, optionally substituted C(O)NC₂-C₈ alkynyl, optionally substituted P(O)(OH)₂, optionally substituted P(O)(OC₁-C₈ alkyl)₂, and optionally substituted P(O)(OC₁-C₈ aryl)₂.

In some embodiments, the degradation tag is a moiety of FORMULAE 6B, 6C, and 6D:

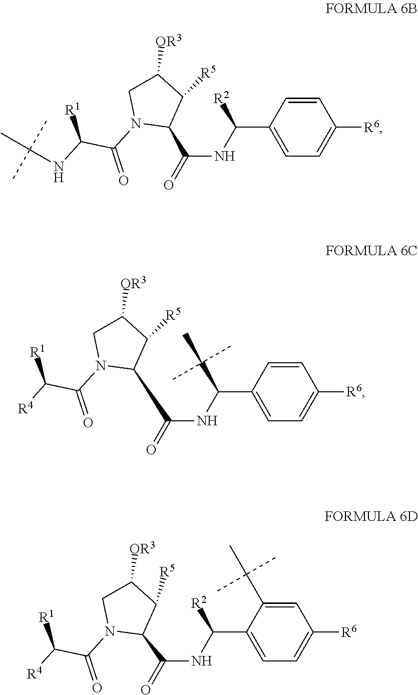

wherein

R¹ and R² are independently selected from hydrogen, hydroxyl, amino, cyano, nitro, optionally substituted C₁-C₈ alkyl, optionally substituted C₂-C₈ alkenyl, optionally substituted C₂-C₈ alkynyl; optionally substituted C₁-C₈alkoxyC₁-C₈alkyl, optionally substituted C₁-C₈ haloalkyl, optionally substituted C₁-C₈ hydroxyalkyl, optionally substituted C₁-C₈ aminoalkyl, optionally substituted C₁-C₈alkylaminoC₁-C₈alkyl, optionally substituted 3-10 membered carbocyclyl, and optionally substituted 4-10 membered heterocyclyl;

R³ is selected from hydrogen, optionally substituted C(O)C₁-C₈ alkyl, optionally substituted C(O)C₁-C₈alkoxyC₁-C₈alkyl, optionally substituted C(O)C₁-C₈ haloalkyl, optionally substituted C(O)C₁-C₈ hydroxyalkyl, optionally substituted C(O)C₁-C₈ aminoalkyl, optionally substituted C(O)C₁-C₈alkylaminoC₁-C₈alkyl, optionally substituted C(O)(3-10 membered carbocyclyl), optionally substituted C(O)(4-10 membered heterocyclyl), optionally substituted C(O)C₂-C₈ alkenyl, optionally substituted C(O)C₂-C₈ alkynyl, optionally substituted C(O)OC₁-C₈alkoxyC₁-C₈alkyl, optionally substituted C(O)OC₁-C₈ haloalkyl, optionally substituted C(O)OC₁-C₈ hydroxyalkyl, optionally substituted C(O)OC₁-C₈ aminoalkyl, optionally substituted C(O)OC₁-C₈alkylaminoC₁-C₈alkyl, optionally substituted C(O)O(3-10 membered carbocyclyl), optionally substituted C(O)O(4-10 membered heterocyclyl), optionally substituted C(O)OC₂-C₈ alkenyl, optionally substituted C(O)OC₂-C₈ alkynyl, optionally substituted C(O)NC₁-C₈alkoxyC₁-C₈alkyl, optionally substituted C(O)NC₁-C₈ haloalkyl, optionally substituted C(O)NC₁-C₈ hydroxyalkyl, optionally substituted C(O)NC₁-C₈ aminoalkyl, optionally substituted C(O)NC₁-C₈alkylaminoC₁-C₈alkyl, optionally substituted C(O)N(3-10 membered carbocyclyl), optionally substituted C(O)N(4-10 membered heterocyclyl), optionally substituted C(O)NC₂-C₈ alkenyl, optionally substituted C(O)NC₂-C₈ alkynyl, optionally substituted P(O)(OH)₂, optionally substituted P(O)(OC₁-C₈ alkyl)₂, and optionally substituted P(O)(OC₁-C₈ aryl)₂, and

R⁴ is selected from NR⁷R⁸,

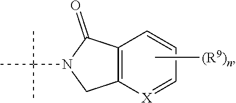

optionally substituted C₁-C₈alkoxy, optionally substituted 3-10 membered carbocyclyl, optionally substituted 4-10 membered heterocyclyl, optionally substituted aryl, and optionally substituted heteraryl, in which

R⁷ is selected from hydrogen, optionally substituted C₁-C₈alkyl, optionally substituted C₁-C₈cycloalkyl, optionally substituted C₁-C₈alkyl-CO, optionally substituted C₁-C₈cycloalkyl-CO, optionally substituted C₁-C₈cycloalkyl-C₁-C₈alkyl-CO, optionally substituted 4-10 membered heterocyclyl-CO, optionally substituted 4-10 membered heterocyclyl-C₁-C₈alkyl-CO, optionally substituted aryl-CO, optionally substituted aryl-C₁-C₈alkyl-CO, optionally substituted heteroaryl-CO, optionally substituted heteroaryl-C₁-C₈alkyl-CO, optionally substituted aryl, and optionally substituted heteroaryl;

R⁸ is selected from hydrogen, optionally substituted C₁-C₈alkyl, and optionally substituted C₁-C₈cycloalkyl;

R⁹, at each occurrence, is independently selected from hydrogen, halogen, cyano, optionally substituted C₁-C₈alkyl, optionally substituted C₁-C₈cycloalkyl, optionally substituted C₁-C₈heterocycloalkyl, optionally substituted C₁-C₈alkoxy, optionally substituted C₁-C₈cycloalkoxy, halo substituted C₁-C₈alkyl, halo substituted C₁-C₈cycloalkyl, halo substituted C₁-C₈alkoxl, halo substituted C₁-C₈cycloalkoxy, and halo substituted C₁-C₈heterocycloalkyl;

X is selected from CH and N; and

n is 0, 1, 2, 3, or 4;

R⁶ is selected from hydrogen, halogen, hydroxyl, amino, cyano, nitro, optionally substituted C₁-C₈alkyl, optionally substituted C₁-C₈cycloalkyl, optionally substituted C₁-C₈alkoxy, and optionally substituted C₁-C₈cycloalkoxy, optionally substituted C₁-C₈heterocycloalkyl, optionally substituted aryl, and optionally substituted heteroaryl, preferably, halogen, cyano, optionally substituted imidazole, optionally substituted pyrazole, optionally substituted oxadiazole, optionally substituted triazole, 4-methylthiazol-5-yl, or oxazol-5-yl group.

In another embodiment, the degradation tag is a moiety of FORMULA 7A:

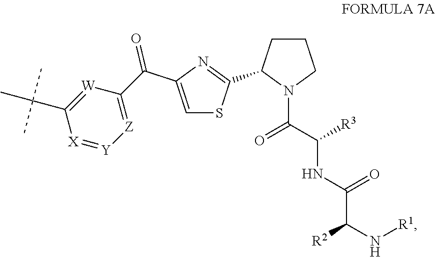

wherein

V, W, X, and Z are independently selected from CR⁴ and N; and

R¹, R², R³, and R⁴ are independently selected from hydrogen, halogen, cyano, nitro, optionally substituted C₁-C₈ alkyl, optionally substituted C₂-C₈ alkenyl, and optionally substituted C₂-C₈ alkynyl; optionally substituted C₁-C₈alkoxy, optionally substituted C₁-C₈alkylamino, optionally substituted C₁-C₈alkoxyC₁-C₈alkyl, optionally substituted C₁-C₈alkylaminoC₁-C₈alkyl, optionally substituted C₁-C₈ haloalkyl, optionally substituted C₁-C₈ hydroxyalkyl, optionally substituted 3-10 membered carbocyclyl, and optionally substituted 4-10 membered heterocyclyl.

In another embodiment, the degradation tag is a moiety of FORMULA 7B:

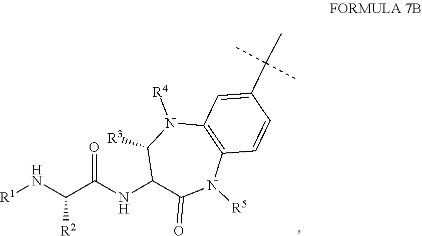

wherein

R¹, R², and R³ are independently selected from hydrogen, halogene, optionally substituted C₁-C₈ alkyl, optionally substituted C₁-C₈alkoxyC₁-C₈alkyl, optionally substituted C₁-C₈ haloalkyl, optionally substituted C₁-C₈ hydroxyalkyl, optionally substituted C₃-C₇ cycloalkyl, optionally substituted 3-7 membered heterocyclyl, optionally substituted C₂-C₈ alkenyl, and optionally substituted C₂-C₈ alkynyl;

R⁴ and R⁵ are independently selected from hydrogen, COR⁶, CO₂R⁶, CONR⁶R⁷, SOR⁶, SO₂R⁶, SO₂NR⁶R⁷, optionally substituted C₁-C₈ alkyl, optionally substituted C₁-C₈alkoxyC₁-C₈alkyl, optionally substituted C₁-C₈alkylaminoC₁-C₈alkyl, optionally substituted aryl-C₁-C₈alkyl, optionally substituted 3-8 membered cycloalkyl, optionally substituted 3-8 membered heterocyclyl, optionally substituted aryl, and optionally substituted heteroaryl, wherein

R⁶ and R⁷ are independently selected from hydrogen, optionally substituted C₁-C₈ alkyl, optionally substituted C₁-C₈alkoxyC₁-C₈alkyl, optionally substituted C₁-C₈alkylaminoC₁-C₈alkyl, optionally substituted 3-8 membered cycloalkyl, optionally substituted 3-8 membered heterocyclyl, optionally substituted aryl, and optionally substituted heteroaryl, or

R⁶ and R⁷ together with the atom to which they are connected form a 4-8 membered cycloalkyl or heterocyclyl ring.

In another embodiment, the degradation tag is a moiety of FORMULA 5A, 5B, 5C, 5D, 5E, 5F, 5G, 5H, and 5I.

In another embodiment, the degradation tag is a moiety of FORMULA 5A, 5B, 5C, 5D, 5E, and 5F.

In another embodiment, the degradation tag is derived from any of the following:

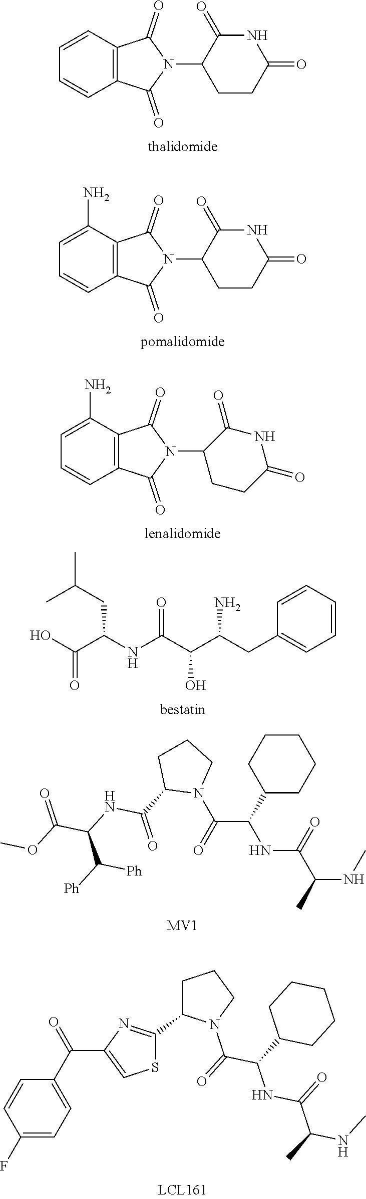

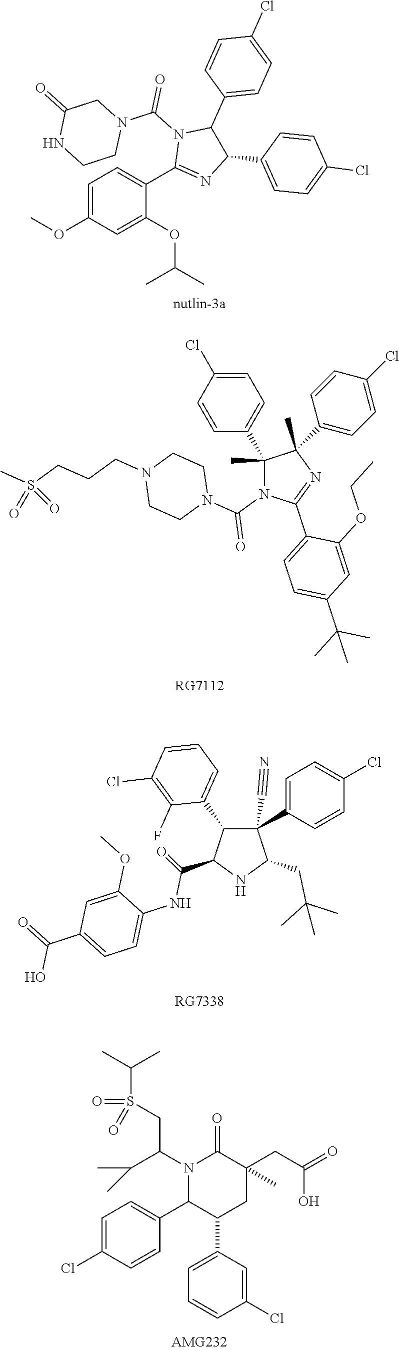

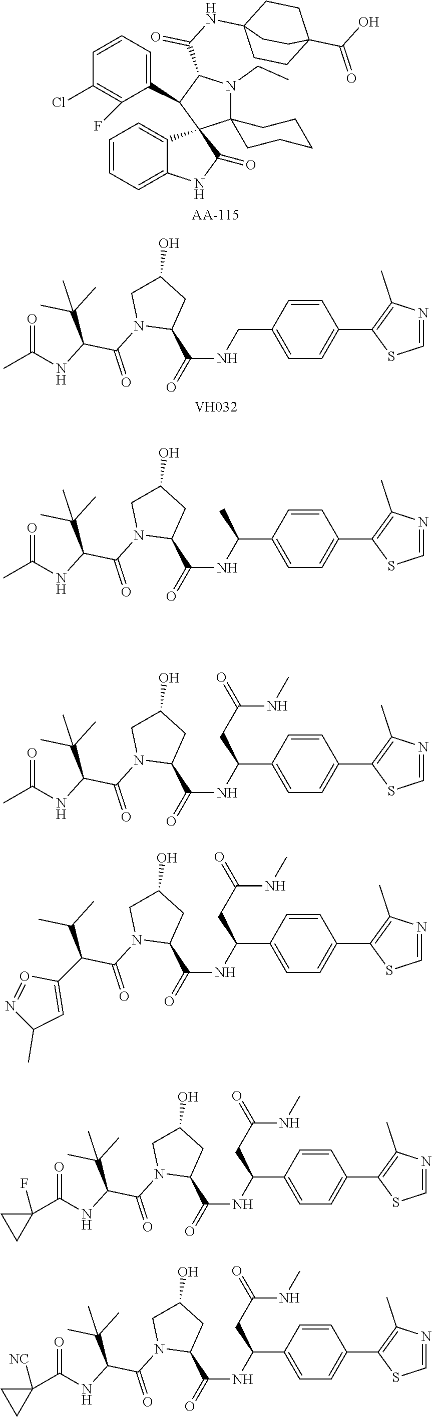

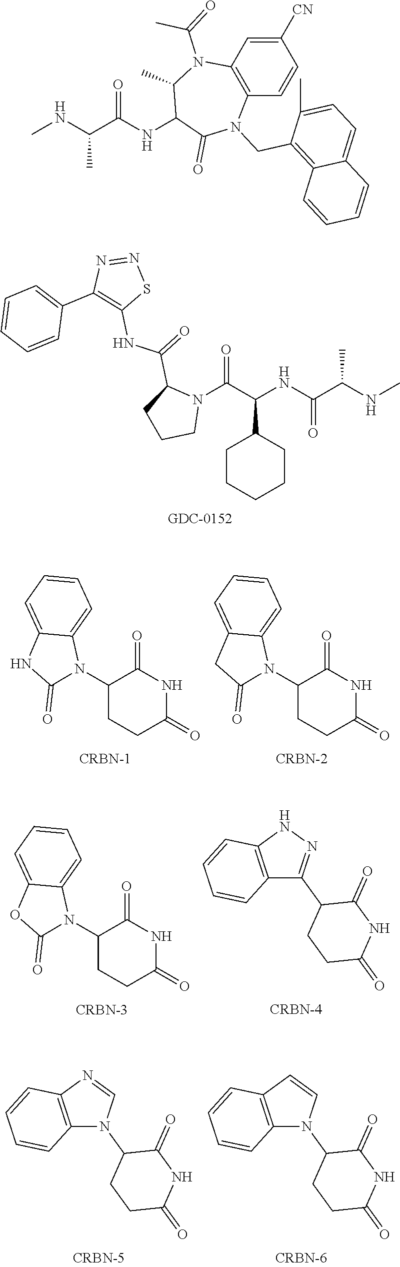

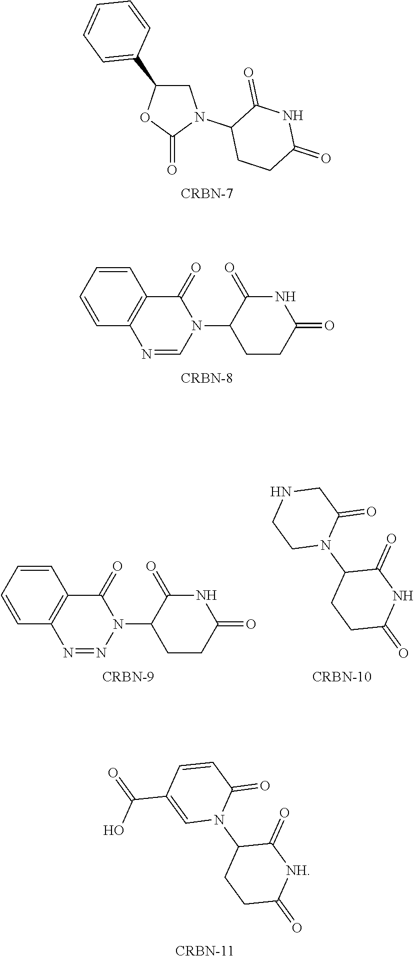 [SMILES: O=C1CCC(N2C[C@H](c3ccccc3)OC2=O)C(=O)N1 ; O=C1CCC(n2cnc3ccccc3c2=O)C(=O)N1 ; O=C1CCC(n2nnc3ccccc3c2=O)C(=O)N1 ; O=C1CCC(N2CCNCC2=O)C(=O)N1 ; O=C1CCC(n2cc(C(=O)O)ccc2=O)C(=O)N1]

In another embodiment, the degradation tag is derived from any of the following: thalidomide, pomalidomide, lenalidomide, CRBN-1, CRBN-2, CRBN-3, CRBN-4, CRBN-5, CRBN-6, CRBN-7, CRBN-8, CRBN-9, CRBN-10, and CRBN-11.

In another embodiment, the degradation tag is selected from the group consisting of:

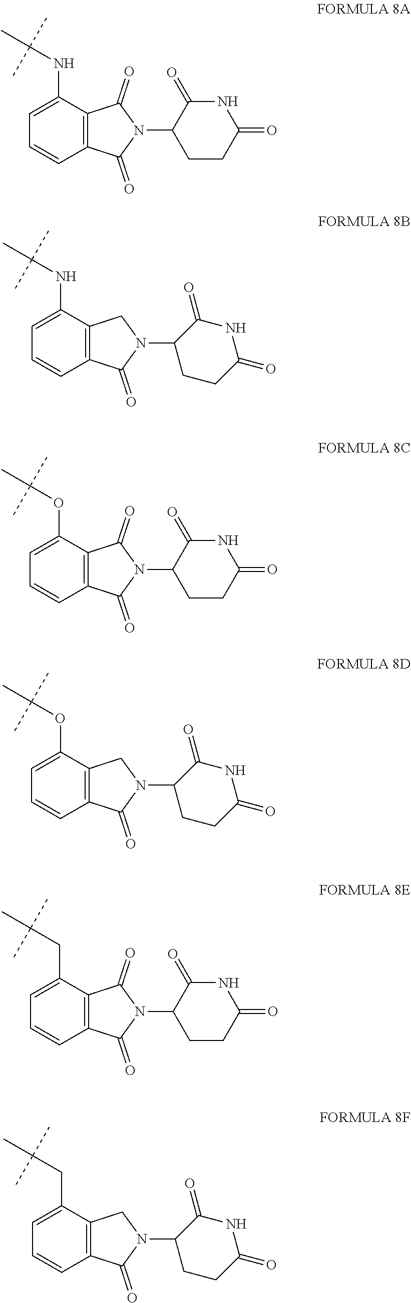

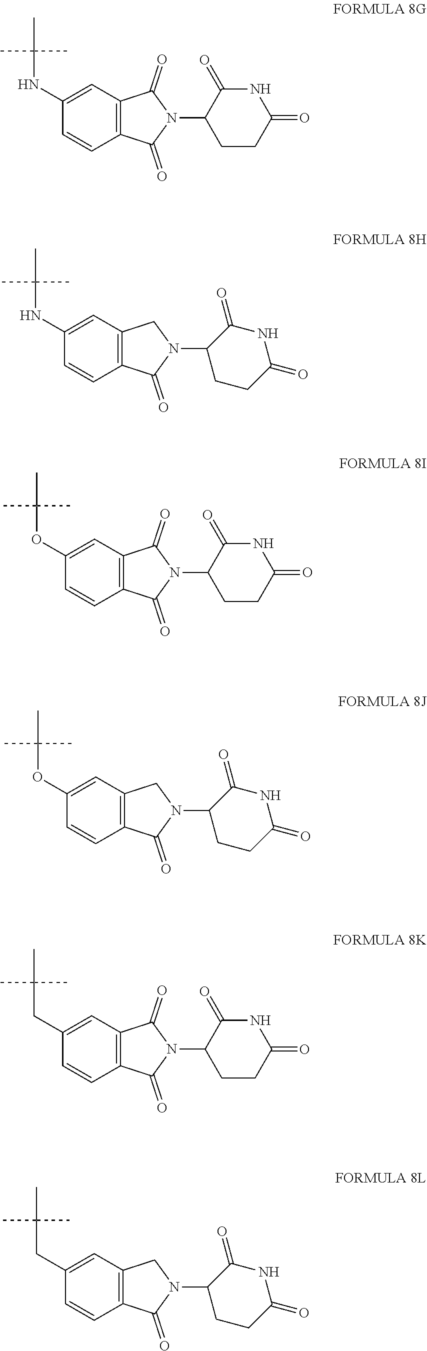

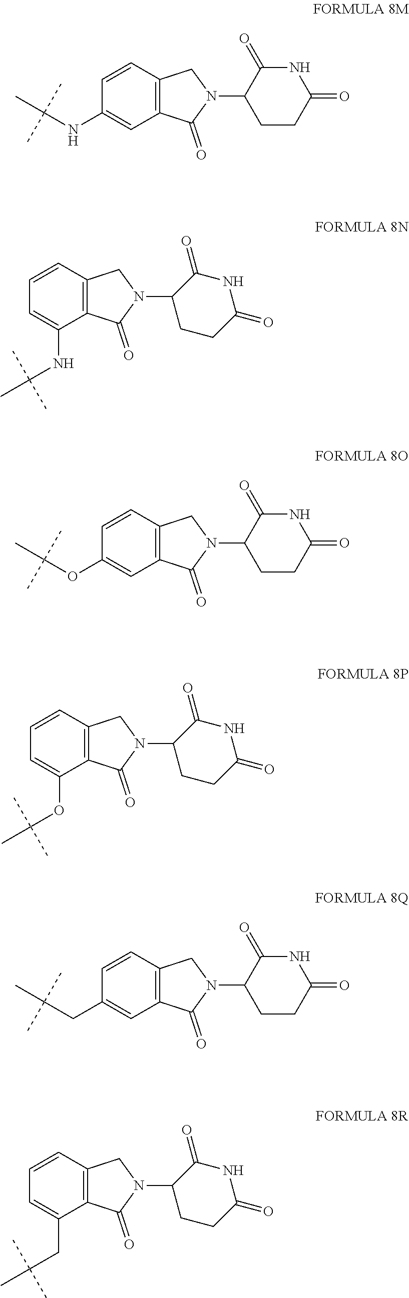

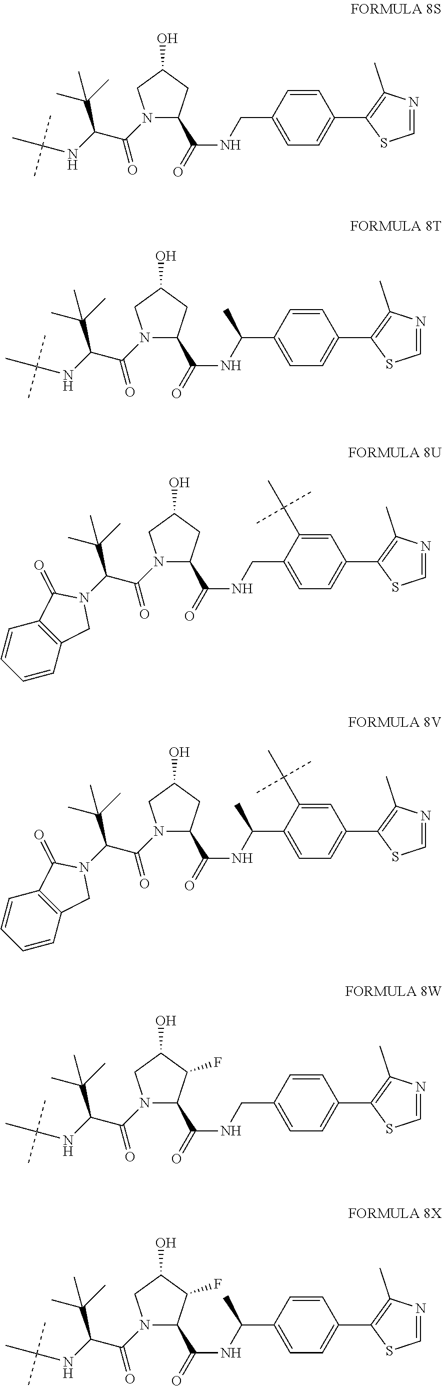

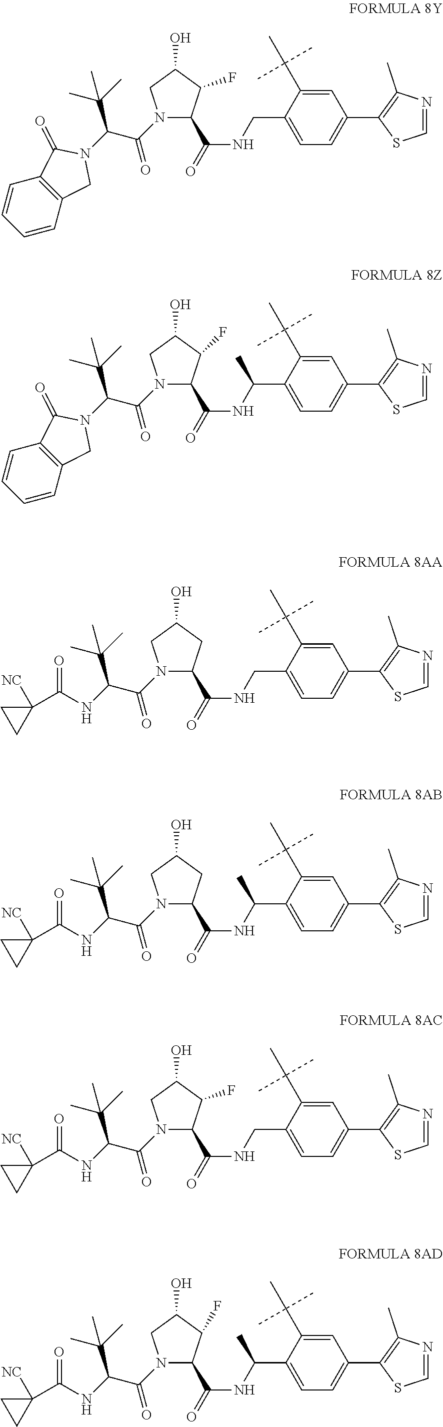

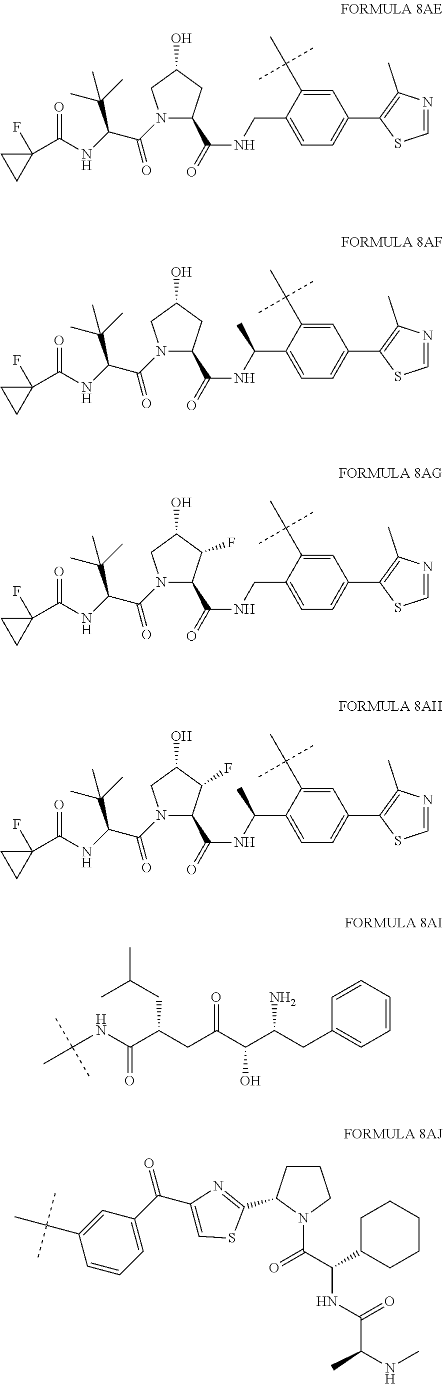

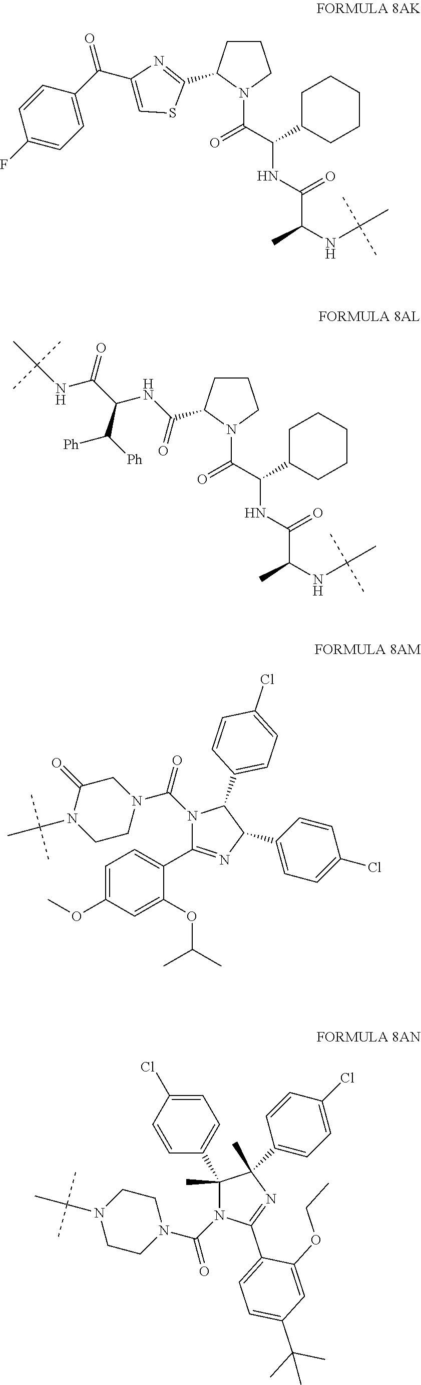

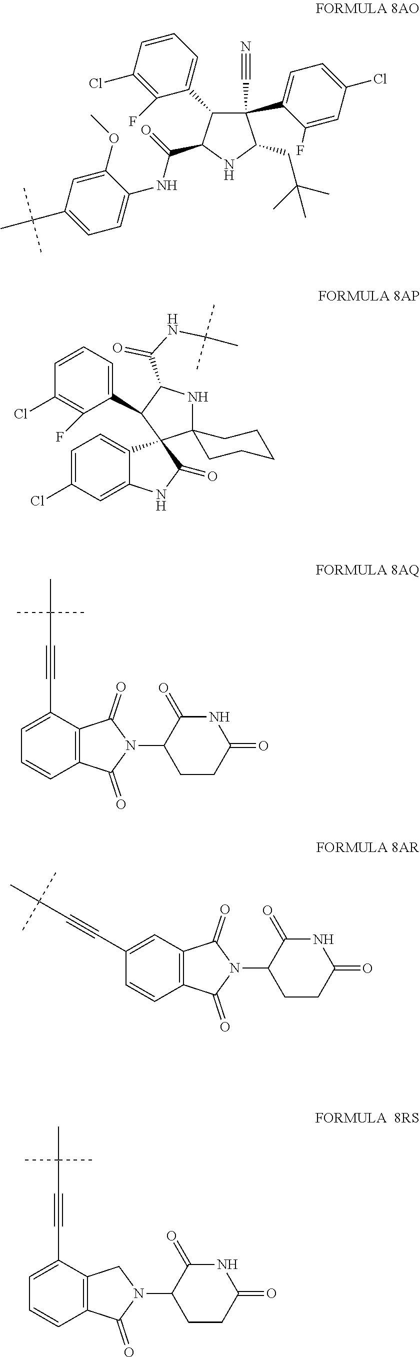

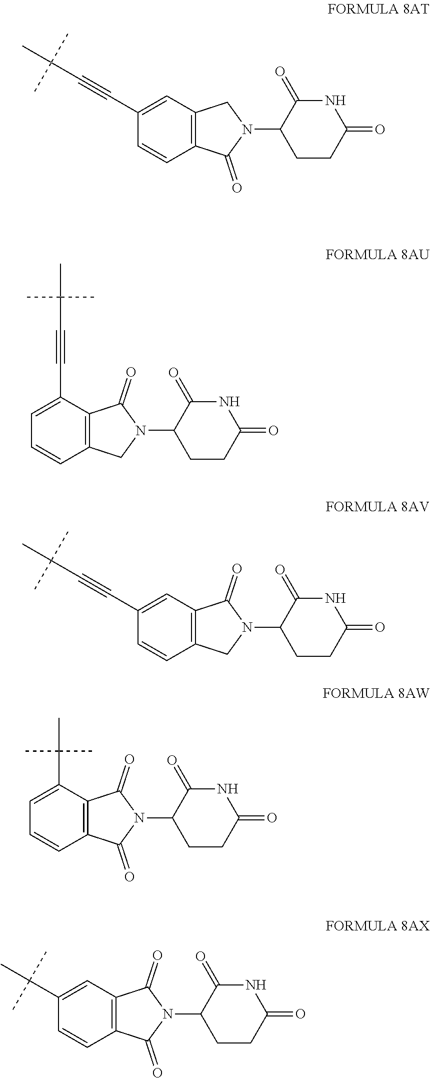

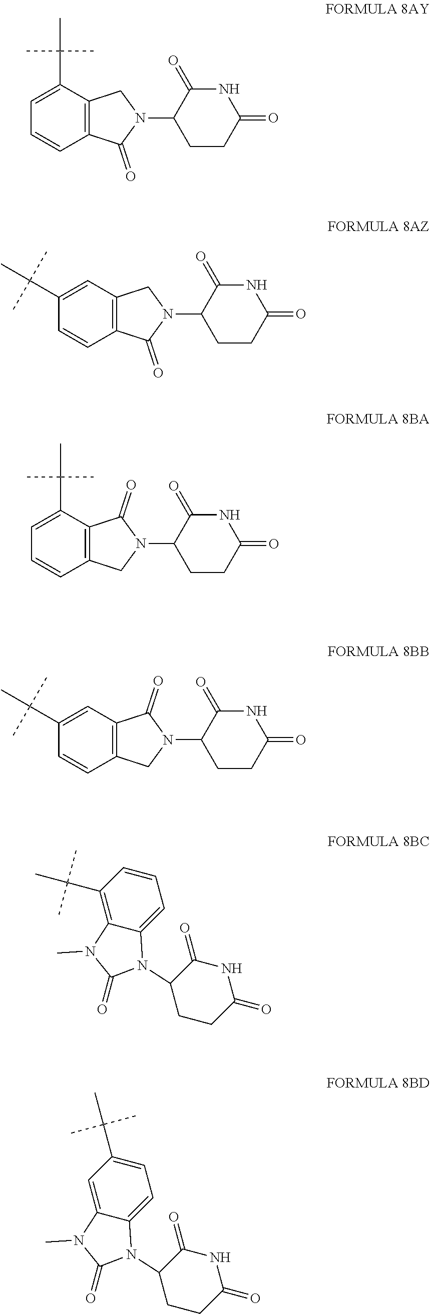

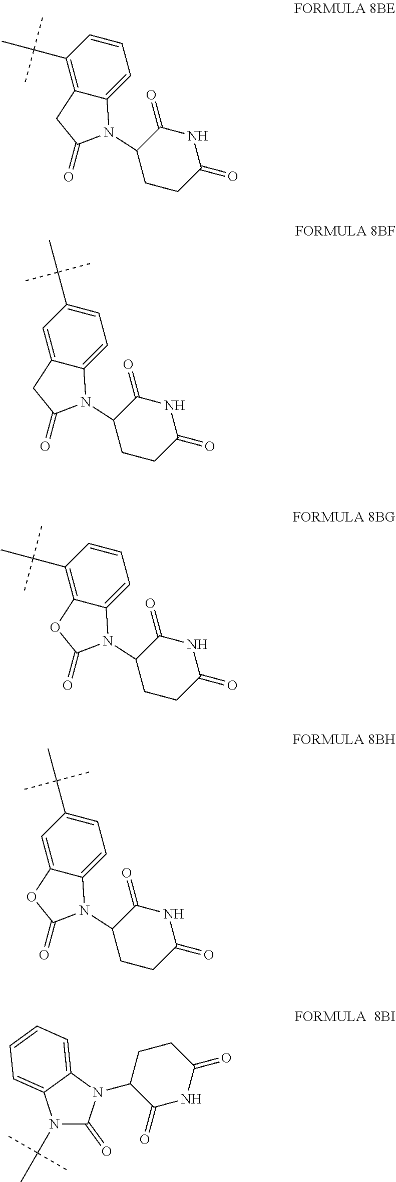

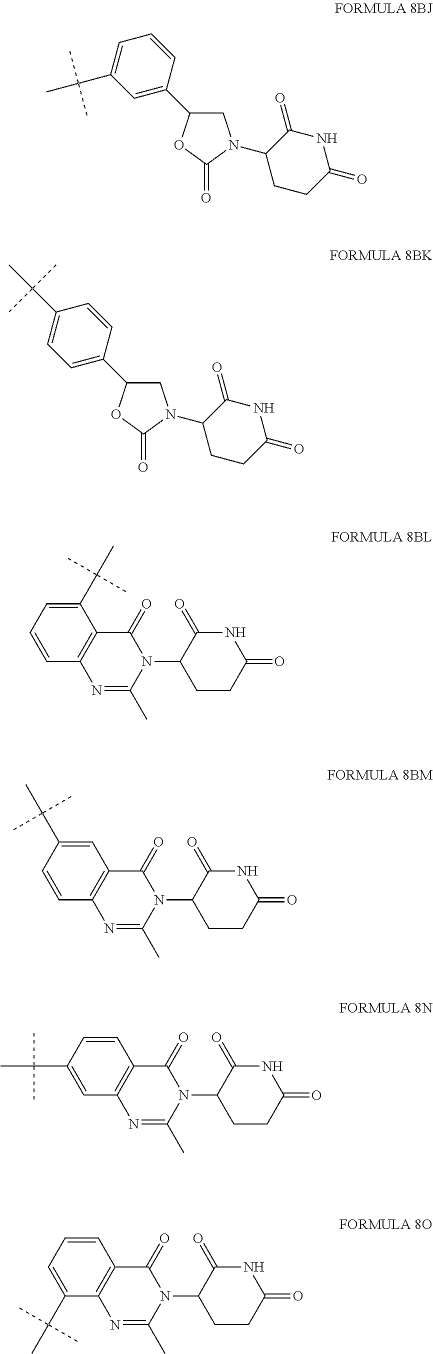

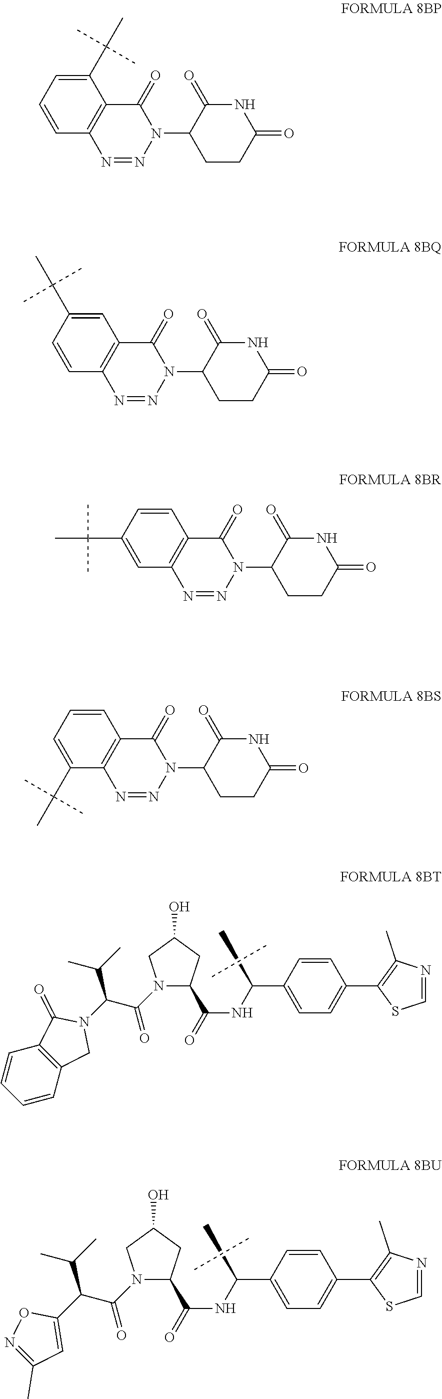

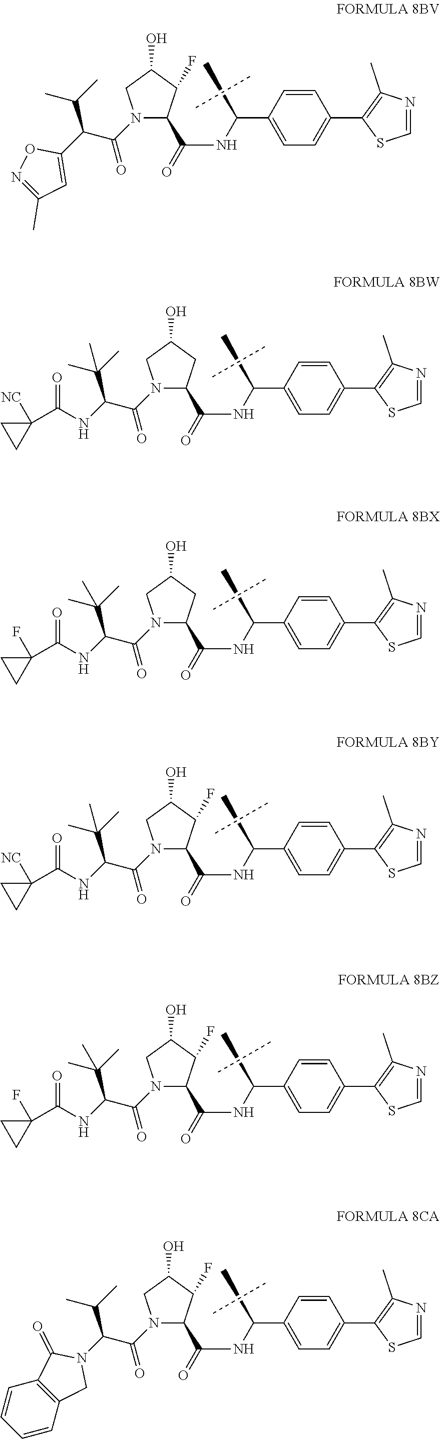

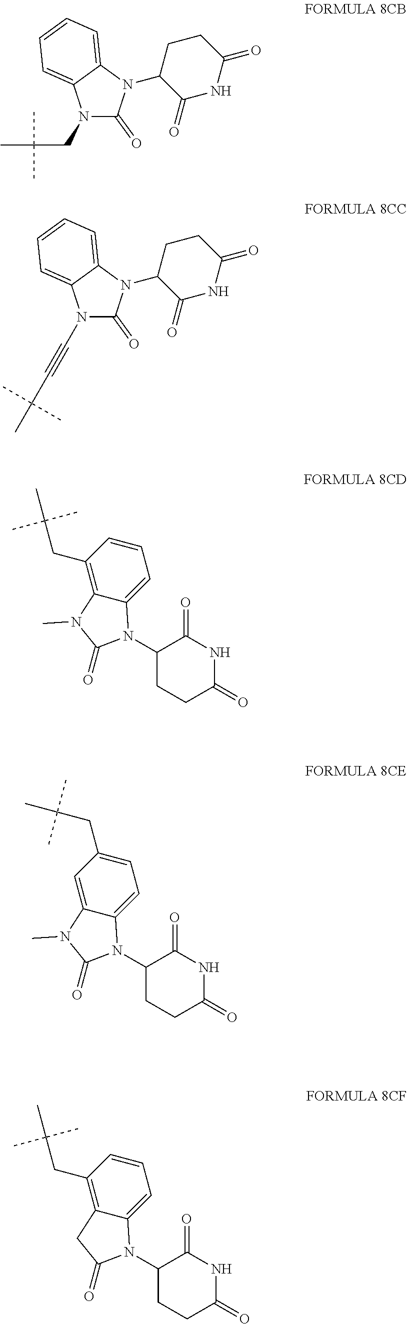

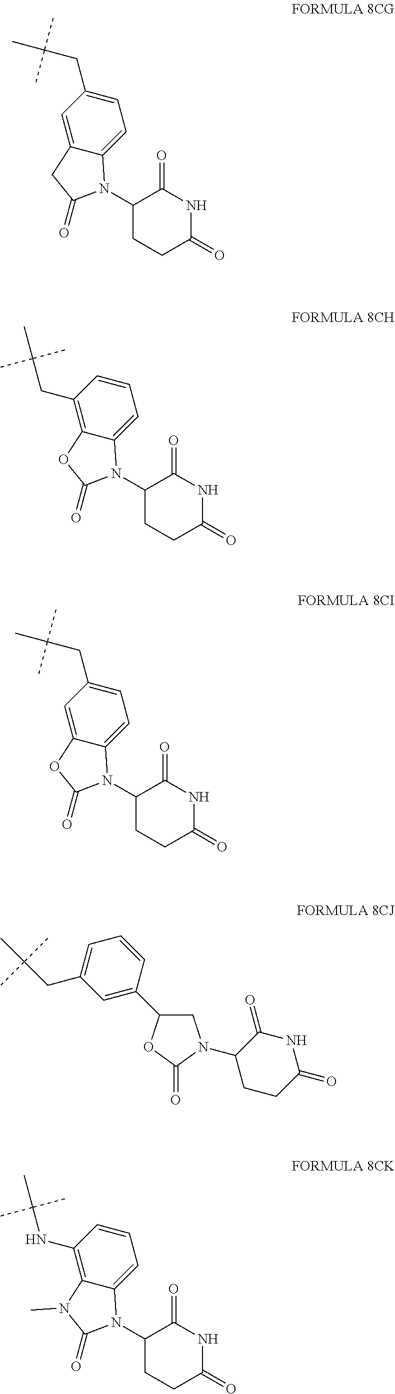

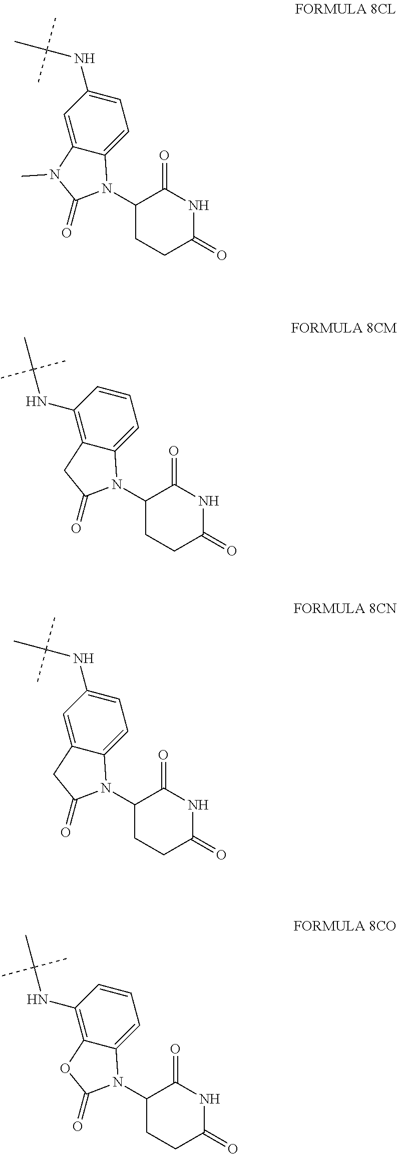

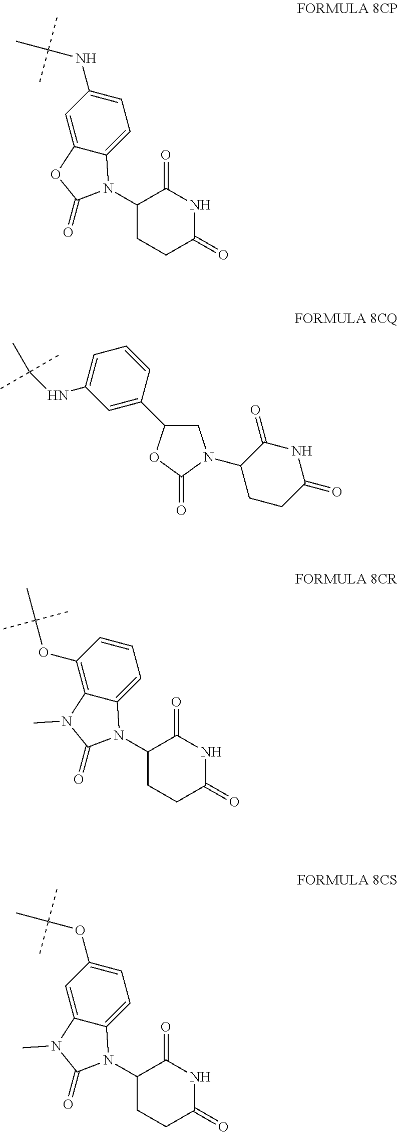

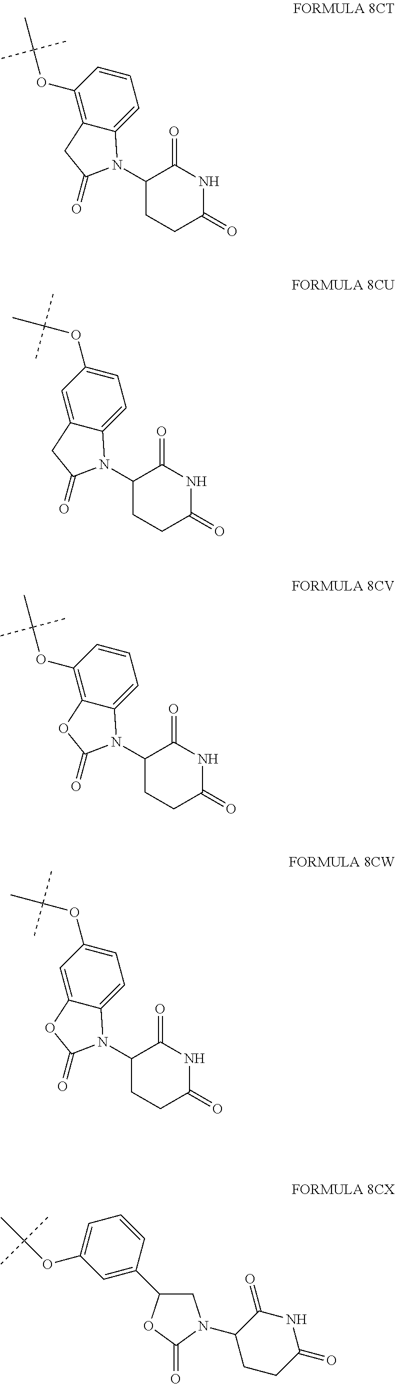

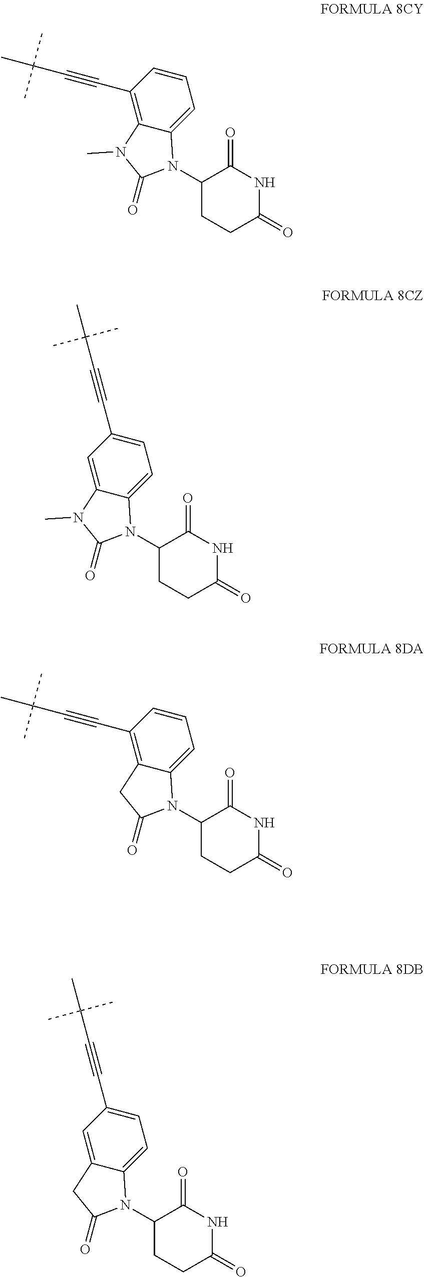

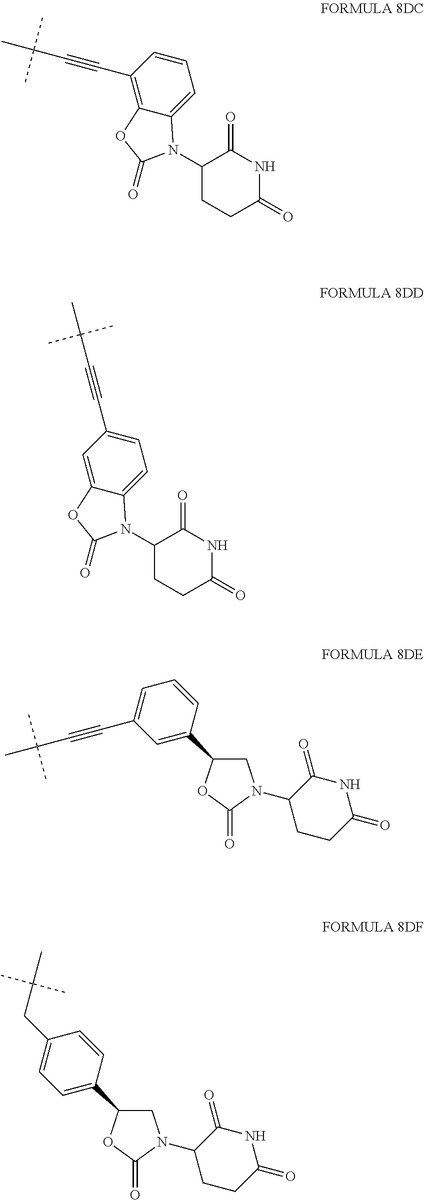

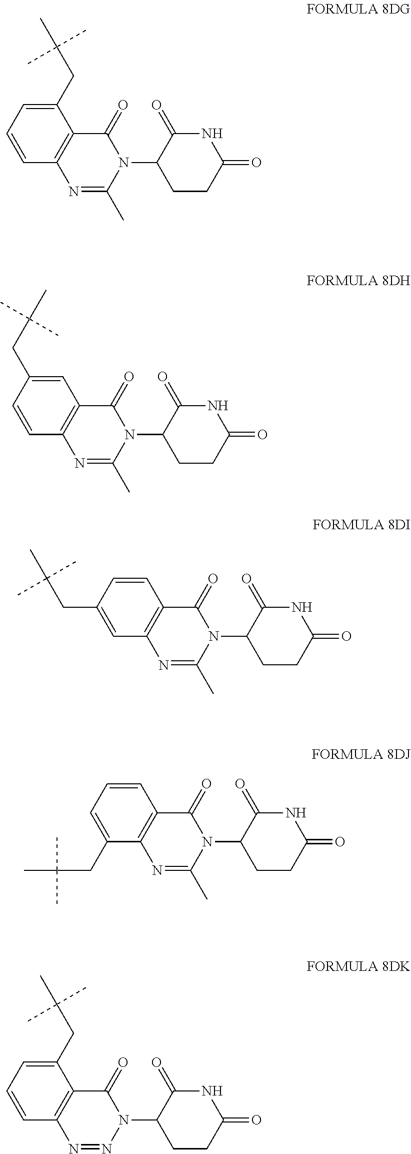

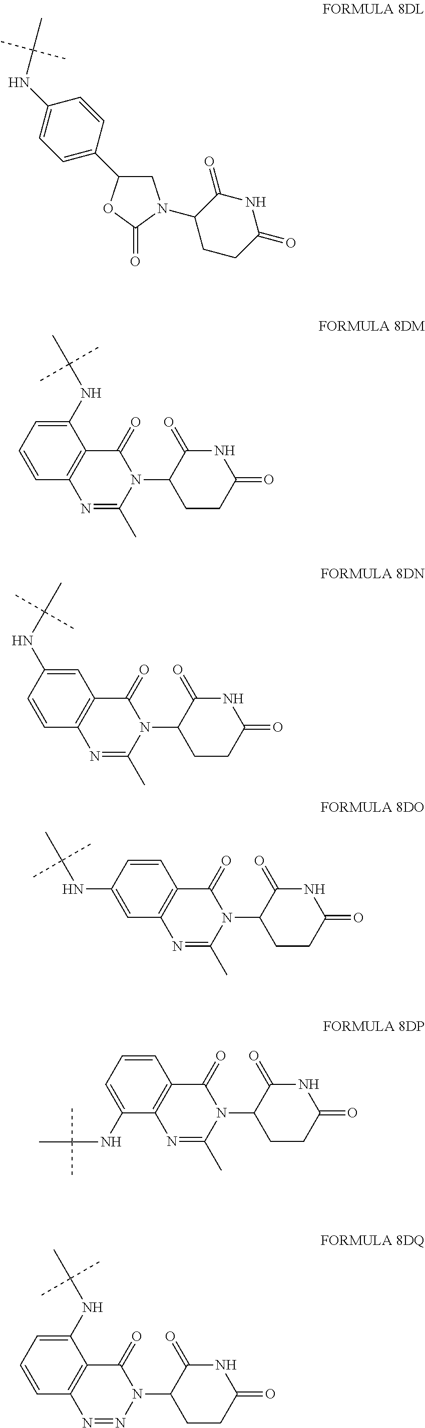

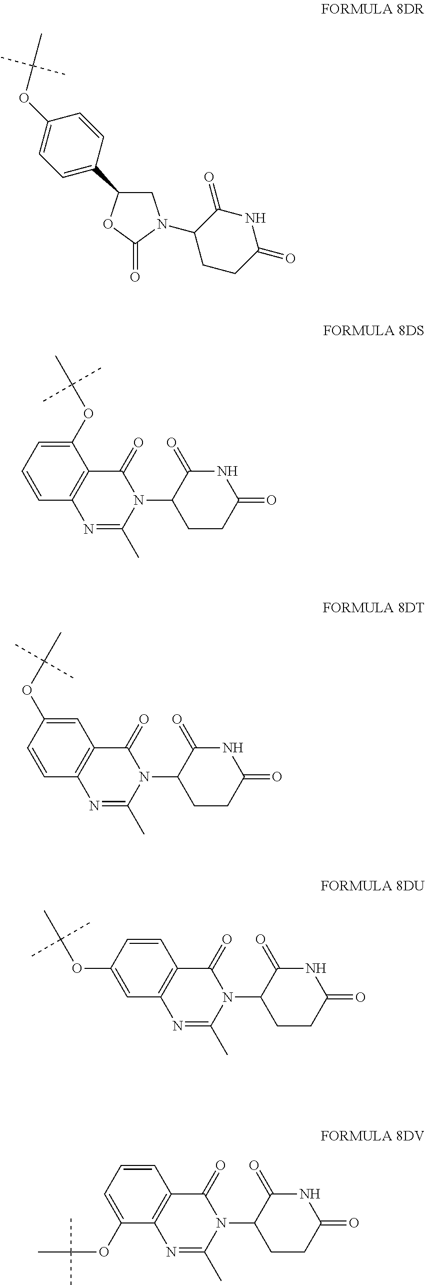

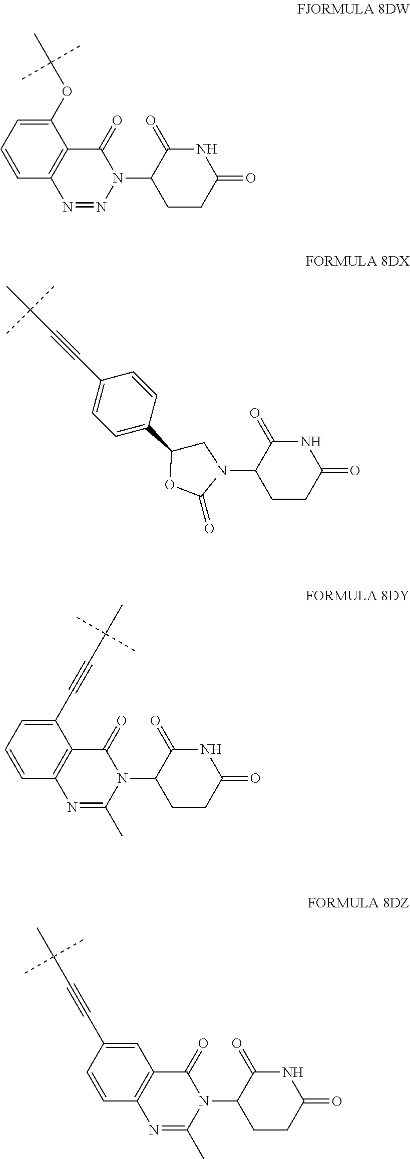

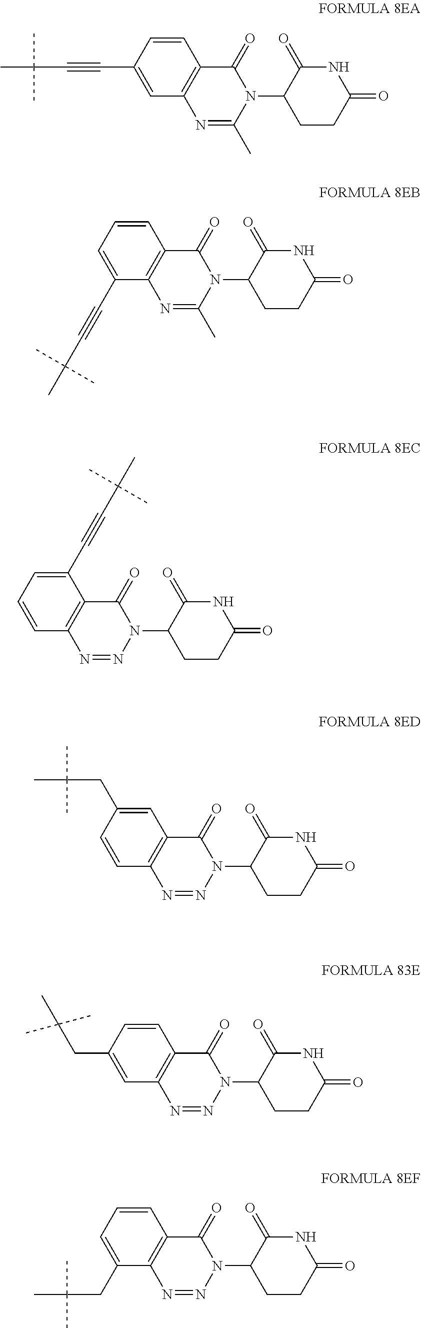

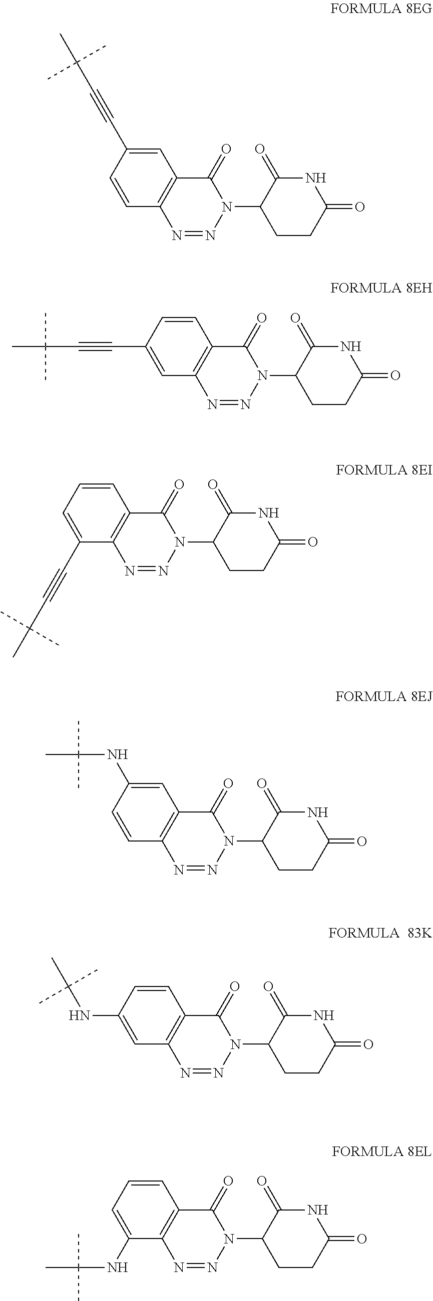

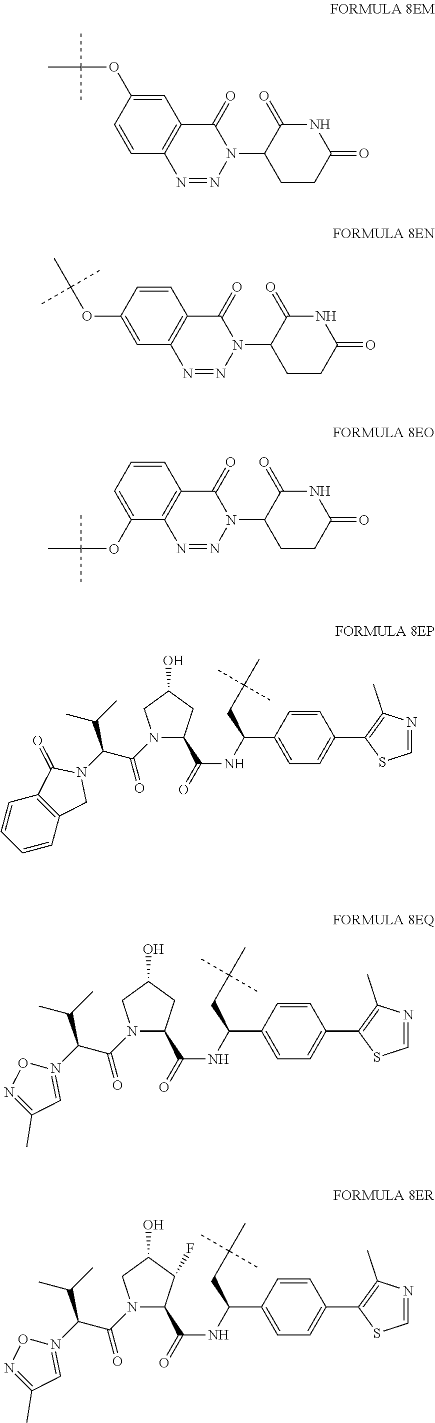

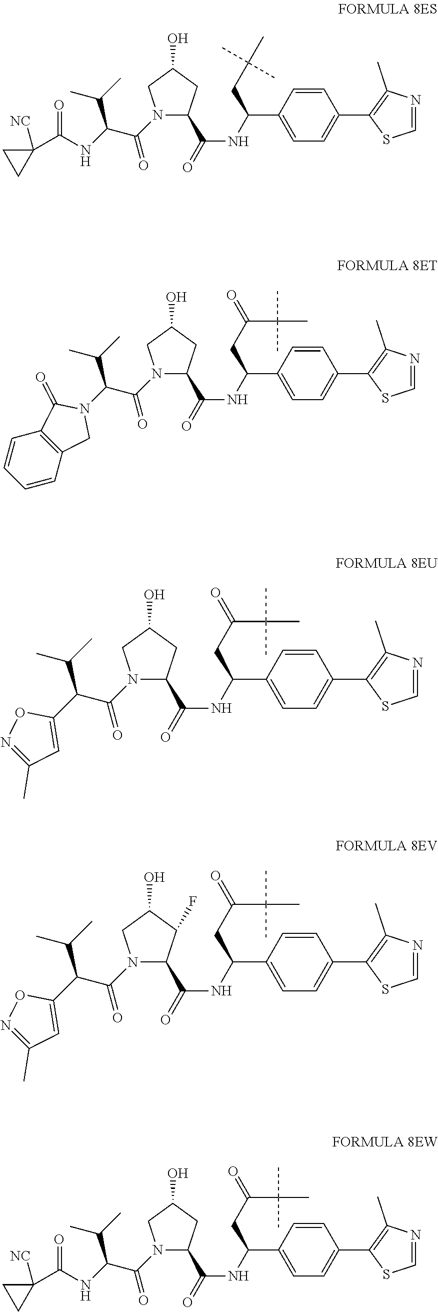

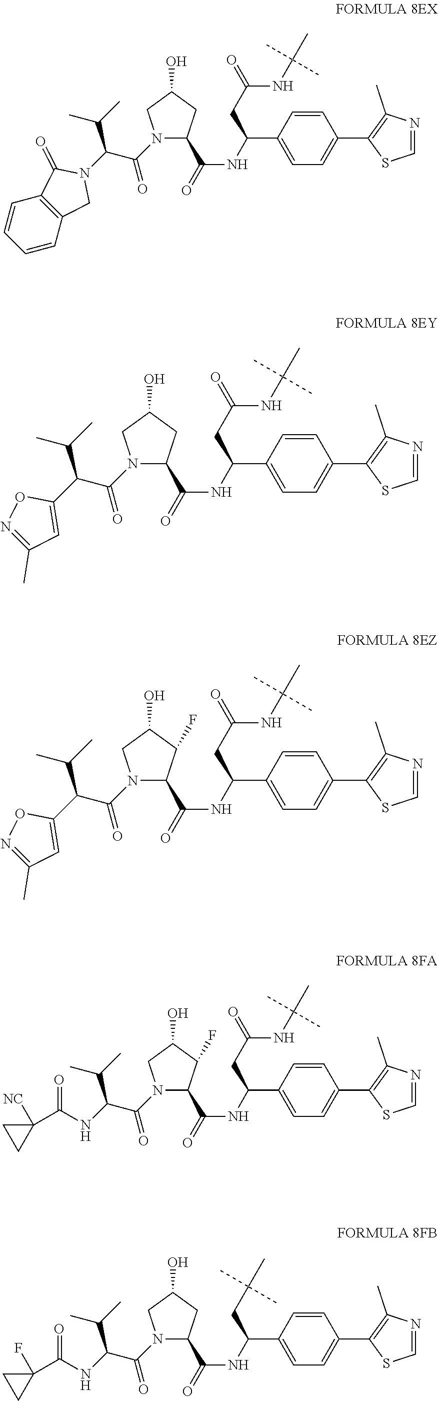

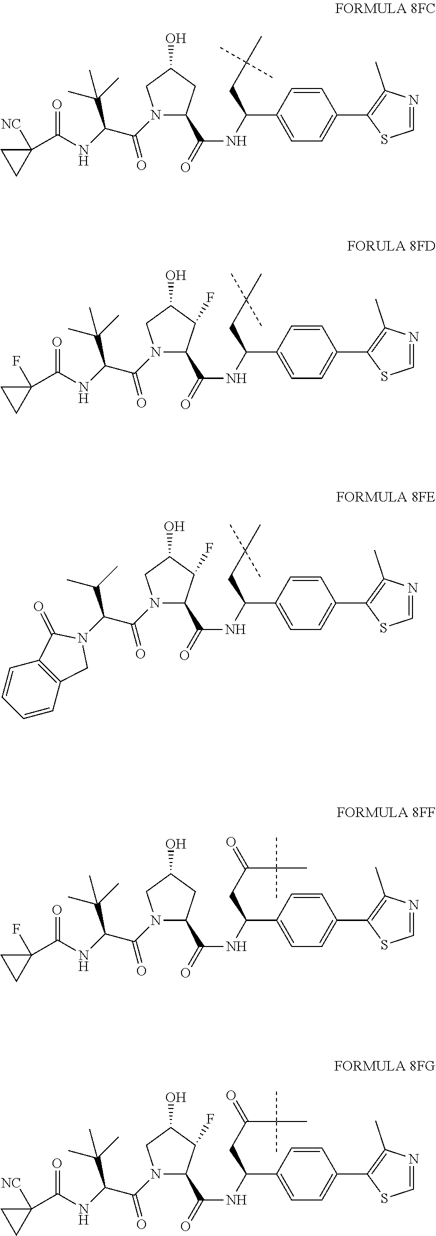

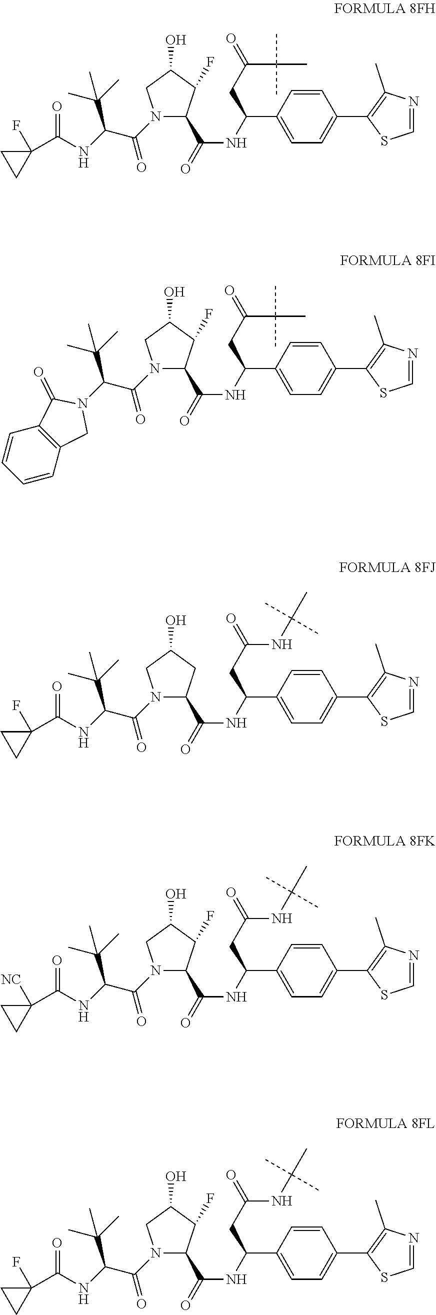

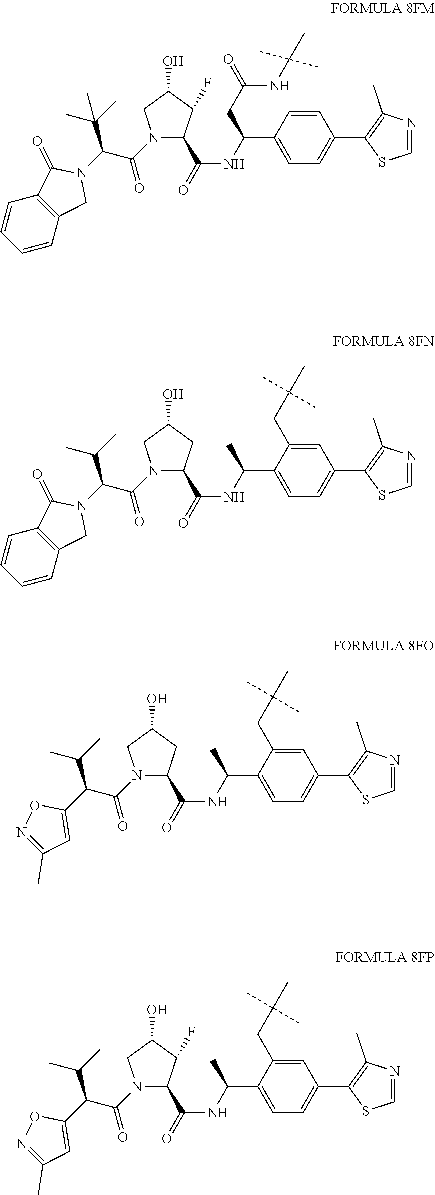

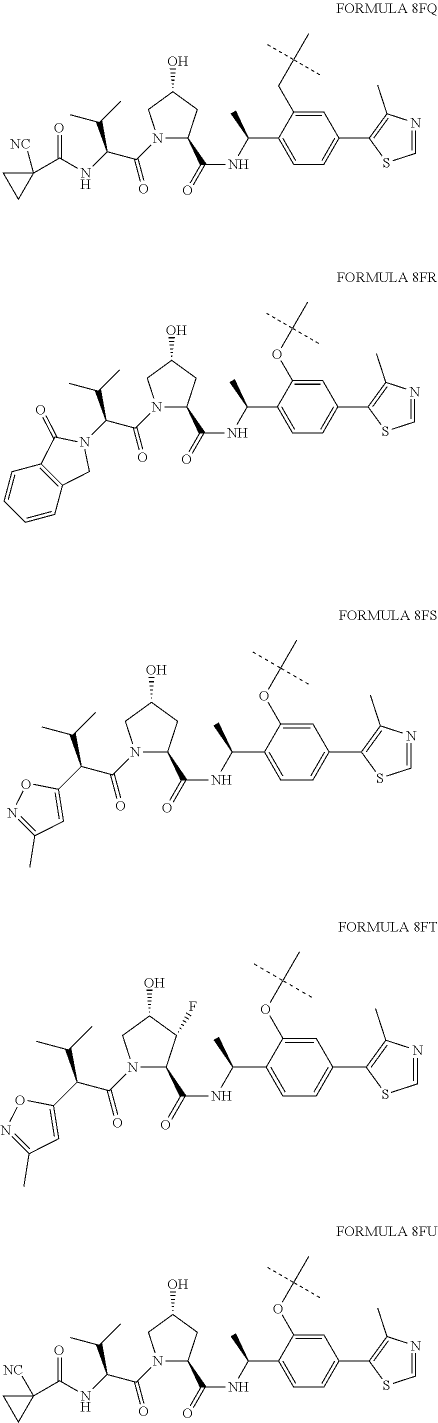

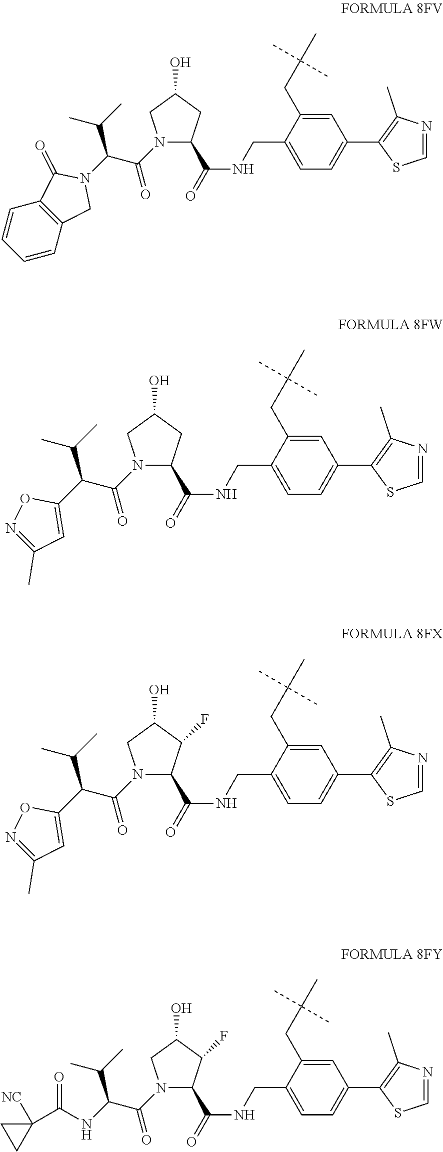

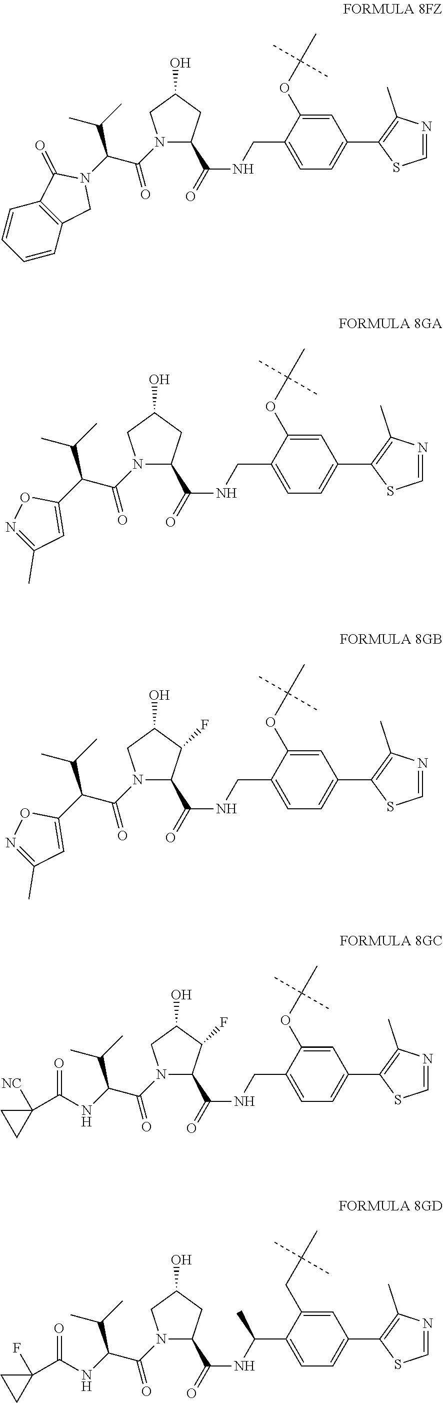

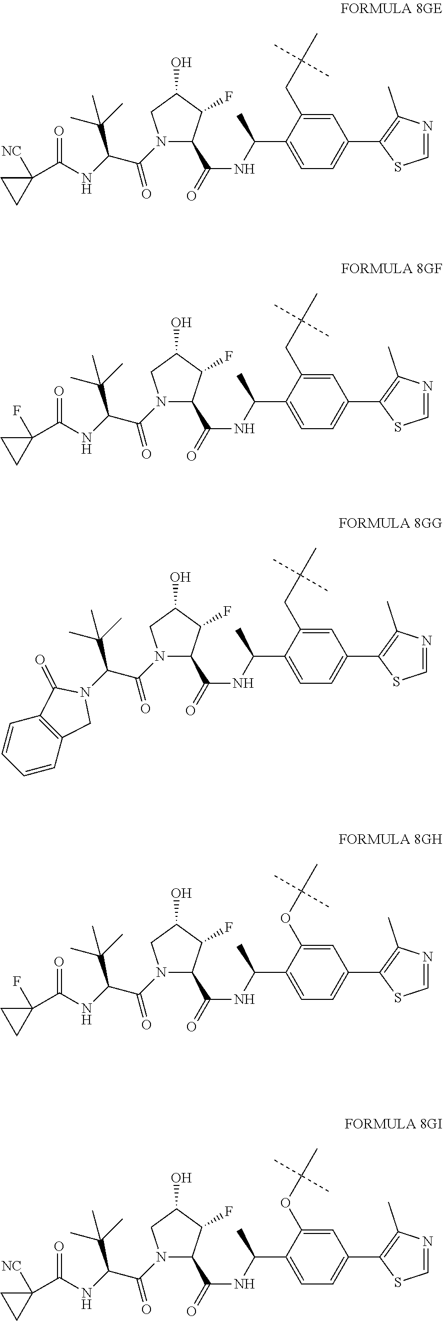

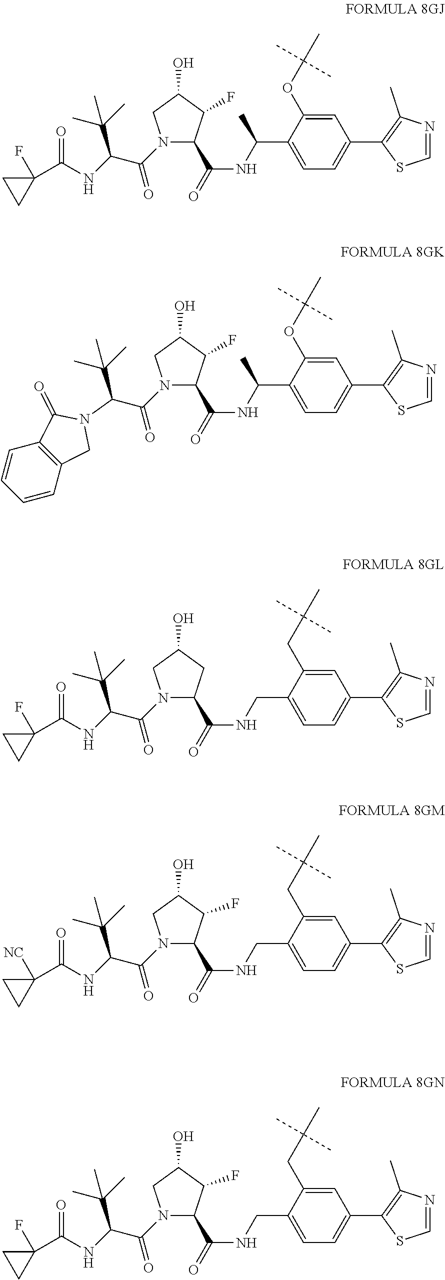

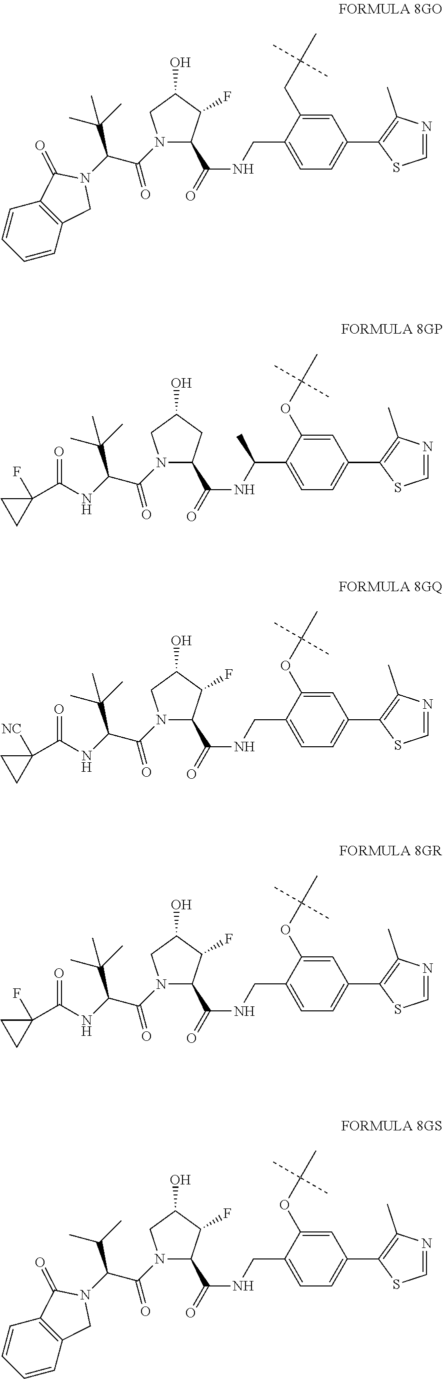

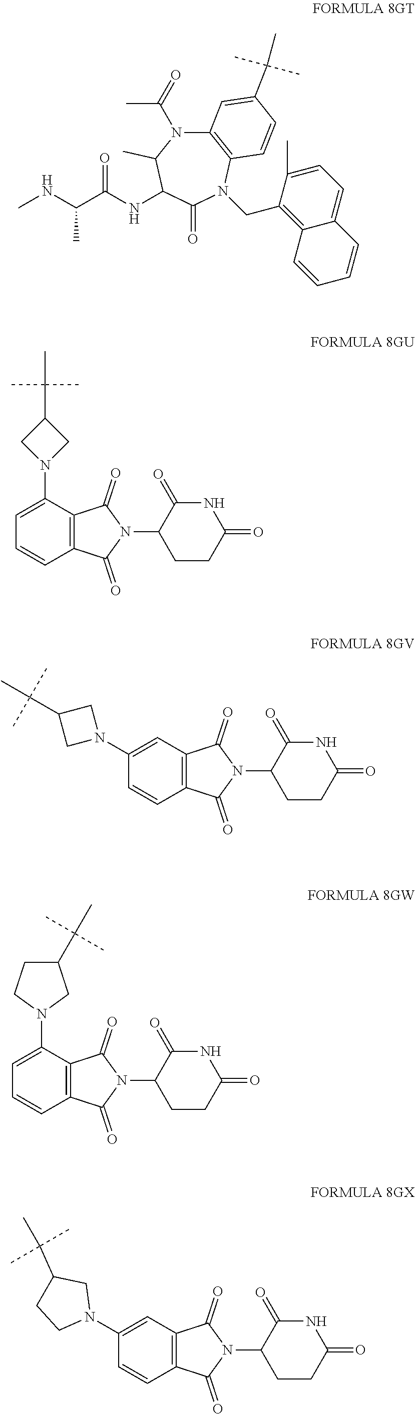

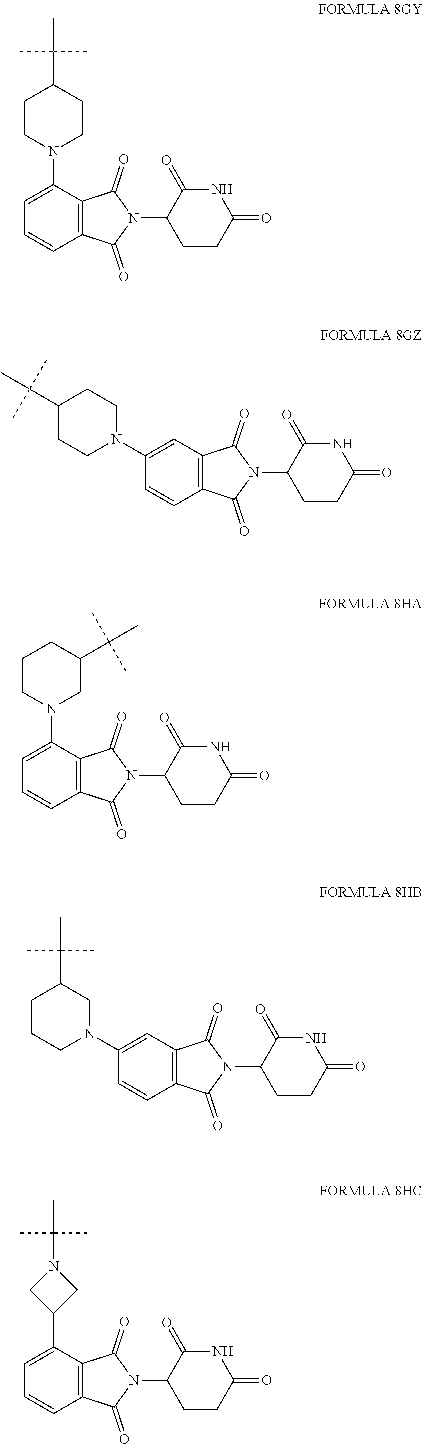

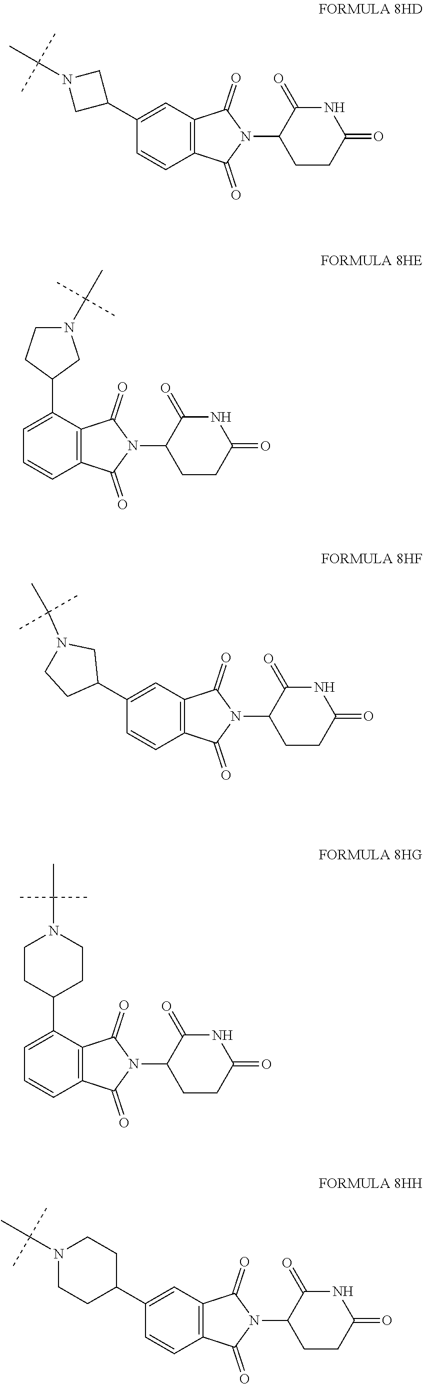

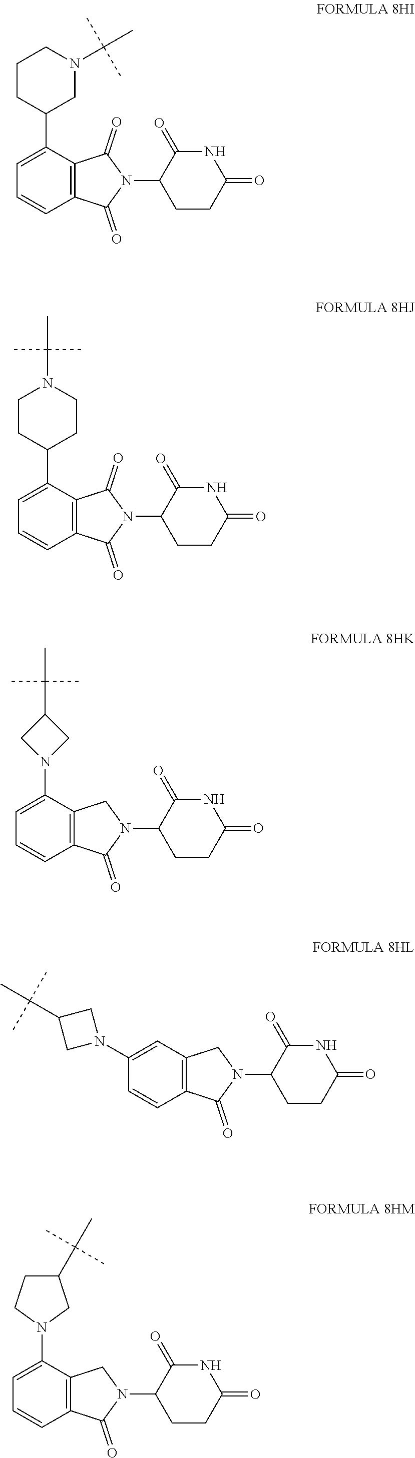

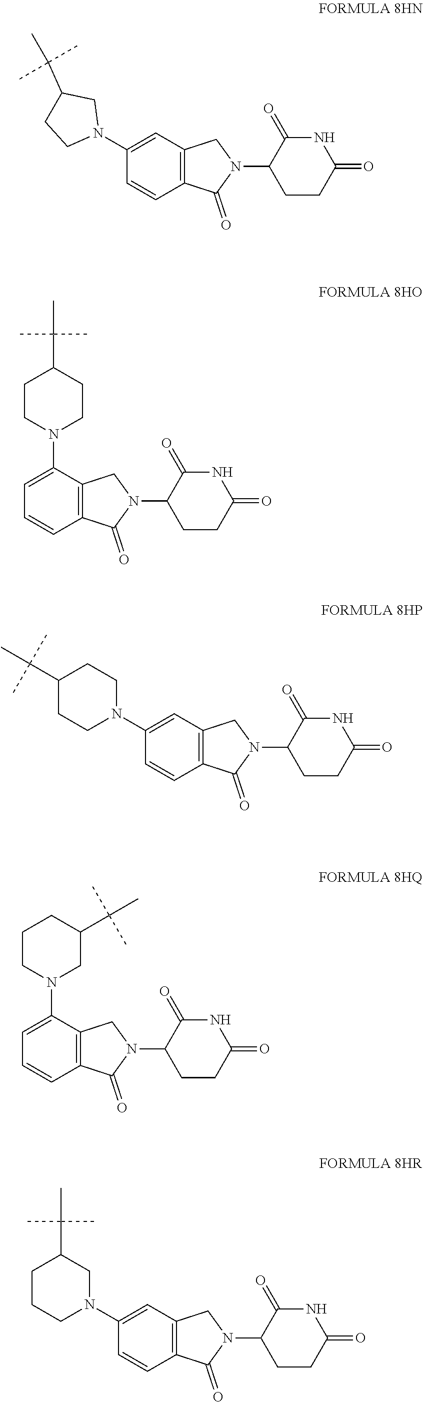

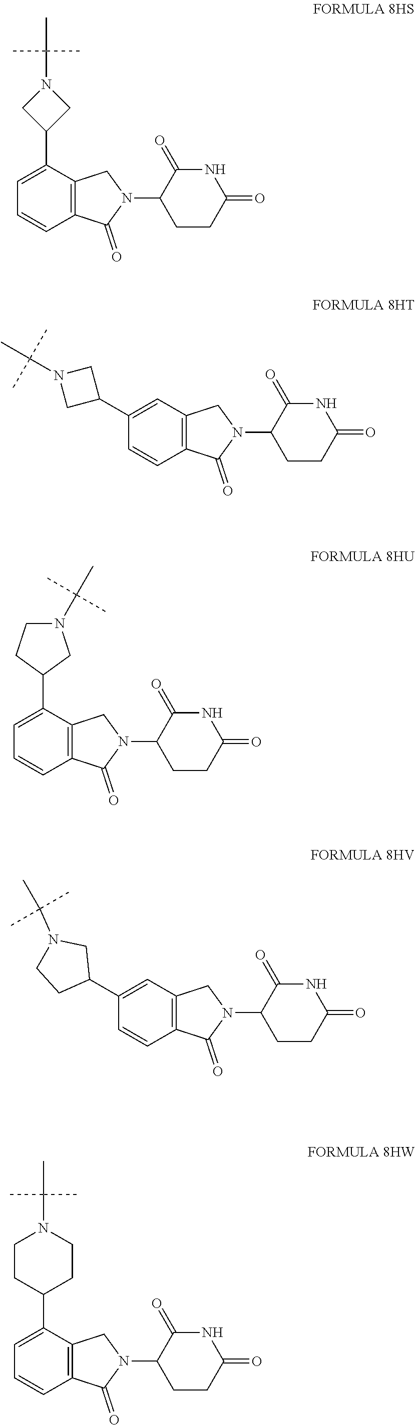

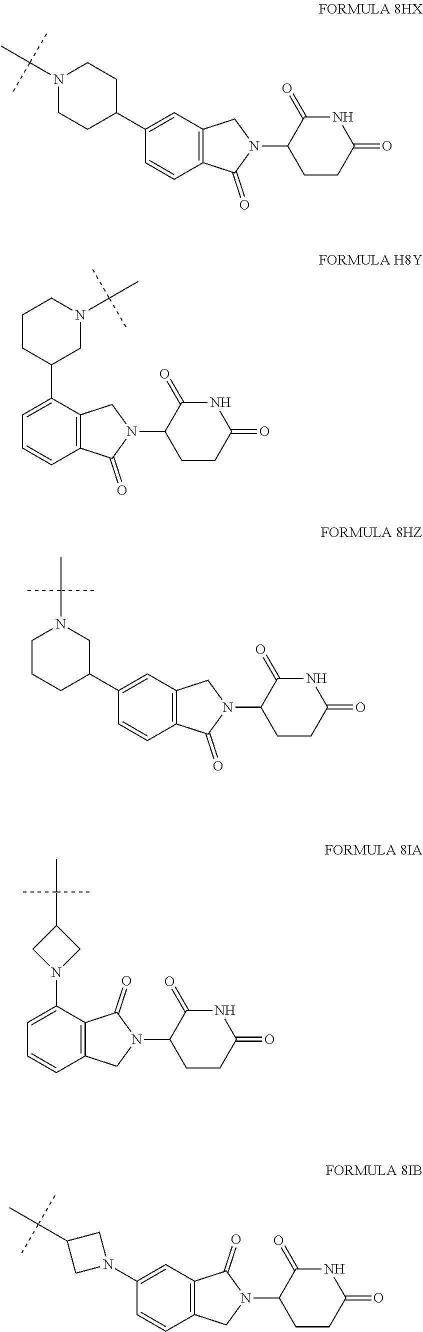

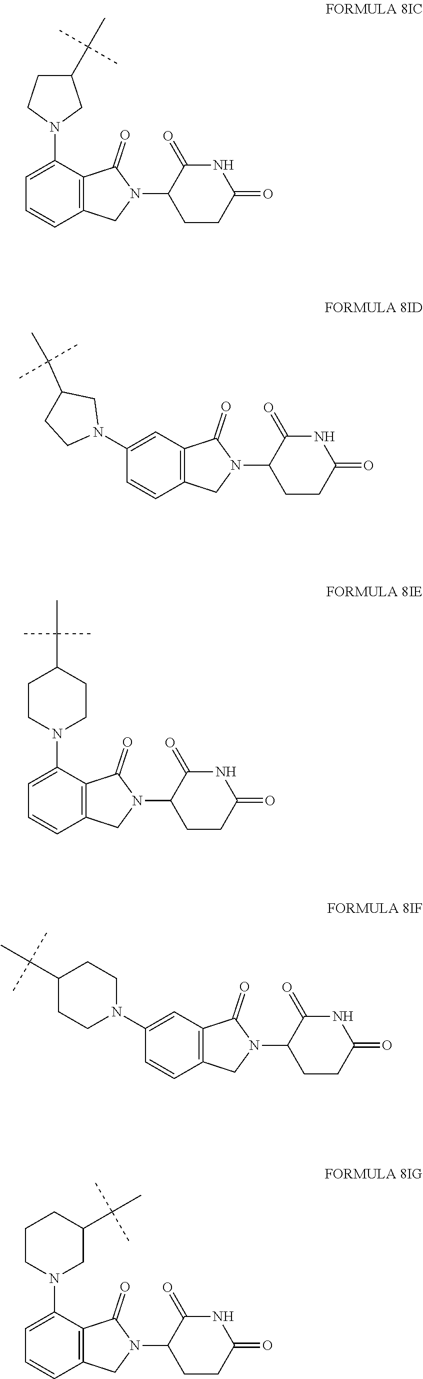

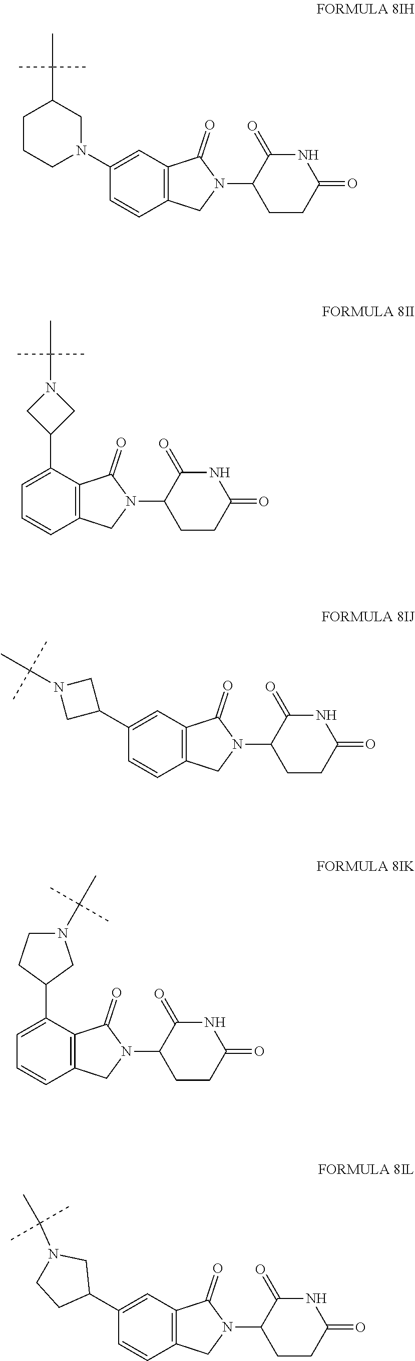

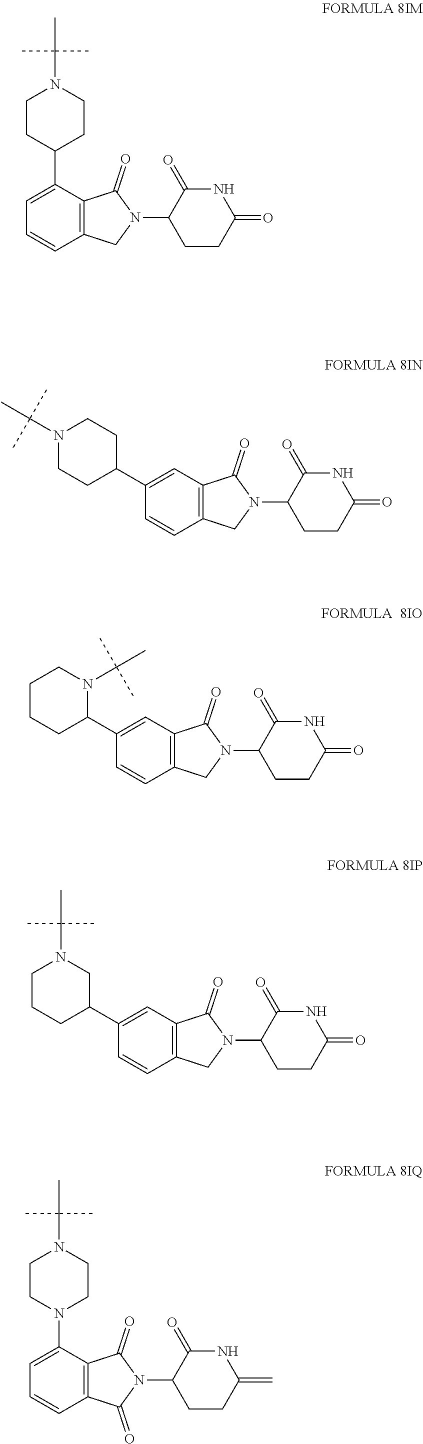

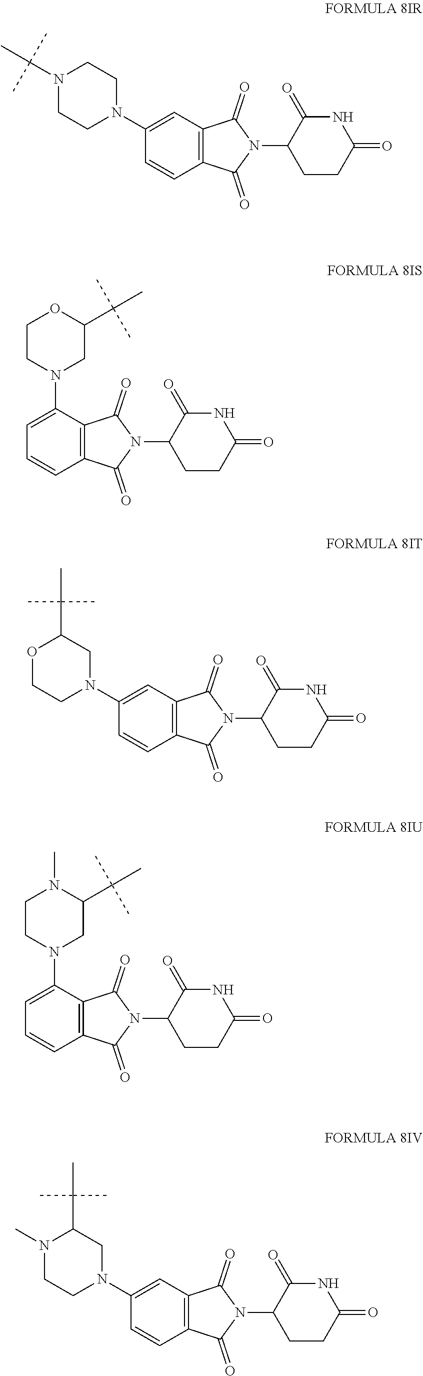

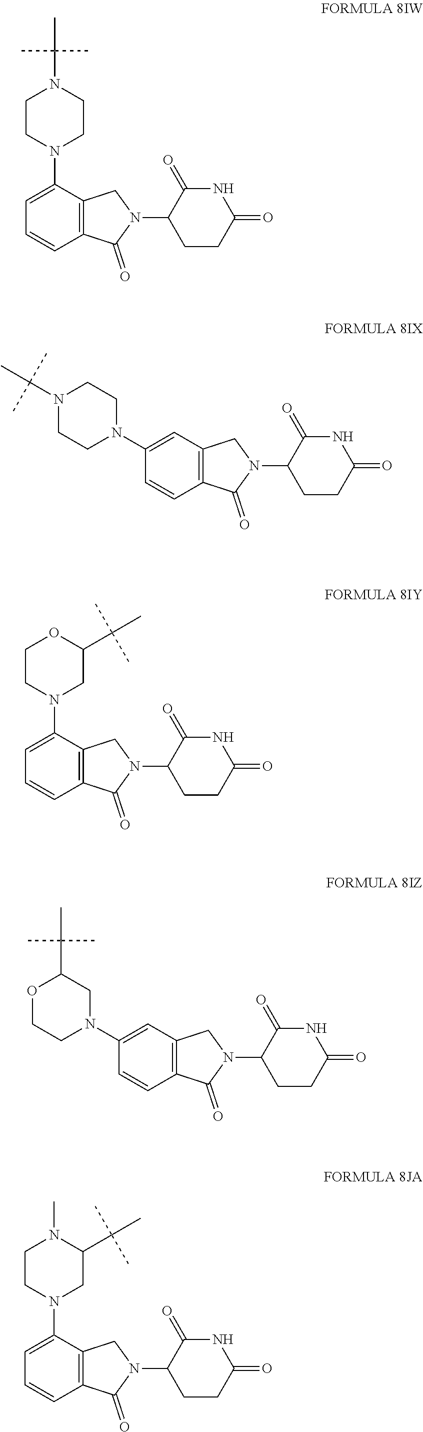

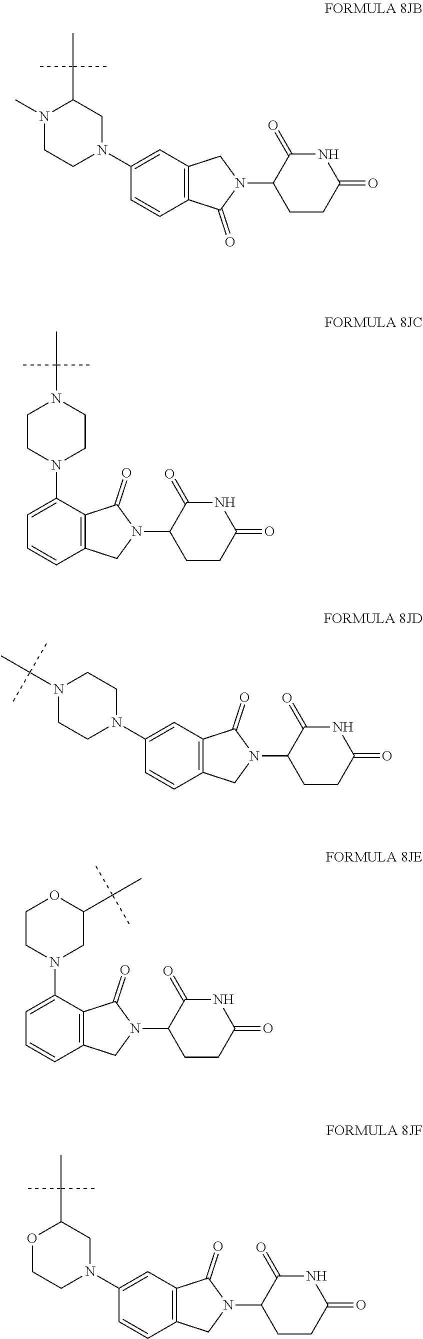

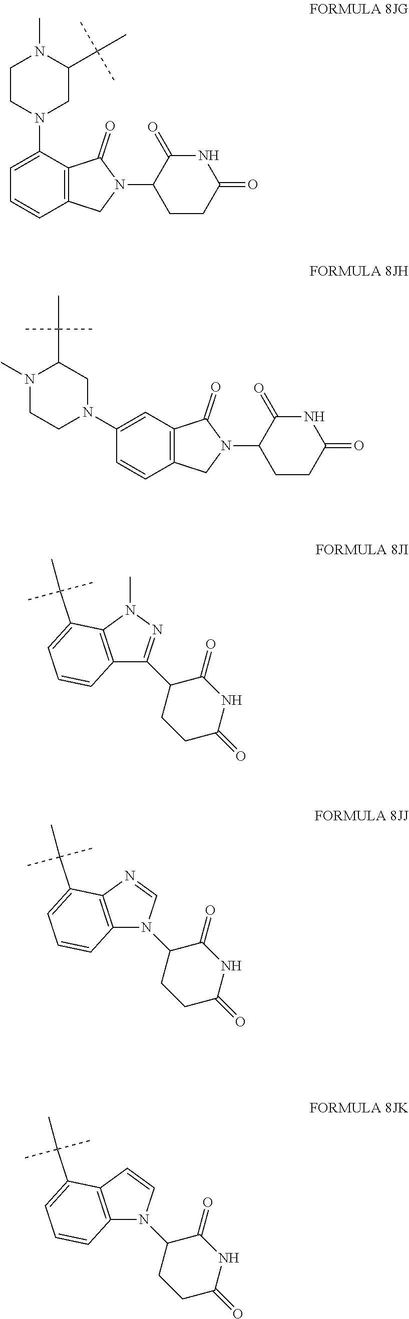

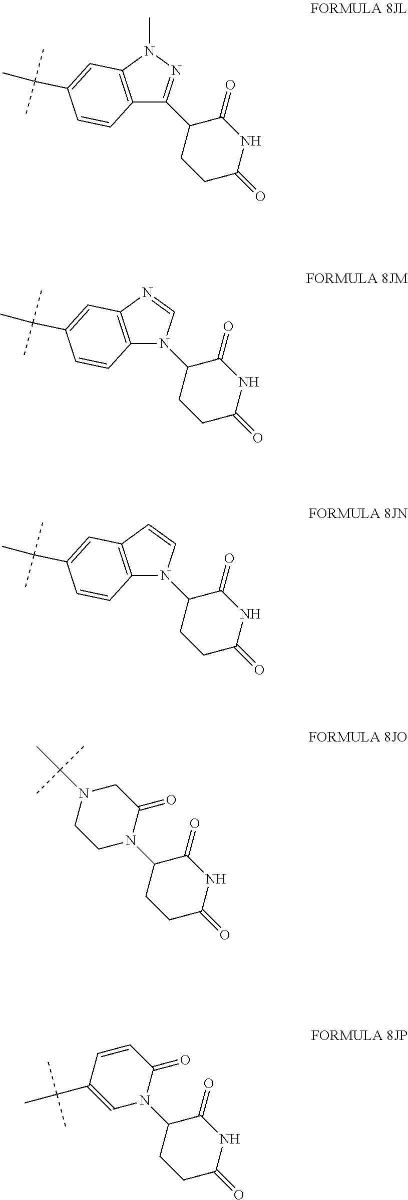

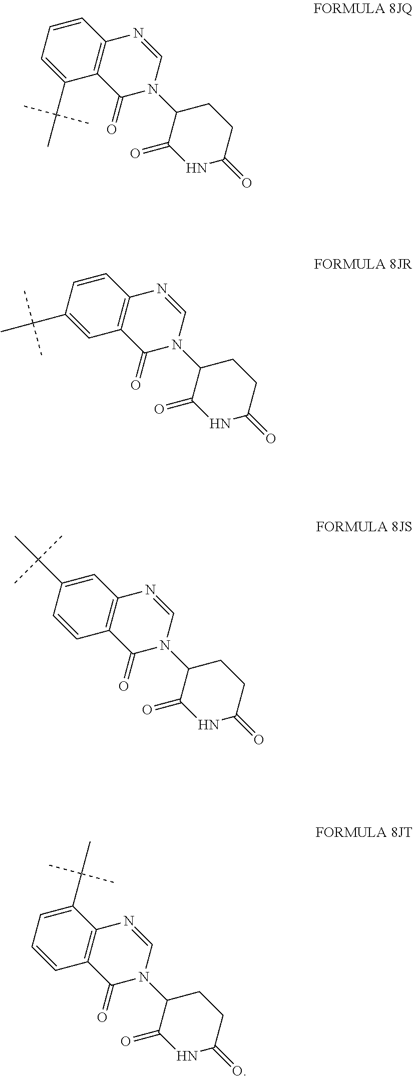

In another embodiment, the degradation tag is selected from the group consisting of: FORMULA 8A, 8B, 8C, 8D, 8E, 8F, 8G, 8H, 8I, 8J, 8K, 8L, 8M, 8O, 8P, 8Q, 8R, 8AQ, 8AR, 8AS, 8AT, 8AU, 8AV, 8AW, 8AX, 8AY, 8AZ, 8BA, 8BB, 8BC, 8BD, 8BE, 8BF, 8BG, 8BH, 8BI, 8BJ, 8BK, 8BL, 8BM, and 8BN, 8BO, 8BP, 8BQ, 8BR, 8BS, 8CB, 8CC, 8CD, 8CE, 8CF, 8CG, 8CH, 8CI, 8CJ, 8CK, 8CL, 8CM, 8CN, 8CO, 8CP, 8CQ, 8CR, 8CS, 8CT, 8CU, 8CV, 8CW, 8CX, 8CY, 8CZ, 8DA, 8DB, 8DC, 8DD, 8DE, 8DF, 8DG, 8DH, 8DI, 8DJ, 8DK, 8DL, 8DM, 8DN, 8DO, 8DP, 8DQ, 8DR, 8DS, 8DT, 8DU, 8DV, 8DW, 8DX, 8DY, 8DZ, 8EA, 8EB, 8EC, 8ED, 8EE, 8EF, 8EG, 8EH, 8EI, 8EJ, 8EK, 8EL, 8EM, 8EN, 8EO, 8EP, 8EO, 8GU, 8GV, 8GW, 8GX, 8GY, 8GZ, 8HA, 8HB, 8HC, 8HD, 8HE, 8HF, 8HG, 8HH, 8HI, 8HJ, 8HK, 8HL, 8HM, 8HN, 8HO, 8HP, 8HQ, 8HR, 8HS, 8HT, 8HU, 8HV, 8HW, 8HX, 8HY, 8HZ, 8IA, 8IB, 8IC, 8ID, 8IE, 8IF, 8IG, 8IH, 8II, 8IJ, 8IK, 8IL, 8IM, 8IN, 8IO, 8IP, 8IQ, 8IR, 8IS, 8IT, 8IU, 8IV, 8IW, 8IX, 8IY, 8IZ, 8JA, 8JB, 8JC, 8JD, 8JE, 8JF, 8JG, 8JH, 8JI, 8JJ, 8JK, 8JL, 8JM, 8JN, 8JO, 8JP, 8JQ, 8JR, 8JS, and 8JT.

Linker Moiety

As used herein, a “linker” or “linker moiety” is a bond, molecule, or group of molecules that binds two separate entities to one another. Linkers provide for optimal spacing of the two entities.

The term “linker” in some aspects refers to any agent or molecule that bridges the CBP/P300 ligand to the degradation tag. One of ordinary skill in the art recognizes that sites on the CBP/P300 ligand or the degradation tag, which are not necessary for the function of the PROTACs or SNIPERs of the present disclosure, are ideal sites for attaching a linker, provided that the linker, once attached to the conjugate of the present disclosures, does not interfere with the function of the CBP/P300 ligand, i.e., its ability to bind CBP/P300, or the function of the degradation tag, i.e., its ability to recruit a ubiquitin ligase.

The length of the linker of the bivalent compound can be adjusted to minimize the molecular weight of the bivalent compounds, avoid the clash of the CBP/P300 ligand or targeting moiety with the ubiquitin ligase and/or induce CBP/P300 misfolding by the hydrophobic tag. In certain embodiments, the linker comprises acyclic or cyclic saturated or unsaturated carbon, ethylene glycol, amide, amino, ether, urea, carbamate, aromatic, heteroaromatic, heterocyclic or carbonyl groups. In certain embodiments, the length of the linker is 0, 1, 2, 3, 4, 5, 6, 7, 8, 9, 10, 11, 12, 13, 14, 15, 16, 17, 18, 19, 20 or more atoms.

In some embodiments, the linker moiety is of FORMULA 9:

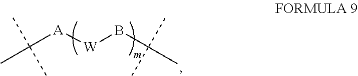

wherein

A, W and B, at each occurrence, are independently selected from null, or bivalent moiety selected from R′-R″, R′COR″, R′CO₂R″, R′C(O)N(R¹)R″, R′C(S)N(R¹)R″, R′OR″, R′SR″, R′SOR″, R′SO₂R″, R′SO₂N(R¹)R″, R′N(R¹)R″, R′N(R¹)COR″, R′N(R¹)CON(R²)R″, R′N(R¹)C(S)R″, optionally substituted C₁-C₈ alkylene, optionally substituted C₂-C₈ alkenylene, optionally substituted C₂-C₈ alkynylene, optionally substituted C₁-C₈alkoxyC₁-C₈alkylene, optionally substituted C₁-C₈ haloalkylene, optionally substituted C₁-C₈ hydroxyalkylene, optionally substituted C₃-C₁₃ fused cycloalkyl, optionally substituted C₃-C₁₃ fused heterocyclyl, optionally substituted C₃-C₁₃ bridged cycloalkyl, optionally substituted C₃-C₁₃ bridged heterocyclyl, optionally substituted C₃-C₁₃ spiro cycloalkyl, optionally substituted C₃-C₁₃ spiro heterocyclyl, optionally substituted 3-10 membered carbocyclyl, optionally substituted 4-10 membered heterocyclyl, optionally substituted aryl, and optionally substituted heteroaryl, wherein

R′ and R″ are independently selected from null, optionally substituted (C₁-C₈ alkylene)-R^{r} (preferably, CH₂-R^{r}), optionally substituted R^{r}—(C₁-C₈ alkylene), optionally substituted (C₁-C₈ alkylene)-R^{r}—(C₁-C₈ alkylene), or a moiety comprising of optionally substituted C₁-C₈ alkyl, optionally substituted C₂-C₈ alkenyl, optionally substituted C₂-C₈ alkynyl, optionally substituted C₁-C₈ hydroxyalkyl, optionally substituted C₁-C₈alkoxyC₁-C₈alkyl, optionally substituted C₁-C₈alkylaminoC₁-C₈alkyl, optionally substituted C₁-C₈ haloalkyl, optionally substituted C₁-C₈ alkylene, optionally substituted C₂-C₈ alkenylene, optionally substituted C₂-C₈ alkynylene, optionally substituted C₁-C₈ hydroxyalkylene, optionally substituted C₁-C₈alkoxyC₁-C₈alkylene, optionally substituted C₁-C₈alkylaminoC₁-C₈alkylene, optionally substituted C₁-C₈ haloalkylene, optionally substituted 3-10 membered carbocyclyl, optionally substituted 4-10 membered heterocyclyl, optionally substituted C₃-C₁₃ fused cycloalkyl, optionally substituted C₃-C₁₃ fused heterocyclyl, optionally substituted C₃-C₁₃ bridged cycloalkyl, optionally substituted C₃-C₁₃ bridged heterocyclyl, optionally substituted C₃-C₁₃ spiro cycloalkyl, optionally substituted C₃-C₁₃ spiro heterocyclyl, optionally substituted aryl, and optionally substituted heteroaryl;

R^{r} is selected from optionally substituted 3-10 membered carbocyclyl, optionally substituted 4-10 membered heterocyclyl, optionally substituted C₃-C₁₃ fused cycloalkyl, optionally substituted C₃-C₁₃ fused heterocyclyl, optionally substituted C₃-C₁₃ bridged cycloalkyl, optionally substituted C₃-C₁₃ bridged heterocyclyl, optionally substituted C₃-C₁₃ spiro cycloalkyl, optionally substituted C₃-C₁₃ spiro heterocyclyl, optionally substituted aryl, and optionally substituted heteroaryl;

R¹ and R² are independently selected from hydrogen, optionally substituted C₁-C₈ alkyl, optionally substituted C₂-C₈ alkenyl, optionally substituted C₂-C₈ alkynyl, optionally substituted C₁-C₈ alkoxyalkyl, optionally substituted C₁-C₈ haloalkyl, optionally substituted C₁-C₈ hydroxyalkyl, optionally substituted C₁-C₈alkylaminoC₁-C₈alkyl, optionally substituted 3-10 membered carbocyclyl, optionally substituted 4-10 membered heterocyclyl, optionally substituted aryl, and optionally substituted heteroaryl;

R′ and R″, R¹ and R², R′ and R¹, R′ and R², R″ and R¹, R″ and R² together with the atom to which they are connected form a 3-20 membered cycloalkyl or 4-20 membered heterocyclyl ring; and

m is 0 to 15.

In another embodiment, W and m are defined as above; and A and B, at each occurrence, are independently selected from null, CO, NH, NH—CO, CO—NH, —(CH₂)₀₋₈—, —(CH₂)₀₋₃—CO—(CH₂)₀₋₈—, (CH₂)₀₋₈—NH—CO, (CH₂)₀₋₈—CO—NH, NH—CO—(CH₂)₀₋₈, CO—NH—(CH₂)₀₋₈, (CH₂)₁₋₃—NH—(CH₂)₁₋₃—CO—NH, (CH₂)₁₋₃—NH—(CH₂)₁₋₃—NH—CO, —CO—NH, CO—NH—(CH₂)₁₋₃—NH—(CH₂)₁₋₃, (CH₂)₁₋₃—NH—(CH₂)₁₋₃, —(CH₂)₀₋₃—R^{r}—(CH₂)₀₋₃, —(CH₂)₀₋₃—(CO)—(CH₂)₀₋₃—R^{r}—(CH₂)₀₋₃—, —(CH₂)₀₋₃—(CO—NH)—(CH₂)₀₋₃—R^{r}—(CH₂)₀₋₃—, —(CH₂)₀₋₃—(NH—CO)—(CH₂)₀₋₃—R^{r}—(CH₂)₀₋₃—, and —(CH₂)₀₋₃—(NH)—(CH₂)₀₋₃—R^{r}—(CH₂)₀₋₃—.

In another embodiment, W and m are defined as above; and A and B, at each occurrence, are independently selected from null, CO, NH, NH—CO, CO—NH, —(CH₂)₀₋₈—, —(CH₂)₀₋₃—CO—(CH₂)₀₋₈—, (CH₂)₁₋₂—NH—CO, (CH₂)₁₋₂-CO—NH, NH—CO—(CH₂)₁₋₂, CO—NH—(CH₂)₁₋₂, (CH₂)₁₋₂—NH—(CH₂)₁₋₂—CO—NH, (CH₂)₁₋₂—NH—(CH₂)₁₋₂—NH—CO, —CO—NH, CO—NH—(CH₂)₁₋₂—NH—(CH₂)₁₋₂, (CH₂)₁₋₂—NH—(CH₂)₁₋₂, —(CH₂)₀₋₂—R^{r}—(CH₂)₀₋₂, —(CH₂)₀₋₂—(CO)—(CH₂)₀₋₃—Rᵣ—(CH₂)₀₋₂—, —(CH₂)₀₋₂—(CO—NH)—(CH₂)₀₋₃—Rᵣ—(CH₂)₀₋₂—, —(CH₂)₀₋₂—(NH—CO)—(CH₂)₀₋₃—Rᵣ—CH₂)₀₋₂—, —(CH₂)₀₋₂—(NH)—(CH₂)₀₋₃—R^{r}—(CH₂)₀₋₂—.

In another embodiment, R^{r} is selected from FORMULA C1a, C2a, C3a, C4a, C5a, C1, C2, C3, C4, and C5 as defined hereinafter.

In another embodiment, R^{r} is selected from Group R, and Group R consists of

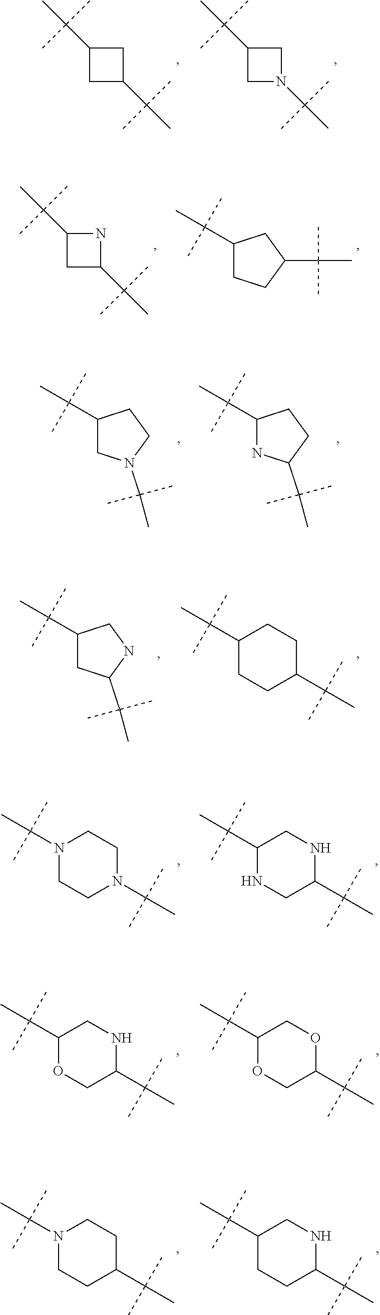

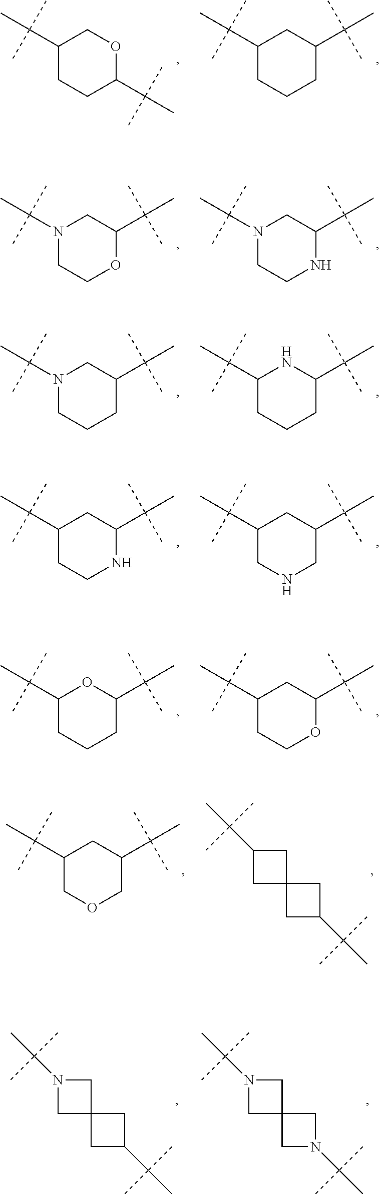

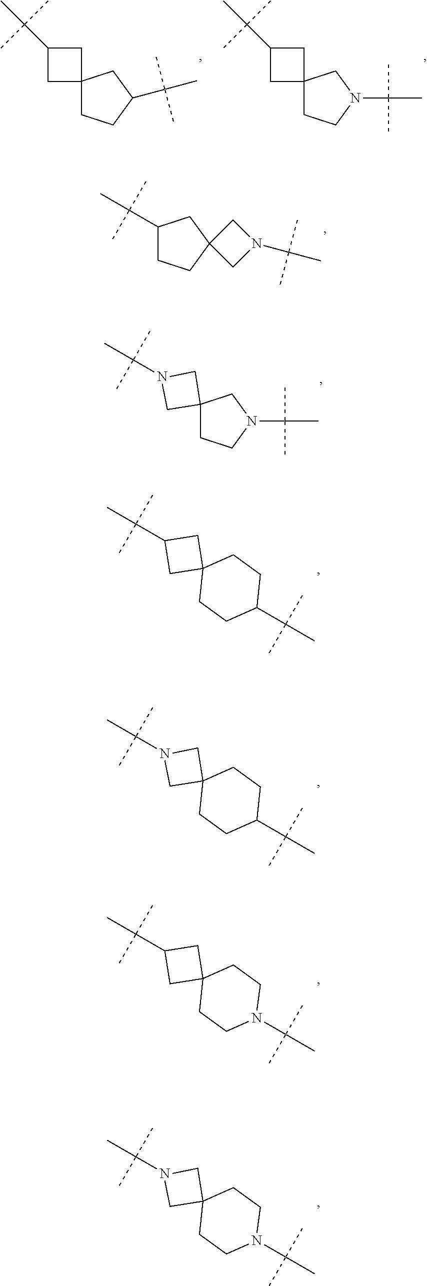

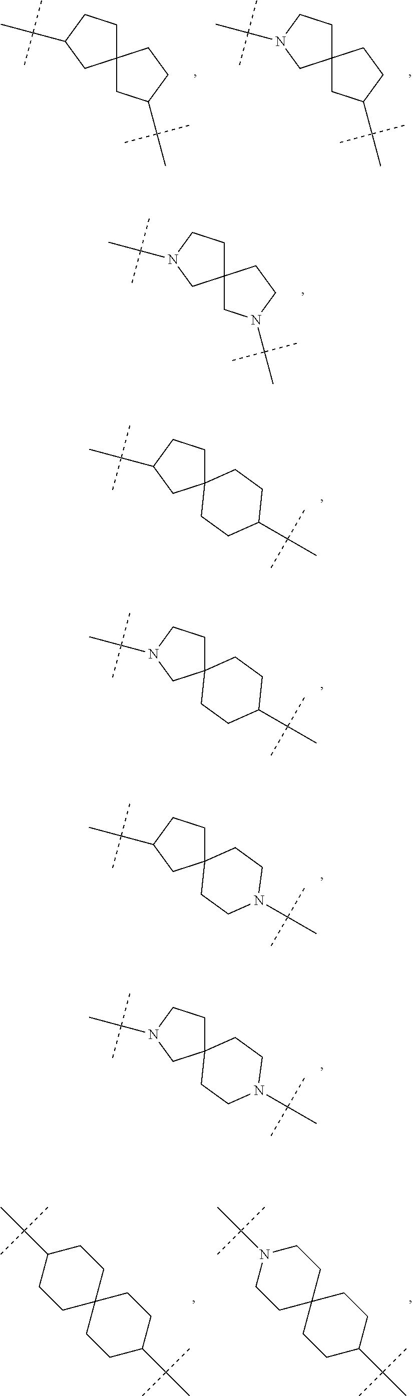

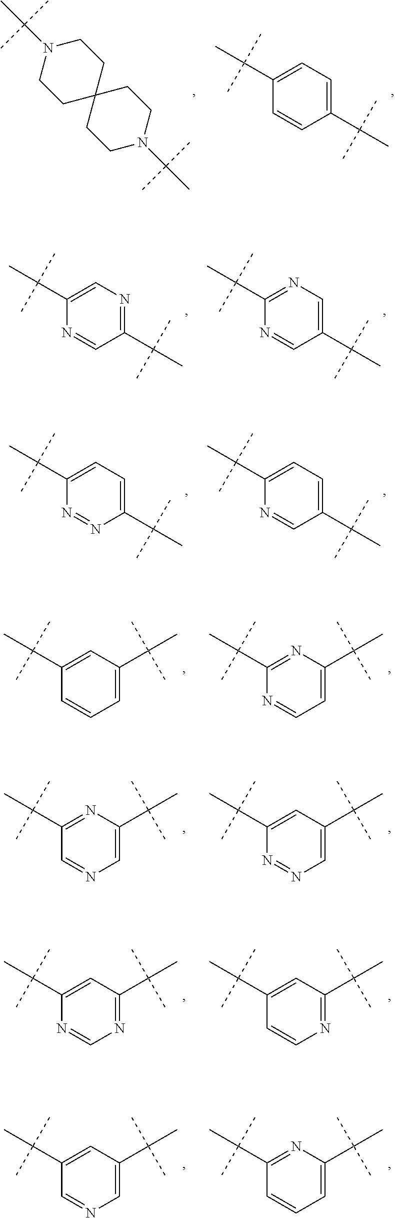

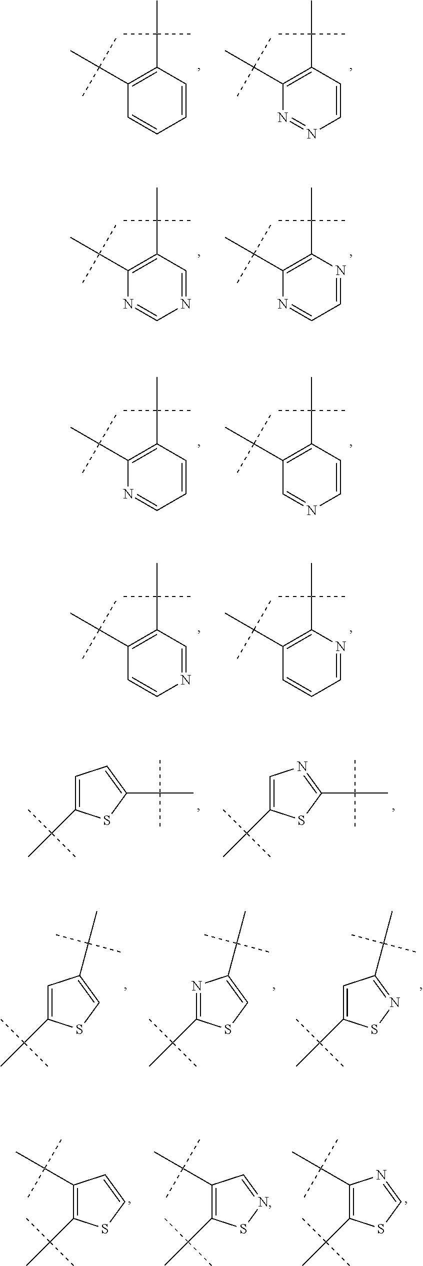

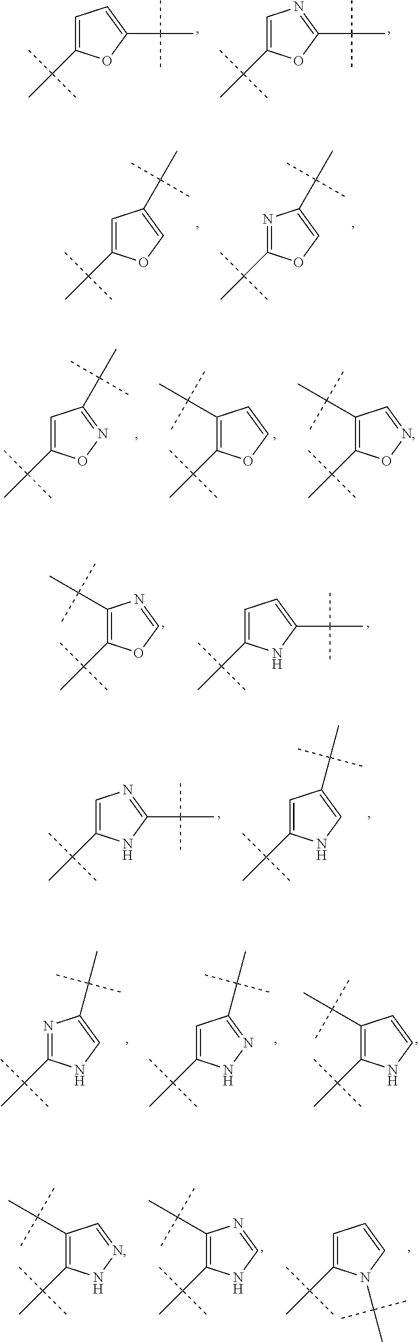

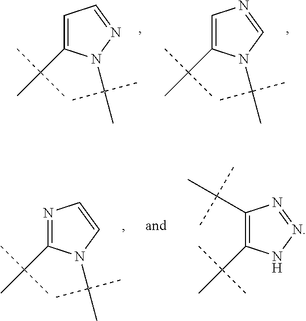

In one embodiment, the linker moiety is of FORMULA 9A:

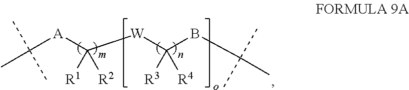

wherein

R¹, R², R³ and R⁴, at each occurrence, are independently selected from hydrogen, halogen, hydroxyl, amino, cyano, nitro, optionally substituted C₁-C₈ alkyl, optionally substituted C₂-C₈ alkenyl, optionally substituted C₂-C₈ alkynyl, optionally substituted C₁-C₈ alkoxy, optionally substituted C₁-C₈ alkoxyalkyl, optionally substituted C₁-C₈ haloalkyl, optionally substituted C₁-C₈ hydroxyalkyl, optionally substituted C₁-C₈ alkylamino, and optionally substituted C₁-C₈ alkylaminoC₁-C₈ alkyl, optionally substituted 3-10 membered carbocyclyl, optionally substituted 3-8 membered cycloalkoxy, optionally substituted 3-10 membered carbocyclylamino, optionally substituted 4-8 membered heterocyclyl, optionally substituted aryl, and optionally substituted heteroaryl, or

R¹ and R², R³ and R⁴ together with the atom to which they are connected form a 3-20 membered cycloalkyl or 4-20 membered heterocyclyl ring;

A, W and B, at each occurrence, are independently selected from null, or bivalent moiety selected from R′-R″, R′COR″, R′CO₂R″, R′C(O)N(R⁵)R″, R′C(S)N(R⁵)R″, R′OR″, R′SR″, R′SOR″, R′SO₂R″, R′SO₂N(R⁵)R″, R′N(R⁵)R″, R′N(R⁵)COR″, R′N(R⁵)CON(R⁶)R″, R′N(R⁵)C(S)R″, optionally substituted C₁-C₈ alkylene, optionally substituted C₂-C₈ alkenylene, optionally substituted C₂-C₈ alkynylene, optionally substituted C₁-C₈alkoxyC₁-C₈alkylene, optionally substituted C₁-C₈ haloalkylene, optionally substituted C₁-C₈ hydroxyalkylene, optionally substituted C₃-C₁₃ fused cycloalkyl, optionally substituted C₃-C₁₃ fused heterocyclyl, optionally substituted C₃-C₁₃ bridged cycloalkyl, optionally substituted C₃-C₁₃ bridged heterocyclyl, optionally substituted C₃-C₁₃ spiro cycloalkyl, optionally substituted C₃-C₁₃ spiro heterocyclyl, optionally substituted 3-10 membered carbocyclyl, optionally substituted 4-10 membered heterocyclyl, optionally substituted aryl, and optionally substituted heteroaryl, wherein

R′ and R″ are independently selected from null, optionally substituted (C₁-C₈ alkyl)-R^{r} (preferably, CH₂-R^{r}), optionally substituted R^{r}—(C₁-C₈ alkylene), optionally substituted (C₁-C₈ alkylene)-R^{r}—(C₁-C₈ alkylene), or a moiety comprising of optionally substituted C₁-C₈ alkyl, optionally substituted C₂-C₈ alkenyl, optionally substituted C₂-C₈ alkynyl, optionally substituted C₁-C₈ hydroxyalkyl, optionally substituted C₁-C₈alkoxyC₁-C₈alkyl, optionally substituted C₁-C₈alkylaminoC₁-C₈alkyl, optionally substituted C₁-C₈ haloalkyl, optionally substituted C₁-C₈ alkylene, optionally substituted C₂-C₈ alkenylene, optionally substituted C₂-C₈ alkynylene, optionally substituted C₁-C₈ hydroxyalkylene, optionally substituted C₁-C₈alkoxyC₁-C₈alkylene, optionally substituted C₁-C₈alkylaminoC₁-C₈alkylene, optionally substituted C₁-C₈ haloalkylene, optionally substituted 3-10 membered carbocyclyl, optionally substituted 4-10 membered heterocyclyl, optionally substituted C₃-C₁₃ fused cycloalkyl, optionally substituted C₃-C₁₃ fused heterocyclyl, optionally substituted C₃-C₁₃ bridged cycloalkyl, optionally substituted C₃-C₁₃ bridged heterocyclyl, optionally substituted C₃-C₁₃ spiro cycloalkyl, optionally substituted C₃-C₁₃ spiro heterocyclyl, optionally substituted aryl, and optionally substituted heteroaryl;

R^{r} is selected from optionally substituted 3-10 membered carbocyclyl, optionally substituted 4-10 membered heterocyclyl, optionally substituted C₃-C₁₃ fused cycloalkyl, optionally substituted C₃-C₁₃ fused heterocyclyl, optionally substituted C₃-C₁₃ bridged cycloalkyl, optionally substituted C₃-C₁₃ bridged heterocyclyl, optionally substituted C₃-C₁₃ spiro cycloalkyl, optionally substituted C₃-C₁₃ spiro heterocyclyl, optionally substituted aryl, and optionally substituted heteroaryl;

R⁵ and R⁶ are independently selected from hydrogen, optionally substituted C₁-C₈ alkyl, optionally substituted C₂-C₈ alkenyl, optionally substituted C₂-C₈ alkynyl, optionally substituted C₁-C₈ alkoxyalkyl, optionally substituted C₁-C₈ haloalkyl, optionally substituted C₁-C₈ hydroxyalkyl, optionally substituted C₁-C₈alkylaminoC₁-C₈alkyl, optionally substituted 3-10 membered carbocyclyl, optionally substituted 4-10 membered heterocyclyl, optionally substituted aryl, and optionally substituted heteroaryl;

R′ and R″, R⁵ and R⁶, R′ and R⁵, R′ and R⁶, R″ and R⁵, R″ and R⁶ together with the atom to which they are connected form a 3-20 membered cycloalkyl or 4-20 membered heterocyclyl ring;

m is 0 to 15;

n, at each occurrence, is 0 to 15; and

o is 0 to 15.

In another embodiment, A, W and B, at each occurrence, are independently selected from null, CO, NH, NH—CO, CO—NH, —(CH₂)₀₋₈—, —(CH₂)₀₋₃—CO—(CH₂)₀₋₈—, (CH₂)₀₋₈—NH—CO, (CH₂)₀₋₈—CO—NH, NH—CO—(CH₂)₀₋₈, CO—NH—(CH₂)₀₋₈, (CH₂)₁₋₃—NH—(CH₂)₁₋₃—CO—NH, (CH₂)₁₋₃—NH—(CH₂)₁₋₃—NH—CO, —CO—NH, CO—NH—(CH₂)₁₋₃—NH—(CH₂)₁₋₃, (CH₂)₁₋₃—NH—(CH₂)₁₋₃, —(CH₂)₀₋₃—R^{r}—(CH₂)₀₋₃, —(CH₂)₀₋₃—(CO)—(CH₂)₀₋₃—R^{r}—(CH₂)₀₋₃—, —(CH₂)₀₋₃—(CO—NH)—(CH₂)₀₋₃—R^{r}—(CH₂)₀₋₃—, —(CH₂)₀₋₃—(NH—CO)—(CH₂)₀₋₃—R^{r}—(CH₂)₀₋₃—, and —(CH₂)₀₋₃—(NH)—(CH₂)₀₋₃—R^{r}—(CH₂)₀₋₃—.

In another embodiment, W and m are defined as above; and A and B, at each occurrence, are independently selected from null, CO, NH, NH—CO, CO—NH, —(CH₂)₀₋₈—, —(CH₂)₀₋₃—CO—(CH₂)₀₋₈—, (CH₂)₁₋₂—NH—CO, (CH₂)₁₋₂—CO—NH, NH—CO—(CH₂)₁₋₂, CO—NH—(CH₂)₁₋₂, (CH₂)₁₋₂—NH—(CH₂)₁₋₂—CO—NH, (CH₂)₁₋₂—NH—(CH₂)₁₋₂—NH—CO, —CO—NH, CO—NH—(CH₂)₁₋₂—NH—(CH₂)₁₋₂, (CH₂)₁₋₂—NH—(CH₂)₁₋₂, —(CH₂)₀₋₂—R^{r}—(CH₂)₀₋₂, —(CH₂)₀₋₂—(CO)—(CH₂)₀₋₃—Rᵣ—(CH₂)₀₋₂—, —(CH₂)₀₋₂—(CO—NH)—(CH₂)₀₋₃—R^{r}—(CH₂)₀₋₂—, —(CH₂)₀₋₂—(NH—CO)—(CH₂)₀₋₃—Rᵣ—CH₂)₀₋₂—, —(CH₂)₀₋₂—(NH)—(CH₂)₀₋₃—R^{r}—(CH₂)₀₋₂—In another embodiment, R^{r} is selected from FORMULA C1a, C2a, C3a, C4a, C5a, C1, C2, C3, C4, and C5 as defined hereinafter.

In another embodiment, R^{r} is selected from Group R, and Group R is defined as in FORMULA 9.

In one embodiment, the CBP/P300 ligand of the bivalent compound is attached to A in FORMULA 9A.

In another embodiment, A (when A is attached to the CBP/P300 ligand) is selected from null, CO, NH, NH—CO, CO—NH, —(CH₂)₀₋₈—, —(CH₂)₀₋₃—CO—(CH₂)₀₋₈—, (CH₂)₀₋₈—NH—CO, (CH₂)₀₋₈—CO—NH, NH—CO—(CH₂)₀₋₈, CO—NH—(CH₂)₀₋₈, (CH₂)₁₋₃—NH—(CH₂)₁₋₃—CO—NH, (CH₂)₁₋₃—NH—(CH₂)₁₋₃—NH—CO, CO—NH—(CH₂)₁₋₃—NH—(CH₂)₁₋₃, (CH₂)₁₋₃—NH—(CH₂)₁₋₃, —(CH₂)₀₋₃—R^{r}—(CH₂)₀₋₃, —(CH₂)₀₋₃—(CO)—(CH₂)₀₋₃—R^{r}—(CH₂)₀₋₃—, —(CH₂)₀₋₃—(CO—NH)—(CH₂)₀₋₃—R^{r}—(CH₂)₀₋₃—, —(CH₂)₀₋₃—(NH—CO)—(CH₂)₀₋₃—R^{r}—(CH₂)₀₋₃—, and —(CH₂)₀₋₃—(NH)—(CH₂)₀₋₃—R^{r}—(CH₂)₀₋₃—, wherein

Rᵣ is selected from Group R, and Group R is defined as in FORMULA 9; and,

W and B is null.

In one embodiment, the linker moiety is of FORMULA 9A:

wherein

R¹, R², R³ and R⁴, at each occurrence, are independently selected from hydrogen, optionally substituted C₁-C₈ alkyl (preferably, C₁-C₄ alkyl), or

R¹ and R², R³ and R⁴ together with the atom to which they are connected form a 3-20 membered cycloalkyl (preferably, 3-5 membered cycloalkyl) or 4-20 membered heterocyclyl ring;

A is defined as before; and W and B are null;

m is 0 to 15 (preferably, m is 0, 1, or 2);

n, at each occurrence, is 1 to 15 (preferably, n is 1); and

o is 1 to 15 (preferably, o is 1, 2, 3, 4, 5, 6, 7, 8, 9, 10, 11, 12 or 13).

In another embodiment, A is independently selected from null, or bivalent moiety selected from R′-R″, R′COR″, R′CO₂R″, R′C(O)N(R⁵)R″, R′C(S)N(R⁵)R″, R′OR″, R′SR″, R′SOR″, R′SO₂R″, R′SO₂N(R⁵)R″, R′N(R⁵)R″, R′N(R⁵)COR″, R′N(R⁵)CON(R⁶)R″, R′N(R⁵)C(S)R″; R and R″ are defined as above.

In another embodiment, R′ and R″ are independently selected from null, optionally substituted (C₁-C₈ alkylene)-R^{r} (preferably, CH₂—R^{r}), or optionally substituted C₁-C₈ alkyl (preferably, optionally substituted C₁-C₂ alkyl).

In another embodiment, the linker moiety is of FORMULA 9B:

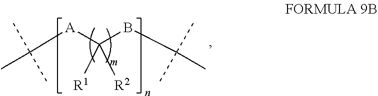

wherein

R¹ and R², at each occurrence, are independently selected from hydrogen, halogen, hydroxyl, amino, cyano, nitro, and optionally substituted C₁-C₈ alkyl, optionally substituted C₁-C₈ alkoxy, optionally substituted C₁-C₈ alkoxy C₁-C₈ alkyl, optionally substituted C₁-C₈ haloalkyl, optionally substituted C₁-C₈ hydroxyalkyl, optionally substituted C₁-C₈ alkylamino, C₁-C₈alkylaminoC₁-C₈alkyl, optionally substituted 3-10 membered carbocyclyl, optionally substituted 3-8 membered cycloalkoxy, optionally substituted 3-10 membered carbocyclylamino, optionally substituted 4-10 membered heterocyclyl, optionally substituted aryl, and optionally substituted heteroaryl, or

R¹ and R² together with the atom to which they are connected form a 3-20 membered cycloalkyl or 4-20 membered heterocyclyl ring;

A and B, at each occurrence, are independently selected from null, or bivalent moiety selected from R′-R″, R′COR″, R′CO₂R″, R′C(O)N(R³)R″, R′C(S)N(R³)R″, R′OR″, R′SR″, R′SOR″, R′SO₂R″, R′SO₂N(R³)R″, R′N(R³)R″, R′N(R³)COR″, R′N(R³)CON(R⁴)R″, R′N(R³)C(S)R″, optionally substituted C₁-C₈ alkylene, optionally substituted C₂-C₈ alkenylene, optionally substituted C₂-C₈ alkynylene, optionally substituted C₁-C₈alkoxyC₁-C₈alkylene, optionally substituted C₁-C₈ haloalkylene, optionally substituted C₁-C₈ hydroxyalkylene, optionally substituted C₃-C₁₃ fused cycloalkyl, optionally substituted C₃-C₁₃ fused heterocyclyl, optionally substituted C₃-C₁₃ bridged cycloalkyl, optionally substituted C₃-C₁₃ bridged heterocyclyl, optionally substituted C₃-C₁₃ spiro cycloalkyl, optionally substituted C₃-C₁₃ spiro heterocyclyl, optionally substituted 3-10 membered carbocyclyl, optionally substituted 4-10 membered heterocyclyl, optionally substituted aryl, and optionally substituted heteroaryl, wherein

R′ and R″ are independently selected from null, optionally substituted (C₁-C₈ alkylene)-R^{r} (preferably, CH₂-R^{r}), optionally substituted R^{r}—(C₁-C₈ alkylene), optionally substituted (C₁-C₈ alkylene)-R^{r}—(C₁-C₈ alkylene), or a moiety comprising of optionally substituted C₁-C₈ alkyl, optionally substituted C₂-C₈ alkenyl, optionally substituted C₂-C₈ alkynyl, optionally substituted C₁-C₈ hydroxyalkyl, optionally substituted C₁-C₈alkoxyC₁-C₈alkyl, optionally substituted C₁-C₈alkylaminoC₁-C₈alkyl, optionally substituted C₁-C₈ haloalkyl, optionally substituted C₁-C₈ alkylene, optionally substituted C₂-C₈ alkenylene, optionally substituted C₂-C₈ alkynylene, optionally substituted C₁-C₈ hydroxyalkylene, optionally substituted C₁-C₈alkoxyC₁-C₈alkylene, optionally substituted C₁-C₈alkylaminoC₁-C₈alkylene, optionally substituted C₁-C₈ haloalkylene, optionally substituted 3-10 membered carbocyclyl, optionally substituted 4-10 membered heterocyclyl, optionally substituted C₃-C₁₃ fused cycloalkyl, optionally substituted C₃-C₁₃ fused heterocyclyl, optionally substituted C₃-C₁₃ bridged cycloalkyl, optionally substituted C₃-C₁₃ bridged heterocyclyl, optionally substituted C₃-C₁₃ spiro cycloalkyl, optionally substituted C₃-C₁₃ spiro heterocyclyl, optionally substituted aryl, and optionally substituted heteroaryl;

R^{r} is selected from optionally substituted 3-10 membered carbocyclyl, optionally substituted 4-10 membered heterocyclyl, optionally substituted C₃-C₁₃ fused cycloalkyl, optionally substituted C₃-C₁₃ fused heterocyclyl, optionally substituted C₃-C₁₃ bridged cycloalkyl, optionally substituted C₃-C₁₃ bridged heterocyclyl, optionally substituted C₃-C₁₃ spiro cycloalkyl, optionally substituted C₃-C₁₃ spiro heterocyclyl, optionally substituted aryl, and optionally substituted heteroaryl;

R³ and R⁴ are independently selected from hydrogen, optionally substituted C₁-C₈ alkyl, optionally substituted C₂-C₈ alkenyl, optionally substituted C₂-C₈ alkynyl, optionally substituted C₁-C₈ alkoxyalkyl, optionally substituted C₁-C₈ haloalkyl, optionally substituted C₁-C₈ hydroxyalkyl, optionally substituted C₁-C₈alkylaminoC₁-C₈alkyl, optionally substituted 3-10 membered carbocyclyl, optionally substituted 4-10 membered heterocyclyl, optionally substituted aryl, and optionally substituted heteroaryl;

R′ and R″, R³ and R⁴, R′ and R³, R′ and R⁴, R″ and R³, R″ and R⁴ together with the atom to which they are connected form a 3-20 membered cycloalkyl or 4-20 membered heterocyclyl ring;

each m is 0 to 15; and

n is 0 to 15.

In another embodiment, A, W and B, at each occurrence, are independently selected from null, CO, NH, NH—CO, CO—NH, —(CH₂)₀₋₈—, —(CH₂)₀₋₃—CO—(CH₂)₀₋₁₀—, (CH₂)₀₋₈—NH—CO, (CH₂)₀₋₈—CO—NH, NH—CO—(CH₂)₀₋₈, CO—NH—(CH₂)₀₋₈, (CH₂)₁₋₃—NH—(CH₂)₁₋₃—CO—NH, (CH₂)₁₋₃—NH—(CH₂)₁₋₃—NH—CO, CO—NH—(CH₂)₁₋₃—NH—(CH₂)₁₋₃, (CH₂)₁₋₃—NH—(CH₂)₁₋₃, —(CH₂)₀₋₃—R^{r}—(CH₂)₀₋₃, —(CH₂)₀₋₃—(CO)—(CH₂)₀₋₃—R^{r}—(CH₂)₀₋₃—, —(CH₂)₀₋₃—(CO—NH)—(CH₂)₀₋₃—R^{r}—(CH₂)₀₋₃—, —(CH₂)₀₋₃—(NH—CO)—(CH₂)₀₋₃—R^{r}—(CH₂)₀₋₃—, —(CH₂)₀₋₃—(NH)—(CH₂)₀₋₃—R^{r}—(CH₂)₀₋₃—.

In another embodiment, R^{r} is selected from FORMULA C1a, C2a, C3a, C4a, C5a, C1, C2, C3, C4, and C5 as defined hereinafter.

In another embodiment, Rᵣ is selected from Group R, and Group R is defined as in FORMULA 9.

In another embodiment, the linker moiety is of FORMULA 9C:

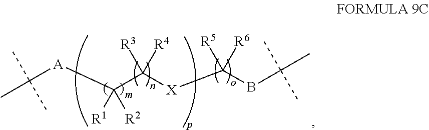

wherein

X is selected from O, NH, and NR⁷;

R¹, R², R³, R⁴, R⁵, and R⁶, at each occurrence, are independently selected from hydrogen, halogen, hydroxyl, amino, cyano, nitro, optionally substituted C₁-C₈ alkyl, optionally substituted C₂-C₈ alkenyl, optionally substituted C₂-C₈ alkynyl, optionally substituted C₁-C₈ alkoxy, optionally substituted C₁-C₈ alkoxy C₁-C₈ alkyl, optionally substituted C₁-C₈ haloalkyl, optionally substituted C₁-C₈ hydroxyalkyl, optionally substituted C₁-C₈ alkylamino, optionally substituted C₁-C₈ alkylaminoC₁-C₈ alkyl, optionally substituted 3-10 membered carbocyclyl, optionally substituted 3-8 membered cycloalkoxy, optionally substituted 4-10 membered heterocyclyl, optionally substituted aryl, and optionally substituted heteroaryl;

A and B are independently selected from null, or bivalent moiety selected from R′-R″, R′COR″, R′CO₂R″, R′C(O)N(R⁸)R″, R′C(S)N(R⁸)R″, R′OR″, R′SR″, R′SOR″, R′SO₂R″, R′SO₂N(R⁸)R″, R′N(R⁸)R″, R′N(R⁸)COR″, R′N(R⁸)CON(R⁹)R″, R′N(R⁸)C(S)R″, optionally substituted C₁-C₈ alkylene, optionally substituted C₂-C₈ alkenylene, optionally substituted C₂-C₈ alkynylene, optionally substituted C₁-C₈alkoxyC₁-C₈alkylene, optionally substituted C₁-C₈ haloalkylene, optionally substituted C₁-C₈ hydroxyalkylene, optionally substituted C₃-C₁₃ fused cycloalkyl, optionally substituted C₃-C₁₃ fused heterocyclyl, optionally substituted C₃-C₁₃ bridged cycloalkyl, optionally substituted C₃-C₁₃ bridged heterocyclyl, optionally substituted C₃-C₁₃ spiro cycloalkyl, optionally substituted C₃-C₁₃ spiro heterocyclyl, optionally substituted 3-10 membered carbocyclyl, optionally substituted 4-10 membered heterocyclyl, optionally substituted aryl, and optionally substituted heteroaryl, wherein

R′ and R″ are independently selected from null, optionally substituted (C₁-C₈ alkylene)-R^{r} (preferably, CH₂-R^{r}), optionally substituted R^{r}—(C₁-C₈ alkylene), optionally substituted (C₁-C₈ alkylene)-R^{r}—(C₁-C₈ alkylene), or a moiety comprising of optionally substituted C₁-C₈ alkyl, optionally substituted C₂-C₈ alkenyl, optionally substituted C₂-C₈ alkynyl, optionally substituted C₁-C₈ hydroxyalkyl, optionally substituted C₁-C₈alkoxyC₁-C₈alkyl, optionally substituted C₁-C₈alkylaminoC₁-C₈alkyl, optionally substituted C₁-C₈ haloalkyl, optionally substituted C₁-C₈ alkylene, optionally substituted C₂-C₈ alkenylene, optionally substituted C₂-C₈ alkynylene, optionally substituted C₁-C₈ hydroxyalkylene, optionally substituted C₁-C₈alkoxyC₁-C₈alkylene, optionally substituted C₁-C₈alkylaminoC₁-C₈alkylene, optionally substituted C₁-C₈ haloalkylene, optionally substituted 3-10 membered carbocyclyl, optionally substituted 4-10 membered heterocyclyl, optionally substituted C₃-C₁₃ fused cycloalkyl, optionally substituted C₃-C₁₃ fused heterocyclyl, optionally substituted C₃-C₁₃ bridged cycloalkyl, optionally substituted C₃-C₁₃ bridged heterocyclyl, optionally substituted C₃-C₁₃ spiro cycloalkyl, optionally substituted C₃-C₁₃ spiro heterocyclyl, optionally substituted aryl, and optionally substituted heteroaryl;

R^{r} is selected from optionally substituted 3-10 membered carbocyclyl, optionally substituted 4-10 membered heterocyclyl, optionally substituted C₃-C₁₃ fused cycloalkyl, optionally substituted C₃-C₁₃ fused heterocyclyl, optionally substituted C₃-C₁₃ bridged cycloalkyl, optionally substituted C₃-C₁₃ bridged heterocyclyl, optionally substituted C₃-C₁₃ spiro cycloalkyl, optionally substituted C₃-C₁₃ spiro heterocyclyl, optionally substituted aryl, and optionally substituted heteroaryl;

R⁷, R⁸ and R⁹ are independently selected from hydrogen, optionally substituted C₁-C₈ alkyl, optionally substituted C₂-C₈ alkenyl, optionally substituted C₂-C₈ alkynyl, optionally substituted C₁-C₈ alkoxyalkyl, optionally substituted C₁-C₈ haloalkyl, optionally substituted C₁-C₈ hydroxyalkyl, optionally substituted C₁-C₈alkylaminoC₁-C₈alkyl, optionally substituted 3-10 membered carbocyclyl, optionally substituted 4-10 membered heterocyclyl, optionally substituted aryl, and optionally substituted heteroaryl;

R′ and R″, R⁸ and R⁹, R′ and R⁸, R′ and R⁹, R″ and R⁸, R″ and R⁹ together with the atom to which they are connected form a 3-20 membered cycloalkyl or 4-20 membered heterocyclyl ring;

m, at each occurrence, is 0 to 15;

n, at each occurrence, is 0 to 15;

o is 0 to 15; and

p is 0 to 15.

In another embodiment, A and B are independently selected from null, CO, NH, NH—CO, CO—NH, —(CH₂)₀₋₈—, —(CH₂)₀₋₃—CO—(CH₂)₀₋₈—, (CH₂)₀₋₈—NH—CO, (CH₂)₀₋₈—CO—NH, NH—CO—(CH₂)₀₋₈, CO—NH—(CH₂)₀₋₈, (CH₂)₁₋₃—NH—(CH₂)₁₋₃—CO—NH, (CH₂)₁₋₃—NH—(CH₂)₁₋₃—NH—CO, —CO—NH, CO—NH—(CH₂)₁₋₃—NH—(CH₂)₁₋₃, (CH₂)₁₋₃—NH—(CH₂)₁₋₃, —(CH₂)₀₋₃—R^{r}—(CH₂)₀₋₃, —(CH₂)₀₋₃—(CO)—(CH₂)₀₋₃—R^{r}—(CH₂)₀₋₃—, —(CH₂)₀₋₃—(CO—NH)—(CH₂)₀₋₃—R^{r}—(CH₂)₀₋₃—, —(CH₂)₀₋₃—(NH—CO)—(CH₂)₀₋₃—R^{r}—(CH₂)₀₋₃—, and —(CH₂)₀₋₃—(NH)—(CH₂)₀₋₃—R^{r}—(CH₂)₀₋₃—.

In another embodiment, R^{r} is selected from Group R, and Group R is defined as in FORMULA 9.

In another embodiment, A and B, at each occurrence, are independently selected from null, CO, NH, NH—CO, CO—NH, CH₂—NH—CO, CH₂—CO—NH, NH—CO—CH₂, CO—NH—CH₂, CH₂—NH—CH₂—CO—NH, CH₂—NH—CH₂—NH—CO, —CO—NH, CO—NH—CH₂—NH—CH₂, CH₂—NH—CH₂,

In another embodiment, o is 0 to 5.

In another embodiment, the linker moiety comprises one or more rings selected from the group consisting of 3 to 13 membered rings, 3 to 13 membered fused rings, 3 to 13 membered bridged rings, and 3 to 13 membered spiro rings.

In another embodiment, the linker moiety comprises one or more rings selected from the group consisting of formulae C1a, C2a, C3a, C4a and C5a:

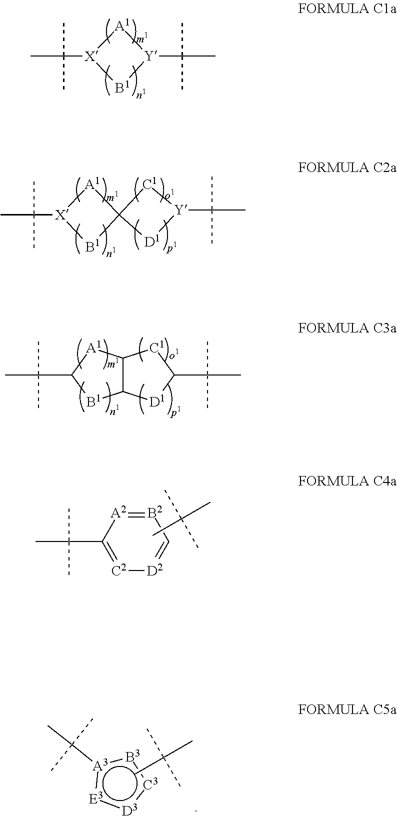

wherein

X′ and Y′ are independently selected from N, CR^{b};

A¹, B¹, C¹ and D¹, at each occurrence, are independently selected from null, O, CO, SO, SO₂, NR^{b}, CR^{b}R^{c};

A², B², C², and D², at each occurrence, are independently selected from N, CR^{b};

A³, B³, C³, D³, and E³, at each occurrence, are independently selected from N, O, S, NR^{b}, CR^{b};

R^{b} and R^{c}, at each occurrence, are independently selected from hydrogen, halogen, hydroxyl, amino, cyano, nitro, optionally substituted C₁-C₈ alkyl, optionally substituted C₂-C₈ alkenyl, optionally substituted C₂-C₈ alkynyl, optionally substituted C₁-C₈ alkoxy, optionally substituted C₁-C₈ alkoxyalkyl, optionally substituted C₁-C₈ haloalkyl, optionally substituted C₁-C₈ hydroxyalkyl, optionally substituted C₁-C₈ alkylamino, and optionally substituted C₁-C₈ alkylaminoC₁-C₈ alkyl, optionally substituted 3-10 membered carbocyclyl, optionally substituted 3-8 membered cycloalkoxy, optionally substituted 3-10 membered carbocyclylamino, optionally substituted 4-8 membered heterocyclyl, optionally substituted aryl, and optionally substituted heteroaryl; and

m¹, n¹, o¹ and p¹ are independently selected from 0, 1, 2, 3, 4 and 5.

In another embodiment, the linker moiety comprises one or more rings selected from the group consisting of formulae C1, C2, C3, C4 and C5:

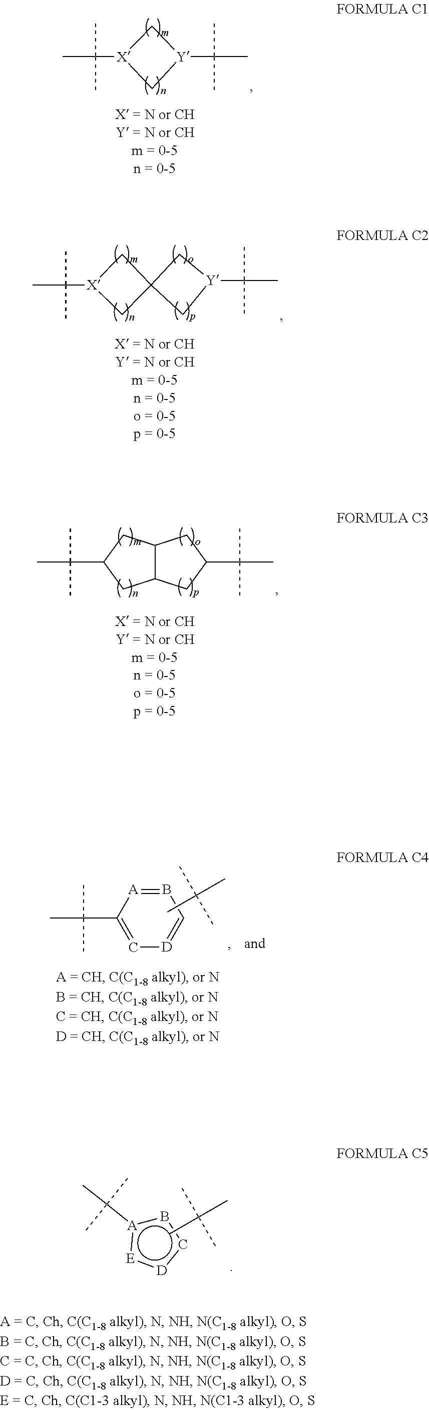

In another embodiment, the linker moiety is of FORMULA 9A.

In another embodiment, A, W and B, at each occurrence, are independently selected from null, CO, NH, NH—CO, CO—NH, —(CH₂)₀₋₈—, —(CH₂)₀₋₃—CO—(CH₂)₀₋₈—, (CH₂)₀₋₈—NH—CO, (CH₂)₀₋₈—CO—NH—, NH—CO—(CH₂)₀₋₈, CO—NH—(CH₂)₀₋₈, (CH₂)₁₋₃—NH—(CH₂)₁₋₃—CO—NH, (CH₂)₁₋₃—NH—(CH₂)₁₋₃—NH—CO, CO—NH—(CH₂)₁₋₃—NH—(CH₂)₁₋₃, (CH₂)₁₋₃—NH—(CH₂)₁₋₃, —(CH₂)₀₋₃—R^{r}—(CH₂)₀₋₃, —(CH₂)₀₋₃—(CO)—(CH₂)₀₋₃—R^{r}—(CH₂)₀₋₃—, —(CH₂)₀₋₃—(CO—NH)—(CH₂)₀₋₃—R^{r}—(CH₂)₀₋₃—, —(CH₂)₀₋₃—(NH—CO)—(CH₂)₀₋₃—R^{r}—(CH₂)₀₋₃—, and —(CH₂)₀₋₃—(NH)—(CH₂)₀₋₃—R^{r}—(CH₂)₀₋₃—.

In another embodiment, R^{r} is selected from FORMULA C1a, C2a, C3a, C4a, C5a, C1, C2, C3, C4, and C5 as defined above.

In another embodiment, R^{r} is selected from Group R, and Group R is defined as in FORMULA 9.

In another embodiment, in FORMULA 9A, A and B are independently defined as above, and W is null.

In another embodiment, the length of the linker is 0 to 40 chain atoms.

In another embodiment, the length of the linker is 3 to 20 chain atoms.

In another embodiment, the length of the linker is 5-15 chain atoms.

In another embodiment, when the CBP/P300 ligand of the bivalent compound attached to A, A is selected from —(CO)—, —(CH₂)₁₋₂(CO)—NH—, —(CH₂)₀₋₈—, —(CH₂)₀₋₃—CO—(CH₂)₀₋₈—, —(CH₂)₀₋₃—R^{r}—(CH₂)₀₋₃, —(CH₂)₀₋₃—(CO)—(CH₂)₀₋₃—R^{r}—(CH₂)₀₋₃, wherein R^{r} is selected from Group R, and Group R is defined as in FORMULA 9.

In another embodiment, the linker is —(CO)—(CH₂)₃₋₇—.

In another embodiment, the linker is —(CH₂)₁₋₂(CO)—NH—(CH₂)₃₋₇—.

In another embodiment, the linker is —(CH₂)₀₋₁₀—, and —(CH₂)₀₋₃—CO—(CH₂)₀₋₁₀—,

In another embodiment, the linker is —(CH₂)₀₋₃—R^{r}—(CH₂)₀₋₃—, or —(CH₂)₀₋₃—(CO)—(CH₂)₀₋₃—R^{r}—(CH₂)₀₋₃—, wherein R^{r} is selected from the group of Group R, and Group R is defined as in FORMULA 9.

Without wishing to be bound by any particular theory, it is contemplated herein that, in some embodiments, attaching pomalidomide or VHL-1 to either portion of the molecule can recruit the cereblon E3 ligase or VHL E3 ligase to CBP/P300.

The bivalent compounds disclosed herein can selectively affect CBP/P300-mediated disease cells compared to WT (wild type) cells (i.e., an bivalent compound able to kill or inhibit the growth of an CBP/P300-mediated disease cell while also having a relatively low ability to lyse or inhibit the growth of a WT cell), e.g., possess a GI₅₀ for one or more CBP/P300-mediated disease cells more than 1.5-fold lower, more than 2-fold lower, more than 2.5-fold lower, more than 3-fold lower, more than 4-fold lower, more than 5-fold lower, more than 6-fold lower, more than 7-fold lower, more than 8-fold lower, more than 9-fold lower, more than 10-fold lower, more than 15-fold lower, or more than 20-fold lower than its GI₅₀ for one or more WT cells, e.g., WT cells of the same species and tissue type as the CBP/P300-mediated disease cells.

In some aspects, provided herein is a method for identifying a bivalent compound which mediates degradation or reduction of CBP/P300, the method comprising: providing a heterobifunctional test compound comprising an CBP/P300 ligand conjugated to a degradation tag through a linker; contacting the heterobifunctional test compound with a cell comprising a ubiquitin ligase and CBP/P300; determining whether CBP/P300 level is decreased in the cell; and identifying the heterobifunctional test compound as a bivalent compound which mediates degradation or reduction of CBP/P300. In certain embodiments, the cell is a cancer cell. In certain embodiments, the cancer cell is a CBP/P300-mediated cancer cell.

Synthesis and Testing of Bivalent Compounds

The binding affinity of novel synthesized bivalent compounds can be assessed using standard biophysical assays known in the art (e.g., isothermal titration calorimetry (ITC), surface plasmon resonance (SPR)). Cellular assays can then be used to assess the bivalent compound's ability to induce CBP/P300 degradation and inhibit cancer cell proliferation. Besides evaluating a bivalent compound's induced changes in the protein levels of CBP/P300, CBP/P300 mutants, or CBP/P300 fusion proteins, protein-protein interaction or acteryltransferase enzymatic activity can also be assessed. Assays suitable for use in any or all of these steps are known in the art, and include, e.g., western blotting, quantitative mass spectrometry (MS) analysis, flow cytometry, enzymatic activity assay, ITC, SPR, cell growth inhibition, xenograft, orthotopic, and patient-derived xenograft models. Suitable cell lines for use in any or all of these steps are known in the art and include LNCaP, 22RV1, HEL, MV4; 11, RS4; 11, NCI-H929, MM.1S, Pfeiffer, NCI-H520 and other cell lines. Suitable mouse models for use in any or all of these steps are known in the art and include subcutaneous xenograft models, orthotopic models, patient-derived xenograft models, and patient-derived orthotopic models.

By way of non-limiting example, detailed synthesis protocols are described in the Examples for specific exemplary bivalent compounds.

Pharmaceutically acceptable isotopic variations of the compounds disclosed herein are contemplated and can be synthesized using conventional methods known in the art or methods corresponding to those described in the Examples (substituting appropriate reagents with appropriate isotopic variations of those reagents). Specifically, an isotopic variation is a compound in which at least one atom is replaced by an atom having the same atomic number, but an atomic mass different from the atomic mass usually found in nature. Useful isotopes are known in the art and include, for example, isotopes of hydrogen, carbon, nitrogen, oxygen, phosphorus, sulfur, fluorine, and chlorine. Exemplary isotopes thus include, e.g., ²H, ³H, ¹³C, ¹⁴C, ¹⁵N, ¹⁷O, ¹⁸O, ³²P, ³⁵S, ¹⁸R, and ³⁶Cl.

Isotopic variations (e.g., isotopic variations containing ²H) can provide therapeutic advantages resulting from greater metabolic stability, e.g., increased in vivo half-life or reduced dosage requirements. In addition, certain isotopic variations (particularly those containing a radioactive isotope) can be used in drug or substrate tissue distribution studies. The radioactive isotopes tritium (³H) and carbon-14 (¹⁴C) are particularly useful for this purpose in view of their ease of incorporation and ready means of detection.

Pharmaceutically acceptable solvates of the compounds disclosed herein are contemplated. A solvate can be generated, e.g., by substituting a solvent used to crystallize a compound disclosed herein with an isotopic variation (e.g., D₂O in place of H₂O, d₆-acetone in place of acetone, or d₆-DMSO in place of DMSO).

Pharmaceutically acceptable fluorinated variations of the compounds disclosed herein are contemplated and can be synthesized using conventional methods known in the art or methods corresponding to those described in the Examples (substituting appropriate reagents with appropriate fluorinated variations of those reagents). Specifically, a fluorinated variation is a compound in which at least one hydrogen atom is replaced by a fluoro atom. Fluorinated variations can provide therapeutic advantages resulting from greater metabolic stability, e.g., increased in vivo half-life or reduced dosage requirements.

Pharmaceutically acceptable prodrugs of the compounds disclosed herein are contemplated and can be synthesized using conventional methods known in the art or methods corresponding to those described in the Examples (e.g., converting hydroxyl groups or carboxylic acid groups to ester groups). As used herein, a “prodrug” refers to a compound that can be converted via some chemical or physiological process (e.g., enzymatic processes and metabolic hydrolysis) to a therapeutic agent. Thus, the term “prodrug” also refers to a precursor of a biologically active compound that is pharmaceutically acceptable. A prodrug may be inactive when administered to a subject, i.e. an ester, but is converted in vivo to an active compound, for example, by hydrolysis to the free carboxylic acid or free hydroxyl. The prodrug compound often offers advantages of solubility, tissue compatibility or delayed release in an organism. The term “prodrug” is also meant to include any covalently bonded carriers, which release the active compound in vivo when such prodrug is administered to a subject. Prodrugs of an active compound may be prepared by modifying functional groups present in the active compound in such a way that the modifications are cleaved, either in routine manipulation or in vivo, to the parent active compound. Prodrugs include compounds wherein a hydroxy, amino or mercapto group is bonded to any group that, when the prodrug of the active compound is administered to a subject, cleaves to form a free hydroxy, free amino or free mercapto group, respectively. Examples of prodrugs include, but are not limited to, acetate, formate and benzoate derivatives of an alcohol or acetamide, formamide and benzamide derivatives of an amine functional group in the active compound and the like.

Characterization of Exemplary Bivalent Compounds

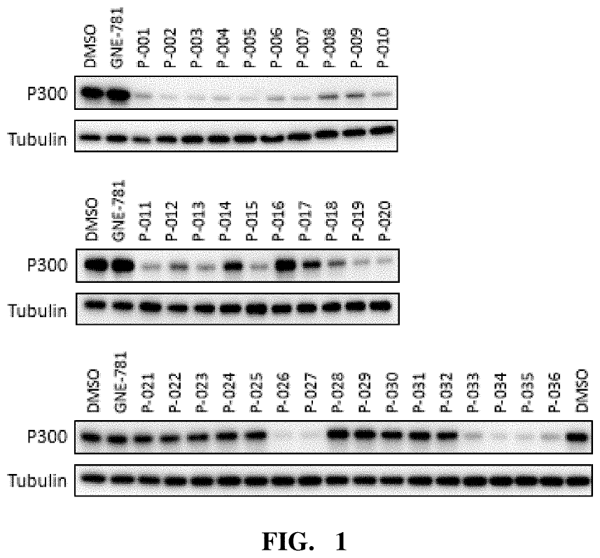

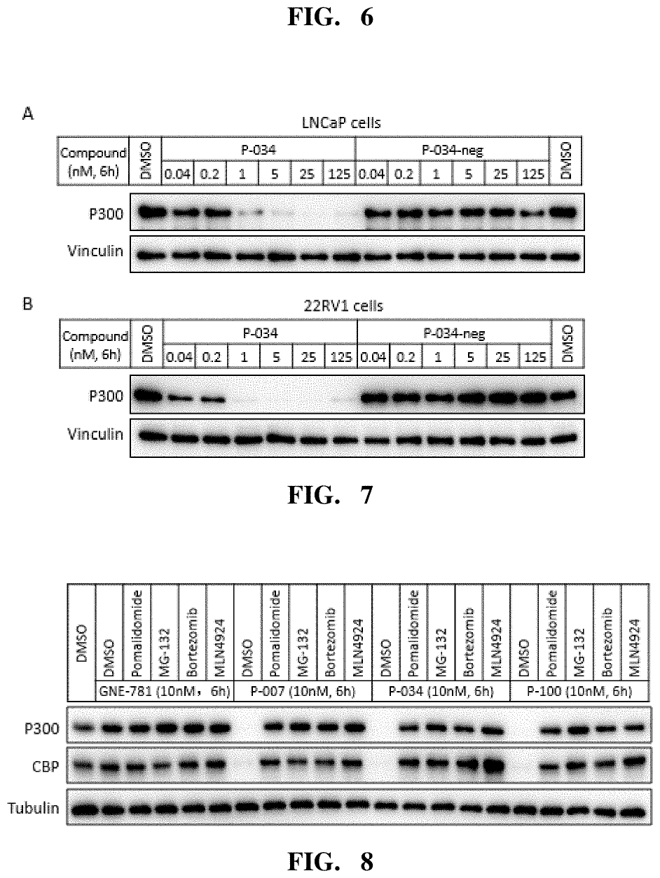

Specific exemplary bivalent compounds were characterized in LNCaP or 22RV1 cells. LNCaP or 22RV1 cells that express CBP/P300 proteins were treated with GNE-781 or the bivalent compounds disclosed herein (P-001 to P-174) for indicated hours. Cells were collected, lysed and subject to immunoblotting using an antibody specific to P300 or CBP proteins. Tubulin or vinculin was included as the loading control. DMSO was used as the negative control. Following treatment of various bivalent compounds, P300 and CBP protein levels in LNCaP or 22RV1 cells were significantly decreased (FIGS. 1, 2, 5, 6, 10, and 11). Selected bivalent compounds disclosed herein were found to be particularly effective in reducing CBP and P300 protein levels, as the concentrations required to reduce target protein levels by 50% (DC₅₀) for some compounds were less than 1 nM (FIG. 6).

In addition, LNCaP cells were treated with 20 nM P-004, P-005, P-015, or P-020 for the indicated time. Subsequently, changes in P300 protein levels were measured via immunoblotting. Tubulin was included as the loading control. Significant degradation of P300 was readily detected as early as 2 hours following administration of the compounds (FIG. 3).

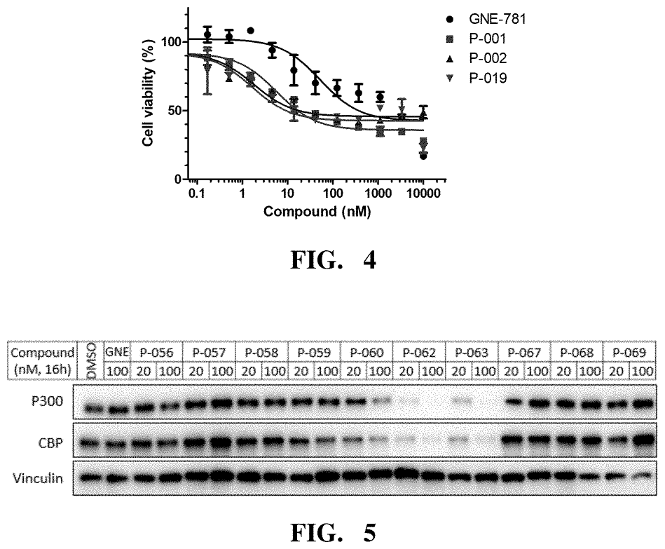

It has been demonstrated that targeting CBP/P300 using ligands to their bromodomains or lysine acetyltransferase domains compromises cancer cell proliferation and survival (Jin et al., 2017; Lasko et al., 2017; Picaud et al., 2015; Popp et al., 2016). LNCaP cells seeded in 96-well plates were treated with 10 μM GNE-781 or selected bivalent compounds, i.e. P-001, P-002, and P-019, following a 12-point 3-fold serial dilution. Three days after treatment, cell viability was determined using the CellTiter-Glo kit (Promega) following manufacturer's instructions. Cell viability was normalized to the mean values of 3 replicates of untreated cells. Dose-dependent response was analyzed following the least-squares non-linear regression method using the GraphPad Prism 5.0 software. Bivalent compounds dose-dependently suppressed viability of LNCaP cells, as exemplified by P-001, P-002, and P-019 (FIG. 4, Table 2-3). These results collectively demonstrated that downregulation of CBP/P300 proteins levels using bivalent compounds described herein induced antineoplastic activities.

The interaction with cereblon is critical to the ability of bivalent compounds to induce degradation of P300/CBP proteins, as a chemical modification that disrupted cereblon binding abolished P300 degradation induced by P-034 in LNCap and 22RV1 cells (FIG. 7). The degradation was also dependent on the ubiquitin-proteasome system, because it could be neutralized by co-administration of proteasome inhibitors, MG-132 and bortezomib, a cullin E3 ligase inhibitor, MLN4924, or high concentration of pomalidomide that compete for cereblon binding, as exemplified by P-007, P-034, and P-100 (FIG. 8).

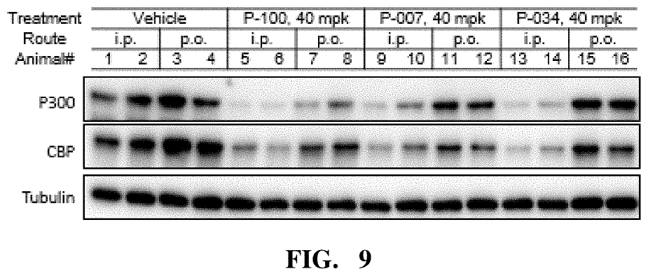

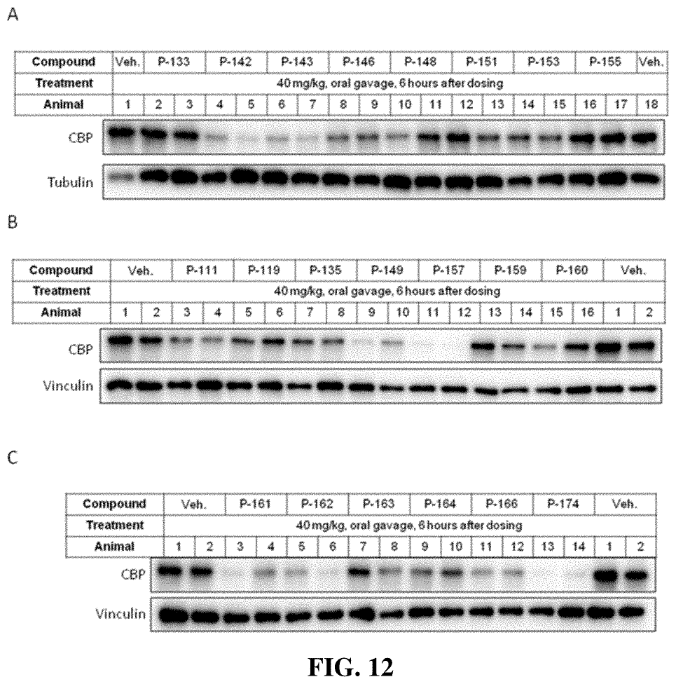

These findings collectively demonstrate that bivalent compounds induce degradation of P300/CBP proteins via a mechanism specifically mediated by cereblon, cullin E3 ligases, and the proteasome. In addition to cultured cells, athymic nude mice bearing 22RV1 subcutaneous xenograft tumors at the right flank were intraperitoneally or orally treated with 40 mg/kg bivalent compounds. Six hours after drug administration, animals were sacrificed for immunoblotting of P300 and CBP in homogenized xenograft tumor masses. Bivalent compounds, as exemplified by P-100, P-007 and P-034, exhibited the ability of significantly reducing P300 and CBP protein levels after a single dose of drug administration (FIG. 9). Moreover, ICR mice were orally treated with 40 mg/kg bivalent compounds. Six hours after drug administration, animals were sacrificed for immunoblotting of CBP in homogenized lung tissues. Bivalent compounds, as exemplified in FIG. 12, exhibited the ability of significantly reducing CBP protein levels after a single dose of drug administration.

### Definition of Terms

As used herein, the terms “comprising” and “including” are used in their open, non-limiting sense.

“Alkyl” refers to a straight or branched hydrocarbon chain radical consisting solely of carbon and hydrogen atoms, containing no unsaturation. An alkyl may comprise one, two, three, four, five, six, seven, eight, nine, ten, eleven, twelve, thirteen, fourteen, fifteen, or sixteen carbon atoms. In certain embodiments, an alkyl comprises one to fifteen carbon atoms (e.g., C₁-C₁₅ alkyl). In certain embodiments, an alkyl comprises one to thirteen carbon atoms (e.g., C₁-C₁₃ alkyl). In certain embodiments, an alkyl comprises one to eight carbon atoms (e.g., C₁-C₈ alkyl). In other embodiments, an alkyl comprises five to fifteen carbon atoms (e.g., C₅-C₁₅ alkyl). In other embodiments, an alkyl comprises five to eight carbon atoms (e.g., C₅-C₈ alkyl). The alkyl is attached to the rest of the molecule by a single bond, for example, methyl (Me), ethyl (Et), n-propyl, 1-methylethyl (iso-propyl), n-butyl, n-pentyl, 1,1-dimethylethyl (t-butyl), pentyl, 3-methylhexyl, 2-methylhexyl, and the like.

“Alkenyl” refers to a straight or branched hydrocarbon chain radical group consisting solely of carbon and hydrogen atoms, containing at least one double bond. An alkenyl may comprise two, three, four, five, six, seven, eight, nine, ten, eleven, twelve, thirteen, fourteen, fifteen, or sixteen carbon atoms. In certain embodiments, an alkenyl comprises two to twelve carbon atoms (e.g., C₂-C₁₂ alkenyl). In certain embodiments, an alkenyl comprises two to eight carbon atoms (e.g., C₂-C₈ alkenyl). In certain embodiments, an alkenyl comprises two to six carbon atoms (e.g., C₂-C₆ alkenyl). In other embodiments, an alkenyl comprises two to four carbon atoms (e.g., C₂-C₄ alkenyl). The alkenyl is attached to the rest of the molecule by a single bond, for example, ethenyl (i.e., vinyl), prop-1-enyl (i.e., allyl), but-1-enyl, pent-1-enyl, penta-1,4-dienyl, and the like.

The term “allyl,” as used herein, means a —CH₂CH═CH₂ group.

As used herein, the term “alkynyl” refers to a straight or branched hydrocarbon chain radical group consisting solely of carbon and hydrogen atoms, containing at least one triple bond. An alkynyl may comprise two, three, four, five, six, seven, eight, nine, ten, eleven, twelve, thirteen, fourteen, fifteen, or sixteen carbon atoms. In certain embodiments, an alkynyl comprises two to twelve carbon atoms (e.g., C₂-C₁₂ alkynyl). In certain embodiments, an alkynyl comprises two to eight carbon atoms (e.g., C₂-C₈ alkynyl). In other embodiments, an alkynyl has two to six carbon atoms (e.g., C₂-C₆ alkynyl). In other embodiments, an alkynyl has two to four carbon atoms (e.g., C₂-C₄ alkynyl). The alkynyl is attached to the rest of the molecule by a single bond. Examples of such groups include, but are not limited to, ethynyl, propynyl, 1-butynyl, 2-butynyl, 1-pentynyl, 2-pentynyl, 1-hexynyl, 2-hexynyl, 3-hexynyl, and the like.

The term “alkoxy”, as used herein, means an alkyl group as defined herein which is attached to the rest of the molecule via an oxygen atom. Examples of such groups include, but are not limited to, methoxy, ethoxy, n-propyloxy, iso-propyloxy, n-butoxy, iso-butoxy, tert-butoxy, pentyloxy, hexyloxy, and the like.

The term “aryl”, as used herein, “refers to a radical derived from an aromatic monocyclic or multicyclic hydrocarbon ring system by removing a hydrogen atom from a ring carbon atom. The aromatic monocyclic or multicyclic hydrocarbon ring system contains only hydrogen and carbon atoms. An aryl may comprise from six to eighteen carbon atoms, where at least one of the rings in the ring system is fully unsaturated, i.e., it contains a cyclic, delocalized (4n+2)π-electron system in accordance with the Hückel theory. In certain embodiments, an aryl comprises six to fourteen carbon atoms (C₆-C₁₄ aryl). In certain embodiments, an aryl comprises six to ten carbon atoms (C₆-C₁₀ aryl). Examples of such groups include, but are not limited to, phenyl, fluorenyl and naphthyl. The terms “Ph” and “phenyl,” as used herein, mean a —C₆H₅ group.

The term “heteroaryl”, refers to a radical derived from a 3- to 18-membered aromatic ring radical that comprises two to seventeen carbon atoms and from one to six heteroatoms selected from nitrogen, oxygen and sulfur. As used herein, the heteroaryl radical may be a monocyclic, bicyclic, tricyclic or tetracyclic ring system, wherein at least one of the rings in the ring system is fully unsaturated, i.e., it contains a cyclic, delocalized (4n+2)π-electron system in accordance with the Hückel theory. In certain embodiments, a heteroaryl refers to a radical derived from a 3- to 10-membered aromatic ring radical (3-10 membered heteroaryl). In certain embodiments, a heteroaryl refers to a radical derived from 5- to 7-membered aromatic ring (5-7 membered heteroaryl). Heteroaryl includes fused or bridged ring systems. The heteroatom(s) in the heteroaryl radical is optionally oxidized. One or more nitrogen atoms, if present, are optionally quaternized. The heteroaryl is attached to the rest of the molecule through any atom of the ring(s). Examples of such groups include, but not limited to, pyridinyl, imidazolyl, pyrimidinyl, pyrazolyl, triazolyl, pyrazinyl, tetrazolyl, furyl, thienyl, isoxazolyl, thiazolyl, oxazolyl, isothiazolyl, pyrrolyl, quinolinyl, isoquinolinyl, indolyl, benzimidazolyl, benzofuranyl, cinnolinyl, indazolyl, indolizinyl, phthalazinyl, pyridazinyl, triazinyl, isoindolyl, pteridinyl, purinyl, oxadiazolyl, thiadiazolyl, furazanyl, benzofurazanyl, benzothiophenyl, benzothiazolyl, benzoxazolyl, quinazolinyl, quinoxalinyl, naphthyridinyl, furopyridinyl, and the like. In certain embodiments, an heteroaryl is attached to the rest of the molecule via a ring carbon atom. In certain embodiments, an heteroaryl is attached to the rest of the molecule via a nitrogen atom (N-attached) or a carbon atom (C-attached). For instance, a group derived from pyrrole may be pyrrol-1-yl (N-attached) or pyrrol-3-yl (C-attached). Further, a group derived from imidazole may be imidazol-1-yl (N-attached) or imidazol-3-yl (C-attached).

The term “heterocyclyl”, as used herein, means a non-aromatic, monocyclic, bicyclic, tricyclic, or tetracyclic radical having a total of from 4, 5, 6, 7, 8, 9, 10, 11, 12, or 13 atoms in its ring system, and containing from 3 to 12 carbon atoms and from 1 to 4 heteroatoms each independently selected from O, S and N, and with the proviso that the ring of said group does not contain two adjacent 0 atoms or two adjacent S atoms. A heterocyclyl group may include fused, bridged or spirocyclic ring systems. In certain embodiments, a hetercyclyl group comprises 3 to 10 ring atoms (3-10 membered heterocyclyl). In certain embodiments, a hetercyclyl group comprises 3 to 8 ring atoms (3-8 membered heterocyclyl). In certain embodiments, a hetercyclyl group comprises 4 to 10 ring atoms (4-10 membered heterocyclyl). In certain embodiments, a hetercyclyl group comprises 4 to 8 ring atoms (4-8 membered heterocyclyl). A heterocyclyl group may contain an oxo substituent at any available atom that will result in a stable compound. For example, such a group may contain an oxo atom at an available carbon or nitrogen atom. Such a group may contain more than one oxo substituent if chemically feasible. In addition, it is to be understood that when such a heterocyclyl group contains a sulfur atom, said sulfur atom may be oxidized with one or two oxygen atoms to afford either a sulfoxide or sulfone. An example of a 4 membered heterocyclyl group is azetidinyl (derived from azetidine). An example of a 5 membered cycloheteroalkyl group is pyrrolidinyl. An example of a 6 membered cycloheteroalkyl group is piperidinyl. An example of a 9 membered cycloheteroalkyl group is indolinyl. An example of a 10 membered cycloheteroalkyl group is 4H-quinolizinyl. Further examples of such heterocyclyl groups include, but are not limited to, tetrahydrofuranyl, dihydrofuranyl, tetrahydrothienyl, tetrahydropyranyl, dihydropyranyl, tetrahydrothiopyranyl, piperidino, morpholino, thiomorpholino, thioxanyl, piperazinyl, azetidinyl, oxetanyl, thietanyl, homopiperidinyl, oxepanyl, thiepanyl, oxazepinyl, diazepinyl, thiazepinyl, 1,2,3,6-tetrahydropyridinyl, 2-pyrrolinyl, 3-pyrrolinyl, indolinyl, 2H-pyranyl, 4H-pyranyl, dioxanyl, 1,3-dioxolanyl, pyrazolinyl, dithianyl, dithiolanyl, dihydropyranyl, dihydrothienyl, dihydrofuranyl, pyrazolidinyl, imidazolinyl, imidazolidinyl, 3-azabicyclo[3.1.0]hexanyl, 3-azabicyclo[4.1.0]heptanyl, 3H-indolyl, quinolizinyl, 3-oxopiperazinyl, 4-methylpiperazinyl, 4-ethylpiperazinyl, and 1-oxo-2,8, diazaspiro[4.5]dec-8-yl. A heteroaryl group may be attached to the rest of molecular via a carbon atom (C-attached) or a nitrogen atom (N-attached). For instance, a group derived from piperazine may be piperazin-1-yl (N-attached) or piperazin-2-yl (C-attached).

The term “cycloalkyl” or “carbocyclyl” means a saturated, monocyclic, bicyclic, tricyclic, or tetracyclic radical having a total of from 4, 5, 6, 7, 8, 9, 10, 11, 12, or 13 carbon atoms in its ring system. A cycloalkyl may be fused, bridged or spirocyclic. In certain embodiments, a cycloalkyl comprises 3 to 8 carbon ring atoms (3-8 membered carbocyclyl). In certain embodiments, a cycloalkyl comprises 3 to 10 carbon ring atoms (3-10 membered cycloalkyl). Examples of such groups include, but are not limited to, cyclopropyl, cyclobutyl, cyclopentyl, cyclopentenyl, cyclohexyl, cycloheptyl, adamantyl, and the like.

The term “cycloalkylene” is a bidentate radical obtained by removing a hydrogen atom from a cycloalkyl ring as defined above. Examples of such groups include, but are not limited to, cyclopropylene, cyclobutylene, cyclopentylene, cyclopentenylene, cyclohexylene, cycloheptylene, and the like.

As used herein, the term “chain atom” refers to atoms that is located on the main chain of the linker moiety.

The term “spirocyclic” as used herein has its conventional meaning, that is, any ring system containing two or more rings wherein two of the rings have one ring carbon in common. Each ring of the spirocyclic ring system, as herein defined, independently comprises 3 to 20 ring atoms. Preferably, they have 3 to 10 ring atoms. Non-limiting examples of a spirocyclic system include spiro[3.3]heptane, spiro[3.4]octane, and spiro[4.5]decane.

The term cyano” refers to a —C≡N group.

An “aldehyde” group refers to a —C(O)H group.

An “alkoxy” group refers to both an —O-alkyl, as defined herein.

An “alkoxycarbonyl” refers to a —C(O)-alkoxy, as defined herein.

An “alkylaminoalkyl” group refers to an -alkyl-NR-alkyl group, as defined herein.

An “alkylsulfonyl” group refer to a —SO₂alkyl, as defined herein.

An “amino” group refers to an optionally substituted —NH₂.

An “aminoalkyl” group refers to an -alky-amino group, as defined herein.

An “aminocarbonyl” refers to a —C(O)-amino, as defined herein.

An “arylalkyl” group refers to -alkylaryl, where alkyl and aryl are defined herein.

An “aryloxy” group refers to both an —O-aryl and an —O-heteroaryl group, as defined herein.

An “aryloxycarbonyl” refers to —C(O)-aryloxy, as defined herein.

An “arylsulfonyl” group refers to a —SO₂aryl, as defined herein.

A “carbonyl” group refers to a —C(O)— group, as defined herein.

A “carboxylic acid” group refers to a —C(O)OH group.

A “cycloalkoxy” refers to a —O-cycloalkyl group, as defined herein.

A “halo” or “halogen” group refers to fluorine, chlorine, bromine or iodine.

A “haloalkyl” group refers to an alkyl group substituted with one or more halogen atoms.

A “hydroxy” group refers to an —OH group.

A “nitro” group refers to a —NO₂ group.

An “oxo” group refers to the ═O substituent.

A “trihalomethyl” group refers to a methyl substituted with three halogen atoms.

The term “substituted,” means that the specified group or moiety bears one or more substituents independently selected from C₁-C₄ alkyl, aryl, heteroaryl, aryl-C₁-C₄ alkyl-, heteroaryl-C₁-C₄ alkyl-, C₁-C₄ haloalkyl, —OC₁-C₄ alkyl, —OC₁-C₄ alkylphenyl, —C₁-C₄ alkyl-OH, —OC₁-C₄ haloalkyl, halo, —OH, —NH₂, —C₁-C₄ alkyl-NH₂, —N(C₁-C₄ alkyl)(C₁-C₄ alkyl), —NH(C₁-C₄ alkyl), —N(C₁-C₄ alkyl)(C₁-C₄ alkylphenyl), —NH(C₁-C₄ alkylphenyl), cyano, nitro, oxo, —CO₂H, —C(O)OC₁-C₄ alkyl, —CON(C₁-C₄ alkyl)(C₁-C₄ alkyl), —CONH(C₁-C₄ alkyl), —CONH₂, —NHC(O)(C₁-C₄ alkyl), —NHC(O)(phenyl), —N(C₁-C₄ alkyl)C(O)(C₁-C₄ alkyl), —N(C₁-C₄ alkyl)C(O)(phenyl), —C(O)C₁-C₄ alkyl, —C(O)C₁-C₄ alkylphenyl, —C(O)C₁-C₄ haloalkyl, —OC(O)C₁-C₄ alkyl, —SO₂(C₁-C₄ alkyl), —SO₂(phenyl), —SO₂(C₁-C₄ haloalkyl), —SO₂NH₂, —SO₂NH(C₁-C₄ alkyl), —SO₂NH(phenyl), —NHSO₂(C₁-C₄ alkyl), —NHSO₂(phenyl), and —NHSO₂(C₁-C₄ haloalkyl).

The term “null” means the absence of an atom or moiety, and there is a bond between adjacent atoms in the structure.

The term “optionally substituted” means that the specified group may be either unsubstituted or substituted by one or more substituents as defined herein. It is to be understood that in the compounds of the present invention when a group is said to be “unsubstituted,” or is “substituted” with fewer groups than would fill the valencies of all the atoms in the compound, the remaining valencies on such a group are filled by hydrogen. For example, if a C₆ aryl group, also called “phenyl” herein, is substituted with one additional substituent, one of ordinary skill in the art would understand that such a group has 4 open positions left on carbon atoms of the C₆ aryl ring (6 initial positions, minus one at which the remainder of the compound of the present invention is attached to and an additional substituent, remaining 4 positions open). In such cases, the remaining 4 carbon atoms are each bound to one hydrogen atom to fill their valencies. Similarly, if a C₆ aryl group in the present compounds is said to be “disubstituted,” one of ordinary skill in the art would understand it to mean that the C₆ aryl has 3 carbon atoms remaining that are unsubstituted. Those three unsubstituted carbon atoms are each bound to one hydrogen atom to fill their valencies. Unless otherwise specified, an optionally substituted radical may be a radical unsubstituted or substituted with one or more substituents selected from halogen, CN, NO₂, OR^{m}, SR^{m}, NRⁿR^{o}, COR^{m}, CO₂R^{m}, CONRⁿR^{o}, SOR^{m}, SO₂R^{m}, SO₂NRⁿR^{o}, NRⁿCOR^{o}, NR^{m}C(O)NRⁿR^{o}, NRⁿSOR^{o}, NRⁿSO₂R^{o}, C₁-C₈ alkyl, C₁-C₈alkoxyC₁-C₈alkyl, C₁-C₈ haloalkyl, C₁-C₈ hydroxyalkyl, C₁-C₈alkylaminoC₁-C₈alkyl, C₃-C₇ cycloalkyl, 3-7 membered heterocyclyl, C₂-C₈ alkenyl, C₂-C₈ alkynyl, aryl, and heteroaryl, wherein R^{m}, Rⁿ, and R^{o} are independently selected from null, hydrogen, C₁-C₈ alkyl, C₂-C₈ alkenyl, C₂-C₈ alkynyl, C₃-C₇ cycloalkyl, 3-7 membered heterocyclyl, aryl, and heteroaryl, or Rⁿ and R^{o} together with the atom to which they are connected form a 4-8 membered cycloalkyl or heterocyclyl ring.

As used herein, the same symbol in different FORMULA means different definition, for example, the definition of R¹ in FORMULA 1 is as defined with respect to FORMULA 1 and the definition of R¹ is as defined with respect to FORMULA 6.

As used herein, when m (or n or o or p) is defined by a range, for example, “m is 0 to 15” or “m=0-3” mean that m is an integer from 0 to 15 (i.e. m is 0, 1, 2, 3, 4, 5, 6, 7, 8, 9, 10, 11, 12, 13, 14, or 15) or m is an integer from 0 to 3 (i.e. m is 0, 1, 2, or 3) or is any integer in the defined range.

As used herein, (CH₂)_{a-b} (a and b are integer) means a group of (CH₂)_{c}, and c is an integer from a to b (i.e. c is a, a+1, a+2, . . . , b−1, or b). For example, (CH₂)₀₋₃ means a group of null, (CH₂), (CH₂)₂, or (CH₂)₃.

“Pharmaceutically acceptable salt” includes both acid and base addition salts. A pharmaceutically acceptable salt of any one of the bivalent compounds described herein is intended to encompass any and all pharmaceutically suitable salt forms. Preferred pharmaceutically acceptable salts of the compounds described herein are pharmaceutically acceptable acid addition salts and pharmaceutically acceptable base addition salts.

“Pharmaceutically acceptable acid addition salt” refers to those salts which retain the biological effectiveness and properties of the free bases, which are not biologically or otherwise undesirable, and which are formed with inorganic acids such as hydrochloric acid, hydrobromic acid, sulfuric acid, nitric acid, phosphoric acid, hydroiodic acid, hydrofluoric acid, phosphorous acid, and the like. Also included are salts that are formed with organic acids such as aliphatic mono- and dicarboxylic acids, phenyl-substituted alkanoic acids, hydroxy alkanoic acids, alkanedioic acids, aromatic acids, aliphatic and. aromatic sulfonic acids, etc. and include, for example, acetic acid, trifluoroacetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid, and the like. Exemplary salts thus include sulfates, pyrosulfates, bisulfates, sulfites, bisulfates, nitrates, phosphates, monohydrogenphosphates, dihydrogenphosphates, metaphosphates, pyrophosphates, chlorides, bromides, iodides, acetates, trifluoroacetates, propionates, caprylates, isobutyrates, oxalates, malonates, succinate suberates, sebacates, fumarates, maleates, mandelates, benzoates, chlorobenzoates, methylbenzoates, dinitrobenzoates, phthalates, benzenesulfonates, toluenesulfonates, phenylacetates, citrates, lactates, malates, tartrates, methanesulfonates, and the like. Also contemplated are salts of amino acids, such as arginates, gluconates, and galacturonates (see, for example, Berge S. M. et al., “Pharmaceutical Salts,” Journal of Pharmaceutical Science, 66:1-19 (1997), which is hereby incorporated by reference in its entirety). Acid addition salts of basic compounds may be prepared by contacting the free base forms with a sufficient amount of the desired acid to produce the salt according to methods and techniques with which a skilled artisan is familiar.

“Pharmaceutically acceptable base addition salt” refers to those salts that retain the biological effectiveness and properties of the free acids, which are not biologically or otherwise undesirable. These salts are prepared from addition of an inorganic base or an organic base to the free acid. Pharmaceutically acceptable base addition salts may be formed with metals or amines, such as alkali and alkaline earth metals or organic amines. Salts derived from inorganic bases include, but are not limited to, sodium, potassium, lithium, ammonium, calcium, magnesium, iron, zinc, copper, manganese, aluminum salts and the like. Salts derived from organic bases include, but are not limited to, salts of primary, secondary, and tertiary amines, substituted amines including naturally occurring substituted amines, cyclic amines and basic ion exchange resins, for example, isopropylamine, trimethylamine, diethylamine, triethylamine, tripropylamine, ethanolamine, diethanolamine, 2-dimethylaminoethanol, 2-diethylaminoethanol, dicyclohexylamine, lysine, arginine, histidine, caffeine, procaine, N,N-dibenzylethylenediamine, chloroprocaine, hydrabamine, choline, betaine, ethylenediamine, ethylenedianiline, N-methylglucamine, glucosamine, methylglucamine, theobromine, purines, piperazine, piperidine, N-ethylpiperidine, polyamine resins and the like. See Berge et al., supra.

Pharmaceutical Compositions

In some aspects, the compositions and methods described herein include the manufacture and use of pharmaceutical compositions and medicaments that include one or more bivalent compounds as disclosed herein. Also included are the pharmaceutical compositions themselves.

In some aspects, the compositions disclosed herein can include other compounds, drugs, or agents used for the treatment of cancer. For example, in some instances, pharmaceutical compositions disclosed herein can be combined with one or more (e.g., one, two, three, four, five, or less than ten) compounds. Such additional compounds can include, e.g., conventional chemotherapeutic agents or any other cancer treatment known in the art. When co-administered, bivalent compounds disclosed herein can operate in conjunction with conventional chemotherapeutic agents or any other cancer treatment known in the art to produce mechanistically additive or synergistic therapeutic effects.

In some aspects, the pH of the compositions disclosed herein can be adjusted with pharmaceutically acceptable acids, bases, or buffers to enhance the stability of the bivalent compound or its delivery form.

Pharmaceutical compositions typically include a pharmaceutically acceptable excipient, adjuvant, or vehicle. As used herein, the phrase “pharmaceutically acceptable” refers to molecular entities and compositions that are generally believed to be physiologically tolerable and do not typically produce an allergic or similar untoward reaction, such as gastric upset, dizziness and the like, when administered to a human. A pharmaceutically acceptable excipient, adjuvant, or vehicle is a substance that can be administered to a patient, together with a compound of the invention, and which does not compromise the pharmacological activity thereof and is nontoxic when administered in doses sufficient to deliver a therapeutic amount of the compound. Exemplary conventional nontoxic pharmaceutically acceptable excipients, adjuvants, and vehicles include, but not limited to, saline, solvents, dispersion media, coatings, antibacterial and antifungal agents, isotonic and absorption delaying agents, and the like, compatible with pharmaceutical administration.

In particular, pharmaceutically acceptable excipients, adjuvants, and vehicles that can be used in the pharmaceutical compositions of this invention include, but are not limited to, ion exchangers, alumina, aluminum stearate, lecithin, self-emulsifying drug delivery systems (SEDDS) such as d-α-tocopherol polyethylene glycol 1000 succinate, surfactants used in pharmaceutical dosage forms such as Tweens or other similar polymeric delivery matrices, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat. Cyclodextrins such as α-, β-, and γ-cyclodextrin, may also be advantageously used to enhance delivery of compounds of the formulae described herein.

Depending on the dosage form selected to deliver the bivalent compounds disclosed herein, different pharmaceutically acceptable excipients, adjuvants, and vehicles may be used. In the case of tablets for oral use, pharmaceutically acceptable excipients, adjuvants, and vehicles may be used include lactose and corn starch. Lubricating agents, such as magnesium stearate, are also typically added. For oral administration in a capsule form, useful diluents include lactose and dried corn starch. When aqueous suspensions or emulsions are administered orally, the active ingredient may be suspended or dissolved in an oily phase is combined with emulsifying or suspending agents. If desired, certain sweetening, flavoring, or coloring agents can be added.

As used herein, the bivalent compounds disclosed herein are defined to include pharmaceutically acceptable derivatives or prodrugs thereof. A “pharmaceutically acceptable derivative” means any pharmaceutically acceptable salt, solvate, or prodrug, e.g., carbamate, ester, phosphate ester, salt of an ester, or other derivative of a compound or agent disclosed herein, which upon administration to a recipient is capable of providing (directly or indirectly) a compound described herein, or an active metabolite or residue thereof. Particularly favored derivatives and prodrugs are those that increase the bioavailability of the compounds disclosed herein when such compounds are administered to a subject (e.g., by allowing an orally administered compound to be more readily absorbed into the blood) or which enhance delivery of the parent compound to a biological compartment (e.g., the brain or lymphatic system) relative to the parent species. Preferred prodrugs include derivatives where a group that enhances aqueous solubility or active transport through the gut membrane is appended to the structure of formulae described herein. Such derivatives are recognizable to those skilled in the art without undue experimentation. Nevertheless, reference is made to the teaching of Burger's Medicinal Chemistry and Drug Discovery, 5^{th} Edition, Vol. 1: Principles and Practice, which is incorporated herein by reference to the extent of teaching such derivatives.

The bivalent compounds disclosed herein include pure enantiomers, mixtures of enantiomers, pure diastereoisomers, mixtures of diastereoisomers, diastereoisomeric racemates, mixtures of diastereoisomeric racemates and the meso-form and pharmaceutically acceptable salts, solvent complexes, morphological forms, or deuterated derivatives thereof. The single enantiomers or diastereomers, i.e., optically active forms, can be obtained by asymmetric synthesis or by resolution of the racemates. Resolution of the racemates can be accomplished, for example, by conventional methods such as crystallization in the presence of a resolving agent, or chromatography, using, for example a chiral high-pressure liquid chromatography (HPLC) column. In addition, compounds include Z- and E-forms (or cis- and trans-forms) of compounds with carbon-carbon double bonds. Where compounds described herein exist in various tautomeric forms, the term “compound” is intended to include all tautomeric forms of the compound.

The bivalent compounds disclosed herein also include crystalline and amorphous forms of those compounds, including, for example, polymorphs, pseudopolymorphs, solvates (including hydrates), unsolvated polymorphs (including anhydrates), conformational polymorphs, and amorphous forms of the compounds, as well as mixtures thereof. “Crystalline form,” “polymorph,” and “novel form” may be used interchangeably herein, and are meant to include all crystalline and amorphous forms of the compound, including, for example, polymorphs, pseudopolymorphs, solvates (including hydrates), unsolvated polymorphs (including anhydrates), conformational polymorphs, and amorphous forms, as well as mixtures thereof, unless a particular crystalline or amorphous form is referred to. Similarly, “pharmaceutically acceptable salts” of the bivalent compounds also include crystalline and amorphous forms of those compounds, including, for example, polymorphs, pseudopolymorphs, solvates (including hydrates), unsolvated polymorphs (including anhydrates), conformational polymorphs, and amorphous forms of the pharmaceutically acceptable salts, as well as mixtures thereof.

A “solvate” is formed by the interaction of a solvent and a compound. The term “compound” is intended to include solvates of compounds. Similarly, “pharmaceutically acceptable salts” includes solvates of pharmaceutically acceptable salts. Suitable solvates are pharmaceutically acceptable solvates, such as hydrates, including monohydrates and hemi-hydrates.

In some aspects, the pharmaceutical compositions disclosed herein can include an effective amount of one or more bivalent compounds. The terms “effective amount” and “effective to treat,” as used herein, refer to an amount or a concentration of one or more compounds or a pharmaceutical composition described herein utilized for a period of time (including acute or chronic administration and periodic or continuous administration) that is effective within the context of its administration for causing an intended effect or physiological outcome (e.g., treatment or prevention of cell growth, cell proliferation, or cancer). In some aspects, pharmaceutical compositions can further include one or more additional compounds, drugs, or agents used for the treatment of cancer (e.g., conventional chemotherapeutic agents) in amounts effective for causing an intended effect or physiological outcome (e.g., treatment or prevention of cell growth, cell proliferation, or cancer).

In some aspects, the pharmaceutical compositions disclosed herein can be formulated for sale in the United States, import into the United States, or export from the United States.

Administration of Pharmaceutical Compositions

The pharmaceutical compositions disclosed herein can be formulated or adapted for administration to a subject via any route, e.g., any route approved by the Food and Drug Administration (FDA). Exemplary methods are described in the FDA Data Standards Manual (DSM) (available at http://www.fda.gov/Drugs/DevelopmentApprovalProcess/FormsSubmissionRequirements/ElectronicSubmissions/DataStandardsManualmonographs). In particular, the pharmaceutical compositions can be formulated for and administered via oral, parenteral, or transdermal delivery. The term “parenteral” as used herein includes subcutaneous, intracutaneous, intravenous, intramuscular, intraperitoneal, intra-articular, intra-arterial, intrasynovial, intrasternal, intrathecal, intralesional, and intracranial injection or infusion techniques.

For example, the pharmaceutical compositions disclosed herein can be administered, e.g., topically, rectally, nasally (e.g., by inhalation spray or nebulizer), buccally, vaginally, subdermally (e.g., by injection or via an implanted reservoir), or ophthalmically.

For example, pharmaceutical compositions of this invention can be orally administered in any orally acceptable dosage form including, but not limited to, capsules, tablets, emulsions and aqueous suspensions, dispersions and solutions.

For example, the pharmaceutical compositions of this invention can be administered in the form of suppositories for rectal administration. These compositions can be prepared by mixing a compound of this invention with a suitable non-irritating excipient which is solid at room temperature but liquid at the rectal temperature and therefore will melt in the rectum to release the active components. Such materials include, but are not limited to, cocoa butter, beeswax, and polyethylene glycols.

For example, the pharmaceutical compositions of this invention can be administered by nasal aerosol or inhalation. Such compositions are prepared according to techniques well-known in the art of pharmaceutical formulation and can be prepared as solutions in saline, employing benzyl alcohol or other suitable preservatives, absorption promoters to enhance bioavailability, fluorocarbons, or other solubilizing or dispersing agents known in the art.

For example, the pharmaceutical compositions of this invention can be administered by injection (e.g., as a solution or powder). Such compositions can be formulated according to techniques known in the art using suitable dispersing or wetting agents (such as, for example, Tween 80) and suspending agents. The sterile injectable preparation may also be a sterile injectable solution or suspension in a non-toxic parenterally acceptable diluent or solvent, e.g., as a solution in 1,3-butanediol. Among the acceptable vehicles and solvents that may be employed are mannitol, water, Ringer's solution, and isotonic sodium chloride solution. In addition, sterile, fixed oils are conventionally employed as a solvent or suspending medium. For this purpose, any bland fixed oil can be employed, including synthetic mono- or diglycerides. Fatty acids, such as oleic acid and its glyceride derivatives are useful in the preparation of injectables, as are natural pharmaceutically-acceptable oils, e.g., olive oil or castor oil, especially in their polyoxyethylated versions. These oil solutions or suspensions can also contain a long-chain alcohol diluent or dispersant, or carboxymethyl cellulose or similar dispersing agents which are commonly used in the formulation of pharmaceutically acceptable dosage forms such as emulsions and or suspensions. Other commonly used surfactants such as Tweens, Spans, or other similar emulsifying agents or bioavailability enhancers which are commonly used in the manufacture of pharmaceutically acceptable solid, liquid, or other dosage forms can also be used for the purposes of formulation.

In some aspects, an effective dose of a pharmaceutical composition of this invention can include, but is not limited to, e.g., about 0.00001, 0.0001, 0.001, 0.01, 0.02, 0.03, 0.04, 0.05, 0.06, 0.07, 0.08, 0.09, 0.1, 0.15, 0.2, 0.25, 0.3, 0.35, 0.4, 0.45, 0.5, 0.55, 0.6, 0.65, 0.7, 0.75, 0.8, 0.85, 0.9, 0.95, 1, 1.25, 1.5, 1.75, 2, 2.5, 3, 4, 5, 6, 7, 8, 9, 10, 15, 20, 30, 40, 50, 60, 70, 80, 90, 100, 200, 300, 400, 500, 600, 700, 800, 900, 1000, 2500, 5000, or 10000 mg/kg/day, or according to the requirements of the particular pharmaceutical composition.

When the pharmaceutical compositions disclosed herein include a combination of the bivalent compounds described herein and one or more additional compounds (e.g., one or more additional compounds, drugs, or agents used for the treatment of cancer or any other condition or disease, including conditions or diseases known to be associated with or caused by cancer), both the bivalent compounds and the additional compounds may be present at dosage levels of between about 1 to 100%, and more preferably between about 5 to 95% of the dosage normally administered in a monotherapy regimen. The additional agents can be administered separately, as part of a multiple dose regimen, from the compounds of this invention. Alternatively, those agents can be part of a single dosage form, mixed together with the compounds of this invention in a single composition.

In some aspects, the pharmaceutical compositions disclosed herein can be included in a container, pack, or dispenser together with instructions for administration.

Methods of Treatment

The methods disclosed herein contemplate administration of an effective amount of a compound or composition to achieve the desired or stated effect. Typically, the compounds or compositions of the invention will be administered from about 1 to about 6 times per day or, alternately or in addition, as a continuous infusion. Such administration can be used as a chronic or acute therapy. The amount of active ingredient that can be combined with the carrier materials to produce a single dosage form will vary depending upon the host treated and the particular mode of administration. A typical preparation will contain from about 5% to about 95% active compound (w/w). Alternatively, such preparations can contain from about 20% to about 80% active compound.

In some aspects, provided herein are a bivalent compound described herein for preventing or treating a disease or condition.

In some aspects, provided herein are a bivalent compound described herein for treating or preventing one or more diseases or conditions disclosed herein in a subject in need thereof. In certain embodiments, the disease or condition is a CBP/P300-mediated disease or condition. In certain embodiments, the disease or condition is resulted from CBP/P300 expression, mutation, deletion, or fusion. In certain embodiments, the disease or condition is a cancer. In certain embodiments, the disease or condition comprises acoustic neuroma, acute leukemia, acute lymphocytic leukemia, acute myelocytic leukemia, acute T-cell leukemia, basal cell carcinoma, bile duct carcinoma, bladder cancer, brain choriocarcinoma, chronic leukemia, chronic lymphocytic leukemia, chronic myelocytic leukemia, chronic myelogenous leukemia, colon cancer, colorectal cancer, craniopharyngioma, cystadenocarcinoma, diffuse large B-cell lymphoma, dysproliferative changes, embryonal carcinoma, endometrial cancer, endotheliosarcoma, ependymoma, epithelial carcinoma, erythroleukemia, esophageal cancer, estrogen-receptor positive breast cancer, essential thrombocythemia, Ewing's tumor, fibrosarcoma, follicular lymphoma, germ cell testicular cancer, glioma, glioblastoma, gliosarcoma, heavy chain disease, head and neck cancer, hemangioblastoma, hepatoma, hepatocellular cancer, hormone insensitive prostate cancer, leiomyosarcoma, leukemia, liposarcoma, lung cancer, lymphagioendotheliosarcoma, lymphangiosarcoma, lymphoblastic leukemia, lymphoma, lymphoid malignancies of T-cell or B-cell origin, medullary carcinoma, medulloblastoma, melanoma, meningioma, mesothelioma, multiple myeloma, myelogenous leukemia, myeloma, myxosarcoma, neuroblastoma, NUT midline carcinoma (NMC), non-small cell lung cancer, oligodendroglioma, oral cancer, osteogenic sarcoma, ovarian cancer, pancreatic cancer, papillary adenocarcinomas, papillary carcinoma, pinealoma, polycythemia vera, prostate cancer, rectal cancer, renal cell carcinoma, retinoblastoma, rhabdomyosarcoma, sarcoma, sebaceous gland carcinoma, seminoma, skin cancer, small cell lung carcinoma, solid tumors (carcinomas and sarcomas), small cell lung cancer, stomach cancer, squamous cell carcinoma, synovioma, sweat gland carcinoma, thyroid cancer, Waldenstrom's macroglobulinemia, testicular tumors, uterine cancer, and Wilms' tumor. In certain embodiments, the disease or condition is a relapsed cancer. In certain embodiments, the disease or condition is an inflammatory disorder or the autoimmune disease. In certain embodiments, the disease or condition comprises Addison's disease, acute gout, ankylosing spondylitis, asthma, atherosclerosis, Behcet's disease, bullous skin diseases, chronic obstructive pulmonary disease, Crohn's disease, dermatitis, eczema, giant cell arteritis, fibrosis, glomerulonephritis, hepatic vascular occlusion, hepatitis, hypophysitis, immunodeficiency syndrome, inflammatory bowel disease, Kawasaki disease, lupus nephritis, multiple sclerosis, myocarditis, myositis, nephritis, organ transplant rejection, osteoarthritis, pancreatitis, pericarditis, Polyarteritis nodosa, pneumonitis, primary biliary cirrhosis, psoriasis, psoriatic arthritis, rheumatoid arthritis, scleritis, sclerosing cholangitis, sepsis, systemic lupus erythematosus, Takayasu's Arteritis, toxic shock, thyroiditis, type I diabetes, ulcerative colitis, uveitis, vitiligo, vasculitis, and Wegener's granulomatosis. In certain embodiments, the disease or condition is refractory to one or more previous treatments.

In some aspects, provided herein are use of a bivalent compound in manufacture of a medicament for preventing or treating one or more diseases or conditions disclosed herein.

In some aspects, the methods disclosed include the administration of a therapeutically effective amount of one or more of the compounds or compositions described herein to a subject (e.g., a mammalian subject, e.g., a human subject) who is in need of, or who has been determined to be in need of, such treatment. In some aspects, the methods disclosed include selecting a subject and administering to the subject an effective amount of one or more of the compounds or compositions described herein, and optionally repeating administration as required for the prevention or treatment of cancer.

In some aspects, subject selection can include obtaining a sample from a subject (e.g., a candidate subject) and testing the sample for an indication that the subject is suitable for selection. In some aspects, the subject can be confirmed or identified, e.g. by a health care professional, as having had, having an elevated risk to have, or having a condition or disease. In some aspects, suitable subjects include, for example, subjects who have or had a condition or disease but that resolved the disease or an aspect thereof, present reduced symptoms of disease (e.g., relative to other subjects (e.g., the majority of subjects) with the same condition or disease), or that survive for extended periods of time with the condition or disease (e.g., relative to other subjects (e.g., the majority of subjects) with the same condition or disease), e.g., in an asymptomatic state (e.g., relative to other subjects (e.g., the majority of subjects) with the same condition or disease). In some aspects, exhibition of a positive immune response towards a condition or disease can be made from patient records, family history, or detecting an indication of a positive immune response. In some aspects, multiple parties can be included in subject selection. For example, a first party can obtain a sample from a candidate subject and a second party can test the sample. In some aspects, subjects can be selected or referred by a medical practitioner (e.g., a general practitioner). In some aspects, subject selection can include obtaining a sample from a selected subject and storing the sample or using the in the methods disclosed herein. Samples can include, e.g., cells or populations of cells.

In some aspects, methods of treatment can include a single administration, multiple administrations, and repeating administration of one or more compounds disclosed herein as required for the prevention or treatment of the disease or condition disclosed herein (e.g., an CBP/P300-mediated disease). In some aspects, methods of treatment can include assessing a level of disease in the subject prior to treatment, during treatment, or after treatment. In some aspects, treatment can continue until a decrease in the level of disease in the subject is detected.

The term “subject,” as used herein, refers to any animal. In some instances, the subject is a mammal. In some instances, the term “subject,” as used herein, refers to a human (e.g., a man, a woman, or a child).

The terms “administer,” “administering,” or “administration,” as used herein, refer to implanting, ingesting, injecting, inhaling, or otherwise absorbing a compound or composition, regardless of form. For example, the methods disclosed herein include administration of an effective amount of a compound or composition to achieve the desired or stated effect.

The terms “treat”, “treating,” or “treatment,” as used herein, refer to partially or completely alleviating, inhibiting, ameliorating, or relieving the disease or condition from which the subject is suffering. This means any manner in which one or more of the symptoms of a disease or disorder (e.g., cancer) are ameliorated or otherwise beneficially altered. As used herein, amelioration of the symptoms of a particular disorder (e.g., cancer) refers to any lessening, whether permanent or temporary, lasting or transient that can be attributed to or associated with treatment by the bivalent compounds, compositions and methods of the present invention. In some embodiments, treatment can promote or result in, for example, a decrease in the number of tumor cells (e.g., in a subject) relative to the number of tumor cells prior to treatment; a decrease in the viability (e.g., the average/mean viability) of tumor cells (e.g., in a subject) relative to the viability of tumor cells prior to treatment; a decrease in the rate of growth of tumor cells; a decrease in the rate of local or distant tumor metastasis; or reductions in one or more symptoms associated with one or more tumors in a subject relative to the subject's symptoms prior to treatment.

The terms “prevent,” “preventing,” and “prevention,” as used herein, shall refer to a decrease in the occurrence of a disease or decrease in the risk of acquiring a disease or its associated symptoms in a subject. The prevention may be complete, e.g., the total absence of disease or pathological cells in a subject. The prevention may also be partial, such that the occurrence of the disease or pathological cells in a subject is less than, occurs later than, or develops more slowly than that which would have occurred without the present invention. In certain embodiments, the subject has an elevated risk of developing one or more CBP/P300-mediated diseases. Exemplary CBP/P300-mediated diseases that can be treated with bivalent compounds include, for example, acoustic neuroma, acute leukemia, acute lymphocytic leukemia, acute myelocytic leukemia, acute T-cell leukemia, basal cell carcinoma, bile duct carcinoma, bladder cancer, brain choriocarcinoma, chronic leukemia, chronic lymphocytic leukemia, chronic myelocytic leukemia, chronic myelogenous leukemia, colon cancer, colorectal cancer, craniopharyngioma, cystadenocarcinoma, diffuse large B-cell lymphoma, dysproliferative changes, embryonal carcinoma, endometrial cancer, endotheliosarcoma, ependymoma, epithelial carcinoma, erythroleukemia, esophageal cancer, estrogen-receptor positive breast cancer, essential thrombocythemia, Ewing's tumor, fibrosarcoma, follicular lymphoma, germ cell testicular cancer, glioma, glioblastoma, gliosarcoma, heavy chain disease, head and neck cancer, hemangioblastoma, hepatoma, hepatocellular cancer, hormone insensitive prostate cancer, leiomyosarcoma, leukemia, liposarcoma, lung cancer, lymphagioendotheliosarcoma, lymphangiosarcoma, lymphoblastic leukemia, lymphoma, lymphoid malignancies of T-cell or B-cell origin, medullary carcinoma, medulloblastoma, melanoma, meningioma, mesothelioma, multiple myeloma, myelogenous leukemia, myeloma, myxosarcoma, neuroblastoma, NUT midline carcinoma (NMC), non-small cell lung cancer, oligodendroglioma, oral cancer, osteogenic sarcoma, ovarian cancer, pancreatic cancer, papillary adenocarcinomas, papillary carcinoma, pinealoma, polycythemia vera, prostate cancer, rectal cancer, renal cell carcinoma, retinoblastoma, rhabdomyosarcoma, sarcoma, sebaceous gland carcinoma, seminoma, skin cancer, small cell lung carcinoma, solid tumors (carcinomas and sarcomas), small cell lung cancer, stomach cancer, squamous cell carcinoma, synovioma, sweat gland carcinoma, thyroid cancer, Waldenstrom's macroglobulinemia, testicular tumors, uterine cancer, Wilms' tumor, Addison's disease, acute gout, ankylosing spondylitis, asthma, atherosclerosis, Behcet's disease, bullous skin diseases, chronic obstructive pulmonary disease, Crohn's disease, dermatitis, eczema, giant cell arteritis, fibrosis, glomerulonephritis, hepatic vascular occlusion, hepatitis, hypophysitis, immunodeficiency syndrome, inflammatory bowel disease, Kawasaki disease, lupus nephritis, multiple sclerosis, myocarditis, myositis, nephritis, organ transplant rejection, osteoarthritis, pancreatitis, pericarditis, Polyarteritis nodosa, pneumonitis, primary biliary cirrhosis, psoriasis, psoriatic arthritis, rheumatoid arthritis, scleritis, sclerosing cholangitis, sepsis, systemic lupus erythematosus, Takayasu's Arteritis, toxic shock, thyroiditis, type I diabetes, ulcerative colitis, uveitis, vitiligo, vasculitis, and Wegener's granulomatosis.

Specific dosage and treatment regimens for any particular patient will depend upon a variety of factors, including the activity of the specific compound employed, the age, body weight, general health status, sex, diet, time of administration, rate of excretion, drug combination, the severity and course of the disease, condition or symptoms, the patient's disposition to the disease, condition or symptoms, and the judgment of the treating physician.

An effective amount can be administered in one or more administrations, applications or dosages. A therapeutically effective amount of a therapeutic compound (i.e., an effective dosage) depends on the therapeutic compounds selected. Moreover, treatment of a subject with a therapeutically effective amount of the compounds or compositions described herein can include a single treatment or a series of treatments. For example, effective amounts can be administered at least once. The compositions can be administered from one or more times per day to one or more times per week; including once every other day. The skilled artisan will appreciate that certain factors can influence the dosage and timing required to effectively treat a subject, including but not limited to the severity of the disease or disorder, previous treatments, the general health or age of the subject, and other diseases present.

Following administration, the subject can be evaluated to detect, assess, or determine their level of disease. In some instances, treatment can continue until a change (e.g., reduction) in the level of disease in the subject is detected. Upon improvement of a patient's condition (e.g., a change (e.g., decrease) in the level of disease in the subject), a maintenance dose of a compound, or composition disclosed herein can be administered, if necessary. Subsequently, the dosage or frequency of administration, or both, can be reduced, e.g., as a function of the symptoms, to a level at which the improved condition is retained. Patients may, however, require intermittent treatment on a long-term basis upon any recurrence of disease symptoms.

The present disclosure is also described and demonstrated by way of the following examples. However, the use of these and other examples anywhere in the specification is illustrative only and in no way limits the scope and meaning of the invention or of any exemplified term. Likewise, the invention is not limited to any particular preferred embodiment or aspect described herein. Indeed, many modifications and variations may be apparent to those skilled in the art upon reading this specification, and such variations can be made without departing from the invention in spirit or in scope. The invention is therefore to be limited only by the terms of the appended claims along with the full scope of equivalents to which those claims are entitled.

### EXAMPLES

Procedures for the Synthesis of Linker-Attached Degradation Tags for Bivalent Compounds.

### Example 1: 4-((2-Aminoethyl)amino)-2-(2,6-dioxopiperidin-3-yl)isoindoline-1,3-dione (Linker 1)

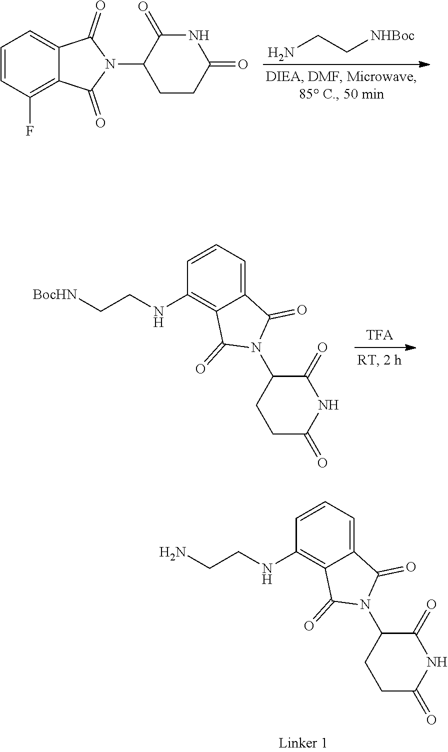

A solution of 2-(2,6-dioxopiperidin-3-yl)-4-fluoroisoindoline-1,3-dione (1.66 g, 6.0 mmol), tert-butyl (2-aminoethyl)carbamate (1.25 g, 6.6 mmol) and N,N-diisopropylethylamine (2.32 g, 18 mmmol) in DMF (12 mL) was heated to 85° C. in a microwave reactor for 50 min. Three batches were combined and diluted with EtOAc (200 mL). The reaction was washed with water and brine. The separated organic layer was dried over anhydrous sodium sulfate and concentrated under reduced pressure. The resulting residue was purified by silica gel chromatography (eluted with hexanes/EtOAc=1:1) to give tert-butyl (2-((2-(2,6-dioxopiperidin-3-yl)-1,3-dioxoisoindolin-4-yl)amino)ethyl)carbamate (1.3 g, yield: 16%) as a yellow solid. MS (ESI) m/z=317.1 [M−100+H]+. A solution of tert-butyl (2-((2-(2,6-dioxopiperidin-3-yl)-1,3-dioxoisoindolin-4-yl) amino)ethyl)carbamate (2.0 g, 4.5 mmol) in DCM (10 mL) and TFA (5 mL) was stirred at rt for 2 h. The reaction was concentrated and triturated with EtOAc. The solid precipitate was filtered. And the solid was washed with MTBE, and dried to give 4-((2-aminoethyl)amino)-2-(2,6-dioxopiperidin-3-yl)isoindoline-1,3-dione as a yellow solid (Linker 1) (1.3 g, yield: 98%). ¹H NMR (400 MHz, DMSO-d₆) δ 11.14 (s, 1H), 7.85 (s, 3H), 7.45 (t, J=7.2 Hz, 1H), 7.19 (d, J=7.2 Hz, 1H), 7.10 (d, J=7.2 Hz, 1H), 6.84 (t, J=6.4 Hz, 1H), 5.07 (dd, J=5.2, 12.8 Hz, 1H), 3.58 (q, J=6.4 Hz, 2H), 3.00 (s, 2H), 2.94-2.85 (m, 1H), 2.62-2.50 (m, 2H), 2.05-2.00 (m, 1H). MS (ESI) m/z=317.1 [M+H]⁺.

### Example 2: 4-((3-Aminopropyl)amino)-2-(2,6-dioxopiperidin-3-yl)isoindoline-1,3-dione (Linker 2)

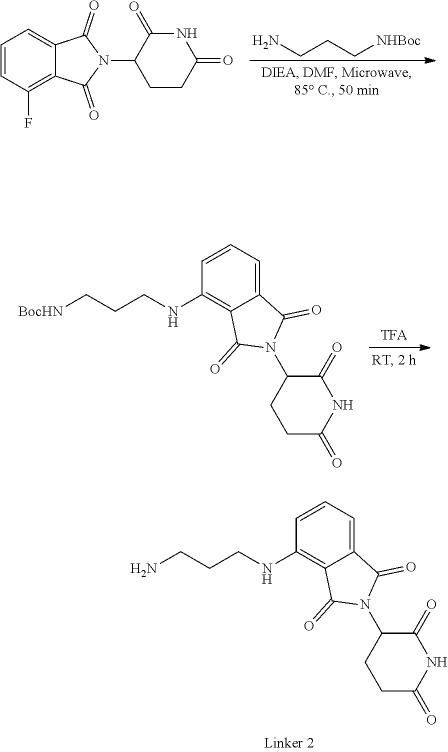

Linker 2 was synthesized following the same procedures as Linker 1 as described in Example 1. (1.2 g, yield: 11% over 2 steps). ¹H NMR (400 MHz, DMSO-d6) 11.11 (s, 1H), 7.74 (s, 3H), 7.62-7.58 (m, 1H), 7.15 (d, J=8.4 Hz, 1H), 7.05 (d, J=7.2 Hz, 1H), 6.78-6.75 (m, 1H), 5.08-5.04 (m, 1H), 3.43-3.36 (m, 2H), 2.90-2.86 (m, 3H), 2.62-2.51 (m, 2H), 2.08-2.01 (m, 1H), 1.86-1.80 (m, 2H). MS (ESI) m/z=331.1 [M+H]⁺

### Example 3: 4-((4-Aminobutyl)amino)-2-(2,6-dioxopiperidin-3-yl)isoindoline-1,3-dione (Linker 3)

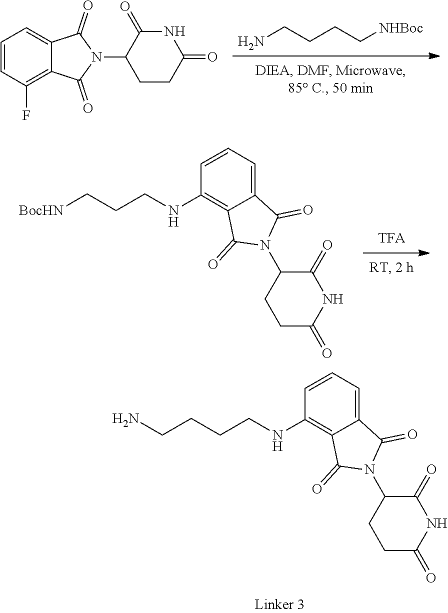

Linker 3 was synthesized following the same procedures as Linker 1 as described in Example 1. (1.4 g, yield: 15% over 2 steps). 1H NMR (400 MHz, DMSO-d6) 11.11 (s, 1H), 7.84 (s, 3H), 7.62-7.57 (m, 1H), 7.13 (d, J=8.4 Hz, 1H), 7.04 (d, J=6.8 Hz, 1H), 6.62 (s, 1H), 5.08-5.04 (m, 1H), 3.34 (s, 2H), 2.90-2.83 (m, 3H), 2.62-2.51 (m, 2H), 2.06-2.01 (m, 1H), 1.65-1.60 (m, 4H). MS (ESI) m/z=345.1 [M+H]+

### Example 4: 4-((5-Aminopentyl)amino)-2-(2,6-dioxopiperidin-3-yl)isoindoline-1,3-dione (Linker 4)

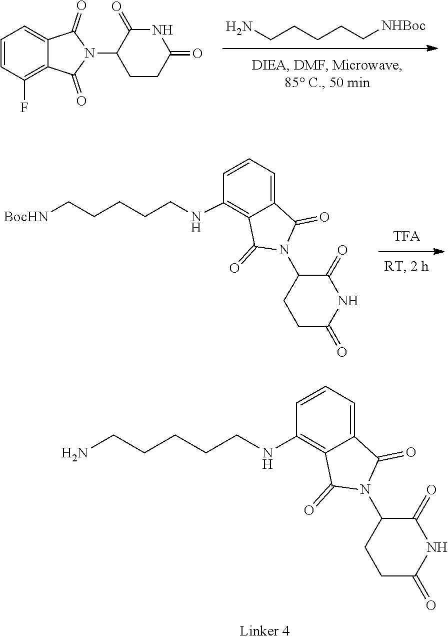

Linker 4 was synthesized following the same procedures as Linker 1 as described in Example 1. (2.3 g, yield: 26% over 2 steps). ¹H NMR (400 MHz, DMSO-d₆) δ11.14 (s, 1H), 7.72 (s, 3H), 7.61-7.57 (m, 1H), 7.10 (d, J=8.4 Hz, 1H), 7.03 (d, J=7.2 Hz, 1H), 6.56-6.53 (m, 1H), 5.07-5.03 (m, 1H), 3.32-3.28 (m, 2H), 2.90-2.78 (m, 3H), 2.62-2.51 (m, 2H), 2.05-1.90 (m, 1H), 1.62-1.54 (m, 4H), 1.41-1.37 (m, 2H). MS (ESI) m/z=359.1 [M+H]⁺

### Example 5: 4-((6-Aminohexyl)amino)-2-(2,6-dioxopiperidin-3-yl)isoindoline-1,3-dione (Linker 5)

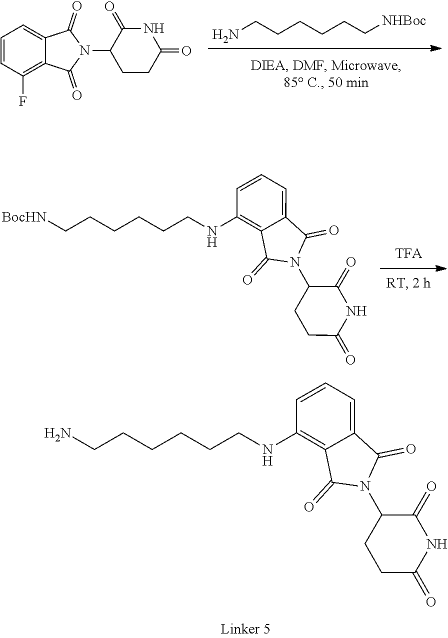

Linker 5 was synthesized following the same procedures as Linker 1 as described in Example 1. (1.8 g, yield: 20% over 2 steps). ¹H NMR (400 MHz, DMSO-d6) δ11.10 (s, 1H), 7.76 (s, 3H), 7.58 (t, J=7.2 Hz, 1H), 7.10 (d, J=8.4 Hz, 1H), 7.03 (d, J=7.2 Hz, 1H), 6.54 (t, J=6.0 Hz, 1H), 5.07-5.03 (m, 1H), 3.37-3.27 (m, 2H), 2.88-2.78 (m, 3H), 2.61-2.50 (m, 2H), 2.04-2.01 (m, 1H), 1.57-1.52 (m, 4H), 1.40-1.30 (m, 4H). MS (ESI) m/z=373.1 [M+H]⁺

### Example 6: 4-((7-Aminoheptyl)amino)-2-(2,6-dioxopiperidin-3-yl)isoindoline-1,3-dione (Linker 6)

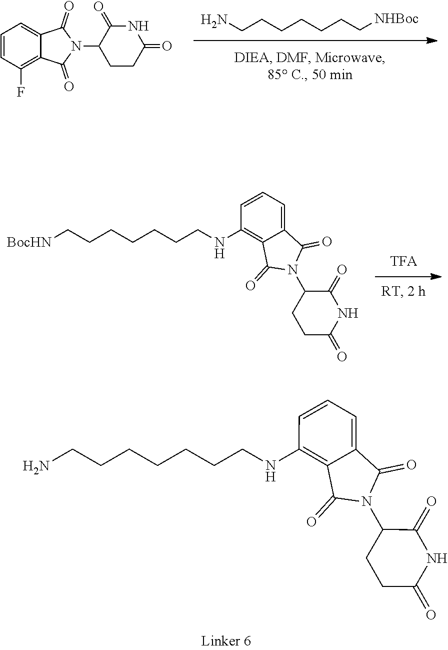 [SMILES: O=C1CCC(N2C(=O)c3cccc(F)c3C2=O)C(=O)N1 ; CC(C)(C)OC(=O)NCCCCCCCN ; CC(C)(C)OC(=O)NCCCCCCCNc1cccc2c1C(=O)N(C1CCC(=O)NC1=O)C2=O ; NCCCCCCCNc1cccc2c1C(=O)N(C1CCC(=O)NC1=O)C2=O]

Linker 6 was synthesized following the same procedures as Linker 1 as described in Example 1. (2.0 g, yield: 25% over 2 steps). ¹H NMR (400 MHz, DMSO-d6) δ11.05 (br, 1H), 7.94-7.56 (m, 4H), 7.10-7.02 (m, 2H), 6.52 (t, J=6.0 Hz, 1H), 5.07-5.02 (m, 1H), 3.32-3.27 (m, 2H), 2.88-2.77 (m, 1H), 2.75-2.61 (m, 2H), 2.60-2.50 (m, 2H), 2.04-2.02 (m, 1H), 1.59-1.50 (m, 4H), 1.35-1.30 (m, 6H). MS (ESI) m/z=387.2 [M+H]⁺

### Example 7: 4-((8-Aminooctyl)amino)-2-(2,6-dioxopiperidin-3-yl)isoindoline-1,3-dione (Linker 7)

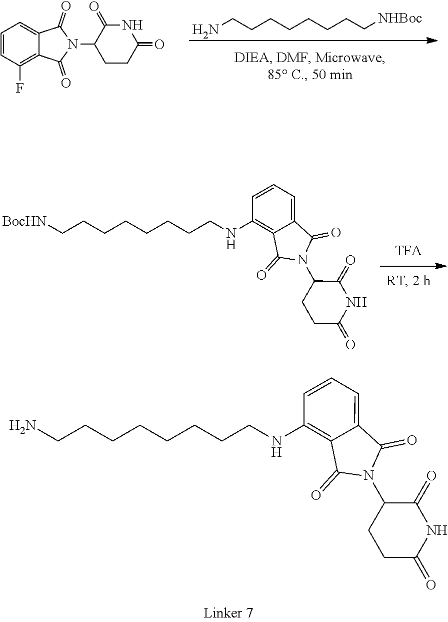 [SMILES: O=C1CCC(N2C(=O)c3cccc(F)c3C2=O)C(=O)N1 ; CC(C)(C)OC(=O)NCCCCCCCCN ; CC(C)(C)OC(=O)NCCCCCCCCNc1cccc2c1C(=O)N(C1CCC(=O)NC1=O)C2=O ; NCCCCCCCCNc1cccc2c1C(=O)N(C1CCC(=O)NC1=O)C2=O]

Linker 7 was synthesized following the same procedures as Linker 1 as described in Example 1. (1.1 g, yield: 18% over 2 steps). ¹H NMR (400 MHz, DMSO-d₆) δ11.10 (s, 1H), 7.69-7.56 (m, 4H), 7.09 (d, J=8.4 Hz, 1H), 7.03 (d, J=6.8 Hz, 1H), 6.52 (t, J=6.0 Hz, 1H), 5.07-5.03 (m, 1H), 3.34-3.26 (m, 2H), 2.89-2.85 (m, 1H), 2.76 (s, 2H), 2.61-2.56 (m, 2H), 2.04-2.00 (m, 1H), 1.59-1.49 (m, 4H), 1.35-1.27 (m, 8H). MS (ESI) m/z=401.2 [M+H]⁺

### Example 8: 4-((2-(2-Aminoethoxy)ethyl)amino)-2-(2,6-dioxopiperidin-3-yl)isoindoline-1,3-dione (Linker 8)

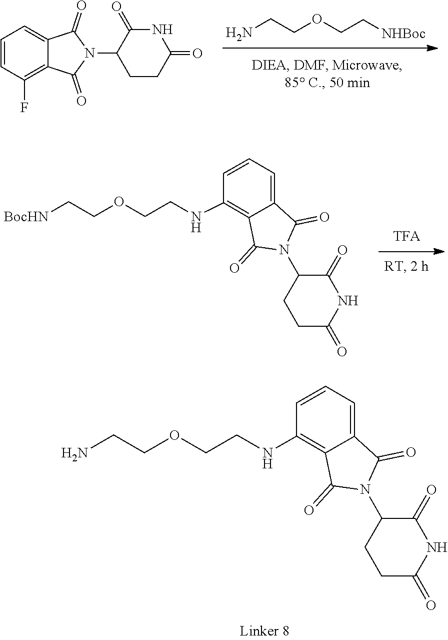

Linker 8 was synthesized following the same procedures as Linker 1 as described in Example 1. (2.0 g, yield: 23% over 2 steps). ¹H NMR (400 MHz, DMSO-d₆) δ 10.10 (s, 1H), 7.88 (s, 3H), 7.60 (t, J=8.0 Hz, 1H), 7.17 (d, J=8.4 Hz, 1H), 7.06 (d, J=6.8 Hz, 1H), 6.40 (d, J=5.6 Hz, 1H), 5.05 (dd, J=5.2, 12.8 Hz, 1H), 3.67-3.62 (m, 4H), 3.54-3.50 (m, 2H), 3.00 (s, 2H), 2.90-2.85 (m, 1H), 2.62-2.50 (m, 2H), 2.03 (t, J=7.6 Hz, 1H). MS (ESI) m/z=361.1 [M+H]⁺

### Example 9: 4-((2-(2-(2-Aminoethoxy)ethoxy)ethyl)amino)-2-(2,6-dioxopiperidin-3-yl)isoindoline-1,3-dione (Linker 9)

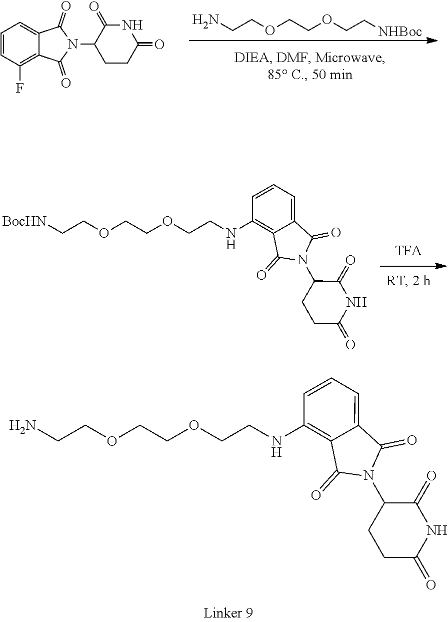 [SMILES: O=C1CCC(N2C(=O)c3cccc(F)c3C2=O)C(=O)N1 ; CC(C)(C)OC(=O)NCCOCCOCCN ; CC(C)(C)OC(=O)NCCOCCOCCNc1cccc2c1C(=O)N(C1CCC(=O)NC1=O)C2=O ; NCCOCCOCCNc1cccc2c1C(=O)N(C1CCC(=O)NC1=O)C2=O]

Linker 9 was synthesized following the same procedures as Linker 1 as described in Example 1. (1.1 g, yield: 17% over 2 steps). ¹H NMR (400 MHz, DMSO-d6) δ 11.11 (s, 1H), 7.84 (s, 3H), 7.62-7.58 (m, 1H), 7.15 (d, J=8.8 Hz, 1H), 7.05 (d, J=6.8 Hz, 1H), 6.62-6.59 (m, 1H), 5.08-5.04 (m, 1H), 3.65-3.59 (m, 8H), 3.50-3.46 (m, 2H), 2.97-2.86 (m, 3H), 2.62-2.51 (m, 2H), 2.05-1.99 (m, 1H). MS (ESI) m/z=405.2 [M+H]⁺

### Example 10: 4-((2-(2-(2-(2-Aminoethoxy)ethoxy)ethoxy)ethyl)amino)-2-(2,6-dioxopiperidin-3-yl)isoindoline-1,3-dione (Linker 10)

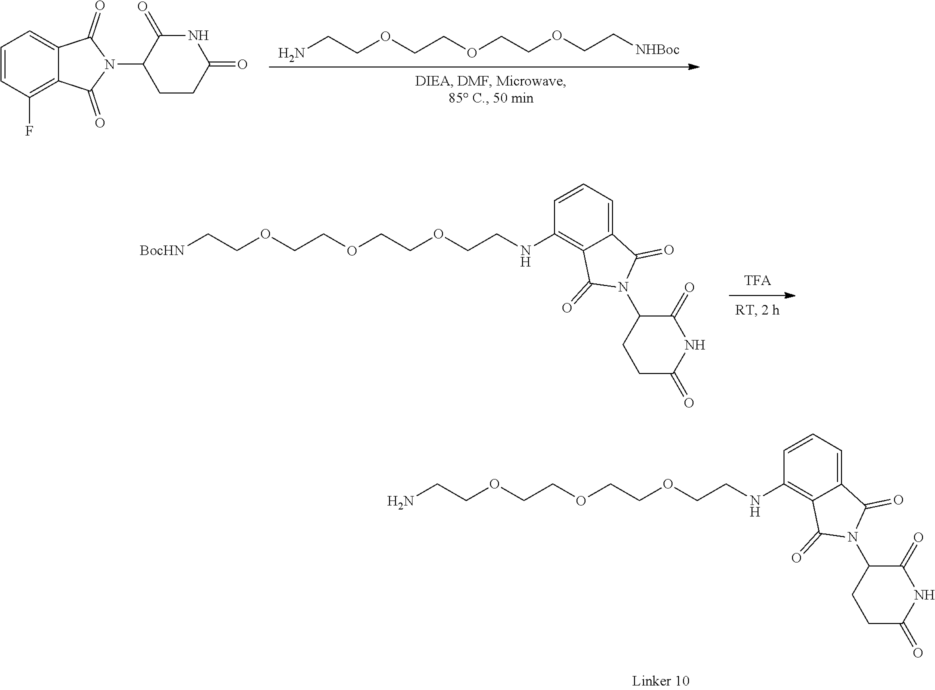 [SMILES: O=C1CCC(N2C(=O)c3cccc(F)c3C2=O)C(=O)N1 ; CC(C)(C)OC(=O)NCCOCCOCCOCCN ; CC(C)(C)OC(=O)NCCOCCOCCOCCNc1cccc2c1C(=O)N(C1CCC(=O)NC1=O)C2=O ; NCCOCCOCCOCCNc1cccc2c1C(=O)N(C1CCC(=O)NC1=O)C2=O]

Linker 10 was synthesized following the same procedures as Linker 1 as described in Example 1. (1.3 g, yield: 17% over 2 steps). ¹H NMR (400 MHz, DMSO-d6) δ 11.11 (s, 1H), 7.83 (s, 3H), 7.61-7.57 (m, 1H), 7.15 (d, J=8.8 Hz, 1H), 7.05 (d, J=6.8 Hz, 1H), 6.62-6.59 (m, 1H), 5.08-5.04 (m, 1H), 3.64-3.45 (m, 14H), 2.97-2.86 (m, 3H), 2.62-2.51 (m, 2H), 2.08-2.01 (m, 1H). MS (ESI) m/z=449.2 [M+H]⁺

### Example 11: 4-((14-Amino-3,6,9,12-tetraoxatetradecyl)amino)-2-(2,6-dioxopiperidin-3-yl)isoindoline-1,3-dione (Linker 11)

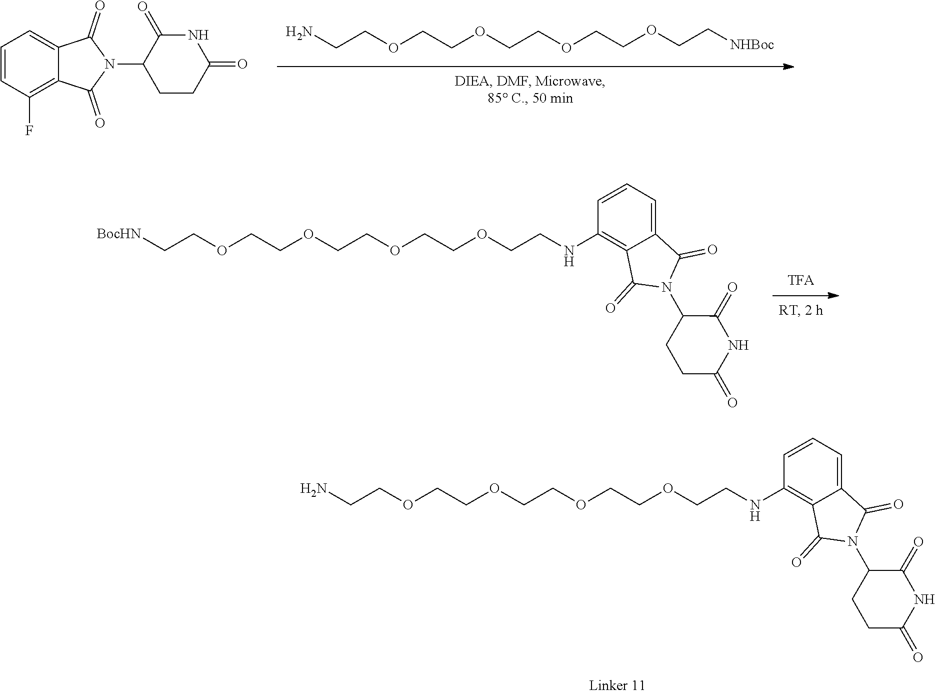 [SMILES: O=C1CCC(N2C(=O)c3cccc(F)c3C2=O)C(=O)N1 ; CC(C)(C)OC(=O)NCCOCCOCCOCCOCCN ; CC(C)(C)OC(=O)NCCOCCOCCOCCOCCNc1cccc2c1C(=O)N(C1CCC(=O)NC1=O)C2=O ; NCCOCCOCCOCCOCCNc1cccc2c1C(=O)N(C1CCC(=O)NC1=O)C2=O]

Linker 11 was synthesized following the same procedures as Linker 1 as described in Example 1. (1.2 g, yield: 16% over 2 steps). ¹H NMR (400 MHz, DMSO-d6) δ 11.11 (s, 1H), 7.84 (s, 3H), 7.61-7.57 (m, 1H), 7.15 (d, J=8.8 Hz, 1H), 7.05 (d, J=6.8 Hz, 1H), 6.61 (s, 1H), 5.08-5.04 (m, 1H), 3.64-3.47 (m, 18H), 2.99-2.86 (m, 3H), 2.62-2.51 (m, 2H), 2.08-2.01 (m, 1H). MS (ESI) m/z=493.2 [M+H]⁺

### Example 12: 4-((17-Amino-3,6,9,12,15-pentaoxaheptadecyl)amino)-2-(2,6-dioxopiperidin-3-yl)isoindoline-1,3-dione (Linker 12)

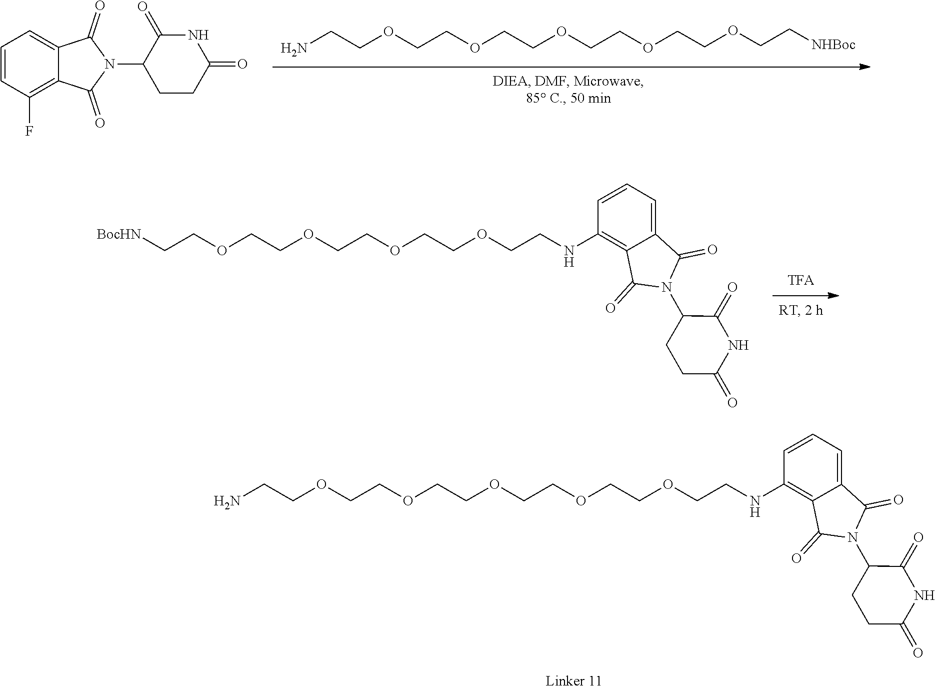 [SMILES: O=C1CCC(N2C(=O)c3cccc(F)c3C2=O)C(=O)N1 ; CC(C)(C)OC(=O)NCCOCCOCCOCCOCCOCCN ; CC(C)(C)OC(=O)NCCOCCOCCOCCOCCNc1cccc2c1C(=O)N(C1CCC(=O)NC1=O)C2=O ; NCCOCCOCCOCCOCCOCCNc1cccc2c1C(=O)N(C1CCC(=O)NC1=O)C2=O]

Linker 12 was synthesized following the same procedures as Linker 1 as described in Example 1. (1.2 g, yield: 15% over 2 steps). ¹H NMR (400 MHz, DMSO-d6) δ 11.11 (s, 1H), 7.82 (s, 3H), 7.61-7.57 (m, 1H), 7.15 (d, J=8.4 Hz, 1H), 7.05 (d, J=7.2 Hz, 1H), 6.61-6.59 (m, 1H), 5.08-5.03 (m, 1H), 3.64-3.47 (m, 22H), 3.00-2.86 (m, 3H), 2.62-2.51 (m, 2H), 2.05-2.02 (m, 1H). MS (ESI) m/z=537.2 [M+H]⁺

### Example 13: (2-(2,6-Dioxopiperidin-3-yl)-1,3-dioxoisoindolin-4-yl)glycine (Linker 13)

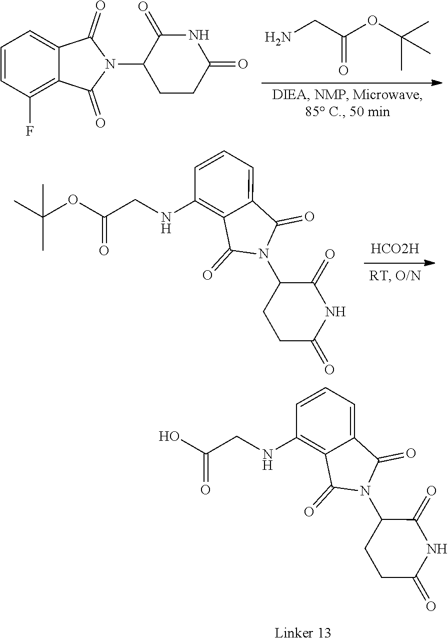

Linker 13 was synthesized following the same procedures as Linker 1 as described in Example 1. (840 mg, yield: 16% over 2 steps). ¹H NMR (400 MHz, DMSO-d6) δ 11.07 (s, 1H), 7.52 (t, J=7.6 Hz, 1H), 6.99-6.88 (m, 3H), 5.04 (dd, J=5.2, 12.8 Hz, 1H), 3.73 (s, 2H), 2.93-2.83 (m, 1H), 2.61-2.50 (m, 2H), 2.02 (t, J=5.6 Hz, 1H). MS (ESI) m/z=330.1 [M−H]⁻

### Example 14: 3-((2-(2,6-Dioxopiperidin-3-yl)-1,3-dioxoisoindolin-4-yl)amino)propanoic acid (Linker 14)

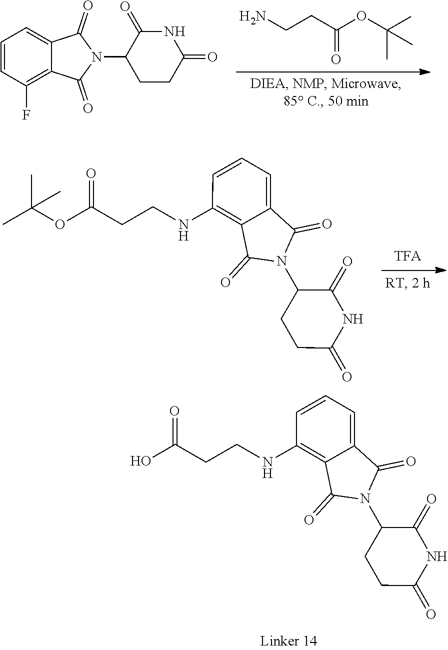

Linker 14 was synthesized following the same procedures as Linker 1 as described in Example 1. (1.42 g, yield: 24% over 2 steps). ¹H NMR (400 MHz, DMSO-d₆) δ 11.61 (br, 1H), 11.08 (s, 1H), 7.58 (dd, J=7.2, 8.8 Hz, 1H), 7.15 (d, J=8.8 Hz, 1H), 7.04 (d, J=7.2 Hz, 1H), 6.64 (s, 1H), 5.05 (dd, J=5.2, 12.8 Hz, 1H), 3.53 (t, J=6.4 Hz, 2H), 2.92-2.83 (m, 1H), 2.61-2.50 (m, 4H), 2.05-2.00 (m, 1H). MS (ESI) m/z=346.1 [M+H]⁺

### Example 15: 4-((2-(2,6-Dioxopiperidin-3-yl)-1,3-dioxoisoindolin-4-yl)amino)butanoic acid (Linker 15)

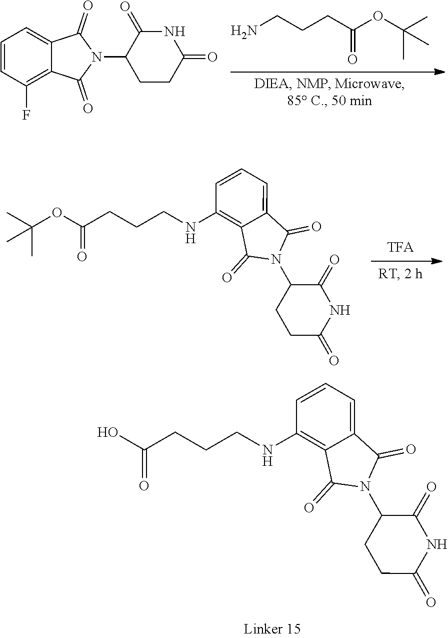

Linker 15 was synthesized following the same procedures as Linker 1 as described in Example 1. (1.27 g, yield: 13% over 2 steps). ¹H NMR (400 MHz, DMSO-d6) δ 12.12 (br, 1H), 11.08 (s, 1H), 7.58 (dd, J=7.2, 8.8 Hz, 1H), 7.13 (d, J=8.8 Hz, 1H), 7.03 (d, J=7.2 Hz, 1H), 6.64 (t, J=6.0 Hz, 1H), 5.05 (dd, J=5.6, 12.8 Hz, 1H), 3.33 (q, J=6.8 Hz, 2H), 2.93-2.83 (m, 1H), 2.61-2.50 (m, 2H), 2.31 (t, J=6.8 Hz, 2H), 2.07-2.00 (m, 1H), 1.83-1.75 (m, 2H). MS (ESI) m/z=360.1 [M+H]⁺

### Example 16: 5-((2-(2,6-Dioxopiperidin-3-yl)-1,3-dioxoisoindolin-4-yl)amino)pentanoic acid (Linker 16)

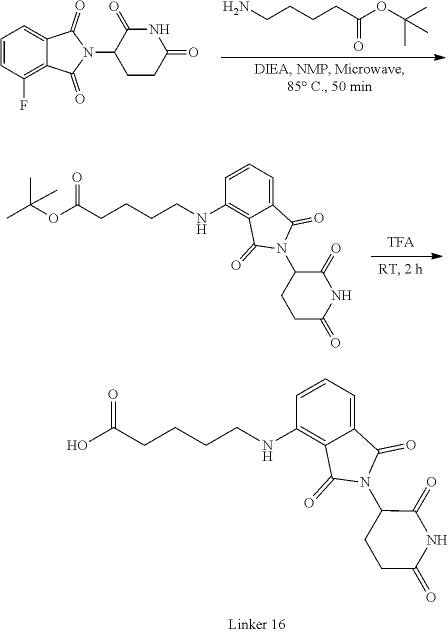

Linker 16 was synthesized following the same procedures as Linker 1 as described in Example 1. (1.4 g, yield: 15% over 2 steps). ¹H NMR (400 MHz, DMSO-d₆) δ12.02 (br, 1H), 11.08 (s, 1H), 7.58 (dd, J=8.8, 7.2 Hz, 1H), 7.10 (d, J=8.4 Hz, 1H), 7.02 (d, J=7.2 Hz, 1H), 6.64 (t, J=5.6 Hz, 1H), 5.07-5.03 (m, 1H), 3.32-3.02 (m, 2H), 2.93-2.84 (m, 1H), 2.61-2.54 (m, 2H), 2.28-2.25 (m, 2H), 2.05-2.01 (m, 1H), 1.60-1.51 (m, 4H). MS (ESI) m/z=374.1 [M+H]⁺

### Example 17: 6-((2-(2,6-Dioxopiperidin-3-yl)-1,3-dioxoisoindolin-4-yl)amino)hexanoic acid (Linker 17)

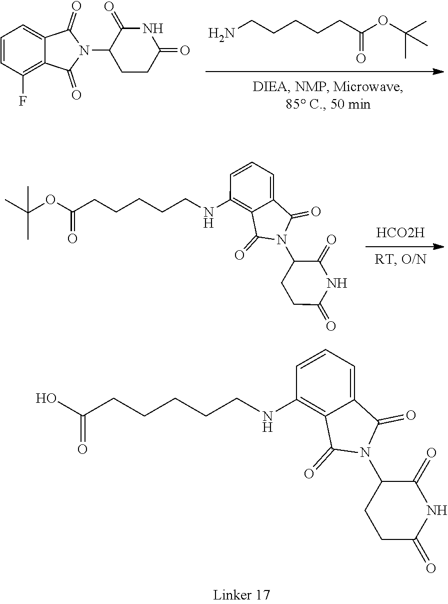

Linker 17 was synthesized following the same procedures as Linker 1 as described in Example 1. (1.43 g, yield: 18% over 2 steps). ¹H NMR (400 MHz, DMSO-d₆) δ 11.97 (s, 1H), 11.08 (s, 1H), 7.57 (dd, J=7.2, 8.8 Hz, 1H), 7.08 (d, J=8.8 Hz, 1H), 7.02 (d, J=7.2 Hz, 1H), 6.52 (t, J=6.0 Hz, 1H), 5.05 (dd, J=5.6, 12.8 Hz, 1H), 3.30 (q, J=6.8 Hz, 2H), 2.93-2.83 (m, 1H), 2.61-2.50 (m, 2H), 2.32 (t, J=7.2 Hz, 2H), 2.07-2.00 (m, 1H), 1.61-1.50 (m, 4H), 1.39-1.33 (m, 2H). MS (ESI) m/z=388.1 [M+H]⁺

### Example 18: 7-((2-(2,6-Dioxopiperidin-3-yl)-1,3-dioxoisoindolin-4-yl)amino)heptanoic acid (Linker 18)

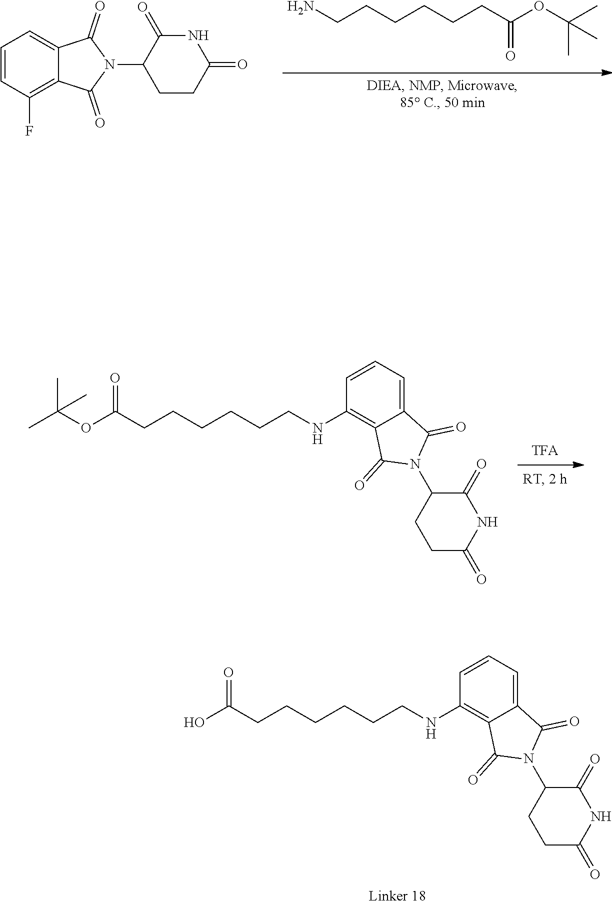 [SMILES: O=C1CCC(N2C(=O)c3cccc(F)c3C2=O)C(=O)N1 ; CC(C)(C)OC(=O)CCCCCCN ; CC(C)(C)OC(=O)CCCCCCNc1cccc2c1C(=O)N(C1CCC(=O)NC1=O)C2=O ; "TFA" ; O=C(O)CCCCCCNc1cccc2c1C(=O)N(C1CCC(=O)NC1=O)C2=O]

Linker 18 was synthesized following the same procedures as Linker 1 as described in Example 1. (2.3 g, yield: 24% over 2 steps). ¹H NMR (400 MHz, DMSO-d6) δ 11.92 (br, 1H), 11.08 (s, 1H), 7.57 (t, J=8.0 Hz, 1H), 7.13 (d, J=8.8 Hz, 1H), 7.03 (d, J=6.8 Hz, 1H), 6.52 (t, J=5.6 Hz, 1H), 5.05 (dd, J=5.6, 12.8 Hz, 1H), 3.30 (q, J=6.4 Hz, 2H), 2.93-2.83 (m, 1H), 2.61-2.50 (m, 2H), 2.31 (t, J=7.2 Hz, 2H), 2.07-2.00 (m, 1H), 1.58-1.48 (m, 4H), 1.34-1.31 (m, 4H). MS (ESI) m/z=402.1 [M+H]⁺

### Example 19: 8-((2-(2,6-Dioxopiperidin-3-yl)-1,3-dioxoisoindolin-4-yl)amino)octanoic acid (Linker 19)

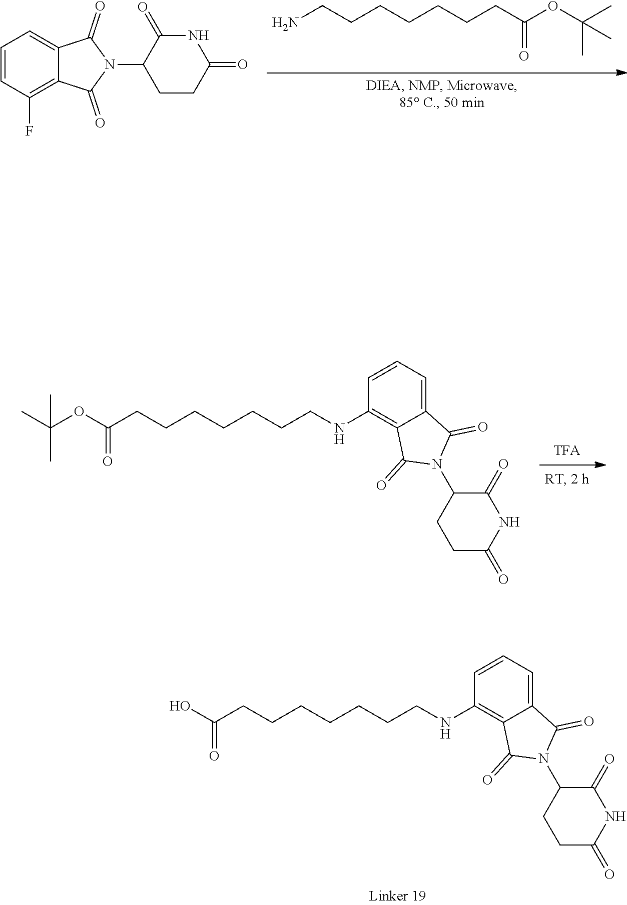 [SMILES: O=C1CCC(N2C(=O)c3cccc(F)c3C2=O)C(=O)N1 ; CC(C)(C)OC(=O)CCCCCCCN ; CC(C)(C)OC(=O)CCCCCCCNc1cccc2c1C(=O)N(C1CCC(=O)NC1=O)C2=O ; "TFA" ; O=C(O)CCCCCCCNc1cccc2c1C(=O)N(C1CCC(=O)NC1=O)C2=O]

Linker 19 was synthesized following the same procedures as Linker 1 as described in Example 1. (1.14 g, yield: 35% over 2 steps). ¹H NMR (400 MHz, DMSO-d6) δ 11.94 (s, 1H), 11.08 (s, 1H), 7.57 (t, J=8.0 Hz, 1H), 7.08 (d, J=8.4 Hz, 1H), 7.02 (d, J=6.8 Hz, 1H), 6.52 (t, J=5.6 Hz, 1H), 5.05 (dd, J=5.6, 12.8 Hz, 1H), 3.31-3.26 (m, 2H), 2.93-2.83 (m, 1H), 2.61-2.50 (m, 2H), 2.19 (t, J=7.2 Hz, 2H), 2.05-2.00 (m, 1H), 1.58-1.47 (m, 4H), 1.35-1.25 (s, 6H). MS (ESI) m/z=416.1 [M+H]⁺

### Example 20: 3-(2-((2-(2,6-Dioxopiperidin-3-yl)-1,3-dioxoisoindolin-4-yl)amino)ethoxy) propanoic acid (Linker 20)

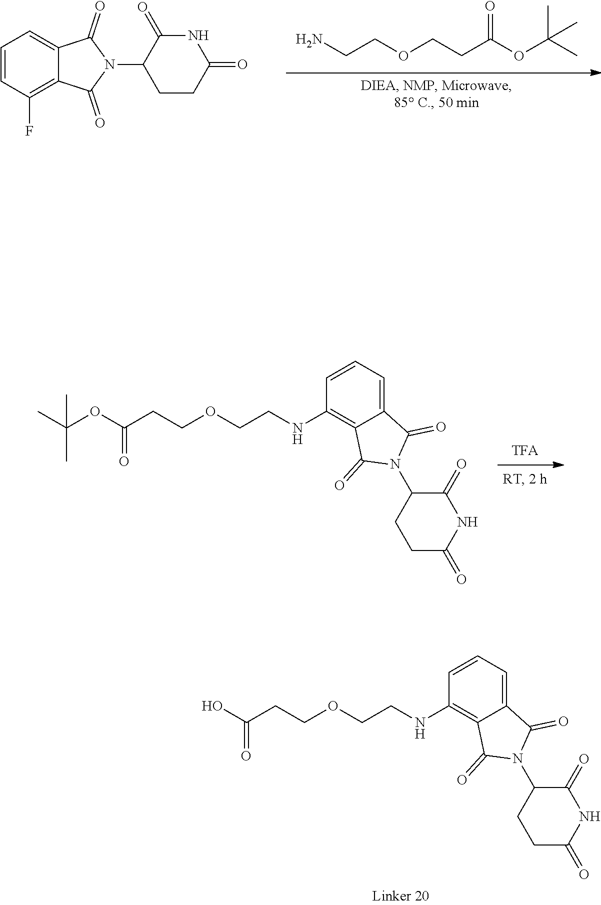 [SMILES: O=C1CCC(N2C(=O)c3cccc(F)c3C2=O)C(=O)N1 ; CC(C)(C)OC(=O)CCOCCN ; CC(C)(C)OC(=O)CCOCCNc1cccc2c1C(=O)N(C1CCC(=O)NC1=O)C2=O ; "TFA" ; O=C(O)CCOCCNc1cccc2c1C(=O)N(C1CCC(=O)NC1=O)C2=O]

Linker 20 was synthesized following the same procedures as Linker 1 as described in Example 1. (3.5 g, yield: 18% over 2 steps). ¹H NMR (400 MHz, DMSO-d6) δ 12.18 (s, 1H), 11.08 (s, 1H), 7.58 (dd, J=7.2 Hz, 8.8 Hz, 1H), 7.13 (d, J=8.4 Hz, 1H), 7.04 (d, J=7.2 Hz, 1H), 6.58 (t, J=5.6 Hz 1H), 5.05 (dd, J=6.4 Hz, 12.8 Hz, 1H), 3.67-3.58 (m, 4H), 3.47-3.43 (m, 2H), 2.93-2.84 (m, 1H), 2.61-2.45 (m, 4H), 2.07-2.01 (m, 1H). MS (ESI) m/z=390.1 [M+H]⁺

### Example 21: 3-(2-(2-((2-(2,6-Dioxopiperidin-3-yl)-1,3-dioxoisoindolin-4-yl)amino)ethoxy)ethoxy)propanoic acid (Linker 21)

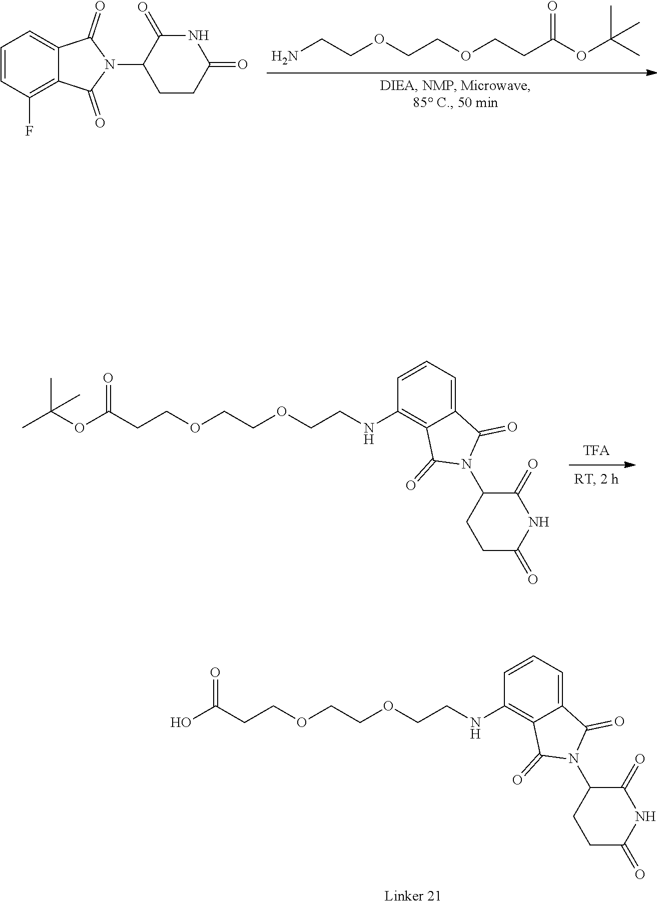 [SMILES: O=C1CCC(N2C(=O)c3cccc(F)c3C2=O)C(=O)N1 ; CC(C)(C)OC(=O)CCOCCOCCN ; CC(C)(C)OC(=O)CCOCCOCCNc1cccc2c1C(=O)N(C1CCC(=O)NC1=O)C2=O ; "TFA" ; O=C(O)CCOCCOCCNc1cccc2c1C(=O)N(C1CCC(=O)NC1=O)C2=O]

Linker 21 was synthesized following the same procedures as Linker 1 as described in Example 1. (2.0 g, yield: 24% over 2 steps). ¹H NMR (400 MHz, DMSO-d6) δ 12.13 (s, 1H), 11.08 (s, 1H), 7.58 (dd, J=7.2 Hz, 8.4 Hz, 1H), 7.14 (d, J=8.4 Hz, 1H), 7.04 (d, J=6.8 Hz, 1H), 6.60 (t, J=6.0 Hz 1H), 5.05 (dd, J=5.2 Hz, 12.4 Hz, 1H), 3.63-3.44 (m, 10H), 2.88-2.85 (m, 1H), 2.61-2.49 (m, 2H), 2.44-2.41 (m, 2H), 2.04-2.01 (m, 1H). MS (ESI) m/z=434.1 [M+H]⁺

### Example 22: 3-(2-(2-(2-((2-(2,6-Dioxopiperidin-3-yl)-1,3-dioxoisoindolin-4-yl)amino)ethoxy)ethoxy)ethoxy)propanoic acid (Linker 22)

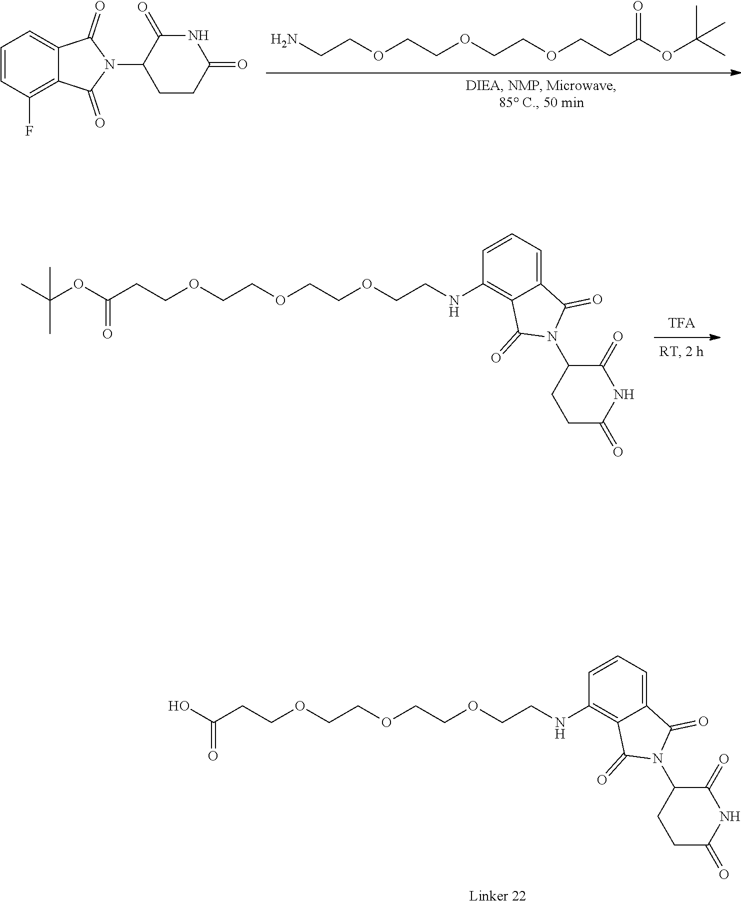 [SMILES: O=C1CCC(N2C(=O)c3cccc(F)c3C2=O)C(=O)N1 ; CC(C)(C)OC(=O)CCOCCOCCOCCN ; CC(C)(C)OC(=O)CCOCCOCCOCCNc1cccc2c1C(=O)N(C1CCC(=O)NC1=O)C2=O ; "TFA" ; O=C(O)CCOCCOCCOCCNc1cccc2c1C(=O)N(C1CCC(=O)NC1=O)C2=O]

Linker 22 was synthesized following the same procedures as Linker 1 as described in Example 1. (3.2 g, yield: 42% over 2 steps). ¹H NMR (400 MHz, DMSO-d6) δ 12.14 (s, 1H), 11.08 (s, 1H), 7.58 (dd, J=7.2 Hz, 8.4 Hz, 1H), 7.14 (d, J=8.8 Hz, 1H), 7.04 (d, J=6.8 Hz, 1H), 6.60 (t, J=6.0 Hz, 1H), 5.05 (dd, J=5.2 Hz, 12.8 Hz, 1H), 3.63-3.45 (m, 14H), 2.88-2.85 (m, 1H), 2.61-2.49 (m, 2H), 2.44-2.40 (m, 2H), 2.04-2.01 (m, 1H). MS (ESI) m/z=478.2 [M+H]⁺

### Example 23: 1-((2-(2,6-Dioxopiperidin-3-yl)-1,3-dioxoisoindolin-4-yl)amino)-3,6,9,12-tetraoxapentadecan-15-oic acid (Linker 23)

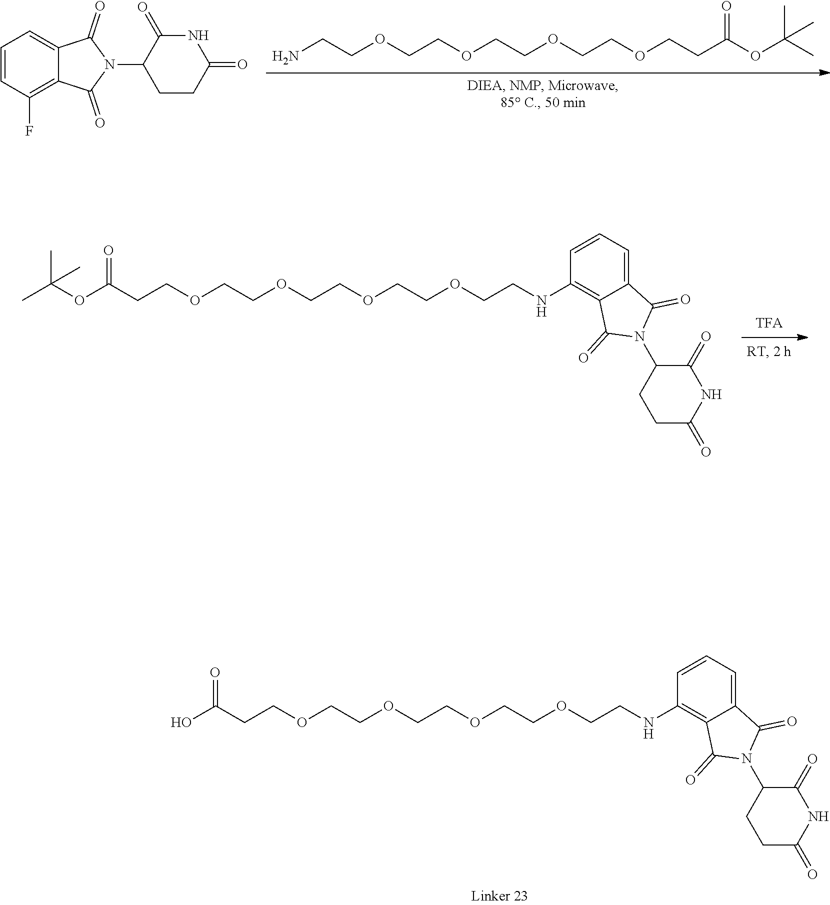

Linker 23 was synthesized following the same procedures as Linker 1 as described in Example 1. (2.3 g, yield: 31% over 2 steps). ¹H NMR (400 MHz, DMSO-d6) δ 12.14 (s, 1H), 11.08 (s, 1H), 7.58 (dd, J=7.2 Hz, 8.8 Hz, 1H), 7.14 (d, J=8.4 Hz, 1H), 7.04 (d, J=7.2 Hz, 1H), 6.60 (t, J=6.0 Hz, 1H), 5.05 (dd, J=5.2 Hz, 12.8 Hz, 1H), 3.63-3.48 (m, 18H), 2.898-2.85 (m, 1H), 2.61-2.49 (m, 2H), 2.44-2.41 (m, 2H), 2.04-2.01 (m, 1H). MS (ESI) m/z=522.2 [M+H]⁺

### Example 24: 1-((2-(2,6-Dioxopiperidin-3-yl)-1,3-dioxoisoindolin-4-yl)amino)-3,6,9,12,15-pentaoxaoctadecan-18-oic acid (Linker 24)

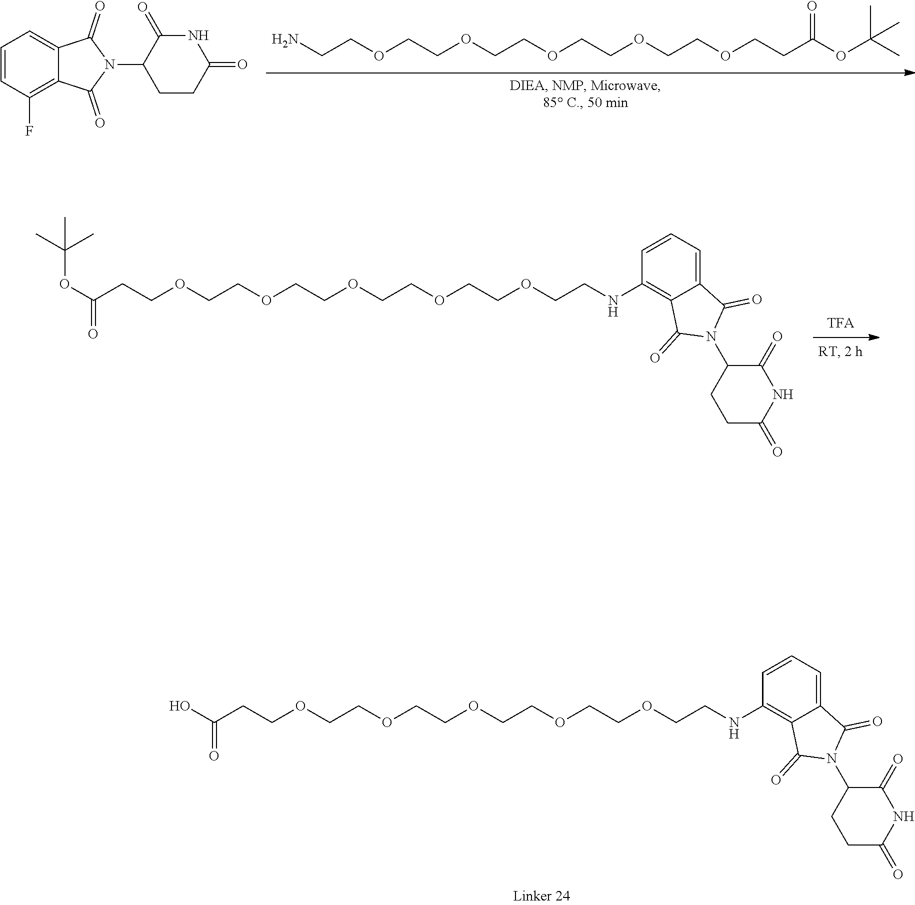

Linker 24 was synthesized following the same procedures as Linker 1 as described in Example 1. (2.4 g, yield: 36% over 2 steps). ¹H NMR (400 MHz, DMSO-d6) δ 11.09 (s, 1H), 7.58 (dd, J=7.2, 8.4 Hz, 1H), 7.13 (d, J=8.4 Hz, 1H), 7.04 (d, J=7.2 Hz, 1H), 6.60 (t, J=5.6 Hz, 1H), 5.05 (dd, J=5.6, 12.8 Hz, 1H), 3.64-3.46 (m, 22H), 2.93-2.83 (m, 1H), 2.61-2.50 (m, 2H), 2.44-2.40 (m, 2H), 2.02 (t, J=6.4 Hz, 1H). MS (ESI) m/z=566.2 [M+H]⁺

### Example 25: (2S,4R)-1-((S)-2-(2-Aminoacetamido)-3,3-dimethylbutanoyl)-4-hydroxy-N-(4-(4-methylthiazol-5-yl)benzyl)pyrrolidine-2-carboxamide (Linker 25)

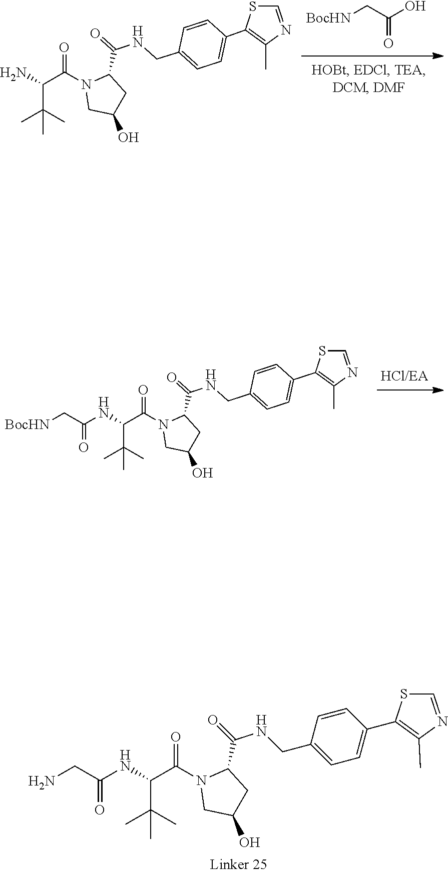 [SMILES: Cc1ncsc1-c1ccc(CNC(=O)[C@@H]2C[C@@H](O)CN2C(=O)[C@@H](N)C(C)(C)C)cc1 ; CC(C)(C)OC(=O)NCC(=O)O ; Cc1ncsc1-c1ccc(CNC(=O)[C@@H]2C[C@@H](O)CN2C(=O)[C@@H](NC(=O)CNC(=O)OC(C)(C)C)C(C)(C)C)cc1 ; "HCl/EA" ; Cc1ncsc1-c1ccc(CNC(=O)[C@@H]2C[C@@H](O)CN2C(=O)[C@@H](NC(=O)CN)C(C)(C)C)cc1]

### Step 1

To a solution of (2S,4R)-1-((S)-2-amino-3,3-dimethylbutanoyl)-4-hydroxy-N-(4-(4-methylthiazol-5-yl)benzyl)pyrrolidine-2-carboxamide (2.00 g, 4.67 mmol), 2-((tert-butoxycarbonyl)amino) acetic acid (900 mg, 5.14 mmol) and triethylamine (TEA) (3.2 mL, 23.35 mmol) in DCM/DMF (225 mL/11 mL) was added EDCI (1.07 g, 5.60 mmol), HOBt (756 mg, 5.60 mmol) at 0° C. The mixture was stirred at room temperature for 16 hours. The mixture was poured into water and extracted with DCM. The combined organic layers were concentrated and the residue was purified by chromatography on a silica gel column (DCM/MeOH=20/1, v/v) to give the desired product tert-butyl (2-(((S)-1-((2S,4R)-4-hydroxy-2-((4-(4-methylthiazol-5-yl)benzyl)carbamoyl)pyrrolidin-1-yl)-3,3-dimethyl-1-oxobutan-2-yl)amino)-2-oxoethyl)carbamate (1.5 g, yield: 55%). MS (ESI) m/z=588.2 [M+H]⁺

### Step 2

To a solution of tert-butyl (2-(((S)-1-((2S,4R)-4-hydroxy-2-((4-(4-methylthiazol-5-yl) benzyl)carbamoyl)pyrrolidin-1-yl)-3,3-dimethyl-1-oxobutan-2-yl)amino)-2-oxoethyl)carbamate (1.50 g, 2.56 mmol) in ethylacetate (EA) (30 mL) was added HCl/EA (100 mL). The mixture was stirred at room temperature for 3 hours and filtered to give the desired product which was dissolved in water (100 mL) and lyophilized to give (2S,4R)-1-((S)-2-(2-aminoacetamido)-3,3-dimethylbutanoyl)-4-hydroxy-N-(4-(4-methylthiazol-5-yl)benzyl)pyrrolidine-2-carboxamide hydrochloride (Linker 25) (1.07 g, yield: 80%). ¹H NMR (400 MHz, DMSO-d₆) 9.29 (s, 1H), 8.72 (s, 1H), 8.56 (d, J=9.2 Hz, 1H), 8.26 (s, 3H), 7.38-7.47 (m, 4H), 4.61 (d, J=9.2 Hz, 1H), 4.36-4.47 (m, 3H), 4.20-4.25 (m, 1H), 3.60-3.70 (m, 4H), 2.46 (s, 3H), 2.10-2.05 (m, 1H), 1.97-1.89 (m, 1H), 0.95 (s, 9H). MS (ESI) m/z=488.3 [M+H]⁺

### Example 26: (2S,4R)-1-((S)-2-(3-Aminopropanamido)-3,3-dimethylbutanoyl)-4-hydroxy-N-(4-(4-methylthiazol-5-yl)benzyl)pyrrolidine-2-carboxamide (Linker 26)

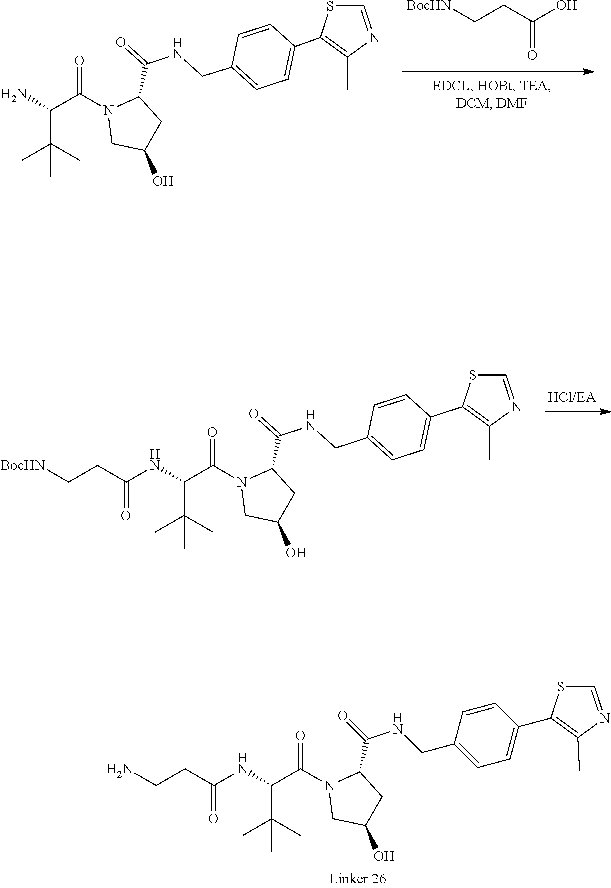 [SMILES: Cc1ncsc1-c1ccc(CNC(=O)[C@@H]2C[C@@H](O)CN2C(=O)[C@@H](N)C(C)(C)C)cc1 ; CC(C)(C)OC(=O)NCCC(=O)O ; Cc1ncsc1-c1ccc(CNC(=O)[C@@H]2C[C@@H](O)CN2C(=O)[C@@H](NC(=O)CCNC(=O)OC(C)(C)C)C(C)(C)C)cc1 ; "HCl/EA" ; Cc1ncsc1-c1ccc(CNC(=O)[C@@H]2C[C@@H](O)CN2C(=O)[C@@H](NC(=O)CCN)C(C)(C)C)cc1]

Linker 26 was synthesized following the same procedures as Linker 25 as described in Example 25. (1.38 g, yield: 37% over 2 steps). ¹H NMR (400 MHz, DMSO-d₆) 9.36 (s, 1H), 8.68 (s, 1H), 8.26 (d, J=9.2 Hz, 1H), 8.16 (s, 3H), 7.49-7.39 (m, 4H), 4.53 (d, J=9.2 Hz, 1H), 4.47-4.35 (m, 3H), 4.24-4.19 (m, 1H), 3.69-3.60 (m, 2H), 2.94-2.93 (m, 2H), 2.64 (t, J=7.2 Hz, 2H), 2.48 (s, 3H), 2.06-2.01 (m, 1H), 1.92-1.85 (m, 1H), 0.95 (s, 9H). MS (ESI) m/z=502.3 [M+H]⁺

### Example 27: (2S,4R)-1-((S)-2-(4-Aminobutanamido)-3,3-dimethylbutanoyl)-4-hydroxy-N-(4-(4-methylthiazol-5-yl)benzyl)pyrrolidine-2-carboxamide (Linker 27)

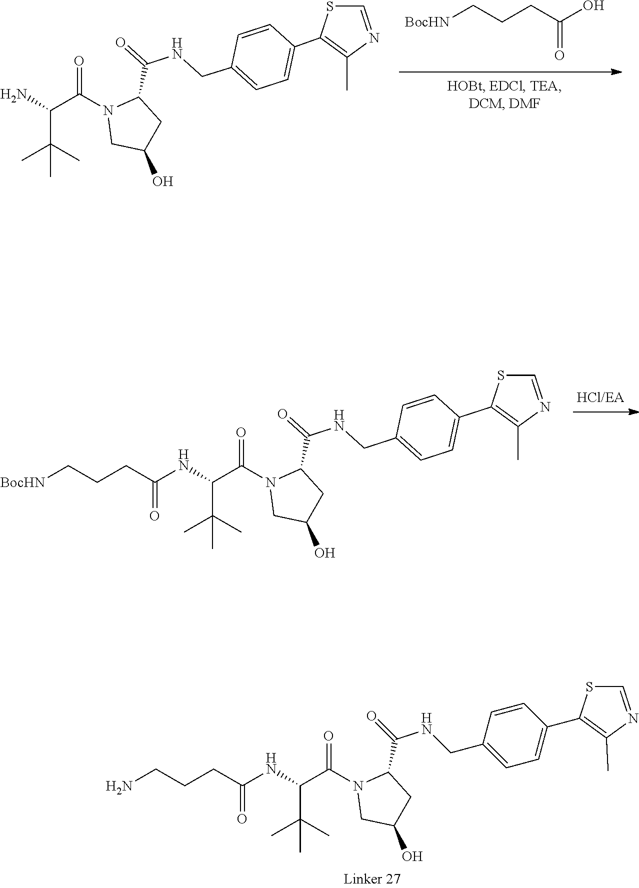 [SMILES: Cc1ncsc1-c1ccc(CNC(=O)[C@@H]2C[C@@H](O)CN2C(=O)[C@@H](N)C(C)(C)C)cc1 ; CC(C)(C)OC(=O)NCCCC(=O)O ; Cc1ncsc1-c1ccc(CNC(=O)[C@@H]2C[C@@H](O)CN2C(=O)[C@@H](NC(=O)CCCNC(=O)OC(C)(C)C)C(C)(C)C)cc1 ; "HCl/EA" ; Cc1ncsc1-c1ccc(CNC(=O)[C@@H]2C[C@@H](O)CN2C(=O)[C@@H](NC(=O)CCCN)C(C)(C)C)cc1]

Linker 27 was synthesized following the same procedures as Linker 25 as described in Example 25. (1.38 g, yield: 46% over 2 steps). ¹H NMR (400 MHz, DMSO-d₆) 9.66 (s, 1H), 8.74 (t, J=6.0, 1H), 8.25 (s, 3H), 8.03 (d, J=9.2 Hz, 1H), 7.49-7.41 (m, 4H), 4.53 (d, J=9.2 Hz, 1H), 4.51-4.36-4.35 (m, 3H), 4.29-4.24 (m, 1H), 3.71-3.65 (m, 2H), 2.79-2.77 (m, 2H), 2.52 (s, 3H), 2.45-2.27 (m, 2H), 2.12-2.07 (m, 1H), 1.94-1.80 (m, 3H), 0.94 (s, 9H). MS (ESI) m/z=516.0 [M+H]⁺.

### Example 28: (2S,4R)-1-((S)-2-(5-Aminopentanamido)-3,3-dimethylbutanoyl)-4-hydroxy-N-(4-(4-methylthiazol-5-yl)benzyl)pyrrolidine-2-carboxamide (Linker 28)

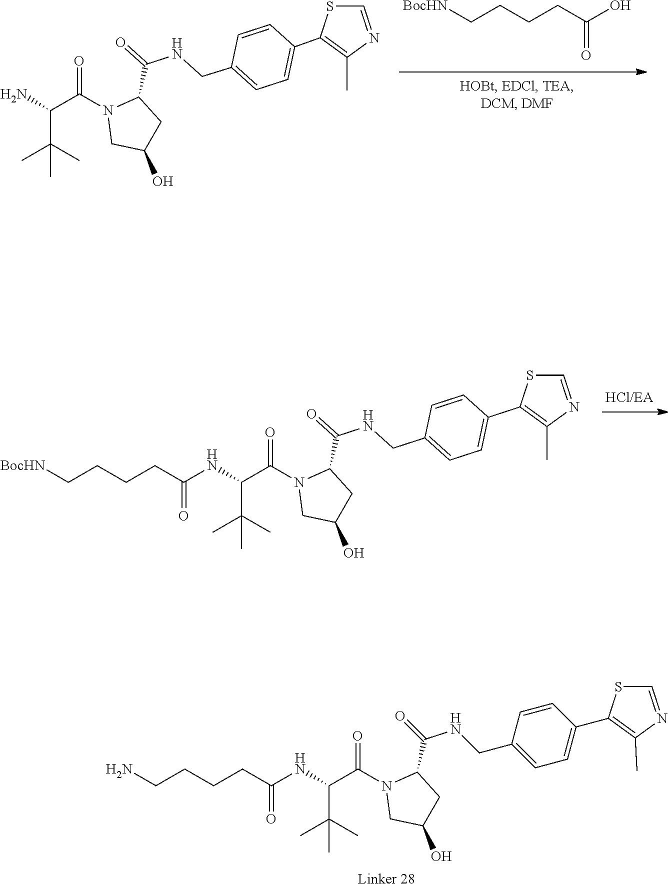 [SMILES: Cc1ncsc1-c1ccc(CNC(=O)[C@@H]2C[C@@H](O)CN2C(=O)[C@@H](N)C(C)(C)C)cc1 ; CC(C)(C)OC(=O)NCCCCC(=O)O ; Cc1ncsc1-c1ccc(CNC(=O)[C@@H]2C[C@@H](O)CN2C(=O)[C@@H](NC(=O)CCCCNC(=O)OC(C)(C)C)C(C)(C)C)cc1 ; "HCl/EA" ; Cc1ncsc1-c1ccc(CNC(=O)[C@@H]2C[C@@H](O)CN2C(=O)[C@@H](NC(=O)CCCCN)C(C)(C)C)cc1]

Linker 28 was synthesized following the same procedures as Linker 25 as described in Example 25. (1.50 g, yield: 57% over 2 steps). ¹H NMR (400 MHz, DMSO-d₆) 9.52 (s, 1H), 8.73 (t, J=11.6 Hz, 1H), 8.20 (s, 3H), 7.95 (d, J=9.6 Hz, 1H), 7.43-7.50 (m, 4H), 4.55 (d, J=9.2 Hz, 1H), 4.38-4.50 (m, 3H), 4.23-4.29 (m, 1H), 3.64-3.71 (m, 2H), 2.74-2.78 (m, 2H), 2.51 (s, 3H), 2.30-2.35 (m, 1H), 2.18-2.23 (m, 1H), 2.07-2.12 (m, 1H), 1.88-1.95 (m, 1H), 1.58 (d, J=4.4 Hz, 4H), 0.96 (s, 9H). MS (ESI) m/z=530.1 [M+H]⁺

### Example 29: (2S,4R)-1-((S)-2-(6-Aminohexanamido)-3,3-dimethylbutanoyl)-4-hydroxy-N-(4-(4-methylthiazol-5-yl)benzyl)pyrrolidine-2-carboxamide (Linker 29)

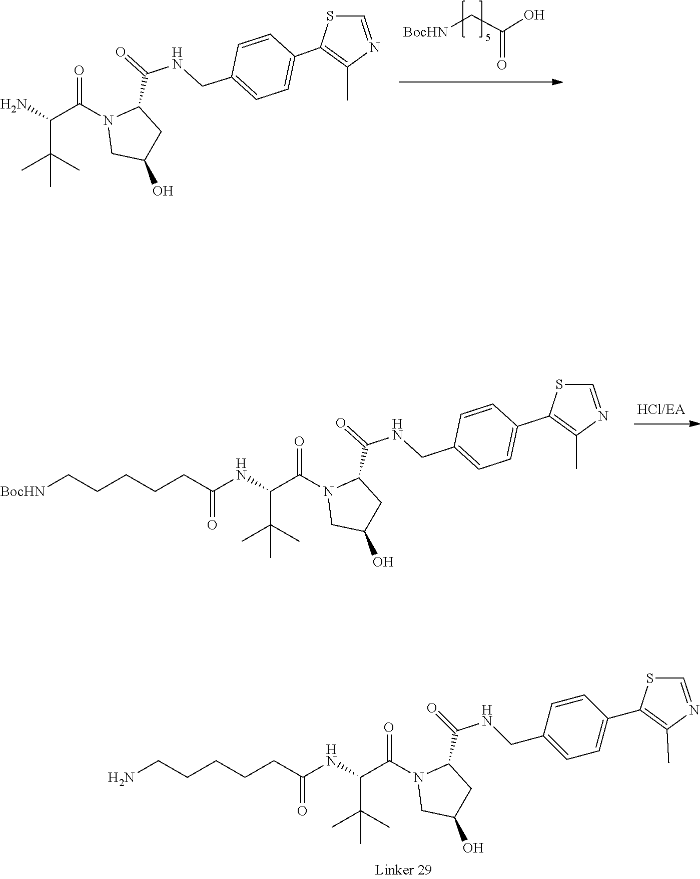

Linker 29 was synthesized following the same procedures as Linker 25 as described in Example 25. (2.70 g, yield: 87% over 2 steps). ¹H NMR (400 MHz, DMSO-d6): 9.36 (s, 1H), 8.69 (t, J=6.4 Hz, 1H), 8.12 (brs, 3H), 7.92 (d, J=9.6 Hz, 1H), 7.44 (dd, J=13.6, 8.4 Hz, 4H), 4.54 (d, J=9.6 Hz, 1H), 4.48-4.39 (m, 2H), 4.36 (brs, 1H), 4.28-4.19 (m, 1H), 3.72-3.60 (m, 2H), 2.79-2.67 (m, 2H), 2.49 (s, 3H), 2.31-2.21 (m, 1H), 2.20-2.12 (m, 1H), 2.10-2.01 (m, 1H), 1.94-1.85 (m, 1H), 1.62-1.54 (m, 2H), 1.53-1.44 (m, 2H), 1.34-1.22 (m, 2H), 0.94 (s, 9H). MS (ESI) m/z=544.3 [M+H]⁺.

### Example 30: (2S,4R)-1-((S)-2-(7-Aminoheptanamido)-3,3-dimethylbutanoyl)-4-hydroxy-N-(4-(4-methylthiazol-5-yl)benzyl)pyrrolidine-2-carboxamide (Linker 30)

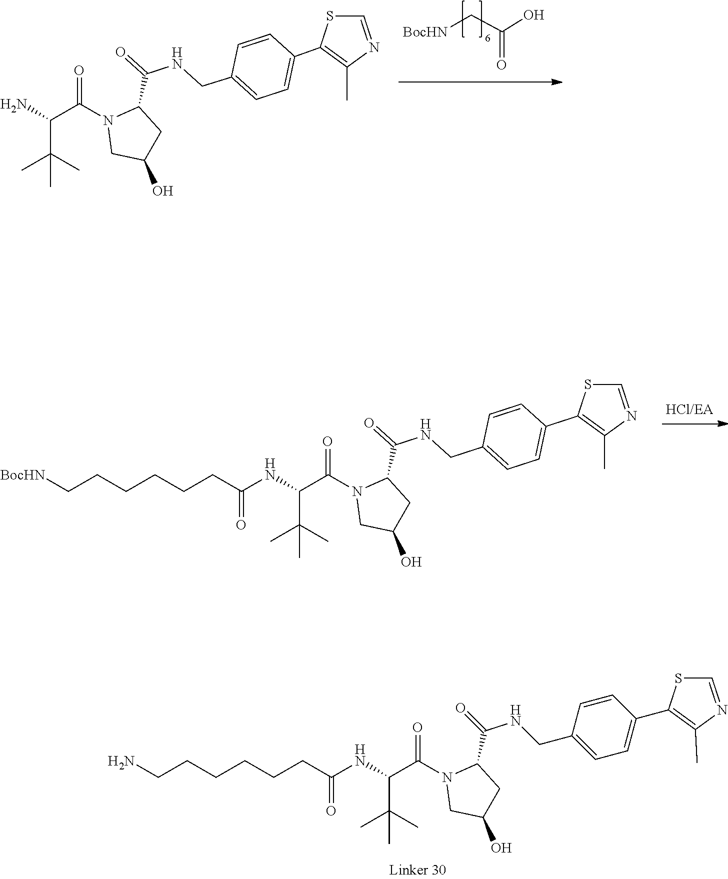

Linker 30 was synthesized following the same procedures as Linker 25 as described in Example 25. (2.13 g, yield: 76% over 2 steps). ¹H NMR (400 MHz, DMSO-d6): 9.45 (s, 1H), 8.70 (t, J=6.0 Hz, 1H), 8.14 (brs, 3H), 7.86 (d, J=9.2 Hz, 1H), 7.44 (dd, J=12.8, 8.4 Hz, 4H), 4.54 (d, J=9.2 Hz, 1H), 4.49-4.40 (m, 2H), 4.36 (brs, 1H), 4.29-4.20 (m, 1H), 3.71-3.61 (m, 2H), 2.78-2.67 (m, 2H), 2.50 (s, 3H), 2.31-2.22 (m, 1H), 2.21-2.13 (m, 1H), 2.11-2.03 (m, 1H), 1.95-1.85 (m, 1H), 1.60-1.44 (m, 4H), 1.35-1.18 (m, 4H), 0.94 (s, 9H). MS (ESI) m/z=558.3 [M+H]⁺.

### Example 31: (2S,4R)-1-((S)-2-(8-Aminooctanamido)-3,3-dimethylbutanoyl)-4-hydroxy-N-(4-(4-methylthiazol-5-yl)benzyl)pyrrolidine-2-carboxamide (Linker 31)

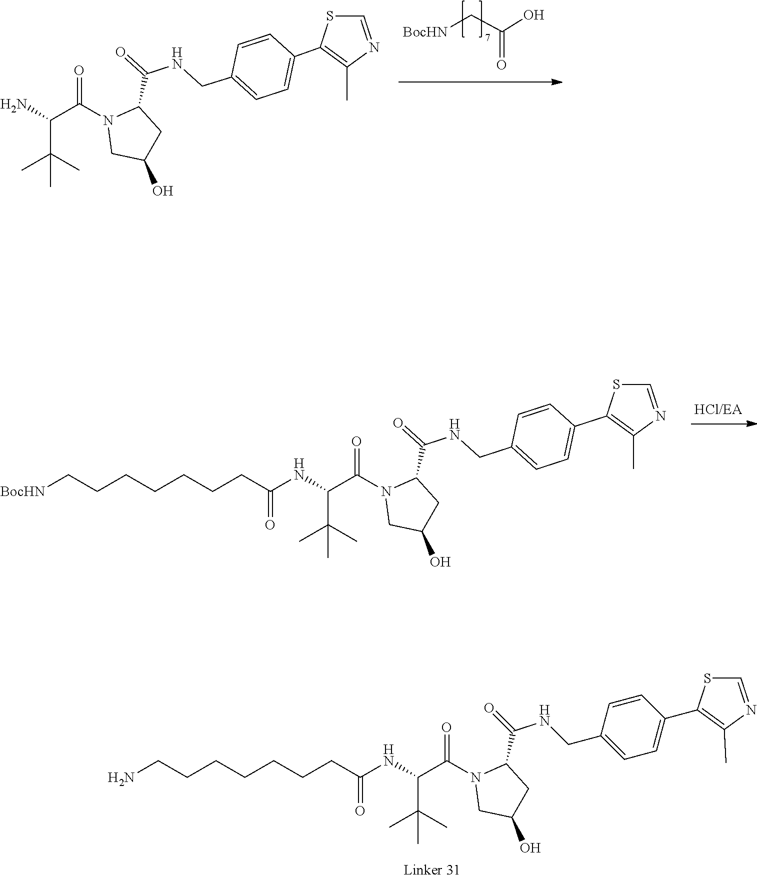

Linker 31 was synthesized following the same procedures as Linker 25 as described in Example 25. (1.81 g, yield: 65% over 2 steps). ¹H NMR (400 MHz, DMSO-d6): 9.35 (s, 1H), 8.69 (t, J=6.0 Hz, 1H), 8.11 (brs, 3H), 7.88 (d, J=9.2 Hz, 1H), 7.44 (dd, J=14.0, 8.4 Hz, 4H), 4.54 (d, J=9.6 Hz, 1H), 4.48-4.39 (m, 2H), 4.36 (brs, 1H), 4.27-4.20 (m, 1H), 3.71-3.60 (m, 2H), 2.78-2.68 (m, 2H), 2.49 (s, 3H), 2.31-2.22 (m, 1H), 2.18-2.11 (m, 1H), 2.09-2.01 (m, 1H), 1.94-1.85 (m, 1H), 1.58-1.44 (m, 4H), 1.32-1.19 (m, 6H), 0.94 (s, 9H). MS (ESI) m/z=572.3 [M+H]⁺.

### Example 32: (2S,4R)-1-((S)-2-(9-Aminononanamido)-3,3-dimethylbutanoyl)-4-hydroxy-N-(4-(4-methylthiazol-5-yl)benzyl)pyrrolidine-2-carboxamide (Linker 32)

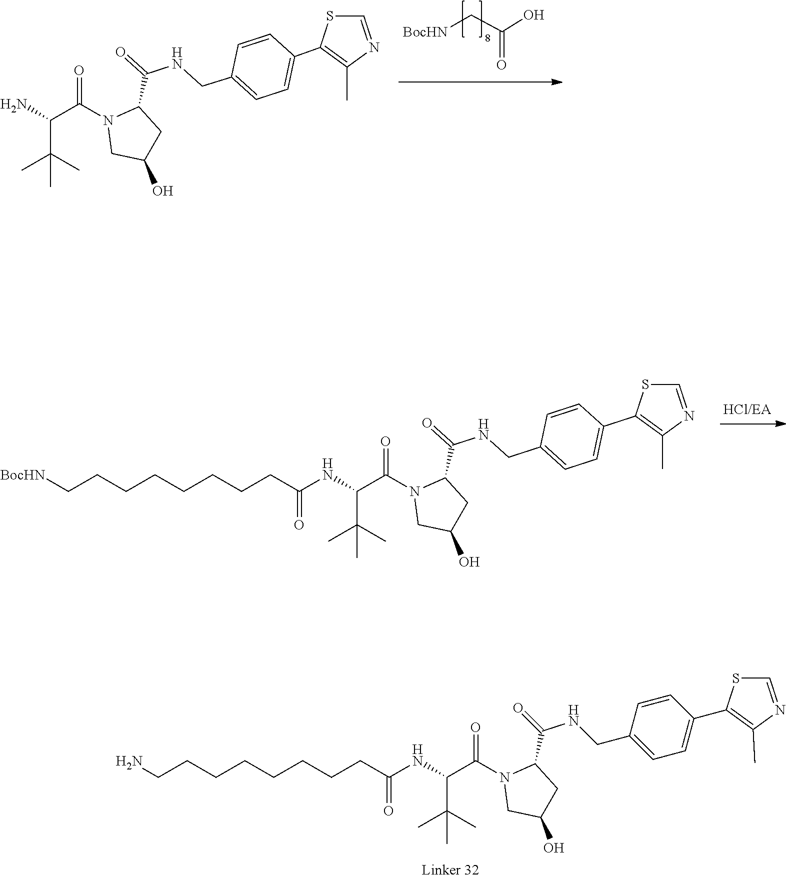

Linker 32 was synthesized following the same procedures as Linker 25 as described in Example 25. (2.32 g, yield: 80% over 2 steps). ¹H NMR (400 MHz, DMSO-d6): 9.30 (s, 1H), 8.67 (t, J=6.4 Hz, 1H), 8.10 (brs, 3H), 7.88 (d, J=9.2 Hz, 1H), 7.43 (dd, J=14.0, 8.8 Hz, 4H), 4.55 (d, J=9.2 Hz, 1H), 4.48-4.39 (m, 2H), 4.35 (brs, 1H), 4.28-4.19 (m, 1H), 3.71-3.60 (m, 2H), 2.77-2.67 (m, 2H), 2.48 (s, 3H), 2.31-2.22 (m, 1H), 2.17-2.10 (m, 1H), 2.09-2.01 (m, 1H), 1.94-1.85 (m, 1H), 1.60-1.40 (m, 4H), 1.33-1.19 (m, 8H), 0.94 (s, 9H), m/z=586.3 [M+H]⁺.

### Example 33: (2S,4R)-1-((S)-2-(10-Aminodecanamido)-3,3-dimethylbutanoyl)-4-hydroxy-N-(4-(4-methylthiazol-5-yl)benzyl)pyrrolidine-2-carboxamide (Linker 33)

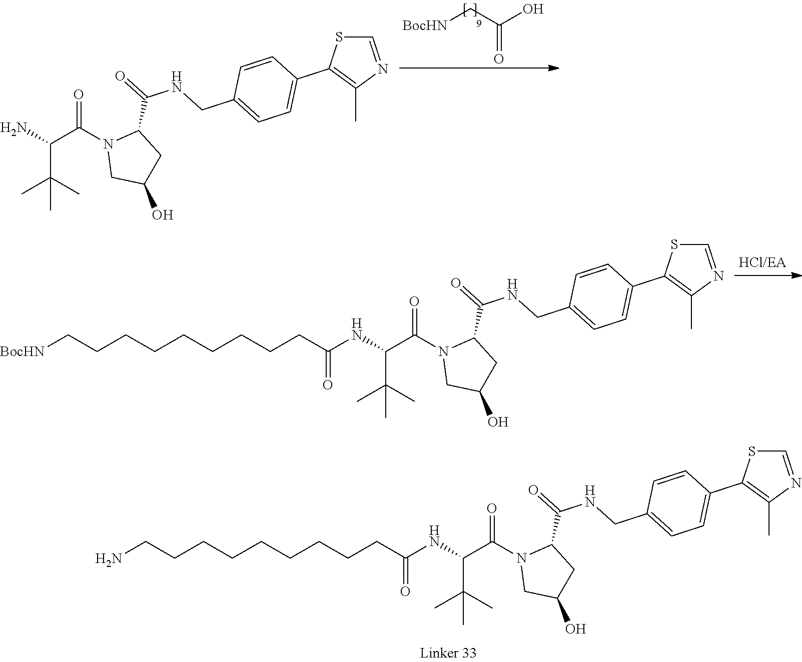 [SMILES: Cc1ncsc1-c1ccc(CNC(=O)[C@@H]2C[C@@H](O)CN2C(=O)[C@@H](N)C(C)(C)C)cc1 ; CC(C)(C)OC(=O)NCC(=O)O ; Cc1ncsc1-c1ccc(CNC(=O)[C@@H]2C[C@@H](O)CN2C(=O)[C@@H](NC(=O)CCCCCCCCCNC(=O)OC(C)(C)C)C(C)(C)C)cc1 ; Cc1ncsc1-c1ccc(CNC(=O)[C@@H]2C[C@@H](O)CN2C(=O)[C@@H](NC(=O)CCCCCCCCCN)C(C)(C)C)cc1]

Linker 33 was synthesized following the same procedures as Linker 25 as described in Example 25. (2.29 g, yield: 77% over 2 steps). ¹H NMR (400 MHz, DMSO-d6): 9.41 (s, 1H), 8.67 (t, J=6.0 Hz, 1H), 8.14 (brs, 3H), 7.85 (d, J=8.8 Hz, 1H), 7.44 (dd, J=13.6, 8.8 Hz, 4H), 4.54 (d, J=8.8 Hz, 1H), 4.48-4.39 (m, 2H), 4.36 (brs, 1H), 4.29-4.20 (m, 1H), 3.71-3.60 (m, 2H), 2.78-2.67 (m, 2H), 2.49 (s, 3H), 2.32-2.22 (m, 1H), 2.17-2.11 (m, 1H), 2.10-2.01 (m, 1H), 1.95-1.86 (m, 1H), 1.62-1.40 (m, 4H), 1.34-1.16 (m, 10H), 0.94 (s, 9H). MS (ESI) m/z=600.4 [M+H]⁺.

### Example 34: (2S,4R)-1-((S)-2-(11-Aminoundecanamido)-3,3-dimethylbutanoyl)-4-hydroxy-N-(4-(4-methylthiazol-5-yl)benzyl)pyrrolidine-2-carboxamide (Linker 34)

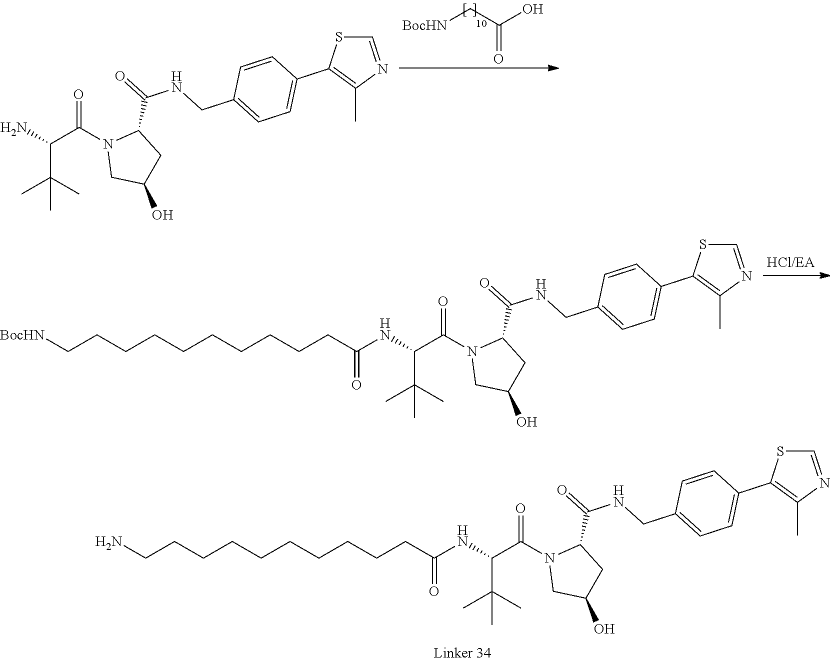 [SMILES: Cc1ncsc1-c1ccc(CNC(=O)[C@@H]2C[C@@H](O)CN2C(=O)[C@@H](N)C(C)(C)C)cc1 ; CC(C)(C)OC(=O)NCC(=O)O ; Cc1ncsc1-c1ccc(CNC(=O)[C@@H]2C[C@@H](O)CN2C(=O)[C@@H](NC(=O)CCCCCCCCCCNC(=O)OC(C)(C)C)C(C)(C)C)cc1 ; Cc1ncsc1-c1ccc(CNC(=O)[C@@H]2C[C@@H](O)CN2C(=O)[C@@H](NC(=O)CCCCCCCCCCN)C(C)(C)C)cc1]

Linker 34 was synthesized following the same procedures as Linker 25 as described in Example 25. (1.10 g, yield: 37% over 2 steps). ¹H NMR (400 MHz, DMSO-d6): 8.99 (s, 1H), 8.61 (t, J=6.4 Hz, 1H), 7.87 (d, J=8.8 Hz, 1H), 7.41 (dd, J=17.6, 8.0 Hz, 4H), 4.55 (d, J=9.6 Hz, 1H), 4.49-4.40 (m, 2H), 4.36 (brs, 1H), 4.26-4.17 (m, 1H), 3.70-3.64 (m, 2H), 2.59-2.52 (m, 2H), 2.45 (s, 3H), 2.31-2.22 (m, 1H), 2.16-2.08 (m, 1H), 2.06-1.99 (m, 1H), 1.96-1.86 (m, 1H), 1.56-1.42 (m, 2H), 1.39-1.30 (m, 2H), 1.28-1.19 (m, 12H), 0.94 (s, 9H). MS (ESI) m/z=614.4 [M+H]⁺.

### Example 35: (2S,4R)-14(S)-2-(2-(2-Aminoethoxy)acetamido)-3,3-dimethylbutanoyl)-4-hydroxy-N-(4-(4-methylthiazol-5-yl)benzyl)pyrrolidine-2-carboxamide (Linker 35)

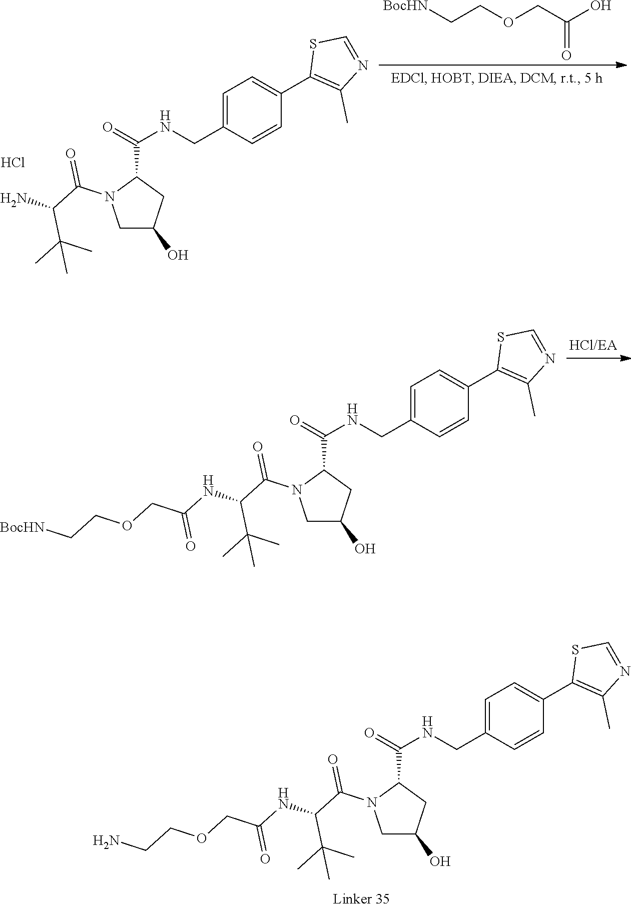 [SMILES: Cl ; Cc1ncsc1-c1ccc(CNC(=O)[C@@H]2C[C@@H](O)CN2C(=O)[C@@H](N)C(C)(C)C)cc1 ; CC(C)(C)OC(=O)NCCOCC(=O)O ; Cc1ncsc1-c1ccc(CNC(=O)[C@@H]2C[C@@H](O)CN2C(=O)[C@@H](NC(=O)COCCNC(=O)OC(C)(C)C)C(C)(C)C)cc1 ; Cc1ncsc1-c1ccc(CNC(=O)[C@@H]2C[C@@H](O)CN2C(=O)[C@@H](NC(=O)COCCN)C(C)(C)C)cc1]

Linker 35 was synthesized following the same procedures as Linker 25 as described in Example 25. (1.35 g, yield: 55% over 2 steps). ¹H NMR (400 MHz, DMSO-d₆) 9.23 (s, 1H), 8.70 (t, J=6.0 Hz, 1H), 8.35-8.14 (m, 3H), 7.78 (d, J=9.6 Hz, 1H), 7.47-7.38 (m, 4H), 4.61 (d, J=9.6 Hz, 1H), 4.49-4.34 (m, 3H), 4.30-4.21 (m, 1H), 4.09-3.99 (m, 2H), 3.75-3.58 (m, 4H), 3.06-2.94 (m, 2H), 2.48 (s, 3H), 2.13-2.03 (m, 1H), 1.95-1.85 (m, 1H), 0.95 (s, 9H). MS (ESI) m/z=532.0 [M+H]⁺

### Example 36: (2S,4R)-1-((S)-2-(3-(2-Aminoethoxy)propanamido)-3,3-dimethylbutanoyl)-4-hydroxy-N-(4-(4-methylthiazol-5-yl)benzyl)pyrrolidine-2-carboxamide (Linker 36)

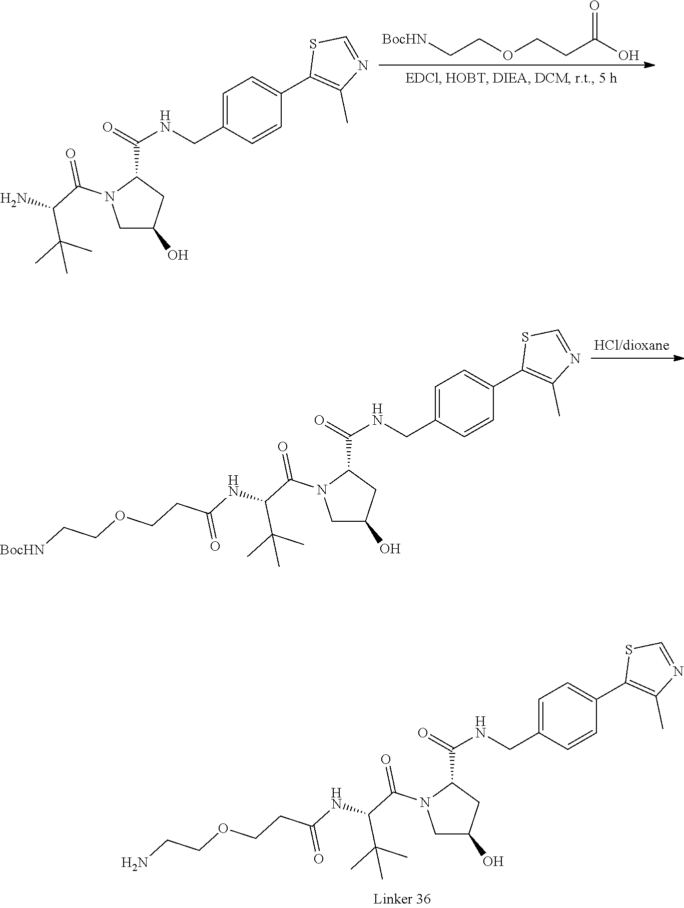 [SMILES: Cc1ncsc1-c1ccc(CNC(=O)[C@@H]2C[C@@H](O)CN2C(=O)[C@@H](N)C(C)(C)C)cc1 ; CC(C)(C)OC(=O)NCCOCCC(=O)O ; Cc1ncsc1-c1ccc(CNC(=O)[C@@H]2C[C@@H](O)CN2C(=O)[C@@H](NC(=O)CCOCCNC(=O)OC(C)(C)C)C(C)(C)C)cc1 ; Cc1ncsc1-c1ccc(CNC(=O)[C@@H]2C[C@@H](O)CN2C(=O)[C@@H](NC(=O)CCOCCN)C(C)(C)C)cc1]

Linker 36 was synthesized following the same procedures as Linker 25 as described in Example 25. (1.32 g, yield: 49% over 2 steps). ¹H NMR (400 MHz, DMSO-d₆) 8.99 (s, 1H), 8.57 (t, J=6.0 Hz, 1H), 8.03 (d, J=8 Hz, 1H), 7.85 (s, 3H), 7.43-7.37 (m, 4H), 4.57 (d, J=9.2 Hz, 1H), 4.46-4.31 (m, 3H), 4.26-4.20 (m, 1H), 3.69-3.55 (m, 6H), 3.99-2.95 (m, 2H), 2.60-2.56 (m, 1H), 2.46-2.42 (m, 4H), 2.05-2.03 (m, 1H), 1.93-1.92 (m, 1H), 0.95 (s, 9H). MS (ESI) m/z=546.0 [M+H]⁺.

### Example 37: (2S,4R)-1-((S)-2-(2-(2-(2-Aminoethoxy)ethoxy)acetamido)-3,3-dimethylbutanoyl)-4-hydroxy-N-(4-(4-methylthiazol-5-yl)benzyl)pyrrolidine-2-carboxamide (Linker 37)

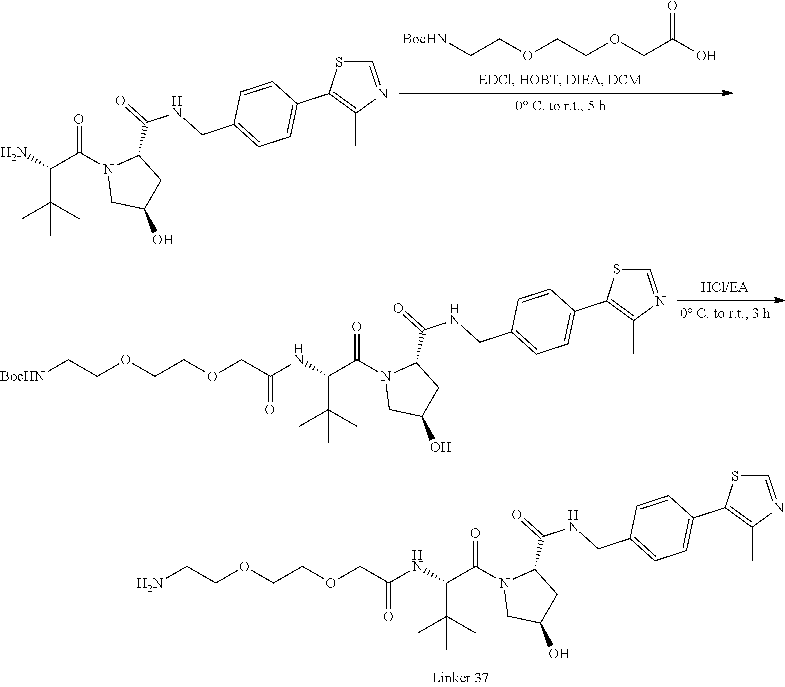 [SMILES: CC(C)(C)OC(=O)NCCOCCOCC(=O)O ; Cc1ncsc1-c1ccc(CNC(=O)[C@@H]2C[C@@H](O)CN2C(=O)[C@@H](N)C(C)(C)C)cc1 ; Cc1ncsc1-c1ccc(CNC(=O)[C@@H]2C[C@@H](O)CN2C(=O)[C@@H](NC(=O)COCCOCCNC(=O)OC(C)(C)C)C(C)(C)C)cc1 ; Cc1ncsc1-c1ccc(CNC(=O)[C@@H]2C[C@@H](O)CN2C(=O)[C@@H](NC(=O)COCCOCCN)C(C)(C)C)cc1]

Linker 37 was synthesized following the same procedures as Linker 25 as described in Example 25. (1.2 g, yield: 49% over 2 steps). ¹H NMR (400 MHz, DMSO-d6) δ 9.38 (s, 1H), 8.78 (t, J=6.0 Hz, 1H), 8.18 (s, 3H), 7.59-7.37 (m, 5H), 4.58 (d, J=9.6 Hz, 1H), 4.49 (t, J=8.2 Hz, 1H), 4.42-4.26 (m, 3H), 4.09-3.95 (m, 2H), 3.72-3.55 (m, 8H), 2.99-2.92 (m, 2H), 2.49 (s, 3H), 2.15-2.04 (m, 1H), 1.95-1.85 (m, 1H), 0.95 (s, 9H). MS (ESI) m/z=576.1 [M+H]⁺

### Example 38: (2S,4R)-14(S)-2-(3-(2-(2-Aminoethoxy)ethoxy)propanamido)-3,3-dimethylbutanoyl)-4-hydroxy-N-(4-(4-methylthiazol-5-yl)benzyl)pyrrolidine-2-carboxamide (Linker 38)

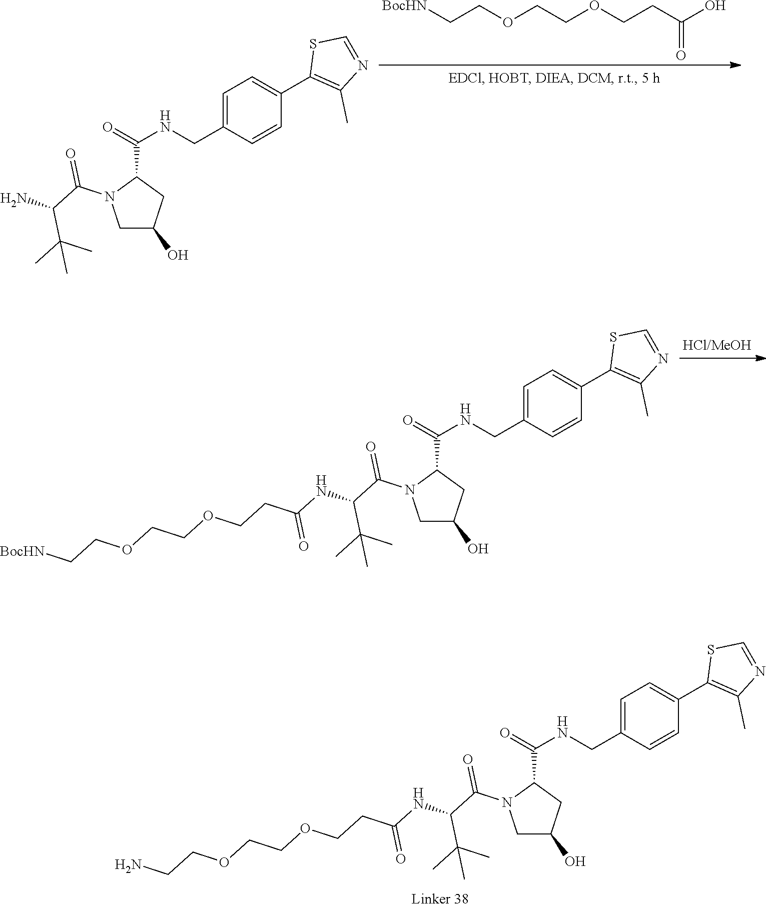 [SMILES: Cc1ncsc1-c1ccc(CNC(=O)[C@@H]2C[C@@H](O)CN2C(=O)[C@@H](N)C(C)(C)C)cc1 ; CC(C)(C)OC(=O)NCCOCCOCCC(=O)O ; Cc1ncsc1-c1ccc(CNC(=O)[C@@H]2C[C@@H](O)CN2C(=O)[C@@H](NC(=O)CCOCCOCCNC(=O)OC(C)(C)C)C(C)(C)C)cc1 ; Cc1ncsc1-c1ccc(CNC(=O)[C@@H]2C[C@@H](O)CN2C(=O)[C@@H](NC(=O)CCOCCOCCN)C(C)(C)C)cc1]

Linker 38 was synthesized following the same procedures as Linker 25 as described in Example 25. (1.34 g, yield: 49% over 2 steps). ¹H NMR (400 MHz, DMSO-d₆) 9.02 (s, 1H), 8.58 (t, J=6.0 Hz, 1H), 7.94 (d, J=8 Hz, 1H), 7.82 (s, 3H), 7.42-7.30 (m, 4H), 4.58 (d, J=9.2 Hz, 1H), 4.60-4.37 (m, 3H), 4.25-4.31 (m, 1H), 3.70-3.50 (m, 10H), 3.00-2.96 (m, 2H), 2.57-2.55 (m, 1H), 2.45 (s, 3H), 2.41-2.38 (m, 1H), 2.06-2.04 (m, 1H), 1.95-1.93 (m, 1H), 0.95 (s, 9H). MS (ESI) m/z=590.1 [M+H]⁺

### Example 39: (2S,4R)-1-((S)-14-Amino-2-(tert-butyl)-4-oxo-6,9,12-trioxa-3-azatetradecanoyl)-4-hydroxy-N-(4-(4-methylthiazol-5-yl)benzyl)pyrrolidine-2-carboxamide (Linker 39)

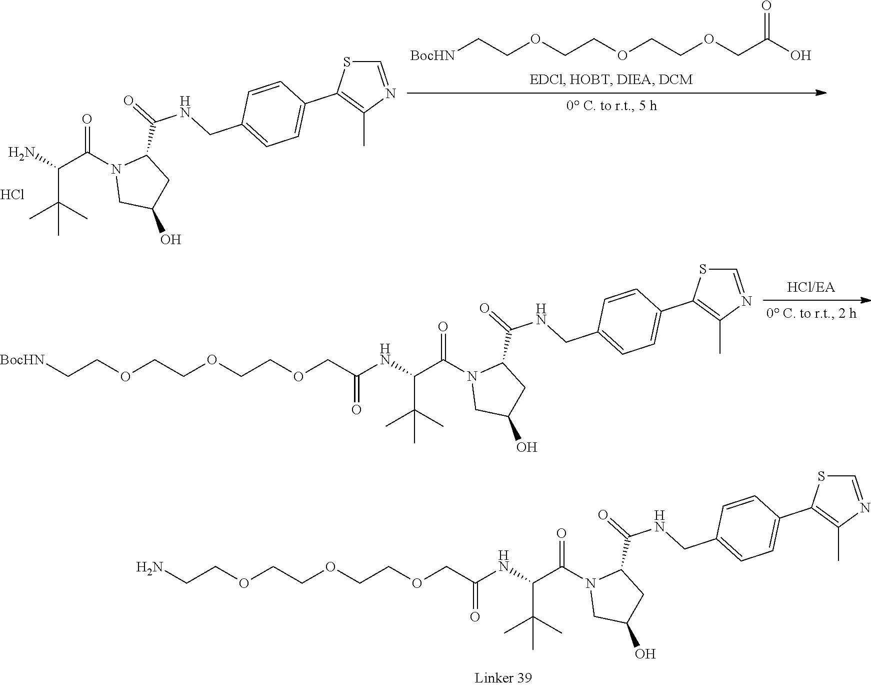 [SMILES: CC(C)(C)OC(=O)NCCOCCOCCOCC(=O)O ; Cl ; Cc1ncsc1-c1ccc(CNC(=O)[C@@H]2C[C@@H](O)CN2C(=O)[C@@H](N)C(C)(C)C)cc1 ; Cc1ncsc1-c1ccc(CNC(=O)[C@@H]2C[C@@H](O)CN2C(=O)[C@@H](NC(=O)COCCOCCOCCNC(=O)OC(C)(C)C)C(C)(C)C)cc1 ; Cc1ncsc1-c1ccc(CNC(=O)[C@@H]2C[C@@H](O)CN2C(=O)[C@@H](NC(=O)COCCOCCOCCN)C(C)(C)C)cc1]

Linker 39 was synthesized following the same procedures as Linker 25 as described in Example 25. (1.53 g, yield: 56% over 2 steps). ¹H NMR (400 MHz, DMSO-d6) δ 9.01 (s, 1H), 8.59 (t, J=6.0 Hz, 1H), 7.81 (s, 3H), 7.48-7.41 (m, 5H), 4.58 (d, J=9.6 Hz, 1H), 4.47-4.26 (m, 4H), 3.99 (s, 2H), 3.70-3.58 (m, 12H), 3.0-2.96 (m, 2H), 2.46 (s, 3H), 2.11-2.06 (m, 1H), 1.95-1.88 (m, 1H), 0.96 (s, 9H). MS (ESI) m/z=621.1 [M+H]⁺

### Example 40: (2S,4R)-1-((S)-1-Amino-14-(tert-butyl)-12-oxo-3,6,9-trioxa-13-azapentadecan-15-oyl)-4-hydroxy-N-(4-(4-methylthiazol-5-yl)benzyl)pyrrolidine-2-carboxamide (Linker 40)

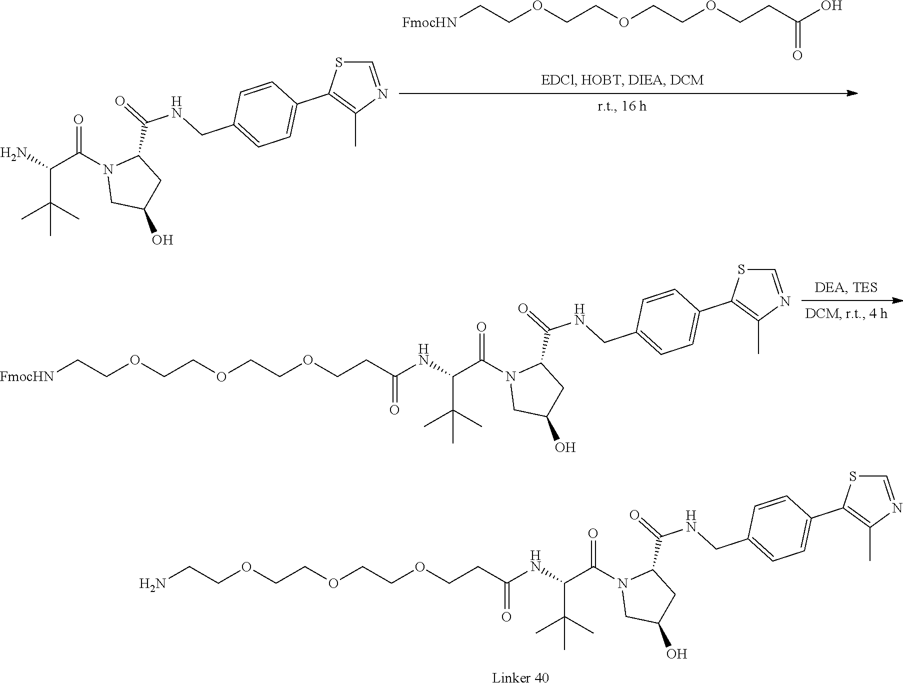

Linker 40 was synthesized following the same procedures as Linker 25 as described in Example 25. (1.52 g, yield: 51% over 2 steps). ¹H NMR (400 MHz, DMSO-d6) δ 9.01 (s, 1H), 8.57 (t, J=6.0 Hz, 1H), 7.91 (d, J=9.2 Hz, 1H), 7.81 (s, 3H), 7.44-7.38 (m, 4H), 4.58-4.55 (m, 1H), 4.45-4.36 (m, 3H), 4.25-4.21 (m, 1H), 3.70-3.48 (m, 14H), 3.00-2.97 (m, 2H), 2.59-2.52 (m, 1H), 2.46 (s, 3H), 2.39-2.34 (m, 1H), 2.08-2.03 (m, 1H), 1.95-1.88 (m, 1H), 0.94 (s, 9H). MS (ESI) m/z=633.8 [M+H]⁺

### Example 41: (2S,4R)-1-((S)-1-Amino-17-(tert-butyl)-15-oxo-3,6,9,12-tetraoxa-16-azaoctadecan-18-oyl)-4-hydroxy-N-(4-(4-methylthiazol-5-yl)benzyl)pyrrolidine-2-carboxamide (Linker 41)

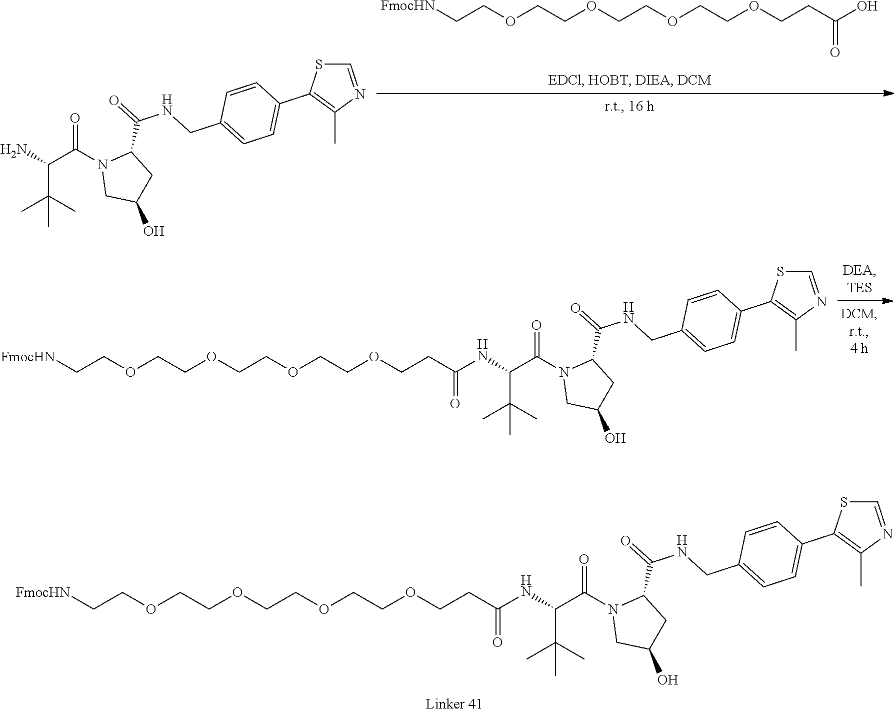

Linker 41 was synthesized following the same procedures as Linker 25 as described in Example 25. (1.12 g, yield: 37% over 2 steps). ¹H NMR (400 MHz, DMSO-d6) δ 8.98 (s, 1H), 8.58 (t, J=5.6 Hz, 1H), 7.92 (d, J=9.2 Hz, 1H), 7.44-7.38 (m, 4H), 4.56 (d, J=9.2 Hz, 1H), 4.47-4.41 (m, 2H), 4.38-4.34 (m, 1H), 4.26-4.19 (m, 1H), 3.70-3.55 (m, 5H), 3.53-3.45 (m, 14H), 3.35 (t, J=5.6 Hz, 2H), 2.64 (t, J=5.6 Hz, 2H), 2.58-2.50 (m, 1H), 2.45 (s, 3H), 2.40-2.35 (m, 1H), 2.08-2.00 (m, 1H), 1.94-1.91 (m, 1H), 0.94 (s, 9H). MS (ESI) m/z=678.1 [M+H]⁺

### Example 42: (2S,4R)-1-((S)-1-Amino-20-(tert-butyl)-18-oxo-3,6,9,12,15-pentaoxa-19-azahenicosan-21-oyl)-4-hydroxy-N-(4-(4-methylthiazol-5-yl)benzyl(pyrrolidine-2-carboxamide (Linker 42)

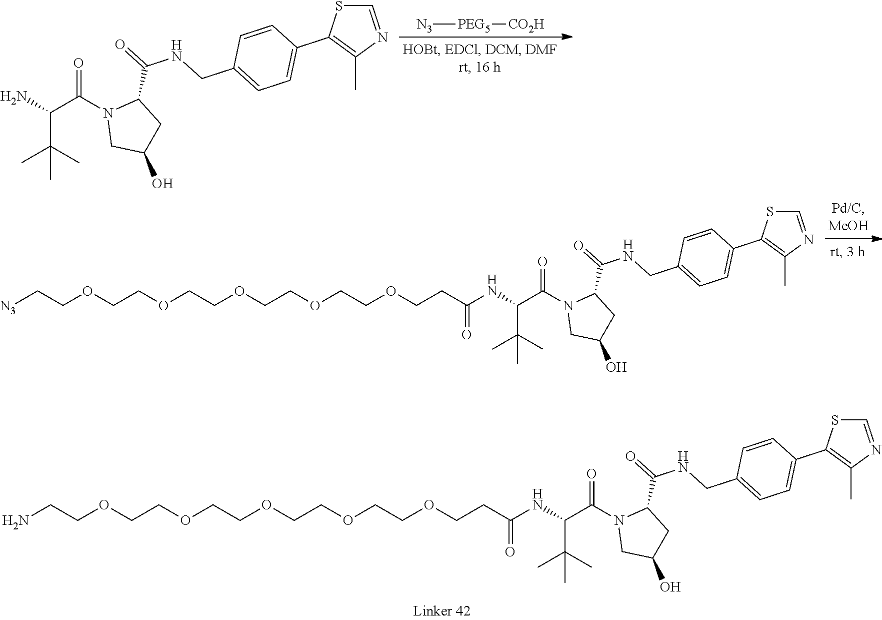

Linker 42 was synthesized following the same procedures as Linker 25 as described in Example 25. (1.1 g, 1.52 mmol, yield: 32% over 2 steps). ¹H NMR (400 MHz, DMSO-d₆) 9.38 (s, 1H), 8.67 (t, J=16 Hz, 1H), 8.14 (br, 3H), 7.91 (d, J=9.2 Hz, 1H), 7.39-7.48 (m, 4H), 4.53 (d, J=9.2 Hz, 1H), 4.39-4.46 (m, 2H), 4.36-4.34 (m, 1H), 4.20-4.25 (m, 1H), 3.45-3.68 (m, 22H), 2.91-2.95 (m, 2H), 2.52-2.58 (m, 1H), 2.47 (s, 3H), 2.32-2.39 (m, 1H), 2.03-2.08 (m, 1H), 1.85-1.92 (m, 1H), 0.92 (s, 9H). MS (ESI) m/z=722.4 [M+H]⁺

### Example 43: 4-(((S)-1-((2S,4R)-4-Hydroxy-24(4-(4-methylthiazol-5-yl)benzyl)carbamoyl)pyrrolidin-1-yl)-3,3-dimethyl-1-oxobutan-2-yl)amino)-4-oxobutanoic acid (Linker 43)

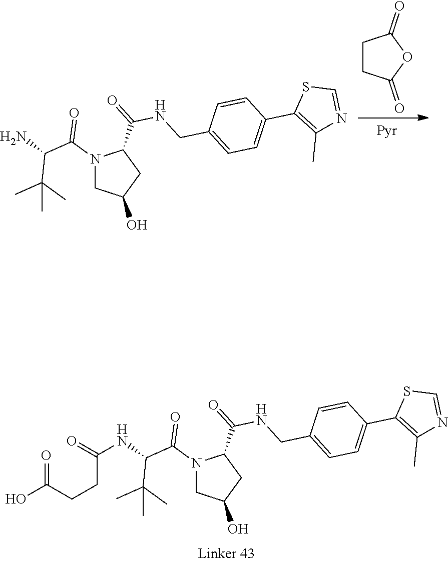

A mixture of (2S,4R)-1-((S)-2-amino-3,3-dimethylbutanoyl)-4-hydroxy-N-(4-(4-methylthiazol-5-yl)benzyl)pyrrolidine-2-carboxamide (1.0 g, 2.3 mmol) and succinic anhydride (465 mg, 4.65 mmol) in pyridine (5 mL) was stirred at rt for overnight. The mixture was concentrated. The residue was purified by flash chromatography (reversed-phase, MeCN/H₂O) to give the title compound Linker 43 (1.05 g, yield: 86%). ¹H NMR (400 MHz, DMSO-d₆): δ 12.02 (s, 1H), 8.99 (s, 1H), 8.58 (t, J=6.0 Hz, 1H), 7.96 (d, J=9.2 Hz, 1H), 7.43-7.37 (m, 4H), 5.13 (d, J=3.6 Hz, 1H), 4.53 (d, J=9.2 Hz, 1H), 4.46-4.40 (m, 2H), 4.34 (s, 1H), 4.21 (dd, J=16.0, 5.2 Hz, 1H), 3.69-3.60 (m, 2H), 2.45 (s, 3H), 2.44-2.33 (m, 4H), 2.06-2.01 (m, 1H), 1.93-1.87 (m, 1H), 0.93 (s, 9H). ¹³C NMR (100 MHz, DMSO-d6): δ173.83, 171.92, 170.86, 169.56, 151.41, 147.70, 139.48, 131.15, 129.63, 128.62, 127.41, 68.87, 58.70, 56.44, 56.34, 41.65, 37.91, 35.35, 29.74, 29.25, 26.35, 15.92. MS (ESI) m/z=531.2 [M+H]⁺

### Example 44: 5-(((S)-1-((2S,4R)-4-Hydroxy-2-((4-(4-methylthiazol-5-yl)benzyl)carbamoyl)pyrrolidin-1-yl)-3,3-dimethyl-1-oxobutan-2-yl)amino)-5-oxopentanoic acid (Linker 44)

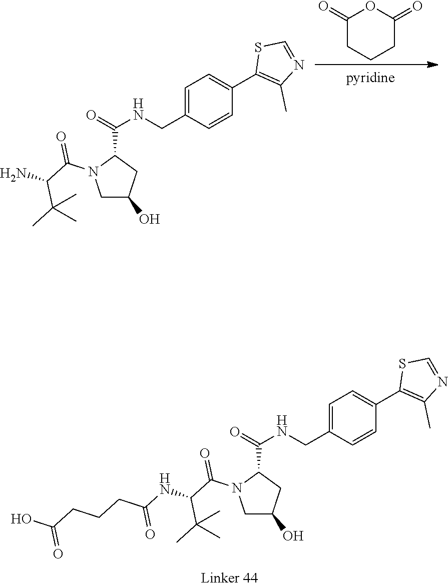 [SMILES: Cc1ncsc1-c1ccc(CNC(=O)[C@@H]2C[C@@H](O)CN2C(=O)[C@@H](N)C(C)(C)C)cc1 ; O=C1CCCC(=O)O1 ; Cc1ncsc1-c1ccc(CNC(=O)[C@@H]2C[C@@H](O)CN2C(=O)[C@@H](NC(=O)CCCC(=O)O)C(C)(C)C)cc1]

Linker 44 was synthesized following the same procedures as Linker 43 as described in Example 43. (1.5 g, yield: 79%). ¹H NMR (400 MHz, DMSO-d₆): δ 8.99 (s, 1H), 8.59 (t, J=6.0 Hz, 1H), 7.91 (d, J=9.2 Hz, 1H), 7.44-7.37 (m, 4H), 5.16 (brs, 1H), 4.54 (d, J=9.2 Hz, 1H), 4.47-4.42 (m, 2H), 4.36 (s, 1H), 4.21 (dd, J=16.0, 5.2 Hz, 1H), 3.7-3.64 (m, 2H), 2.45 (s, 3H), 2.31-2.14 (m, 4H), 2.07-2.02 (m, 1H), 1.94-1.81 (m, 1H), 1.74-1.68 (m, 2H), 0.94 (s, 9H). ¹³C NMR (100 MHz, DMSO-d₆): δ 174.18, 171.94, 171.63, 169.66, 151.41, 147.70, 139.46, 131.15, 129.61, 128.62, 127.41, 68.86, 58.69, 56.38, 41.65, 37.91, 35.16, 34.03, 33.10, 26.35, 20.89, 15.92. MS (ESI) m/z=543.2 [M−H]⁻

### Example 45: 6-(((S)-1-((2S,4R)-4-Hydroxy-2-((4-(4-methylthiazol-5-yl)benzyl)carbamoyl)pyrrolidin-1-yl)-3,3-dimethyl-1-oxobutan-2-yl)amino)-6-oxohexanoic acid (Linker 45)

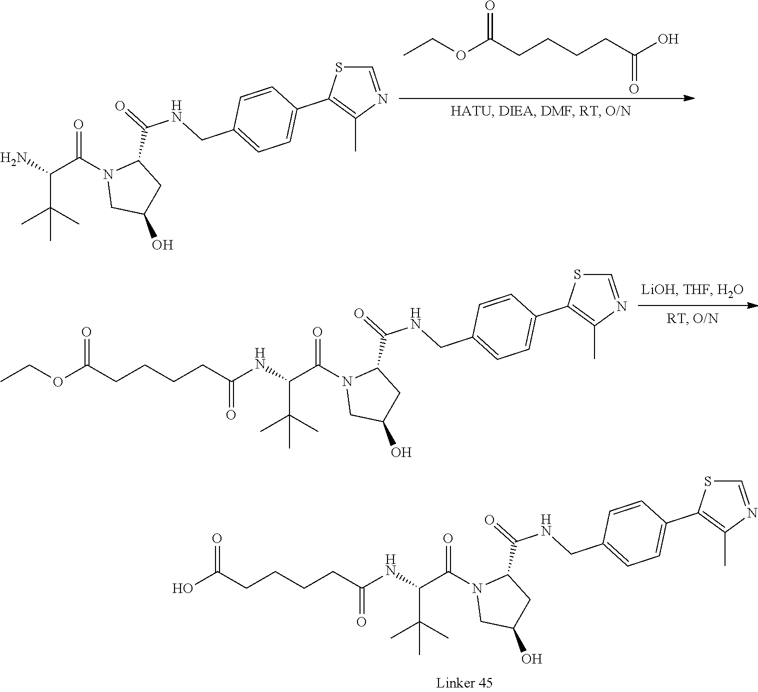

Linker 45 was synthesized following the same procedures as Linker 25 as described in Example 25. (1.2 g, yield: 55% over 2 steps). ¹H NMR (400 MHz, CDCl₃) 8.68 (s, 1H), 7.75 (s, 1H), 7.32-7.27 (m, 5H), 4.64-4.57 (m, 3H), 4.56-4.50 (m, 1H), 4.28-4.25 (m, 1H), 4.02-3.99 (m, 1H), 3.71-3.68 (m, 1H), 2.47 (s, 3H), 2.24-2.18 (m, 6H), 1.59-1.48 (m, 4H), 0.96 (s, 9H). MS (ESI) m/z=559.3 [M+H]⁺

### Example 46: 7-(((S)-1-((2S,4R)-4-Hydroxy-2-((4-(4-methylthiazol-5-yl)benzyl)carbamoyl)pyrrolidin-1-yl)-3,3-dimethyl-1-oxobutan-2-yl)amino)-7-oxoheptanoic acid (Linker 46)

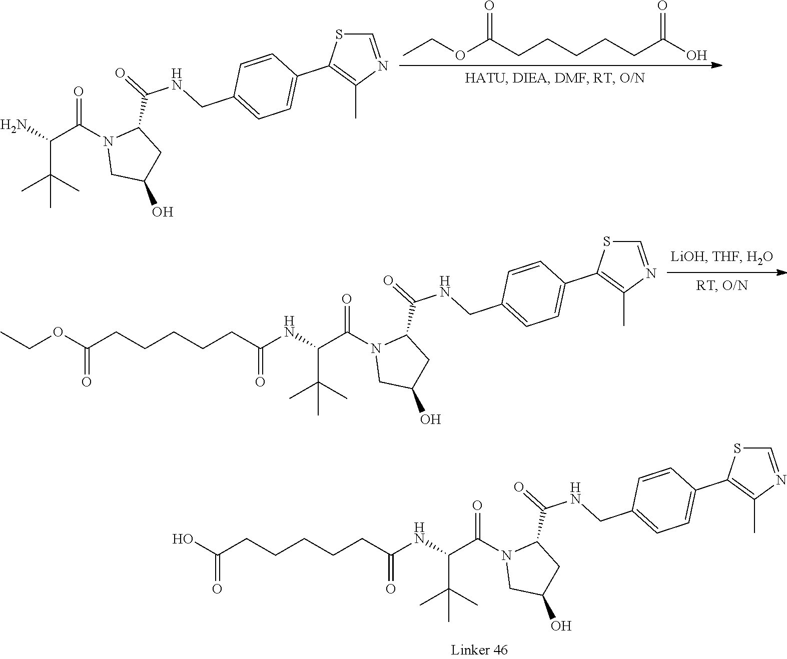

Linker 46 was synthesized following the same procedures as Linker 45 as described in Example 45. (1.1 g, yield: 33% over 2 steps). ¹H NMR (400 MHz, CDCl₃) 8.67 (s, 1H), 7.56-7.55 (m, 1H), 7.34-7.30 (m, 5H), 4.68-4.59 (m, 3H), 4.59-4.51 (m, 1H), 4.25 (dd, J=4.8 Hz, 15.2 Hz, 1H), 4.06-4.03 (m, 1H), 3.70-3.68 (m, 1H), 2.46 (s, 3H), 2.31-2.11 (m, 6H), 1.55-1.51 (m, 4H), 1.29-1.24 (m, 2H), 0.94 (s, 9H). MS (ESI) m/z=573.1 [M+H]⁺

### Example 47: 8-(((S)-1-((2S,4R)-4-Hydroxy-2-((4-(4-methylthiazol-5-yl)benzyl)carbamoyl)pyrrolidin-1-yl)-3,3-dimethyl-1-oxobutan-2-yl)amino)-8-oxooctanoic acid (Linker 47)

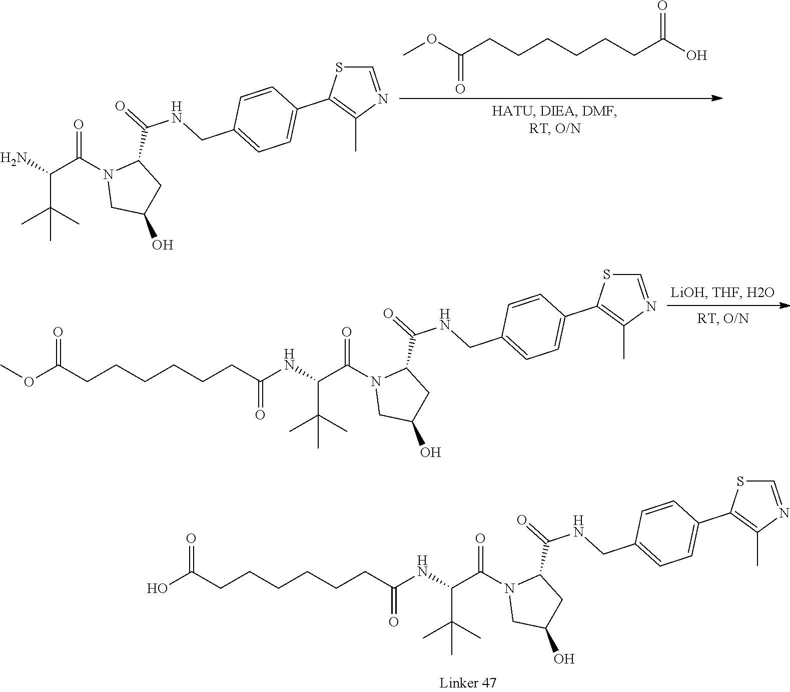

Linker 47 was synthesized following the same procedures as Linker 45 as described in Example 45. (1.08 g, yield: 52% over 2 steps). ¹H NMR (400 MHz, DMSO-d6) 8.99 (s, 1H), 8.55 (t, J=2.4 Hz, 1H), 7.83 (d, J=9.2 Hz, 1H), 7.44-7.38 (m, 4H), 4.55 (d, J=9.6 Hz, 1H), 4.52-4.41 (m, 2H), 4.36 (s, 1H), 4.25-4.21 (m, 1H), 3.67-3.66 (m, 2H), 2.45 (s, 3H), 2.30-1.91 (m, 6H), 1.49-1.47 (m, 4H), 1.26-1.24 (m, 4H), 0.92 (s, 9H). MS (ESI) m/z=587.3 [M+H]⁺

### Example 48: 9-(((S)-1-((2S,4R)-4-Hydroxy-2-((4-(4-methylthiazol-5-yl)benzyl)carbamoyl)pyrrolidin-1-yl)-3,3-dimethyl-1-oxobutan-2-yl)amino)-9-oxononanoic acid (Linker 48)

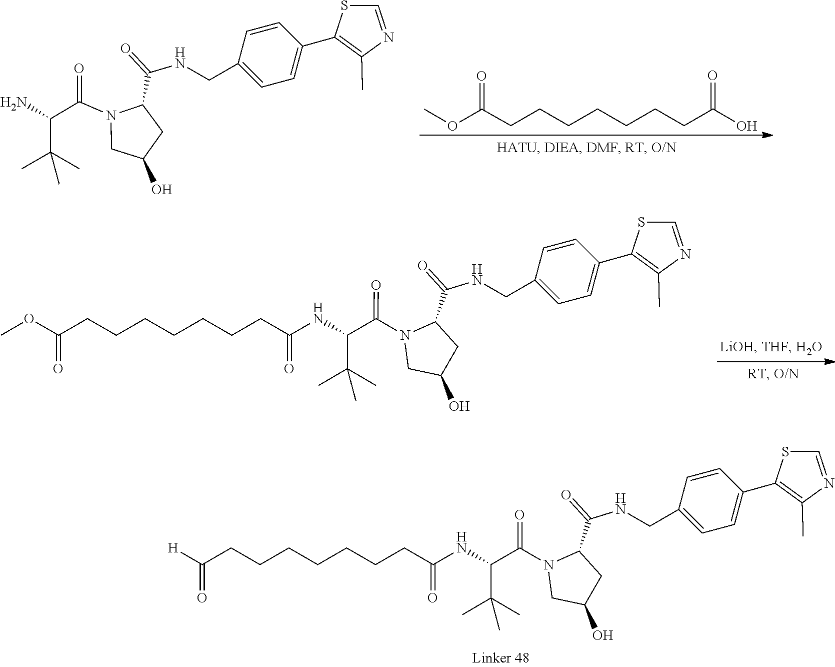 [SMILES: Cc1ncsc1-c1ccc(CNC(=O)[C@@H]2C[C@@H](O)CN2C(=O)[C@@H](N)C(C)(C)C)cc1 ; COC(=O)CCCCCCCC(=O)O ; COC(=O)CCCCCCCC(=O)N[C@H](C(=O)N1C[C@H](O)C[C@H]1C(=O)NCc1ccc(-c2scnc2C)cc1)C(C)(C)C ; Cc1ncsc1-c1ccc(CNC(=O)[C@@H]2C[C@@H](O)CN2C(=O)[C@@H](NC(=O)CCCCCCCC=O)C(C)(C)C)cc1]

Linker 48 was synthesized following the same procedures as Linker 45 as described in Example 45. (1.16 g, yield: 44% over 2 steps). ¹H NMR (400 MHz, CDCl₃) 8.70 (s, 1H), 7.55 (s, 1H), 7.33-7.27 (m, 4H), 7.08 (d, J=8.0 Hz, 1H), 4.68-4.52 (m, 4H), 4.31-4.27 (m, 1H), 4.08-4.05 (m, 1H), 3.69-3.67 (m, 1H), 2.48 (s, 3H), 2.33-2.11 (m, 6H), 1.60-1.47 (m, 4H), 1.29-1.20 (m, 6H), 0.96 (s, 9H). MS (ESI) m/z=601.1 [M+H]⁺

### Example 49: 10-(((S)-1-(((2S,4R)-4-Hydroxy-2-((4-(4-methylthiazol-5-yl)benzyl)carbamoyl)pyrrolidin-1-yl)-3,3-dimethyl-1-oxobutan-2-yl)amino)-10-oxodecanoic acid (Linker 49)

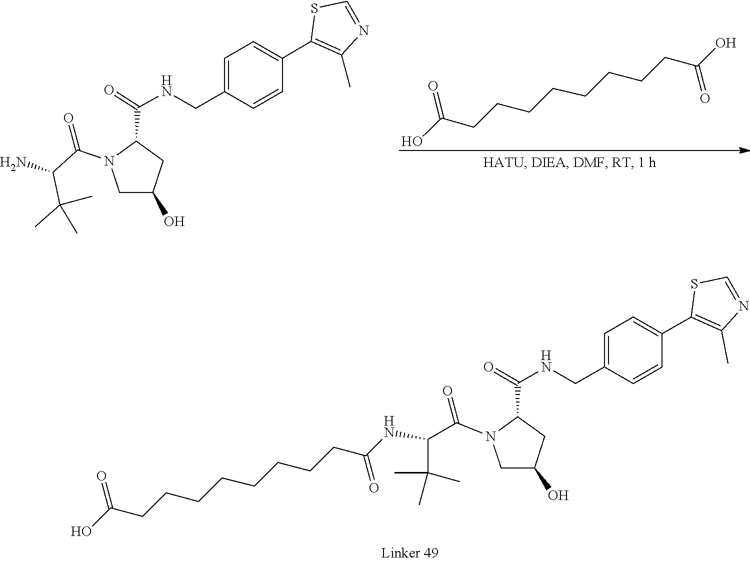 [SMILES: Cc1ncsc1-c1ccc(CNC(=O)[C@@H]2C[C@@H](O)CN2C(=O)[C@@H](N)C(C)(C)C)cc1 ; O=C(O)CCCCCCCCC(=O)O ; Cc1ncsc1-c1ccc(CNC(=O)[C@@H]2C[C@@H](O)CN2C(=O)[C@@H](NC(=O)CCCCCCCCC(=O)O)C(C)(C)C)cc1]

Linker 49 was synthesized following the same procedure as Linker 45 as described in Example 45. (1.1 g, yield: 35%). ¹H NMR (400 MHz, DMSO-d₆): δ8.99 (s, 1H), 8.58 (t, J=6.0 Hz, 1H), 7.85 (d, J=9.2 Hz, 1H), 7.43-7.37 (m, 4H), 4.54 (d, J=9.2 Hz, 1H), 4.47-4.41 (m, 2H), 4.35 (s, 1H), 4.21 (dd, J=16.0, 5.6 Hz, 1H), 3.69-3.63 (m, 2H), 2.45 (s, 3H), 2.29-2.09 (m, 4H), 2.03-2.01 (m, 1H), 1.94-1.88 (m, 1H), 1.47 (m, 4H), 1.24 (b, 8H), 0.94 (s, 9H). ¹³C NMR (100 MHz, DMSO-d6): δ 172.07, 171.92, 169.69, 151.41, 147.70, 139.48, 131.14, 129.62, 128.61, 127.40, 68.84, 58.67, 56.32, 56.26, 41.64, 37.93, 35.18, 34.85, 28.62, 26.36, 25.39, 15.93. MS (ESI) m/z=615.3 [M+H]⁺

### Example 50: 11-(((S)-1-((2S,4R)-4-Hydroxy-2-((4-(4-methylthiazol-5-yl)benzyl)carbamoyl)pyrrolidin-1-yl)-3,3-dimethyl-1-oxobutan-2-yl)amino)-11-oxoundecanoic acid (Linker 50)

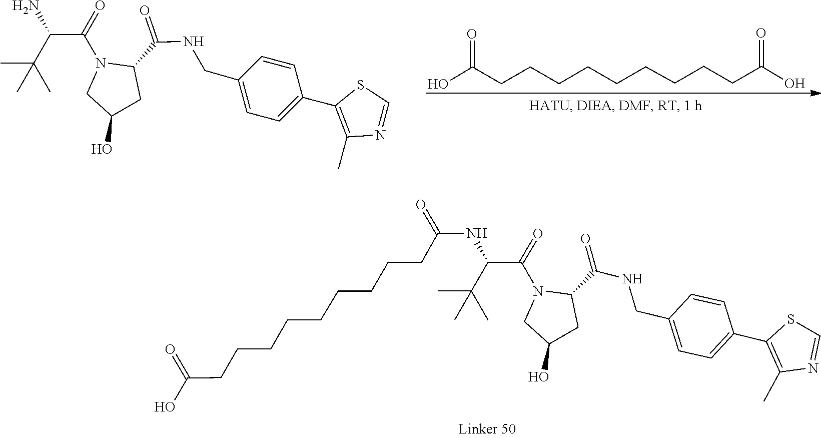 [SMILES: Cc1ncsc1-c1ccc(CNC(=O)[C@@H]2C[C@@H](O)CN2C(=O)[C@@H](N)C(C)(C)C)cc1 ; O=C(O)CCCCCCCCCC(=O)O ; Cc1ncsc1-c1ccc(CNC(=O)[C@@H]2C[C@@H](O)CN2C(=O)[C@@H](NC(=O)CCCCCCCCCC(=O)O)C(C)(C)C)cc1]

Linker 50 was synthesized following the same procedure as Linker 45 as described in Example 45. (1.1 g, yield: 50%). ¹H NMR (400 MHz, DMSO-d₆): δ8.99 (s, 1H), 8.58 (t, J=6.0 Hz, 1H), 7.85 (t, J=9.2 Hz, 1H), 7.37-7.43 (m, 4H), 4.56-4.19 (m, 5H), 3.70-3.60 (m, 2H), 2.45 (s, 3H), 2.27-1.90 (m, 6H), 1.49-1.45 (m, 4H), 1.23 (m, 10H), 0.93 (s, 9H). ¹³C NMR (100 MHz, DMSO-d6): δ174.59, 172.07, 171.92, 169.69, 151.42, 147.70, 139.49, 131.14, 129.62, 128.61, 127.41, 68.84, 58.67, 56.32, 56.25, 41.64, 37.93, 35.19, 34.85, 33.80, 28.82, 28.70, 28.68, 28.62, 28.55, 26.37, 25.42, 24.55, 15.93. MS (ESI) m/z=629.4 [M+H]⁺

### Example 51: 3-(3-(((S)-1-((2S,4R)-4-Hydroxy-2-((4-(4-methylthiazol-5-yl)benzyl)carbamoyl)pyrrolidin-1-yl)-3,3-dimethyl-1-oxobutan-2-yl)amino)-3-oxopropoxy)propanoic acid (Linker 51)

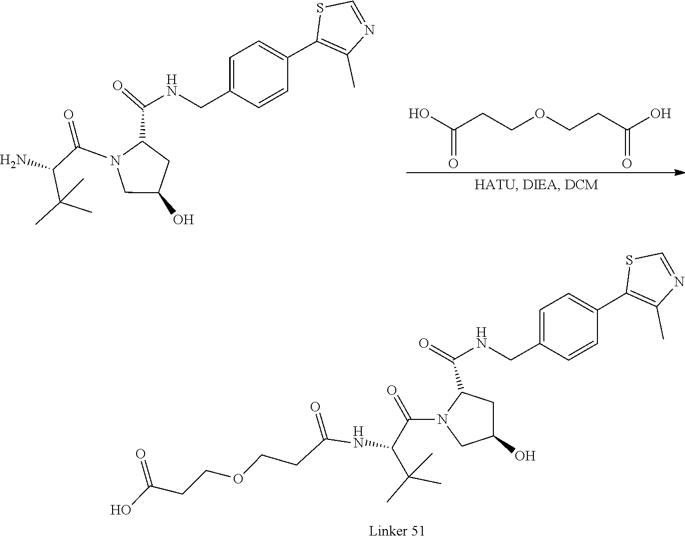

Linker 51 was synthesized following the same procedure as Linker 45 as described in Example 45. (1.1 g, yield: 42%). ¹H NMR (400 MHz, DMSO-d6) 8.98 (s, 1H), 8.55 (t, J=6.0 Hz, 1H), 7.91 (d, J=9.2 Hz, 1H), 7.43-7.37 (m, 4H), 4.55-4.53 (m, 1H), 4.45-4.40 (m, 2H), 4.35 (s, 1H), 4.24-4.19 (m, 1H), 3.68-3.52 (m, 6H), 2.54-2.56 (m, 1H), 2.45-2.37 (m, 5H), 2.34-2.30 (m, 1H), 2.05-2.00 (m, 1H), 1.93-1.86 (m, 1H), 0.93 (s, 9H). MS (ESI) m/z=575 [M+H]⁺

### Example 52: 2-(2-(((S)-1-((2S,4R)-4-Hydroxy-2-((4-(4-methylthiazol-5-yl)benzyl)carbamoyl)pyrrolidin-1-yl)-3,3-dimethyl-1-oxobutan-2-yl)amino)-2-oxoethoxy)acetic acid (Linker 52)

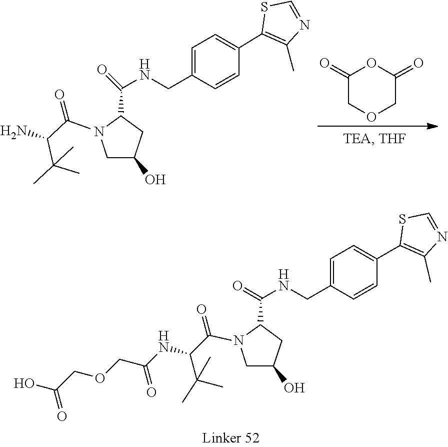

Linker 52 was synthesized following the same procedure as Linker 43 as described in Example 43. (1.2 g, yield: 63%). ¹H NMR (400 MHz, DMSO-d6) 12.81 (br s, 1H), 8.98 (s, 1H), 8.58 (t, J=6.0 Hz, 1H), 7.60 (d, J=9.6 Hz, 1H), 7.45-7.35 (m, 4H), 5.14 (br, 1H), 4.58-4.55 (m, 1H), 4.46-4.36 (m, 3H), 4.28-4.26 (m, 1H), 4.14 (s, 2H), 4.04 (s, 2H), 3.69-3.60 (m, 2H), 2.44 (s, 3H), 2.08-2.03 (m, 1H), 1.93-1.87 (m, 1H), 0.95 (s, 9H). MS (ESI) m/z=547 [M+H]⁺

### Example 53: 3-(2-(3-(((S)-1-((2S,4R)-4-Hydroxy-2-((4-(4-methylthiazol-5-yl)benzyl)carbamoyl)pyrrolidin-1-yl)-3,3-dimethyl-1-oxobutan-2-yl)amino)-3-oxopropoxy)ethoxy)propanoic acid (Linker 53)

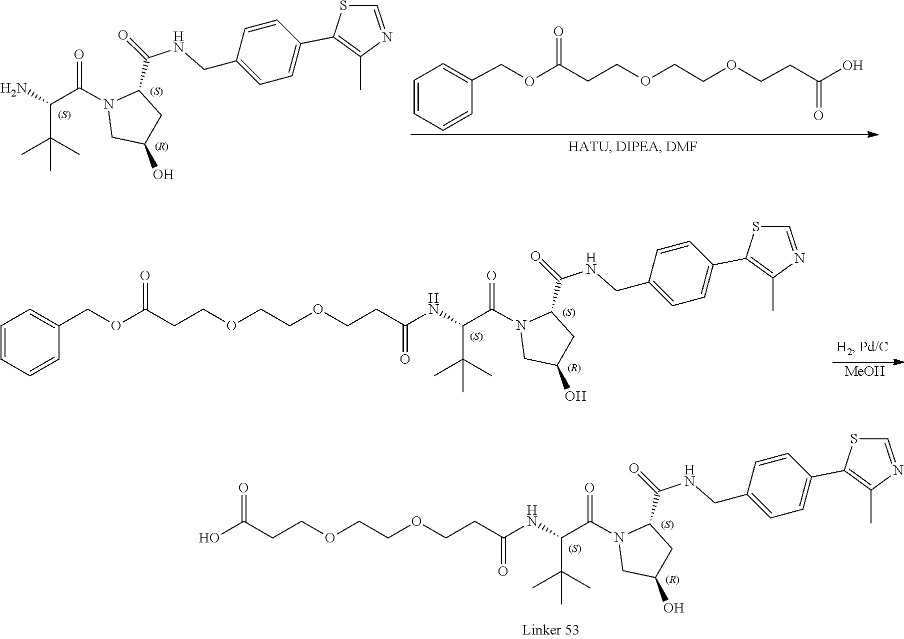

Linker 53 was synthesized following the same procedures as Linker 45 as described in Example 45. (1.4 g, yield 23% over 2 steps). ¹H NMR (400 MHz, DMSO-d₆): 8.98 (s, 1H), 8.56 (t, J=6.0 Hz, 1H), 7.91 (d, J=9.2 Hz, 1H), 7.43-7.37 (m, 4H), 4.55 (d, J=9.6 Hz, 1H), 4.46-4.41 (m, 2H), 4.35 (s, 1H), 4.29-4.20 (m, 1H), 3.70-3.57 (m, 7H), 3.50-3.45 (m, 5H), 2.57-2.55 (m, 1H), 2.45 (s, 3H), 2.43-2.41 (m, 1H), 2.37-2.32 (m, 1H), 2.09-2.01 (m, 1H), 1.94-1.87 (m, 1H), 0.94 (s, 9H). MS (ESI) m/z=619.3 [M+H]⁺

### Example 54: 2-(2-(2-(((S)-1-((2S,4R)-4-Hydroxy-2-((4-(4-methylthiazol-5-yl)benzyl)carbamoyl)pyrrolidin-1-yl)-3,3-dimethyl-1-oxobutan-2-yl)amino)-2-oxoethoxy)ethoxy)acetic acid (Linker 54)

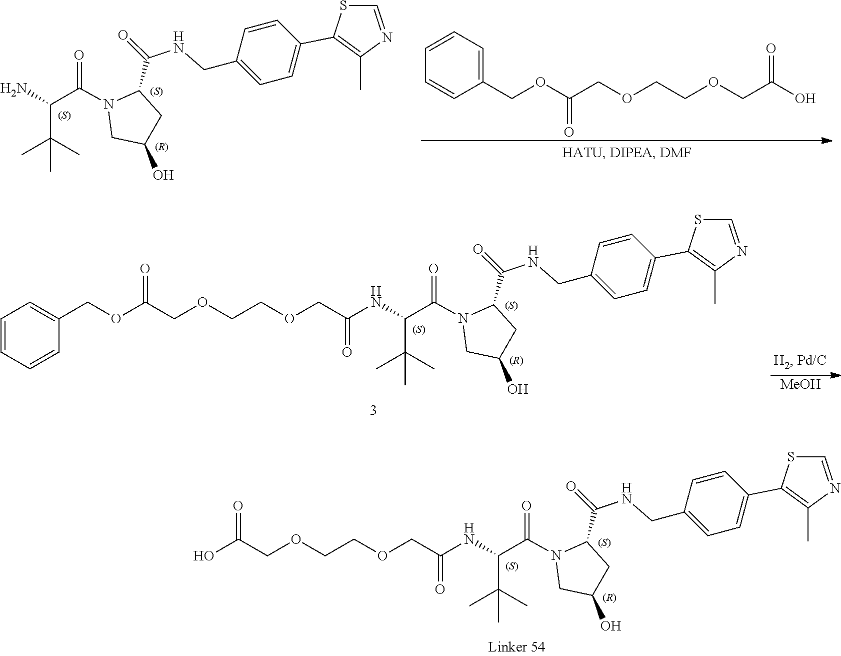

Linker 54 was synthesized following the same procedures as Linker 53 as described in Example 53. (1.13 g, yield 20% over 2 steps). ¹H NMR (400 MHz, DMSO-d₆): 8.98 (s, 1H), 8.60 (t, J=6.0 Hz, 1H), 7.49 (d, J=9.2 Hz, 1H), 7.40 (s, 4H), 4.57 (d, J=9.2 Hz, 1H), 4.47-4.36 (m, 3H), 4.28-4.23 (m, 1H), 4.05-3.93 (m, 4H), 3.69-3.61 (m, 6H), 2.45 (s, 3H), 2.08-2.03 (m, 1H), 1.94-1.87 (m, 1H), 0.94 (s, 9H). MS (ESI) m/z=591.2 [M+H]⁺

### Example 55: (S)-15-((2S,4R)-4-Hydroxy-2-((4-(4-methylthiazol-5-yl)benzyl)carbamoyl)pyrrolidine-1-carbonyl)-16,16-dimethyl-13-oxo-4,7,10-trioxa-14-azaheptadecanoic acid (Linker 55)

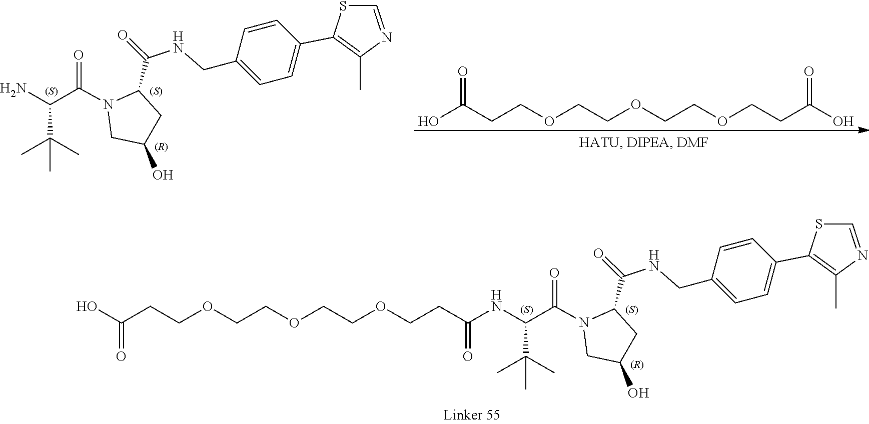

Linker 55 was synthesized following the same procedure as Linker 45 as described in Example 45. (1.7 g, yield 37%). ¹H NMR (400 MHz, DMSO-d₆): 8.99 (s, 1H), 8.56 (t, J=6.0 Hz, 1H), 7.91 (d, J=9.6 Hz, 1H), 7.44-7.38 (m, 4H), 4.56 (d, J=9.2 Hz, 1H), 4.47-4.42 (m, 2H), 4.36 (s, 1H), 4.25-4.20 (m, 1H), 3.70-3.55 (m, 6H), 3.50-3.46 (m, 8H), 2.58-2.51 (m, 3H), 2.45-2.42 (m, 5H), 2.40-2.33 (m, 1H), 2.07-2.02 (m, 1H), 1.94-1.88 (m, 1H), 0.94 (s, 9H). LCMS (ESI) m/z=661.0 [M−H]⁻

### Example 56: (S)-13-((2S,4R)-4-Hydroxy-2-((4-(4-methylthiazol-5-yl)benzyl)carbamoyl)pyrrolidine-1-carbonyl)-14,14-dimethyl-11-oxo-3,6,9-trioxa-12-azapentadecanoic acid (Linker 56)

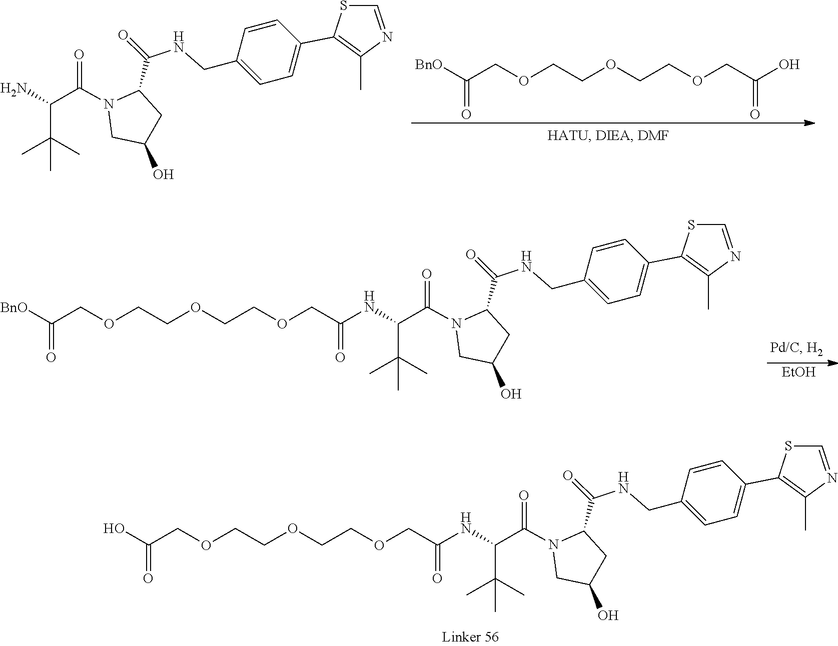 [SMILES: Cc1ncsc1-c1ccc(CNC(=O)[C@@H]2C[C@@H](O)CN2C(=O)[C@@H](N)C(C)(C)C)cc1 ; O=C(O)COCCOCCOCC(=O)OCc1ccccc1 ; "EtOH" ; Cc1ncsc1-c1ccc(CNC(=O)[C@@H]2C[C@@H](O)CN2C(=O)[C@@H](NC(=O)COCCOCCOCC(=O)OCc2ccccc2)C(C)(C)C)cc1 ; Cc1ncsc1-c1ccc(CNC(=O)[C@@H]2C[C@@H](O)CN2C(=O)[C@@H](NC(=O)COCCOCCOCC(=O)O)C(C)(C)C)cc1]

Linker 56 was synthesized following the same procedures as Linker 45 as described in Example 45. (1.21 g, yield 31% over 2 steps). ¹H NMR (400 MHz, CDCl₃): δ 8.68 (s, 1H), 7.80-7.71 (m, 11H), 7.41-7.33 (m, 5H), 4.71-7.65 (m, 1H), 4.61-4.50 (m, 3H), 4.37-4.33 (m, 1H), 4.07-3.94 (m, 5H), 3.77-3.58 (m, 10H), 2.51 (s, 3H), 2.38-2.30 (m, 1H), 2.24-2.19 (m, 1H), 0.98 (s, 9H). LCMS (ESI) m/z=635.0 [M+H]⁺

### Example 57: (S)-18-((2S,4R)-4-Hydroxy-2-((4-(4-methylthiazol-5-yl)benzyl)carbamoyl)pyrrolidine-1-carbonyl)-19,19-dimethyl-16-oxo-4,7,10,13-tetraoxa-17-azaicosanoic acid (Linker 57)

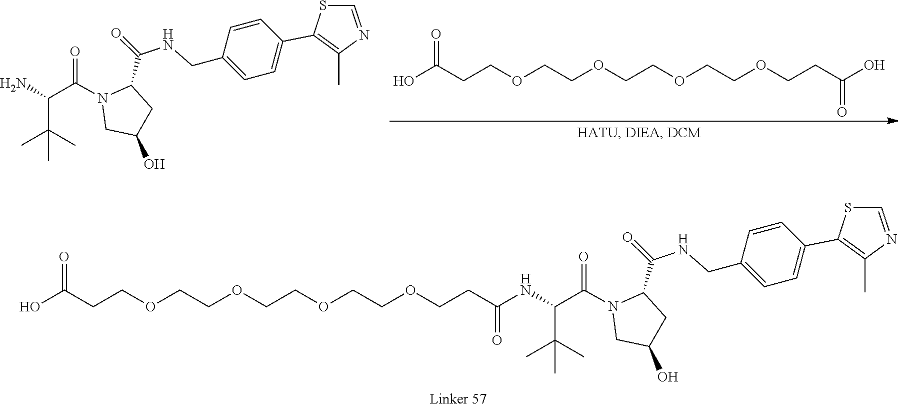 [SMILES: Cc1ncsc1-c1ccc(CNC(=O)[C@@H]2C[C@@H](O)CN2C(=O)[C@@H](N)C(C)(C)C)cc1 ; O=C(O)CCOCCOCCOCCOCCC(=O)O ; Cc1ncsc1-c1ccc(CNC(=O)[C@@H]2C[C@@H](O)CN2C(=O)[C@@H](NC(=O)CCOCCOCCOCCOCCC(=O)O)C(C)(C)C)cc1]

Linker 57 was synthesized following the same procedure as Linker 45 as described in Example 45. (1.6 g, yield 43%). ¹H NMR (400 MHz, CDCl₃): δ 8.69 (s, 1H), 7.55-7.52 (m, 1H), 7.47-7.45 (m, 1H), 7.36 (s, 4H), 4.70-4.66 (m, 1H), 4.62-4.57 (m, 2H), 4.50 (s, 1H), 4.34-4.29 (m, 1H), 4.12-4.09 (m, 1H), 3.75-3.48 (m, 18H), 2.56-2.47 (m, 7H), 2.40-2.33 (m, 1H), 2.23-2.18 (m, 1H), 0.96 (s, 9H). MS (ESI) m/z=707.1 [M+H]⁺

### Example 58: (S)-21-((2S,4R)-4-Hydroxy-2-((4-(4-methylthiazol-5-yl)benzyl)carbamoyl)pyrrolidine-1-carbonyl)-22,22-dimethyl-19-oxo-4,7,10,13,16-pentaoxa-20-azatricosanoic acid (Linker 58)

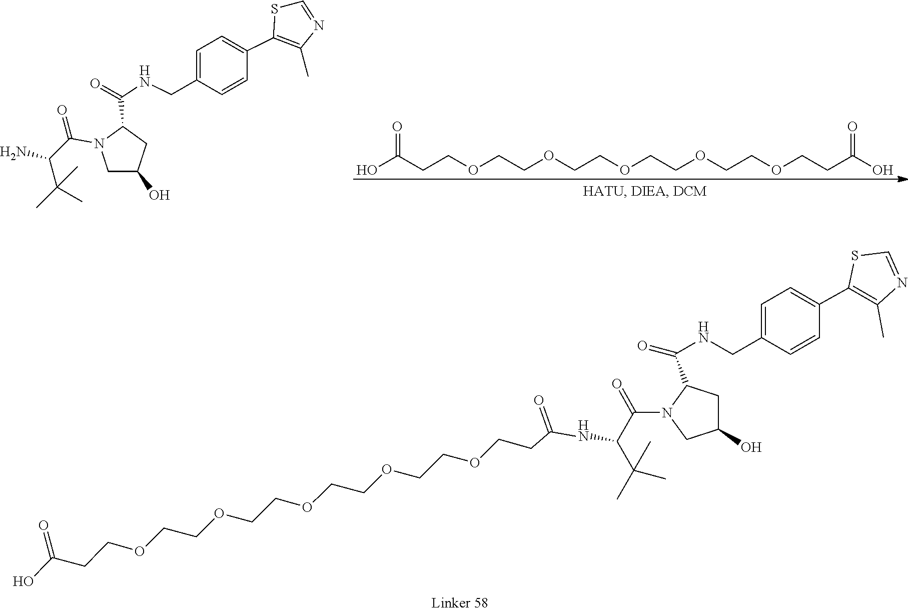

Linker 58 was synthesized following the same procedure as Linker 45 as described in Example 45. (1.2 g, yield: 23%). ¹H NMR (400 MHz, DMSO-d6) 8.98 (s, 1H), 8.57 (t, J=6.0 Hz, 1H), 7.91 (d, J=9.6 Hz, 1H), 7.43-7.31 (m, 4H), 4.56-4.53 (m, 1H), 4.45-4.35 (m, 3H), 4.24-4.19 (m, 1H), 3.69-3.55 (m, 6H), 3.49-3.47 (m, 16H), 2.57-2.53 (m, 1H), 2.45 (s, 3H), 2.39-2.32 (m, 3H), 2.06-2.01 (m, 1H), 1.93-1.86 (m, 1H), 0.95 (s, 9H). MS (ESI) m/z=751 [M+H]⁺

### Example 59: (S)-19-((2S,4R)-4-Hydroxy-2-((4-(4-methylthiazol-5-yl)benzyl)carbamoyl)pyrrolidine-1-carbonyl)-20,20-dimethyl-17-oxo-3,6,9,12,15-pentaoxa-18-azahenicosanoic acid (Linker 59)

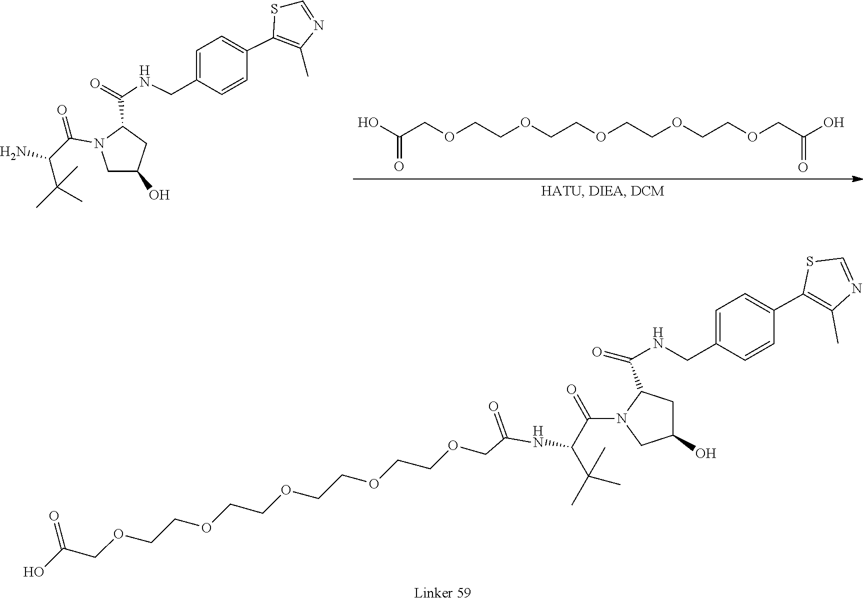

Linker 59 was synthesized following the same procedure as Linker 45 as described in Example 45. (1.3 g, yield: 39%). ¹H NMR (400 MHz, DMSO-d6) 8.98 (s, 1H), 8.69 (t, J=6.0 Hz, 1H), 7.45 (d, J=9.6 Hz, 1H), 7.43-7.37 (m, 4H), 4.57-4.55 (m, 1H), 4.47-4.34 (m, 3H), 4.27-4.22 (m, 1H), 3.97 (s, 2H), 3.68-3.65 (m, 2H), 3.61-3.48 (m, 18H), 2.45 (s, 3H), 2.09-2.04 (m, 1H), 1.92-1.86 (m, 1H), 0.94 (s, 9H). MS (ESI) m/z=723 [M+H]⁺

### Example 60: 5-((2-(2-Aminoethoxy)ethyl)amino)-2-(2,6-dioxopiperidin-3-yl)isoindoline-1,3-dione (Linker 60)

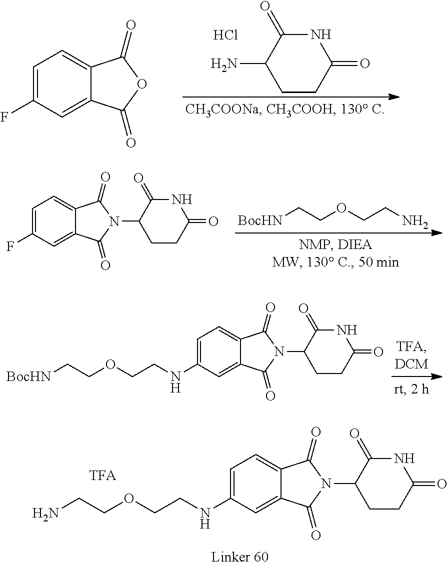

A mixture of 5-fluoroisobenzofuran-1,3-dione (87 g, 524 mmol), 3-aminopiperidine-2,6-dione (85.7 g, 524 mmol) and CH₃COONa (85.9 g, 1050 mmol) in CH₃COOH (500 mL) was stirred at 130° C. overnight. After cooling down to room temperature, the mixture was concentrated. The residue was poured into ice water, and filtered. The filter cake was washed with water (500 mL×2), EtOH (500 mL×2), MeOH (500 mL) and DCM (500 mL) to afford a solid which was dried in vacuum to give 2-(2,6-dioxopiperidin-3-yl)-5-fluoroisoindoline-1,3-dione (120 g, yield: 83%) as a yellow solid. MS (ESI) m/z=277.1 [M+H]⁺

A mixture of 2-(2,6-dioxopiperidin-3-yl)-5-fluoroisoindoline-1,3-dione (6.9 g, 25.0 mmol), tert-butyl (2-(2-aminoethoxy)ethyl)carbamate (5.6 g, 27.5 mmol) and DIEA (9.7 g, 75 mmol) in NMP (75 mL) was stirred at 130° C. in microwave reactor for 50 min. After cooling down to room temperature, the mixture was poured into EtOAc (200 mL), washed with water (200 mL×2) and brine (200 mL). The organic phase was dried over anhydrous Na₂SO₄, filtered and concentrated to give a crude product which was purified by chromatography on silica gel (petroleum ether/EtOAc=2:1 to 1:2) to give tert-butyl (2-(2-((2-(2,6-dioxopiperidin-3-yl)-1,3-dioxoisoindolin-5-yl)amino)ethoxy) ethyl)carbamate (2.4 g, yield: 21%) as a yellow oil. MS (ESI) m/z=361.1 [M+H]⁺

To a solution of tert-butyl (2-(2-((2-(2,6-dioxopiperidin-3-yl)-1,3-dioxoisoindolin-5-yl)amino)ethoxy)ethyl)carbamate (2.4 g, 5.2 mmol) in DCM (10 mL) was added TFA (5 mL) in one portion. The reaction mixture was stirred at room temperature for 2 hrs, and concentrated to dry. The residue was dissolved in water (20 mL), washed with EtOAc (40 mL) and methyl tertiary-butyl ether (MTBE) (40 mL). The aqueous phase was lyophilized to afford TFA salt of 5-((2-(2-aminoethoxy)ethyl)amino)-2-(2,6-dioxopiperidin-3-yl) isoindoline-1,3-dione (1.9 g, yield: 77%) as a yellow solid. MS (ESI) m/z=361.1 [M+H]⁺. ¹H NMR (400 MHz, DMSO-d₆) δ 11.06 (s, 1H), 8.01 (s, 3H), 7.58 (d, J=8.4 Hz, 1H), 7.12 (br, s, 1H), 7.02 (d, J=2.0 Hz, 1H), 6.91 (dd, J=2.0 Hz, 8.8 Hz, 1H), 5.04 (dd, J=5.6 Hz, 13.2 Hz, 1H), 3.64 (t, J=5.6 Hz, 4H), 3.40 (t, J=5.2 Hz, 2H), 3.01 (br, 2H), 2.89-2.83 (m, 1H), 2.60-2.50 (m, 2H), 2.03-1.97 (m, 1H).

### Example 61: 5-((2-(2-(2-Aminoethoxy)ethoxy)ethyl)amino)-2-(2,6-dioxopiperidin-3-yl)isoindoline-1,3-dione (Linker 61)

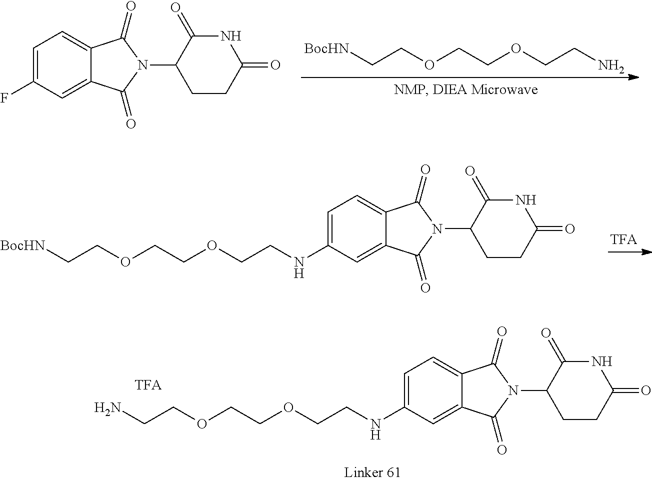 [SMILES: O=C1CCC(N2C(=O)c3ccc(F)cc3C2=O)C(=O)N1 ; CC(C)(C)OC(=O)NCCOCCOCCN ; CC(C)(C)OC(=O)NCCOCCOCCNc1ccc2c(c1)C(=O)N(C1CCC(=O)NC1=O)C2=O ; NCCOCCOCCNc1ccc2c(c1)C(=O)N(C1CCC(=O)NC1=O)C2=O]

Linker 61 was synthesized following the same procedure as Linker 60 as described in Example 60. (1.4 g, yield: 71%). MS (ESI) m/z=405.1 [M+H]⁺. ¹H NMR (400 MHz, DMSO-d₆) δ 11.05 (s, 1H), 7.94 (br, 3H), 7.56 (d, J=8.4 Hz, 1H), 7.01 (s, 1H), 6.90 (d, J=8.0 Hz, 1H), 5.03 (dd, J=5.2 Hz, 12.8 Hz, 1H), 3.58 (br, 8H), 3.36 (s, 2H), 2.97-2.92 (m, 2H), 2.91-2.83 (m, 1H), 2.60-2.50 (m, 2H), 2.01-1.99 (m, 1H).

### Example 62: 5-((2-(2-(2-(2-Aminoethoxy)ethoxy)ethoxy)ethyl)amino)-2-(2,6-dioxopiperidin-3-yl)isoindoline-1,3-dione (Linker 62)

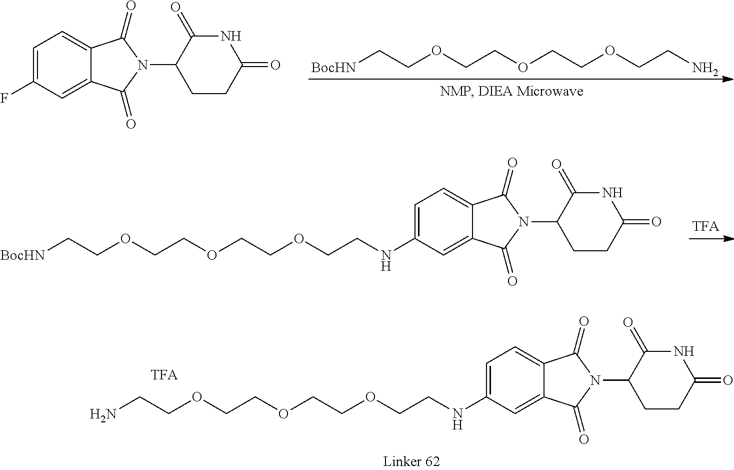 [SMILES: O=C1CCC(N2C(=O)c3ccc(F)cc3C2=O)C(=O)N1 ; CC(C)(C)OC(=O)NCCOCCOCCOCCN ; CC(C)(C)OC(=O)NCCOCCOCCOCCNc1ccc2c(c1)C(=O)N(C1CCC(=O)NC1=O)C2=O ; NCCOCCOCCOCCNc1ccc2c(c1)C(=O)N(C1CCC(=O)NC1=O)C2=O]

Linker 62 was synthesized following the same procedure as Linker 60 as described in Example 60. (1.19 g, yield: 59%). MS (ESI) m/z=449.1 [M+H]⁺. ¹H NMR (400 MHz, DMSO-d₆) δ 11.05 (s, 1H), 7.79 (br, 3H), 7.57 (d, J=8.4 Hz, 1H), 7.15 (br, s, 1H), 7.00 (d, J=2.0 Hz, 1H), 6.90 (dd, J=2.0 Hz, 8.4 Hz, 1H), 5.03 (dd, J=5.6 Hz, 12.8 Hz, 1H), 3.61-3.55 (m, 12H), 3.36 (t, J=5.6 Hz, 2H), 2.99-2.94 (m, 2H), 2.88-2.84 (m, 1H), 2.60-2.52 (m, 2H) 2.01-1.98 (m, 1H).

### Example 63: 5-((14-Amino-3,6,9,12-tetraoxatetradecyl)amino)-2-(2,6-dioxopiperidin-3-yl)isoindoline-1,3-dione (Linker 63)

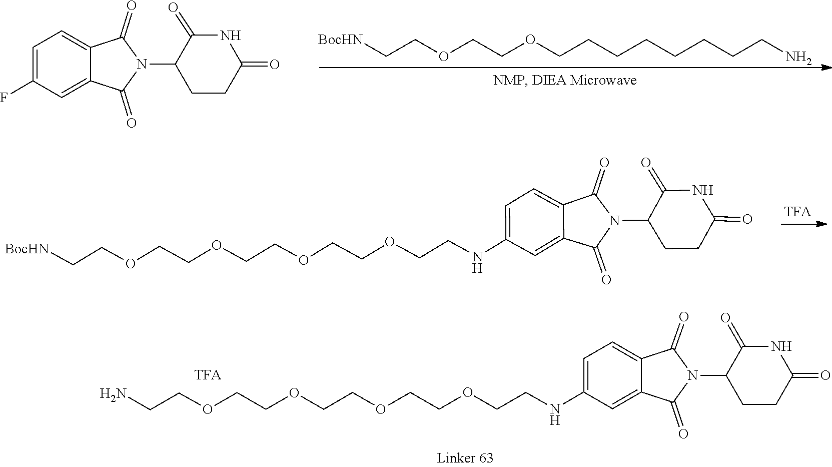 [SMILES: O=C1CCC(N2C(=O)c3ccc(F)cc3C2=O)C(=O)N1 ; CC(C)(C)OC(=O)NCCOCCOCCCCCCCCN ; CC(C)(C)OC(=O)NCCOCCOCCOCCOCCNc1ccc2c(c1)C(=O)N(C1CCC(=O)NC1=O)C2=O ; NCCOCCOCCOCCOCCNc1ccc2c(c1)C(=O)N(C1CCC(=O)NC1=O)C2=O]

Linker 63 was synthesized following the same procedure as Linker 60 as described in Example 60. (1.2 g, yield: 73%). MS (ESI) m/z=493.1 [M+H]⁺. ¹H NMR (400 MHz, DMSO-d6) δ 11.05 (s, 1H), 7.79 (br, J=1.6 Hz, 3H), 7.56 (d, J=8.4 Hz, 1H), 7.14 (br, s, 1H), 7.01 (d, J=2.0 Hz, 1H), 6.90 (dd, J=2.0 Hz, 8.4 Hz, 1H), 5.03 (dd, J=5.6 Hz, 13.2 Hz, 1H), 3.61-3.56 (m, 16H), 3.36 (t, J=5.2 Hz, 2H), 2.99-2.95 (m, 2H), 2.89-2.83 (m, 1H), 2.60-2.53 (m, 2H) 2.01-1.97 (m, 1H).

### Example 64: 5-((17-Amino-3,6,9,12,15-pentaoxaheptadecyl)amino)-2-(2,6-dioxopiperidin-3-yl)isoindoline-1,3-dione (Linker 64)

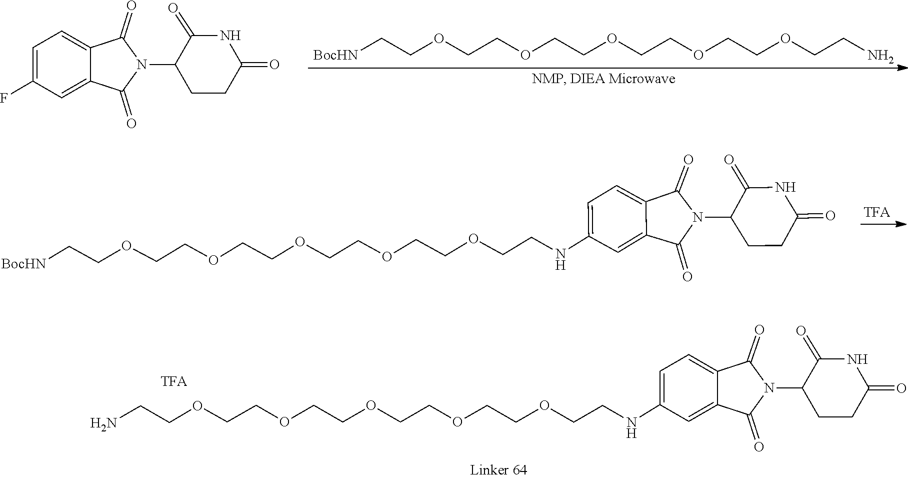 [SMILES: O=C1CCC(N2C(=O)c3ccc(F)cc3C2=O)C(=O)N1 ; CC(C)(C)OC(=O)NCCOCCOCCOCCOCCOCCN ; CC(C)(C)OC(=O)NCCOCCOCCOCCOCCOCCNc1ccc2c(c1)C(=O)N(C1CCC(=O)NC1=O)C2=O ; NCCOCCOCCOCCOCCOCCNc1ccc2c(c1)C(=O)N(C1CCC(=O)NC1=O)C2=O]

Linker 64 was synthesized following the same procedure as Linker 60 as described in Example 60. (1.73 g, yield: 88%). MS (ESI) m/z=537.2 [M+H]⁺. ¹H NMR (400 MHz, DMSO-d₆) δ 11.05 (s, 1H), 7.79 (s, 3H), 7.55 (d, J=8.4 Hz, 1H), 7.18 (br, s, 1H), 7.01 (s, 1H), 6.90 (d, J=8.4 Hz, 1H), 5.03 (dd, J=5.2 Hz, 12.8 Hz, 1H), 3.61-3.54 (m, 20H), 3.35 (s, 2H), 2.98 (s, 2H), 2.92-2.83 (m, 1H), 2.61-2.54 (m, 2H), 2.02-1.98 (m, 1H).

### Example 65: (2-(2,6-Dioxopiperidin-3-yl)-1,3-dioxoisoindolin-5-yl)glycine (Linker 65)

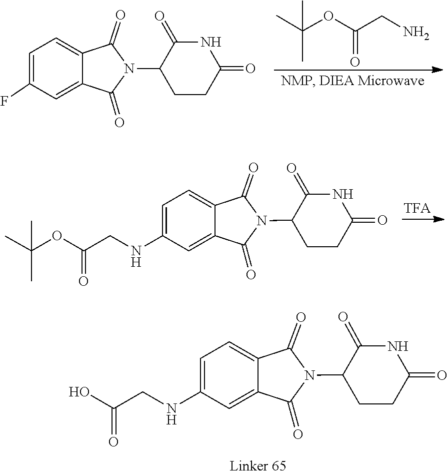

Linker 65 was synthesized following the same procedure as Linker 60 as described in Example 60. (1.0 g, yield: 84%). MS (ESI) m/z=332.0 [M+H]⁺. ¹H NMR (400 MHz, DMSO-d₆) δ 12.80 (br, 1H), 11.06 (s, 1H), 7.59 (d, J=8.4 Hz, 1H), 7.32 (br, s, 1H), 6.98 (d, J=1.2 Hz, 1H), 6.89 (dd, J=2.0 Hz, 8.4 Hz, 1H), 5.04 (dd, J=5.6 Hz, 13.2 Hz, 1H), 4.03 (s, 2H), 2.92-2.83 (m, 1H), 2.60-2.52 (m, 2H), 2.03-1.98 (m, 1H).

### Example 66: 3-((2-(2,6-Dioxopiperidin-3-yl)-1,3-dioxoisoindolin-5-yl)amino)propanoic acid (Linker 66)

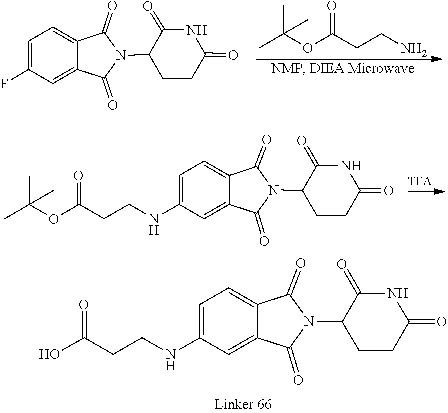

Linker 66 was synthesized following the same procedure as Linker 60 as described in Example 60. (1.24 g, yield: 60%). MS (ESI) m/z=346.0 [M+H]⁺. ¹H NMR (400 MHz, DMSO-d₆) δ 11.05 (s, 1H), 7.57 (d, J=8.4 Hz, 1H), 6.97 (d, J=2.0 Hz, 1H), 6.87 (dd, J=2.0 Hz, 8.4 Hz, 1H), 5.02 (dd, J=5.2 Hz, 12.8 Hz, 1H), 3.41 (t, J=6.8 Hz, 2H), 2.89-2.83 (m, 1H), 2.60-2.52 (m, 4H), 2.02-1.97 (m, 1H).

### Example 67: 4-((2-(2,6-Dioxopiperidin-3-yl)-1,3-dioxoisoindolin-5-yl)amino)butanoic acid (Linker 67)

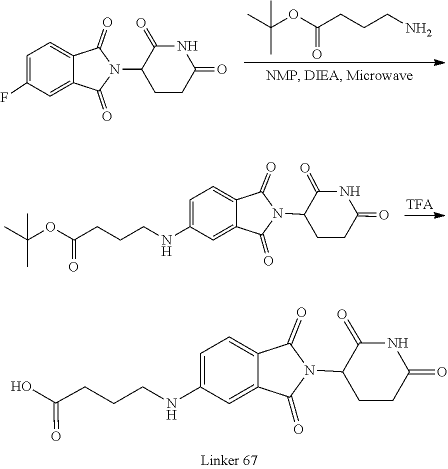 [SMILES: O=C1CCC(N2C(=O)c3ccc(F)cc3C2=O)C(=O)N1 ; CC(C)(C)OC(=O)CCCN ; CC(C)(C)OC(=O)CCCNc1ccc2c(c1)C(=O)N(C1CCC(=O)NC1=O)C2=O ; O=C(O)CCCNc1ccc2c(c1)C(=O)N(C1CCC(=O)NC1=O)C2=O]

Linker 67 was synthesized following the same procedure as Linker 60 as described in Example 60. (0.52 g, yield: 25%). MS (ESI) m/z=360.1 [M+H]⁺. ¹H NMR (400 MHz, DMSO-d₆) δ 12.12 (s, 1H), 11.05 (s, 1H), 7.55 (d, J=8.4 Hz, 1H), 7.14 (t, J=4.8 Hz, 1H), 6.95 (d, J=2.0 Hz, 1H), 6.85 (dd, J=2.0 Hz, 8.4 Hz, 1H), 5.02 (dd, J=5.6 Hz, 12.8 Hz, 1H), 3.21-3.16 (m, 2H), 2.91-2.83 (m, 1H), 2.60-2.51 (m, 2H), 2.34 (t, J=7.2 Hz, 2H), 2.01-1.97 (m, 1H), 1.82-1.75 (m, 2H).

### Example 68: 5-((2-(2,6-Dioxopiperidin-3-yl)-1,3-dioxoisoindolin-5-yl)amino)pentanoic acid (Linker 68)

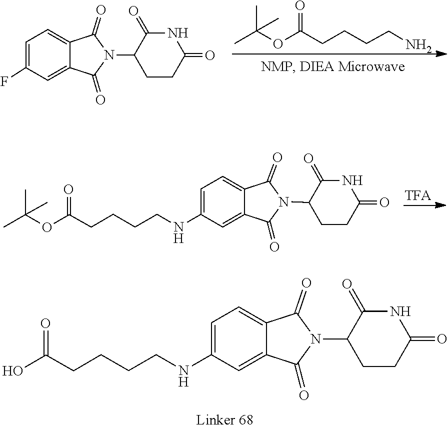

Linker 68 was synthesized following the same procedure as Linker 60 as described in Example 60. (0.66 g, yield: 51%). MS (ESI) m/z=374.1 [M+H]⁺. ¹H NMR (400 MHz, DMSO-d₆) δ 12.03 (br, 1H), 11.05 (s, 1H), 7.55 (d, J=8.4 Hz, 1H), 7.10 (t, J=5.2 Hz, 1H), 6.94 (s, 1H), 6.83 (dd, J=1.6 Hz, 8.4 Hz, 1H), 5.02 (dd, J=5.6 Hz, 12.8 Hz, 1H), 3.17-3.16 (m, 2H), 2.92-2.83 (m, 1H), 2.60-2.53 (m, 2H), 2.26-2.25 (m, 2H), 2.01-1.98 (m, 1H), 1.60-1.59 (m, 4H).

### Example 69: 6-((2-(2,6-Dioxopiperidin-3-yl)-1,3-dioxoisoindolin-5-yl)amino)hexanoic acid (Linker 69)

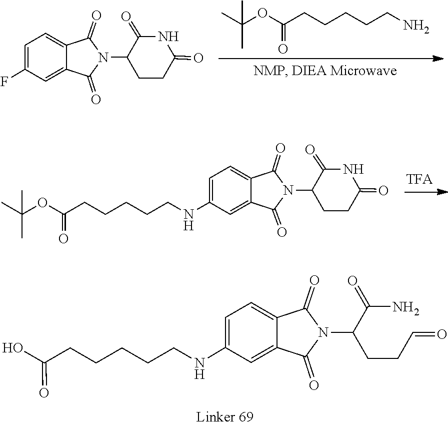 [SMILES: O=C1CCC(N2C(=O)c3ccc(F)cc3C2=O)C(=O)N1 ; CC(C)(C)OC(=O)CCCCCN ; CC(C)(C)OC(=O)CCCCCNc1ccc2c(c1)C(=O)N(C1CCC(=O)NC1=O)C2=O ; NC(=O)C(CCC=O)N1C(=O)c2ccc(NCCCCCC(=O)O)cc2C1=O]

Linker 69 was synthesized following the same procedure as Linker 60 as described in Example 60. (1.33 g, yield: 66%). MS (ESI) m/z=388.1 [M+H]⁺. ¹H NMR (400 MHz, DMSO-d₆) δ 11.98 (s, 1H), 11.05 (s, 1H), 7.55 (d, J=8.4 Hz, 1H), 7.08 (t, J=5.2 Hz, 1H), 6.95 (s, 1H), 6.83 (dd, J=1.2 Hz, 8.4 Hz, 1H), 5.03 (dd, J=5.2 Hz, 12.8 Hz, 1H), 3.17-3.12 (m, 2H), 2.92-2.83 (m, 1H), 2.60-2.53 (m, 2H), 2.22 (t, J=7.2 Hz, 2H), 2.01-1.98 (m, 1H), 1.61-1.51 (m, 4H), 1.41-1.33 (m, 2H).

### Example 70: 7-((2-(2,6-Dioxopiperidin-3-yl)-1,3-dioxoisoindolin-5-yl)amino)heptanoic acid (Linker 70)

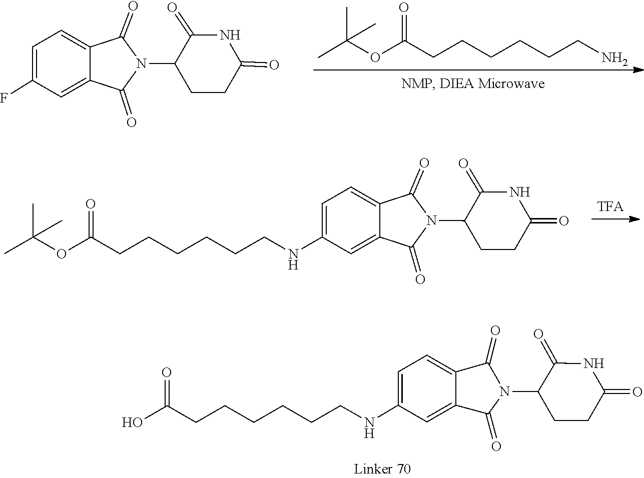

Linker 70 was synthesized following the same procedure as Linker 60 as described in Example 60. (1.06 g, yield: 39%). MS (ESI) m/z=402.1 [M+H]⁺. ¹H NMR (400 MHz, DMSO-d₆) δ 11.94 (s, 1H), 11.04 (s, 1H), 7.55 (d, J=8.4 Hz, 1H), 7.09 (t, J=5.6 Hz, 1H), 6.94 (d, J=2.0 Hz, 1H), 6.84 (dd, J=2.0 Hz, 8.4 Hz, 1H), 5.02 (dd, J=5.6 Hz, 13.2 Hz, 1H), 3.17-3.12 (m, 2H), 2.88-2.83 (m, 1H), 2.60-2.53 (m, 2H), 2.21 (t, J=7.2 Hz, 2H), 2.01-1.97 (m, 1H), 1.58-1.48 (m, 4H), 1.39-1.29 (m, 4H).

### Example 71: 8-((2-(2,6-Dioxopiperidin-3-yl)-1,3-dioxoisoindolin-5-yl)amino)octanoic acid (Linker 71)

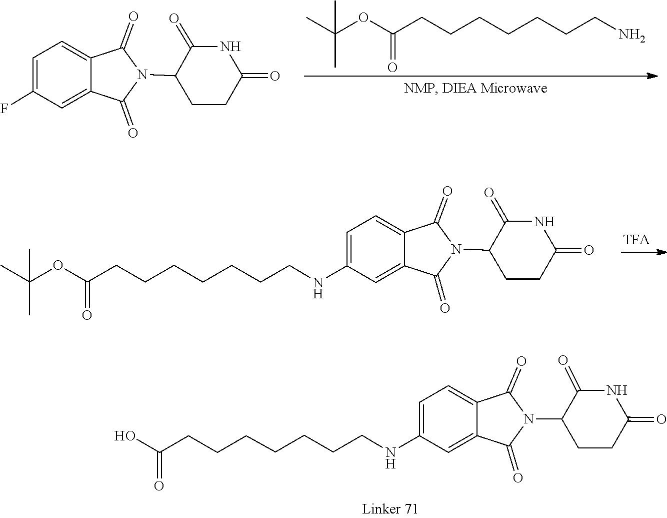 [SMILES: O=C1CCC(N2C(=O)c3ccc(F)cc3C2=O)C(=O)N1 ; CC(C)(C)OC(=O)CCCCCCCN ; CC(C)(C)OC(=O)CCCCCCCNc1ccc2c(c1)C(=O)N(C1CCC(=O)NC1=O)C2=O ; O=C(O)CCCCCCCNc1ccc2c(c1)C(=O)N(C1CCC(=O)NC1=O)C2=O]

Linker 71 was synthesized following the same procedure as Linker 60 as described in Example 60. (1.66 g, yield: 51%). MS (ESI) m/z=416.1 [M+H]⁺. ¹H NMR (400 MHz, DMSO-d₆) δ 11.95 (s, 1H), 11.05 (s, 1H), 7.55 (d, J=8.4 Hz, 1H), 7.09 (t, J=5.6 Hz, 1H), 6.94 (d, J=2.0 Hz, 1H), 6.84 (dd, J=2.0 Hz, 8.4 Hz, 1H), 5.02 (dd, J=5.6 Hz, 13.2 Hz, 1H), 3.17-3.12 (m, 2H), 2.88-2.83 (m, 1H), 2.60-2.53 (m, 2H), 2.19 (t, J=7.2 Hz, 2H), 2.02-1.98 (m, 1H), 1.58-1.47 (m, 4H), 1.36-1.29 (m, 6H).

### Example 72: 5-((2-Aminoethyl)amino)-2-(2,6-dioxopiperidin-3-yl)isoindoline-1,3-dione (Linker 72)

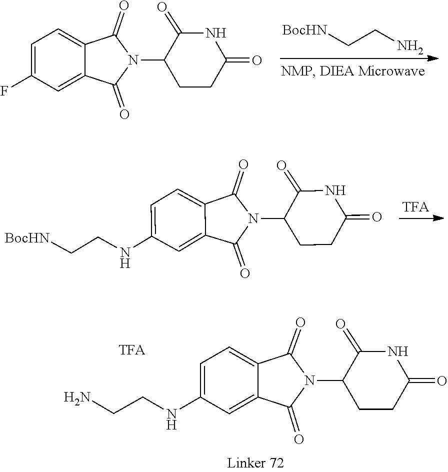 [SMILES: O=C1CCC(N2C(=O)c3ccc(F)cc3C2=O)C(=O)N1 ; CC(C)(C)OC(=O)NCCN ; CC(C)(C)OC(=O)NCCNc1ccc2c(c1)C(=O)N(C1CCC(=O)NC1=O)C2=O ; NCCNc1ccc2c(c1)C(=O)N(C1CCC(=O)NC1=O)C2=O]

Linker 72 was synthesized following the same procedure as Linker 60 as described in Example 60. (1.74 g, yield: 80%). MS (ESI) m/z=317.1 [M+H]⁺. ¹H NMR (400 MHz, DMSO-d₆) δ 11.08 (s, 1H), 8.10 (s, 3H), 7.62 (d, J=8.4 Hz, 1H), 7.33 (t, J=5.2 Hz, 1H), 7.05 (s, 1H), 6.94 (d, J=8.0 Hz, 1H), 5.07 (dd, J=5.2 Hz, 12.8 Hz, 1H), 3.50-3.49 (m, 2H), 3.03 (t, J=6.0 Hz, 2H), 2.95-2.86 (m, 1H), 2.63-2.57 (m, 2H), 2.05-2.02 (m, 1H).

### Example 73: 5-((3-Aminopropyl)amino)-2-(2,6-dioxopiperidin-3-yl)isoindoline-1,3-dione (Linker 73)

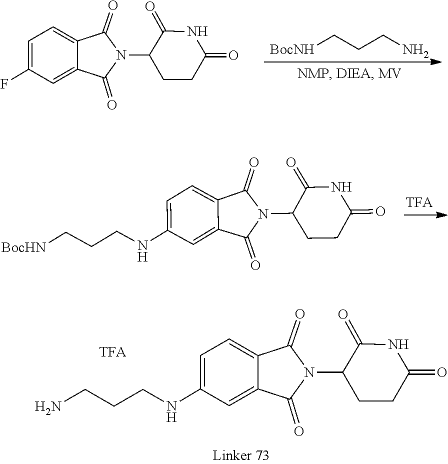 [SMILES: O=C1CCC(N2C(=O)c3ccc(F)cc3C2=O)C(=O)N1 ; CC(C)(C)OC(=O)NCCCN ; CC(C)(C)OC(=O)NCCCNc1ccc2c(c1)C(=O)N(C1CCC(=O)NC1=O)C2=O ; NCCCNc1ccc2c(c1)C(=O)N(C1CCC(=O)NC1=O)C2=O]

Linker 73 was synthesized following the same procedure as Linker 60 as described in Example 60. (1.3 g, yield: 57%). MS (ESI) m/z=331.1 [M+H]⁺. ¹H NMR (400 MHz, DMSO-d₆) δ 11.07 (s, 1H), 7.85 (br, 3H), 7.59 (d, J=8.4 Hz, 1H), 7.22 (t, J=5.2 Hz, 1H), 6.98 (d, J=2.0 Hz, 1H), 6.88 (dd, J=2.0 Hz, 8.4 Hz, 1H), 5.04 (dd, J=5.6 Hz, 13.2 Hz, 1H), 3.29-3.25 (m, 2H), 2.91-2.85 (m, 3H), 2.60-2.53 (m, 2H), 2.02-1.98 (m, 1H), 1.87-1.81 (m, 2H).

### Example 74: 5-((4-Aminobutyl)amino)-2-(2,6-dioxopiperidin-3-yl)isoindoline-1,3-dione (Linker 74)

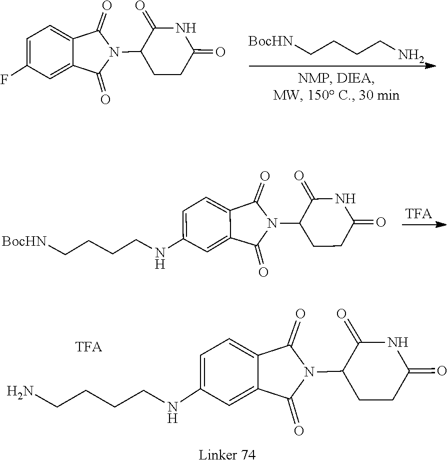 [SMILES: O=C1CCC(N2C(=O)c3ccc(F)cc3C2=O)C(=O)N1 ; CC(C)(C)OC(=O)NCCCCN ; CC(C)(C)OC(=O)NCCCCNc1ccc2c(c1)C(=O)N(C1CCC(=O)NC1=O)C2=O ; NCCCCNc1ccc2c(c1)C(=O)N(C1CCC(=O)NC1=O)C2=O ; "TFA"]

Linker 74 was synthesized following the same procedure as Linker 60 as described in Example 60. (2.9 g, yield: 85%). MS (ESI) m/z=345.1 [M+H]⁺. ¹H NMR (400 MHz, DMSO-d₆) δ 11.08 (s, 1H), 7.97 (br, 3H), 7.58 (d, J=8.4 Hz, 1H), 7.22 (br, s, 1H), 6.99 (s, 1H), 6.89 (d, J=8.0 Hz, 1H), 5.05 (dd, J=5.2 Hz, 12.8 Hz, 1H), 3.22 (s, 2H), 2.93-2.84 (m, 3H), 2.63-2.53 (m, 2H), 2.04-2.00 (m, 1H), 1.66 (s, 4H).

### Example 75: 5-((5-Aminopentyl)amino)-2-(2,6-dioxopiperidin-3-yl)isoindoline-1,3-dione (Linker 75)

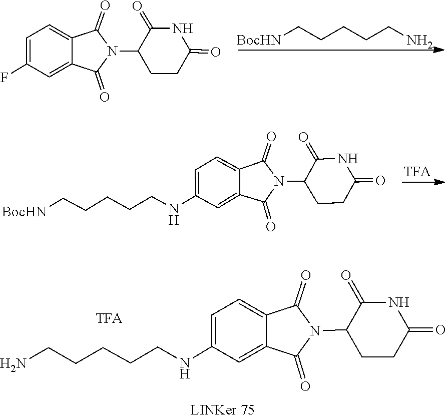 [SMILES: O=C1CCC(N2C(=O)c3ccc(F)cc3C2=O)C(=O)N1 ; CC(C)(C)OC(=O)NCCCCCN ; CC(C)(C)OC(=O)NCCCCCNc1ccc2c(c1)C(=O)N(C1CCC(=O)NC1=O)C2=O ; NCCCCCNc1ccc2c(c1)C(=O)N(C1CCC(=O)NC1=O)C2=O]

Linker 75 was synthesized following the same procedure as Linker 60 as described in Example 60. (1.8 g, yield: 78%). MS (ESI) m/z=359.1 [M+H]⁺. ¹H NMR (400 MHz, DMSO-d₆) δ 11.09 (s, 1H), 7.89 (br, 3H), 7.57 (d, J=6.8 Hz, 1H), 7.17 (br, s, 1H), 6.96 (s, 1H), 6.86 (d, J=6.0 Hz, 1H), 5.05 (d, J=7.2 Hz, 1H), 3.19-3.15 (m, 2H), 2.89-2.70 (m, 3H), 2.61-2.51 (m, 2H), 2.01-1.90 (m, 1H), 1.62-1.56 (m, 4H), 1.45-1.40 (m, 2H).

### Example 76: 5-((6-Aminohexyl)amino)-2-(2,6-dioxopiperidin-3-yl)isoindoline-1,3-dione (Linker 76)

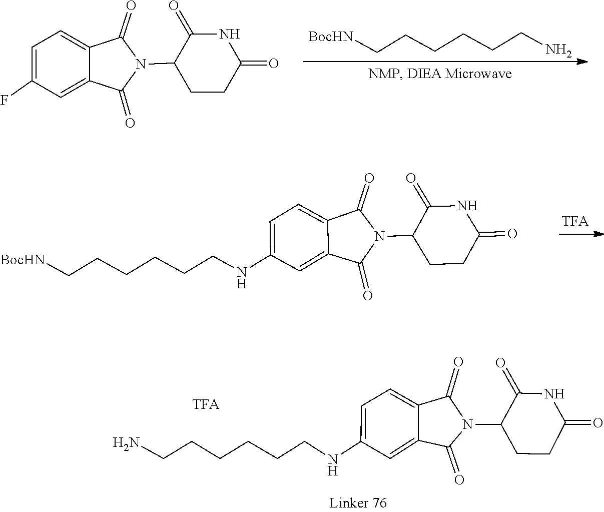 [SMILES: O=C1CCC(N2C(=O)c3ccc(F)cc3C2=O)C(=O)N1 ; CC(C)(C)OC(=O)NCCCCCCN ; CC(C)(C)OC(=O)NCCCCCCNc1ccc2c(c1)C(=O)N(C1CCC(=O)NC1=O)C2=O ; NCCCCCCNc1ccc2c(c1)C(=O)N(C1CCC(=O)NC1=O)C2=O]

Linker 76 was synthesized following the same procedure as Linker 60 as described in Example 60. (1.8 g, yield: 62%). MS (ESI) m/z=373.1 [M+H]⁺. ¹H NMR (400 MHz, DMSO-d₆) δ 11.05 (s, 1H), 7.71 (br, 3H), 7.57 (d, J=8.4 Hz, 1H), 7.12 (t, J=5.2 Hz, 1H), 6.94 (d, J=2.0 Hz, 1H), 6.85 (dd, J=2.0 Hz, 8.4 Hz, 1H), 5.03 (dd, J=5.2 Hz, 12.8 Hz, 1H), 3.17-3.16 (m, 2H), 2.88-2.77 (m, 3H), 2.60-2.53 (m, 2H), 2.01-1.98 (m, 1H), 1.59-1.51 (m, 4H), 1.37-1.36 (m, 4H).

### Example 77: 5-((7-Aminoheptyl)amino)-2-(2,6-dioxopiperidin-3-yl)isoindoline-1,3-dione (Linker 77)

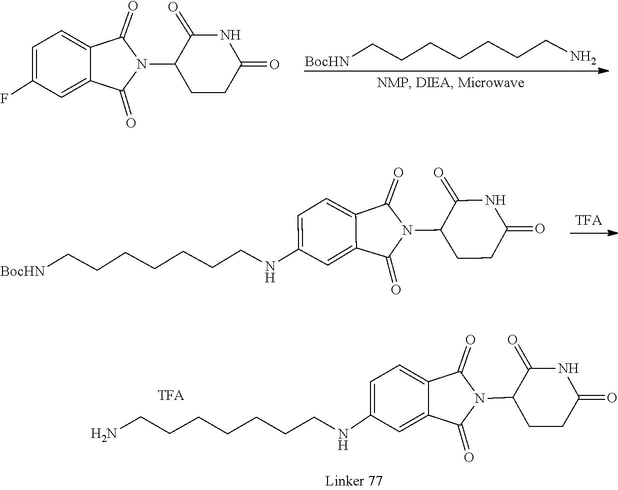 [SMILES: O=C1CCC(N2C(=O)c3ccc(F)cc3C2=O)C(=O)N1 ; CC(C)(C)OC(=O)NCCCCCCCN ; CC(C)(C)OC(=O)NCCCCCCCNc1ccc2c(c1)C(=O)N(C1CCC(=O)NC1=O)C2=O ; NCCCCCCCNc1ccc2c(c1)C(=O)N(C1CCC(=O)NC1=O)C2=O]

Linker 77 was synthesized following the same procedure as Linker 60 as described in Example 60. (1.3 g, yield: 70%). MS (ESI) m/z=387.1 [M+H]⁺. ¹H NMR (400 MHz, DMSO-d₆) δ 11.05 (s, 1H), 7.72 (br, 3H), 7.56 (d, J=8.4 Hz, 1H), 7.12 (t, J=5.6 Hz, 1H), 6.94 (d, J=2.0 Hz, 1H), 6.85 (dd, J=2.4 Hz, 8.8 Hz, 1H), 5.03 (dd, J=5.6 Hz, 12.8 Hz, 1H), 3.18-3.14 (m, 2H), 2.92-2.76 (m, 3H), 2.60-2.51 (m, 2H), 2.01-1.98 (m, 1H), 1.59-1.51 (m, 4H), 1.36-1.32 (m, 6H).

### Example 78: 5-((8-Aminooctyl)amino)-2-(2,6-dioxopiperidin-3-yl)isoindoline-1,3-dione (Linker 78)

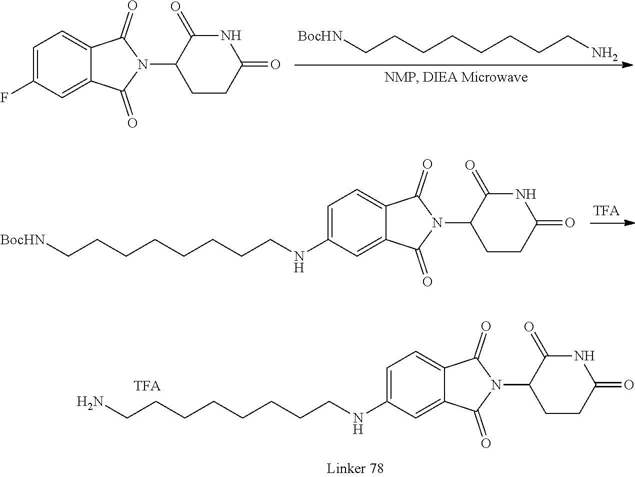 [SMILES: O=C1CCC(N2C(=O)c3ccc(F)cc3C2=O)C(=O)N1 ; CC(C)(C)OC(=O)NCCCCCCCCN ; CC(C)(C)OC(=O)NCCCCCCCCNc1ccc2c(c1)C(=O)N(C1CCC(=O)NC1=O)C2=O ; NCCCCCCCCNc1ccc2c(c1)C(=O)N(C1CCC(=O)NC1=O)C2=O]

Linker 78 was synthesized following the same procedure as Linker 60 as described in Example 60. (1.6 g, yield: 62%). MS (ESI) m/z=401.1 [M+H]⁺. ¹H NMR (400 MHz, DMSO-d₆) δ 11.05 (s, 1H), 7.73 (br, 3H), 7.56 (d, J=8.4 Hz, 1H), 7.14 (br, 1H), 6.94 (d, J=1.6 Hz, 1H), 6.85 (dd, J=2.0 Hz, 8.8 Hz, 1H), 5.03 (dd, J=5.6 Hz, 12.8 Hz, 1H), 3.15 (t, J=7.2 Hz, 2H), 2.89-2.83 (m, 1H), 2.80-2.75 (m, 2H), 2.60-2.54 (m, 2H), 2.02-1.98 (m, 1H), 1.59-1.51 (m, 4H), 1.37-1.30 (m, 8H).

### Example 79: 3-(2-((2-(2,6-Dioxopiperidin-3-yl)-1,3-dioxoisoindolin-5-yl)amino)ethoxy)propanoic acid (Linker 79)

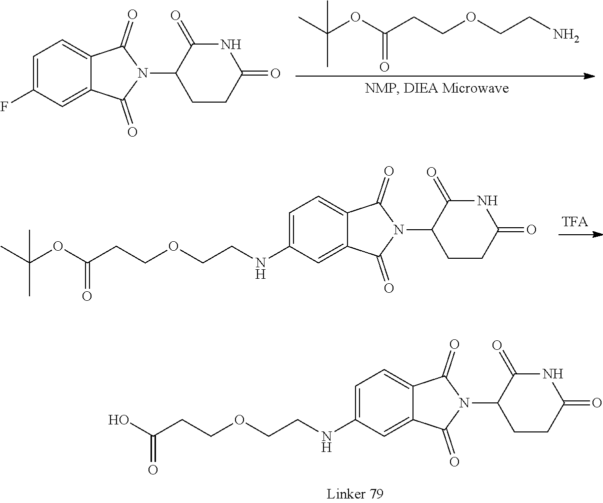 [SMILES: O=C1CCC(N2C(=O)c3ccc(F)cc3C2=O)C(=O)N1 ; CC(C)(C)OC(=O)CCOCCN ; CC(C)(C)OC(=O)CCOCCNc1ccc2c(c1)C(=O)N(C1CCC(=O)NC1=O)C2=O ; O=C(O)CCOCCNc1ccc2c(c1)C(=O)N(C1CCC(=O)NC1=O)C2=O]

Linker 79 was synthesized following the same procedure as Linker 60 as described in Example 60. (1.7 g, yield: 60%). MS (ESI) m/z=390.1 [M+H]⁺. ¹H NMR (400 MHz, DMSO-d₆) δ 12.19 (br, 1H), 11.06 (s, 1H), 7.57 (d, J=8.4 Hz, 1H), 7.09 (br, 1H), 7.01 (d, J=2.0 Hz, 1H), 6.90 (dd, J=2.0 Hz, 8.4 Hz, 1H), 5.04 (dd, J=5.6 Hz, 13.2 Hz, 1H), 3.66 (t, J=6.4 Hz, 2H), 3.59 (t, J=5.6 Hz, 2H), 3.35 (t, J=5.2 Hz, 2H), 2.93-2.84 (m, 1H), 2.62-2.56 (m, 2H), 2.52-2.47 (m, 2H), 2.03-1.99 (m, 1H).

### Example 80: 3-(2-(2-((2-(2,6-Dioxopiperidin-3-yl)-1,3-dioxoisoindolin-5-yl)amino)ethoxy)ethoxy)propanoic acid (Linker 80)

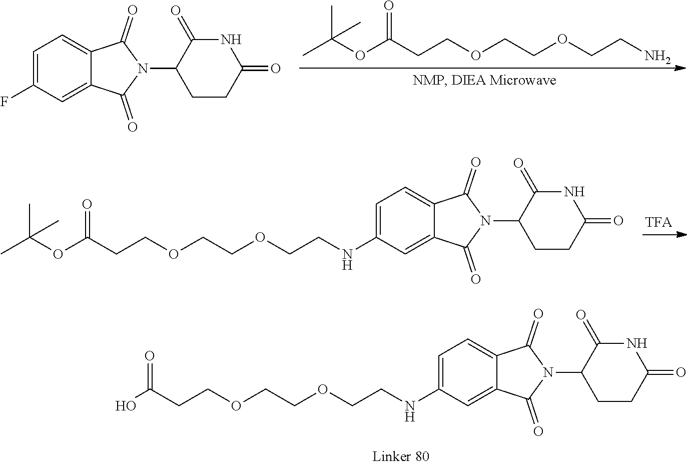

Linker 80 was synthesized following the same procedure as Linker 60 as described in Example 60. (2.3 g, yield: 78%). MS (ESI) m/z=434.1 [M+H]⁺. ¹H NMR (400 MHz, DMSO-d₆) δ 11.06 (s, 1H), 7.57 (d, J=8.4 Hz, 1H), 7.02 (d, J=2.0 Hz, 1H), 6.90 (dd, J=2.0 Hz, 8.4 Hz, 1H), 5.04 (dd, J=5.6 Hz, 13.2 Hz, 1H), 3.63-3.59 (m, 4H), 3.57-3.51 (m, 4H), 3.36 (t, J=5.6 Hz, 2H), 2.90-2.84 (m, 1H), 2.61-2.55 (m, 2H), 2.44 (t, J=6.4 Hz, 2H), 2.04-1.99 (m, 1H).

### Example 81: 3-(2-(2-(2-((2-(2,6-Dioxopiperidin-3-yl)-1,3-dioxoisoindolin-5-yl)amino)ethoxy)ethoxy)ethoxy)propanoic acid (Linker 81)

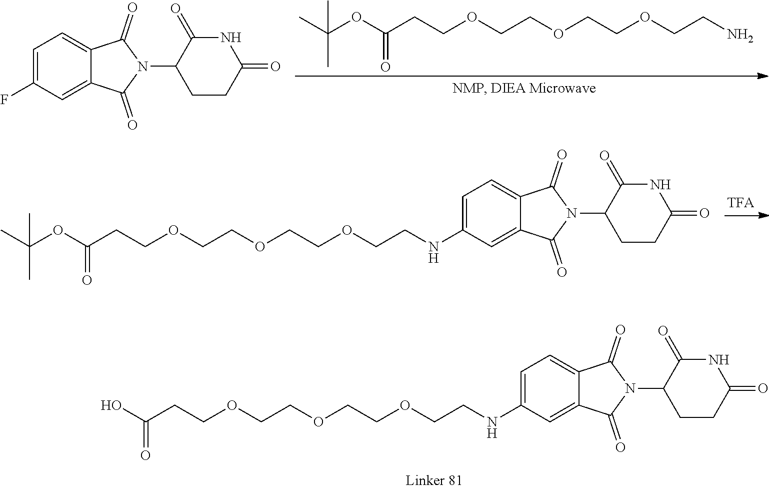 [SMILES: O=C1CCC(N2C(=O)c3ccc(F)cc3C2=O)C(=O)N1 ; CC(C)(C)OC(=O)CCOCCOCCOCCN ; CC(C)(C)OC(=O)CCOCCOCCOCCNc1ccc2c(c1)C(=O)N(C1CCC(=O)NC1=O)C2=O ; O=C(O)CCOCCOCCOCCNc1ccc2c(c1)C(=O)N(C1CCC(=O)NC1=O)C2=O]

Linker 81 was synthesized following the same procedure as Linker 60 as described in Example 60. (1.2 g, yield: 52%). MS (ESI) m/z=478.1 [M+H]⁺. ¹H NMR (400 MHz, DMSO-d₆) δ 7.59 (d, J=11.2 Hz, 1H), 7.23 (t, J=6.8 Hz, 1H), 7.04 (d, J=1.6 Hz, 1H), 7.04 (dd, J=2.4 Hz, 11.2 Hz, 1H), 5.06 (dd, J=7.2 Hz, 16.8 Hz, 1H), 3.64-3.57 (m, 8H), 3.54-3.48 (m, 4H), 3.40-3.38 (m, 2H), 2.92-2.89 (m, 1H), 2.64-2.54 (m, 2H), 2.42-2.38 (m, 2H), 2.05-2.01 (m, 1H).

### Example 82: 1-((2-(2,6-Dioxopiperidin-3-yl)-1,3-dioxoisoindolin-5-yl)amino)-3,6,9,12-tetraoxapentadecan-15-oic acid (Linker 82)

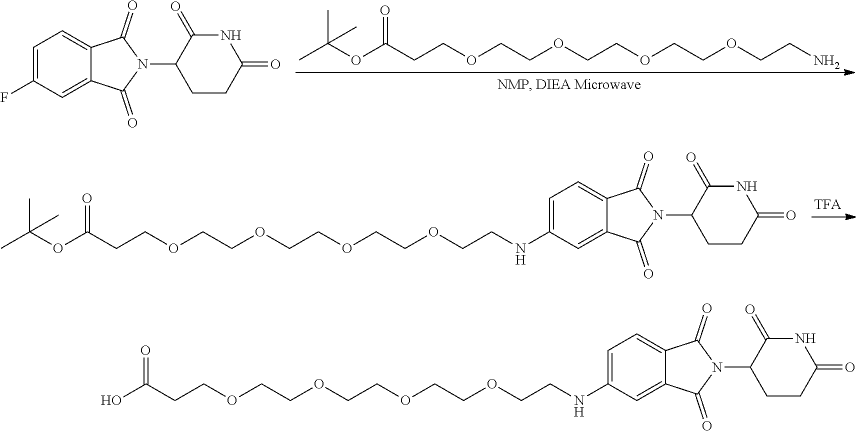

Linker 82 was synthesized following the same procedure as Linker 60 as described in Example 60. (1.3 g, yield: 55%). MS (ESI) m/z=522.1 [M+H]⁺. ¹H NMR (400 MHz, DMSO-d₆) δ 12.17 (br, 1H), 11.07 (s, 1H), 7.56 (d, J=8.4 Hz, 1H), 7.17 (t, J=5.6 Hz, 1H), 7.01 (d, J=1.2 Hz, 1H), 6.90 (dd, J=1.6 Hz, 8.4 Hz, 1H), 5.03 (dd, J=5.6 Hz, 12.8 Hz, 1H), 3.61-3.48 (m, 18H), 2.92-2.83 (m, 1H), 2.60-2.54 (m, 2H), 2.43 (t, J=6.4 Hz, 2H), 2.03-1.98 (m, 1H).

### Example 83: 1-((2-(2,6-Dioxopiperidin-3-yl)-1,3-dioxoisoindolin-5-yl)amino)-3,6,9,12,15-pentaoxaoctadecan-18-oic acid (Linker 83)

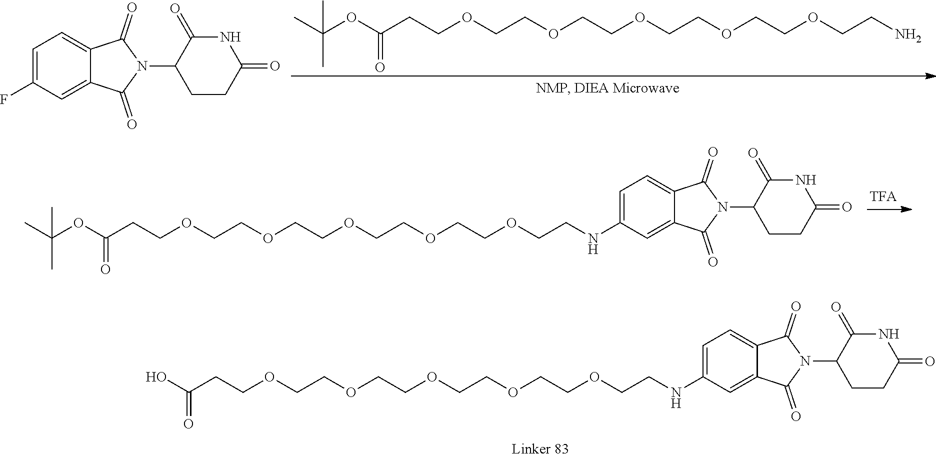 [SMILES: O=C1CCC(N2C(=O)c3ccc(F)cc3C2=O)C(=O)N1 ; CC(C)(C)OC(=O)CCOCCOCCOCCOCCOCCN ; CC(C)(C)OC(=O)CCOCCOCCOCCOCCOCCNc1ccc2c(c1)C(=O)N(C1CCC(=O)NC1=O)C2=O ; O=C(O)CCOCCOCCOCCOCCOCCNc1ccc2c(c1)C(=O)N(C1CCC(=O)NC1=O)C2=O]

Linker 83 was synthesized following the same procedure as Linker 60 as described in Example 60. (1.0 g, yield: 50%). MS (ESI) m/z=566.1 [M+H]⁺. ¹H NMR (400 MHz, DMSO-d₆) δ 12.17 (br, s, 1H), 11.07 (s, 1H), 7.56 (d, J=8.0 Hz, 1H), 7.17 (t, J=5.6 Hz, 1H), 7.01 (s, 1H), 6.90 (dd, J=1.6 Hz, 8.4 Hz, 1H), 5.03 (dd, J=5.6 Hz, 13.2 Hz, 1H), 3.60-3.48 (m, 22H), 2.89-2.83 (m, 1H), 2.60-2.54 (m, 2H), 2.43 (t, J=6.4 Hz, 2H), 2.01-1.98 (m, 1H).

Procedures for the Synthesis of P300 Binders for P300 PROTACs

### Example 84. Synthesis of 3-(7-(difluoromethyl)-6-(1-methyl-1H-pyrazol-4-yl)-3,4-dihydroquinolin-1(2H)-yl)-N-methyl-1-(piperidin-4-yl)-1,4,6,7-tetrahydro-5H-pyrazolo[4,3-c]pyridine-5-carboxamide (P300 binder 1)

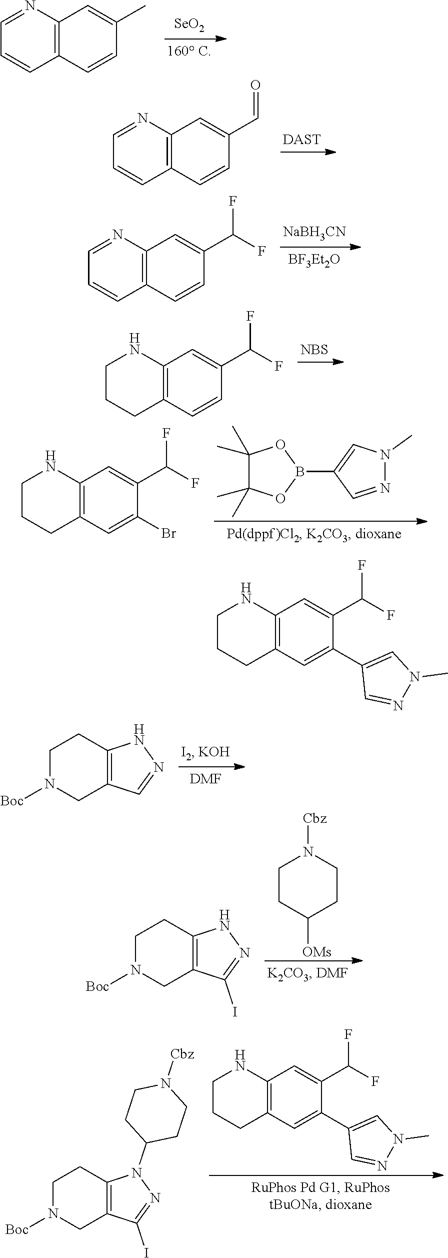

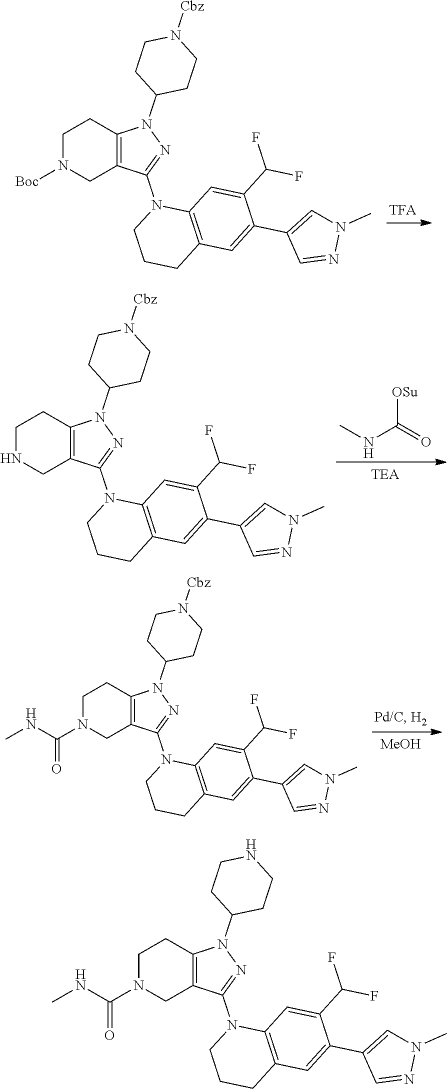

Step 1: Synthesis of quinoline-7-carbaldehyde

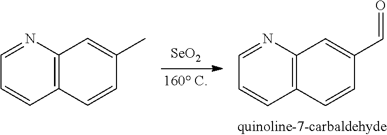

To a solution of 7-methylquinoline (235.0 g, 1.64 mol) at 160° C. was added SeO₂ (220 g, 1.97 mol) portionwise over 25 min. The mixture was stirred at 160° C. for 8 h. After cooling to room temperature, DCM (2000 mL) was added and the mixture was filtered through a pad of Celite. The organic layer was concentrated in vacuo and the crude residue was purified by silica gel chromatography (petroleum ether/EtOAc=10:1) to give quinoline-7-carbaldehyde (100 g, yield: 38%) as a yellow solid.

### Step 2: Synthesis of 7-(difluoromethyl)quinoline

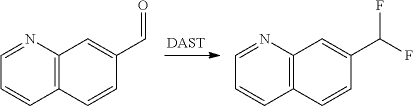

To a cooled (0° C.) solution of quinoline-7-carbaldehyde (35.0 g, 223 mmol) in DCM (400 mL) was added diethylaminosulfurtrifluoride (162.0 g, 1150 mmol) dropwise over 30 min. The mixture was stirred at room temperature for 16 h, before being poured into sat. aq NaHCO₃(2 L) at 0° C. and extracted with DCM (400 mL×2). The combined organic layers were dried over anhydrous Na₂SO₄, filtered, and concentrated in vacuo. The crude residue was purified by silica gel chromatography (petroleum ether/EtOAc=5:1) to give 7-(difluoromethyl)quinolone (26.0 g, yield: 65%) as a yellow oil.

### Step 3: Synthesis of 7-(difluoromethyl)-1,2,3,4-tetrahydroquinoline

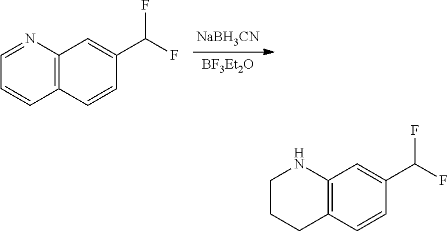

To a cooled (0° C.) solution of 7-(difluoromethyl)quinolone (26.0 g, 72.6 mmol) and NaBH₃CN (46.1 g, 726 mmol) in MeOH (300 mL) was added boron trifluoride diethyl etherate (41.2 g, 290 mmol) dropwise over 20 min. The mixture was heated to 90° C. for 24 h. After cooling to room temperature, the mixture was poured into sat. aq. NaHCO₃(2 L) at 0° C. and extracted with DCM (500 mL×2). The combined organic layers were dried over Na₂SO₄, filtered, and concentrated in vacuo. The crude residue was purified by silica gel chromatography (petroleum ether/EtOAc=20:1) to give 7-(difluoromethyl)-1,2,3,4-tetrahydroquinoline (13.0 g, yield: 49%) as a brown oil.

### Step 4: Synthesis of 6-bromo-7-(difluoromethyl)-1,2,3,4-tetrahydroquinoline

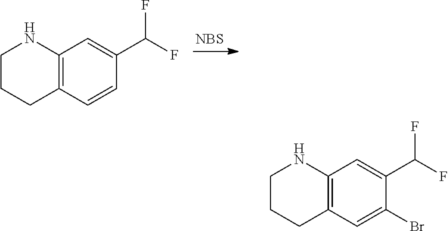

To a solution of 7-(difluoromethyl)-1,2,3,4-tetrahydroquinoline (29.0 g, 158.5 mmol) in DCM (600 mL) at 0° C. was added N-bromosuccinimide (6.90 g, 38.3 mmol) portionwise over 20 min. The mixture was stirred at room temperature for 16 h, then poured into water (100 mL) and extracted with DCM (400 mL×2). The combined organic layers were dried over Na₂SO₄, filtered, and concentrated in vacuo. The crude residue was purified by silica gel chromatography (petroleum ether/EtOAc=300:1) to give 6-bromo-7-(difluoromethyl)-1,2,3,4-tetrahydroquinoline (22.0 g, yield: 52.8%) as a white solid. ¹HNMR (400 MHz, CDCl₃) δ 7.12 (s, 1H), 6.77 (t, J=55.2 Hz, 1H), 6.77 (s, 1H), 4.01 (s, 1H), 3.30 (t, J=6.4 Hz, 2H), 2.74 (t, J=6.0 Hz, 2H), 1.94-1.88 (m, 2H).

### Step 5: Synthesis of 7-(difluoromethyl)-6-(1-methyl-1H-pyrazol-4-yl)-1,2,3,4-tetrahydroquinoline

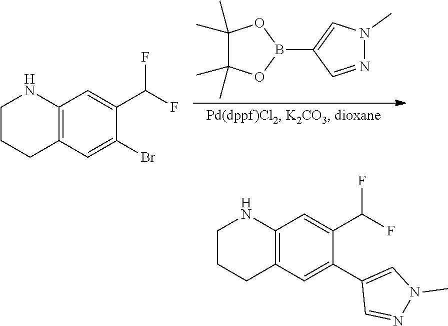

To a solution of 6-bromo-7-(difluoromethyl)-1,2,3,4-tetrahydroquinoline (2 g, 7.66 mmol) and 1-methyl-4-(4,4,5,5-tetramethyl-1,3,2-dioxaborolan-2-yl)-1H-pyrazole (1.59 g, 7.66 mmol) in 1,4-dixoane (50 mL) were added Pd(dppf)Cl₂ (1.6 g, 2.3 mmol), K₂CO₃ (2.11 g, 15.32 mmol). The reaction mixture was heated to 95° C. overnight, then diluted in ethyl acetate, washed with water and brine. The organic layer was concentrated in vacuo and the residue was purified by column (petroleum ether:ethyl acetate=5:1) to afforded 7-(difluoromethyl)-6-(1-methyl-1H-pyrazol-4-yl)-1,2,3,4-tetrahydroquinoline (1.4 g, yield: 69%) as a white solid. MS (ESI) m/z: 264.4 [M+H]⁺.

### Step 6: Synthesis of tert-butyl 3-iodo-1,4,6,7-tetrahydro-5H-pyrazolo[4,3-c]pyridine-5-carboxylate

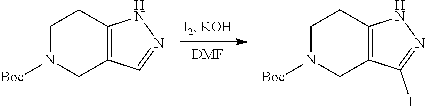

To a solution of tert-butyl 1,4,6,7-tetrahydro-5H-pyrazolo[4,3-c]pyridine-5-carboxylate (10 g, 44.84 mmol) in DMF (100 mL) were added 12 (22.76 g, 89.68 mmol) and KOH (10.04 g, 179.36 mmol). The resulting mixture was stirred at 50° C. overnight. The reaction was quenched with aq. Na₂SO₃ and extracted with EtOAc. The organic layer was dried over Na₂SO₄, filtered and concentrated. The residue was purified by silica gel chromatography (petroleum ether:EtOAc=3:1) to give desired product (8.0 g, yield: 51%) as a colorless oil. MS (ESI) m/z: 350.2 [M+H]⁺.

### Step 7: Synthesis of tert-butyl 1-(1-((benzyloxy)carbonyl)piperidin-4-yl)-3-iodo-1,4,6,7-tetrahydro-5H-pyrazolo[4,3-c]pyridine-5-carboxylate

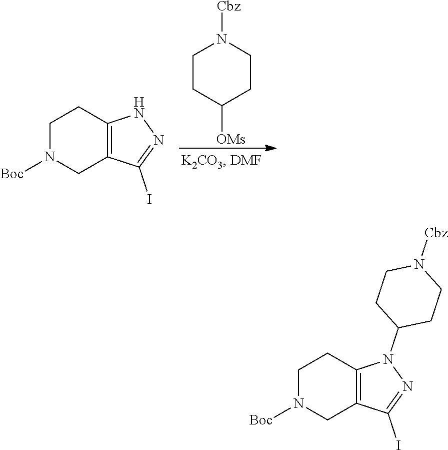 [SMILES: CC(C)(C)OC(=O)N1CCc2[nH]nc(I)c2C1 ; CS(=O)(=O)OC1CCN(C(=O)OCc2ccccc2)CC1 ; CC(C)(C)OC(=O)N1CCc2c(c(I)nn2C2CCN(C(=O)OCc3ccccc3)CC2)C1]

To a solution of tert-butyl 3-iodo-1,4,6,7-tetrahydro-5H-pyrazolo[4,3-c]pyridine-5-carboxylate (6 g, 17.19 mmol) in DMF (50 mL) were added benzyl 4-((methylsulfonyl)oxy)piperidine-1-carboxylate (8.07 g, 25.79 mmol) and K₂CO₃ (4.74 g, 34.38 mmol). The resulting mixture was stirred at 100° C. overnight. After cooling to room temperature, the mixture was diluted with water, extracted with EtOAc. The combined organic phase was washed with brine, dried over Na₂SO₄, filtered and concentrated. The residue was purified by silica gel chromatography (petroleum ether:EtOAc=1:1) to give desired product (4.0 g, yield: 41%) as a white solid. MS (ESI) m/z: 567.4 [M+H]⁺.

### Step 8: Synthesis of tert-butyl 1-(1-((benzyloxy)carbonyl)piperidin-4-yl)-3-(7-(difluoromethyl)-6-(1-methyl-1H-pyrazol-4-yl)-3,4-dihydroquinolin-1(2H)-yl)-1,4,6,7-tetrahydro-5H-pyrazolo[4,3-c]pyridine-5-carboxylate

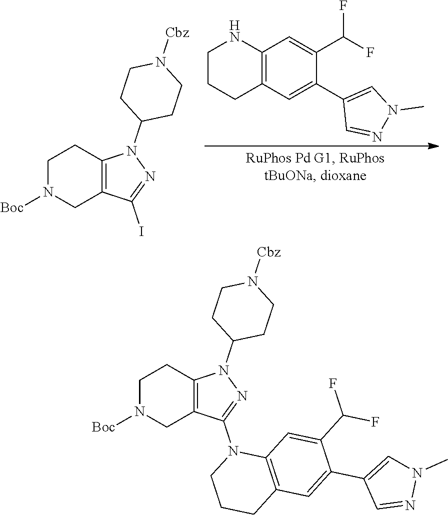

To a solution of tert-butyl 1-(1-((benzyloxy)carbonyl)piperidin-4-yl)-3-iodo-1,4,6,7-tetrahydro-5H-pyrazolo[4,3-c]pyridine-5-carboxylate (132 mg, 0.233 mmol) and 7-(difluoromethyl)-6-(1-methyl-1H-pyrazol-4-yl)-1,2,3,4-tetrahydroquinoline (74 mg, 0.280 mmol) in dioxane (3 mL) were added RuPhos Pd G1 (22.8 mg, 0.028 mmol), RuPhos (13.0 mg, 0.028 mmol) and ^{t}BuONa (78.3 mg, 0.816 mmol). The resulting mixture was stirred at reflux overnight. The reaction mixture was purified by reverse phase flash chromatography to give desired product (80 mg, yield: 49%) as a white solid. MS (ESI) m/z: 703.1 [M+H]⁺.

### Step 9: Synthesis of benzyl 4-(3-(7-(difluoromethyl)-6-(1-methyl-1H-pyrazol-4-yl)-3,4-dihydroquinolin-1(2H)-yl)-4,5,6,7-tetrahydro-1H-pyrazolo[4,3-c]pyridin-1-yl)piperidine-1-carboxylate

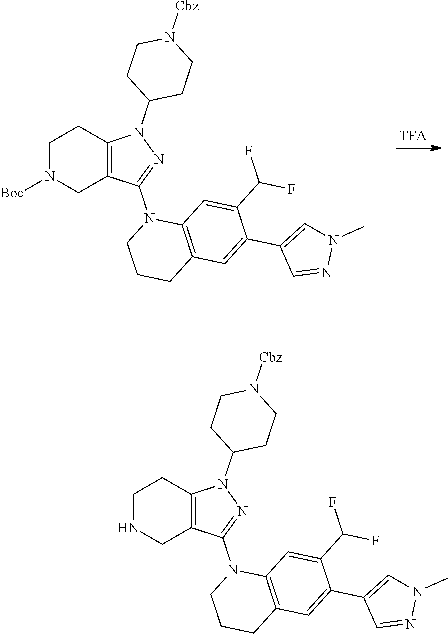 [SMILES: Cn1cc(-c2cc3c(cc2C(F)F)N(c2nn(C4CCN(C(=O)OCc5ccccc5)CC4)c4c2CN(C(=O)OC(C)(C)C)CC4)CCC3)cn1 ; Cn1cc(-c2cc3c(cc2C(F)F)N(c2nn(C4CCN(C(=O)OCc5ccccc5)CC4)c4c2CNCC4)CCC3)cn1]

The mixture of tert-butyl 1-(1-((benzyloxy)carbonyl)piperidin-4-yl)-3-(7-(difluoromethyl)-6-(1-methyl-1H-pyrazol-4-yl)-3,4-dihydroquinolin-1(2H)-yl)-1,4,6,7-tetrahydro-5H-pyrazolo[4,3-c]pyridine-5-carboxylate (189 mg, 0.27 mmol) in DCM:TFA=1:1 (10 ml) was stirred at room temperature for 3 h, before it was concentrated. The residue was used directly in the next step.

### Step 10: Synthesis of benzyl 4-(3-(7-(difluoromethyl)-6-(1-methyl-1H-pyrazol-4-yl)-3,4-dihydroquinolin-1(2H)-yl)-5-(methylcarbamoyl)-4,5,6,7-tetrahydro-1H-pyrazolo[4,3-c]pyridin-1-yl)piperidine-1-carboxylate

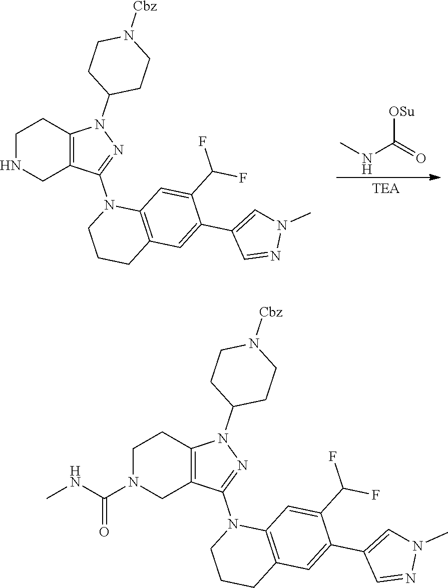

To the solution of benzyl 4-(3-(7-(difluoromethyl)-6-(1-methyl-1H-pyrazol-4-yl)-3,4-dihydroquinolin-1(2H)-yl)-4,5,6,7-tetrahydro-1H-pyrazolo[4,3-c]pyridin-1-yl)piperidine-1-carboxylate (crude product from above reaction) in DCM (10 ml) were added 2,5-dioxopyrrolidin-1-yl methylcarbamate (141 mg, 0.81 mmol) and TEA (82 mg, 0.81 mmol). The resulting mixture was stirred at room temperature for 5 h, before the reaction mixture was purified by reverse phase flash chromatography to give desired product (105 mg, yield: 59%) as a white solid. MS (ESI) m/z: 659.9 [M+H]⁺.

### Step 11: Synthesis of 3-(7-(difluoromethyl)-6-(1-methyl-1H-pyrazol-4-yl)-3,4-dihydroquinolin-1(2H)-yl)-N-methyl-1-(piperidin-4-yl)-1,4,6,7-tetrahydro-5H-pyrazolo[4,3-c]pyridine-5-carboxamide (P300 binder 1)

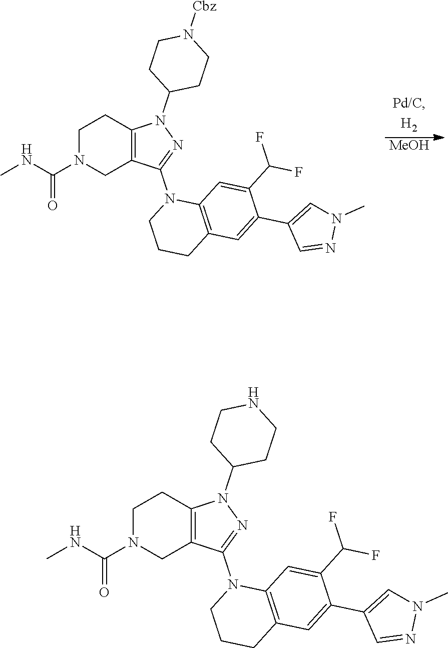

The mixture of benzyl 4-(3-(7-(difluoromethyl)-6-(1-methyl-1H-pyrazol-4-yl)-3,4-dihydroquinolin-1(2H)-yl)-5-(methylcarbamoyl)-4,5,6,7-tetrahydro-1H-pyrazolo[4,3-c]pyridin-1-yl)piperidine-1-carboxylate (105 mg, 0.16 mmol) and Pd/C (10%, 100 mg) in MeOH (10 ml) was stirred under H₂ for 8 h. The reaction mixture was filtered through celite and the filtrate was concentrated to give desired product (56 mg, yield: 67%) as a white solid. MS (ESI) m/z: 525.8 [M+H]⁺.

### Example 85. Synthesis of 2-(4-(7-(difluoromethyl)-1-(5-(methylcarbamoyl)-1-(tetrahydro-2H-pyran-4-yl)-4,5,6,7-tetrahydro-1H-pyrazolo[4,3-c]pyridin-3-yl)-1,2,3,4-tetrahydroquinolin-6-yl)-1H-pyrazol-1-yl)acetic acid (P300 binder 2)

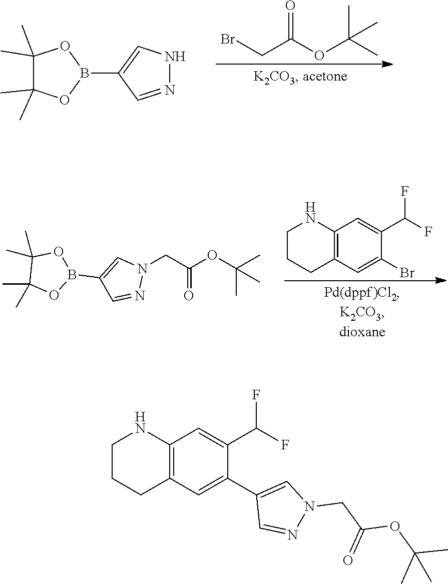

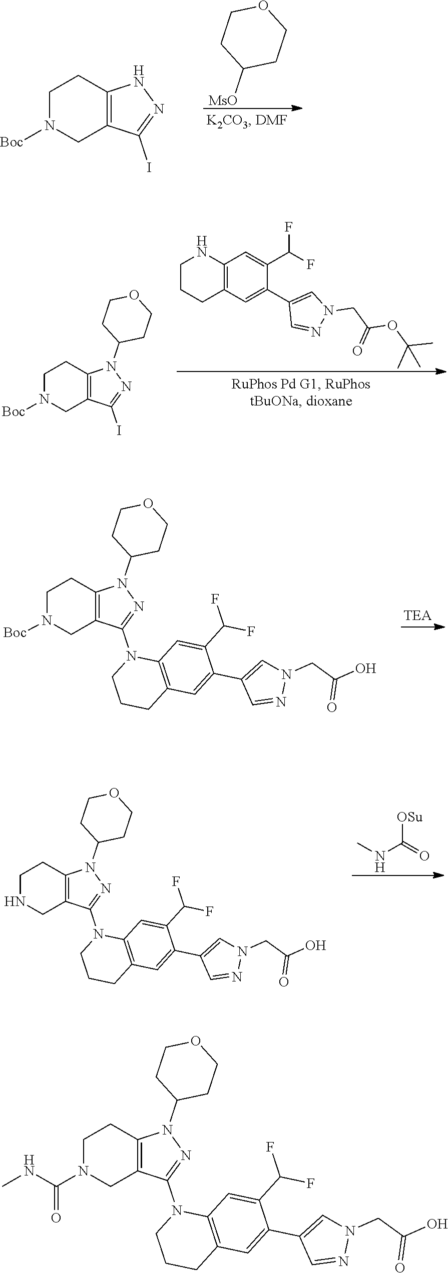

### Step 1: Synthesis of tert-butyl 2-(4-(4,4,5,5-tetramethyl-1,3,2-dioxaborolan-2-yl)-1H-pyrazol-1-yl)acetate

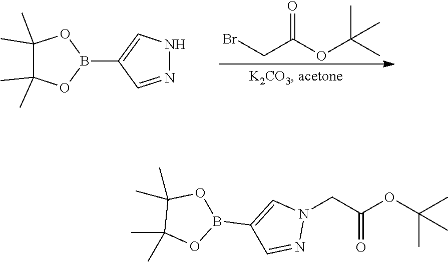

The mixture of 4-(4,4,5,5-tetramethyl-1,3,2-dioxaborolan-2-yl)-1H-pyrazole (2.0 g, 10.31 mmol), tert-butyl 2-bromoacetate (2.21 g, 11.34 mmol) and K₂CO₃ (1.71 g, 12.37 mmol) in acetone (20 ml) was stirred at 65° C. overnight. The reaction mixture was poured into ice water and extracted with EtOAc. The combined organic phase was washed with brine, dried over Na₂SO₄, filtered and concentrated. The residue was purified by silica gel chromatography (PE/EA=5:1) to give desired product (1.7 g, yield: 54%) as an oil. MS (ESI) m/z: 309.2 [M+H]⁺.

### Step 2: Synthesis of tert-butyl 2-(4-(7-(difluoromethyl)-1,2,3,4-tetrahydroquinolin-6-yl)-1H-pyrazol-1-yl)acetate

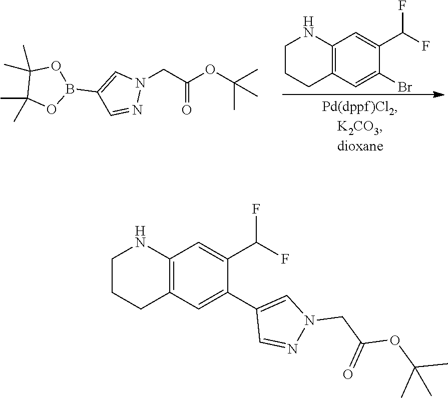

To a solution of 6-bromo-7-(difluoromethyl)-1,2,3,4-tetrahydroquinoline (1.44 g, 5.52 mmol) and tert-butyl 2-(4-(4,4,5,5-tetramethyl-1,3,2-dioxaborolan-2-yl)-1H-pyrazol-1-yl)acetate (1.7 g, 5.52 mmol) in 1,4-dixoane (50 mL) were added Pd(dppf)Cl₂ (1.15 g, 1.66 mmol), K₂CO₃ (1.52 g, 11.03 mmol). The reaction mixture was heated to 95° C. overnight, then diluted in ethyl acetate, washed with water and brine. The organic layer was concentrated in vacuo and the residue was purified by column (petroleum ether:ethyl acetate=5:1) to give desired product (0.9 g, yield: 45%) as an white solid. MS (ESI) m/z: 364.6 [M+H]⁺.

### Step 3: Synthesis of tert-butyl 3-iodo-1-(tetrahydro-2H-pyran-4-yl)-1,4,6,7-tetrahydro-5H-pyrazolo[4,3-c]pyridine-5-carboxylate

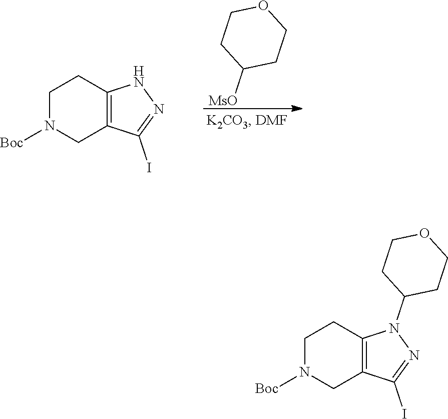 [SMILES: CC(C)(C)OC(=O)N1CCc2[nH]nc(I)c2C1 ; CS(=O)(=O)OC1CCOCC1 ; CC(C)(C)OC(=O)N1CCc2c(c(I)nn2C2CCOCC2)C1]

To a solution of tert-butyl 3-iodo-1,4,6,7-tetrahydro-5H-pyrazolo[4,3-c]pyridine-5-carboxylate (6 g, 17.19 mmol) in DMF (50 mL) were added tetrahydro-2H-pyran-4-yl methanesulfonate (4.64 g, 25.79 mmol) and K₂CO₃ (4.74 g, 34.38 mmol). The resulting mixture was stirred at 100° C. overnight. After cooling to room temperature, the mixture was diluted with water, extracted with EA. The combined organic phase was washed with brine, dried over Na₂SO₄, filtered and concentrated. The residue was purified by silica gel chromatography (PE/EA=1:1) to give desired product (5.0 g, yield: 67%) as a white solid. MS (ESI) m/z: 434.6 [M+H]⁺.

### Step 4: Synthesis of 2-(4-(1-(5-(tert-butoxycarbonyl)-1-(tetrahydro-2H-pyran-4-yl)-4,5,6,7-tetrahydro-1H-pyrazolo[4,3-c]pyridin-3-yl)-7-(difluoromethyl)-1,2,3,4-tetrahydroquinolin-6-yl)-1H-pyrazol-1-yl)acetic acid

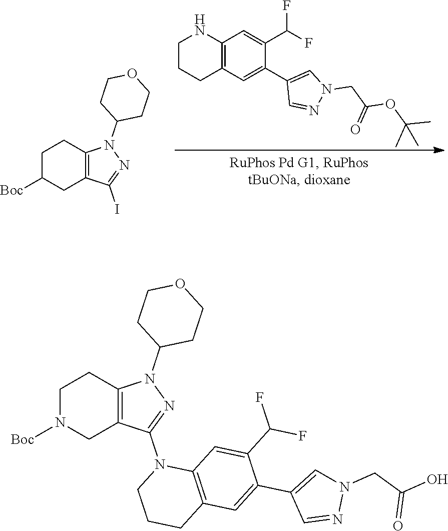 [SMILES: CC(C)(C)OC(=O)C1CCc2c(c(I)nn2C2CCOCC2)C1 ; CC(C)(C)OC(=O)Cn1cc(-c2cc3c(cc2C(F)F)NCCC3)cn1 ; CC(C)(C)OC(=O)N1CCc2c(c(N3CCCc4cc(-c5cnn(CC(=O)O)c5)c(C(F)F)cc43)nn2C2CCOCC2)C1]

To a solution of tert-butyl 3-iodo-1-(tetrahydro-2H-pyran-4-yl)-1,4,6,7-tetrahydro-5H-pyrazolo[4,3-c]pyridine-5-carboxylate (258 mg, 0.60 mmol) and tert-butyl 2-(4-(7-(difluoromethyl)-1,2,3,4-tetrahydroquinolin-6-yl)-1H-pyrazol-1-yl)acetate (262 mg, 0.72 mmol) in dioxane (10 mL) were added RuPhos Pd G1 (58.6 mg, 0.072 mmol), RuPhos (33.4 mg, 0.072 mmol) and ^{t}BuONa (201.3 mg, 2.098 mmol). The resulting mixture was stirred at reflux overnight. The reaction mixture was purified by reverse phase flash chromatography to give desired product (108 mg, yield: 29%) as a white solid. MS (ESI) m/z: 613.7 [M+H]⁺.

### Step 5: Synthesis of 2-(4-(7-(difluoromethyl)-1-(1-(tetrahydro-2H-pyran-4-yl)-4,5,6,7-tetrahydro-1H-pyrazolo[4,3-c]pyridin-3-yl)-1,2,3,4-tetrahydroquinolin-6-yl)-1H-pyrazol-1-yl)acetic acid

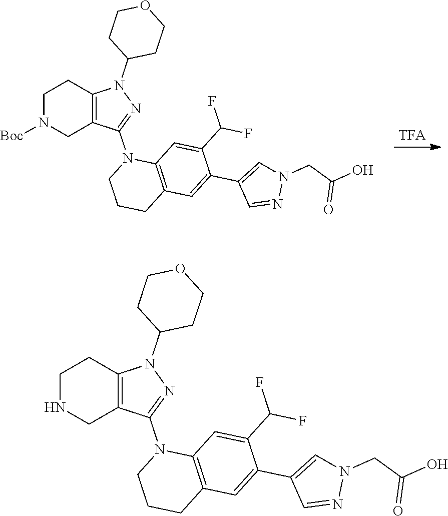 [SMILES: CC(C)(C)OC(=O)N1CCc2c(c(N3CCCc4cc(-c5cnn(CC(=O)O)c5)c(C(F)F)cc43)nn2C2CCOCC2)C1 ; O=C(O)Cn1cc(-c2cc3c(cc2C(F)F)N(c2nn(C4CCOCC4)c4c2CNCC4)CCC3)cn1]

The mixture of 2-(4-(1-(5-(tert-butoxycarbonyl)-1-(tetrahydro-2H-pyran-4-yl)-4,5,6,7-tetrahydro-1H-pyrazolo[4,3-c]pyridin-3-yl)-7-(difluoromethyl)-1,2,3,4-tetrahydroquinolin-6-yl)-1H-pyrazol-1-yl)acetic acid (108 mg, 0.176 mmol) in DCM/TFA=1:1 (6 ml) was stirred at room temperature for 3 h, then it was concentrated and the residue was used directly in the next step.

### Step 6: Synthesis of 2-(4-(7-(difluoromethyl)-1-(5-(methylcarbamoyl)-1-(tetrahydro-2H-pyran-4-yl)-4,5,6,7-tetrahydro-1H-pyrazolo[4,3-c]pyridin-3-yl)-1,2,3,4-tetrahydroquinolin-6-yl)-1H-pyrazol-1-yl)acetic acid (P300 binder 2)

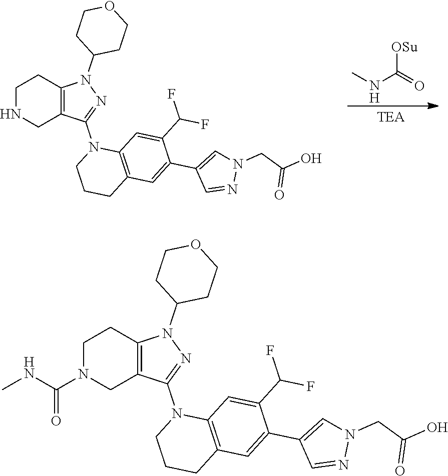

To the solution of 2-(4-(7-(difluoromethyl)-1-(1-(tetrahydro-2H-pyran-4-yl)-4,5,6,7-tetrahydro-1H-pyrazolo[4,3-c]pyridin-3-yl)-1,2,3,4-tetrahydroquinolin-6-yl)-1H-pyrazol-1-yl)acetic acid (crude product from above reaction) in DCM (10 ml) were added 2,5-dioxopyrrolidin-1-yl methylcarbamate (91.9 mg, 0.528 mmol) and TEA (53.5 mg, 0.528 mmol). The resulting mixture was stirred at room temperature for 5 h, then the reaction mixture was purified by reverse phase flash chromatography to give desired product (81 mg, yield: 81%) as a white solid. MS (ESI) m/z: 570.4 [M+H]⁺.

Procedures for the Synthesis of P300 PROTACs.

### Example 86. 3-(7-(Difluoromethyl)-6-(1-methyl-1H-pyrazol-4-yl)-3,4-dihydroquinolin-1(2H)-yl)-1-(1-((2-(2,6-dioxopiperidin-3-yl)-1,3-dioxoisoindolin-5-yl)glycyl)piperidin-4-yl)-N-methyl-1,4,6,7-tetrahydro-5H-pyrazolo[4,3-c]pyridine-5-carboxamide (P-001)

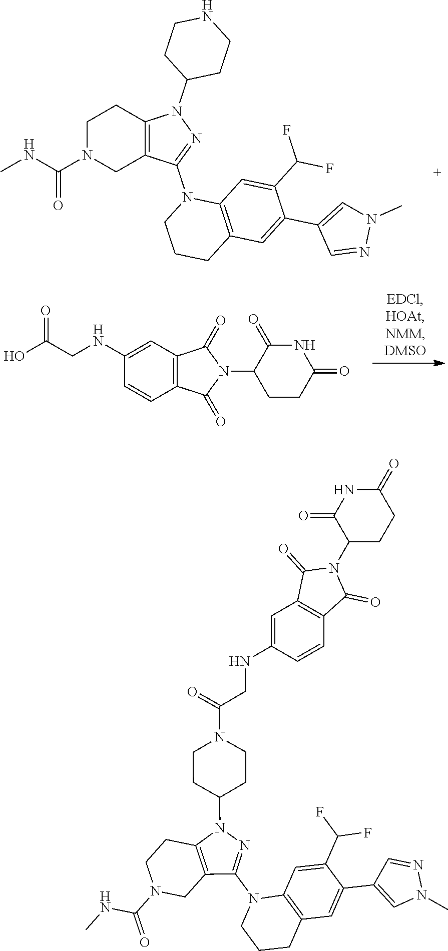

A solution of 3-(7-(difluoromethyl)-6-(1-methyl-1H-pyrazol-4-yl)-3,4-dihydroquinolin-1(2H)-yl)-N-methyl-1-(piperidin-4-yl)-1,4,6,7-tetrahydro-5H-pyrazolo[4,3-c]pyridine-5-carboxamide (8 mg, 0.015 mmol), (2-(2,6-dioxopiperidin-3-yl)-1,3-dioxoisoindolin-5-yl)glycine (6.0 mg, 0.018 mmol), EDCI (4.65 mg, 0.024 mmol), HOAt (3.27 mg, 0.024 mmol) and NMM (9.8 mg, 0.094 mmol) in DMSO (2 mL) was stirred at room temperature overnight. The reaction solution was diluted with EA (20 ml), washed with water (20 ml). The aqueous layer was extracted with EtOAc (20 ml). The combined organic layer was washed with brine twice, dried over Na₂SO₄, filtered and concentrated. The residue was purified with reverse phase flash chromatography to give 3-(7-(difluoromethyl)-6-(1-methyl-1H-pyrazol-4-yl)-3,4-dihydroquinolin-1(2H)-yl)-1-(1-((2-(2,6-dioxopiperidin-3-yl)-1,3-dioxoisoindolin-5-yl)glycyl)piperidin-4-yl)-N-methyl-1,4,6,7-tetrahydro-5H-pyrazolo[4,3-c]pyridine-5-carboxamide (5.5 mg, 44% yield) as a white solid. MS (ESI) m/z: 839.0 [M+H]⁺.

### Example 87. 3-(7-(Difluoromethyl)-6-(1-methyl-1H-pyrazol-4-yl)-3,4-dihydroquinolin-1(2H)-yl)-1-(1-(3-((2-(2,6-dioxopiperidin-3-yl)-1,3-dioxoisoindolin-5-yl)amino)propanoyl)piperidin-4-yl)-N-methyl-1,4,6,7-tetrahydro-5H-pyrazolo[4,3-c]pyridine-5-carboxamide (P-002)

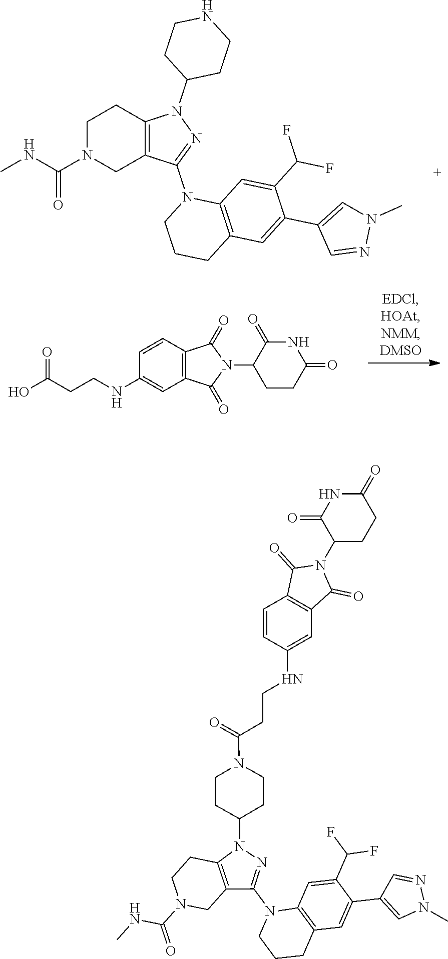

P-002 was synthesized following the standard procedure for preparing P-001 (5.2 mg, yield 41%). MS (ESI) m/z: 853.0 [M+H]⁺.

### Example 88. 3-(7-(Difluoromethyl)-6-(1-methyl-1H-pyrazol-4-yl)-3,4-dihydroquinolin-1(2H)-yl)-1-(1-(4-((2-(2,6-dioxopiperidin-3-yl)-1,3-dioxoisoindolin-5-yl)amino)butanoyl)piperidin-4-yl)-N-methyl-1,4,6,7-tetrahydro-5H-pyrazolo[4,3-c]pyridine-5-carboxamide (P-003)

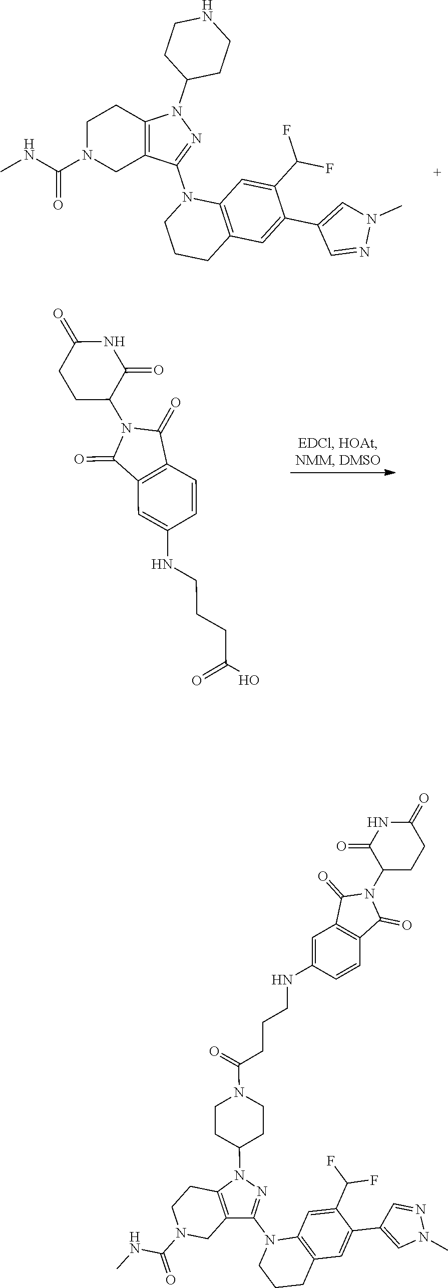

P-003 was synthesized following the standard procedure for preparing P-001 (4.7 mg, yield 36%). MS (ESI) m/z: 867.0 [M+H]⁺.

### Example 89. 3-(7-(Difluoromethyl)-6-(1-methyl-1H-pyrazol-4-yl)-3,4-dihydroquinolin-1(2H)-yl)-1-(1-(5-((2-(2,6-dioxopiperidin-3-yl)-1,3-dioxoisoindolin-5-yl)amino)pentanoyl)piperidin-4-yl)-N-methyl-1,4,6,7-tetrahydro-5H-pyrazolo[4,3-c]pyridine-5-carboxamide (P-004)

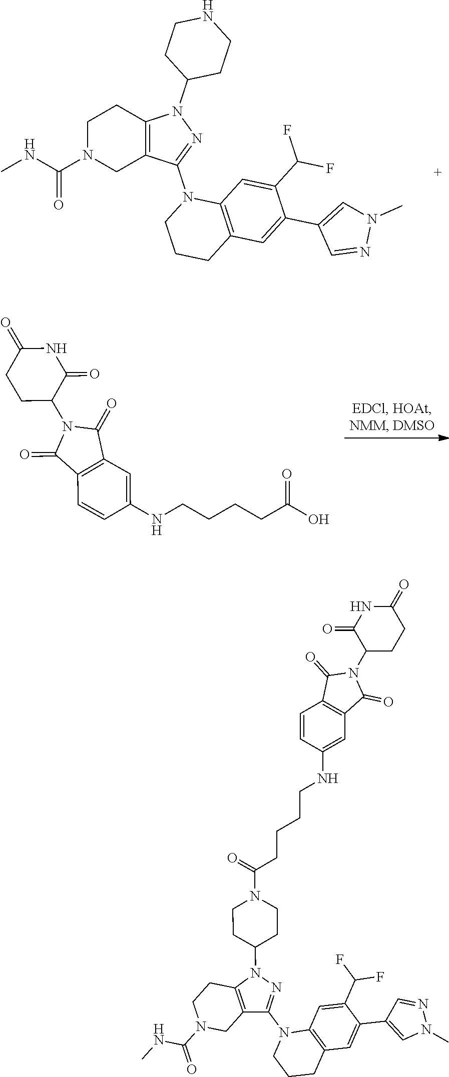

P-004 was synthesized following the standard procedure for preparing P-001 (5.0 mg, yield 38%). MS (ESI) m/z: 881.0 [M+H]⁺.

### Example 90. 3-(7-(Difluoromethyl)-6-(1-methyl-1H-pyrazol-4-yl)-3,4-dihydroquinolin-1(2H)-yl)-1-(1-(6-((2-(2,6-dioxopiperidin-3-yl)-1,3-dioxoisoindolin-5-yl)amino)hexanoyl)piperidin-4-yl)-N-methyl-1,4,6,7-tetrahydro-5H-pyrazolo[4,3-c]pyridine-5-carboxamide (P-005)

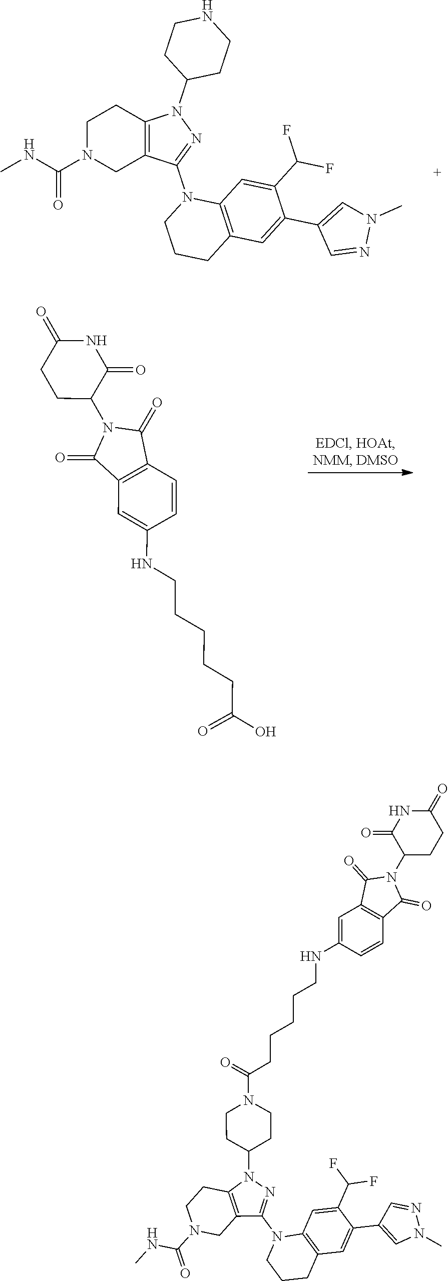

P-005 was synthesized following the standard procedure for preparing P-001 (2.6 mg, yield 16%). MS (ESI) m/z: 895.2 [M+H]⁺.

### Example 91. 3-(7-(Difluoromethyl)-6-(1-methyl-1H-pyrazol-4-yl)-3,4-dihydroquinolin-1(2H)-yl)-1-(1-(7-((2-(2,6-dioxopiperidin-3-yl)-1,3-dioxoisoindolin-5-yl)amino)heptanoyl)piperidin-4-yl)-N-methyl-1,4,6,7-tetrahydro-5H-pyrazolo[4,3-c]pyridine-5-carboxamide (P-006)

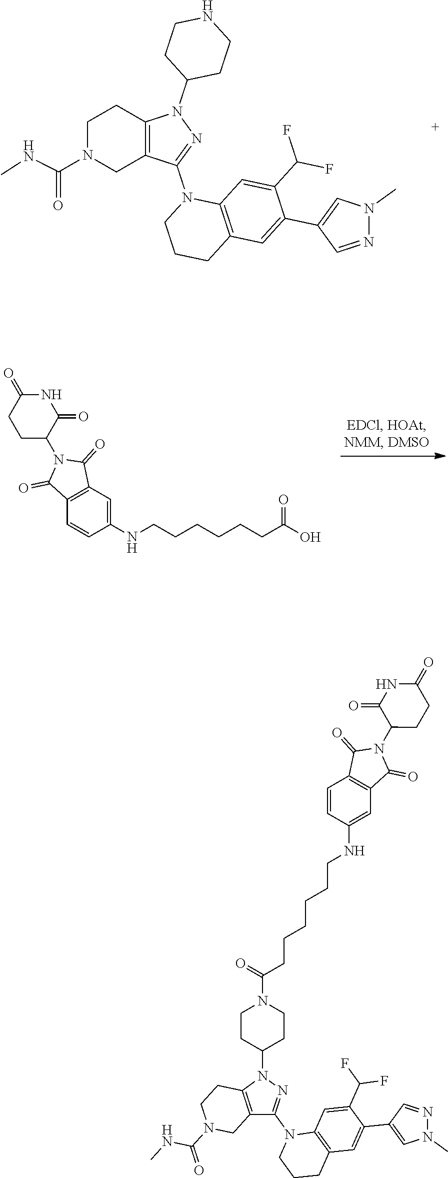

P-006 was synthesized following the standard procedure for preparing P-001 (3.7 mg, yield 27%). MS (ESI) m/z: 909.1 [M+H]⁺.

### Example 92. 3-(7-(Difluoromethyl)-6-(1-methyl-1H-pyrazol-4-yl)-3,4-dihydroquinolin-1(2H)-yl)-1-(1-(8-((2-(2,6-dioxopiperidin-3-yl)-1,3-dioxoisoindolin-5-yl)amino)octanoyl)piperidin-4-yl)-N-methyl-1,4,6,7-tetrahydro-5H-pyrazolo[4,3-c]pyridine-5-carboxamide (P-007)

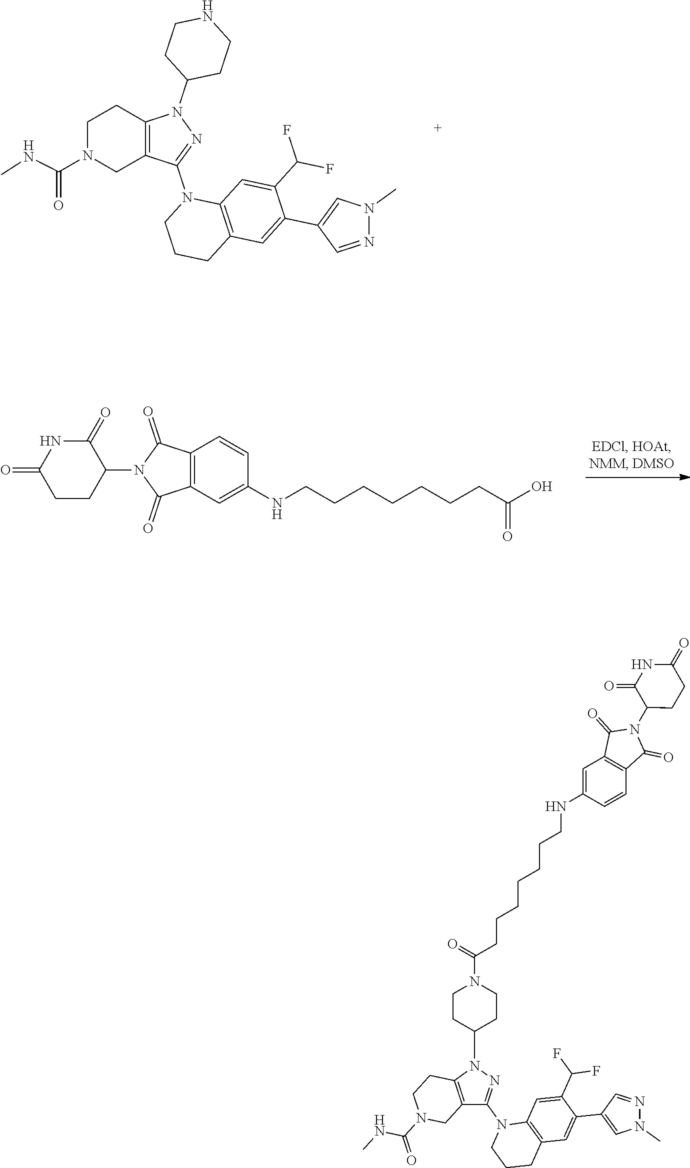

P-007 was synthesized following the standard procedure for preparing P-001 (2.3 mg, yield 17%). MS (ESI) m/z: 923.0 [M+H]⁺.

### Example 93. 3-(7-(Difluoromethyl)-6-(1-methyl-1H-pyrazol-4-yl)-3,4-dihydroquinolin-1(2H)-yl)-1-(1-(3-(2-((2-(2,6-dioxopiperidin-3-yl)-1,3-dioxoisoindolin-5-yl)amino)ethoxy)propanoyl)piperidin-4-yl)-N-methyl-1,4,6,7-tetrahydro-5H-pyrazolo[4,3-c]pyridine-5-carboxamide (P-008)

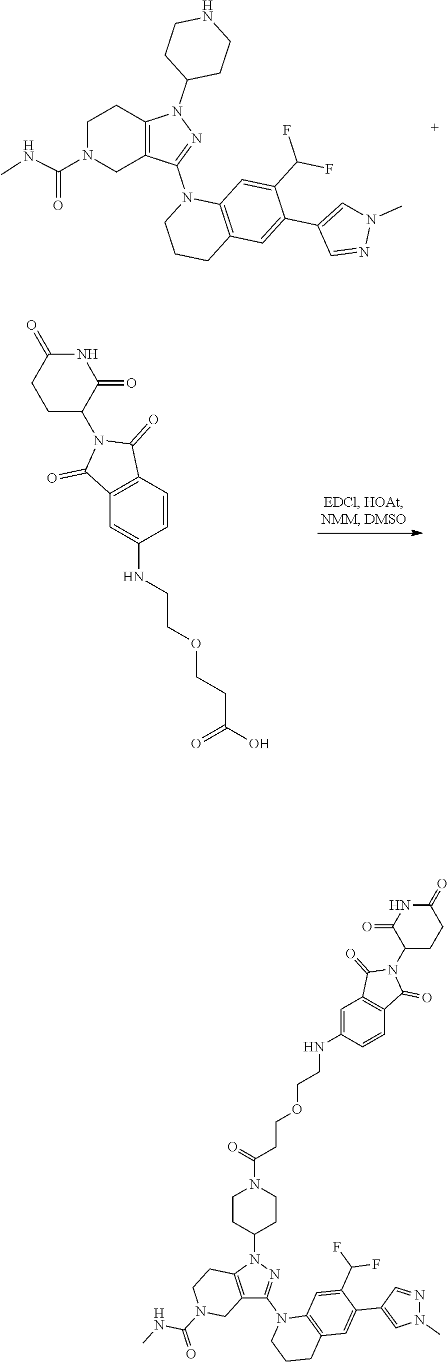

P-008 was synthesized following the standard procedure for preparing P-001 (2.5 mg, yield 19%). MS (ESI) m/z: 897.0 [M+H]⁺.

### Example 94. 3-(7-(difluoromethyl)-6-(1-methyl-1H-pyrazol-4-yl)-3,4-dihydroquinolin-1(2H)-yl)-1-(1-(3-(2-(2-((2-(2,6-dioxopiperidin-3-yl)-1,3-dioxoisoindolin-5-yl)amino)ethoxy)ethoxy)propanoyl)piperidin-4-yl)-N-methyl-1,4,6,7-tetrahydro-5H-pyrazolo[4,3-c]pyridine-5-carboxamide (P-009)

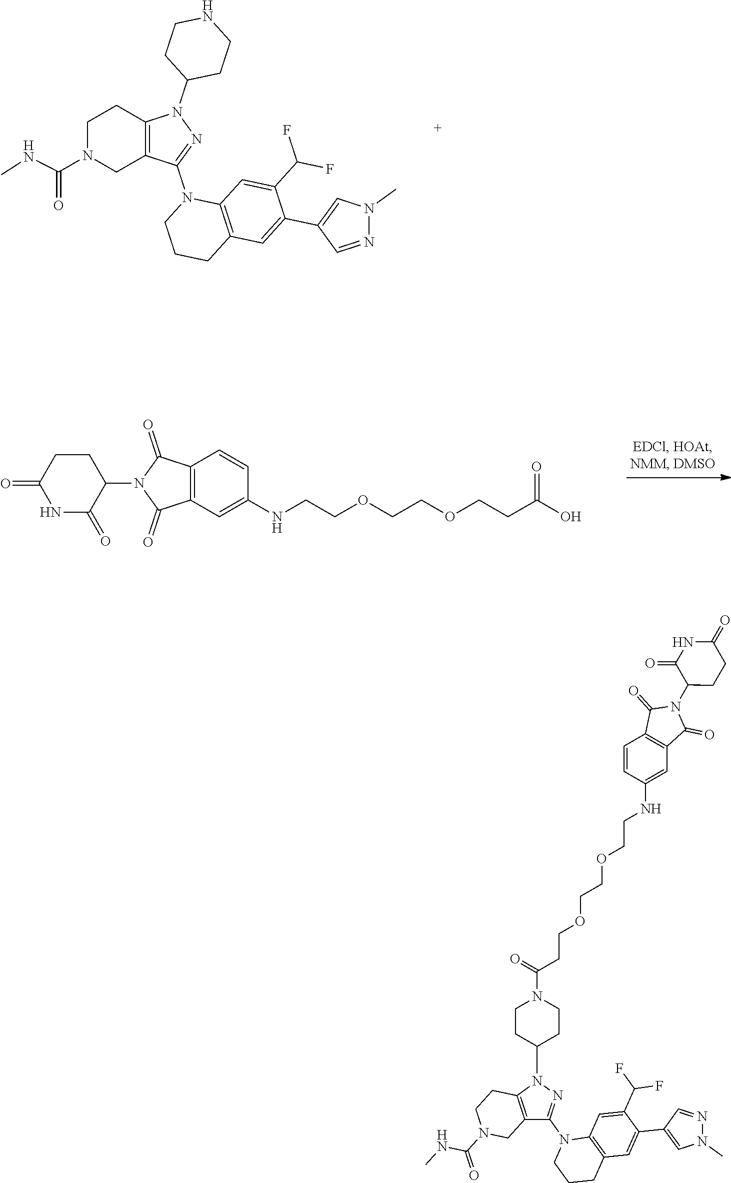

P-009 was synthesized following the standard procedure for preparing P-001 (2.5 mg, yield 18%). MS (ESI) m/z: 941.0 [M+H]⁺.

### Example 95. 3-(7-(Difluoromethyl)-6-(1-methyl-1H-pyrazol-4-yl)-3,4-dihydroquinolin-1(2H)-yl)-1-(1-(3-(2-(2-(2-((2-(2,6-dioxopiperidin-3-yl)-1,3-dioxoisoindolin-5-yl)amino)ethoxy)ethoxy)ethoxy)propanoyl)piperidin-4-yl)-N-methyl-1,4,6,7-tetrahydro-5H-pyrazolo[4,3-c]pyridine-5-carboxamide (P-010)

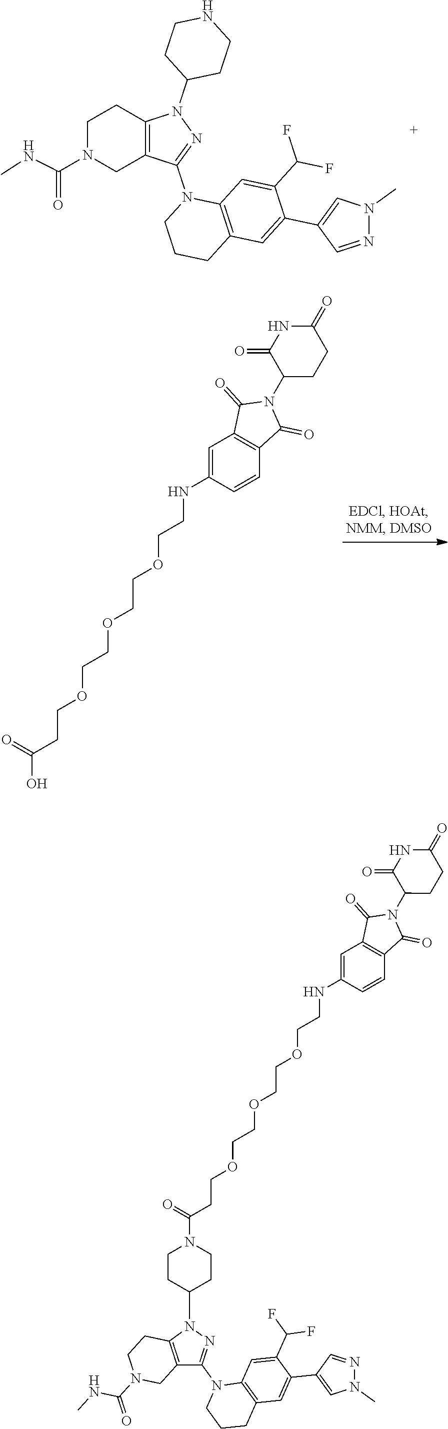

P-010 was synthesized following the standard procedure for preparing P-001 (2.5 mg, yield 17%). MS (ESI) m/z: 985.1 [M+H]⁺.

### Example 96. 3-(7-(Difluoromethyl)-6-(1-methyl-1H-pyrazol-4-yl)-3,4-dihydroquinolin-1(2H)-yl)-1-(1-(1-((2-(2,6-dioxopiperidin-3-yl)-1,3-dioxoisoindolin-5-yl)amino)-3,6,9,12-tetraoxapentadecan-15-oyl)piperidin-4-yl)-N-methyl-1,4,6,7-tetrahydro-5H-pyrazolo[4,3-c]pyridine-5-carboxamide (P-011)

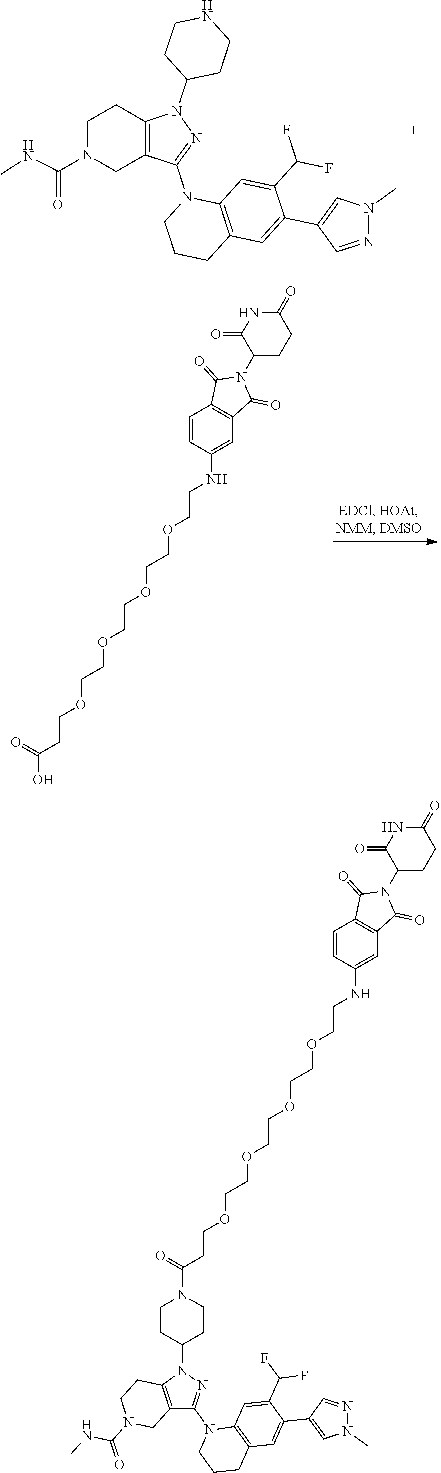

P-011 was synthesized following the standard procedure for preparing P-001 (2.6 mg, yield 17%). MS (ESI) m/z: 1027.1 [M+H]⁺.

### Example 97. 3-(7-(Difluoromethyl)-6-(1-methyl-1H-pyrazol-4-yl)-3,4-dihydroquinolin-1(2H)-yl)-1-(1-(1-((2-(2,6-dioxopiperidin-3-yl)-1,3-dioxoisoindolin-5-yl)amino)-3,6,9,12,15-pentaoxaoctadecan-18-oyl)piperidin-4-yl)-N-methyl-1,4,6,7-tetrahydro-5H-pyrazolo[4,3-c]pyridine-5-carboxamide (P-012)

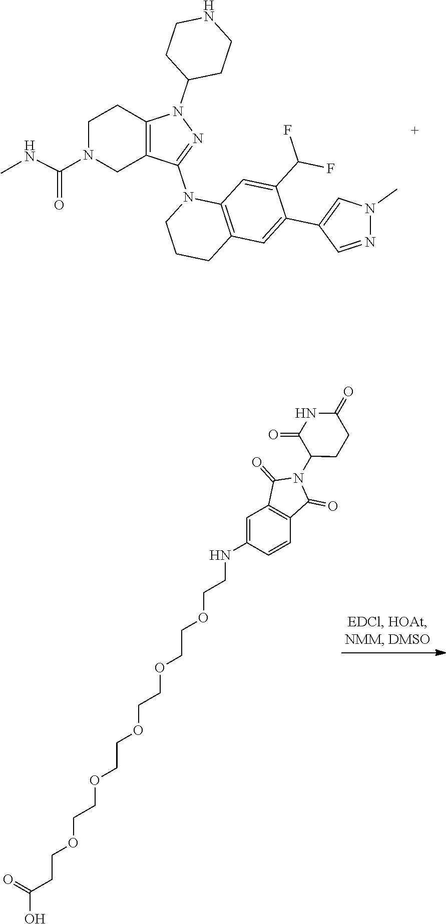

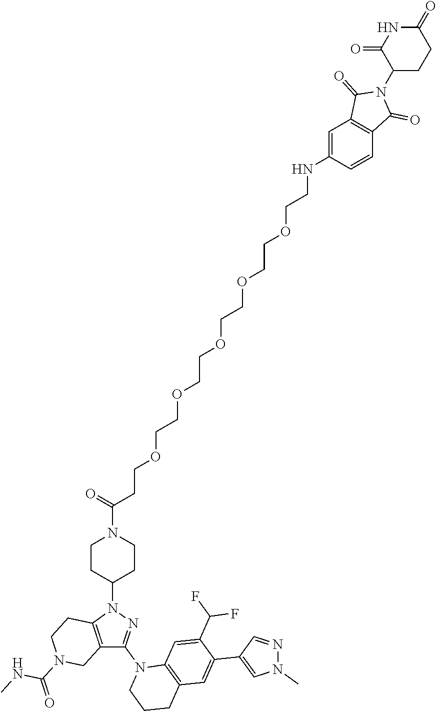 [SMILES: CNC(=O)N1CCc2c(c(N3CCCc4cc(-c5cnn(C)c5)c(C(F)F)cc43)nn2C2CCN(C(=O)CCOCCOCCOCCOCCOCCNc3ccc4c(c3)C(=O)N(C3CCC(=O)NC3=O)C4=O)CC2)C1]

P-012 was synthesized following the standard procedure for preparing P-001 (3.5 mg, yield 22%). MS (ESI) m/z: 1073.2 [M+H]⁺.

### Example 98. N-(4-(3-(2,6-Difluoro-3-(propylsulfonamido)benzoyl)-1H-pyrrolo[2,3-b]pyridin-5-yl)benzyl)-8-((2-(2,6-dioxopiperidin-3-yl)-1,3-dioxoisoindolin-4-yl)amino)octanamide (P-013)

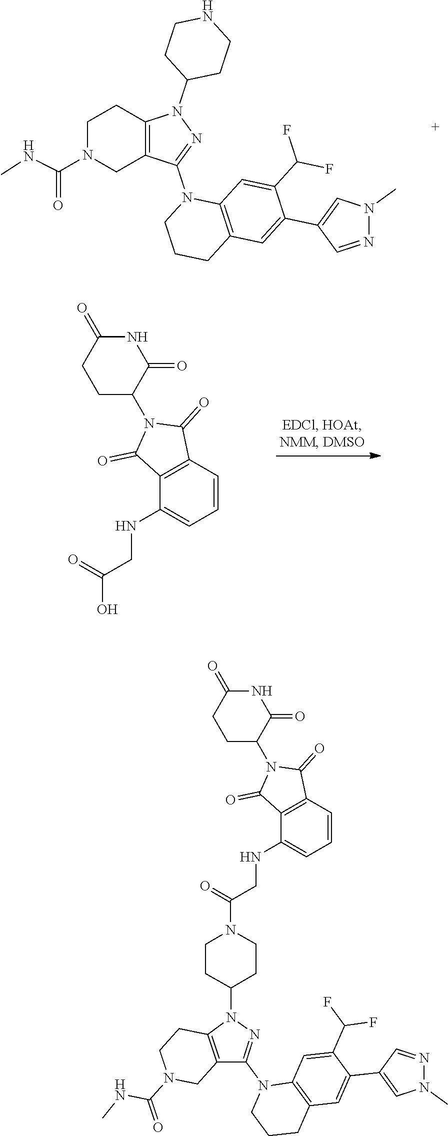

P-013 was synthesized following the standard procedure for preparing P-001 (3.2 mg, yield 25%). MS (ESI) m/z: 838.9 [M+H]⁺.

### Example 99. 3-(7-(Difluoromethyl)-6-(1-methyl-1H-pyrazol-4-yl)-3,4-dihydroquinolin-1(2H)-yl)-1-(1-(3-((2-(2,6-dioxopiperidin-3-yl)-1,3-dioxoisoindolin-4-yl)amino)propanoyl)piperidin-4-yl)-N-methyl-1,4,6,7-tetrahydro-5H-pyrazolo[4,3-c]pyridine-5-carboxamide (P-014)

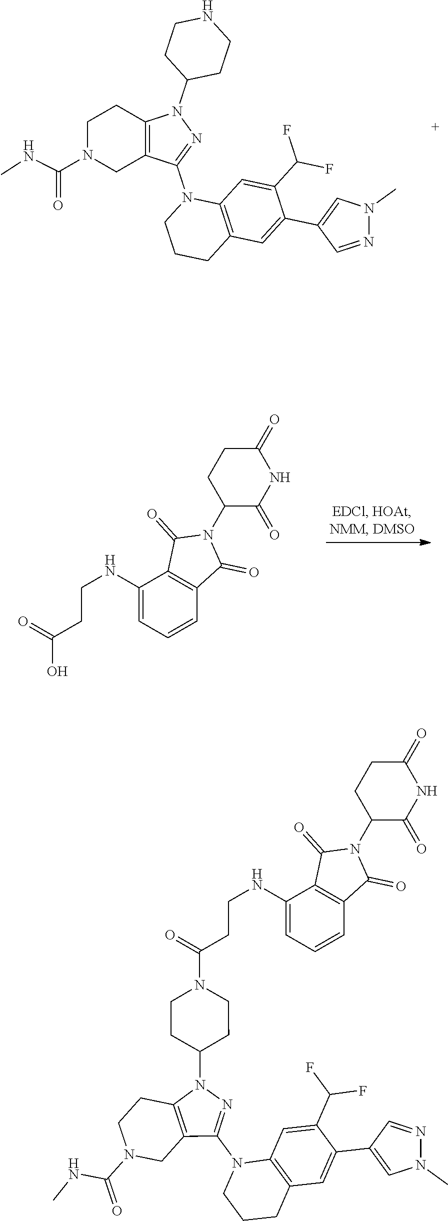

P-014 was synthesized following the standard procedure for preparing P-001 (2.6 mg, yield 20%). MS (ESI) m/z: 853.0 [M+H]⁺.

### Example 100. 3-(7-(Difluoromethyl)-6-(1-methyl-1H-pyrazol-4-yl)-3,4-dihydroquinolin-1(2H)-yl)-1-(1-(4-((2-(2,6-dioxopiperidin-3-yl)-1,3-dioxoisoindolin-4-yl)amino)butanoyl)piperidin-4-yl)-N-methyl-1,4,6,7-tetrahydro-5H-pyrazolo[4,3-c]pyridine-5-carboxamide (P-015)

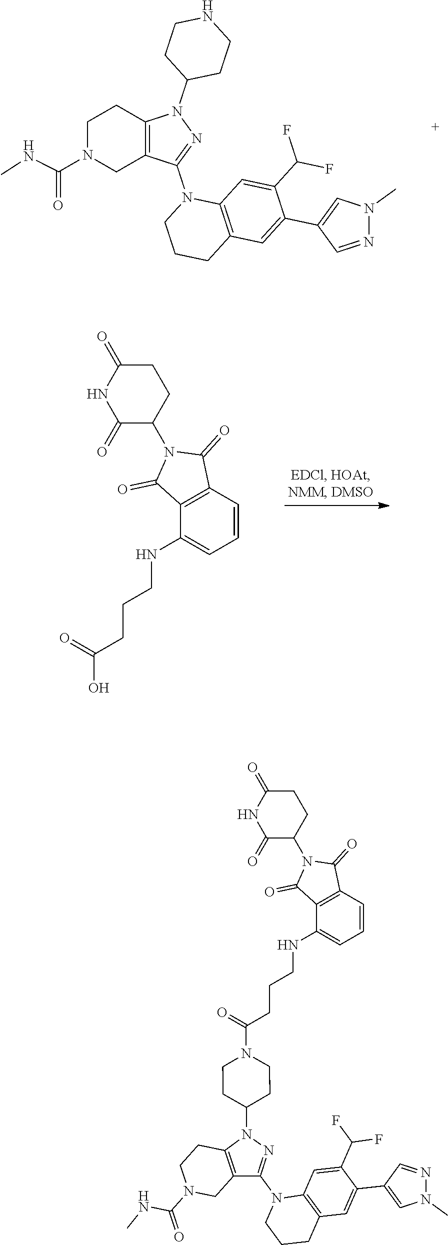

P-015 was synthesized following the standard procedure for preparing P-001 (2.5 mg, yield 19%). MS (ESI) m/z: 867.1 [M+H]⁺.

### Example 101. 3-(7-(Difluoromethyl)-6-(1-(2-((2-((2-(2,6-dioxopiperidin-3-yl)-1,3-dioxoisoindolin-5-yl)amino)ethyl)amino)-2-oxoethyl)-1H-pyrazol-4-yl)-3,4-dihydroquinolin-1(2H)-yl)-N-methyl-1-(tetrahydro-2H-pyran-4-yl)-1,4,6,7-tetrahydro-5H-pyrazolo[4,3-c]pyridine-5-carboxamide (P-016)

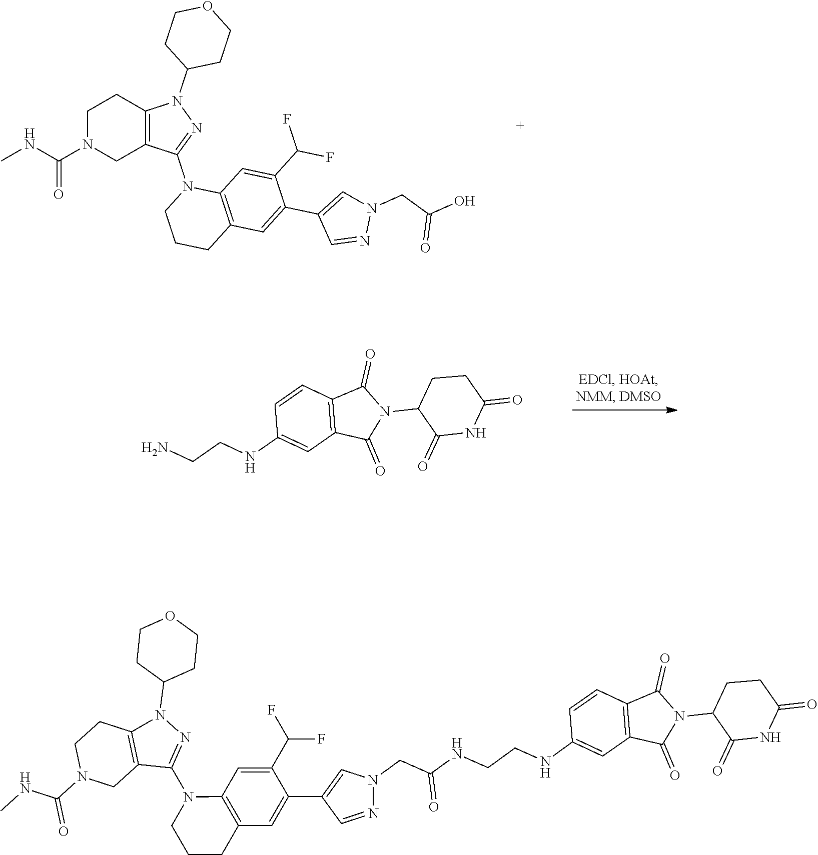

P-016 was synthesized following the standard procedure for preparing P-001 (4.8 mg, yield 37%). MS (ESI) m/z: 869.2 [M+H]⁺.

### Example 102. 3-(7-(Difluoromethyl)-6-(1-(2-((3-((2-(2,6-dioxopiperidin-3-yl)-1,3-dioxoisoindolin-5-yl)amino)propyl)amino)-2-oxoethyl)-1H-pyrazol-4-yl)-3,4-dihydroquinolin-1(2H)-yl)-N-methyl-1-(tetrahydro-2H-pyran-4-yl)-1,4,6,7-tetrahydro-5H-pyrazolo[4,3-c]pyridine-5-carboxamide (P-017)

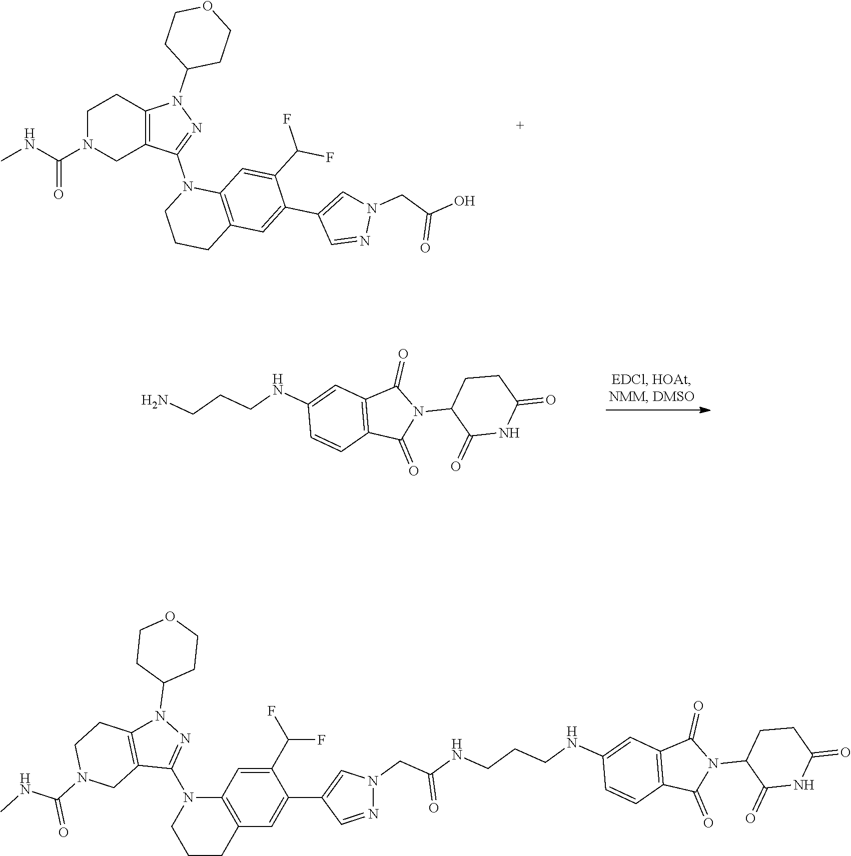

P-017 was synthesized following the standard procedure for preparing P-001 (4.7 mg, 35%). MS (ESI) m/z: 883.0 [M+H]⁺.

### Example 103. N1-(4-(3-(2,6-difluoro-3-(propylsulfonamido)benzoyl)-1H-pyrrolo[2,3-b] pyridin-5-yl)benzyl)-N5-(S)-1-((2S,4R)-4-hydroxy-2-((4-(4-methylthiazol-5-yl)benzyl)carbamoyl)pyrrolidin-1-yl)-3,3-dimethyl-1-oxobutan-2-yl)glutaramide (P-018)

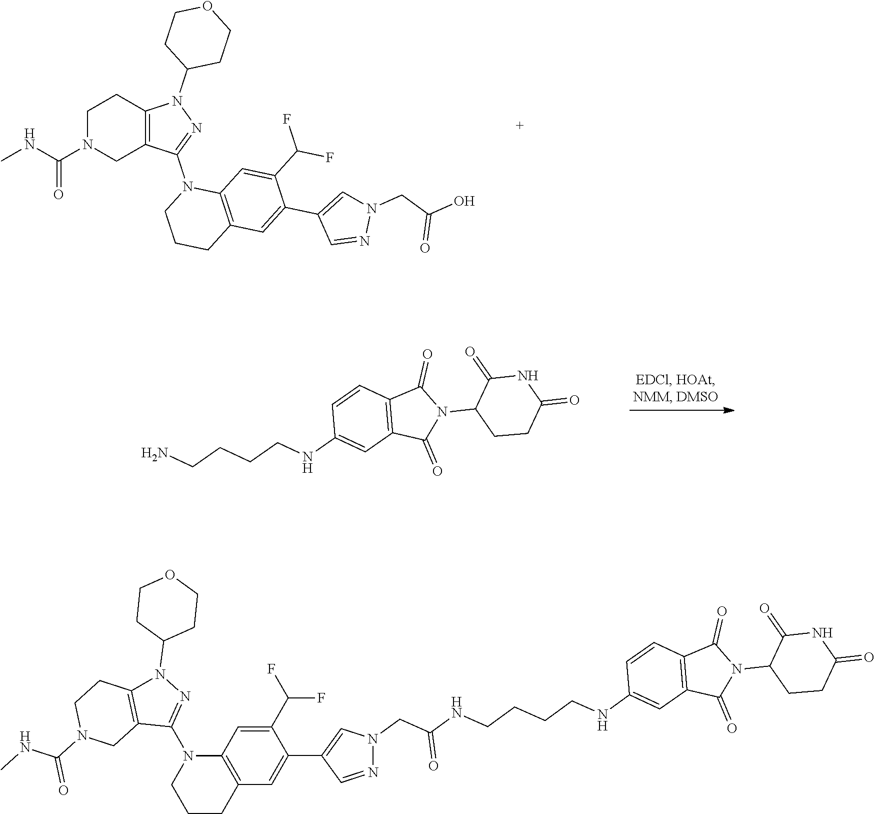

P-018 was synthesized following the standard procedure for preparing P-001 (6.0 mg, yield 45%). MS (ESI) m/z: 897.0 [M+H]⁺.

### Example 104. 3-(7-(Difluoromethyl)-6-(1-(24(5-((2-(2,6-dioxopiperidin-3-yl)-1,3-dioxoisoindolin-5-yl)amino)pentyl)amino)-2-oxoethyl)-1H-pyrazol-4-yl)-3,4-dihydroquinolin-1(2H)-yl)-N-methyl-1-(tetrahydro-2H-pyran-4-yl)-1,4,6,7-tetrahydro-5H-pyrazolo[4,3-c]pyridine-5-carboxamide (P-019)

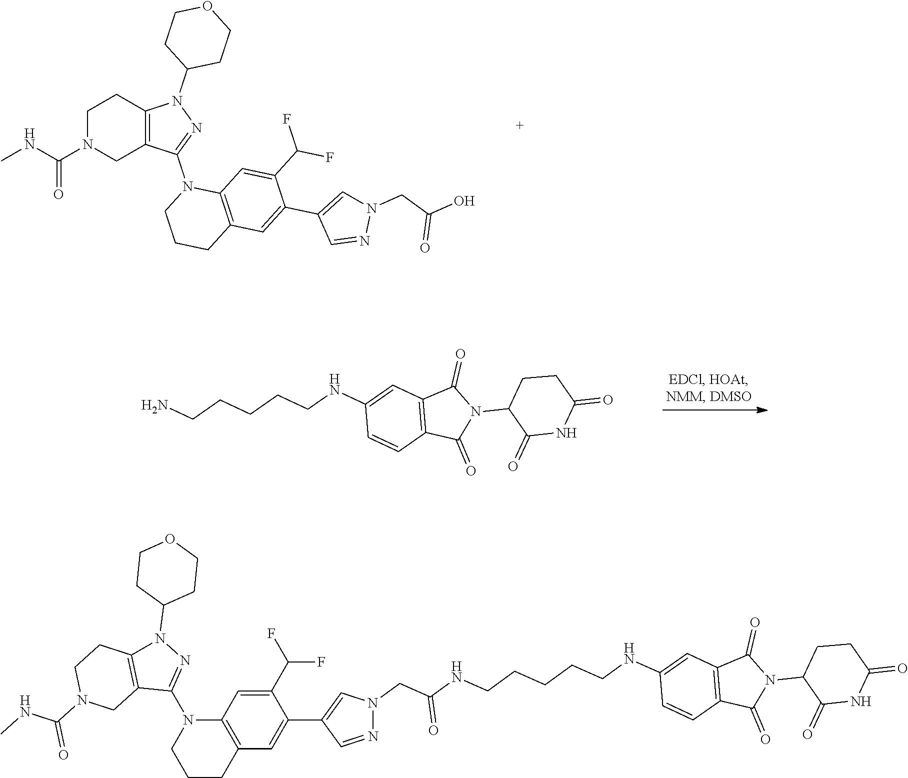

P-019 was synthesized following the standard procedure for preparing P-001 (2.6 mg, yield 19%). MS (ESI) m/z: 911.1 [M+H]⁺.

### Example 105. 3-(7-(Difluoromethyl)-6-(1-(2-((6-((2-(2,6-dioxopiperidin-3-yl)-1,3-dioxoisoindolin-5-yl)amino)hexyl)amino)-2-oxoethyl)-1H-pyrazol-4-yl)-3,4-dihydroquinolin-1(2H)-yl)-N-methyl-1-(tetrahydro-2H-pyran-4-yl)-1,4,6,7-tetrahydro-5H-pyrazolo[4,3-c]pyridine-5-carboxamide (P-020)

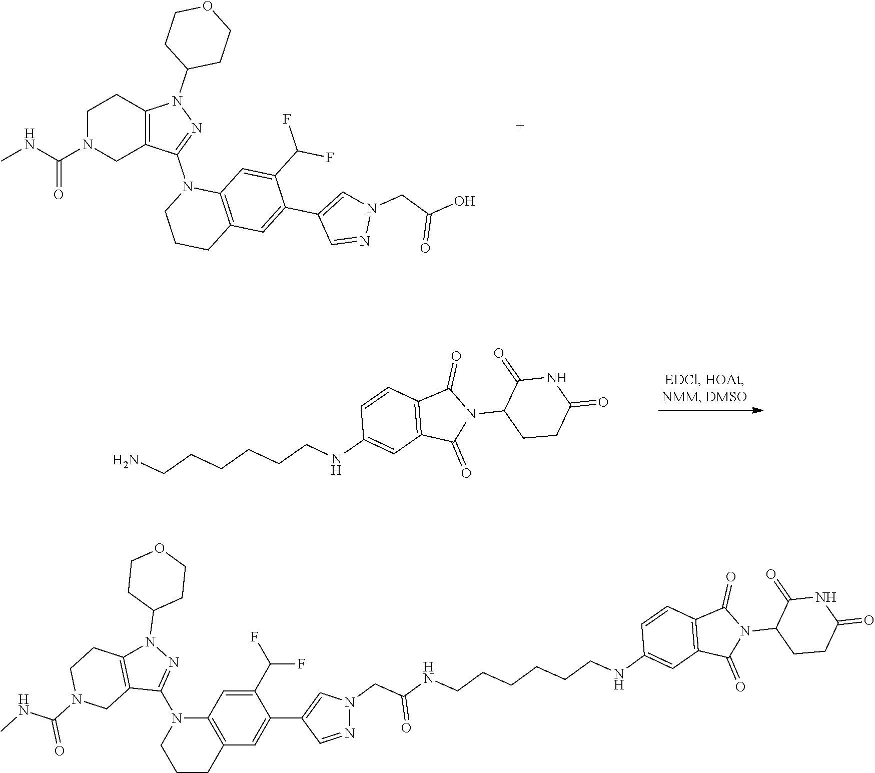

P-020 was synthesized following the standard procedure for preparing P-001 (5.0 mg, yield 36%). MS (ESI) m/z: 925.1 [M+H]⁺.

### Example 106. (2S,4R)-1-((S)-2-(2-(2-((4-(3-(2,6-difluoro-3-(propylsulfonamido)benzoyl)-1H-pyrrolo[2,3-b]pyridin-5-yl)benzyl)amino)-2-oxoethoxy)acetamido)-3,3-dimethylbutanoyl)-4-hydroxy-N-(4-(4-methylthiazol-5-yl)benzyl)pyrrolidine-2-carboxamide (P-021)

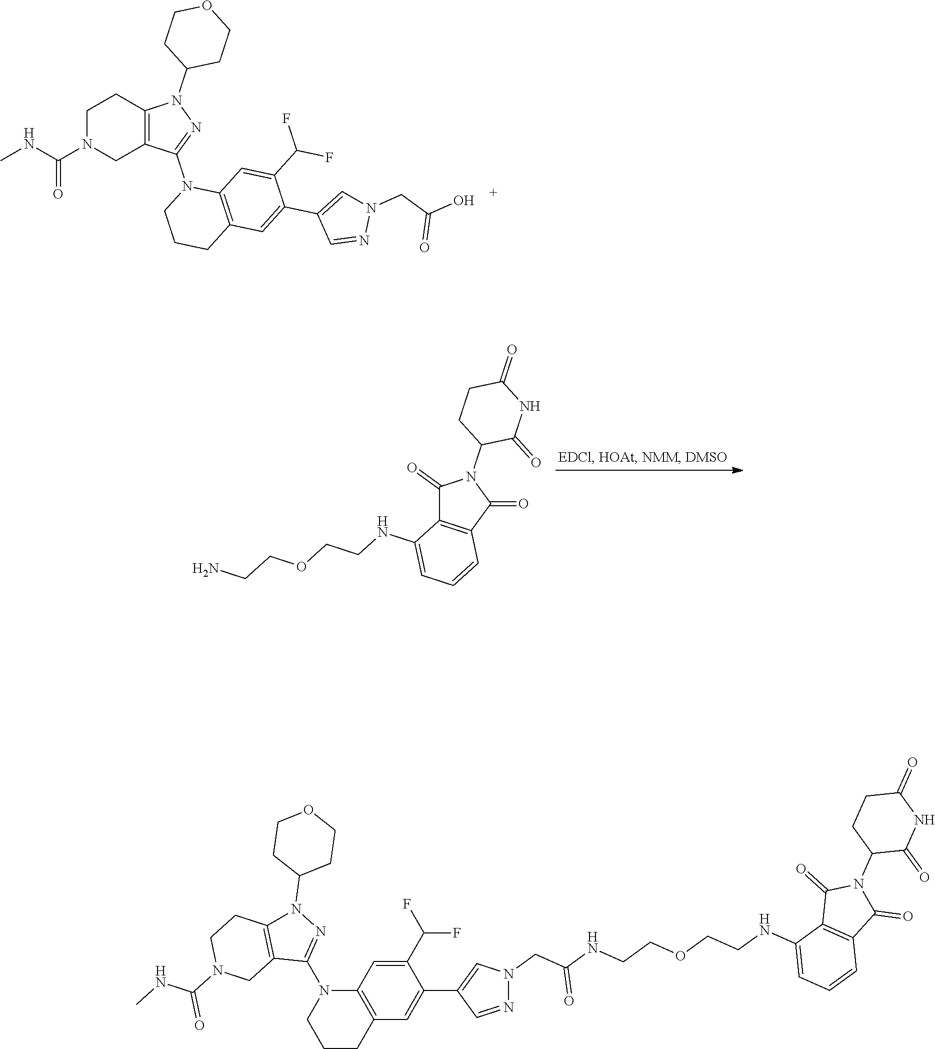 [SMILES: CNC(=O)N1CCc2c(c(N3CCCc4cc(-c5cnn(CC(=O)O)c5)c(C(F)F)cc43)nn2C2CCOCC2)C1 ; NCCOCCNc1cccc2c1C(=O)N(C1CCC(=O)NC1=O)C2=O ; CNC(=O)N1CCc2c(c(N3CCCc4cc(-c5cnn(CC(=O)NCCOCCNc6cccc7c6C(=O)N(C6CCC(=O)NC6=O)C7=O)c5)c(C(F)F)cc43)nn2C2CCOCC2)C1]

P-021 was synthesized following the standard procedure for preparing P-001 (5.3 mg, yield 39%). MS (ESI) m/z: 913.0 [M+H]⁺.

### Example 107. 3-(7-(Difluoromethyl)-6-(1-(2-((2-(2-(2-((2-(2,6-dioxopiperidin-3-yl)-1,3-dioxoisoindolin-4-yl)amino)ethoxy)ethoxy)ethyl)amino)-2-oxoethyl)-1H-pyrazol-4-yl)-3,4-dihydroquinolin-1(2H)-yl)-N-methyl-1-(tetrahydro-2H-pyran-4-yl)-1,4,6,7-tetrahydro-5H-pyrazolo[4,3-c]pyridine-5-carboxamide (P-022)

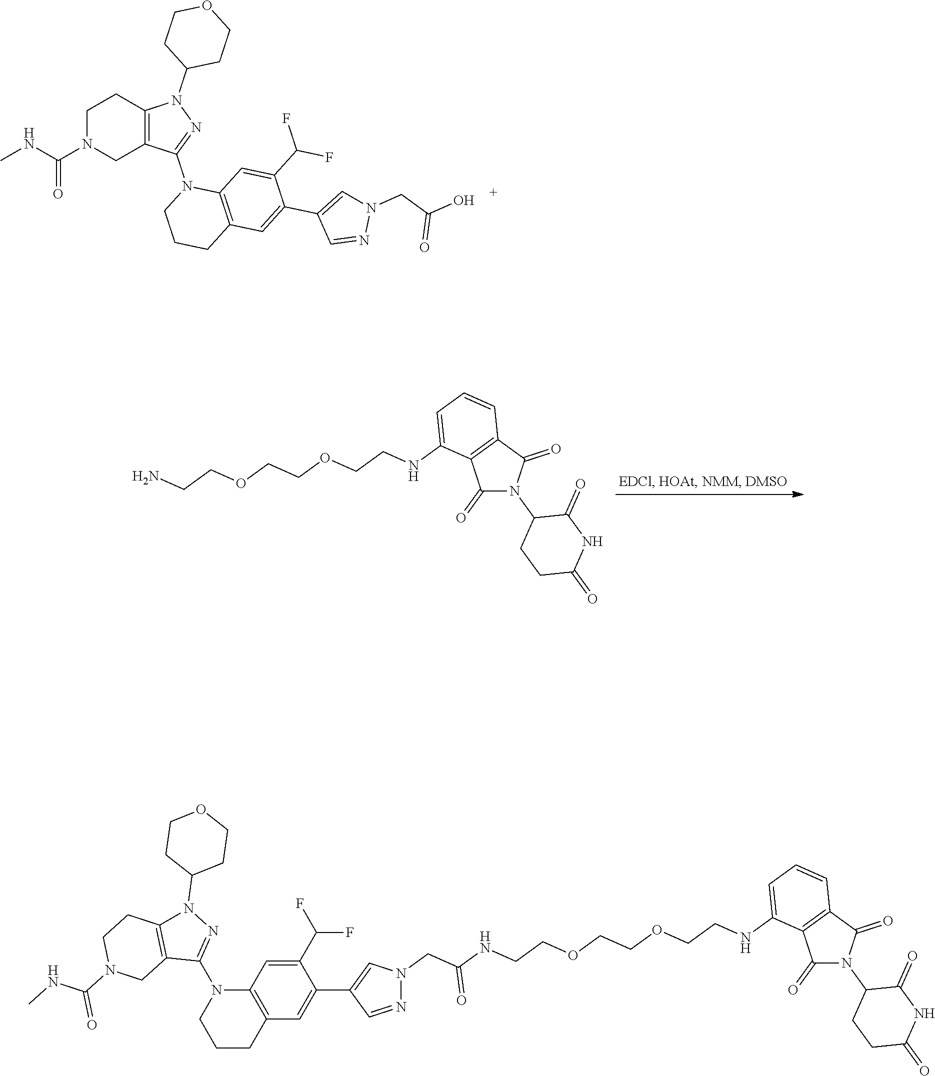 [SMILES: CNC(=O)N1CCc2c(c(N3CCCc4cc(-c5cnn(CC(=O)O)c5)c(C(F)F)cc43)nn2C2CCOCC2)C1 ; NCCOCCOCCNc1cccc2c1C(=O)N(C1CCC(=O)NC1=O)C2=O ; CNC(=O)N1CCc2c(c(N3CCCc4cc(-c5cnn(CC(=O)NCCOCCOCCNc6cccc7c6C(=O)N(C6CCC(=O)NC6=O)C7=O)c5)c(C(F)F)cc43)nn2C2CCOCC2)C1]

P-022 was synthesized following the standard procedure for preparing P-001 (5.3 mg, yield 37%). MS (ESI) m/z: 957.0 [M+H]⁺.

### Example 108. 3-(7-(Difluoromethyl)-6-(1-(14-((2-(2,6-dioxopiperidin-3-yl)-1,3-dioxoisoindolin-4-yl)amino)-2-oxo-6,9,12-trioxa-3-azatetradecyl)-1H-pyrazol-4-yl)-3,4-dihydroquinolin-1(2H)-yl)-N-methyl-1-(tetrahydro-2H-pyran-4-yl)-1,4,6,7-tetrahydro-5H-pyrazolo[4,3-c]pyridine-5-carboxamide (P-023)

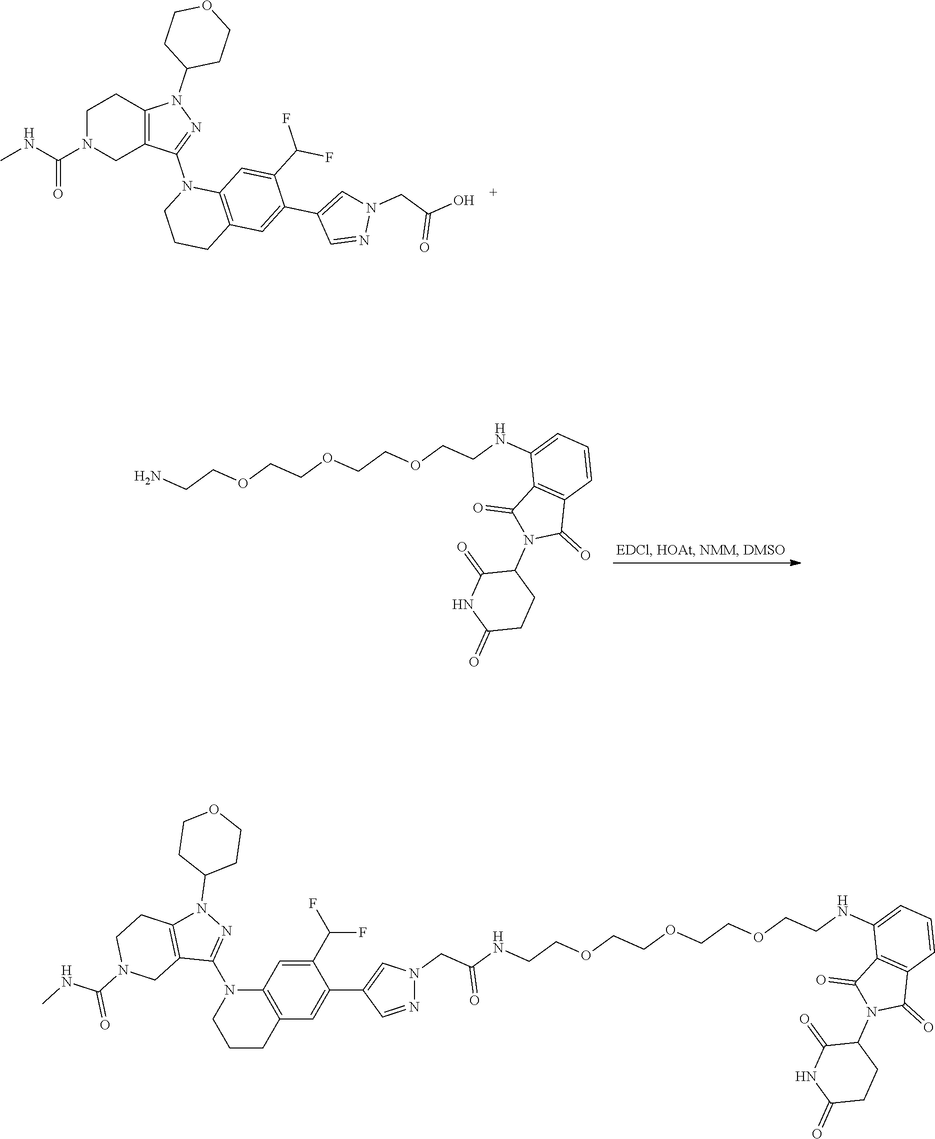 [SMILES: CNC(=O)N1CCc2c(c(N3CCCc4cc(-c5cnn(CC(=O)O)c5)c(C(F)F)cc43)nn2C2CCOCC2)C1 ; NCCOCCOCCOCCNc1cccc2c1C(=O)N(C1CCC(=O)NC1=O)C2=O ; CNC(=O)N1CCc2c(c(N3CCCc4cc(-c5cnn(CC(=O)NCCOCCOCCOCCNc6cccc7c6C(=O)N(C6CCC(=O)NC6=O)C7=O)c5)c(C(F)F)cc43)nn2C2CCOCC2)C1]

P-023 was synthesized following the standard procedure for preparing P-001 (5.5 mg, yield 37%). MS (ESI) m/z: 1000.9 [M+H]⁺.

### Example 109. 3-(7-(Difluoromethyl)-6-(1-(17-((2-(2,6-dioxopiperidin-3-yl)-1,3-dioxoisoindolin-4-yl)amino)-2-oxo-6,9,12,15-tetraoxa-3-azaheptadecyl)-1H-pyrazol-4-yl)-3,4-dihydroquinolin-1(2H)-yl)-N-methyl-1-(tetrahydro-2H-pyran-4-yl)-1,4,6,7-tetrahydro-5H-pyrazolo[4,3-c]pyridine-5-carboxamide (P-024)

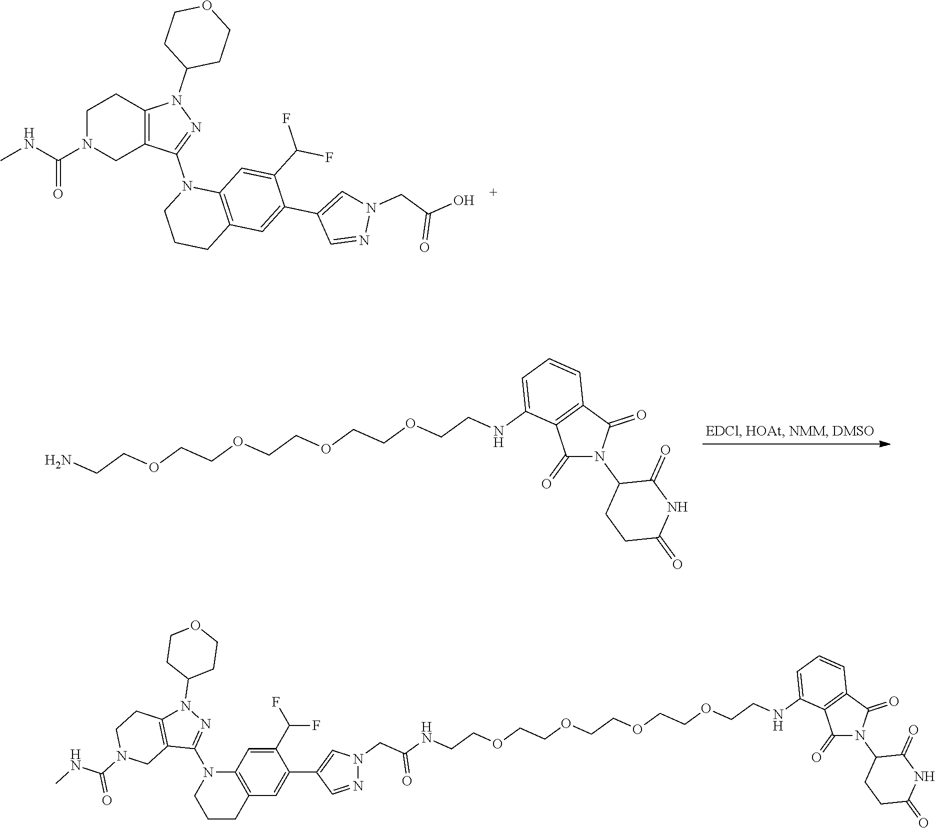 [SMILES: CNC(=O)N1CCc2c(c(N3CCCc4cc(-c5cnn(CC(=O)O)c5)c(C(F)F)cc43)nn2C2CCOCC2)C1 ; NCCOCCOCCOCCOCCNc1cccc2c1C(=O)N(C1CCC(=O)NC1=O)C2=O ; CNC(=O)N1CCc2c(c(N3CCCc4cc(-c5cnn(CC(=O)NCCOCCOCCOCCOCCNc6cccc7c6C(=O)N(C6CCC(=O)NC6=O)C7=O)c5)c(C(F)F)cc43)nn2C2CCOCC2)C1]

P-024 was synthesized following the standard procedure for preparing P-001 (5.4 mg, yield 34%). MS (ESI) m/z: 1045.0 [M+H]⁺.

### Example 110. 3-(7-(Difluoromethyl)-6-(1-(20-((2-(2,6-dioxopiperidin-3-yl)-1,3-dioxoisoindolin-4-yl)amino)-2-oxo-6,9,12,15,18-pentaoxa-3-azaicosyl)-1H-pyrazol-4-yl)-3,4-dihydroquinolin-1(2H)-yl)-N-methyl-1-(tetrahydro-2H-pyran-4-yl)-1,4,6,7-tetrahydro-5H-pyrazolo[4,3-c]pyridine-5-carboxamide (P-025)

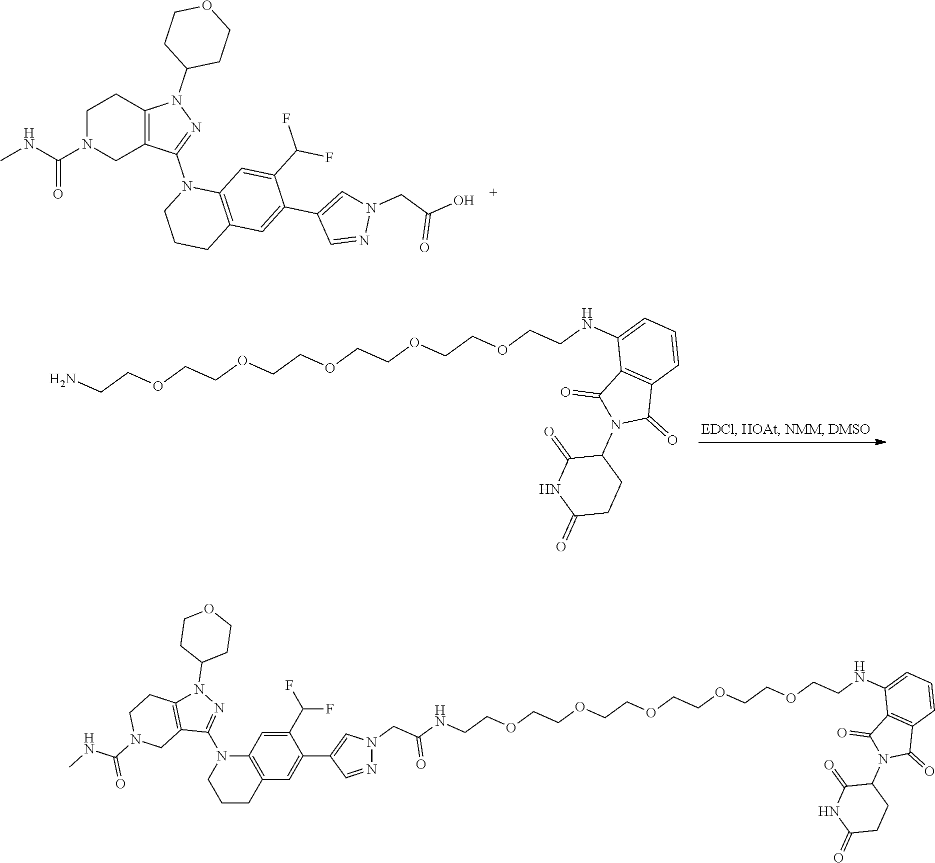 [SMILES: CNC(=O)N1CCc2c(c(N3CCCc4cc(-c5cnn(CC(=O)O)c5)c(C(F)F)cc43)nn2C2CCOCC2)C1 ; NCCOCCOCCOCCOCCOCCNc1cccc2c1C(=O)N(C1CCC(=O)NC1=O)C2=O ; CNC(=O)N1CCc2c(c(N3CCCc4cc(-c5cnn(CC(=O)NCCOCCOCCOCCOCCOCCNc6cccc7c6C(=O)N(C6CCC(=O)NC6=O)C7=O)c5)c(C(F)F)cc43)nn2C2CCOCC2)C1]

P-025 was synthesized following the standard procedure for preparing P-001 (5.5 mg, yield 34%). MS (ESI) m/z: 1089.2 [M+H]⁺.

### Example 111. 3-(7-(Difluoromethyl)-6-(1-(2-((7-((2-(2,6-dioxopiperidin-3-yl)-1,3-dioxoisoindolin-5-yl)amino)heptyl)amino)-2-oxoethyl)-1H-pyrazol-4-yl)-3,4-dihydroquinolin-1(2H)-yl)-N-methyl-1-(tetrahydro-2H-pyran-4-yl)-1,4,6,7-tetrahydro-5H-pyrazolo[4,3-c]pyridine-5-carboxamide (P-026)

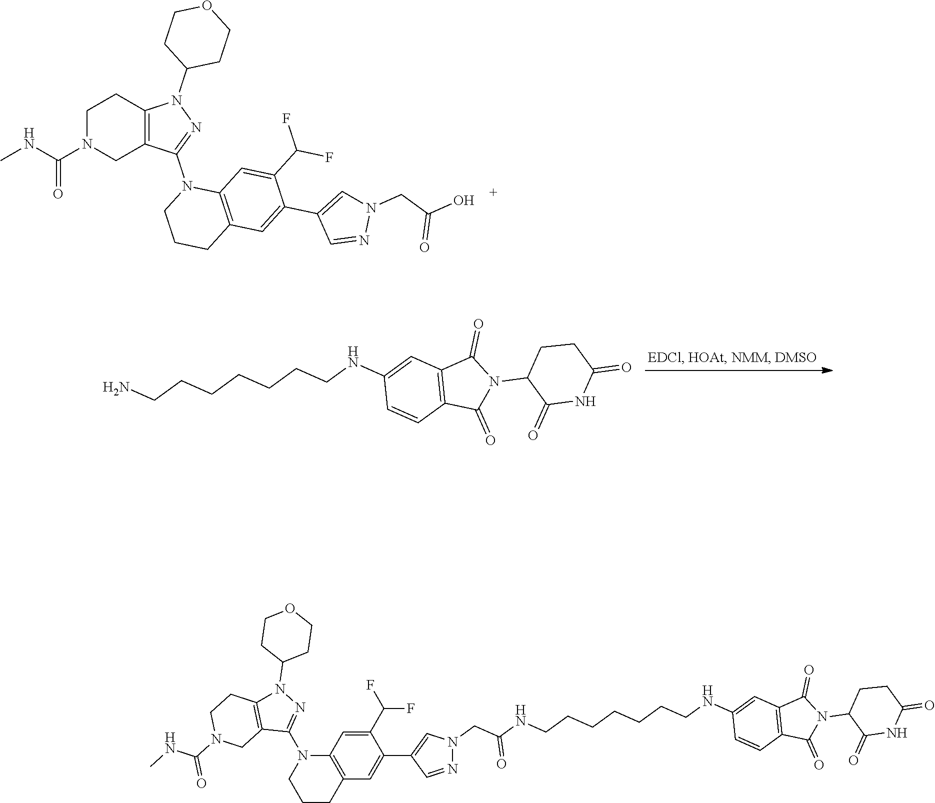 [SMILES: CNC(=O)N1CCc2c(c(N3CCCc4cc(-c5cnn(CC(=O)O)c5)c(C(F)F)cc43)nn2C2CCOCC2)C1 ; NCCCCCCCNc1ccc2c(c1)C(=O)N(C1CCC(=O)NC1=O)C2=O ; CNC(=O)N1CCc2c(c(N3CCCc4cc(-c5cnn(CC(=O)NCCCCCCCNc6ccc7c(c6)C(=O)N(C6CCC(=O)NC6=O)C7=O)c5)c(C(F)F)cc43)nn2C2CCOCC2)C1]

P-026 was synthesized following the standard procedure for preparing P-001 (3.5 mg, yield 25%). MS (ESI) m/z: 938.9 [M+H]⁺.

### Example 112. 3-(7-(Difluoromethyl)-6-(1-(2-((8-((2-(2,6-dioxopiperidin-3-yl)-1,3-dioxoisoindolin-5-yl)amino)octyl)amino)-2-oxoethyl)-1H-pyrazol-4-yl)-3,4-dihydroquinolin-1(2H)-yl)-N-methyl-1-(tetrahydro-2H-pyran-4-yl)-1,4,6,7-tetrahydro-5H-pyrazolo[4,3-c]pyridine-5-carboxamide (P-027)

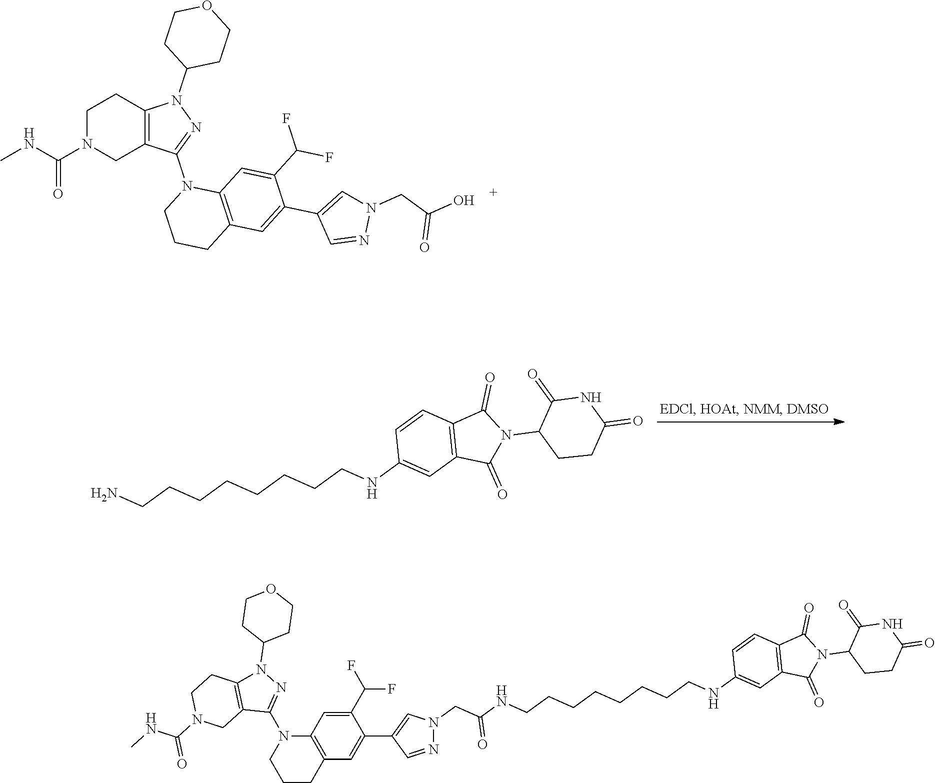 [SMILES: CNC(=O)N1CCc2c(c(N3CCCc4cc(-c5cnn(CC(=O)O)c5)c(C(F)F)cc43)nn2C2CCOCC2)C1 ; NCCCCCCCCNc1ccc2c(c1)C(=O)N(C1CCC(=O)NC1=O)C2=O ; CNC(=O)N1CCc2c(c(N3CCCc4cc(-c5cnn(CC(=O)NCCCCCCCCNc6ccc7c(c6)C(=O)N(C6CCC(=O)NC6=O)C7=O)c5)c(C(F)F)cc43)nn2C2CCOCC2)C1]

P-027 was synthesized following the standard procedure for preparing P-001 (4.7 mg, yield 33%). MS (ESI) m/z: 953.0 [M+H]⁺.

### Example 113. 3-(7-(Difluoromethyl)-6-(1-(2-((2-(2-((2-(2,6-dioxopiperidin-3-yl)-1,3-dioxoisoindolin-5-yl)amino)ethoxy)ethyl)amino)-2-oxoethyl)-1H-pyrazol-4-yl)-3,4-dihydroquinolin-1(2H)-yl)-N-methyl-1-(tetrahydro-2H-pyran-4-yl)-1,4,6,7-tetrahydro-5H-pyrazolo[4,3-c]pyridine-5-carboxamide (P-028)

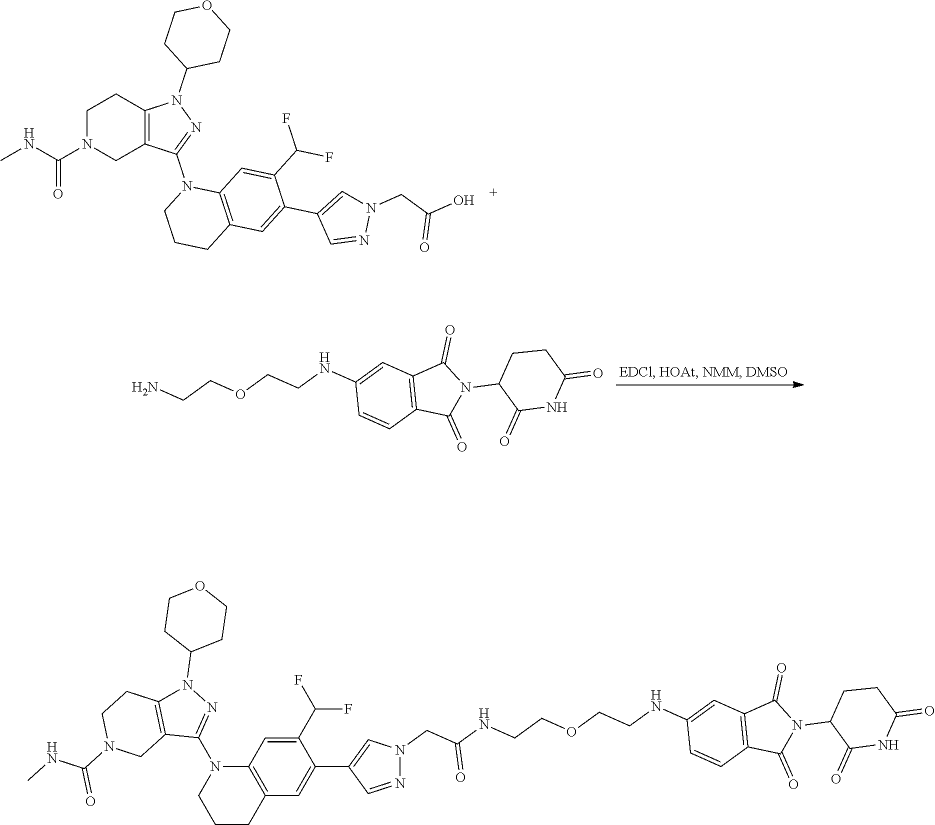 [SMILES: CNC(=O)N1CCc2c(c(N3CCCc4cc(-c5cnn(CC(=O)O)c5)c(C(F)F)cc43)nn2C2CCOCC2)C1 ; NCCOCCNc1ccc2c(c1)C(=O)N(C1CCC(=O)NC1=O)C2=O ; CNC(=O)N1CCc2c(c(N3CCCc4cc(-c5cnn(CC(=O)NCCOCCNc6ccc7c(c6)C(=O)N(C6CCC(=O)NC6=O)C7=O)c5)c(C(F)F)cc43)nn2C2CCOCC2)C1]

P-028 was synthesized following the standard procedure for preparing P-001 (4.3 mg, yield 31%). MS (ESI) m/z: 912.9 [M+H]⁺.

### Example 114. 3-(7-(Difluoromethyl)-6-(1-(24(2-(2-(2-((2-(2,6-dioxopiperidin-3-yl)-1,3-dioxoisoindolin-5-yl)amino)ethoxy)ethoxy)ethyl)amino)-2-oxoethyl)-1H-pyrazol-4-yl)-3,4-dihydroquinolin-1(2H)-yl)-N-methyl-1-(tetrahydro-2H-pyran-4-yl)-1,4,6,7-tetrahydro-5H-pyrazolo[4,3-c]pyridine-5-carboxamide (P-029)

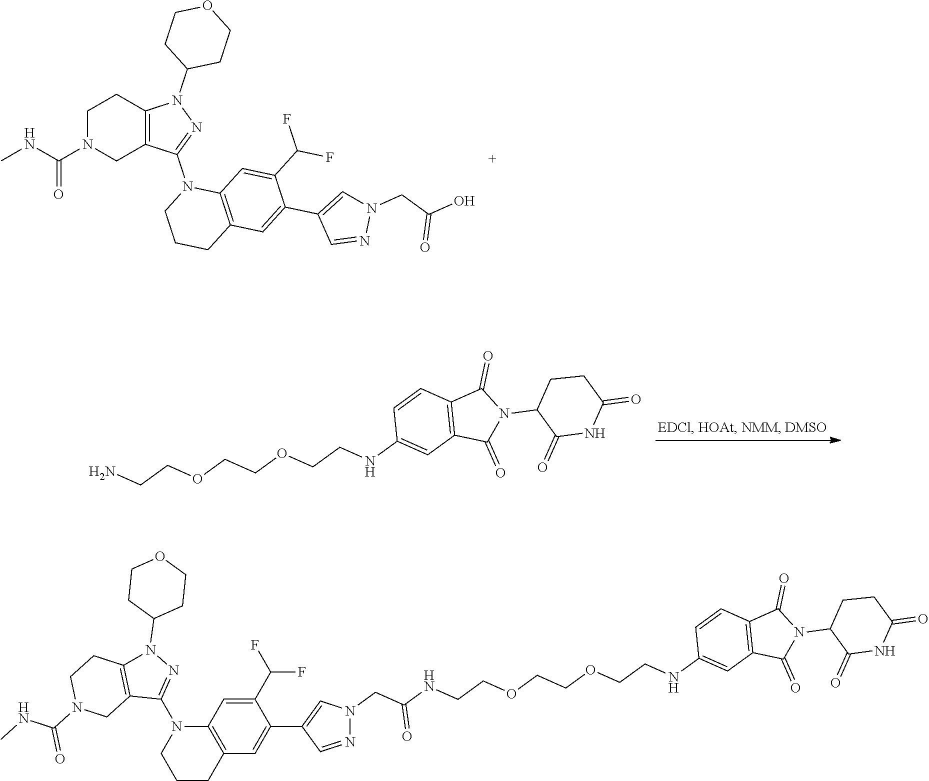 [SMILES: CNC(=O)N1CCc2c(c(N3CCCc4cc(-c5cnn(CC(=O)O)c5)c(C(F)F)cc43)nn2C2CCOCC2)C1 ; NCCOCCOCCNc1ccc2c(c1)C(=O)N(C1CCC(=O)NC1=O)C2=O ; CNC(=O)N1CCc2c(c(N3CCCc4cc(-c5cnn(CC(=O)NCCOCCOCCNc6ccc7c(c6)C(=O)N(C6CCC(=O)NC6=O)C7=O)c5)c(C(F)F)cc43)nn2C2CCOCC2)C1]

P-029 was synthesized following the standard procedure for preparing P-001 (4.6 mg, yield 32%). MS (ESI) m/z: 956.9 [M+H]⁺.

### Example 115. 3-(7-(Difluoromethyl)-6-(1-(14-((2-(2,6-dioxopiperidin-3-yl)-1,3-dioxoisoindolin-5-yl)amino)-2-oxo-6,9,12-trioxa-3-azatetradecyl)-1H-pyrazol-4-yl)-3,4-dihydroquinolin-1(2H)-yl)-N-methyl-1-(tetrahydro-2H-pyran-4-yl)-1,4,6,7-tetrahydro-5H-pyrazolo[4,3-c]pyridine-5-carboxamide (P-030)

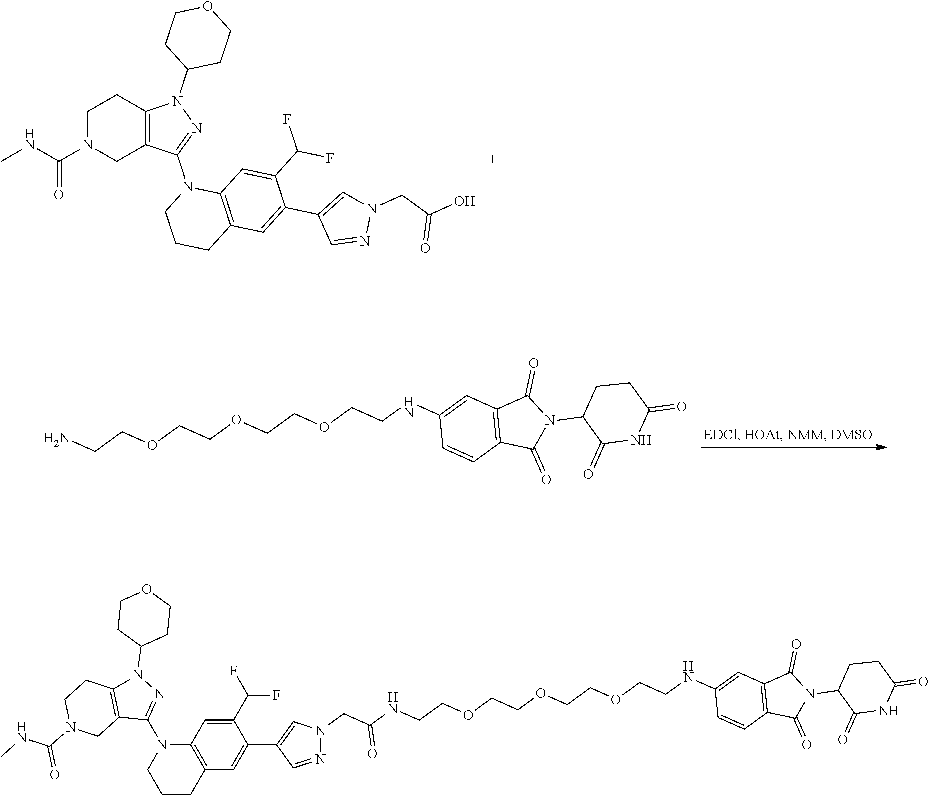 [SMILES: CNC(=O)N1CCc2c(c(N3CCCc4cc(-c5cnn(CC(=O)O)c5)c(C(F)F)cc43)nn2C2CCOCC2)C1 ; NCCOCCOCCOCCNc1ccc2c(c1)C(=O)N(C1CCC(=O)NC1=O)C2=O ; CNC(=O)N1CCc2c(c(N3CCCc4cc(-c5cnn(CC(=O)NCCOCCOCCOCCNc6ccc7c(c6)C(=O)N(C6CCC(=O)NC6=O)C7=O)c5)c(C(F)F)cc43)nn2C2CCOCC2)C1]

P-030 was synthesized following the standard procedure for preparing P-001 (6.4 mg, yield 43%). MS (ESI) m/z: 1001.1 [M+H]⁺.

### Example 116. 3-(7-(Difluoromethyl)-6-(1-(17-((2-(2,6-dioxopiperidin-3-yl)-1,3-dioxoisoindolin-5-yl)amino)-2-oxo-6,9,12,15-tetraoxa-3-azaheptadecyl)-1H-pyrazol-4-yl)-3,4-dihydroquinolin-1(2H)-yl)-N-methyl-1-(tetrahydro-2H-pyran-4-yl)-1,4,6,7-tetrahydro-5H-pyrazolo[4,3-c]pyridine-5-carboxamide (P-031)

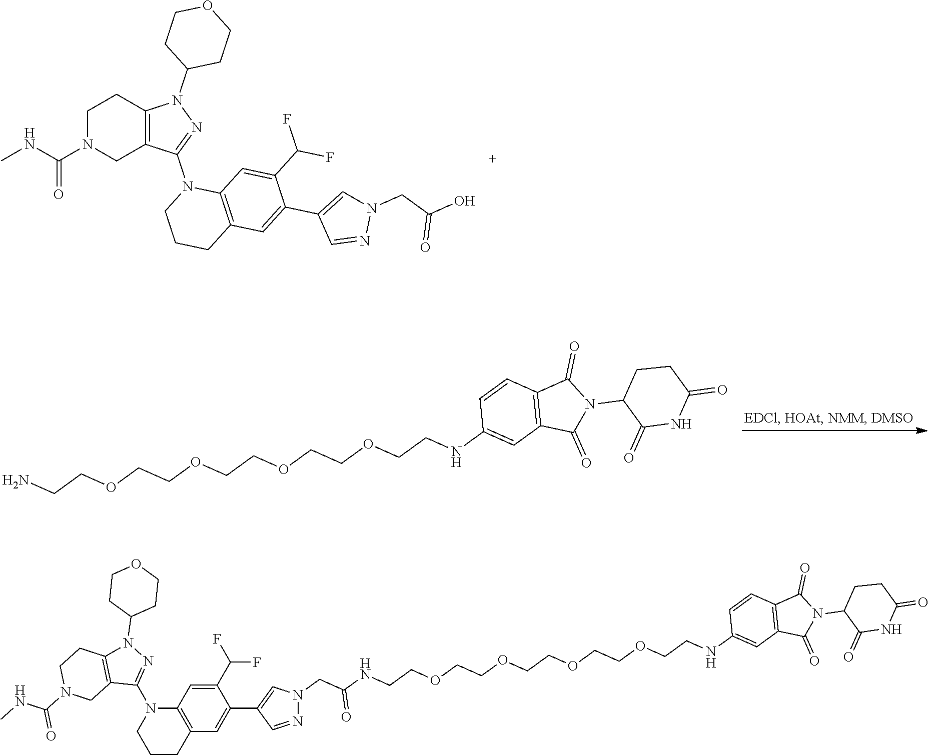 [SMILES: CNC(=O)N1CCc2c(c(N3CCCc4cc(-c5cnn(CC(=O)O)c5)c(C(F)F)cc43)nn2C2CCOCC2)C1 ; NCCOCCOCCOCCOCCNc1ccc2c(c1)C(=O)N(C1CCC(=O)NC1=O)C2=O ; CNC(=O)N1CCc2c(c(N3CCCc4cc(-c5cnn(CC(=O)NCCOCCOCCOCCOCCNc6ccc7c(c6)C(=O)N(C6CCC(=O)NC6=O)C7=O)c5)c(C(F)F)cc43)nn2C2CCOCC2)C1]

P-031 was synthesized following the standard procedure for preparing P-001 (5.3 mg, yield 34%). MS (ESI) m/z: 1045.1 [M+H]⁺.

### Example 117. 3-(7-(Difluoromethyl)-6-(1-(20-((2-(2,6-dioxopiperidin-3-yl)-1,3-dioxoisoindolin-5-yl)amino)-2-oxo-6,9,12,15,18-pentaoxa-3-azaicosyl)-1H-pyrazol-4-yl)-3,4-dihydroquinolin-1(2H)-yl)-N-methyl-1-(tetrahydro-2H-pyran-4-yl)-1,4,6,7-tetrahydro-5H-pyrazolo[4,3-c]pyridine-5-carboxamide (P-032)

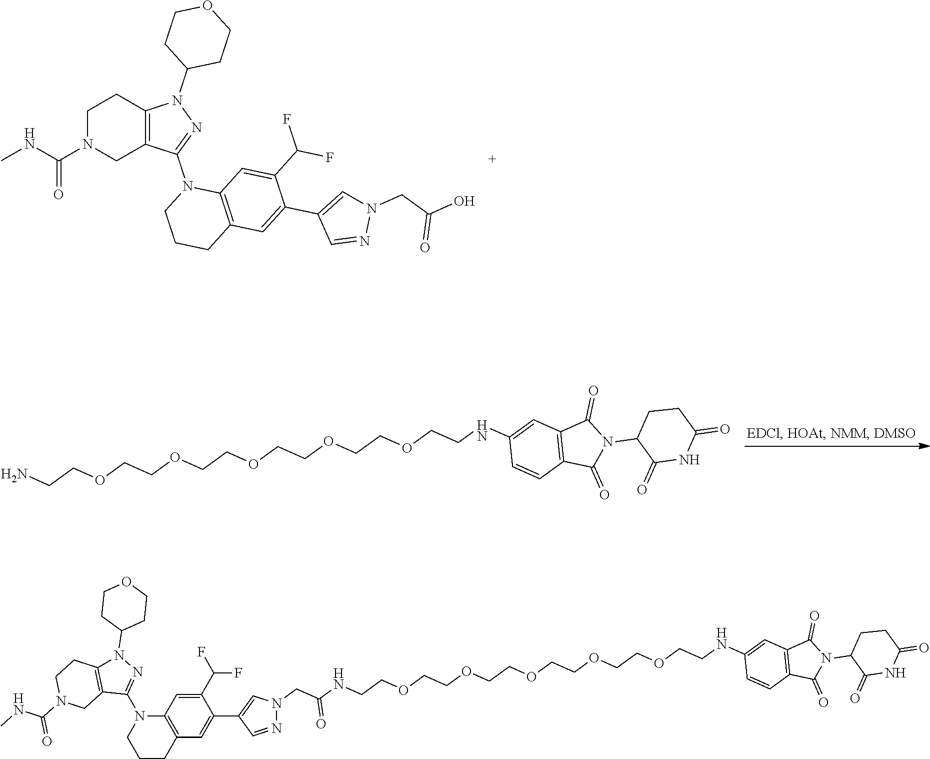 [SMILES: CNC(=O)N1CCc2c(c(N3CCCc4cc(-c5cnn(CC(=O)O)c5)c(C(F)F)cc43)nn2C2CCOCC2)C1 ; NCCOCCOCCOCCOCCOCCNc1ccc2c(c1)C(=O)N(C1CCC(=O)NC1=O)C2=O ; CNC(=O)N1CCc2c(c(N3CCCc4cc(-c5cnn(CC(=O)NCCOCCOCCOCCOCCOCCNc6ccc7c(c6)C(=O)N(C6CCC(=O)NC6=O)C7=O)c5)c(C(F)F)cc43)nn2C2CCOCC2)C1]

P-032 was synthesized following the standard procedure for preparing P-001 (6.7 mg, yield 41%). MS (ESI) m/z: 1089.2 [M+H]⁺.

### Example 118. 3-(7-(Difluoromethyl)-6-(1-methyl-1H-pyrazol-4-yl)-3,4-dihydroquinolin-1(2H)-yl)-1-(1-(5-((2-(2,6-dioxopiperidin-3-yl)-1,3-dioxoisoindolin-4-yl)amino)pentanoyl)piperidin-4-yl)-N-methyl-1,4,6,7-tetrahydro-5H-pyrazolo[4,3-c]pyridine-5-carboxamide (P-033)

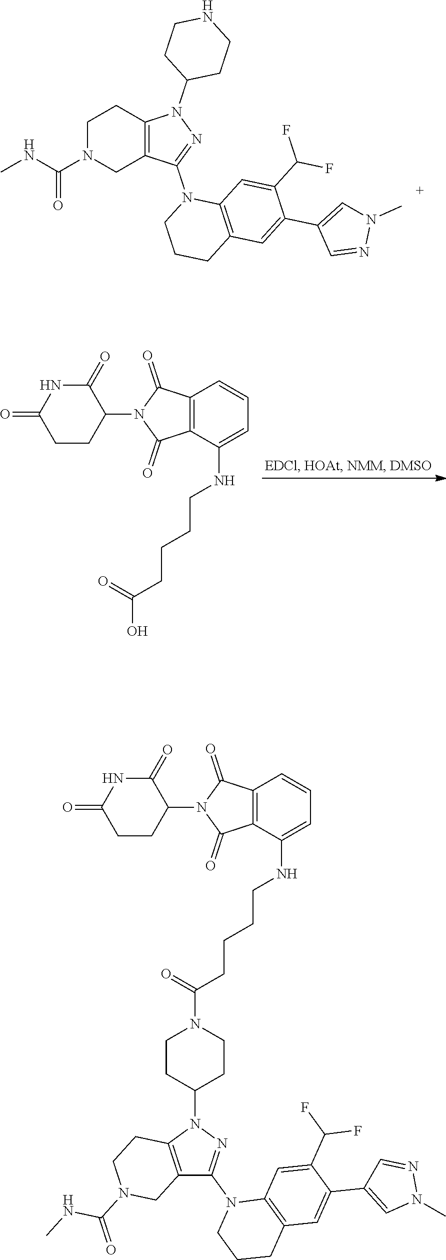

P-033 was synthesized following the standard procedure for preparing P-001 (1.4 mg, yield 11%). MS (ESI) m/z: 881.0 [M+H]⁺.

### Example 119. 3-(7-(Difluoromethyl)-6-(1-methyl-1H-pyrazol-4-yl)-3,4-dihydroquinolin-1(2H)-yl)-1-(1-(6-((2-(2,6-dioxopiperidin-3-yl)-1,3-dioxoisoindolin-4-yl)amino)hexanoyl)piperidin-4-yl)-N-methyl-1,4,6,7-tetrahydro-5H-pyrazolo[4,3-c]pyridine-5-carboxamide (P-034)

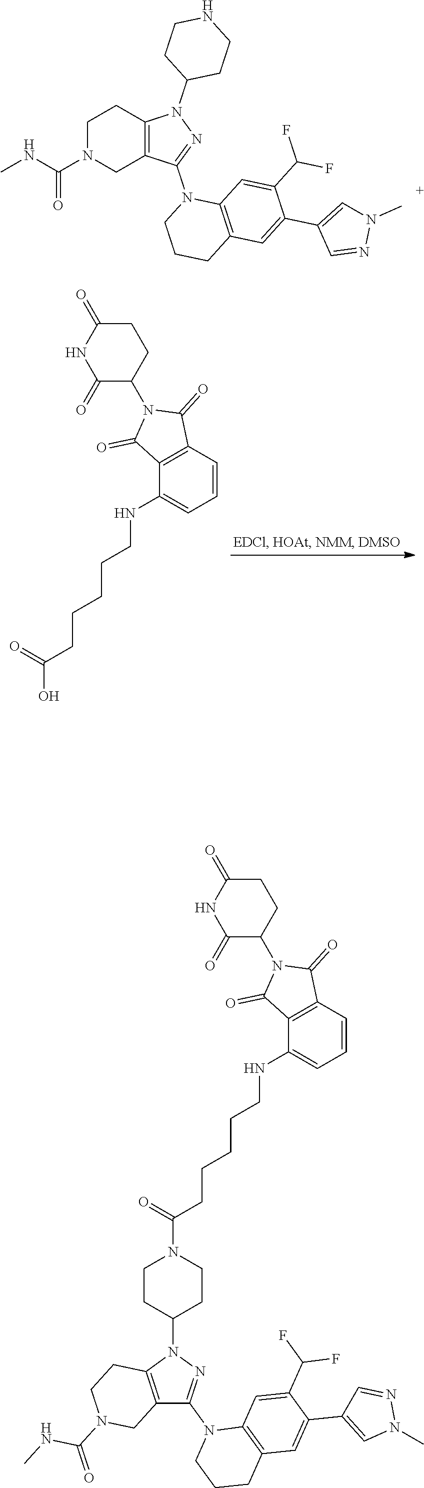

P-034 was synthesized following the standard procedure for preparing P-001 (1.3 mg, yield 10%). MS (ESI) m/z: 894.9 [M+H]⁺.

### Example 120. 3-(7-(Difluoromethyl)-6-(1-methyl-1H-pyrazol-4-yl)-3,4-dihydroquinolin-1(2H)-yl)-1-(1-(7-((2-(2,6-dioxopiperidin-3-yl)-1,3-dioxoisoindolin-4-yl)amino)heptanoyl)piperidin-4-yl)-N-methyl-1,4,6,7-tetrahydro-5H-pyrazolo[4,3-c]pyridine-5-carboxamide (P-035)

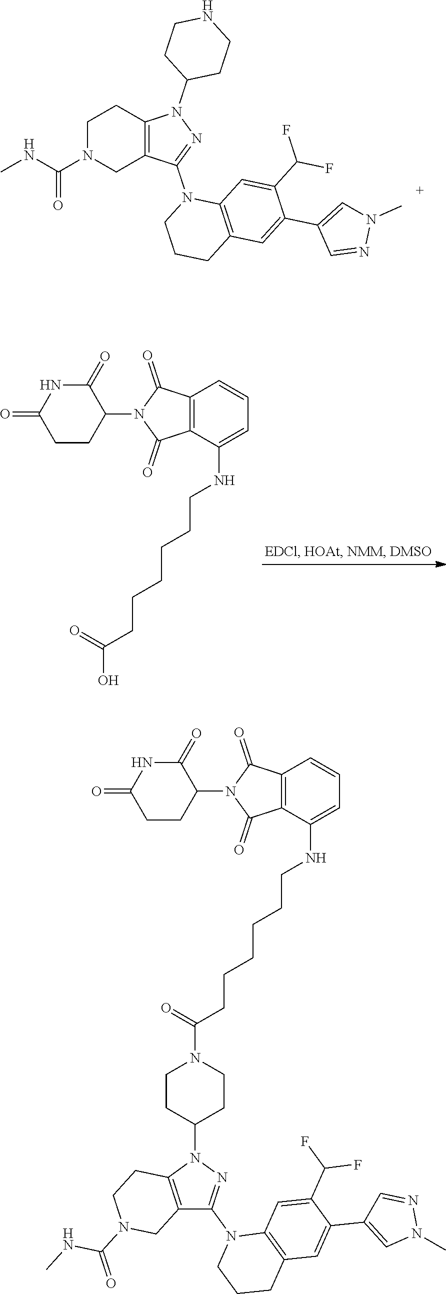

P-035 was synthesized following the standard procedure for preparing P-001 (1.55 mg, yield 11%). MS (ESI) m/z: 908.8 [M+H]⁺.

### Example 121. 3-(7-(Difluoromethyl)-6-(1-methyl-1H-pyrazol-4-yl)-3,4-dihydroquinolin-1(2H)-yl)-1-(1-(8-((2-(2,6-dioxopiperidin-3-yl)-1,3-dioxoisoindolin-4-yl)amino)octanoyl)piperidin-4-yl)-N-methyl-1,4,6,7-tetrahydro-5H-pyrazolo[4,3-c]pyridine-5-carboxamide (P-036)

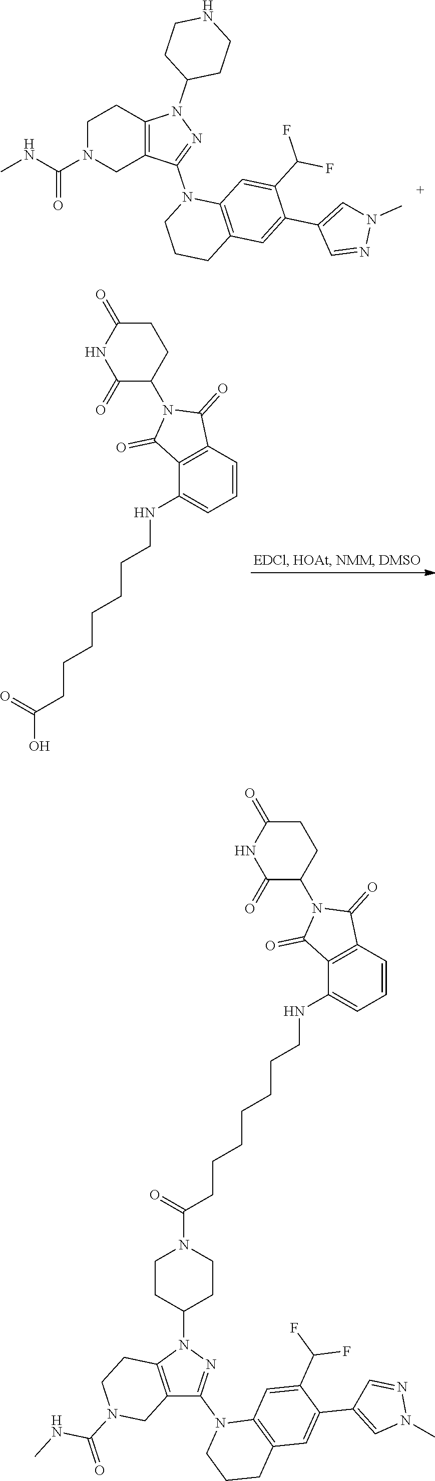

P-036 was synthesized following the standard procedure for preparing P-001 (2.31 mg, yield 17%). MS (ESI) m/z: 922.8 [M+H]⁺.

### Example 122. 3-(7-(Difluoromethyl)-6-(1-(2-((2-((2-(2,6-dioxopiperidin-3-yl)-1,3-dioxoisoindolin-4-yl)amino)ethyl)amino)-2-oxoethyl)-1H-pyrazol-4-yl)-3,4-dihydroquinolin-1(2H)-yl)-N-methyl-1-(tetrahydro-2H-pyran-4-yl)-1,4,6,7-tetrahydro-5H-pyrazolo[4,3-c]pyridine-5-carboxamide (P-037)

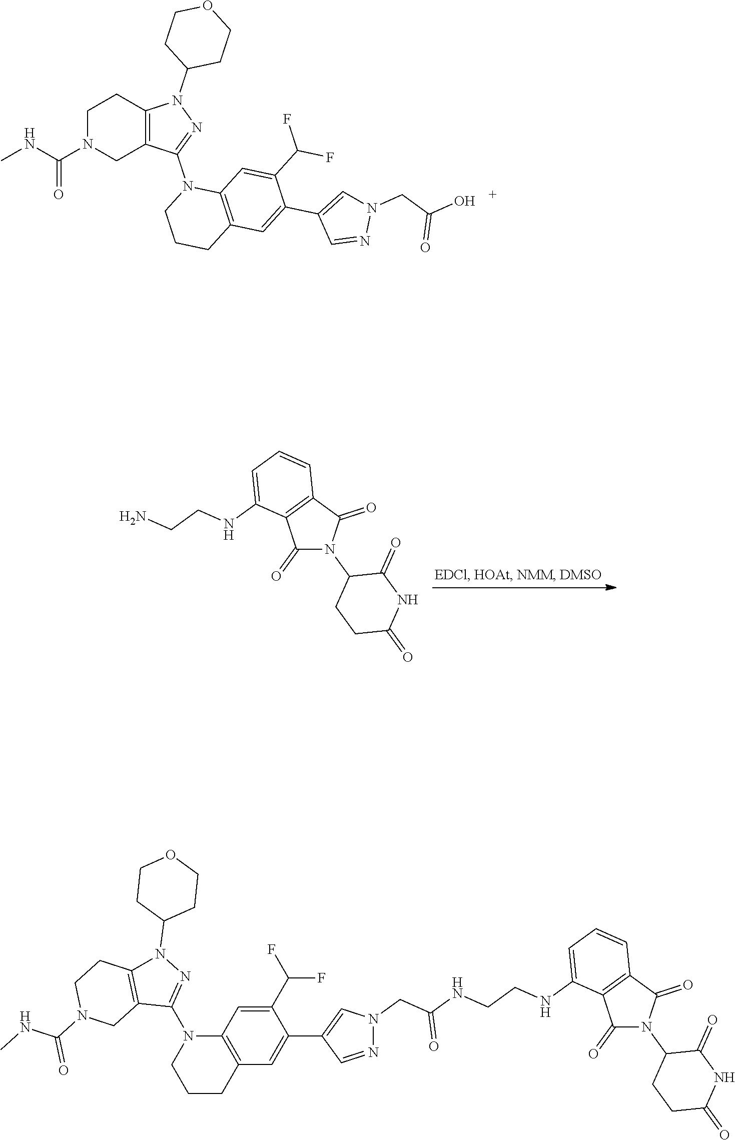 [SMILES: CNC(=O)N1CCc2c(c(N3CCCc4cc(-c5cnn(CC(=O)O)c5)c(C(F)F)cc43)nn2C2CCOCC2)C1 ; NCCNc1cccc2c1C(=O)N(C1CCC(=O)NC1=O)C2=O ; CNC(=O)N1CCc2c(c(N3CCCc4cc(-c5cnn(CC(=O)NCCNc6cccc7c6C(=O)N(C6CCC(=O)NC6=O)C7=O)c5)c(C(F)F)cc43)nn2C2CCOCC2)C1]

P-037 was synthesized following the standard procedure for preparing P-001 (3 mg, yield 23%). MS (ESI) m/z: 868.8 [M+H]⁺.

### Example 123. 3-(7-(Difluoromethyl)-6-(1-(2-((3-((2-(2,6-dioxopiperidin-3-yl)-1,3-dioxoisoindolin-4-yl)amino)propyl)amino)-2-oxoethyl)-1H-pyrazol-4-yl)-3,4-dihydroquinolin-1(2H)-yl)-N-methyl-1-(tetrahydro-2H-pyran-4-yl)-1,4,6,7-tetrahydro-5H-pyrazolo[4,3-c]pyridine-5-carboxamide (P-038)

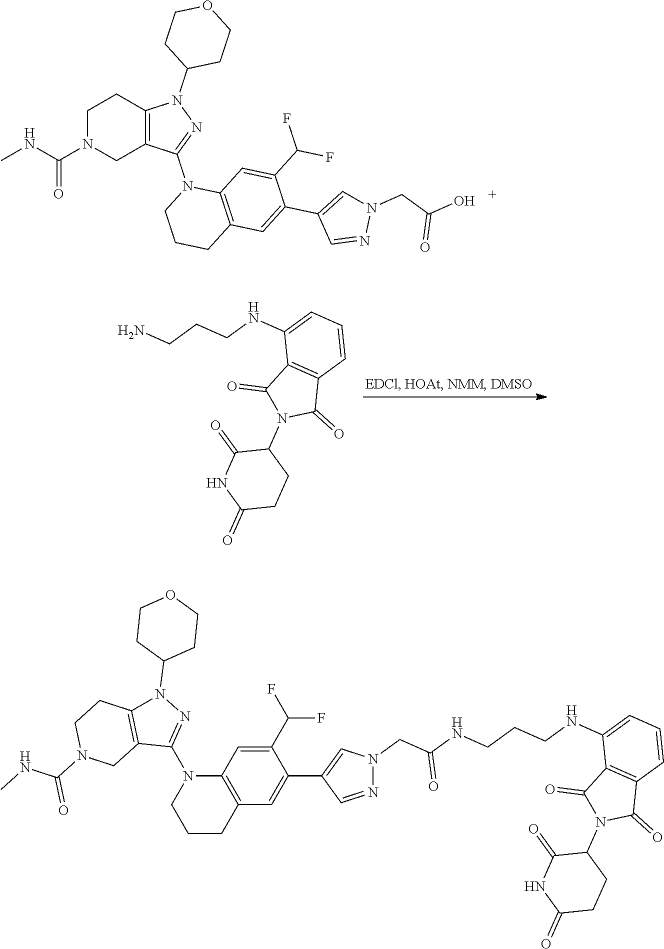 [SMILES: CNC(=O)N1CCc2c(c(N3CCCc4cc(-c5cnn(CC(=O)O)c5)c(C(F)F)cc43)nn2C2CCOCC2)C1 ; NCCCNc1cccc2c1C(=O)N(C1CCC(=O)NC1=O)C2=O ; CNC(=O)N1CCc2c(c(N3CCCc4cc(-c5cnn(CC(=O)NCCCNc6cccc7c6C(=O)N(C6CCC(=O)NC6=O)C7=O)c5)c(C(F)F)cc43)nn2C2CCOCC2)C1]

P-038 was synthesized following the standard procedure for preparing P-001 (4.5 mg, yield 34%). MS (ESI) m/z: 882.8 [M+H]⁺.

### Example 124. 3-(7-(Difluoromethyl)-6-(1-(2-((4-((2-(2,6-dioxopiperidin-3-yl)-1,3-dioxoisoindolin-4-yl)amino)butyl)amino)-2-oxoethyl)-1H-pyrazol-4-yl)-3,4-dihydroquinolin-1(2H)-yl)-N-methyl-1-(tetrahydro-2H-pyran-4-yl)-1,4,6,7-tetrahydro-5H-pyrazolo[4,3-c]pyridine-5-carboxamide (P-039)

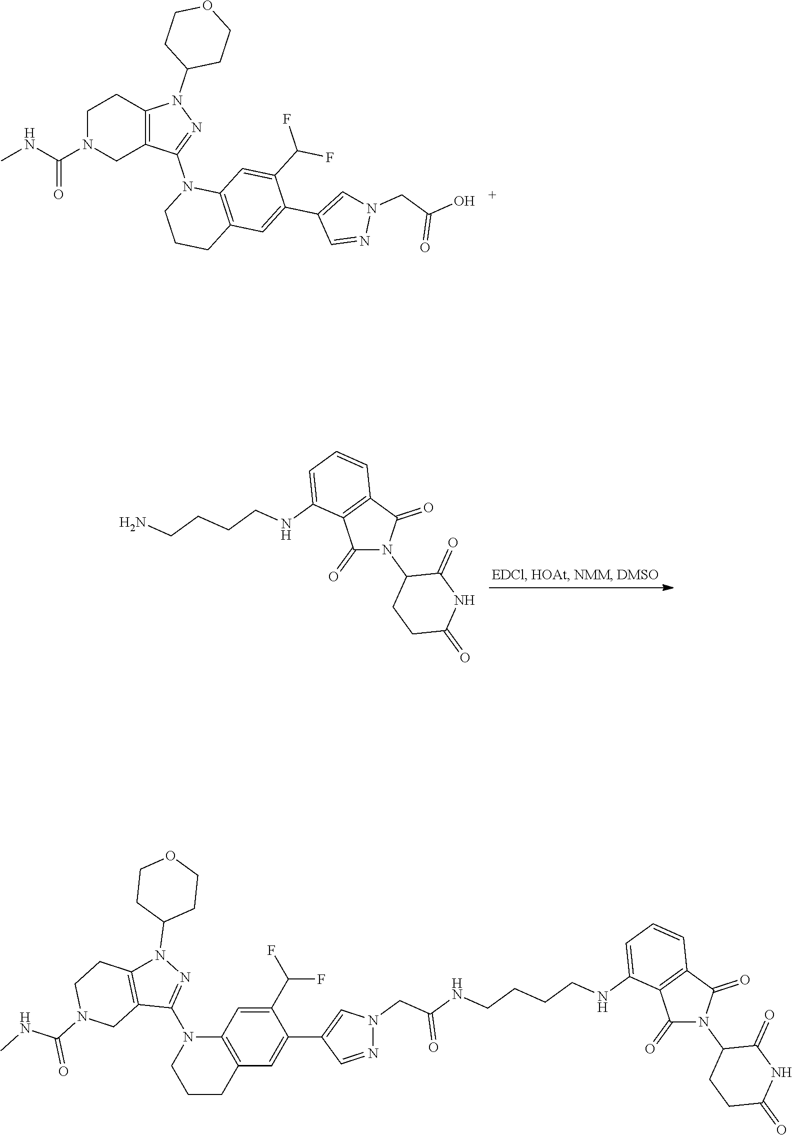 [SMILES: CNC(=O)N1CCc2c(c(N3CCCc4cc(-c5cnn(CC(=O)O)c5)c(C(F)F)cc43)nn2C2CCOCC2)C1 ; NCCCCNc1cccc2c1C(=O)N(C1CCC(=O)NC1=O)C2=O ; CNC(=O)N1CCc2c(c(N3CCCc4cc(-c5cnn(CC(=O)NCCCCNc6cccc7c6C(=O)N(C6CCC(=O)NC6=O)C7=O)c5)c(C(F)F)cc43)nn2C2CCOCC2)C1]

P-039 was synthesized following the standard procedure for preparing P-001 (5.6 mg, yield 42%). MS (ESI) m/z: 896.9 [M+H]⁺.

### Example 125. 3-(7-(Difluoromethyl)-6-(1-(2-((5-((2-(2,6-dioxopiperidin-3-yl)-1,3-dioxoisoindolin-4-yl)amino)pentyl)amino)-2-oxoethyl)-1H-pyrazol-4-yl)-3,4-dihydroquinolin-1(2H)-yl)-N-methyl-1-(tetrahydro-2H-pyran-4-yl)-1,4,6,7-tetrahydro-5H-pyrazolo[4,3-c]pyridine-5-carboxamide (P-040)

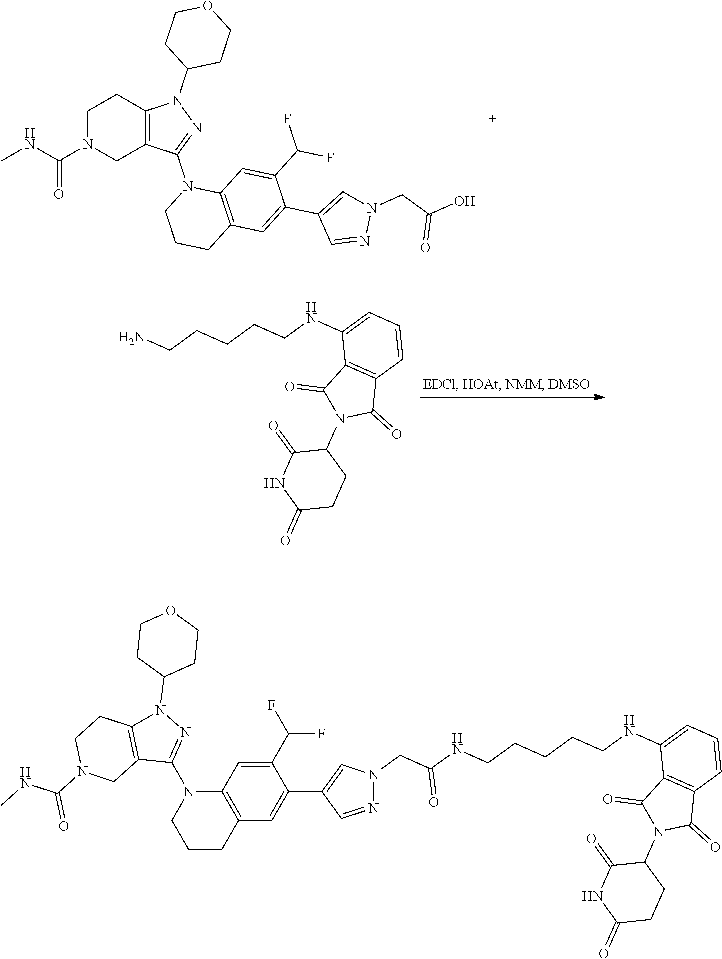 [SMILES: CNC(=O)N1CCc2c(c(N3CCCc4cc(-c5cnn(CC(=O)O)c5)c(C(F)F)cc43)nn2C2CCOCC2)C1 ; NCCCCCNc1cccc2c1C(=O)N(C1CCC(=O)NC1=O)C2=O ; CNC(=O)N1CCc2c(c(N3CCCc4cc(-c5cnn(CC(=O)NCCCCCNc6cccc7c6C(=O)N(C6CCC(=O)NC6=O)C7=O)c5)c(C(F)F)cc43)nn2C2CCOCC2)C1]

P-040 was synthesized following the standard procedure for preparing P-001 (5.0 mg, yield 37%). MS (ESI) m/z: 910.8 [M+H]⁺.

### Example 126. 3-(7-(Difluoromethyl)-6-(1-(2-((6-((2-(2,6-dioxopiperidin-3-yl)-1,3-dioxoisoindolin-4-yl)amino)hexyl)amino)-2-oxoethyl)-1H-pyrazol-4-yl)-3,4-dihydroquinolin-1(2H)-yl)-N-methyl-1-(tetrahydro-2H-pyran-4-yl)-1,4,6,7-tetrahydro-5H-pyrazolo[4,3-c]pyridine-5-carboxamide (P-041)

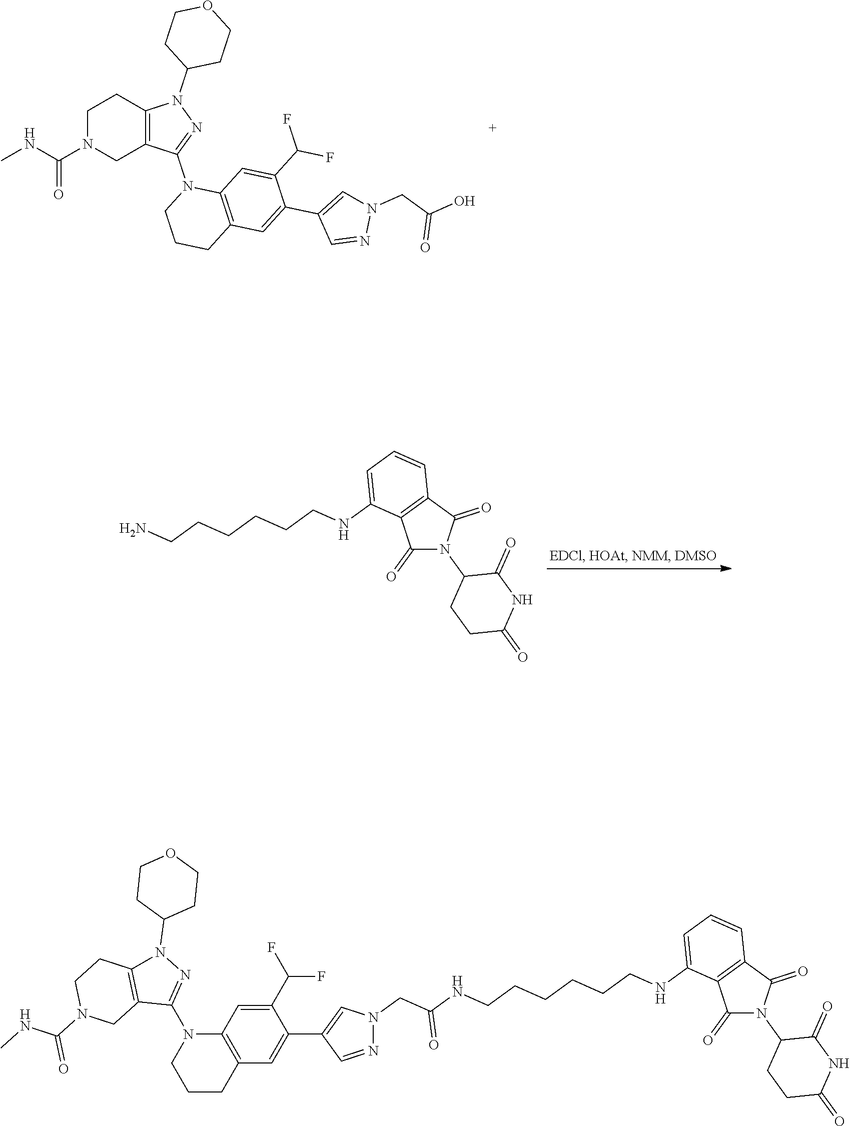 [SMILES: CNC(=O)N1CCc2c(c(N3CCCc4cc(-c5cnn(CC(=O)O)c5)c(C(F)F)cc43)nn2C2CCOCC2)C1 ; NCCCCCCNc1cccc2c1C(=O)N(C1CCC(=O)NC1=O)C2=O ; CNC(=O)N1CCc2c(c(N3CCCc4cc(-c5cnn(CC(=O)NCCCCCCNc6cccc7c6C(=O)N(C6CCC(=O)NC6=O)C7=O)c5)c(C(F)F)cc43)nn2C2CCOCC2)C1]

P-041 was synthesized following the standard procedure for preparing P-026 (1.8 mg, yield 13%). MS (ESI) m/z: 924.7 [M+H]⁺.

### Example 127. 3-(7-(Difluoromethyl)-6-(1-(24(7-((2-(2,6-dioxopiperidin-3-yl)-1,3-dioxoisoindolin-4-yl)amino)heptyl)amino)-2-oxoethyl)-1H-pyrazol-4-yl)-3,4-dihydroquinolin-1(2H)-yl)-N-methyl-1-(tetrahydro-2H-pyran-4-yl)-1,4,6,7-tetrahydro-5H-pyrazolo[4,3-c]pyridine-5-carboxamide (P-042)

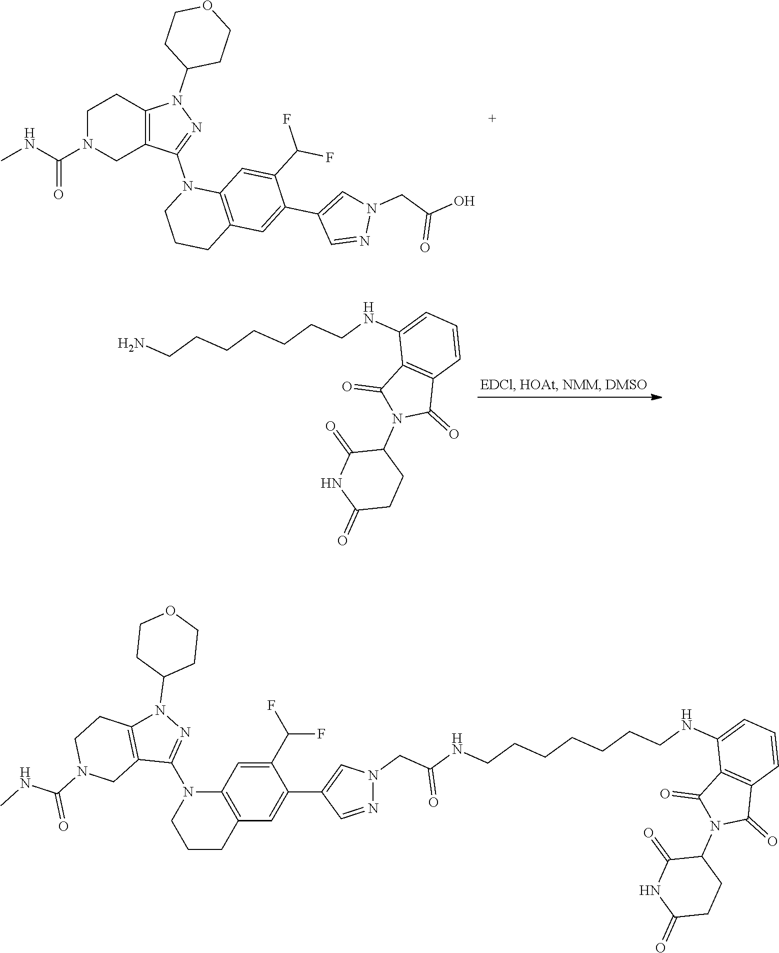 [SMILES: CNC(=O)N1CCc2c(c(N3CCCc4cc(-c5cnn(CC(=O)O)c5)c(C(F)F)cc43)nn2C2CCOCC2)C1 ; NCCCCCCCNc1cccc2c1C(=O)N(C1CCC(=O)NC1=O)C2=O ; CNC(=O)N1CCc2c(c(N3CCCc4cc(-c5cnn(CC(=O)NCCCCCCCNc6cccc7c6C(=O)N(C6CCC(=O)NC6=O)C7=O)c5)c(C(F)F)cc43)nn2C2CCOCC2)C1]

P-042 was synthesized following the standard procedure for preparing P-001 (2.8 mg, yield 20%). MS (ESI) m/z: 938.9 [M+H]⁺.

### Example 128. 3-(7-(Difluoromethyl)-6-(1-(2-((8-((2-(2,6-dioxopiperidin-3-yl)-1,3-dioxoisoindolin-4-yl)amino)octyl)amino)-2-oxoethyl)-1H-pyrazol-4-yl)-3,4-dihydroquinolin-1(2H)-yl)-N-methyl-1-(tetrahydro-2H-pyran-4-yl)-1,4,6,7-tetrahydro-5H-pyrazolo[4,3-c]pyridine-5-carboxamide (P-043)

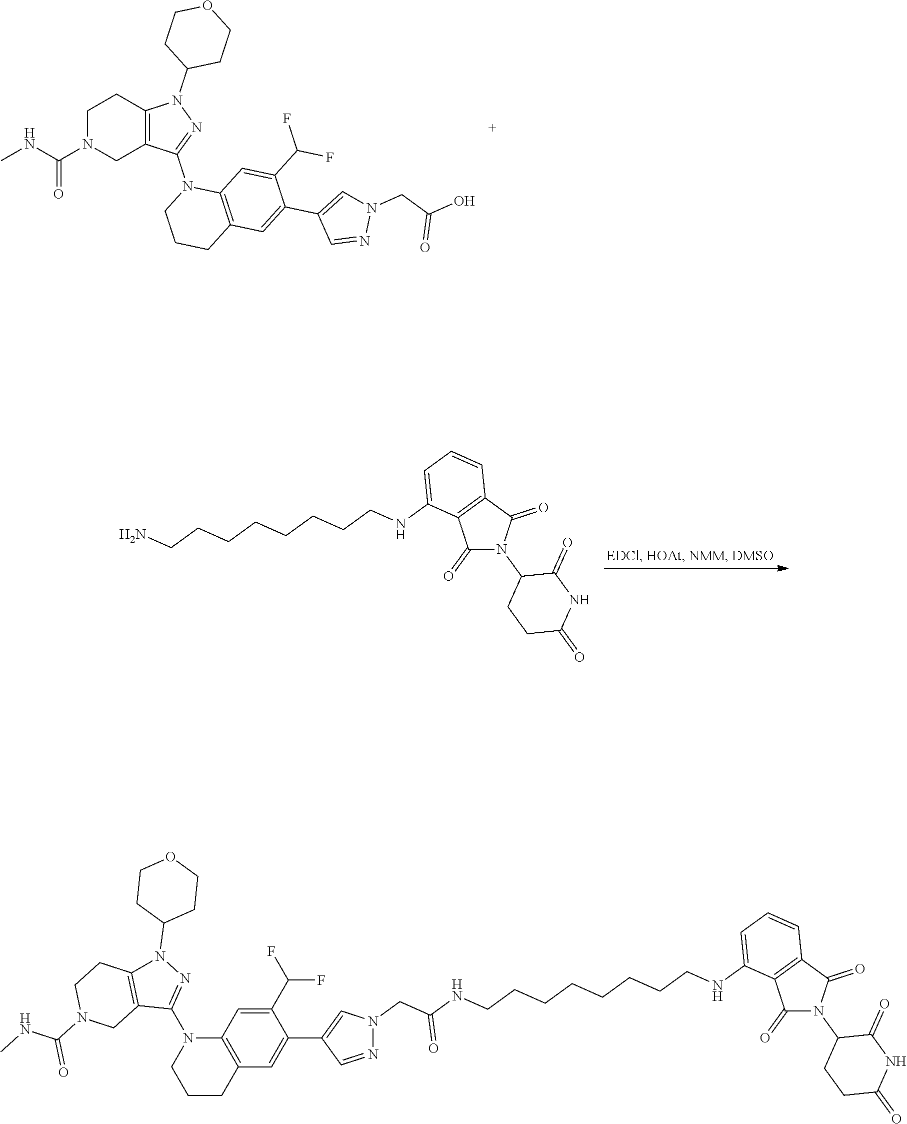 [SMILES: CNC(=O)N1CCc2c(c(N3CCCc4cc(-c5cnn(CC(=O)O)c5)c(C(F)F)cc43)nn2C2CCOCC2)C1 ; NCCCCCCCCNc1cccc2c1C(=O)N(C1CCC(=O)NC1=O)C2=O ; CNC(=O)N1CCc2c(c(N3CCCc4cc(-c5cnn(CC(=O)NCCCCCCCCNc6cccc7c6C(=O)N(C6CCC(=O)NC6=O)C7=O)c5)c(C(F)F)cc43)nn2C2CCOCC2)C1]

P-043 was synthesized following the standard procedure for preparing P-001 (3.7 mg, yield 26%). MS (ESI) m/z: 953.0 [M+H]⁺.

### Example 129. 3-(7-(Difluoromethyl)-6-(1-(24(2-(((S)-1-((2S,4R)-4-hydroxy-2-((4-(4-methylthiazol-5-yl)benzyl)carbamoyl)pyrrolidin-1-yl)-3,3-dimethyl-1-oxobutan-2-yl)amino)-2-oxoethyl)amino)-2-oxoethyl)-1H-pyrazol-4-yl)-3,4-dihydroquinolin-1(2H)-yl)-N-methyl-1-(tetrahydro-2H-pyran-4-yl)-1,4,6,7-tetrahydro-5H-pyrazolo[4,3-c]pyridine-5-carboxamide (P-044)

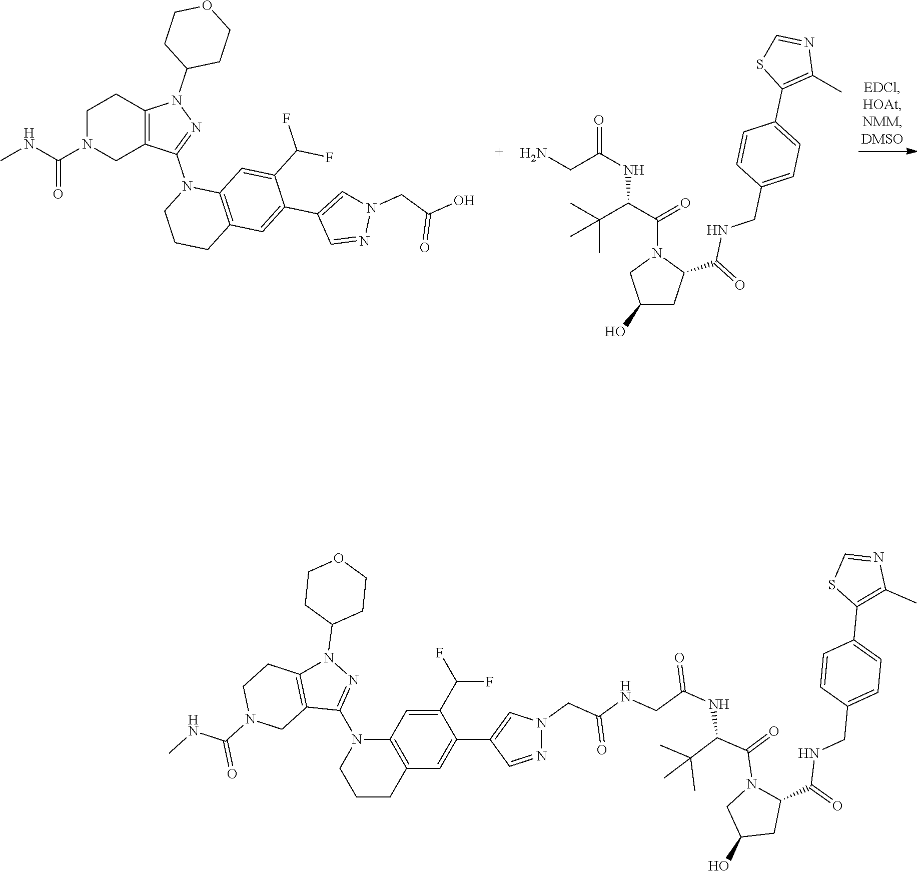

P-044 was synthesized following the standard procedure for preparing P-001 (4.4 mg, yield 28%). MS (ESI) m/z: 1040.0 [M+H]⁺.

### Example 130. 3-(7-(Difluoromethyl)-6-(1-(2-((3-(((S)-1-((2S,4R)-4-hydroxy-2-((4-(4-methylthiazol-5-yl)benzyl)carbamoyl)pyrrolidin-1-yl)-3,3-dimethyl-1-oxobutan-2-yl)amino)-3-oxopropyl)amino)-2-oxoethyl)-1H-pyrazol-4-yl)-3,4-dihydroquinolin-1(2H)-yl)-N-methyl-1-(tetrahydro-2H-pyran-4-yl)-1,4,6,7-tetrahydro-5H-pyrazolo[4,3-c]pyridine-5-carboxamide (P-045)

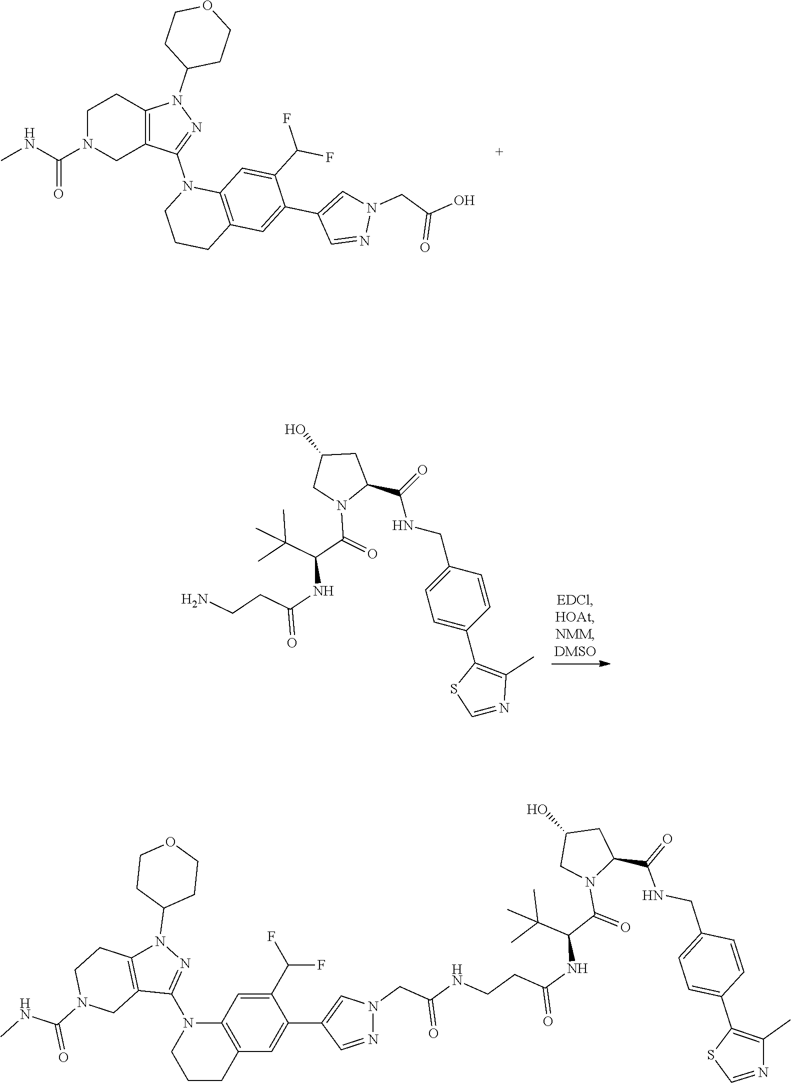

P-045 was synthesized following the standard procedure for preparing P-001 (3.7 mg, yield 23%). MS (ESI) m/z: 1054.1 [M+H]⁺.

### Example 131. 3-(7-(Difluoromethyl)-6-(1-(2-((5-(((S)-1-((2S,4R)-4-hydroxy-2-((4-(4-methylthiazol-5-yl)benzyl)carbamoyl)pyrrolidin-1-yl)-3,3-dimethyl-1-oxobutan-2-yl)amino)-5-oxopentyl)amino)-2-oxoethyl)-1H-pyrazol-4-yl)-3,4-dihydroquinolin-1(2H)-yl)-N-methyl-1-(tetrahydro-2H-pyran-4-yl)-1,4,6,7-tetrahydro-5H-pyrazolo[4,3-c]pyridine-5-carboxamide (P-046)

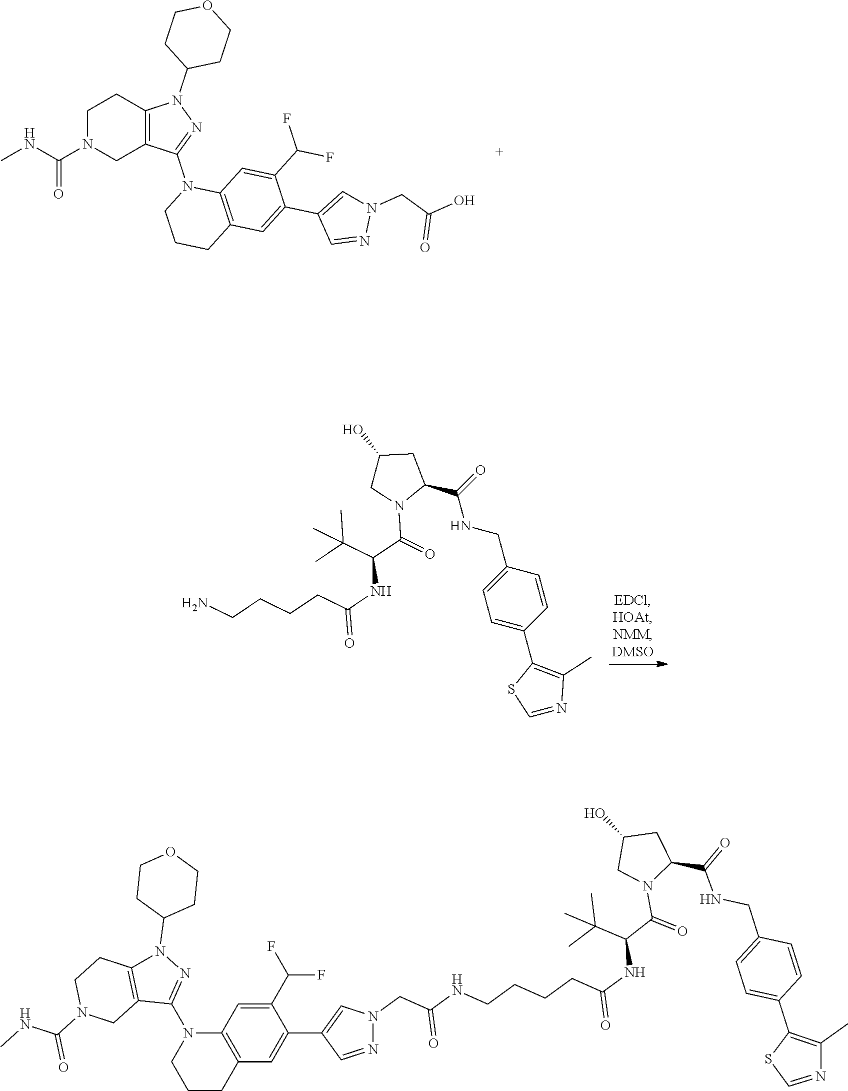

P-046 was synthesized following the standard procedure for preparing P-001 (2.2 mg, yield 14%). MS (ESI) m/z: 1082.1 [M+H]⁺.

### Example 132. 3-(7-(Difluoromethyl)-6-(1-(2-((6-(((S)-1-((2S,4R)-4-hydroxy-2-((4-(4-methylthiazol-5-yl)benzyl)carbamoyl)pyrrolidin-1-yl)-3,3-dimethyl-1-oxobutan-2-yl)amino)-6-oxohexyl)amino)-2-oxoethyl)-1H-pyrazol-4-yl)-3,4-dihydroquinolin-1(2H)-yl)-N-methyl-1-(tetrahydro-2H-pyran-4-yl)-1,4,6,7-tetrahydro-5H-pyrazolo[4,3-c]pyridine-5-carboxamide (P-047)

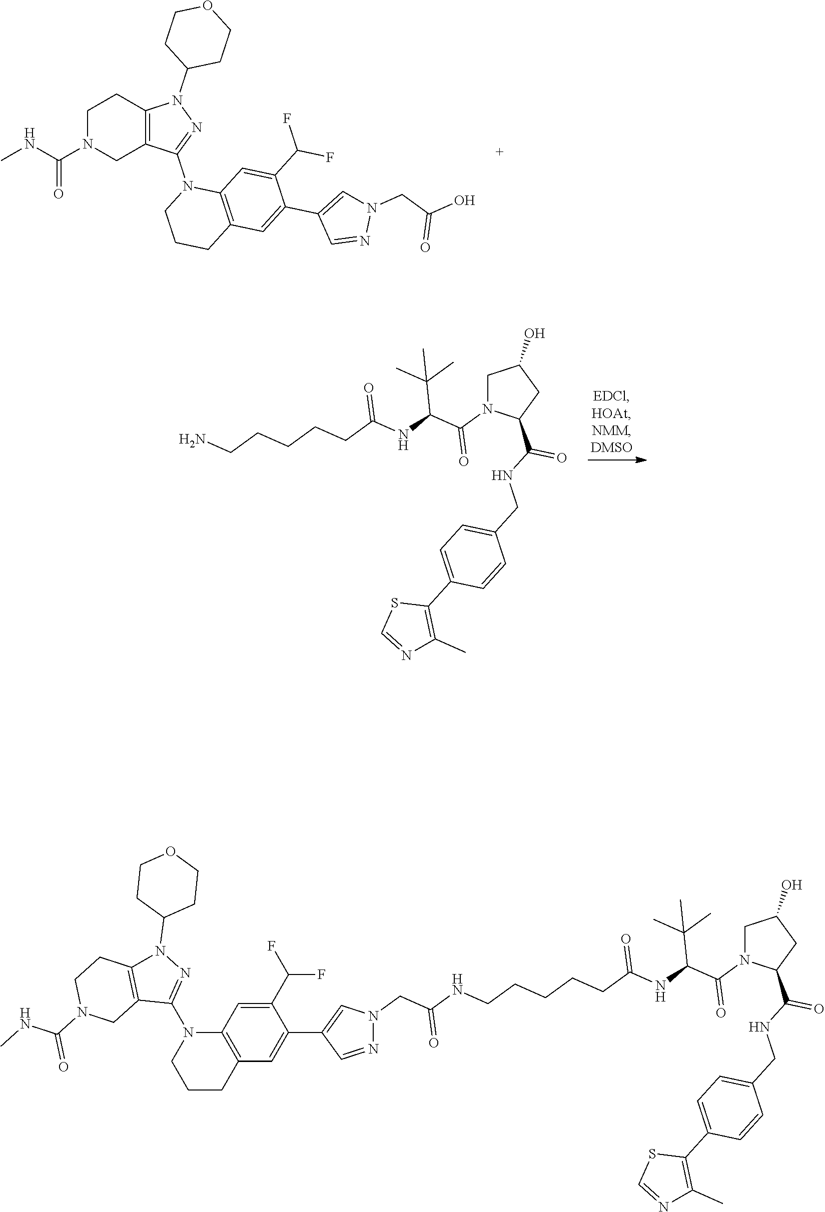 [SMILES: CNC(=O)N1CCc2c(c(N3CCCc4cc(-c5cnn(CC(=O)O)c5)c(C(F)F)cc43)nn2C2CCOCC2)C1 ; Cc1ncsc1-c1ccc(CNC(=O)[C@@H]2C[C@@H](O)CN2C(=O)[C@@H](NC(=O)CCCCCN)C(C)(C)C)cc1 ; CNC(=O)N1CCc2c(c(N3CCCc4cc(-c5cnn(CC(=O)NCCCCCC(=O)N[C@H](C(=O)N6C[C@H](O)C[C@H]6C(=O)NCc6ccc(-c7scnc7C)cc6)C(C)(C)C)c5)c(C(F)F)cc43)nn2C2CCOCC2)C1]

P-047 was synthesized following the standard procedure for preparing P-001 (2.0 mg, yield 12%). MS (ESI) m/z: 1096.1 [M+H]⁺.

### Example 133. 3-(7-(Difluoromethyl)-6-(1-(2-((8-(((S)-1-((2S,4R)-4-hydroxy-2-((4-(4-methylthiazol-5-yl)benzyl)carbamoyl)pyrrolidin-1-yl)-3,3-dimethyl-1-oxobutan-2-yl)amino)-8-oxooctyl)amino)-2-oxoethyl)-1H-pyrazol-4-yl)-3,4-dihydroquinolin-1(2H)-yl)-N-methyl-1-(tetrahydro-2H-pyran-4-yl)-1,4,6,7-tetrahydro-5H-pyrazolo[4,3-c]pyridine-5-carboxamide (P-048)

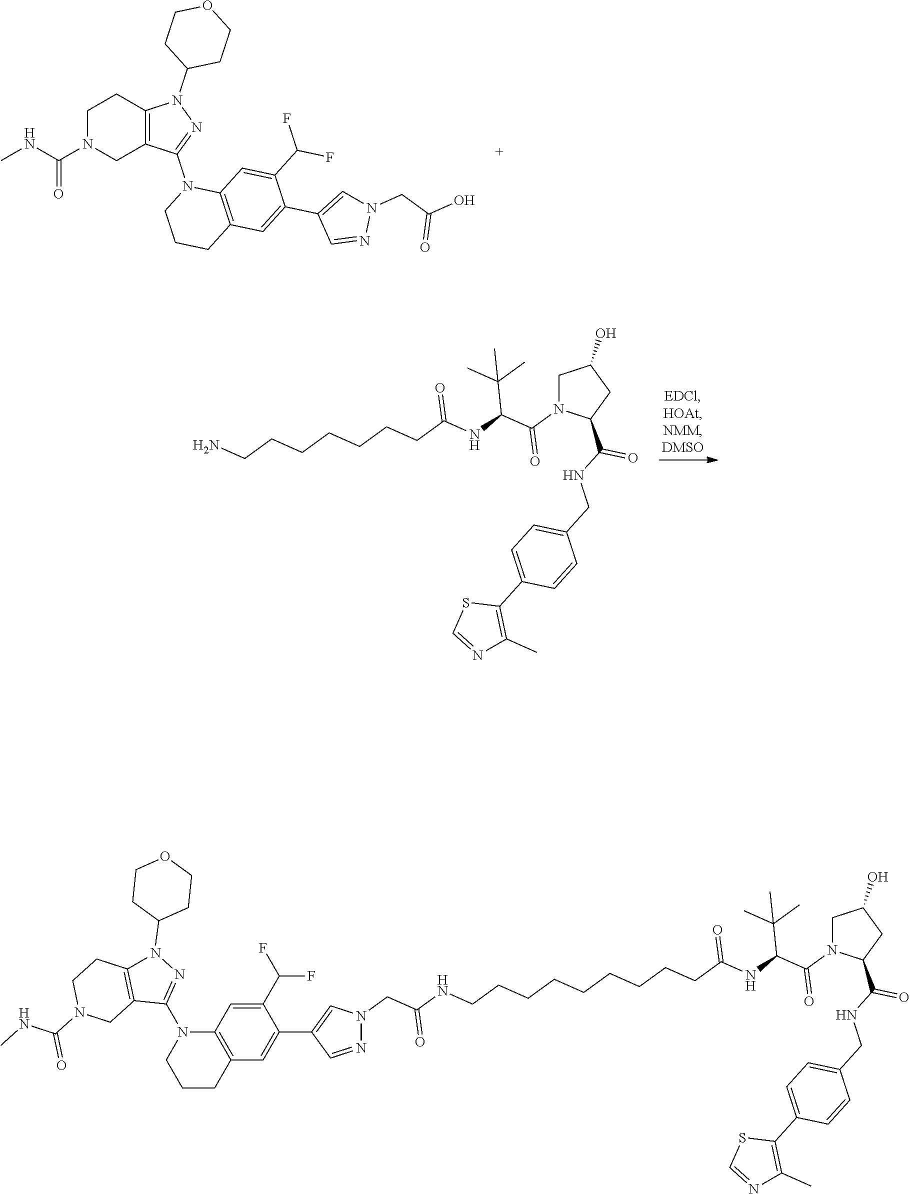 [SMILES: CNC(=O)N1CCc2c(c(N3CCCc4cc(-c5cnn(CC(=O)O)c5)c(C(F)F)cc43)nn2C2CCOCC2)C1 ; Cc1ncsc1-c1ccc(CNC(=O)[C@@H]2C[C@@H](O)CN2C(=O)[C@@H](NC(=O)CCCCCCCN)C(C)(C)C)cc1 ; CNC(=O)N1CCc2c(c(N3CCCc4cc(-c5cnn(CC(=O)NCCCCCCCCCC(=O)N[C@H](C(=O)N6C[C@H](O)C[C@H]6C(=O)NCc6ccc(-c7scnc7C)cc6)C(C)(C)C)c5)c(C(F)F)cc43)nn2C2CCOCC2)C1]

P-048 was synthesized following the standard procedure for preparing P-001 (1.05 mg, yield 6%). MS (ESI) m/z: 1124.1 [M+H]⁺.

### Example 134. 3-(7-(Difluoromethyl)-6-(1-(2-((10-(((S)-1-((2S,4R)-4-hydroxy-2-((4-(4-methylthiazol-5-yl)benzyl)carbamoyl)pyrrolidin-1-yl)-3,3-dimethyl-1-oxobutan-2-yl)amino)-10-oxodecyl)amino)-2-oxoethyl)-1H-pyrazol-4-yl)-3,4-dihydroquinolin-1(2H)-yl)-N-methyl-1-(tetrahydro-2H-pyran-4-yl)-1,4,6,7-tetrahydro-5H-pyrazolo[4,3-c]pyridine-5-carboxamide (P-049)

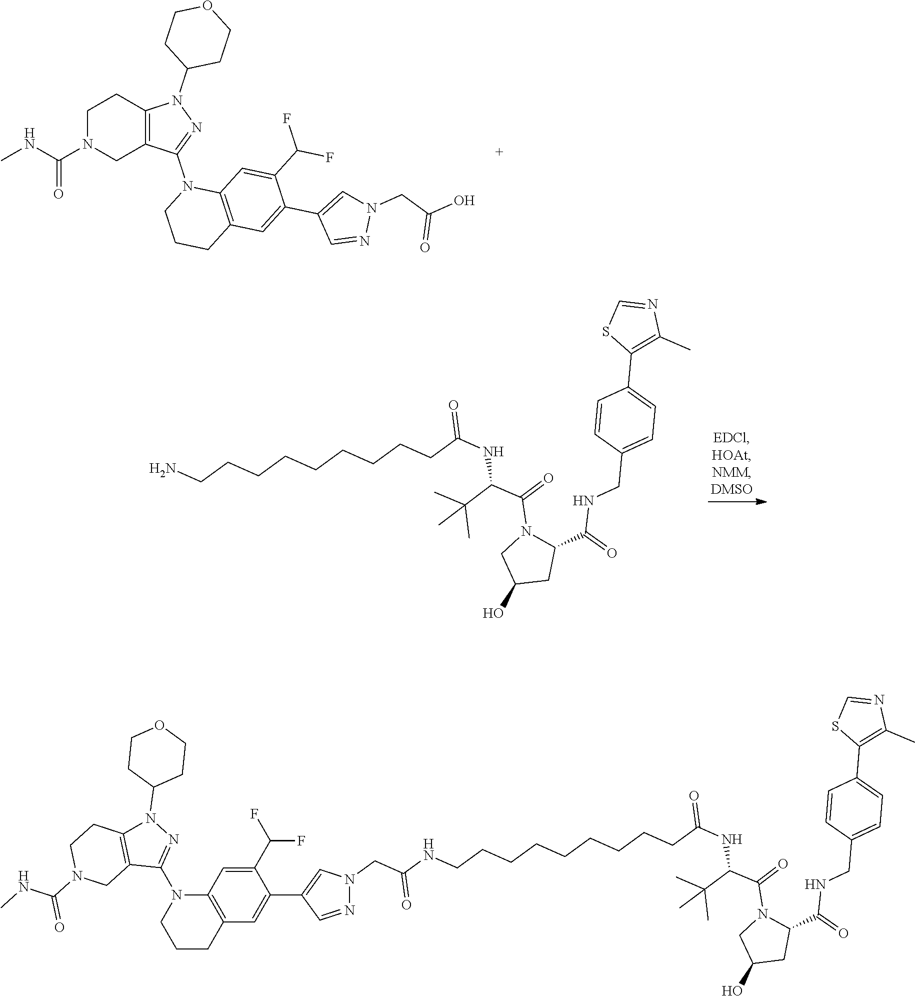

P-049 was synthesized following the standard procedure for preparing P-001 (0.52 mg, yield 3%). MS (ESI) m/z: 1152.2 [M+H]⁺.

### Example 135. 3-(7-(Difluoromethyl)-6-(1-(2-((11-(((S)-1-((2S,4R)-4-hydroxy-2-((4-(4-methylthiazol-5-yl)benzyl)carbamoyl)pyrrolidin-1-yl)-3,3-dimethyl-1-oxobutan-2-yl)amino)-11-oxoundecyl)amino)-2-oxoethyl)-1H-pyrazol-4-yl)-3,4-dihydroquinolin-1(2H)-yl)-N-methyl-1-(tetrahydro-2H-pyran-4-yl)-1,4,6,7-tetrahydro-5H-pyrazolo[4,3-c]pyridine-5-carboxamide (P-050)

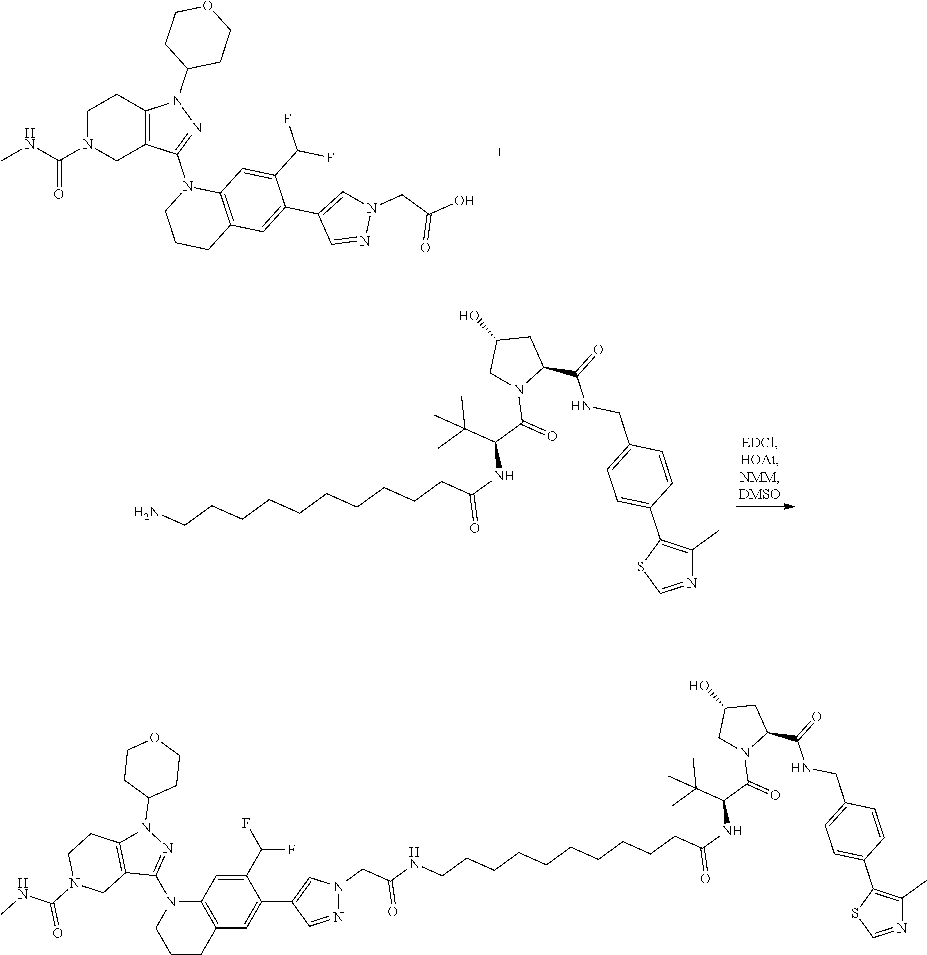

P-050 was synthesized following the standard procedure for preparing P-001 (0.74 mg, yield 4%). MS (ESI) m/z: 1166.3 [M+H]⁺.

### Example 136. 3-(7-(Difluoromethyl)-6-(1-methyl-1H-pyrazol-4-yl)-3,4-dihydroquinolin-1(2H)-yl)-1-(1-(2-(2-(((S)-1-((2S,4R)-4-hydroxy-2-((4-(4-methylthiazol-5-yl)benzyl)carbamoyl)pyrrolidin-1-yl)-3,3-dimethyl-1-oxobutan-2-yl)amino)-2-oxoethoxy)acetyl)piperidin-4-yl)-N-methyl-1,4,6,7-tetrahydro-5H-pyrazolo[4,3-c]pyridine-5-carboxamide (CPD-051/P-064)

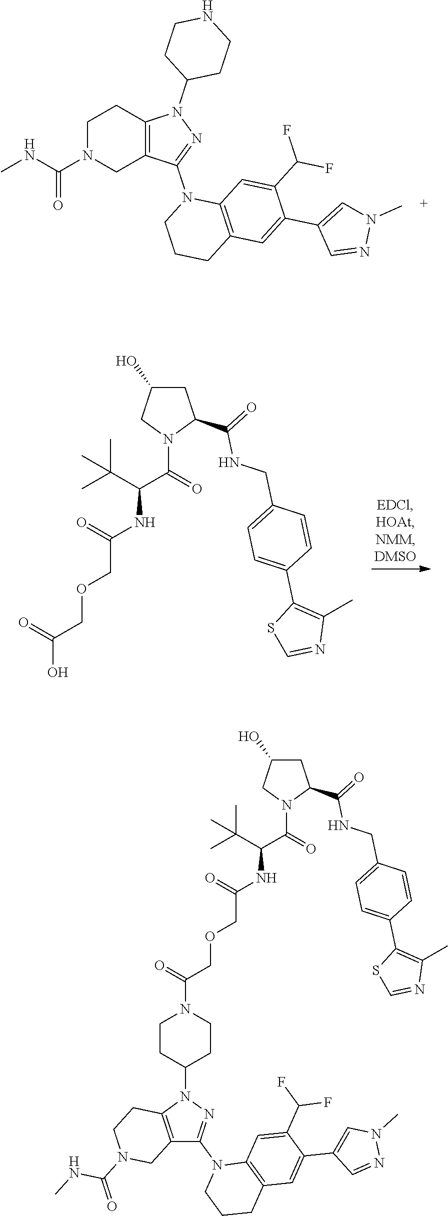

P-064 was synthesized following the standard procedure for preparing P-001 (3.7 mg, yield 26%). MS (ESI) m/z: 1053.9 [M+H]⁺.

### Example 137. 3-(7-(Difluoromethyl)-6-(1-methyl-1H-pyrazol-4-yl)-3,4-dihydroquinolin-1(2H)-yl)-1-(1-(3-(3-(((S)-1-((2S,4R)-4-hydroxy-2-((4-(4-methylthiazol-5-yl)benzyl)carbamoyl)pyrrolidin-1-yl)-3,3-dimethyl-1-oxobutan-2-yl)amino)-3-oxopropoxy)propanoyl)piperidin-4-yl)-N-methyl-1,4,6,7-tetrahydro-5H-pyrazolo[4,3-c]pyridine-5-carboxamide (CPD-052/P-065)

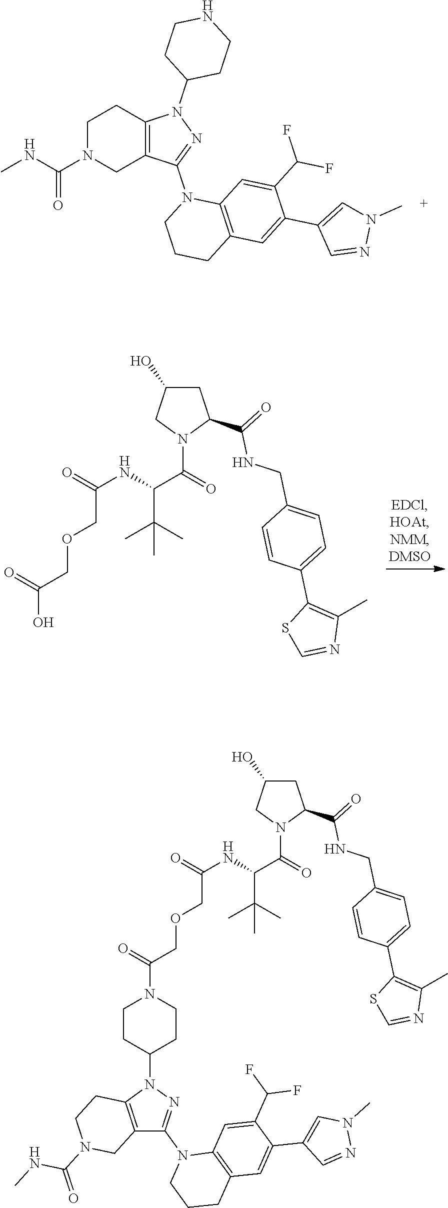

P-065 was synthesized following the standard procedure for preparing P-001 (4.2 mg, yield 29%). MS (ESI) m/z: 1082.0 [M+H]⁺.

### Example 138. 3-(7-(Difluoromethyl)-6-(1-methyl-1H-pyrazol-4-yl)-3,4-dihydroquinolin-1(2H)-yl)-1-(1-(2-(2-(2-(((S)-1-((2S,4R)-4-hydroxy-2-((4-(4-methylthiazol-5-yl)benzyl)carbamoyl)pyrrolidin-1-yl)-3,3-dimethyl-1-oxobutan-2-yl)amino)-2-oxoethoxy)ethoxy)acetyl)piperidin-4-yl)-N-methyl-1,4,6,7-tetrahydro-5H-pyrazolo[4,3-c]pyridine-5-carboxamide (CPD-053/P-066)

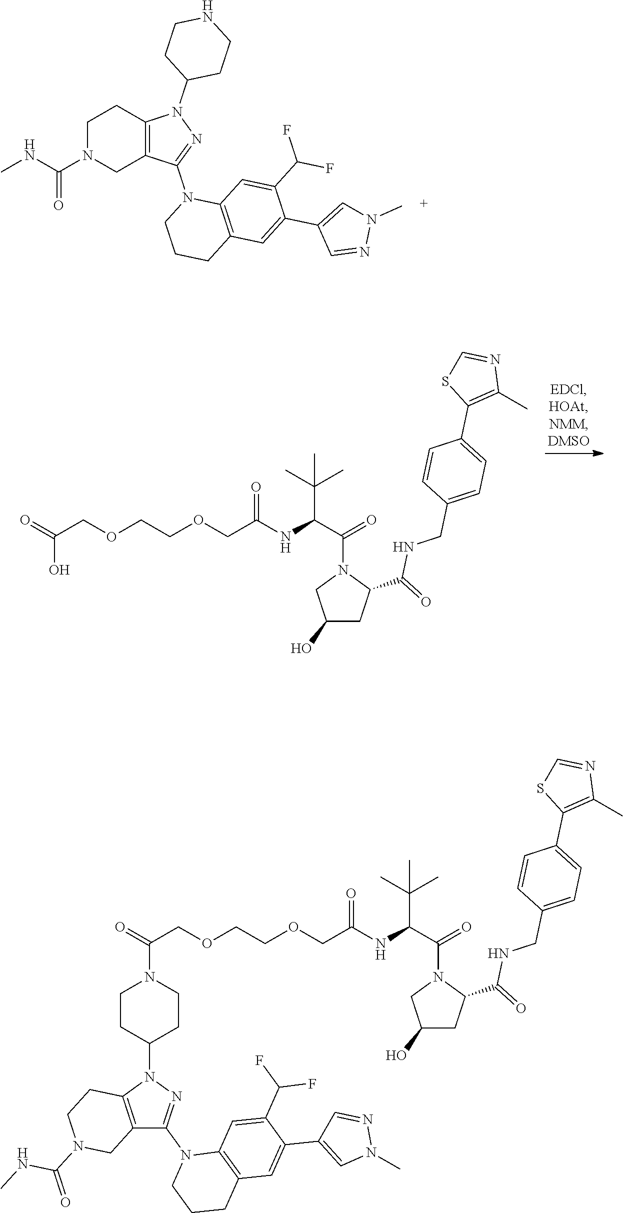

P-066 was synthesized following the standard procedure for preparing P-001 (4.3 mg, yield 29%). MS (ESI) m/z: 1097.9 [M+H]⁺.

### Example 139. 3-(7-(Difluoromethyl)-6-(1-methyl-1H-pyrazol-4-yl)-3,4-dihydroquinolin-1(2H)-yl)-1-(1-(3-(2-(3-(((S)-1-((2S,4R)-4-hydroxy-2-((4-(4-methylthiazol-5-yl)benzyl)carbamoyl)pyrrolidin-1-yl)-3,3-dimethyl-1-oxobutan-2-yl)amino)-3-oxopropoxy)ethoxy)propanoyl)piperidin-4-yl)-N-methyl-1,4,6,7-tetrahydro-5H-pyrazolo[4,3-c]pyridine-5-carboxamide (CPD-054/P-067)

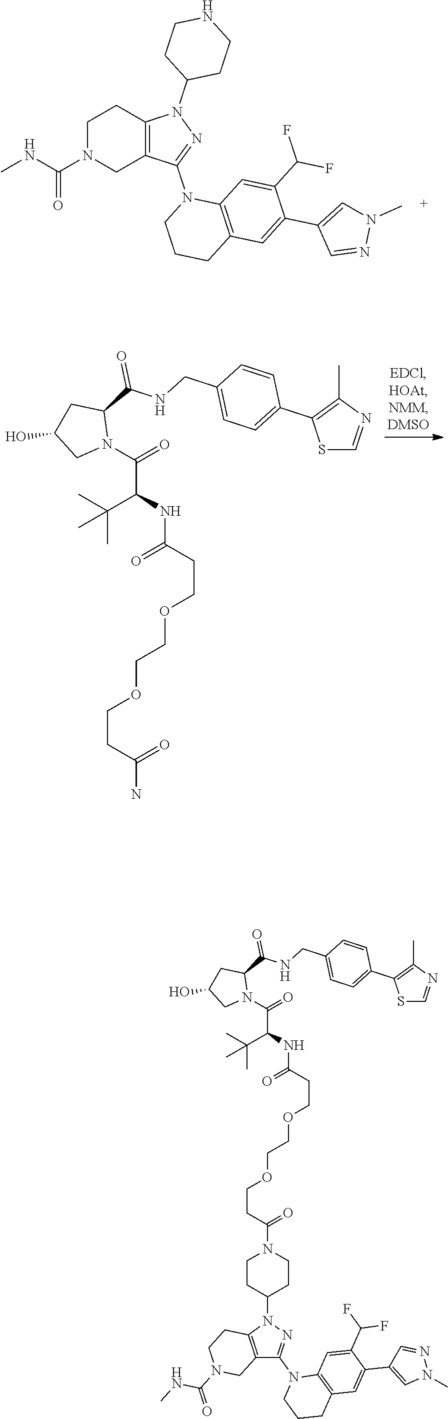

P-067 was synthesized following the standard procedure for preparing P-001 (5.2 mg, yield 35%). MS (ESI) m/z: 1126.0 [M+H]⁺.

### Example 140. 3-(7-(Difluoromethyl)-6-(1-methyl-1H-pyrazol-4-yl)-3,4-dihydroquinolin-1(2H)-yl)-1-(1-((S)-13-((2S,4R)-4-hydroxy-2-((4-(4-methylthiazol-5-yl)benzyl)carbamoyl)pyrrolidine-1-carbonyl)-14,14-dimethyl-11-oxo-3,6,9-trioxa-12-azapentadecanoyl)piperidin-4-yl)-N-methyl-1,4,6,7-tetrahydro-5H-pyrazolo[4,3-c]pyridine-5-carboxamide (CPD-055/P-068)

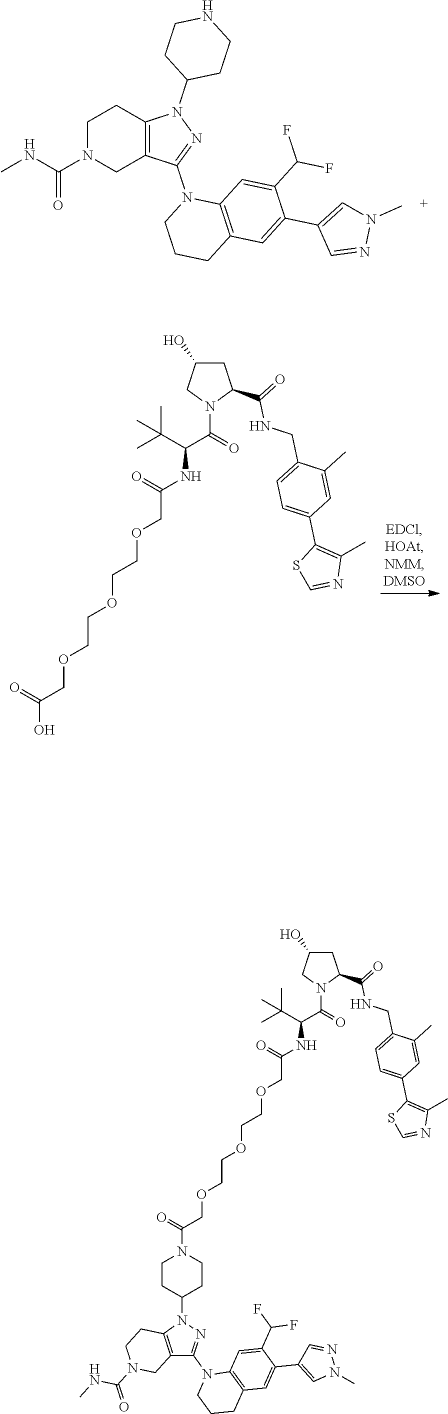

P-068 was synthesized following the standard procedure for preparing P-001 (5.6 mg, yield 37%). MS (ESI) m/z: 1142.0 [M+H]⁺.

### Example 141. 3-(7-(Difluoromethyl)-6-(1-methyl-1H-pyrazol-4-yl)-3,4-dihydroquinolin-1(2H)-yl)-1-(1-((S)-15-((2S,4R)-4-hydroxy-2-((4-(4-methylthiazol-5-yl)benzyl)carbamoyl)pyrrolidine-1-carbonyl)-16,16-dimethyl-13-oxo-4,7,10-trioxa-14-azaheptadecanoyl)piperidin-4-yl)-N-methyl-1,4,6,7-tetrahydro-5H-pyrazolo[4,3-c]pyridine-5-carboxamide (CPD-056/P-069)

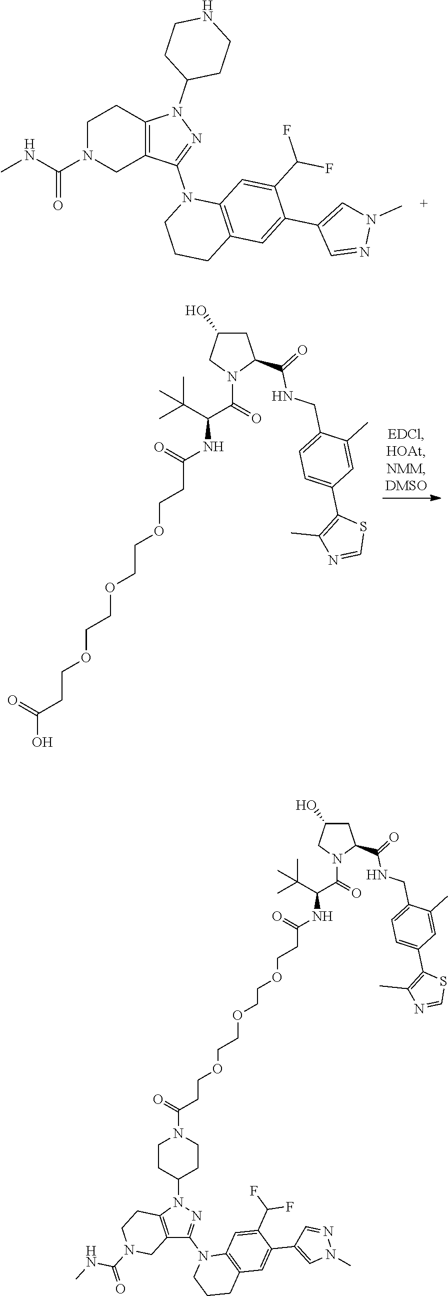

P-069 was synthesized following the standard procedure for preparing P-001 (6.2 mg, yield 40%). MS (ESI) m/z: 1170.1 [M+H]⁺.

### Example 142. 3-(7-(Difluoromethyl)-6-(1-methyl-1H-pyrazol-4-yl)-3,4-dihydroquinolin-1(2H)-yl)-1-(1-((S)-18-((2S,4R)-4-hydroxy-2-((4-(4-methylthiazol-5-yl)benzyl)carbamoyl)pyrrolidine-1-carbonyl)-19,19-dimethyl-16-oxo-4,7,10,13-tetraoxa-17-azaicosanoyl)piperidin-4-yl)-N-methyl-1,4,6,7-tetrahydro-5H-pyrazolo[4,3-c]pyridine-5-carboxamide (CPD-057/P-070)

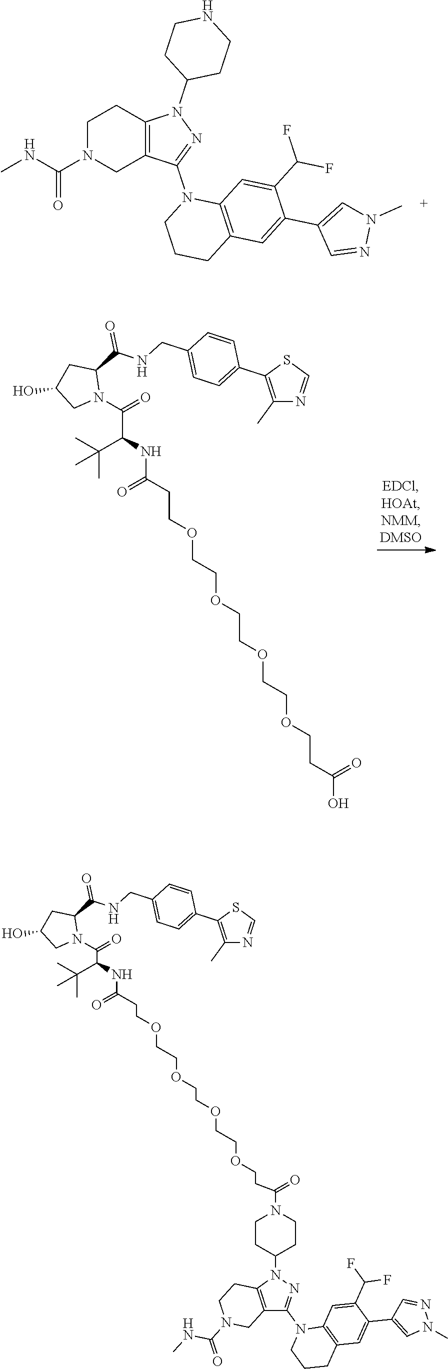

P-070 was synthesized following the standard procedure for preparing P-001 (4.2 mg, yield 26%). MS (ESI) m/z: 1214.1 [M+H]⁺.

### Example 143. 3-(7-(Difluoromethyl)-6-(1-methyl-1H-pyrazol-4-yl)-3,4-dihydroquinolin-1(2H)-yl)-1-(1-((S)-19-((2S,4R)-4-hydroxy-2-((4-(4-methylthiazol-5-yl)benzyl)carbamoyl)pyrrolidine-1-carbonyl)-20,20-dimethyl-17-oxo-3,6,9,12,15-pentaoxa-18-azahenicosanoyl)piperidin-4-yl)-N-methyl-1,4,6,7-tetrahydro-5H-pyrazolo[4,3-c]pyridine-5-carboxamide (CPD-058/P-071)

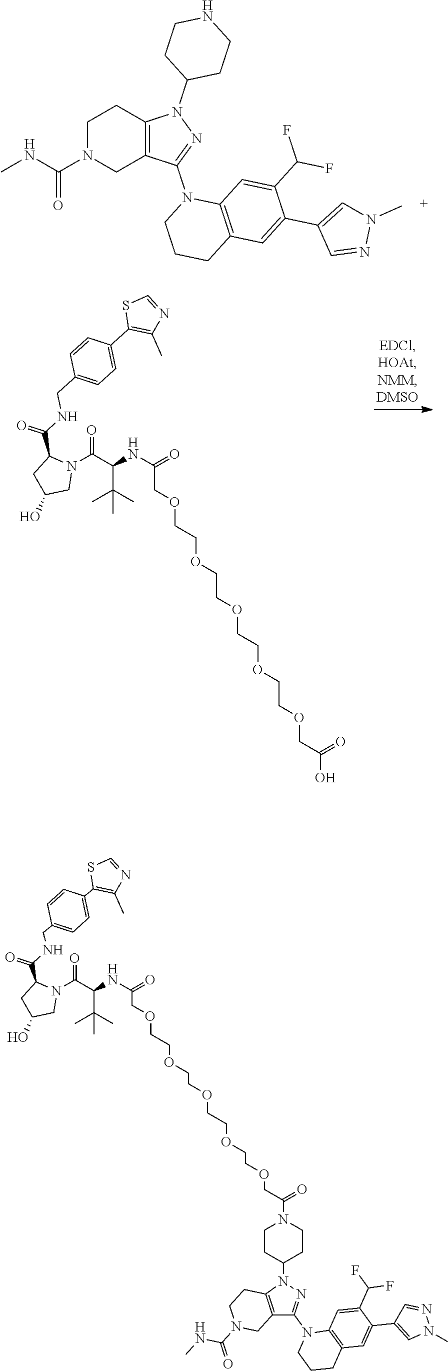

P-071 was synthesized following the standard procedure for preparing P-001 (5.4 mg, yield 33%). MS (ESI) m/z: 1230.0 [M+H]⁺.

### Example 144. 3-(7-(Difluoromethyl)-6-(1-methyl-1H-pyrazol-4-yl)-3,4-dihydroquinolin-1(2H)-yl)-1-(1-((S)-21-((2S,4R)-4-hydroxy-2-((4-(4-methylthiazol-5-yl)benzyl)carbamoyl)pyrrolidine-1-carbonyl)-22,22-dimethyl-19-oxo-4,7,10,13,16-pentaoxa-20-azatricosanoyl)piperidin-4-yl)-N-methyl-1,4,6,7-tetrahydro-5H-pyrazolo[4,3-c]pyridine-5-carboxamide (CPD-059/P-072)

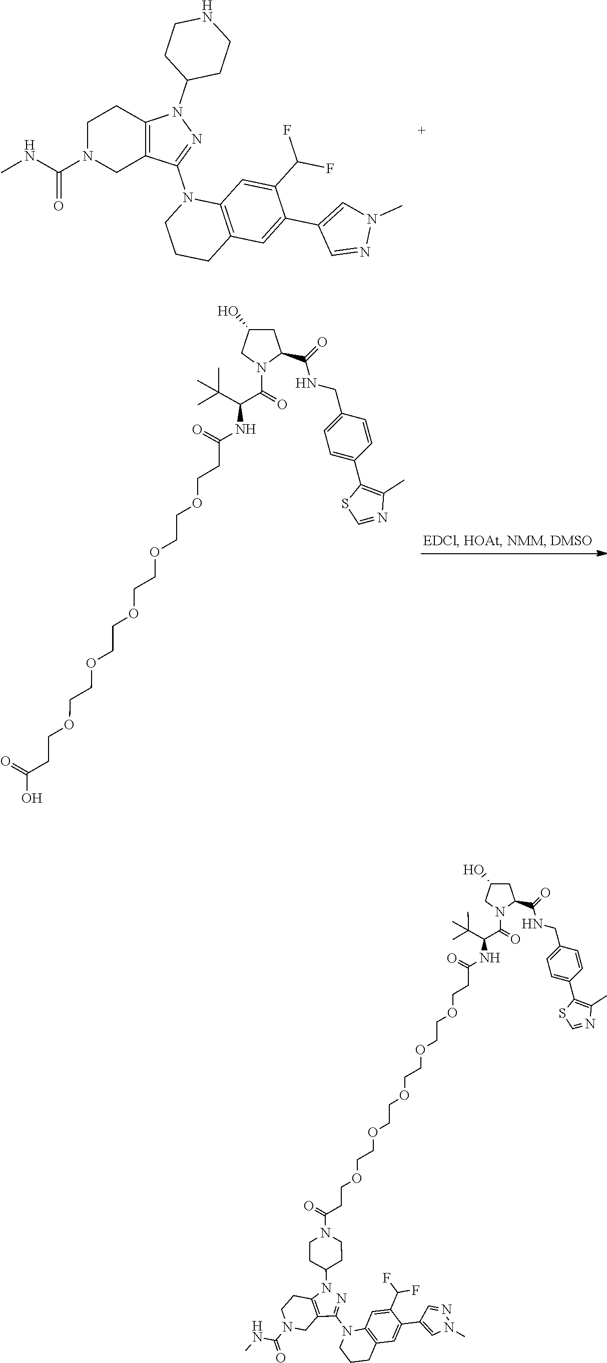

P-072 was synthesized following the standard procedure for preparing P-001 (5.1 mg, yield 30%). MS (ESI) m/z: 1258.2 [M+H]⁺.

### Example 145. 3-(7-(Difluoromethyl)-6-(1-methyl-1H-pyrazol-4-yl)-3,4-dihydroquinolin-1(2H)-yl)-1-(1-(4-(((S)-1-((2S,4R)-4-hydroxy-2-((4-(4-methylthiazol-5-yl)benzyl)carbamoyl)pyrrolidin-1-yl)-3,3-dimethyl-1-oxobutan-2-yl)amino)-4-oxobutanoyl)piperidin-4-yl)-N-methyl-1,4,6,7-tetrahydro-5H-pyrazolo[4,3-c]pyridine-5-carboxamide (CPD-060/P-056)

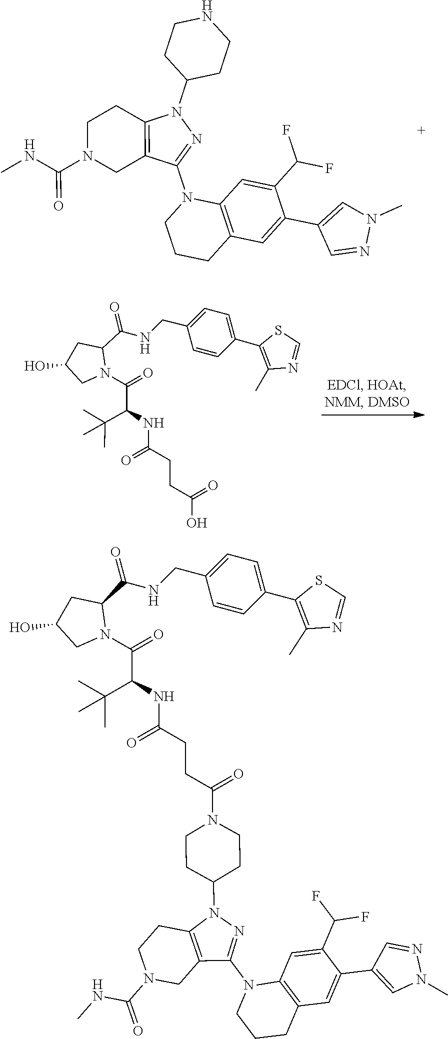

P-056 was synthesized following the standard procedure for preparing P-001 (4.5 mg, yield 33%). MS (ESI) m/z: 1038.0 [M+H]⁺.

### Example 146. 3-(7-(Difluoromethyl)-6-(1-methyl-1H-pyrazol-4-yl)-3,4-dihydroquinolin-1(2H)-yl)-1-(1-(5-(((S)-1-((2S,4R)-4-hydroxy-2-((4-(4-methylthiazol-5-yl)benzyl)carbamoyl)pyrrolidin-1-yl)-3,3-dimethyl-1-oxobutan-2-yl)amino)-5-oxopentanoyl)piperidin-4-yl)-N-methyl-1,4,6,7-tetrahydro-5H-pyrazolo[4,3-c]pyridine-5-carboxamide (CPD-061/P-057)

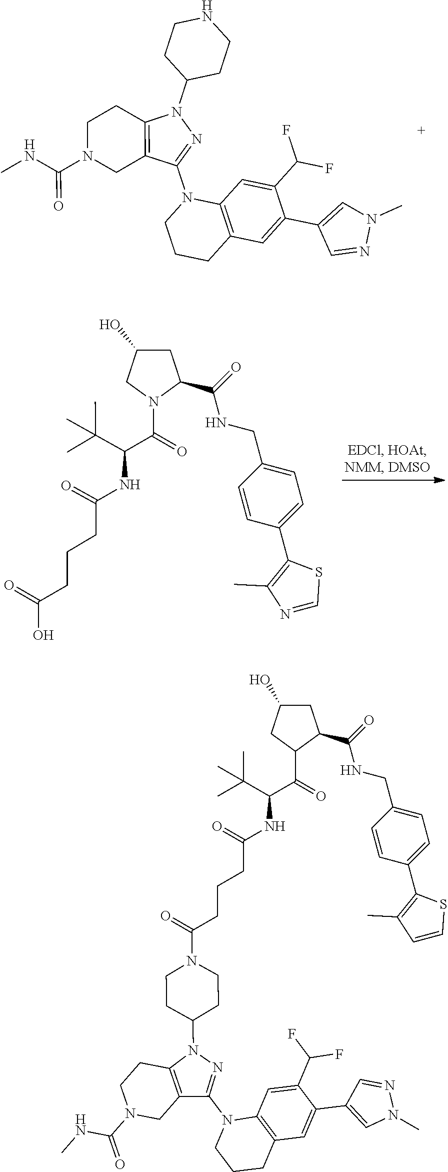

P-057 was synthesized following the standard procedure for preparing P-001 (5.0 mg, yield 36%). MS (ESI) m/z: 1052.1 [M+H]⁺.

### Example 147. 3-(7-(Difluoromethyl)-6-(1-methyl-1H-pyrazol-4-yl)-3,4-dihydroquinolin-1(2H)-yl)-1-(1-(6-(((S)-1-((2S,4R)-4-hydroxy-2-((4-(4-methylthiazol-5-yl)benzyl)carbamoyl)pyrrolidin-1-yl)-3,3-dimethyl-1-oxobutan-2-yl)amino)-6-oxohexanoyl)piperidin-4-yl)-N-methyl-1,4,6,7-tetrahydro-5H-pyrazolo[4,3-c]pyridine-5-carboxamide (CPD-062/P-058)

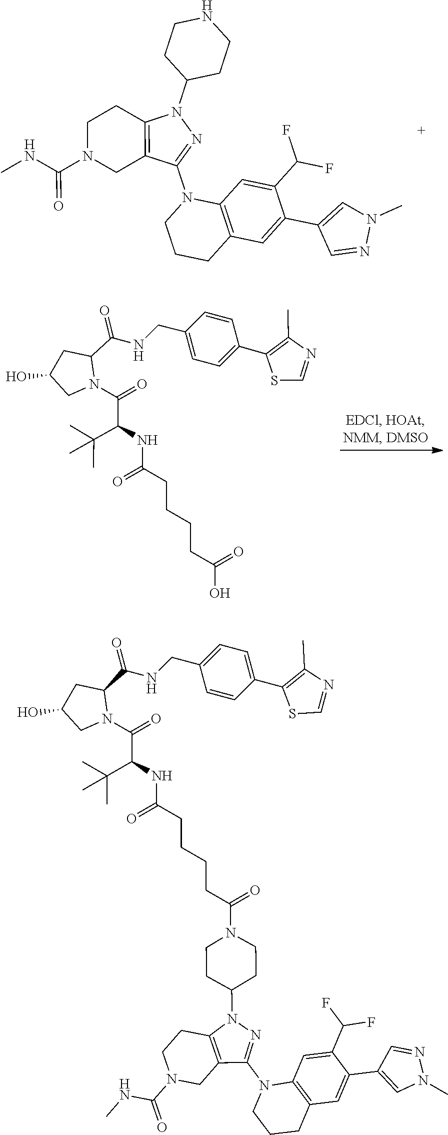

P-058 was synthesized following the standard procedure for preparing P-001 (3.7 mg, yield 26%). MS (ESI) m/z: 1066.0 [M+H]⁺.

### Example 148. 3-(7-(Difluoromethyl)-6-(1-methyl-1H-pyrazol-4-yl)-3,4-dihydroquinolin-1(2H)-yl)-1-(1-(7-(((S)-1-((2S,4R)-4-hydroxy-2-((4-(4-methylthiazol-5-yl)benzyl)carbamoyl)pyrrolidin-1-yl)-3,3-dimethyl-1-oxobutan-2-yl)amino)-7-oxoheptanoyl)piperidin-4-yl)-N-methyl-1,4,6,7-tetrahydro-5H-pyrazolo[4,3-c]pyridine-5-carboxamide (CPD-063/P-059)

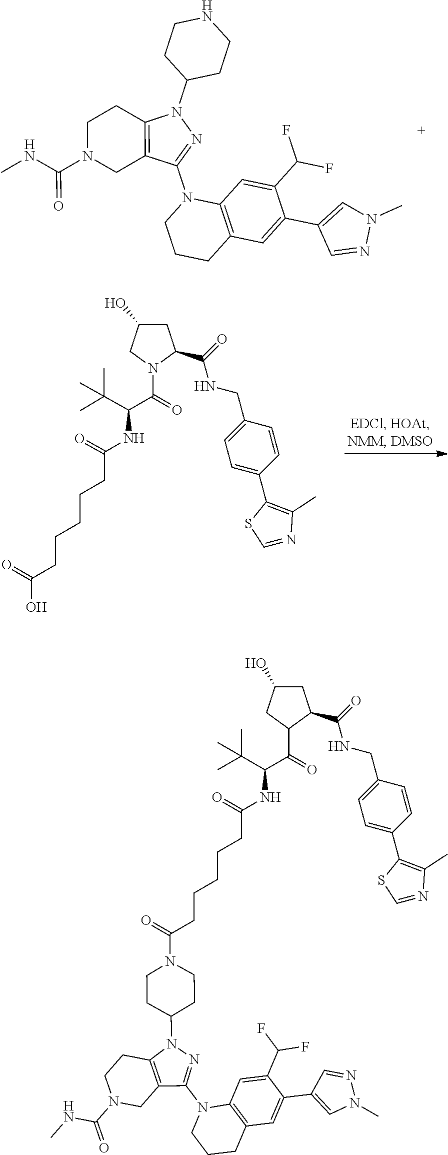

P-059 was synthesized following the standard procedure for preparing P-001 (2.3 mg, yield 16%). MS (ESI) m/z: 1080.0 [M+H]⁺.

### Example 149. 3-(7-(Difluoromethyl)-6-(1-methyl-1H-pyrazol-4-yl)-3,4-dihydroquinolin-1(2H)-yl)-1-(1-(8-(((S)-1-((2S,4R)-4-hydroxy-2-((4-(4-methylthiazol-5-yl)benzyl)carbamoyl)pyrrolidin-1-yl)-3,3-dimethyl-1-oxobutan-2-yl)amino)-8-oxooctanoyl)piperidin-4-yl)-N-methyl-1,4,6,7-tetrahydro-5H-pyrazolo[4,3-c]pyridine-5-carboxamide (CPD-064/P-060)

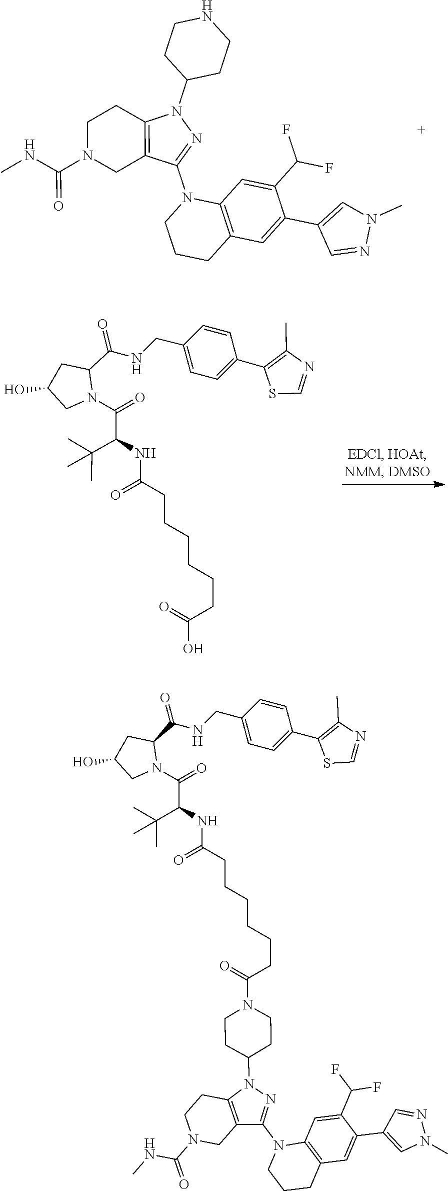

P-060 was synthesized following the standard procedure for preparing P-001 (5.6 mg, yield 38%). MS (ESI) m/z: 1094.0 [M+H]⁺.

### Example 150. 3-(7-(Difluoromethyl)-6-(1-methyl-1H-pyrazol-4-yl)-3,4-dihydroquinolin-1(2H)-yl)-1-(1-(9-(((S)-1-((2S,4R)-4-hydroxy-2-((4-(4-methylthiazol-5-yl)benzyl)carbamoyl)pyrrolidin-1-yl)-3,3-dimethyl-1-oxobutan-2-yl)amino)-9-oxononanoyl)piperidin-4-yl)-N-methyl-1,4,6,7-tetrahydro-5H-pyrazolo[4,3-c]pyridine-5-carboxamide (CPD-065/P-061)

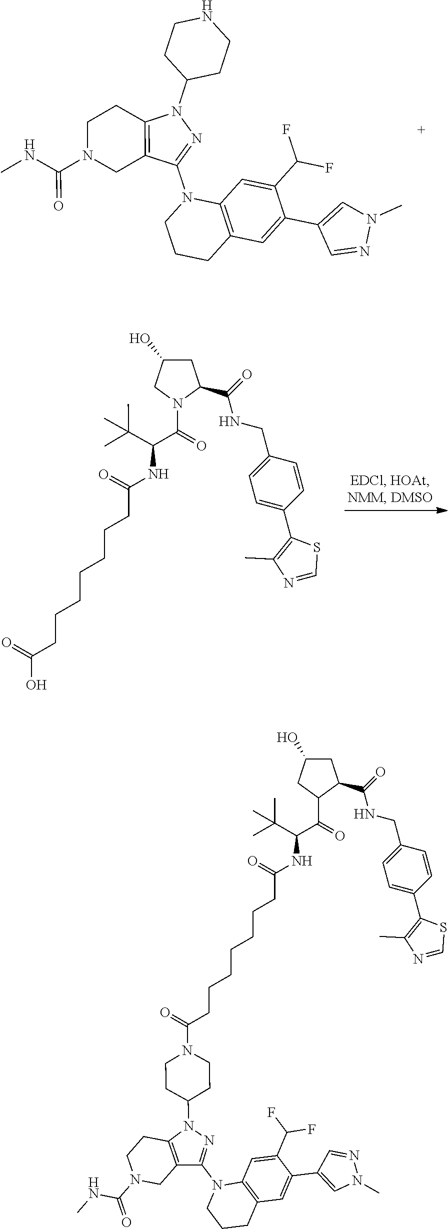

P-061 was synthesized following the standard procedure for preparing P-001 (4.5 mg, yield 30%). MS (ESI) m/z: 1108.1 [M+H]⁺.

### Example 151. 3-(7-(Difluoromethyl)-6-(1-methyl-1H-pyrazol-4-yl)-3,4-dihydroquinolin-1(2H)-yl)-1-(1-(10-(((S)-1-((2S,4R)-4-hydroxy-2-((4-(4-methylthiazol-5-yl)benzyl)carbamoyl)pyrrolidin-1-yl)-3,3-dimethyl-1-oxobutan-2-yl)amino)-10-oxodecanoyl)piperidin-4-yl)-N-methyl-1,4,6,7-tetrahydro-5H-pyrazolo[4,3-c]pyridine-5-carboxamide (CPD-066/P-062)

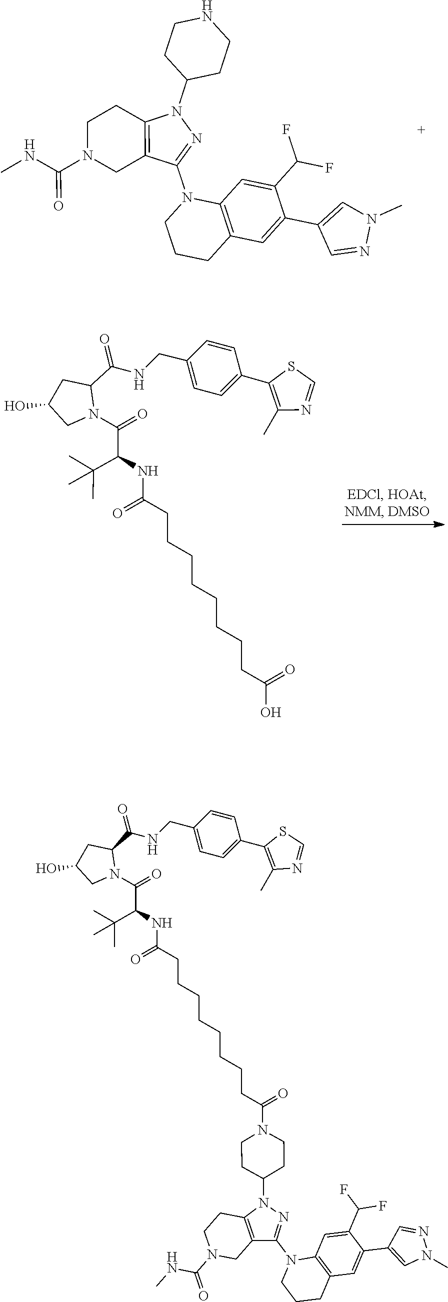

P-062 was synthesized following the standard procedure for preparing P-001 (3.5 mg, yield 23%). MS (ESI) m/z: 1122.2 [M+H]⁺.

### Example 152. 3-(7-(Difluoromethyl)-6-(1-methyl-1H-pyrazol-4-yl)-3,4-dihydroquinolin-1(2H)-yl)-1-(1-(11-(((S)-1-((2S,4R)-4-hydroxy-2-((4-(4-methylthiazol-5-yl)benzyl)carbamoyl)pyrrolidin-1-yl)-3,3-dimethyl-1-oxobutan-2-yl)amino)-11-oxoundecanoyl)piperidin-4-yl)-N-methyl-1,4,6,7-tetrahydro-5H-pyrazolo[4,3-c]pyridine-5-carboxamide (CPD-067/P-063)

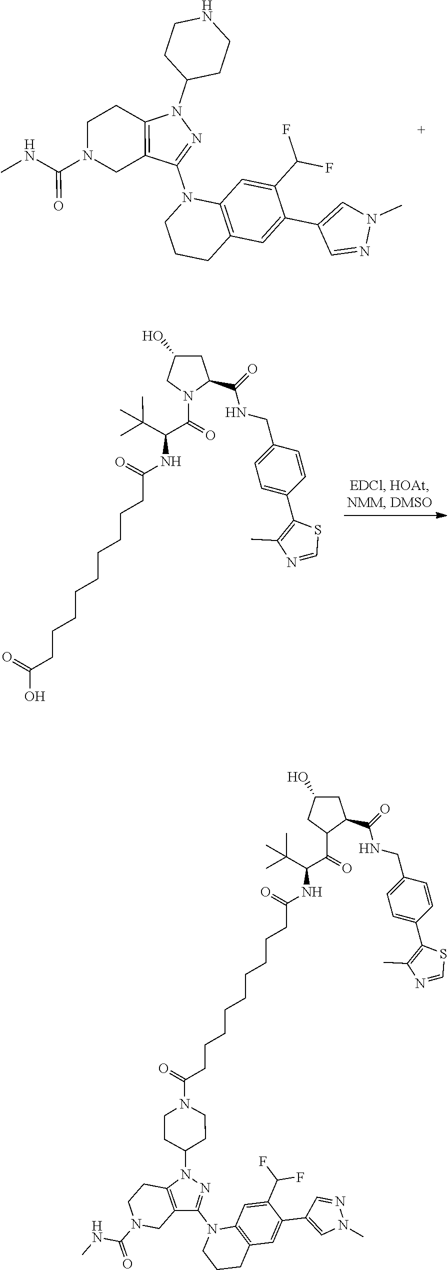

P-063 was synthesized following the standard procedure for preparing P-001 (3.4 mg, yield 22%). MS (ESI) m/z: 1136.1 [M+H]⁺.

### Example 153. 3-(7-(Difluoromethyl)-6-(1-methyl-1H-pyrazol-4-yl)-3,4-dihydroquinolin-1(2H)-yl)-1-(1-(3-(2-((2-(2,6-dioxopiperidin-3-yl)-1,3-dioxoisoindolin-4-yl)amino)ethoxy)propanoyl)piperidin-4-yl)-N-methyl-1,4,6,7-tetrahydro-5H-pyrazolo[4,3-c]pyridine-5-carboxamide (CPD-068/P-051)

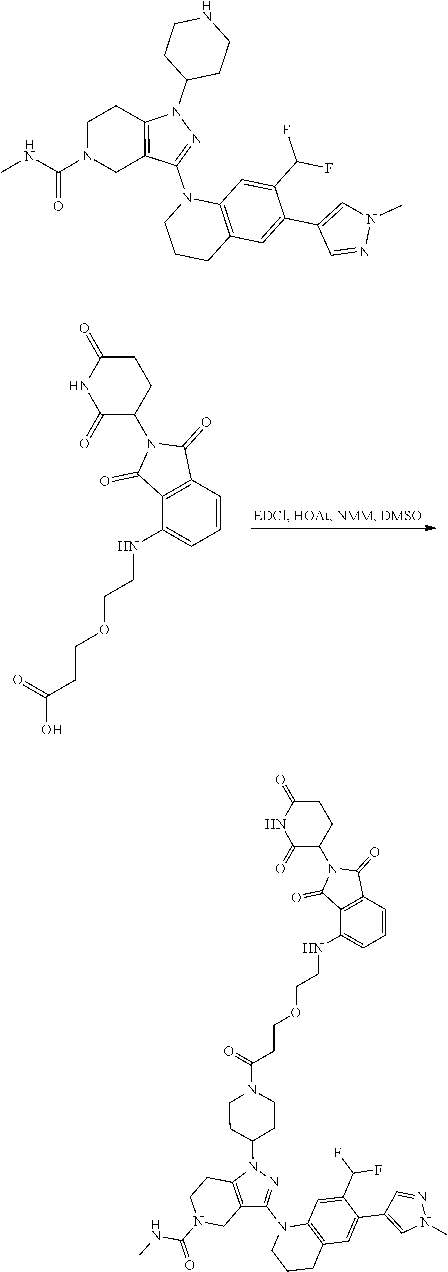

P-051 was synthesized following the standard procedure for preparing P-001 (3.9 mg, yield 33%). MS (ESI) m/z: 896.9 [M+H]⁺.

### Example 154. 3-(7-(Difluoromethyl)-6-(1-methyl-1H-pyrazol-4-yl)-3,4-dihydroquinolin-1(2H)-yl)-1-(1-(3-(2-(2-((2-(2,6-dioxopiperidin-3-yl)-1,3-dioxoisoindolin-4-yl)amino)ethoxy)ethoxy)propanoyl)piperidin-4-yl)-N-methyl-1,4,6,7-tetrahydro-5H-pyrazolo[4,3-c]pyridine-5-carboxamide (CPD-069/P-052)

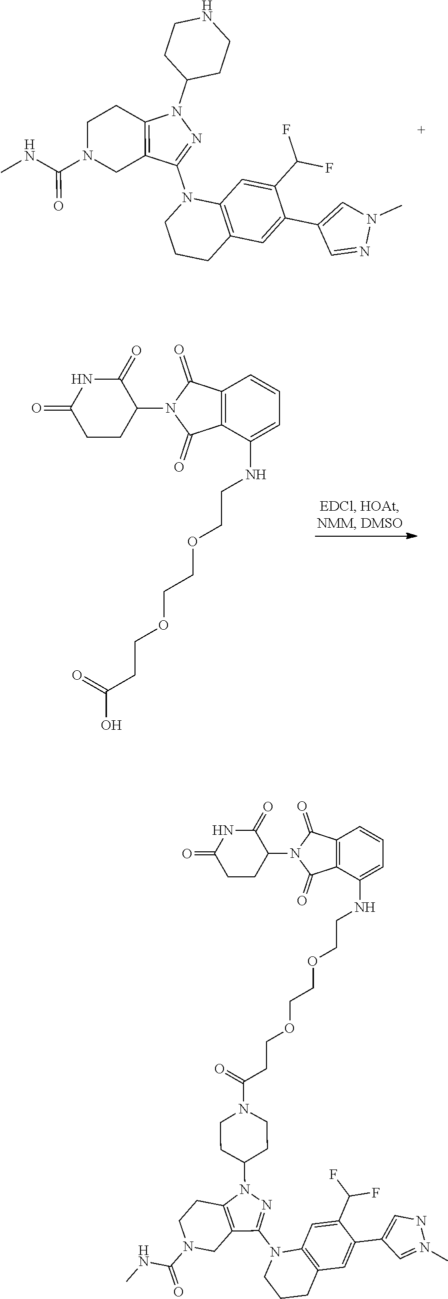

P-052 was synthesized following the standard procedure for preparing P-001 (4.6 mg, yield 37%). MS (ESI) m/z: 940.9 [M+H]⁺.

### Example 155. 3-(7-(Difluoromethyl)-6-(1-methyl-1H-pyrazol-4-yl)-3,4-dihydroquinolin-1(2H)-yl)-1-(1-(3-(2-(2-(2-((2-(2,6-dioxopiperidin-3-yl)-1,3-dioxoisoindolin-4-yl)amino)ethoxy)ethoxy)ethoxy)propanoyl)piperidin-4-yl)-N-methyl-1,4,6,7-tetrahydro-5H-pyrazolo[4,3-c]pyridine-5-carboxamide (CPD-070/P-053)

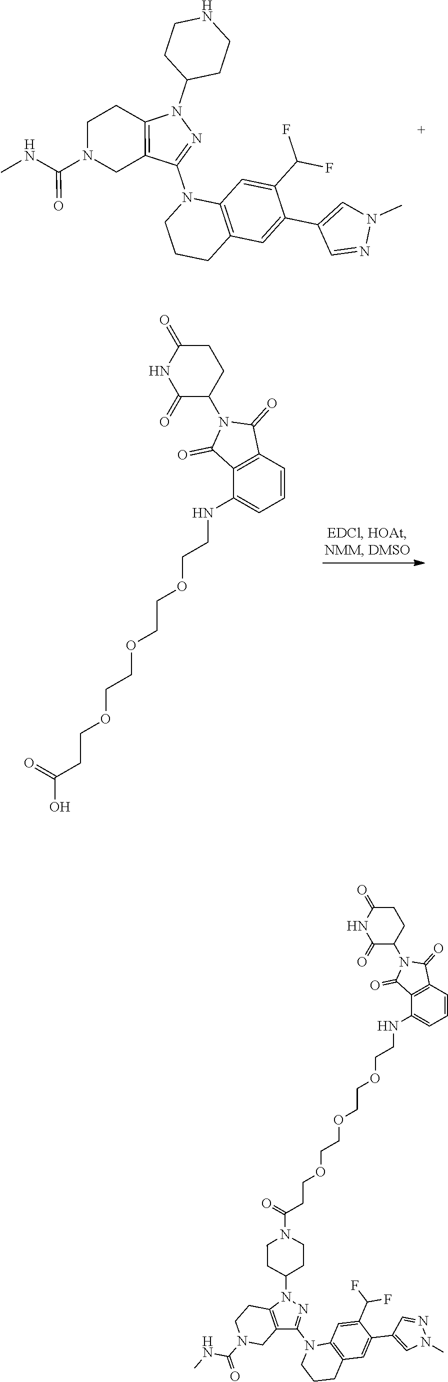

P-053 was synthesized following the standard procedure for preparing P-001 (5.4 mg, yield 41%). MS (ESI) m/z: 985.0 [M+H]⁺.

### Example 156. 3-(7-(Difluoromethyl)-6-(1-methyl-1H-pyrazol-4-yl)-3,4-dihydroquinolin-1(2H)-yl)-1-(1-(1-((2-(2,6-dioxopiperidin-3-yl)-1,3-dioxoisoindolin-4-yl)amino)-3,6,9,12-tetraoxapentadecan-15-oyl)piperidin-4-yl)-N-methyl-1,4,6,7-tetrahydro-5H-pyrazolo[4,3-c]pyridine-5-carboxamide (CPD-071/P-054)

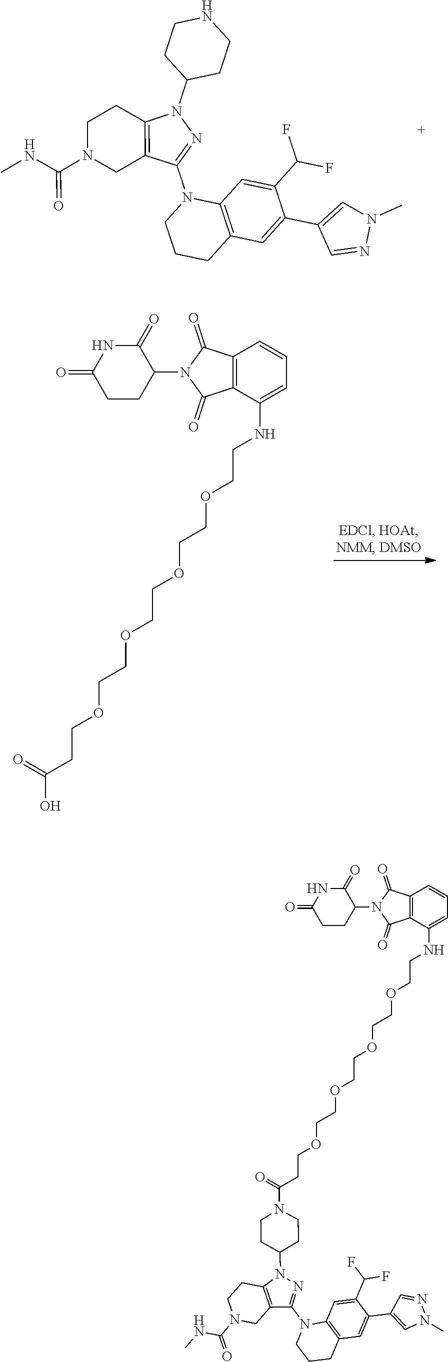

P-054 was synthesized following the standard procedure for preparing P-001 (5.7 mg, yield 42%). MS (ESI) m/z: 1029.0 [M+H]⁺.

### Example 157. 3-(7-(Difluoromethyl)-6-(1-methyl-1H-pyrazol-4-yl)-3,4-dihydroquinolin-1(2H)-yl)-1-(1-(1-((2-(2,6-dioxopiperidin-3-yl)-1,3-dioxoisoindolin-4-yl)amino)-3,6,9,12,15-pentaoxaoctadecan-18-oyl)piperidin-4-yl)-N-methyl-1,4,6,7-tetrahydro-5H-pyrazolo[4,3-c]pyridine-5-carboxamide (CPD-072/P-055)

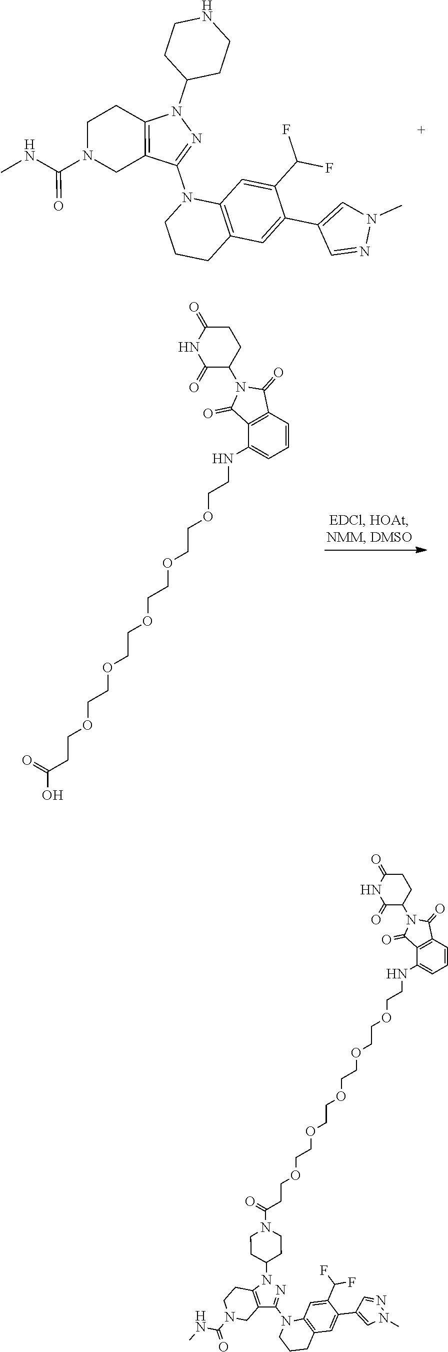

P-055 was synthesized following the standard procedure for preparing P-001 (5.3 mg, yield 37%). MS (ESI) m/z: 1073.1 [M+H]⁺.

### Example 158. 3-(7-(Difluoromethyl)-6-(1-(2-((2-(2-(((S)-1-((2S,4R)-4-hydroxy-2-((4-(4-methylthiazol-5-yl)benzyl)carbamoyl)pyrrolidin-1-yl)-3,3-dimethyl-1-oxobutan-2-yl)amino)-2-oxoethoxy)ethyl)amino)-2-oxoethyl)-1H-pyrazol-4-yl)-3,4-dihydroquinolin-1(2H)-yl)-N-methyl-1-(tetrahydro-2H-pyran-4-yl)-1,4,6,7-tetrahydro-5H-pyrazolo[4,3-c]pyridine-5-carboxamide (P-073)

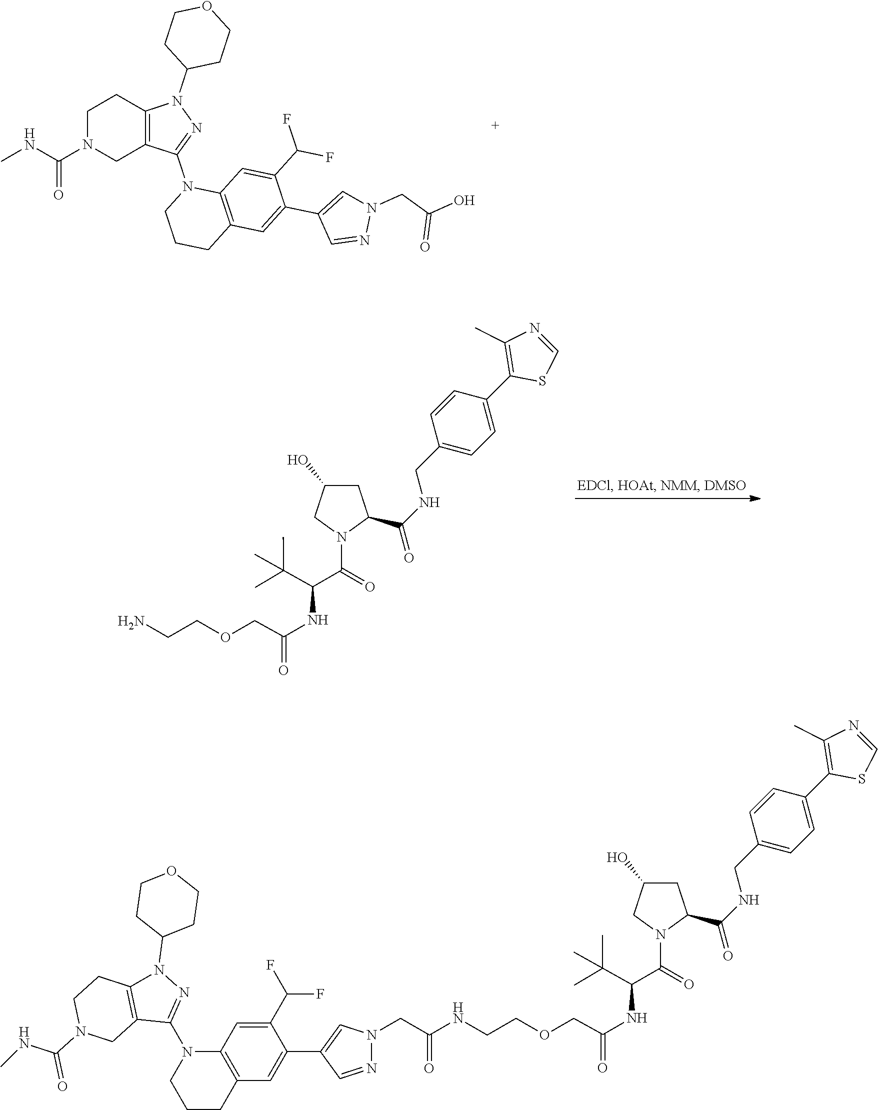 [SMILES: CNC(=O)N1CCc2c(c(N3CCCc4cc(-c5cnn(CC(=O)O)c5)c(C(F)F)cc43)nn2C2CCOCC2)C1 ; Cc1ncsc1-c1ccc(CNC(=O)[C@@H]2C[C@@H](O)CN2C(=O)[C@@H](NC(=O)COCCN)C(C)(C)C)cc1 ; CNC(=O)N1CCc2c(c(N3CCCc4cc(-c5cnn(CC(=O)NCCOCC(=O)N[C@H](C(=O)N6C[C@H](O)C[C@H]6C(=O)NCc6ccc(-c7scnc7C)cc6)C(C)(C)C)c5)c(C(F)F)cc43)nn2C2CCOCC2)C1]

P-073 was synthesized following the standard procedure for preparing P-001 (3.8 mg, yield 40%). MS (ESI) m/z: 1084.0 [M+H]⁺.

### Example 159. 3-(7-(Difluoromethyl)-6-(1-(2-((2-(3-(((S)-1-((2S,4R)-4-hydroxy-2-((4-(4-methylthiazol-5-yl)benzyl)carbamoyl)pyrrolidin-1-yl)-3,3-dimethyl-1-oxobutan-2-yl)amino)-3-oxopropoxy)ethyl)amino)-2-oxoethyl)-1H-pyrazol-4-yl)-3,4-dihydroquinolin-1(2H)-yl)-N-methyl-1-(tetrahydro-2H-pyran-4-yl)-1,4,6,7-tetrahydro-5H-pyrazolo[4,3-c]pyridine-5-carboxamide (P-074)

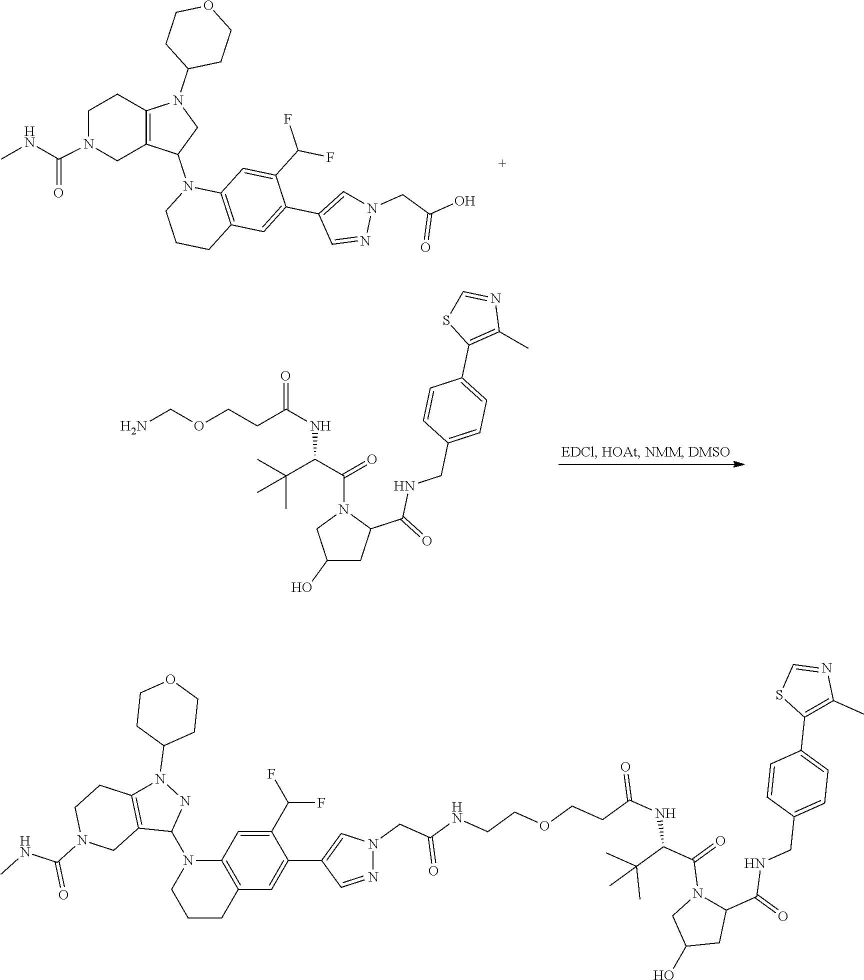

P-074 was synthesized following the standard procedure for preparing P-001 (3.1 mg, yield 32%). MS (ESI) m/z: 1098.0 [M+H]⁺.

### Example 160. 3-(7-(Difluoromethyl)-6-(1-((S)-13-((2S,4R)-4-hydroxy-2-((4-(4-methylthiazol-5-yl)benzyl)carbamoyl)pyrrolidine-1-carbonyl)-14,14-dimethyl-2,11-dioxo-6,9-dioxa-3,12-diazapentadecyl)-1H-pyrazol-4-yl)-3,4-dihydroquinolin-1(2H)-yl)-N-methyl-1-(tetrahydro-2H-pyran-4-yl)-1,4,6,7-tetrahydro-5H-pyrazolo[4,3-c]pyridine-5-carboxamide (CPD-075/P-083)

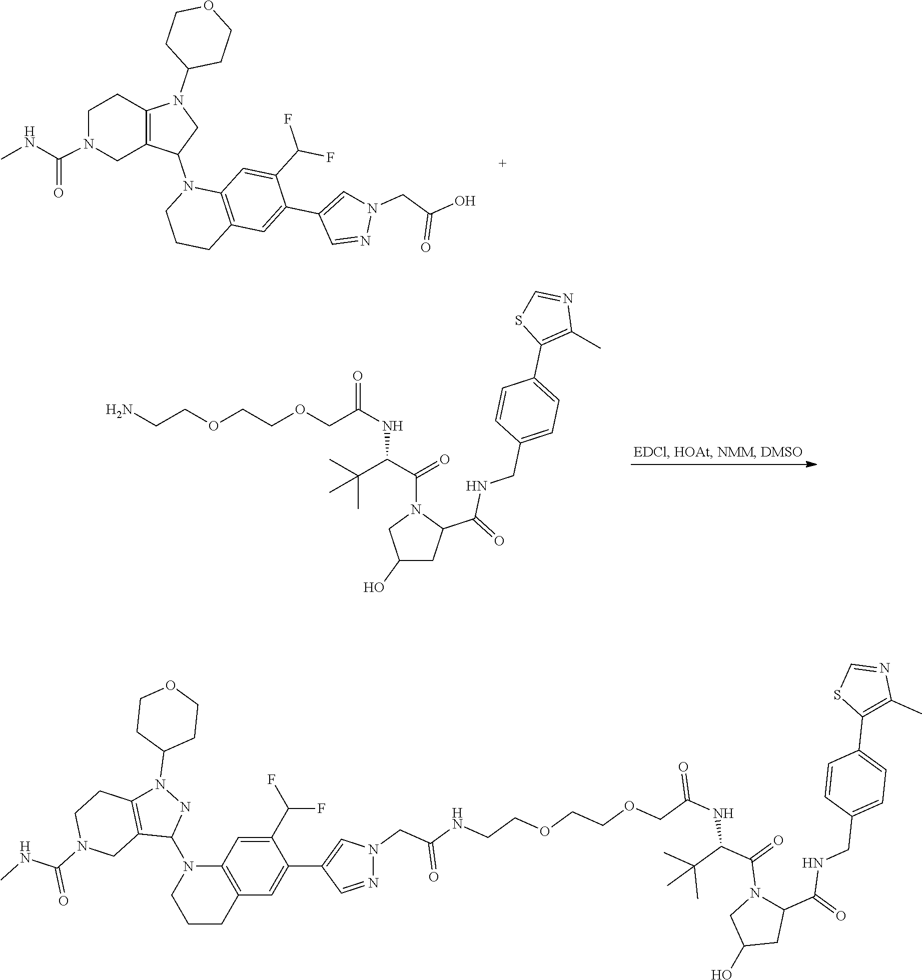

P-083 was synthesized following the standard procedure for preparing P-001 (1.37 mg, yield 14%). MS (ESI) m/z: 1128.1 [M+H]⁺.

### Example 161. 3-(7-(Difluoromethyl)-6-(1-((S)-14-((2S,4R)-4-hydroxy-2-((4-(4-methylthiazol-5-yl)benzyl)carbamoyl)pyrrolidine-1-carbonyl)-15,15-dimethyl-2,12-dioxo-6,9-dioxa-3,13-diazahexadecyl)-1H-pyrazol-4-yl)-3,4-dihydroquinolin-1(2H)-yl)-N-methyl-1-(tetrahydro-2H-pyran-4-yl)-1,4,6,7-tetrahydro-5H-pyrazolo[4,3-c]pyridine-5-carboxamide (CPD-076/P-075)

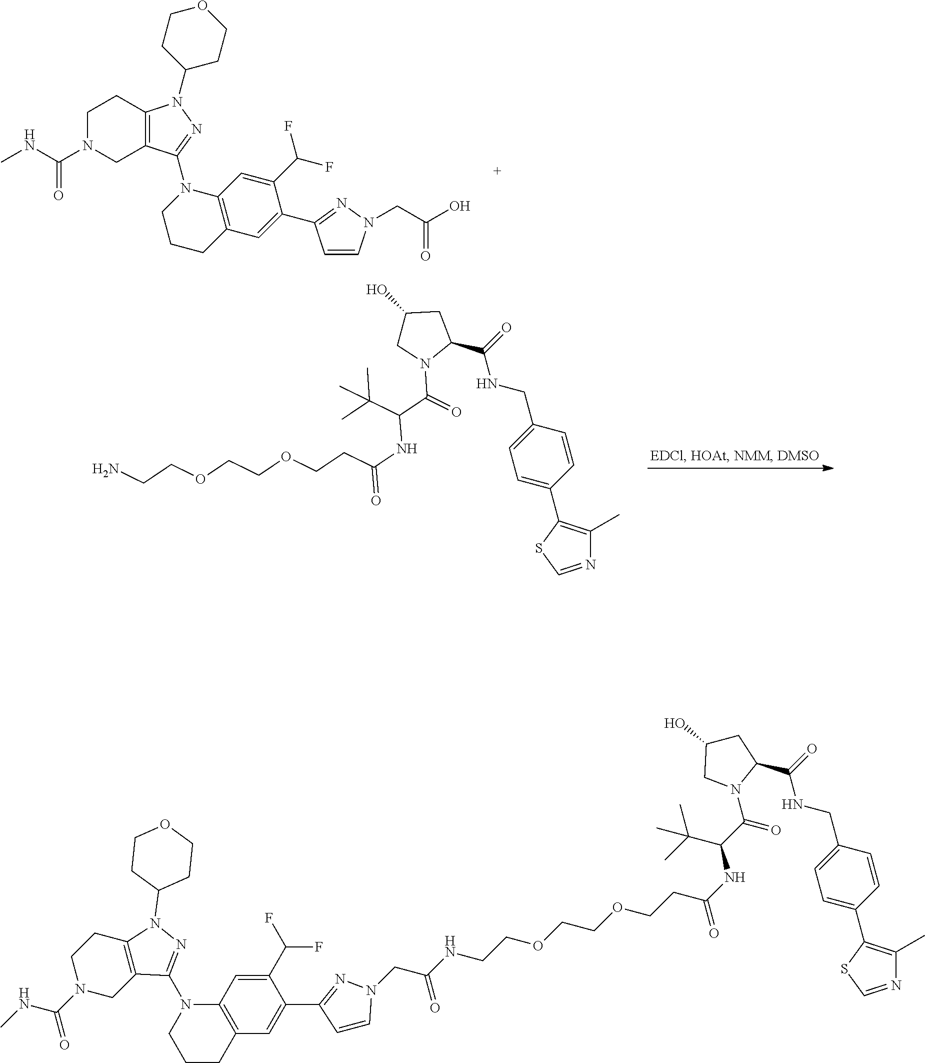

P-075 was synthesized following the standard procedure for preparing P-001 (3.4 mg, yield 34%). MS (ESI) m/z: 1142.1 [M+H]⁺.

### Example 162. 3-(7-(Difluoromethyl)-6-(1-((S)-16-((2S,4R)-4-hydroxy-2-((4-(4-methylthiazol-5-yl)benzyl)carbamoyl)pyrrolidine-1-carbonyl)-17,17-dimethyl-2,14-dioxo-6,9,12-trioxa-3,15-diazaoctadecyl)-1H-pyrazol-4-yl)-3,4-dihydroquinolin-1(2H)-yl)-N-methyl-1-(tetrahydro-2H-pyran-4-yl)-1,4,6,7-tetrahydro-5H-pyrazolo[4,3-c]pyridine-5-carboxamide (CPD-077/P-076)

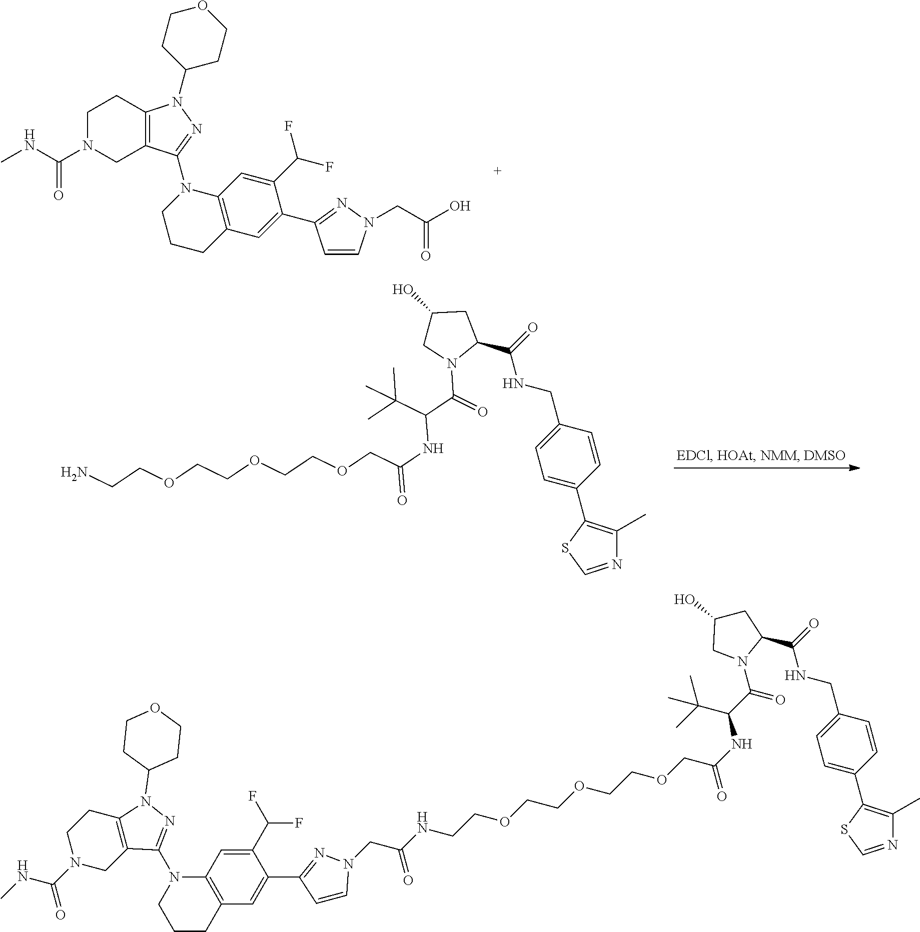

P-076 was synthesized following the standard procedure for preparing P-001 (3.6 mg, yield 35%). MS (ESI) m/z: 1172.1 [M+H]⁺.

### Example 163. 3-(7-(Difluoromethyl)-6-(1-((S)-17-((2S,4R)-4-hydroxy-2-((4-(4-methylthiazol-5-yl)benzyl)carbamoyl)pyrrolidine-1-carbonyl)-18,18-dimethyl-2,15-dioxo-6,9,12-trioxa-3,16-diazanonadecyl)-1H-pyrazol-4-yl)-3,4-dihydroquinolin-1(2H)-yl)-N-methyl-1-(tetrahydro-2H-pyran-4-yl)-1,4,6,7-tetrahydro-5H-pyrazolo[4,3-c]pyridine-5-carboxamide (CPD-078/P-077)

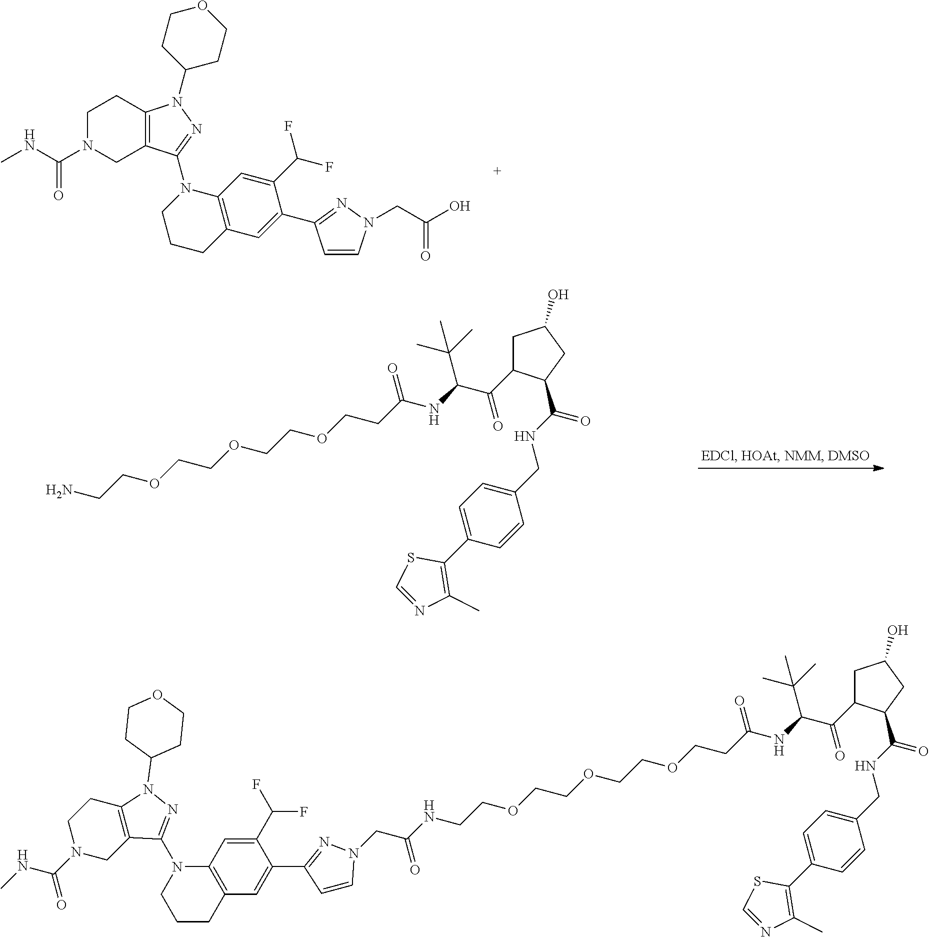 [SMILES: CNC(=O)N1CCc2c(c(N3CCCc4cc(-c5ccn(CC(=O)O)n5)c(C(F)F)cc43)nn2C2CCOCC2)C1 ; Cc1ncsc1-c1ccc(CNC(=O)[C@@H]2C[C@@H](O)CC2C(=O)[C@@H](NC(=O)CCOCCOCCOCCN)C(C)(C)C)cc1 ; CNC(=O)N1CCc2c(c(N3CCCc4cc(-c5ccn(CC(=O)NCCOCCOCCOCCC(=O)N[C@H](C(=O)C6C[C@H](O)C[C@H]6C(=O)NCc6ccc(-c7scnc7C)cc6)C(C)(C)C)n5)c(C(F)F)cc43)nn2C2CCOCC2)C1]

P-077 was synthesized following the standard procedure for preparing P-001 (3.0 mg, yield 29%). MS (ESI) m/z: 1186.3 [M+H]⁺.

### Example 164. 3-(7-(Difluoromethyl)-6-(1-((S)-20-((2S,4R)-4-hydroxy-2-((4-(4-methylthiazol-5-yl)benzyl)carbamoyl)pyrrolidine-1-carbonyl)-21,21-dimethyl-2,18-dioxo-6,9,12,15-tetraoxa-3,19-diazadocosyl)-1H-pyrazol-4-yl)-3,4-dihydroquinolin-1(2H)-yl)-N-methyl-1-(tetrahydro-2H-pyran-4-yl)-1,4,6,7-tetrahydro-5H-pyrazolo[4,3-c]pyridine-5-carboxamide (CPD-079/P-078)

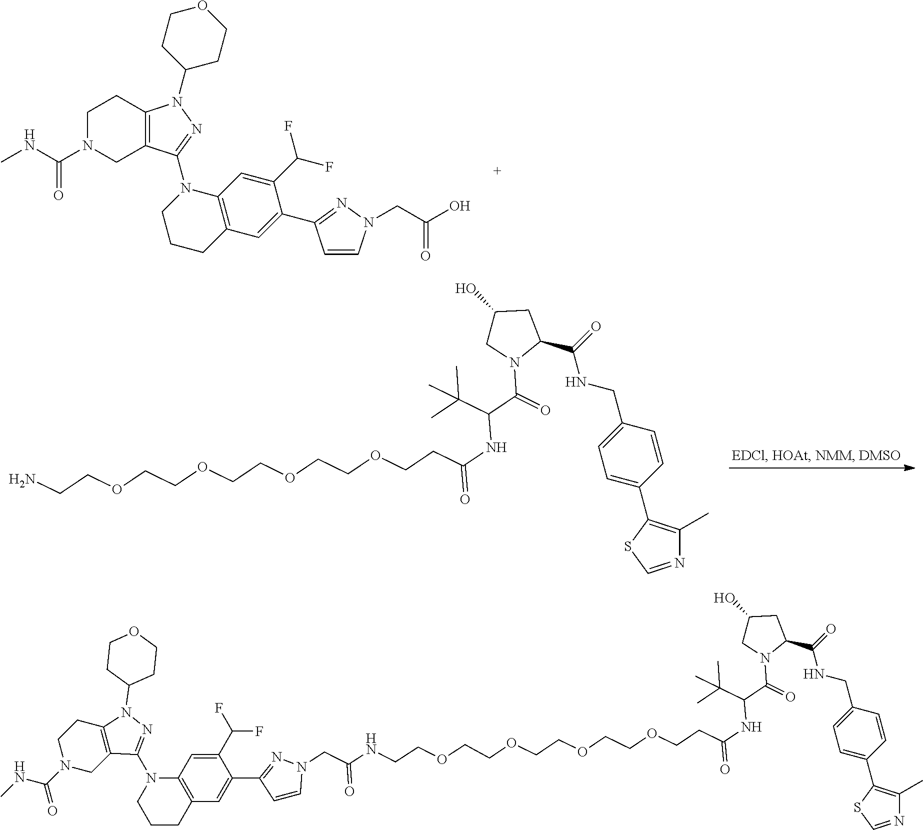

P-078 was synthesized following the standard procedure for preparing P-001 (2.8 mg, yield 26%). MS (ESI) m/z: 1230.3 [M+H]⁺.

### Example 165. 3-(7-(Difluoromethyl)-6-(1-((S)-23-((2S,4R)-4-hydroxy-2-((4-(4-methylthiazol-5-yl)benzyl)carbamoyl)pyrrolidine-1-carbonyl)-24,24-dimethyl-2,21-dioxo-6,9,12,15,18-pentaoxa-3,22-diazapentacosyl)-1H-pyrazol-4-yl)-3,4-dihydroquinolin-1(2H)-yl)-N-methyl-1-(tetrahydro-2H-pyran-4-yl)-1,4,6,7-tetrahydro-5H-pyrazolo[4,3-c]pyridine-5-carboxamide (CPD-080/P-082)

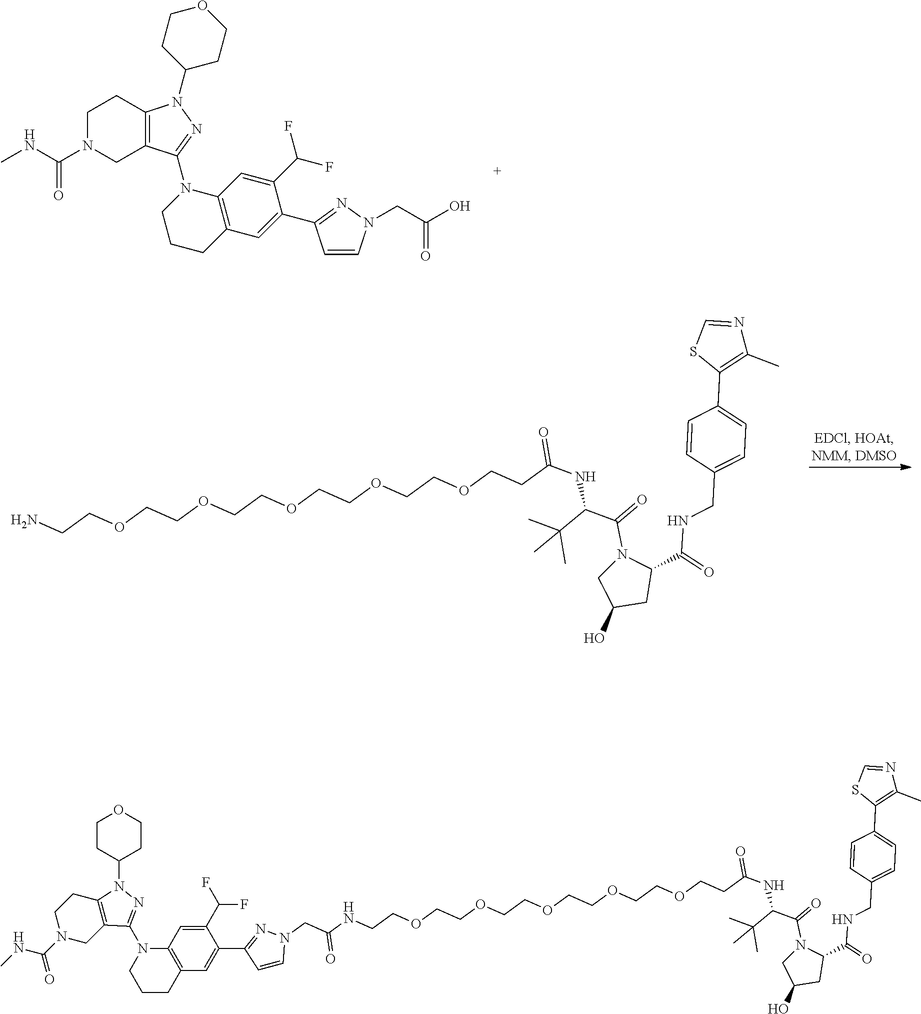

P-082 was synthesized following the standard procedure for preparing P-001 (1.9 mg, yield 17%). MS (ESI) m/z: 1274.3 [M+H]⁺.

### Example 166. 3-(7-(Difluoromethyl)-6-(1-(2-((4-(((S)-1-((2S,4R)-4-hydroxy-2-((4-(4-methylthiazol-5-yl)benzyl)carbamoyl)pyrrolidin-1-yl)-3,3-dimethyl-1-oxobutan-2-yl)amino)-4-oxobutyl)amino)-2-oxoethyl)-1H-pyrazol-4-yl)-3,4-dihydroquinolin-1(2H)-yl)-N-methyl-1-(tetrahydro-2H-pyran-4-yl)-1,4,6,7-tetrahydro-5H-pyrazolo[4,3-c]pyridine-5-carboxamide (CPD-081/P-079)

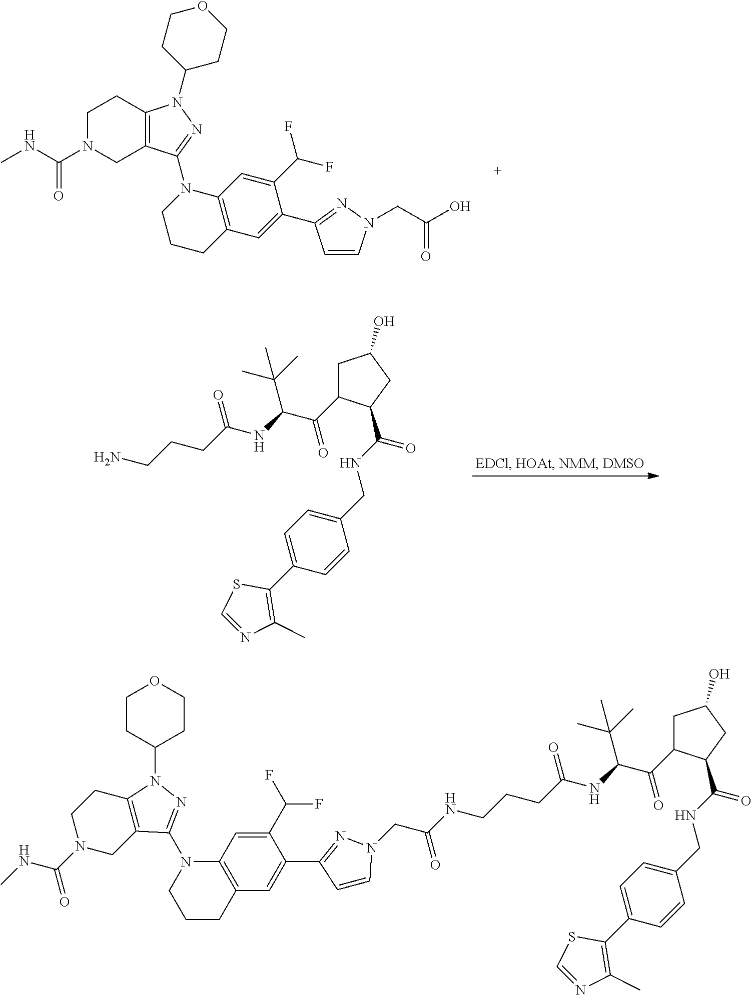 [SMILES: CNC(=O)N1CCc2c(c(N3CCCc4cc(-c5ccn(CC(=O)O)n5)c(C(F)F)cc43)nn2C2CCOCC2)C1 ; Cc1ncsc1-c1ccc(CNC(=O)[C@@H]2C[C@@H](O)CC2C(=O)[C@@H](NC(=O)CCCN)C(C)(C)C)cc1 ; CNC(=O)N1CCc2c(c(N3CCCc4cc(-c5ccn(CC(=O)NCCCC(=O)N[C@H](C(=O)C6C[C@H](O)C[C@H]6C(=O)NCc6ccc(-c7scnc7C)cc6)C(C)(C)C)n5)c(C(F)F)cc43)nn2C2CCOCC2)C1]

P-079 was synthesized following the standard procedure for preparing P-001 (2.1 mg, yield 22%). MS (ESI) m/z: 1068.1 [M+H]⁺.

### Example 167. 3-(7-(Difluoromethyl)-6-(1-(2-((7-(((S)-1-((2S,4R)-4-hydroxy-2-((4-(4-methylthiazol-5-yl)benzyl)carbamoyl)pyrrolidin-1-yl)-3,3-dimethyl-1-oxobutan-2-yl)amino)-7-oxoheptyl)amino)-2-oxoethyl)-1H-pyrazol-4-yl)-3,4-dihydroquinolin-1(2H)-yl)-N-methyl-1-(tetrahydro-2H-pyran-4-yl)-1,4,6,7-tetrahydro-5H-pyrazolo[4,3-c]pyridine-5-carboxamide (CPD-082/P-080)

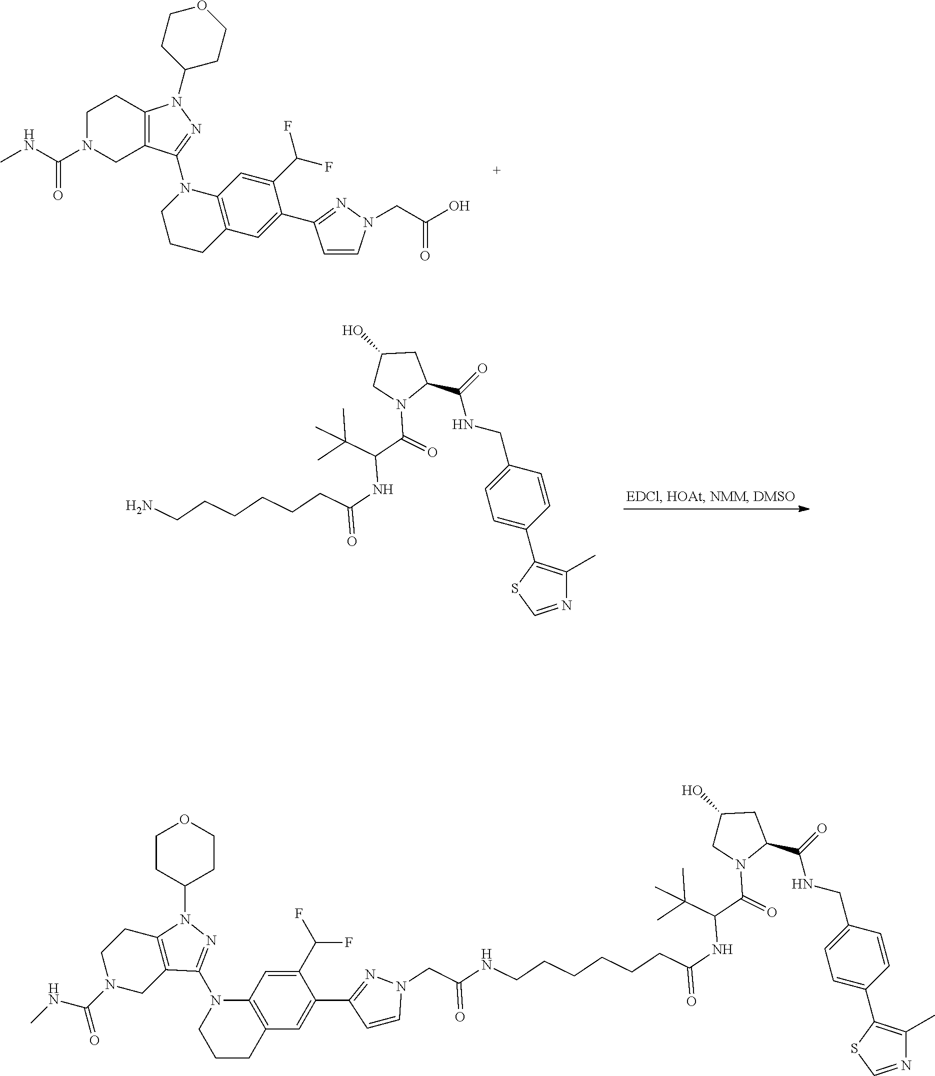

P-080 was synthesized following the standard procedure for preparing P-001 (0.84 mg, yield 9%). MS (ESI) m/z: 1110.1 [M+H]⁺.

### Example 168. 3-(7-(Difluoromethyl)-6-(1-(2-((9-(((S)-1-((2S,4R)-4-hydroxy-2-((4-(4-methylthiazol-5-yl)benzyl)carbamoyl)pyrrolidin-1-yl)-3,3-dimethyl-1-oxobutan-2-yl)amino)-9-oxononyl)amino)-2-oxoethyl)-1H-pyrazol-4-yl)-3,4-dihydroquinolin-1(2H)-yl)-N-methyl-1-(tetrahydro-2H-pyran-4-yl)-1,4,6,7-tetrahydro-5H-pyrazolo[4,3-c]pyridine-5-carboxamide (CPD-083/P-081)

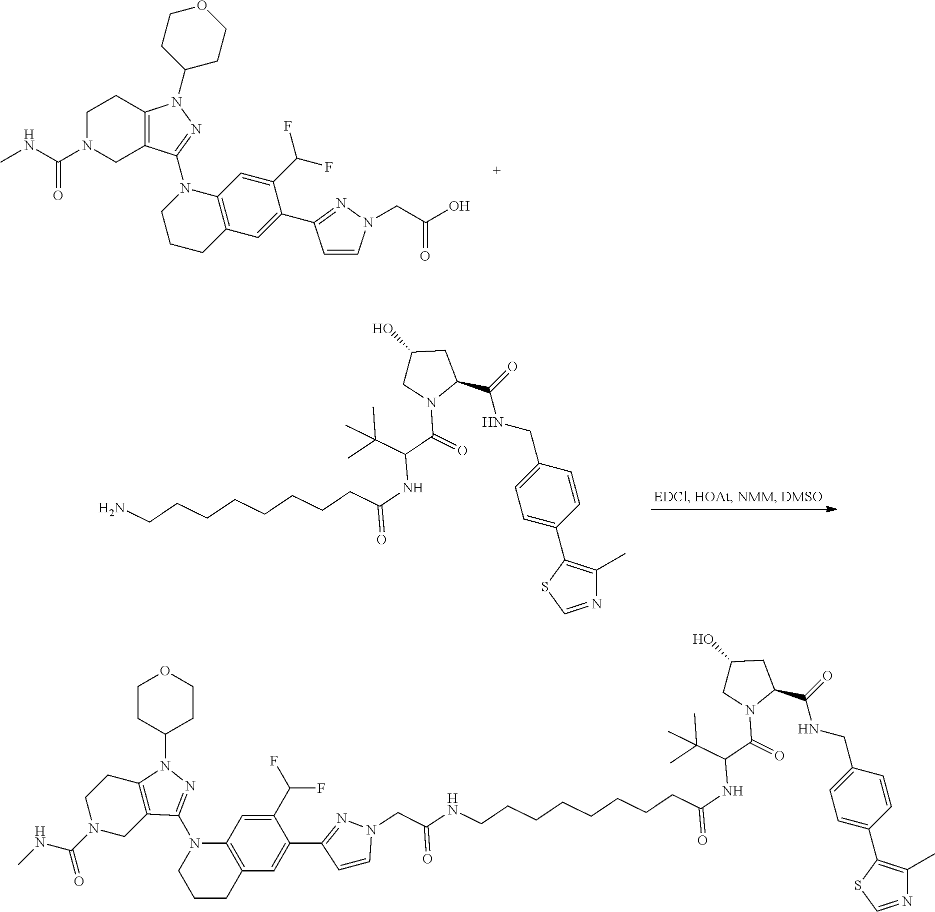

P-081 was synthesized following the standard procedure for preparing P-001 (1.6 mg, yield 16%). MS (ESI) m/z: 1138.2 [M+H]⁺.

### Example 169. 3-(7-(Difluoromethyl)-6-(1-methyl-1H-pyrazol-4-yl)-3,4-dihydroquinolin-1(2H)-yl)-1-(1-(6-((2-(2,6-dioxopiperidin-3-yl)-1,3-dioxoisoindolin-4-yl)oxy)hexanoyl)piperidin-4-yl)-N-methyl-1,4,6,7-tetrahydro-5H-pyrazolo[4,3-c]pyridine-5-carboxamide (P-084)

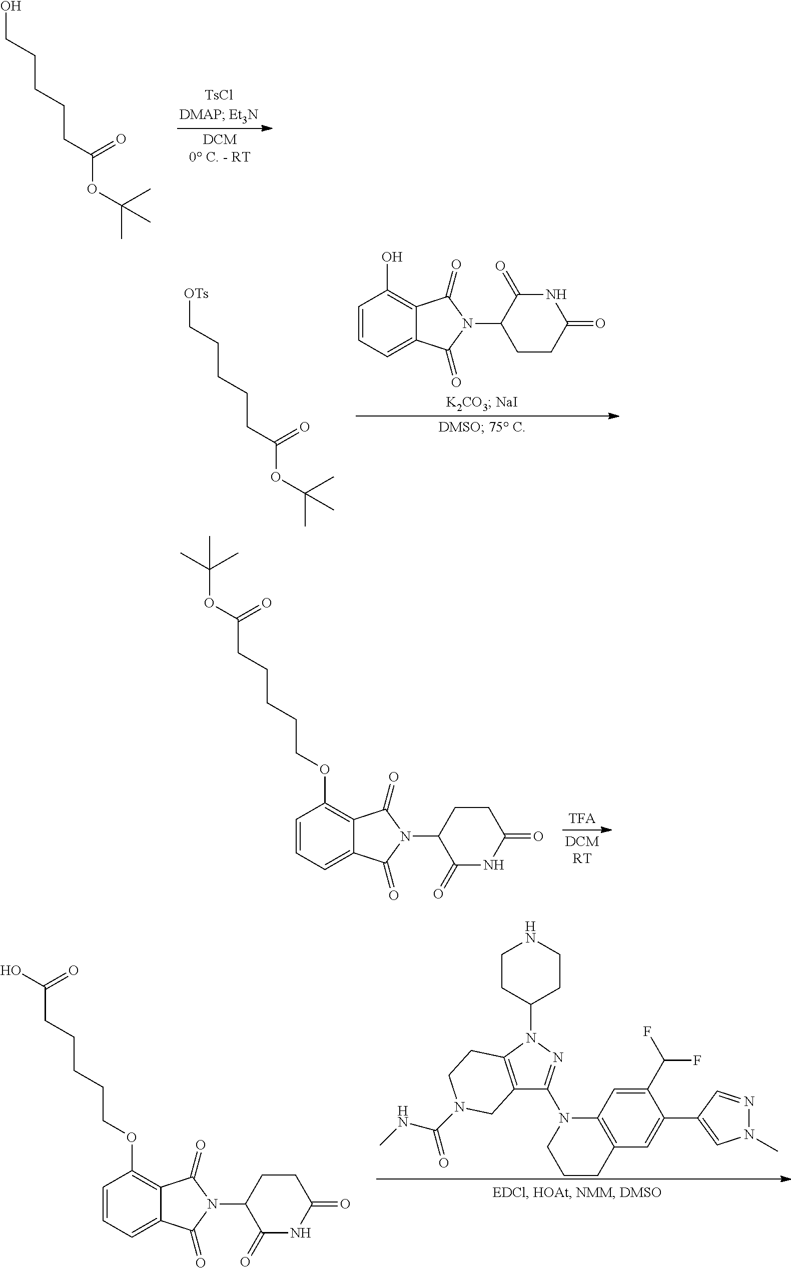

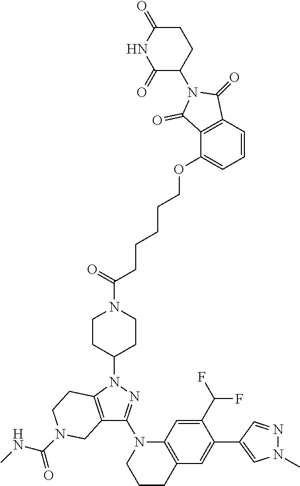

### Step 1: Synthesis of tert-butyl 6-(tosyloxy)hexanoate

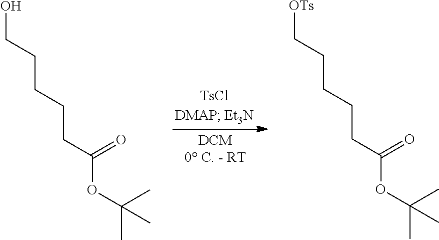 [SMILES: CC(C)(C)OC(=O)CCCCCO ; Cc1ccc(S(=O)(=O)OCCCCCC(=O)OC(C)(C)C)cc1]

To a solution of tert-butyl 6-hydroxyhexanoate (500 mg, 2.66 mmol), DMAP (16 mg, 0.13 mmol) and TEA (806 mg, 7.97 mmol) in DCM (45 mL) was added TsCl (608 mg, 3.19 mmol) at 0° C. After the reaction was stirred at room temperature for 2 h, the reaction mixture was washed with water, dried over Na₂SO₄, filtered and concentrated. The residue was purified by silica gel column chromatography to give the desired product (700 mg, yield 77%) as a colorless oil.

### Step 2: Synthesis of tert-butyl 6-((2-(2,6-dioxopiperidin-3-yl)-1,3-dioxoisoindolin-4-yl)oxy)hexanoate

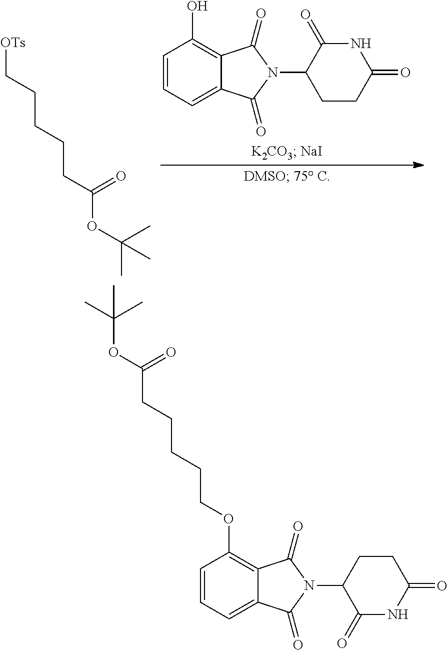

To a solution of tert-butyl 6-(tosyloxy)hexanoate (600 mg, 1.75 mmol) and 2-(2,6-dioxopiperidin-3-yl)-4-hydroxyisoindoline-1,3-dione (480 mg, 1.75 mmol) in DMSO (6 mL) were added K₂CO₃ (484 mg, 3.5 mmol) and Nat (394 mg, 2.63 mmol). The resulting mixture was stirred at 80° C. for 3 h, before the reaction mixture was filtered. The filtrate was directly purified by reverse phase chromatography to give the desired product (440 mg, yield 57%) as a white solid. (ESI) m/z: 388.4 [M−^{t}Bu+H]⁺.

### Step 3: Synthesis of 6-((2-(2,6-dioxopiperidin-3-yl)-1,3-dioxoisoindolin-4-yl)oxy)hexanoic acid

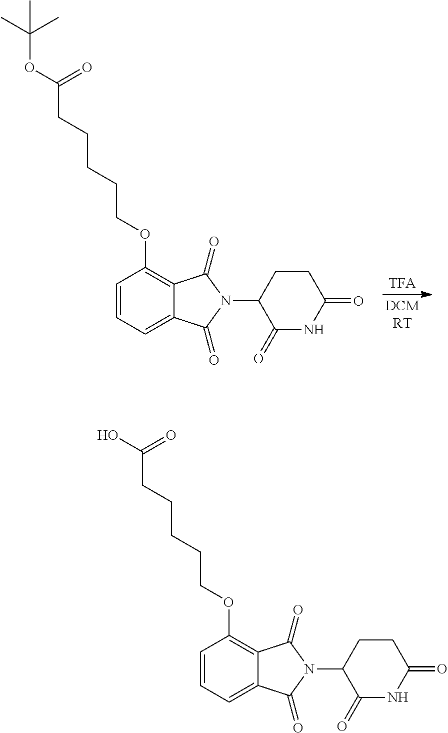 [SMILES: CC(C)(C)OC(=O)CCCCCOc1cccc2c1C(=O)N(C1CCC(=O)NC1=O)C2=O ; O=C(O)CCCCCOc1cccc2c1C(=O)N(C1CCC(=O)NC1=O)C2=O]

A mixture of tert-butyl 6-((2-(2,6-dioxopiperidin-3-yl)-1,3-dioxoisoindolin-4-yl)oxy)hexanoate (440 mg, 0.99 mmol) in DCM (8 mL) and TFA (1 mL) was stirred at room temperature for 2 h. The reaction mixture was concentrated to give the crude product (496 mg, yield 99%) as a white solid which was used directly in the next step.

### Step 4: Synthesis of 3-(7-(difluoromethyl)-6-(1-methyl-1H-pyrazol-4-yl)-3,4-dihydroquinolin-1(2H)-yl)-1-(1-(6-((2-(2,6-dioxopiperidin-3-yl)-1,3-dioxoisoindolin-4-yl)oxy)hexanoyl)piperidin-4-yl)-N-methyl-1,4,6,7-tetrahydro-5H-pyrazolo[4,3-c]pyridine-5-carboxamide (P-084)

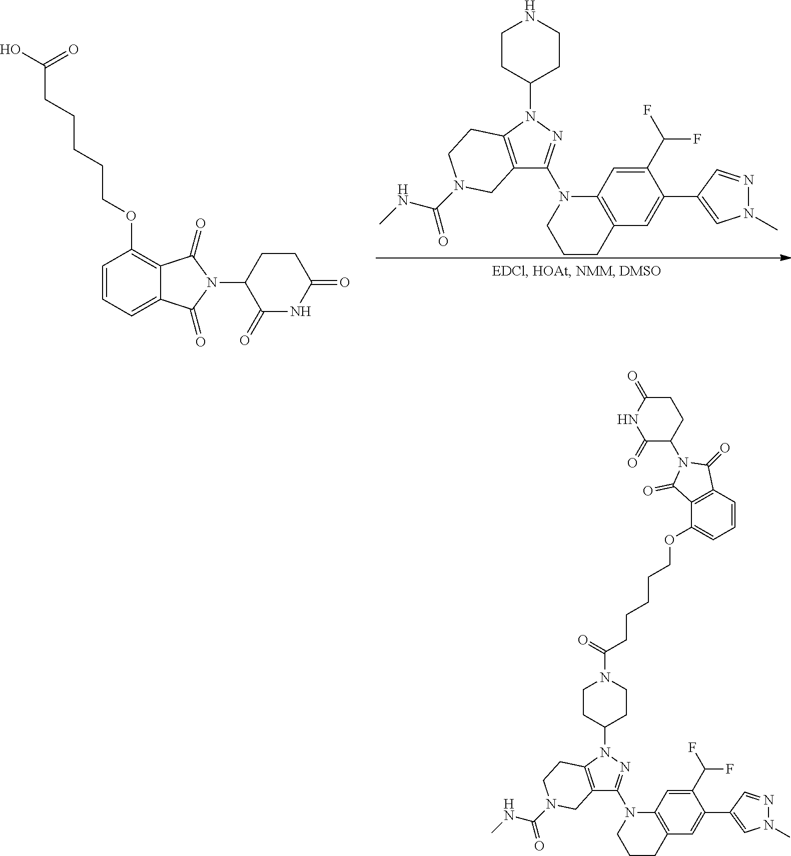

P-084 was synthesized following the standard procedure for preparing P-001 (6.8 mg, yield 51%). MS (ESI) m/z: 895.7 [M+H]⁺.

### Example 170. 3-(7-(Difluoromethyl)-6-(1-methyl-1H-pyrazol-4-yl)-3,4-dihydroquinolin-1(2H)-yl)-1-(1-(7-(2-(2,6-dioxopiperidin-3-yl)-1,3-dioxoisoindolin-4-yl)heptanoyl)piperidin-4-yl)-N-methyl-1,4,6,7-tetrahydro-5H-pyrazolo[4,3-c]pyridine-5-carboxamide (P-085)

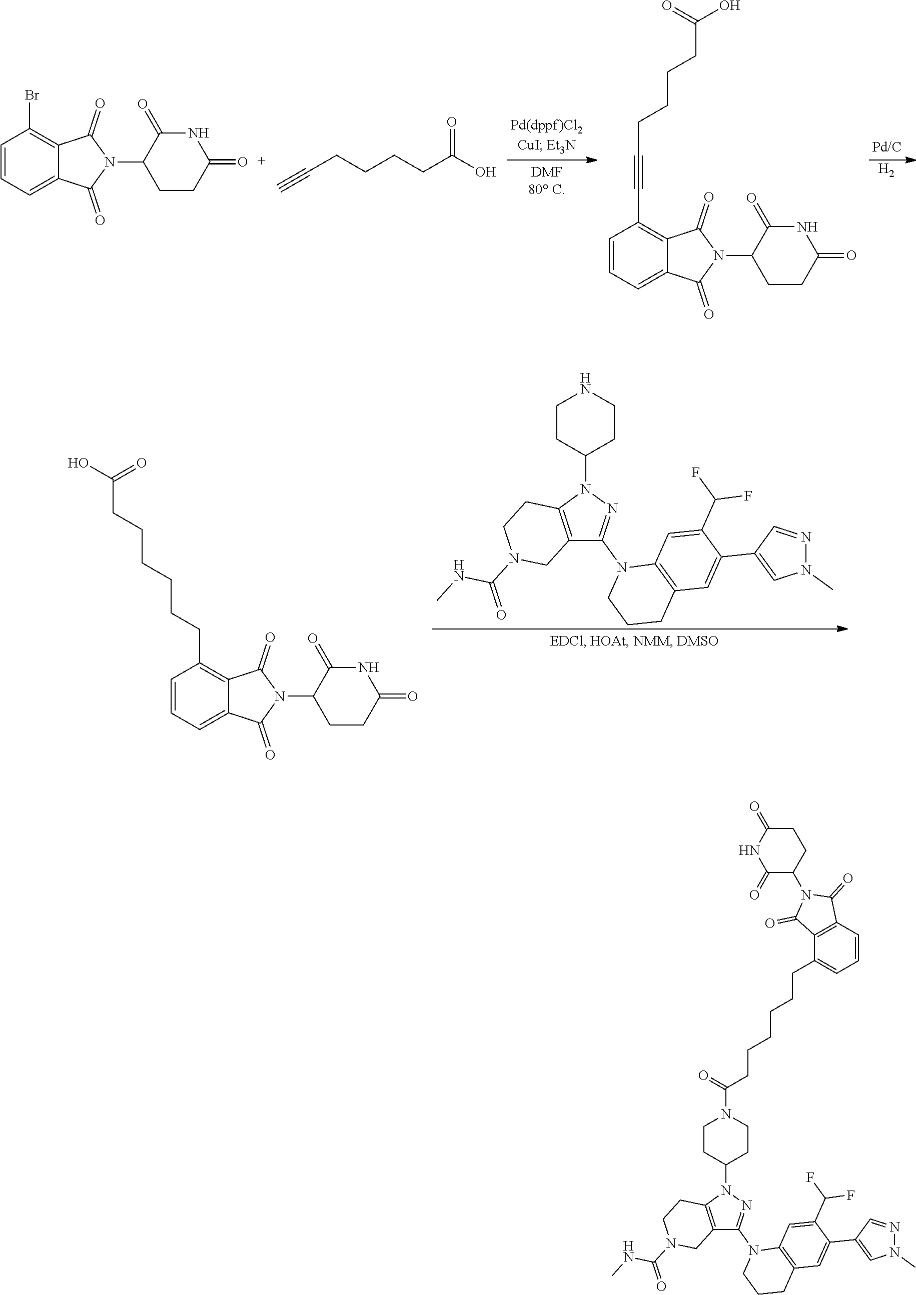

### Step 1: Synthesis of 7-(2-(2,6-dioxopiperidin-3-yl)-1,3-dioxoisoindolin-4-yl)hept-6-ynoic acid

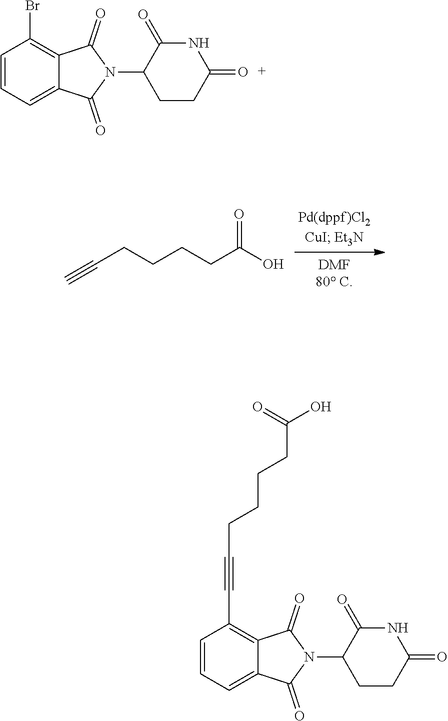

To a solution of 4-bromo-2-(2,6-dioxopiperidin-3-yl)isoindoline-1,3-dione (250 mg, 0.74 mmol) in DMF (5 mL) were added Pd(dppf)Cl₂ (27.1 mg, 0.037 mmol), CuI (14.1 mg, 0.074 mmol), TEA (75 mg, 0.074 mmol) and hept-6-ynoic acid (103 mg, 0.082 mmol). The resulting mixture was stirred at 85° C. for 2.5 h under N₂. The reaction mixture was purified by reverse phase chromatography to give the desired product (200 mg, yield 71%) as a white solid. MS (ESI) m/z: 383.3 [M+H]⁺.

### Step 2: Synthesis of 7-(2-(2,6-dioxopiperidin-3-yl)-1,3-dioxoisoindolin-4-yl)heptanoic acid

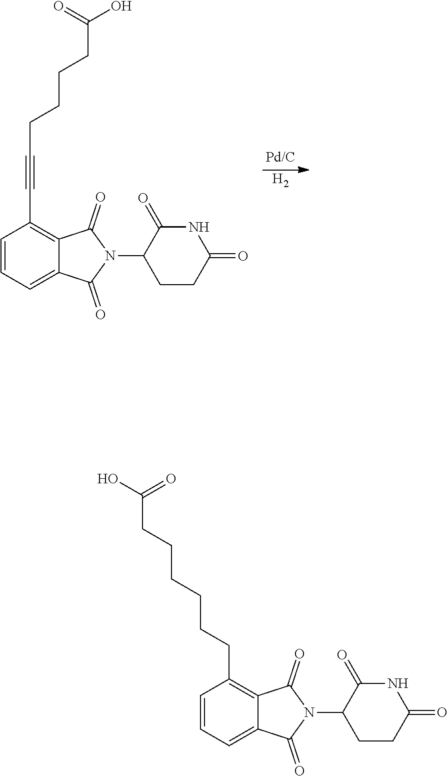 [SMILES: O=C(O)CCCCC#Cc1cccc2c1C(=O)N(C1CCC(=O)NC1=O)C2=O ; "H2" ; O=C(O)CCCCCCc1cccc2c1C(=O)N(C1CCC(=O)NC1=O)C2=O]

A mixture of 7-(2-(2,6-dioxopiperidin-3-yl)-1,3-dioxoisoindolin-4-yl)hept-6-ynoic acid (200 mg, 0.52 mmol) and Pd/C (30 mg) in THF (10 mL) was stirred at room temperature for 2 h under H2. After the reaction mixture was filtered through Celite, the filtrate was concentrated to give the desired product (202 mg, yield 99%) as a white solid. MS (ESI) m/z: 387.5 [M+H]⁺.

### Step 3: Synthesis of 3-(7-(difluoromethyl)-6-(1-methyl-1H-pyrazol-4-yl)-3,4-dihydroquinolin-1(2H)-yl)-1-(1-(7-(2-(2,6-dioxopiperidin-3-yl)-1,3-dioxoisoindolin-4-yl)heptanoyl)piperidin-4-yl)-N-methyl-1,4,6,7-tetrahydro-5H-pyrazolo[4,3-c]pyridine-5-carboxamide (P-085)

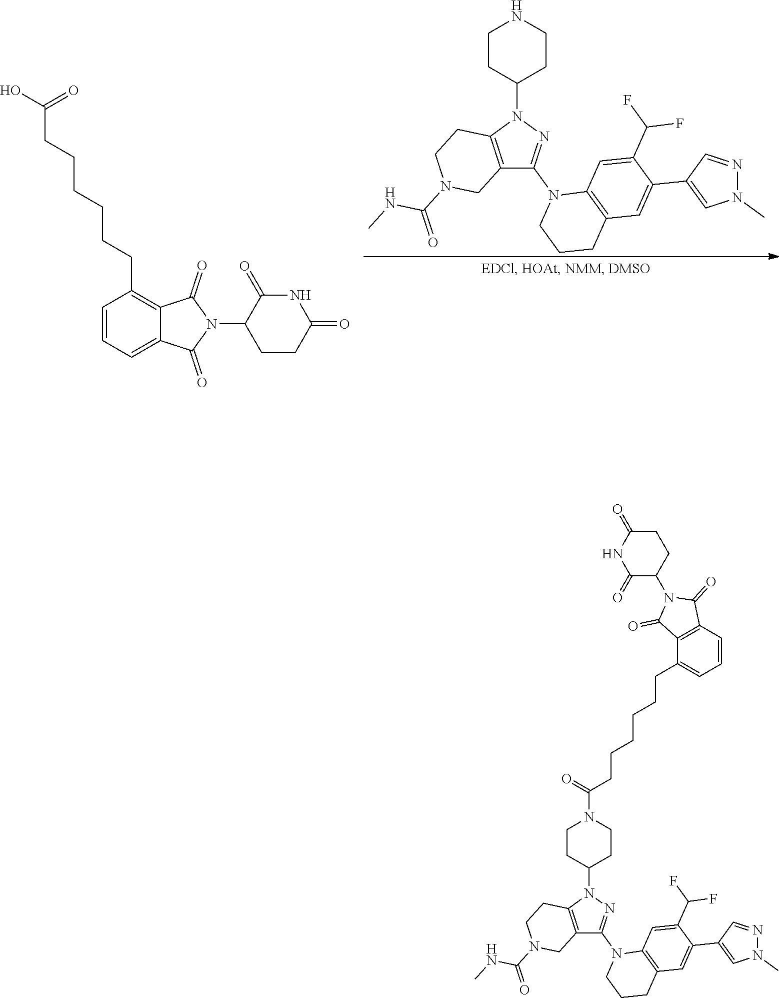

P-085 was synthesized following the standard procedure for preparing P-001 (6 mg, yield 45%). MS (ESI) m/z: 893.8 [M+H]⁺.

### Example 171. 3-(7-(Difluoromethyl)-6-(1-methyl-1H-pyrazol-4-yl)-3,4-dihydroquinolin-1(2H)-yl)-1-(1-(7-(2-(2,6-dioxopiperidin-3-yl)-1-oxoisoindolin-4-yl)heptanoyl)piperidin-4-yl)-N-methyl-1,4,6,7-tetrahydro-5H-pyrazolo[4,3-c]pyridine-5-carboxamide (P-086)

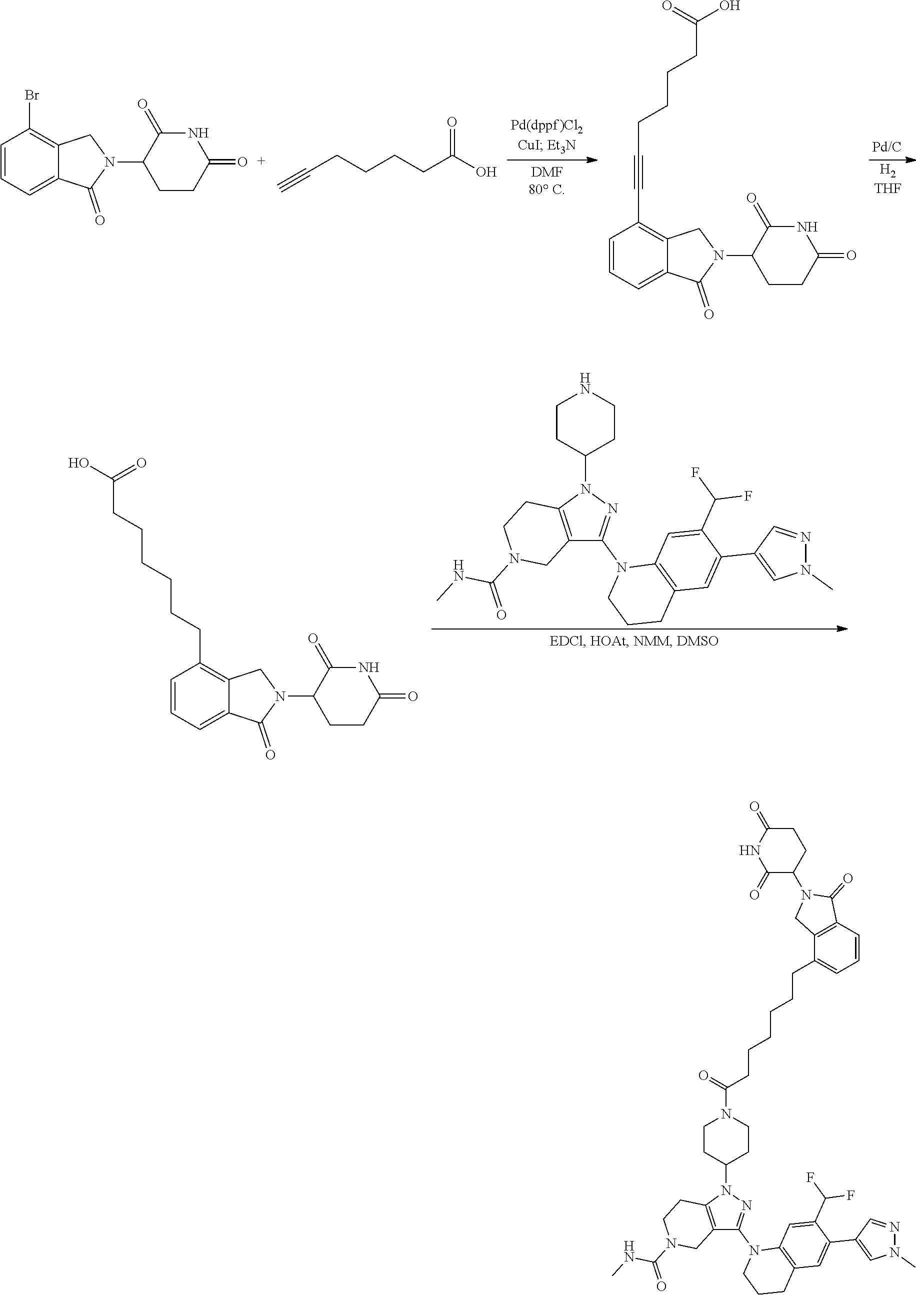

P-086 was synthesized following the similar procedure for preparing P-085 (6 mg, yield 46%). MS (ESI) m/z: 879.8 [M+H]⁺.

### Example 172. 3-(7-(Difluoromethyl)-6-(1-methyl-1H-pyrazol-4-yl)-3,4-dihydroquinolin-1(2H)-yl)-1-(1-(7-(2-(2,6-dioxopiperidin-3-yl)-3-oxoisoindolin-4-yl)heptanoyl)piperidin-4-yl)-N-methyl-1,4,6,7-tetrahydro-5H-pyrazolo[4,3-c]pyridine-5-carboxamide (P-087)

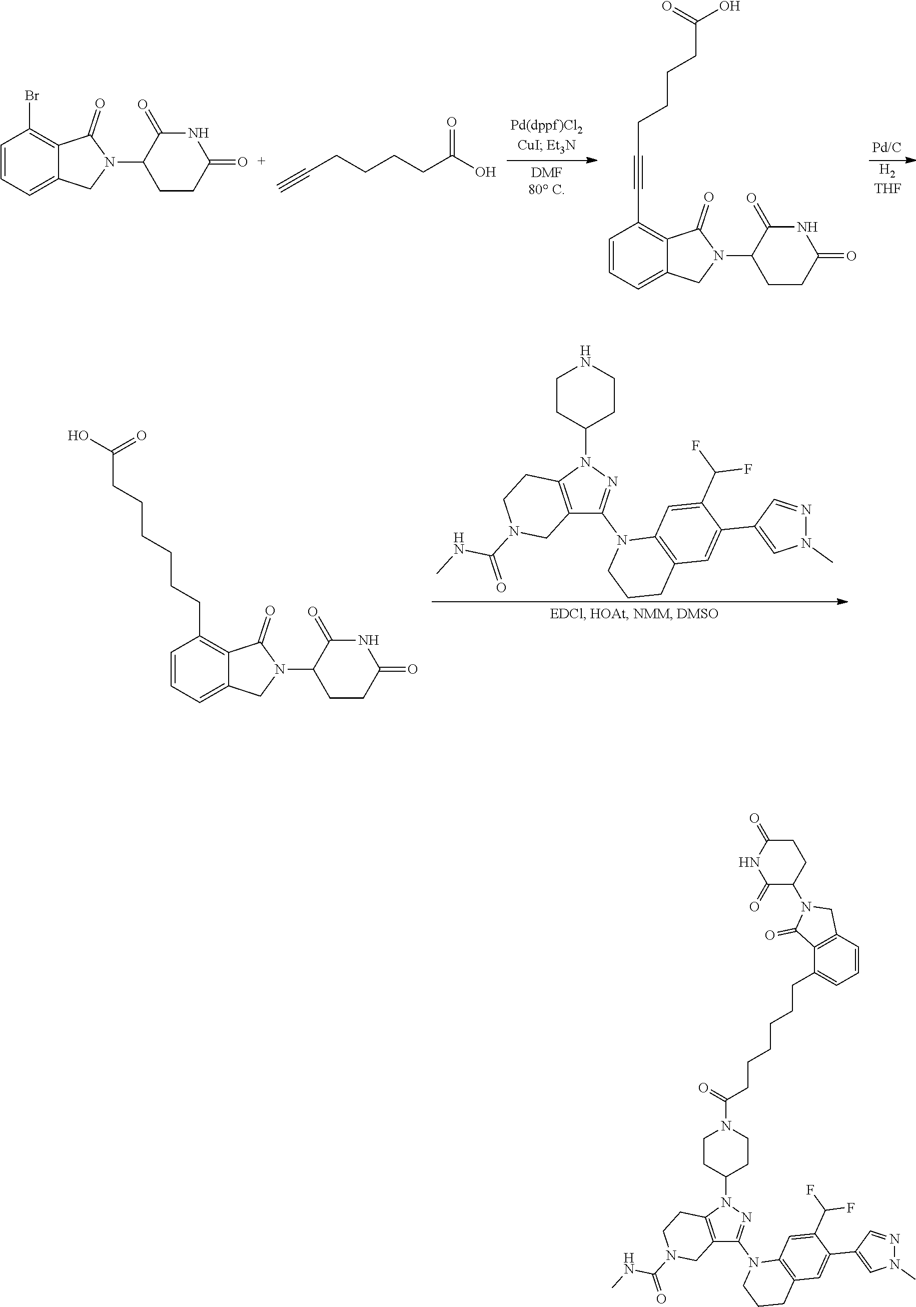

P-087 was synthesized following the similar procedure for preparing P-085 (6.8 mg, yield 52%). MS (ESI) m/z: 879.8 [M+H]⁺.

### Example 173. 3-(7-(Difluoromethyl)-6-(1-methyl-1H-pyrazol-4-yl)-3,4-dihydroquinolin-1(2H)-yl)-1-(1-(6-((2-(2,6-dioxopiperidin-3-yl)-1-oxoisoindolin-4-yl)oxy)hexanoyl)piperidin-4-yl)-N-methyl-1,4,6,7-tetrahydro-5H-pyrazolo[4,3-c]pyridine-5-carboxamide (P-088)

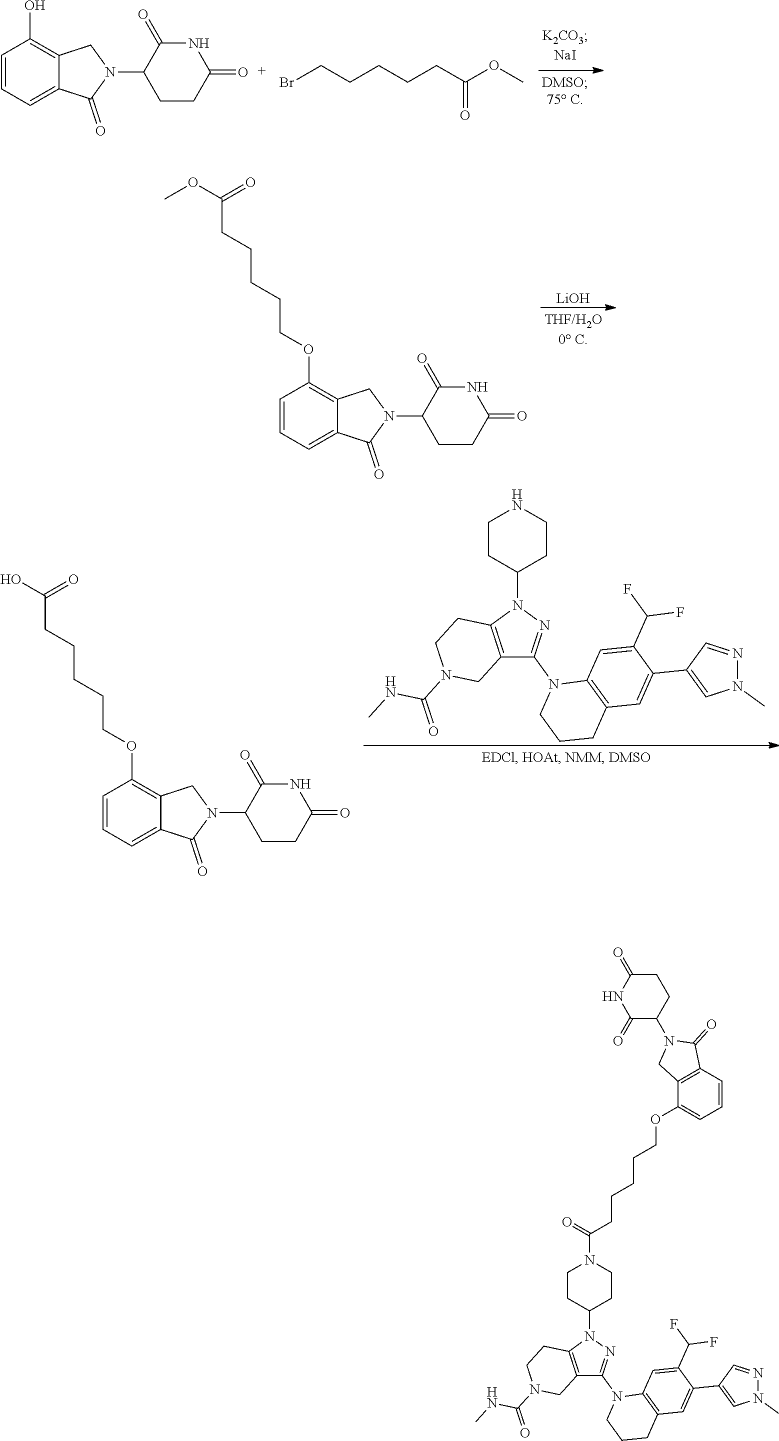

P-088 was synthesized following the similar procedure for preparing P-084 (3 mg, yield 26%). MS (ESI) m/z: 881.9 [M+H]⁺.

### Example 174. 3-(7-(Difluoromethyl)-6-(1-methyl-1H-pyrazol-4-yl)-3,4-dihydroquinolin-1(2H)-yl)-1-(1-(6-((2-(2,6-dioxopiperidin-3-yl)-3-oxoisoindolin-4-yl)oxy)hexanoyl)piperidin-4-yl)-N-methyl-1,4,6,7-tetrahydro-5H-pyrazolo[4,3-c]pyridine-5-carboxamide (P-089)

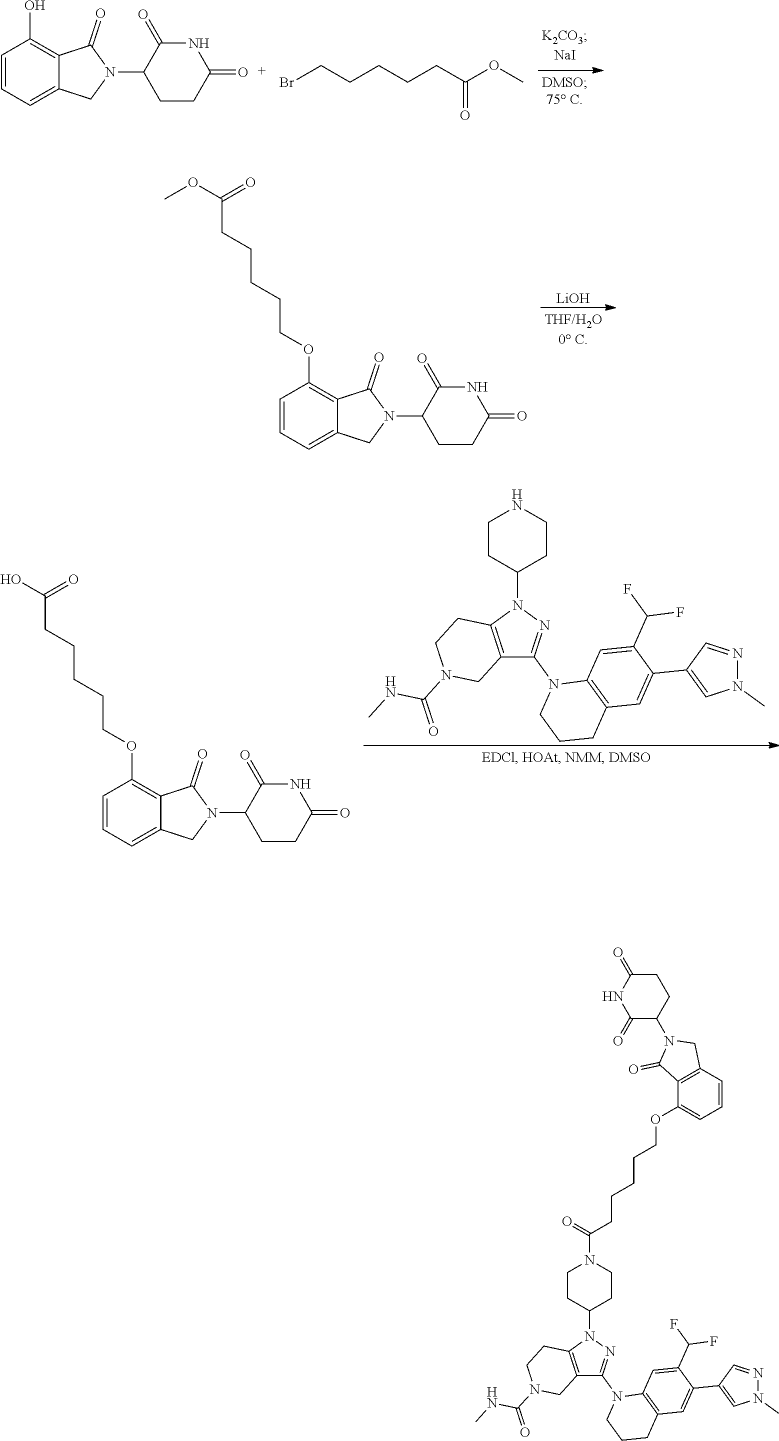

P-089 was synthesized following the similar procedure for preparing P-084 (5.1 mg, yield 57%). MS (ESI) m/z: 881.8 [M+H]⁺.

### Example 175. 3-(7-(Difluoromethyl)-6-(1-methyl-1H-pyrazol-4-yl)-3,4-dihydroquinolin-1(2H)-yl)-1-(1-(6-((2-(2,6-dioxopiperidin-3-yl)-1,3-dioxoisoindolin-4-yl)amino)hexyl)piperidin-4-yl)-N-methyl-1,4,6,7-tetrahydro-5H-pyrazolo[4,3-c]pyridine-5-carboxamide (P-090)

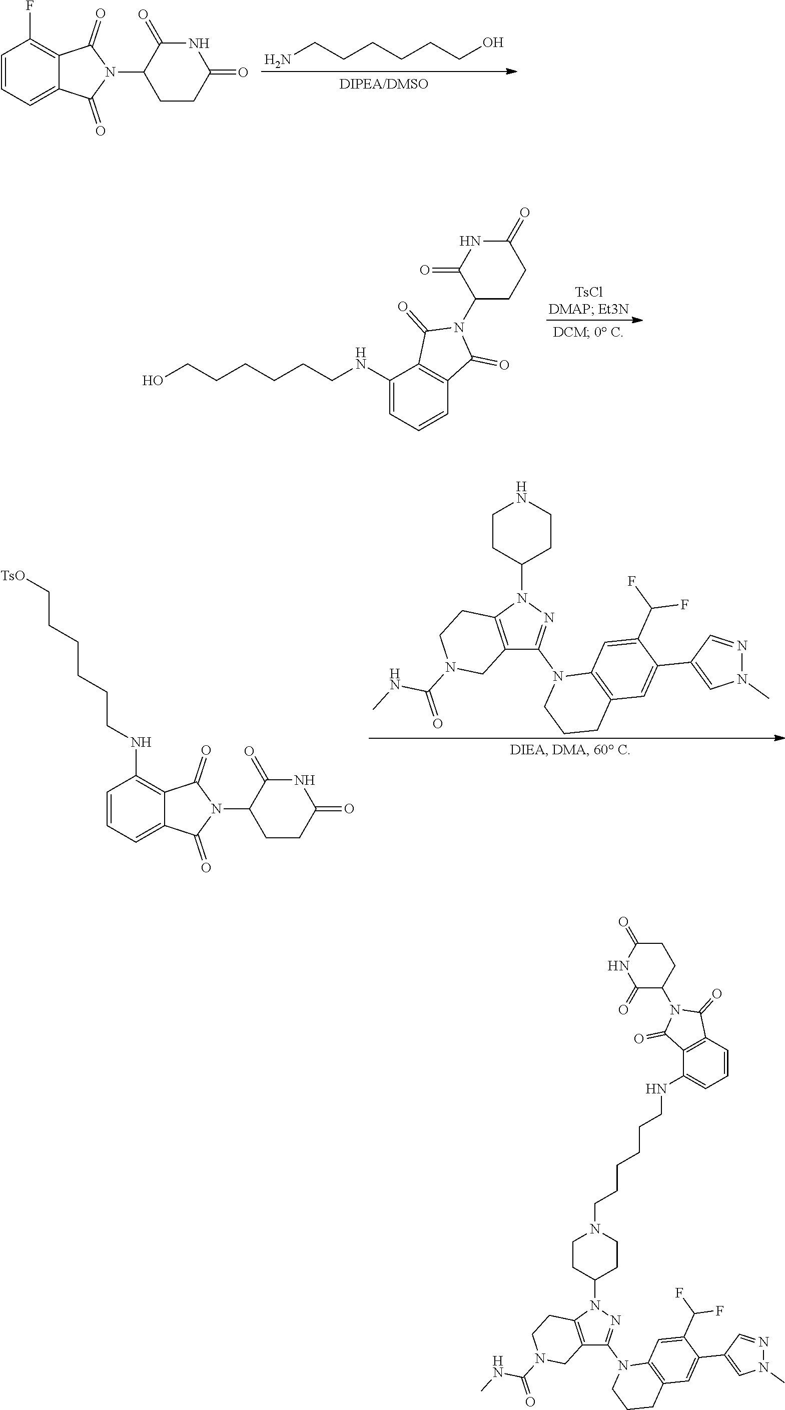

### Step 1: Synthesis of 2-(2,6-dioxopiperidin-3-yl)-4-((6-hydroxyhexyl)amino)isoindoline-1,3-dione

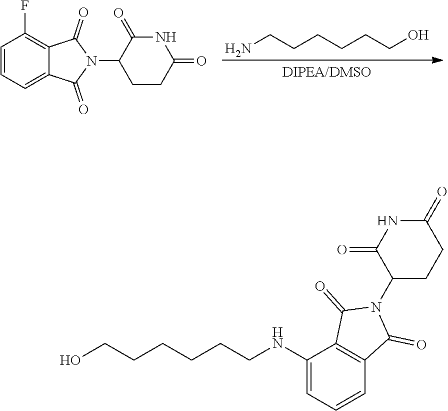 [SMILES: O=C1CCC(N2C(=O)c3cccc(F)c3C2=O)C(=O)N1 ; NCCCCCCO ; O=C1CCC(N2C(=O)c3cccc(NCCCCCCO)c3C2=O)C(=O)N1]

To a solution of 2-(2,6-dioxopiperidin-3-yl)-4-fluoroisoindoline-1,3-dione (500 mg, 1.81 mmol) and 6-aminohexan-1-ol (212 mg, 1.81 mmol) in DMSO (10 mL) was added DIEA (702 mg, 5.43 mmol). The resulting mixture was stirred at 120° C. for 2 h. After cooling to room temperature, the reaction mixture was purified by prep-HPLC to give the desired product (300 mg, yield 44%) as a yellow solid. MS (ESI) m/z: 374.5 [M+H]⁺.

### Step 2: Synthesis of 6-((2-(2,6-dioxopiperidin-3-yl)-1,3-dioxoisoindolin-4-yl)amino)hexyl 4-methylbenzenesulfonate

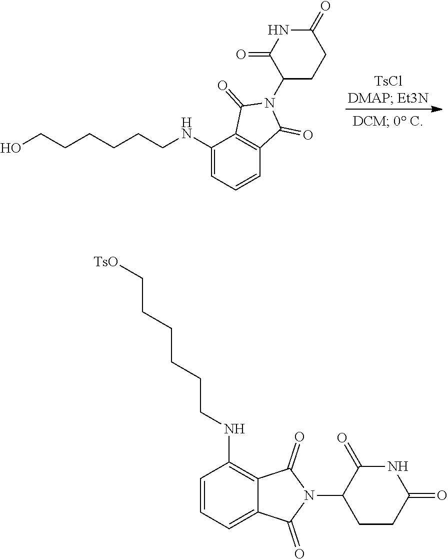 [SMILES: O=C1CCC(N2C(=O)c3cccc(NCCCCCCO)c3C2=O)C(=O)N1 ; "TsClDMAP; Et3N" ; Cc1ccc(S(=O)(=O)OCCCCCCNc2cccc3c2C(=O)N(C2CCC(=O)NC2=O)C3=O)cc1]

To a solution of 2-(2,6-dioxopiperidin-3-yl)-4-((6-hydroxyhexyl)amino)isoindoline-1,3-dione (250 mg, 0.67 mmol) in DCM (15 mL) were added DMAP (4 mg, 0.03 mmol), TEA (202 mg, 2.0 mmol) and TsCl (191 mg, 1.0 mmol) at 0° C. The resulting mixture was stirred at room temperature for 2 h, before the reaction mixture was concentrated. The residue was purified by silica gel column chromatography to give the desired product (270 mg, yield 77%) as a white solid. MS (ESI) m/z: 528.5 [M+H]⁺.

### Step 3: Synthesis of 3-(7-(difluoromethyl)-6-(1-methyl-1H-pyrazol-4-yl)-3,4-dihydroquinolin-1(2H)-yl)-1-(1-(6-((2-(2,6-dioxopiperidin-3-yl)-1,3-dioxoisoindolin-4-yl)amino)hexyl)piperidin-4-yl)-N-methyl-1,4,6,7-tetrahydro-5H-pyrazolo[4,3-c]pyridine-5-carboxamide (P-090)

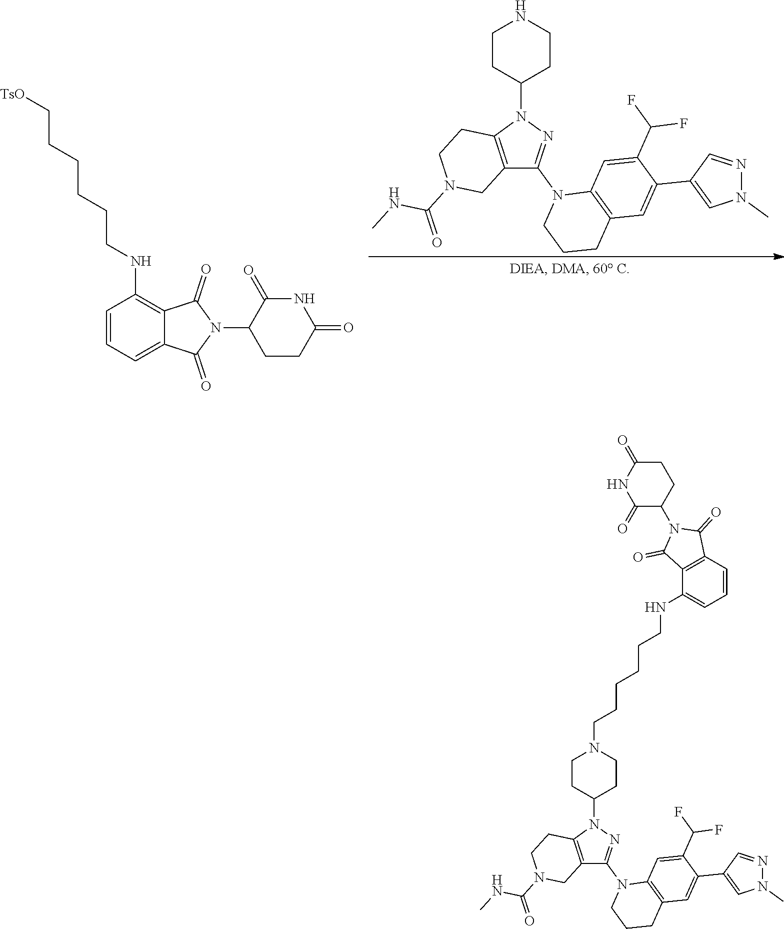

A mixture of 6-((2-(2,6-dioxopiperidin-3-yl)-1,3-dioxoisoindolin-4-yl)amino)hexyl 4-methylbenzenesulfonate (4 mg, 0.00758 mmol), 3-(7-(difluoromethyl)-6-(1-methyl-1H-pyrazol-4-yl)-3,4-dihydroquinolin-1(2H)-yl)-N-methyl-1-(piperidin-4-yl)-1,4,6,7-tetrahydro-5H-pyrazolo[4,3-c]pyridine-5-carboxamide (3.98 mg, 0.00758 mmol) and DIEA (4.85 mg, 0.038 mmol) in DMA (0.5 mL) was stirred at 60° C. for 4 h. The reaction mixture was purified by reverse phase chromatography to give desired product (2 mg, yield 30%) as a white solid. MS (ESI) m/z: 880.9 [M+H]⁺.

### Example 176. 3-(7-(Difluoromethyl)-6-(1-methyl-1H-pyrazol-4-yl)-3,4-dihydroquinolin-1(2H)-yl)-1-(1-(6-((2-(2,6-dioxopiperidin-3-yl)-1,3-dioxoisoindolin-4-yl)oxy)hexyl)piperidin-4-yl)-N-methyl-1,4,6,7-tetrahydro-5H-pyrazolo[4,3-c]pyridine-5-carboxamide (P-091)

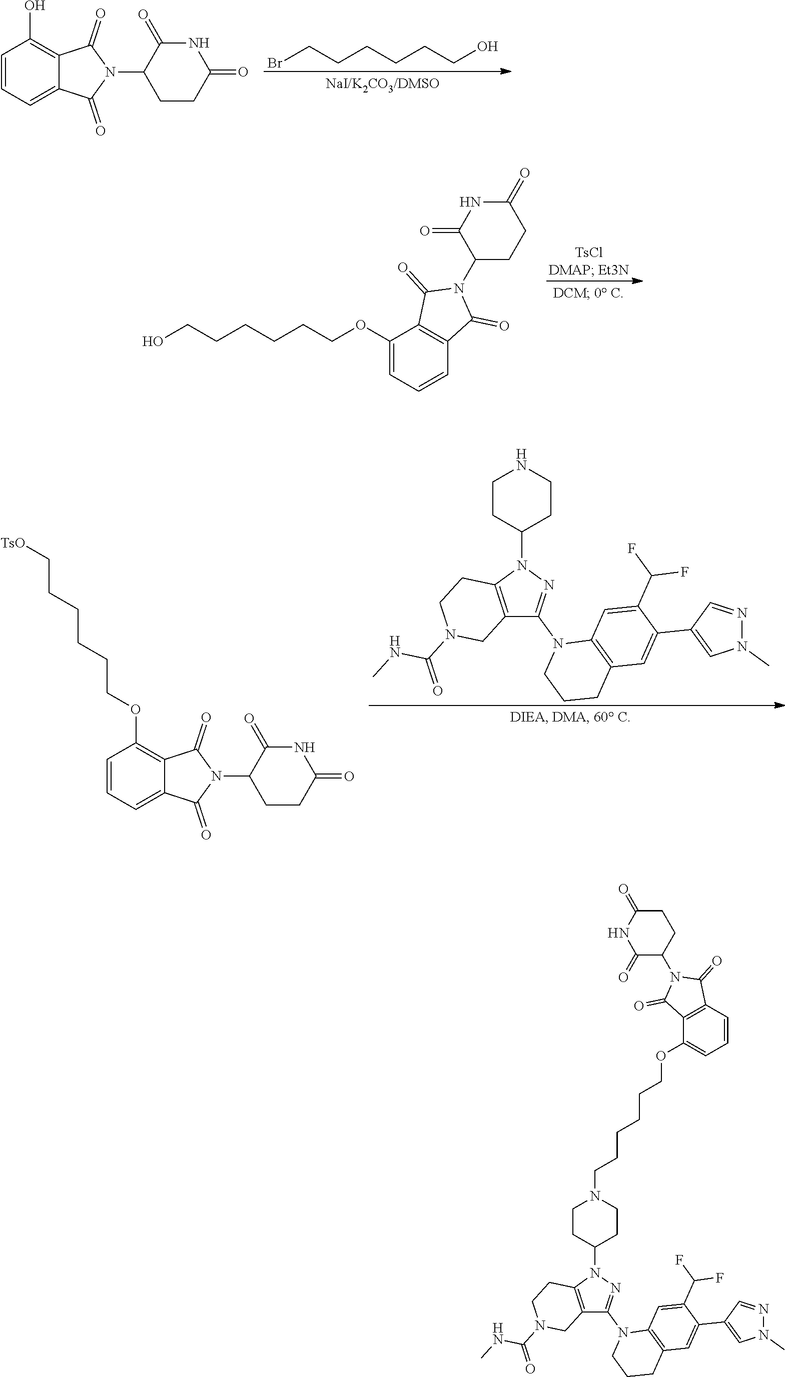

P-091 was synthesized following the similar procedure for preparing P-090 (3 mg, yield 45%). MS (ESI) m/z: 881.8 [M+H]⁺.

### Example 177. 3-(7-(Difluoromethyl)-6-(1-methyl-1H-pyrazol-4-yl)-3,4-dihydroquinolin-1(2H)-yl)-1-(1-(7-(2-(2,6-dioxopiperidin-3-yl)-1,3-dioxoisoindolin-4-yl)hept-6-ynoyl)piperidin-4-yl)-N-methyl-1,4,6,7-tetrahydro-5H-pyrazolo[4,3-c]pyridine-5-carboxamide (P-092)

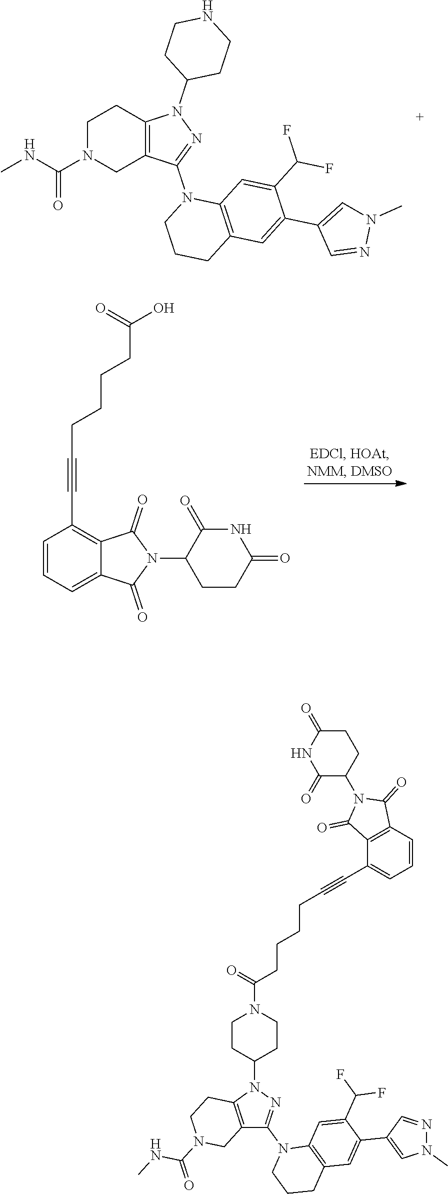

P-092 was synthesized following the standard procedure for preparing P-001 (5.3 mg, yield 62%). MS (ESI) m/z: 889.8 [M+H]⁺.

### Example 178. 3-(7-(Difluoromethyl)-6-(1-methyl-1H-pyrazol-4-yl)-3,4-dihydroquinolin-1(2H)-yl)-1-(1-(7-(2-(2,6-dioxopiperidin-3-yl)-1-oxoisoindolin-4-yl)hept-6-ynoyl)piperidin-4-yl)-N-methyl-1,4,6,7-tetrahydro-5H-pyrazolo[4,3-c]pyridine-5-carboxamide (P-093)

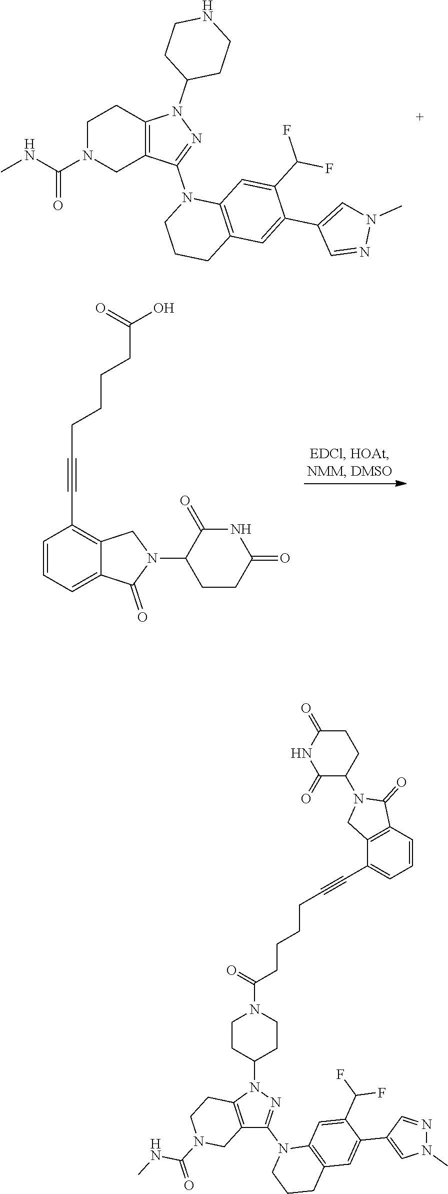

P-093 was synthesized following the standard procedure for preparing P-001 (5.0 mg, yield 55%). MS (ESI) m/z: 875.8 [M+H]⁺.

### Example 179. 3-(7-(Difluoromethyl)-6-(1-methyl-1H-pyrazol-4-yl)-3,4-dihydroquinolin-1(2H)-yl)-1-(1-(7-(2-(2,6-dioxopiperidin-3-yl)-3-oxoisoindolin-4-yl)hept-6-ynoyl)piperidin-4-yl)-N-methyl-1,4,6,7-tetrahydro-5H-pyrazolo[4,3-c]pyridine-5-carboxamide (P-094)

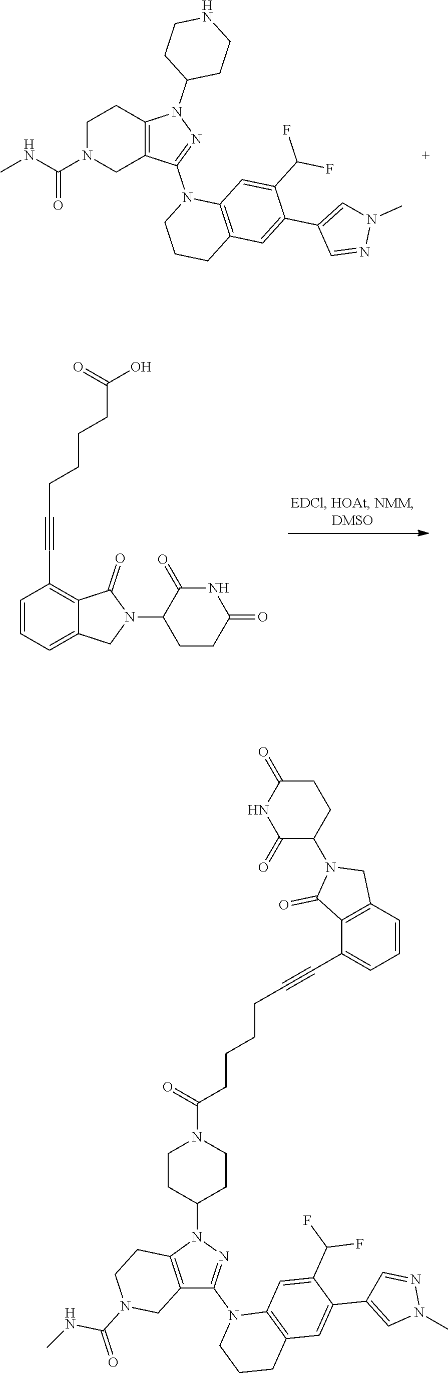

P-094 was synthesized following the standard procedure for preparing P-001 (4.6 mg, yield 51%). MS (ESI) m/z: 875.8 [M+H]⁺.

### Example 180. 3-(7-(Difluoromethyl)-6-(1-methyl-1H-pyrazol-4-yl)-3,4-dihydroquinolin-1(2H)-yl)-1-(1-(6-((2-(2,6-dioxopiperidin-3-yl)-1-oxoisoindolin-4-yl)amino)hexanoyl)piperidin-4-yl)-N-methyl-1,4,6,7-tetrahydro-5H-pyrazolo[4,3-c]pyridine-5-carboxamide (P-095)

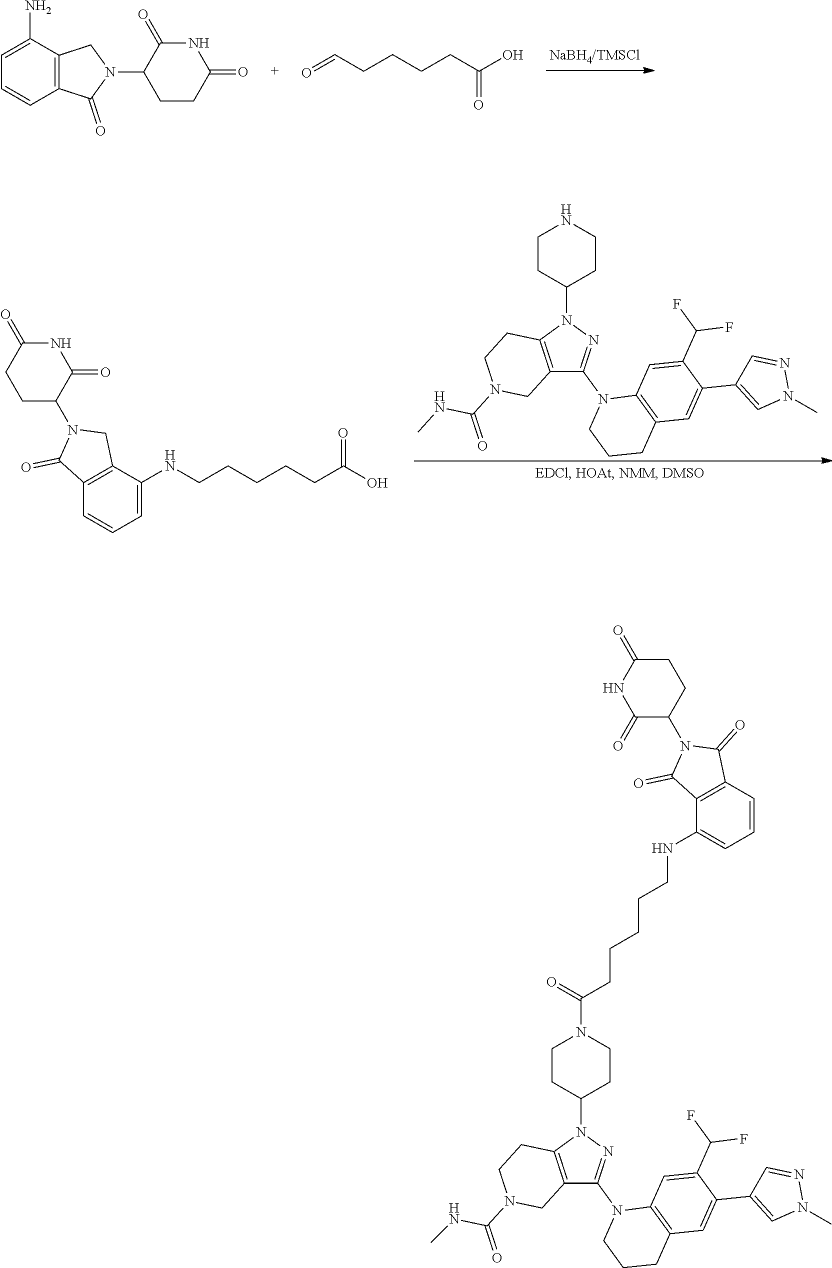

### Step 1: Synthesis of 6-((2-(2,6-dioxopiperidin-3-yl)-1-oxoisoindolin-4-yl)amino)hexanoic acid

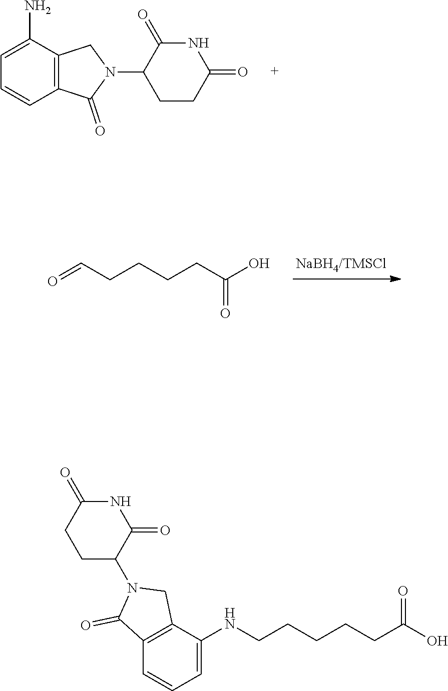 [SMILES: Nc1cccc2c1CN(C1CCC(=O)NC1=O)C2=O ; O=CCCCCC(=O)O ; O=C(O)CCCCCNc1cccc2c1CN(C1CCC(=O)NC1=O)C2=O]

To a solution of 3-(4-amino-1-oxoisoindolin-2-yl)piperidine-2,6-dione (30 mg, 0.12 mmol) and 6-oxohexanoic acid (45 mg, 0.35 mmol) in DMF (4 mL) were added NaBH₄ (8.8 mg, 0.23 mmol) and TMSCl (37.5 mg, 0.35 mmol) at 0° C. The reaction mixture was stirred at 0° C. for 4 h, and stirred at room temperature for 48 h. The reaction mixture was purified by prep-HPLC to give the desired product (16 mg, yield 37%) as a white solid. MS (ESI) m/z: 374.6 [M+H]⁺.

### Step 2: Synthesis of 3-(7-(difluoromethyl)-6-(1-methyl-1H-pyrazol-4-yl)-3,4-dihydroquinolin-1(2H)-yl)-1-(1-(6-((2-(2,6-dioxopiperidin-3-yl)-1-oxoisoindolin-4-yl)amino)hexanoyl)piperidin-4-yl)-N-methyl-1,4,6,7-tetrahydro-5H-pyrazolo[4,3-c]pyridine-5-carboxamide (P-095)

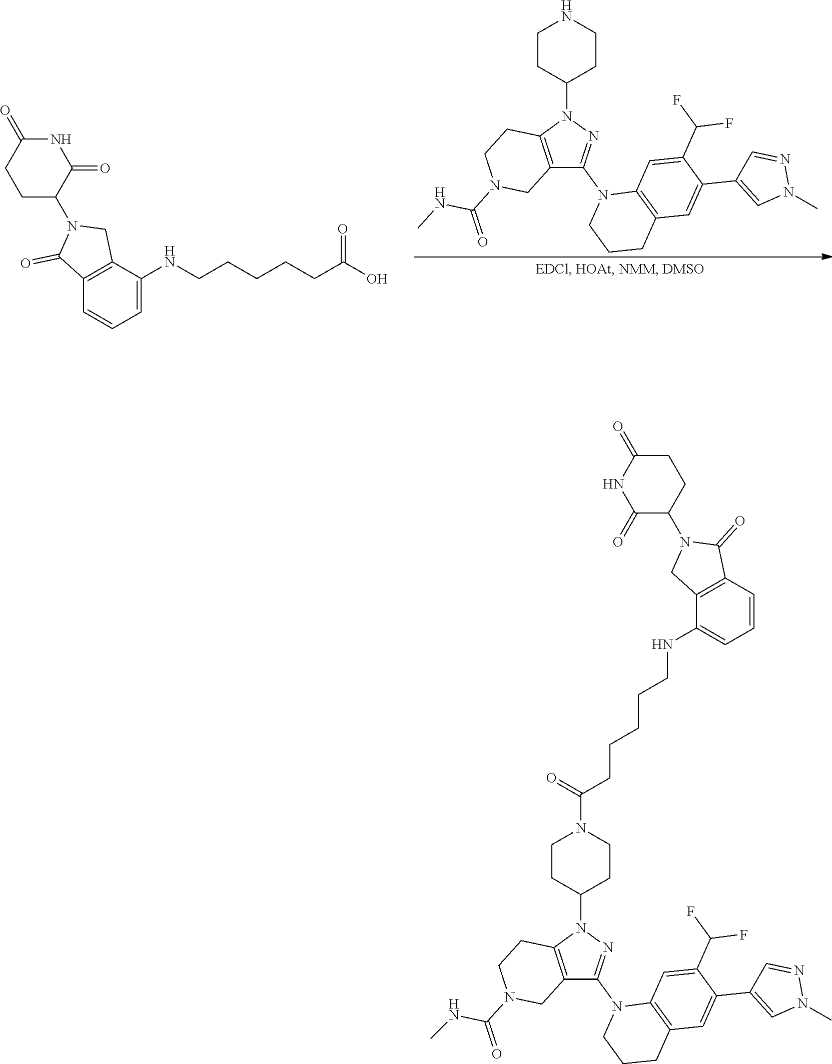

P-095 was synthesized following the standard procedure for preparing P-001 (3 mg, yield 26%). MS (ESI) m/z: 880.8 [M+H]⁺.

### Example 181. 3-(7-(Difluoromethyl)-6-(1-methyl-1H-pyrazol-4-yl)-3,4-dihydroquinolin-1(2H)-yl)-1-(1-(6-((2-(2,6-dioxopiperidin-3-yl)-3-oxoisoindolin-4-yl)amino)hexanoyl)piperidin-4-yl)-N-methyl-1,4,6,7-tetrahydro-5H-pyrazolo[4,3-c]pyridine-5-carboxamide (P-096)

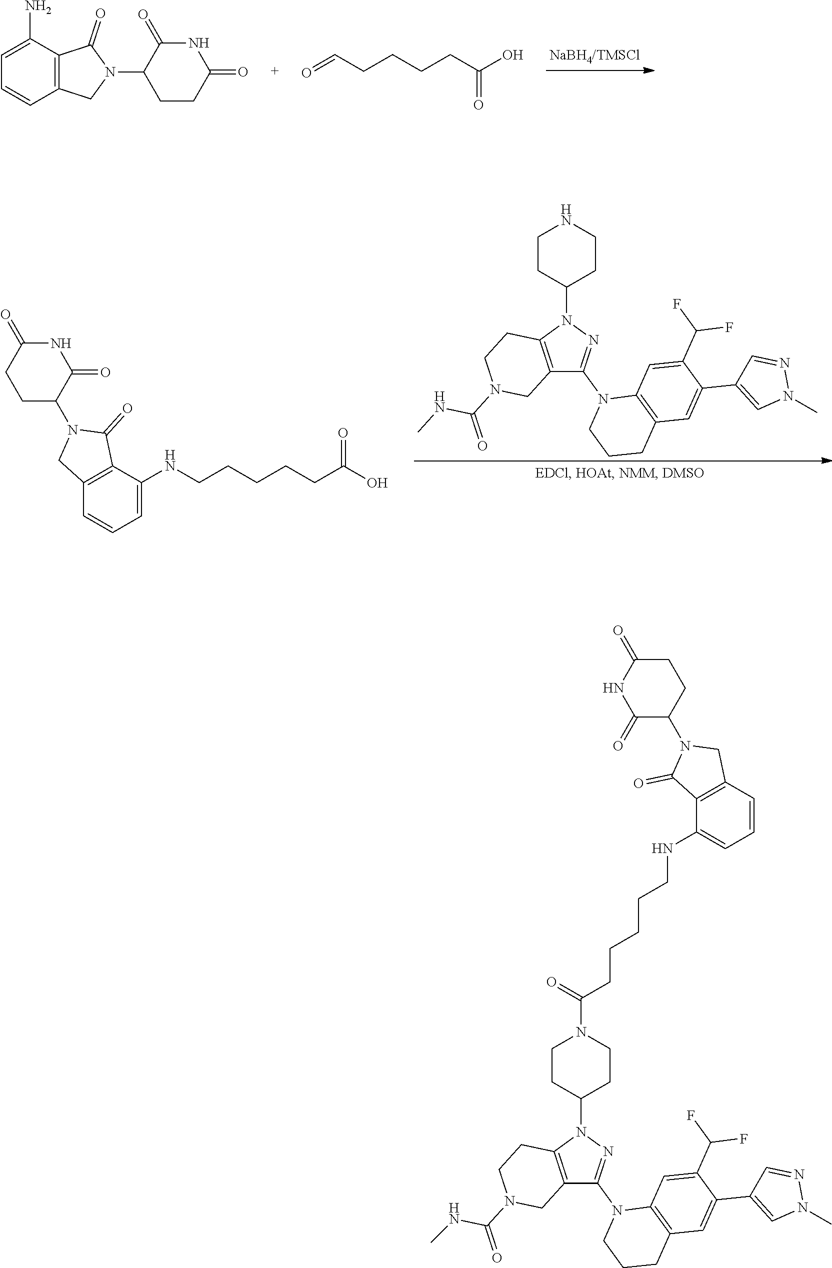

P-096 was synthesized following the similar procedure for preparing P-095 (2 mg, yield 24%). MS (ESI) m/z: 881.0 [M+H]⁺.

### Example 182. 3-(7-(Difluoromethyl)-6-(1-methyl-1H-pyrazol-4-yl)-3,4-dihydroquinolin-1(2H)-yl)-1-(1-(7-(2-(2,6-dioxopiperidin-3-yl)-1,3-dioxoisoindolin-4-yl)heptyl)piperidin-4-yl)-N-methyl-1,4,6,7-tetrahydro-5H-pyrazolo[4,3-c]pyridine-5-carboxamide (P-097)

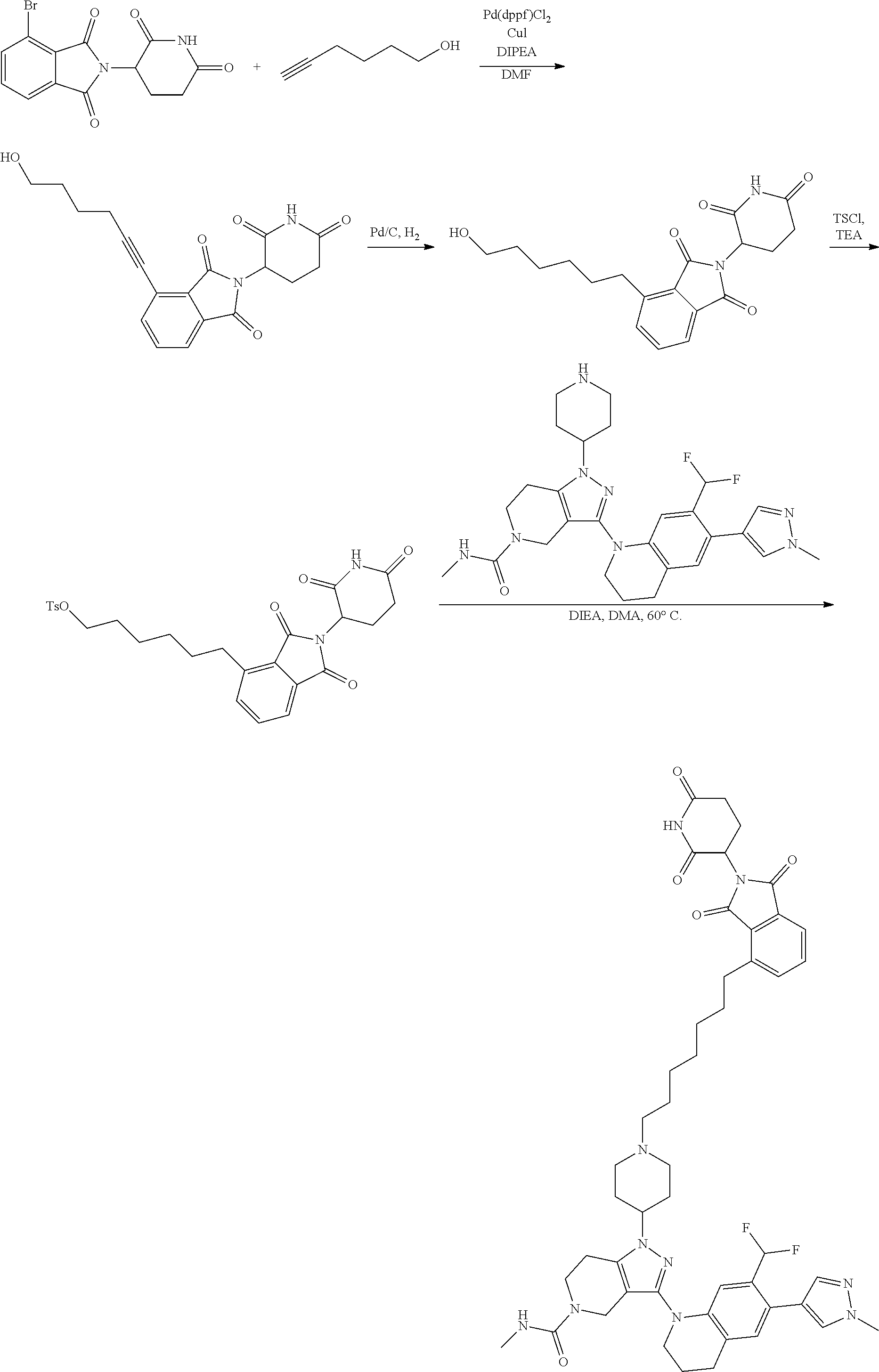

P-097 was synthesized following the similar procedure for preparing P-085 (1.08 mg, yield 11%). MS (ESI) m/z: 879.9 [M+H]⁺.

### Example 183. 3-(7-(Difluoromethyl)-6-(1-methyl-1H-pyrazol-4-yl)-3,4-dihydroquinolin-1(2H)-yl)-1-(1-(7-(2-(2,6-dioxopiperidin-3-yl)-1,3-dioxoisoindolin-4-yl)hept-6-yn-1-yl)piperidin-4-yl)-N-methyl-1,4,6,7-tetrahydro-5H-pyrazolo[4,3-c]pyridine-5-carboxamide (P-098)

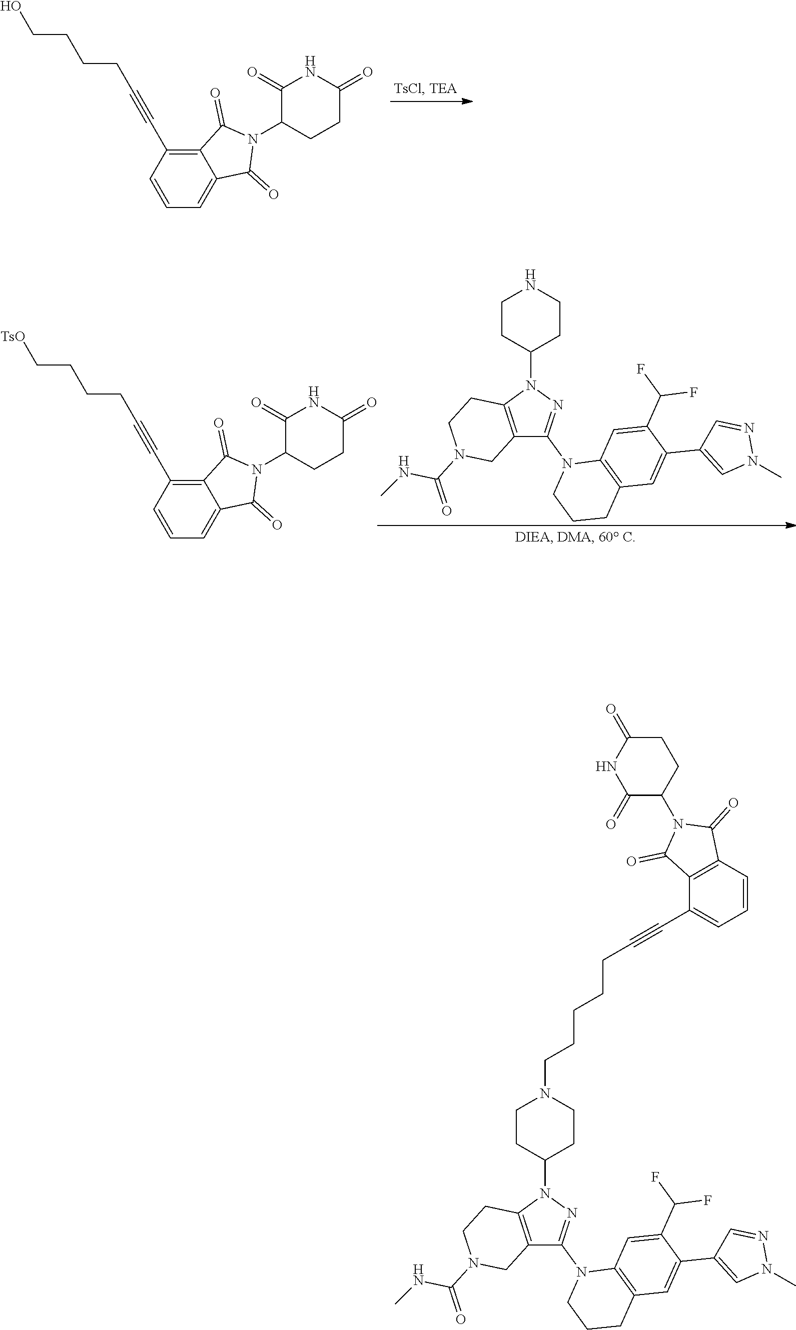

P-098 was synthesized following the similar procedure for preparing P-097 (1.01 mg, yield 12%). MS (ESI) m/z: 875.8 [M+H]⁺.

### Example 185. 4-((6-(4-(5-Acetyl-3-(7-(difluoromethyl)-6-(1-methyl-1H-pyrazol-4-yl)-3,4-dihydroquinolin-1(2H)-yl)-4,5,6,7-tetrahydro-1H-pyrazolo[4,3-c]pyridin-1-yl)piperidin-1-yl)-6-oxohexyl)amino)-2-(2,6-dioxopiperidin-3-yl)isoindoline-1,3-dione (P-100)

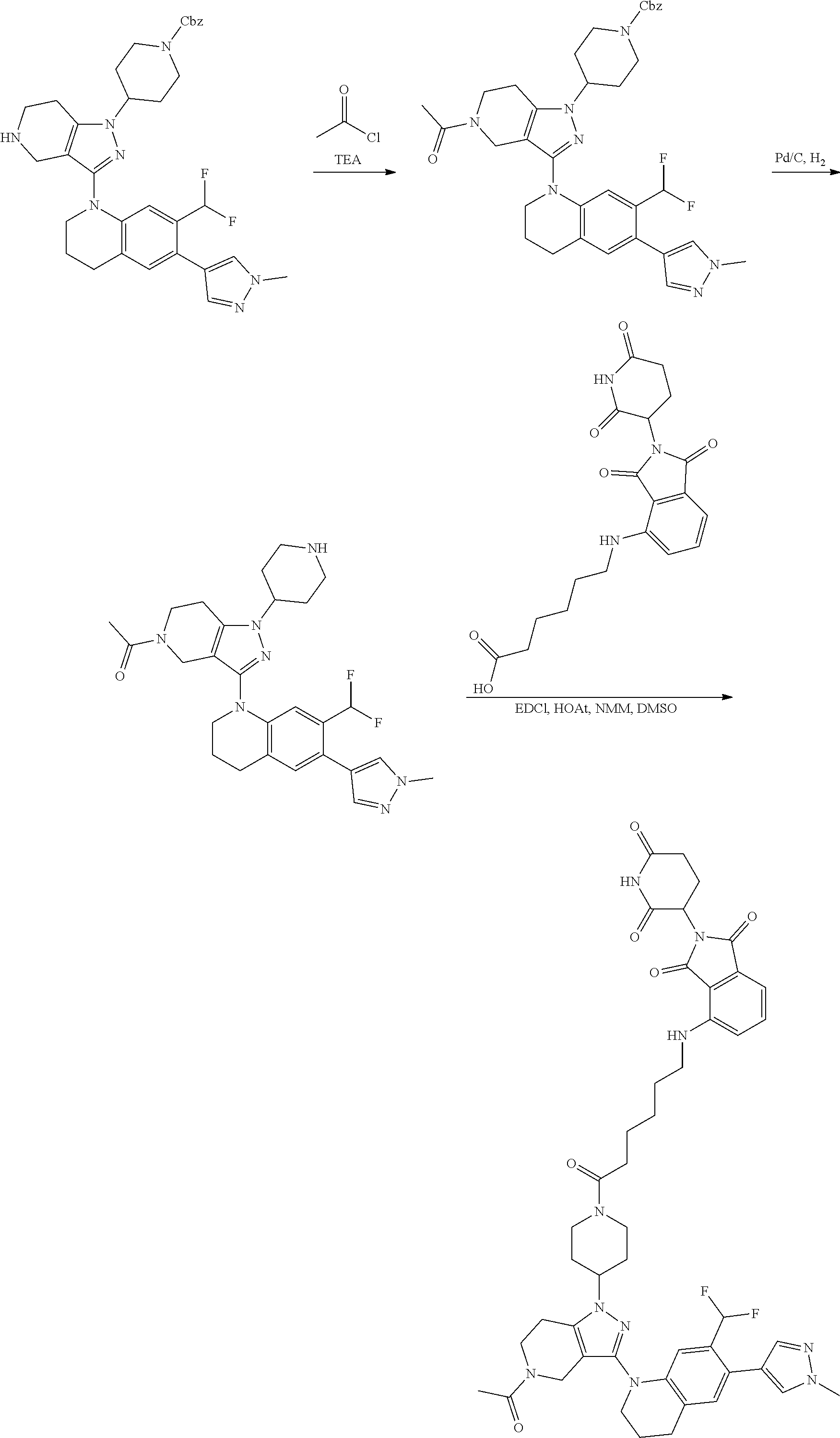

### Step 1: Synthesis of benzyl 4-(5-acetyl-3-(7-(difluoromethyl)-6-(1-methyl-1H-pyrazol-4-yl)-3,4-dihydroquinolin-1(2H)-yl)-4,5,6,7-tetrahydro-1H-pyrazolo[4,3-c]pyridin-1-yl)piperidine-1-carboxylate

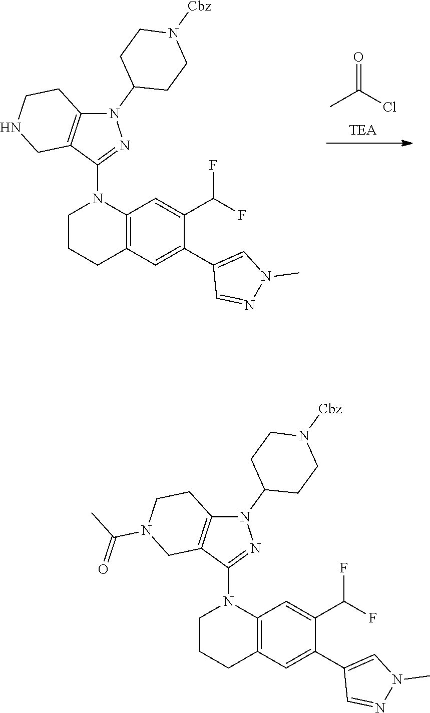

To a mixture of benzyl 4-(3-(7-(difluoromethyl)-6-(1-methyl-1H-pyrazol-4-yl)-3,4-dihydroquinolin-1(2Hs)-yl)-4,5,6,7-tetrahydro-1H-pyrazolo[4,3-c]pyridin-1-yl)piperidine-1-carboxylate (77 mg, 0.13 mmol) and TEA (39 mg, 0.38 mmol) in DCM (3 mL) was added a solution of acetyl chloride (15 mg, 0.19 mmol) in DCM (1 mL) dropwise at 0° C. The reaction mixture was stirred at 0° C. for 1.5 h, then it was concentrated. The residue was dissolved in MeOH and purified by prep-HPLC to give the desired product (51 mg, yield 62%). MS (ESI) m/z: 645.2 [M+H]⁺.

### Step 2: Synthesis of 1-(3-(7-(difluoromethyl)-6-(1-methyl-1H-pyrazol-4-yl)-3,4-dihydroquinolin-1(2H)-yl)-1-(piperidin-4-yl)-1,4,6,7-tetrahydro-5H-pyrazolo[4,3-c]pyridin-5-yl)ethan-1-one

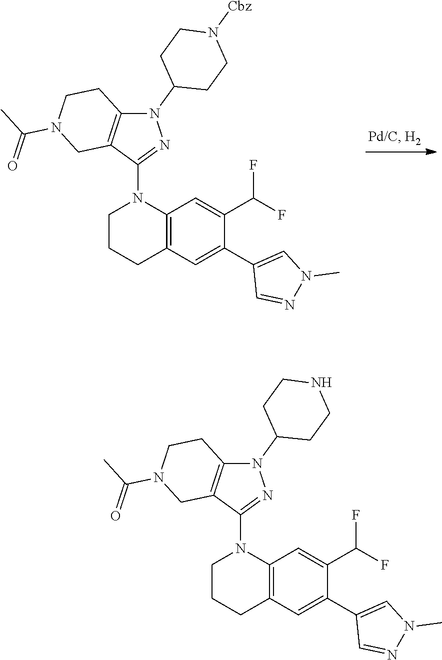

A mixture of benzyl 4-(5-acetyl-3-(7-(difluoromethyl)-6-(1-methyl-1H-pyrazol-4-yl)-3,4-dihydroquinolin-1(2H)-yl)-4,5,6,7-tetrahydro-1H-pyrazolo[4,3-c]pyridin-1-yl)piperidine-1-carboxylate (2.5 g, 3.88 mmol), Pd/C (231 mg) and TFA (one drop) in MeOH (24 mL) was stirred at 30° C. for 4 h, before the reaction mixture was filtered. After the filtrate was concentrated, the resulting residue was diluted with aq. NaHCO₃ and extracted with EtOAc. The combined organic layers were dried over Na₂SO₄, filtered and concentrated to give the desired product (1.8 g, yield 91%). MS (ESI) m/z: 511.0 [M+H]⁺.

### Step 3: Synthesis of 4-((6-(4-(5-acetyl-3-(7-(difluoromethyl)-6-(1-methyl-1H-pyrazol-4-yl)-3,4-dihydroquinolin-1(2H)-yl)-4,5,6,7-tetrahydro-1H-pyrazolo[4,3-c]pyridin-1-yl)piperidin-1-yl)-6-oxohexyl)amino)-2-(2,6-dioxopiperidin-3-yl)isoindoline-1,3-dione (P-100)

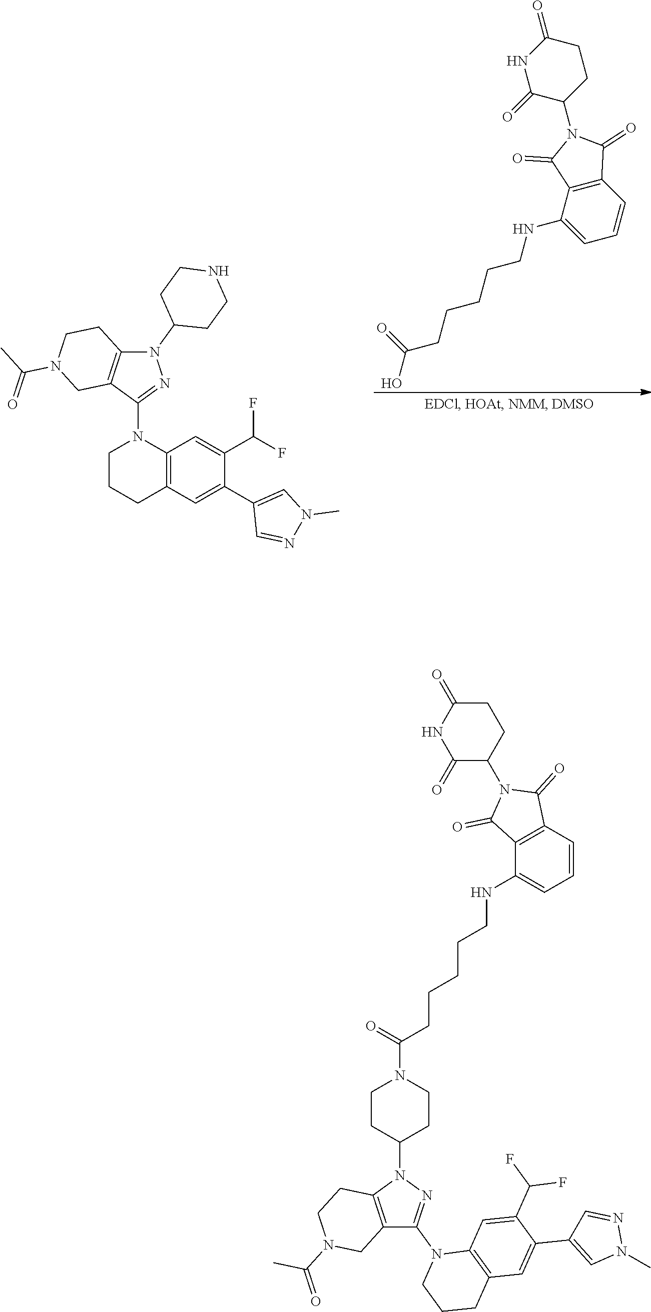 [SMILES: CC(=O)N1CCc2c(c(N3CCCc4cc(-c5cnn(C)c5)c(C(F)F)cc43)nn2C2CCNCC2)C1 ; O=C(O)CCCCCNc1cccc2c1C(=O)N(C1CCC(=O)NC1=O)C2=O ; CC(=O)N1CCc2c(c(N3CCCc4cc(-c5cnn(C)c5)c(C(F)F)cc43)nn2C2CCN(C(=O)CCCCCNc3cccc4c3C(=O)N(C3CCC(=O)NC3=O)C4=O)CC2)C1]

P-100 was synthesized following the standard procedure for preparing P-001 (8.7 mg, yield 63%). MS (ESI) m/z: 880.0 [M+H]⁺.

### Example 186. 3-(7-(Difluoromethyl)-6-(1-methyl-1H-pyrazol-4-yl)-3,4-dihydroquinolin-1(2H)-yl)-1-(1-(2-(4-((2-(2,6-dioxopiperidin-3-yl)-1,3-dioxoisoindolin-4-yl)amino)piperidin-1-yl)acetyl)piperidin-4-yl)-N-methyl-1,4,6,7-tetrahydro-5H-pyrazolo[4,3-c]pyridine-5-carboxamide (P-101)

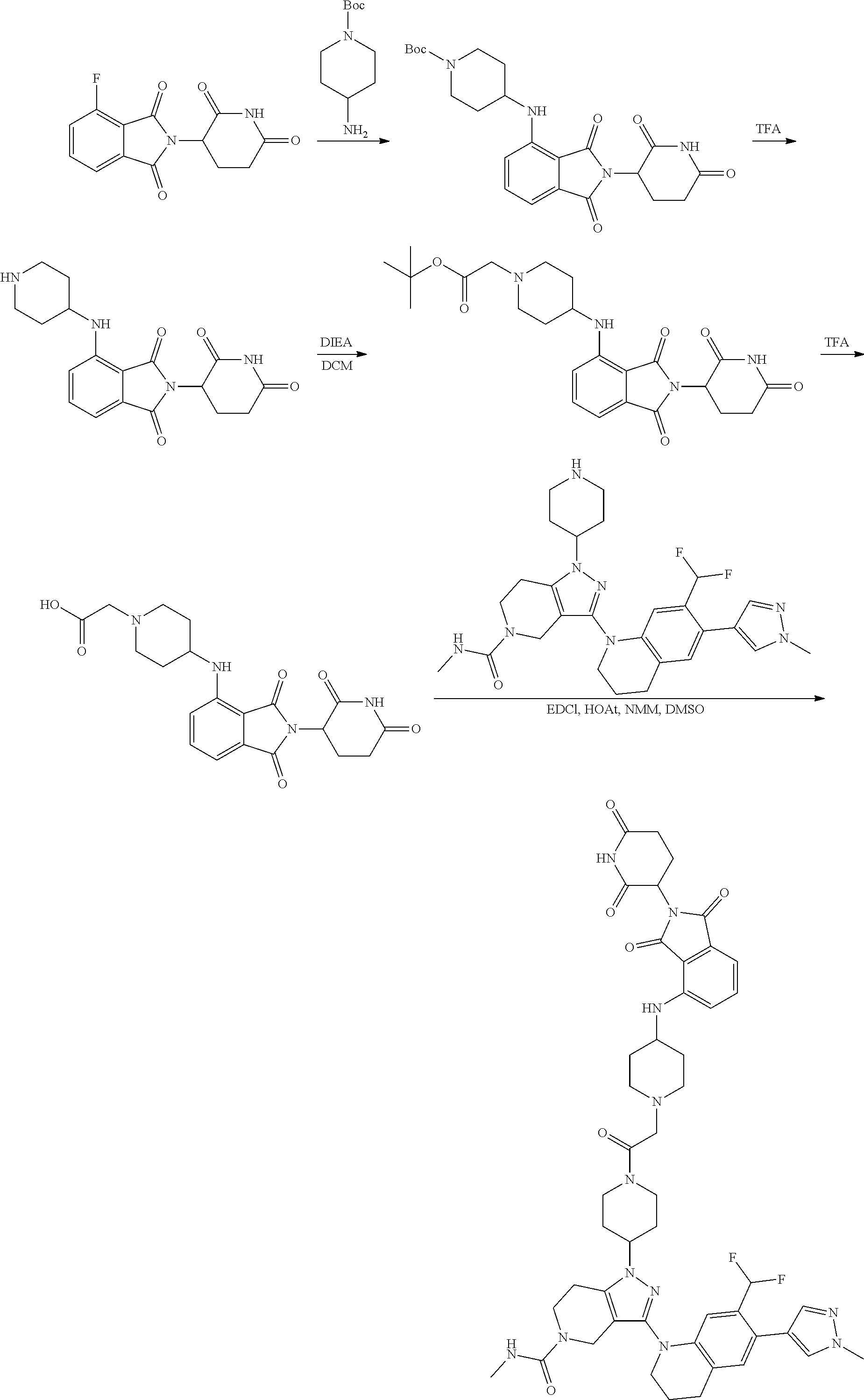

### Step 1: Synthesis of tert-butyl 4-((2-(2,6-dioxopiperidin-3-yl)-1,3-dioxoisoindolin-4-yl)amino)piperidine-1-carboxylate

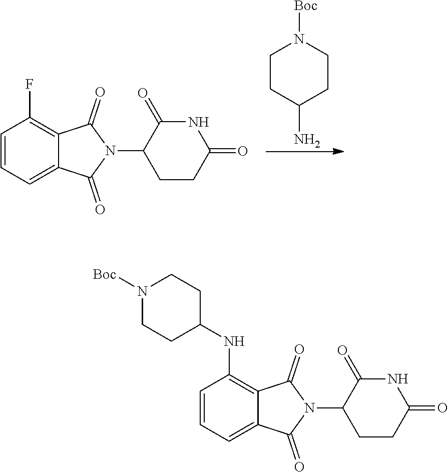 [SMILES: O=C1CCC(N2C(=O)c3cccc(F)c3C2=O)C(=O)N1 ; CC(C)(C)OC(=O)N1CCC(N)CC1 ; CC(C)(C)OC(=O)N1CCC(Nc2cccc3c2C(=O)N(C2CCC(=O)NC2=O)C3=O)CC1]

To a solution of 2-(2,6-dioxopiperidin-3-yl)-4-fluoroisoindoline-1,3-dione (2 g, 7.2 mmol) in DMSO (30 mL) were added KF (1.26 g, 21.7 mmol) and tert-butyl 4-aminopiperidine-1-carboxylate (4.34 g, 21.7 mmol). The resulting mixture was stirred at 130° C. for 1 h. After cooling to room temperature, the reaction mixture was poured into water and extracted with EtOAc. The combined organic layers were washed with brine, dried over Na₂SO₄, filtered and concentrated. The residue was purified with scilica gel column chromatography to give the desired product (1.2 g, yield 37%). MS (ESI) m/z: 457.4 [M+H]⁺.

### Step 2 to Step 5

P-101 was synthesized Following the similar procedure for preparing P-103 (6.2 mg, yield 71%). MS (ESI) m/z: 922.1 [M+H]⁺.

### Example 187. 3-(7-(Difluoromethyl)-6-(1-methyl-1H-pyrazol-4-yl)-3,4-dihydroquinolin-1(2H)-yl)-1-(1-(2-(4-(42-(2,6-dioxopiperidin-3-yl)-1,3-dioxoisoindolin-4-yl)amino)methyl)piperidin-1-yl)acetyl)piperidin-4-yl)-N-methyl-1,4,6,7-tetrahydro-5H-pyrazolo[4,3-c]pyridine-5-carboxamide (P-102)

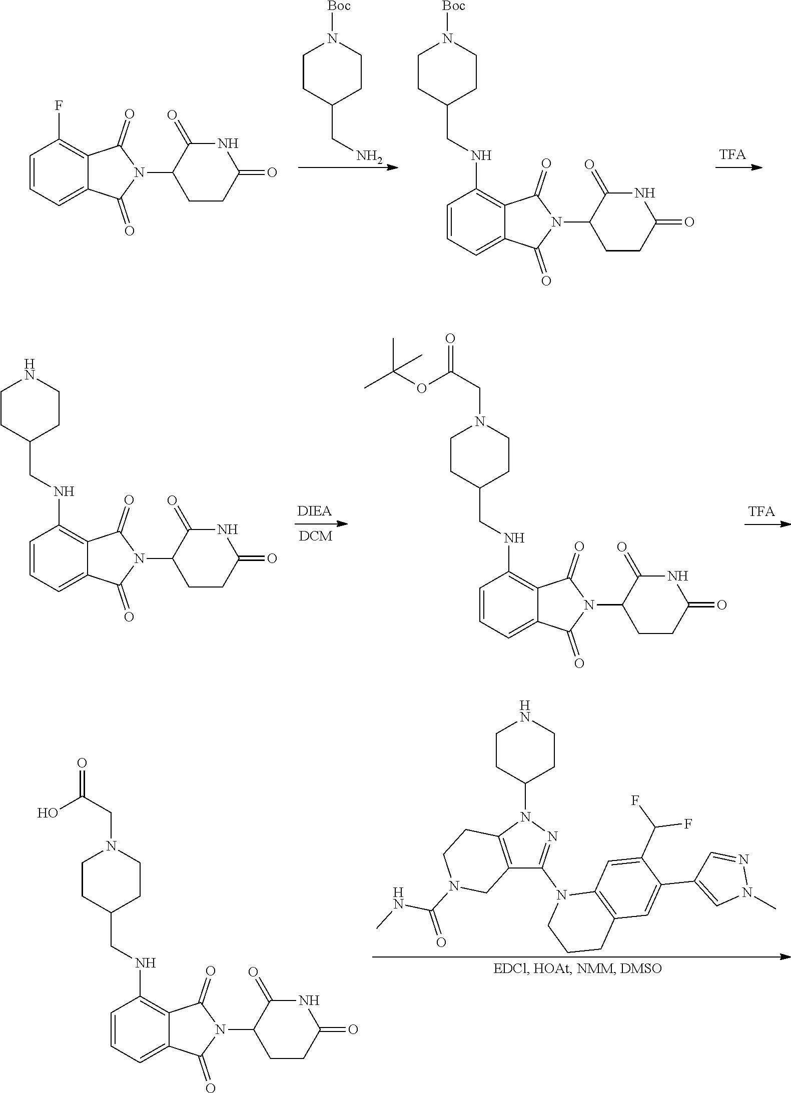

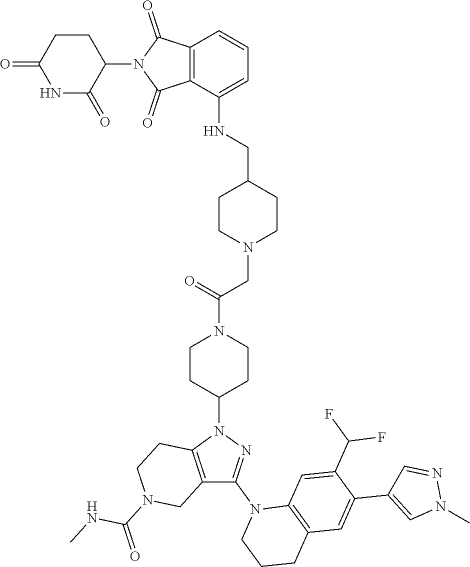 [SMILES: CNC(=O)N1CCc2c(c(N3CCCc4cc(-c5cnn(C)c5)c(C(F)F)cc43)nn2C2CCN(C(=O)CN3CCC(CNc4cccc5c4C(=O)N(C4CCC(=O)NC4=O)C5=O)CC3)CC2)C1]

P-102 was synthesized following the similar procedure for preparing P-101 (6.8 mg, yield 76%). MS (ESI) m/z: 936.1 [M+H]⁺.

### Example 188. 3-(7-(Difluoromethyl)-6-(1-methyl-1H-pyrazol-4-yl)-3,4-dihydroquinolin-1(2H)-yl)-1-(1-(2-(4-((2-(2,6-dioxopiperidin-3-yl)-1,3-dioxoisoindolin-4-yl)ethynyl)piperidin-1-yl)acetyl)piperidin-4-yl)-N-methyl-1,4,6,7-tetrahydro-5H-pyrazolo[4,3-c]pyridine-5-carboxamide (P-103)

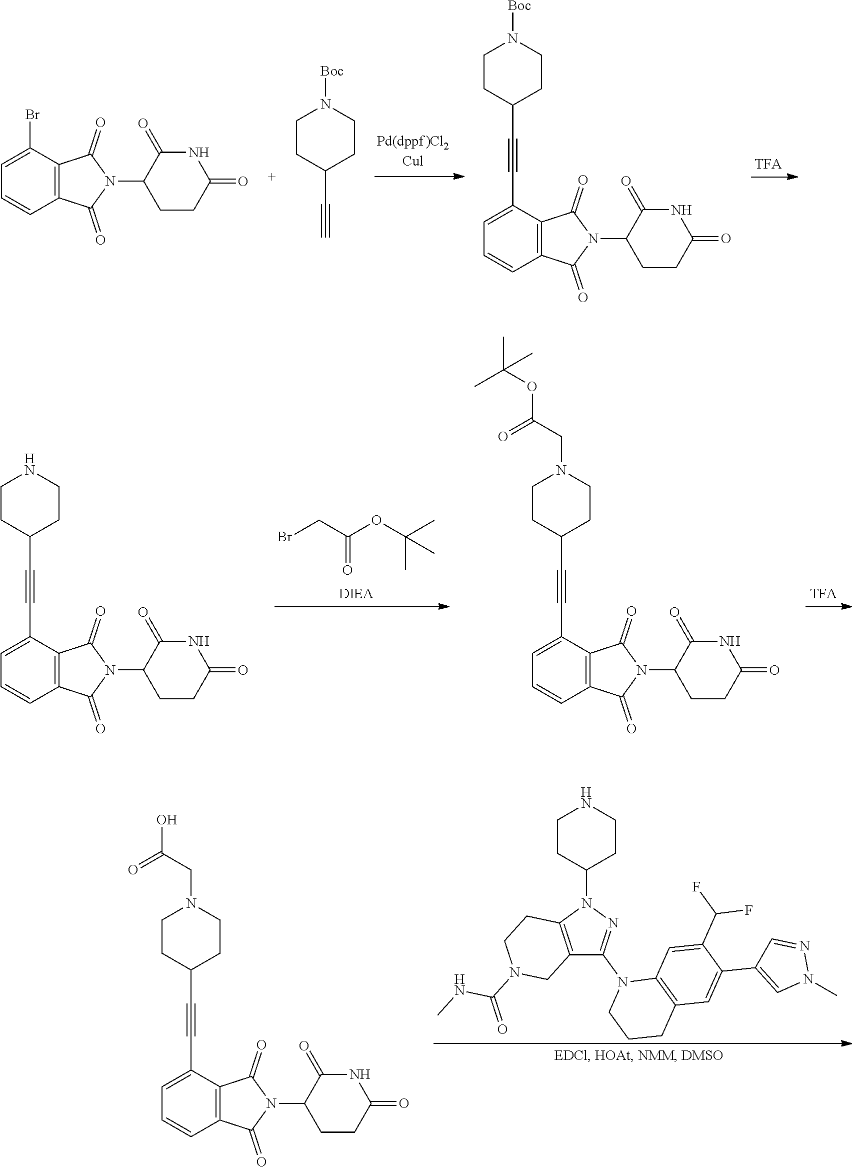

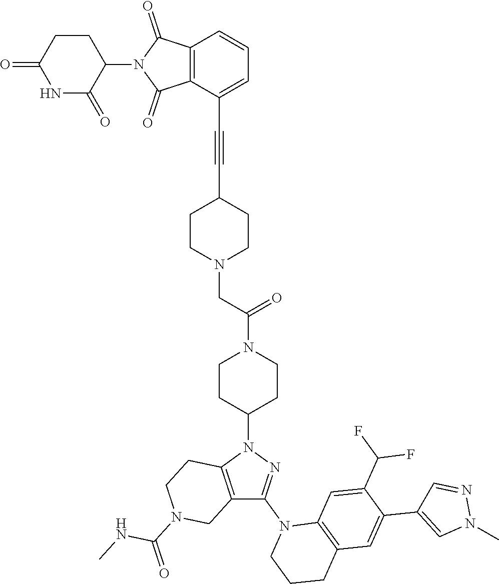 [SMILES: CNC(=O)N1CCc2c(c(N3CCCc4cc(-c5cnn(C)c5)c(C(F)F)cc43)nn2C2CCN(C(=O)CN3CCC(C#Cc4cccc5c4C(=O)N(C4CCC(=O)NC4=O)C5=O)CC3)CC2)C1]

### Step 1: Synthesis of tert-butyl 4-((2-(2,6-dioxopiperidin-3-yl)-1,3-dioxoisoindolin-4-yl)ethynyl)piperidine-1-carboxylate

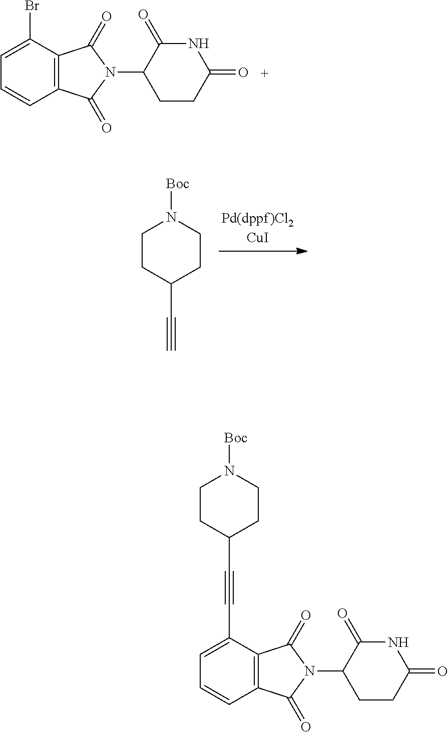 [SMILES: O=C1CCC(N2C(=O)c3cccc(Br)c3C2=O)C(=O)N1 ; C#CC1CCN(C(=O)OC(C)(C)C)CC1 ; CC(C)(C)OC(=O)N1CCC(C#Cc2cccc3c2C(=O)N(C2CCC(=O)NC2=O)C3=O)CC1]

To a solution of 4-bromo-2-(2,6-dioxopiperidin-3-yl)isoindoline-1,3-dione (300 mg, 0.89 mmol) in DMF (5 mL) were added tert-butyl 4-ethynylpiperidine-1-carboxylate (205 mg, 0.98 mmol), Pd(dppf)Cl₂ (32.5 mg, 0.044 mmol), CuI (17 mg, 0.089 mmol) and TEA (899 mg, 8.9 mmol). The resulting mixture was stirred at 80° C. for 16 h under N₂. The reaction mixture was purified by reverse phase chromatography to give the desired product (260 mg, yield 63%). MS (ESI) ink: 410.5 [M-^{t}Bu+H]⁺.

### Step 2: Synthesis of 2-(2,6-dioxopiperidin-3-yl)-4-(piperidin-4-ylethynyl)isoindoline-1,3-dione

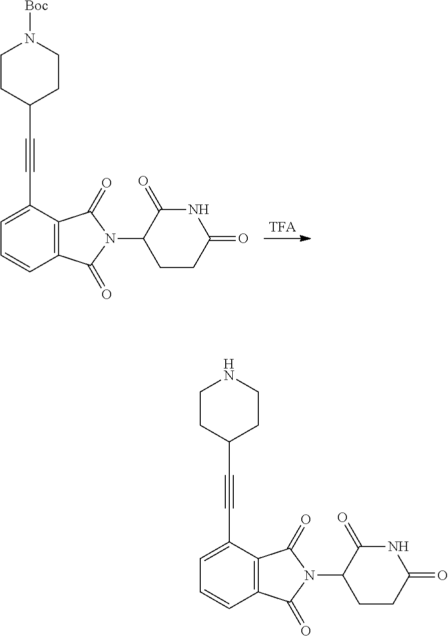 [SMILES: CC(C)(C)OC(=O)N1CCC(C#Cc2cccc3c2C(=O)N(C2CCC(=O)NC2=O)C3=O)CC1 ; O=C1CCC(N2C(=O)c3cccc(C#CC4CCNCC4)c3C2=O)C(=O)N1]

A mixture of tert-butyl 4-((2-(2,6-dioxopiperidin-3-yl)-1,3-dioxoisoindolin-4-yl)ethynyl)piperidine-1-carboxylate (260 mg, 0.56 mmol) in DCM (10 mL) and TFA (1 mL) was stirred at room temperature for 1.5 h. The reaction mixture was concentrated to give the crude product, which was used directly in the next step without further purification.

### Step 3: Synthesis of tert-butyl 2-(4-((2-(2,6-dioxopiperidin-3-yl)-1,3-dioxoisoindolin-4-yl)ethynyl)piperidin-1-yl)acetate

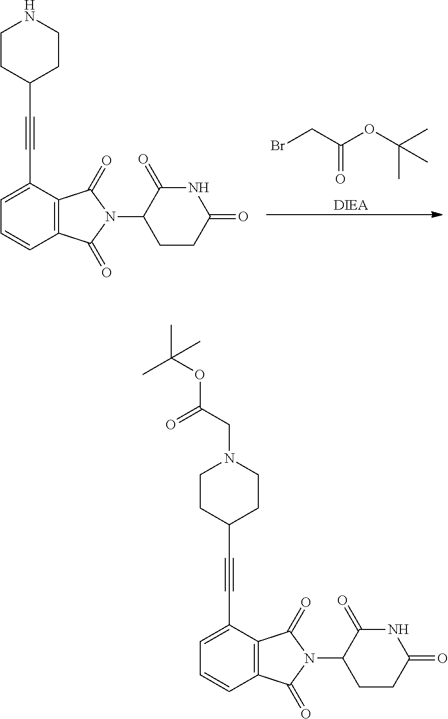

A mixture of 2-(2,6-dioxopiperidin-3-yl)-4-(piperidin-4-ylethynyl)isoindoline-1,3-dione (181 mg, 0.5 mmol), tert-butyl 2-bromoacetate (145 mg, 0.74 mmol) and DIEA (319 mg, 2.48 mmol) in DMF (5 mL) was stirred at 30° C. for 2 h. The reaction mixture was poured into water and extracted with EtOAc. The combined organic layers were dried over Na₂SO₄, filtered and concentrated. The residue was purified by silica gel column chromatography (Petroleum ether/EtOAc=1:1) to give the desired product (237 mg, yield 99%). MS (ESI) m/z: 480.9 [M+H]⁺.

### Step 4: Synthesis of 2-(4-((2-(2,6-dioxopiperidin-3-yl)-1,3-dioxoisoindolin-4-yl)ethynyl)piperidin-1-yl)acetic acid

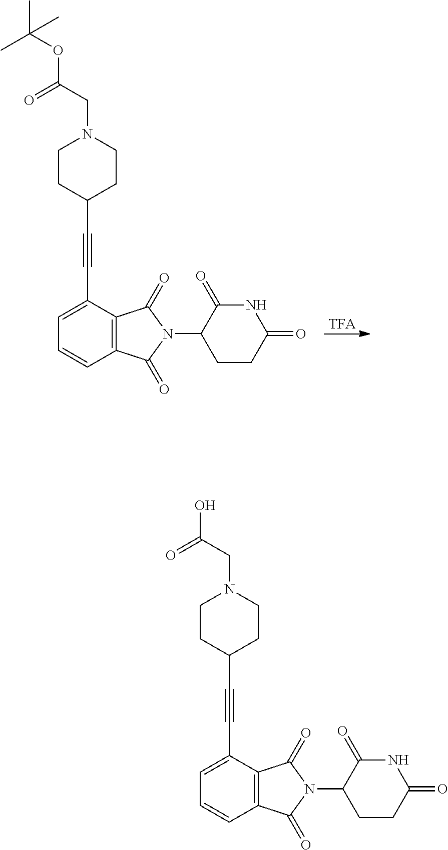 [SMILES: CC(C)(C)OC(=O)CN1CCC(C#Cc2cccc3c2C(=O)N(C2CCC(=O)NC2=O)C3=O)CC1 ; O=C(O)CN1CCC(C#Cc2cccc3c2C(=O)N(C2CCC(=O)NC2=O)C3=O)CC1]

A mixture of tert-butyl 2-(4-((2-(2,6-dioxopiperidin-3-yl)-1,3-dioxoisoindolin-4-yl)ethynyl)piperidin-1-yl)acetate (237 mg, 0.49 mmol) in DCM (3 mL) and TFA (1 mL) was stirred at room temperature for 1.5 h. The reaction mixture was concentrated to give the desired product, which was used directly in the next step without further purification.

### Step 4: Synthesis of 3-(7-(difluoromethyl)-6-(1-methyl-1H-pyrazol-4-yl)-3,4-dihydroquinolin-1(2H)-yl)-1-(1-(2-(4-((2-(2,6-dioxopiperidin-3-yl)-1,3-dioxoisoindolin-4-yl)ethynyl)piperidin-1-yl)acetyl)piperidin-4-yl)-N-methyl-1,4,6,7-tetrahydro-5H-pyrazolo[4,3-c]pyridine-5-carboxamide (P-103)

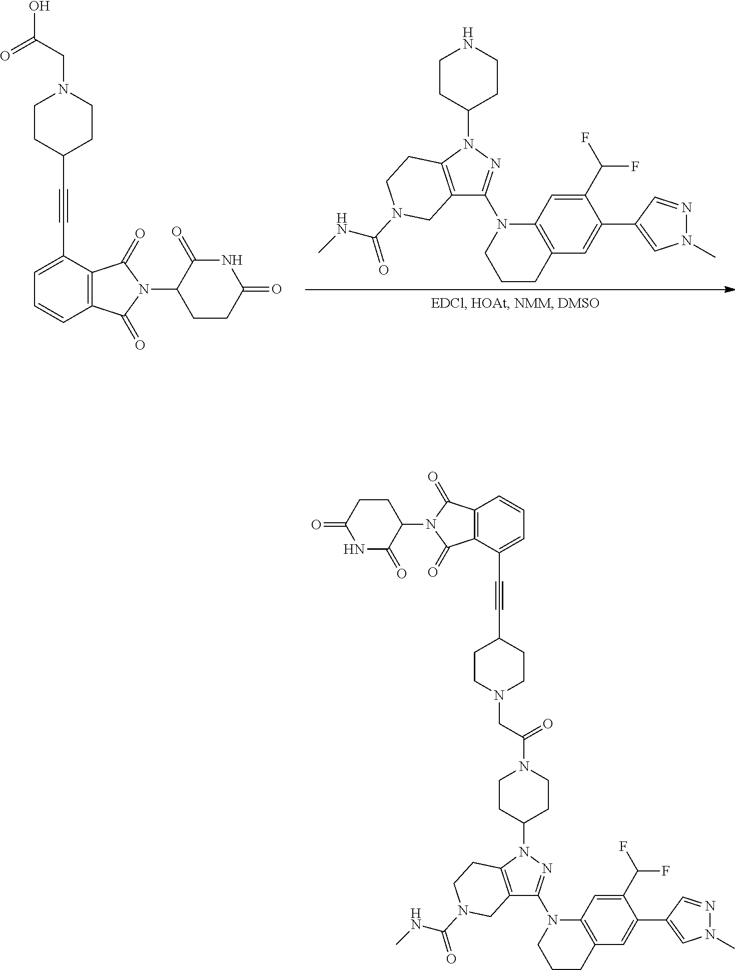

P-103 was synthesized following the standard procedure for preparing P-001 (9.3 mg, yield 75%). MS (ESI) m/z: 931.0 [M+H]⁺.

### Example 189. 3-(7-(Difluoromethyl)-6-(1-methyl-1H-pyrazol-4-yl)-3,4-dihydroquinolin-1(2H)-yl)-1-(1-(2-(4-(2-(2-(2,6-dioxopiperidin-3-yl)-1,3-dioxoisoindolin-4-yl)ethyl)piperidin-1-yl)acetyl)piperidin-4-yl)-N-methyl-1,4,6,7-tetrahydro-5H-pyrazolo[4,3-c]pyridine-5-carboxamide (P-104)

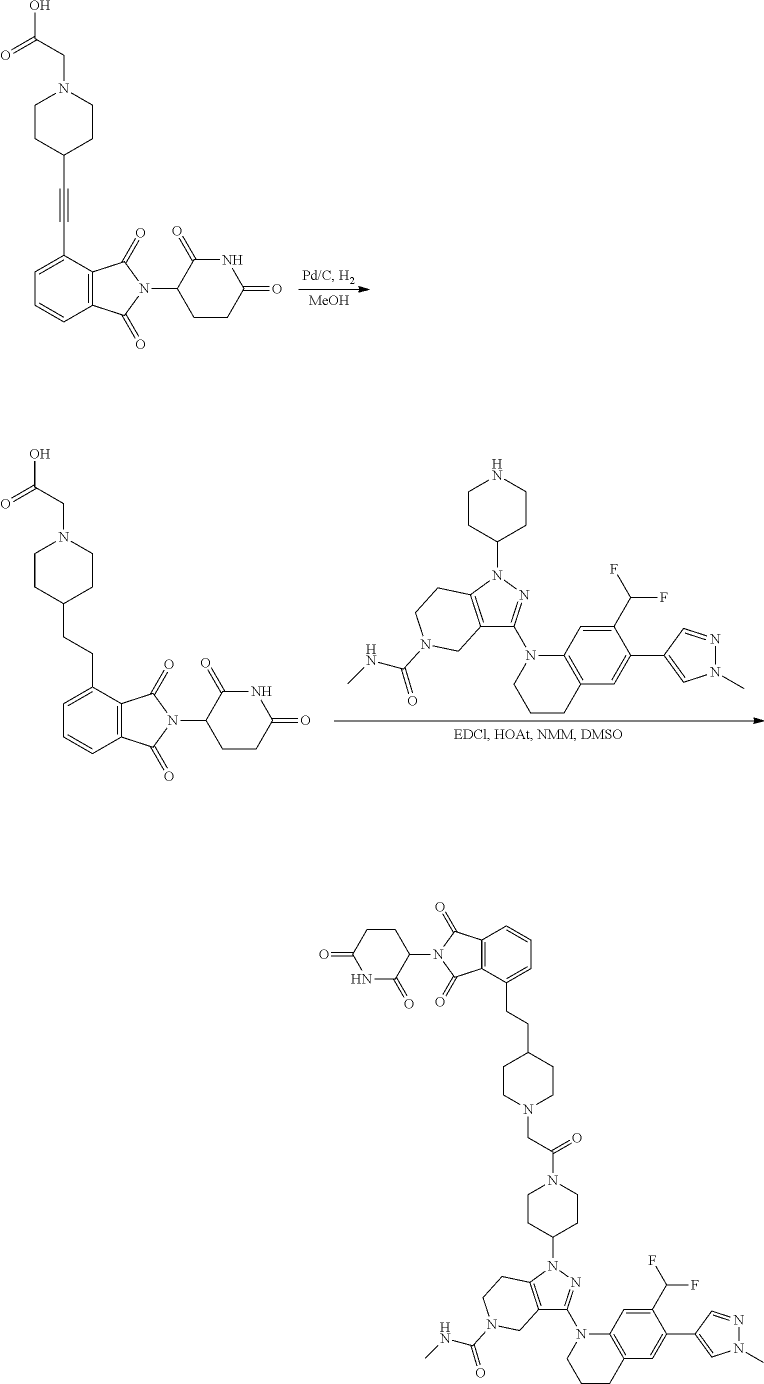

P-104 was synthesized following the similar procedure for preparing P-085 (7.0 mg, yield 32%). MS (ESI) m/z: 935.0 [M+H]⁺.

### Example 190. 3-(7-(Difluoromethyl)-6-(1-methyl-1H-pyrazol-4-yl)-3,4-dihydroquinolin-1(2H)-yl)-1-(1-(6-(3-(2,6-dioxopiperidin-3-yl)-2-oxo-2,3-dihydro-1H-benzo[d]imidazol-1-yl)hexanoyl)piperidin-4-yl)-N-methyl-1,4,6,7-tetrahydro-5H-pyrazolo[4,3-c]pyridine-5-carboxamide (P-105)

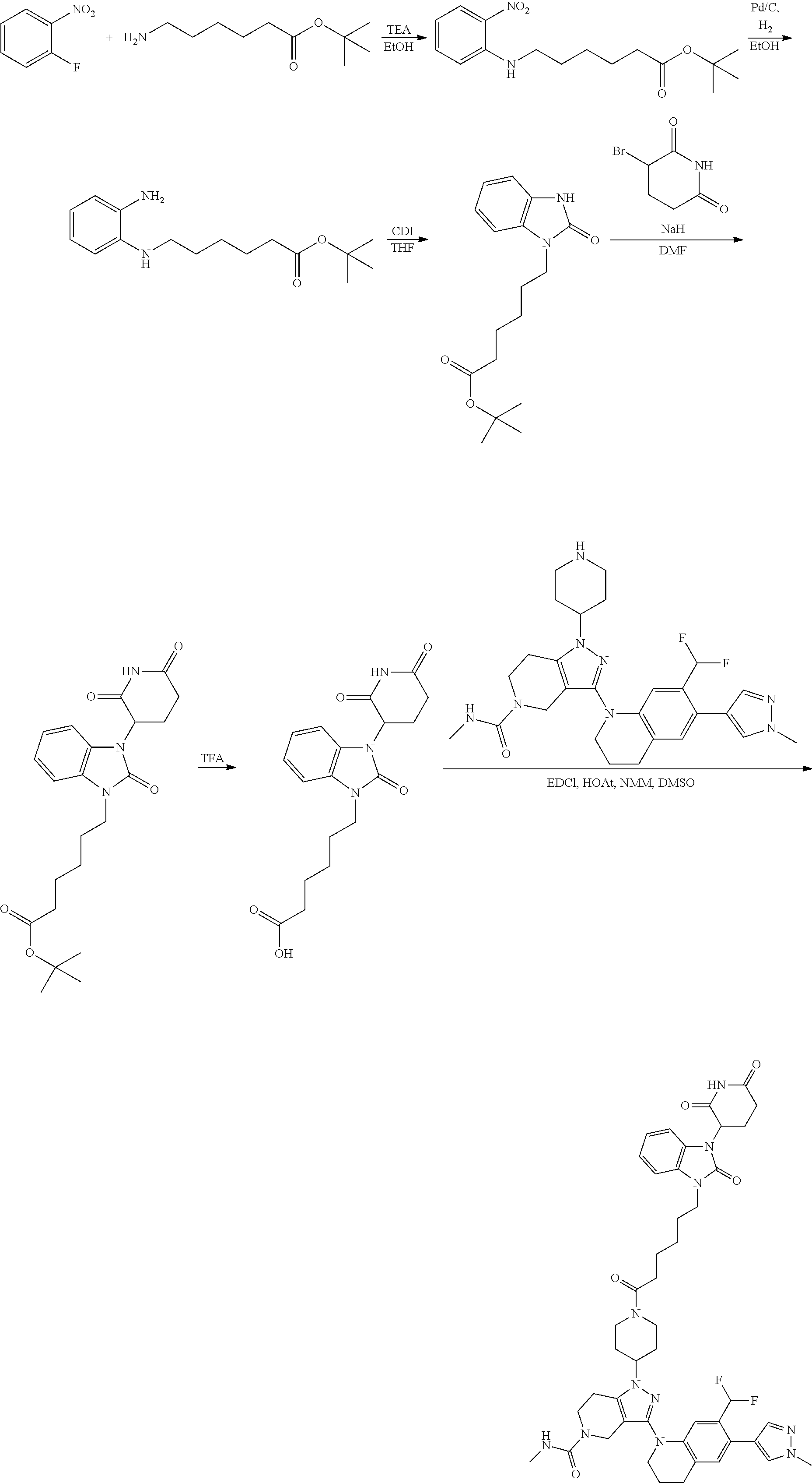

### Step 1: Synthesis of tert-butyl 6-((2-nitrophenyl)amino)hexanoate

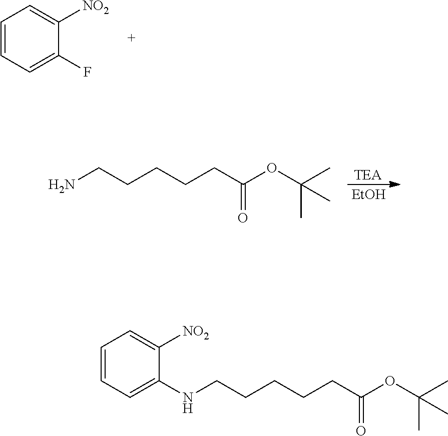 [SMILES: "EtOH" ; O=[N+]([O-])c1ccccc1F ; CC(C)(C)OC(=O)CCCCCN ; CC(C)(C)OC(=O)CCCCCNc1ccccc1[N+](=O)[O-]]

A mixture of 1-fluoro-2-nitrobenzene (528 mg, 3.74 mmol), tert-butyl 6-aminohexanoate (841 mg, 4.49 mmol) and TEA (1.14 g, 11.23 mmol) in EtOH (10 mL) was stirred at 85° C. overnight. The reaction mixture was concentrated to afford the crude product, which was used directly in the next step without further purification.

### Step 2: Synthesis of tert-butyl 6-((2-aminophenyl)amino)hexanoate

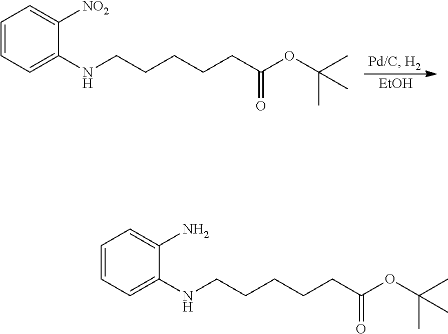 [SMILES: "EtOH" ; CC(C)(C)OC(=O)CCCCCNc1ccccc1[N+](=O)[O-] ; CC(C)(C)OC(=O)CCCCCNc1ccccc1N]

A mixture of tert-butyl 6-((2-nitrophenyl)amino)hexanoate (1.2 g, 3.89 mmol), Pd/C (100 mg) in EtOH (30 mL) was stirred under H₂ at room temperature for 1.5 h. After the reaction mixture was filtered through a Celite cup, the filtrate was concentrated. The resulting residue was purified by silica gel column chromatography (Petroleum ether/EtOAc=8:1 to 5:1) to give the desired product (690 mg, yield 64%). (ESI) m/z: 279.7 [M+H]⁺.

### Step 3: Synthesis of tert-butyl 6-(2-oxo-2,3-dihydro-1H-benzo[d]imidazol-1-yl)hexanoate

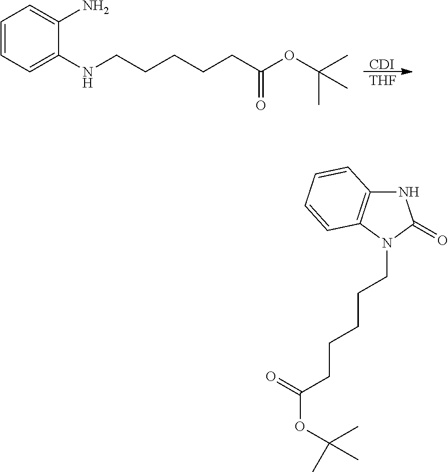

To a solution of tert-butyl 6-((2-aminophenyl)amino)hexanoate (592 mg, 2.13 mmol) in THF (40 mL) was added CDI (517 mg, 3.19 mmol). The resulting mixture was stirred at room temperature for 16 h, before the reaction mixture was concentrated and diluted with EtOAc, and washed with water. The organic layer was dried over Na₂SO₄, filtered and concentrated. The residue was purified by silica gel column chromatography (Petroleum ether/EtOAc=3:1 to 2:1) to give the desired product (579 mg, yield 89%). (ESI) m/z: 305.5 [M+H]⁺.

### Step 4: Synthesis of tert-butyl 6-(3-(2,6-dioxopiperidin-3-yl)-2-oxo-2,3-dihydro-1H-benzo[d]imidazol-1-yl)hexanoate

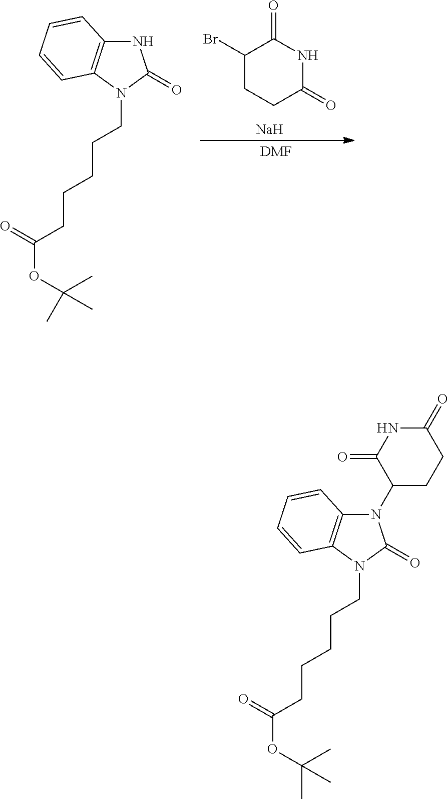

To a solution of tert-butyl 6-(2-oxo-2,3-dihydro-1H-benzo[d]imidazol-1-yl)hexanoate (550 mg, 1.81 mmol) in anhydrous DMF (2.2 ml) was added NaH (56 mg, 2.35 mmol) at 0° C. under N2. The resulting mixture was stirred at 0° C. for 15 min, before a solution of 3-bromopiperidine-2,6-dione (173 mg, 0.90 mmol) in anhydrous DMF (2.2 mL) was added dropwise over 10 min. After the reaction mixture was stirred at room temperature overnight, it was quenched with water and extracted with EtOAc. The aqueous phase was adjusted to pH=5-6 with citric acid and extracted with EtOAc. The combined organic layers were dried over Na₂SO₄, filtered and concentrated. The residue was purified by silica gel column chromatography (Petroleum ether/EtOAc=2:1 to 1:1) to give desired product (170 mg, yield 23%). (ESI) m/z: 416.6 [M+H]⁺.

### Step 5: Synthesis of 6-(3-(2,6-dioxopiperidin-3-yl)-2-oxo-2,3-dihydro-1H-benzo[d]imidazol-1-yl)hexanoic acid

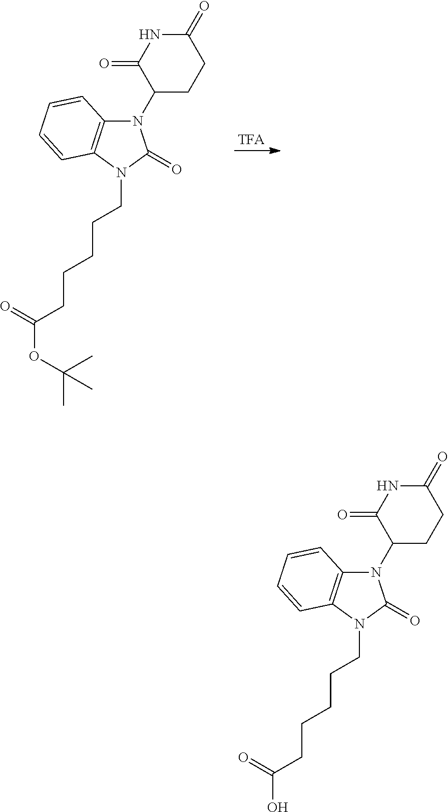 [SMILES: CC(C)(C)OC(=O)CCCCCn1c(=O)n(C2CCC(=O)NC2=O)c2ccccc21 ; O=C(O)CCCCCn1c(=O)n(C2CCC(=O)NC2=O)c2ccccc21]

A mixture of tert-butyl 6-(3-(2,6-dioxopiperidin-3-yl)-2-oxo-2,3-dihydro-1H-benzo[d]imidazol-1-yl)hexanoate (170 mg, 0.41 mmol) in DCM (3 mL) and TFA (3 mL) was stirred at room temperature for 0.5 h. The solvent was removed to afford the desired product (147 mg, yield 99%). (ESI) m/z: 360.6 [M+H]⁺.

### Step 6: Synthesis of 3-(7-(difluoromethyl)-6-(1-methyl-1H-pyrazol-4-yl)-3,4-dihydroquinolin-1(2H)-yl)-1-(1-(6-(3-(2,6-dioxopiperidin-3-yl)-2-oxo-2,3-dihydro-1H-benzo[d]imidazol-1-yl)hexanoyl)piperidin-4-yl)-N-methyl-1,4,6,7-tetrahydro-5H-pyrazolo[4,3-c]pyridine-5-carboxamide (P-105)

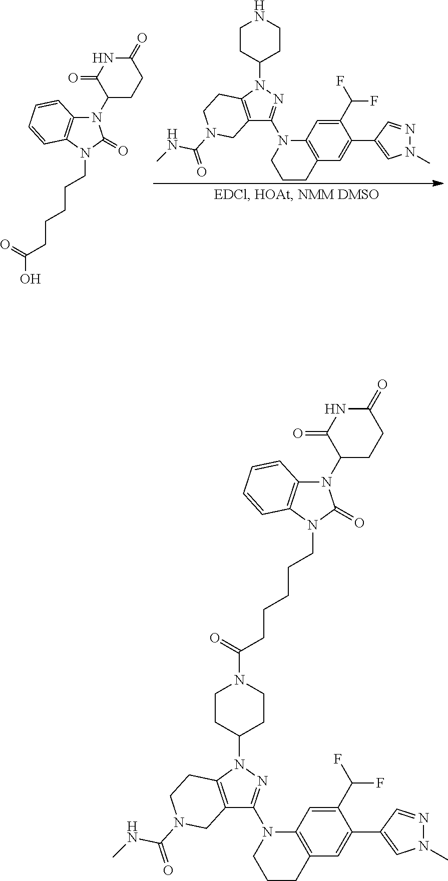

P-105 was synthesized following the standard procedure for preparing P-001 (10.8 mg, yield 65%). MS (ESI) m/z: 867.1 [M+H]⁺.

### Example 191. 3-(7-(Difluoromethyl)-6-(1-methyl-1H-pyrazol-4-yl)-3,4-dihydroquinolin-1(2H)-yl)-1-(1-(7-(3-(2,6-dioxopiperidin-3-yl)-2-oxo-2,3-dihydro-1H-benzo[d]imidazol-1-yl)heptanoyl)piperidin-4-yl)-N-methyl-1,4,6,7-tetrahydro-5H-pyrazolo[4,3-c]pyridine-5-carboxamide (P-106)

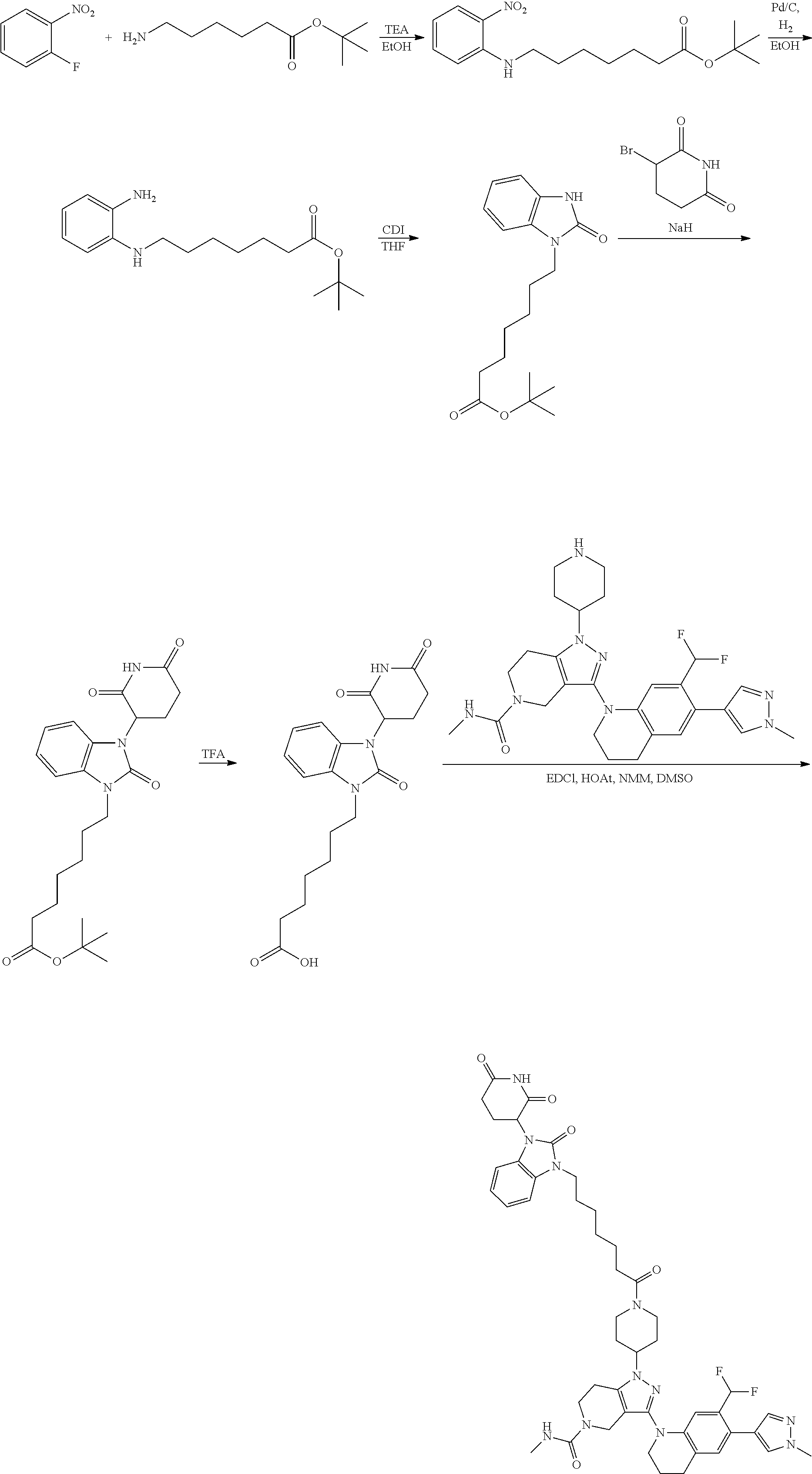

P-106 was synthesized following the similar procedure for preparing P-105 (13.4 mg, yield 80%). MS (ESI) m/z: 881.2 [M+H]⁺.

### Example 192. 3-(3-(6-(4-(5-Acetyl-3-(7-(difluoromethyl)-6-(1-methyl-1H-pyrazol-4-yl)-3,4-dihydroquinolin-1(2H)-yl)-4,5,6,7-tetrahydro-1H-pyrazolo[4,3-c]pyridin-1-yl)piperidin-1-yl)-6-oxohexyl)-2-oxo-2,3-dihydro-1H-benzo[d]imidazol-1-yl)piperidine-2,6-dione (P-107)

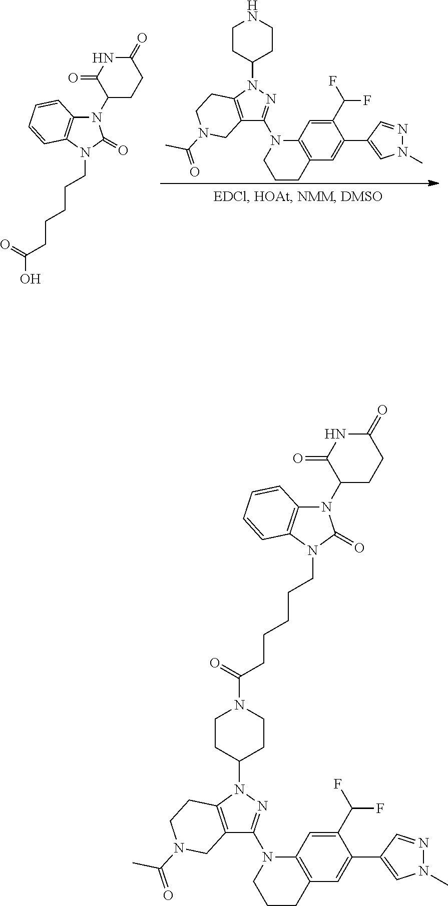 [SMILES: O=C(O)CCCCCn1c(=O)n(C2CCC(=O)NC2=O)c2ccccc21 ; CC(=O)N1CCc2c(c(N3CCCc4cc(-c5cnn(C)c5)c(C(F)F)cc43)nn2C2CCNCC2)C1 ; CC(=O)N1CCc2c(c(N3CCCc4cc(-c5cnn(C)c5)c(C(F)F)cc43)nn2C2CCN(C(=O)CCCCCn3c(=O)n(C4CCC(=O)NC4=O)c4ccccc43)CC2)C1]

P-107 was synthesized following the standard procedure for preparing P-001 (8.4 mg, yield 50%). MS (ESI) m/z: 852.1 [M+H]⁺.

### Example 193. 3-(3-(7-(4-(5-Acetyl-3-(7-(difluoromethyl)-6-(1-methyl-1H-pyrazol-4-yl)-3,4-dihydroquinolin-1(2H)-yl)-4,5,6,7-tetrahydro-1H-pyrazolo[4,3-c]pyridin-1-yl)piperidin-1-yl)-7-oxoheptyl)-2-oxo-2,3-dihydro-1H-benzo[d]imidazol-1-yl)piperidine-2,6-dione (P-108)

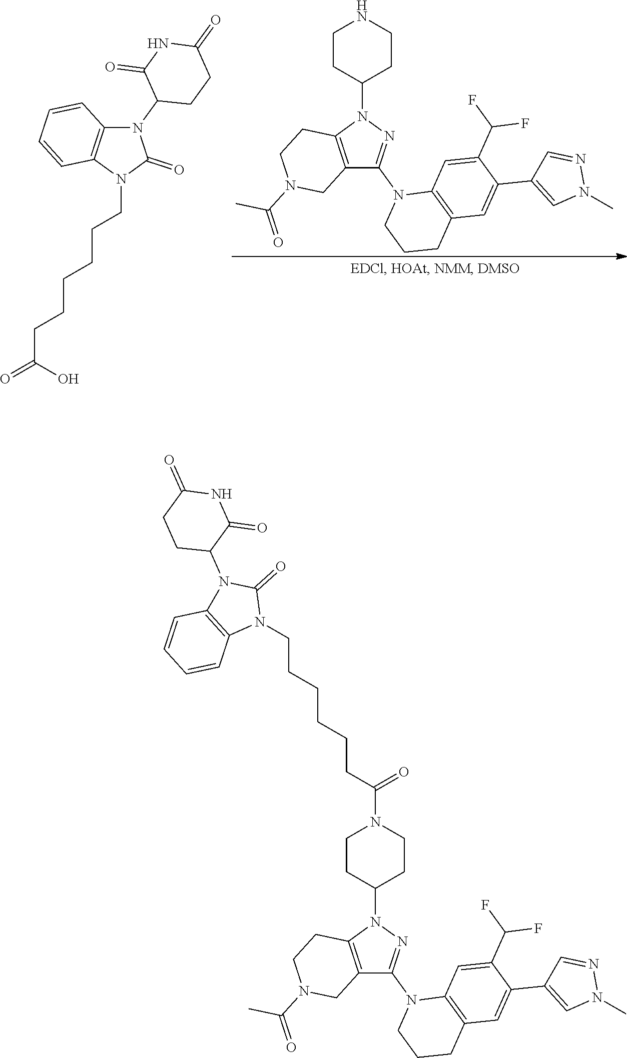 [SMILES: O=C(O)CCCCCCn1c(=O)n(C2CCC(=O)NC2=O)c2ccccc21 ; CC(=O)N1CCc2c(c(N3CCCc4cc(-c5cnn(C)c5)c(C(F)F)cc43)nn2C2CCNCC2)C1 ; CC(=O)N1CCc2c(c(N3CCCc4cc(-c5cnn(C)c5)c(C(F)F)cc43)nn2C2CCN(C(=O)CCCCCCn3c(=O)n(C4CCC(=O)NC4=O)c4ccccc43)CC2)C1]

P-108 was synthesized following the standard procedure for preparing P-001 (9.7 mg, yield 57%). MS (ESI) m/z: 866.2 [M+H]⁺.

### Example 194. 3-(7-(Difluoromethyl)-6-(1-methyl-1H-pyrazol-4-yl)-3,4-dihydroquinolin-1(2H)-yl)-1-(1-(6-((1-(2,6-dioxopiperidin-3-yl)-3-methyl-2-oxo-2,3-dihydro-1H-benzo[d]imidazol-4-yl)amino)hexanoyl)piperidin-4-yl)-N-methyl-1,4,6,7-tetrahydro-5H-pyrazolo[4,3-c]pyridine-5-carboxamide (P-109)

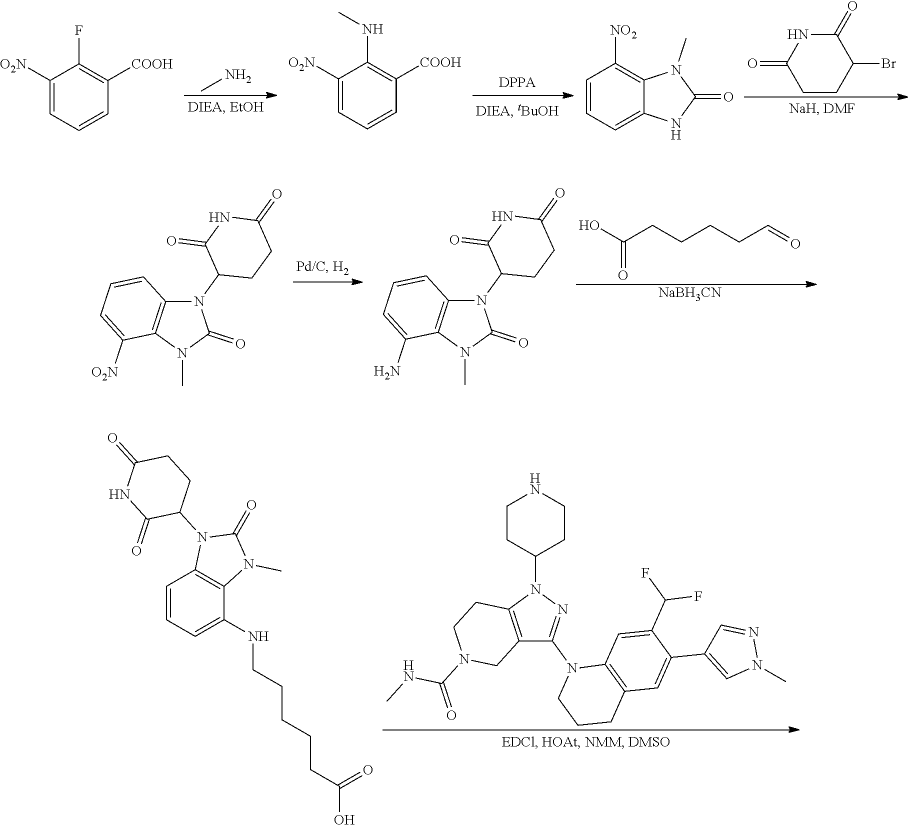

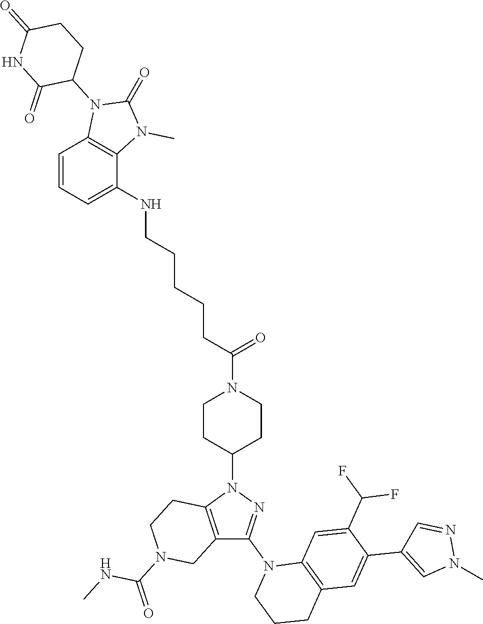 [SMILES: CNC(=O)N1CCc2c(c(N3CCCc4cc(-c5cnn(C)c5)c(C(F)F)cc43)nn2C2CCN(C(=O)CCCCCNc3cccc4c3n(C)c(=O)n4C3CCC(=O)NC3=O)CC2)C1]

### Step 1: Synthesis of 2-(methylamino)-3-nitrobenzoic acid

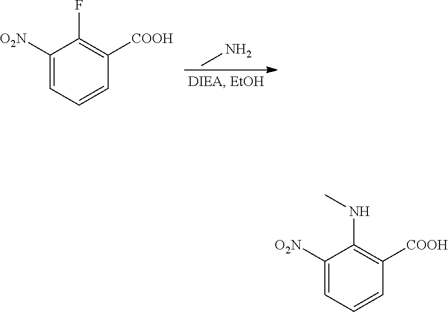

A mixture of 2-fluoro-3-nitrobenzoic acid (20 g, 108 mmol), methylamine hydrochloride (36.47 g, 540 mmol) and DIEA (167.25 g, 1.30 mol) in EtOH (300 mL) was stirred at 80° C. for 2 h. The reaction was concentrated and the residue was poured into ice water. The pH was adjusted to -3 with aq. HCl. The aqueous phase was extracted with EtOAc (3×). The combined organic layers were washed with brine, dried over Na₂SO₄, filtered and concentrated to give the desired product (24 g, yield 99%) as a yellow solid which was used directly in the next step.

### Step 2: Synthesis of 1-methyl-7-nitro-1,3-dihydro-2H-benzo[d]imidazol-2-one

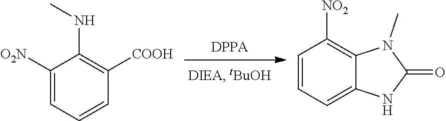

A solution of 2-(methylamino)-3-nitrobenzoic acid (24 g, 122.35 mmol), DPPA (35.71 g, 146.82 mmol) and DIEA (31.63 g, 244.70 mmol) in ^{t}BuOH (250 mL) was stirred at 90° C. overnight. After the reaction mixture was concentrated, the residue was poured into ice water. The precipitate was collected by filtration. The collected solid material was washed with water followed by EtOAc, dried in vacuum to give the desired product (22 g, yield 93%) as a yellow solid. (ESI) m/z: 194.1 [M+H]⁺.

### Step 3: Synthesis of 3-(3-methyl-4-nitro-2-oxo-2,3-dihydro-1H-benzo[d]imidazol-1-yl)piperidine-2,6-dione

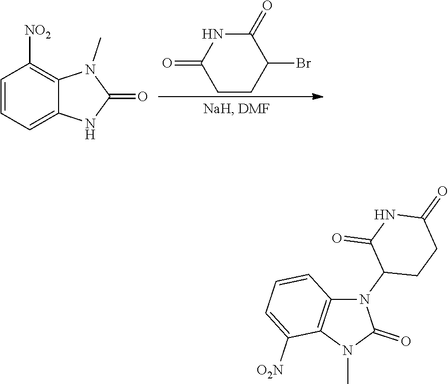

To a suspension of NaH (48 mg, 1.20 mmol) in DMF (5 mL) was added 3-methyl-5-nitro-1H-benzimidazol-2-one (193 mg, 999.18 umol) at 0° C. The reaction mixture was stirred at 0° C. for 0.5 h, before a solution of 3-bromopiperidine-2,6-dione (383.70 mg, 2.00 mmol) in DMF (5 mL) was added dropwise. After the completion of addition, the reaction mixture was stirred at 80° C. for 2 h. After concentration, the reaction was purified by prep-HPLC to give the desired product (80 mg, yield 26%) as a black solid. MS (ESI) m/z: 305.3 [M+H]⁺.

### Step 4: Synthesis of 3-(4-amino-3-methyl-2-oxo-2,3-dihydro-1H-benzo[d]imidazol-1-yl)piperidine-2,6-dione

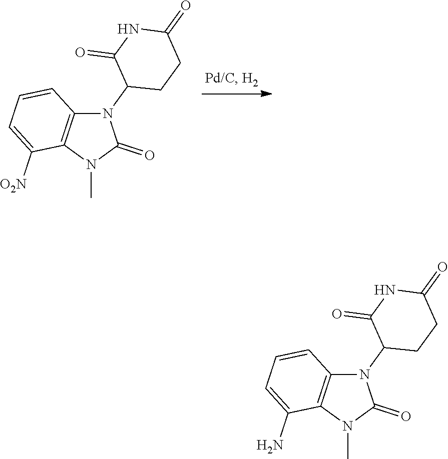 [SMILES: Cn1c(=O)n(C2CCC(=O)NC2=O)c2cccc([N+](=O)[O-])c21 ; Cn1c(=O)n(C2CCC(=O)NC2=O)c2cccc(N)c21]

To a solution of 3-(3-methyl-4-nitro-2-oxo-benzimidazol-1-yl)piperidine-2,6-dione (80 mg, 262.93 umol) in MeOH (10 mL) and THF (10 mL) was added Pd/C (20 mg) at room temperature. After the reaction mixture was stirred at room temperature for 1 h under hydrogen atmosphere, the reaction was filtered and concentrated to give the desired product (70 mg, yield 97%) as a brown solid, which was used in the next step directly without further purification. MS (ESI) m/z: 275.3 [M+H]⁺.

### Step 5: Synthesis of 6-((1-(2,6-dioxopiperidin-3-yl)-3-methyl-2-oxo-2,3-dihydro-1H-benzo[d]imidazol-4-yl)amino)hexanoic acid

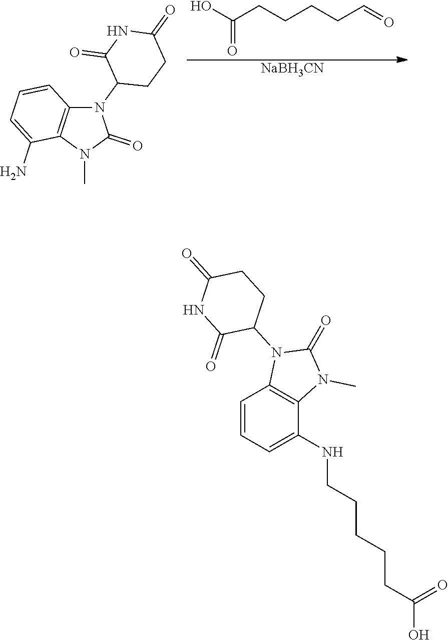

A mixture of 3-(4-amino-3-methyl-2-oxo-2,3-dihydro-1H-benzo[d]imidazol-1-yl)piperidine-2,6-dione (5 mg, 0.018 mmol) and 6-oxohexanoic acid (4.74 mg, 0.036 mmol) in ⁱPrOH (2 mL) and AcOH (2 mL) was stirred at 90° C. for 4 h. After the reaction was cooled to room temperature, NaBH₃CN (2.3 mg, 0.036 mmol) was added. The reaction mixture was stirred at room temperature for 2 h, before it was concentrated. The residue was purified by prep-TLC (DCM/MeOH=10:1) to give desired product (3 mg, yield 42%) as a white solid. MS (ESI) m/z: 389.7 [M+H]⁺.

### Step 6: Synthesis of 3-(7-(difluoromethyl)-6-(1-methyl-1H-pyrazol-4-yl)-3,4-dihydroquinolin-1(2H)-yl)-1-(1-(6-((1-(2,6-dioxopiperidin-3-yl)-3-methyl-2-oxo-2,3-dihydro-1H-benzo[d]imidazol-4-yl)amino)hexanoyl)piperidin-4-yl)-N-methyl-1,4,6,7-tetrahydro-5H-pyrazolo[4,3-c]pyridine-5-carboxamide (P-109)

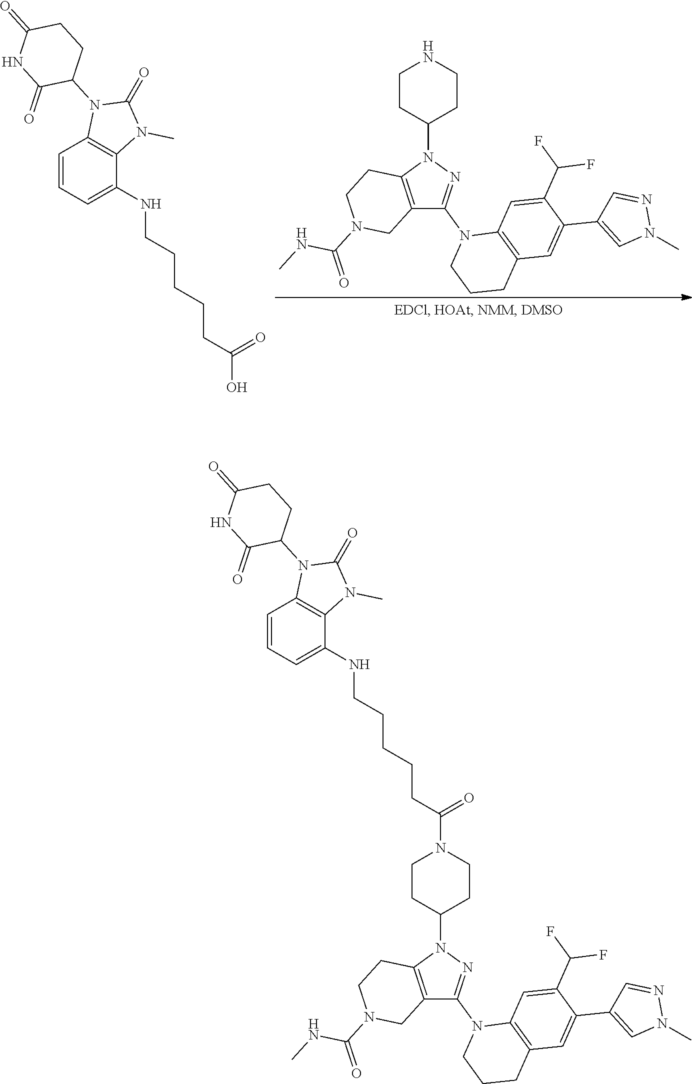

P-109 was synthesized following the standard procedure for preparing P-001 (1.5 mg, yield 16%). MS (ESI) m/z: 896.2 [M+H]⁺.

### Example 195. 3-(4-((6-(4-(5-Acetyl-3-(7-(difluoromethyl)-6-(1-methyl-1H-pyrazol-4-yl)-3,4-dihydroquinolin-1(2H)-yl)-4,5,6,7-tetrahydro-1H-pyrazolo[4,3-c]pyridin-1-yl)piperidin-1-yl)-6-oxohexyl)amino)-3-methyl-2-oxo-2,3-dihydro-1H-benzo[d]imidazol-1-yl)piperidine-2,6-dione (P-110)

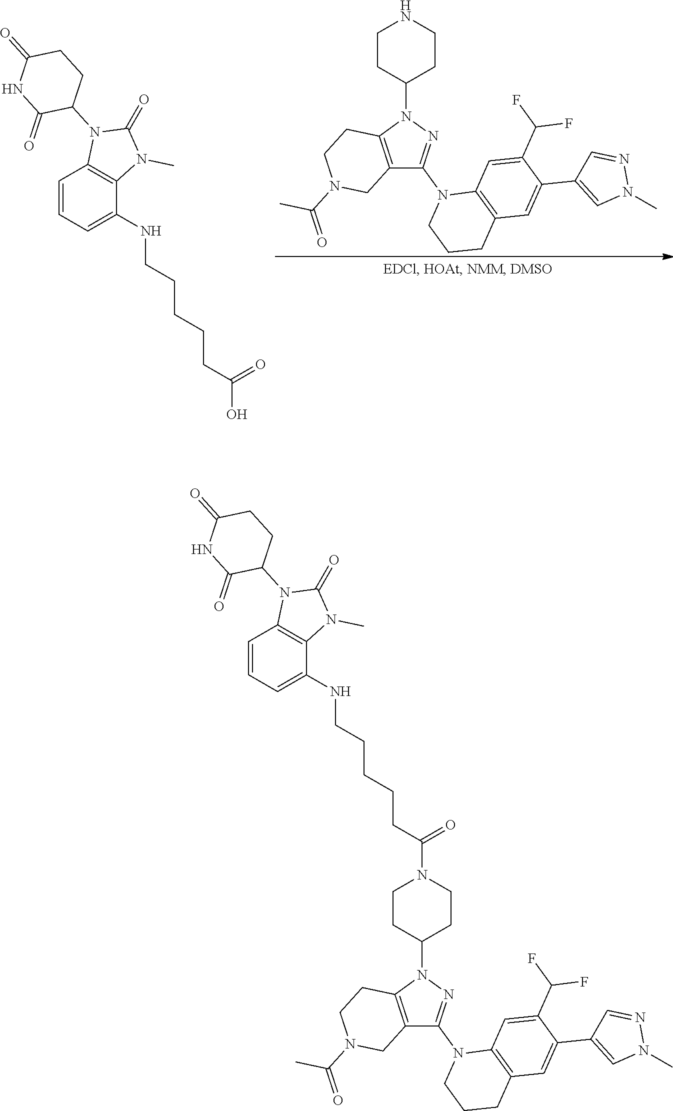 [SMILES: Cn1c(=O)n(C2CCC(=O)NC2=O)c2cccc(NCCCCCC(=O)O)c21 ; CC(=O)N1CCc2c(c(N3CCCc4cc(-c5cnn(C)c5)c(C(F)F)cc43)nn2C2CCNCC2)C1 ; CC(=O)N1CCc2c(c(N3CCCc4cc(-c5cnn(C)c5)c(C(F)F)cc43)nn2C2CCN(C(=O)CCCCCNc3cccc4c3n(C)c(=O)n4C3CCC(=O)NC3=O)CC2)C1]

P-110 was synthesized following the standard procedure for preparing P-001 (2.0 mg, yield 22%). MS (ESI) m/z: 881.2 [M+H]⁺.

### Example 196. 3-(4-((7-(4-(5-Acetyl-3-(7-(difluoromethyl)-6-(1-methyl-1H-pyrazol-4-yl)-3,4-dihydroquinolin-1(2H)-yl)-4,5,6,7-tetrahydro-1H-pyrazolo[4,3-c]pyridin-1-yl)piperidin-1-yl)-7-oxoheptyl)amino)-3-methyl-2-oxo-2,3-dihydro-1H-benzo[d]imidazol-1-yl)piperidine-2,6-dione (P-111)

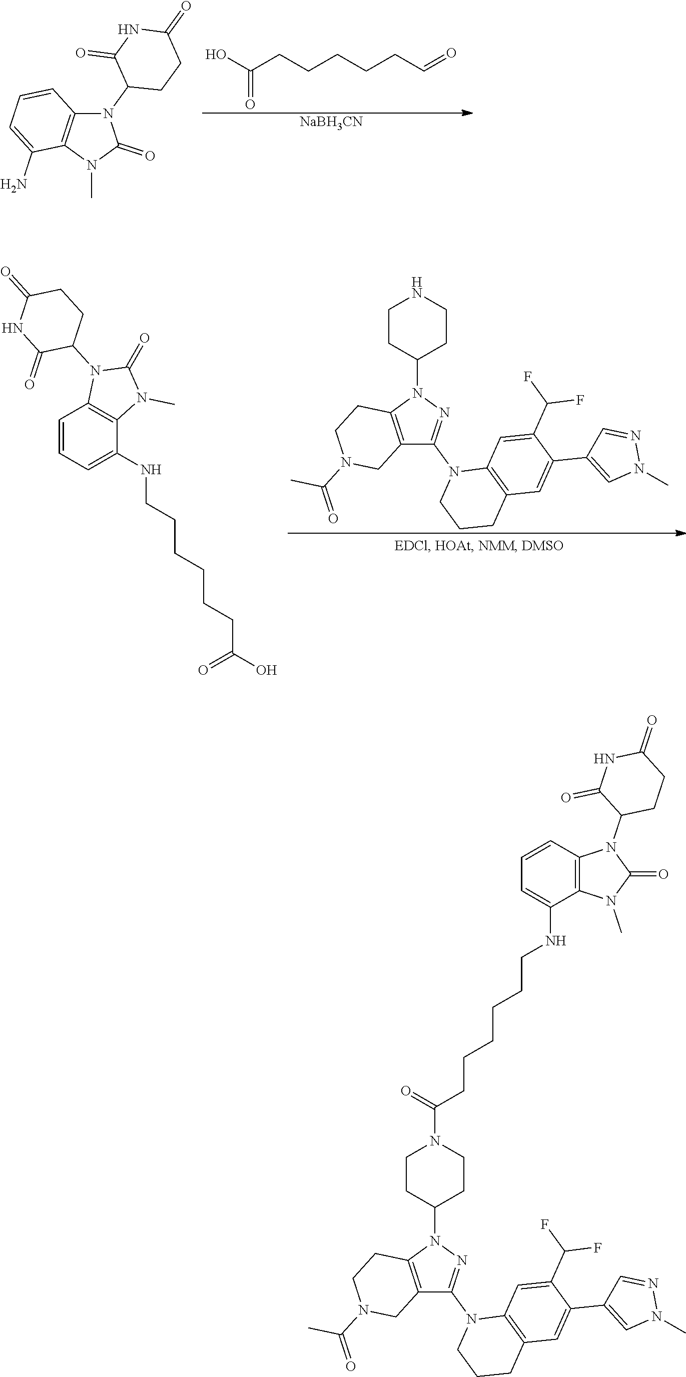

P-111 was synthesized following the similar procedure for preparing P-109 (2.5 mg, yield 11%). MS (ESI) m/z: 895.2 [M+H]⁺.

### Example 197. 4-((6-(4-(5-Acetyl-3-(7-(difluoromethyl)-6-(1-methyl-1H-pyrazol-4-yl)-3,4-dihydroquinolin-1(2H)-yl)-4,5,6,7-tetrahydro-1H-pyrazolo[4,3-c]pyridin-1-yl)piperidin-1-yl)hexyl)amino)-2-(2,6-dioxopiperidin-3-yl)isoindoline-1,3-dione (P-112)

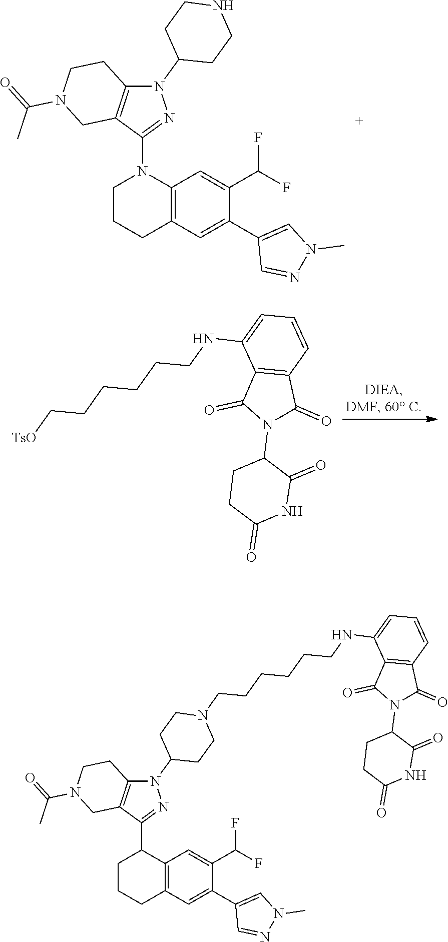

P-112 was synthesized following the similar procedure for preparing P-090 (45 mg, yield 55%). MS (ESI) m/z: 866.0 [M+H]⁺.

### Example 198. 4-((6-(4-(5-Acetyl-3-(7-(difluoromethyl)-6-(1-methyl-1H-pyrazol-4-yl)-3,4-dihydroquinolin-1(2H)-yl)-4,5,6,7-tetrahydro-1H-pyrazolo[4,3-c]pyridin-1-yl)piperidin-1-yl)hexyl)oxy)-2-(2,6-dioxopiperidin-3-yl)isoindoline-1,3-dione (P-113)

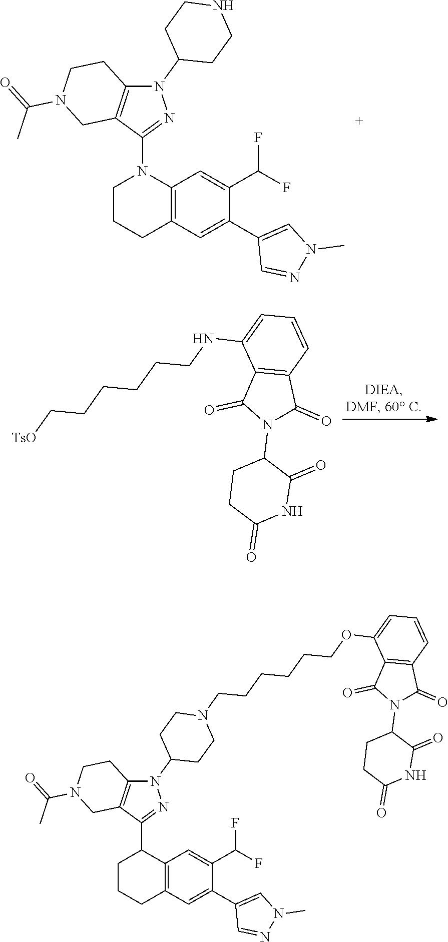

P-113 was synthesized following the similar procedure for preparing P-090 (55 mg, yield 67%). MS (ESI) m/z: 867.0 [M+H]⁺.

### Example 199. 3-(7-(Difluoromethyl)-6-(1-methyl-1H-pyrazol-4-yl)-3,4-dihydroquinolin-1(2H)-yl)-1-(1-(7-((1-(2,6-dioxopiperidin-3-yl)-3-methyl-2-oxo-2,3-dihydro-1H-benzo[d] imidazol-4-yl)amino)heptanoyl)piperidin-4-yl)-N-methyl-1,4,6,7-tetrahydro-5H-pyrazolo[4,3-c]pyridine-5-carboxamide (P-114)

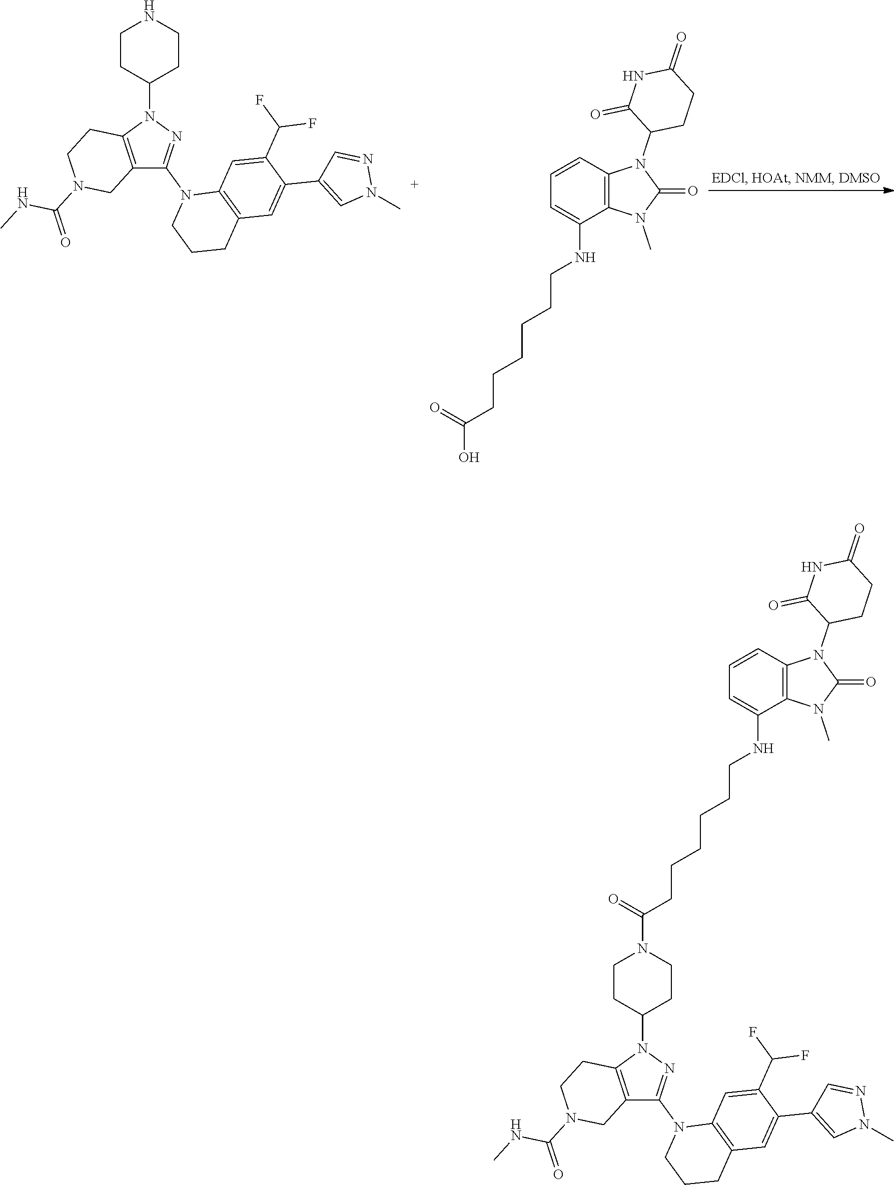

P-114 was synthesized following the standard procedure for preparing P-001 (1.4 mg, yield 6%). MS (ESI) m/z: 910.2 [M+H]⁺.

### Example 200. 3-(7-(Difluoromethyl)-6-(1-methyl-1H-pyrazol-4-yl)-3,4-dihydroquinolin-1(2H)-yl)-1-(1-(6-((1-(2,6-dioxopiperidin-3-yl)-3-methyl-2-oxo-2,3-dihydro-1H-benzo[d]imidazol-5-yl)amino)hexanoyl)piperidin-4-yl)-N-methyl-1,4,6,7-tetrahydro-5H-pyrazolo[4,3-c]pyridine-5-carboxamide (P-115)

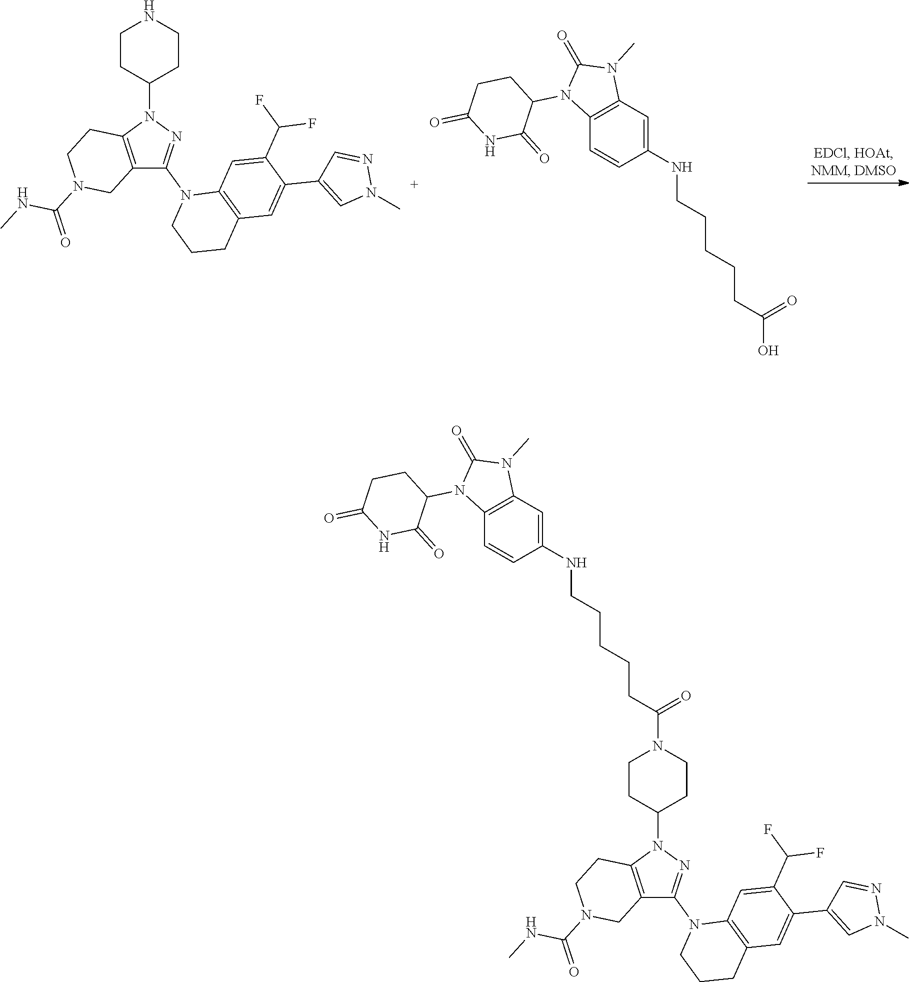

P-115 was synthesized following the standard procedure for preparing P-001 (4.4 mg, yield 21%). MS (ESI) m/z: 896.0 [M+H]⁺.

### Example 201. 3-(5-((6-(4-(5-Acetyl-3-(7-(difluoromethyl)-6-(1-methyl-1H-pyrazol-4-yl)-3,4-dihydroquinolin-1(2H)-yl)-4,5,6,7-tetrahydro-1H-pyrazolo[4,3-c]pyridin-1-yl)piperidin-1-yl)-6-oxohexyl)amino)-3-methyl-2-oxo-2,3-dihydro-1H-benzo[d]imidazol-1-yl)piperidine-2,6-dione (P-116)

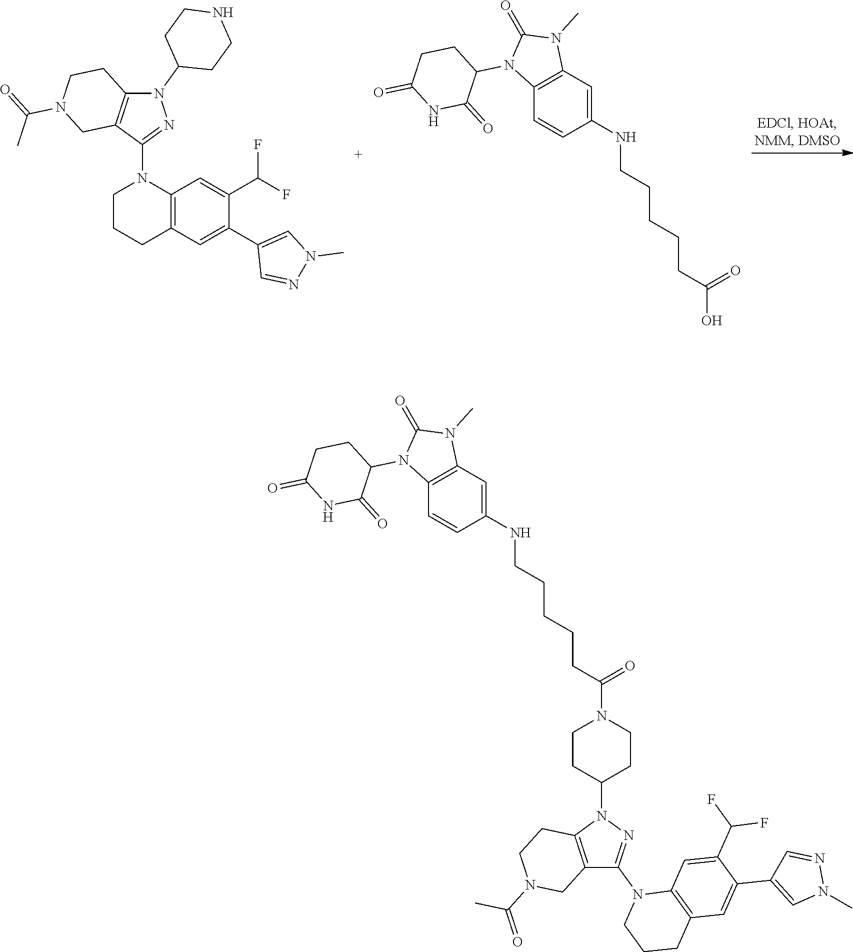 [SMILES: CC(=O)N1CCc2c(c(N3CCCc4cc(-c5cnn(C)c5)c(C(F)F)cc43)nn2C2CCNCC2)C1 ; Cn1c(=O)n(C2CCC(=O)NC2=O)c2ccc(NCCCCCC(=O)O)cc21 ; CC(=O)N1CCc2c(c(N3CCCc4cc(-c5cnn(C)c5)c(C(F)F)cc43)nn2C2CCN(C(=O)CCCCCNc3ccc4c(c3)n(C)c(=O)n4C3CCC(=O)NC3=O)CC2)C1]

P-116 was synthesized following the standard procedure for preparing P-001 (3.8 mg, yield 19%). MS (ESI) m/z: 881.0 [M+H]⁺.

### Example 202. 4-((6-(4-(5-Acetyl-3-(7-(difluoromethyl)-6-(1-methyl-1H-pyrazol-4-yl)-3,4-dihydroquinolin-1(2H)-yl)-4,5,6,7-tetrahydro-1H-pyrazolo[4,3-c]pyridin-1-yl)piperidin-1-yl)-6-oxohexyl)oxy)-2-(2,6-dioxopiperidin-3-yl)isoindoline-1,3-dione (P-117)

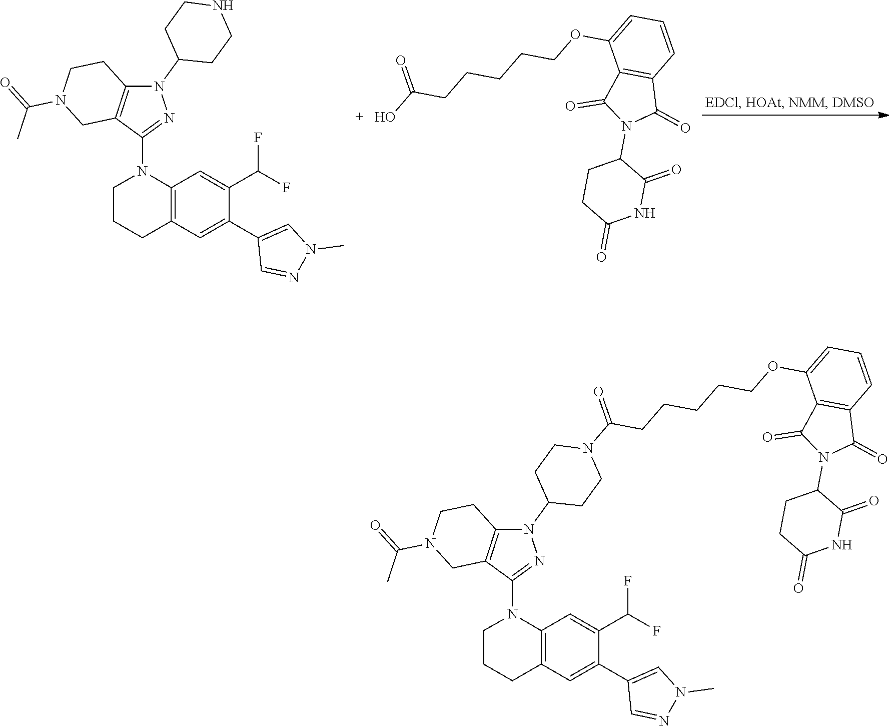 [SMILES: CC(=O)N1CCc2c(c(N3CCCc4cc(-c5cnn(C)c5)c(C(F)F)cc43)nn2C2CCNCC2)C1 ; O=C(O)CCCCCOc1cccc2c1C(=O)N(C1CCC(=O)NC1=O)C2=O ; CC(=O)N1CCc2c(c(N3CCCc4cc(-c5cnn(C)c5)c(C(F)F)cc43)nn2C2CCN(C(=O)CCCCCOc3cccc4c3C(=O)N(C3CCC(=O)NC3=O)C4=O)CC2)C1]

P-117 was synthesized following the standard procedure for preparing P-001 (37.1 mg, yield 72%). MS (ESI) m/z: 880.9 [M+H]⁺.

### Example 203. 4-(7-(4-(5-Acetyl-3-(7-(difluoromethyl)-6-(1-methyl-1H-pyrazol-4-yl)-3,4-dihydroquinolin-1(2H)-yl)-4,5,6,7-tetrahydro-1H-pyrazolo[4,3-c]pyridin-1-yl)piperidin-1-yl)-7-oxoheptyl)-2-(2,6-dioxopiperidin-3-yl)isoindoline-1,3-dione (P-118)

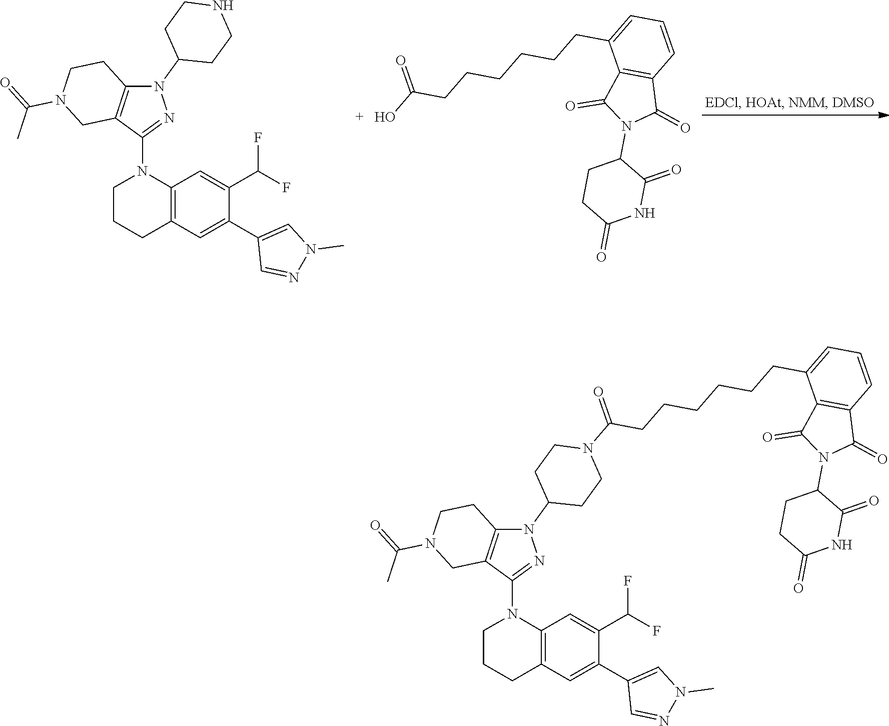 [SMILES: CC(=O)N1CCc2c(c(N3CCCc4cc(-c5cnn(C)c5)c(C(F)F)cc43)nn2C2CCNCC2)C1 ; O=C(O)CCCCCCc1cccc2c1C(=O)N(C1CCC(=O)NC1=O)C2=O ; CC(=O)N1CCc2c(c(N3CCCc4cc(-c5cnn(C)c5)c(C(F)F)cc43)nn2C2CCN(C(=O)CCCCCCc3cccc4c3C(=O)N(C3CCC(=O)NC3=O)C4=O)CC2)C1]

P-118 was synthesized following the standard procedure for preparing P-001 (37.0 mg, yield 72%). MS (ESI) m/z: 879.0 [M+H]⁺.

### Example 204. 5-((8-(4-(5-Acetyl-3-(7-(difluoromethyl)-6-(1-methyl-1H-pyrazol-4-yl)-3,4-dihydroquinolin-1(2H)-yl)-4,5,6,7-tetrahydro-1H-pyrazolo[4,3-c]pyridin-1-yl)piperidin-1-yl)-8-oxooctyl)amino)-2-(2,6-dioxopiperidin-3-yl)isoindoline-1,3-dione (P-119)

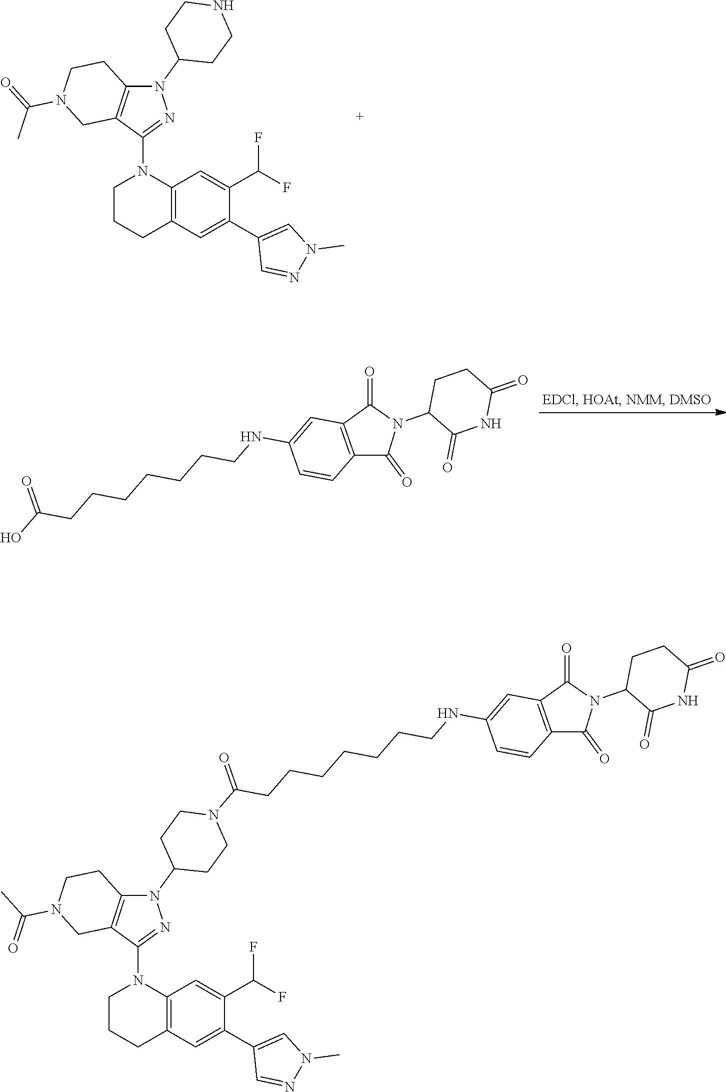 [SMILES: CC(=O)N1CCc2c(c(N3CCCc4cc(-c5cnn(C)c5)c(C(F)F)cc43)nn2C2CCNCC2)C1 ; O=C(O)CCCCCCCNc1ccc2c(c1)C(=O)N(C1CCC(=O)NC1=O)C2=O ; CC(=O)N1CCc2c(c(N3CCCc4cc(-c5cnn(C)c5)c(C(F)F)cc43)nn2C2CCN(C(=O)CCCCCCCNc3ccc4c(c3)C(=O)N(C3CCC(=O)NC3=O)C4=O)CC2)C1]

P-119 was synthesized following the standard procedure for preparing P-001 (28.5 mg, yield 80%). MS (ESI) m/z: 908.0 [M+H]⁺.

### Example 205. 5-((6-(4-(5-Acetyl-3-(7-(difluoromethyl)-6-(1-methyl-1H-pyrazol-4-yl)-3,4-dihydroquinolin-1(2H)-yl)-4,5,6,7-tetrahydro-1H-pyrazolo[4,3-c]pyridin-1-yl)piperidin-1-yl)-6-oxohexyl)amino)-2-(2,6-dioxopiperidin-3-yl)isoindoline-1,3-dione (P-120)

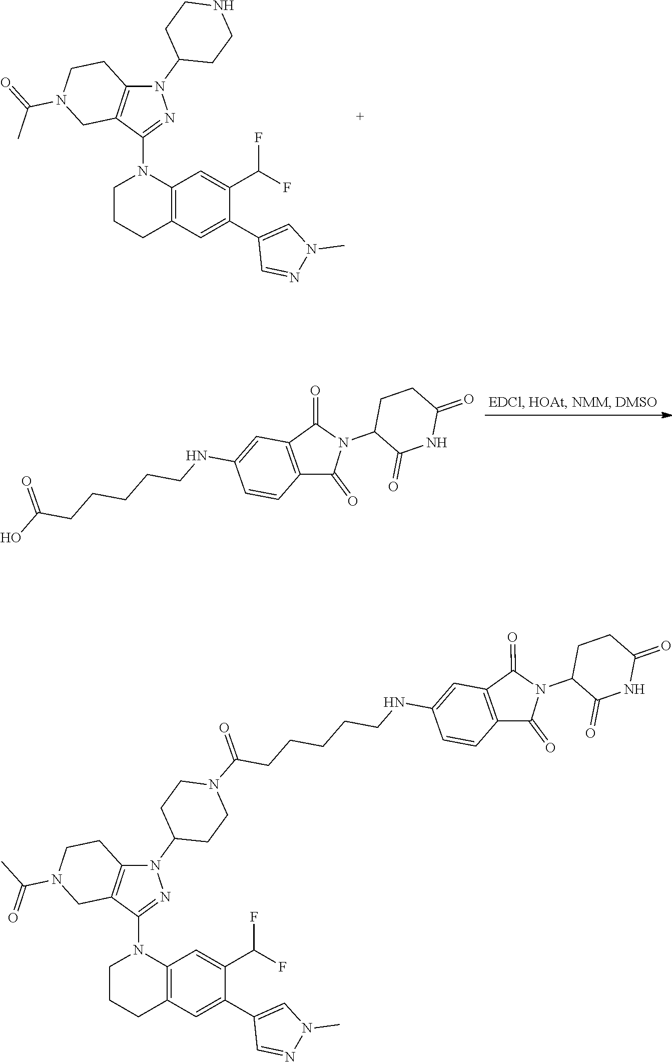 [SMILES: CC(=O)N1CCc2c(c(N3CCCc4cc(-c5cnn(C)c5)c(C(F)F)cc43)nn2C2CCNCC2)C1 ; O=C(O)CCCCCNc1ccc2c(c1)C(=O)N(C1CCC(=O)NC1=O)C2=O ; CC(=O)N1CCc2c(c(N3CCCc4cc(-c5cnn(C)c5)c(C(F)F)cc43)nn2C2CCN(C(=O)CCCCCNc3ccc4c(c3)C(=O)N(C3CCC(=O)NC3=O)C4=O)CC2)C1]

P-120 was synthesized following the standard procedure for preparing P-001 (36.8 mg, yield 71%). MS (ESI) m/z: 879.9 [M+H]⁺.

### Example 206. 3-(4-((6-(4-(5-Acetyl-3-(7-(difluoromethyl)-6-(1-methyl-1H-pyrazol-4-yl)-3,4-dihydroquinolin-1(2H)-yl)-4,5,6,7-tetrahydro-1H-pyrazolo[4,3-c]pyridin-1-yl)piperidin-1-yl)-6-oxohexyl)oxy)-1-oxoisoindolin-2-yl)piperidine-2,6-dione (P-121)

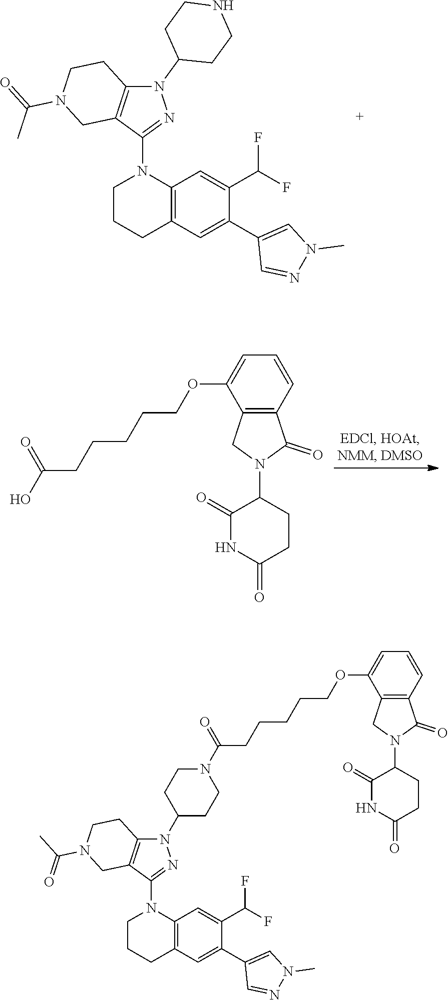

P-121 was synthesized following the standard procedure for preparing P-001 (42 mg, yield 76%). MS (ESI) m/z: 867.0 [M+H]⁺.

### Example 207. 3-(4-(7-(4-(5-Acetyl-3-(7-(difluoromethyl)-6-(1-methyl-1H-pyrazol-4-yl)-3,4-dihydroquinolin-1(2H)-yl)-4,5,6,7-tetrahydro-1H-pyrazolo[4,3-c]pyridin-1-yl)piperidin-1-yl)-7-oxohept-1-yn-1-yl)-1-oxoisoindolin-2-yl)piperidine-2,6-dione (P-122)

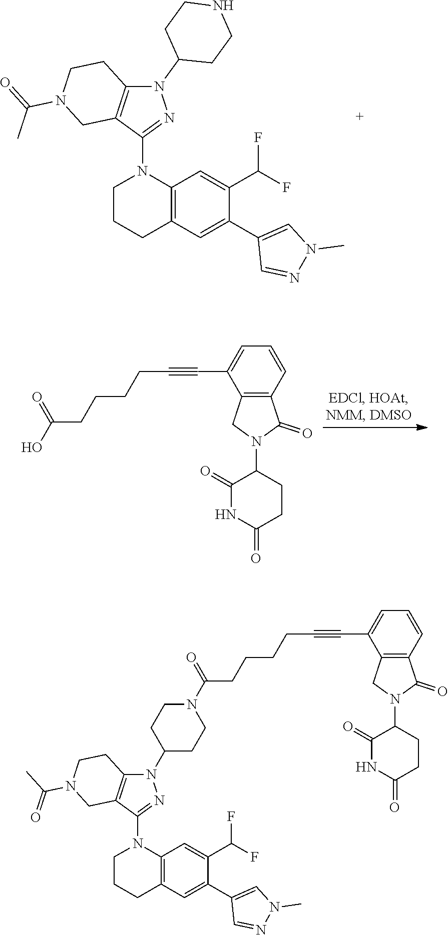

P-122 was synthesized following the standard procedure for preparing P-001 (38.8 mg, yield 77%). MS (ESI) m/z: 860.9 [M+H]⁺.

### Example 208. 4-(((1-(2-(4-(5-Acetyl-3-(7-(difluoromethyl)-6-(1-methyl-1H-pyrazol-4-yl)-3,4-dihydroquinolin-1(2H)-yl)-4,5,6,7-tetrahydro-1H-pyrazolo[4,3-c]pyridin-1-yl)piperidin-1-yl)-2-oxoethyl)piperidin-4-yl)methyl)amino)-2-(2,6-dioxopiperidin-3-yl)isoindoline-1,3-dione (P-123)

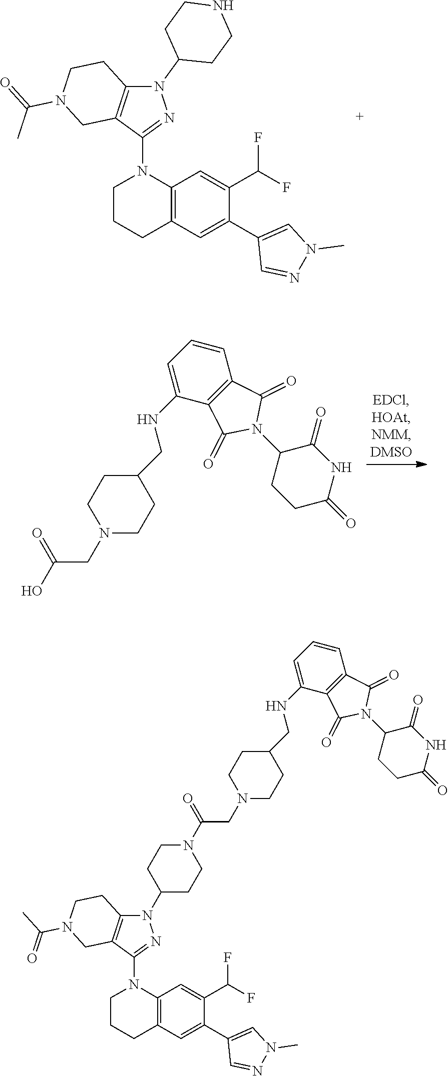

P-123 was synthesized following the standard procedure for preparing P-001 (36.9 mg, yield 68%). MS (ESI) m/z: 921.0 [M+H]⁺.

### Example 209. N-(3-(4-(5-Acetyl-3-(7-(difluoromethyl)-6-(1-methyl-1H-pyrazol-4-yl)-3,4-dihydroquinolin-1(2H)-yl)-4,5,6,7-tetrahydro-1H-pyrazolo[4,3-c]pyridin-1-yl)piperidin-1-yl)propyl)-2-((2-(2,6-dioxopiperidin-3-yl)-1,3-dioxoisoindolin-4-yl)amino)acetamide (P-124)

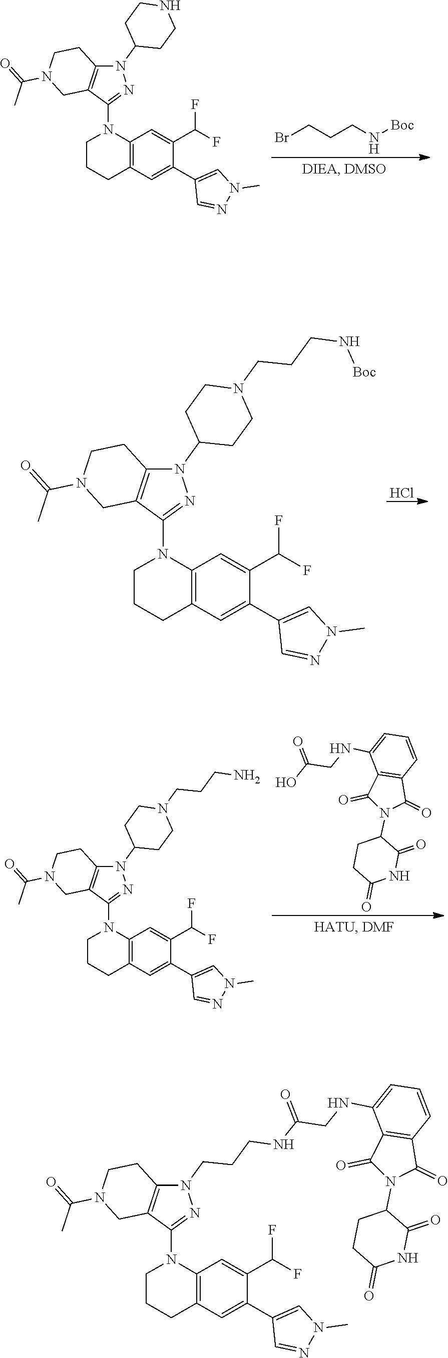

### Step 1: Synthesis of tert-butyl (3-(4-(5-acetyl-3-(7-(difluoromethyl)-6-(1-methyl-1H-pyrazol-4-yl)-3,4-dihydroquinolin-1(2H)-yl)-4,5,6,7-tetrahydro-1H-pyrazolo[4,3-c]pyridin-1-yl)piperidin-1-yl)propyl)carbamate

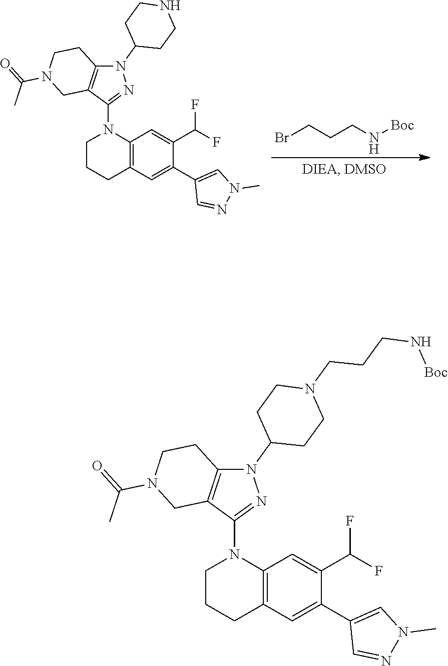 [SMILES: CC(=O)N1CCc2c(c(N3CCCc4cc(-c5cnn(C)c5)c(C(F)F)cc43)nn2C2CCNCC2)C1 ; CC(C)(C)OC(=O)NCCCBr ; CC(=O)N1CCc2c(c(N3CCCc4cc(-c5cnn(C)c5)c(C(F)F)cc43)nn2C2CCN(CCCNC(=O)OC(C)(C)C)CC2)C1]

A mixture of 1-(3-(7-(difluoromethyl)-6-(1-methyl-1H-pyrazol-4-yl)-3,4-dihydroquinolin-1(2H)-yl)-1-(piperidin-4-yl)-1,4,6,7-tetrahydro-5H-pyrazolo[4,3-c]pyridin-5-yl)ethan-1-one (5 mg, 0.0098 mmol), tert-butyl (3-bromopropyl)carbamate (3.04 mg, 0.0128 mmol), NaI (1.47 mg, 0.0098 mmol), and DIEA (3.80 mg, 0.029 mmol) in DMF (2 mL) was stirred at 60° C. for 16 h. After the reaction mixture was concentrated, the resulting residue was purified by prep-TLC to give the desired product (6 mg, 92% yield). MS (ESI) m/z: 668.1 [M+H]⁺.

### Step 2: Synthesis of 1-(1-(1-(3-aminopropyl)piperidin-4-yl)-3-(7-(difluoromethyl)-6-(1-methyl-1H-pyrazol-4-yl)-3,4-dihydroquinolin-1(2H)-yl)-1,4,6,7-tetrahydro-5H-pyrazolo[4,3-c]pyridin-5-yl)ethan-1-one

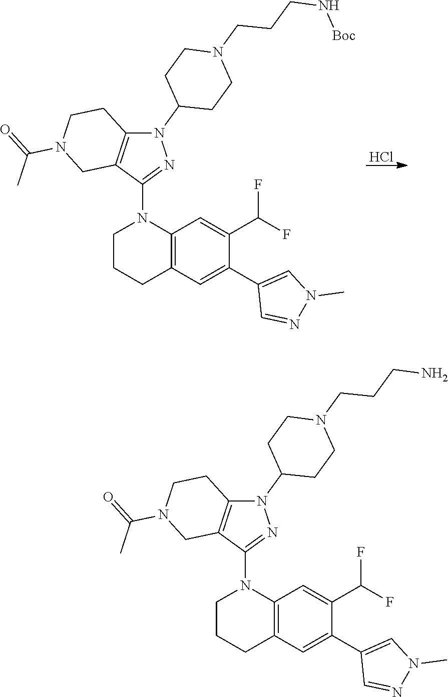 [SMILES: CC(=O)N1CCc2c(c(N3CCCc4cc(-c5cnn(C)c5)c(C(F)F)cc43)nn2C2CCN(CCCNC(=O)OC(C)(C)C)CC2)C1 ; Cl ; CC(=O)N1CCc2c(c(N3CCCc4cc(-c5cnn(C)c5)c(C(F)F)cc43)nn2C2CCN(CCCN)CC2)C1]

To a solution of tert-butyl (3-(4-(5-acetyl-3-(7-(difluoromethyl)-6-(1-methyl-1H-pyrazol-4-yl)-3,4-dihydroquinolin-1(2H)-yl)-4,5,6,7-tetrahydro-1H-pyrazolo[4,3-c]pyridin-1-yl)piperidin-1-yl)propyl)carbamate (13 mg, 0.0195 mmol) in DCM (4 mL) was added HCl/dioxane (4 mL). The resulting mixture was stirred at room temperature for 2 h. After the reaction mixture was concentrated, the resulting residue was washed with EtOAc to give the desired product (10 mg, 91% yield). MS (ESI) m/z: 567.8 [M+H]⁺.

### Step 3: Synthesis of N-(3-(4-(5-acetyl-3-(7-(difluoromethyl)-6-(1-methyl-1H-pyrazol-4-yl)-3,4-dihydroquinolin-1(2H)-yl)-4,5,6,7-tetrahydro-1H-pyrazolo[4,3-c]pyridin-1-yl)piperidin-1-yl)propyl)-2-((2-(2,6-dioxopiperidin-3-yl)-1,3-dioxoisoindolin-4-yl)amino)acetamide (P-124)

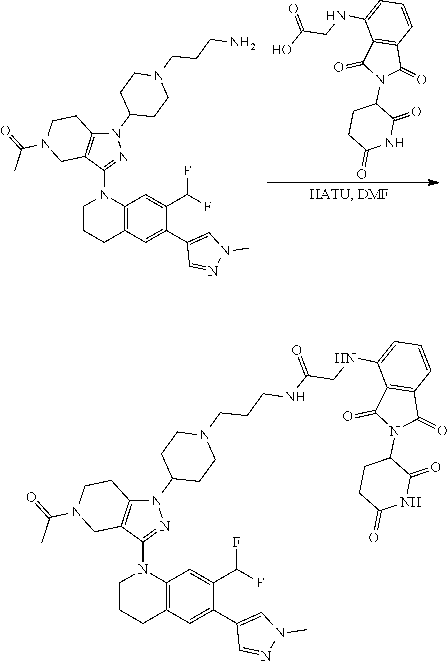 [SMILES: CC(=O)N1CCc2c(c(N3CCCc4cc(-c5cnn(C)c5)c(C(F)F)cc43)nn2C2CCN(CCCN)CC2)C1 ; O=C(O)CNc1cccc2c1C(=O)N(C1CCC(=O)NC1=O)C2=O ; CC(=O)N1CCc2c(c(N3CCCc4cc(-c5cnn(C)c5)c(C(F)F)cc43)nn2C2CCN(CCCNC(=O)CNc3cccc4c3C(=O)N(C3CCC(=O)NC3=O)C4=O)CC2)C1]

P-124 was synthesized following the standard procedure for preparing P-001 (16 mg, yield 99%). MS (ESI) m/z: 881.0 [M+H]⁺.

### Example 210. 3-(7-(Difluoromethyl)-6-(1-methyl-1H-pyrazol-4-yl)-3,4-dihydroquinolin-1(2H)-yl)-1-(1-(5-((1-(2,6-dioxopiperidin-3-yl)-3-methyl-2-oxo-2,3-dihydro-1H-benzo[d]imidazol-5-yl)amino)pentanoyl)piperidin-4-yl)-N-methyl-1,4,6,7-tetrahydro-5H-pyrazolo[4,3-c]pyridine-5-carboxamide (P-125)

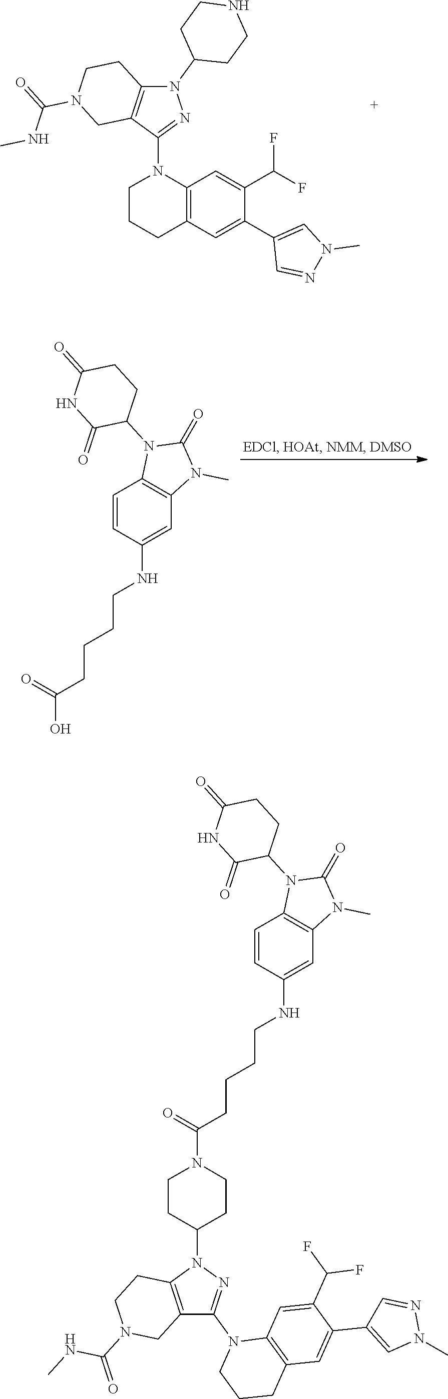

P-125 was synthesized following the standard procedure for preparing P-001 (1.46 mg, yield 9%). MS (ESI) ink: 882.0 [M+H]⁺.

### Example 211. 3-(5-((5-(4-(5-Acetyl-3-(7-(difluoromethyl)-6-(1-methyl-1H-pyrazol-4-yl)-3,4-dihydroquinolin-1(2H)-yl)-4,5,6,7-tetrahydro-1H-pyrazolo[4,3-c]pyridin-1-yl)piperidin-1-yl)-5-oxopentyl)amino)-3-methyl-2-oxo-2,3-dihydro-1H-benzo[d]imidazol-1-yl)piperidine-2,6-dione (P-126)

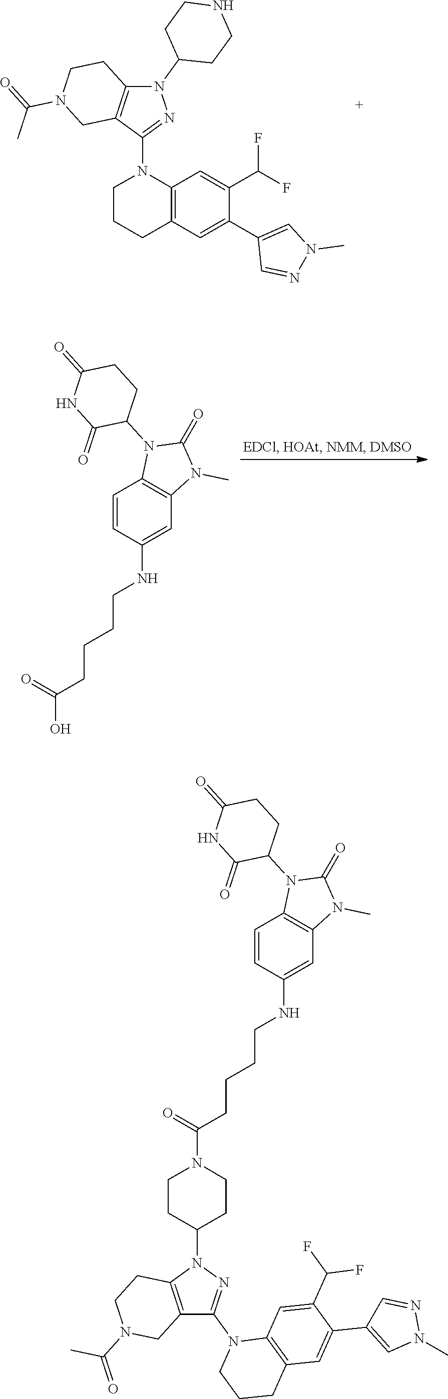

P-126 was synthesized following the standard procedure for preparing P-001 (1.75 mg, yield 10%). MS (ESI) ink: 866.9 [M+H]⁺.

### Example 212. 3-(7-(Difluoromethyl)-6-(1-methyl-1H-pyrazol-4-yl)-3,4-dihydroquinolin-1(2H)-yl)-1-(1-(5-((1-(2,6-dioxopiperidin-3-yl)-3-methyl-2-oxo-2,3-dihydro-1H-benzo[d]imidazol-4-yl)amino)pentanoyl)piperidin-4-yl)-N-methyl-1,4,6,7-tetrahydro-5H-pyrazolo[4,3-c]pyridine-5-carboxamide (P-127)

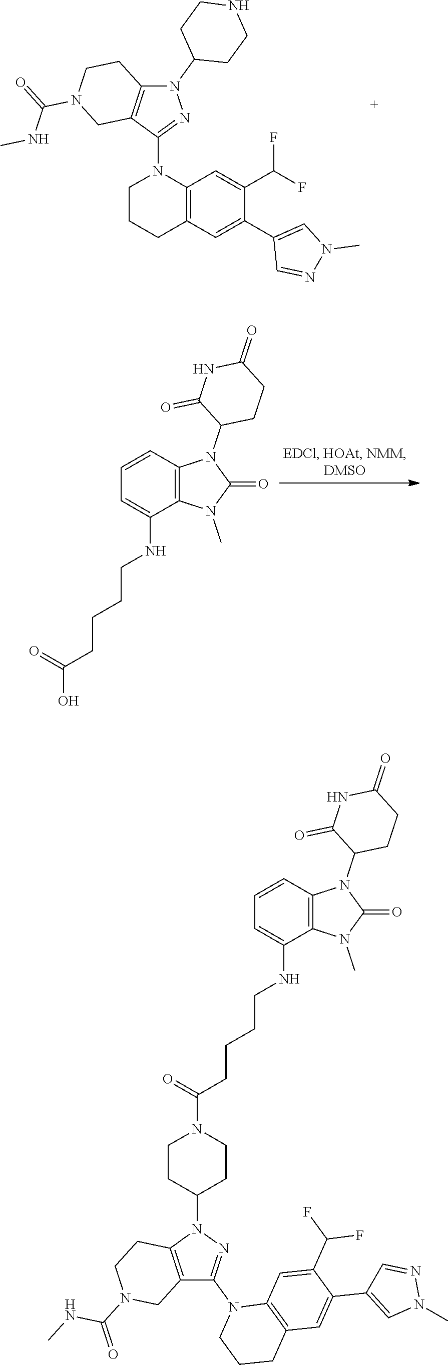

P-127 was synthesized following the standard procedure for preparing P-001 (2.1 mg, yield 13%). MS (ESI) m/z: 882.0 [M+H]⁺.

### Example 213. 3-(4-((5-(4-(5-Acetyl-3-(7-(difluoromethyl)-6-(1-methyl-1H-pyrazol-4-yl)-3,4-dihydroquinolin-1(2H)-yl)-4,5,6,7-tetrahydro-1H-pyrazolo[4,3-c]pyridin-1-yl)piperidin-1-yl)-5-oxopentyl)amino)-3-methyl-2-oxo-2,3-dihydro-1H-benzo[d]imidazol-1-yl)piperidine-2,6-dione (P-128)

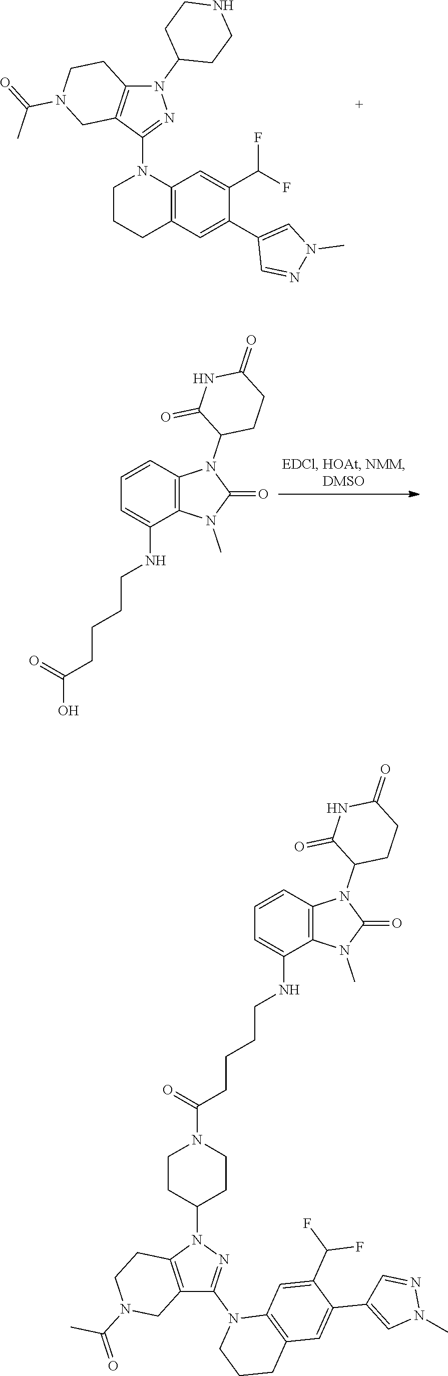

P-128 was synthesized following the standard procedure for preparing P-001 (3.2 mg, yield 19%). MS (ESI) m/z: 867.0 [M+H]⁺.

### Example 214. 3-(7-(Difluoromethyl)-6-(1-methyl-1H-pyrazol-4-yl)-3,4-dihydroquinolin-1(2H)-yl)-1-(1-(7-((1-(2,6-dioxopiperidin-3-yl)-3-methyl-2-oxo-2,3-dihydro-1H-benzo[d]imidazol-5-yl)amino)heptanoyl)piperidin-4-yl)-N-methyl-1,4,6,7-tetrahydro-5H-pyrazolo[4,3-c]pyridine-5-carboxamide (P-129)

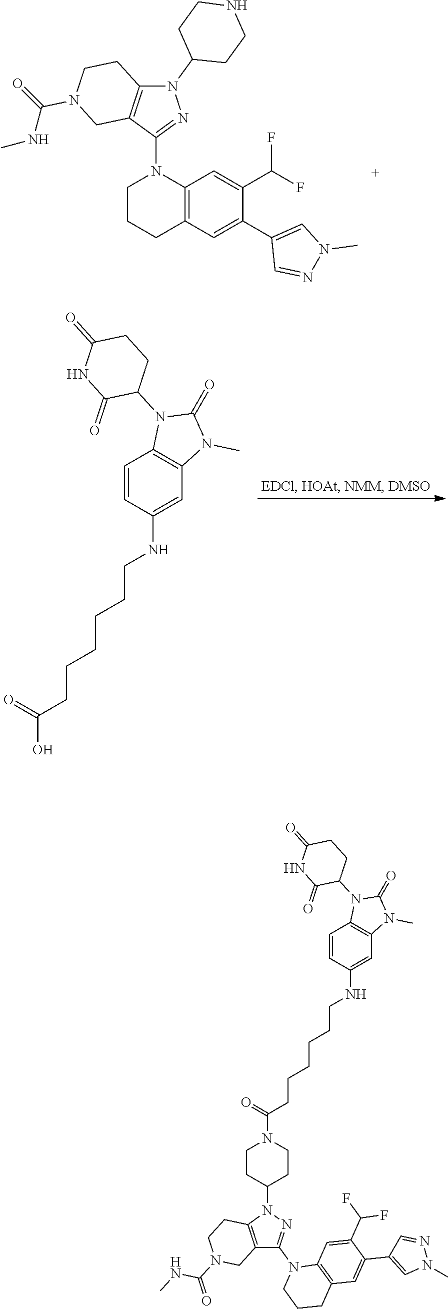

P-129 was synthesized following the standard procedure for preparing P-001 (4.2 mg, yield 48%). MS (ESI) m/z: 910.1 [M+H]⁺.

### Example 215. 3-(5-((7-(4-(5-Acetyl-3-(7-(difluoromethyl)-6-(1-methyl-1H-pyrazol-4-yl)-3,4-dihydroquinolin-1(2H)-yl)-4,5,6,7-tetrahydro-1H-pyrazolo[4,3-c]pyridin-1-yl)piperidin-1-yl)-7-oxoheptyl)amino)-3-methyl-2-oxo-2,3-dihydro-1H-benzo[d]imidazol-1-yl)piperidine-2,6-dione (P-130)

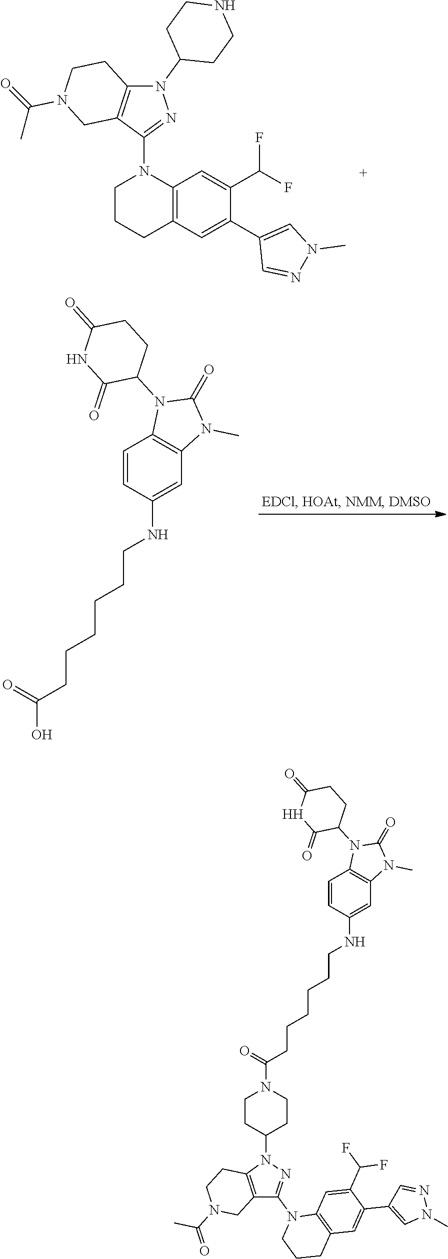

P-130 was synthesized following the standard procedure for preparing P-001 (3.6 mg, yield 41%). MS (ESI) m/z: 895.1 [M+H]⁺.

### Example 216. 4-(2-(1-(2-(4-(5-Acetyl-3-(7-(difluoromethyl)-6-(1-methyl-1H-pyrazol-4-yl)-3,4-dihydroquinolin-1(2H)-yl)-4,5,6,7-tetrahydro-1H-pyrazolo[4,3-c]pyridin-1-yl)piperidin-1-yl)-2-oxoethyl)piperidin-4-yl)ethyl)-2-(2,6-dioxopiperidin-3-yl)isoindoline-1,3-dione (P-131)

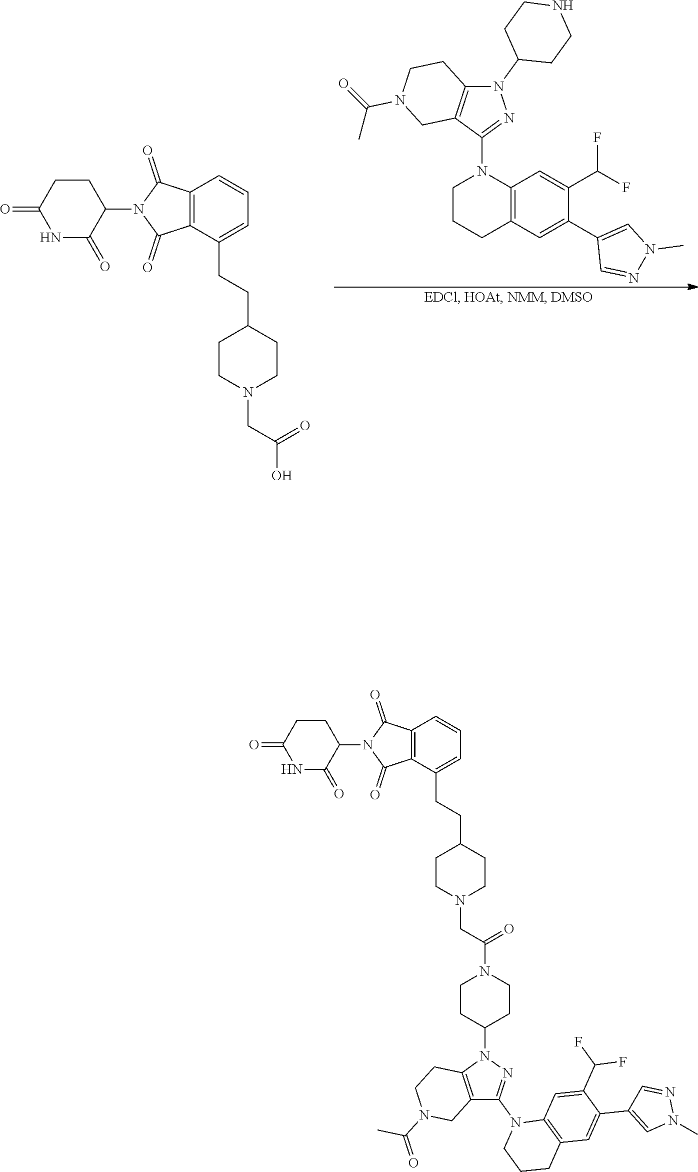

P-131 was synthesized following the standard procedure for preparing P-104 (107 mg, yield 25%). MS (ESI) m/z: 920.1 [M+H]⁺.

### Example 217. 4-(7-(4-(5-Acetyl-3-(7-(difluoromethyl)-6-(1-methyl-1H-pyrazol-4-yl)-3,4-dihydroquinolin-1(2H)-yl)-4,5,6,7-tetrahydro-1H-pyrazolo[4,3-c]pyridin-1-yl)piperidin-1-yl)heptyl)-2-(2,6-dioxopiperidin-3-yl)isoindoline-1,3-dione (P-132)

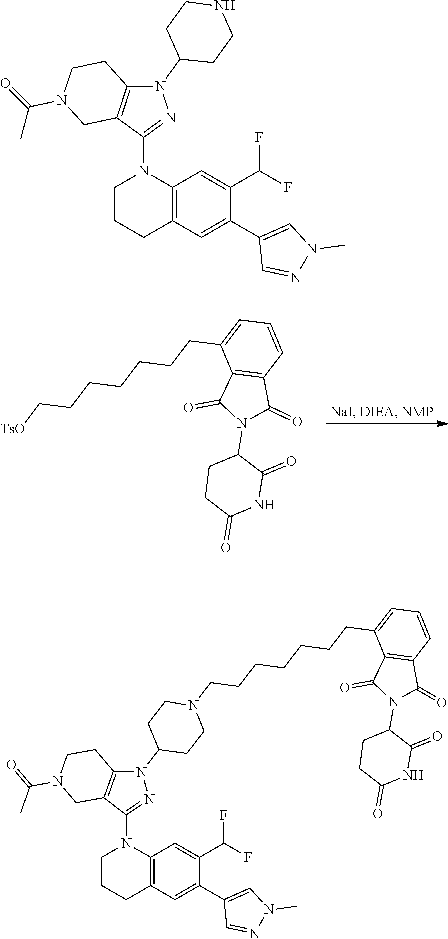

P-132 was synthesized following the standard procedure for preparing P-097 (22 mg, yield 38%). MS (ESI) m/z: 865.1 [M+H]⁺.

### Example 218. 2-(4-(1-(5-Acetyl-1-(tetrahydro-2H-pyran-4-yl)-4,5,6,7-tetrahydro-1H-pyrazolo[4,3-c]pyridin-3-yl)-7-(difluoromethyl)-1,2,3,4-tetrahydroquinolin-6-yl)-1H-pyrazol-1-yl)-N-(7-((2-(2,6-dioxopiperidin-3-yl)-1,3-dioxoisoindolin-5-yl)amino)heptyl)acetamide (P-133)

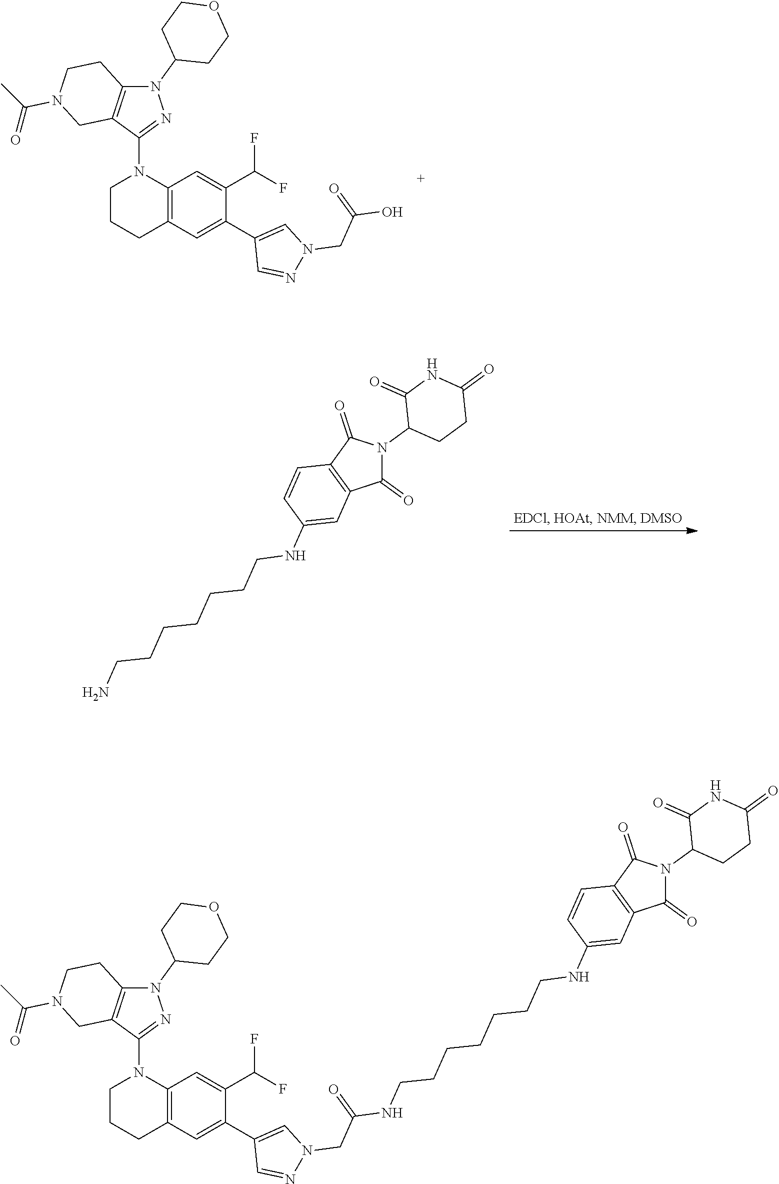

P-133 was synthesized following the standard procedure for preparing P-026 (10.5 mg, yield 23%). MS (ESI) m/z: 924.0 [M+H]⁺.

### Example 219. 4-(2-(4-(5-Acetyl-3-(7-(difluoromethyl)-6-(1-methyl-1H-pyrazol-4-yl)-3,4-dihydroquinolin-1(2H)-yl)-4,5,6,7-tetrahydro-1H-pyrazolo[4,3-c]pyridin-1-yl)-[1,4′-bipiperidin]-1′-yl)ethoxy)-2-(2,6-dioxopiperidin-3-yl)isoindoline-1,3-dione (P-134)

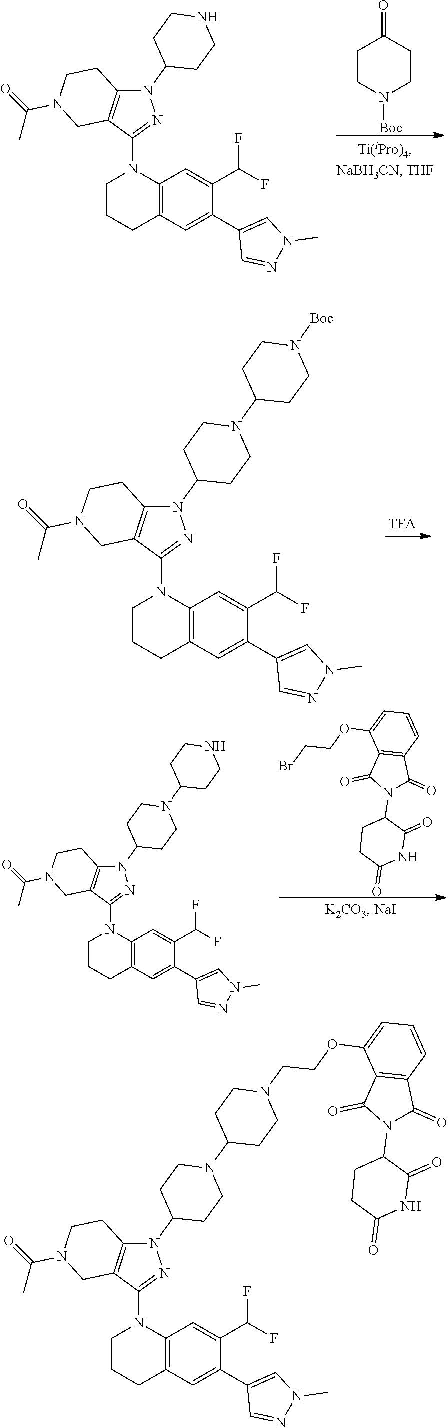

### Step 1: Synthesis of tert-butyl 4-(5-acetyl-3-(7-(difluoromethyl)-6-(1-methyl-1H-pyrazol-4-yl)-3,4-dihydroquinolin-1(2H)-yl)-4,5,6,7-tetrahydro-1H-pyrazolo[4,3-c]pyridin-1-yl)-[1,4′-bipiperidine]-1′-carboxylate

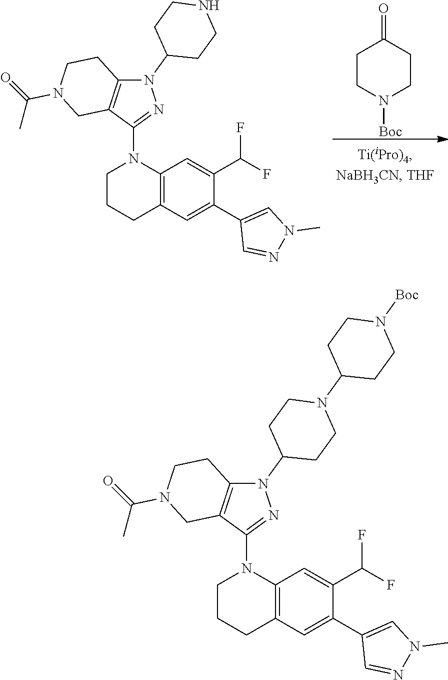

A mixture of 1-(3-(7-(difluoromethyl)-6-(1-methyl-1H-pyrazol-4-yl)-3,4-dihydroquinolin-1(2H)-yl)-1-(piperidin-4-yl)-1,4,6,7-tetrahydro-5H-pyrazolo[4,3-c]pyridin-5-yl)ethan-1-one (150 mg, 0.295 mmol), tert-butyl 4-oxopiperidine-1-carboxylate (88 mg, 0.442 mmol), and Ti(iPrO)₄ (168 mg, 0.59 mmol) in anhydrous THF (5 mL) was refluxed for 2.5 h under N₂. After the reaction was cooled to room temperature, NaBH₃CN (74 mg, 1.18 mmol) was added. After the resulting mixture was stirred at room temperature overnight, the reaction mixture was purified by reverse phase chromatography to give the desired product (88 mg, 43% yield) as a white solid. MS (ESI) m/z: 694.1 [M+H]⁺.

### Step 2: Synthesis of 1-(1-([1,4′-bipiperidin]-4-yl)-3-(7-(difluoromethyl)-6-(1-methyl-1H-pyrazol-4-yl)-3,4-dihydroquinolin-1(2H)-yl)-1,4,6,7-tetrahydro-5H-pyrazolo[4,3-c]pyridin-5-yl)ethan-1-one

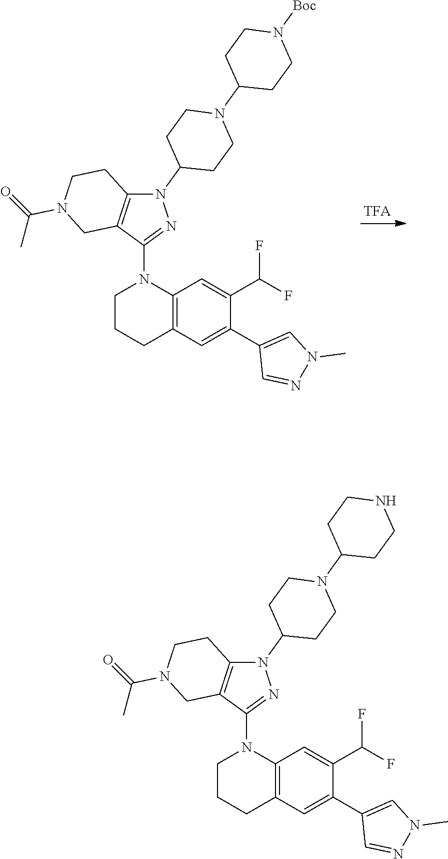

A mixture of tert-butyl 4-(5-acetyl-3-(7-(difluoromethyl)-6-(1-methyl-1H-pyrazol-4-yl)-3,4-dihydroquinolin-1(2H)-yl)-4,5,6,7-tetrahydro-1H-pyrazolo[4,3-c]pyridin-1-yl)-[1,4′-bipiperidine]-1′-carboxylate (88 mg, 0.127 mmol) in TFA/DCM (2 mL, 1:1) was stirred at room temperature for 1 h. The reaction was purified by reverse phase chromatography to give the desire product (78 mg, 99% yield) as a white solid. MS (ESI) m/z: 593.8 [M+H]⁺.

### Step 3: Synthesis of 1-(1-([1,4′-bipiperidin]-4-yl)-3-(7-(difluoromethyl)-6-(1-methyl-1H-pyrazol-4-yl)-3,4-dihydroquinolin-1(2H)-yl)-1,4,6,7-tetrahydro-5H-pyrazolo[4,3-c]pyridin-5-yl)ethan-1-one (P-134)

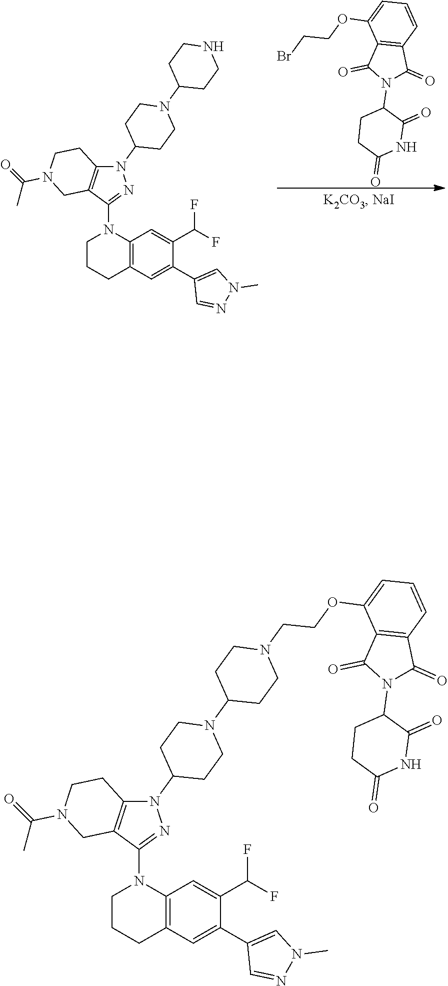

P-134 was synthesized following the standard procedure for preparing P-090 (13.2 mg, yield 38%). MS (ESI) m/z: 894.0 [M+H]⁺.

### Example 220. 3-(3-(8-(4-(5-Acetyl-3-(7-(difluoromethyl)-6-(1-methyl-1H-pyrazol-4-yl)-3,4-dihydroquinolin-1(2H)-yl)-4,5,6,7-tetrahydro-1H-pyrazolo[4,3-c]pyridin-1-yl)piperidin-1-yl)-8-oxooctyl)-2-oxo-2,3-dihydro-1H-benzo[d]imidazol-1-yl)piperidine-2,6-dione (P-135)

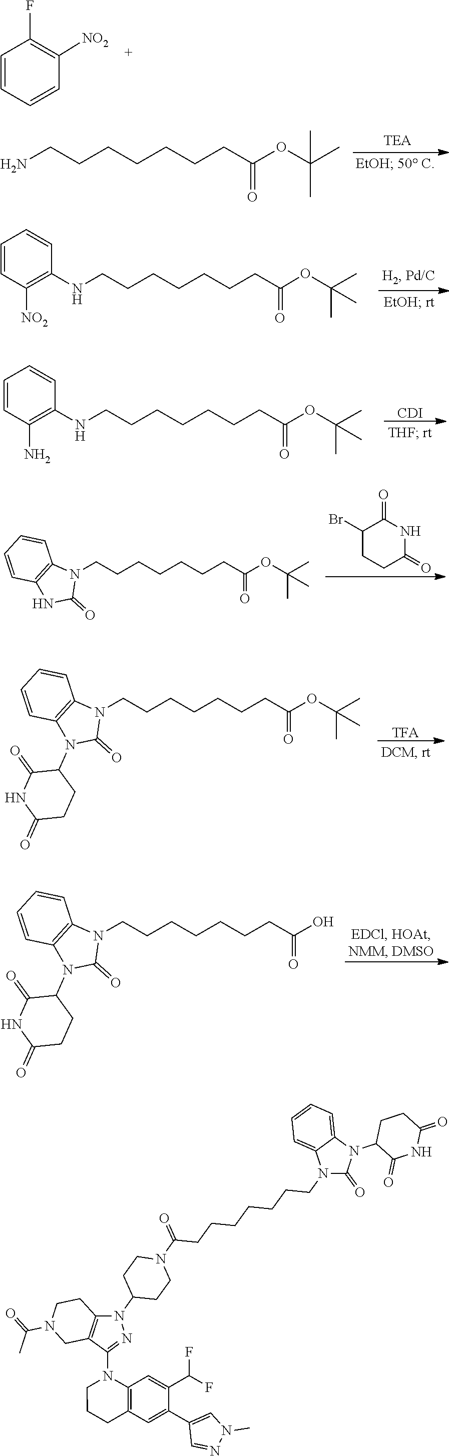

P-135 was synthesized following the standard procedure for preparing P-105 (25 mg, yield 37%). MS (ESI) m/z: 880.1 [M+H]⁺.

### Example 221. 6-(4-(5-Acetyl-3-(7-(difluoromethyl)-6-(1-methyl-1H-pyrazol-4-yl)-3,4-dihydroquinolin-1(2H)-yl)-4,5,6,7-tetrahydro-1H-pyrazolo[4,3-c]pyridin-1-yl)piperidin-1-yl)-N-(2-(2,6-dioxopiperidin-3-yl)-1,3-dioxoisoindolin-4-yl)hexanamide (P-136)

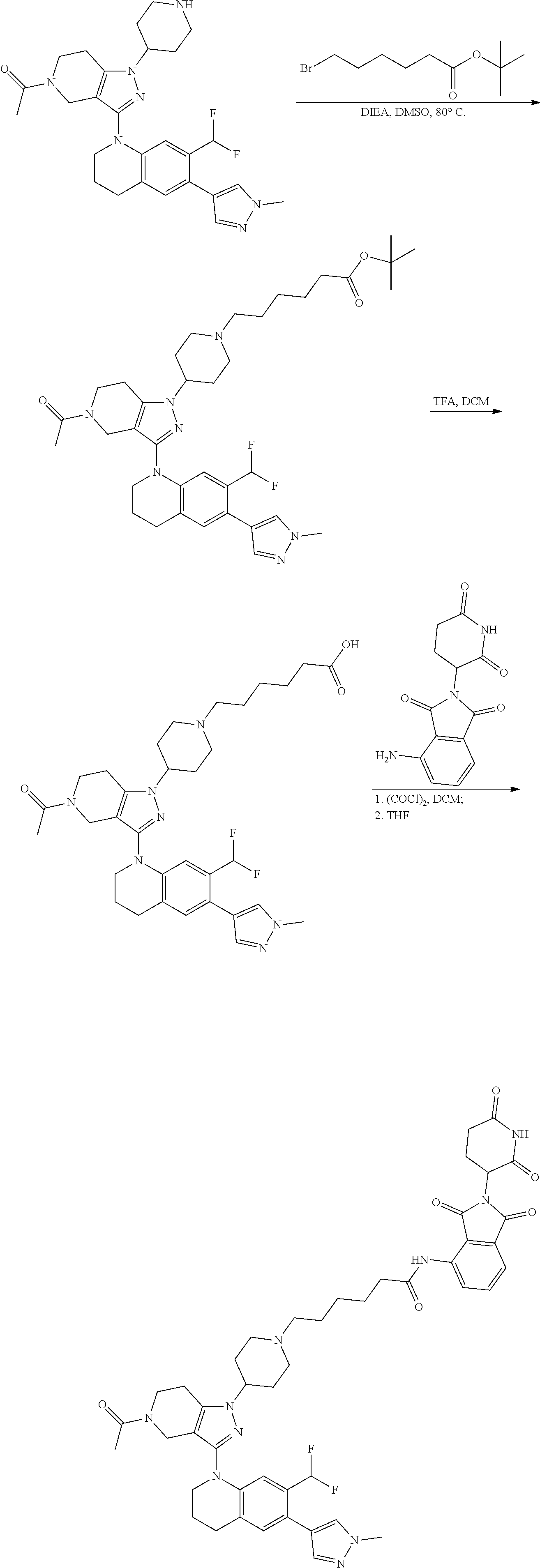 [SMILES: CC(=O)N1CCc2c(c(N3CCCc4cc(-c5cnn(C)c5)c(C(F)F)cc43)nn2C2CCNCC2)C1 ; CC(C)(C)OC(=O)CCCCCBr ; CC(=O)N1CCc2c(c(N3CCCc4cc(-c5cnn(C)c5)c(C(F)F)cc43)nn2C2CCN(CCCCCC(=O)OC(C)(C)C)CC2)C1 ; CC(=O)N1CCc2c(c(N3CCCc4cc(-c5cnn(C)c5)c(C(F)F)cc43)nn2C2CCN(CCCCCC(=O)O)CC2)C1 ; Nc1cccc2c1C(=O)N(C1CCC(=O)NC1=O)C2=O ; CC(=O)N1CCc2c(c(N3CCCc4cc(-c5cnn(C)c5)c(C(F)F)cc43)nn2C2CCN(CCCCCC(=O)Nc3cccc4c3C(=O)N(C3CCC(=O)NC3=O)C4=O)CC2)C1]

P-136 was synthesized following the standard procedure for preparing P-124 (28 mg, yield 99%). MS (ESI) m/z: 880.0 [M+H]⁺.

### Example 222. 3-(7-(Difluoromethyl)-6-(1-methyl-1H-pyrazol-4-yl)-3,4-dihydroquinolin-1(2H)-yl)-1-(1-(2-(4-((2-(2,6-dioxopiperidin-3-yl)-1,3-dioxoisoindolin-4-yl)methyl)piperazin-1-yl)acetyl)piperidin-4-yl)-N-methyl-1,4,6,7-tetrahydro-5H-pyrazolo[4,3-c]pyridine-5-carboxamide (P-137)

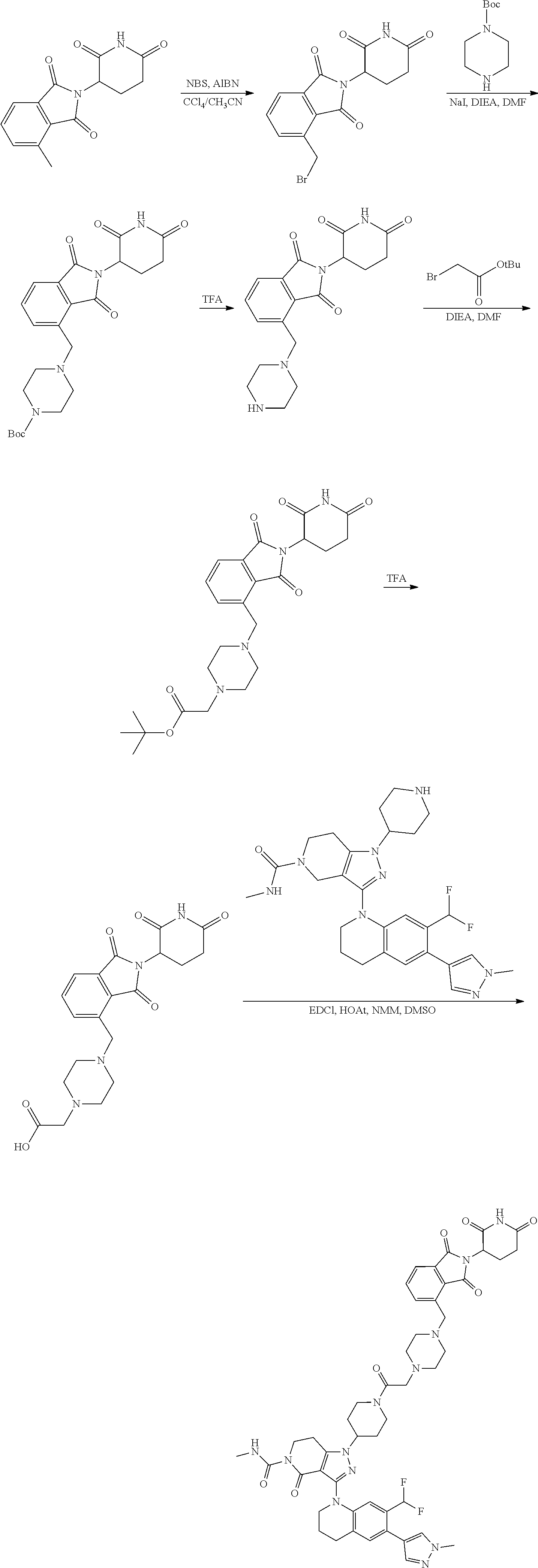

P-137 was synthesized following the standard procedure for preparing P-101 (40 mg, yield 99%). MS (ESI) m/z: 922.0 [M+H]⁺.

### Example 223. 4-((4-(2-(4-(5-Acetyl-3-(7-(difluoromethyl)-6-(1-methyl-1H-pyrazol-4-yl)-3,4-dihydroquinolin-1(2H)-yl)-4,5,6,7-tetrahydro-1H-pyrazolo[4,3-c]pyridin-1-yl)piperidin-1-yl)-2-oxoethyl)piperazin-1-yl)methyl)-2-(2,6-dioxopiperidin-3-yl)isoindoline-1,3-dione (P-138)

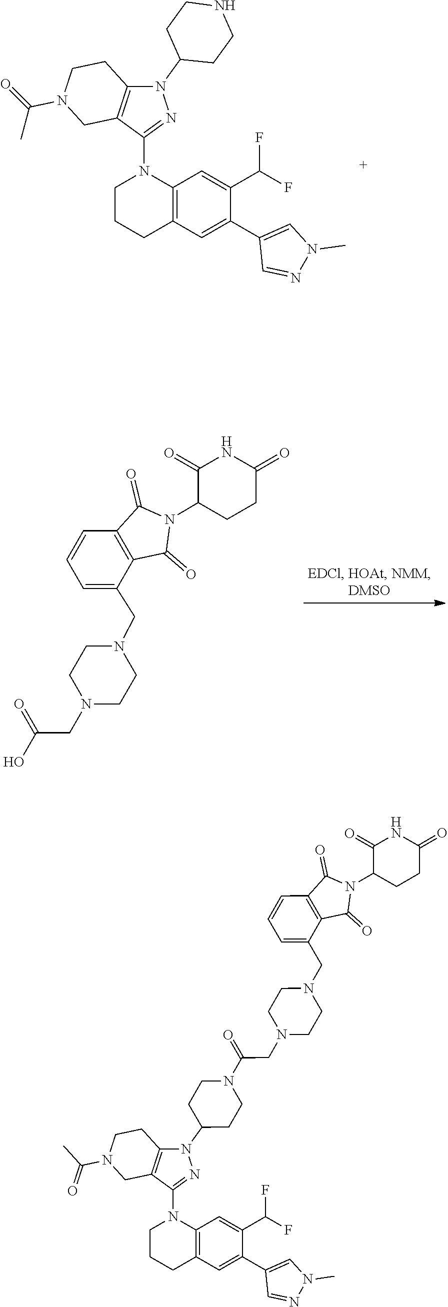

P-138 was synthesized following the standard procedure for preparing P-001 (34 mg, yield 86%). MS (ESI) m/z: 907.0 [M+H]⁺.

### Example 224. 4-(3-(4-(5-Acetyl-3-(7-(difluoromethyl)-6-(1-methyl-1H-pyrazol-4-yl)-3,4-dihydroquinolin-1(2H)-yl)-4,5,6,7-tetrahydro-1H-pyrazolo[4,3-c]pyridin-1-yl)-[1,4′-bipiperidin]-1′-yl)propoxy)-2-(2,6-dioxopiperidin-3-yl)isoindoline-1,3-dione (P-139)

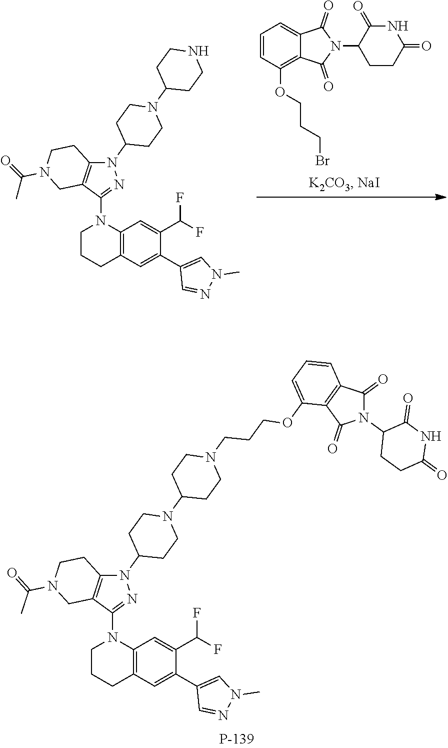 [SMILES: CC(=O)N1CCc2c(c(N3CCCc4cc(-c5cnn(C)c5)c(C(F)F)cc43)nn2C2CCN(C3CCNCC3)CC2)C1 ; O=C1CCC(N2C(=O)c3cccc(OCCCBr)c3C2=O)C(=O)N1 ; CC(=O)N1CCc2c(c(N3CCCc4cc(-c5cnn(C)c5)c(C(F)F)cc43)nn2C2CCN(C3CCN(CCCOc4cccc5c4C(=O)N(C4CCC(=O)NC4=O)C5=O)CC3)CC2)C1]

P-139 was synthesized following the standard procedure for preparing P-134 (39.8 mg, yield: 50%). MS (ESI) m/z: 908.0 [M+H]⁺.

### Example 225. 4-(3-(3-(4-(5-Acetyl-3-(7-(difluoromethyl)-6-(1-methyl-1H-pyrazol-4-yl)-3,4-dihydroquinolin-1(2H)-yl)-4,5,6,7-tetrahydro-1H-pyrazolo[4,3-c]pyridin-1-yl)piperidin-1-yl)azetidin-1-yl)propoxy)-2-(2,6-dioxopiperidin-3-yl)isoindoline-1,3-dione (P-140)

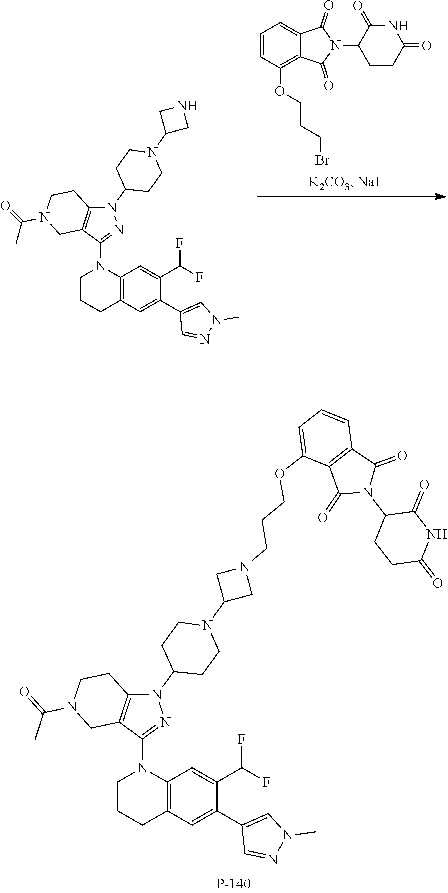 [SMILES: CC(=O)N1CCc2c(c(N3CCCc4cc(-c5cnn(C)c5)c(C(F)F)cc43)nn2C2CCN(C3CNC3)CC2)C1 ; O=C1CCC(N2C(=O)c3cccc(OCCCBr)c3C2=O)C(=O)N1 ; CC(=O)N1CCc2c(c(N3CCCc4cc(-c5cnn(C)c5)c(C(F)F)cc43)nn2C2CCN(C3CN(CCCOc4cccc5c4C(=O)N(C4CCC(=O)NC4=O)C5=O)C3)CC2)C1]

P-140 was synthesized following the standard procedure for preparing P-134 (17.7 mg, yield: 23%). MS (ESI) m/z: 880.0 [M+H]⁺.

### Example 226. 4-(((4-(2-(4-(5-Acetyl-3-(7-(difluoromethyl)-6-(1-methyl-1H-pyrazol-4-yl)-3,4-dihydroquinolin-1(2H)-yl)-4,5,6,7-tetrahydro-1H-pyrazolo[4,3-c]pyridin-1-yl)piperidin-1-yl)-2-oxoethyl)morpholin-2-yl)methyl)amino)-2-(2,6-dioxopiperidin-3-yl)isoindoline-1,3-dione (P-141)

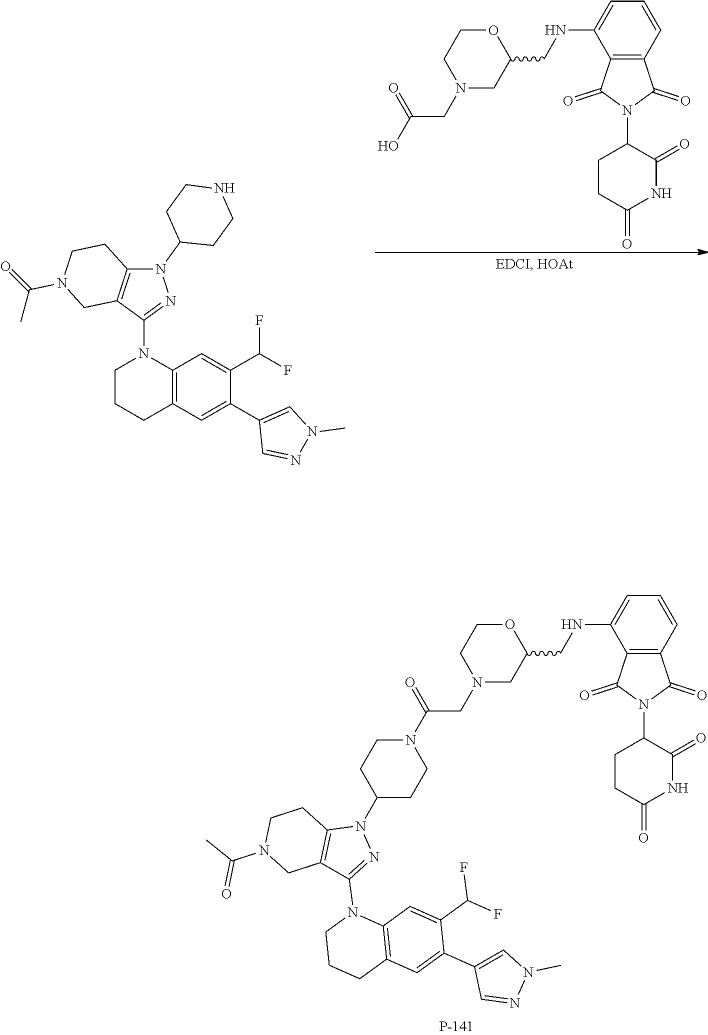 [SMILES: CC(=O)N1CCc2c(c(N3CCCc4cc(-c5cnn(C)c5)c(C(F)F)cc43)nn2C2CCNCC2)C1 ; O=C(O)CN1CCOC(CNc2cccc3c2C(=O)N(C2CCC(=O)NC2=O)C3=O)C1 ; CC(=O)N1CCc2c(c(N3CCCc4cc(-c5cnn(C)c5)c(C(F)F)cc43)nn2C2CCN(C(=O)CN3CCOC(CNc4cccc5c4C(=O)N(C4CCC(=O)NC4=O)C5=O)C3)CC2)C1]

P-141 was synthesized following the standard procedure for preparing P-001 (50 mg, yield: 92%). MS (ESI) m/z: 923.0 [M+H]⁺.

### Example 227. 4-(((4-(2-(4-(5-Acetyl-3-(7-(difluoromethyl)-6-(1-methyl-1H-pyrazol-4-yl)-3,4-dihydroquinolin-1(2H)-yl)-4,5,6,7-tetrahydro-1H-pyrazolo[4,3-c]pyridin-1-yl)piperidin-1-yl)-2-oxoethyl)morpholin-2-yl)methyl)amino)-2-(2,6-dioxopiperidin-3-yl)isoindoline-1,3-dione (P-142)

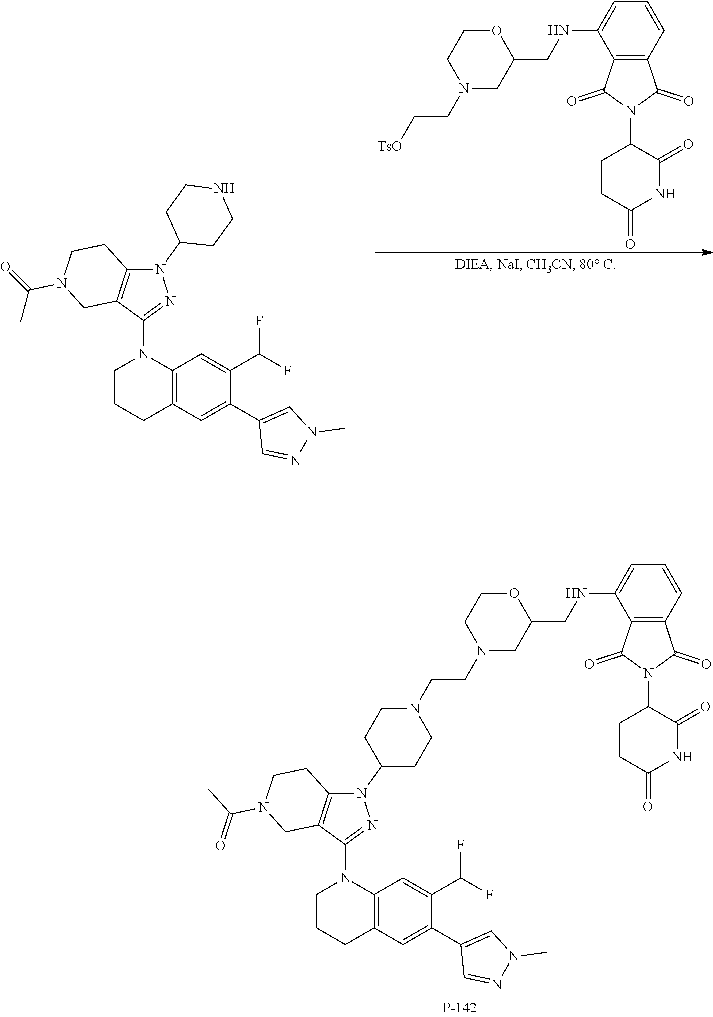 [SMILES: CC(=O)N1CCc2c(c(N3CCCc4cc(-c5cnn(C)c5)c(C(F)F)cc43)nn2C2CCNCC2)C1 ; Cc1ccc(S(=O)(=O)OCCN2CCOC(CNc3cccc4c3C(=O)N(C3CCC(=O)NC3=O)C4=O)C2)cc1 ; CC(=O)N1CCc2c(c(N3CCCc4cc(-c5cnn(C)c5)c(C(F)F)cc43)nn2C2CCN(CCN3CCOC(CNc4cccc5c4C(=O)N(C4CCC(=O)NC4=O)C5=O)C3)CC2)C1]

P-142 was synthesized following the standard procedure for preparing P-124 (24.9 mg, yield: 29%). MS (ESI) m/z: 909.0 [M+H]⁺.

### Example 228. 4-(((1-(2-(4-(5-Acetyl-3-(7-(difluoromethyl)-6-(1-methyl-1H-pyrazol-4-yl)-3,4-dihydroquinolin-1(2H)-yl)-4,5,6,7-tetrahydro-1H-pyrazolo[4,3-c]pyridin-1-yl)piperidin-1-yl)ethyl)piperidin-4-yl)methyl)amino)-2-(2,6-dioxopiperidin-3-yl)isoindoline-1,3-dione (P-143)

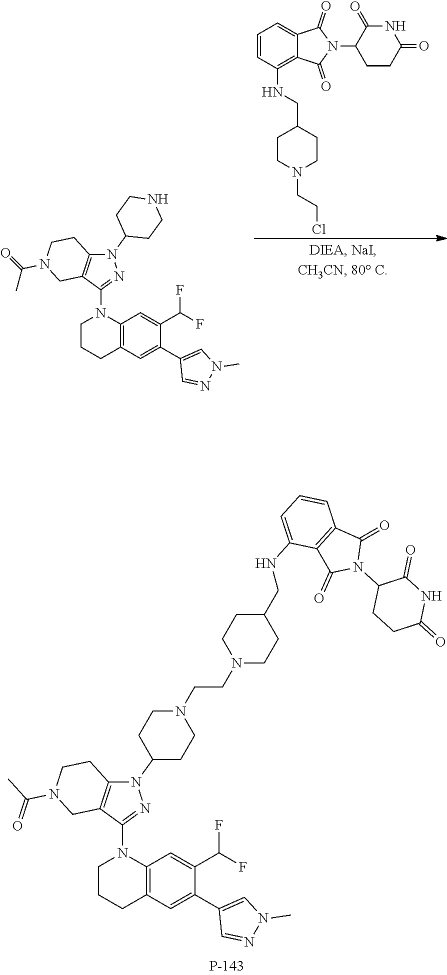 [SMILES: CC(=O)N1CCc2c(c(N3CCCc4cc(-c5cnn(C)c5)c(C(F)F)cc43)nn2C2CCNCC2)C1 ; O=C1CCC(N2C(=O)c3cccc(NCC4CCN(CCCl)CC4)c3C2=O)C(=O)N1 ; CC(=O)N1CCc2c(c(N3CCCc4cc(-c5cnn(C)c5)c(C(F)F)cc43)nn2C2CCN(CCN3CCC(CNc4cccc5c4C(=O)N(C4CCC(=O)NC4=O)C5=O)CC3)CC2)C1]

P-143 was synthesized following the standard procedure for preparing P-142 (2 mg, yield: 19%). MS (ESI) m/z: 906.9 [M+H]⁺.

### Example 229. 3-(7-(Difluoromethyl)-6-(1-methyl-1H-pyrazol-4-yl)-3,4-dihydroquinolin-1(2H)-yl)-1-(1-(2-(4-((2-(2,6-dioxopiperidin-3-yl)-1,3-dioxoisoindolin-4-yl)methyl)piperazin-1-yl)ethyl)piperidin-4-yl)-N-methyl-1,4,6,7-tetrahydro-5H-pyrazolo[4,3-c]pyridine-5-carboxamide (P-144)

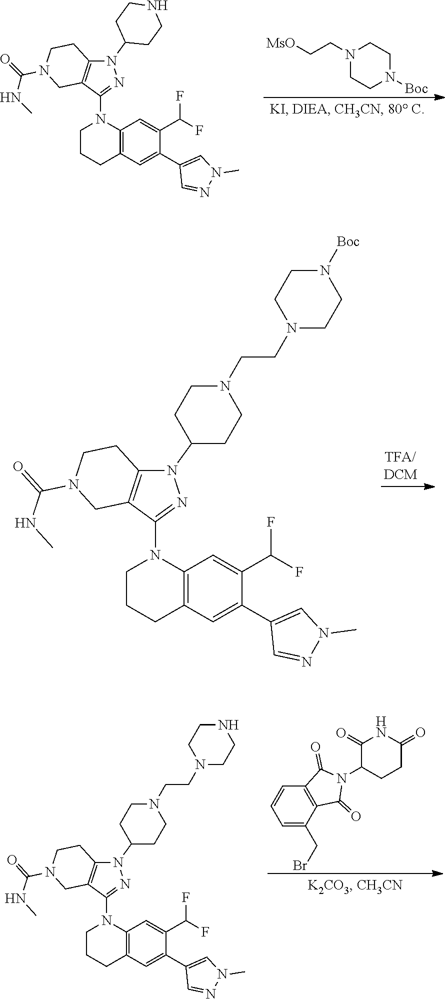 [SMILES: CNC(=O)N1CCc2c(c(N3CCCc4cc(-c5cnn(C)c5)c(C(F)F)cc43)nn2C2CCNCC2)C1 ; CC(C)(C)OC(=O)N1CCN(CCOS(C)(=O)=O)CC1 ; CNC(=O)N1CCc2c(c(N3CCCc4cc(-c5cnn(C)c5)c(C(F)F)cc43)nn2C2CCN(CCN3CCN(C(=O)OC(C)(C)C)CC3)CC2)C1 ; CNC(=O)N1CCc2c(c(N3CCCc4cc(-c5cnn(C)c5)c(C(F)F)cc43)nn2C2CCN(CCN3CCNCC3)CC2)C1 ; O=C1CCC(N2C(=O)c3cccc(CBr)c3C2=O)C(=O)N1]

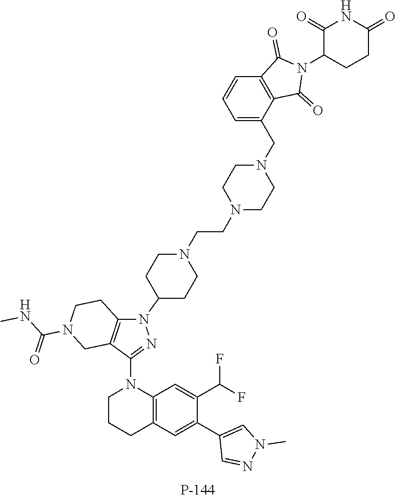 [SMILES: CNC(=O)N1CCc2c(c(N3CCCc4cc(-c5cnn(C)c5)c(C(F)F)cc43)nn2C2CCN(CCN3CCN(Cc4cccc5c4C(=O)N(C4CCC(=O)NC4=O)C5=O)CC3)CC2)C1]

P-144 was synthesized following the standard procedure for preparing P-156 (7.3 mg, yield: 49%). MS (ESI) m/z: 908.0 [M+H]⁺.

### Example 230. 4-((4-(2-(4-(5-Acetyl-3-(7-(difluoromethyl)-6-(1-methyl-1H-pyrazol-4-yl)-3,4-dihydroquinolin-1(2H)-yl)-4,5,6,7-tetrahydro-1H-pyrazolo[4,3-c]pyridin-1-yl)piperidin-1-yl)ethyl)piperazin-1-yl)methyl)-2-(2,6-dioxopiperidin-3-yl)isoindoline-1,3-dione (P-145)

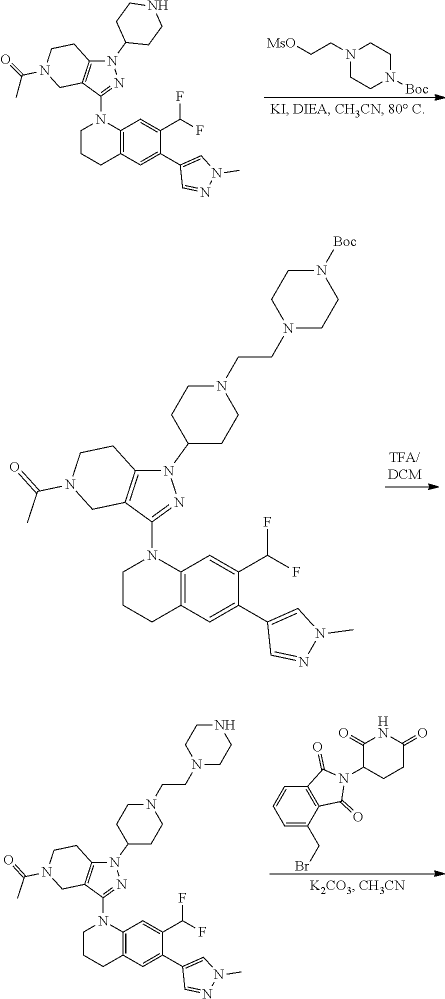 [SMILES: CC(=O)N1CCc2c(c(N3CCCc4cc(-c5cnn(C)c5)c(C(F)F)cc43)nn2C2CCNCC2)C1 ; CC(C)(C)OC(=O)N1CCN(CCOS(C)(=O)=O)CC1 ; CC(=O)N1CCc2c(c(N3CCCc4cc(-c5cnn(C)c5)c(C(F)F)cc43)nn2C2CCN(CCN3CCN(C(=O)OC(C)(C)C)CC3)CC2)C1 ; CC(=O)N1CCc2c(c(N3CCCc4cc(-c5cnn(C)c5)c(C(F)F)cc43)nn2C2CCN(CCN3CCNCC3)CC2)C1 ; O=C1CCC(N2C(=O)c3cccc(CBr)c3C2=O)C(=O)N1]

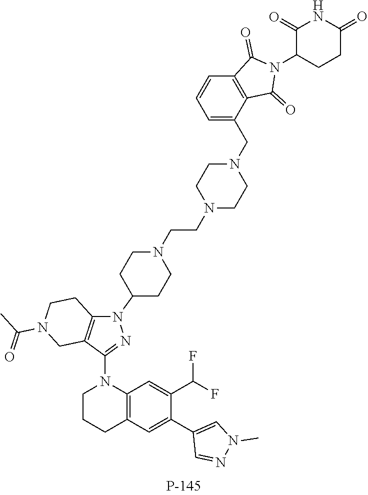 [SMILES: CC(=O)N1CCc2c(c(N3CCCc4cc(-c5cnn(C)c5)c(C(F)F)cc43)nn2C2CCN(CCN3CCN(Cc4cccc5c4C(=O)N(C4CCC(=O)NC4=O)C5=O)CC3)CC2)C1]

P-145 was synthesized following the standard procedure for preparing P-156 (7.5 mg, yield: 40%). MS (ESI) m/z: 892.9 [M+H]⁺.

### Example 231. 4-(3-(4-(4-(5-Acetyl-3-(7-(difluoromethyl)-6-(1-methyl-1H-pyrazol-4-yl)-3,4-dihydroquinolin-1(2H)-yl)-4,5,6,7-tetrahydro-1H-pyrazolo[4,3-c]pyridin-1-yl)piperidin-1-yl)-4-oxobutyl)azetidin-1-yl)-2-(2,6-dioxopiperidin-3-yl)isoindoline-1,3-dione (P-146)

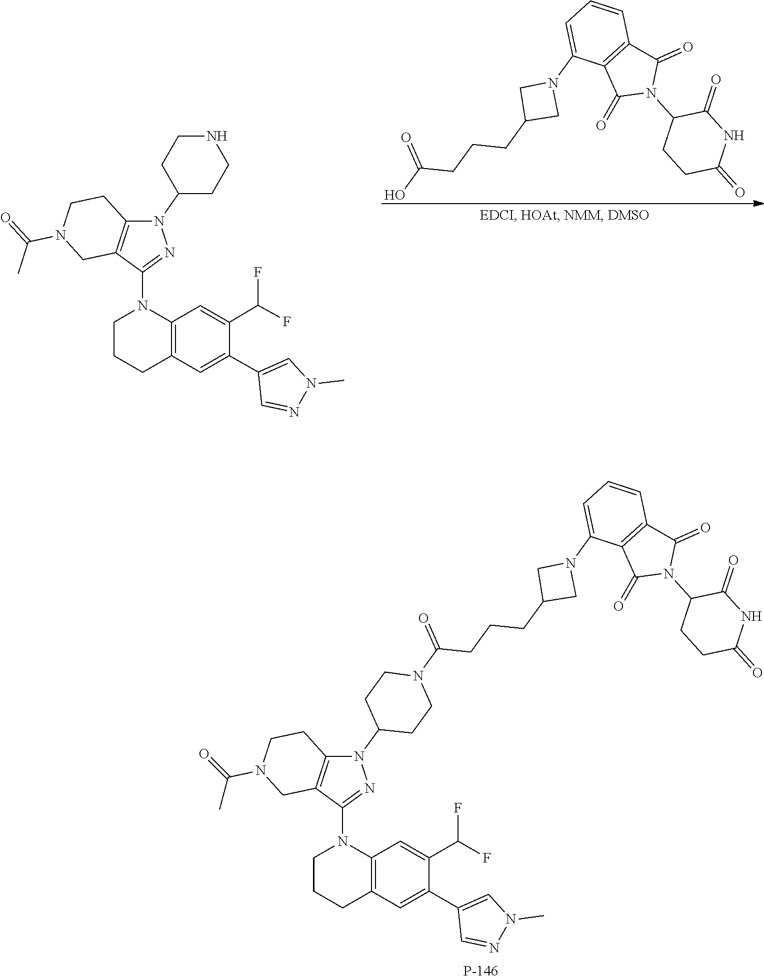 [SMILES: CC(=O)N1CCc2c(c(N3CCCc4cc(-c5cnn(C)c5)c(C(F)F)cc43)nn2C2CCNCC2)C1 ; O=C(O)CCCC1CN(c2cccc3c2C(=O)N(C2CCC(=O)NC2=O)C3=O)C1 ; CC(=O)N1CCc2c(c(N3CCCc4cc(-c5cnn(C)c5)c(C(F)F)cc43)nn2C2CCN(C(=O)CCCC3CN(c4cccc5c4C(=O)N(C4CCC(=O)NC4=O)C5=O)C3)CC2)C1]

P-146 was synthesized following the standard procedure for preparing P-001 (105 mg, yield: 78%). MS (ESI) m/z: 891.9 [M+H]⁺.

### Example 232. 4-(3-(1-(2-(4-(5-Acetyl-3-(7-(difluoromethyl)-6-(1-methyl-1H-pyrazol-4-yl)-3,4-dihydroquinolin-1(2H)-yl)-4,5,6,7-tetrahydro-1H-pyrazolo[4,3-c]pyridin-1-yl)piperidin-1-yl)-2-oxoethyl)piperidin-4-yl)propyl)-2-(2,6-dioxopiperidin-3-yl)isoindoline-1,3-dione (P-147)

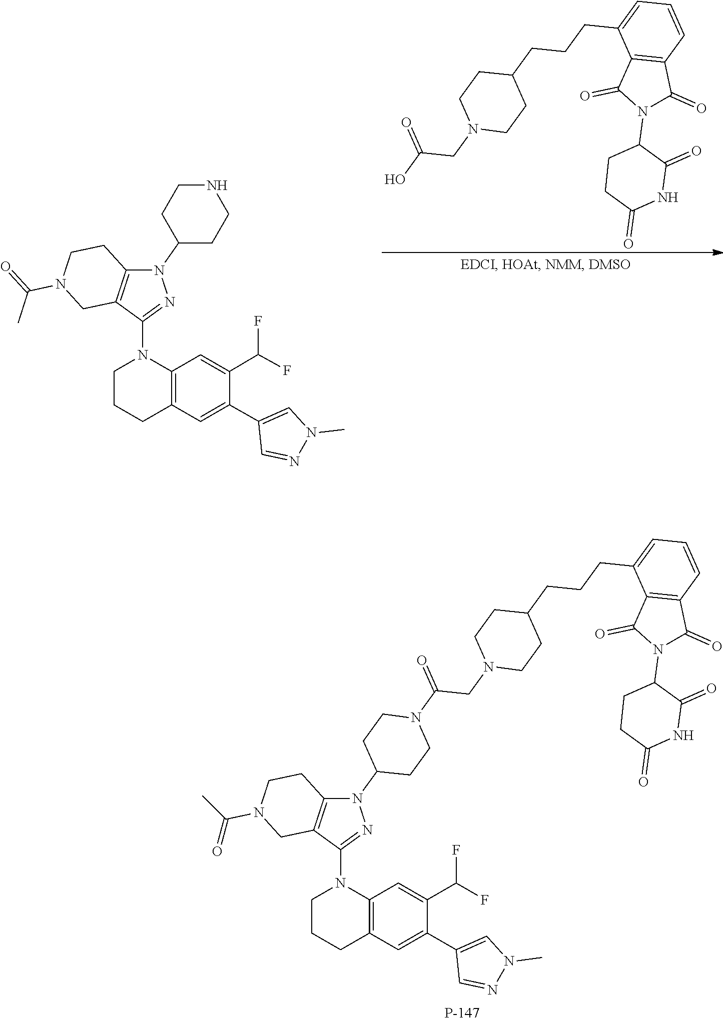

P-147 was synthesized following the standard procedure for preparing P-001 (5 mg, yield: 6%). MS (ESI) m/z: 934.0 [M+H]⁺.

### Example 233. 4-(2-(1-(3-(4-(5-Acetyl-3-(7-(difluoromethyl)-6-(1-methyl-1H-pyrazol-4-yl)-3,4-dihydroquinolin-1(2H)-yl)-4,5,6,7-tetrahydro-1H-pyrazolo[4,3-c]pyridin-1-yl)piperidin-1-yl)-3-oxopropyl)piperidin-4-yl)ethyl)-2-(2,6-dioxopiperidin-3-yl)isoindoline-1,3-dione (P-148)

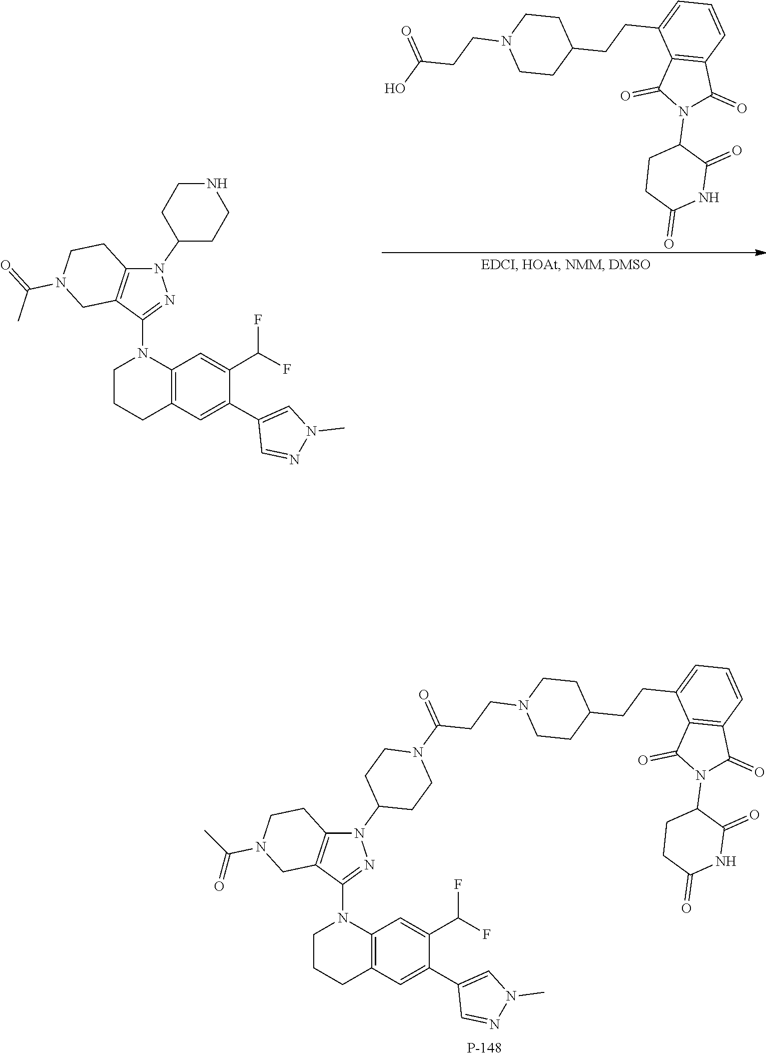 [SMILES: CC(=O)N1CCc2c(c(N3CCCc4cc(-c5cnn(C)c5)c(C(F)F)cc43)nn2C2CCNCC2)C1 ; O=C(O)CCN1CCC(CCc2cccc3c2C(=O)N(C2CCC(=O)NC2=O)C3=O)CC1 ; CC(=O)N1CCc2c(c(N3CCCc4cc(-c5cnn(C)c5)c(C(F)F)cc43)nn2C2CCN(C(=O)CCN3CCC(CCc4cccc5c4C(=O)N(C4CCC(=O)NC4=O)C5=O)CC3)CC2)C1]

P-148 was synthesized following the standard procedure for preparing P-001 (39 mg, yield: 62%). MS (ESI) m/z: 934.0 [M+H]⁺.

### Example 234. 4-(3-(4-((4-(5-Acetyl-3-(7-(difluoromethyl)-6-(1-methyl-1H-pyrazol-4-yl)-3,4-dihydroquinolin-1(2H)-yl)-4,5,6,7-tetrahydro-1H-pyrazolo[4,3-c]pyridin-1-yl)piperidin-1-yl)methyl)piperidin-1-yl)propyl)-2-(2,6-dioxopiperidin-3-yl)isoindoline-1,3-dione (P-149)

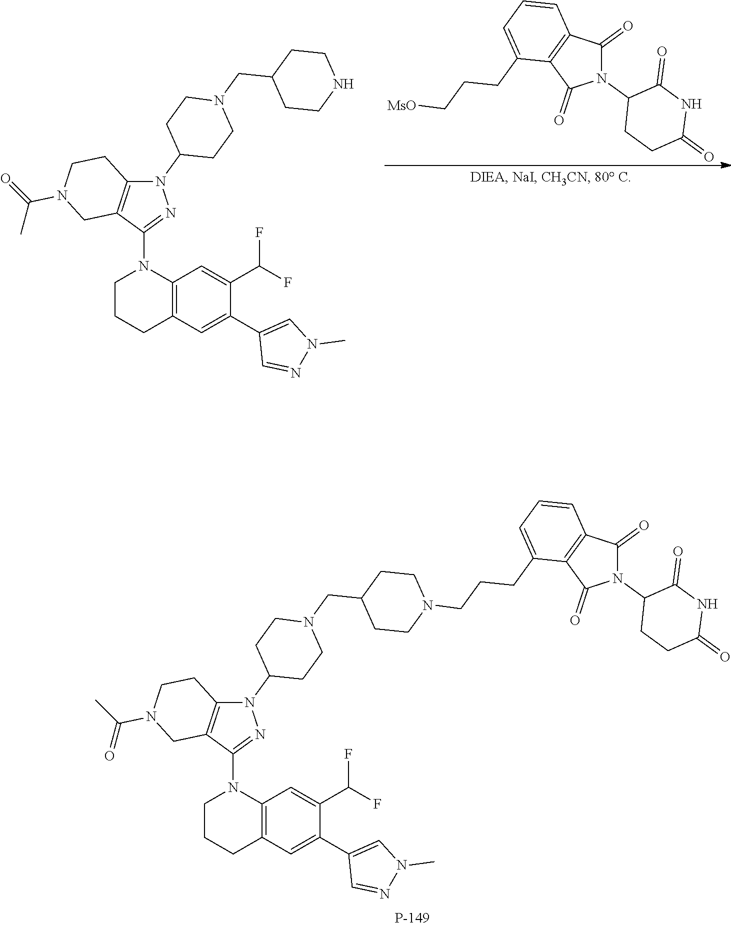

P-149 was synthesized following the standard procedure for preparing P-142 (16 mg, yield: 17%). MS (ESI) m/z: 906.0 [M+H]⁺.

### Example 235. 4-(3-(4-((4-(5-Acetyl-3-(7-(difluoromethyl)-6-(1-methyl-1H-pyrazol-4-yl)-3,4-dihydroquinolin-1(2H)-yl)-4,5,6,7-tetrahydro-1H-pyrazolo[4,3-c]pyridin-1-yl)piperidin-1-yl)methyl)piperidin-1-yl)prop-1-yn-1-yl)-2-(2,6-dioxopiperidin-3-yl)isoindoline-1,3-dione (P-150)

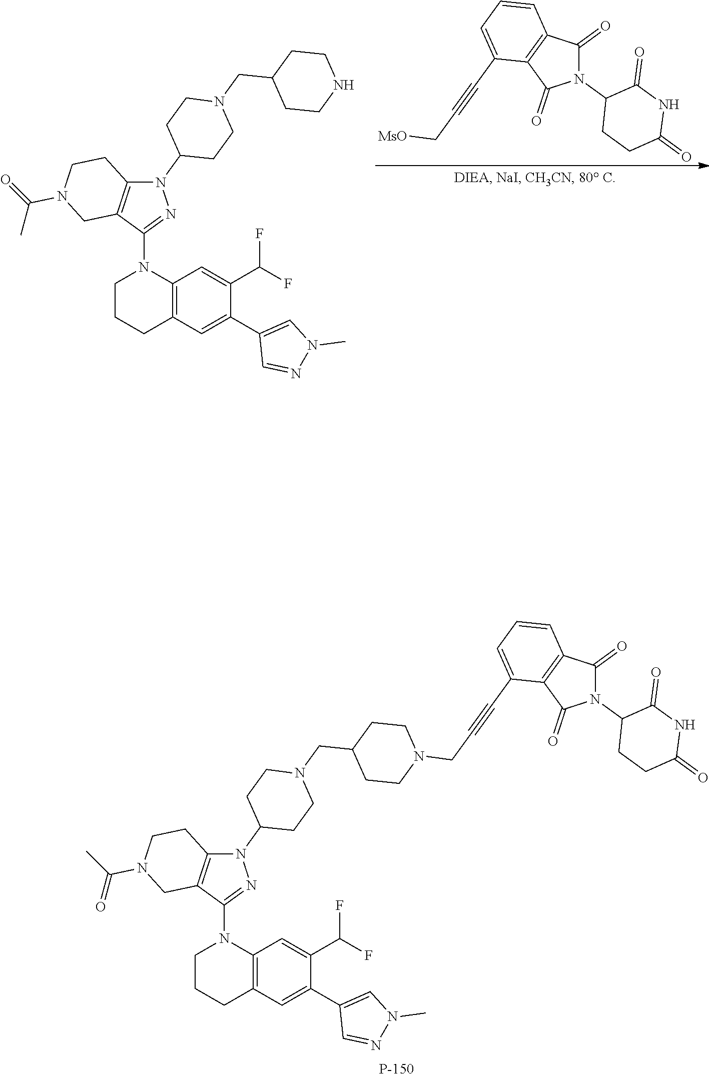

P-150 was synthesized following the standard procedure for preparing P-142 (12 mg, yield: 26%). MS (ESI) m/z: 901.9 [M+H]⁺.

### Example 236. 3-(5-((6-(4-(5-Acetyl-3-(7-(difluoromethyl)-6-(1-methyl-1H-pyrazol-4-yl)-3,4-dihydroquinolin-1(2H)-yl)-4,5,6,7-tetrahydro-1H-pyrazolo[4,3-c]pyridin-1-yl)piperidin-1-yl)-6-oxohexyl)amino)-4-oxobenzo[d][1,2,3]triazin-3(4H)-yl)piperidine-2,6-dione (P-151)

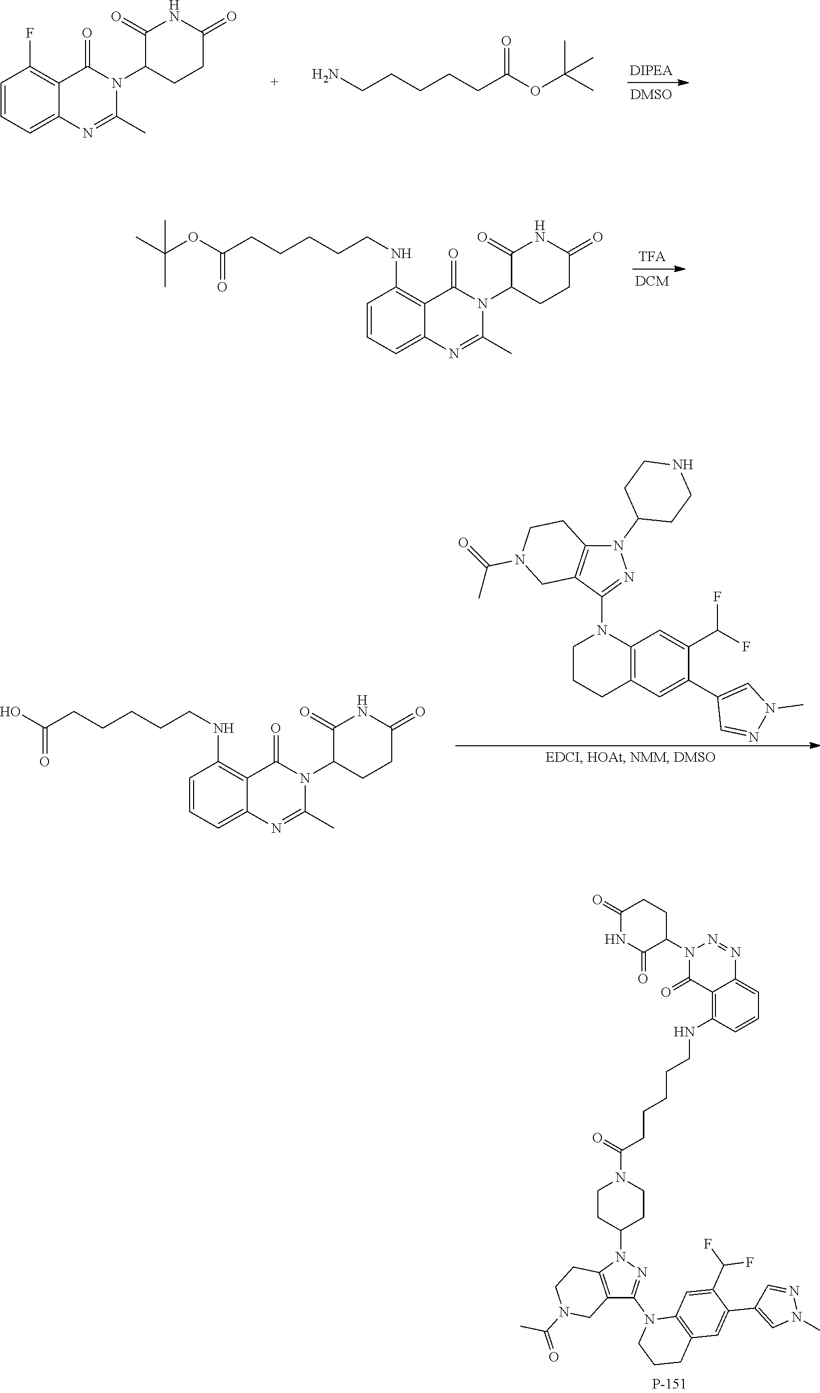 [SMILES: Cc1nc2cccc(F)c2c(=O)n1C1CCC(=O)NC1=O ; CC(C)(C)OC(=O)CCCCCN ; Cc1nc2cccc(NCCCCCC(=O)OC(C)(C)C)c2c(=O)n1C1CCC(=O)NC1=O ; Cc1nc2cccc(NCCCCCC(=O)O)c2c(=O)n1C1CCC(=O)NC1=O ; CC(=O)N1CCc2c(c(N3CCCc4cc(-c5cnn(C)c5)c(C(F)F)cc43)nn2C2CCNCC2)C1 ; CC(=O)N1CCc2c(c(N3CCCc4cc(-c5cnn(C)c5)c(C(F)F)cc43)nn2C2CCN(C(=O)CCCCCNc3cccc4nnn(C5CCC(=O)NC5=O)c(=O)c34)CC2)C1]

P-151 was synthesized following the standard procedure for preparing P-001 (8.8 mg, yield: 40%). MS (ESI) m/z: 880.0 [M+H]⁺.

### Example 237. 3-(5-((7-(4-(5-Acetyl-3-(7-(difluoromethyl)-6-(1-methyl-1H-pyrazol-4-yl)-3,4-dihydroquinolin-1(2H)-yl)-4,5,6,7-tetrahydro-1H-pyrazolo[4,3-c]pyridin-1-yl)piperidin-1-yl)-7-oxoheptyl)amino)-2-methyl-4-oxoquinazolin-3(4H)-yl)piperidine-2,6-dione (P-152)

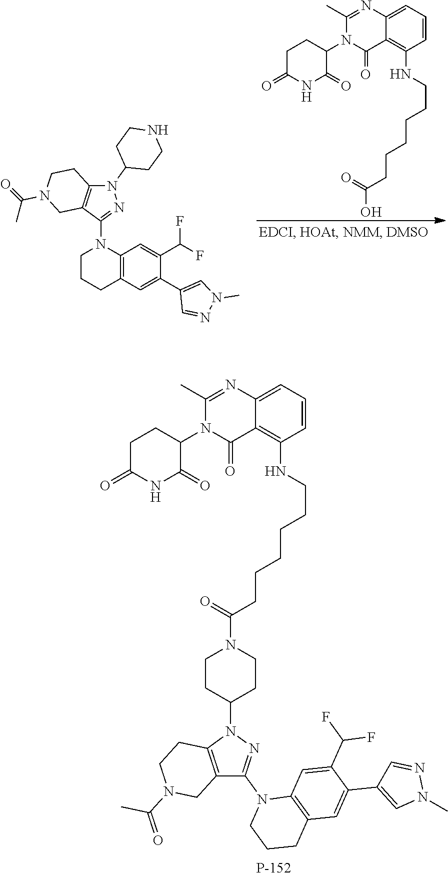 [SMILES: CC(=O)N1CCc2c(c(N3CCCc4cc(-c5cnn(C)c5)c(C(F)F)cc43)nn2C2CCNCC2)C1 ; Cc1nc2cccc(NCCCCCCC(=O)O)c2c(=O)n1C1CCC(=O)NC1=O ; CC(=O)N1CCc2c(c(N3CCCc4cc(-c5cnn(C)c5)c(C(F)F)cc43)nn2C2CCN(C(=O)CCCCCCNc3cccc4nc(C)n(C5CCC(=O)NC5=O)c(=O)c34)CC2)C1]

P-152 was synthesized following the standard procedure for preparing P-151 (13 mg, yield: 59%). MS (ESI) m/z: 907.0 [M+H]⁺.

### Example 238. 3-(5-((7-(4-(5-Acetyl-3-(7-(difluoromethyl)-6-(1-methyl-1H-pyrazol-4-yl)-3,4-dihydroquinolin-1(2H)-yl)-4,5,6,7-tetrahydro-1H-pyrazolo[4,3-c]pyridin-1-yl)piperidin-1-yl)-7-oxoheptyl)amino)-4-oxobenzo[d][1,2,3]triazin-3(4H)-yl)piperidine-2,6-dione (P-153)

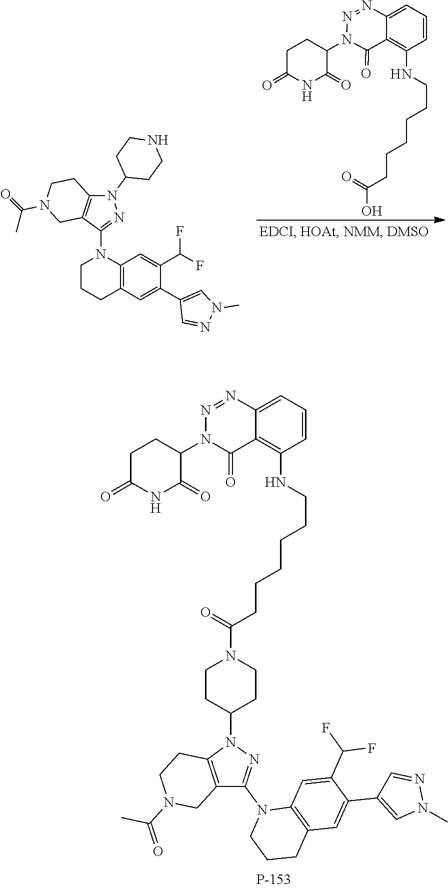 [SMILES: CC(=O)N1CCc2c(c(N3CCCc4cc(-c5cnn(C)c5)c(C(F)F)cc43)nn2C2CCNCC2)C1 ; O=C(O)CCCCCCNc1cccc2nnn(C3CCC(=O)NC3=O)c(=O)c12 ; CC(=O)N1CCc2c(c(N3CCCc4cc(-c5cnn(C)c5)c(C(F)F)cc43)nn2C2CCN(C(=O)CCCCCCNc3cccc4nnn(C5CCC(=O)NC5=O)c(=O)c34)CC2)C1]

P-153 was synthesized following the standard procedure for preparing P-151 (9 mg, yield: 40%). MS (ESI) m/z: 894.0 [M+H]⁺.

### Example 239. 3-(5-((5-(4-(5-Acetyl-3-(7-(difluoromethyl)-6-(1-methyl-1H-pyrazol-4-yl)-3,4-dihydroquinolin-1(2H)-yl)-4,5,6,7-tetrahydro-1H-pyrazolo[4,3-c]pyridin-1-yl)piperidin-1-yl)-5-oxopentyl)amino)-2-methyl-4-oxoquinazolin-3(4H)-yl)piperidine-2,6-dione (P-154)

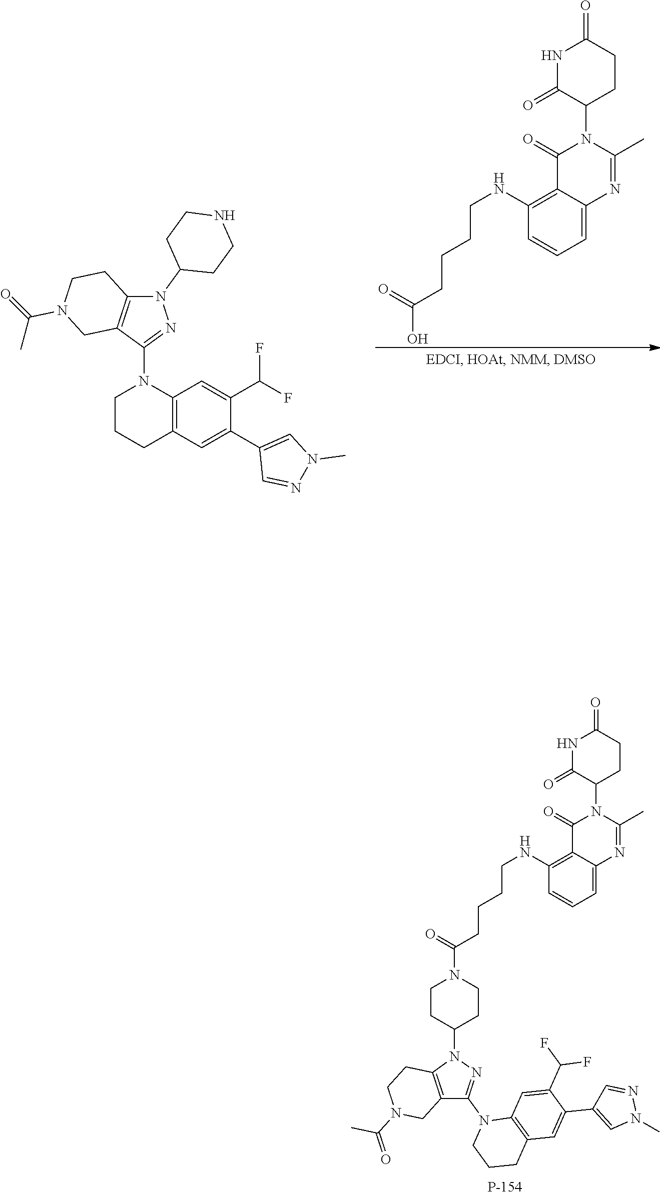 [SMILES: CC(=O)N1CCc2c(c(N3CCCc4cc(-c5cnn(C)c5)c(C(F)F)cc43)nn2C2CCNCC2)C1 ; Cc1nc2cccc(NCCCCC(=O)O)c2c(=O)n1C1CCC(=O)NC1=O ; CC(=O)N1CCc2c(c(N3CCCc4cc(-c5cnn(C)c5)c(C(F)F)cc43)nn2C2CCN(C(=O)CCCCNc3cccc4nc(C)n(C5CCC(=O)NC5=O)c(=O)c34)CC2)C1]

P-154 was synthesized following the standard procedure for preparing P-151 (11 mg, yield: 40%). MS (ESI) m/z: 879.0 [M+H]⁺.

### Example 240. 3-(5-((5-(4-(5-Acetyl-3-(7-(difluoromethyl)-6-(1-methyl-1H-pyrazol-4-yl)-3,4-dihydroquinolin-1(2H)-yl)-4,5,6,7-tetrahydro-1H-pyrazolo[4,3-c]pyridin-1-yl)piperidin-1-yl)-5-oxopentyl)amino)-4-oxobenzo[d][1,2,3]triazin-3(4H)-yl)piperidine-2,6-dione (P-155)

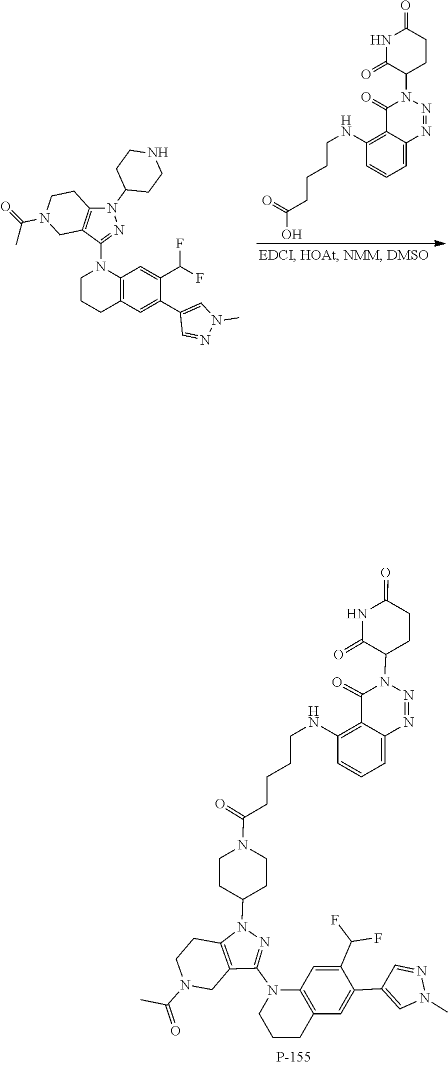

P-155 was synthesized following the standard procedure for preparing P-151 (13 mg, yield: 43%). MS (ESI) ink: 865.9 [M+H]⁺.

### Example 241. 4-((3-(3-(4-(5-Acetyl-3-(7-(difluoromethyl)-6-(1-methyl-1H-pyrazol-4-yl)-3,4-dihydroquinolin-1(2H)-yl)-4,5,6,7-tetrahydro-1H-pyrazolo[4,3-c]pyridin-1-yl)piperidin-1-yl)-3-oxopropyl)azetidin-1-yl)methyl)-2-(2,6-dioxopiperidin-3-yl)isoindoline-1,3-dione (P-156)

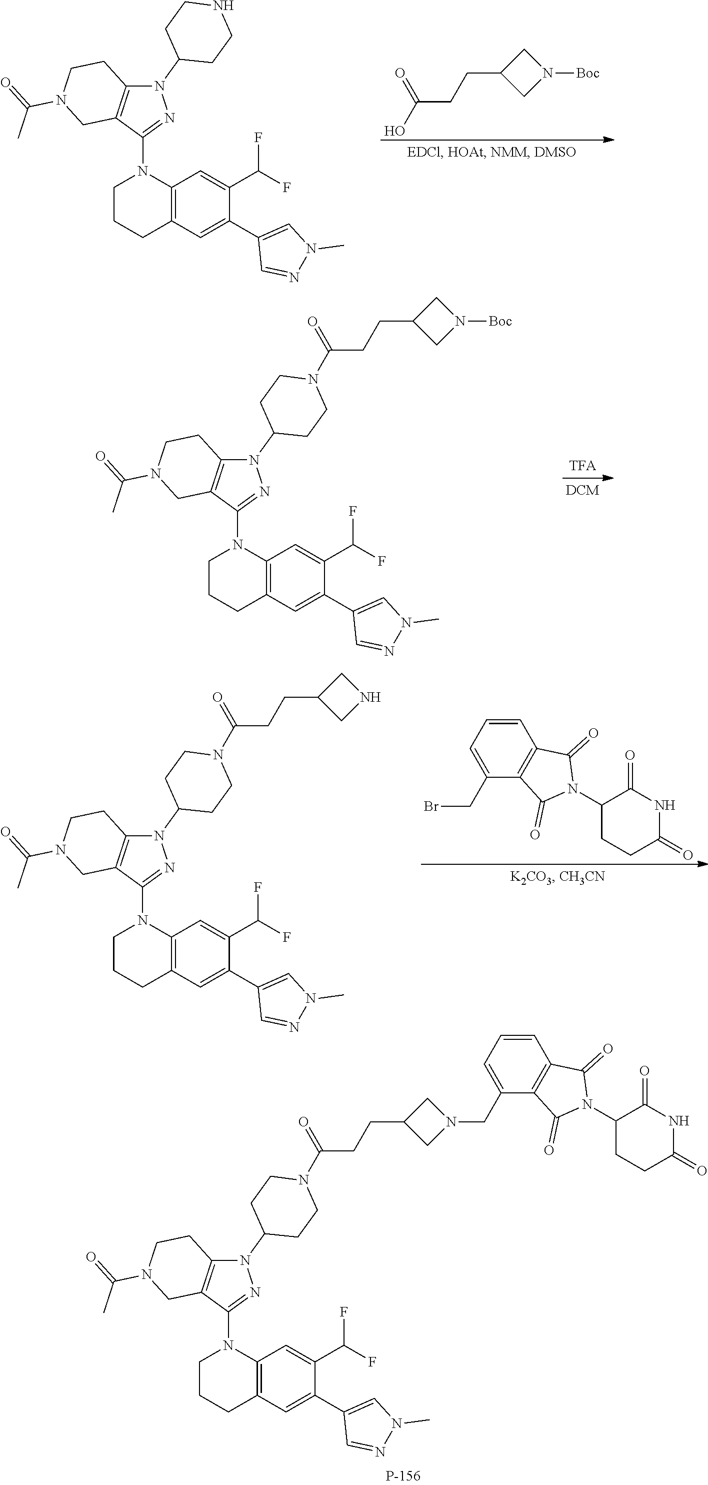

### Step 1. Synthesis of 1-(4-(5-acetyl-3-(7-(difluoromethyl)-6-(1-methyl-1H-pyrazol-4-yl)-3,4-dihydroquinolin-1(2H)-yl)-4,5,6,7-tetrahydro-1H-pyrazolo[4,3-c]pyridin-1-yl)piperidin-1-yl)-3-(azetidin-3-yl)propan-1-one

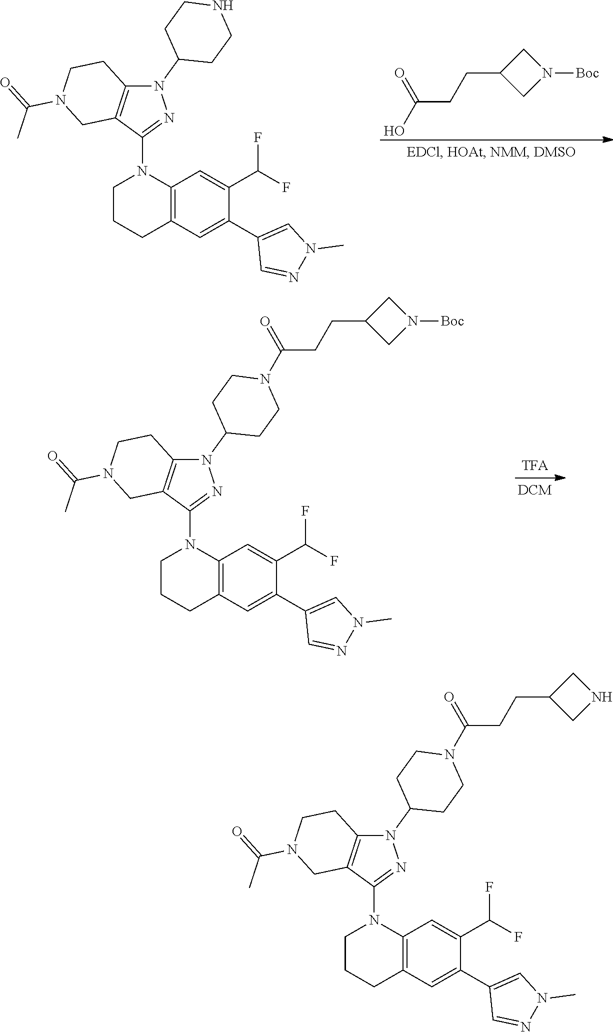 [SMILES: CC(=O)N1CCc2c(c(N3CCCc4cc(-c5cnn(C)c5)c(C(F)F)cc43)nn2C2CCNCC2)C1 ; CC(C)(C)OC(=O)N1CC(CCC(=O)O)C1 ; CC(=O)N1CCc2c(c(N3CCCc4cc(-c5cnn(C)c5)c(C(F)F)cc43)nn2C2CCN(C(=O)CCC3CN(C(=O)OC(C)(C)C)C3)CC2)C1 ; CC(=O)N1CCc2c(c(N3CCCc4cc(-c5cnn(C)c5)c(C(F)F)cc43)nn2C2CCN(C(=O)CCC3CNC3)CC2)C1]

A mixture of 1-(3-(7-(difluoromethyl)-6-(1-methyl-1H-pyrazol-4-yl)-3,4-dihydroquinolin-1(2H)-yl)-1-(piperidin-4-yl)-1,4,6,7-tetrahydro-5H-pyrazolo[4,3-c]pyridin-5-yl)ethan-1-one (180 mg, 0.354 mmol), 3-(1-(tert-butoxycarbonyl)azetidin-3-yl)propanoic acid (81 mg, 0.354 mmol), EDCI (102 mg, 0.531 mmol), HOAt (72 mg, 0.531 mmol) and NMM (107 mg, 1.062 mmol) in DMSO (3 mL) was stirred at room temperature overnight. The reaction mixture was diluted with EtOAc, washed with water, dried over Na₂SO₄, filtered and concentrated. The residue was purified by silica gel column chromatography to give a crude product (110 mg), which was dissolved in DCM (5 mL) followed by addition of TFA (1 mL). The resulting mixture was stirred at room temperature for 3 h, before it was concentrated. The resulting residue was purified by Prep-TLC to give desired product (23 mg, 10% for 2 steps) which was used directly in the next step.

### Step 2. Synthesis of 4-((3-(3-(4-(5-acetyl-3-(7-(difluoromethyl)-6-(1-methyl-1H-pyrazol-4-yl)-3,4-dihydroquinolin-1(2H)-yl)-4,5,6,7-tetrahydro-1H-pyrazolo[4,3-c]pyridin-1-yl)piperidin-1-yl)-3-oxopropyl)azetidin-1-yl)methyl)-2-(2,6-dioxopiperidin-3-yl)isoindoline-1,3-dione

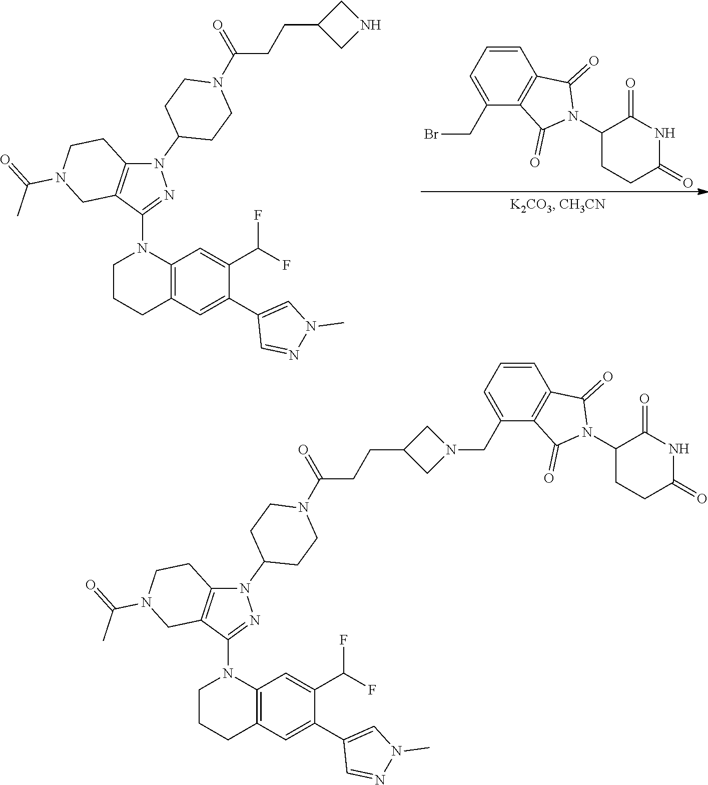 [SMILES: CC(=O)N1CCc2c(c(N3CCCc4cc(-c5cnn(C)c5)c(C(F)F)cc43)nn2C2CCN(C(=O)CCC3CNC3)CC2)C1 ; O=C1CCC(N2C(=O)c3cccc(CBr)c3C2=O)C(=O)N1 ; CC(=O)N1CCc2c(c(N3CCCc4cc(-c5cnn(C)c5)c(C(F)F)cc43)nn2C2CCN(C(=O)CCC3CN(Cc4cccc5c4C(=O)N(C4CCC(=O)NC4=O)C5=O)C3)CC2)C1]

P-156 was synthesized following the standard procedure for preparing P-142 (20 mg, yield: 61%). MS (ESI) m/z: 891.9 [M+H]⁺.

### Example 242. 4-(2-(1-(2-(4-(5-Acetyl-3-(7-(difluoromethyl)-6-(1-methyl-1H-pyrazol-4-yl)-3,4-dihydroquinolin-1(2H)-yl)-4,5,6,7-tetrahydro-1H-pyrazolo[4,3-c]pyridin-1-yl)piperidin-1-yl)ethyl)piperidin-4-yl)ethyl)-2-(2,6-dioxopiperidin-3-yl)isoindoline-1,3-dione (P-157)

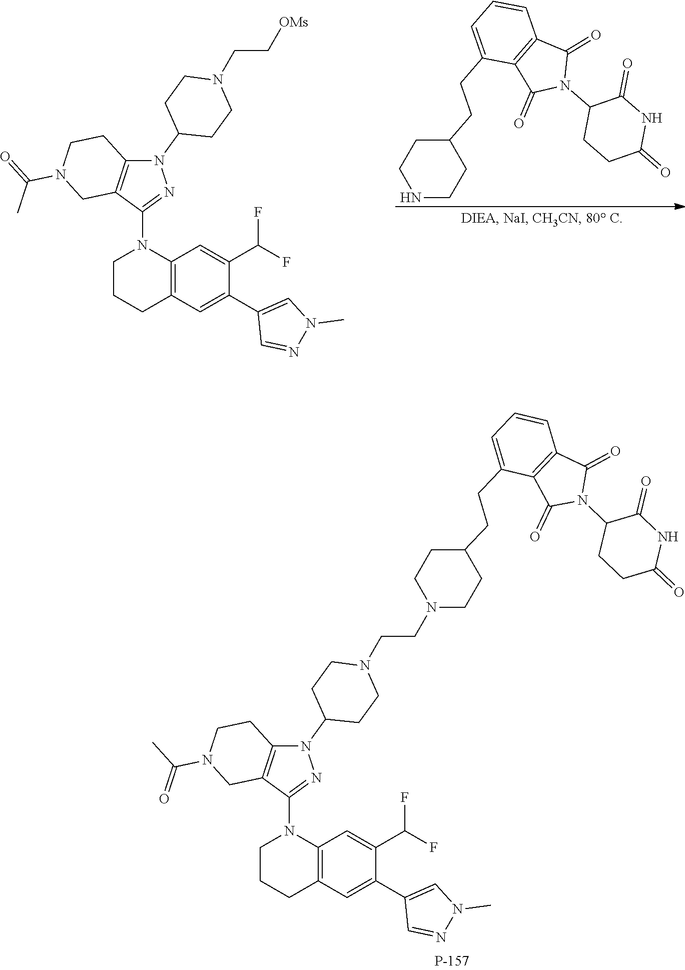 [SMILES: CC(=O)N1CCc2c(c(N3CCCc4cc(-c5cnn(C)c5)c(C(F)F)cc43)nn2C2CCN(CCOS(C)(=O)=O)CC2)C1 ; O=C1CCC(N2C(=O)c3cccc(CCC4CCNCC4)c3C2=O)C(=O)N1 ; CC(=O)N1CCc2c(c(N3CCCc4cc(-c5cnn(C)c5)c(C(F)F)cc43)nn2C2CCN(CCN3CCC(CCc4cccc5c4C(=O)N(C4CCC(=O)NC4=O)C5=O)CC3)CC2)C1]

P-157 was synthesized following the standard procedure for preparing P-142 (13 mg, yield: 16%). MS (ESI) m/z: 905.9 [M+H]⁺.

### Example 243. 4-(((1-(2-(4-(5-Acetyl-3-(7-(difluoromethyl)-6-(1-methyl-1H-pyrazol-4-yl)-3,4-dihydroquinolin-1(2H)-yl)-4,5,6,7-tetrahydro-1H-pyrazolo[4,3-c]pyridin-1-yl)piperidin-1-yl)-2-oxoethyl)azetidin-3-yl)methyl)amino)-2-(2,6-dioxopiperidin-3-yl)isoindoline-1,3-dione (P-158)

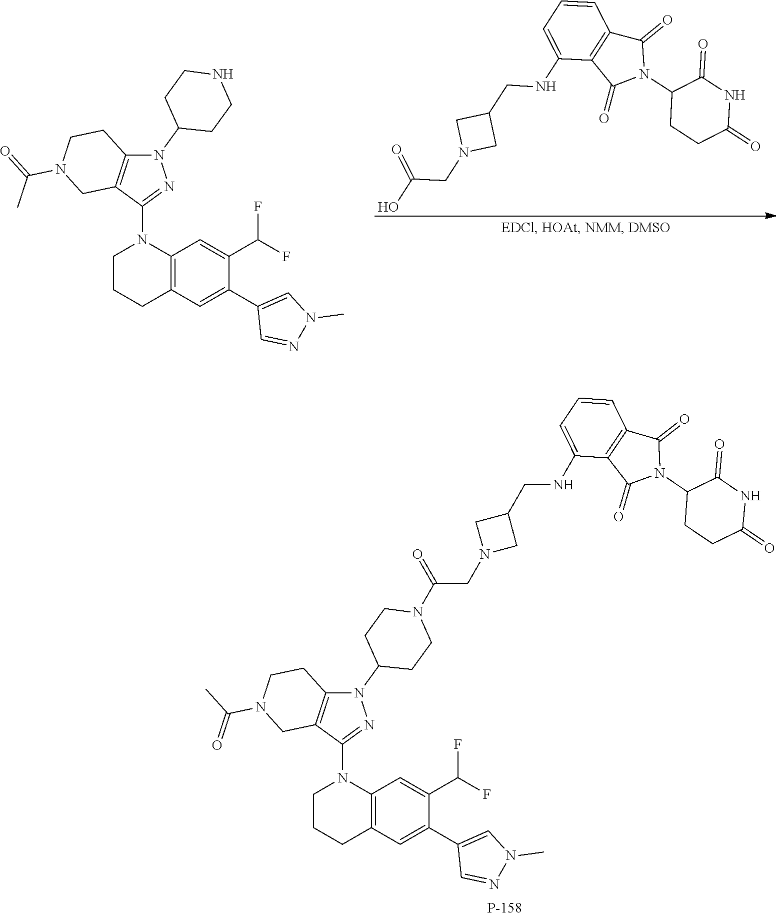 [SMILES: CC(=O)N1CCc2c(c(N3CCCc4cc(-c5cnn(C)c5)c(C(F)F)cc43)nn2C2CCNCC2)C1 ; O=C(O)CN1CC(CNc2cccc3c2C(=O)N(C2CCC(=O)NC2=O)C3=O)C1 ; CC(=O)N1CCc2c(c(N3CCCc4cc(-c5cnn(C)c5)c(C(F)F)cc43)nn2C2CCN(C(=O)CN3CC(CNc4cccc5c4C(=O)N(C4CCC(=O)NC4=O)C5=O)C3)CC2)C1]

P-158 was synthesized following the standard procedure for preparing P-001 (77 mg, yield: 88%). MS (ESI) m/z: 892.9 [M+H]⁺.

### Example 244. 3-(4-((8-(4-(5-Acetyl-3-(7-(difluoromethyl)-6-(1-methyl-1H-pyrazol-4-yl)-3,4-dihydroquinolin-1(2H)-yl)-4,5,6,7-tetrahydro-1H-pyrazolo[4,3-c]pyridin-1-yl)piperidin-1-yl)-8-oxooctyl)amino)-3-methyl-2-oxo-2,3-dihydro-1H-benzo[d]imidazol-1-yl)piperidine-2,6-dione (P-159)

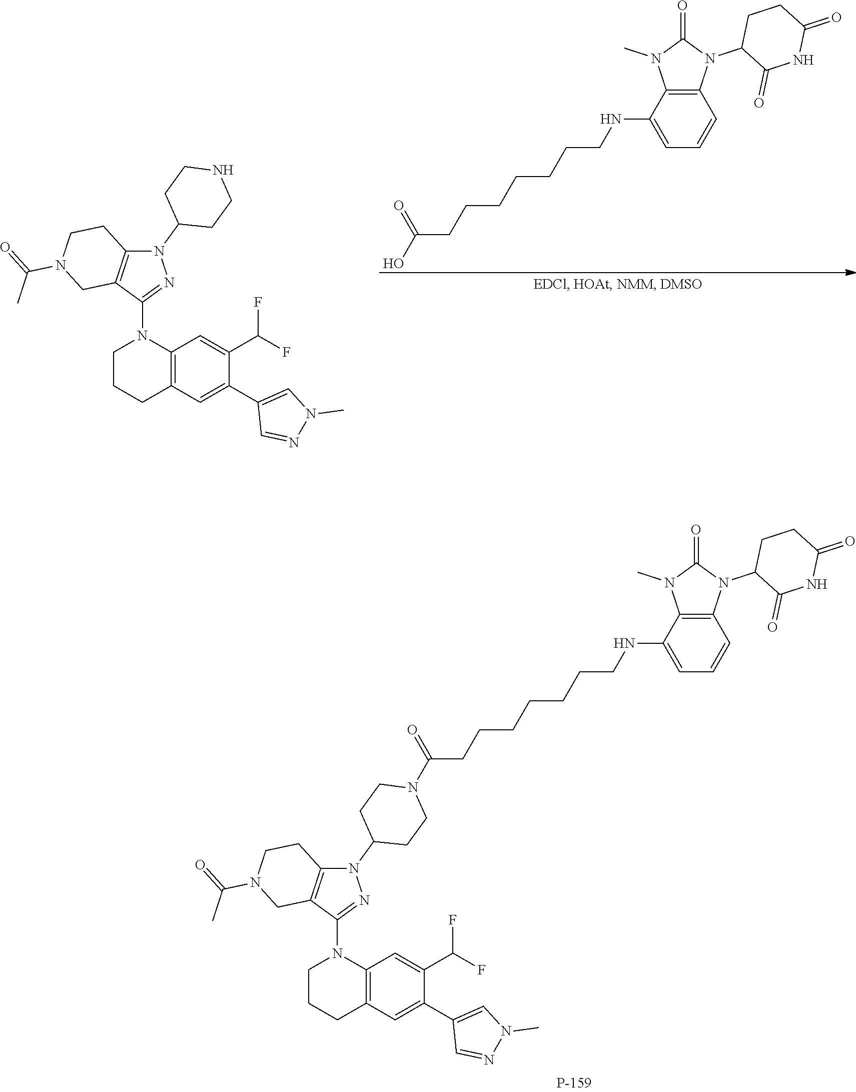 [SMILES: CC(=O)N1CCc2c(c(N3CCCc4cc(-c5cnn(C)c5)c(C(F)F)cc43)nn2C2CCNCC2)C1 ; Cn1c(=O)n(C2CCC(=O)NC2=O)c2cccc(NCCCCCCCC(=O)O)c21 ; CC(=O)N1CCc2c(c(N3CCCc4cc(-c5cnn(C)c5)c(C(F)F)cc43)nn2C2CCN(C(=O)CCCCCCCNc3cccc4c3n(C)c(=O)n4C3CCC(=O)NC3=O)CC2)C1]

P-159 was synthesized following the standard procedure for preparing P-109 (4.4 mg, yield: 40%). MS (ESI) m/z: 909.1 [M+H]⁺.

### Example 245. 3-(4-((9-(4-(5-Acetyl-3-(7-(difluoromethyl)-6-(1-methyl-1H-pyrazol-4-yl)-3,4-dihydroquinolin-1(2H)-yl)-4,5,6,7-tetrahydro-1H-pyrazolo[4,3-c]pyridin-1-yl)piperidin-1-yl)-9-oxononyl)amino)-3-methyl-2-oxo-2,3-dihydro-1H-benzo[d]imidazol-1-yl)piperidine-2,6-dione (P-160)

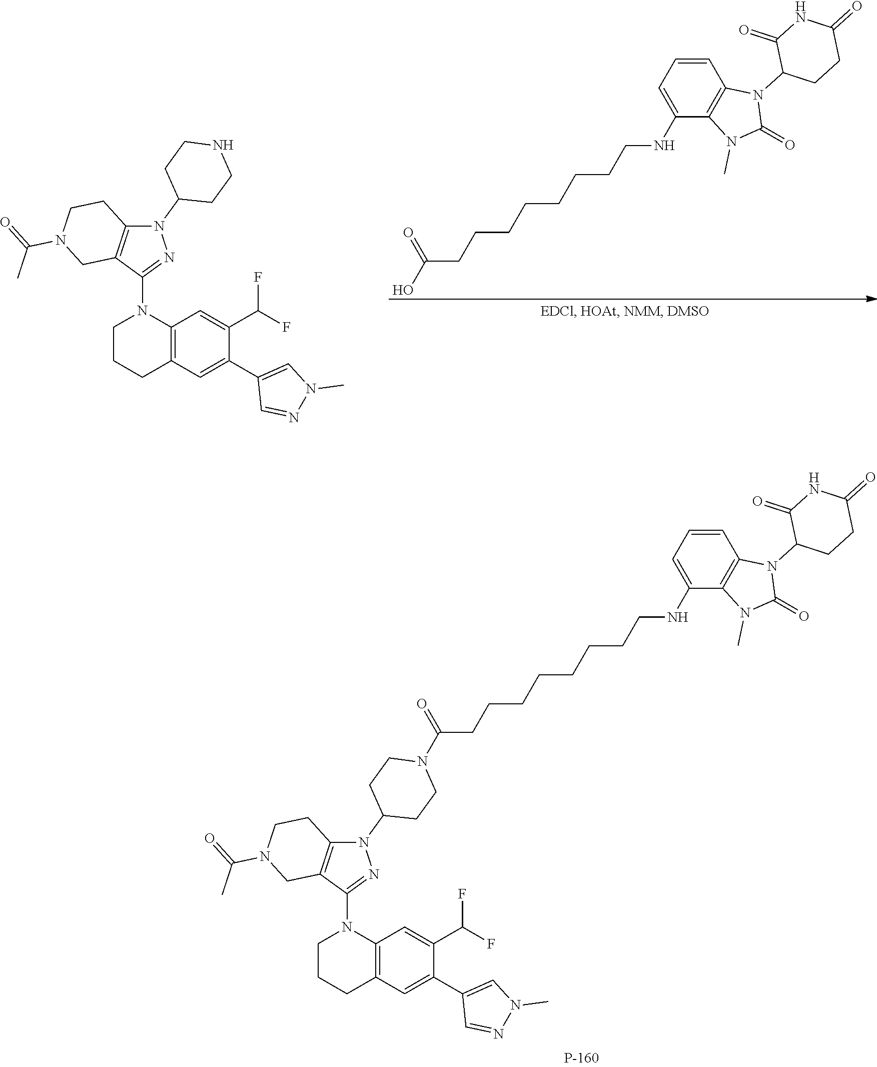 [SMILES: CC(=O)N1CCc2c(c(N3CCCc4cc(-c5cnn(C)c5)c(C(F)F)cc43)nn2C2CCNCC2)C1 ; Cn1c(=O)n(C2CCC(=O)NC2=O)c2cccc(NCCCCCCCCC(=O)O)c21 ; CC(=O)N1CCc2c(c(N3CCCc4cc(-c5cnn(C)c5)c(C(F)F)cc43)nn2C2CCN(C(=O)CCCCCCCCNc3cccc4c3n(C)c(=O)n4C3CCC(=O)NC3=O)CC2)C1]

P-160 was synthesized following the standard procedure for preparing P-109 (5.4 mg, yield: 36%). MS (ESI) m/z: 923.1 [M+H]⁺.

### Example 246. 3-(5-((8-(4-(5-Acetyl-3-(7-(difluoromethyl)-6-(1-methyl-1H-pyrazol-4-yl)-3,4-dihydroquinolin-1(2H)-yl)-4,5,6,7-tetrahydro-1H-pyrazolo[4,3-c]pyridin-1-yl)piperidin-1-yl)-8-oxooctyl)amino)-3-methyl-2-oxo-2,3-dihydro-1H-benzo[d]imidazol-1-yl)piperidine-2,6-dione (P-161)

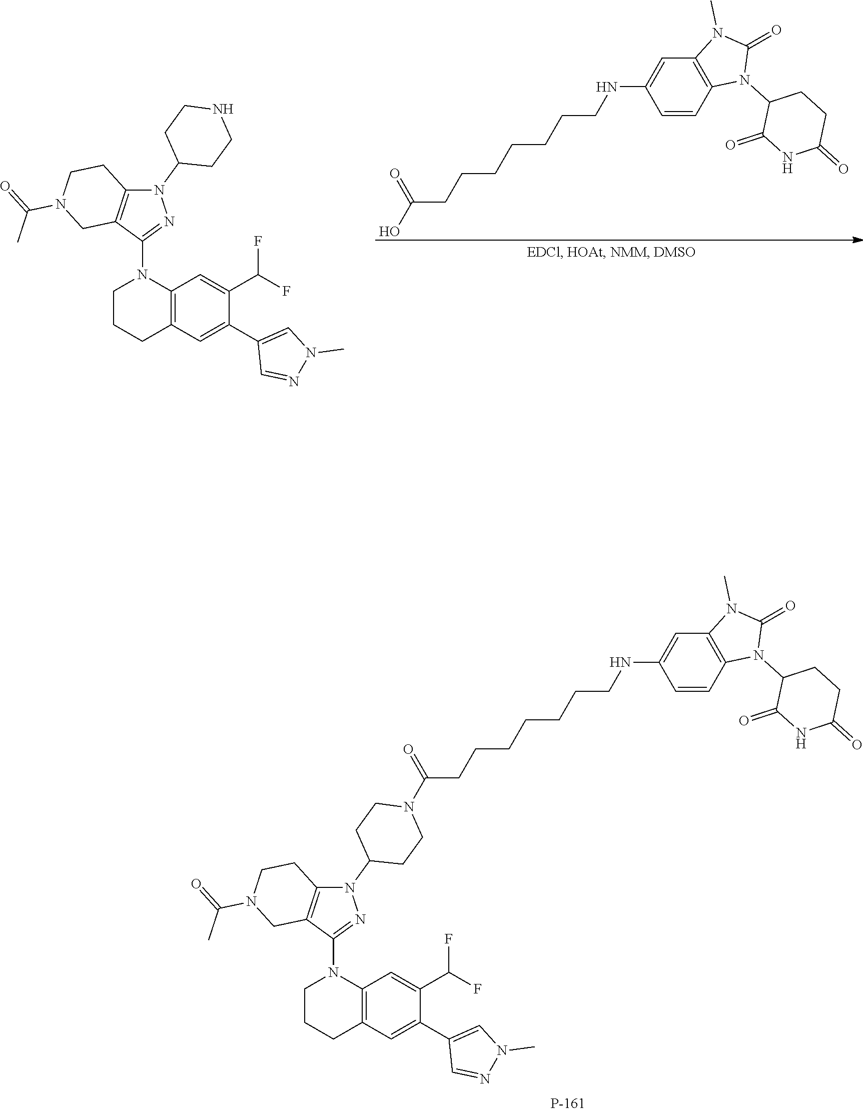 [SMILES: CC(=O)N1CCc2c(c(N3CCCc4cc(-c5cnn(C)c5)c(C(F)F)cc43)nn2C2CCNCC2)C1 ; Cn1c(=O)n(C2CCC(=O)NC2=O)c2ccc(NCCCCCCCC(=O)O)cc21 ; CC(=O)N1CCc2c(c(N3CCCc4cc(-c5cnn(C)c5)c(C(F)F)cc43)nn2C2CCN(C(=O)CCCCCCCNc3ccc4c(c3)n(C)c(=O)n4C3CCC(=O)NC3=O)CC2)C1]

P-161 was synthesized following the standard procedure for preparing P-109 (3.5 mg, yield: 32%). MS (ESI) m/z: 909.0 [M+H]⁺.

### Example 247. 3-(5-((9-(4-(5-Acetyl-3-(7-(difluoromethyl)-6-(1-methyl-1H-pyrazol-4-yl)-3,4-dihydroquinolin-1(2H)-yl)-4,5,6,7-tetrahydro-1H-pyrazolo[4,3-c]pyridin-1-yl)piperidin-1-yl)-9-oxononyl)amino)-3-methyl-2-oxo-2,3-dihydro-1H-benzo[d]imidazol-1-yl)piperidine-2,6-dione (P-162)

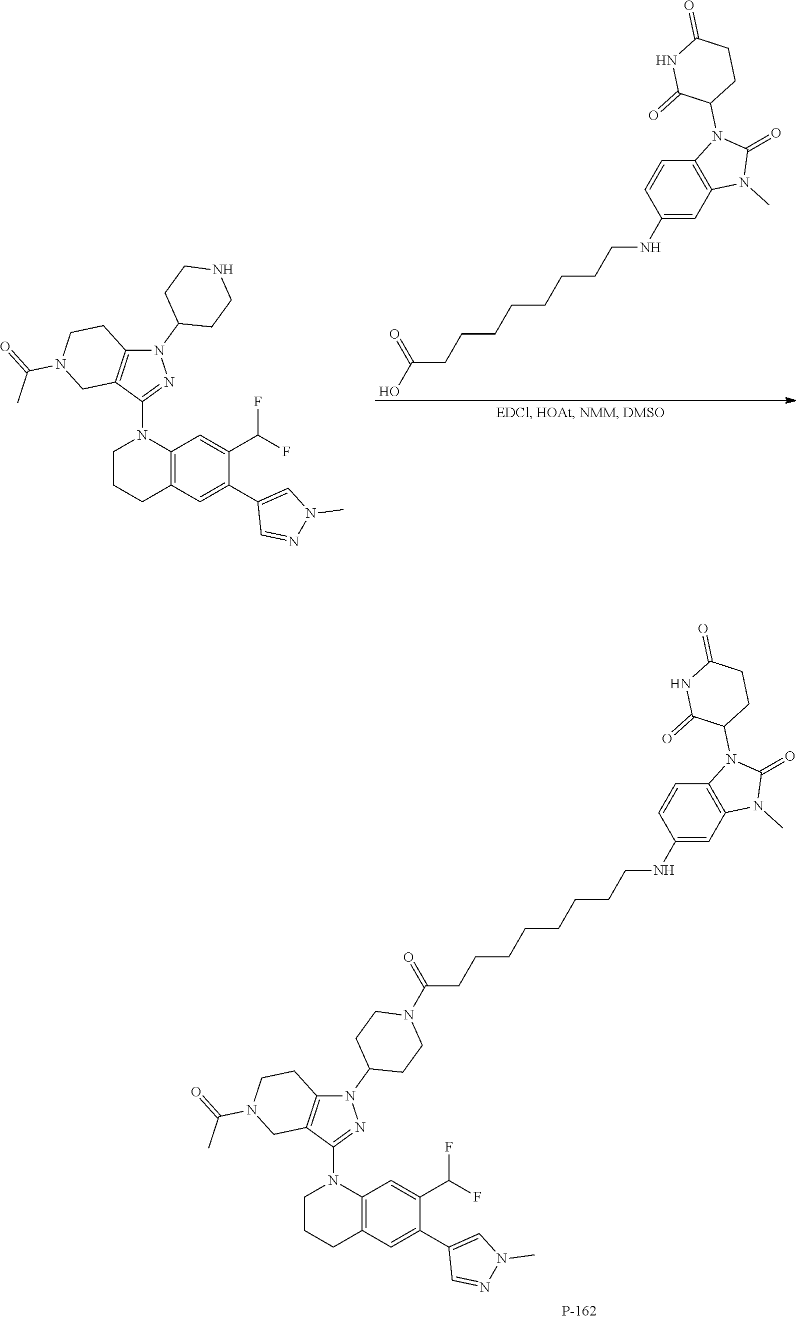 [SMILES: CC(=O)N1CCc2c(c(N3CCCc4cc(-c5cnn(C)c5)c(C(F)F)cc43)nn2C2CCNCC2)C1 ; Cn1c(=O)n(C2CCC(=O)NC2=O)c2ccc(NCCCCCCCCC(=O)O)cc21 ; CC(=O)N1CCc2c(c(N3CCCc4cc(-c5cnn(C)c5)c(C(F)F)cc43)nn2C2CCN(C(=O)CCCCCCCCNc3ccc4c(c3)n(C)c(=O)n4C3CCC(=O)NC3=O)CC2)C1]

P-162 was synthesized following the standard procedure for preparing P-109 (4.3 mg, yield: 40%). MS (ESI) m/z: 923.0 [M+H]⁺.

### Example 248. 3-(3-(9-(4-(5-Acetyl-3-(7-(difluoromethyl)-6-(1-methyl-1H-pyrazol-4-yl)-3,4-dihydroquinolin-1(2H)-yl)-4,5,6,7-tetrahydro-1H-pyrazolo[4,3-c]pyridin-1-yl)piperidin-1-yl)-9-oxononyl)-2-oxo-2,3-dihydro-1H-benzo[d]imidazol-1-yl)piperidine-2,6-dione (P-163)

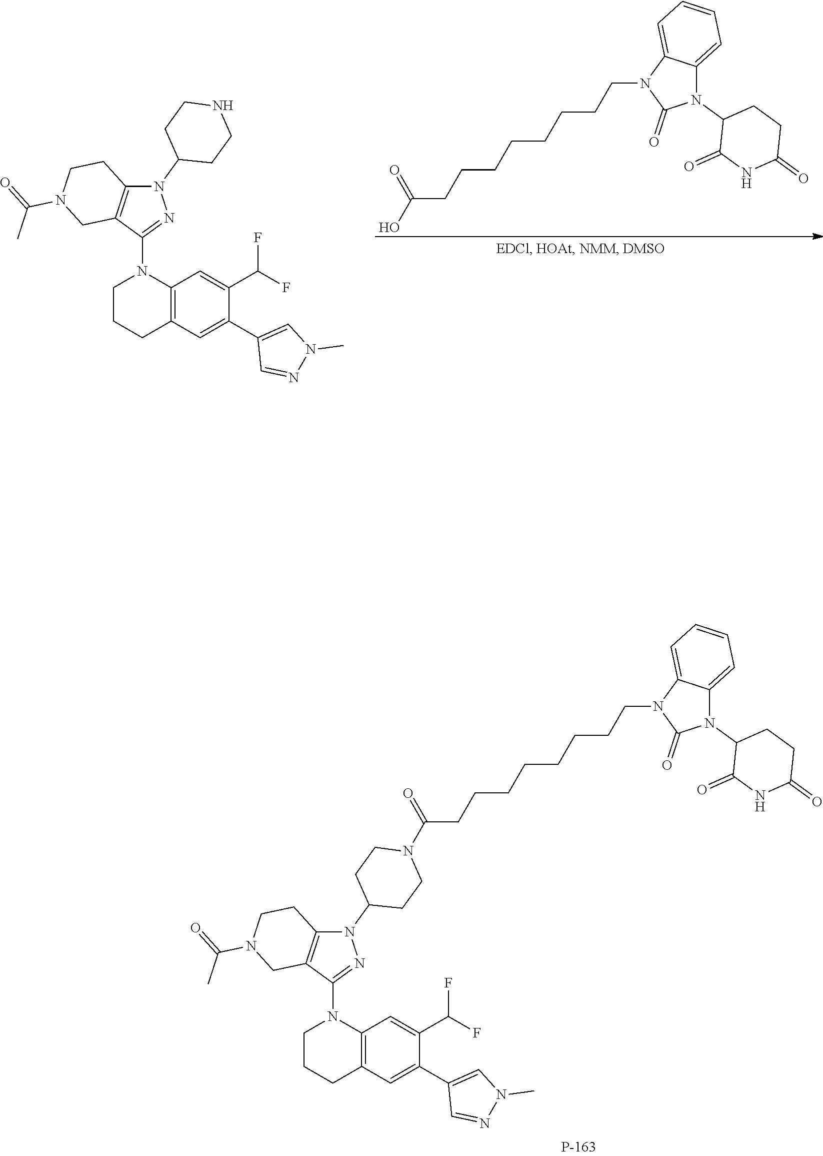 [SMILES: CC(=O)N1CCc2c(c(N3CCCc4cc(-c5cnn(C)c5)c(C(F)F)cc43)nn2C2CCNCC2)C1 ; O=C(O)CCCCCCCCn1c(=O)n(C2CCC(=O)NC2=O)c2ccccc21 ; CC(=O)N1CCc2c(c(N3CCCc4cc(-c5cnn(C)c5)c(C(F)F)cc43)nn2C2CCN(C(=O)CCCCCCCCn3c(=O)n(C4CCC(=O)NC4=O)c4ccccc43)CC2)C1]

P-163 was synthesized following the standard procedure for preparing P-105 (48 mg, yield: 72%). MS (ESI) m/z: 894.0 [M+H]⁺.

### Example 249. 3-(3-(10-(4-(5-Acetyl-3-(7-(difluoromethyl)-6-(1-methyl-1H-pyrazol-4-yl)-3,4-dihydroquinolin-1(2H)-yl)-4,5,6,7-tetrahydro-1H-pyrazolo[4,3-c]pyridin-1-yl)piperidin-1-yl)-10-oxodecyl)-2-oxo-2,3-dihydro-1H-benzo[d] imidazol-1-yl)piperidine-2,6-dione (P-164)

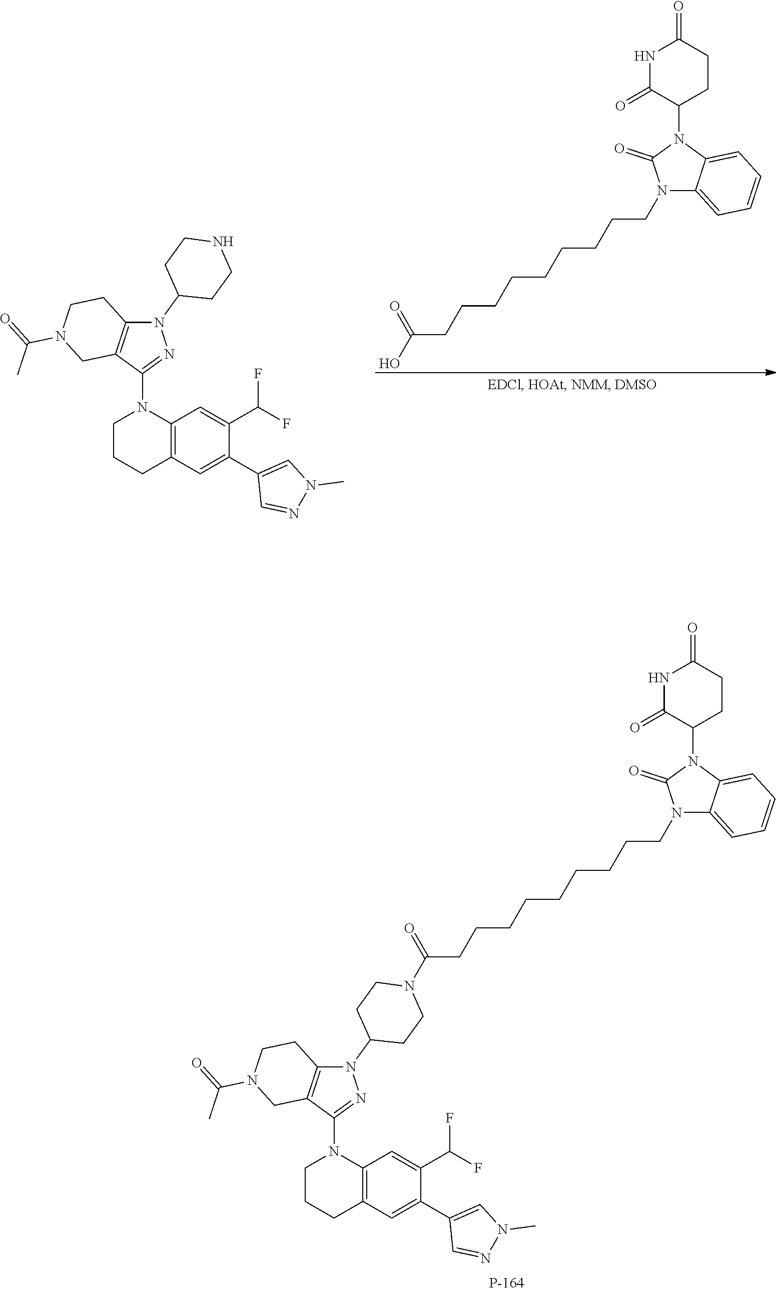 [SMILES: CC(=O)N1CCc2c(c(N3CCCc4cc(-c5cnn(C)c5)c(C(F)F)cc43)nn2C2CCNCC2)C1 ; O=C(O)CCCCCCCCCn1c(=O)n(C2CCC(=O)NC2=O)c2ccccc21 ; CC(=O)N1CCc2c(c(N3CCCc4cc(-c5cnn(C)c5)c(C(F)F)cc43)nn2C2CCN(C(=O)CCCCCCCCCn3c(=O)n(C4CCC(=O)NC4=O)c4ccccc43)CC2)C1]

P-164 was synthesized following the standard procedure for preparing P-105 (37 mg, yield: 56%). MS (ESI) m/z: 908.0 [M+H]⁺.

### Example 250. 3-(5-((6-(4-(5-Acetyl-3-(7-(difluoromethyl)-6-(1-methyl-1H-pyrazol-4-yl)-3,4-dihydroquinolin-1(2H)-yl)-4,5,6,7-tetrahydro-1H-pyrazolo[4,3-c]pyridin-1-yl)piperidin-1-yl)-6-oxohexyl)amino)-2-methyl-4-oxoquinazolin-3(4H)-yl)piperidine-2,6-dione (P-165)

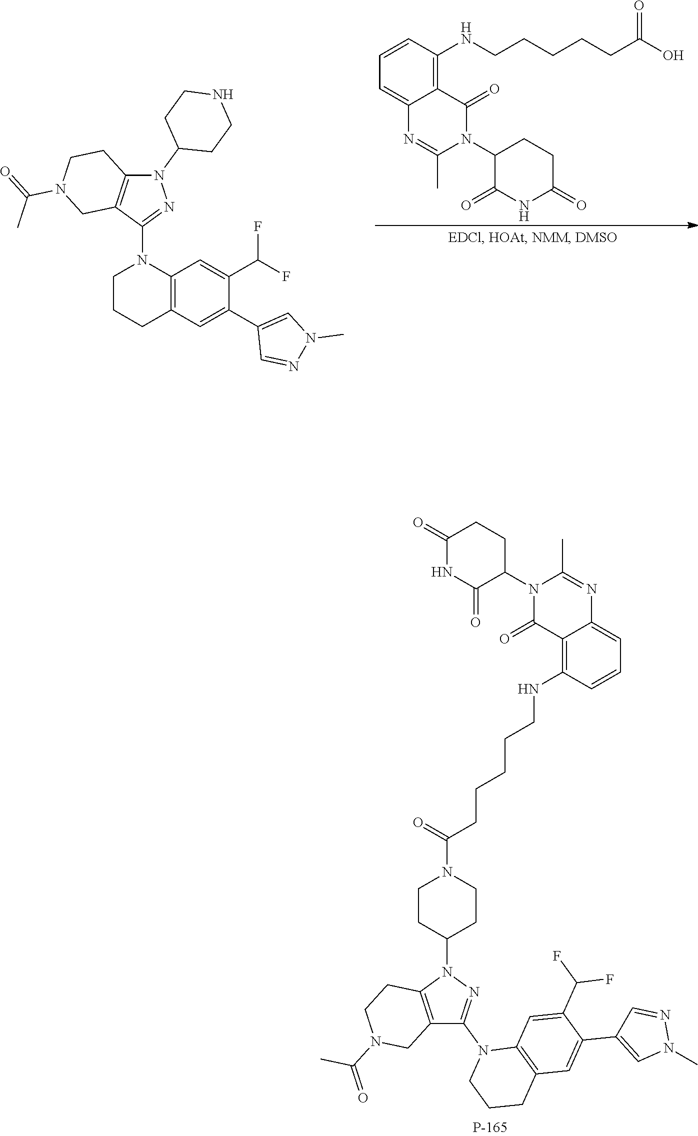 [SMILES: CC(=O)N1CCc2c(c(N3CCCc4cc(-c5cnn(C)c5)c(C(F)F)cc43)nn2C2CCNCC2)C1 ; Cc1nc2cccc(NCCCCCC(=O)O)c2c(=O)n1C1CCC(=O)NC1=O ; CC(=O)N1CCc2c(c(N3CCCc4cc(-c5cnn(C)c5)c(C(F)F)cc43)nn2C2CCN(C(=O)CCCCCNc3cccc4nc(C)n(C5CCC(=O)NC5=O)c(=O)c34)CC2)C1]

P-165 was synthesized following the standard procedure for preparing P-151 (6.2 mg, yield: 35%). MS (ESI) ink: 892.9 [M+H]⁺.

### Example 251. 3-(4-((4-((4-(5-Acetyl-3-(7-(difluoromethyl)-6-(1-methyl-1H-pyrazol-4-yl)-3,4-dihydroquinolin-1(2H)-yl)-4,5,6,7-tetrahydro-1H-pyrazolo[4,3-c]pyridin-1-yl)piperidin-1-yl)methyl)benzyl)oxy)-1-oxoisoindolin-2-yl)piperidine-2,6-dione (P-166)

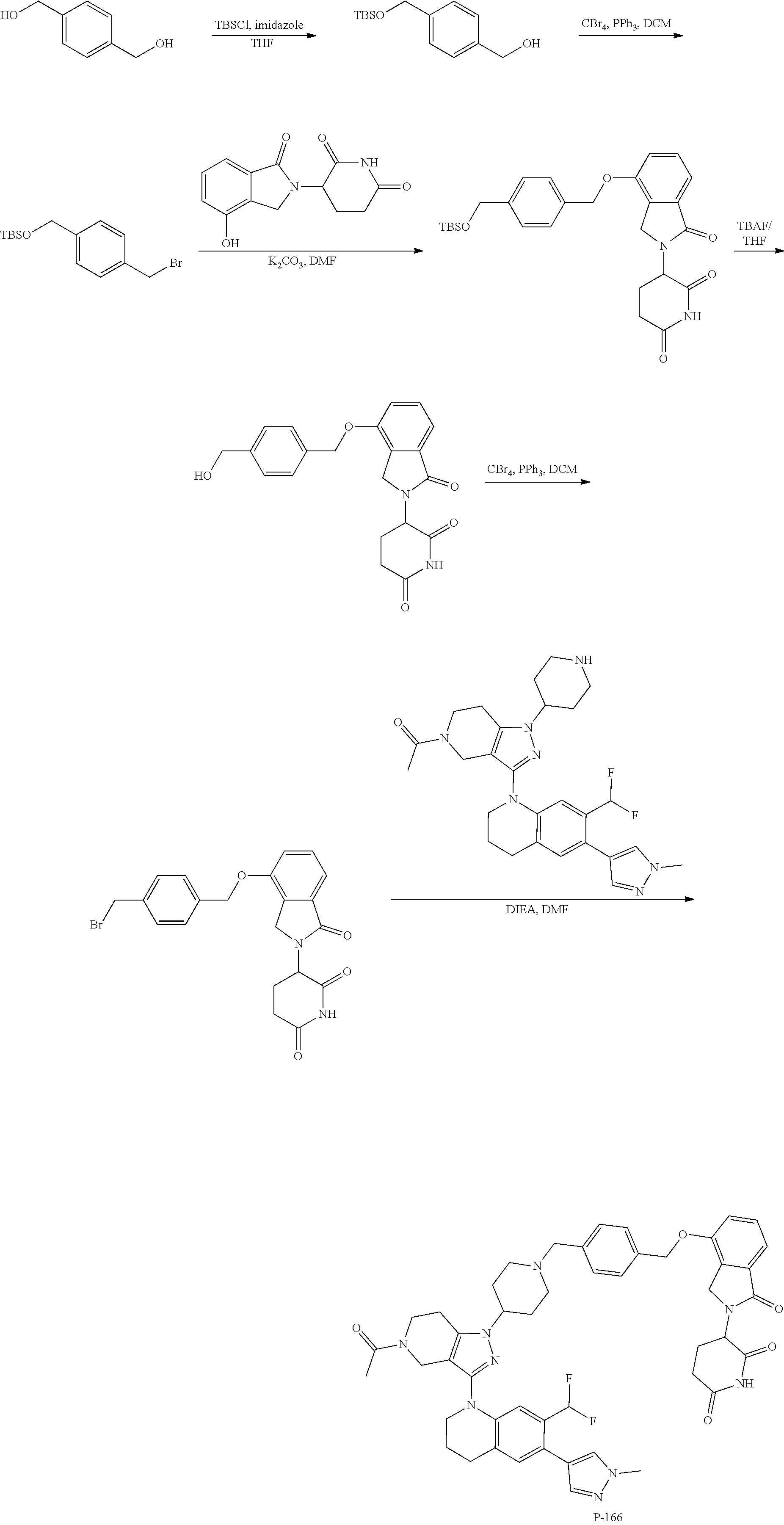

### Step 1. Synthesis of (4-(((tert-butyldimethylsilyl)oxy)methyl)phenyl)methanol

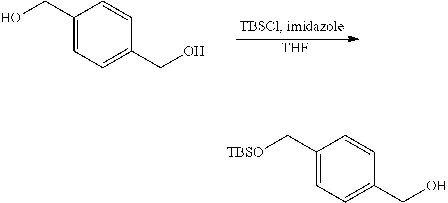 [SMILES: OCc1ccc(CO)cc1 ; CC(C)(C)[Si](C)(C)OCc1ccc(CO)cc1 ; "THF"]

To a solution of 1,4-phenylenedimethanol (13.8 g, 99.88 mmol) in DCM (400 mL) was added imidazole (13.6 g, 199.76 mmol) followed by TBSCl (15.05 g, 99.88 mmol) at 0° C. The resulting mixture was stirred at room temperature for 16 h, before it was concentrated. The resulting residue was purified by silica gel chromatography (petroleum ether:EtOAc=10:1 to 5:1) to give the desired product (15 g, yield: 59%) as colorless oil.

### Step 2. Synthesis of ((4-(bromomethyl)benzyl)oxy)(tert-butyl)dimethylsilane

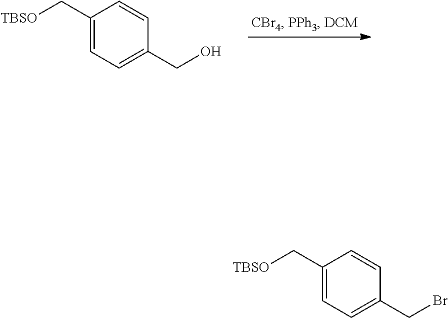 [SMILES: CC(C)(C)[Si](C)(C)OCc1ccc(CO)cc1 ; CC(C)(C)[Si](C)(C)OCc1ccc(CBr)cc1]

To a solution of (4-(((tert-butyldimethylsilyl)oxy)methyl)phenyl)methanol (252 mg, 1 mmol) in DCM (10 mL) were added CBr₄ (496 mg, 1.5 mmol) and PPh₃ (392 mg, 1.5 mmol) at 0° C. The resulting mixture was stirred at room temperature for 4 h, before it was concentrated. The resulting residue was purified by prep-TLC to give the desired product (240 mg, yield: 76%) as white solid.

### Step 3. Synthesis of 3-(4-((4-(hydroxymethyl)benzyl)oxy)-1-oxoisoindolin-2-yl)piperidine-2,6-dione

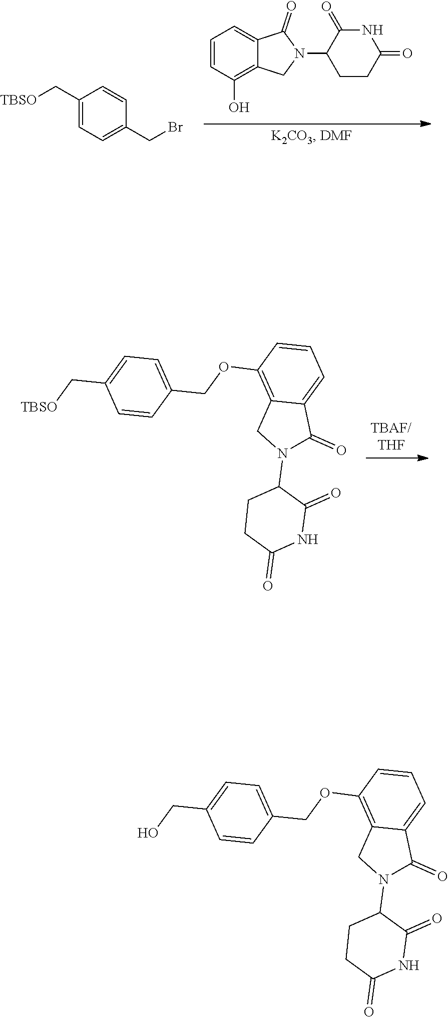 [SMILES: CC(C)(C)[Si](C)(C)OCc1ccc(CBr)cc1 ; O=C1CCC(N2Cc3c(O)cccc3C2=O)C(=O)N1 ; CC(C)(C)[Si](C)(C)OCc1ccc(COc2cccc3c2CN(C2CCC(=O)NC2=O)C3=O)cc1 ; O=C1CCC(N2Cc3c(OCc4ccc(CO)cc4)cccc3C2=O)C(=O)N1]

To a solution of ((4-(bromomethyl)benzyl)oxy)(tert-butyl)dimethylsilane (770 mg, 2.44 mmol) and 3-(4-hydroxy-1-oxoisoindolin-2-yl)piperidine-2,6-dione (635 mg, 2.44 mmol) in DMF (10 mL) was added K₂CO₃ (674 mg, 4.88 mmol). The resulting mixture was stirred at 40° C. for 16 h, before TBAF (2.55 g, 9.77 mmol) was added. The reaction mixture was purified by prep-HPLC to give the desired product (450 mg, yield: 48%) as white solid. MS (ESI) m/z: 381.3 [M+H]⁺.

### Step 4. Synthesis of 3-(4-((4-(bromomethyl)benzyl)oxy)-1-oxoisoindolin-2-yl)piperidine-2,6-dione

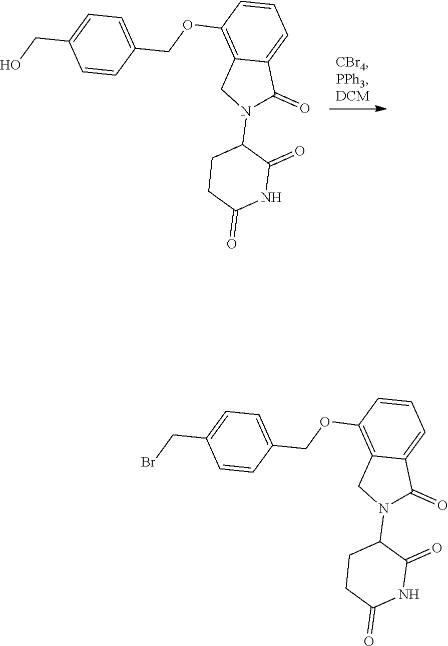 [SMILES: O=C1CCC(N2Cc3c(OCc4ccc(CO)cc4)cccc3C2=O)C(=O)N1 ; O=C1CCC(N2Cc3c(OCc4ccc(CBr)cc4)cccc3C2=O)C(=O)N1]

To a solution of 3-(4-((4-(hydroxymethyl)benzyl)oxy)-1-oxoisoindolin-2-yl)piperidine-2,6-dione (100 mg, 0.26 mmol) in DCM (10 mL) were added CBr₄ (105 mg, 0.32 mmol) and PPh₃ (103 mg, 0.39 mmol) at 0° C. The resulting mixture was stirred at room temperature for 2 h, before it was concentrated. The resulting residue was purified by silica gel chromatography (DCM/MeOH=30:1 to 15:1) to give the desired product (90 mg, yield: 77%) as white solid. MS (ESI) m/z: 443.1 [M+H]⁺.

### Step 5. Synthesis of 3-(4-((4-((4-(5-acetyl-3-(7-(difluoromethyl)-6-(1-methyl-1H-pyrazol-4-yl)-3,4-dihydroquinolin-1(2H)-yl)-4,5,6,7-tetrahydro-1H-pyrazolo[4,3-c]pyridin-1-yl)piperidin-1-yl)methyl)benzyl)oxy)-1-oxoisoindolin-2-yl)piperidine-2,6-dione

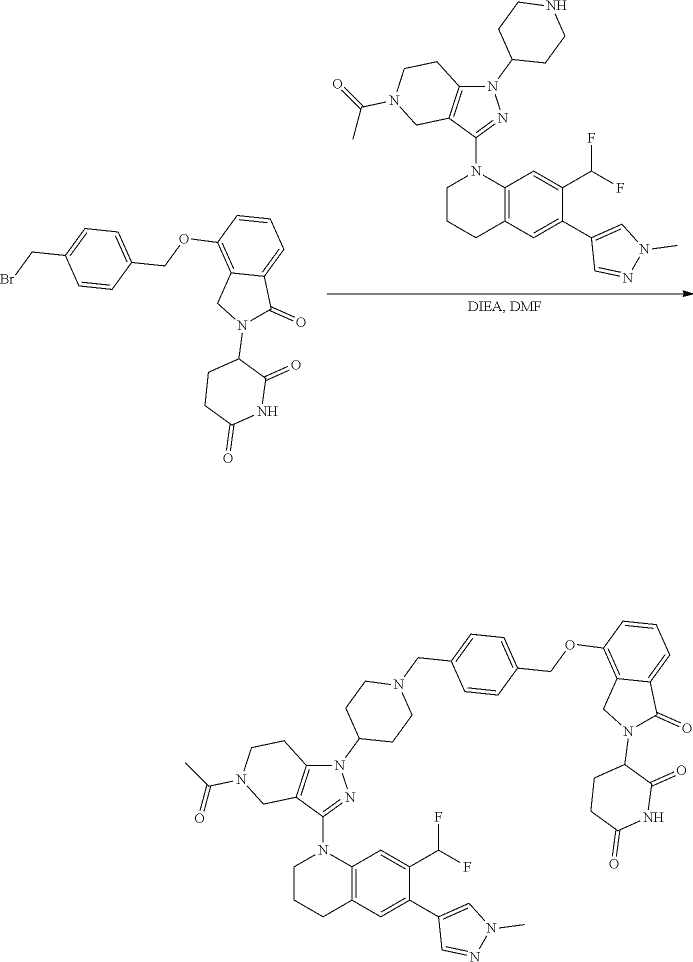 [SMILES: O=C1CCC(N2Cc3c(OCc4ccc(CBr)cc4)cccc3C2=O)C(=O)N1 ; CC(=O)N1CCc2c(c(N3CCCc4cc(-c5cnn(C)c5)c(C(F)F)cc43)nn2C2CCNCC2)C1 ; CC(=O)N1CCc2c(c(N3CCCc4cc(-c5cnn(C)c5)c(C(F)F)cc43)nn2C2CCN(Cc3ccc(COc4cccc5c4CN(C4CCC(=O)NC4=O)C5=O)cc3)CC2)C1]

P-166 was synthesized following the standard procedure for preparing P-143 (50 mg, yield: 63%). MS (ESI) m/z: 873.1 [M+H]⁺.

### Example 252. 4-((1-(2-(4-(5-Acetyl-3-(7-(difluoromethyl)-6-(1-methyl-1H-pyrazol-4-yl)-3,4-dihydroquinolin-1(2H)-yl)-4,5,6,7-tetrahydro-1H-pyrazolo[4,3-c]pyridin-1-yl)piperidin-1-yl)ethyl)piperidin-4-yl)methoxy)-2-(2,6-dioxopiperidin-3-yl)isoindoline-1,3-dione (P-167)

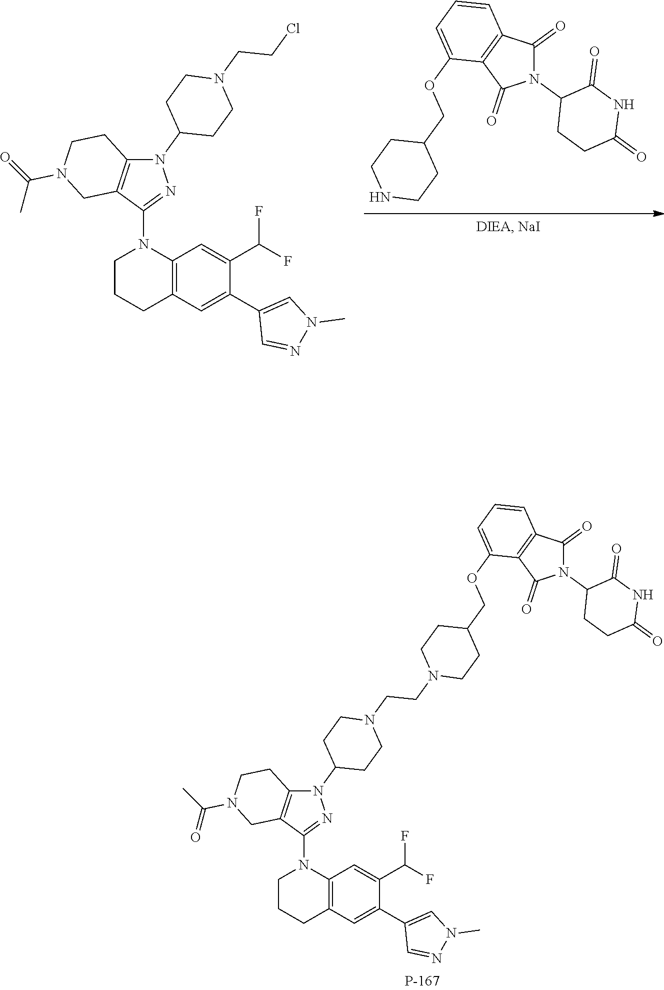 [SMILES: CC(=O)N1CCc2c(c(N3CCCc4cc(-c5cnn(C)c5)c(C(F)F)cc43)nn2C2CCN(CCCl)CC2)C1 ; O=C1CCC(N2C(=O)c3cccc(OCC4CCNCC4)c3C2=O)C(=O)N1 ; CC(=O)N1CCc2c(c(N3CCCc4cc(-c5cnn(C)c5)c(C(F)F)cc43)nn2C2CCN(CCN3CCC(COc4cccc5c4C(=O)N(C4CCC(=O)NC4=O)C5=O)CC3)CC2)C1]

P-167 was synthesized following the standard procedure for preparing P-157 (4 mg, yield: 9%). MS (ESI) m/z: 907.0 [M+H]⁺.

### Example 253. 4-((1-(2-(4-(5-Acetyl-3-(7-(difluoromethyl)-6-(1-methyl-1H-pyrazol-4-yl)-3,4-dihydroquinolin-1(2H)-yl)-4,5,6,7-tetrahydro-1H-pyrazolo[4,3-c]pyridin-1-yl)piperidin-1-yl)-2-oxoethyl)piperidin-4-yl)methoxy)-2-(2,6-dioxopiperidin-3-yl)isoindoline-1,3-dione (P-168)

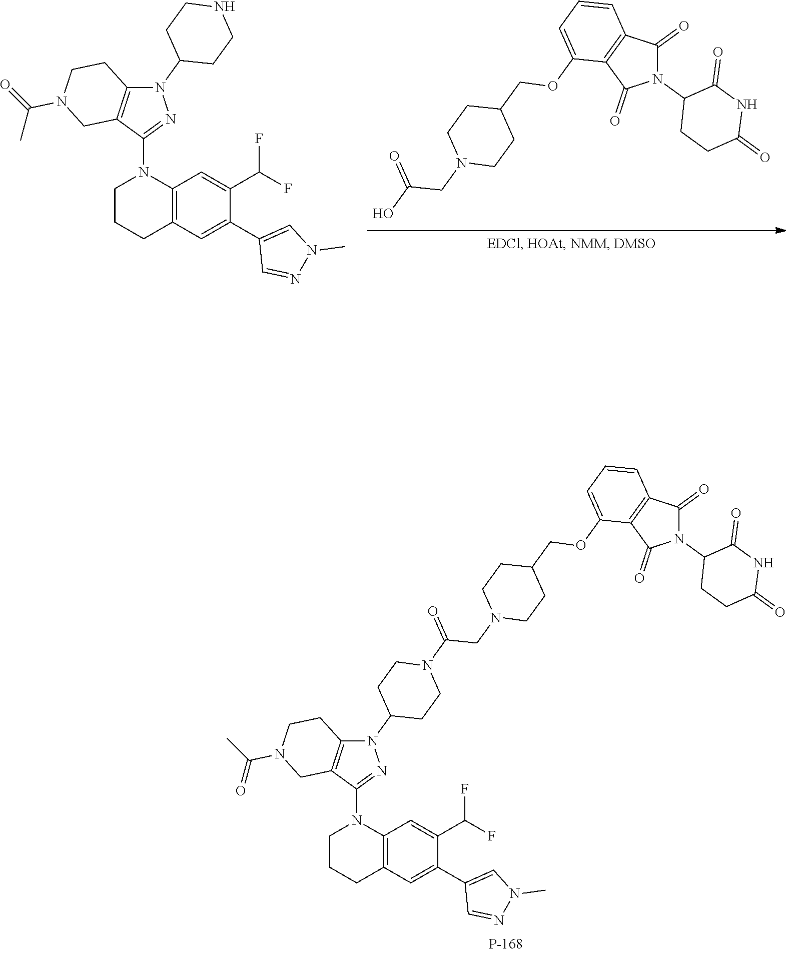

P-168 was synthesized following the standard procedure for preparing P-001 (16.5 mg, yield: 77%). MS (ESI) m/z: 921.9 [M+H]⁺.

### Example 254. 4-(2-(4-(2-(4-(5-Acetyl-3-(7-(difluoromethyl)-6-(1-methyl-1H-pyrazol-4-yl)-3,4-dihydroquinolin-1(2H)-yl)-4,5,6,7-tetrahydro-1H-pyrazolo[4,3-c]pyridin-1-yl)piperidin-1-yl)-2-oxoethyl)piperazin-1-yl)ethyl)-2-(2,6-dioxopiperidin-3-yl)isoindoline-1,3-dione (P-169)

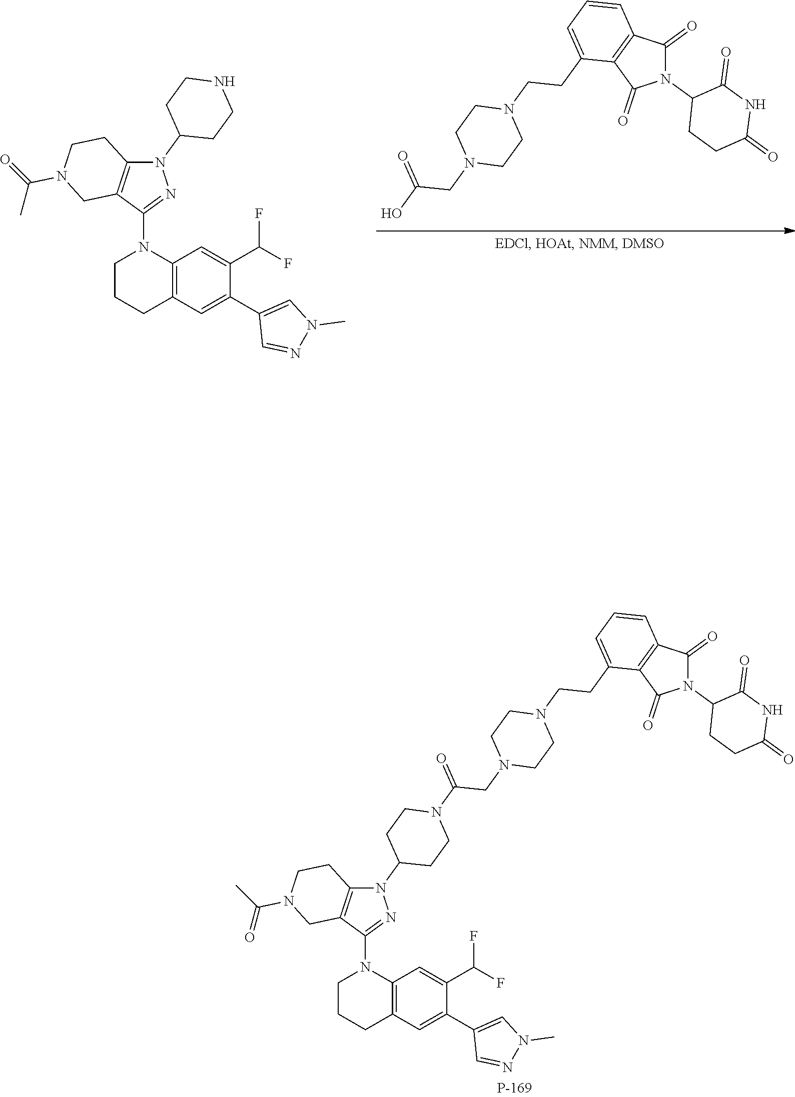 [SMILES: CC(=O)N1CCc2c(c(N3CCCc4cc(-c5cnn(C)c5)c(C(F)F)cc43)nn2C2CCNCC2)C1 ; O=C(O)CN1CCN(CCc2cccc3c2C(=O)N(C2CCC(=O)NC2=O)C3=O)CC1 ; CC(=O)N1CCc2c(c(N3CCCc4cc(-c5cnn(C)c5)c(C(F)F)cc43)nn2C2CCN(C(=O)CN3CCN(CCc4cccc5c4C(=O)N(C4CCC(=O)NC4=O)C5=O)CC3)CC2)C1]

P-169 was synthesized following the standard procedure for preparing P-001 (32 mg, yield: 75%). MS (ESI) m/z: 921.0 [M+H]⁺.

### Example 255. 4-((2-(1-(2-(4-(5-Acetyl-3-(7-(difluoromethyl)-6-(1-methyl-1H-pyrazol-4-yl)-3,4-dihydroquinolin-1(2H)-yl)-4,5,6,7-tetrahydro-1H-pyrazolo[4,3-c]pyridin-1-yl)piperidin-1-yl)-2-oxoethyl)azetidin-3-yl)ethyl)amino)-2-(2,6-dioxopiperidin-3-yl)isoindoline-1,3-dione (P-170)

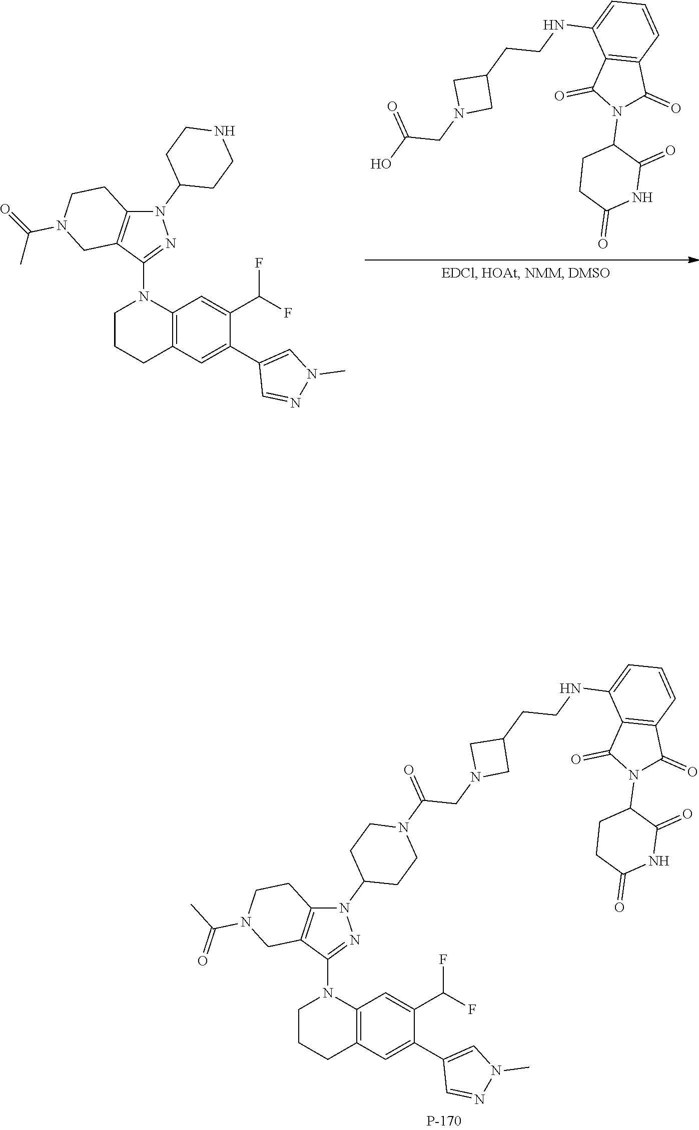

P-170 was synthesized following the standard procedure for preparing P-001 (2.1 mg, yield: 19%). MS (ESI) m/z: 906.8 [M+H]⁺.

### Example 256. 4-((6-(4-(5-Acetyl-3-(7-(difluoromethyl)-6-(1-methyl-1H-pyrazol-4-yl)-3,4-dihydroquinolin-1(2H)-yl)-4,5,6,7-tetrahydro-1H-pyrazolo[4,3-c]pyridin-1-yl)piperidin-1-yl)-6-oxohex-3-yn-1-yl)amino)-2-(2,6-dioxopiperidin-3-yl)isoindoline-1,3-dione (P-171)

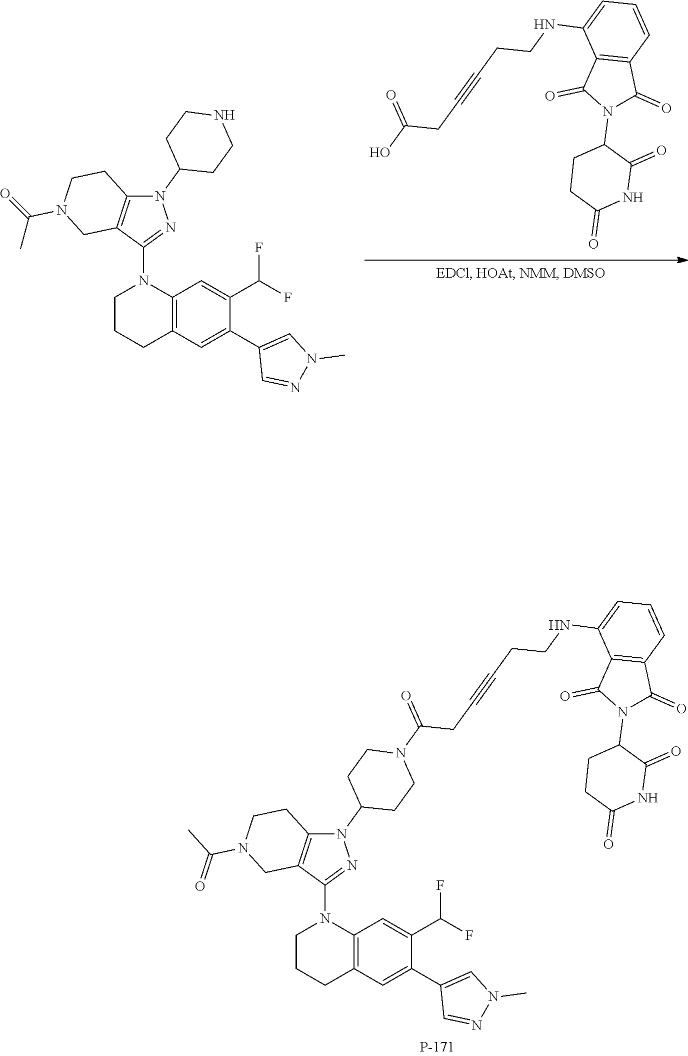 [SMILES: CC(=O)N1CCc2c(c(N3CCCc4cc(-c5cnn(C)c5)c(C(F)F)cc43)nn2C2CCNCC2)C1 ; O=C(O)CC#CCCNc1cccc2c1C(=O)N(C1CCC(=O)NC1=O)C2=O ; CC(=O)N1CCc2c(c(N3CCCc4cc(-c5cnn(C)c5)c(C(F)F)cc43)nn2C2CCN(C(=O)CC#CCCNc3cccc4c3C(=O)N(C3CCC(=O)NC3=O)C4=O)CC2)C1]

P-171 was synthesized following the standard procedure for preparing P-001 (6.4 mg, yield: 56%). MS (ESI) m/z: 875.9 [M+H]⁺.

### Example 257. 4-((2-(4-(2-(4-(5-Acetyl-3-(7-(difluoromethyl)-6-(1-methyl-1H-pyrazol-4-yl)-3,4-dihydroquinolin-1(2H)-yl)-4,5,6,7-tetrahydro-1H-pyrazolo[4,3-c]pyridin-1-yl)piperidin-1-yl)-2-oxoethyl)morpholin-2-yl)ethyl)amino)-2-(2,6-dioxopiperidin-3-yl)isoindoline-1,3-dione (P-172)

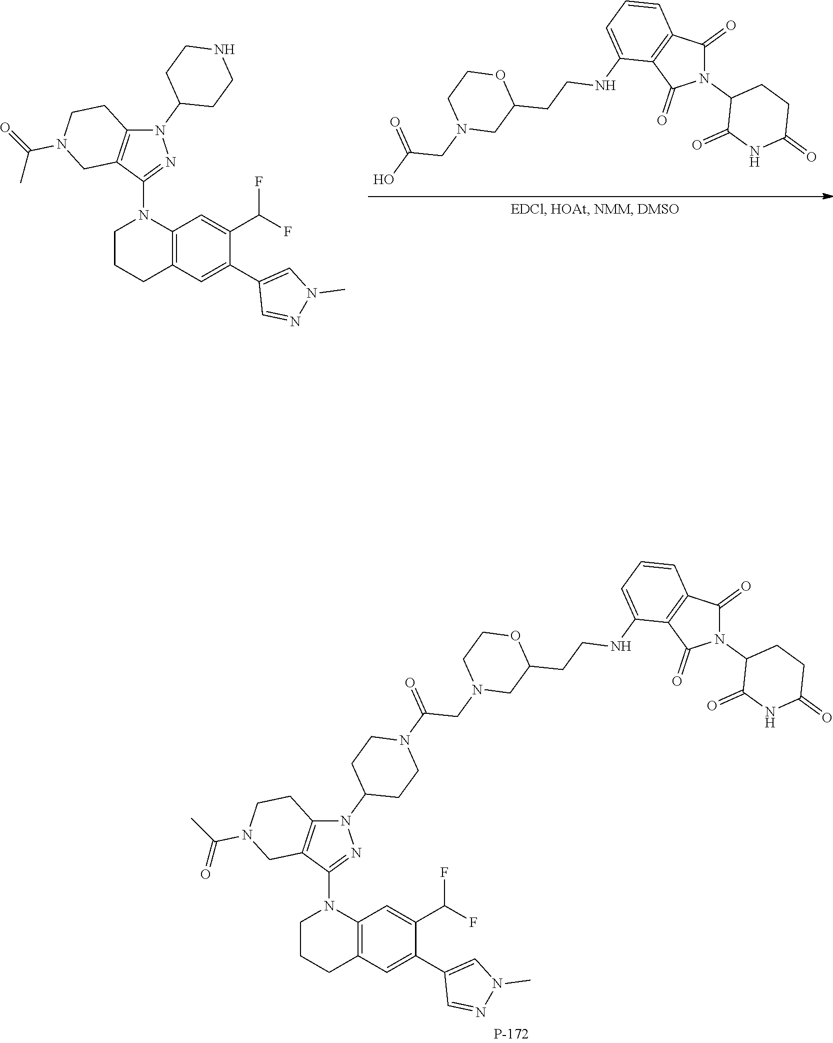

P-172 was synthesized following the standard procedure for preparing P-001 (16 mg, yield: 76%). MS (ESI) m/z: 936.9 [M+H]⁺.

### Example 258. 3-(4-((4-(2-(4-(5-Acetyl-3-(7-(difluoromethyl)-6-(1-methyl-1H-pyrazol-4-yl)-3,4-dihydroquinolin-1(2H)-yl)-4,5,6,7-tetrahydro-1H-pyrazolo[4,3-c]pyridin-1-yl)piperidin-1-yl)ethyl)benzyl)oxy)-1-oxoisoindolin-2-yl)piperidine-2,6-dione (P-173)

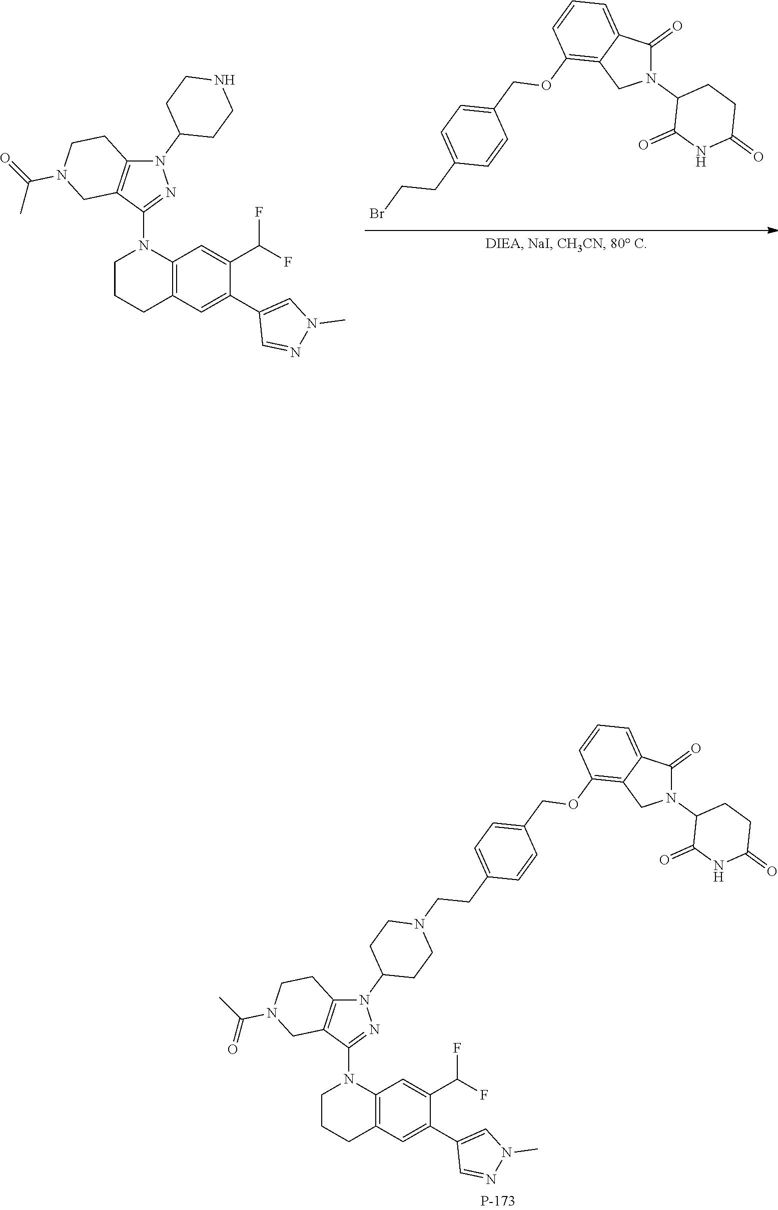 [SMILES: CC(=O)N1CCc2c(c(N3CCCc4cc(-c5cnn(C)c5)c(C(F)F)cc43)nn2C2CCNCC2)C1 ; O=C1CCC(N2Cc3c(OCc4ccc(CCBr)cc4)cccc3C2=O)C(=O)N1 ; CC(=O)N1CCc2c(c(N3CCCc4cc(-c5cnn(C)c5)c(C(F)F)cc43)nn2C2CCN(CCc3ccc(COc4cccc5c4CN(C4CCC(=O)NC4=O)C5=O)cc3)CC2)C1]

P-173 was synthesized following the standard procedure for preparing P-166 (41 mg, yield: 42%). MS (ESI) m/z: 886.8 [M+H]⁺.

### Example 259. 3-(4-(4-((4-(5-Acetyl-3-(7-(difluoromethyl)-6-(1-methyl-1H-pyrazol-4-yl)-3,4-dihydroquinolin-1(2H)-yl)-4,5,6,7-tetrahydro-1H-pyrazolo[4,3-c]pyridin-1-yl)piperidin-1-yl)methyl)phenethoxy)-1-oxoisoindolin-2-yl)piperidine-2,6-dione (P-174)

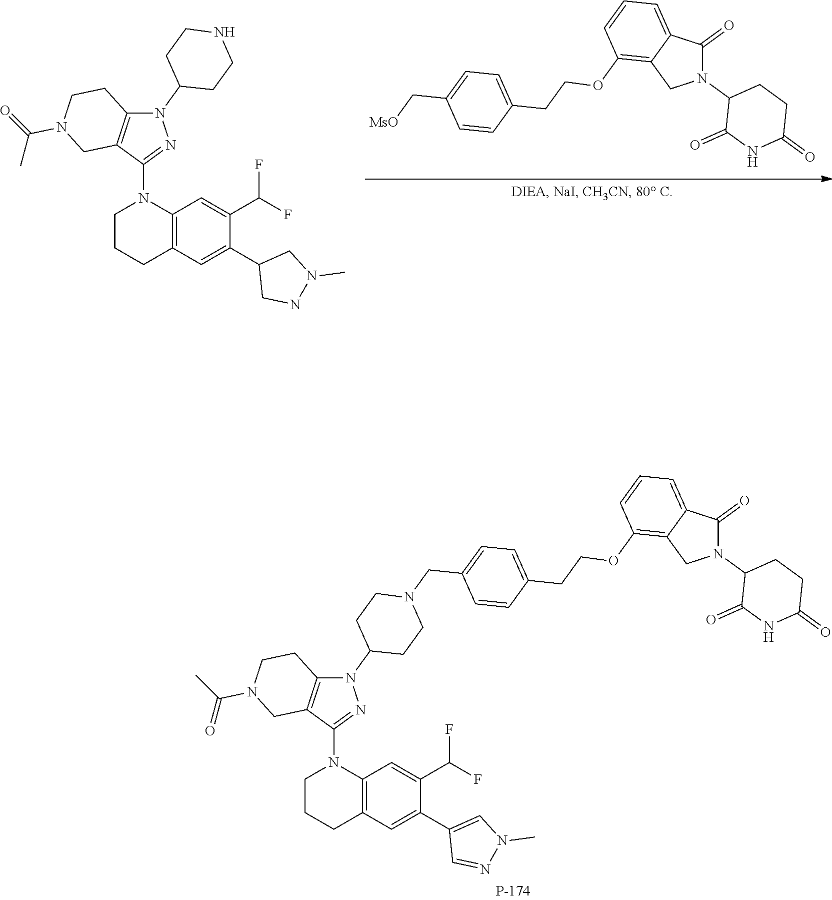 [1367] P-174 was synthesized following the standard procedure for preparing P-166 (4.2 mg, yield: 37%). MS (ESI) m/z: 887.1 [M+H]⁺.

Certain compounds disclosed herein have the structures shown in Table 1.

TABLE 1

| Cpd. Code | Structure | Chemical Name |
|---|---|---|
| P-001 | 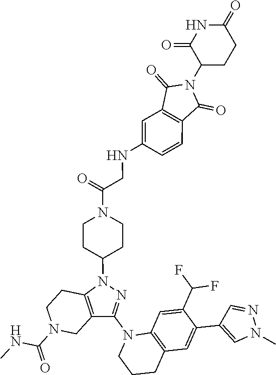 | 3-(7-(difluoromethyl)-6-(1-methyl- 1H-pyrazol-4-yl)-3,4- dihydroquinolin-1(2H)-yl)-1-(1-((2- (2,6-dioxopiperidin-3-yl)-1,3- dioxoisoindolin-5- yl)glycyl)piperidin-4-yl)-N-methyl- 1,4,6,7-tetrahydro-5H-pyrazolo[4,3- c]pyridine-5-carboxamide |
| P-002 | 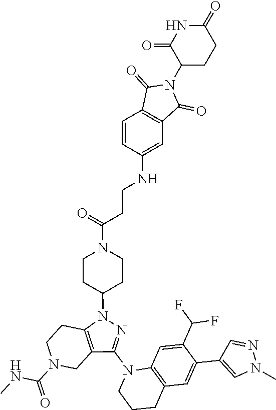 | 3-(7-(difluoromethyl)-6-(1-methyl- 1H-pyrazol-4-yl)-3,4- dihydroquinolin-1(2H)-yl)-1-(1-(3- ((2-(2,6-dioxopiperidin-3-yl)-1,3- dioxoisoindolin-5- yl)amino)propanoyl)piperidin-4-yl)- N-methyl-1,4,6,7-tetrahydro-5H- pyrazolo[4,3-c]pyridine-5- carboxamide |
| P-003 | 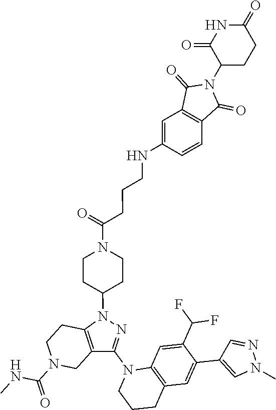 | 3-(7-(difluoromethyl)-6-(1-methyl- 1H-pyrazol-4-yl)-3,4- dihydroquinolin-1(2H)-yl)-1-(1-(4- ((2-(2,6-dioxopiperidin-3-yl)-1,3- dioxoisoindolin-5- yl)amino)butanoyl)piperidin-4-yl)- N-methyl-1,4,6,7-tetrahydro-5H- pyrazolo[4,3-c]pyridine-5- carboxamide |
| P-004 | 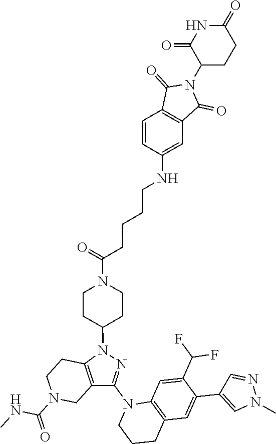 | 3-(7-(difluoromethyl)-6-(1-methyl- 1H-pyrazol-4-yl)-3,4- dihydroquinolin-1(2H)-yl)-1-(1-(5- ((2-(2,6-dioxopiperidin-3-yl)-1,3- dioxoisoindolin-5- yl)amino)pentanoyl)piperidin-4-yl)- N-methyl-1,4,6,7-tetrahydro-5H- pyrazolo[4,3-c]pyridine-5- carboxamide |
| P-005 | 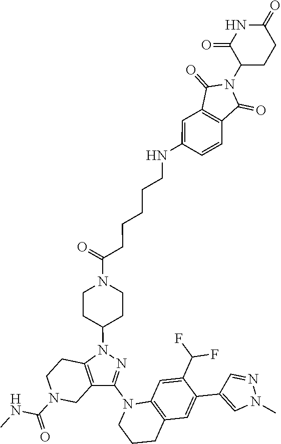 [SMILES: CNC(=O)N1CCc2c(c(N3CCCc4cc(-c5cnn(C)c5)c(C(F)F)cc43)nn2C2CCN(C(=O)CCCCCNc3ccc4c(c3)C(=O)N(C3CCC(=O)NC3=O)C4=O)CC2)C1] | 3-(7-(difluoromethyl)-6-(1-methyl- 1H-pyrazol-4-yl)-3,4- dihydroquinolin-1(2H)-yl)-1-(1-(6- ((2-(2,6-dioxopiperidin-3-yl)-1,3- dioxoisoindolin-5- yl)amino)hexanoyl)piperidin-4-yl)- N-methyl-1,4,6,7-tetrahydro-5H- pyrazolo[4,3-c]pyridine-5- carboxamide |
| P-006 | 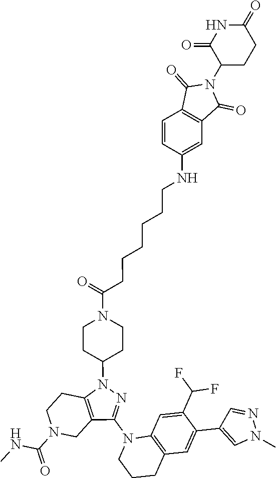 | 3-(7-(difluoromethyl)-6-(1-methyl- 1H-pyrazol-4-yl)-3,4- dihydroquinolin-1(2H)-yl)-1-(1-(7- ((2-(2,6-dioxopiperidin-3-yl)-1,3- dioxoisoindolin-5- yl)amino)heptanoyl)piperidin-4-yl)- N-methyl-1,4,6,7-tetrahydro-5H- pyrazolo[4,3-c]pyridine-5- carboxamide |
| P-007 | 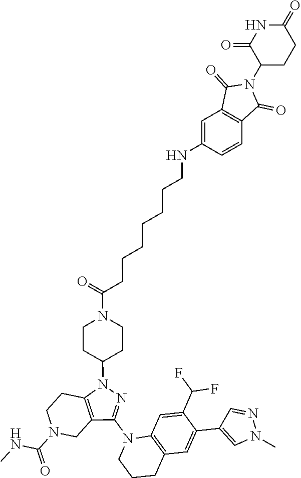 [SMILES: CNC(=O)N1CCc2c(c(N3CCCc4cc(-c5cnn(C)c5)c(C(F)F)cc43)nn2C2CCN(C(=O)CCCCCCCNc3ccc4c(c3)C(=O)N(C3CCC(=O)NC3=O)C4=O)CC2)C1] | 3-(7-(difluoromethyl)-6-(1-methyl- 1H-pyrazol-4-yl)-3,4- dihydroquinolin-1(2H)-yl)-1-(1-(8- ((2-(2,6-dioxopiperidin-3-yl)-1,3- dioxoisoindolin-5- yl)amino)octanoyl)piperidin-4-yl)- N-methyl-1,4,6,7-tetrahydro-5H- pyrazolo[4,3-c]pyridine-5- carboxamide |
| P-008 | 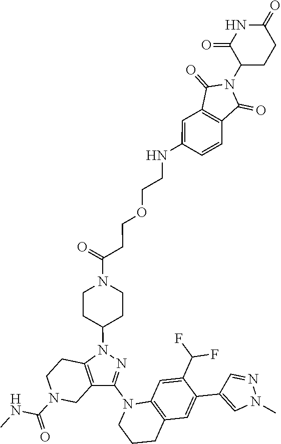 [SMILES: CNC(=O)N1CCc2c(c(N3CCCc4cc(-c5cnn(C)c5)c(C(F)F)cc43)nn2C2CCN(C(=O)CCOCCNc3ccc4c(c3)C(=O)N(C3CCC(=O)NC3=O)C4=O)CC2)C1] | 3-(7-(difluoromethyl)-6-(1-methyl- 1H-pyrazol-4-yl)-3,4- dihydroquinolin-1(2H)-yl)-1-(1-(3- (2-((2-(2,6-dioxopiperidin-3-yl)-1,3- dioxoisoindolin-5-yl) amino)ethoxy)propanoyl)piperidin- 4-yl)-N-methyl-1,4,6,7-tetrahydro- 5H-pyrazolo[4,3-c]pyridine-5- carboxamide |
| P-009 | 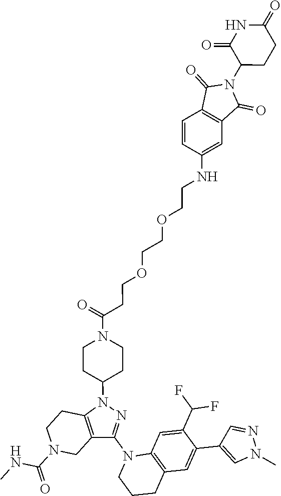 [SMILES: CNC(=O)N1CCc2c(c(N3CCCc4cc(-c5cnn(C)c5)c(C(F)F)cc43)nn2C2CCN(C(=O)CCOCCOCCNc3ccc4c(c3)C(=O)N(C3CCC(=O)NC3=O)C4=O)CC2)C1] | 3-(7-(difluoromethyl)-6-(1-methyl- 1H-pyrazol-4-yl)-3,4- dihydroquinolin-1(2H)-yl)-1-(1-(3- (2-(2-((2-(2,6-dioxopiperidin-3-yl)- 1,3-dioxoisoindolin-5- yl)amino)ethoxy)ethoxy)propanoyl) piperidin-4-yl)-N-methyl-1,4,6,7- tetrahydro-5H-pyrazolo[4,3- c]pyridine-5-carboxamide |
| P-010 | 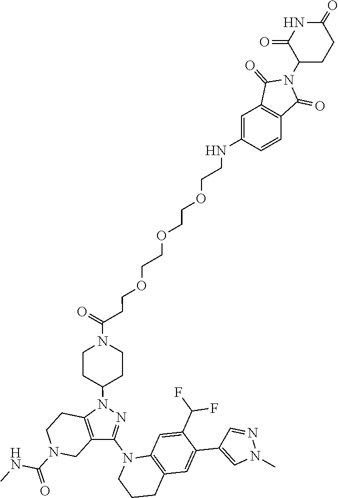 [SMILES: CNC(=O)N1CCc2c(c(N3CCCc4cc(-c5cnn(C)c5)c(C(F)F)cc43)nn2C2CCN(C(=O)CCOCCOCCOCCNc3ccc4c(c3)C(=O)N(C3CCC(=O)NC3=O)C4=O)CC2)C1] | 3-(7-(difluoromethyl)-6-(1-methyl- 1H-pyrazol-4-yl)-3,4- dihydroquinolin-1(2H)-yl)-1-(1-(3- (2-(2-(2-((2-(2,6-dioxopiperidin-3- yl)-1,3-dioxoisoindolin-5- yl)amino)ethoxy)ethoxy)ethoxy) propanoyl)piperidin-4-yl)-N-methyl- 1,4,6,7-tetrahydro-5H-pyrazolo[4,3- c]pyridine-5-carboxamide |
| P-011 | 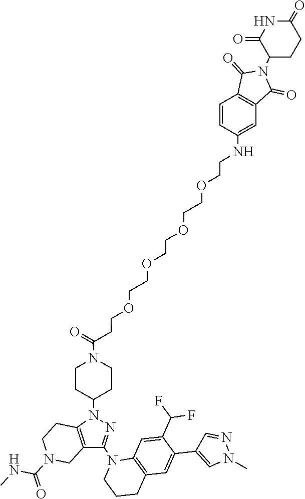 [SMILES: CNC(=O)N1CCc2c(c(N3CCCc4cc(-c5cnn(C)c5)c(C(F)F)cc43)nn2C2CCN(C(=O)CCOCCOCCOCCOCCNc3ccc4c(c3)C(=O)N(C3CCC(=O)NC3=O)C4=O)CC2)C1] | 3-(7-(difluoromethyl)-6-(1-methyl- 1H-pyrazol-4-yl)-3,4- dihydroquinolin-1(2H)-yl)-1-(1-(1- ((2-(2,6-dioxopiperidin-3-yl)-1,3- dioxoisoindolin-5-yl)amino)- 3,6,9,12-tetraoxapentadecan-15- oyl)piperidin-4-yl)-N-methyl- 1,4,6,7-tetrahydro-5H-pyrazolo[4,3- c]pyridine-5-carboxamide |
| P-012 | 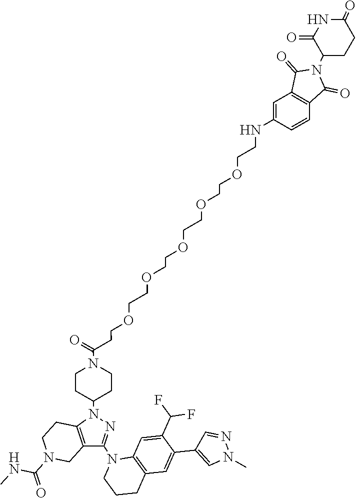 [SMILES: CNC(=O)N1CCc2c(c(N3CCCc4cc(-c5cnn(C)c5)c(C(F)F)cc43)nn2C2CCN(C(=O)CCOCCOCCOCCOCCOCCNc3ccc4c(c3)C(=O)N(C3CCC(=O)NC3=O)C4=O)CC2)C1] | 3-(7-(difluoromethyl)-6-(1-methyl- 1H-pyrazol-4-yl)-3,4- dihydroquinolin-1(2H)-yl)-1-(1-(1- ((2-(2,6-dioxopiperidin-3-yl)-1,3- dioxoisoindolin-5-yl)amino)- 3,6,9,12,15-pentaoxaoctadecan-18- oyl)piperidin-4-yl)-N-methyl- 1,4,6,7-tetrahydro-5H-pyrazolo[4,3- c]pyridine-5-carboxamide |
| P-013 | 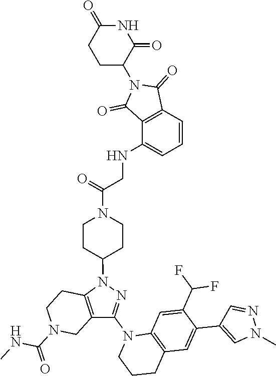 | 3-(7-(difluoromethyl)-6-(1-methyl- 1H-pyrazol-4-yl)-3,4- dihydroquinolin-1(2H)-yl)-1-(1-((2- (2,6-dioxopiperidin-3-yl)-1,3- dioxoisoindolin-4- yl)glycyl)piperidin-4-yl)-N-methyl- 1,4,6,7-tetrahydro-5H-pyrazolo[4,3- c]pyridine-5-carboxamide |
| P-014 | 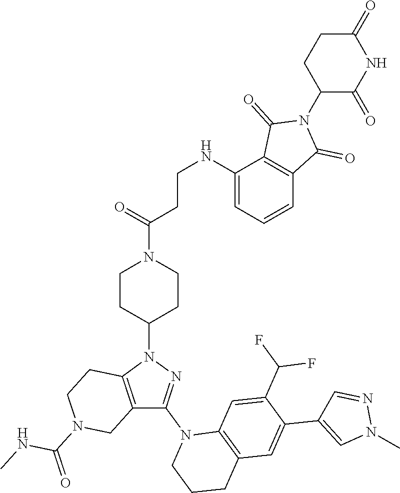 | 3-(7-(difluoromethyl)-6-(1-methyl- 1H-pyrazol-4-yl)-3,4- dihydroquinolin-1(2H)-yl)-1-(1-(3- ((2-(2,6-dioxopiperidin-3-yl)-1,3- dioxoisoindolin-4- yl)amino)propanoyl)piperidin-4-yl)- N-methyl-1,4,6,7-tetrahydro-5H- pyrazolo[4,3-c]pyridine-5- carboxamide |
| P-015 | 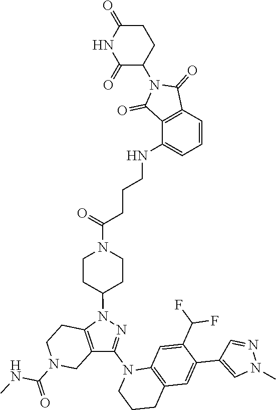 | 3-(7-(difluoromethyl)-6-(1-methyl- 1H-pyrazol-4-yl)-3,4- dihydroquinolin-1(2H)-yl)-1-(1-(4- ((2-(2,6-dioxopiperidin-3-yl)-1,3- dioxoisoindolin-4- yl)amino)butanoyl)piperidin-4-yl)- N-methyl-1,4,6,7-tetrahydro-5H- pyrazolo[4,3-c]pyridine-5- carboxamide |
| P-016 | 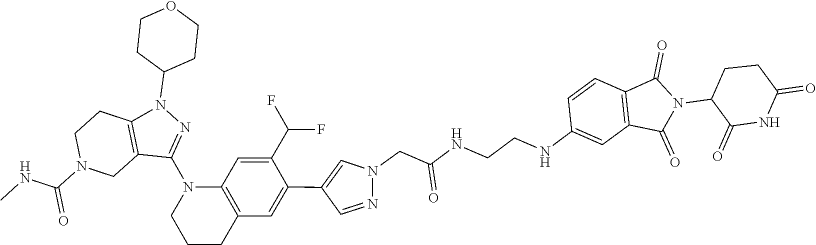 [SMILES: CNC(=O)N1CCc2c(c(N3CCCc4cc(-c5cnn(CC(=O)NCCNc6ccc7c(c6)C(=O)N(C6CCC(=O)NC6=O)C7=O)c5)c(C(F)F)cc43)nn2C2CCOCC2)C1] | 3-(7-(difluoromethyl)-6-(1-(2-((2- ((2-(2,6-dioxopiperidin-3-yl)-1,3- dioxoisoindolin-5- yl)amino)ethyl)amino)-2-oxoethyl)- 1H-pyrazol-4-yl)-3,4- dihydroquinolin-1(2H)-yl)-N- methyl-1-(tetrahydro-2H-pyran-4- yl)-1,4,6,7-tetrahydro-5H- pyrazolo[4,3-c]pyridine-5- carboxamide |
| P-017 | 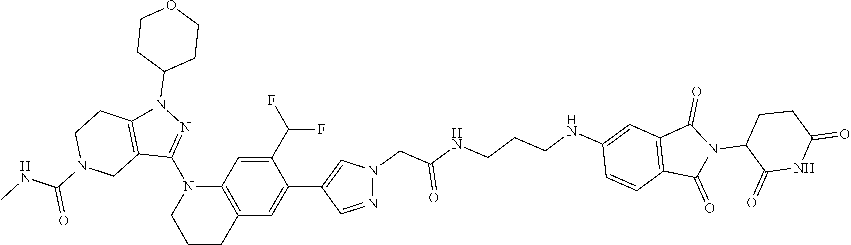 [SMILES: CNC(=O)N1CCc2c(c(N3CCCc4cc(-c5cnn(CC(=O)NCCCNc6ccc7c(c6)C(=O)N(C6CCC(=O)NC6=O)C7=O)c5)c(C(F)F)cc43)nn2C2CCOCC2)C1] | 3-(7-(difluoromethyl)-6-(1-(2-((3- ((2-(2,6-dioxopiperidin-3-yl)-1,3- dioxoisoindolin-5- yl)amino)propyl)amino)-2- oxoethyl)-1H-pyrazol-4-yl)-3,4- dihydroquinolin-1(2H)-yl)-N- methyl-1-(tetrahydro-2H-pyran-4- yl)-1,4,6,7-tetrahydro-5H- pyrazolo[4,3-c]pyridine-5- carboxamide |
| P-018 | 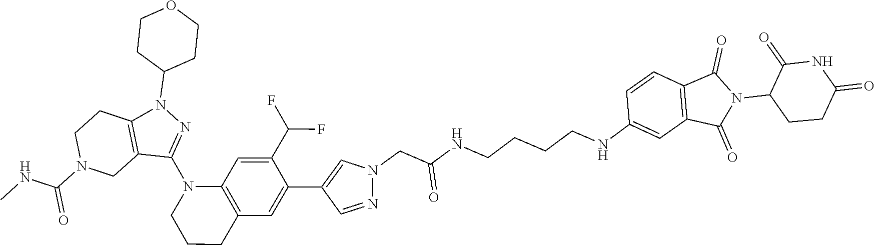 [SMILES: CNC(=O)N1CCc2c(c(N3CCCc4cc(-c5cnn(CC(=O)NCCCCNc6ccc7c(c6)C(=O)N(C6CCC(=O)NC6=O)C7=O)c5)c(C(F)F)cc43)nn2C2CCOCC2)C1] | 3-(7-(difluoromethyl)-6-(1-(2-((4- ((2-(2,6-dioxopiperidin-3-yl)-1,3- dioxoisoindolin-5- yl)amino)butyl)amino)-2-oxoethyl)- 1H-pyrazol-4-yl)-3,4- dihydroquinolin-1(2H)-yl)-N- methyl-1-(tetrahydro-2H-pyran-4- yl)-1,4,6,7-tetrahydro-5H- pyrazolo[4,3-c]pyridine-5- carboxamide |
| P-019 | 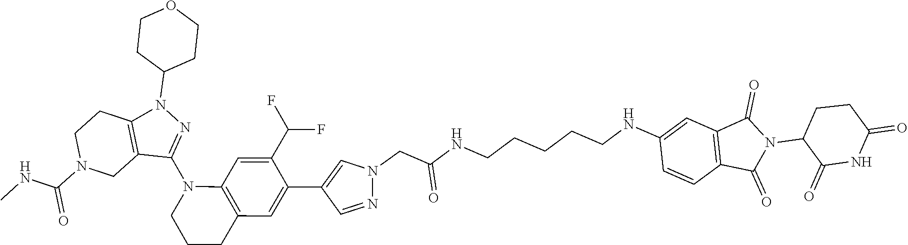 [SMILES: CNC(=O)N1CCc2c(c(N3CCCc4cc(-c5cnn(CC(=O)NCCCCCNc6ccc7c(c6)C(=O)N(C6CCC(=O)NC6=O)C7=O)c5)c(C(F)F)cc43)nn2C2CCOCC2)C1] | 3-(7-(difluoromethyl)-6-(1-(2-((5- ((2-(2,6-dioxopiperidin-3-yl)-1,3- dioxoisoindolin-5- yl)amino)pentyl)amino)-2- oxoethyl)-1H-pyrazol-4-yl)-3,4- dihydroquinolin-1(2H)-yl)-N- methyl-1-(tetrahydro-2H-pyran-4- yl)-1,4,6,7-tetrahydro-5H- pyrazolo[4,3-c]pyridine-5- carboxamide |
| P-020 | 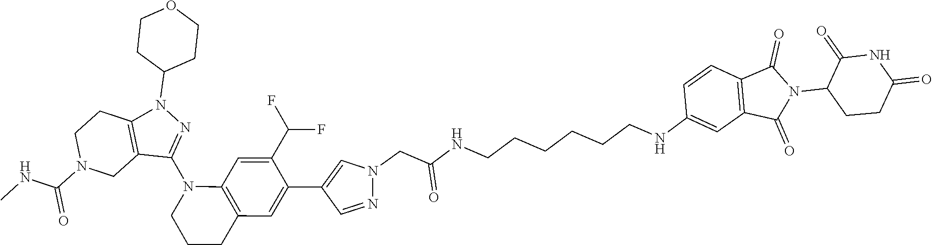 [SMILES: CNC(=O)N1CCc2c(c(N3CCCc4cc(-c5cnn(CC(=O)NCCCCCCNc6ccc7c(c6)C(=O)N(C6CCC(=O)NC6=O)C7=O)c5)c(C(F)F)cc43)nn2C2CCOCC2)C1] | 3-(7-(difluoromethyl)-6-(1-(2-((6- ((2-(2,6-dioxopiperidin-3-yl)-1,3- dioxoisoindolin-5- yl)amino)hexyl)amino)-2-oxoethyl)- 1H-pyrazol-4-yl)-3,4- dihydroquinolin-1(2H)-yl)-N- methyl-1-(tetrahydro-2H-pyran-4- yl)-1,4,6,7-tetrahydro-5H- pyrazolo[4,3-c]pyridine-5- carboxamide |
| P-021 | 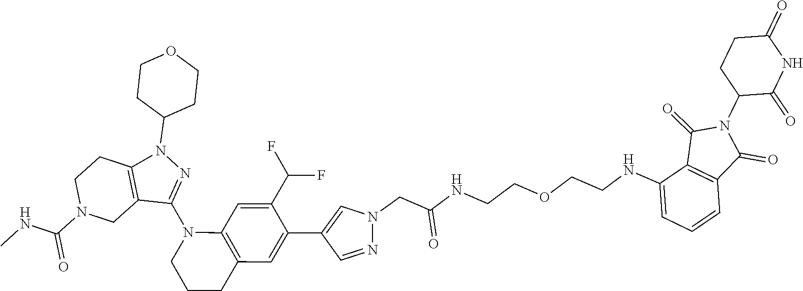 [SMILES: CNC(=O)N1CCc2c(c(N3CCCc4cc(-c5cnn(CC(=O)NCCOCCNc6cccc7c6C(=O)N(C6CCC(=O)NC6=O)C7=O)c5)c(C(F)F)cc43)nn2C2CCOCC2)C1] | 3-(7-(difluoromethyl)-6-(1-(2-((2- (2-((2-(2,6-dioxopiperidin-3-yl)-1,3- dioxoisoindolin-4- yl)amino)ethoxy)ethyl)amino)-2- oxoethyl)-1H-pyrazol-4-yl)-3,4- dihydroquinolin-1(2H)-yl))-N- methyl-1-(tetrahydro-2H-pyran-4- yl)-1,4,6,7-tetrahydro-5H- pyrazolo[4,3-c]pyridine-5- carboxamide |
| P-022 | 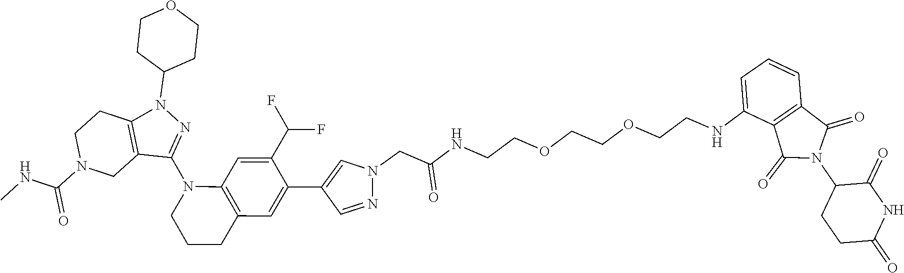 [SMILES: CNC(=O)N1CCc2c(c(N3CCCc4cc(-c5cnn(CC(=O)NCCOCCOCCNc6cccc7c6C(=O)N(C6CCC(=O)NC6=O)C7=O)c5)c(C(F)F)cc43)nn2C2CCOCC2)C1] | 3-(7-(difluoromethyl)-6-(1-(2-((2- (2-(2-((2-(2,6-dioxopiperidin-3-yl)- 1,3-dioxoisoindolin-4-yl) amino)ethoxy)ethoxy)ethyl)amino)- 2-oxoethyl)-1H-pyrazol-4-yl)- 3,4-dihydroquinolin-1(2H)-yl)-N- methyl-1-(tetrahydro-2H-pyran-4- yl)-1,4,6,7-tetrahydro-5H- pyrazolo[4,3-c]pyridine-5- carboxamide |
| P-023 | 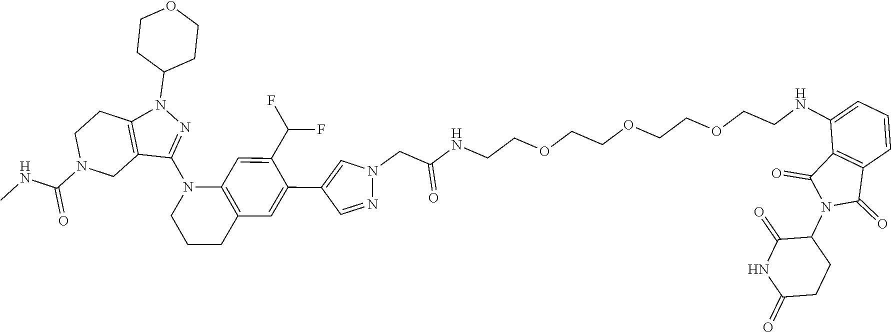 [SMILES: CNC(=O)N1CCc2c(c(N3CCCc4cc(-c5cnn(CC(=O)NCCOCCOCCOCCNc6cccc7c6C(=O)N(C6CCC(=O)NC6=O)C7=O)c5)c(C(F)F)cc43)nn2C2CCOCC2)C1] | 3-(7-(difluoromethyl)-6-(1-(14-((2- (2,6-dioxopiperidin-3-yl)-1,3- dioxoisoindolin-4-yl)amino)-2-oxo- 6,9,12-trioxa-3-azatetradecyl)-1H- pyrazol-4-yl)-3,4-dihydroquinolin- 1(2H)-yl)-N-methyl-1-(tetrahydro- 2H-pyran-4-yl)-1,4,6,7-tetrahydro- 5H-pyrazolo[4,3-c]pyridine-5- carboxamide |
| P-024 | 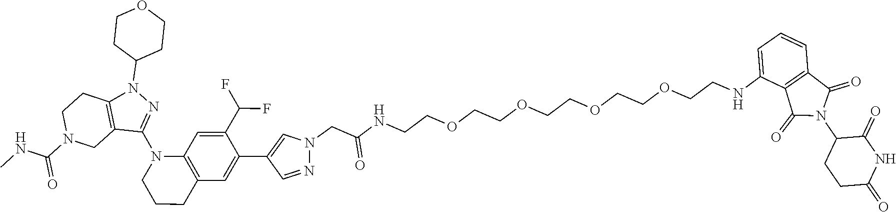 [SMILES: CNC(=O)N1CCc2c(c(N3CCCc4cc(-c5cnn(CC(=O)NCCOCCOCCOCCOCCNc6cccc7c6C(=O)N(C6CCC(=O)NC6=O)C7=O)c5)c(C(F)F)cc43)nn2C2CCOCC2)C1] | 3-(7-(difluoromethyl)-6-(1-(17-((2- (2,6-dioxopiperidin-3-yl)-1,3- dioxoisoindolin-4-yl)amino)-2-oxo- 6,9,12,15-tetraoxa-3- azaheptadecyl)-1H-pyrazol-4-yl)- 3,4-dihydroquinolin-1(2H)-yl)-N- methyl-1-(tetrahydro-2H-pyran-4- yl)-1,4,6,7-tetrahydro-5H- pyrazolo[4,3-c]pyridine-5- carboxamide |
| P-025 | 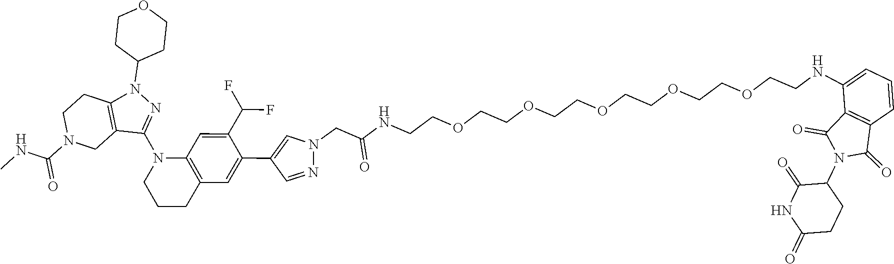 [SMILES: CNC(=O)N1CCc2c(c(N3CCCc4cc(-c5cnn(CC(=O)NCCOCCOCCOCCOCCOCCNc6cccc7c6C(=O)N(C6CCC(=O)NC6=O)C7=O)c5)c(C(F)F)cc43)nn2C2CCOCC2)C1] | 3-(7-(difluoromethyl)-6-(1-(20-((2- (2,6-dioxopiperidin-3-yl)-1,3- dioxoisoindolin-4-yl)amino)-2-oxo- 6,9,12,15,18-pentaoxa-3-azaicosyl)- 1H-pyrazol-4-yl)-3,4- dihydroquinolin-1(2H)-yl)-N- methyl-1-(tetrahydro-2H-pyran-4- yl)-1,4,6,7-tetrahydro-5H- pyrazolo[4,3-c]pyridine-5- carboxamide |
| P-026 | 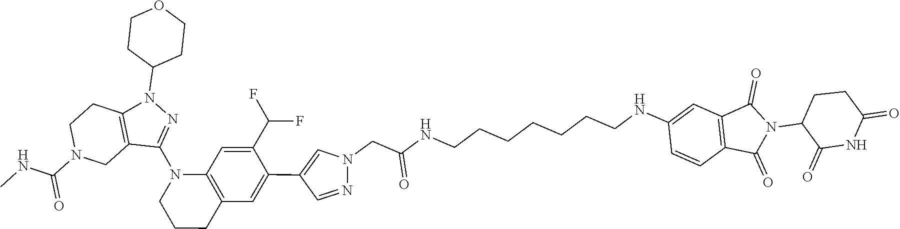 [SMILES: CNC(=O)N1CCc2c(c(N3CCCc4cc(-c5cnn(CC(=O)NCCCCCCCNc6ccc7c(c6)C(=O)N(C6CCC(=O)NC6=O)C7=O)c5)c(C(F)F)cc43)nn2C2CCOCC2)C1] | 3-(7-(difluoromethyl)-6-(1-(2-((7- ((2-(2,6-dioxopiperidin-3-yl)-1,3- dioxoisoindolin-5- yl)amino)heptyl)amino)-2- oxoethyl)-1H-pyrazol-4-yl)-3,4- dihydroquinolin-1(2H)-yl)-N- methyl-1-(tetrahydro-2H-pyran-4- yl)-1,4,6,7-tetrahydro-5H- pyrazolo[4,3-c]pyridine-5- carboxamide |
| P-027 | 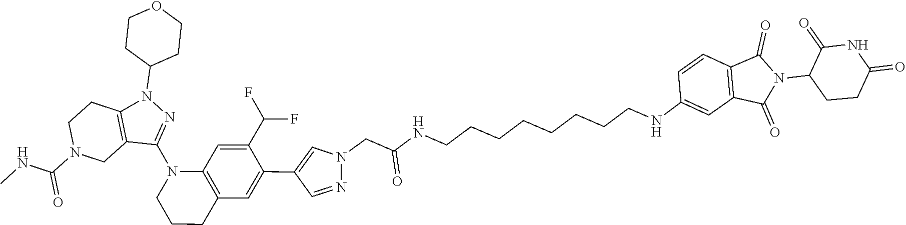 [SMILES: CNC(=O)N1CCc2c(c(N3CCCc4cc(-c5cnn(CC(=O)NCCCCCCCCNc6ccc7c(c6)C(=O)N(C6CCC(=O)NC6=O)C7=O)c5)c(C(F)F)cc43)nn2C2CCOCC2)C1] | 3-(7-(difluoromethyl)-6-(1-(2-((8- ((2-(2,6-dioxopiperidin-3-yl)-1,3- dioxoisoindolin-5- yl)amino)octyl)amino)-2-oxoethyl)- dihydroquinolin-1(2H)-yl)-N- methyl-1-(tetrahydro-2H-pyran-4- yl)-1,4,6,7-tetrahydro-5H- pyrazolo[4,3-c]pyridine-5- carboxamide |
| P-028 | 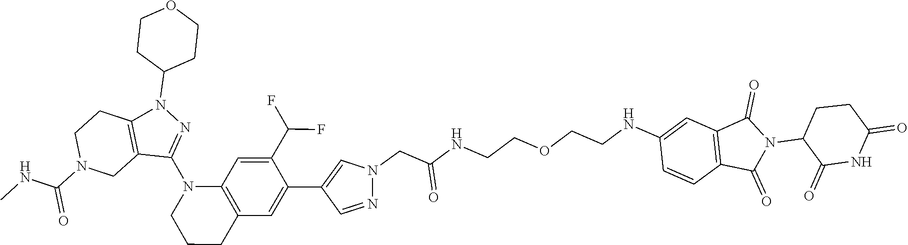 [SMILES: CNC(=O)N1CCc2c(c(N3CCCc4cc(-c5cnn(CC(=O)NCCOCCNc6ccc7c(c6)C(=O)N(C6CCC(=O)NC6=O)C7=O)c5)c(C(F)F)cc43)nn2C2CCOCC2)C1] | 3-(7-(difluoromethyl)-6-(1-(2-((2- (2-((2-(2,6-dioxopiperidin-3-yl)-1,3- dioxoisoindolin-5- yl)amino)ethoxy)ethyl)amino)-2- oxoethyl)-1H-pyrazol-4-yl)-3,4- dihydroquinolin-1(2H)-yl)-N- methyl-1-(tetrahydro-2H-pyran-4- yl)-1,4,6,7-tetrahydro-5H- pyrazolo[4,3-c]pyridine-5- carboxamide |
| P-029 | 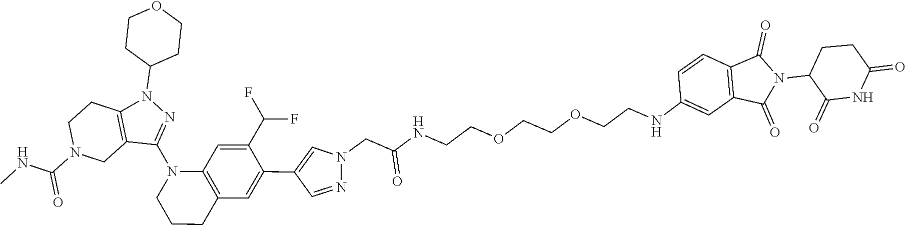 [SMILES: CNC(=O)N1CCc2c(c(N3CCCc4cc(-c5cnn(CC(=O)NCCOCCOCCNc6ccc7c(c6)C(=O)N(C6CCC(=O)NC6=O)C7=O)c5)c(C(F)F)cc43)nn2C2CCOCC2)C1] | 3-(7-(difluoromethyl)-6-(1-(2-((2- (2-(2-((2-(2,6-dioxopiperidin-3-yl)- 1,3-dioxoisoindolin-5-yl) amino)ethoxy)ethoxy)ethyl)amino)- 2-oxoethyl)-1H-pyrazol-4-yl)- 3,4-dihydroquinolin-1(2H)-yl)-N- methyl-1-(tetrahydro-2H-pyran-4- yl)-1,4,6,7-tetrahydro-5H- pyrazolo[4,3-c]pyridine-5- carboxamide |
| P-030 | 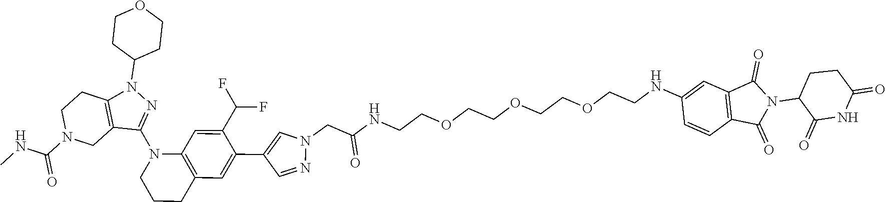 [SMILES: CNC(=O)N1CCc2c(c(N3CCCc4cc(-c5cnn(CC(=O)NCCOCCOCCOCCNc6ccc7c(c6)C(=O)N(C6CCC(=O)NC6=O)C7=O)c5)c(C(F)F)cc43)nn2C2CCOCC2)C1] | 3-(7-(difluoromethyl)-6-(1-(14-((2- (2,6-dioxopiperidin-3-yl)-1,3- dioxoisoindolin-5-yl)amino)-2-oxo- 6,9,12-trioxa-3-azatetradecyl)-1H- pyrazol-4-yl)-3,4-dihydroquinolin- 1(2H)-yl)-N-methyl-1-(tetrahydro- 2H-pyran-4-yl)-1,4,6,7-tetrahydro- 5H-pyrazolo[4,3-c]pyridine-5- carboxamide |
| P-031 | 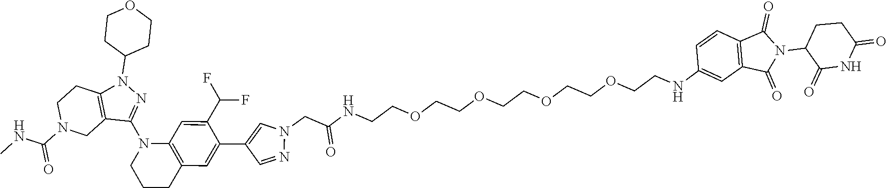 [SMILES: CNC(=O)N1CCc2c(c(N3CCCc4cc(-c5cnn(CC(=O)NCCOCCOCCOCCOCCNc6ccc7c(c6)C(=O)N(C6CCC(=O)NC6=O)C7=O)c5)c(C(F)F)cc43)nn2C2CCOCC2)C1] | 3-(7-(difluoromethyl)-6-(1-(17-((2- (2,6-dioxopiperidin-3-yl)-1,3- dioxoisoindolin-5-yl)amino)-2-oxo- 6,9,12,15-tetraoxa-3- azaheptadecyl)-1H-pyrazol-4-yl)- 3,4-dihydroquinolin-1(2H)-yl)-N- methyl-1-(tetrahydro-2H-pyran-4- yl)-1,4,6,7-tetrahydro-5H- pyrazolo[4,3-c]pyridine-5- carboxamide |
| P-032 | 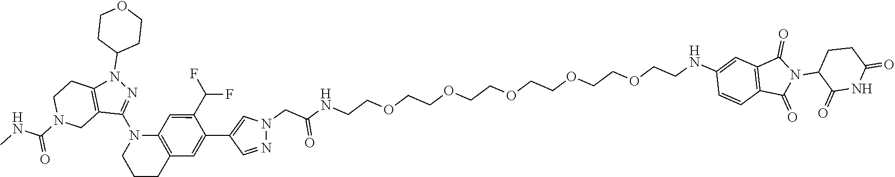 [SMILES: CNC(=O)N1CCc2c(c(N3CCCc4cc(-c5cnn(CC(=O)NCCOCCOCCOCCOCCOCCNc6ccc7c(c6)C(=O)N(C6CCC(=O)NC6=O)C7=O)c5)c(C(F)F)cc43)nn2C2CCOCC2)C1] | 3-(7-(difluoromethyl)-6-(1-(20-((2- (2,6-dioxopiperidin-3-yl)-1,3- dioxoisoindolin-5-yl)amino)-2-oxo- 6,9,12,15,18-pentaoxa-3-azaicosyl)- 1H-pyrazol-4-yl)-3,4- dihydroquinolin-1(2H)-yl)-N- methyl-1-(tetrahydro-2H-pyran-4- yl)-1,4,6,7-tetrahydro-5H- pyrazolo[4,3-c]pyridine-5- carboxamide |
| P-033 | 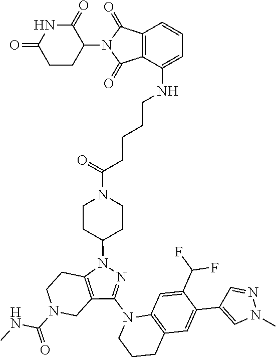 | 3-(7-(difluoromethyl)-6-(1-methyl- 1H-pyrazol-4-yl)-3,4- dihydroquinolin-1(2H)-yl)-1-(1-(5- ((2-(2,6-dioxopiperidin-3-yl)-1,3- dioxoisoindolin-4- yl)amino)pentanoyl)piperidin-4-yl)- N-methyl-1,4,6,7-tetrahydro-5H- pyrazolo[4,3-c]pyridine-5- carboxamide |
| P-034 | 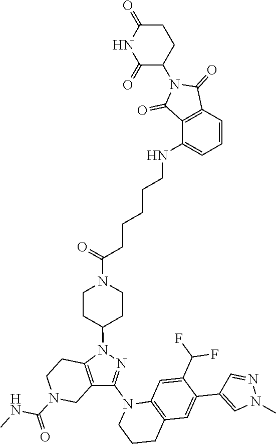 | 3-(7-(difluoromethyl)-6-(1-methyl- 1H-pyrazol-4-yl)-3,4- dihydroquinolin-1(2H)-yl)-1-(1-(6- ((2-(2,6-dioxopiperidin-3-yl)-1,3- dioxoisoindolin-4- yl)amino)hexanoyl)piperidin-4-yl)- N-methyl-1,4,6,7-tetrahydro-5H- pyrazolo[4,3-c]pyridine-5- carboxamide |
| P-035 | 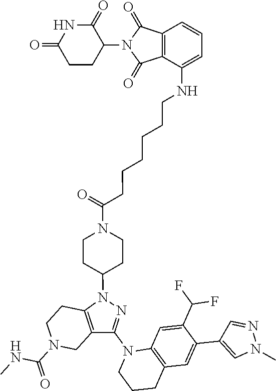 | 3-(7-(difluoromethyl)-6-(1-methyl- 1H-pyrazol-4-yl)-3,4- dihydroquinolin-1(2H)-yl)-1-(1-(7- ((2-(2,6-dioxopiperidin-3-yl)-1,3- dioxoisoindolin-4- yl)amino)heptanoyl)piperidin-4-yl)- N-methyl-1,4,6,7-tetrahydro-5H- pyrazolo[4,3-c]pyridine-5- carboxamide |
| P-036 | 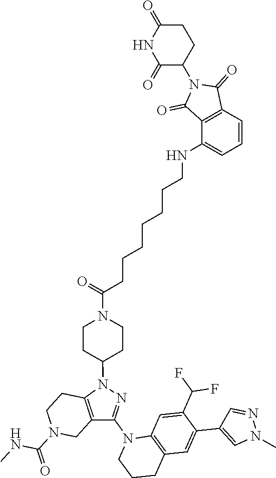 | 3-(7-(difluoromethyl)-6-(1-methyl- 1H-pyrazol-4-yl)-3,4- dihydroquinolin-1(2H)-yl)-1-(1-(8- ((2-(2,6-dioxopiperidin-3-yl)-1,3- dioxoisoindolin-4- yl)amino)octanoyl)piperidin-4-yl)- N-methyl-1,4,6,7-tetrahydro-5H- pyrazolo[4,3-c]pyridine-5- carboxamide |
| P-037 | 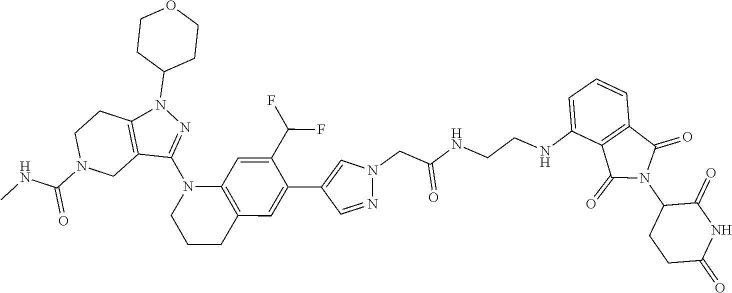 [SMILES: CNC(=O)N1CCc2c(c(N3CCCc4cc(-c5cnn(CC(=O)NCCNc6cccc7c6C(=O)N(C6CCC(=O)NC6=O)C7=O)c5)c(C(F)F)cc43)nn2C2CCOCC2)C1] | 3-(7-(difluoromethyl)-6-(1-(2-((2- ((2-(2,6-dioxopiperidin-3-yl)-1,3- dioxoisoindolin-4- yl)amino)ethyl)amino)-2-oxoethyl)- 1H-pyrazol-4-yl)-3,4- dihydroquinolin-1(2H)-yl)-N- methyl-1-(tetrahydro-2H-pyran-4- yl)-1,4,6,7-tetrahydro-5H- pyrazolo[4,3-c]pyridine-5- carboxamide |
| P-038 | 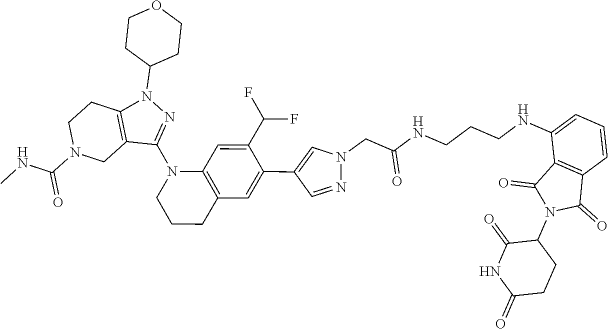 [SMILES: CNC(=O)N1CCc2c(c(N3CCCc4cc(-c5cnn(CC(=O)NCCCNc6cccc7c6C(=O)N(C6CCC(=O)NC6=O)C7=O)c5)c(C(F)F)cc43)nn2C2CCOCC2)C1] | 3-(7-(difluoromethyl)-6-(1-(2-((3- ((2-(2,6-dioxopiperidin-3-yl)-1,3- dioxoisoindolin-4- yl)amino)propyl)amino)-2- oxoethyl)-1H-pyrazol-4-yl)-3,4- dihydroquinolin-1(2H)-yl)-N- methyl-1-(tetrahydro-2H-pyran-4- yl)-1,4,6,7-tetrahydro-5H- pyrazolo[4,3-c]pyridine-5- carboxamide |
| P-039 | 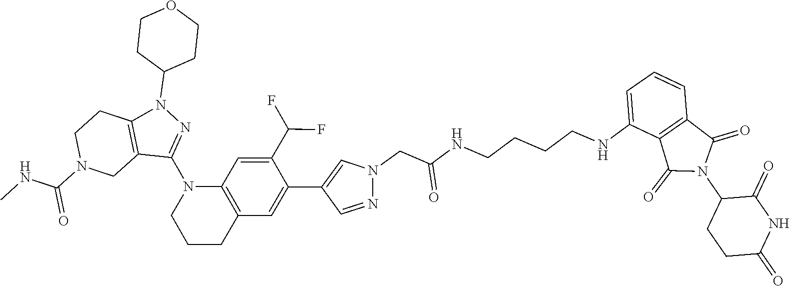 [SMILES: CNC(=O)N1CCc2c(c(N3CCCc4cc(-c5cnn(CC(=O)NCCCCNc6cccc7c6C(=O)N(C6CCC(=O)NC6=O)C7=O)c5)c(C(F)F)cc43)nn2C2CCOCC2)C1] | 3-(7-(difluoromethyl)-6-(1-(2-((4- ((2-(2,6-dioxopiperidin-3-yl)-1,3- dioxoisoindolin-4- yl)amino)butyl)amino)-2-oxoethyl)- 1H-pyrazol-4-yl)-3,4- dihydroquinolin-1(2H)-yl)-N- methyl-1-(tetrahydro-2H-pyran-4- yl)-1,4,6,7-tetrahydro-5H- pyrazolo[4,3-c]pyridine-5- carboxamide |
| P-040 | 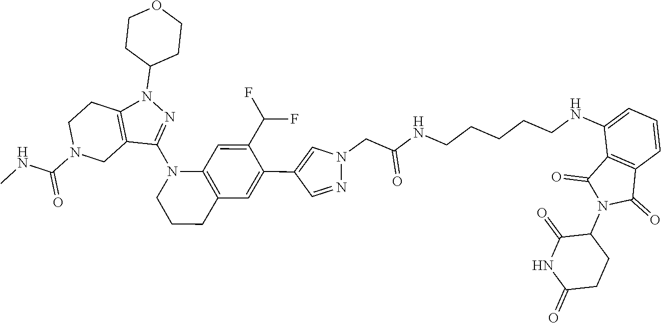 [SMILES: CNC(=O)N1CCc2c(c(N3CCCc4cc(-c5cnn(CC(=O)NCCCCCNc6cccc7c6C(=O)N(C6CCC(=O)NC6=O)C7=O)c5)c(C(F)F)cc43)nn2C2CCOCC2)C1] | 3-(7-(difluoromethyl)-6-(1-(2-((5- ((2-(2,6-dioxopiperidin-3-yl)-1,3- dioxoisoindolin-4- yl)amino)pentyl)amino)-2- oxoethyl)-1H-pyrazol-4-yl)-3,4- dihydroquinolin-1(2H)-yl)-N- methyl-1-(tetrahydro-2H-pyran-4- yl)-1,4,6,7-tetrahydro-5H- pyrazolo[4,3-c]pyridine-5- carboxamide |
| P-041 | 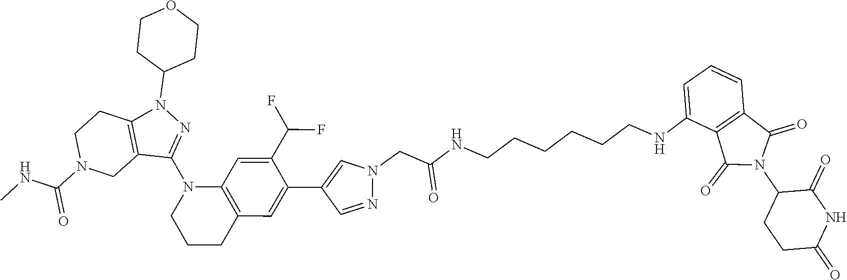 [SMILES: CNC(=O)N1CCc2c(c(N3CCCc4cc(-c5cnn(CC(=O)NCCCCCCNc6cccc7c6C(=O)N(C6CCC(=O)NC6=O)C7=O)c5)c(C(F)F)cc43)nn2C2CCOCC2)C1] | 3-(7-(difluoromethyl)-6-(1-(2-((6- ((2-(2,6-dioxopiperidin-3-yl)-1,3- dioxoisoindolin-4- yl)amino)hexyl)amino)-2-oxoethyl)- 1H-pyrazol-4-yl)-3,4- dihydroquinolin-1(2H)-yl)-N- methyl-1-(tetrahydro-2H-pyran-4- yl)-1,4,6,7-tetrahydro-5H- pyrazolo[4,3-c]pyridine-5- carboxamide |
| P-042 | 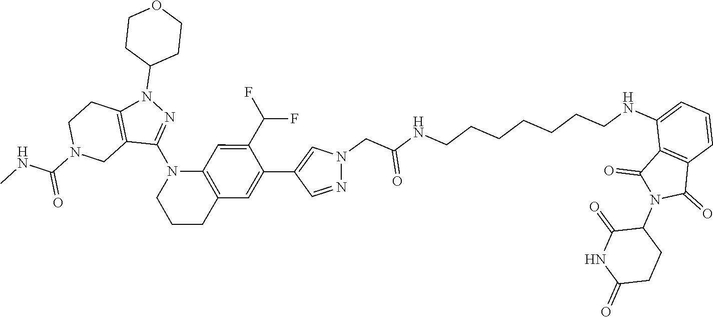 [SMILES: CNC(=O)N1CCc2c(c(N3CCCc4cc(-c5cnn(CC(=O)NCCCCCCCNc6cccc7c6C(=O)N(C6CCC(=O)NC6=O)C7=O)c5)c(C(F)F)cc43)nn2C2CCOCC2)C1] | 3-(7-(difluoromethyl)-6-(1-(2-((7- ((2-(2,6-dioxopiperidin-3-yl)-1,3- dioxoisoindolin-4- yl)amino)heptyl)amino)-2- oxoethyl)-1H-pyrazol-4-yl)-3,4- dihydroquinolin-1(2H)-yl)-N- methyl-1-(tetrahydro-2H-pyran-4- yl)-1,4,6,7-tetrahydro-5H- pyrazolo[4,3-c]pyridine-5- carboxamide |
| P-043 | 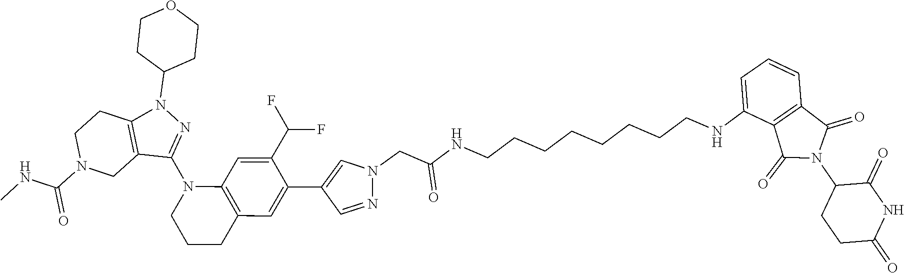 [SMILES: CNC(=O)N1CCc2c(c(N3CCCc4cc(-c5cnn(CC(=O)NCCCCCCCCNc6cccc7c6C(=O)N(C6CCC(=O)NC6=O)C7=O)c5)c(C(F)F)cc43)nn2C2CCOCC2)C1] | 3-(7-(difluoromethyl)-6-(1-(2-((8- ((2-(2,6-dioxopiperidin-3-yl)-1,3- dioxoisoindolin-4- yl)amino)octyl)amino)-2-oxoethyl)- 1H-pyrazol-4-yl)-3,4- dihydroquinolin-1(2H)-yl)-N- methyl-1-(tetrahydro-2H-pyran-4- yl)-1,4,6,7-tetrahydro-5H- pyrazolo[4,3-c]pyridine-5- carboxamide |
| P-044 | 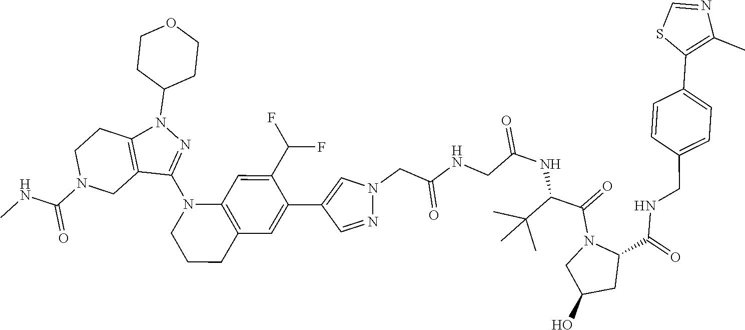 | 3-(7-(difluoromethyl)-6-(1-(2-((2- (((S)-1-((2S,4R)-4-hydroxy-2-((4-(4- methylthiazol-5- yl)benzyl)carbamoyl)pyrrolidin-1- yl)-3,3-dimethyl-1-oxobutan-2- yl)amino)-2-oxoethyl)amino)-2- oxoethyl)-1H-pyrazol-4-yl)-3,4- dihydroquinolin-1(2H)-yl)-N- methyl-1-(tetrahydro-2H-pyran-4- yl)-1,4,6,7-tetrahydro-5H- pyrazolo[4,3-c]pyridine-5- carboxamide |
| P-045 | 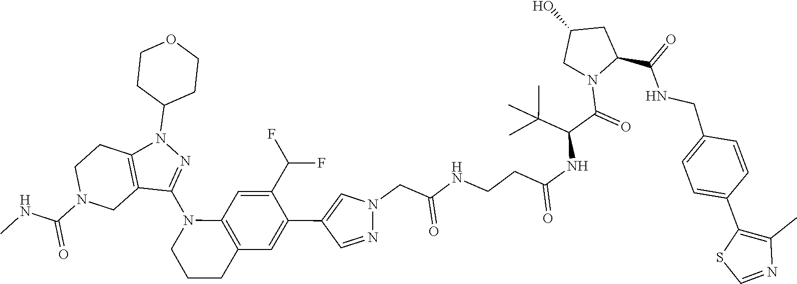 | 3-(7-(difluoromethyl)-6-(1-(2-((3- (((S)-1-((2S,4R)-4-hydroxy-2-((4-(4- methylthiazol-5- yl)benzyl)carbamoyl)pyrrolidin-1- yl)-3,3-dimethyl-1-oxobutan-2- yl)amino)-3-oxopropyl)amino)-2- oxoethyl)-1H-pyrazol-4-yl)-3,4- dihydroquinolin-1(2H)-yl)-N- methyl-1-(tetrahydro-2H-pyran-4- yl)-1,4,6,7-tetrahydro-5H- pyrazolo[4,3-c]pyridine-5- carboxamide |
| P-046 | 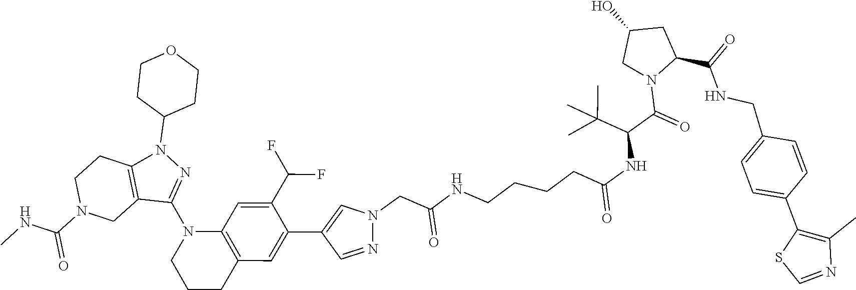 | 3-(7-(difluoromethyl)-6-(1-(2-((5- (((S)-1-((2S,4R)-4-hydroxy-2-((4-(4- methylthiazol-5- yl)benzyl)carbamoyl)pyrrolidin-1- yl)-3,3-dimethyl-1-oxobutan-2- yl)amino)-5-oxopentyl)amino)-2- oxoethyl)-1H-pyrazol-4-yl)-3,4- dihydroquinolin-1(2H)-yl)-N- methyl-1-(tetrahydro-2H-pyran-4- yl)-1,4,6,7-tetrahydro-5H- pyrazolo[4,3-c]pyridine-5- carboxamide |
| P-047 | 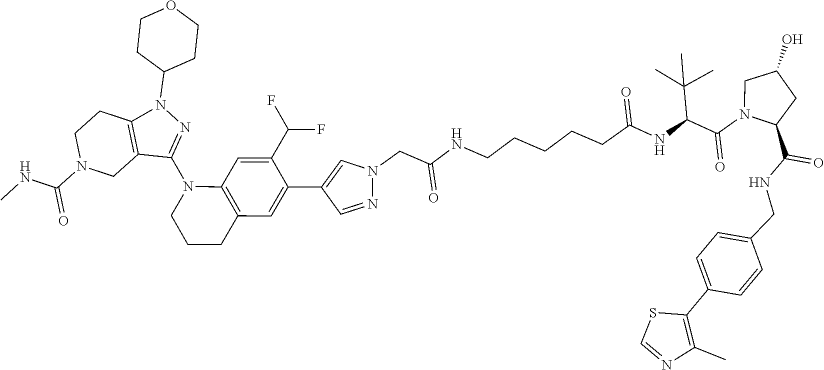 [SMILES: CNC(=O)N1CCc2c(c(N3CCCc4cc(-c5cnn(CC(=O)NCCCCCC(=O)N[C@H](C(=O)N6C[C@H](O)C[C@H]6C(=O)NCc6ccc(-c7scnc7C)cc6)C(C)(C)C)c5)c(C(F)F)cc43)nn2C2CCOCC2)C1] | 3-(7-(difluoromethyl)-6-(1-(2-((6- (((S)-1-((2S,4R)-4-hydroxy-2-((4-(4- methylthiazol-5- yl)benzyl)carbamoyl)pyrrolidin-1- yl)-3,3-dimethyl-1-oxobutan-2- yl)amino)-6-oxohexyl)amino)-2- oxoethyl)-1H-pyrazol-4-yl)-3,4- dihydroquinolin-1(2H)-yl)-N- methyl-1-(tetrahydro-2H-pyran-4- yl)-1,4,6,7-tetrahydro-5H- pyrazolo[4,3-c]pyridine-5- carboxamide |
| P-048 | 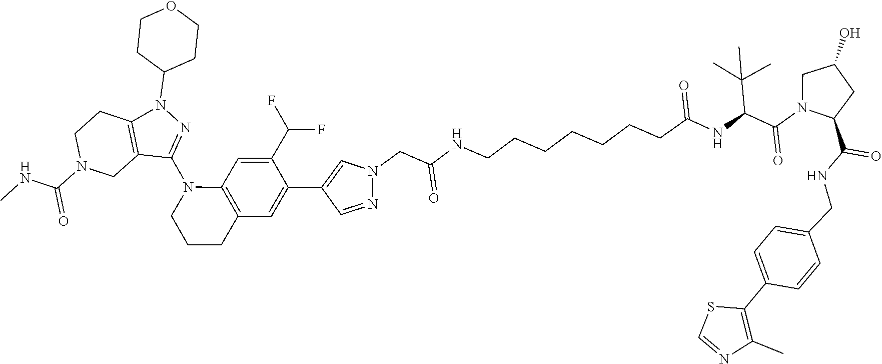 [SMILES: CNC(=O)N1CCc2c(c(N3CCCc4cc(-c5cnn(CC(=O)NCCCCCCCC(=O)N[C@H](C(=O)N6C[C@H](O)C[C@H]6C(=O)NCc6ccc(-c7scnc7C)cc6)C(C)(C)C)c5)c(C(F)F)cc43)nn2C2CCOCC2)C1] | 3-(7-(difluoromethyl)-6-(1-(2-((8- (((S)-1-((2S,4R)-4-hydroxy-2-((4-(4- methylthiazol-5- yl)benzyl)carbamoyl)pyrrolidin-1- yl)-3,3-dimethyl-1-oxobutan-2- yl)amino)-8-oxooctyl)amino)-2- oxoethyl)-1H-pyrazol-4-yl)-3,4- dihydroquinolin-1(2H)-yl)-N- methyl-1-(tetrahydro-2H-pyran-4- yl)-1,4,6,7-tetrahydro-5H- pyrazolo[4,3-c]pyridine-5- carboxamide |
| P-049 | 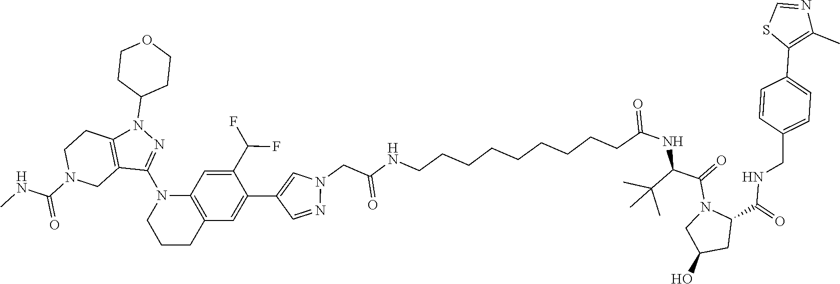 | 3-(7-(difluoromethyl)-6-(1-(2-((10- (((S)-1-((2S,4R)-4-hydroxy-2-((4-(4- methylthiazol-5- yl)benzyl)carbamoyl)pyrrolidin-1- yl)-3,3-dimethyl-1-oxobutan-2- yl)amino)-10-oxodecyl)amino)-2- oxoethyl)-1H-pyrazol-4-yl)-3,4- dihydroquinolin-1(2H)-yl)-N- methyl-1-(tetrahydro-2H-pyran-4- yl)-1,4,6,7-tetrahydro-5H- pyrazolo[4,3-c]pyridine-5- carboxamide |
| P-050 | 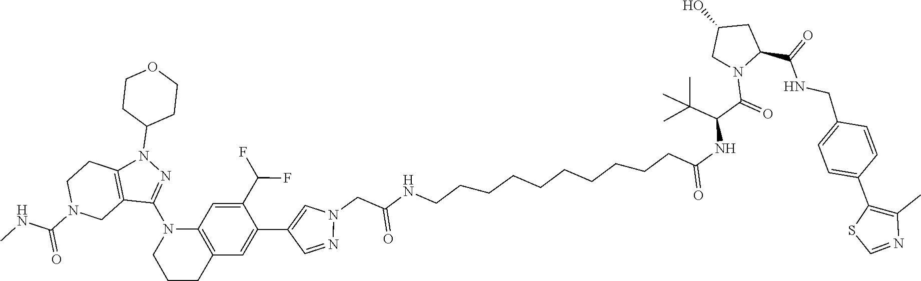 | 3-(7-(difluoromethyl)-6-(1-(2-((11- (((S)-1-((2S,4R)-4-hydroxy-2-((4-(4- methylthiazol-5- yl)benzyl)carbamoyl)pyrrolidin-1- yl)-3,3-dimethyl-1-oxobutan-2- yl)amino)-11-oxoundecyl)amino)-2- oxoethyl)-1H-pyrazol-4-yl)-3,4- dihydroquinolin-1(2H)-yl)-N- methyl-1-(tetrahydro-2H-pyran-4- yl)-1,4,6,7-tetrahydro-5H- pyrazolo[4,3-c]pyridine-5- carboxamide |
| CPD- 051 (P- 064) | 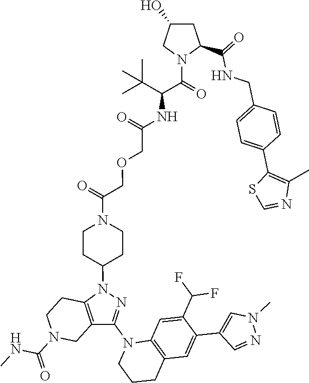 | 3-(7-(difluoromethyl)-6-(1-methyl- 1H-pyrazol-4-yl)-3,4- dihydroquinolin-1(2H)-yl)-1-(1-(2- (2-(((S)-1-((2S,4R)-4-hydroxy-2-((4- (4-methylthiazol-5- yl)benzyl)carbamoyl)pyrrolidin-1- yl)-3,3-dimethyl-1-oxobutan-2- yl)amino)-2- oxoethoxy)acetyl)piperidin-4-yl)-N- methyl-1,4,6,7-tetrahydro-5H- pyrazolo[4,3-c]pyridine-5- carboxamide |
| CPD- 052 (P- 065) | 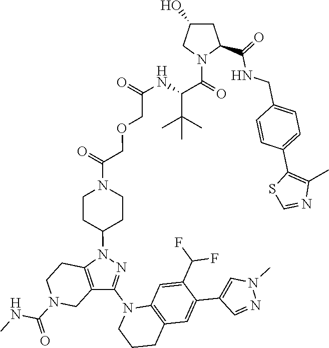 | 3-(7-(difluoromethyl)-6-(1-methyl- 1H-pyrazol-4-yl)-3,4- dihydroquinolin-1(2H)-yl)-1-(1-(3- (3-(((S)-1-((2S,4R)-4-hydroxy-2-((4- (4-methylthiazol-5- yl)benzyl)carbamoyl)pyrrolidin-1- yl)-3,3-dimethyl-1-oxobutan-2- yl)amino)-3- oxopropoxy)propanoyl)piperidin-4- yl)-N-methyl-1,4,6,7-tetrahydro-5H- pyrazolo[4,3-c]pyridine-5- carboxamide |
| CPD- 053 (P- 066) | 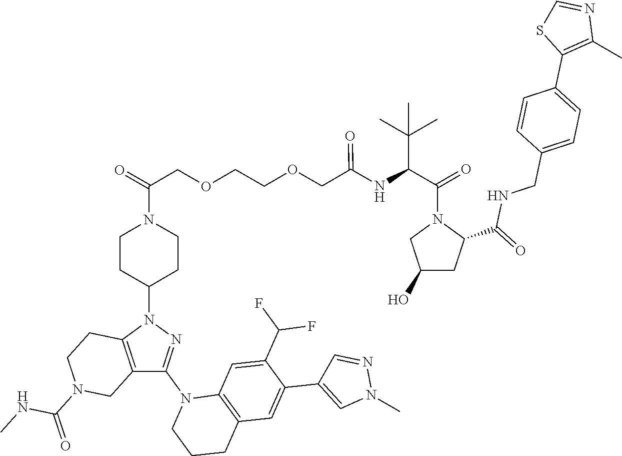 | 3-(7-(difluoromethyl)-6-(1-methyl- 1H-pyrazol-4-yl)-3,4- dihydroquinolin-1(2H)-yl)-1-(1-(2- (2-(2-(((S)-1-((2S,4R)-4-hydroxy-2- ((4-(4-methylthiazol-5- yl)benzyl)carbamoyl)pyrrolidin-1- yl)-3,3-dimethyl-1-oxobutan-2- yl)amino)-2- oxoethoxy)ethoxy)acetyl)piperidin- 4-yl)-N-methyl-1,4,6,7-tetrahydro- 5H-pyrazolo[4,3-c]pyridine-5- carboxamide |
| CPD- 054 (P- 067) | 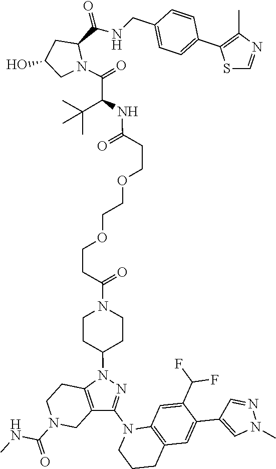 | 3-(7-(difluoromethyl)-6-(1-methyl- 1H-pyrazol-4-yl)-3,4- dihydroquinolin-1(2H)-yl)-1-(1-(3- (2-(3-(((S)-1-((2S,4R)-4-hydroxy-2- ((4-(4-methylthiazol-5- yl)benzyl)carbamoyl)pyrrolidin-1- yl)-3,3-dimethyl-1-oxobutan-2- yl)amino)-3- oxopropoxy)ethoxy)propanoyl) piperidin-4-yl)-N-methyl-1,4,6,7- tetrahydro-5H-pyrazolo[4,3- c]pyridine-5-carboxamide |
| CPD- 055 (P- 068) | 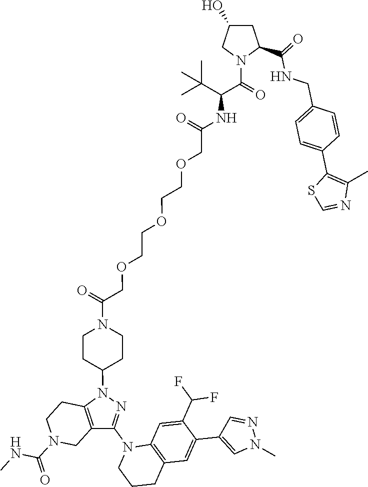 | 3-(7-(difluoromethyl)-6-(1-methyl- 1H-pyrazol-4-yl)-3,4- dihydroquinolin-1(2H)-yl)-1-(1- ((S)-13-((2S,4R)-4-hydroxy-2-((4- (4-methylthiazol-5- yl)benzyl)carbamoyl)pyrrolidine-1- carbonyl)-14,14-dimethyl-11-oxo- 3,6,9-trioxa-12- azapentadecanoyl)piperidin-4-yl)-N- methyl-1,4,6,7-tetrahydro-5H- pyrazolo[4,3-c]pyridine-5- carboxamide |
| CPD- 056 (P- 069) | 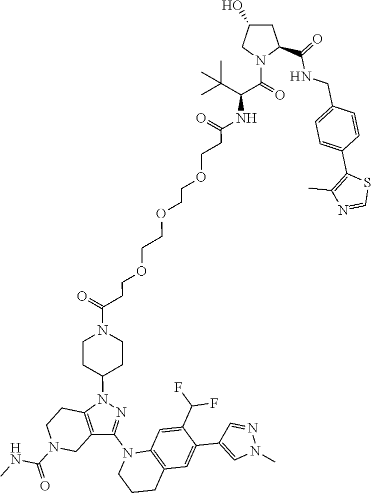 [SMILES: CNC(=O)N1CCc2c(c(N3CCCc4cc(-c5cnn(C)c5)c(C(F)F)cc43)nn2C2CCN(C(=O)CCOCCOCCOCCC(=O)N[C@H](C(=O)N3C[C@H](O)C[C@H]3C(=O)NCc3ccc(-c4scnc4C)cc3)C(C)(C)C)CC2)C1] | 3-(7-(difluoromethyl)-6-(1-methyl- 1H-pyrazol-4-yl)-3,4- dihydroquinolin-1(2H)-yl)-1-(1- ((S)-15-((2S,4R)-4-hydroxy-2-((4- (4-methylthiazol-5- yl)benzyl)carbamoyl)pyrrolidine-1- carbonyl)-16,16-dimethyl-13-oxo- 4,7,10-trioxa-14- azaheptadecanoyl)piperidin-4-yl)-N- methyl-1,4,6,7-tetrahydro-5H- pyrazolo[4,3-c]pyridine-5- carboxamide |
| CPD- 057 (P- 070) | 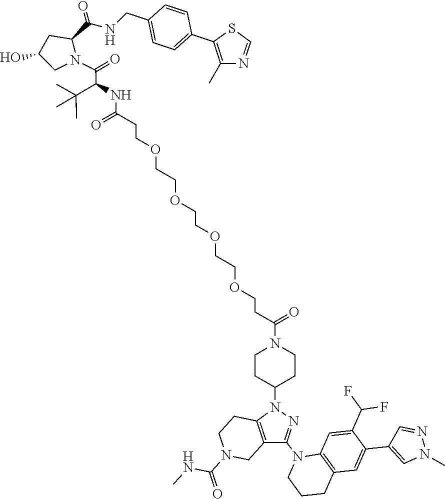 [SMILES: CNC(=O)N1CCc2c(c(N3CCCc4cc(-c5cnn(C)c5)c(C(F)F)cc43)nn2C2CCN(C(=O)CCOCCOCCOCCOCCC(=O)N[C@H](C(=O)N3C[C@H](O)C[C@H]3C(=O)NCc3ccc(-c4scnc4C)cc3)C(C)(C)C)CC2)C1] | 3-(7-(difluoromethyl)-6-(1-methyl- 1H-pyrazol-4-yl)-3,4- dihydroquinolin-1(2H)-yl)-1-(1- ((S)-18-((2S,4R)-4-hydroxy-2-((4- (4-methylthiazol-5- yl)benzyl)carbamoyl)pyrrolidine-1- carbonyl)-19,19-dimethyl-16-oxo- 4,7,10,13-tetraoxa-17- azaicosanoyl)piperidin-4-yl)-N- methyl-1,4,6,7-tetrahydro-5H- pyrazolo[4,3-c]pyridine-5- carboxamide |
| CPD- 058 (P- 071) | 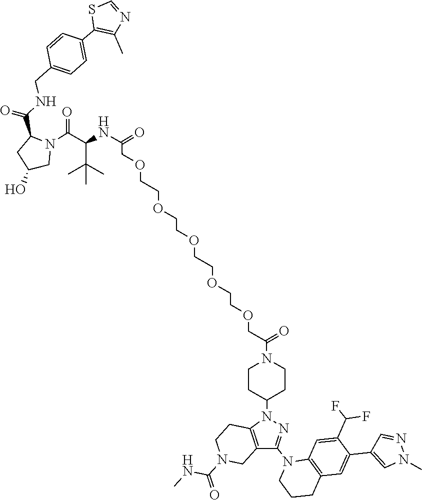 [SMILES: CNC(=O)N1CCc2c(c(N3CCCc4cc(-c5cnn(C)c5)c(C(F)F)cc43)nn2C2CCN(C(=O)COCCOCCOCCOCCOCC(=O)N[C@H](C(=O)N3C[C@H](O)C[C@H]3C(=O)NCc3ccc(-c4scnc4C)cc3)C(C)(C)C)CC2)C1] | 3-(7-(difluoromethyl)-6-(1-methyl- 1H-pyrazol-4-yl)-3,4- dihydroquinolin-1(2H)-yl)-1-(1- ((S)-19-((2S,4R)-4-hydroxy-2-((4- (4-methylthiazol-5- yl)benzyl)carbamoyl)pyrrolidine-1- carbonyl)-20,20-dimethyl-17-oxo- 3,6,9,12,15-pentaoxa-18- azahenicosanoyl)piperidin-4-yl)-N- methyl-1,4,6,7-tetrahydro-5H- pyrazolo[4,3-c]pyridine-5- carboxamide |
| CPD- 059 (P- 072) | 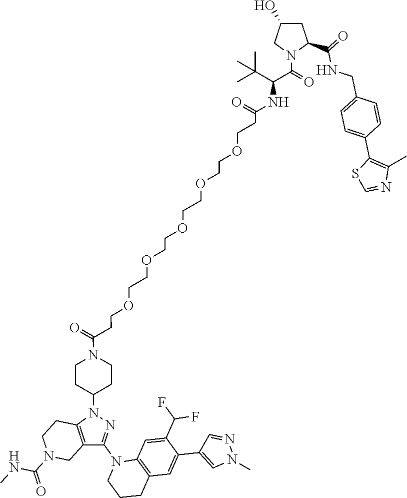 | 3-(7-(difluoromethyl)-6-(1-methyl- 1H-pyrazol-4-yl)-3,4- dihydroquinolin-1(2H)-yl)-1-(1- ((S)-21-((2S,4R)-4-hydroxy-2-((4- (4-methylthiazol-5- yl)benzyl)carbamoyl)pyrrolidine-1- carbonyl)-22,22-dimethyl-19-oxo- 4,7,10,13,16-pentaoxa-20- azatricosanoyl)piperidin-4-yl)-N- methyl-1,4,6,7-tetrahydro-5H- pyrazolo[4,3-c]pyridine-5- carboxamide |
| CPD- 060 (P- 056) | 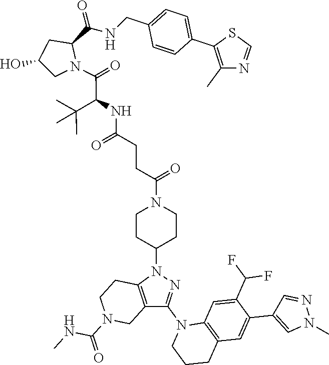 [SMILES: CNC(=O)N1CCc2c(c(N3CCCc4cc(-c5cnn(C)c5)c(C(F)F)cc43)nn2C2CCN(C(=O)CCC(=O)N[C@H](C(=O)N3C[C@H](O)C[C@H]3C(=O)NCc3ccc(-c4scnc4C)cc3)C(C)(C)C)CC2)C1] | 3-(7-(difluoromethyl)-6-(1-methyl- 1H-pyrazol-4-yl)-3,4- dihydroquinolin-1(2H)-yl)-1-(1-(4- (((S)-1-((2S,4R)-4-hydroxy-2-((4-(4- methylthiazol-5- yl)benzyl)carbamoyl)pyrrolidin-1- yl)-3,3-dimethyl-1-oxobutan-2- yl)amino)-4-oxobutanoyl)piperidin- 4-yl)-N-methyl-1,4,6,7-tetrahydro- 5H-pyrazolo[4,3-c]pyridine-5- carboxamide |
| CPD- 061 (P- 057) | 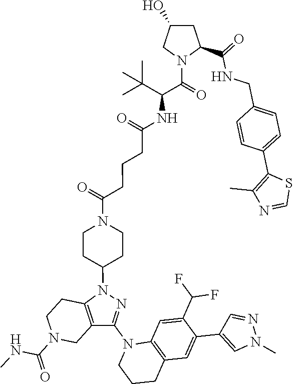 [SMILES: CNC(=O)N1CCc2c(c(N3CCCc4cc(-c5cnn(C)c5)c(C(F)F)cc43)nn2C2CCN(C(=O)CCCC(=O)N[C@H](C(=O)N3C[C@H](O)C[C@H]3C(=O)NCc3ccc(-c4scnc4C)cc3)C(C)(C)C)CC2)C1] | 3-(7-(difluoromethyl)-6-(1-methyl- 1H-pyrazol-4-yl)-3,4- dihydroquinolin-1(2H)-yl)-1-(1-(5- (((S)-1-((2S,4R)-4-hydroxy-2-((4-(4- methylthiazol-5- yl)benzyl)carbamoyl)pyrrolidin-1- yl)-3,3-dimethyl-1-oxobutan-2- yl)amino)-5- oxopentanoyl)piperidin-4-yl)-N- methyl-1,4,6,7-tetrahydro-5H- pyrazolo[4,3-c]pyridine-5- carboxamide |
| CPD- 062 (P- 058) | 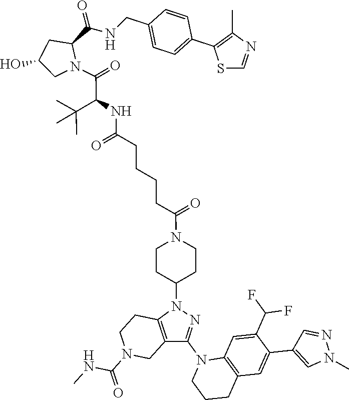 [SMILES: CNC(=O)N1CCc2c(c(N3CCCc4cc(-c5cnn(C)c5)c(C(F)F)cc43)nn2C2CCN(C(=O)CCCCC(=O)N[C@H](C(=O)N3C[C@H](O)C[C@H]3C(=O)NCc3ccc(-c4scnc4C)cc3)C(C)(C)C)CC2)C1] | 3-(7-(difluoromethyl)-6-(1-methyl- 1H-pyrazol-4-yl)-3,4- dihydroquinolin-1(2H)-yl)-1-(1-(6- (((S)-1-((2S,4R)-4-hydroxy-2-((4-(4- methylthiazol-5- yl)benzyl)carbamoyl)pyrrolidin-1- yl)-3,3-dimethyl-1-oxobutan-2- yl)amino)-6-oxohexanoyl)piperidin- 4-yl)-N-methyl-1,4,6,7-tetrahydro- 5H-pyrazolo[4,3-c]pyridine-5- carboxamide |
| CPD- 063 (P- 059) | 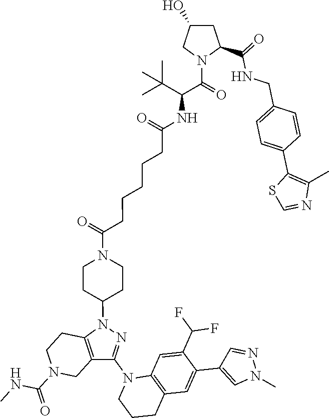 | 3-(7-(difluoromethyl)-6-(1-methyl- 1H-pyrazol-4-yl)-3,4- dihydroquinolin-1(2H)-yl)-1-(1-(7- (((S)-1-((2S,4R)-4-hydroxy-2-((4-(4- methylthiazol-5- yl)benzyl)carbamoyl)pyrrolidin-1- yl)-3,3-dimethyl-1-oxobutan-2- yl)amino)-7- oxoheptanoyl)piperidin-4-yl)-N- methyl-1,4,6,7-tetrahydro-5H- pyrazolo[4,3-c]pyridine-5- carboxamide |
| CPD- 064 (P- 060) | 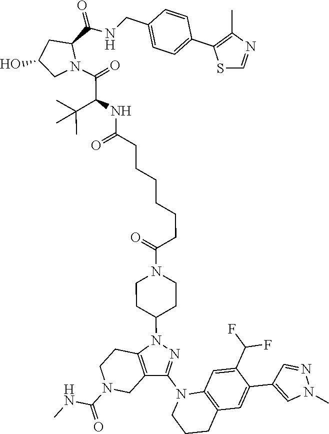 [SMILES: CNC(=O)N1CCc2c(c(N3CCCc4cc(-c5cnn(C)c5)c(C(F)F)cc43)nn2C2CCN(C(=O)CCCCCCC(=O)N[C@H](C(=O)N3C[C@H](O)C[C@H]3C(=O)NCc3ccc(-c4scnc4C)cc3)C(C)(C)C)CC2)C1] | 3-(7-(difluoromethyl)-6-(1-methyl- 1H-pyrazol-4-yl)-3,4- dihydroquinolin-1(2H)-yl)-1-(1-(8- (((S)-1-((2S,4R)-4-hydroxy-2-((4-(4- methylthiazol-5- yl)benzyl)carbamoyl)pyrrolidin-1- yl)-3,3-dimethyl-1-oxobutan-2- yl)amino)-8-oxooctanoyl)piperidin- 4-yl)-N-methyl-1,4,6,7-tetrahydro- 5H-pyrazolo[4,3-c]pyridine-5- carboxamide |
| CPD- 065 (P- 061) | 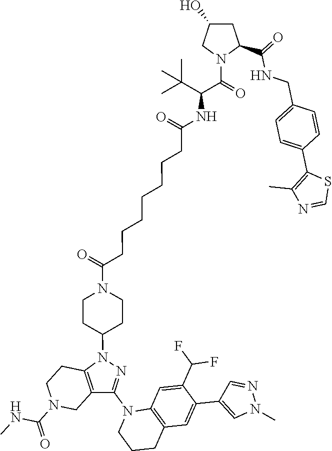 [SMILES: CNC(=O)N1CCc2c(c(N3CCCc4cc(-c5cnn(C)c5)c(C(F)F)cc43)nn2C2CCN(C(=O)CCCCCCCC(=O)N[C@H](C(=O)N3C[C@H](O)C[C@H]3C(=O)NCc3ccc(-c4scnc4C)cc3)C(C)(C)C)CC2)C1] | 3-(7-(difluoromethyl)-6-(1-methyl- 1H-pyrazol-4-yl)-3,4- dihydroquinolin-1(2H)-yl)-1-(1-(9- (((S)-1-((2S,4R)-4-hydroxy-2-((4-(4- methylthiazol-5- yl)benzyl)carbamoyl)pyrrolidin-1- yl)-3,3-dimethyl-1-oxobutan-2- yl)amino)-9-oxononanoyl)piperidin- 4-yl)-N-methyl-1,4,6,7-tetrahydro- 5H-pyrazolo[4,3-c]pyridine-5- carboxamide |
| CPD- 066 (P- 062) | 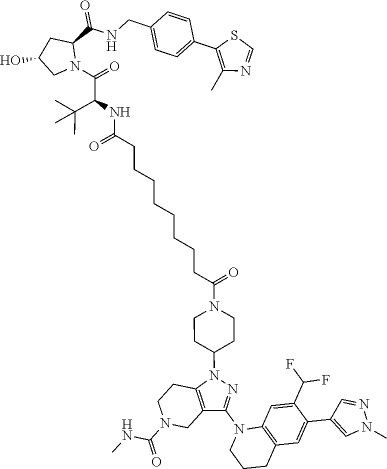 [SMILES: CNC(=O)N1CCc2c(c(N3CCCc4cc(-c5cnn(C)c5)c(C(F)F)cc43)nn2C2CCN(C(=O)CCCCCCCCC(=O)N[C@H](C(=O)N3C[C@H](O)C[C@H]3C(=O)NCc3ccc(-c4scnc4C)cc3)C(C)(C)C)CC2)C1] | 3-(7-(difluoromethyl)-6-(1-methyl- 1H-pyrazol-4-yl)-3,4- dihydroquinolin-1(2H)-yl)-1-(1-(10- (((S)-1-((2S,4R)-4-hydroxy-2-((4-(4- methylthiazol-5- yl)benzyl)carbamoyl)pyrrolidin-1- yl)-3,3-dimethyl-1-oxobutan-2- yl)amino)-10- oxodecanoyl)piperidin-4-yl)-N- methyl-1,4,6,7-tetrahydro-5H- pyrazolo[4,3-c]pyridine-5- carboxamide |
| CPD- 067 (P- 063) | 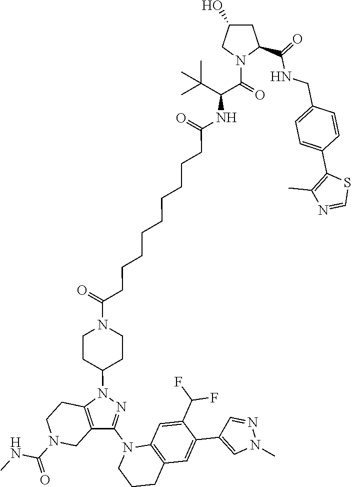 [SMILES: CNC(=O)N1CCc2c(c(N3CCCc4cc(-c5cnn(C)c5)c(C(F)F)cc43)nn2C2CCN(C(=O)CCCCCCCCCC(=O)N[C@H](C(=O)N3C[C@H](O)C[C@H]3C(=O)NCc3ccc(-c4scnc4C)cc3)C(C)(C)C)CC2)C1] | 3-(7-(difluoromethyl)-6-(1-methyl- 1H-pyrazol-4-yl)-3,4- dihydroquinolin-1(2H)-yl)-1-(1-(11- (((S)-1-((2S,4R)-4-hydroxy-2-((4-(4- methylthiazol-5- yl)benzyl)carbamoyl)pyrrolidin-1- yl)-3,3-dimethyl-1-oxobutan-2- yl)amino)-11- oxoundecanoyl)piperidin-4-yl)-N- methyl-1,4,6,7-tetrahydro-5H- pyrazolo[4,3-c]pyridine-5- carboxamide |
| CPD- 068 (P- 051) | 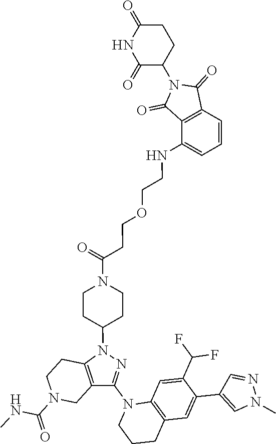 | 3-(7-(difluoromethyl)-6-(1-methyl- 1H-pyrazol-4-yl)-3,4- dihydroquinolin-1(2H)-yl)-1-(1-(3- (2-((2-(2,6-dioxopiperidin-3-yl)-1,3- dioxoisoindolin-4-yl) amino)ethoxy)propanoyl)piperidin- 4-yl)-N-methyl-1,4,6,7-tetrahydro- 5H-pyrazolo[4,3-c]pyridine-5- carboxamide |
| CPD- 069 (P- 052) | 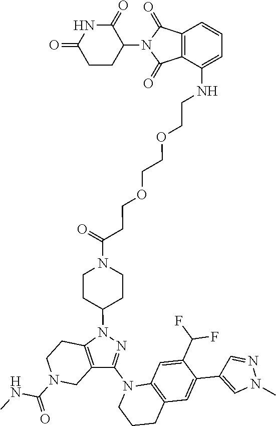 | 3-(7-(difluoromethyl)-6-(1-methyl- 1H-pyrazol-4-yl)-3,4- dihydroquinolin-1(2H)-yl)-1-(1-(3- (2-(2-((2-(2,6-dioxopiperidin-3-yl)- 1,3-dioxoisoindolin-4- yl)amino)ethoxy)ethoxy)propanoyl) piperidin-4-yl)-N-methyl-1,4,6,7- tetrahydro-5H-pyrazolo[4,3- c]pyridine-5-carboxamide |
| CPD- 070 (P- 053) | 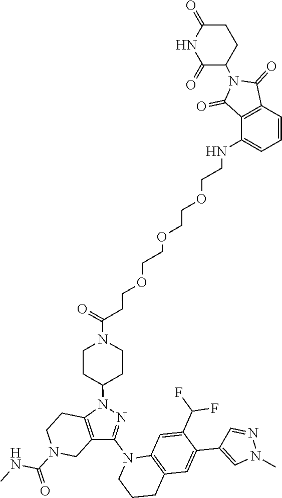 [SMILES: CNC(=O)N1CCc2c(c(N3CCCc4cc(-c5cnn(C)c5)c(C(F)F)cc43)nn2C2CCN(C(=O)CCOCCOCCOCCNc3cccc4c3C(=O)N(C3CCC(=O)NC3=O)C4=O)CC2)C1] | 3-(7-(difluoromethyl)-6-(1-methyl- 1H-pyrazol-4-yl)-3,4- dihydroquinolin-1(2H)-yl)-1-(1-(3- (2-(2-(2-((2-(2,6-dioxopiperidin-3- yl)-1,3-dioxoisoindolin-4- yl)amino)ethoxy)ethoxy)ethoxy) propanoyl)piperidin-4-yl)-N-methyl- 1,4,6,7-tetrahydro-5H-pyrazolo[4,3- c]pyridine-5-carboxamide |
| CPD- 071 (P- 054) | 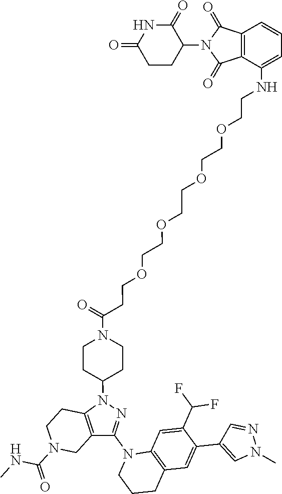 [SMILES: CNC(=O)N1CCc2c(c(N3CCCc4cc(-c5cnn(C)c5)c(C(F)F)cc43)nn2C2CCN(C(=O)CCOCCOCCOCCOCCNc3cccc4c3C(=O)N(C3CCC(=O)NC3=O)C4=O)CC2)C1] | 3-(7-(difluoromethyl)-6-(1-methyl- 1H-pyrazol-4-yl)-3,4- dihydroquinolin-1(2H)-yl)-1-(1-(1- ((2-(2,6-dioxopiperidin-3-yl)-1,3- dioxoisoindolin-4-yl)amino)- 3,6,9,12-tetraoxapentadecan-15- oyl)piperidin-4-yl)-N-methyl- 1,4,6,7-tetrahydro-5H-pyrazolo[4,3- c]pyridine-5-carboxamide |
| CPD- 072 (P- 055) | 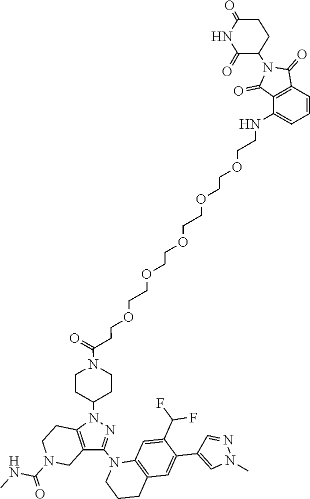 [SMILES: CNC(=O)N1CCc2c(c(N3CCCc4cc(-c5cnn(C)c5)c(C(F)F)cc43)nn2C2CCN(C(=O)CCOCCOCCOCCOCCOCCNc3cccc4c3C(=O)N(C3CCC(=O)NC3=O)C4=O)CC2)C1] | 3-(7-(difluoromethyl)-6-(1-methyl- 1H-pyrazol-4-yl)-3,4- dihydroquinolin-1(2H)-yl)-1-(1-(1- ((2-(2,6-dioxopiperidin-3-yl)-1,3- dioxoisoindolin-4-yl)amino)- 3,6,9,12,15-pentaoxaoctadecan-18- oyl)piperidin-4-yl)-N-methyl- 1,4,6,7-tetrahydro-5H-pyrazolo[4,3- c]pyridine-5-carboxamide |
| CPD- 073 (P- 073) | 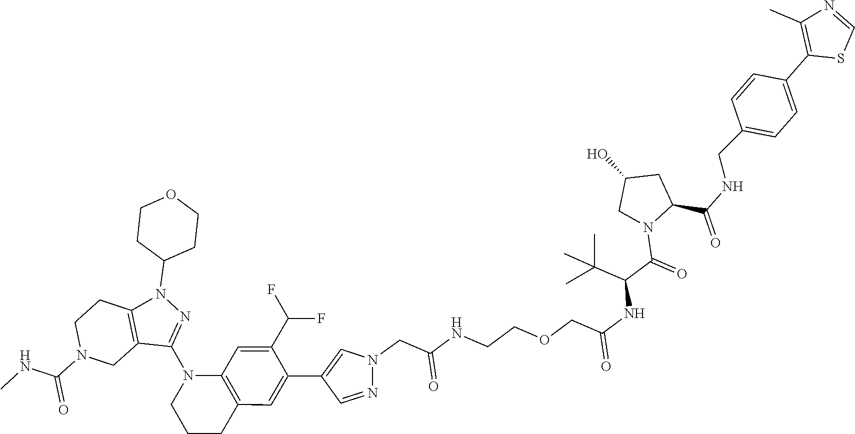 [SMILES: CNC(=O)N1CCc2c(c(N3CCCc4cc(-c5cnn(CC(=O)NCCOCC(=O)N[C@H](C(=O)N6C[C@H](O)C[C@H]6C(=O)NCc6ccc(-c7scnc7C)cc6)C(C)(C)C)c5)c(C(F)F)cc43)nn2C2CCOCC2)C1] | 3-(7-(difluoromethyl)-6-(1-(2-((2- (2-(((S)-1-((2S,4R)-4-hydroxy-2-((4- (4-methylthiazol-5- yl)benzyl)carbamoyl)pyrrolidin-1- yl)-3,3-dimethyl-1-oxobutan-2- yl)amino)-2- oxoethoxy)ethyl)amino)-2- oxoethyl)-1H-pyrazol-4-yl)-3,4- dihydroquinolin-1(2H)-yl)-N- methyl-1-(tetrahydro-2H-pyran-4- yl)-1,4,6,7-tetrahydro-5H- pyrazolo[4,3-c]pyridine-5- carboxamide |
| CPD- 074 (P- 074) | 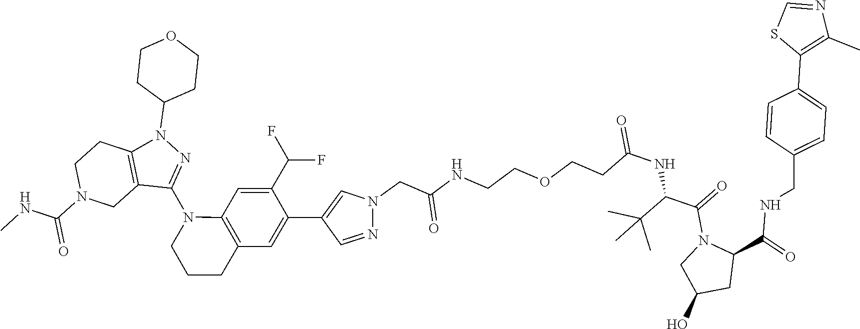 | 3-(7-(difluoromethyl)-6-(1-(2-((2- (3-(((S)-1-((2S,4R)-4-hydroxy-2-((4- (4-methylthiazol-5- yl)benzyl)carbamoyl)pyrrolidin-1- yl)-3,3-dimethyl-1-oxobutan-2- yl)amino)-3- oxopropoxy)ethyl)amino)-2- oxoethyl)-1H-pyrazol-4-yl)-3,4- dihydroquinolin-1(2H)-yl)-N- methyl-1-(tetrahydro-2H-pyran-4- yl)-1,4,6,7-tetrahydro-5H- pyrazolo[4,3-c]pyridine-5- carboxamide |
| CPD- 075 (P- 083) | 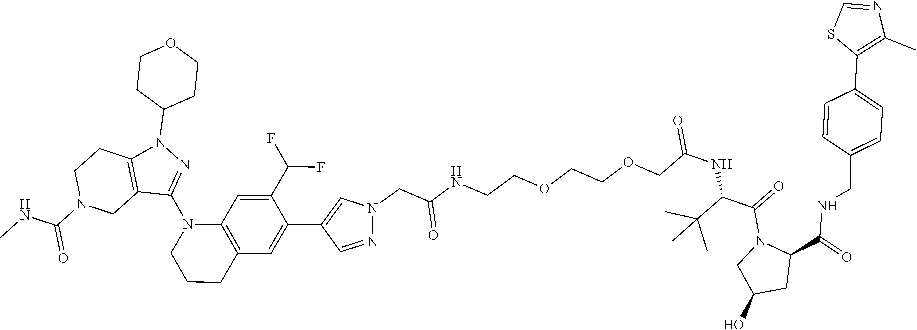 | 3-(7-(difluoromethyl)-6-(1-((S)-13- ((2S,4R)-4-hydroxy-2-((4-(4- methylthiazol-5- yl)benzyl)carbamoyl)pyrrolidine-1- carbonyl)-14,14-dimethyl-2,11- dioxo-6,9-dioxa-3,12- diazapentadecyl)-1H-pyrazol-4-yl)- 3,4-dihydroquinolin-1(2H)-yl)-N- methyl-1-(tetrahydro-2H-pyran-4- yl)-1,4,6,7-tetrahydro-5H- pyrazolo[4,3-c]pyridine-5- carboxamide |
| CPD- 076 (P- 075) | 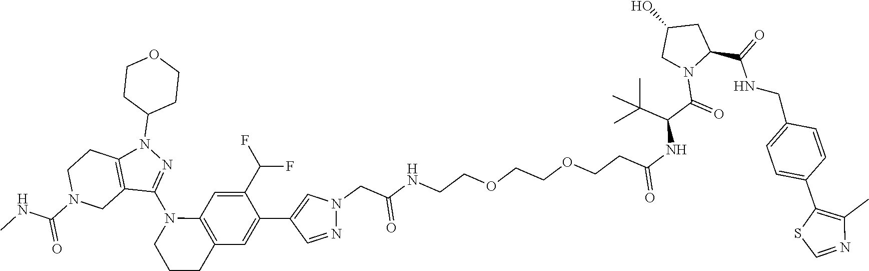 | 3-(7-(difluoromethyl)-6-(1-((S)-14- ((2S,4R)-4-hydroxy-2-((4-(4- methylthiazol-5- yl)benzyl)carbamoyl)pyrrolidine-1- carbonyl)-15,15-dimethyl-2,12- dioxo-6,9-dioxa-3,13- diazahexadecyl)-1H-pyrazol-4-yl)- 3,4-dihydroquinolin-1(2H)-yl)-N- methyl-1-(tetrahydro-2H-pyran-4- yl)-1,4,6,7-tetrahydro-5H- pyrazolo[4,3-c]pyridine-5- carboxamide |
| CPD- 077 (P- 076) | 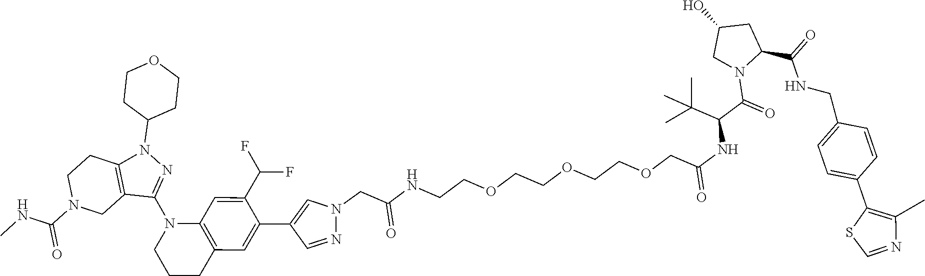 | 3-(7-(difluoromethyl)-6-(1-((S)-16- ((2S,4R)-4-hydroxy-2-((4-(4- methylthiazol-5- yl)benzyl)carbamoyl)pyrrolidine-1- carbonyl)-17,17-dimethyl-2,14- dioxo-6,9,12-trioxa-3,15- diazaoctadecyl)-1H-pyrazol-4-yl)- 3,4-dihydroquinolin-1(2H)-yl)-N- methyl-1-(tetrahydro-2H-pyran-4- yl)-1,4,6,7-tetrahydro-5H- pyrazolo[4,3-c]pyridine-5- carboxamide |
| CPD- 078 (P- 077) | 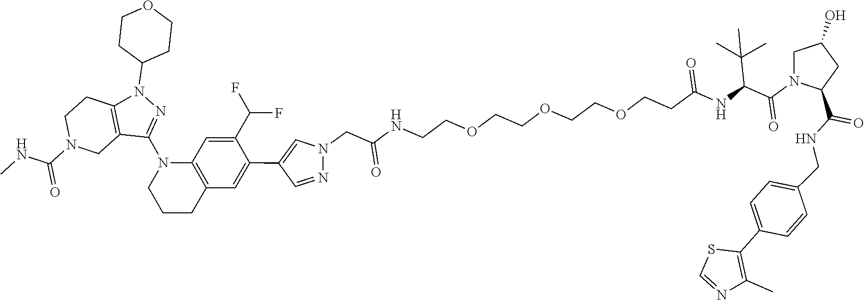 [SMILES: CNC(=O)N1CCc2c(c(N3CCCc4cc(-c5cnn(CC(=O)NCCOCCOCCOCCC(=O)N[C@H](C(=O)N6C[C@H](O)C[C@H]6C(=O)NCc6ccc(-c7scnc7C)cc6)C(C)(C)C)c5)c(C(F)F)cc43)nn2C2CCOCC2)C1] | 3-(7-(difluoromethyl)-6-(1-((S)-17- ((2S,4R)-4-hydroxy-2-((4-(4- methylthiazol-5- yl)benzyl)carbamoyl)pyrrolidine-1- carbonyl)-18,18-dimethyl-2,15- dioxo-6,9,12-trioxa-3,16- diazanonadecyl)-1H-pyrazol-4-yl)- 3,4-dihydroquinolin-1(2H)-yl)-N- methyl-1-(tetrahydro-2H-pyran-4- yl)-1,4,6,7-tetrahydro-5H- pyrazolo[4,3-c]pyridine-5- carboxamide |
| CPD- 079 (P- 078) | 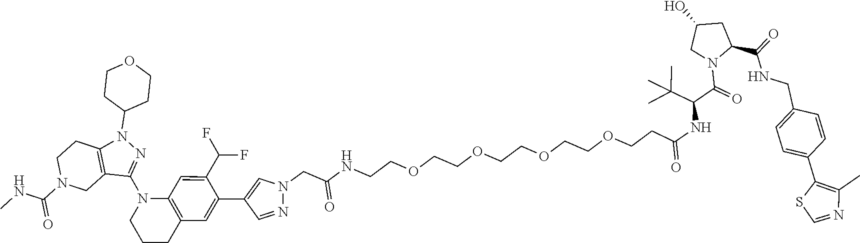 | 3-(7-(difluoromethyl)-6-(1-((S)-20- ((2S,4R)-4-hydroxy-2-((4-(4- methylthiazol-5- yl)benzyl)carbamoyl)pyrrolidine-1- carbonyl)-21,21-dimethyl-2,18- dioxo-6,9,12,15-tetraoxa-3,19- diazadocosyl)-1H-pyrazol-4-yl)-3,4- dihydroquinolin-1(2H)-yl)-N- methyl-1-(tetrahydro-2H-pyran-4- yl)-1,4,6,7-tetrahydro-5H- pyrazolo[4,3-c]pyridine-5- carboxamide |
| CPD- 080 (P- 082) | 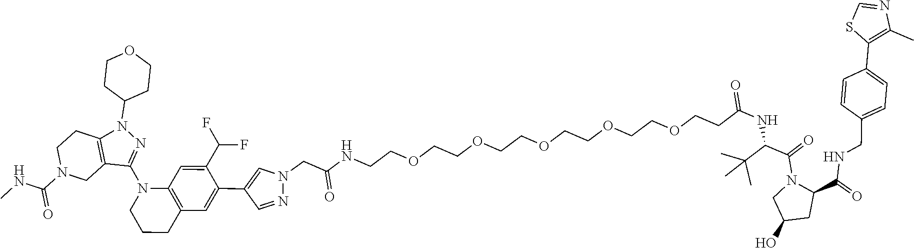 | 3-(7-(difluoromethyl)-6-(1-((S)-23- ((2S,4R)-4-hydroxy-2-((4-(4- methylthiazol-5- yl)benzyl)carbamoyl)pyrrolidine-1- carbonyl)-24,24-dimethyl-2,21- dioxo-6,9,12,15,18-pentaoxa-3,22- diazapentacosyl)-1H-pyrazol-4-yl)- 3,4-dihydroquinolin-1(2H)-yl)-N- methyl-1-(tetrahydro-2H-pyran-4- yl)-1,4,6,7-tetrahydro-5H- pyrazolo[4,3-c]pyridine-5- carboxamide |
| CPD- 081 (P- 079) | 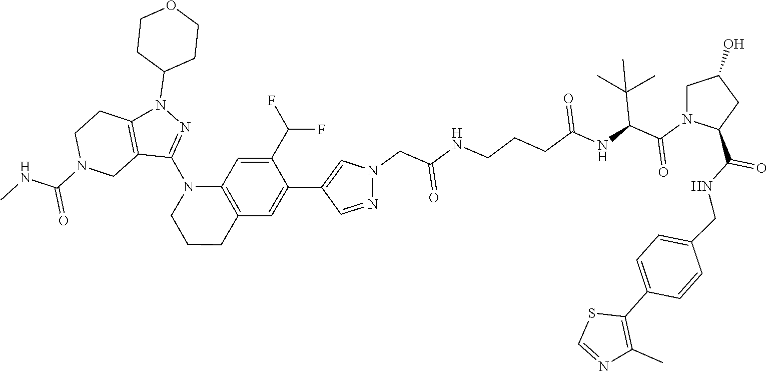 [SMILES: CNC(=O)N1CCc2c(c(N3CCCc4cc(-c5cnn(CC(=O)NCCCC(=O)N[C@H](C(=O)N6C[C@H](O)C[C@H]6C(=O)NCc6ccc(-c7scnc7C)cc6)C(C)(C)C)c5)c(C(F)F)cc43)nn2C2CCOCC2)C1] | 3-(7-(difluoromethyl)-6-(1-(2-((4- (((S)-1-((2S,4R)-4-hydroxy-2-((4-(4- methylthiazol-5- yl)benzyl)carbamoyl)pyrrolidin-1- yl)-3,3-dimethyl-1-oxobutan-2- yl)amino)-4-oxobutyl)amino)-2- oxoethyl)-1H-pyrazol-4-yl)-3,4- dihydroquinolin-1(2H)-yl)-N- methyl-1-(tetrahydro-2H-pyran-4- yl)-1,4,6,7-tetrahydro-5H- pyrazolo[4,3-c]pyridine-5- carboxamide |
| CPD- 082 (P- 080) | 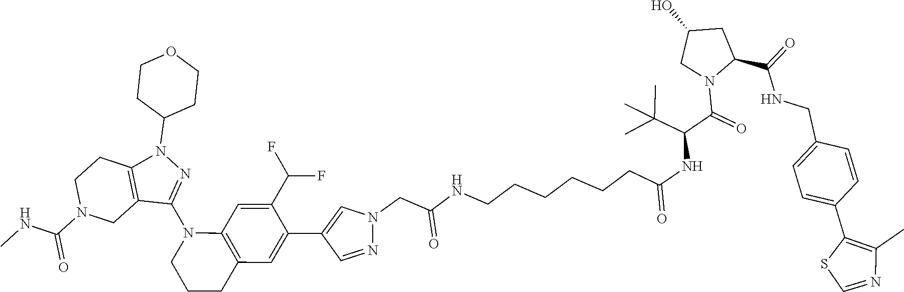 | 3-(7-(difluoromethyl)-6-(1-(2-((7- (((S)-1-((2S,4R)-4-hydroxy-2-((4-(4- methylthiazol-5- yl)benzyl)carbamoyl)pyrrolidin-1- yl)-3,3-dimethyl-1-oxobutan-2- yl)amino)-7-oxoheptyl)amino)-2- oxoethyl)-1H-pyrazol-4-yl)-3,4- dihydroquinolin-1(2H)-yl)-N- methyl-1-(tetrahydro-2H-pyran-4- yl)-1,4,6,7-tetrahydro-5H- pyrazolo[4,3-c]pyridine-5- carboxamide |
| CPD- 083 (P- 081) | 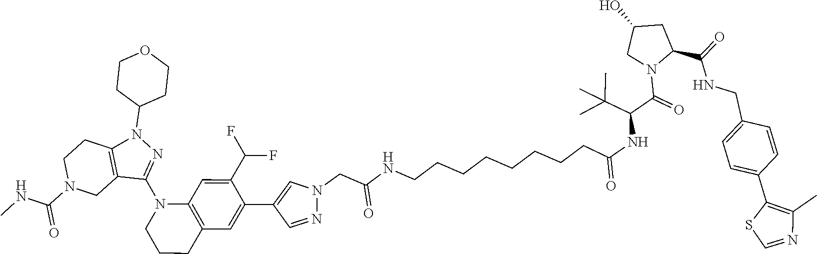 | 3-(7-(difluoromethyl)-6-(1-(2-((9- (((S)-1-((2S,4R)-4-hydroxy-2-((4-(4- methylthiazol-5- yl)benzyl)carbamoyl)pyrrolidin-1- yl)-3,3-dimethyl-1-oxobutan-2- yl)amino)-9-oxononyl)amino)-2- oxoethyl)-1H-pyrazol-4-yl)-3,4- dihydroquinolin-1(2H)-yl)-N- methyl-1-(tetrahydro-2H-pyran-4- yl)-1,4,6,7-tetrahydro-5H- pyrazolo[4,3-c]pyridine-5- carboxamide |
| P-084 | 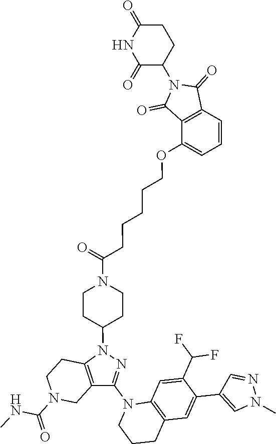 | 3-(7-(difluoromethyl)-6-(1-methyl- 1H-pyrazol-4-yl)-3,4- dihydroquinolin-1(2H)-yl)-1-(1-(6- ((2-(2,6-dioxopiperidin-3-yl)-1,3- dioxoisoindolin-4- yl)oxy)hexanoyl)piperidin-4-yl)-N- methyl-1,4,6,7-tetrahydro-5H- pyrazolo[4,3-c]pyridine-5- carboxamide |
| P-085 | 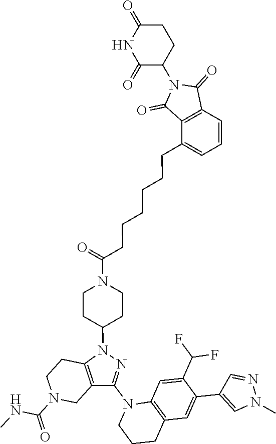 | 3-(7-(difluoromethyl)-6-(1-methyl- 1H-pyrazol-4-yl)-3,4- dihydroquinolin-1(2H)-yl)-1-(1-(7- (2-(2,6-dioxopiperidin-3-yl)-1,3- dioxoisoindolin-4- yl)heptanoyl)piperidin-4-yl)-N- methyl-1,4,6,7-tetrahydro-5H- pyrazolo[4,3-c]pyridine-5- carboxamide |
| P-086 | 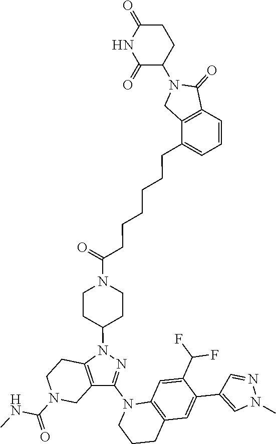 | 3-(7-(difluoromethyl)-6-(1-methyl- 1H-pyrazol-4-yl)-3,4- dihydroquinolin-1(2H)-yl)-1-(1-(7- (2-(2,6-dioxopiperidin-3-yl)-1- oxoisoindolin-4- yl)heptanoyl)piperidin-4-yl)-N- methyl-1,4,6,7-tetrahydro-5H- pyrazolo[4,3-c]pyridine-5- carboxamide |
| P-087 | 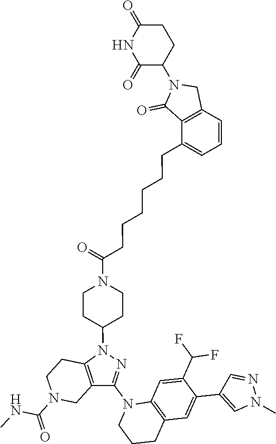 | 3-(7-(difluoromethyl)-6-(1-methyl- 1H-pyrazol-4-yl)-3,4- dihydroquinolin-1(2H)-yl)-1-(1-(7- (2-(2,6-dioxopiperidin-3-yl)-3- oxoisoindolin-4- yl)heptanoyl)piperidin-4-yl)-N- methyl-1,4,6,7-tetrahydro-5H- pyrazolo[4,3-c]pyridine-5- carboxamide |
| P-088 | 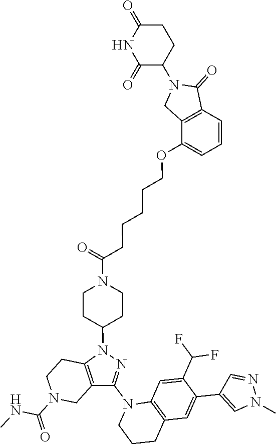 | 3-(7-(difluoromethyl)-6-(1-methyl- 1H-pyrazol-4-yl)-3,4- dihydroquinolin-1(2H)-yl)-1-(1-(6- ((2-(2,6-dioxopiperidin-3-yl)-1- oxoisoindolin-4- yl)oxy)hexanoyl)piperidin-4-yl)-N- methyl-1,4,6,7-tetrahydro-5H- pyrazolo[4,3-c]pyridine-5- carboxamide |
| P-089 | 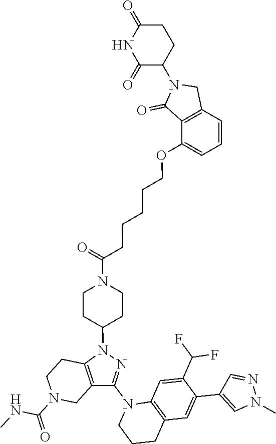 | 3-(7-(difluoromethyl)-6-(1-methyl- 1H-pyrazol-4-yl)-3,4- dihydroquinolin-1(2H)-yl)-1-(1-(6- ((2-(2,6-dioxopiperidin-3-yl)-3- oxoisoindolin-4- yl)oxy)hexanoyl)piperidin-4-yl)-N- methyl-1,4,6,7-tetrahydro-5H- pyrazolo[4,3-c]pyridine-5- carboxamide |
| P-090 | 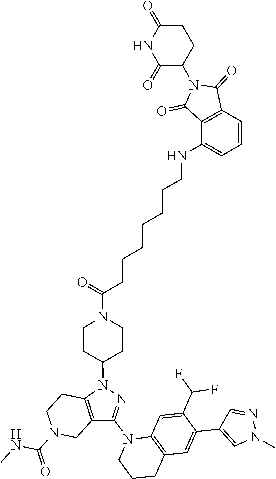 | 3-(7-(difluoromethyl)-6-(1-methyl- 1H-pyrazol-4-yl)-3,4- dihydroquinolin-1(2H)-yl)-1-(1-(6- ((2-(2,6-dioxopiperidin-3-yl)-1,3- dioxoisoindolin-4- yl)amino)hexyl)piperidin-4-yl)-N- methyl-1,4,6,7-tetrahydro-5H- pyrazolo[4,3-c]pyridine-5- carboxamide |
| P-091 | 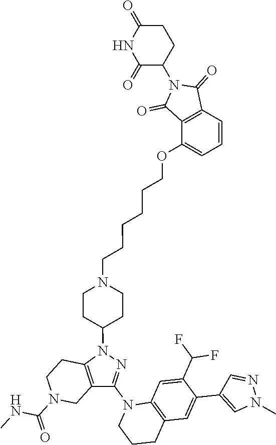 | 3-(7-(difluoromethyl)-6-(1-methyl- 1H-pyrazol-4-yl)-3,4- dihydroquinolin-1(2H)-yl)-1-(1-(6- ((2-(2,6-dioxopiperidin-3-yl)-1,3- dioxoisoindolin-4- yl)oxy)hexyl)piperidin-4-yl)-N- methyl-1,4,6,7-tetrahydro-5H- pyrazolo[4,3-c]pyridine-5- carboxamide |
| P-092 | 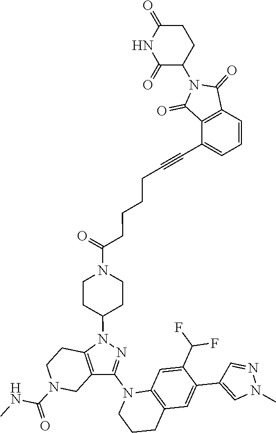 | 3-(7-(difluoromethyl)-6-(1-methyl- 1H-pyrazol-4-yl)-3,4- dihydroquinolin-1(2H)-yl)-1-(1-(7- (2-(2,6-dioxopiperidin-3-yl)-1,3- dioxoisoindolin-4-yl)hept-6- ynoyl)piperidin-4-yl)-N-methyl- 1,4,6,7-tetrahydro-5H-pyrazolo[4,3- c]pyridine-5-carboxamide |
| P-093 | 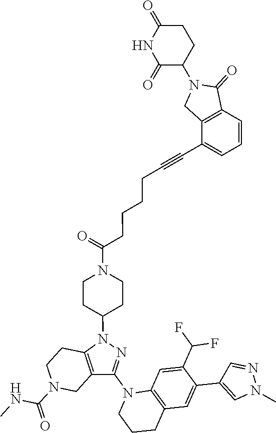 | 3-(7-(difluoromethyl)-6-(1-methyl- 1H-pyrazol-4-yl)-3,4- dihydroquinolin-1(2H)-yl)-1-(1-(7- (2-(2,6-dioxopiperidin-3-yl)-1- oxoisoindolin-4-yl)hept-6- ynoyl)piperidin-4-yl)-N-methyl- 1,4,6,7-tetrahydro-5H-pyrazolo[4,3- c]pyridine-5-carboxamide |
| P-094 | 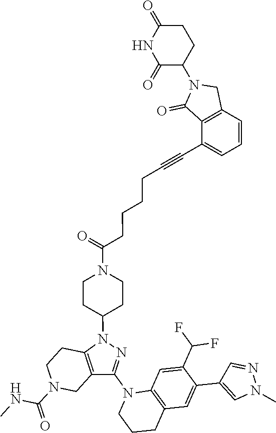 | 3-(7-(difluoromethyl)-6-(1-methyl- 1H-pyrazol-4-yl)-3,4- dihydroquinolin-1(2H)-yl)-1-(1-(7- (2-(2,6-dioxopiperidin-3-yl)-3- oxoisoindolin-4-yl)hept-6- ynoyl)piperidin-4-yl)-N-methyl- 1,4,6,7-tetrahydro-5H-pyrazolo[4,3- c]pyridine-5-carboxamide |
| P-095 | 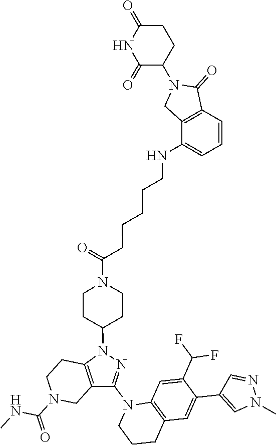 | 3-(7-(difluoromethyl)-6-(1-methyl- 1H-pyrazol-4-yl)-3,4- dihydroquinolin-1(2H)-yl)-1-(1-(6- ((2-(2,6-dioxopiperidin-3-yl)-1- oxoisoindolin-4- yl)amino)hexanoyl)piperidin-4-yl)- N-methyl-1,4,6,7-tetrahydro-5H- pyrazolo[4,3-c]pyridine-5- carboxamide |
| P-096 | 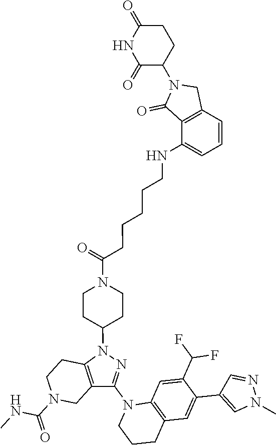 | 3-(7-(difluoromethyl)-6-(1-methyl- 1H-pyrazol-4-yl)-3,4- dihydroquinolin-1(2H)-yl)-1-(1-(6- ((2-(2,6-dioxopiperidin-3-yl)-3- oxoisoindolin-4- yl)amino)hexanoyl)piperidin-4-yl)- N-methyl-1,4,6,7-tetrahydro-5H- pyrazolo[4,3-c]pyridine-5- carboxamide |
| P-097 | 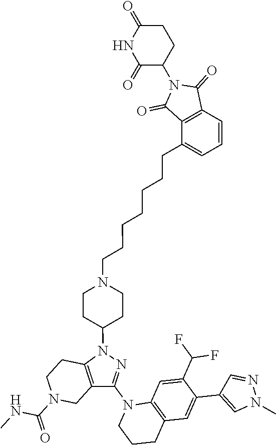 | 3-(7-(difluoromethyl)-6-(1-methyl- 1H-pyrazol-4-yl)-3,4- dihydroquinolin-1(2H)-yl)-1-(1-(7- (2-(2,6-dioxopiperidin-3-yl)-1,3- dioxoisoindolin-4- yl)heptyl)piperidin-4-yl)-N-methyl- 1,4,6,7-tetrahydro-5H-pyrazolo[4,3- c]pyridine-5-carboxamide |
| P-098 | 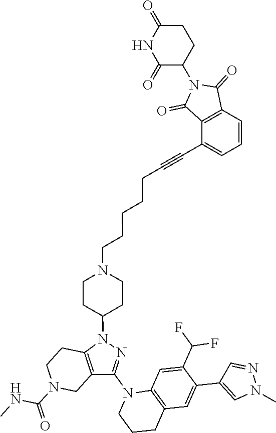 | 3-(7-(difluoromethyl)-6-(1-methyl- 1H-pyrazol-4-yl)-3,4- dihydroquinolin-1(2H)-yl)-1-(1-(7- (2-(2,6-dioxopiperidin-3-yl)-1,3- dioxoisoindolin-4-yl)hept-6-yn-1- yl)piperidin-4-yl)-N-methyl-1,4,6,7- tetrahydro-5H-pyrazolo[4,3- c]pyridine-5-carboxamide |
| P-100 | 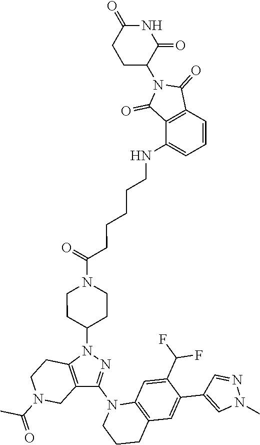 [SMILES: CC(=O)N1CCc2c(c(N3CCCc4cc(-c5cnn(C)c5)c(C(F)F)cc43)nn2C2CCN(C(=O)CCCCCNc3cccc4c3C(=O)N(C3CCC(=O)NC3=O)C4=O)CC2)C1] | 4-((6-(4-(5-acetyl-3-(7- (difluoromethyl)-6-(1-methyl-1H- pyrazol-4-yl)-3,4-dihydroquinolin- 1(2H)-yl)-4,5,6,7-tetrahydro-1H- pyrazolo[4,3-c]pyridin-1- yl)piperidin-1-yl)-6- oxohexyl)amino)-2-(2,6- dioxopiperidin-3-yl)isoindoline-1,3- dione |
| P-101 | 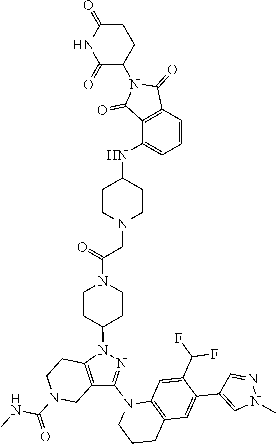 | 3-(7-(difluoromethyl)-6-(1-methyl- 1H-pyrazol-4-yl)-3,4- dihydroquinolin-1(2H)-yl)-1-(1-(2- (4-((2-(2,6-dioxopiperidin-3-yl)-1,3- dioxoisoindolin-4- yl)amino)piperidin-1- yl)acetyl)piperidin-4-yl)-N-methyl- 1,4,6,7-tetrahydro-5H-pyrazolo[4,3- c]pyridine-5-carboxamide |
| P-102 | 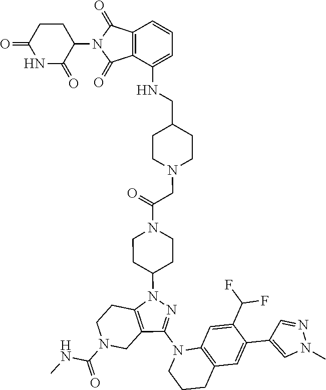 [SMILES: CNC(=O)N1CCc2c(c(N3CCCc4cc(-c5cnn(C)c5)c(C(F)F)cc43)nn2C2CCN(C(=O)CN3CCC(CNc4cccc5c4C(=O)N(C4CCC(=O)NC4=O)C5=O)CC3)CC2)C1] | 3-(7-(difluoromethyl)-6-(1-methyl- 1H-pyrazol-4-yl)-3,4- dihydroquinolin-1(2H)-yl)-1-(1-(2- (4-(((2-(2,6-dioxopiperidin-3-yl)- 1,3-dioxoisoindolin-4- yl)amino)methyl)piperidin-1- yl)acetyl)piperidin-4-yl)-N-methyl- 1,4,6,7-tetrahydro-5H-pyrazolo[4,3- c]pyridine-5-carboxamide |
| P-103 | 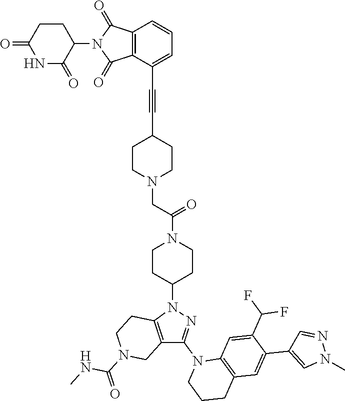 [SMILES: CNC(=O)N1CCc2c(c(N3CCCc4cc(-c5cnn(C)c5)c(C(F)F)cc43)nn2C2CCN(C(=O)CN3CCC(C#Cc4cccc5c4C(=O)N(C4CCC(=O)NC4=O)C5=O)CC3)CC2)C1] | 3-(7-(difluoromethyl)-6-(1-methyl- 1H-pyrazol-4-yl)-3,4- dihydroquinolin-1(2H)-yl)-1-(1-(2- (4-((2-(2,6-dioxopiperidin-3-yl)-1,3- dioxoisoindolin-4- yl)ethynyl)piperidin-1- yl)acetyl)piperidin-4-yl)-N-methyl- 1,4,6,7-tetrahydro-5H-pyrazolo[4,3- c]pyridine-5-carboxamide |
| P-104 | 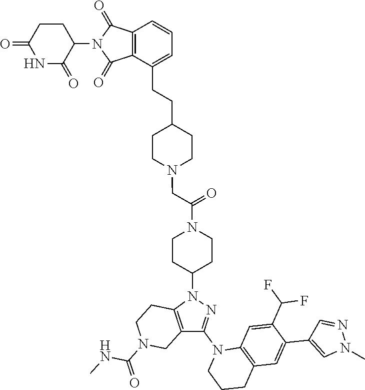 [SMILES: CNC(=O)N1CCc2c(c(N3CCCc4cc(-c5cnn(C)c5)c(C(F)F)cc43)nn2C2CCN(C(=O)CN3CCC(CCc4cccc5c4C(=O)N(C4CCC(=O)NC4=O)C5=O)CC3)CC2)C1] | 3-(7-(difluoromethyl)-6-(1-methyl- 1H-pyrazol-4-yl)-3,4- dihydroquinolin-1(2H)-yl)-1-(1-(2- (4-(2-(2-(2,6-dioxopiperidin-3-yl)- 1,3-dioxoisoindolin-4- yl)ethyl)piperidin-1- yl)acetyl)piperidin-4-yl)-N-methyl- 1,4,6,7-tetrahydro-5H-pyrazolo[4,3- c]pyridine-5-carboxamide |
| P-105 | 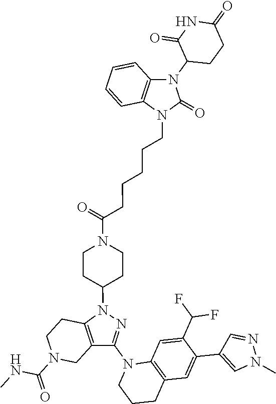 | 3-(7-(difluoromethyl)-6-(1-methyl- 1H-pyrazol-4-yl)-3,4- dihydroquinolin-1(2H)-yl)-1-(1-(6- (3-(2,6-dioxopiperidin-3-yl)-2-oxo- 2,3-dihydro-1H-benzo[d]imidazol- 1-yl)hexanoyl)piperidin-4-yl)-N- methyl-1,4,6,7-tetrahydro-5H- pyrazolo[4,3-c]pyridine-5- carboxamide |
| P-106 | 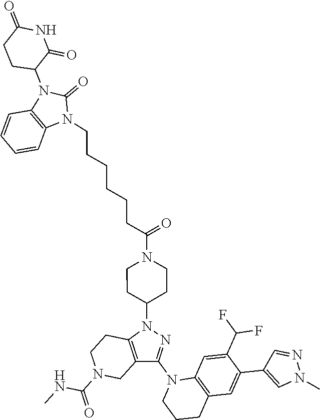 [SMILES: CNC(=O)N1CCc2c(c(N3CCCc4cc(-c5cnn(C)c5)c(C(F)F)cc43)nn2C2CCN(C(=O)CCCCCCn3c(=O)n(C4CCC(=O)NC4=O)c4ccccc43)CC2)C1] | 3-(7-(difluoromethyl)-6-(1-methyl- 1H-pyrazol-4-yl)-3,4- dihydroquinolin-1(2H)-yl)-1-(1-(7- (3-(2,6-dioxopiperidin-3-yl)-2-oxo- 2,3-dihydro-1H-benzo[d]imidazol- 1-yl)heptanoyl)piperidin-4-yl)-N- methyl-1,4,6,7-tetrahydro-5H- pyrazolo[4,3-c]pyridine-5- carboxamide |
| P-107 | 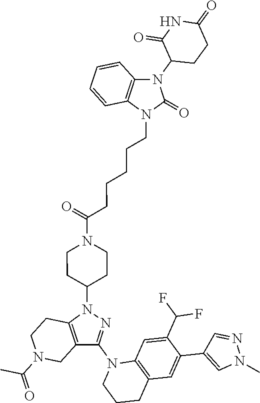 [SMILES: CC(=O)N1CCc2c(c(N3CCCc4cc(-c5cnn(C)c5)c(C(F)F)cc43)nn2C2CCN(C(=O)CCCCCn3c(=O)n(C4CCC(=O)NC4=O)c4ccccc43)CC2)C1] | 3-(3-(6-(4-(5-acetyl-3-(7- (difluoromethyl)-6-(1-methyl-1H- pyrazol-4-yl)-3,4-dihydroquinolin- 1(2H)-yl)-4,5,6,7-tetrahydro-1H- pyrazolo[4,3-c]pyridin-1- yl)piperidin-1-yl)-6-oxohexyl)-2- oxo-2,3-dihydro-1H- benzo[d]imidazol-1-yl)piperidine- 2,6-dione |
| P-108 | 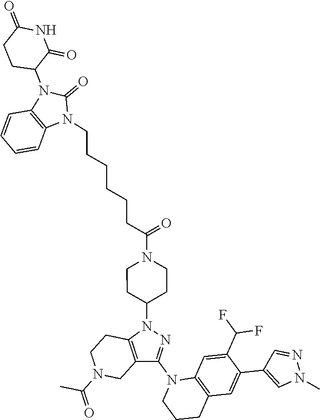 [SMILES: CC(=O)N1CCc2c(c(N3CCCc4cc(-c5cnn(C)c5)c(C(F)F)cc43)nn2C2CCN(C(=O)CCCCCCn3c(=O)n(C4CCC(=O)NC4=O)c4ccccc43)CC2)C1] | 3-(3-(7-(4-(5-acetyl-3-(7- (difluoromethyl)-6-(1-methyl-1H- pyrazol-4-yl)-3,4-dihydroquinolin- 1(2H)-yl)-4,5,6,7-tetrahydro-1H- pyrazolo[4,3-c]pyridin-1- yl)piperidin-1-yl)-7-oxoheptyl)-2- oxo-2,3-dihydro-1H- benzo[d]imidazol-1-yl)piperidine- 2,6-dione |
| P-109 | 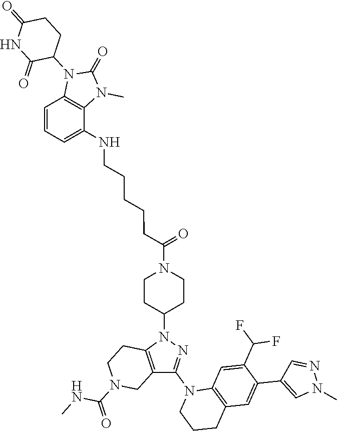 [SMILES: CNC(=O)N1CCc2c(c(N3CCCc4cc(-c5cnn(C)c5)c(C(F)F)cc43)nn2C2CCN(C(=O)CCCCCNc3cccc4c3n(C)c(=O)n4C3CCC(=O)NC3=O)CC2)C1] | 3-(7-(difluoromethyl)-6-(1-methyl- 1H-pyrazol-4-yl)-3,4- dihydroquinolin-1(2H)-yl)-1-(1-(6- ((1-(2,6-dioxopiperidin-3-yl)-3- methyl-2-oxo-2,3-dihydro-1H- benzo[d]imidazol-4- yl)amino)hexanoyl)piperidin-4-yl)- N-methyl-1,4,6,7-tetrahydro-5H- pyrazolo[4,3-c]pyridine-5- carboxamide |
| P-110 | 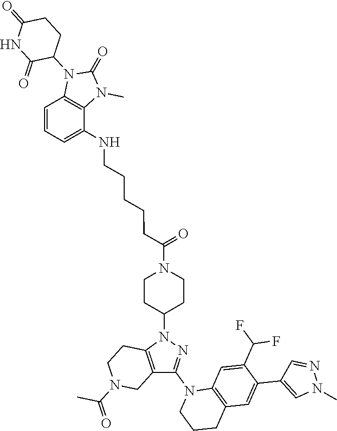 [SMILES: CC(=O)N1CCc2c(c(N3CCCc4cc(-c5cnn(C)c5)c(C(F)F)cc43)nn2C2CCN(C(=O)CCCCCNc3cccc4c3n(C)c(=O)n4C3CCC(=O)NC3=O)CC2)C1] | 3-(4-((6-(4-(5-acetyl-3-(7- (difluoromethyl)-6-(1-methyl-1H- pyrazol-4-yl)-3,4-dihydroquinolin- 1(2H)-yl)-4,5,6,7-tetrahydro-1H- pyrazolo[4,3-c]pyridin-1- yl)piperidin-1-yl)-6- oxohexyl)amino)-3-methyl-2-oxo- 2,3-dihydro-1H-benzo[d]imidazol- 1-yl)piperidine-2,6-dione |
| P-111 | 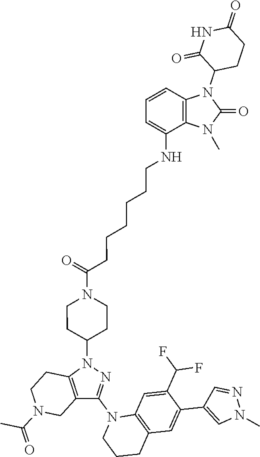 [SMILES: CC(=O)N1CCc2c(c(N3CCCc4cc(-c5cnn(C)c5)c(C(F)F)cc43)nn2C2CCN(C(=O)CCCCCCNc3cccc4c3n(C)c(=O)n4C3CCC(=O)NC3=O)CC2)C1] | 3-(4-((7-(4-(5-acetyl-3-(7- (difluoromethyl)-6-(1-methyl-1H- pyrazol-4-yl)-3,4-dihydroquinolin- 1(2H)-yl)-4,5,6,7-tetrahydro-1H- pyrazolo[4,3-c]pyridin-1- yl)piperidin-1-yl)-7- oxoheptyl)amino)-3-methyl-2-oxo- 2,3-dihydro-1H-benzo[d]imidazol- 1-yl)piperidine-2,6-dione |
| P-112 | 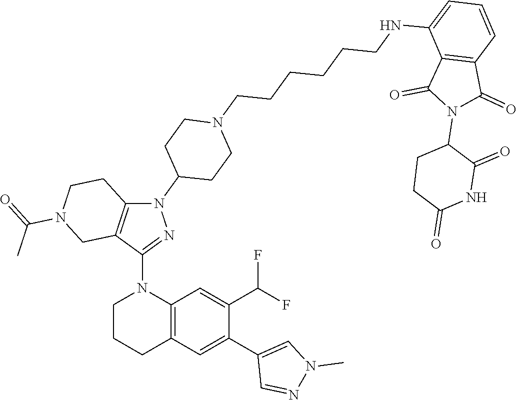 [SMILES: CC(=O)N1CCc2c(c(N3CCCc4cc(-c5cnn(C)c5)c(C(F)F)cc43)nn2C2CCN(CCCCCCNc3cccc4c3C(=O)N(C3CCC(=O)NC3=O)C4=O)CC2)C1] | 4-((6-(4-(5-acetyl-3-(7- (difluoromethyl)-6-(1-methyl-1H- pyrazol-4-yl)-3,4-dihydroquinolin- 1(2H)-yl)-4,5,6,7-tetrahydro-1H- pyrazolo[4,3-c]pyridin-1- yl)piperidin-1-yl)hexyl)amino)-2- (2,6-dioxopiperidin-3- yl)isoindoline-1,3-dione |
| P-113 | 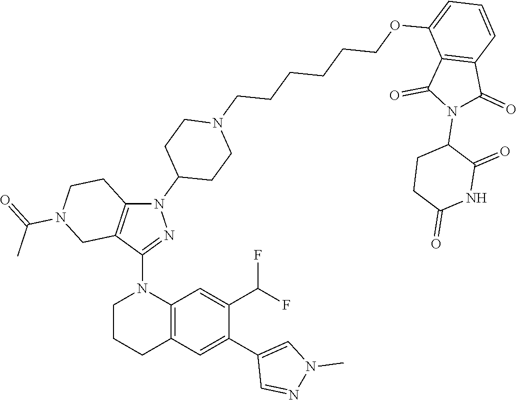 [SMILES: CC(=O)N1CCc2c(c(N3CCCc4cc(-c5cnn(C)c5)c(C(F)F)cc43)nn2C2CCN(CCCCCCOc3cccc4c3C(=O)N(C3CCC(=O)NC3=O)C4=O)CC2)C1] | 4-((6-(4-(5-acetyl-3-(7- (difluoromethyl)-6-(1-methyl-1H- pyrazol-4-yl)-3,4-dihydroquinolin- 1(2H)-yl)-4,5,6,7-tetrahydro-1H- pyrazolo[4,3-c]pyridin-1- yl)piperidin-1-yl)hexyl)oxy)-2-(2,6- dioxopiperidin-3-yl)isoindoline-1,3- dione |
| P-114 | 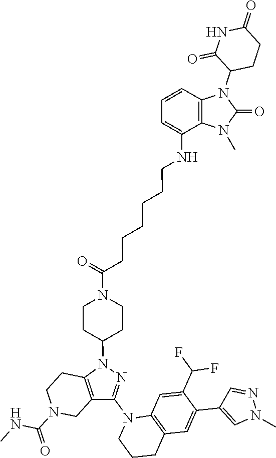 | 3-(7-(difluoromethyl)-6-(1-methyl- 1H-pyrazol-4-yl)-3,4- dihydroquinolin-1(2H)-yl)-1-(1-(7- ((1-(2,6-dioxopiperidin-3-yl)-3- methyl-2-oxo-2,3-dihydro-1H- benzo[d]imidazol-4- yl)amino)heptanoyl)piperidin-4-yl)- N-methyl-1,4,6,7-tetrahydro-5H- pyrazolo[4,3-c]pyridine-5- carboxamide |
| P-115 | 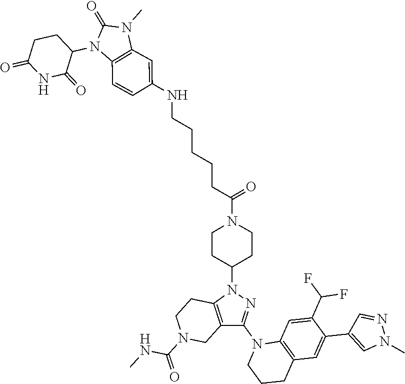 [SMILES: CNC(=O)N1CCc2c(c(N3CCCc4cc(-c5cnn(C)c5)c(C(F)F)cc43)nn2C2CCN(C(=O)CCCCCNc3ccc4c(c3)n(C)c(=O)n4C3CCC(=O)NC3=O)CC2)C1] | 3-(7-(difluoromethyl)-6-(1-methyl- 1H-pyrazol-4-yl)-3,4- dihydroquinolin-1(2H)-yl)-1-(1-(6- ((1-(2,6-dioxopiperidin-3-yl)-3- methyl-2-oxo-2,3-dihydro-1H- benzo[d]imidazol-5- yl)amino)hexanoyl)piperidin-4-yl)- N-methyl-1,4,6,7-tetrahydro-5H- pyrazolo[4,3-c]pyridine-5- carboxamide |
| P-116 | 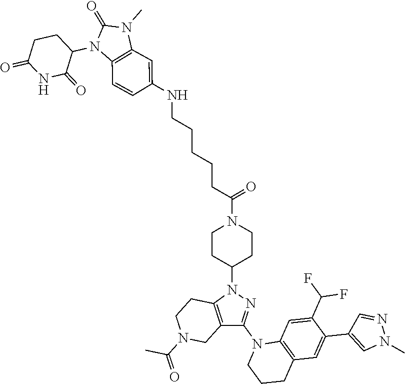 [SMILES: CC(=O)N1CCc2c(c(N3CCCc4cc(-c5cnn(C)c5)c(C(F)F)cc43)nn2C2CCN(C(=O)CCCCCNc3ccc4c(c3)n(C)c(=O)n4C3CCC(=O)NC3=O)CC2)C1] | 3-(5-((6-(4-(5-acetyl-3-(7- (difluoromethyl)-6-(1-methyl-1H- pyrazol-4-yl)-3,4-dihydroquinolin- 1(2H)-yl)-4,5,6,7-tetrahydro-1H- pyrazolo[4,3-c]pyridin-1- yl)piperidin-1-yl)-6- oxohexyl)amino)-3-methyl-2-oxo- 2,3-dihydro-1H-benzo[d]imidazol- 1-yl)piperidine-2,6-dione |
| P-117 | 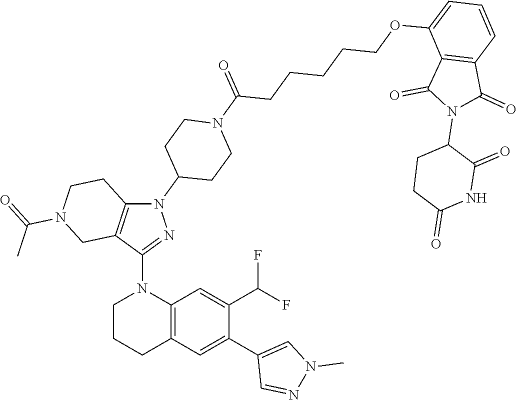 [SMILES: CC(=O)N1CCc2c(c(N3CCCc4cc(-c5cnn(C)c5)c(C(F)F)cc43)nn2C2CCN(C(=O)CCCCCOc3cccc4c3C(=O)N(C3CCC(=O)NC3=O)C4=O)CC2)C1] | 4-((6-(4-(5-acetyl-3-(7- (difluoromethyl)-6-(1-methyl-1H- pyrazol-4-yl)-3,4-dihydroquinolin- 1(2H)-yl)-4,5,6,7-tetrahydro-1H- pyrazolo[4,3-c]pyridin-1- yl)piperidin-1-yl)-6-oxohexyl)oxy)- 2-(2,6-dioxopiperidin-3- yl)isoindoline-1,3-dione |
| P-118 | 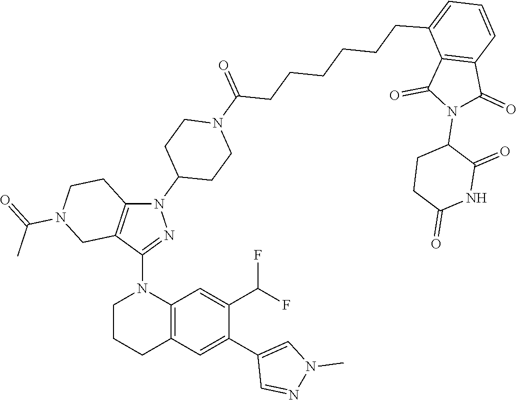 [SMILES: CC(=O)N1CCc2c(c(N3CCCc4cc(-c5cnn(C)c5)c(C(F)F)cc43)nn2C2CCN(C(=O)CCCCCCc3cccc4c3C(=O)N(C3CCC(=O)NC3=O)C4=O)CC2)C1] | 4-(7-(4-(5-acetyl-3-(7- (difluoromethyl)-6-(1-methyl-1H- pyrazol-4-yl)-3,4-dihydroquinolin- 1(2H)-yl)-4,5,6,7-tetrahydro-1H- pyrazolo[4,3-c]pyridin-1- yl)piperidin-1-yl)-7-oxoheptyl)-2- (2,6-dioxopiperidin-3- yl)isoindoline-1,3-dione |
| P-119 | 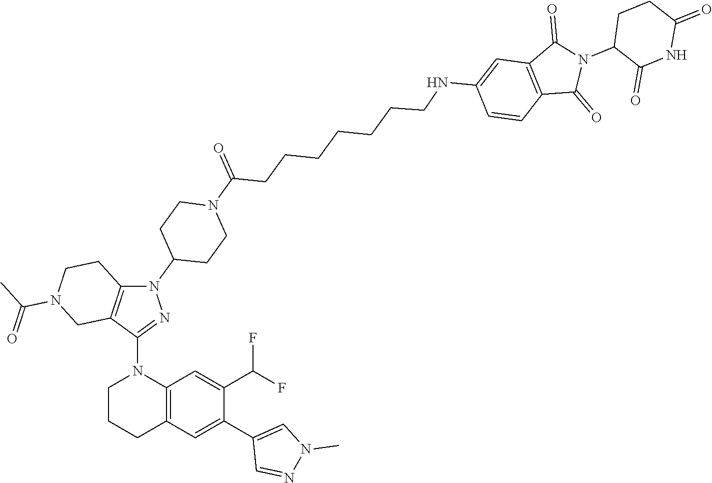 [SMILES: CC(=O)N1CCc2c(c(N3CCCc4cc(-c5cnn(C)c5)c(C(F)F)cc43)nn2C2CCN(C(=O)CCCCCCCNc3ccc4c(c3)C(=O)N(C3CCC(=O)NC3=O)C4=O)CC2)C1] | 5-((8-(4-(5-acetyl-3-(7- (difluoromethyl)-6-(1-methyl-1H- pyrazol-4-yl)-3,4-dihydroquinolin- 1(2H)-yl)-4,5,6,7-tetrahydro-1H- pyrazolo[4,3-c]pyridin-1- yl)piperidin-1-yl)-8- oxooctyl)amino)-2-(2,6- dioxopiperidin-3-yl)isoindoline-1,3- dione |
| P-120 | 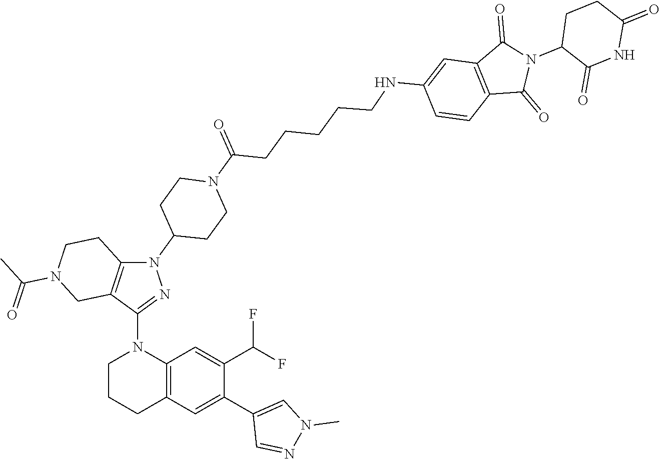 [SMILES: CC(=O)N1CCc2c(c(N3CCCc4cc(-c5cnn(C)c5)c(C(F)F)cc43)nn2C2CCN(C(=O)CCCCCNc3ccc4c(c3)C(=O)N(C3CCC(=O)NC3=O)C4=O)CC2)C1] | 5-((6-(4-(5-acetyl-3-(7- (difluoromethyl)-6-(1-methyl-1H- pyrazol-4-yl)-3,4-dihydroquinolin- 1(2H)-yl)-4,5,6,7-tetrahydro-1H- pyrazolo[4,3-c]pyridin-1- yl)piperidin-1-yl)-6- oxohexyl)amino)-2-(2,6- dioxopiperidin-3-yl)isoindoline-1,3- dione |
| P-121 | 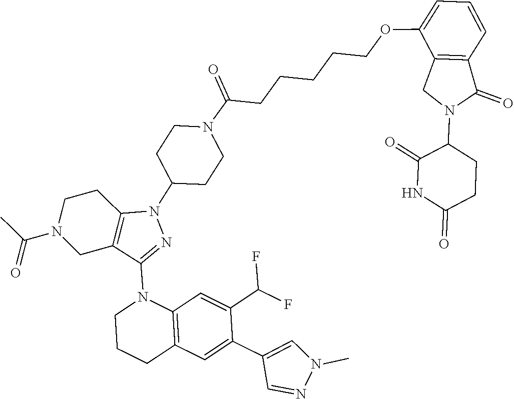 [SMILES: CC(=O)N1CCc2c(c(N3CCCc4cc(-c5cnn(C)c5)c(C(F)F)cc43)nn2C2CCN(C(=O)CCCCCOc3cccc4c3CN(C3CCC(=O)NC3=O)C4=O)CC2)C1] | 3-(4-((6-(4-(5-acetyl-3-(7- (difluoromethyl)-6-(1-methyl-1H- pyrazol-4-yl)-3,4-dihydroquinolin- 1(2H)-yl)-4,5,6,7-tetrahydro-1H- pyrazolo[4,3-c]pyridin-1- yl)piperidin-1-yl)-6-oxohexyl)oxy)- 1-oxoisoindolin-2-yl)piperidine-2,6- dione |
| P-122 | 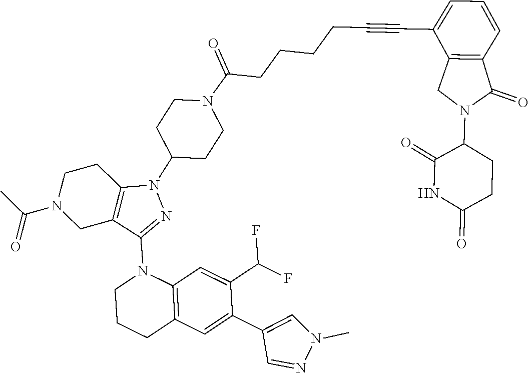 [SMILES: CC(=O)N1CCc2c(c(N3CCCc4cc(-c5cnn(C)c5)c(C(F)F)cc43)nn2C2CCN(C(=O)CCCCC#Cc3cccc4c3CN(C3CCC(=O)NC3=O)C4=O)CC2)C1] | 3-(4-(7-(4-(5-acetyl-3-(7- (difluoromethyl)-6-(1-methyl-1H- pyrazol-4-yl)-3,4-dihydroquinolin- 1(2H)-yl)-4,5,6,7-tetrahydro-1H- pyrazolo[4,3-c]pyridin-1- yl)piperidin-1-yl)-7-oxohept-1-yn-1- yl)-1-oxoisoindolin-2-yl)piperidine- 2,6-dione |
| P-123 | 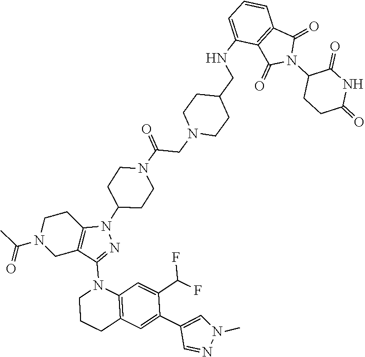 [SMILES: CC(=O)N1CCc2c(c(N3CCCc4cc(-c5cnn(C)c5)c(C(F)F)cc43)nn2C2CCN(C(=O)CN3CCC(CNc4cccc5c4C(=O)N(C4CCC(=O)NC4=O)C5=O)CC3)CC2)C1] | 4-(((1-(2-(4-(5-acetyl-3-(7- (difluoromethyl)-6-(1-methyl-1H- pyrazol-4-yl)-3,4-dihydroquinolin- 1(2H)-yl)-4,5,6,7-tetrahydro-1H- pyrazolo[4,3-c]pyridin-1- yl)piperidin-1-yl)-2- oxoethyl)piperidin-4- yl)methyl)amino)-2-(2,6- dioxopiperidin-3-yl)isoindoline-1,3- dione |
| P-124 | 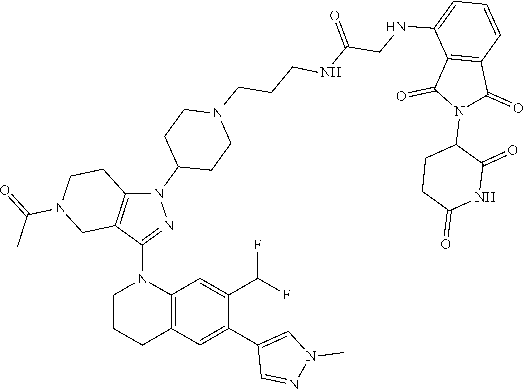 [SMILES: CC(=O)N1CCc2c(c(N3CCCc4cc(-c5cnn(C)c5)c(C(F)F)cc43)nn2C2CCN(CCCNC(=O)CNc3cccc4c3C(=O)N(C3CCC(=O)NC3=O)C4=O)CC2)C1] | N-(3-(4-(5-acetyl-3-(7- (difluoromethyl)-6-(1-methyl-1H- pyrazol-4-yl)-3,4-dihydroquinolin- 1(2H)-yl)-4,5,6,7-tetrahydro-1H- pyrazolo[4,3-c]pyridin-1- yl)piperidin-1-yl)propyl)-2-((2-(2,6- dioxopiperidin-3-yl)-1,3- dioxoisoindolin-4- yl)amino)acetamide |
| P-125 | 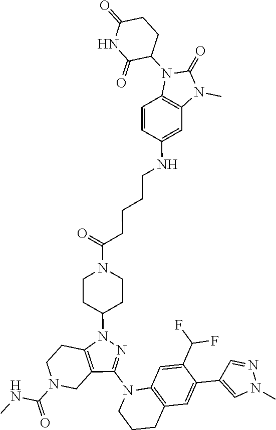 | 3-(7-(difluoromethyl)-6-(1-methyl- 1H-pyrazol-4-yl)-3,4- dihydroquinolin-1(2H)-yl)-1-(1-(5- ((1-(2,6-dioxopiperidin-3-yl)-3- methyl-2-oxo-2,3-dihydro-1H- benzo[d]imidazol-5- yl)amino)pentanoyl)piperidin-4-yl)- N-methyl-1,4,6,7-tetrahydro-5H- pyrazolo[4,3-c]pyridine-5- carboxamide |
| P-126 | 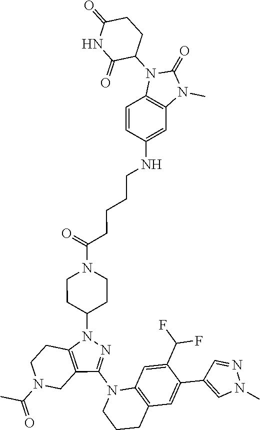 [SMILES: CC(=O)N1CCc2c(c(N3CCCc4cc(-c5cnn(C)c5)c(C(F)F)cc43)nn2C2CCN(C(=O)CCCCNc3ccc4c(c3)n(C)c(=O)n4C3CCC(=O)NC3=O)CC2)C1] | 3-(5-((5-(4-(5-acetyl-3-(7- (difluoromethyl)-6-(1-methyl-1H- pyrazol-4-yl)-3,4-dihydroquinolin- 1(2H)-yl)-4,5,6,7-tetrahydro-1H- pyrazolo[4,3-c]pyridin-1- yl)piperidin-1-yl)-5- oxopentyl)amino)-3-methyl-2-oxo- 2,3-dihydro-1H-benzo[d]imidazol- 1-yl)piperidine-2,6-dione |
| P-127 | 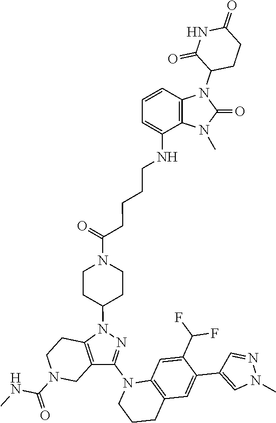 | 3-(7-(difluoromethyl)-6-(1-methyl- 1H-pyrazol-4-yl)-3,4- dihydroquinolin-1(2H)-yl)-1-(1-(5- ((1-(2,6-dioxopiperidin-3-yl)-3- methyl-2-oxo-2,3-dihydro-1H- benzo[d]imidazol-4- yl)amino)pentanoyl)piperidin-4-yl)- N-methyl-1,4,6,7-tetrahydro-5H- pyrazolo[4,3-c]pyridine-5- carboxamide |
| P-128 | 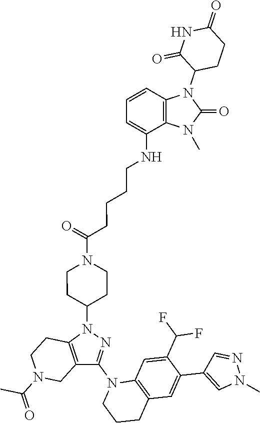 [SMILES: CC(=O)N1CCc2c(c(N3CCCc4cc(-c5cnn(C)c5)c(C(F)F)cc43)nn2C2CCN(C(=O)CCCCNc3cccc4c3n(C)c(=O)n4C3CCC(=O)NC3=O)CC2)C1] | 3-(4-((5-(4-(5-acetyl-3-(7- (difluoromethyl)-6-(1-methyl-1H- pyrazol-4-yl)-3,4-dihydroquinolin- 1(2H)-yl)-4,5,6,7-tetrahydro-1H- pyrazolo[4,3-c]pyridin-1- yl)piperidin-1-yl)-5- oxopentyl)amino)-3-methyl-2-oxo- 2,3-dihydro-1H-benzo[d]imidazol- 1-yl)piperidine-2,6-dione |
| P-129 | 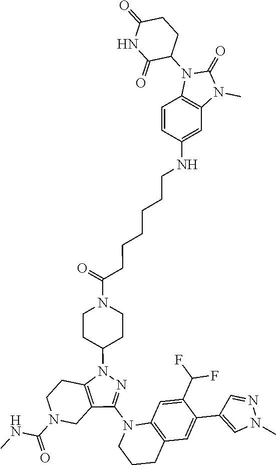 | 3-(7-(difluoromethyl)-6-(1-methyl- 1H-pyrazol-4-yl)-3,4- dihydroquinolin-1(2H)-yl)-1-(1-(7- ((1-(2,6-dioxopiperidin-3-yl)-3- methyl-2-oxo-2,3-dihydro-1H- benzo[d]imidazol-5- yl)amino)heptanoyl)piperidin-4-yl)- N-methyl-1,4,6,7-tetrahydro-5H- pyrazolo[4,3-c]pyridine-5- carboxamide |
| P-130 | 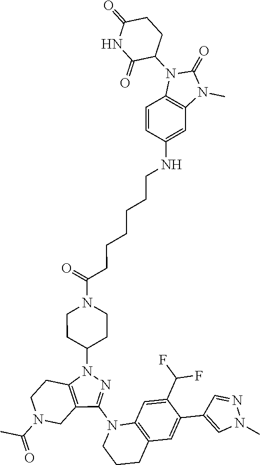 [SMILES: CC(=O)N1CCc2c(c(N3CCCc4cc(-c5cnn(C)c5)c(C(F)F)cc43)nn2C2CCN(C(=O)CCCCCCNc3ccc4c(c3)n(C)c(=O)n4C3CCC(=O)NC3=O)CC2)C1] | 3-(5-((7-(4-(5-acetyl-3-(7- (difluoromethyl)-6-(1-methyl-1H- pyrazol-4-yl)-3,4-dihydroquinolin- 1(2H)-yl)-4,5,6,7-tetrahydro-1H- pyrazolo[4,3-c]pyridin-1- yl)piperidin-1-yl)-7- oxoheptyl)amino)-3-methyl-2-oxo- 2,3-dihydro-1H-benzo[d]imidazol- 1-yl)piperidine-2,6-dione |
| P-131 | 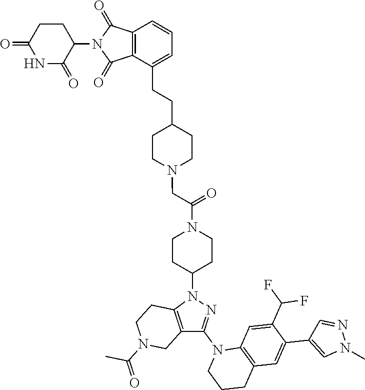 [SMILES: CC(=O)N1CCc2c(c(N3CCCc4cc(-c5cnn(C)c5)c(C(F)F)cc43)nn2C2CCN(C(=O)CN3CCC(CCc4cccc5c4C(=O)N(C4CCC(=O)NC4=O)C5=O)CC3)CC2)C1] | 4-(2-(1-(2-(4-(5-acetyl-3-(7- (difluoromethyl)-6-(1-methyl-1H- pyrazol-4-yl)-3,4-dihydroquinolin- 1(2H)-yl)-4,5,6,7-tetrahydro-1H- pyrazolo[4,3-c]pyridin-1- yl)piperidin-1-yl)-2- oxoethyl)piperidin-4-yl)ethyl)-2- (2,6-dioxopiperidin-3- yl)isoindoline-1,3-dione |
| P-132 | 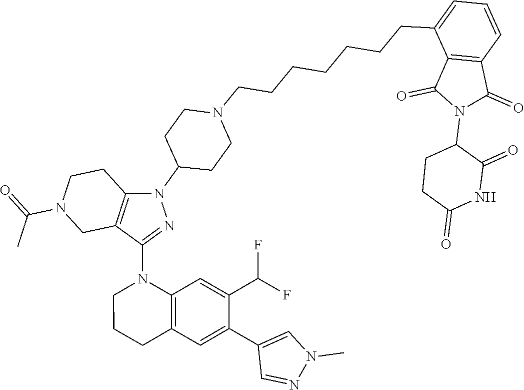 [SMILES: CC(=O)N1CCc2c(c(N3CCCc4cc(-c5cnn(C)c5)c(C(F)F)cc43)nn2C2CCN(CCCCCCCc3cccc4c3C(=O)N(C3CCC(=O)NC3=O)C4=O)CC2)C1] | 4-(7-(4-(5-acetyl-3-(7- (difluoromethyl)-6-(1-methyl-1H- pyrazol-4-yl)-3,4-dihydroquinolin- 1(2H)-yl)-4,5,6,7-tetrahydro-1H- pyrazolo[4,3-c]pyridin-1- yl)piperidin-1-yl)heptyl)-2-(2,6- dioxopiperidin-3-yl)isoindoline-1,3- dione |
| P-133 | 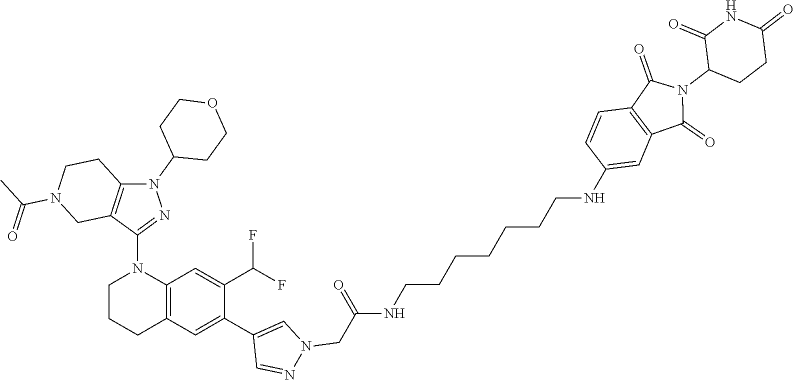 [SMILES: CC(=O)N1CCc2c(c(N3CCCc4cc(-c5cnn(CC(=O)NCCCCCCCNc6ccc7c(c6)C(=O)N(C6CCC(=O)NC6=O)C7=O)c5)c(C(F)F)cc43)nn2C2CCOCC2)C1] | 2-(4-(1-(5-acetyl-1-(tetrahydro-2H- pyran-4-yl)-4,5,6,7-tetrahydro-1H- pyrazolo[4,3-c]pyridin-3-yl)-7- (difluoromethyl)-1,2,3,4- tetrahydroquinolin-6-yl)-1H- pyrazol-1-yl)-N-(7-((2-(2,6- dioxopiperidin-3-yl)-1,3- dioxoisoindolin-5- yl)amino)heptyl)acetamide |
| P-134 | 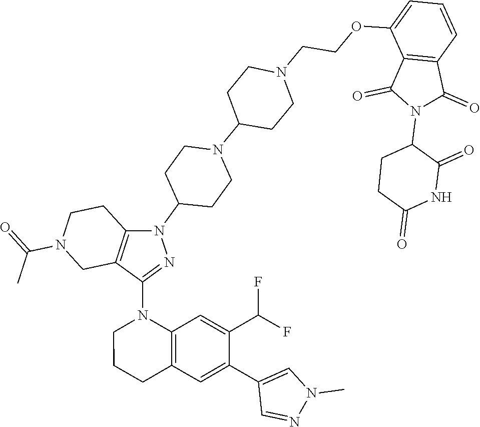 [SMILES: CC(=O)N1CCc2c(c(N3CCCc4cc(-c5cnn(C)c5)c(C(F)F)cc43)nn2C2CCN(C3CCN(CCOc4cccc5c4C(=O)N(C4CCC(=O)NC4=O)C5=O)CC3)CC2)C1] | 4-(2-(4-(5-acetyl-3-(7- (difluoromethyl)-6-(1-methyl-1H- pyrazol-4-yl)-3,4-dihydroquinolin- 1(2H)-yl)-4,5,6,7-tetrahydro-1H- pyrazolo[4,3-c]pyridin-1-yl)-[1,4′- bipiperidin]-1′-yl)ethoxy)-2-(2,6- dioxopiperidin-3-yl)isoindoline-1,3- dione |
| P-135 | 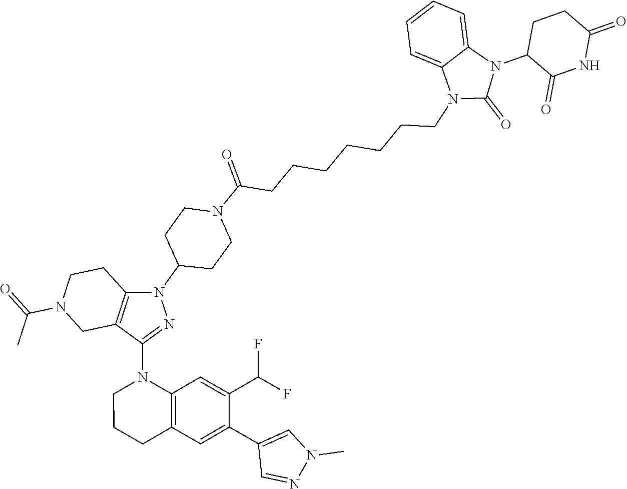 [SMILES: CC(=O)N1CCc2c(c(N3CCCc4cc(-c5cnn(C)c5)c(C(F)F)cc43)nn2C2CCN(C(=O)CCCCCCCn3c(=O)n(C4CCC(=O)NC4=O)c4ccccc43)CC2)C1] | 3-(3-(8-(4-(5-acetyl-3-(7- (difluoromethyl)-6-(1-methyl-1H- pyrazol-4-yl)-3,4-dihydroquinolin- 1(2H)-yl)-4,5,6,7-tetrahydro-1H- pyrazolo[4,3-c]pyridin-1- yl)piperidin-1-yl)-8-oxooctyl)-2- oxo-2,3-dihydro-1H- benzo[d]imidazol-1-yl)piperidine- 2,6-dione |
| P-136 | 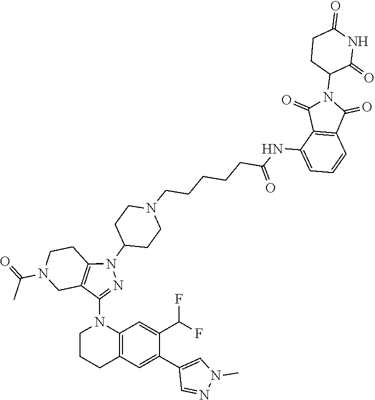 [SMILES: CC(=O)N1CCc2c(c(N3CCCc4cc(-c5cnn(C)c5)c(C(F)F)cc43)nn2C2CCN(CCCCCC(=O)Nc3cccc4c3C(=O)N(C3CCC(=O)NC3=O)C4=O)CC2)C1] | 6-(4-(5-acetyl-3-(7- (difluoromethyl)-6-(1-methyl-1H- pyrazol-4-yl)-3,4-dihydroquinolin- 1(2H)-yl)-4,5,6,7-tetrahydro-1H- pyrazolo[4,3-c]pyridin-1- yl)piperidin-1-yl)-N-(2-(2,6- dioxopiperidin-3-yl)-1,3- dioxoisoindolin-4-yl)hexanamide |
| P-137 | 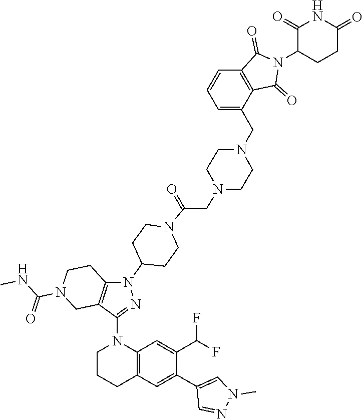 [SMILES: CNC(=O)N1CCc2c(c(N3CCCc4cc(-c5cnn(C)c5)c(C(F)F)cc43)nn2C2CCN(C(=O)CN3CCN(Cc4cccc5c4C(=O)N(C4CCC(=O)NC4=O)C5=O)CC3)CC2)C1] | 3-(7-(difluoromethyl)-6-(1-methyl- 1H-pyrazol-4-yl)-3,4- dihydroquinolin-1(2H)-yl)-1-(1-(2- (4-((2-(2,6-dioxopiperidin-3-yl)-1,3- dioxoisoindolin-4- yl)methyl)piperazin-1- yl)acetyl)piperidin-4-yl)-N-methyl- 1,4,6,7-tetrahydro-5H-pyrazolo[4,3- c]pyridine-5-carboxamide |
| P-138 | 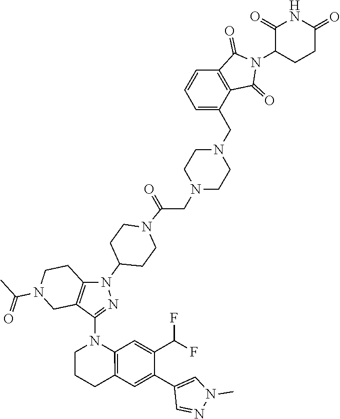 [SMILES: CC(=O)N1CCc2c(c(N3CCCc4cc(-c5cnn(C)c5)c(C(F)F)cc43)nn2C2CCN(C(=O)CN3CCN(Cc4cccc5c4C(=O)N(C4CCC(=O)NC4=O)C5=O)CC3)CC2)C1] | 4-((4-(2-(4-(5-acetyl-3-(7- (difluoromethyl)-6-(1-methyl-1H- pyrazol-4-yl)-3,4-dihydroquinolin- 1(2H)-yl)-4,5,6,7-tetrahydro-1H- pyrazolo[4,3-c]pyridin-1- yl)piperidin-1-yl)-2- oxoethyl)piperazin-1-yl)methyl)-2- (2,6-dioxopiperidin-3- yl)isoindoline-1,3-dione |
| P-139 | 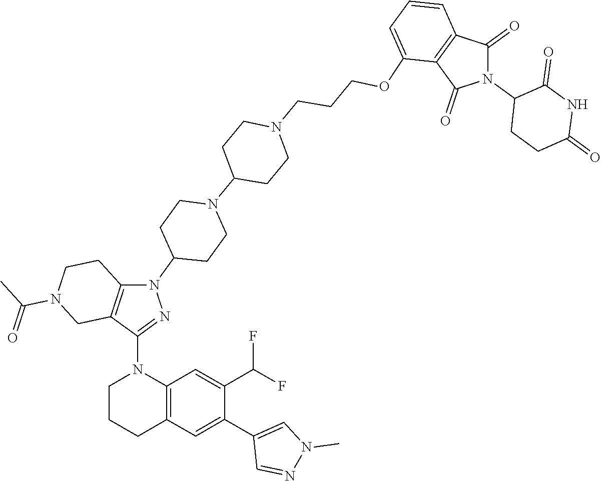 [SMILES: CC(=O)N1CCc2c(c(N3CCCc4cc(-c5cnn(C)c5)c(C(F)F)cc43)nn2C2CCN(C3CCN(CCCOc4cccc5c4C(=O)N(C4CCC(=O)NC4=O)C5=O)CC3)CC2)C1] | 4-(3-(4-(5-acetyl-3-(7- (difluoromethyl)-6-(1-methyl-1H- pyrazol-4-yl)-3,4-dihydroquinolin- 1(2H)-yl)-4,5,6,7-tetrahydro-1H- pyrazolo[4,3-c]pyridin-1-yl)-[1,4′- bipiperidin]-1′-yl)propoxy)-2-(2,6- dioxopiperidin-3-yl)isoindoline-1,3- dione |
| P-140 | 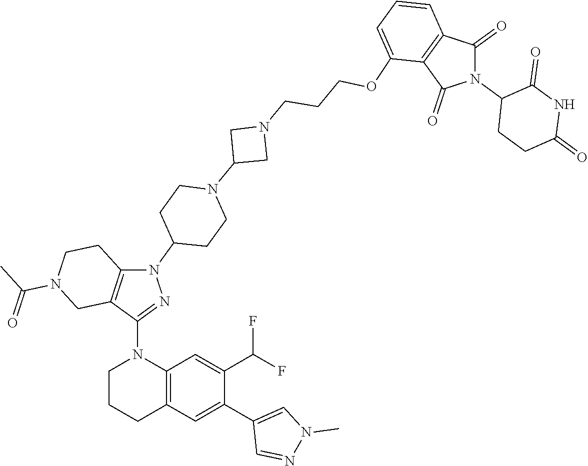 [SMILES: CC(=O)N1CCc2c(c(N3CCCc4cc(-c5cnn(C)c5)c(C(F)F)cc43)nn2C2CCN(C3CN(CCCOc4cccc5c4C(=O)N(C4CCC(=O)NC4=O)C5=O)C3)CC2)C1] | 4-(3-(3-(4-(5-acetyl-3-(7- (difluoromethyl)-6-(1-methyl-1H- pyrazol-4-yl)-3,4-dihydroquinolin- 1(2H)-yl)-4,5,6,7-tetrahydro-1H- pyrazolo[4,3-c]pyridin-1 yl)piperidin-1-yl)azetidin-1- yl)propoxy)-2-(2,6-dioxopiperidin- 3-yl)isoindoline-1,3-dione |
| P-141 | 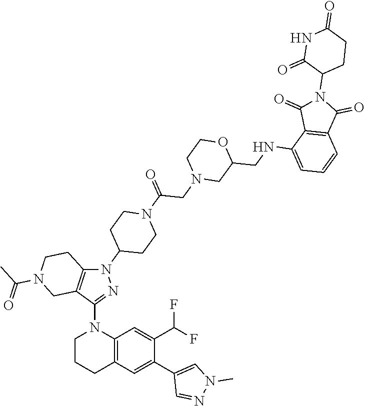 [SMILES: CC(=O)N1CCc2c(c(N3CCCc4cc(-c5cnn(C)c5)c(C(F)F)cc43)nn2C2CCN(C(=O)CN3CCOC(CNc4cccc5c4C(=O)N(C4CCC(=O)NC4=O)C5=O)C3)CC2)C1] | 4-(((4-(2-(4-(5-acetyl-3-(7- (difluoromethyl)-6-(1-methyl-1H- pyrazol-4-yl)-3,4-dihydroquinolin- 1(2H)-yl)-4,5,6,7-tetrahydro-1H- pyrazolo[4,3-c]pyridin-1- yl)piperidin-1-yl)-2- oxoethyl)morpholin-2- yl)methyl)amino)-2-(2,6- dioxopiperidin-3-yl)isoindoline-1,3- dione |
| P-142 | 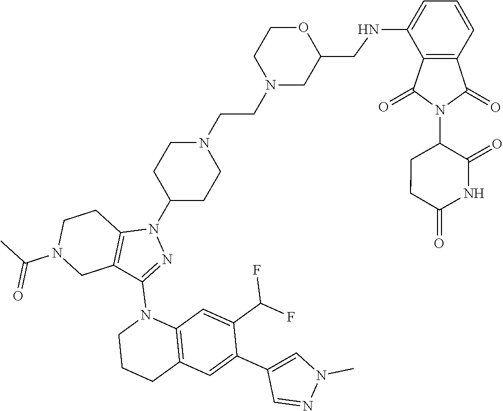 [SMILES: CC(=O)N1CCc2c(c(N3CCCc4cc(-c5cnn(C)c5)c(C(F)F)cc43)nn2C2CCN(CCN3CCOC(CNc4cccc5c4C(=O)N(C4CCC(=O)NC4=O)C5=O)C3)CC2)C1] | 4-(((4-(2-(4-(5-acetyl-3-(7- (difluoromethyl)-6-(1-methyl-1H- pyrazol-4-yl)-3,4-dihydroquinolin- 1(2H)-yl)-4,5,6,7-tetrahydro-1H- pyrazolo[4,3-c]pyridin-1- yl)piperidin-1-yl)-2- oxoethyl)morpholin-2- yl)methyl)amino)-2-(2,6- dioxopiperidin-3-yl)isoindoline-1,3- dione |
| P-143 | 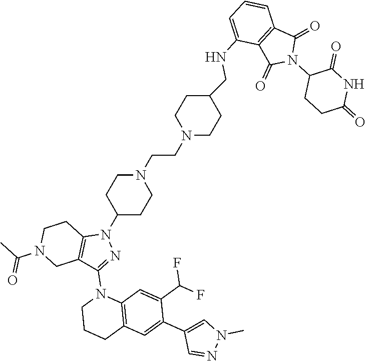 [SMILES: CC(=O)N1CCc2c(c(N3CCCc4cc(-c5cnn(C)c5)c(C(F)F)cc43)nn2C2CCN(CCN3CCC(CNc4cccc5c4C(=O)N(C4CCC(=O)NC4=O)C5=O)CC3)CC2)C1] | 4-(((1-(2-(4-(5-acetyl-3-(7- (difluoromethyl)-6-(1-methyl-1H- pyrazol-4-yl)-3,4-dihydroquinolin- 1(2H)-yl)-4,5,6,7-tetrahydro-1H- pyrazolo[4,3-c]pyridin-1- yl)piperidin-1-yl)ethyl)piperidin-4- yl)methyl)amino)-2-(2,6- dioxopiperidin-3-yl)isoindoline-1,3- dione |
| P-144 | 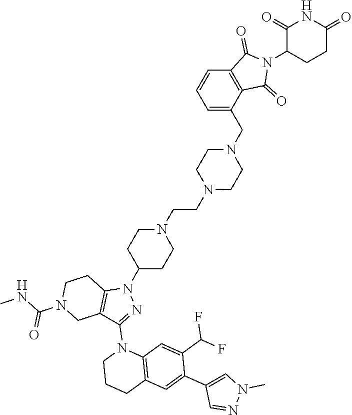 [SMILES: CNC(=O)N1CCc2c(c(N3CCCc4cc(-c5cnn(C)c5)c(C(F)F)cc43)nn2C2CCN(CCN3CCN(Cc4cccc5c4C(=O)N(C4CCC(=O)NC4=O)C5=O)CC3)CC2)C1] | 3-(7-(difluoromethyl)-6-(1-methyl- 1H-pyrazol-4-yl)-3,4- dihydroquinolin-1(2H)-yl)-1-(1-(2- (4-((2-(2,6-dioxopiperidin-3-yl)-1,3- dioxoisoindolin-4- yl)methyl)piperazin-1- yl)ethyl)piperidin-4-yl)-N-methyl- 1,4,6,7-tetrahydro-5H-pyrazolo[4,3- c]pyridine-5-carboxamide |
| P-145 | 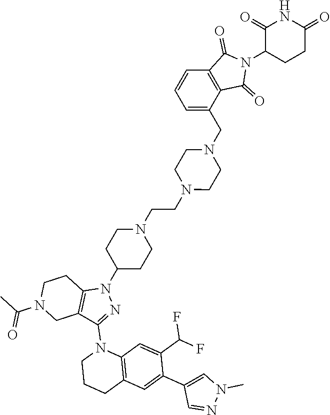 [SMILES: CC(=O)N1CCc2c(c(N3CCCc4cc(-c5cnn(C)c5)c(C(F)F)cc43)nn2C2CCN(CCN3CCN(Cc4cccc5c4C(=O)N(C4CCC(=O)NC4=O)C5=O)CC3)CC2)C1] | 4-((4-(2-(4-(5-acetyl-3-(7- (difluoromethyl)-6-(1-methyl-1H- pyrazol-4-yl)-3,4-dihydroquinolin- 1(2H)-yl)-4,5,6,7-tetrahydro-1H- pyrazolo[4,3-c]pyridin-1- yl)piperidin-1-yl)ethyl)piperazin-1- yl)methyl)-2-(2,6-dioxopiperidin-3- yl)isoindoline-1,3-dione |
| P-146 | 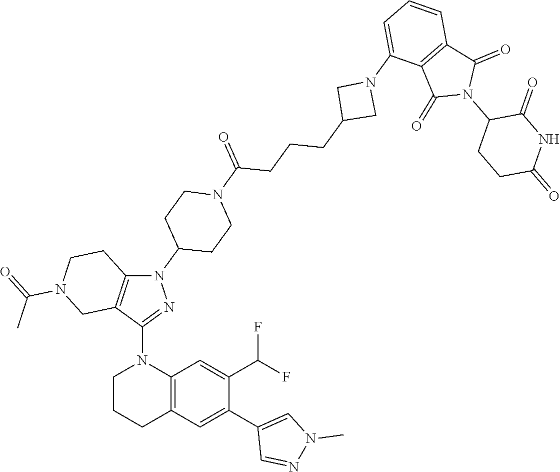 [SMILES: CC(=O)N1CCc2c(c(N3CCCc4cc(-c5cnn(C)c5)c(C(F)F)cc43)nn2C2CCN(C(=O)CCCC3CN(c4cccc5c4C(=O)N(C4CCC(=O)NC4=O)C5=O)C3)CC2)C1] | 4-(3-(4-(4-(5-acetyl-3-(7- (difluoromethyl)-6-(1-methyl-1H- pyrazol-4-yl)-3,4-dihydroquinolin- 1(2H)-yl)-4,5,6,7-tetrahydro-1H- pyrazolo[4,3-c]pyridin-1- yl)piperidin-1-yl)-4- oxobutyl)azetidin-1-yl)-2-(2,6- dioxopiperidin-3-yl)isoindoline-1,3- dione |
| P-147 | 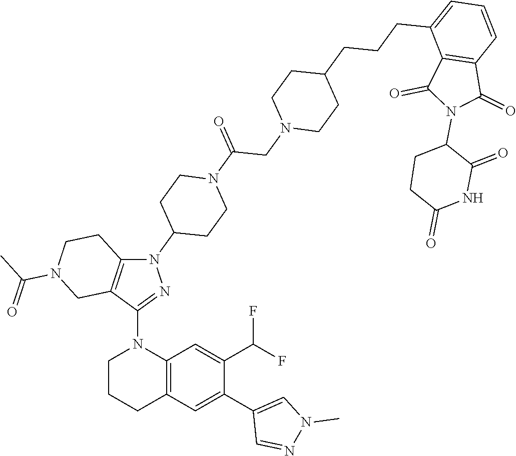 [SMILES: CC(=O)N1CCc2c(c(N3CCCc4cc(-c5cnn(C)c5)c(C(F)F)cc43)nn2C2CCN(C(=O)CN3CCC(CCCc4cccc5c4C(=O)N(C4CCC(=O)NC4=O)C5=O)CC3)CC2)C1] | 4-(3-(1-(2-(4-(5-acetyl-3-(7- (difluoromethyl)-6-(1-methyl-1H- pyrazol-4-yl)-3,4-dihydroquinolin- 1(2H)-yl)-4,5,6,7-tetrahydro-1H- pyrazolo[4,3-c]pyridin-1- yl)piperidin-1-yl)-2- oxoethyl)piperidin-4-yl)propyl)-2- (2,6-dioxopiperidin-3- yl)isoindoline-1,3-dione |
| P-148 | 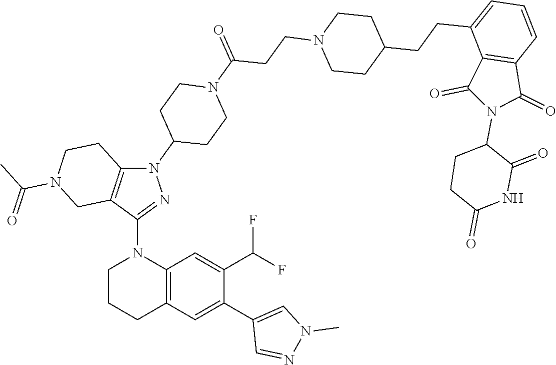 [SMILES: CC(=O)N1CCc2c(c(N3CCCc4cc(-c5cnn(C)c5)c(C(F)F)cc43)nn2C2CCN(C(=O)CCN3CCC(CCc4cccc5c4C(=O)N(C4CCC(=O)NC4=O)C5=O)CC3)CC2)C1] | 4-(2-(1-(3-(4-(5-acetyl-3-(7- (difluoromethyl)-6-(1-methyl-1H- pyrazol-4-yl)-3,4-dihydroquinolin- 1(2H)-yl)-4,5,6,7-tetrahydro-1H- pyrazolo[4,3-c]pyridin-1- yl)piperidin-1-yl)-3- oxopropyl)piperidin-4-yl)ethyl)-2- (2,6-dioxopiperidin-3- yl)isoindoline-1,3-dione |
| P-149 | 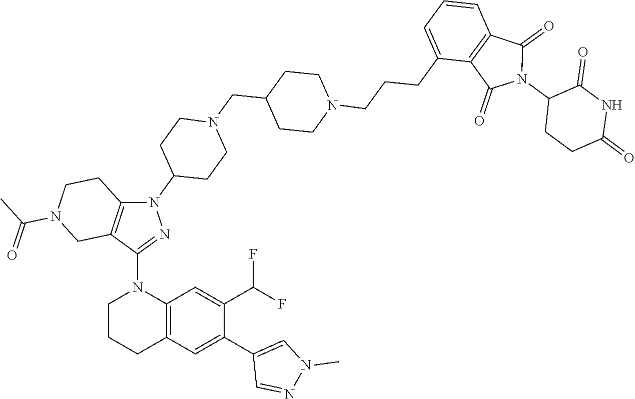 [SMILES: CC(=O)N1CCc2c(c(N3CCCc4cc(-c5cnn(C)c5)c(C(F)F)cc43)nn2C2CCN(CC3CCN(CCCc4cccc5c4C(=O)N(C4CCC(=O)NC4=O)C5=O)CC3)CC2)C1] | 4-(3-(4-((4-(5-acetyl-3-(7- (difluoromethyl)-6-(1-methyl-1H- pyrazol-4-yl)-3,4-dihydroquinolin- 1(2H)-yl)-4,5,6,7-tetrahydro-1H- pyrazolo[4,3-c]pyridin-1- yl)piperidin-1-yl)methyl)piperidin- 1-yl)propyl)-2-(2,6-dioxopiperidin- 3-yl)isoindoline-1,3-dione |
| P-150 | 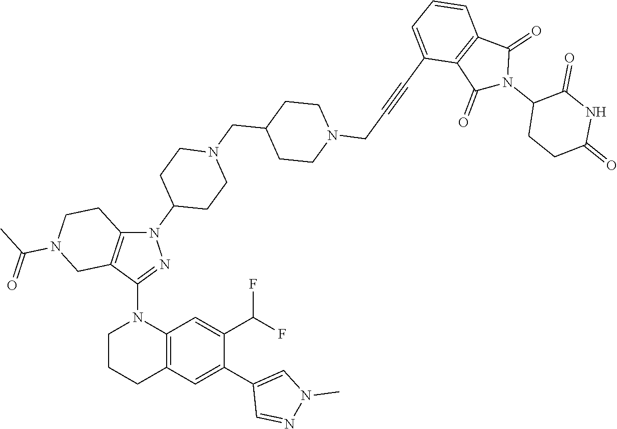 [SMILES: CC(=O)N1CCc2c(c(N3CCCc4cc(-c5cnn(C)c5)c(C(F)F)cc43)nn2C2CCN(CC3CCN(CC#Cc4cccc5c4C(=O)N(C4CCC(=O)NC4=O)C5=O)CC3)CC2)C1] | 4-(3-(4-((4-(5-acetyl-3-(7- (difluoromethyl)-6-(1-methyl-1H- pyrazol-4-yl)-3,4-dihydroquinolin- 1(2H)-yl)-4,5,6,7-tetrahydro-1H- pyrazolo[4,3-c]pyridin-1- yl)piperidin-1-yl)methyl)piperidin- 1-yl)prop-1-yn-1-yl)-2-(2,6- dioxopiperidin-3-yl)isoindoline-1,3- dione |
| P-151 | 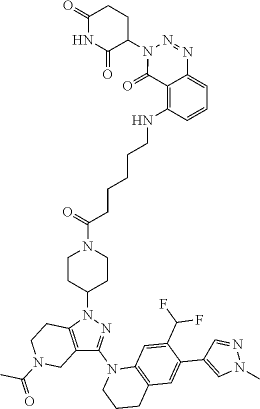 [SMILES: CC(=O)N1CCc2c(c(N3CCCc4cc(-c5cnn(C)c5)c(C(F)F)cc43)nn2C2CCN(C(=O)CCCCCNc3cccc4nnn(C5CCC(=O)NC5=O)c(=O)c34)CC2)C1] | 3-(5-((6-(4-(5-acetyl-3-(7- (difluoromethyl)-6-(1-methyl-1H- pyrazol-4-yl)-3,4-dihydroquinolin- 1(2H)-yl)-4,5,6,7-tetrahydro-1H- pyrazolo[4,3-c]pyridin-1- yl)piperidin-1-yl)-6- oxohexyl)amino)-4- oxobenzo[d][1,2,3]triazin-3(4H)- yl)piperidine-2,6-dione |
| P-152 | 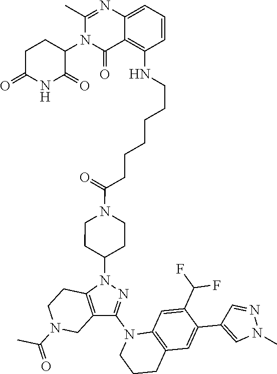 [SMILES: CC(=O)N1CCc2c(c(N3CCCc4cc(-c5cnn(C)c5)c(C(F)F)cc43)nn2C2CCN(C(=O)CCCCCCNc3cccc4nc(C)n(C5CCC(=O)NC5=O)c(=O)c34)CC2)C1] | 3-(5-((7-(4-(5-acetyl-3-(7- (difluoromethyl)-6-(1-methyl-1H- pyrazol-4-yl)-3,4-dihydroquinolin- 1(2H)-yl)-4,5,6,7-tetrahydro-1H- pyrazolo[4,3-c]pyridin-1- yl)piperidin-1-yl)-7- oxoheptyl)amino)-2-methyl-4- oxoquinazolin-3(4H)-yl)piperidine- 2,6-dione |
| P-153 | 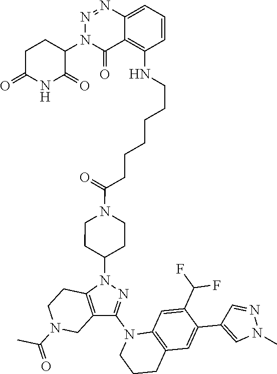 [SMILES: CC(=O)N1CCc2c(c(N3CCCc4cc(-c5cnn(C)c5)c(C(F)F)cc43)nn2C2CCN(C(=O)CCCCCCNc3cccc4nnn(C5CCC(=O)NC5=O)c(=O)c34)CC2)C1] | 3-(5-((7-(4-(5-acetyl-3-(7- (difluoromethyl)-6-(1-methyl-1H- pyrazol-4-yl)-3,4-dihydroquinolin- 1(2H)-yl)-4,5,6,7-tetrahydro-1H- pyrazolo[4,3-c]pyridin-1- yl)piperidin-1-yl)-7- oxoheptyl)amino)-4- oxobenzo[d][1,2,3]triazin-3(4H)- yl)piperidine-2,6-dione |
| P-154 | 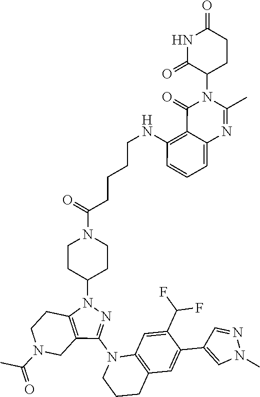 [SMILES: CC(=O)N1CCc2c(c(N3CCCc4cc(-c5cnn(C)c5)c(C(F)F)cc43)nn2C2CCN(C(=O)CCCCNc3cccc4nc(C)n(C5CCC(=O)NC5=O)c(=O)c34)CC2)C1] | 3-(5-((5-(4-(5-acetyl-3-(7- (difluoromethyl)-6-(1-methyl-1H- pyrazol-4-yl)-3,4-dihydroquinolin- 1(2H)-yl)-4,5,6,7-tetrahydro-1H- pyrazolo[4,3-c]pyridin-1- yl)piperidin-1-yl)-5- oxopentyl)amino)-2-methyl-4- oxoquinazolin-3(4H)-yl)piperidine- 2,6-dione |
| P-155 | 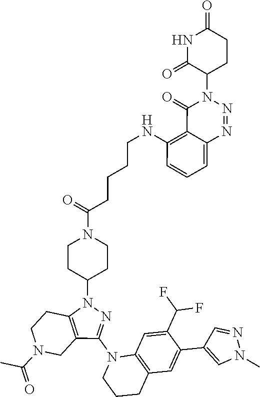 [SMILES: CC(=O)N1CCc2c(c(N3CCCc4cc(-c5cnn(C)c5)c(C(F)F)cc43)nn2C2CCN(C(=O)CCCCNc3cccc4nnn(C5CCC(=O)NC5=O)c(=O)c34)CC2)C1] | 3-(5-((5-(4-(5-acetyl-3-(7- (difluoromethyl)-6-(1-methyl-1H- pyrazol-4-yl)-3,4-dihydroquinolin- 1(2H)-yl)-4,5,6,7-tetrahydro-1H- pyrazolo[4,3-c]pyridin-1- yl)piperidin-1-yl)-5- oxopentyl)amino)-4- oxobenzo[d][1,2,3]triazin-3(4H)- yl)piperidine-2,6-dione |
| P-156 | 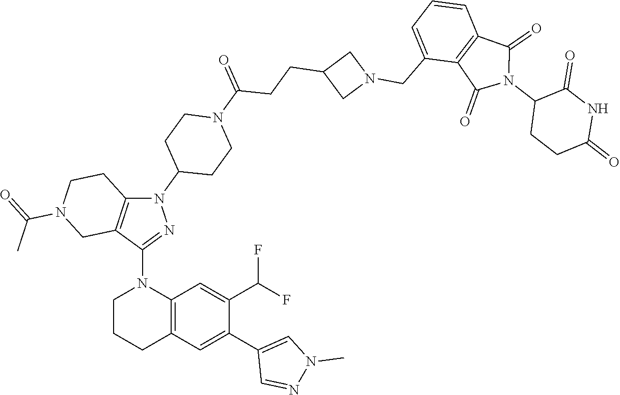 [SMILES: CC(=O)N1CCc2c(c(N3CCCc4cc(-c5cnn(C)c5)c(C(F)F)cc43)nn2C2CCN(C(=O)CCC3CN(Cc4cccc5c4C(=O)N(C4CCC(=O)NC4=O)C5=O)C3)CC2)C1] | 4-((3-(3-(4-(5-acetyl-3-(7- (difluoromethyl)-6-(1-methyl-1H- pyrazol-4-yl)-3,4-dihydroquinolin- 1(2H)-yl)-4,5,6,7-tetrahydro-1H- pyrazolo[4,3-c]pyridin-1- yl)piperidin-1-yl)-3- oxopropyl)azetidin-1-yl)methyl)-2- (2,6-dioxopiperidin-3- yl)isoindoline-1,3-dione |
| P-157 | 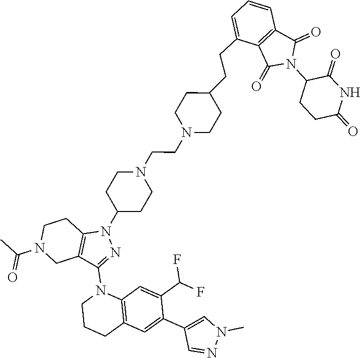 [SMILES: CC(=O)N1CCc2c(c(N3CCCc4cc(-c5cnn(C)c5)c(C(F)F)cc43)nn2C2CCN(CCN3CCC(CCc4cccc5c4C(=O)N(C4CCC(=O)NC4=O)C5=O)CC3)CC2)C1] | 4-(2-(1-(2-(4-(5-acetyl-3-(7- (difluoromethyl)-6-(1-methyl-1H- pyrazol-4-yl)-3,4-dihydroquinolin- 1(2H)-yl)-4,5,6,7-tetrahydro-1H- pyrazolo[4,3-c]pyridin-1- yl)piperidin-1-yl)ethyl)piperidin-4- yl)isoindoline-1,3-dione |
| P-158 | 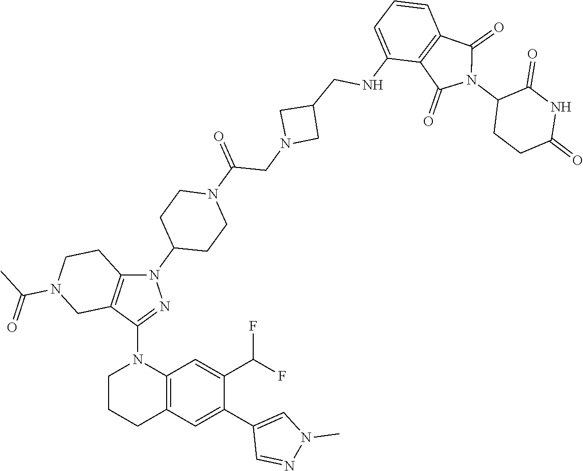 [SMILES: CC(=O)N1CCc2c(c(N3CCCc4cc(-c5cnn(C)c5)c(C(F)F)cc43)nn2C2CCN(C(=O)CN3CC(CNc4cccc5c4C(=O)N(C4CCC(=O)NC4=O)C5=O)C3)CC2)C1] | 4-(((1-(2-(4-(5-acetyl-3-(7- (difluoromethyl)-6-(1-methyl-1H- pyrazol-4-yl)-3,4-dihydroquinolin- 1(2H)-yl)-4,5,6,7-tetrahydro-1H- pyrazolo[4,3-c]pyridin-1- yl)piperidin-1-yl)-2- oxoethyl)azetidin-3- yl)methyl)amino)-2-(2,6- dioxopiperidin-3-yl)isoindoline-1,3- dione |
| P-159 | 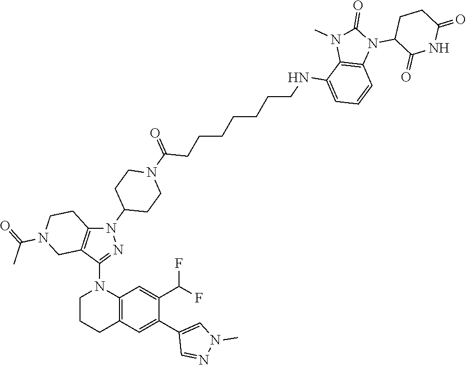 [SMILES: CC(=O)N1CCc2c(c(N3CCCc4cc(-c5cnn(C)c5)c(C(F)F)cc43)nn2C2CCN(C(=O)CCCCCCCNc3cccc4c3n(C)c(=O)n4C3CCC(=O)NC3=O)CC2)C1] | 3-(4-((8-(4-(5-acetyl-3-(7- (difluoromethyl)-6-(1-methyl-1H- pyrazol-4-yl)-3,4-dihydroquinolin- 1(2H)-yl)-4,5,6,7-tetrahydro-1H- pyrazolo[4,3-c]pyridin-1- yl)piperidin-1-yl)-8- oxooctyl)amino)-3-methyl-2-oxo- 2,3-dihydro-1H-benzo[d]imidazol- 1-yl)piperidine-2,6-dione |
| P-160 | 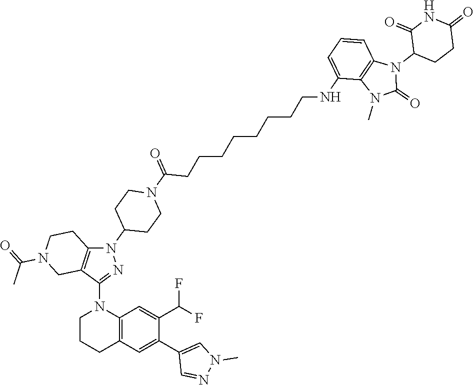 [SMILES: CC(=O)N1CCc2c(c(N3CCCc4cc(-c5cnn(C)c5)c(C(F)F)cc43)nn2C2CCN(C(=O)CCCCCCCCNc3cccc4c3n(C)c(=O)n4C3CCC(=O)NC3=O)CC2)C1] | 3-(4-((9-(4-(5-acetyl-3-(7- (difluoromethyl)-6-(1-methyl-1H- pyrazol-4-yl)-3,4-dihydroquinolin- 1(2H)-yl)-4,5,6,7-tetrahydro-1H- pyrazolo[4,3-c]pyridin-1- yl)piperidin-1-yl)-9- oxononyl)amino)-3-methyl-2-oxo- 2,3-dihydro-1H-benzo[d]imidazol- 1-yl)piperidine-2,6-dione |
| P-161 | 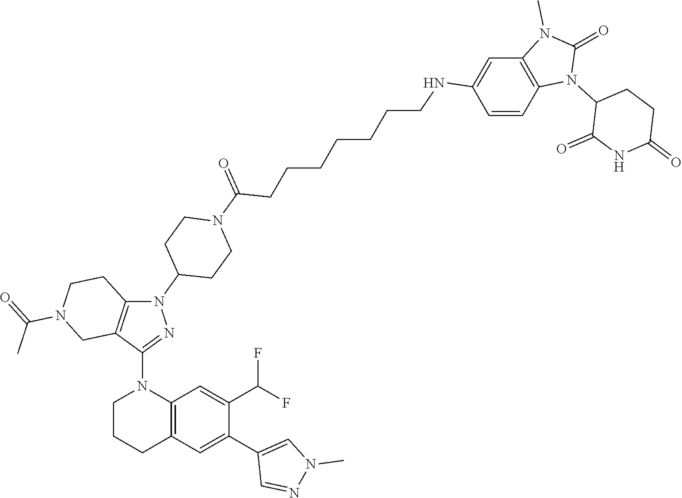 [SMILES: CC(=O)N1CCc2c(c(N3CCCc4cc(-c5cnn(C)c5)c(C(F)F)cc43)nn2C2CCN(C(=O)CCCCCCCNc3ccc4c(c3)n(C)c(=O)n4C3CCC(=O)NC3=O)CC2)C1] | 3-(5-((8-(4-(5-acetyl-3-(7- (difluoromethyl)-6-(1-methyl-1H- pyrazol-4-yl)-3,4-dihydroquinolin- 1(2H)-yl)-4,5,6,7-tetrahydro-1H- pyrazolo[4,3-c]pyridin-1- yl)piperidin-1-yl)-8- oxooctyl)amino)-3-methyl-2-oxo- 2,3-dihydro-1H-benzo[d]imidazol- 1-yl)piperidine-2,6-dione |
| P-162 | 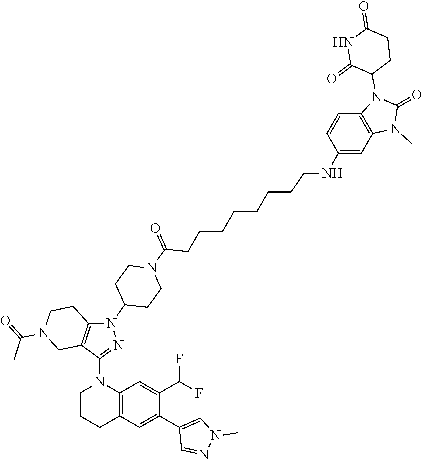 [SMILES: CC(=O)N1CCc2c(c(N3CCCc4cc(-c5cnn(C)c5)c(C(F)F)cc43)nn2C2CCN(C(=O)CCCCCCCCNc3ccc4c(c3)n(C)c(=O)n4C3CCC(=O)NC3=O)CC2)C1] | 3-(5-((9-(4-(5-acetyl-3-(7- (difluoromethyl)-6-(1-methyl-1H- pyrazol-4-yl)-3,4-dihydroquinolin- 1(2H)-yl)-4,5,6,7-tetrahydro-1H- pyrazolo[4,3-c]pyridin-1- yl)piperidin-1-yl)-9- oxononyl)amino)-3-methyl-2-oxo- 2,3-dihydro-1H-benzo[d]imidazol- 1-yl)piperidine-2,6-dione |
| P-163 | 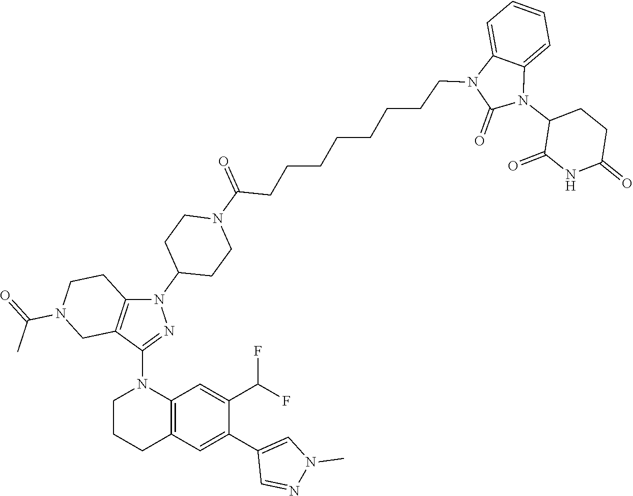 [SMILES: CC(=O)N1CCc2c(c(N3CCCc4cc(-c5cnn(C)c5)c(C(F)F)cc43)nn2C2CCN(C(=O)CCCCCCCCn3c(=O)n(C4CCC(=O)NC4=O)c4ccccc43)CC2)C1] | 3-(3-(9-(4-(5-acetyl-3-(7- (difluoromethyl)-6-(1-methyl-1H- pyrazol-4-yl)-3,4-dihydroquinolin- 1(2H)-yl)-4,5,6,7-tetrahydro-1H- pyrazolo[4,3-c]pyridin-1- yl)piperidin-1-yl)-9-oxononyl)-2- oxo-2,3-dihydro-1H- benzo[d]imidazol-1-yl)piperidine- 2,6-dione |
| P-164 | 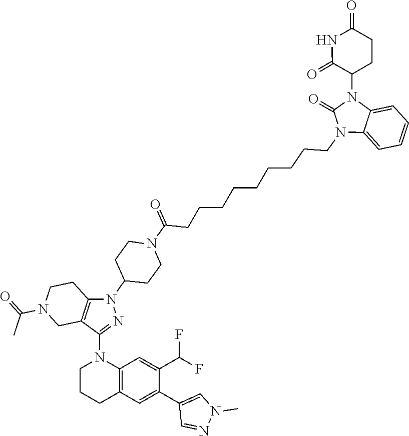 [SMILES: CC(=O)N1CCc2c(c(N3CCCc4cc(-c5cnn(C)c5)c(C(F)F)cc43)nn2C2CCN(C(=O)CCCCCCCCCn3c(=O)n(C4CCC(=O)NC4=O)c4ccccc43)CC2)C1] | 3-(3-(10-(4-(5-acetyl-3-(7- (difluoromethyl)-6-(1-methyl-1H- pyrazol-4-yl)-3,4-dihydroquinolin- 1(2H)-yl)-4,5,6,7-tetrahydro-1H- pyrazolo[4,3-c]pyridin-1- yl)piperidin-1-yl)-10-oxodecyl)-2- oxo-2,3-dihydro-1H- benzo[d]imidazol-1-yl)piperidine- 2,6-dione |
| P-165 | 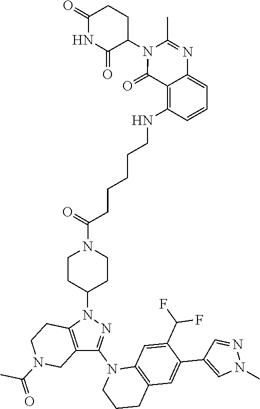 [SMILES: CC(=O)N1CCc2c(c(N3CCCc4cc(-c5cnn(C)c5)c(C(F)F)cc43)nn2C2CCN(C(=O)CCCCCNc3cccc4nc(C)n(C5CCC(=O)NC5=O)c(=O)c34)CC2)C1] | 3-(5-((6-(4-(5-acetyl-3-(7- (difluoromethyl)-6-(1-methyl-1H- pyrazol-4-yl)-3,4-dihydroquinolin- 1(2H)-yl)-4,5,6,7-tetrahydro-1H- pyrazolo[4,3-c]pyridin-1- yl)piperidin-1-yl)-6- oxohexyl)amino)-2-methyl-4- oxoquinazolin-3(4H)-yl)piperidine- 2,6-dione |
| P-166 | 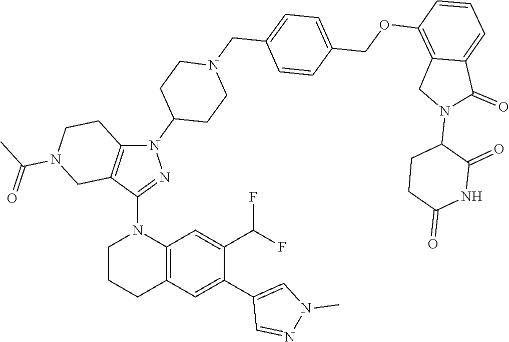 [SMILES: CC(=O)N1CCc2c(c(N3CCCc4cc(-c5cnn(C)c5)c(C(F)F)cc43)nn2C2CCN(Cc3ccc(COc4cccc5c4CN(C4CCC(=O)NC4=O)C5=O)cc3)CC2)C1] | 3-(4-((4-((4-(5-acetyl-3-(7- (difluoromethyl)-6-(1-methyl-1H- pyrazol-4-yl)-3,4-dihydroquinolin- 1(2H)-yl)-4,5,6,7-tetrahydro-1H- pyrazolo[4,3-c]pyridin-1- yl)piperidin-1- yl)methyl)benzyl)oxy)-1- oxoisoindolin-2-yl)piperidine-2,6- dione |
| P-167 | 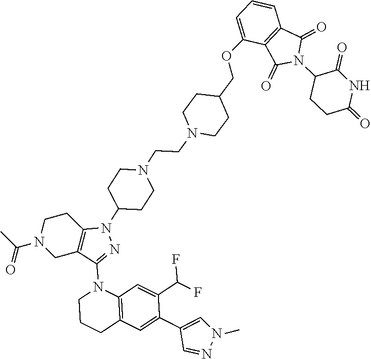 [SMILES: CC(=O)N1CCc2c(c(N3CCCc4cc(-c5cnn(C)c5)c(C(F)F)cc43)nn2C2CCN(CCN3CCC(COc4cccc5c4C(=O)N(C4CCC(=O)NC4=O)C5=O)CC3)CC2)C1] | 4-((1-(2-(4-(5-acetyl-3-(7- (difluoromethyl)-6-(1-methyl-1H- pyrazol-4-yl)-3,4-dihydroquinolin- 1(2H)-yl)-4,5,6,7-tetrahydro-1H- pyrazolo[4,3-c]pyridin-1- yl)piperidin-1-yl)ethyl)piperidin-4- yl)methoxy)-2-(2,6-dioxopiperidin- 3-yl)isoindoline-1,3-dione |
| P-168 | 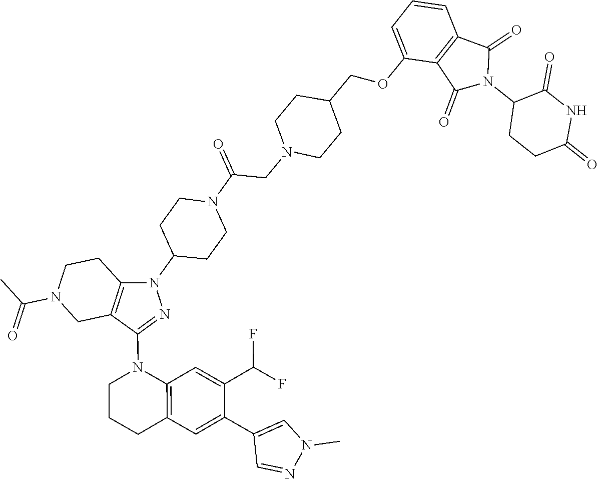 [SMILES: CC(=O)N1CCc2c(c(N3CCCc4cc(-c5cnn(C)c5)c(C(F)F)cc43)nn2C2CCN(C(=O)CN3CCC(COc4cccc5c4C(=O)N(C4CCC(=O)NC4=O)C5=O)CC3)CC2)C1] | 4-((1-(2-(4-(5-acetyl-3-(7- (difluoromethyl)-6-(1-methyl-1H- pyrazol-4-yl)-3,4-dihydroquinolin- 1(2H)-yl)-4,5,6,7-tetrahydro-1H- pyrazolo[4,3-c]pyridin-1- yl)piperidin-1-yl)-2- oxoethyl)piperidin-4-yl)methoxy)-2- (2,6-dioxopiperidin-3- yl)isoindoline-1,3-dione |
| P-169 | 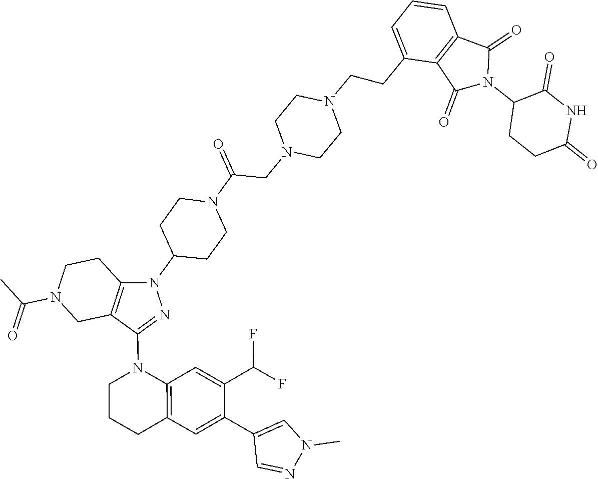 [SMILES: CC(=O)N1CCc2c(c(N3CCCc4cc(-c5cnn(C)c5)c(C(F)F)cc43)nn2C2CCN(C(=O)CN3CCN(CCc4cccc5c4C(=O)N(C4CCC(=O)NC4=O)C5=O)CC3)CC2)C1] | 4-(2-(4-(2-(4-(5-acetyl-3-(7- (difluoromethyl)-6-(1-methyl-1H- pyrazol-4-yl)-3,4-dihydroquinolin- 1(2H)-yl)-4,5,6,7-tetrahydro-1H- pyrazolo[4,3-c]pyridin-1- yl)piperidin-1-yl)-2- oxoethyl)piperazin-1-yl)ethyl)-2- (2,6-dioxopiperidin-3- yl)isoindoline-1,3-dione |
| P-170 | 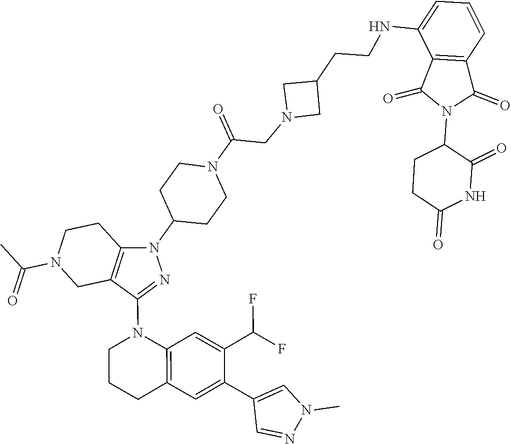 [SMILES: CC(=O)N1CCc2c(c(N3CCCc4cc(-c5cnn(C)c5)c(C(F)F)cc43)nn2C2CCN(C(=O)CN3CC(CCNc4cccc5c4C(=O)N(C4CCC(=O)NC4=O)C5=O)C3)CC2)C1] | 4-((2-(1-(2-(4-(5-acetyl-3-(7- (difluoromethyl)-6-(1-methyl-1H- pyrazol-4-yl)-3,4-dihydroquinolin- 1(2H)-yl)-4,5,6,7-tetrahydro-1H- pyrazolo[4,3-c]pyridin-1- yl)piperidin-1-yl)-2- oxoethyl)azetidin-3- yl)ethyl)amino)-2-(2,6- dioxopiperidin-3-yl)isoindoline-1,3- dione |
| P-171 | 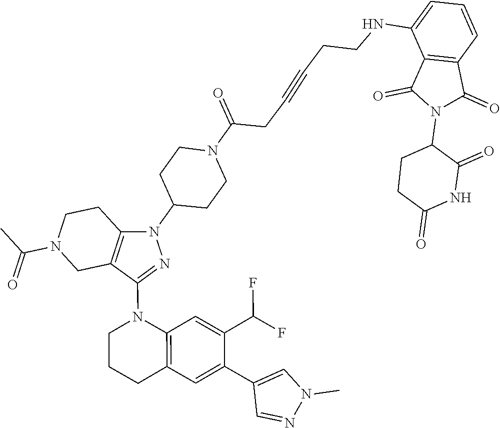 [SMILES: CC(=O)N1CCc2c(c(N3CCCc4cc(-c5cnn(C)c5)c(C(F)F)cc43)nn2C2CCN(C(=O)CC#CCCNc3cccc4c3C(=O)N(C3CCC(=O)NC3=O)C4=O)CC2)C1] | 4-((6-(4-(5-acetyl-3-(7- (difluoromethyl)-6-(1-methyl-1H- pyrazol-4-yl)-3,4-dihydroquinolin- 1(2H)-yl)-4,5,6,7-tetrahydro-1H- pyrazolo[4,3-c]pyridin-1- yl)piperidin-1-yl)-6-oxohex-3-yn-1- yl)amino)-2-(2,6-dioxopiperidin-3- yl)isoindoline-1,3-dione |
| P-172 | 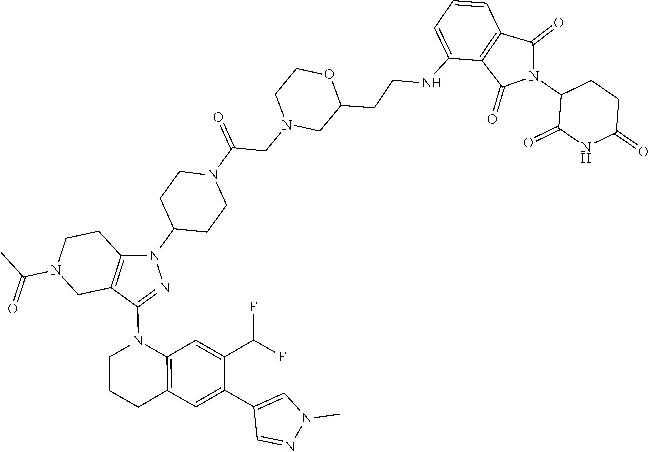 [SMILES: CC(=O)N1CCc2c(c(N3CCCc4cc(-c5cnn(C)c5)c(C(F)F)cc43)nn2C2CCN(C(=O)CN3CCOC(CCNc4cccc5c4C(=O)N(C4CCC(=O)NC4=O)C5=O)C3)CC2)C1] | 4-((2-(4-(2-(4-(5-acetyl-3-(7- (difluoromethyl)-6-(1-methyl-1H- pyrazol-4-yl)-3,4-dihydroquinolin- 1(2H)-yl)-4,5,6,7-tetrahydro-1H- pyrazolo[4,3-c]pyridin-1- yl)piperidin-1-yl)-2- oxoethyl)morpholin-2- yl)ethyl)amino)-2-(2,6- dioxopiperidin-3-yl)isoindoline-1,3- dione |
| P-173 | 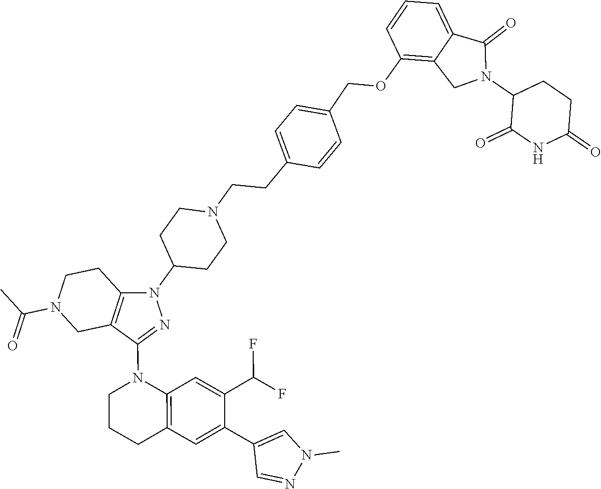 [SMILES: CC(=O)N1CCc2c(c(N3CCCc4cc(-c5cnn(C)c5)c(C(F)F)cc43)nn2C2CCN(CCc3ccc(COc4cccc5c4CN(C4CCC(=O)NC4=O)C5=O)cc3)CC2)C1] | 3-(4-((4-(2-(4-(5-acetyl-3-(7- (difluoromethyl)-6-(1-methyl-1H- pyrazol-4-yl)-3,4-dihydroquinolin- 1(2H)-yl)-4,5,6,7-tetrahydro-1H- pyrazolo[4,3-c]pyridin-1- yl)piperidin-1-yl)ethyl)benzyl)oxy)- 1-oxoisoindolin-2-yl)piperidine-2,6- dione |
| P-174 | 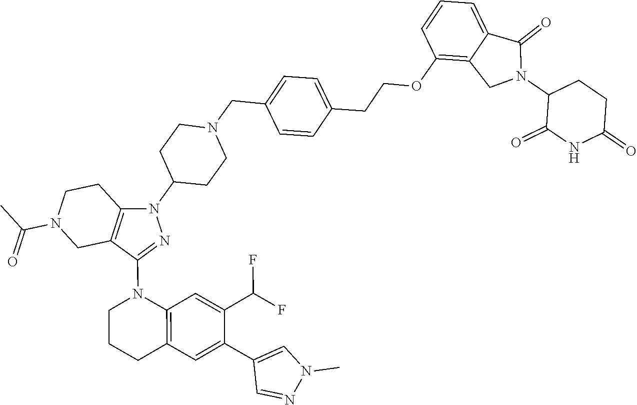 [SMILES: CC(=O)N1CCc2c(c(N3CCCc4cc(-c5cnn(C)c5)c(C(F)F)cc43)nn2C2CCN(Cc3ccc(CCOc4cccc5c4CN(C4CCC(=O)NC4=O)C5=O)cc3)CC2)C1] | 3-(4-(4-((4-(5-acetyl-3-(7- (difluoromethyl)-6-(1-methyl-1H- pyrazol-4-yl)-3,4-dihydroquinolin- 1(2H)-yl)-4,5,6,7-tetrahydro-1H- pyrazolo[4,3-c]pyridin-1- yl)piperidin-1- yl)methyl)phenethoxy)-1- oxoisoindolin-2-yl)piperidine-2,6- dione |
| CPD- 1139 | 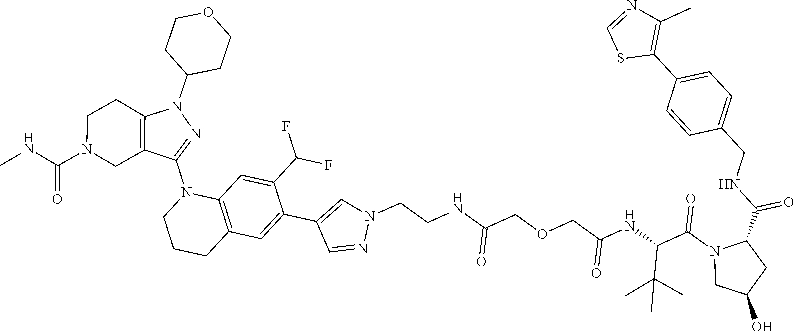 [SMILES: CNC(=O)N1CCc2c(c(N3CCCc4cc(-c5cnn(CCNC(=O)COCC(=O)N[C@H](C(=O)N6C[C@H](O)C[C@H]6C(=O)NCc6ccc(-c7scnc7C)cc6)C(C)(C)C)c5)c(C(F)F)cc43)nn2C2CCOCC2)C1] | 3-(7-(difluoromethyl)-6-(1-(2-(2-(2- (((S)-1-((2S,4R)-4-hydroxy-2-((4-(4- methylthiazol-5- yl)benzyl)carbamoyl)pyrrolidin-1- yl)-3,3-dimethyl-1-oxobutan-2- yl)amino)-2- oxoethoxy)acetamido)ethyl)-1H- pyrazol-4-yl)-3,4-dihydroquinolin- 1(2H)-yl)-N-methyl-1-(tetrahydro- 2H-pyran-4-yl)-1,4,6,7-tetrahydro- 5H-pyrazolo[4,3-c]pyridine-5- carboxamide |
| CPD- 1140 | 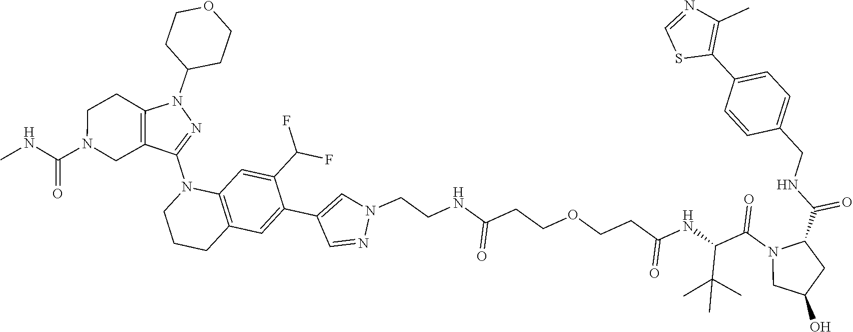 [SMILES: CNC(=O)N1CCc2c(c(N3CCCc4cc(-c5cnn(CCNC(=O)CCOCCC(=O)N[C@H](C(=O)N6C[C@H](O)C[C@H]6C(=O)NCc6ccc(-c7scnc7C)cc6)C(C)(C)C)c5)c(C(F)F)cc43)nn2C2CCOCC2)C1] | 3-(7-(difluoromethyl)-6-(1-(2-(3-(3- (((S)-1-((2S,4R)-4-hydroxy-2-((4-(4- methylthiazol-5- yl)benzyl)carbamoyl)pyrrolidin-1- yl)-3,3-dimethyl-1-oxobutan-2- yl)amino)-3- oxopropoxy)propanamido)ethyl)- 1H-pyrazol-4-yl)-3,4- dihydroquinolin-1(2H)-yl)-N- methyl-1-(tetrahydro-2H-pyran-4- yl)-1,4,6,7-tetrahydro-5H- pyrazolo[4,3-c]pyridine-5- carboxamide |
| CPD- 1141 | 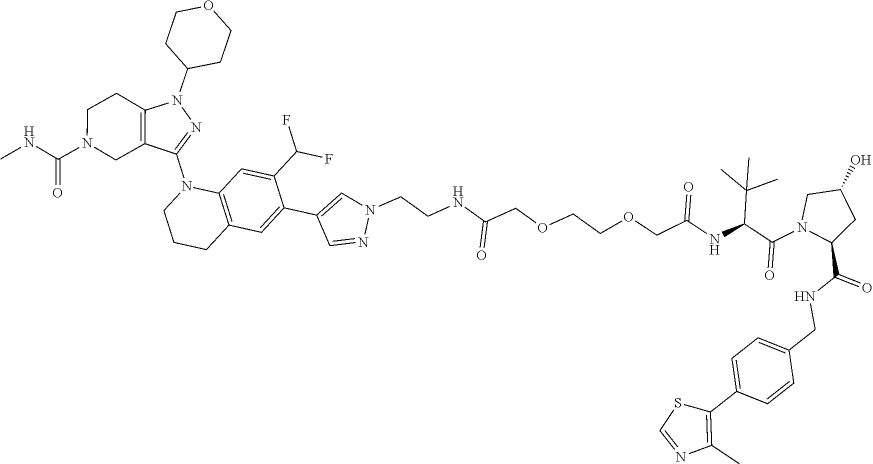 [SMILES: CNC(=O)N1CCc2c(c(N3CCCc4cc(-c5cnn(CCNC(=O)COCCOCC(=O)N[C@H](C(=O)N6C[C@H](O)C[C@H]6C(=O)NCc6ccc(-c7scnc7C)cc6)C(C)(C)C)c5)c(C(F)F)cc43)nn2C2CCOCC2)C1] | 3-(7-(difluoromethyl)-6-(1-((S)-13- ((2S,4R)-4-hydroxy-2-((4-(4- methylthiazol-5- yl)benzyl)carbamoyl)pyrrolidine-1- carbonyl)-14,14-dimethyl-4,11- dioxo-6,9-dioxa-3,12- diazapentadecyl)-1H-pyrazol-4-yl)- 3,4-dihydroquinolin- methyl-1-(tetrahydro-2H-pyran-4- yl)-1,4,6,7-tetrahydro-5H- pyrazolo[4,3-c]pyridine-5- carboxamide |
| CPD- 1142 | 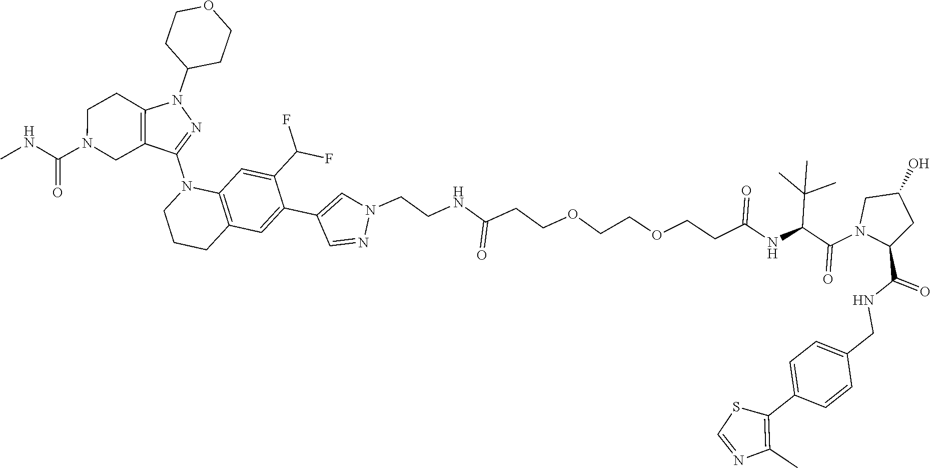 [SMILES: CNC(=O)N1CCc2c(c(N3CCCc4cc(-c5cnn(CCNC(=O)CCOCCOCCC(=O)N[C@H](C(=O)N6C[C@H](O)C[C@H]6C(=O)NCc6ccc(-c7scnc7C)cc6)C(C)(C)C)c5)c(C(F)F)cc43)nn2C2CCOCC2)C1] | 3-(7-(difluoromethyl)-6-(1-((S)-15- ((2S,4R)-4-hydroxy-2-((4-(4- methylthiazol-5- yl)benzyl)carbamoyl)pyrrolidine-1- carbonyl)-16,16-dimethyl-4,13- dioxo-7,10-dioxa-3,14- diazaheptadecyl)-1H-pyrazol-4-yl)- 3,4-dihydroquinolin- methyl-1-(tetrahydro-2H-pyran-4- yl)-1,4,6,7-tetrahydro-5H- pyrazolo[4,3-c]pyridine-5- carboxamide |
| CPD- 1143 | 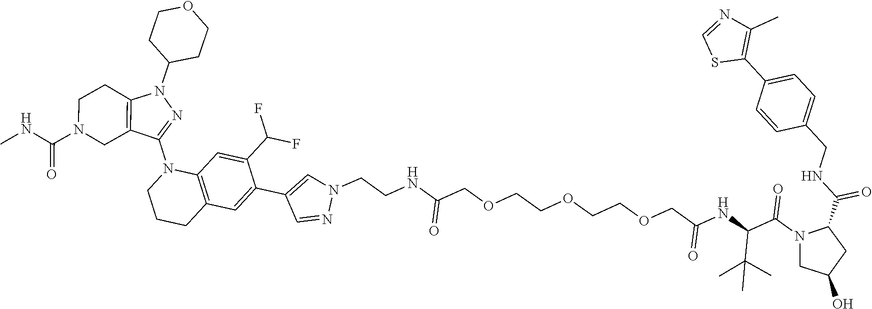 [SMILES: CNC(=O)N1CCc2c(c(N3CCCc4cc(-c5cnn(CCNC(=O)COCCOCCOCC(=O)N[C@@H](C(=O)N6C[C@H](O)C[C@H]6C(=O)NCc6ccc(-c7scnc7C)cc6)C(C)(C)C)c5)c(C(F)F)cc43)nn2C2CCOCC2)C1] | 3-(7-(difluoromethyl)-6-(1-((S)-16- ((2S,4R)-4-hydroxy-2-((4-(4- methylthiazol-5- yl)benzyl)carbamoyl)pyrrolidine-1- carbonyl)-17,17-dimethyl-4,14- dioxo-6,9,12-trioxa-3,15- diazaoctadecyl)-1H-pyrazol-4-yl)- 3,4-dihydroquinolin-1(2H)-yl)-N- methyl-1-(tetrahydro-2H-pyran-4- yl)-1,4,6,7-tetrahydro-5H- pyrazolo[4,3-c]pyridine-5- carboxamide |
| CPD- 1144 | 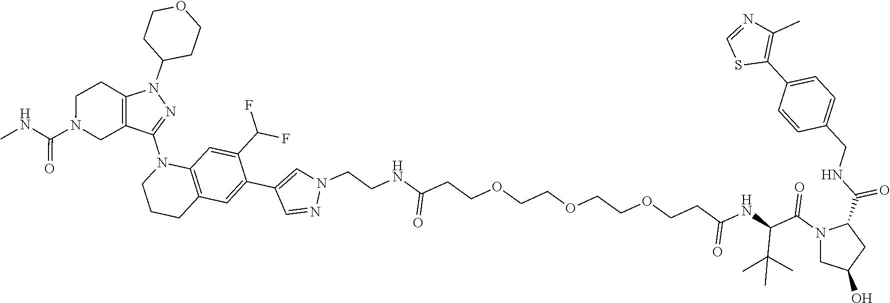 [SMILES: CNC(=O)N1CCc2c(c(N3CCCc4cc(-c5cnn(CCNC(=O)CCOCCOCCOCCC(=O)N[C@@H](C(=O)N6C[C@H](O)C[C@H]6C(=O)NCc6ccc(-c7scnc7C)cc6)C(C)(C)C)c5)c(C(F)F)cc43)nn2C2CCOCC2)C1] | 3-(7-(difluoromethyl)-6-(1-((S)-18- ((2S,4R)-4-hydroxy-2-((4-(4- methylthiazol-5- yl)benzyl)carbamoyl)pyrrolidine-1- carbonyl)-19,19-dimethyl-4,16- dioxo-7,10,13-trioxa-3,17- diazaicosyl)-1H-pyrazol-4-yl)-3,4- dihydroquinolin-1(2H)-yl)-N- methyl-1-(tetrahydro-2H-pyran-4- yl)-1,4,6,7-tetrahydro-5H- pyrazolo[4,3-c]pyridine-5- carboxamide |
| CPD- 1145 | 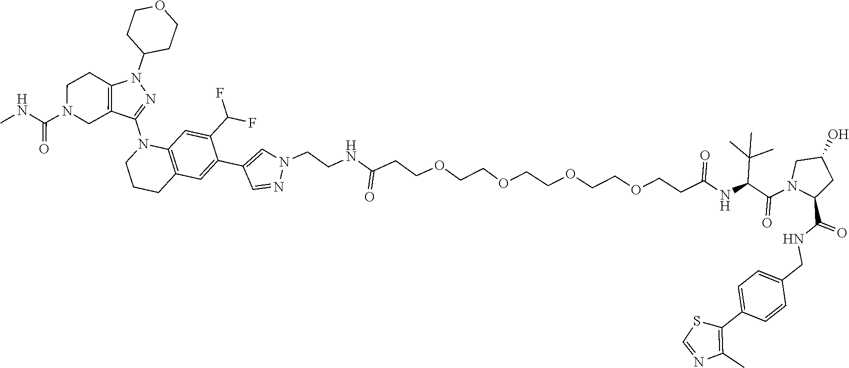 [SMILES: CNC(=O)N1CCc2c(c(N3CCCc4cc(-c5cnn(CCNC(=O)CCOCCOCCOCCOCCC(=O)N[C@H](C(=O)N6C[C@H](O)C[C@H]6C(=O)NCc6ccc(-c7scnc7C)cc6)C(C)(C)C)c5)c(C(F)F)cc43)nn2C2CCOCC2)C1] | N1-(2-(4-(7-(difluoromethyl)-1-(5- (methylcarbamoyl)-1-(tetrahydro- 2H-pyran-4-yl)-4,5,6,7-tetrahydro- 1H-pyrazolo[4,3-c]pyridin-3-yl)- 1,2,3,4-tetrahydroquinolin-6-yl)-1H- pyrazol-1-yl)ethyl)-N16-((S)-1- ((2S,4R)-4-hydroxy-2-((4-(4- methylthiazol-5- yl)benzyl)carbamoyl)pyrrolidin-1- yl)-3,3-dimethyl-1-oxobutan-2-yl)- 4,7,10,13- tetraoxahexadecanediamide |
| CPD- 1146 | 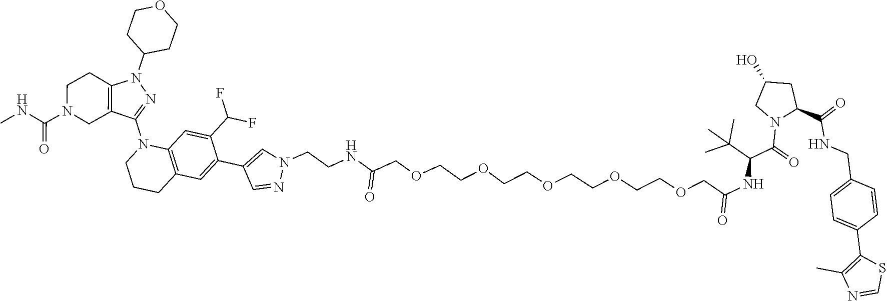 [SMILES: CNC(=O)N1CCc2c(c(N3CCCc4cc(-c5cnn(CCNC(=O)COCCOCCOCCOCCOCC(=O)N[C@H](C(=O)N6C[C@H](O)C[C@H]6C(=O)NCc6ccc(-c7scnc7C)cc6)C(C)(C)C)c5)c(C(F)F)cc43)nn2C2CCOCC2)C1] | N1-(2-(4-(7-(difluoromethyl)-1-(5- (methylcarbamoyl)-1-(tetrahydro- 2H-pyran-4-yl)-4,5,6,7-tetrahydro- 1H-pyrazolo[4,3-c]pyridin-3-yl)- 1,2,3,4-tetrahydroquinolin-6-yl)-1H- pyrazol-1-yl)ethyl)-N17-((S)-1- ((2S,4R)-4-hydroxy-2-((4-(4- methylthiazol-5- yl)benzyl)carbamoyl)pyrrolidin-1- yl)-3,3-dimethyl-1-oxobutan-2-yl)- 3,6,9,12,15- pentaoxaheptadecanediamide |
| CPD- 1147 | 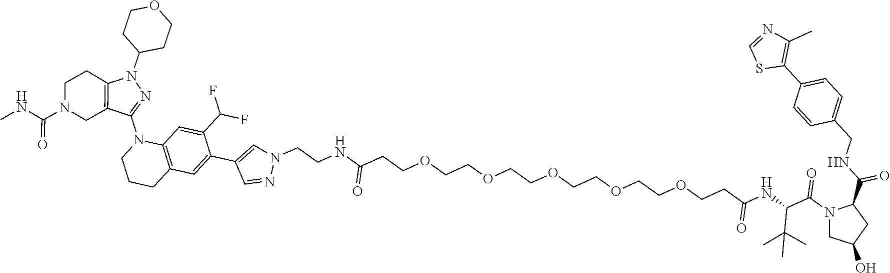 [SMILES: CNC(=O)N1CCc2c(c(N3CCCc4cc(-c5cnn(CCNC(=O)CCOCCOCCOCCOCCOCCC(=O)N[C@H](C(=O)N6C[C@H](O)C[C@@H]6C(=O)NCc6ccc(-c7scnc7C)cc6)C(C)(C)C)c5)c(C(F)F)cc43)nn2C2CCOCC2)C1] | N1-(2-(4-(7-(difluoromethyl)-1-(5- (methylcarbamoyl)-1-(tetrahydro- 2H-pyran-4-yl)-4,5,6,7-tetrahydro- 1H-pyrazolo[4,3-c]pyridin-3-yl)- 1,2,3,4-tetrahydroquinolin-6-yl)-1H- pyrazol-1-yl)ethyl)-N19-((S)-1- ((2S,4R)-4-hydroxy-2-((4-(4- methylthiazol-5- yl)benzyl)carbamoyl)pyrrolidin-1- yl)-3,3-dimethyl-1-oxobutan-2-yl)- 4,7,10,13,16- pentaoxanonadecanediamide |
| CPD- 1148 | 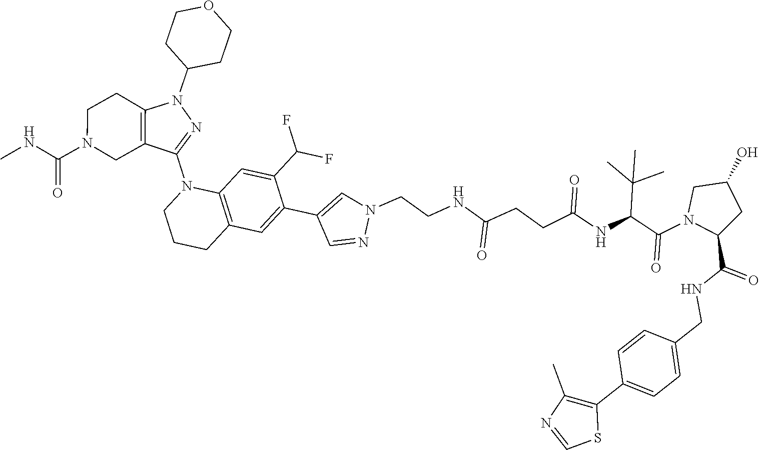 [SMILES: CNC(=O)N1CCc2c(c(N3CCCc4cc(-c5cnn(CCNC(=O)CCC(=O)N[C@H](C(=O)N6C[C@H](O)C[C@H]6C(=O)NCc6ccc(-c7scnc7C)cc6)C(C)(C)C)c5)c(C(F)F)cc43)nn2C2CCOCC2)C1] | N1-(2-(4-(7-(difluoromethyl)-1-(5- (methylcarbamoyl)-1-(tetrahydro- 2H-pyran-4-yl)-4,5,6,7-tetrahydro- 1H-pyrazolo[4,3-c]pyridin-3-yl)- 1,2,3,4-tetrahydroquinolin-6-yl)-1H- pyrazol-1-yl)ethyl)-N4-((S)-1- ((2S,4R)-4-hydroxy-2-((4-(4- methylthiazol-5- yl)benzyl)carbamoyl)pyrrolidin-1- yl)-3,3-dimethyl-1-oxobutan-2- yl)succinamide |
| CPD- 1149 | 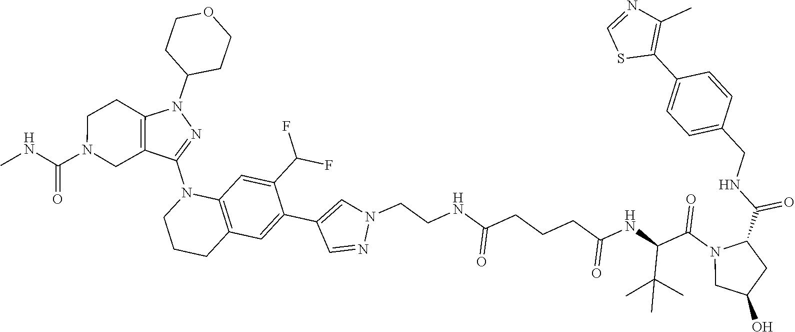 [SMILES: CNC(=O)N1CCc2c(c(N3CCCc4cc(-c5cnn(CCNC(=O)CCCC(=O)N[C@@H](C(=O)N6C[C@H](O)C[C@H]6C(=O)NCc6ccc(-c7scnc7C)cc6)C(C)(C)C)c5)c(C(F)F)cc43)nn2C2CCOCC2)C1] | N1-(2-(4-(7-(difluoromethyl)-1-(5- (methylcarbamoyl)-1-(tetrahydro- 2H-pyran-4-yl)-4,5,6,7-tetrahydro- 1H-pyrazolo[4,3-c]pyridin-3-yl)- 1,2,3,4-tetrahydroquinolin-6-yl)-1H- pyrazol-1-yl)ethyl)-N5-((S)-1- ((2S,4R)-4-hydroxy-2-((4-(4- methylthiazol-5- yl)benzyl)carbamoyl)pyrrolidin-1- yl)-3,3-dimethyl-1-oxobutan-2- yl)glutaramide |
| CPD- 1150 | 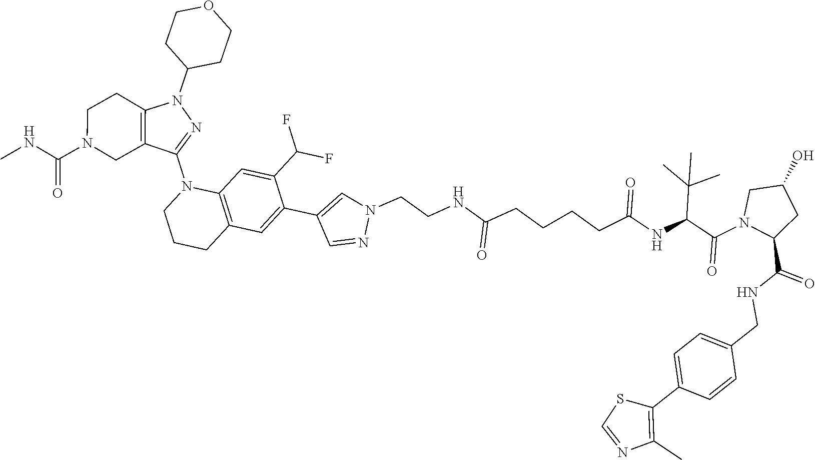 [SMILES: CNC(=O)N1CCc2c(c(N3CCCc4cc(-c5cnn(CCNC(=O)CCCCC(=O)N[C@H](C(=O)N6C[C@H](O)C[C@H]6C(=O)NCc6ccc(-c7scnc7C)cc6)C(C)(C)C)c5)c(C(F)F)cc43)nn2C2CCOCC2)C1] | N1-(2-(4-(7-(difluoromethyl)-1-(5- (methylcarbamoyl)-1-(tetrahydro- 2H-pyran-4-yl)-4,5,6,7-tetrahydro- 1H-pyrazolo[4,3-c]pyridin-3-yl)- 1,2,3,4-tetrahydroquinolin-6-yl)-1H- pyrazol-1-yl)ethyl)-N6-((S)-1- ((2S,4R)-4-hydroxy-2-((4-(4- methylthiazol-5- yl)benzyl)carbamoyl)pyrrolidin-1- yl)-3,3-dimethyl-1-oxobutan-2- yl)adipamide |
| CPD- 1151 | 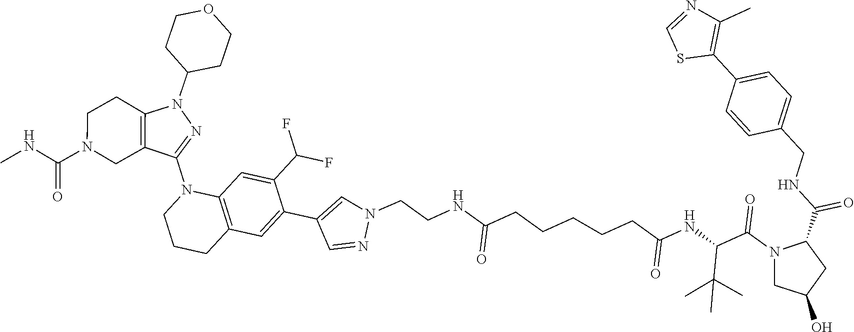 [SMILES: CNC(=O)N1CCc2c(c(N3CCCc4cc(-c5cnn(CCNC(=O)CCCCCC(=O)N[C@H](C(=O)N6C[C@H](O)C[C@H]6C(=O)NCc6ccc(-c7scnc7C)cc6)C(C)(C)C)c5)c(C(F)F)cc43)nn2C2CCOCC2)C1] | N1-(2-(4-(7-(difluoromethyl)-1-(5- (methylcarbamoyl)-1-(tetrahydro- 2H-pyran-4-yl)-4,5,6,7-tetrahydro- 1H-pyrazolo[4,3-c]pyridin-3-yl)- 1,2,3,4-tetrahydroquinolin-6-yl)-1H- pyrazol-1-yl)ethyl)-N7-((S)-1- ((2S,4R)-4-hydroxy-2-((4-(4- methylthiazol-5- yl)benzyl)carbamoyl)pyrrolidin-1- yl)-3,3-dimethyl-1-oxobutan-2- yl)heptanediamide |
| CPD- 1152 | 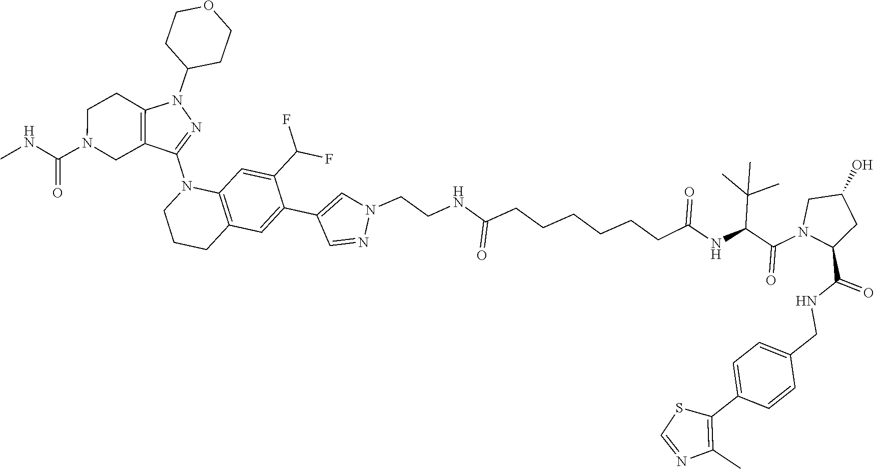 [SMILES: CNC(=O)N1CCc2c(c(N3CCCc4cc(-c5cnn(CCNC(=O)CCCCCCC(=O)N[C@H](C(=O)N6C[C@H](O)C[C@H]6C(=O)NCc6ccc(-c7scnc7C)cc6)C(C)(C)C)c5)c(C(F)F)cc43)nn2C2CCOCC2)C1] | N1-(2-(4-(7-(difluoromethyl)-1-(5- (methylcarbamoyl)-1-(tetrahydro- 2H-pyran-4-yl)-4,5,6,7-tetrahydro- 1H-pyrazolo[4,3-c]pyridin-3-yl)- 1,2,3,4-tetrahydroquinolin-6-yl)-1H- pyrazol-1-yl)ethyl)-N8-((S)-1- ((2S,4R)-4-hydroxy-2-((4-(4- methylthiazol-5- yl)benzyl)carbamoyl)pyrrolidin-1- yl)-3,3-dimethyl-1-oxobutan-2- yl)octanediamide |
| CPD- 1153 | 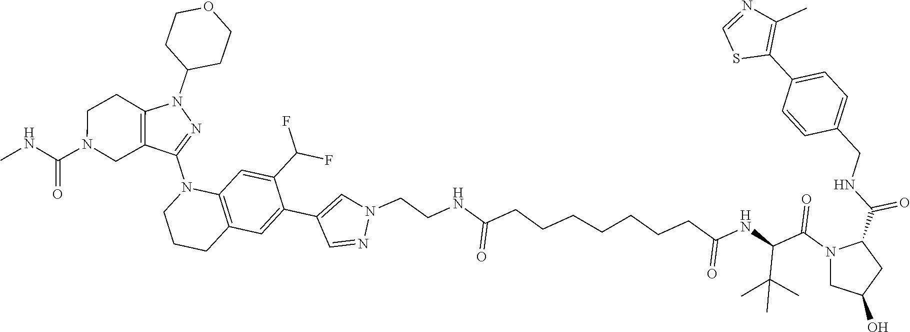 [SMILES: CNC(=O)N1CCc2c(c(N3CCCc4cc(-c5cnn(CCNC(=O)CCCCCCCC(=O)N[C@@H](C(=O)N6C[C@H](O)C[C@H]6C(=O)NCc6ccc(-c7scnc7C)cc6)C(C)(C)C)c5)c(C(F)F)cc43)nn2C2CCOCC2)C1] | N1-(2-(4-(7-(difluoromethyl)-1-(5- (methylcarbamoyl)-1-(tetrahydro- 2H-pyran-4-yl)-4,5,6,7-tetrahydro- 1H-pyrazolo[4,3-c]pyridin-3-yl)- 1,2,3,4-tetrahydroquinolin-6-yl)-1H- pyrazol-1-yl)ethyl)-N9-((S)-1- ((2S,4R)-4-hydroxy-2-((4-(4- methylthiazol-5- yl)benzyl)carbamoyl)pyrrolidin-1- yl)-3,3-dimethyl-1-oxobutan-2- yl)nonanediamide |
| CPD- 1154 | 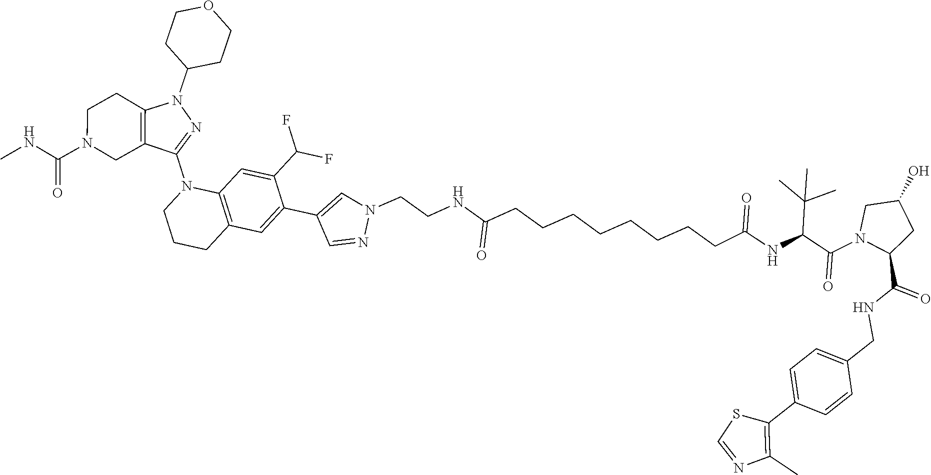 [SMILES: CNC(=O)N1CCc2c(c(N3CCCc4cc(-c5cnn(CCNC(=O)CCCCCCCCC(=O)N[C@H](C(=O)N6C[C@H](O)C[C@H]6C(=O)NCc6ccc(-c7scnc7C)cc6)C(C)(C)C)c5)c(C(F)F)cc43)nn2C2CCOCC2)C1] | N1-(2-(4-(7-(difluoromethyl)-1-(5- (methylcarbamoyl)-1-(tetrahydro- 2H-pyran-4-yl)-4,5,6,7-tetrahydro- 1H-pyrazolo[4,3-c]pyridin-3-yl)- 1,2,3,4-tetrahydroquinolin-6-yl)-1H- pyrazol-1-yl)ethyl)-N10-((S)-1- ((2S,4R)-4-hydroxy-2-((4-(4- methylthiazol-5- yl)benzyl)carbamoyl)pyrrolidin-1- yl)-3,3-dimethyl-1-oxobutan-2- yl)decanediamide |
| CPD- 1155 | 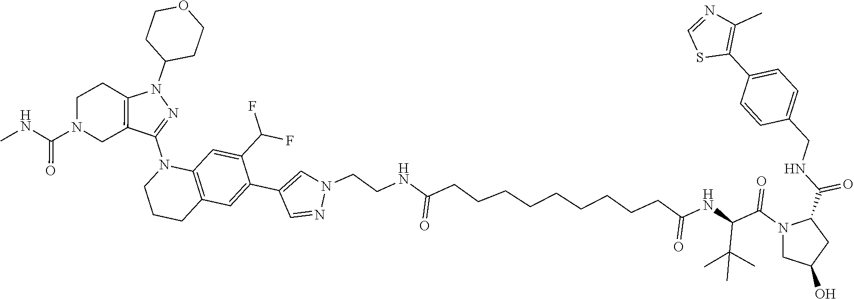 [SMILES: CNC(=O)N1CCc2c(c(N3CCCc4cc(-c5cnn(CCNC(=O)CCCCCCCCCC(=O)N[C@@H](C(=O)N6C[C@H](O)C[C@H]6C(=O)NCc6ccc(-c7scnc7C)cc6)C(C)(C)C)c5)c(C(F)F)cc43)nn2C2CCOCC2)C1] | N1-(2-(4-(7-(difluoromethyl)-1-(5- (methylcarbamoyl)-1-(tetrahydro- 2H-pyran-4-yl)-4,5,6,7-tetrahydro- 1H-pyrazolo[4,3-c]pyridin-3-yl)- 1,2,3,4-tetrahydroquinolin-6-yl)-1H- pyrazol-1-yl)ethyl)-N11-((S)-1- ((2S,4R)-4-hydroxy-2-((4-(4- methylthiazol-5- yl)benzyl)carbamoyl)pyrrolidin-1- yl)-3,3-dimethyl-1-oxobutan-2- yl)undecanediamide |
| CPD- 1156 | 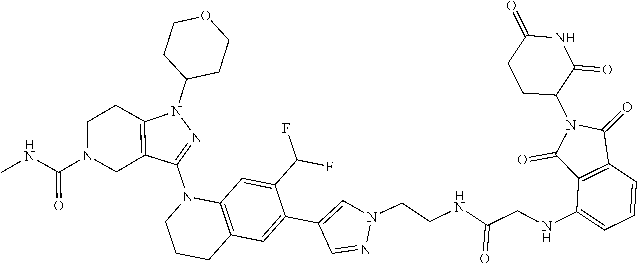 [SMILES: CNC(=O)N1CCc2c(c(N3CCCc4cc(-c5cnn(CCNC(=O)CNc6cccc7c6C(=O)N(C6CCC(=O)NC6=O)C7=O)c5)c(C(F)F)cc43)nn2C2CCOCC2)C1] | 3-(7-(difluoromethyl)-6-(1-(2-(2- ((2-(2,6-dioxopiperidin-3-yl)-1,3- dioxoisoindolin-4- yl)amino)acetamido)ethyl)-1H- pyrazol-4-yl)-3,4-dihydroquinolin- 1(2H)-yl)-N-methyl-1-(tetrahydro- 2H-pyran-4-yl)-1,4,6,7-tetrahydro- 5H-pyrazolo[4,3-c]pyridine-5- carboxamide |
| CPD- 1157 | 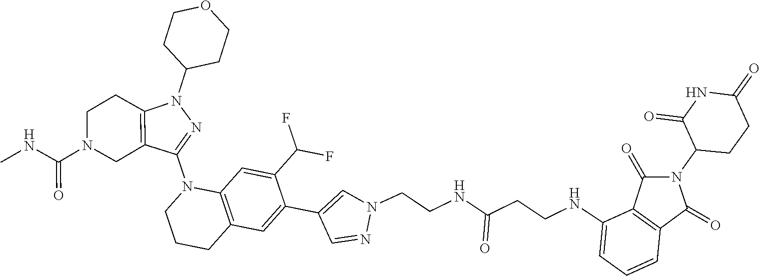 [SMILES: CNC(=O)N1CCc2c(c(N3CCCc4cc(-c5cnn(CCNC(=O)CCNc6cccc7c6C(=O)N(C6CCC(=O)NC6=O)C7=O)c5)c(C(F)F)cc43)nn2C2CCOCC2)C1] | 3-(7-(difluoromethyl)-6-(1-(2-(3- ((2-(2,6-dioxopiperidin-3-yl)-1,3- dioxoisoindolin-4- yl)amino)propanamido)ethyl)-1H- pyrazol-4-yl)-3,4-dihydroquinolin- 1(2H)-yl)-N-methyl-1-(tetrahydro- 2H-pyran-4-yl)-1,4,6,7-tetrahydro- 5H-pyrazolo[4,3-c]pyridine-5- carboxamide |
| CPD- 1158 | 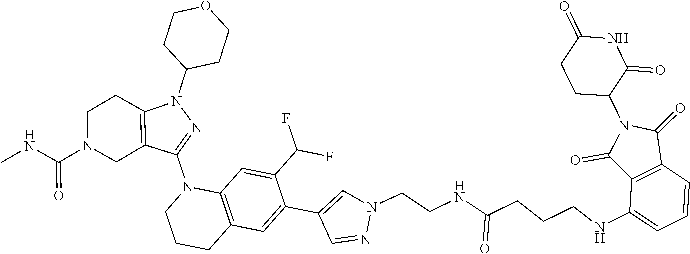 [SMILES: CNC(=O)N1CCc2c(c(N3CCCc4cc(-c5cnn(CCNC(=O)CCCNc6cccc7c6C(=O)N(C6CCC(=O)NC6=O)C7=O)c5)c(C(F)F)cc43)nn2C2CCOCC2)C1] | 3-(7-(difluoromethyl)-6-(1-(2-(4- ((2-(2,6-dioxopiperidin-3-yl)-1,3- dioxoisoindolin-4- yl)amino)butanamido)ethyl)-1H- pyrazol-4-yl)-3,4-dihydroquinolin- 1(2H)-yl)-N-methyl-1-(tetrahydro- 2H-pyran-4-yl)-1,4,6,7-tetrahydro- 5H-pyrazolo[4,3-c]pyridine-5- carboxamide |
| CPD- 1159 | 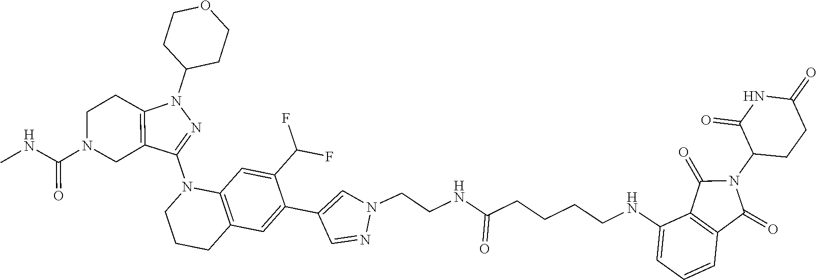 [SMILES: CNC(=O)N1CCc2c(c(N3CCCc4cc(-c5cnn(CCNC(=O)CCCCNc6cccc7c6C(=O)N(C6CCC(=O)NC6=O)C7=O)c5)c(C(F)F)cc43)nn2C2CCOCC2)C1] | 3-(7-(difluoromethyl)-6-(1-(2-(5- ((2-(2,6-dioxopiperidin-3-yl)-1,3- dioxoisoindolin-4- yl)amino)pentanamido)ethyl)-1H- pyrazol-4-yl)-3,4-dihydroquinolin- 1(2H)-yl)-N-methyl-1-(tetrahydro- 2H-pyran-4-yl)-1,4,6,7-tetrahydro- 5H-pyrazolo[4,3-c]pyridine-5- carboxamide |
| CPD- 1160 | 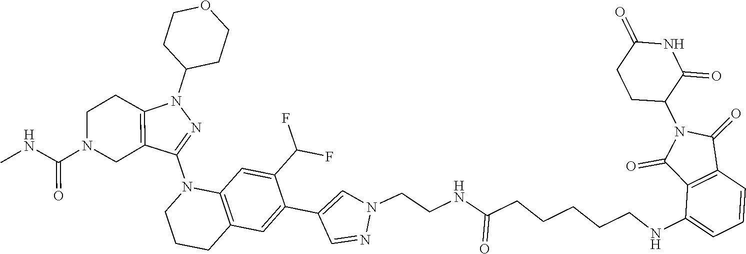 [SMILES: CNC(=O)N1CCc2c(c(N3CCCc4cc(-c5cnn(CCNC(=O)CCCCCNc6cccc7c6C(=O)N(C6CCC(=O)NC6=O)C7=O)c5)c(C(F)F)cc43)nn2C2CCOCC2)C1] | 3-(7-(difluoromethyl)-6-(1-(2-(6- ((2-(2,6-dioxopiperidin-3-yl)-1,3- dioxoisoindolin-4- yl)amino)hexanamido)ethyl)-1H- pyrazol-4-yl)-3,4-dihydroquinolin- 1(2H)-yl)-N-methyl-1-(tetrahydro- 2H-pyran-4-yl)-1,4,6,7-tetrahydro- 5H-pyrazolo[4,3-c]pyridine-5- carboxamide |
| CPD- 1161 | 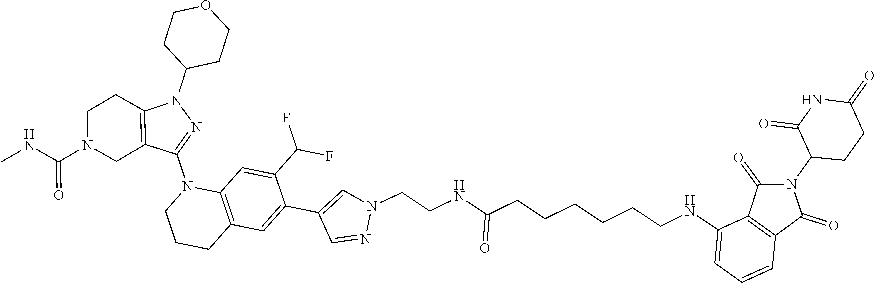 [SMILES: CNC(=O)N1CCc2c(c(N3CCCc4cc(-c5cnn(CCNC(=O)CCCCCCNc6cccc7c6C(=O)N(C6CCC(=O)NC6=O)C7=O)c5)c(C(F)F)cc43)nn2C2CCOCC2)C1] | 3-(7-(difluoromethyl)-6-(1-(2-(7- ((2-(2,6-dioxopiperidin-3-yl)-1,3- dioxoisoindolin-4- yl)amino)heptanamido)ethyl)-1H- pyrazol-4-yl)-3,4-dihydroquinolin- 1(2H)-yl)-N-methyl-1-(tetrahydro- 2H-pyran-4-yl)-1,4,6,7-tetrahydro- 5H-pyrazolo[4,3-c]pyridine-5- carboxamide |
| CPD- 1162 | 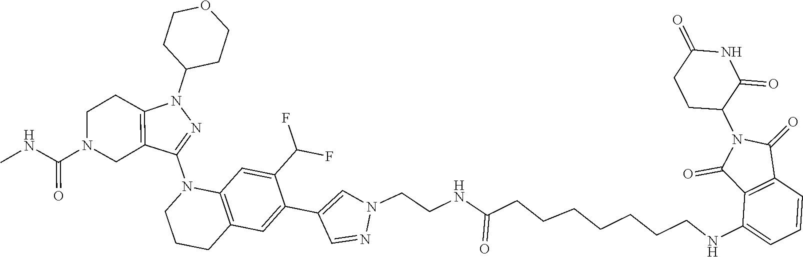 [SMILES: CNC(=O)N1CCc2c(c(N3CCCc4cc(-c5cnn(CCNC(=O)CCCCCCCNc6cccc7c6C(=O)N(C6CCC(=O)NC6=O)C7=O)c5)c(C(F)F)cc43)nn2C2CCOCC2)C1] | 3-(7-(difluoromethyl)-6-(1-(2-(8- ((2-(2,6-dioxopiperidin-3-yl)-1,3- dioxoisoindolin-4- yl)amino)octanamido)ethyl)-1H- pyrazol-4-yl)-3,4-dihydroquinolin- 1(2H)-yl)-N-methyl-1-(tetrahydro- 2H-pyran-4-yl)-1,4,6,7-tetrahydro- 5H-pyrazolo[4,3-c]pyridine-5- carboxamide |
| CPD- 1163 | 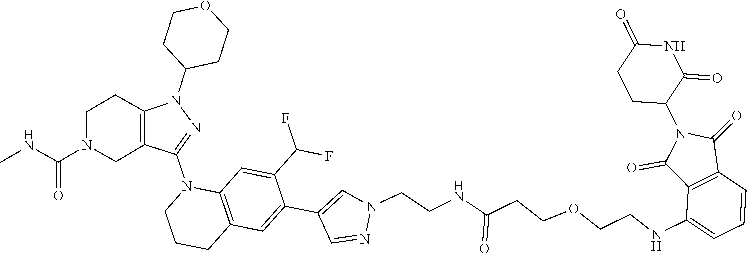 [SMILES: CNC(=O)N1CCc2c(c(N3CCCc4cc(-c5cnn(CCNC(=O)CCOCCNc6cccc7c6C(=O)N(C6CCC(=O)NC6=O)C7=O)c5)c(C(F)F)cc43)nn2C2CCOCC2)C1] | 3-(7-(difluoromethyl)-6-(1-(2-(3-(2- ((2-(2,6-dioxopiperidin-3-yl)-1,3- dioxoisoindolin-4- yl)amino)ethoxy)propanamido) ethyl)-1H-pyrazol-4-yl)-3,4- dihydroquinolin-1(2H)-yl)-N- methyl-1-(tetrahydro-2H-pyran-4- yl)-1,4,6,7-tetrahydro-5H- pyrazolo[4,3-c]pyridine-5- carboxamide |
| CPD- 1164 | 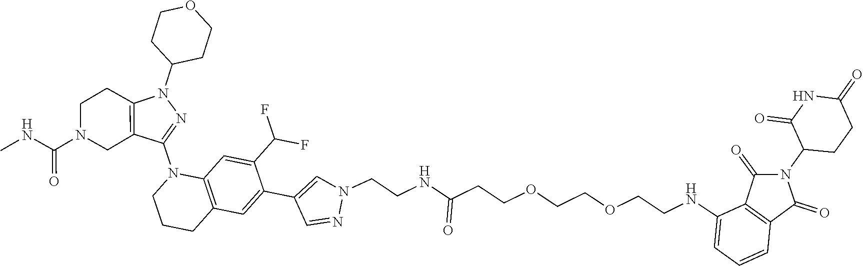 [SMILES: CNC(=O)N1CCc2c(c(N3CCCc4cc(-c5cnn(CCNC(=O)CCOCCOCCNc6cccc7c6C(=O)N(C6CCC(=O)NC6=O)C7=O)c5)c(C(F)F)cc43)nn2C2CCOCC2)C1] | 3-(7-(difluoromethyl)-6-(1-(2-(3-(2- (2-((2-(2,6-dioxopiperidin-3-yl)-1,3- dioxoisoindolin-4-yl) amino)ethoxy)ethoxy)propanamido) ethyl)-1H-pyrazol-4-yl)-3,4- dihydroquinolin-1(2H)-yl)-N- methyl-1-(tetrahydro-2H-pyran-4- yl)-1,4,6,7-tetrahydro-5H- pyrazolo[4,3-c]pyridine-5- carboxamide |
| CPD- 1165 | 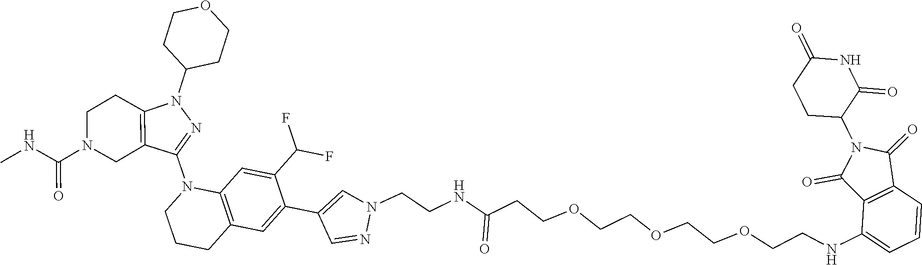 [SMILES: CNC(=O)N1CCc2c(c(N3CCCc4cc(-c5cnn(CCNC(=O)CCOCCOCCOCCNc6cccc7c6C(=O)N(C6CCC(=O)NC6=O)C7=O)c5)c(C(F)F)cc43)nn2C2CCOCC2)C1] | 3-(7-(difluoromethyl)-6-(1-(1-((2- (2,6-dioxopiperidin-3-yl)-1,3- dioxoisoindolin-4-yl)amino)-12- oxo-3,6,9-trioxa-13-azapentadecan- 15-yl)-1H-pyrazol-4-yl)-3,4- dihydroquinolin-1(2H)-yl)-N- methyl-1-(tetrahydro-2H-pyran-4- yl)-1,4,6,7-tetrahydro-5H- pyrazolo[4,3-c]pyridine-5- carboxamide |
| CPD- 1166 | 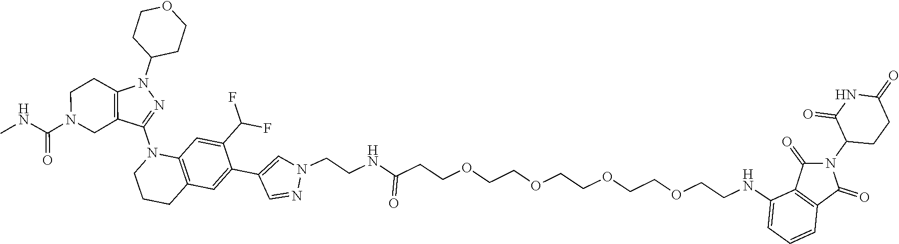 [SMILES: CNC(=O)N1CCc2c(c(N3CCCc4cc(-c5cnn(CCNC(=O)CCOCCOCCOCCOCCNc6cccc7c6C(=O)N(C6CCC(=O)NC6=O)C7=O)c5)c(C(F)F)cc43)nn2C2CCOCC2)C1] | 3-(7-(difluoromethyl)-6-(1-(1-((2- (2,6-dioxopiperidin-3-yl)-1,3- dioxoisoindolin-4-yl)amino)-15- oxo-3,6,9,12-tetraoxa-16- azaoctadecan-18-yl)-1H-pyrazol-4- yl)-3,4-dihydroquinolin-1(2H)-yl)- N-methyl-1-(tetrahydro-2H-pyran-4- yl)-1,4,6,7-tetrahydro-5H- pyrazolo[4,3-c]pyridine-5- carboxamide |
| CPD- 1167 | 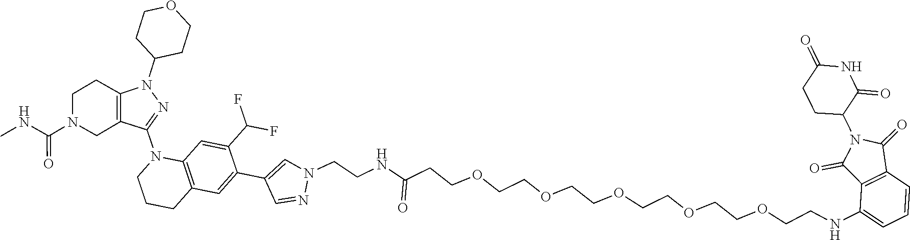 [SMILES: CNC(=O)N1CCc2c(c(N3CCCc4cc(-c5cnn(CCNC(=O)CCOCCOCCOCCOCCOCCNc6cccc7c6C(=O)N(C6CCC(=O)NC6=O)C7=O)c5)c(C(F)F)cc43)nn2C2CCOCC2)C1] | 3-(7-(difluoromethyl)-6-(1-(1-((2- (2,6-dioxopiperidin-3-yl)-1,3- dioxoisoindolin-4-yl)amino)-18- oxo-3,6,9,12,15-pentaoxa-19- azahenicosan-21-yl)-1H-pyrazol-4- yl)-3,4-dihydroquinolin-1(2H)-yl)- N-methyl-1-(tetrahydro-2H-pyran-4- yl)-1,4,6,7-tetrahydro-5H- pyrazolo[4,3-c]pyridine-5- carboxamide |
| CPD- 1168 | 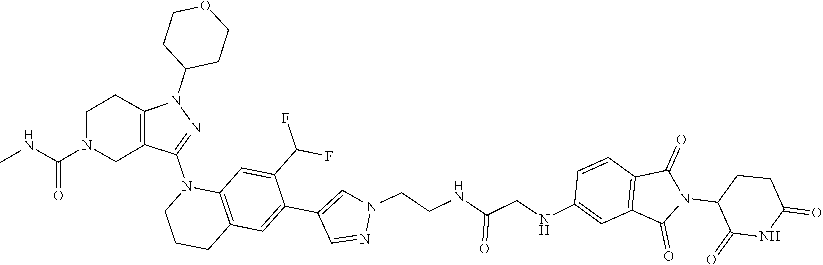 [SMILES: CNC(=O)N1CCc2c(c(N3CCCc4cc(-c5cnn(CCNC(=O)CNc6ccc7c(c6)C(=O)N(C6CCC(=O)NC6=O)C7=O)c5)c(C(F)F)cc43)nn2C2CCOCC2)C1] | 3-(7-(difluoromethyl)-6-(1-(2-(2- ((2-(2,6-dioxopiperidin-3-yl)-1,3- dioxoisoindolin-5- yl)amino)acetamido)ethyl)-1H- pyrazol-4-yl)-3,4-dihydroquinolin- 1(2H)-yl)-N-methyl-1-(tetrahydro- 2H-pyran-4-yl)-1,4,6,7-tetrahydro- 5H-pyrazolo[4,3-c]pyridine-5- carboxamide |
| CPD- 1169 | 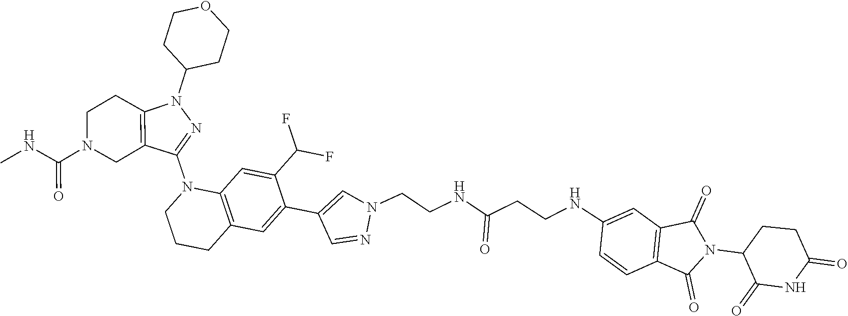 [SMILES: CNC(=O)N1CCc2c(c(N3CCCc4cc(-c5cnn(CCNC(=O)CCNc6ccc7c(c6)C(=O)N(C6CCC(=O)NC6=O)C7=O)c5)c(C(F)F)cc43)nn2C2CCOCC2)C1] | 3-(7-(difluoromethyl)-6-(1-(2-(3- ((2-(2,6-dioxopiperidin-3-yl)-1,3- dioxoisoindolin-5- yl)amino)propanamido)ethyl)-1H- pyrazol-4-yl)-3,4-dihydroquinolin- 1(2H)-yl)-N-methyl-1-(tetrahydro- 2H-pyran-4-yl)-1,4,6,7-tetrahydro- 5H-pyrazolo[4,3-c]pyridine-5- carboxamide |
| CPD- 1170 | 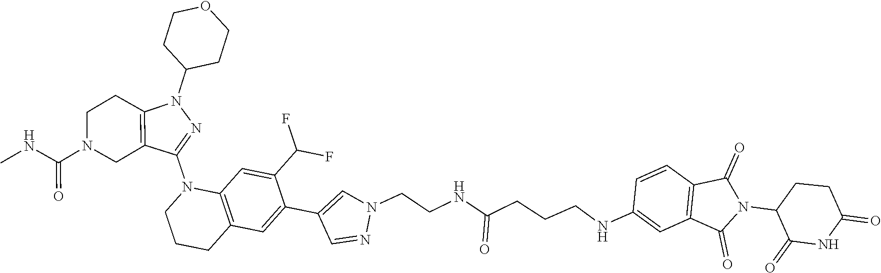 [SMILES: CNC(=O)N1CCc2c(c(N3CCCc4cc(-c5cnn(CCNC(=O)CCCNc6ccc7c(c6)C(=O)N(C6CCC(=O)NC6=O)C7=O)c5)c(C(F)F)cc43)nn2C2CCOCC2)C1] | 3-(7-(difluoromethyl)-6-(1-(2-(4- ((2-(2,6-dioxopiperidin-3-yl)-1,3- dioxoisoindolin-5- yl)amino)butanamido)ethyl)-1H- pyrazol-4-yl)-3,4-dihydroquinolin- 1(2H)-yl)-N-methyl-1-(tetrahydro- 2H-pyran-4-yl)-1,4,6,7-tetrahydro- 5H-pyrazolo[4,3-c]pyridine-5- carboxamide |
| CPD- 1171 | 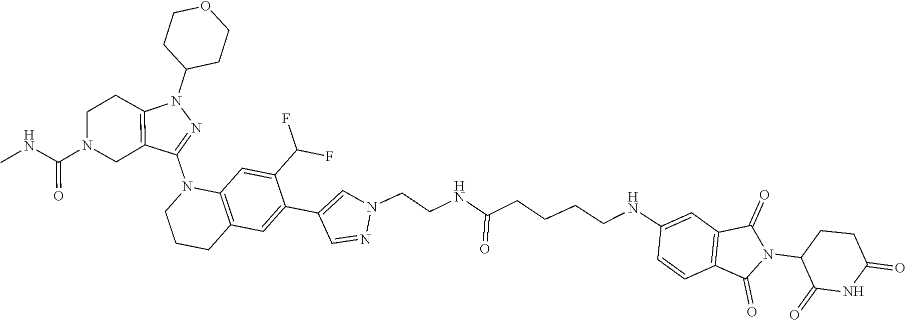 [SMILES: CNC(=O)N1CCc2c(c(N3CCCc4cc(-c5cnn(CCNC(=O)CCCCNc6ccc7c(c6)C(=O)N(C6CCC(=O)NC6=O)C7=O)c5)c(C(F)F)cc43)nn2C2CCOCC2)C1] | 3-(7-(difluoromethyl)-6-(1-(2-(5- ((2-(2,6-dioxopiperidin-3-yl)-1,3- dioxoisoindolin-5- yl)amino)pentanamido)ethyl)-1H- pyrazol-4-yl)-3,4-dihydroquinolin- 1(2H)-yl)-N-methyl-1-(tetrahydro- 2H-pyran-4-yl)-1,4,6,7-tetrahydro- 5H-pyrazolo[4,3-c]pyridine-5- carboxamide |
| CPD- 1172 | 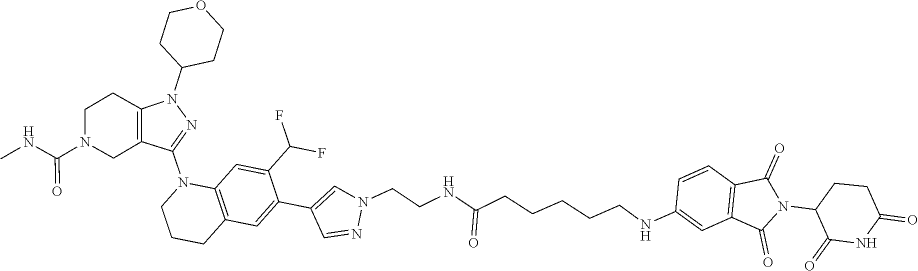 [SMILES: CNC(=O)N1CCc2c(c(N3CCCc4cc(-c5cnn(CCNC(=O)CCCCCNc6ccc7c(c6)C(=O)N(C6CCC(=O)NC6=O)C7=O)c5)c(C(F)F)cc43)nn2C2CCOCC2)C1] | 3-(7-(difluoromethyl)-6-(1-(2-(6- ((2-(2,6-dioxopiperidin-3-yl)-1,3- dioxoisoindolin-5- yl)amino)hexanamido)ethyl)-1H- pyrazol-4-yl)-3,4-dihydroquinolin- 1(2H)-yl)-N-methyl-1-(tetrahydro- 2H-pyran-4-yl)-1,4,6,7-tetrahydro- 5H-pyrazolo[4,3-c]pyridine-5- carboxamide |
| CPD- 1173 | 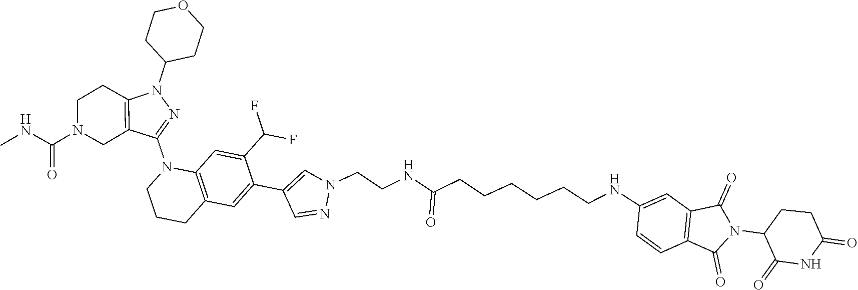 [SMILES: CNC(=O)N1CCc2c(c(N3CCCc4cc(-c5cnn(CCNC(=O)CCCCCCNc6ccc7c(c6)C(=O)N(C6CCC(=O)NC6=O)C7=O)c5)c(C(F)F)cc43)nn2C2CCOCC2)C1] | 3-(7-(difluoromethyl)-6-(1-(2-(7- ((2-(2,6-dioxopiperidin-3-yl)-1,3- dioxoisoindolin-5- yl)amino)heptanamido)ethyl)-1H- pyrazol-4-yl)-3,4-dihydroquinolin- 1(2H)-yl)-N-methyl-1-(tetrahydro- 2H-pyran-4-yl)-1,4,6,7-tetrahydro- 5H-pyrazolo[4,3-c]pyridine-5- carboxamide |
| CPD- 1174 | 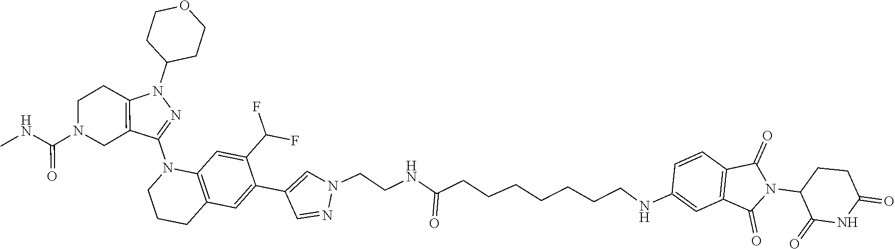 [SMILES: CNC(=O)N1CCc2c(c(N3CCCc4cc(-c5cnn(CCNC(=O)CCCCCCCNc6ccc7c(c6)C(=O)N(C6CCC(=O)NC6=O)C7=O)c5)c(C(F)F)cc43)nn2C2CCOCC2)C1] | 3-(7-(difluoromethyl)-6-(1-(2-(8- ((2-(2,6-dioxopiperidin-3-yl)-1,3- dioxoisoindolin-5- yl)amino)octanamido)ethyl)-1H- pyrazol-4-yl)-3,4-dihydroquinolin- 1(2H)-yl)-N-methyl-1-(tetrahydro- 2H-pyran-4-yl)-1,4,6,7-tetrahydro- 5H-pyrazolo[4,3-c]pyridine-5- carboxamide |
| CPD- 1175 | 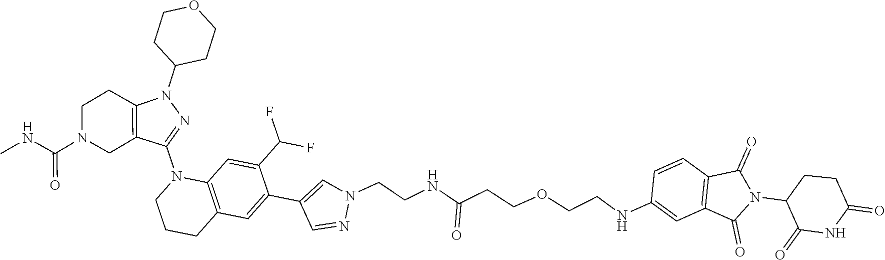 [SMILES: CNC(=O)N1CCc2c(c(N3CCCc4cc(-c5cnn(CCNC(=O)CCOCCNc6ccc7c(c6)C(=O)N(C6CCC(=O)NC6=O)C7=O)c5)c(C(F)F)cc43)nn2C2CCOCC2)C1] | 3-(7-(difluoromethyl)-6-(1-(2-(3-(2- ((2-(2,6-dioxopiperidin-3-yl)-1,3- dioxoisoindolin-5- yl)amino)ethoxy)propanamido) ethyl)-1H-pyrazol-4-yl)-3,4- dihydroquinolin-1(2H)-yl)-N- methyl-1-(tetrahydro-2H-pyran-4- yl)-1,4,6,7-tetrahydro-5H- pyrazolo[4,3-c]pyridine-5- carboxamide |
| CPD- 1176 | 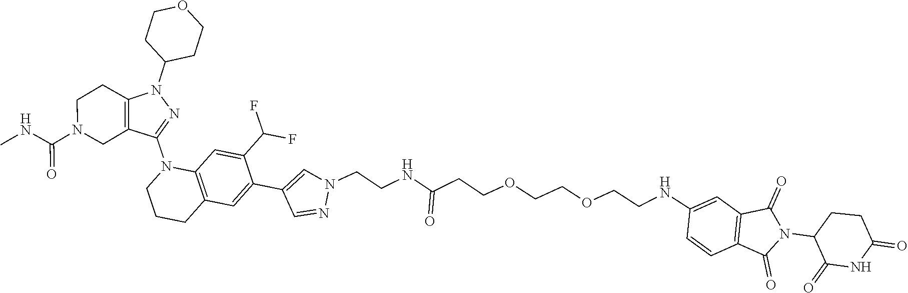 [SMILES: CNC(=O)N1CCc2c(c(N3CCCc4cc(-c5cnn(CCNC(=O)CCOCCOCCNc6ccc7c(c6)C(=O)N(C6CCC(=O)NC6=O)C7=O)c5)c(C(F)F)cc43)nn2C2CCOCC2)C1] | 3-(7-(difluoromethyl)-6-(1-(2-(3-(2- (2-((2-(2,6-dioxopiperidin-3-yl)-1,3- dioxoisoindolin-5-yl) amino)ethoxy)ethoxy)propanamido) ethyl)-1H-pyrazol-4-yl)-3,4- dihydroquinolin-1(2H)-yl)-N- methyl-1-(tetrahydro-2H-pyran-4- yl)-1,4,6,7-tetrahydro-5H- pyrazolo[4,3-c]pyridine-5- carboxamide |
| CPD- 1177 | 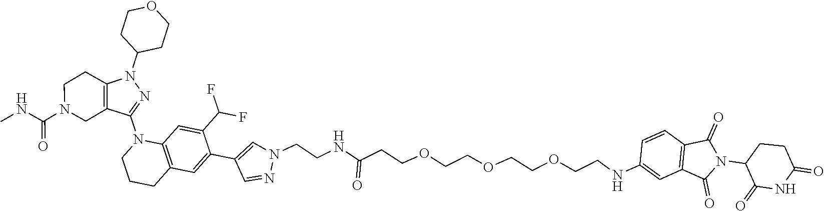 [SMILES: CNC(=O)N1CCc2c(c(N3CCCc4cc(-c5cnn(CCNC(=O)CCOCCOCCOCCNc6ccc7c(c6)C(=O)N(C6CCC(=O)NC6=O)C7=O)c5)c(C(F)F)cc43)nn2C2CCOCC2)C1] | 3-(7-(difluoromethyl)-6-(1-(1-((2- (2,6-dioxopiperidin-3-yl)-1,3- dioxoisoindolin-5-yl)amino)-12- oxo-3,6,9-trioxa-13-azapentadecan- 15-yl)-1H-pyrazol-4-yl)-3,4- dihydroquinolin-1(2H)-yl)-N- methyl-1-(tetrahydro-2H-pyran-4- yl)-1,4,6,7-tetrahydro-5H- pyrazolo[4,3-c]pyridine-5- carboxamide |
| CPD- 1178 | 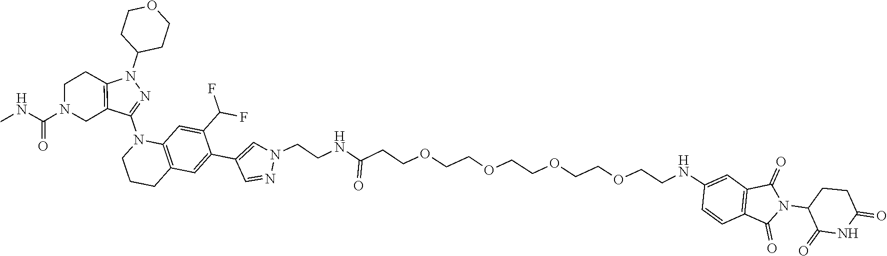 [SMILES: CNC(=O)N1CCc2c(c(N3CCCc4cc(-c5cnn(CCNC(=O)CCOCCOCCOCCOCCNc6ccc7c(c6)C(=O)N(C6CCC(=O)NC6=O)C7=O)c5)c(C(F)F)cc43)nn2C2CCOCC2)C1] | 3-(7-(difluoromethyl)-6-(1-(1-((2- (2,6-dioxopiperidin-3-yl)-1,3- dioxoisoindolin-5-yl)amino)-15- oxo-3,6,9,12-tetraoxa-16- azaoctadecan-18-yl)-1H-pyrazol-4- yl)-3,4-dihydroquinolin-1(2H)-yl)- N-methyl-1-(tetrahydro-2H-pyran-4- yl)-1,4,6,7-tetrahydro-5H- pyrazolo[4,3-c]pyridine-5- carboxamide |
| CPD- 1179 | 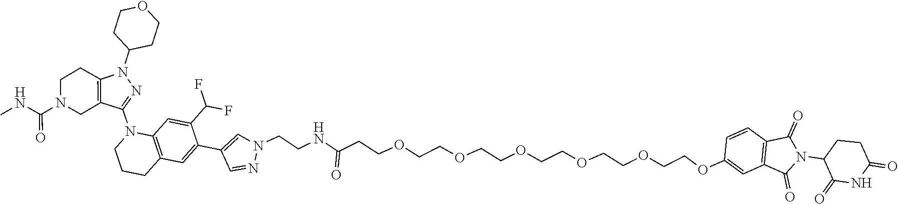 [SMILES: CNC(=O)N1CCc2c(c(N3CCCc4cc(-c5cnn(CCNC(=O)CCOCCOCCOCCOCCOCCOc6ccc7c(c6)C(=O)N(C6CCC(=O)NC6=O)C7=O)c5)c(C(F)F)cc43)nn2C2CCOCC2)C1] | 3-(7-(difluoromethyl)-6-(1-(1-((2- (2,6-dioxopiperidin-3-yl)-1,3- dioxoisoindolin-5-yl)amino)-18- oxo-3,6,9,12,15-pentaoxa-19- azahenicosan-21-yl)-1H-pyrazol-4- yl)-3,4-dihydroquinolin-1(2H)-yl)- N-methyl-1-(tetrahydro-2H-pyran-4- yl)-1,4,6,7-tetrahydro-5H- pyrazolo[4,3-c]pyridine-5- carboxamide |

As used herein, in case of discrepancy between the structure and chemical name provided for a particular compound, the structure shall control.

### Example 260. Bivalent Compounds Reduced P300 Protein Levels (FIG. 1)

LNCaP cells were treated with DMSO or indicated bivalent compounds at 5 nM for 6 hours. P300 protein levels were markedly reduced following treatment of some compounds as shown by immunoblotting assays.

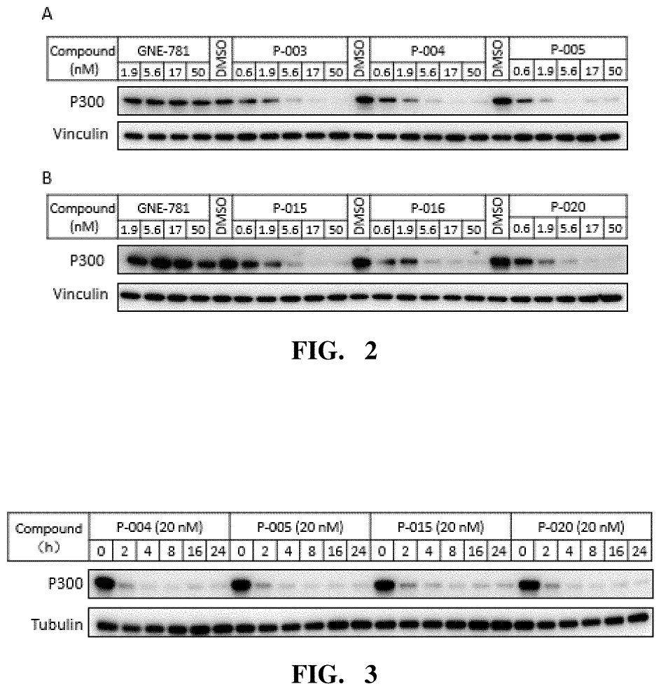

### Example 261. Bivalent Compounds Concentration Dependently Reduced P300 Protein Levels (FIG. 2)

LNCaP cells were treated with bivalent compounds at indicated concentrations for 16 hours. Data showed that P300 proteins levels were reduced in a concentration-dependent manner. The concentrations required to reduce P300 by 50% (DC₅₀) were below 5 nM for the selected compounds.

### Example 262. Bivalent Compounds Rapidly Reduced P300 Protein Levels (FIG. 3)

LNCaP cells were treated with selected bivalent compounds at 20 nM for indicated period of time. Data showed that P300 protein levels were significantly reduced as early as 2 hours following treatment.

### Example 263. Bivalent Compounds Suppressed Viability of LNCaP Prostate Cancer Cells (FIG. 4)

LNCaP cells were treated with GNE-781 or selected bivalent compounds for 3 days at indicated concentrations following a 3-fold serial dilution. Data showed that cell viability was significantly reduced in the presence of bivalent compounds in a concentration-dependent manner.

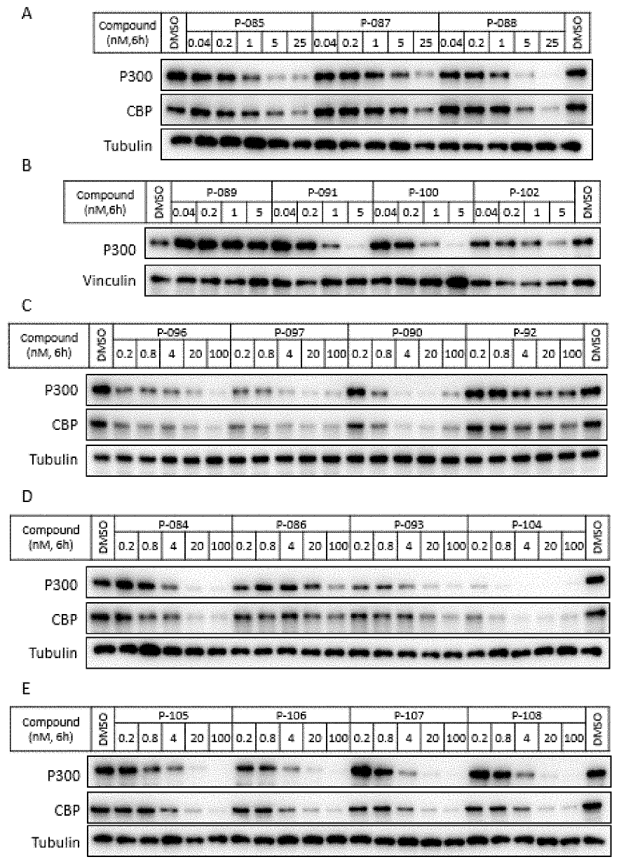

### Example 264. Bivalent Compounds Reduced P300/CBP Protein Levels (FIG. 5)

LNCaP cells were treated with DMSO or indicated bivalent compounds at 20 nM or 100 nM for 16 hours. P300/CBP protein levels were markedly reduced following treatment of some compounds as shown by immunoblotting assays.

### Example 265. Bivalent Compounds Concentration-Dependently Reduced P300/CBP Protein Levels (FIG. 6)

LNCaP cells were treated with compounds at indicated concentrations for 6 hours. Data showed that P300/CBP proteins levels were reduced in a concentration-dependent manner. The concentrations required to reduce P300/CBP by 50% (DC₅₀) were below 1 nM for the selected compounds.

### Example 266. Bivalent compound-mediated degradation of P300 is dependent on the interaction with cereblon (FIG. 7)

LNCaP or 22RV1 cells were treated with various concentrations of P-100 or P-100-negative. The latter lost binding to cereblon (CRBN) due to a chemical modification. Data showed that P-100 reduced P300 protein levels in a concentration-dependent manner while P-100-neg had no effects on P300 protein levels.

### Example 267. Bivalent Compound-Mediated Degradation of P300/CBP is Dependent on the Ubiquitin-Proteasome System (FIG. 8)

LNCaP cells were treated with a single dose of bivalent compounds, P-007, P-034 or P-100, or combination with pomalidomide, MG-132, Bortezomib, MLN4924. Data showed that bivalent compound-mediated degradation of P300/CBP is compromised by excessive CRBN ligand, pomalidomide, proteasome inhibitors, MG-132 or Bortezomib, or cullin E3 ligase inhibitor, MLN4924.

### Example 268. Bivalent Compounds Reduced P300/CBP Protein Levels in 22RV1 Subcutaneous Xenograft Tumors (FIG. 9)

Athymic nude mice bearing 22RV1 subcutaneous xenograft tumors at the right flank were intraperitoneally or orally treated with selected bivalent compounds at 40 mg/kg. Six hours after drug administration, animals were sacrificed, and xenograft tumors were collected for immunoblotting of P300 and CBP.

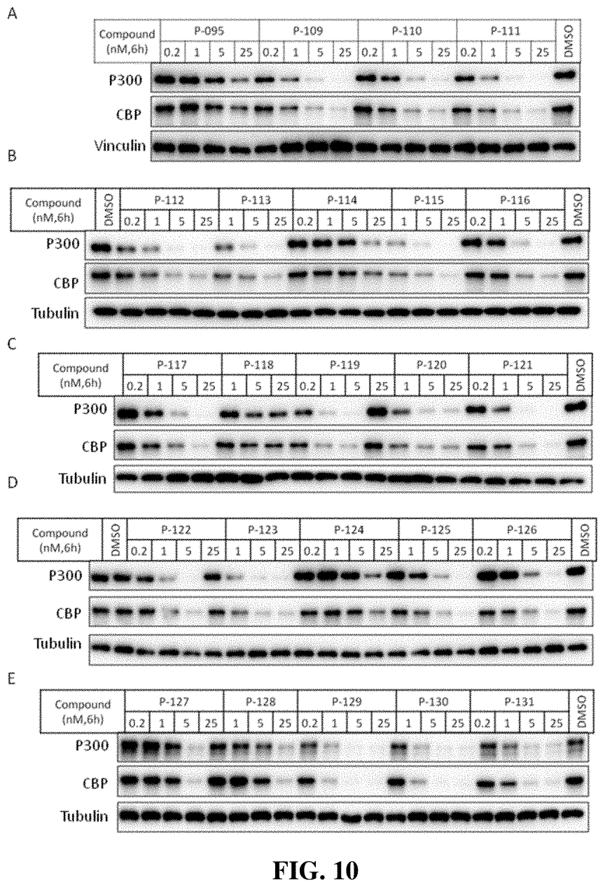

### Example 269. Bivalent Compounds Concentration-Dependently Reduced P300/CBP Protein Levels (FIG. 10)

LNCaP (FIG. 10A-B) or 22RV1 (FIG. 10C-E) cells were treated with bivalent compounds at indicated concentrations for 6 hours. Data showed that P300/CBP proteins levels were reduced in a concentration-dependent manner. The concentrations required to reduce P300/CBP by 50% (DC₅₀) were below 1 nM for the selected compounds.

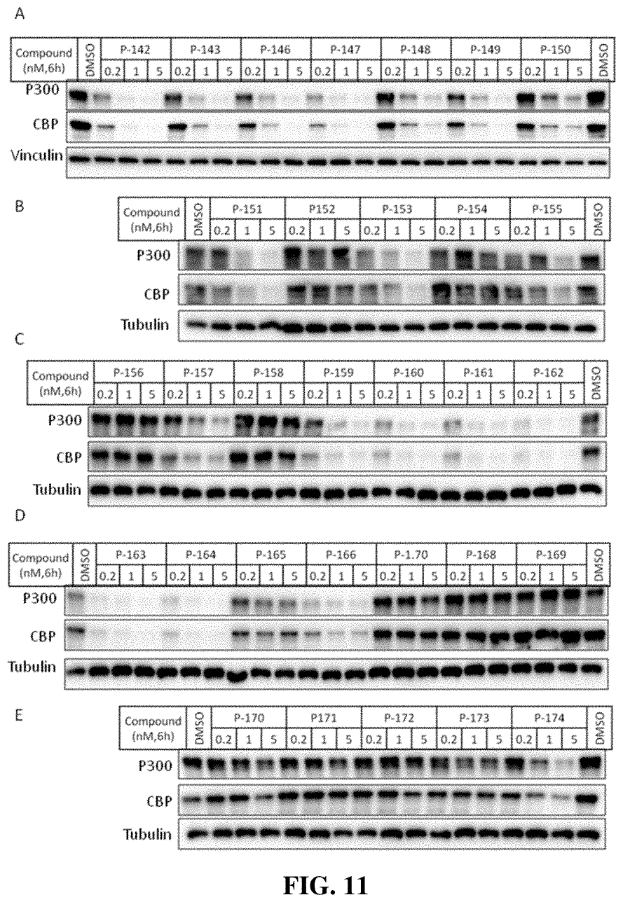

### Example 270. Bivalent Compounds Concentration-Dependently Reduced P300/CBP Protein Levels (FIG. 11)

LNCaP (FIG. 11B-E) or 22RV1 (FIG. 11A) cells were treated with bivalent compounds at indicated concentrations for 6 hours. Data showed that P300/CBP proteins levels were reduced in a concentration-dependent manner. The concentrations required to reduce P300/CBP by 50% (DC₅₀) were below 1 nM for the selected compounds.

### Example 271. Bivalent Compounds Reduced CBP Protein Levels in the Lung Tissues of ICR Mice (FIG. 12)

ICR mice were orally treated with 40 mg/kg bivalent compounds. Six hours after drug administration, animals were sacrificed, and the ling tissues were collected for immunoblotting of mouse CBP.

Materials and Methods:

General Chemistry Methods:

All chemicals and reagents were purchased from commercial suppliers and used without further purification. LCMS spectra for all compounds were acquired using a Shimadzu LC-MS 2020 system or a Waters UPLC-MS H class system. The Shimadzu LC-MS 2020 system comprising a pump (LC-20AD) with degasser (DGU-20A3), an autosampler (SIL-20AHT), a column oven (CTO-20A) (set at 40° C., unless otherwise indicated), a photo-diode array (PDA) (SPD-M20A) detector, an evaporative light-scattering (ELSD) (Alltech 3300ELSD) detector. Chromatography was performed on a Shimadzu SunFire C18 (5 μm 50*4.6 mm) with water containing 0.1% formic acid as solvent A and acetonitrile containing 0.1% formic acid as solvent B at a flow rate of 2.0 ml/min. Flow from the column was split to a MS spectrometer. The MS detector was configured with an electrospray ionization source. Nitrogen was used as the nebulizer gas. Data acquisition was performed with a Labsolution data system. The Waters UPLC-MS H class system comprising a pump (Quaternary Solvent Manager) with degasser, an autosampler (FTN), a column oven (set at 40° C., unless otherwise indicated), a photo-diode array PDA detector. Chromatography was performed on a AcQuity UPLC BEH C18 (1.7 μm 50*2.1 mm) with water containing 0.1% formic acid as solvent A and acetonitrile containing 0.1% formic acid as solvent B at a flow rate of 0.6 mL/min. Flow from the column was split to a MS spectrometer. The MS detector was configured with an electrospray ionization source. Nitrogen was used as the nebulizer gas. Data acquisition was performed with a MassLynx data system. Proton Nuclear Magnetic Resonance (¹H-NMR) spectra were recorded on a Bruker Avance 111400 spectrometer. Chemical shifts are expressed in parts per million (ppm) and reported as δ value (chemical shift δ). Coupling constants are reported in units of hertz (J value, Hz; Integration and splitting patterns: where s=singlet, d=double, t=triplet, q=quartet, brs=broad singlet, m=multiple). Preparative HPLC was performed on Agilent Prep 1260 series with UV detector set to 254 nm or 220 nm. Samples were injected onto a Phenomenex Luna 75×30 mm, 5 μm, C18 column at room temperature. The flow rate was 40 mL/min. A linear gradient was used with 10% (or 50%) of MeOH (A) in H₂O (with 0.1% TFA) (B) to 100% of MeOH (A). All compounds showed >90% purity using the LCMS methods described above.

Cell Culture

LNCaP (clone FGC), 22RV1 and other cells were cultured at 37° C. with 5% CO₂ in RPMI 1640 Medium supplemented with 10% fetal bovine serum. Cells were authenticated using the short tandem repeat (STR) assays. Mycoplasma test results were negative.

Antibodies and Reagents

Rabbit anti-P300 antibody (86377S), anti-CBP antibody (7389S) and anti-vinculin antibody (18799S) were purchased from Cell Signaling Technology. HRP-conjugated anti-tubulin antibody was produced in house. Media and other cell culture reagents were purchased from Thermo Fisher Scientific. The CellTiter-Glo Luminescent Assay kit was purchased from Promega.

Immunoblotting

Cultured cells or tissue chunks were washed with cold PBS once and lysed in cold RIPA buffer supplemented with protease inhibitors and phosphatase inhibitors (Beyotime Biotechnology). The solutions were then incubated at 4° C. for 30 minutes with gentle agitation to fully lyse cells. Cell lysates were centrifuged at 13,000 rpm for 10 minutes at 4° C. and pellets were discarded. Total protein concentrations in the lysates were determined by BCA assays (Beyotime Biotechnology). Cell lysates were mixed with Laemmli loading buffer to 1× and heated at 99° C. for 5 min. Proteins were resolved on SDS-PAGE and visualized by chemiluminescence. Images were taken by a ChemiDoc MP Imaging system (Bio-Rad). Protein bands were quantitated using the accompanied software provided by Bio-Rad.

Cell Viability Assays

Cells were seeded at a density of 5000 cells per well in 96-well assay plates and treated with test compounds following a 12-point 3-fold serial dilution. Three days later, cell viability was determined using the CellTiter-Glo assay kit according to the manufacturer's instructions. The dose-response curves were determined and IC₅₀ values were calculated using the GraphPad Prism software following a nonlinear regression (least squares fit) method.

The LNCaP prostate cancer cell viability inhibition results and the percentage of inhibition of p300 of selected bivalent compound compounds are set forth in Tables 2 and 3 below.

TABLE 2

| ID | IC₅₀ (nM) | Degradation (10 nM) |
|---|---|---|
| P-001 | 184.6 | B |
| P-002 | 42.1 | B |
| P-003 | 35.7 | B |
| P-004 | 7.1 | A |
| P-005 | 4 | A |
| P-006 | 3.2 | A |
| P-007 | 1.2 | A |
| P-008 | >10000 | C |
| P-009 | 114.5 | C |
| P-010 | 152.5 | C |
| P-011 | 163.3 | C |
| P-012 | 197.3 | D |
| P-013 | 105.1 | C |
| P-014 | >10000 | D |
| P-015 | 7.3 | A |
| P-016 | 3035 | D |
| P-017 | 99.2 | D |
| P-018 | 59.4 | C |
| P-019 | 14.7 | B |
| P-020 | 3.3 | A |
| P-021 | 278.1 | D |
| P-022 | 389.1 | D |
| P-023 | 357.4 | D |
| P-024 | 523.8 | D |
| P-025 | 1248 | D |
| P-026 | 3.2 | A |
| P-027 | 2.5 | A |
| P-028 | 1042 | D |
| P-029 | 578.3 | D |
| P-030 | 668.9 | D |
| P-031 | 566.2 | D |
| P-032 | 600.6 | D |
| P-033 | 4.8 | A |
| P-034 | 0.4 | A |
| P-035 | 0.7 | A |
| P-036 | 0.8 | A |
| P-037 | >10000 | D |
| P-038 | 384.9 | D |
| P-039 | 396.7 | D |
| P-040 | 96.4 | D |
| P-041 | 10.6 | C |
| P-042 | 39 | D |
| P-043 | 15.4 | C |
| P-044 | >10000 | D |
| P-045 | >10000 | D |
| P-046 | 9043 | D |
| P-047 | 2563 | D |
| P-048 | 470.7 | D |
| P-049 | 234.4 | D |
| P-050 | 111.3 | C |

A: Degradation > 80%; B: 50% < Degradation < 80%; C: 20% < Degradation < 50%; D: Degradation < 20%; ND: not determined.

TABLE 3

| ID | IC₅₀ (nM) | Degradation (10 nM) |
|---|---|---|
| P-051 | 27.5 | D |
| P-052 | 105.2 | D |
| P-053 | 76.5 | C |
| P-054 | 122.5 | D |
| P-055 | 161.3 | D |
| P-056 | 75.6 | D |
| P-057 | 76.6 | D |
| P-058 | 67.8 | D |
| P-059 | 60.6 | D |
| P-060 | 59.1 | D |
| P-061 | 81.3 | D |
| P-062 | 35 | C |
| P-063 | 28.9 | C |
| P-064 | 57.5 | D |
| P-065 | 74.5 | D |
| P-066 | 134.8 | D |
| P-067 | 32 | D |
| P-068 | 30.84 | D |
| P-069 | 36.1 | D |
| P-070 | 31.9 | D |
| P-071 | 106.6 | C |
| P-072 | 60.7 | D |
| P-073 | >10000 | D |
| P-074 | >10000 | D |
| P-075 | >10000 | D |
| P-076 | >10000 | D |
| P-077 | >10000 | D |
| P-078 | >10000 | D |
| P-079 | >10000 | D |
| P-080 | >10000 | D |
| P-081 | 468.7 | D |
| P-082 | >10000 | ND |
| P-083 | >10000 | ND |
| P-084 | 13.1 | B |
| P-085 | 3.8 | A |
| P-086 | 21.1 | B |
| P-087 | 12.6 | C |
| P-088 | 5.5 | A |
| P-089 | 78.5 | D |
| P-090 | 1 | A |
| P-091 | 2.1 | A |
| P-092 | 56.5 | C |
| P-093 | 25.2 | A |
| P-094 | 228.2 | ND |
| P-095 | 67.7 | C |
| P-096 | 26.6 | A |
| P-097 | 49 | A |
| P-098 | 1068 | ND |
| P-099 | >1000 | ND |
| P-100 | 0.9 | A |
| P-101 | >1000 | ND |
| P-102 | 14.9 | B |
| P-103 | 145.5 | ND |
| P-104 | 12.1 | A |
| P-105 | 44.5 | B |
| P-106 | 24.2 | B |
| P-107 | 20.4 | B |
| P-108 | 16.5 | B |
| P-109 | 12.9 | A |
| P-110 | 31.1 | A |
| P-111 | 7.6 | A |
| P-112 | 2.1 | A |
| P-113 | 14.8 | A |
| P-114 | 877.3 | C |
| P-115 | 7.1 | A |
| P-116 | 9.6 | A |
| P-117 | 41.2 | A |
| P-118 | 17.8 | C |
| P-119 | 1.7 | A |
| P-120 | 9.4 | A |
| P-121 | 28.8 | A |
| P-122 | 84.4 | A |
| P-123 | 20.3 | A |
| P-124 | >10000 | D |
| P-125 | 42.1 | A |
| P-126 | 94.0 | A |
| P-127 | 401.5 | C |
| P-128 | 243.7 | C |
| P-129 | 0.2 | A |
| P-130 | 0.5 | A |
| P-131 | 14.0 | A |
| P-132 | 16.8 | C |
| P-133 | 4.0 | A |
| P-134 | >500 | ND |
| P-135 | 0.7 | A |
| P-136 | >500 | ND |
| P-137 | >500 | ND |
| P-138 | >500 | ND |
| P-139 | >500 | ND |
| P-140 | >500 | ND |
| P-141 | 156 | B |
| P-142 | 0.3 | A |
| P-143 | 1.5 | A |
| P-144 | >1000 | ND |
| P-145 | >1000 | ND |
| P-146 | 2.0 | A |
| P-147 | 1.4 | A |
| P-148 | 73.3 | A |
| P-149 | 11.4 | A |
| P-150 | 191.2 | A |
| P-151 | 7.5 | A |
| P-152 | 169.1 | D |
| P-153 | 3.7 | A |
| P-154 | >1000 | C |
| P-155 | 22.3 | B |
| P-156 | >500 | D |
| P-157 | 3.2 | B |
| P-158 | 90.1 | D |
| P-159 | 0.2 | A |
| P-160 | 0.2 | A |
| P-161 | 0.1 | A |
| P-162 | 0.3 | A |
| P-163 | 0.9 | A |
| P-164 | 1.0 | A |
| P-165 | 123.4 | B |
| P-166 | 0.3 | A |
| P-167 | 92.0 | D |
| P-168 | 156.4 | D |
| P-169 | >500 | D |
| P-170 | 32.6 | C |
| P-171 | 252.9 | C |
| P-172 | 56.9 | C |
| P-173 | 3.4 | C |
| P-174 | 0.4 | A |

A: Degradation > 80%; B: 50% < Degradation < 80%; C: 20% < Degradation < 50%; D: Degradation < 20%; ND: not determined.

P-007 and P-034 potently inhibited cell viability of multiple cancel cell lines shown in Table 4 below.

TABLE 4

| Cell line | Disease | P-007 | P-034 | GNE-781 |
|---|---|---|---|---|
| HEL | Erythroleukemia | 16.1 | 37.8 | >10000 |
| MV4; 11 | Acute monocytic leukemia | 8.9 | >10000 | >10000 |
| Kasumi-1 | Acute monocytic leukemia | 87.8 | 1146.0 | 345.8 |
| NOMO-1 | Acute monocytic leukemia | — | 0.8 | >10000 |
| MOLM-13 | Acute monocytic leukemia | 7.7 | 6.9 | >10000 |
| HL-60 | Acute monocytic leukemia | 17.0 | 2.9 | >10000 |
| MEG-01 | Chronic myeloid leukemia | 7.9 | 15.3 | >10000 |
| MM.1S | Multiple Myeloma | 0.7 | 0.2 | 12.8 |
| MM.1R | Multiple Myeloma | 0.3 | 0.1 | 13.2 |
| NCI-H929 | Multiple Myeloma | 0.9 | 0.2 | 15.6 |
| L-363 | Multiple myeloma | 3.0 | 0.4 | >1000 |
| RPMI-8226 | Multiple myeloma | 3.7 | 9.4 | 10.3 |
| AMO-1 | Multiple Myeloma | 38.7 | 141.4 | 138.4 |
| WSU-DLCL2 | Diffuse large B-cell lymphoma | 0.4 | 0.4 | 14.0 |
| Karpas-422 | Diffuse large B-cell lymphoma | 6.2 | 0.9 | >10000 |
| Pfeiffer | Diffuse large B-cell lymphoma | 2.1 | 0.7 | 132.1 |
| SU-DHL-1 | Diffuse large B-cell lymphoma | 3.4 | 0.8 | >1000 |
| LNCaP clone FGC | Prostate carcinoma | 1.3 | 0.6 | 4167.0 |
| VCaP | Prostate carcinoma | 2.1 | 3.6 | 723.0 |
| 22Rv1 | Prostate carcinoma | 3.2 | 8.2 | 1684.0 |
| NCI-H520 | Non-small cell lung cancer | 2.8 | 2.0 | 5380.0 |
| NCI-H1703 | Non-small cell lung cancer | — | 2.9 | >10000 |
| LK-2 | Non-small cell lung cancer | 2.6 | 7.0 | 2711.0 |
| MCF-7 | breast carcinoma | 2.2 | 65.4 | 2392.0 |
| SK-BR-3 | breast carcinoma | 5.5 | 10.1 | >10000 |

The IC₅₀ value of each compound was determined as described in FIG. 4 and calculated using the GraphPad Prism 5.0 software.

Pharmacodynamic (PD) Studies

All animal experiments were performed under protocols approved by the Institutional Animal Care and Use Committee (IACUC) of Cullgen. Athymic nude mice (male, 5-weeks old) received 5 million 22RV1 cells subcutaneously inoculated at the right flank site. Twenty days following inoculation, tumors were approximately 500 mm³ in size. Tumor-bearing mice were treated intraperitoneally or via oral gavage with vehicle or bivalent compounds at indicated doses. 6 hours after drug administration, animals were sacrificed, tumors were resected. Small chunks of tumors were homogenized for immunoblotting of P300/CBP and other proteins as indicated. Alternatively, ICR mice (male, 5-weeks old) were treated via oral gavage with vehicle or bivalent compounds at indicated doses. 6 hours after drug administration, animals were sacrificed, lung tissues were resected. Small chunks of lung tissues were homogenized for immunoblotting of CBP and other proteins as indicated.

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

### REFERENCES

Bedford, D. C., Kasper, L. H., Fukuyama, T., and Brindle, P. K. (2010). Target gene context influences the transcriptional requirement for the KAT3 family of CBP and p300 histone acetyltransferases. Epigenetics 5, 9-15.
Blobel, G. A. (2000). CREB-binding protein and p300: molecular integrators of hematopoietic transcription. Blood 95, 745-755.
Bondeson, D. P., Mares, A., Smith, I. E., Ko, E., Campos, S., Miah, A. H., Mulholland, K. E., Routly, N., Buckley, D. L., Gustafson, J. L., et al. (2015). Catalytic in vivo protein knockdown by small-molecule PROTACs. Nat Chem Biol 11, 611-617.
Bronner, S. M., Murray, J., Romero, F. A., Lai, K. W., Tsui, V., Cyr, P., Beresini, M. H., de Leon Boenig, G., Chen, Z., Choo, E. F., et al. (2017). A Unique Approach to Design Potent and Selective Cyclic Adenosine Monophosphate Response Element Binding Protein, Binding Protein (CBP) Inhibitors. J Med Chem 60, 10151-10171.
Buckley, D. L., and Crews, C. M. (2014). Small-molecule control of intracellular protein levels through modulation of the ubiquitin proteasome system. Angew Chem Int Ed Engl 53, 2312-2330.
Buckley, D. L., Gustafson, J. L., Van Molle, I., Roth, A. G., Tae, H. S., Gareiss, P. C., Jorgensen, W. L., Ciulli, A., and Crews, C. M. (2012a). Small-molecule inhibitors of the interaction between the E3 ligase VHL and HIF1alpha. Angew Chem Int Ed Engl 51, 11463-11467.
Buckley, D. L., Raina, K., Darricarrere, N., Hines, J., Gustafson, J. L., Smith, I. E., Miah, A. H., Harling, J. D., and Crews, C. M. (2015). HaloPROTACS: Use of Small Molecule PROTACs to Induce Degradation of HaloTag Fusion Proteins. ACS Chem Biol 10, 1831-1837.
Buckley, D. L., Van Molle, I., Gareiss, P. C., Tae, H. S., Michel, J., Noblin, D. J., Jorgensen, W. L., Ciulli, A., and Crews, C. M. (2012b). Targeting the von Hippel-Lindau E3 ubiquitin ligase using small molecules to disrupt the VHL/HIF-1alpha interaction. J Am Chem Soc 134, 4465-4468.
Chakravarti, D., LaMorte, V. J., Nelson, M. C., Nakajima, T., Schulman, I. G., Juguilon, H., Montminy, M., and Evans, R. M. (1996). Role of CBP/P300 in nuclear receptor signalling. Nature 383, 99-103.
Chamberlain, P. P., Lopez-Girona, A., Miller, K., Carmel, G., Pagarigan, B., Chie-Leon, B., Rychak, E., Corral, L. G., Ren, Y. J., Wang, M., et al. (2014). Structure of the human Cereblon-DDB1-lenalidomide complex reveals basis for responsiveness to thalidomide analogs. Nat Struct Mol Biol 21, 803-809.
Dancy, B. M., and Cole, P. A. (2015). Protein lysine acetylation by p300/CBP. Chemical reviews 115, 2419-2452.
Davies, T. G., Wixted, W. E., Coyle, J. E., Griffiths-Jones, C., Hearn, K., McMenamin, R., Norton, D., Rich, S. J., Richardson, C., Saxty, G., et al. (2016). Monoacidic Inhibitors of the Kelch-like ECH-Associated Protein 1: Nuclear Factor Erythroid 2-Related Factor 2 (KEAP1:NRF2) Protein-Protein Interaction with High Cell Potency Identified by Fragment-Based Discovery. J Med Chem 59, 3991-4006.
Denny, R. A., Flick, A. C., Coe, J., Langille, J., Basak, A., Liu, S., Stock, I., Sahasrabudhe, P., Bonin, P., Hay, D. A., et al. (2017). Structure-Based Design of Highly Selective Inhibitors of the CREB Binding Protein Bromodomain. J Med Chem 60, 5349-5363.
Emami, K. H., Nguyen, C., Ma, H., Kim, D. H., Jeong, K. W., Eguchi, M., Moon, R. T., Teo, J. L., Kim, H. Y., Moon, S. H., et al. (2004). A small molecule inhibitor of beta-catenin/CREB-binding protein transcription [corrected]. Proc Natl Acad Sci USA 101, 12682-12687.
Faiola, F., Liu, X., Lo, S., Pan, S., Zhang, K., Lymar, E., Farina, A., and Martinez, E. (2005). Dual regulation of c-Myc by p300 via acetylation-dependent control of Myc protein turnover and coactivation of Myc-induced transcription. Molecular and cellular biology 25, 10220-10234.
Ferguson, J. H., De Los Santos, Z., Devi, S. N., Kaluz, S., Van Meir, E. G., Zingales, S. K., and Wang, B. (2017). Design and synthesis of benzopyran-based inhibitors of the hypoxia-inducible factor-1 pathway with improved water solubility. J Enzyme Inhib Med Chem 32, 992-1001.
Fischer, E. S., Bohm, K., Lydeard, J. R., Yang, H., Stadler, M. B., Cavadini, S., Nagel, J., Serluca, F., Acker, V., Lingaraju, G. M., et al. (2014). Structure of the DDB1-CRBN E3 ubiquitin ligase in complex with thalidomide. Nature 512, 49-53.
Galdeano, C., Gadd, M. S., Soares, P., Scaffidi, S., Van Molle, I., Birced, I., Hewitt, S., Dias, D. M., and Ciulli, A. (2014). Structure-guided design and optimization of small molecules targeting the protein-protein interaction between the von Hippel-Lindau (VHL) E3 ubiquitin ligase and the hypoxia inducible factor (HIF) alpha subunit with in vitro nanomolar affinities. J Med Chem 57, 8657-8663.
Giles, R. H., Peters, D. J., and Breuning, M. H. (1998). Conjunction dysfunction: CBP/p300 in human disease. Trends in genetics: TIG 14, 178-183.
Giotopoulos, G., Chan, W. I., Horton, S. J., Ruau, D., Gallipoli, P., Fowler, A., Crawley, C., Papaemmanuil, E., Campbell, P. J., Gottgens, B., et al. (2016). The epigenetic regulators CBP and p300 facilitate leukemogenesis and represent therapeutic targets in acute myeloid leukemia. Oncogene 35, 279-289.
Goodman, R. H., and Smolik, S. (2000). CBP/p300 in cell growth, transformation, and development. Genes & development 14, 1553-1577.
Hammitzsch, A., Tallant, C., Fedorov, O., O'Mahony, A., Brennan, P. E., Hay, D. A., Martinez, F. O., Al-Mossawi, M. H., de Wit, J., Vecellio, M., et al. (2015). CBP30, a selective CBP/p300 bromodomain inhibitor, suppresses human Th17 responses. Proc Natl Acad Sci USA 112, 10768-10773.
Hay, D. A., Fedorov, O., Martin, S., Singleton, D. C., Tallant, C., Wells, C., Picaud, S., Philpott, M., Monteiro, O. P., Rogers, C. M., et al. (2014). Discovery and optimization of small-molecule ligands for the CBP/p300 bromodomains. J Am Chem Soc 136, 9308-9319.
Hewings, D. S., Fedorov, O., Filippakopoulos, P., Martin, S., Picaud, S., Tumber, A., Wells, C., Olcina, M. M., Freeman, K., Gill, A., et al. (2013). Optimization of 3,5-dimethylisoxazole derivatives as potent bromodomain ligands. J Med Chem 56, 3217-3227.
Hewings, D. S., Wang, M., Philpott, M., Fedorov, O., Uttarkar, S., Filippakopoulos, P., Picaud, S., Vuppusetty, C., Marsden, B., Knapp, S., et al. (2011). 3,5-dimethylisoxazoles act as acetyl-lysine-mimetic bromodomain ligands. J Med Chem 54, 6761-6770.
Hugle, M., Lucas, X., Ostrovskyi, D., Regenass, P., Gerhardt, S., Einsle, O., Hau, M., Jung, M., Breit, B., Gunther, S., et al. (2017). Beyond the BET Family: Targeting CBP/p300 with 4-Acyl Pyrroles. Angew Chem Int Ed Engl 56, 12476-12480.
Ianari, A., Gallo, R., Palma, M., Alesse, E., and Gulino, A. (2004). Specific role for p300/CREB-binding protein-associated factor activity in E2F1 stabilization in response to DNA damage. The Journal of biological chemistry 279, 30830-30835.
Ianculescu, I., Wu, D. Y., Siegmund, K. D., and Stallcup, M. R. (2012). Selective roles for cAMP response element-binding protein binding protein and p300 protein as coregulators for androgen-regulated gene expression in advanced prostate cancer cells. The Journal of biological chemistry 287, 4000-4013.
Ito, T., Ando, H., Suzuki, T., Ogura, T., Hotta, K., Imamura, Y., Yamaguchi, Y., and Handa, H. (2010). Identification of a primary target of thalidomide teratogenicity. Science 327, 1345-1350.
Jin, L., Garcia, J., Chan, E., de la Cruz, C., Segal, E., Merchant, M., Kharbanda, S., Raisner, R., Haverty, P. M., Modrusan, Z., et al. (2017). Therapeutic Targeting of the CBP/p300 Bromodomain Blocks the Growth of Castration-Resistant Prostate Cancer. Cancer research 77, 5564-5575.
Kushal, S., Lao, B. B., Henchey, L. K., Dubey, R., Mesallati, H., Traaseth, N.J., Olenyuk, B. Z., and Arora, P. S. (2013). Protein domain mimetics as in vivo modulators of hypoxia-inducible factor signaling. Proc Natl Acad Sci USA 110, 15602-15607.
Lai, A. C., Toure, M., Hellerschmied, D., Salami, J., Jaime-Figueroa, S., Ko, E., Hines, J., and Crews, C. M. (2016). Modular PROTAC Design for the Degradation of Oncogenic BCR-ABL. Angew Chem Int Ed Engl 55, 807-810.
Lai, K. W., Romero, F. A., Tsui, V., Beresini, M. H., de Leon Boenig, G., Bronner, S. M., Chen, K., Chen, Z., Choo, E. F., Crawford, T. D., et al. (2018). Design and synthesis of a biaryl series as inhibitors for the bromodomains of CBP/P300. Bioorg Med Chem Lett 28, 15-23.
Lao, B. B., Grishagin, I., Mesallati, H., Brewer, T. F., Olenyuk, B. Z., and Arora, P. S. (2014). In vivo modulation of hypoxia-inducible signaling by topographical helix mimetics. Proc Natl Acad Sci USA 111, 7531-7536.
Lasko, L. M., Jakob, C. G., Edalji, R. P., Qiu, W., Montgomery, D., Digiammarino, E. L., Hansen, T. M., Risi, R. M., Frey, R., Manaves, V., et al. (2017). Discovery of a selective catalytic p300/CBP inhibitor that targets lineage-specific tumours. Nature 550, 128-132.
Liu, Y., Wang, L., Predina, J., Han, R., Beier, U. H., Wang, L. C., Kapoor, V., Bhatti, T. R., Akimova, T., Singhal, S., et al. (2013). Inhibition of p300 impairs Foxp3(+) T regulatory cell function and promotes antitumor immunity. Nature medicine 19, 1173-1177.
Lu, J., Qian, Y., Altieri, M., Dong, H., Wang, J., Raina, K., Hines, J., Winkler, J. D., Crew, A. P., Coleman, K., et al. (2015). Hijacking the E3 Ubiquitin Ligase Cereblon to Efficiently Target BRD4. Chemistry & biology 22, 755-763.
Martinez-Balbas, M. A., Bauer, U. M., Nielsen, S. J., Brehm, A., and Kouzarides, T. (2000). Regulation of E2F1 activity by acetylation. The EMBO journal 19, 662-671.
Nadiminty, N., Lou, W., Lee, S. O., Lin, X., Trump, D. L., and Gao, A. C. (2006). Stat3 activation of NF-{kappa}B p100 processing involves CBP/p300-mediated acetylation. Proceedings of the National Academy of Sciences of the United States of America 103, 7264-7269.
Ogiwara, H., Sasaki, M., Mitachi, T., Oike, T., Higuchi, S., Tominaga, Y., and Kohno, T. (2016). Targeting p300 Addiction in CBP-Deficient Cancers Causes Synthetic Lethality by Apoptotic Cell Death due to Abrogation of MYC Expression. Cancer discovery 6, 430-445. Ohoka, N., Okuhira, K., Ito, M., Nagai, K., Shibata, N., Hattori, T., Ujikawa, O.,
Shimokawa, K., Sano, O., Koyama, R., et al. (2017). In Vivo Knockdown of Pathogenic Proteins via Specific and Nongenetic Inhibitor of Apoptosis Protein (IAP)-dependent Protein Erasers (SNIPERs). J Biol Chem 292, 4556-4570.
Oike, T., Komachi, M., Ogiwara, H., Amornwichet, N., Saitoh, Y., Torikai, K., Kubo, N., Nakano, T., and Kohno, T. (2014). C646, a selective small molecule inhibitor of histone acetyltransferase p300, radiosensitizes lung cancer cells by enhancing mitotic catastrophe. Radiother Oncol 111, 222-227.
Okuhira, K., Ohoka, N., Sai, K., Nishimaki-Mogami, T., Itoh, Y., Ishikawa, M., Hashimoto, Y., and Naito, M. (2011). Specific degradation of CRABP-II via cIAP1-mediated ubiquitylation induced by hybrid molecules that crosslink cIAP1 and the target protein. FEBS Lett 585, 1147-1152.
Petrij, F., Giles, R. H., Dauwerse, H. G., Saris, J. J., Hennekam, R. C., Masuno, M., Tommerup, N., van Ommen, G. J., Goodman, R. H., Peters, D. J., et al. (1995). Rubinstein-Taybi syndrome caused by mutations in the transcriptional co-activator CBP. Nature 376, 348-351.
Picaud, S., Fedorov, O., Thanasopoulou, A., Leonards, K., Jones, K., Meier, J., Olzscha, H., Monteiro, O., Martin, S., Philpott, M., et al. (2015). Generation of a Selective Small Molecule Inhibitor of the CBP/p300 Bromodomain for Leukemia Therapy. Cancer Res 75, 5106-5119.
Popp, T. A., Tallant, C., Rogers, C., Fedorov, O., Brennan, P. E., Muller, S., Knapp, S., and Bracher, F. (2016). Development of Selective CBP/P300 Benzoxazepine Bromodomain Inhibitors. J Med Chem 59, 8889-8912.
Ramaswamy, K., Forbes, L., Minuesa, G., Gindin, T., Brown, F., Kharas, M. G., Krivtsov, A. V., Armstrong, S. A., Still, E., de Stanchina, E., et al. (2018). Peptidomimetic blockade of MYB in acute myeloid leukemia. Nat Commun 9, 110.
Rooney, T. P., Filippakopoulos, P., Fedorov, O., Picaud, S., Cortopassi, W. A., Hay, D. A., Martin, S., Tumber, A., Rogers, C. M., Philpott, M., et al. (2014). A series of potent CREBBP bromodomain ligands reveals an induced-fit pocket stabilized by a cation-pi interaction. Angew Chem Int Ed Engl 53, 6126-6130.
Rozman, M., Camos, M., Colomer, D., Villamor, N., Esteve, J., Costa, D., Carrio, A., Aymerich, M., Aguilar, J. L., Domingo, A., et al. (2004). Type I MOZ/CBP (MYST3/CREBBP) is the most common chimeric transcript in acute myeloid leukemia with t(8; 16)(p11; p13) translocation. Genes, chromosomes & cancer 40, 140-145.
Saha, R. N., and Pahan, K. (2006). HATs and HDACs in neurodegeneration: a tale of disconcerted acetylation homeostasis. Cell death and differentiation 13, 539-550.
Shi, Q., Yin, S., Kaluz, S., Ni, N., Devi, N. S., Mun, J., Wang, D., Damera, K., Chen, W., Burroughs, S., et al. (2012). Binding Model for the Interaction of Anticancer Arylsulfonamides with the p300 Transcription Cofactor. ACS Med Chem Lett 3, 620-625.
Shibata, N., Miyamoto, N., Nagai, K., Shimokawa, K., Sameshima, T., Ohoka, N., Hattori, T., Imaeda, Y., Nara, H., Cho, N., et al. (2017). Development of protein degradation inducers of oncogenic BCR-ABL protein by conjugation of ABL kinase inhibitors and IAP ligands. Cancer Sci 108, 1657-1666.
Sobulo, O. M., Borrow, J., Tomek, R., Reshmi, S., Harden, A., Schlegelberger, B., Housman, D., Doggett, N. A., Rowley, J. D., and Zeleznik-Le, N. J. (1997). MLL is fused to CBP, a histone acetyltransferase, in therapy-related acute myeloid leukemia with a t(11; 16)(q23; p13.3). Proceedings of the National Academy of Sciences of the United States of America 94, 8732-8737.
Sun, D., Li, Z., Rew, Y., Gribble, M., Bartberger, M. D., Beck, H. P., Canon, J., Chen, A., Chen, X., Chow, D., et al. (2014). Discovery of AMG 232, a potent, selective, and orally bioavailable MDM2-p53 inhibitor in clinical development. J Med Chem 57, 1454-1472.
Sun, Y., Kolligs, F. T., Hottiger, M. O., Mosavin, R., Fearon, E. R., and Nabel, G. J. (2000). Regulation of beta-catenin transformation by the p300 transcriptional coactivator. Proceedings of the National Academy of Sciences of the United States of America 97, 12613-12618.
Taylor, A. M., Cote, A., Hewitt, M. C., Pastor, R., Leblanc, Y., Nasveschuk, C. G., Romero, F. A., Crawford, T. D., Cantone, N., Jayaram, H., et al. (2016). Fragment-Based Discovery of a Selective and Cell-Active Benzodiazepinone CBP/EP300 Bromodomain Inhibitor (CPI-637). ACS Med Chem Lett 7, 531-536.
Unzue, A., Xu, M., Dong, J., Wiedmer, L., Spiliotopoulos, D., Caflisch, A., and Nevado, C. (2016). Fragment-Based Design of Selective Nanomolar Ligands of the CREBBP Bromodomain. J Med Chem 59, 1350-1356.
Uttarkar, S., Dukare, S., Bopp, B., Goblirsch, M., Jose, J., and Klempnauer, K. H. (2015). Naphthol AS-E Phosphate Inhibits the Activity of the Transcription Factor Myb by Blocking the Interaction with the KIX Domain of the Coactivator p300. Mol Cancer Ther 14, 1276-1285.
Valor, L. M., Viosca, J., Lopez-Atalaya, J. P., and Barco, A. (2013). Lysine acetyltransferases CBP and p300 as therapeutic targets in cognitive and neurodegenerative disorders. Curr Pharm Des 19, 5051-5064.
Vanden Berghe, W., De Bosscher, K., Boone, E., Plaisance, S., and Haegeman, G. (1999). The nuclear factor-kappaB engages CBP/p300 and histone acetyltransferase activity for transcriptional activation of the interleukin-6 gene promoter. The Journal of biological chemistry 274, 32091-32098.
Vervoorts, J., Luscher-Firzlaff, J. M., Rottmann, S., Lilischkis, R., Walsemann, G., Dohmann, K., Austen, M., and Luscher, B. (2003). Stimulation of c-MYC transcriptional activity and acetylation by recruitment of the cofactor CBP. EMBO reports 4, 484-490.
Wang, F., Marshall, C. B., and Ikura, M. (2013a). Transcriptional/epigenetic regulator CBP/p300 in tumorigenesis: structural and functional versatility in target recognition. Cellular and molecular life sciences: CMLS 70, 3989-4008.
Wang, N., Majmudar, C. Y., Pomerantz, W. C., Gagnon, J. K., Sadowsky, J. D., Meagher, J. L., Johnson, T. K., Stuckey, J. A., Brooks, C. L., 3rd, Wells, J. A., et al. (2013b). Ordering a dynamic protein via a small-molecule stabilizer. J Am Chem Soc 135, 3363-3366.
Wang, R., Cherukuri, P., and Luo, J. (2005). Activation of Stat3 sequence-specific DNA binding and transcription by p300/CREB-binding protein-mediated acetylation. The Journal of biological chemistry 280, 11528-11534.
Wang, R., He, Y., Robinson, V., Yang, Z., Hessler, P., Lasko, L. M., Lu, X., Bhathena, A., Lai, A., Uziel, T., et al. (2018). Targeting Lineage-specific MITF Pathway in Human Melanoma Cell Lines by A-485, the Selective Small-molecule Inhibitor of p300/CBP. Molecular cancer therapeutics 17, 2543-2550.
Wang, Y., Zhou, C., Gao, H., Li, C., Li, D., Liu, P., Huang, M., Shen, X., and Liu, L. (2017). Therapeutic effect of Cryptotanshinone on experimental rheumatoid arthritis through downregulating p300 mediated-STAT3 acetylation. Biochemical pharmacology 138, 119-129.
Wei, J., Yang, Y., Lu, M., Lei, Y., Xu, L., Jiang, Z., Xu, X., Guo, X., Zhang, X., Sun, H., et al. (2018). Recent Advances in the Discovery of HIF-1 alpha-p300/CBP Inhibitors as Anti-Cancer Agents. Mini reviews in medicinal chemistry 18, 296-309.
Winter, G. E., Buckley, D. L., Paulk, J., Roberts, J. M., Souza, A., Dhe-Paganon, S., and Bradner, J. E. (2015). Phthalimide conjugation as a strategy for in vivo target protein degradation. Science 348, 1376-1381.
Xie, T., Lim, S. M., Westover, K. D., Dodge, M. E., Ercan, D., Ficarro, S. B., Udayakumar, D., Gurbani, D., Tae, H. S., Riddle, S. M., et al. (2014). Pharmacological targeting of the pseudokinase Her3. Nat Chem Biol 10, 1006-1012.
Yang, H., Pinello, C. E., Luo, J., Li, D., Wang, Y., Zhao, L. Y., Jahn, S. C., Saldanha, S. A., Chase, P., Planck, J., et al. (2013). Small-molecule inhibitors of acetyltransferase p300 identified by high-throughput screening are potent anticancer agents. Mol Cancer Ther 12, 610-620.
Yin, S., Kaluz, S., Devi, N. S., Jabbar, A. A., de Noronha, R. G., Mun, J., Zhang, Z., Boreddy, P. R., Wang, W., Wang, Z., et al. (2012). Arylsulfonamide KCN1 inhibits in vivo glioma growth and interferes with HIF signaling by disrupting HIF-1alpha interaction with cofactors p300/CBP. Clin Cancer Res 18, 6623-6633.
Yusuke, H., Cu, N., Shin-Ya, Y., Michael, M., Kouichi, H., and Michael, K. (2016). Specific Direct Small Molecule p300/β-Catenin Antagonists Maintain Stem Cell Potency. Curr Mol Pharmacol 9, 272-279.
Zengerle, M., Chan, K. H., and Ciulli, A. (2015). Selective Small Molecule Induced Degradation of the BET Bromodomain Protein BRD4. ACS Chem Biol 10, 1770-1777.

1. A bivalent compound comprising a cyclic-AMP response element binding protein (CBP) and/or adenoviral E1A binding protein of 300 kDa (P300) ligand (CBP/P300 ligand) conjugated to a degradation tag, or a pharmaceutically acceptable salt or analog thereof.

2. The bivalent compound of claim 1, wherein the CBP/P300 ligand is capable of binding to a CBP/P300 protein comprising CBP/P300, a CBP/P300 mutant, CBP/P300 deletion, or a CBP/P300 fusion protein; and/or
the CBP/P300 ligand is a CBP/P300 inhibitor or a portion of CBP/P300 inhibitor.

3. (canceled)

4. The bivalent compound of claim 1, wherein the CBP/P300 ligand is selected from the group consisting of GNE-781, GNE-272, GNE-207, CPD 4d, CPD (S)-8, CPD (R)-2, CPD 6, CPD 19, XDM-CBP, I-CBP112, TPOP146, CPI-637, SGC-CBP30, CPD 11, CPD 41, CPD 30, CPD 5, CPD 29, CPD 27, C₆₄₆, A-485, naphthol-AS-E, MYBMIM, CCS1477, HBS1, OHM1, KCN1, ICG-001, YH249, YH250, and analogs thereof.

5. (canceled)

6. The bivalent compound of claim 1, wherein the degradation tag binds to an ubiquitin ligase, or is a hydrophobic group or a tag that leads to misfolding of the CBP/P300 protein.

7. The bivalent compound of claim 6, wherein the ubiquitin ligase is an E3 ligase.

8. The bivalent compound of claim 7, wherein the E3 ligase is selected from the group consisting of a cereblon E3 ligase, a VHL E3 ligase, an IAP ligase, a MDM2 ligase, a TRIM24 ligase, a TRIM21 ligase, a KEAP1 ligase, DCAF16 ligase, RNF4 ligase, RNF114 ligase, and AhR ligase.

9. (canceled)

10. The bivalent compound of claim 6, wherein the degradation tag is selected from the group consisting of pomalidomide, thalidomide, lenalidomide, VHL-1, adamantane, 1-((4,4,5,5,5-pentafluoropentyl)sulfinyl)nonane, nutlin-3a, RG7112, RG7338, AMG232, AA-115, bestatin, MV-1, LCL161, CPD36, GDC-0152, CRBN-1, CRBN-2, CRBN-3, CRBN-4, CRBN-5, CRBN-6, CRBN-7, CRBN-8, CRBN-9, CRBN-10, CRBN-11, and analogs thereof.

11. (canceled)

12. The bivalent compound of claim 1, wherein the CBP/P300 ligand is conjugated to the degradation tag via a linker moiety.

13. The bivalent compound of claim 12, wherein
(I)
(I-1) the CBP/P300 ligand comprises a moiety of FORMULA 1: 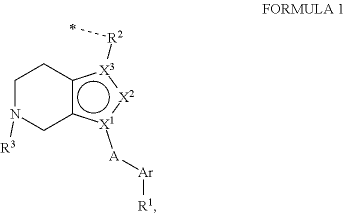
wherein
the linker moiety of the bivalent compound is attached to R²;
X¹ and X³ are independently selected from C and N, with the proviso that at least one of X¹ and X³ is C and at most only one of X¹ and X³ is N;
X² is selected from CR′, O, and NR′, wherein
R′ is selected from H, optionally substituted C₁-C₈ alkyl, and optionally substituted 3-10 membered carbocyclyl;
A is selected from null, CR⁴R⁵, CO, O, S, SO, SO₂, and NR⁴, wherein
R⁴ and R⁵ are independently selected from hydrogen, halogen, hydroxyl, cyano, nitro, optionally substituted C₁-C₈ alkyl, optionally substituted C₂-C₈ alkenyl, optionally substituted C₂-C₈ alkynyl, optionally substituted C₁-C₈ alkoxy, optionally substituted C₁-C₈alkoxyC₁-C₈alkyl, optionally substituted C₁-C₈ alkylamino, optionally substituted C₁-C₈alkylaminoC₁-C₈alkyl, optionally substituted 3-10 membered carbocyclyl, optionally substituted 3-8 membered cycloalkoxy, optionally substituted 3-10 membered carbocyclylamino, optionally substituted 4-10 membered heterocyclyl, optionally substituted aryl, and optionally substituted heteroaryl;
Ar and R⁴, Ar and R⁵, and/or R⁴ and R⁵ together with the atom to which they are connected form an optionally substituted 3-20 membered cycloalkyl or heterocyclyl ring;
Ar is selected from aryl, heteroaryl, bicyclic aryl, bicyclic heteroaryl, tricyclic aryl, tricyclic heteroaryl groups, each of which is substituted with R¹ and optionally substituted with one or more substituents independently selected from hydrogen, halogen, oxo, CN, NO₂, OR⁶, SR⁶, NR⁶R⁷, OCOR⁶, OCO₂R⁶, OCONR⁶R⁷, COR⁶, CO₂R⁶, CONR⁶R⁷, SOR⁶, SO₂R⁶, SO₂NR⁶R⁷, NR⁸CO₂R⁶, NR⁸COR⁶, NR⁸C(O)NR⁶R⁷, NR⁸SOR⁶, NR⁸SO₂R⁶, NR⁸SO₂NR⁶R⁷, optionally substituted C₁-C₈ alkyl, optionally substituted C₂-C₈ alkenyl, optionally substituted C₂-C₈ alkynyl, optionally substituted C₁-C₈ alkoxy, optionally substituted C₁-C₈alkoxyC₁-C₈alkyl, optionally substituted C₁-C₈alkylamino, optionally substituted C₁-C₈alkylaminoC₁-C₈alkyl, optionally substituted 3-10 membered carbocyclyl, optionally substituted 3-8 membered cycloalkoxy, optionally substituted 3-10 membered carbocyclylamino, optionally substituted 4-10 membered heterocyclyl, optionally substituted aryl, and optionally substituted heteroaryl,
wherein
R⁶, R⁷, and R⁸ are independently selected from hydrogen, optionally substituted C₁-C₈ alkyl, optionally substituted C₂-C₈ alkenyl, optionally substituted C₂-C₈ alkynyl, optionally substituted C₁-C₈alkoxyC₁-C₈alkyl, optionally substituted C₁-C₈alkylaminoC₁-C₈alkyl, optionally substituted 3-10 membered carbocyclyl, optionally substituted 4-10 membered heterocyclyl, optionally substituted aryl, and optionally substituted heteroaryl, or
R⁶ and R⁷, R⁶ and R⁸ together with the atom to which they are connected form a 4-20 membered heterocyclyl ring;
R¹ is selected from hydrogen, halogen, CN, NO₂, OR⁹, SR⁹, NR⁹R¹⁰, OCOR⁹, OCO₂R⁹, OCONR⁹R¹⁰, COR⁹, CO₂R⁹, CONR⁹R¹⁰, SOR⁹, SO₂R⁹, SO₂NR⁹R¹⁰, NR¹¹CO₂R⁹, NR¹¹COR⁹, NR¹¹C(O)NR⁹R¹⁰, NR¹¹SOR⁹, NR¹¹SO₂R⁹, NR¹¹SO₂NR⁹R¹⁰, optionally substituted C₁-C₈ alkyl, optionally substituted C₂-C₈ alkenyl, optionally substituted C₂-C₈ alkynyl, optionally substituted C₁-C₈ alkoxy, optionally substituted C₁-C₈alkoxyC₁-C₈alkyl, optionally substituted C₁-C₈alkylamino, optionally substituted C₁-C₈alkylaminoC₁-C₈alkyl, optionally substituted 3-10 membered carbocyclyl, optionally substituted 3-8 membered cycloalkoxy, optionally substituted 3-10 membered carbocyclylamino, optionally substituted 4-10 membered heterocyclyl, optionally substituted aryl, and optionally substituted heteroaryl, wherein
R⁹, R¹⁰, and R¹¹ are independently selected from hydrogen, optionally substituted C₁-C₈ alkyl, optionally substituted C₂-C₈ alkenyl, optionally substituted C₂-C₈ alkynyl, optionally substituted C₁-C₈alkoxyC₁-C₈alkyl, optionally substituted C₁-C₈alkylaminoC₁-C₈alkyl, optionally substituted 3-10 membered carbocyclyl, optionally substituted 4-10 membered heterocyclyl, optionally substituted aryl, and optionally substituted heteroaryl, or
R⁹ and R¹⁰, R⁹ and R¹¹ together with the atom to which they are connected form a 3-20 membered cycloalkyl or 4-20 membered heterocyclyl ring;
R² is connected to the “linker” moiety of the bivalent compound, and is selected from null, R″O, R″S, R″NR¹², R″OC(O), R″OC(O)O, R″OCON(R¹², R″C(O), R″C(O)O, R″CON(R¹², R″S(O), R″S(O)₂, R″SO₂NR¹², R″NR¹³C(O)O, R″NR¹³C(O), R″NR¹³C(O)NR¹², R″NR¹³S(O), R″NR¹³S(O)₂, R″NR¹³S(O)₂NR¹², optionally substituted C₁-C₈ alkylene, optionally substituted C₂-C₈ alkenylene, optionally substituted C₂-C₈ alkynylene, optionally substituted 3-10 membered carbocyclyl, optionally substituted 4-10 membered heterocyclyl, optionally substituted C₃-C₁₃ fused cycloalkyl, optionally substituted C₃-C₁₃ fused heterocyclyl, optionally substituted C₃-C₁₃ bridged cycloalkyl, optionally substituted C₃-C₁₃ bridged heterocyclyl, optionally substituted C₃-C₁₃ spiro cycloalkyl, optionally substituted C₃-C₁₃ spiro heterocyclyl, optionally substituted aryl, and optionally substituted heteroaryl, wherein
R″ is null, or a bivalent moiety selected from optionally substituted C₁-C₈ alkylene, optionally substituted C₂-C₈ alkenylene, optionally substituted C₂-C₈ alkynylene, optionally substituted C₁-C₈alkoxyC₁-C₈alkylene, optionally substituted C₁-C₈ haloalkylene, optionally substituted C₁-C₈ hydroxyalkylene, optionally substituted 3-10 membered carbocyclyl, optionally substituted 4-10 membered heterocyclyl, optionally substituted C₃-C₁₃ fused cycloalkyl, optionally substituted C₃-C₁₃ fused heterocyclyl, optionally substituted C₃-C₁₃ bridged cycloalkyl, optionally substituted C₃-C₁₃ bridged heterocyclyl, optionally substituted C₃-C₁₃ spiro cycloalkyl, optionally substituted C₃-C₁₃ spiro heterocyclyl, optionally substituted aryl, and optionally substituted heteroaryl;
R¹² and R¹³ are independently selected from optionally substituted C₁-C₈ alkyl, optionally substituted C₂-C₈ alkenyl, optionally substituted C₂-C₈ alkynyl, optionally substituted C₁-C₈alkoxyC₁-C₈alkyl, optionally substituted C₁-C₈alkylaminoC₁-C₈alkyl, optionally substituted 3-10 membered carbocyclyl, optionally substituted 4-10 membered heterocyclyl, optionally substituted aryl, and optionally substituted heteroaryl, or
R¹² and R¹³ together with the atom to which they are connected form a 3-20 membered cycloalkyl or 4-20 membered heterocyclyl ring; and
R³ is selected from hydrogen, COR¹⁴, CO₂R¹⁴, CONR¹⁴R¹⁵, SOR¹⁴, SO₂R¹⁴, SO₂NR¹⁴R¹⁵, optionally substituted C₁-C₆ alkyl, optionally substituted C₂-C₆ alkenyl, optionally substituted C₂-C₆ alkynyl, optionally substituted 3-6 membered cycloalkyl, optionally substituted 4-6 membered heterocyclyl, optionally substituted aryl, and optionally substituted heteroaryl, wherein
R¹⁴ and R¹⁵ are independently selected from hydrogen, optionally substituted C₁-C₆ alkyl, optionally substituted C₂-C₆ alkenyl, optionally substituted C₂-C₆ alkynyl, optionally substituted 3-6 membered cycloalkyl, optionally substituted 4-6 membered heterocyclyl, optionally substituted aryl, and optionally substituted heteroaryl, or
R¹⁴ and R¹⁵ together with the atom to which they are connected form a 4-20 membered heterocyclyl ring;
or, (I-2) the CBP/P300 ligand comprises a moiety of FORMULA 2: 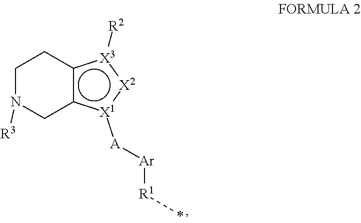
wherein
the linker moiety of the bivalent compound is attached to R¹; and
X¹ and X³ are independently selected from C and N, with the proviso that at least one of X¹ and
X³ is C and at most only one of X¹ and X³ is N;
X² is selected from CR′, O, and NR′, wherein
R′ is selected from H, optionally substituted C₁-C₈ alkyl, and optionally substituted 3-10 membered carbocyclyl;
A is selected from null, CR⁴R⁵, CO, O, S, SO, SO₂, and NR⁴, wherein
R⁴ and R⁵ are independently selected from hydrogen, halogen, hydroxyl, amino, cyano, nitro, optionally substituted C₁-C₈ alkyl, optionally substituted C₂-C₈ alkenyl, optionally substituted C₂-C₈ alkynyl, optionally substituted C₁-C₈ alkoxy, optionally substituted C₁-C₈alkoxyC₁-C₈alkyl, optionally substituted C₁-C₈ alkylamino, optionally substituted C₁-C₈alkylaminoC₁-C₈alkyl, optionally substituted 3-10 membered carbocyclyl, optionally substituted 3-8 membered cycloalkoxy, optionally substituted 3-10 membered carbocyclylamino, optionally substituted 4-10 membered heterocyclyl, optionally substituted aryl, and optionally substituted heteroaryl;
Ar and R⁴, Ar and R⁵, and/or R⁴ and R⁵ together with the atom to which they are connected form an optionally substituted 3-20 membered cycloalkyl or 4-20 membered heterocyclyl ring;
Ar is selected from aryl, heteroaryl, bicyclic aryl, bicyclic heteroaryl, tricyclic aryl, tricyclic heteroaryl groups, each of which is substituted with R¹ and optionally substituted with one or more substituents independently selected from hydrogen, halogen, oxo, CN, NO₂, OR⁶, SR⁶, NR⁶R⁷, OCOR⁶, OCO₂R⁶, OCONR⁶R⁷, COR⁶, CO₂R⁶, CONR⁶R⁷, SOR⁶, SO₂R⁶, SO₂NR⁶R⁷, NR⁸CO₂R⁶, NR⁸COR⁶, NR⁸C(O)NR⁶R⁷, NR⁸SOR⁶, NR⁸SO₂R⁶, NR⁸SO₂NR⁶R⁷, optionally substituted C₁-C₈ alkyl, optionally substituted C₂-C₈ alkenyl, optionally substituted C₂-C₈ alkynyl, optionally substituted C₁-C₈ alkoxy, optionally substituted C₁-C₈alkoxyC₁-C₈alkyl, optionally substituted C₁-C₈alkylamino, optionally substituted C₁-C₈alkylaminoC₁-C₈alkyl, optionally substituted 3-10 membered carbocyclyl, optionally substituted 3-8 membered cycloalkoxy, optionally substituted 3-10 membered carbocyclylamino, optionally substituted 4-10 membered heterocyclyl, optionally substituted aryl, and optionally substituted heteroaryl, wherein
R⁶, R⁷, and R⁸ are independently selected from hydrogen, optionally substituted C₁-C₈ alkyl, optionally substituted C₂-C₈ alkenyl, optionally substituted C₂-C₈ alkynyl, optionally substituted C₁-C₈alkoxyC₁-C₈alkyl, optionally substituted C₁-C₈alkylaminoC₁-C₈alkyl, optionally substituted 3-10 membered carbocyclyl, optionally substituted 4-10 membered heterocyclyl, optionally substituted aryl, and optionally substituted heteroaryl, or
R⁶ and R⁷, R⁶ and R⁸ together with the atom to which they are connected form a 4-20 membered heterocyclyl ring; and
R¹ is connected to the “linker” moiety of the bivalent compound, and R¹ is selected from null, R″O, R″S, R″NR⁹, R″OC(O), R″OC(O)O, R″OCONR⁹, R″C(O), R″C(O)O, R″CONR⁹, R″S(O), R″S(O)₂, R″SO₂NR⁹, R″NR¹⁰C(O)O, R″NR¹⁰C(O), R″NR¹⁰C(O)NR⁹, R″NR¹⁰S(O), R″NR¹⁰S(O)₂, R″NR¹⁰S(O)₂NR⁹, optionally substituted C₁-C₈ alkylene, optionally substituted C₂-C₈ alkenylene, optionally substituted C₂-C₈ alkynylene, optionally substituted 3-10 membered carbocyclyl, optionally substituted 4-8 membered heterocycly, optionally substituted aryl, and optionally substituted heteroaryl, wherein
R″ is null, or a bivalent moiety selected from optionally substituted C₁-C₈ alkylene, optionally substituted C₂-C₈ alkenylene, optionally substituted C₂-C₈ alkynylene, optionally substituted C₁-C₈alkoxyC₁-C₈alkylene, optionally substituted C₁-C₈ haloalkylene, optionally substituted C₁-C₈ hydroxyalkylene, optionally substituted 3-10 membered carbocyclyl, optionally substituted 4-10 membered heterocyclyl, optionally substituted C₃-C₁₃ fused cycloalkyl, optionally substituted C₃-C₁₃ fused heterocyclyl, optionally substituted C₃-C₁₃ bridged cycloalkyl, optionally substituted C₃-C₁₃ bridged heterocyclyl, optionally substituted C₃-C₁₃ spiro cycloalkyl, optionally substituted C₃-C₁₃ spiro heterocyclyl, optionally substituted aryl, and optionally substituted heteroaryl;
R⁹ and R¹⁰ are independently selected from optionally substituted C₁-C₈ alkyl, optionally substituted C₂-C₈ alkenyl, optionally substituted C₂-C₈ alkynyl, optionally substituted C₁-C₈alkoxyC₁-C₈alkyl, optionally substituted C₁-C₈alkylaminoC₁-C₈alkyl, optionally substituted 3-10 membered carbocyclyl, optionally substituted 4-10 membered heterocyclyl, optionally substituted aryl, and optionally substituted heteroaryl, or
R⁹ and R¹⁰ together with the atom to which they are connected form a 3-20 membered cycloalkyl or 4-20 membered heterocyclyl ring; and
R² is selected from hydrogen, halogen, CN, NO₂, OR¹¹, SR¹¹, NR¹¹R¹², OCOR¹¹, OCO₂R¹¹, OCONR¹¹R¹², COR¹¹, CO₂R¹¹, CONR¹¹R¹², SOR¹¹, SO₂R¹¹, SO₂NR¹¹R¹², NR¹³CO₂R¹¹, NR¹³COR¹¹, NR¹³C(O)NR¹¹R¹², NR¹³SOR¹¹, NR¹³SO₂R¹¹, NR¹³SO₂NR¹¹R¹², optionally substituted C₁-C₈ alkyl, optionally substituted C₂-C₈ alkenyl, optionally substituted C₂-C₈ alkynyl, optionally substituted C₁-C₈ alkoxy, optionally substituted C₁-C₈alkoxyC₁-C₈alkyl, optionally substituted C₁-C₈alkylamino, optionally substituted C₁-C₈alkylaminoC₁-C₈alkyl, optionally substituted 3-10 membered carbocyclyl, optionally substituted 3-8 membered cycloalkoxy, optionally substituted 3-10 membered carbocyclylamino, optionally substituted 4-10 membered heterocyclyl, optionally substituted aryl, and optionally substituted heteroaryl, wherein
R¹¹, R¹², and R¹³ are independently selected from hydrogen, optionally substituted C₁-C₈ alkyl, optionally substituted C₂-C₈ alkenyl, optionally substituted C₂-C₈ alkynyl, optionally substituted C₁-C₈alkoxyC₁-C₈alkyl, optionally substituted C₁-C₈alkylaminoC₁-C₈alkyl, optionally substituted 3-10 membered carbocyclyl, optionally substituted 4-10 membered heterocyclyl, optionally substituted aryl, and optionally substituted heteroaryl, or
R¹¹ and R¹², R¹¹ and R¹³ together with the atom to which they are connected form a 3-20 membered cycloalkyl or 4-20 membered heterocyclyl ring; and
R³ is selected from hydrogen, COR¹⁴, CO₂R¹⁴, CONR¹⁴R¹⁵, SOR¹⁴, SO₂R¹⁴, SO₂NR¹⁴R¹⁵, optionally substituted C₁-C₆ alkyl, optionally substituted C₂-C₆ alkenyl, optionally substituted C₂-C₆ alkynyl, optionally substituted 3-6 membered cycloalkyl, optionally substituted 4-6 membered heterocyclyl, wherein
R¹⁴ and R¹⁵ are independently selected from hydrogen, optionally substituted C₁-C₆ alkyl, optionally substituted C₂-C₆ alkenyl, optionally substituted C₂-C₆ alkynyl, optionally substituted 3-6 membered cycloalkyl, and optionally substituted 4-6 membered heterocyclyl, or
R¹⁴ and R¹⁵ together with the atom to which they are connected form a 4-6 membered heterocyclyl ring; and/or
(II)
(II-1) the degradation tag is a moiety selected from the group consisting of FORMULAE 5A, 5B, 5C, and 5D: 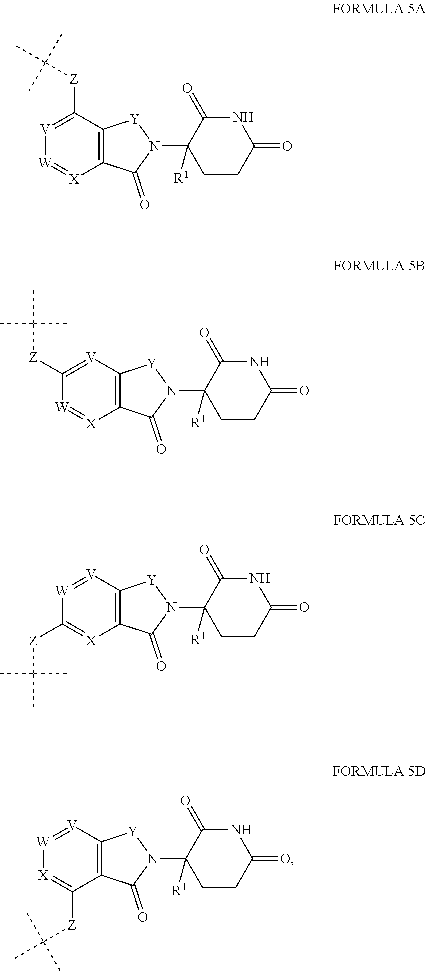
wherein
V, W, and X are independently selected from CR² and N;
Y is selected from CO, CR³R⁴, N═CR³, and N═N;
Z is selected from null, CO, CR⁵R⁶, NR⁵, O, optionally substituted C₁-C₁₀ alkylene, optionally substituted C₁-C₁₀ alkenylene, optionally substituted C₁-C₁₀ alkynylene, optionally substituted 3-10 membered carbocyclyl, optionally substituted 4-10 membered heterocyclyl, optionally substituted C₃-C₁₃ fused cycloalkyl, optionally substituted C₃-C₁₃ fused heterocyclyl, optionally substituted C₃-C₁₃ bridged cycloalkyl, optionally substituted C₃-C₁₃ bridged heterocyclyl, optionally substituted C₃-C₁₃ spiro cycloalkyl, optionally substituted C₃-C₁₃ spiro heterocyclyl, optionally substituted aryl, and optionally substituted heteroaryl; preferably, Z is selected from null, CH₂, CH═CH, C≡C, NH and O;
R¹, and R² are independently selected from hydrogen, halogen, cyano, nitro, optionally substituted C₁-C₆ alkyl, optionally substituted 3 to 6 membered carbocyclyl, and optionally substituted 4 to 6 membered heterocyclyl;
R³, and R⁴ are independently selected from hydrogen, halogen, cyano, nitro, optionally substituted C₁-C₆ alkyl, optionally substituted 3 to 6 membered carbocyclyl, and optionally substituted 4 to 6 membered heterocyclyl; or R³ and R⁴ together with the atom to which they are connected form a 3-6 membered carbocyclyl, or 4-6 membered heterocyclyl; and
R⁵ and R⁶ are independently selected from null, hydrogen, halogen, oxo, hydroxyl, amino, cyano, nitro, optionally substituted C₁-C₆ alkyl, optionally substituted 3 to 6 membered carbocyclyl, and optionally substituted 4 to 6 membered heterocyclyl; or R⁵ and R⁶ together with the atom to which they are connected form a 3-6 membered carbocyclyl, or 4-6 membered heterocyclyl;
or, (II-2) the degradation tag is a moiety selected from the group consisting of FORMULAE 5E, 5F, 5G, 5H, and 5I: 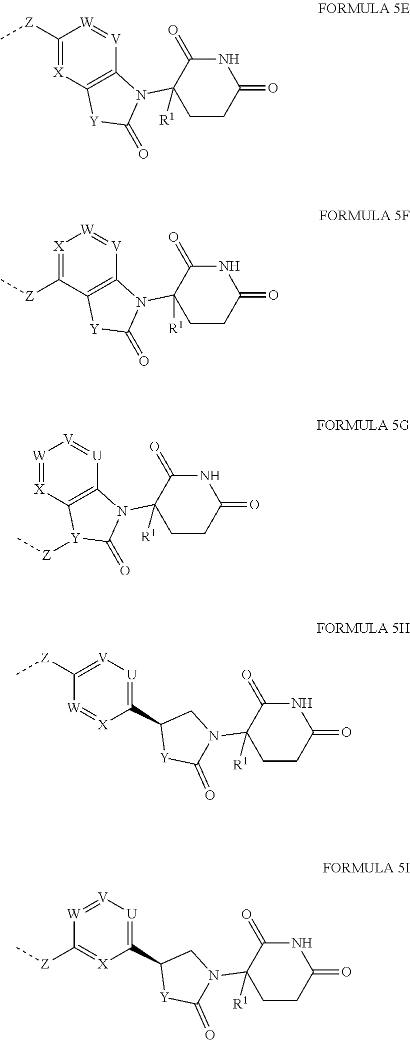
wherein
U, V, W, and X are independently selected from CR² and N;
Y is selected from CR³R⁴, NR³ and O; preferably, Y is selected from CH₂, NH, NCH₃ and O;
Z is selected from null, CO, CR⁵R⁶, NR⁵, O, optionally substituted C₁-C₁₀ alkylene, optionally substituted C₁-C₁₀ alkenylene, optionally substituted C₁-C₁₀ alkynylene, optionally substituted 3-10 membered carbocyclyl, optionally substituted 4-10 membered heterocyclyl, optionally substituted C₃-C₁₃ fused cycloalkyl, optionally substituted C₃-C₁₃ fused heterocyclyl, optionally substituted C₃-C₁₃ bridged cycloalkyl, optionally substituted C₃-C₁₃ bridged heterocyclyl, optionally substituted C₃-C₁₃ spiro cycloalkyl, optionally substituted C₃-C₁₃ spiro heterocyclyl, optionally substituted aryl, and optionally substituted heteroaryl; preferably, Z is selected from null, CH₂, CH═CH, C≡C, NH and O;
R¹, and R² are independently selected from hydrogen, halogen, cyano, nitro, optionally substituted C₁-C₆ alkyl, optionally substituted 3 to 6 membered carbocyclyl, and optionally substituted 4 to 6 membered heterocyclyl;
R³, and R⁴ are independently selected from hydrogen, halogen, cyano, nitro, optionally substituted C₁-C₆ alkyl, optionally substituted 3 to 6 membered carbocyclyl, and optionally substituted 4 to 6 membered heterocyclyl; or R³ and R⁴ together with the atom to which they are connected form a 3-6 membered carbocyclyl, or 4-6 membered heterocyclyl; and
R⁵ and R⁶ are independently selected from null, hydrogen, halogen, oxo, hydroxyl, amino, cyano, nitro, optionally substituted C₁-C₆ alkyl, optionally substituted 3 to 6 membered carbocyclyl, and optionally substituted 4 to 6 membered heterocyclyl; or R⁵ and R⁶ together with the atom to which they are connected form a 3-6 membered carbocyclyl, or 4-6 membered heterocyclyl;
or, (II-3) the degradation tag is a moiety selected from the group consisting of FORMULAE 5J, 5K, 5L, 5M, 5N, 5O, 5P, and 5Q 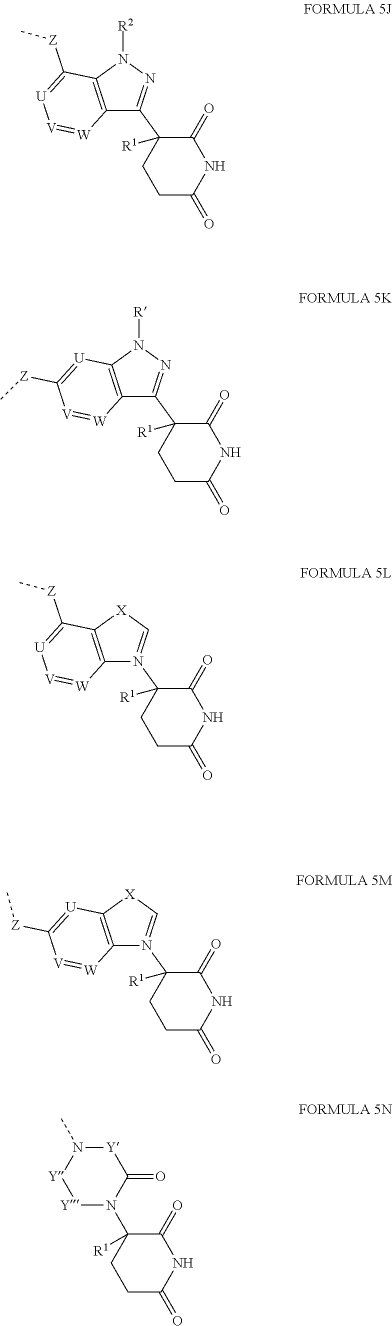 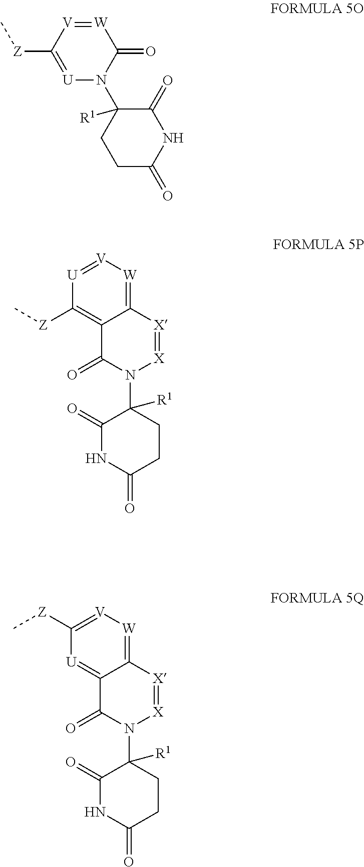
wherein,
X′ are independently selected from CR² and N;
Y′, Y″, and Y′″ are independently selected from CR³R⁴;
R′ is selected from hydrogen, optionally substituted C₁-C₆ alkyl, optionally substituted 3 to 6 membered carbocyclyl, and optionally substituted 4 to 6 membered heterocyclyl;
U, V, W, X, Z, R¹ and R² are defined as in FORMULAE 5E, 5F, 5G, 5H, or 5I;
or, (II-4) the degradation tag is a moiety of FORMULA 6A: 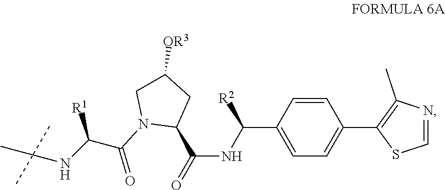
wherein
R¹ and R² are independently selected from hydrogen, hydroxyl, amino, cyano, nitro, optionally substituted C₁-C₈ alkyl, optionally substituted C₂-C₈ alkenyl, optionally substituted C₂-C₈ alkynyl; optionally substituted C₁-C₈alkoxyC₁-C₈alkyl, optionally substituted C₁-C₈ haloalkyl, optionally substituted C₁-C₈ hydroxyalkyl, optionally substituted C₁-C₈ aminoalkyl, optionally substituted C₁-C₈alkylaminoC₁-C₈alkyl, optionally substituted 3-10 membered carbocyclyl, and optionally substituted 4-10 membered heterocyclyl; and
R³ is selected from hydrogen, optionally substituted C(O)C₁-C₈ alkyl, optionally substituted C(O)C₁-C₈alkoxyC₁-C₈alkyl, optionally substituted C(O)C₁-C₈ haloalkyl, optionally substituted C(O)C₁-C₈ hydroxyalkyl, optionally substituted C(O)C₁-C₈ aminoalkyl, optionally substituted C(O)C₁-C₈alkylaminoC₁-C₈alkyl, optionally substituted C(O)(3-10 membered carbocyclyl), optionally substituted C(O)(4-10 membered heterocyclyl), optionally substituted C(O)C₂-C₈ alkenyl, optionally substituted C(O)C₂-C₈ alkynyl, optionally substituted C(O)OC₁-C₈alkoxyC₁-C₈alkyl, optionally substituted C(O)OC₁-C₈ haloalkyl, optionally substituted C(O)OC₁-C₈ hydroxyalkyl, optionally substituted C(O)OC₁-C₈ aminoalkyl, optionally substituted C(O)OC₁-C₈alkylaminoC₁-C₈alkyl, optionally substituted C(O)O(3-10 membered carbocyclyl), optionally substituted C(O)O(4-10 membered heterocyclyl), optionally substituted C(O)OC₂-C₈ alkenyl, optionally substituted C(O)OC₂-C₈ alkynyl, optionally substituted C(O)NC₁-C₈alkoxyC₁-C₈alkyl, optionally substituted C(O)NC₁-C₈ haloalkyl, optionally substituted C(O)NC₁-C₈ hydroxyalkyl, optionally substituted C(O)NC₁-C₈ aminoalkyl, optionally substituted C(O)NC₁-C₈alkylaminoC₁-C₈alkyl, optionally substituted C(O)N(3-10 membered carbocyclyl), optionally substituted C(O)N(4-10 membered heterocyclyl), optionally substituted C(O)NC₂-C₈ alkenyl, optionally substituted C(O)NC₂-C₈ alkynyl, optionally substituted P(O)(OH)₂, optionally substituted P(O)(OC₁-C₈ alkyl)₂, and optionally substituted P(O)(OC₁-C₈ aryl)₂;
or, (II-5) the degradation tag is a moiety of FORMULAE 6B, 6C, and 6D: 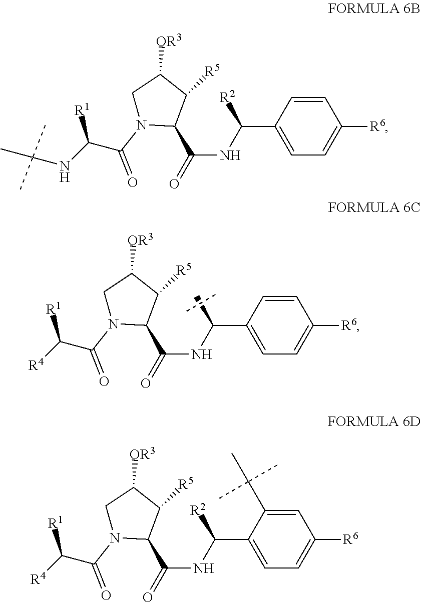
wherein
R¹ and R² are independently selected from hydrogen, hydroxyl, amino, cyano, nitro, optionally substituted C₁-C₈ alkyl, optionally substituted C₂-C₈ alkenyl, optionally substituted C₂-C₈ alkynyl; optionally substituted C₁-C₈alkoxyC₁-C₈alkyl, optionally substituted C₁-C₈ haloalkyl, optionally substituted C₁-C₈ hydroxyalkyl, optionally substituted C₁-C₈ aminoalkyl, optionally substituted C₁-C₈alkylaminoC₁-C₈alkyl, optionally substituted 3-10 membered carbocyclyl, and optionally substituted 4-10 membered heterocyclyl;
R³ is selected from hydrogen, optionally substituted C(O)C₁-C₈ alkyl, optionally substituted C(O)C₁-C₈alkoxyC₁-C₈alkyl, optionally substituted C(O)C₁-C₈ haloalkyl, optionally substituted C(O)C₁-C₈ hydroxyalkyl, optionally substituted C(O)C₁-C₈ aminoalkyl, optionally substituted C(O)C₁-C₈alkylaminoC₁-C₈alkyl, optionally substituted C(O)(3-10 membered carbocyclyl), optionally substituted C(O)(4-10 membered heterocyclyl), optionally substituted C(O)C₂-C₈ alkenyl, optionally substituted C(O)C₂-C₈ alkynyl, optionally substituted C(O)OC₁-C₈alkoxyC₁-C₈alkyl, optionally substituted C(O)OC₁-C₈ haloalkyl, optionally substituted C(O)OC₁-C₈ hydroxyalkyl, optionally substituted C(O)OC₁-C₈ aminoalkyl, optionally substituted C(O)OC₁-C₈alkylaminoC₁-C₈alkyl, optionally substituted C(O)O(3-10 membered carbocyclyl), optionally substituted C(O)O(4-10 membered heterocyclyl), optionally substituted C(O)OC₂-C₈ alkenyl, optionally substituted C(O)OC₂-C₈ alkynyl, optionally substituted C(O)NC₁-C₈alkoxyC₁-C₈alkyl, optionally substituted C(O)NC₁-C₈ haloalkyl, optionally substituted C(O)NC₁-C₈ hydroxyalkyl, optionally substituted C(O)NC₁-C₈ aminoalkyl, optionally substituted C(O)NC₁-C₈alkylaminoC₁-C₈alkyl, optionally substituted C(O)N(3-10 membered carbocyclyl), optionally substituted C(O)N(4-10 membered heterocyclyl), optionally substituted C(O)NC₂-C₈ alkenyl, optionally substituted C(O)NC₂-C₈ alkynyl, optionally substituted P(O)(OH)₂, optionally substituted P(O)(OC₁-C₈ alkyl)₂, and optionally substituted P(O)(OC₁-C₈ aryl)₂, and R⁴ is selected from NR⁷R⁸, 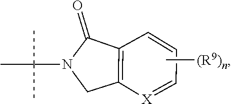
optionally substituted C₁-C₈alkoxy, optionally substituted 3-10 membered carbocyclyl, optionally substituted 4-10 membered heterocyclyl, optionally substituted aryl, and optionally substituted heteraryl, in which
R⁷ is selected from hydrogen, optionally substituted C₁-C₈alkyl, optionally substituted C₁-C₈cycloalkyl, optionally substituted C₁-C₈alkyl-CO, optionally substituted C₁-C₈cycloalkyl-CO, optionally substituted C₁-C₈cycloalkyl-C₁-C₈alkyl-CO, optionally substituted 4-10 membered heterocyclyl-CO, optionally substituted 4-10 membered heterocyclyl-C₁-C₈alkyl-CO, optionally substituted aryl-CO, optionally substituted aryl-C₁-C₈alkyl-CO, optionally substituted heteroaryl-CO, optionally substituted heteroaryl-C₁-C₈alkyl-CO, optionally substituted aryl, and optionally substituted heteroaryl;
R⁸ is selected from hydrogen, optionally substituted C₁-C₈alkyl, and optionally substituted C₁-C₈cycloalkyl;
R⁹, at each occurrence, is independently selected from hydrogen, halogen, cyano, optionally substituted C₁-C₈alkyl, optionally substituted C₁-C₈cycloalkyl, optionally substituted C₁-C₈heterocycloalkyl, optionally substituted C₁-C₈alkoxy, optionally substituted C₁-C₈cycloalkoxy, halo substituted C₁-C₈alkyl, halo substituted C₁-C₈cycloalkyl, halo substituted C₁-C₈alkoxl, halo substituted C₁-C₈cycloalkoxy, and halo substituted C₁-C₈heterocycloalkyl;
X is selected from CH and N; and
n is 0, 1, 2, 3, or 4;
R⁶ is selected from hydrogen, halogen, hydroxyl, amino, cyano, nitro, optionally substituted C₁-C₈alkyl, optionally substituted C₁-C₈cycloalkyl, optionally substituted C₁-C₈alkoxy, and optionally substituted C₁-C₈cycloalkoxy, optionally substituted C₁-C₈heterocycloalkyl, optionally substituted aryl, and optionally substituted heteroaryl, preferably, halogen, cyano, optionally substituted imidazole, optionally substituted pyrazole, optionally substituted oxadiazole, optionally substituted triazole, 4-methylthiazol-5-yl, or oxazol-5-yl group;
or, (II-6) the degradation tag is a moiety of FORMULA 7A: 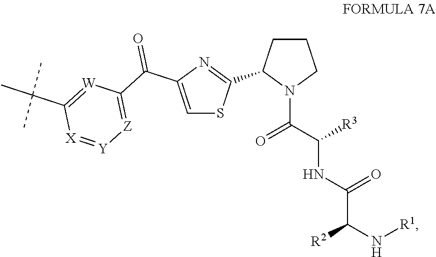
wherein
V, W, X, and Z are independently selected from CR⁴ and N; and
R¹, R², R³, and R⁴ are independently selected from hydrogen, halogen, cyano, nitro, optionally substituted C₁-C₈ alkyl, optionally substituted C₂-C₈ alkenyl, and optionally substituted C₂-C₈ alkynyl; optionally substituted C₁-C₈ alkoxyC₁-C₈ alkyl, optionally substituted C₁-C₈ halo alkyl, optionally substituted C₁-C₈ hydroxyalkyl, optionally substituted C₁-C₈alkoxy, optionally substituted C₁-C₈alkylamino, optionally substituted 3-10 membered carbocyclyl, and optionally substituted 4-10 membered heterocyclyl; and/or
(III)
(III-1) the linker moiety is of FORMULA 9: 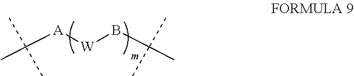
wherein
A, W and B, at each occurrence, are independently selected from null, or bivalent moiety selected from R′—R″, R′COR″, R′CO₂R″, R′C(O)N(R¹)R″, R′C(S)N(R¹)R″, R′OR″, R′SR″, R′SOR″, R′SO₂R″, R′SO₂N(R¹)R″, R′N(R¹)R″, R″N(R¹)COR″, R″N(R¹)CON(R²)R″, R′N(R¹)C(S)R″, optionally substituted C₁-C₈ alkylene, optionally substituted C₂-C₈ alkenylene, optionally substituted C₂-C₈ alkynylene, optionally substituted C₁-C₈alkoxyC₁-C₈alkylene, optionally substituted C₁-C₈ haloalkylene, optionally substituted C₁-C₈ hydroxyalkylene, optionally substituted C₃-C₁₃ fused cycloalkyl, optionally substituted C₃-C₁₃ fused heterocyclyl, optionally substituted C₃-C₁₃ bridged cycloalkyl, optionally substituted C₃-C₁₃ bridged heterocyclyl, optionally substituted C₃-C₁₃ spiro cycloalkyl, optionally substituted C₃-C₁₃ spiro heterocyclyl, optionally substituted 3-10 membered carbocyclyl, optionally substituted 4-10 membered heterocyclyl, optionally substituted aryl, and optionally substituted heteroaryl, wherein
R′ and R″ are independently selected from null, optionally substituted (C₁-C₈ alkylene)-R^{r} (preferably, CH₂-R^{r}), optionally substituted R^{r}—(C₁-C₈ alkylene), optionally substituted (C₁-C₈ alkylene)-R^{r}—(C₁-C₈ alkylene), or a moiety comprising of optionally substituted C₁-C₈ alkyl, optionally substituted C₂-C₈ alkenyl, optionally substituted C₂-C₈ alkynyl, optionally substituted C₁-C₈ hydroxyalkyl, optionally substituted C₁-C₈alkoxyC₁-C₈alkyl, optionally substituted C₁-C₈alkylaminoC₁-C₈alkyl, optionally substituted C₁-C₈ haloalkyl, optionally substituted C₁-C₈ alkylene, optionally substituted C₂-C₈ alkenylene, optionally substituted C₂-C₈ alkynylene, optionally substituted C₁-C₈ hydroxyalkylene, optionally substituted C₁-C₈alkoxyC₁-C₈alkylene, optionally substituted C₁-C₈alkylaminoC₁-C₈alkylene, optionally substituted C₁-C₈ haloalkylene, optionally substituted 3-10 membered carbocyclyl, optionally substituted 4-10 membered heterocyclyl, optionally substituted C₃-C₁₃ fused cycloalkyl, optionally substituted C₃-C₁₃ fused heterocyclyl, optionally substituted C₃-C₁₃ bridged cycloalkyl, optionally substituted C₃-C₁₃ bridged heterocyclyl, optionally substituted C₃-C₁₃ spiro cycloalkyl, optionally substituted C₃-C₁₃ spiro heterocyclyl, optionally substituted aryl, and optionally substituted heteroaryl;
R^{r} is selected from optionally substituted 3-10 membered carbocyclyl, optionally substituted 4-10 membered heterocyclyl, optionally substituted C₃-C₁₃ fused cycloalkyl, optionally substituted C₃-C₁₃ fused heterocyclyl, optionally substituted C₃-C₁₃ bridged cycloalkyl, optionally substituted C₃-C₁₃ bridged heterocyclyl, optionally substituted C₃-C₁₃ spiro cycloalkyl, optionally substituted C₃-C₁₃ spiro heterocyclyl, optionally substituted aryl, and optionally substituted heteroaryl;
R¹ and R² are independently selected from hydrogen, optionally substituted C₁-C₈ alkyl, optionally substituted C₂-C₈ alkenyl, optionally substituted C₂-C₈ alkynyl, optionally substituted C₁-C₈ alkoxyalkyl, optionally substituted C₁-C₈ haloalkyl, optionally substituted C₁-C₈ hydroxyalkyl, optionally substituted C₁-C₈alkylaminoC₁-C₈alkyl, optionally substituted 3-10 membered carbocyclyl, optionally substituted 4-10 membered heterocyclyl, optionally substituted aryl, and optionally substituted heteroaryl;
R′ and R″, R¹ and R², R′ and R¹, R′ and R², R″ and R¹, R″ and R² together with the atom to which they are connected form a 3-20 membered cycloalkyl or 4-20 membered heterocyclyl ring; and
m is 0 to 15;
or, (III-2) the linker moiety is of FORMULA 9A: 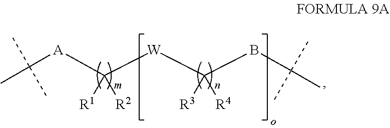
wherein
R¹, R², R³ and R⁴, at each occurrence, are independently selected from hydrogen, halogen, hydroxyl, amino, cyano, nitro, optionally substituted C₁-C₈ alkyl, optionally substituted C₂-C₈ alkenyl, optionally substituted C₂-C₈ alkynyl, optionally substituted C₁-C₈ alkoxy, optionally substituted C₁-C₈ alkoxyalkyl, optionally substituted C₁-C₈ haloalkyl, optionally substituted C₁-C₈ hydroxyalkyl, optionally substituted C₁-C₈ alkylamino, and optionally substituted C₁-C₈ alkylaminoC₁-C₈ alkyl, optionally substituted 3-10 membered carbocyclyl, optionally substituted 3-8 membered cycloalkoxy, optionally substituted 3-10 membered carbocyclylamino, optionally substituted 4-8 membered heterocyclyl, optionally substituted aryl, and optionally substituted heteroaryl, or
R¹ and R², R³ and R⁴ together with the atom to which they are connected form a 3-20 membered cycloalkyl or 4-20 membered heterocyclyl ring;
A, W and B, at each occurrence, are independently selected from null, or bivalent moiety selected from R′-R″, R′COR″, R′CO₂R″, R′C(O)N(R⁵)R″, R′C(S)N(R⁵)R″, R′OR″, R′SR″, R′SOR″, R′SO₂R″, R′SO₂N(R⁵)R″, R′N(R⁵)R″, R′N(R⁵)COR″, R′N(R⁵)CON(R⁶)R″, R′N(R⁵)C(S)R″, optionally substituted C₁-C₈ alkylene, optionally substituted C₂-C₈ alkenylene, optionally substituted C₂-C₈ alkynylene, optionally substituted C₁-C₈alkoxyC₁-C₈alkylene, optionally substituted C₁-C₈ haloalkylene, optionally substituted C₁-C₈ hydroxyalkylene, optionally substituted C₃-C₁₃ fused cycloalkyl, optionally substituted C₃-C₁₃ fused heterocyclyl, optionally substituted C₃-C₁₃ bridged cycloalkyl, optionally substituted C₃-C₁₃ bridged heterocyclyl, optionally substituted C₃-C₁₃ spiro cycloalkyl, optionally substituted C₃-C₁₃ spiro heterocyclyl, optionally substituted 3-10 membered carbocyclyl, optionally substituted 4-10 membered heterocyclyl, optionally substituted aryl, and optionally substituted heteroaryl,
wherein
R′ and R″ are independently selected from null, optionally substituted (C₁-C₈ alkyl)-R^{r} (preferably, CH₂—R^{r}), optionally substituted R^{r}—(C₁-C₈ alkylene), optionally substituted (C₁-C₈ alkylene)-R^{r}—(C₁-C₈ alkylene), or a moiety comprising of optionally substituted C₁-C₈ alkyl, optionally substituted C₂-C₈ alkenyl, optionally substituted C₂-C₈ alkynyl, optionally substituted C₁-C₈ hydroxyalkyl, optionally substituted C₁-C₈alkoxyC₁-C₈alkyl, optionally substituted C₁-C₈alkylaminoC₁-C₈alkyl, optionally substituted C₁-C₈ haloalkyl, optionally substituted C₁-C₈ alkylene, optionally substituted C₂-C₈ alkenylene, optionally substituted C₂-C₈ alkynylene, optionally substituted C₁-C₈ hydroxyalkylene, optionally substituted C₁-C₈alkoxyC₁-C₈alkylene, optionally substituted C₁-C₈alkylaminoC₁-C₈alkylene, optionally substituted C₁-C₈ haloalkylene, optionally substituted 3-10 membered carbocyclyl, optionally substituted 4-10 membered heterocyclyl, optionally substituted C₃-C₁₃ fused cycloalkyl, optionally substituted C₃-C₁₃ fused heterocyclyl, optionally substituted C₃-C₁₃ bridged cycloalkyl, optionally substituted C₃-C₁₃ bridged heterocyclyl, optionally substituted C₃-C₁₃ spiro cycloalkyl, optionally substituted C₃-C₁₃ spiro heterocyclyl, optionally substituted aryl, and optionally substituted heteroaryl;
R^{r} is selected from optionally substituted 3-10 membered carbocyclyl, optionally substituted 4-10 membered heterocyclyl, optionally substituted C₃-C₁₃ fused cycloalkyl, optionally substituted C₃-C₁₃ fused heterocyclyl, optionally substituted C₃-C₁₃ bridged cycloalkyl, optionally substituted C₃-C₁₃ bridged heterocyclyl, optionally substituted C₃-C₁₃ spiro cycloalkyl, optionally substituted C₃-C₁₃ spiro heterocyclyl, optionally substituted aryl, and optionally substituted heteroaryl;
R⁵ and R⁶ are independently selected from hydrogen, optionally substituted C₁-C₈ alkyl, optionally substituted C₂-C₈ alkenyl, optionally substituted C₂-C₈ alkynyl, optionally substituted C₁-C₈ alkoxyalkyl, optionally substituted C₁-C₈ haloalkyl, optionally substituted C₁-C₈ hydroxyalkyl, optionally substituted C₁-C₈alkylaminoC₁-C₈alkyl, optionally substituted 3-10 membered carbocyclyl, optionally substituted 4-10 membered heterocyclyl, optionally substituted aryl, and optionally substituted heteroaryl;
R′ and R″, R⁵ and R⁶, R′ and R⁵, R′ and R⁶, R″ and R⁵, R″ and R⁶ together with the atom to which they are connected form a 3-20 membered cycloalkyl or 4-20 membered heterocyclyl ring;
m is 0 to 15;
n, at each occurrence, is 0 to 15; and
o is 0 to 15;
or, (III-3) the linker moiety is of FORMULA 9B: 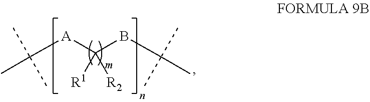
wherein
R¹ and R², at each occurrence, are independently selected from hydrogen, halogen, hydroxyl, amino, cyano, nitro, and optionally substituted C₁-C₈ alkyl, optionally substituted C₁-C₈ alkoxy, optionally substituted C₁-C₈ alkoxy C₁-C₈ alkyl, optionally substituted C₁-C₈ haloalkyl, optionally substituted C₁-C₈ hydroxyalkyl, optionally substituted C₁-C₈ alkylamino, C₁-C₈alkylaminoC₁-C₈alkyl, optionally substituted 3-10 membered carbocyclyl, optionally substituted 3-8 membered cycloalkoxy, optionally substituted 3-10 membered carbocyclylamino, optionally substituted 4-10 membered heterocyclyl, optionally substituted aryl, and optionally substituted heteroaryl, or
R¹ and R² together with the atom to which they are connected form a 3-20 membered cycloalkyl or 4-20 membered heterocyclyl ring;
A and B, at each occurrence, are independently selected from null, or bivalent moiety selected from R′-R″, R′COR″, R′CO₂R″, R′C(O)N(R³)R″, R′C(S)N(R³)R″, R′OR″, R′SR″, R′SOR″, R′SO₂R″, R′SO₂N(R³)R″, R′N(R³)R″, R′N(R³)COR″, R′N(R³)CON(R⁴)R″, R′N(R³)C(S)R″, optionally substituted C₁-C₈ alkylene, optionally substituted C₂-C₈ alkenylene, optionally substituted C₂-C₈ alkynylene, optionally substituted C₁-C₈alkoxyC₁-C₈alkylene, optionally substituted C₁-C₈ haloalkylene, optionally substituted C₁-C₈ hydroxyalkylene, optionally substituted C₃-C₁₃ fused cycloalkyl, optionally substituted C₃-C₁₃ fused heterocyclyl, optionally substituted C₃-C₁₃ bridged cycloalkyl, optionally substituted C₃-C₁₃ bridged heterocyclyl, optionally substituted C₃-C₁₃ spiro cycloalkyl, optionally substituted C₃-C₁₃ spiro heterocyclyl, optionally substituted 3-10 membered carbocyclyl, optionally substituted 4-10 membered heterocyclyl, optionally substituted aryl, and optionally substituted heteroaryl, wherein
R′ and R″ are independently selected from null, optionally substituted (C₁-C₈ alkylene)-R^{r} (preferably, CH₂-R^{r}), optionally substituted R^{r}—(C₁-C₈ alkylene), optionally substituted (C₁-C₈ alkylene)-R^{r}—(C₁-C₈ alkylene), or a moiety comprising of optionally substituted C₁-C₈ alkyl, optionally substituted C₂-C₈ alkenyl, optionally substituted C₂-C₈ alkynyl, optionally substituted C₁-C₈ hydroxyalkyl, optionally substituted C₁-C₈alkoxyC₁-C₈alkyl, optionally substituted C₁-C₈alkylaminoC₁-C₈alkyl, optionally substituted C₁-C₈ haloalkyl, optionally substituted C₁-C₈ alkylene, optionally substituted C₂-C₈ alkenylene, optionally substituted C₂-C₈ alkynylene, optionally substituted C₁-C₈ hydroxyalkylene, optionally substituted C₁-C₈alkoxyC₁-C₈alkylene, optionally substituted C₁-C₈alkylaminoC₁-C₈alkylene, optionally substituted C₁-C₈ haloalkylene, optionally substituted 3-10 membered carbocyclyl, optionally substituted 4-10 membered heterocyclyl, optionally substituted C₃-C₁₃ fused cycloalkyl, optionally substituted C₃-C₁₃ fused heterocyclyl, optionally substituted C₃-C₁₃ bridged cycloalkyl, optionally substituted C₃-C₁₃ bridged heterocyclyl, optionally substituted C₃-C₁₃ spiro cycloalkyl, optionally substituted C₃-C₁₃ spiro heterocyclyl, optionally substituted aryl, and optionally substituted heteroaryl;
R^{r} is selected from optionally substituted 3-10 membered carbocyclyl, optionally substituted 4-10 membered heterocyclyl, optionally substituted C₃-C₁₃ fused cycloalkyl, optionally substituted C₃-C₁₃ fused heterocyclyl, optionally substituted C₃-C₁₃ bridged cycloalkyl, optionally substituted C₃-C₁₃ bridged heterocyclyl, optionally substituted C₃-C₁₃ spiro cycloalkyl, optionally substituted C₃-C₁₃ spiro heterocyclyl, optionally substituted aryl, and optionally substituted heteroaryl;
R³ and R⁴ are independently selected from hydrogen, optionally substituted C₁-C₈ alkyl, optionally substituted C₂-C₈ alkenyl, optionally substituted C₂-C₈ alkynyl, optionally substituted C₁-C₈ alkoxyalkyl, optionally substituted C₁-C₈ haloalkyl, optionally substituted C₁-C₈ hydroxyalkyl, optionally substituted C₁-C₈alkylaminoC₁-C₈alkyl, optionally substituted 3-10 membered carbocyclyl, optionally substituted 4-10 membered heterocyclyl, optionally substituted aryl, and optionally substituted heteroaryl;
R′ and R″, R³ and R⁴, R′ and R³, R′ and R⁴, R″ and R³, R″ and R⁴ together with the atom to which they are connected form a 3-20 membered cycloalkyl or 4-20 membered heterocyclyl ring;
each m is 0 to 15; and
n is 0 to 15;
or, (III-4) the linker moiety is of FORMULA 9C: 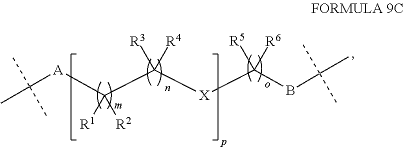
wherein
X is selected from O, NH, and NR⁷;
R¹, R², R³, R⁴, R⁵, and R⁶, at each occurrence, are independently selected from hydrogen, halogen, hydroxyl, amino, cyano, nitro, optionally substituted C₁-C₈ alkyl, optionally substituted C₂-C₈ alkenyl, optionally substituted C₂-C₈ alkynyl, optionally substituted C₁-C₈ alkoxy, optionally substituted C₁-C₈ alkoxy C₁-C₈ alkyl, optionally substituted C₁-C₈ haloalkyl, optionally substituted C₁-C₈ hydroxyalkyl, optionally substituted C₁-C₈ alkylamino, optionally substituted C₁-C₈ alkylaminoC₁-C₈ alkyl, optionally substituted 3-10 membered carbocyclyl, optionally substituted 3-8 membered cycloalkoxy, optionally substituted 4-10 membered heterocyclyl, optionally substituted aryl, and optionally substituted heteroaryl;
A and B are independently selected from null, or bivalent moiety selected from R′—R″, R′COR, R′CO₂R″, R′C(O)N(R⁸)R″, R′C(S)N(R⁸)R″, R′OR″, R′SR″, R′SOR″, R′SO₂R″, R′SO₂N(R⁸)R″, R′N(R⁸)R″, R′N(R⁸)COR″, R′N(R⁸)CON(R⁹)R″, R′N(R⁸)C(S)R″, optionally substituted C₁-C₈ alkylene, optionally substituted C₂-C₈ alkenylene, optionally substituted C₂-C₈ alkynylene, optionally substituted C₁-C₈alkoxyC₁-C₈alkylene, optionally substituted C₁-C₈ haloalkylene, optionally substituted C₁-C₈ hydroxyalkylene, optionally substituted C₃-C₁₃ fused cycloalkyl, optionally substituted C₃-C₁₃ fused heterocyclyl, optionally substituted C₃-C₁₃ bridged cycloalkyl, optionally substituted C₃-C₁₃ bridged heterocyclyl, optionally substituted C₃-C₁₃ spiro cycloalkyl, optionally substituted C₃-C₁₃ spiro heterocyclyl, optionally substituted 3-10 membered carbocyclyl, optionally substituted 4-10 membered heterocyclyl, optionally substituted aryl, and optionally substituted heteroaryl, wherein
R′ and R″ are independently selected from null, optionally substituted (C₁-C₈ alkylene)-R^{r} (preferably, CH₂-R^{r}), optionally substituted R^{r}—(C₁-C₈ alkylene), optionally substituted (C₁-C₈ alkylene)-R^{r}—(C₁-C₈ alkylene), or a moiety comprising of optionally substituted C₁-C₈ alkyl, optionally substituted C₂-C₈ alkenyl, optionally substituted C₂-C₈ alkynyl, optionally substituted C₁-C₈ hydroxyalkyl, optionally substituted C₁-C₈alkoxyC₁-C₈alkyl, optionally substituted C₁-C₈alkylaminoC₁-C₈alkyl, optionally substituted C₁-C₈ haloalkyl, optionally substituted C₁-C₈ alkylene, optionally substituted C₂-C₈ alkenylene, optionally substituted C₂-C₈ alkynylene, optionally substituted C₁-C₈ hydroxyalkylene, optionally substituted C₁-C₈alkoxyC₁-C₈alkylene, optionally substituted C₁-C₈alkylaminoC₁-C₈alkylene, optionally substituted C₁-C₈ haloalkylene, optionally substituted 3-10 membered carbocyclyl, optionally substituted 4-10 membered heterocyclyl, optionally substituted C₃-C₁₃ fused cycloalkyl, optionally substituted C₃-C₁₃ fused heterocyclyl, optionally substituted C₃-C₁₃ bridged cycloalkyl, optionally substituted C₃-C₁₃ bridged heterocyclyl, optionally substituted C₃-C₁₃ spiro cycloalkyl, optionally substituted C₃-C₁₃ spiro heterocyclyl, optionally substituted aryl, and optionally substituted heteroaryl;
R^{r} is selected from optionally substituted 3-10 membered carbocyclyl, optionally substituted 4-10 membered heterocyclyl, optionally substituted C₃-C₁₃ fused cycloalkyl, optionally substituted C₃-C₁₃ fused heterocyclyl, optionally substituted C₃-C₁₃ bridged cycloalkyl, optionally substituted C₃-C₁₃ bridged heterocyclyl, optionally substituted C₃-C₁₃ spiro cycloalkyl, optionally substituted C₃-C₁₃ spiro heterocyclyl, optionally substituted aryl, and optionally substituted heteroaryl;
R⁷, R⁸ and R⁹ are independently selected from hydrogen, optionally substituted C₁-C₈ alkyl, optionally substituted C₂-C₈ alkenyl, optionally substituted C₂-C₈ alkynyl, optionally substituted C₁-C₈ alkoxyalkyl, optionally substituted C₁-C₈ haloalkyl, optionally substituted C₁-C₈ hydroxyalkyl, optionally substituted C₁-C₈alkylaminoC₁-C₈alkyl, optionally substituted 3-10 membered carbocyclyl, optionally substituted 4-10 membered heterocyclyl, optionally substituted aryl, and optionally substituted heteroaryl;
R′ and R″, R⁸ and R⁹, R′ and R⁸, R′ and R⁹, R″ and R⁸, R″ and R⁹ together with the atom to which they are connected form a 3-20 membered cycloalkyl or 4-20 membered heterocyclyl ring;
m, at each occurrence, is 0 to 15;
n, at each occurrence, is 0 to 15;
o is 0 to 15; and
p is 0 to 15.

14. The bivalent compound of claim 13, wherein
in FORMULA 1, X¹ is C; X² and X³ are N; and the FORMULA 1 is FORMULA 1A: 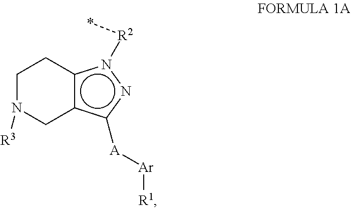
wherein
A, Ar, R¹, R² and R³ are the same as in FORMULA 1.

15. The bivalent compound of claim 13, wherein
in FORMULA 1, A-Ar—R¹ is a moiety of FORMULA A1: 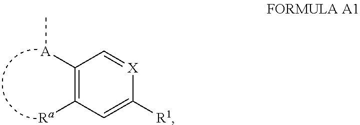
wherein
in FORMULA 1,
A and R¹ are the same as in FORMULA 1.
X is selected from CR′″ and N, wherein
R′″ is selected from hydrogen, halogen, hydroxyl, amino, cyano, nitro, optionally substituted C₁-C₆ alkyl, optionally substituted C₂-C₆ alkenyl, optionally substituted C₂-C₆ alkynyl, optionally substituted C₁-C₆ alkoxy, optionally substituted C₁-C₆ alkylamino, optionally substituted 3-6 membered cycloalkyl, optionally substituted 3-6 membered cycloalkoxy, optionally substituted 3-6 membered cycloalkylamino, optionally substituted 4-6 membered heterocyclyl, optionally substituted aryl, and optionally substituted heteroaryl; and
R^{a} optionally forms a ring with A, and is selected from null, hydrogen, halogen, R^{b}NR¹⁶, R^{b}OR¹⁶, R^{b}SR¹⁶, R^{b}NR¹⁶R¹⁷, R^{b}OCOR¹⁶, R^{b}OCO₂R¹⁶, R^{b}OCONR¹⁶R¹⁷, R^{b}COR¹⁶, R^{b}CO₂R¹⁶, R^{b}CONR¹⁶R¹⁷, R^{b}SOR¹⁶, R^{b}SO₂R¹⁶, R^{b}SO₂NR¹⁶R¹⁷, R^{b}NR¹⁸CO₂R¹⁶, R^{b}NR¹⁸COR¹⁶, R^{b}NR¹⁸C(O)NR¹⁶R¹⁷, R^{b}NR¹⁸SOR¹⁶, R^{b}NR¹⁸SO₂R¹⁶, R^{b}NR¹⁸SO₂NR¹⁶R¹⁷, optionally substituted C₁-C₈ alkyl, optionally substituted C₁-C₈ alkylene, optionally substituted C₂-C₈ alkenyl, optionally substituted C₂-C₈ alkenylene, optionally substituted C₂-C₈ alkynyl, optionally substituted C₂-C₈ alkynylene, optionally substituted 3-10 membered carbocyclyl, optionally substituted 4-10 membered heterocyclyl, optionally substituted aryl, and optionally substituted heteroaryl, wherein
R^{b} is null, or a bivalent or trivalent moiety selected from optionally substituted C₁-C₈ alkylene, optionally substituted C₂-C₈ alkenylene, optionally substituted C₂-C₈ alkynylene, optionally substituted C₁-C₈alkoxyC₁-C₈alkylene, optionally substituted C₁-C₈alkylaminoC₁-C₈alkylene, optionally substituted C₁-C₈ haloalkylene, optionally substituted C₁-C₈ hydroxyalkylene, optionally substituted 3-10 membered carbocyclyl, optionally substituted 4-10 membered heterocyclyl, optionally substituted aryl, and optionally substituted heteroaryl;
R¹⁶, R¹⁷, and R¹⁸ are independently selected from null, hydrogen, optionally substituted C₁-C₈ alkyl, optionally substituted C₂-C₈ alkenyl, optionally substituted C₂-C₈ alkynyl, optionally substituted C₁-C₈alkoxyC₁-C₈alkyl, optionally substituted C₁-C₈alkylaminoC₁-C₈alkyl, optionally substituted 3-10 membered carbocyclyl, optionally substituted 4-10 membered heterocyclyl, optionally substituted aryl, and optionally substituted heteroaryl, or R¹⁶ and R¹⁷, R¹⁶ and R¹⁸ together with the atom to which they are connected form a 3-20 membered cycloalkyl or heterocyclyl ring.

16. The bivalent compound of claim 13, wherein
in FORMULA 1,
A is null; or
A is NR⁴, wherein
R⁴ is selected from hydrogen, optionally substituted C₁-C₈ alkyl, optionally substituted C₂-C₈ alkenyl, optionally substituted C₂-C₈ alkynyl, optionally substituted C₁-C₈alkoxyC₁-C₈alkyl, optionally substituted C₁-C₈alkylaminoC₁-C₈alkyl, optionally substituted 3-10 membered carbocyclyl, optionally substituted 4-10 membered heterocyclyl, optionally substituted aryl, and optionally substituted heteroaryl.

17. The bivalent compound of claim 16, wherein
in FORMULA 1,
Ar—R¹ is a moiety of FORMULAE A2 or A3: 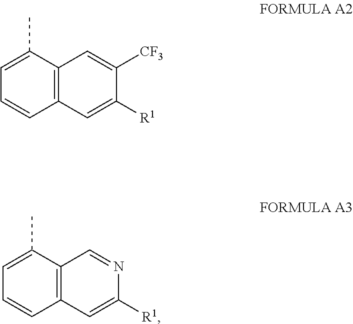
wherein
R¹ is the same as in FORMULA 1; or
A-Ar—R¹ is a moiety of FORMULAE A4, A5, or A6: 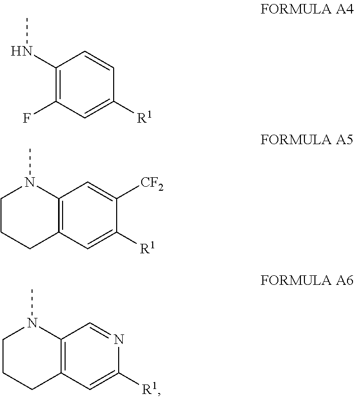
wherein
R¹ is the same as in FORMULA 1.

18-19. (canceled)

20. The bivalent compound of claim 13, wherein
in FORMULA 1,
R¹ is selected from optionally substituted 3-10 membered carbocyclyl, optionally substituted 4-10 membered heterocyclyl, optionally substituted aryl and optionally substituted heteroaryl;
and/or
R² is selected from optionally substituted C₁-C₈ alkylene, optionally substituted 3-10 membered carbocyclyl, optionally substituted 4-8 membered heterocycly, optionally substituted aryl, and optionally substituted heteroaryl; and/or
R³ is selected from COR¹⁴ and CONR¹⁴R¹⁵.

21. The bivalent compound of claim 13, wherein
in FORMULA 1,
R¹ is selected from optionally substituted aryl and optionally substituted heteroaryl.

22. The bivalent compound of claim 13, wherein
in FORMULA 1,
R¹ is selected from optionally substituted pyrazole and optionally substituted pyridinyl; and/or
R³ is selected from COMe and CONHMe.

23-26. (canceled)

27. The bivalent compound of claim 13, wherein
in FORMULA 2,
X¹ is C; X² and X³ are N; and the FORMULA 2 is FORMULA 2A: 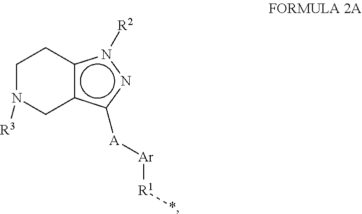
wherein
A, Ar, R¹, R² and R³ are the same as in FORMULA 2.

28. The bivalent compound of claim 13, wherein
in FORMULA 2,
A-Ar—R¹ is a moiety of FORMULA B1: 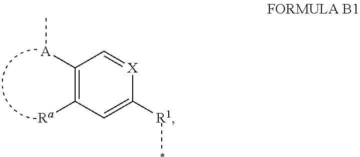
wherein
* indicates the connection to the linker moiety of the bivalent compound;
A and R¹ are the same as in FORMULA 2;
X is selected from CR′″ and N, wherein
R′″ is selected from hydrogen, halogen, hydroxyl, amino, cyano, nitro, optionally substituted C₁-C₆ alkyl, optionally substituted C₂-C₆ alkenyl, optionally substituted C₂-C₆ alkynyl, optionally substituted C₁-C₆ alkoxy, optionally substituted C₁-C₆ alkylamino, optionally substituted 3-6 membered cycloalkyl, optionally substituted 3-6 membered cycloalkyl, optionally substituted 3-6 membered cycloalkoxy, optionally substituted 3-6 membered cycloalkylamino, optionally substituted 4-6 membered heterocyclyl; and
R^{a} optionally forms a ring with A, and is selected from null, hydrogen, halogen, R^{b}NR¹⁶, R^{b}OR¹⁶, R^{b}SR¹⁶, R^{b}NR¹⁶R¹⁷, R^{b}OCOR¹⁶, R^{b}OCO₂R¹⁶, R^{b}OCONR¹⁶R¹⁷, R^{b}COR¹⁶, R^{b}CO₂R¹⁶, R^{b}CONR¹⁶R¹⁷, R^{b}SOR¹⁶, R^{b}SO₂R¹⁶, R^{b}SO₂NR¹⁶R¹⁷, R^{b}NR¹⁸CO₂R¹⁶, R^{b}NR¹⁸COR¹⁶, R^{b}NR¹⁸C(O)NR¹⁶R¹⁷, R^{b}NR¹⁸SOR¹⁶, R^{b}NR¹⁸SO₂R¹⁶, R^{b}NR¹⁸SO₂NR¹⁶R¹⁷, optionally substituted C₁-C₈ alkyl, optionally substituted C₁-C₈ alkylene, optionally substituted C₂-C₈ alkenyl, optionally substituted C₂-C₈ alkenylene, optionally substituted C₂-C₈ alkynyl, optionally substituted C₂-C₈ alkynylene, optionally substituted 3-10 membered carbocyclyl, optionally substituted 4-10 membered heterocyclyl, optionally substituted aryl, and optionally substituted heteroaryl, wherein
R^{b} is null, or a bivalent or trivalent moiety selected from optionally substituted C₁-C₈ alkylene, optionally substituted C₂-C₈ alkenylene, optionally substituted C₂-C₈ alkynylene, optionally substituted C₁-C₈alkoxyC₁-C₈alkylene, optionally substituted C₁-C₈alkylaminoC₁-C₈alkylene, optionally substituted C₁-C₈ haloalkylene, optionally substituted C₁-C₈ hydroxyalkylene, optionally substituted 3-10 membered carbocyclyl, optionally substituted 4-10 membered heterocyclyl, optionally substituted aryl, and optionally substituted heteroaryl;
R¹⁶, R¹⁷, and R¹⁸ are independently selected from null, a bond, hydrogen, optionally substituted C₁-C₈ alkyl, optionally substituted C₂-C₈ alkenyl, optionally substituted C₂-C₈ alkynyl, optionally substituted C₁-C₈alkoxyC₁-C₈alkyl, optionally substituted C₁-C₈alkylaminoC₁-C₈alkyl, optionally substituted 3-10 membered carbocyclyl, optionally substituted 4-10 membered heterocyclyl, optionally substituted aryl, and optionally substituted heteroaryl, or
R¹⁶ and R¹⁷, R¹⁶ and R¹⁸ together with the atom to which they are connected form a 3-20 membered cycloalkyl or 4-20 membered heterocyclyl ring.

29. The bivalent compound of claim 13, wherein
in FORMULA 2,
A is null; or
A is NR⁴, wherein R⁴ is selected from hydrogen, optionally substituted C₁-C₈ alkyl, optionally substituted C₂-C₈ alkenyl, optionally substituted C₂-C₈ alkynyl, optionally substituted C₁-C₈alkoxyC₁-C₈alkyl, optionally substituted C₁-C₈alkylaminoC₁-C₈alkyl, optionally substituted 3-10 membered carbocyclyl, optionally substituted 4-10 membered heterocyclyl, optionally substituted aryl, and optionally substituted heteroaryl.

30. The bivalent compound of claim 29, wherein
in FORMULA 2,
Ar—R¹ is a moiety of FORMULAE B2 or B3: 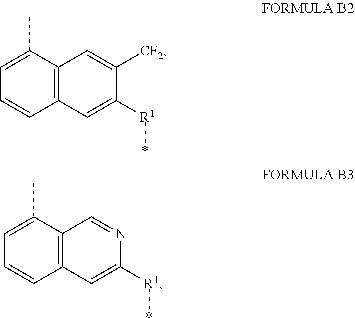
wherein
* indicates the connection to the linker moiety of the bivalent compound; and
R¹ is the same as in FORMULA 2; or,
A-Ar—R¹ is a moiety of FORMULAE B4, B5, or B6: 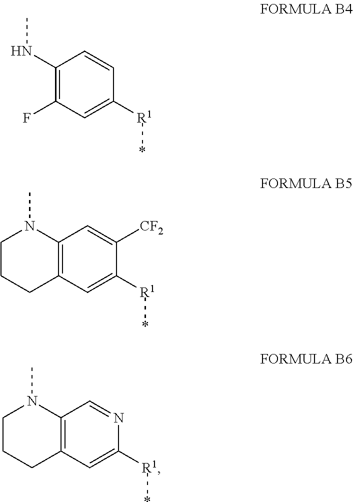
wherein
* indicates the connection to the linker moiety of the bivalent compound; and
R¹ is the same as in FORMULA 2.

31-32. (canceled)

33. The bivalent compound of claim 13, wherein
in FORMULA 2,
R¹ is selected from optionally substituted 3-10 membered carbocyclylene, optionally substituted 4-10 membered heterocyclyl, optionally substituted aryl and optionally substituted heteroaryl;
and/or
R² is selected from optionally substituted C₁-C₈ alkyl, optionally substituted 3-10 membered carbocyclyl, optionally substituted 4-10 membered heterocyclyl, optionally substituted aryl, and optionally substituted heteroaryl; and/or
R³ is selected from COR¹⁴ and CONR¹⁴R¹⁵.

34. The bivalent compound of claim 13, wherein
in FORMULA 2,
R¹ is selected from optionally substituted aryl and optionally substituted heteroaryl.

35. The bivalent compound of claim 13, wherein
in FORMULA 2,
R¹ is selected from optionally substituted pyrazole and optionally substituted pyridinyl; and/or R³ is selected from COMe and CONHMe.

36-38. (canceled)

39. The bivalent compound of claim 12, wherein the CBP/P300 ligand is derived from any of the following: 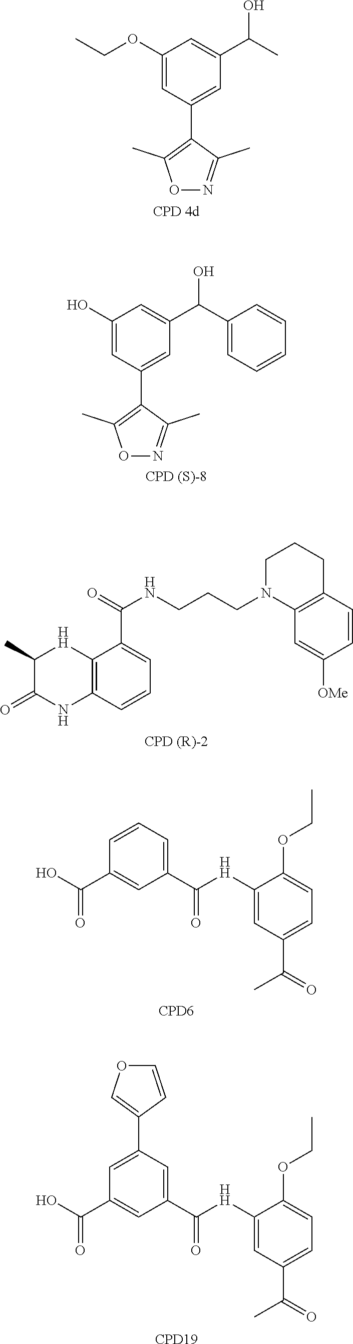 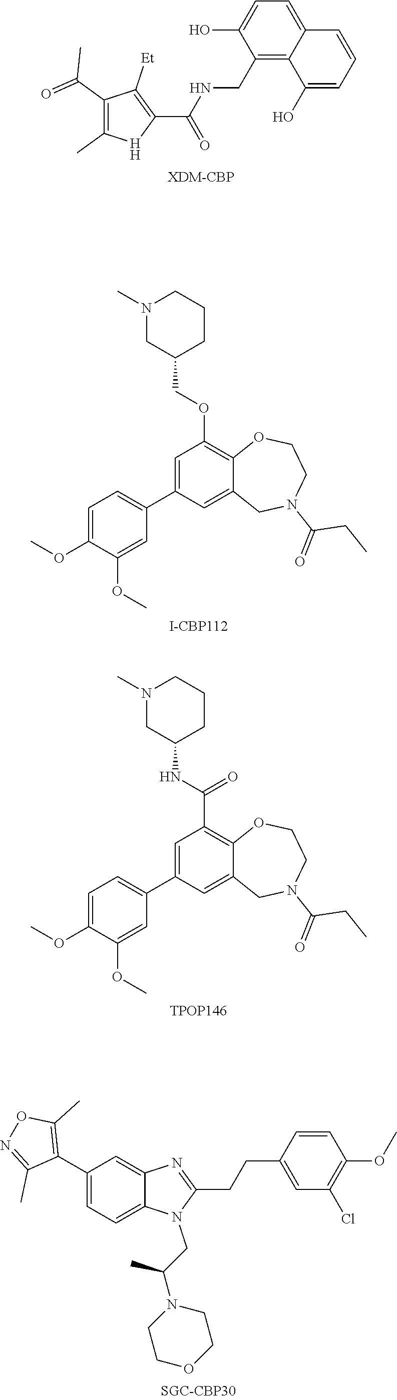 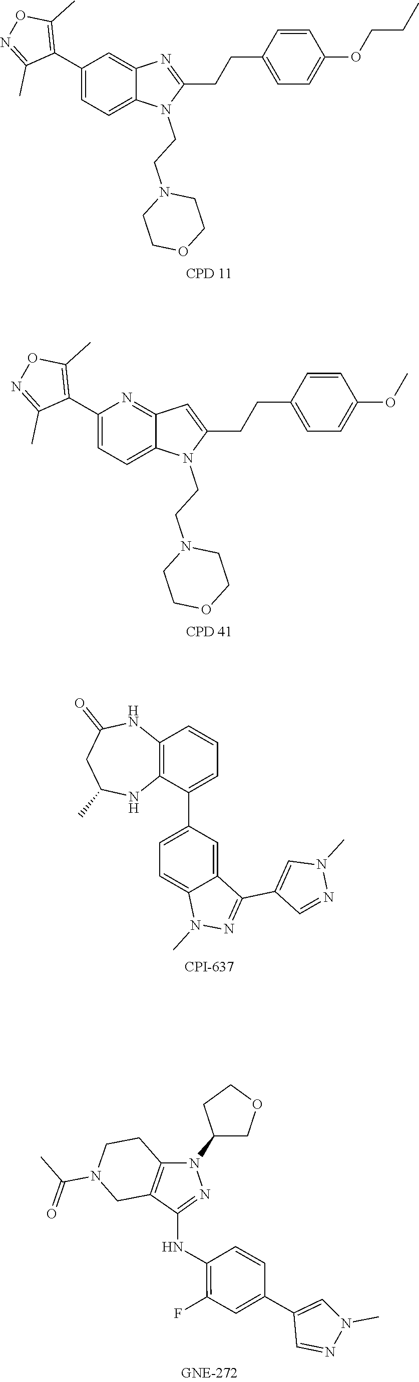 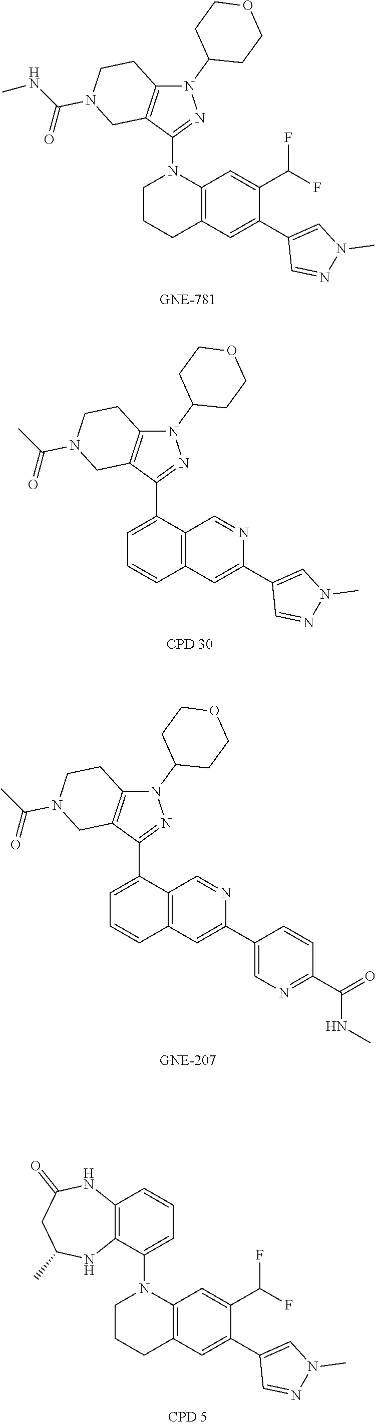 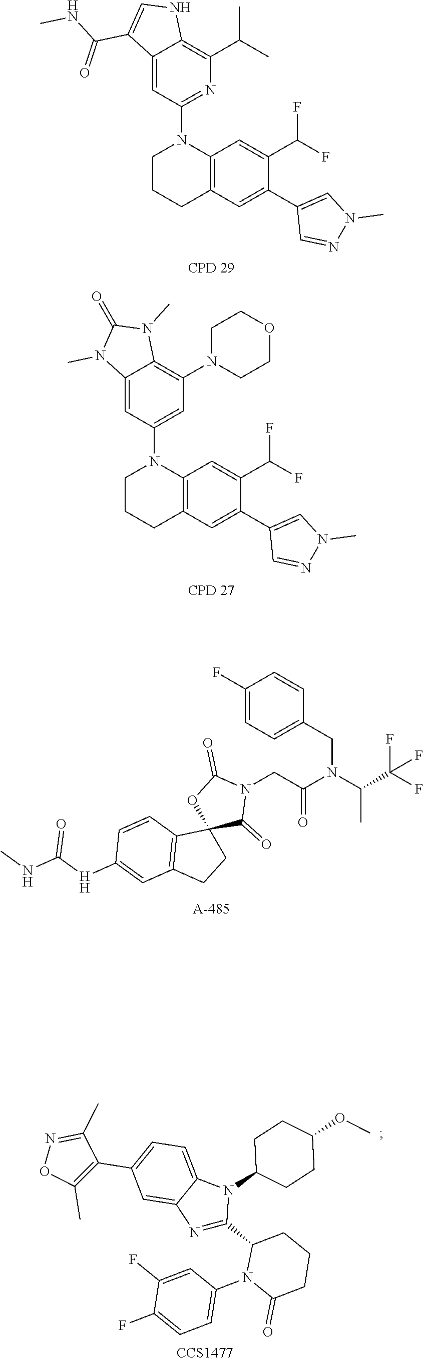
or, the CBP/P300 ligand is derived from any of the following CBP/P300 inhibitors: C646, naphthol-AS-E, compound 1-10, MYBMIM, CCS1477, ICG-001, YH249, YH250, HBS1, OHM1, and KCN1.

40. (canceled)

41. The bivalent compound of claim 12, wherein the CBP/P300 ligand is selected from the group consisting of: 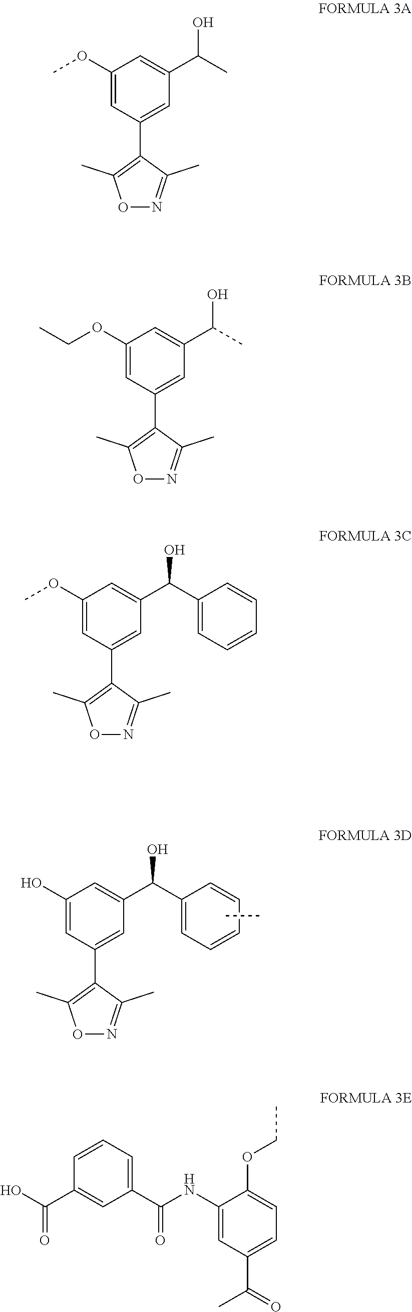 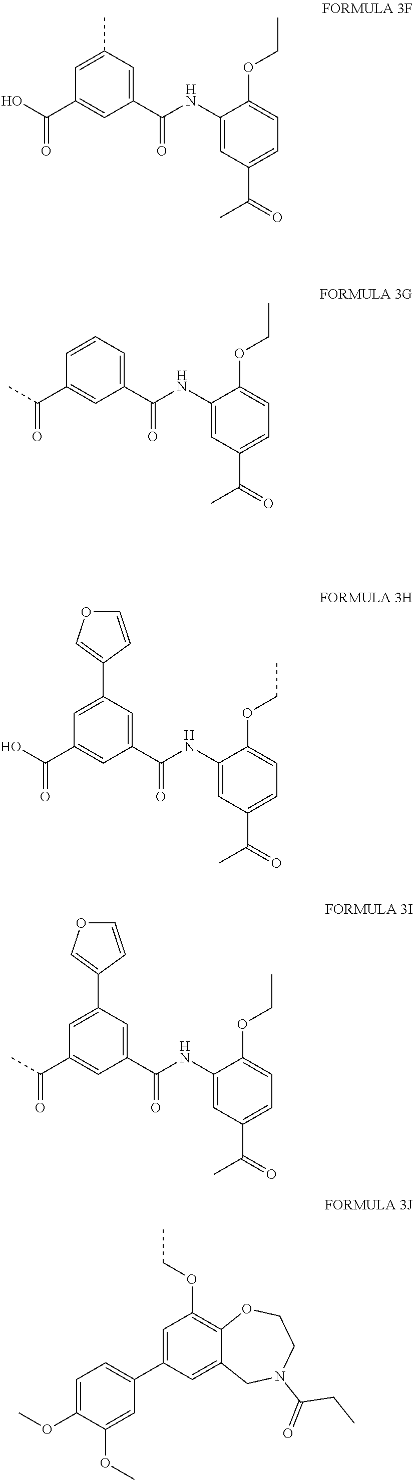 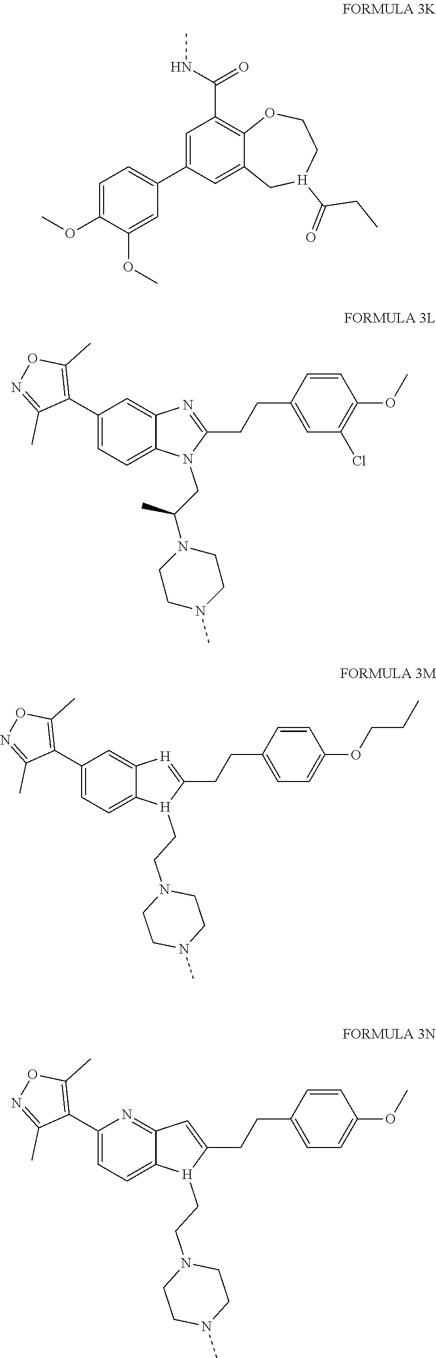 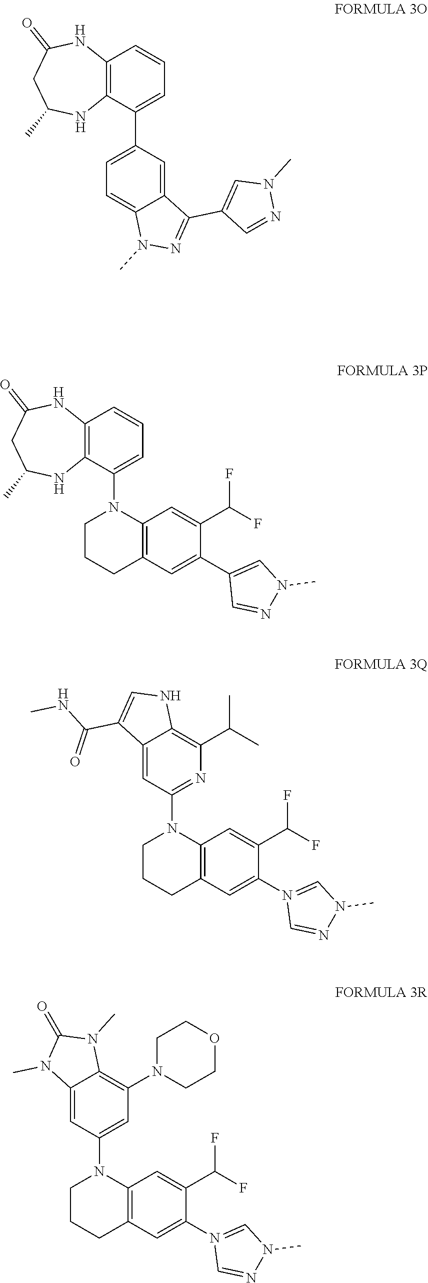 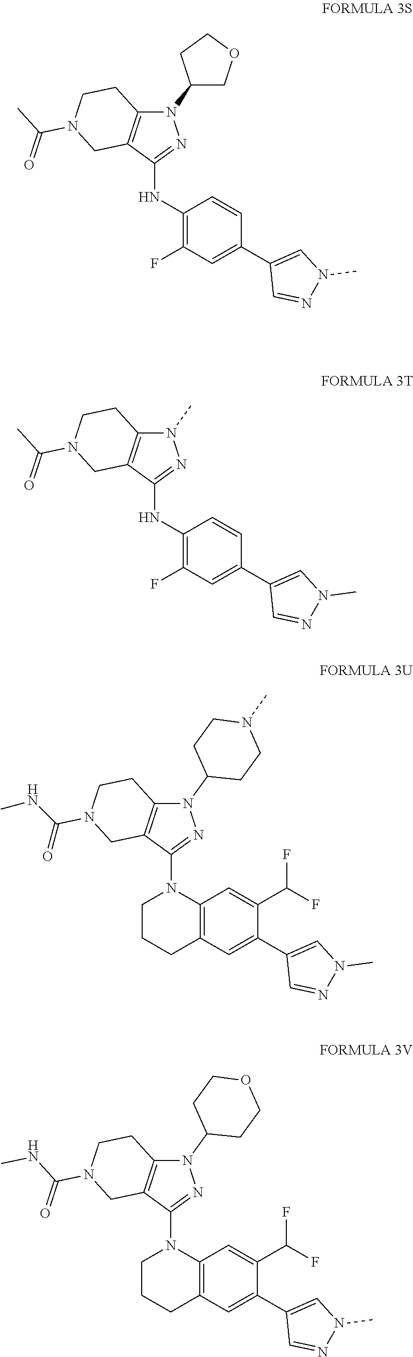 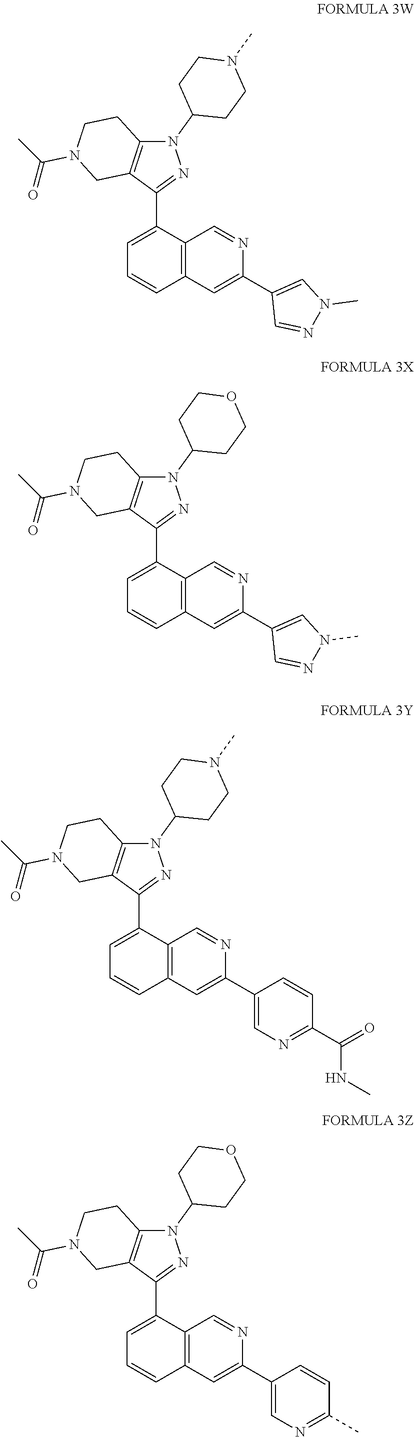 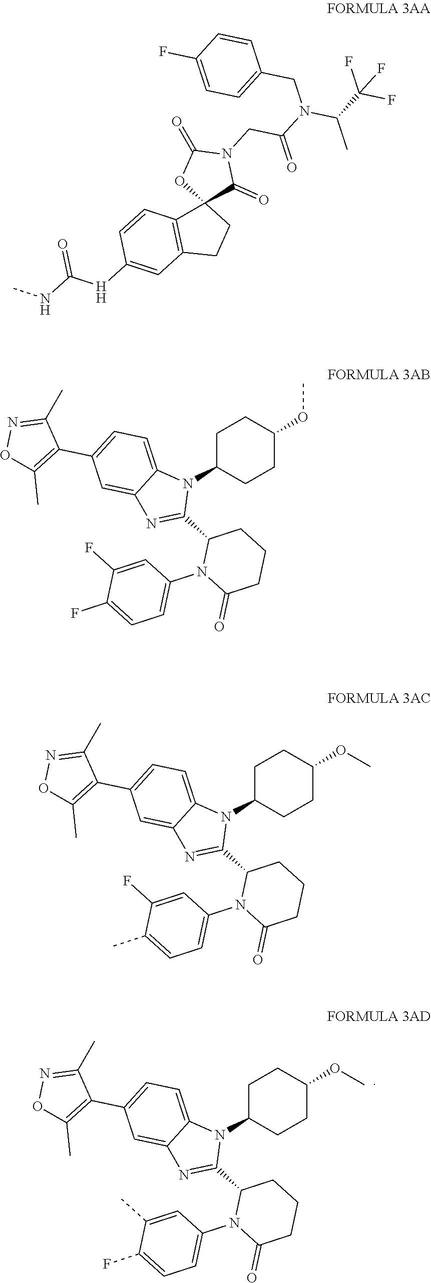
wherein the dashed bond “ [symbol] ” indicates the connection to the linker moiety of the bivalent compound

42-45. (canceled)

46. The bivalent compound of claim 12, wherein the degradation tag is derived from any of the following:

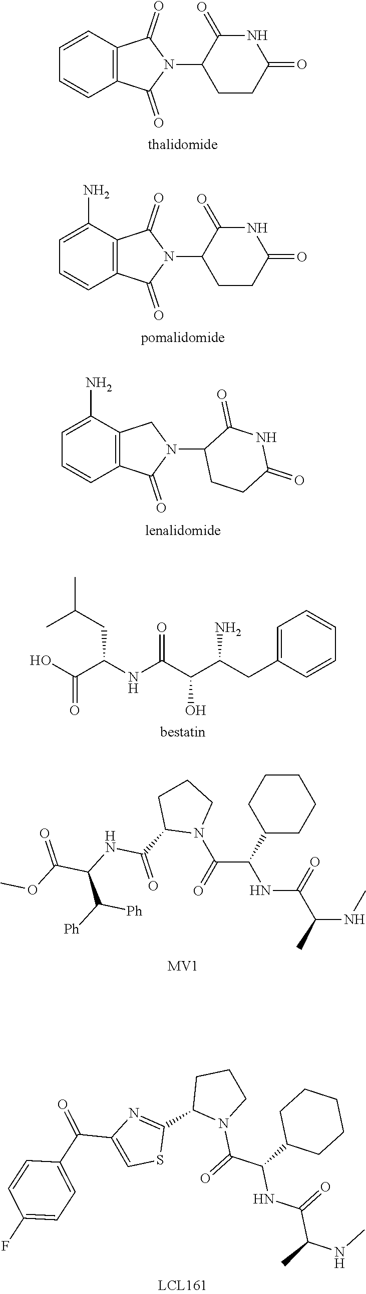

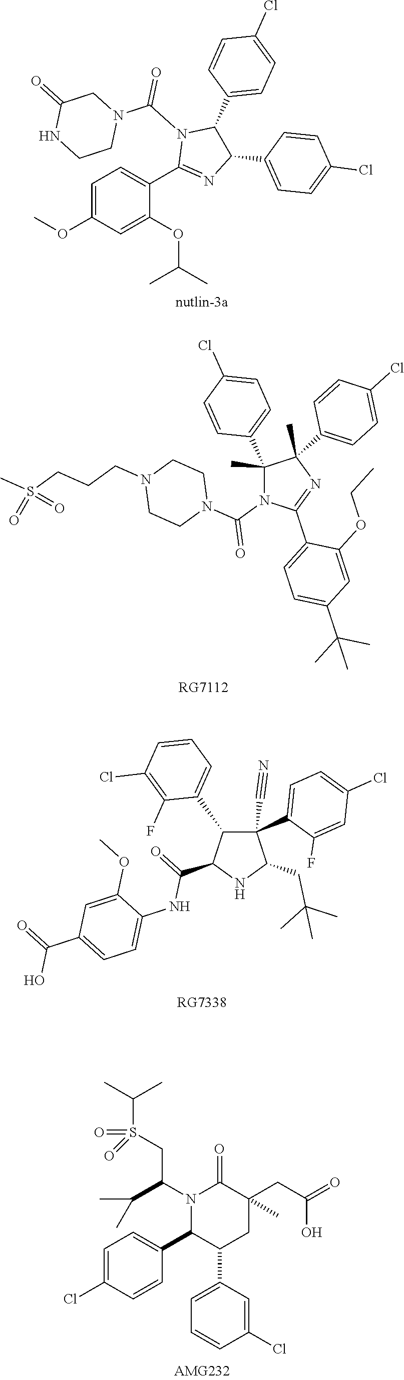

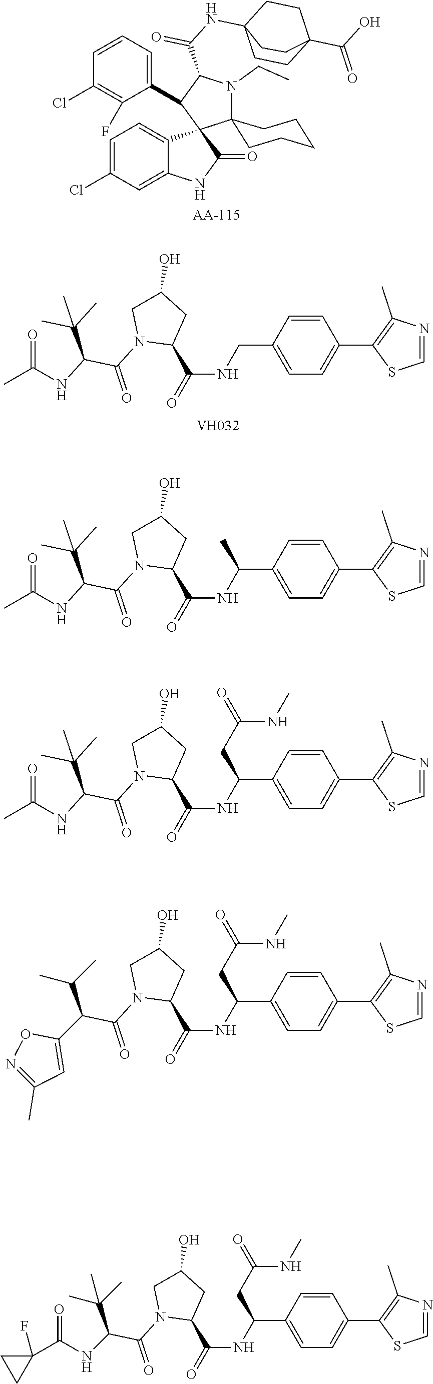

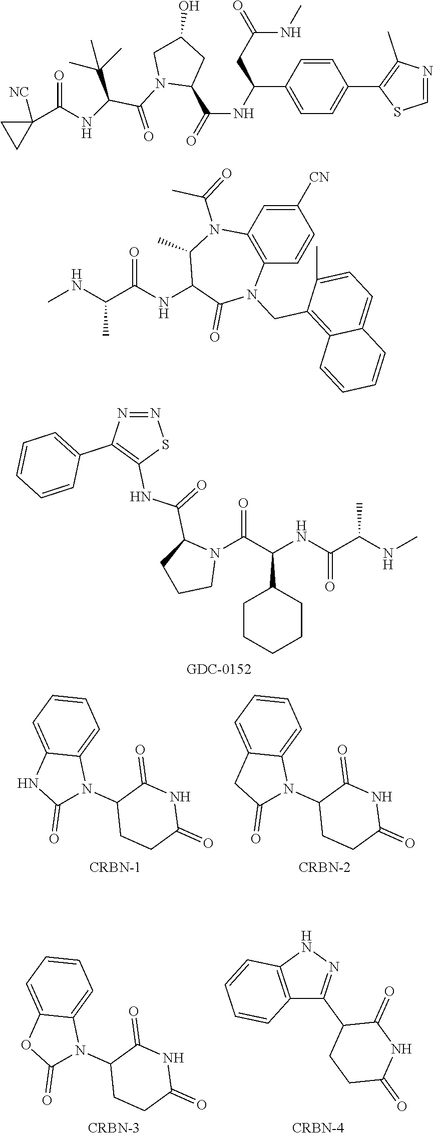

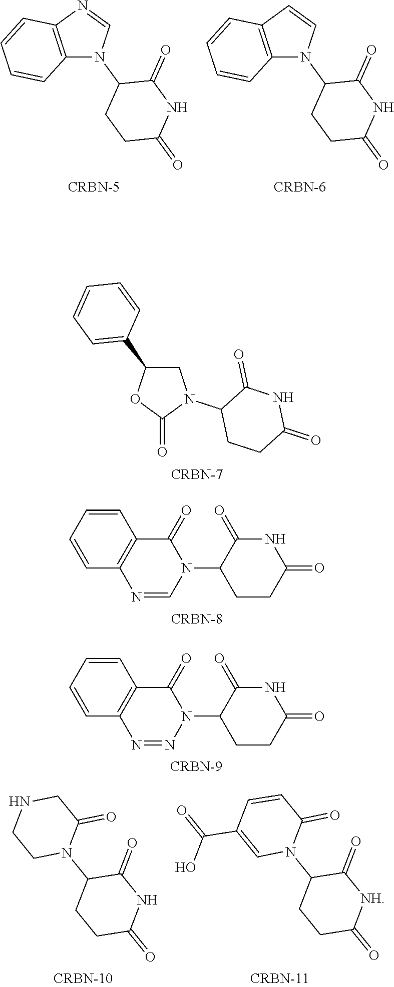

47. The bivalent compound of claim 12, wherein the degradation tag is selected from the group consisting of: 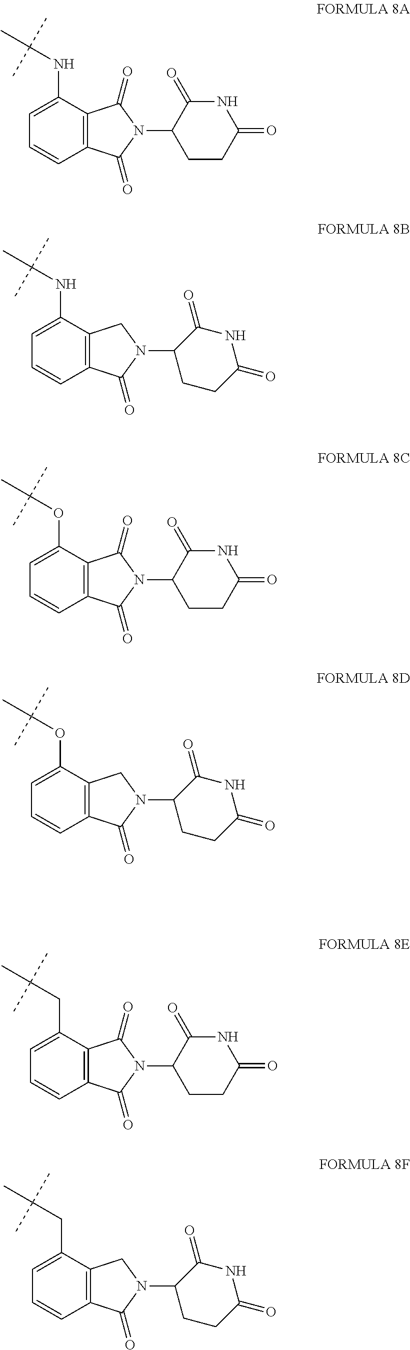 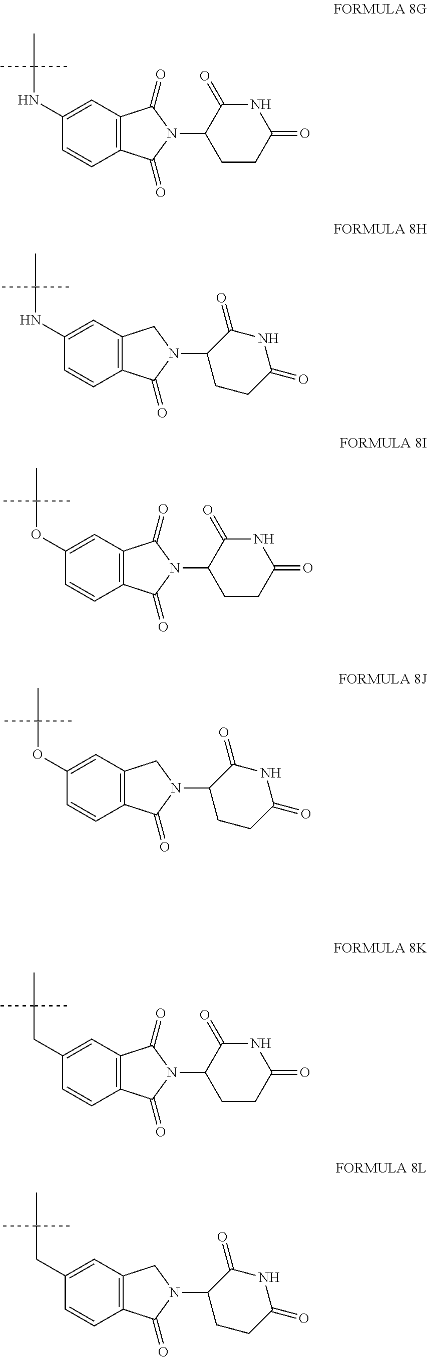 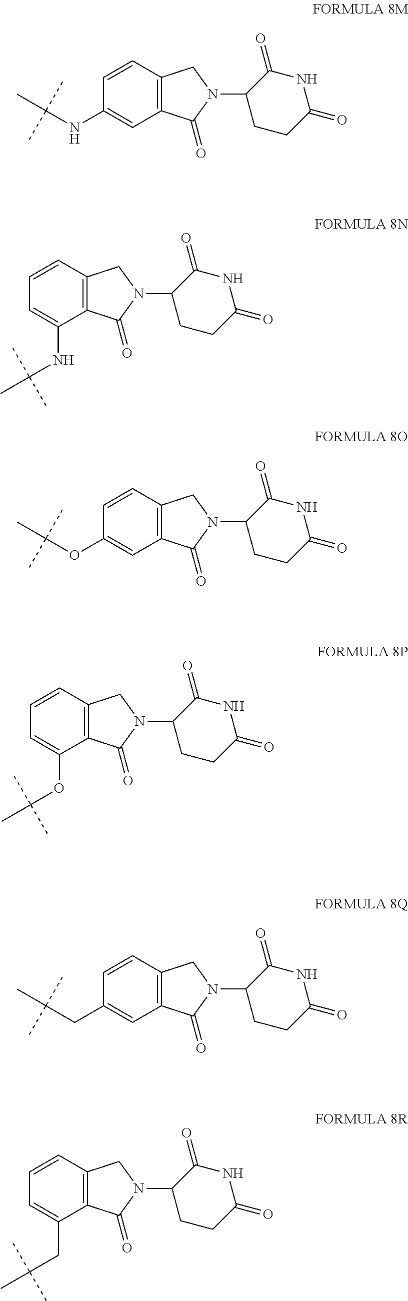 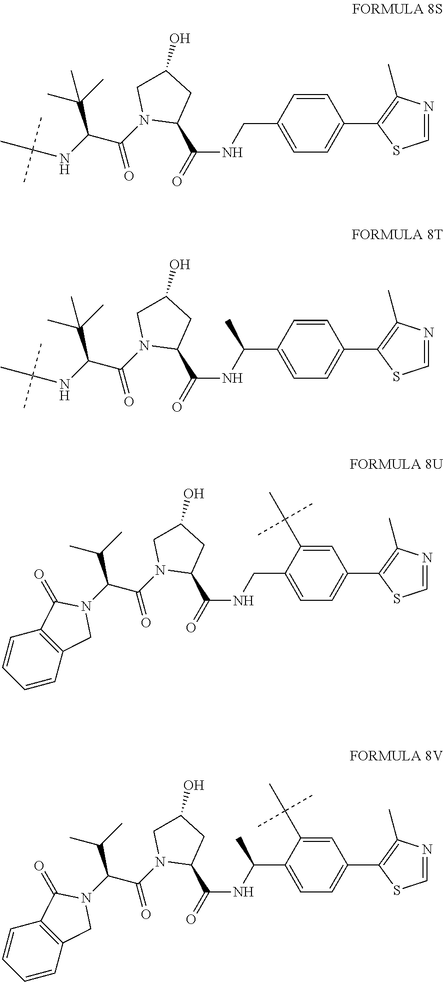 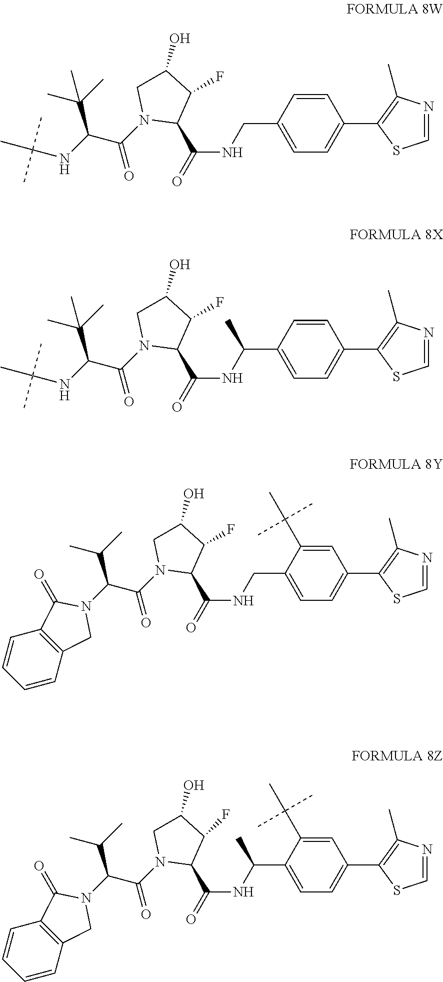 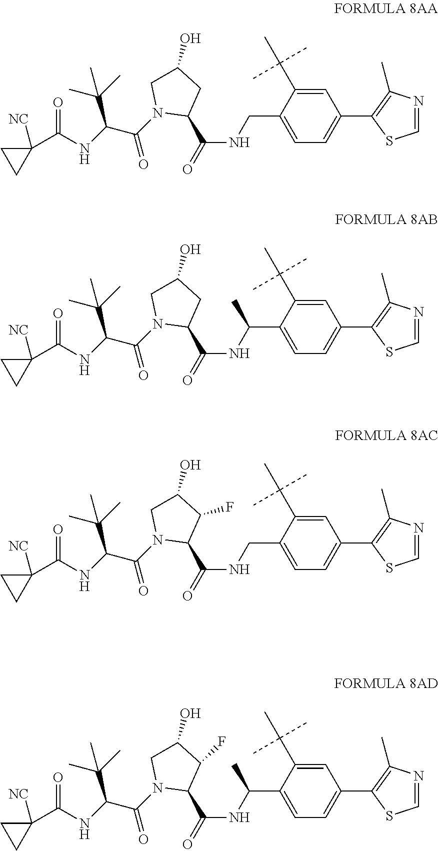 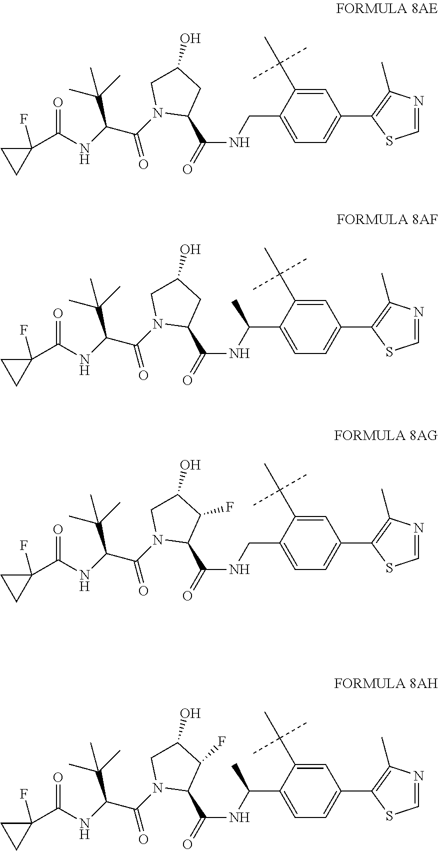 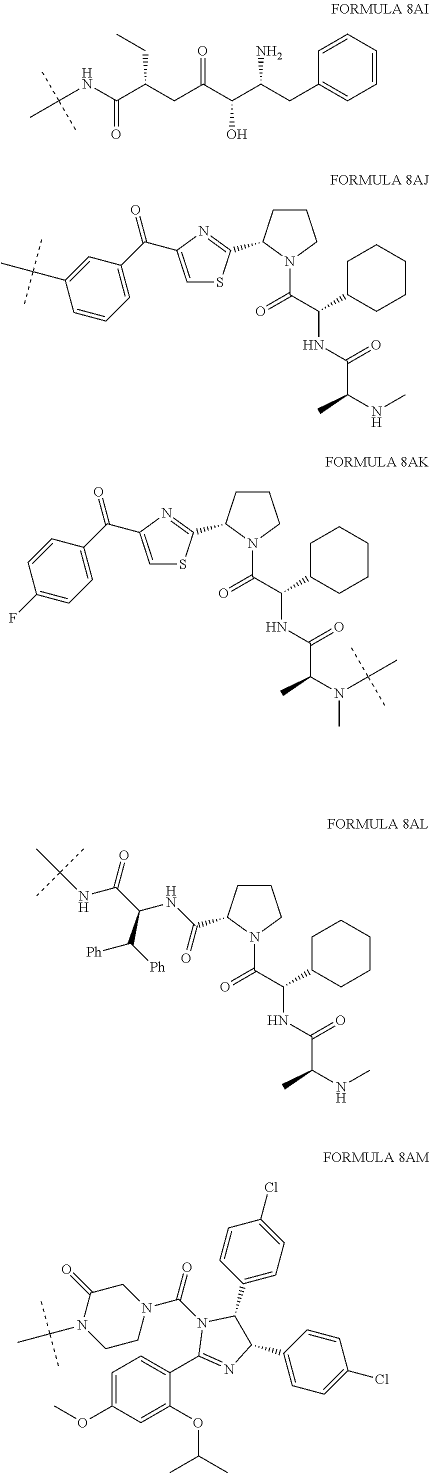 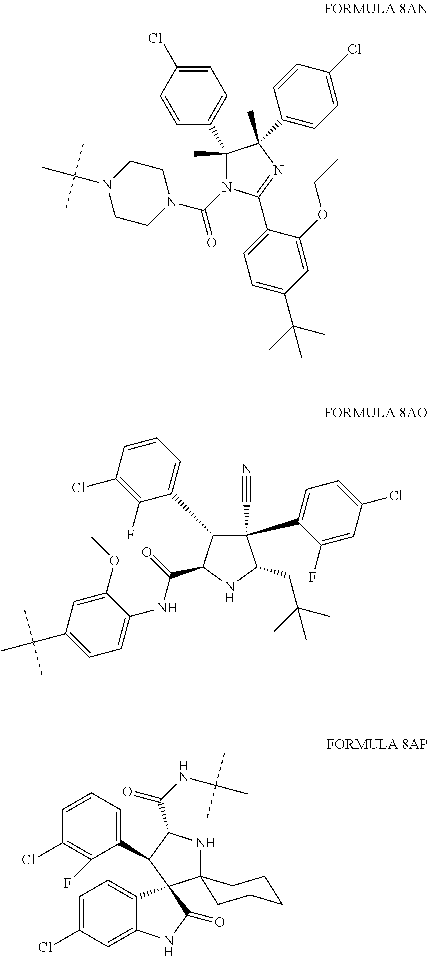 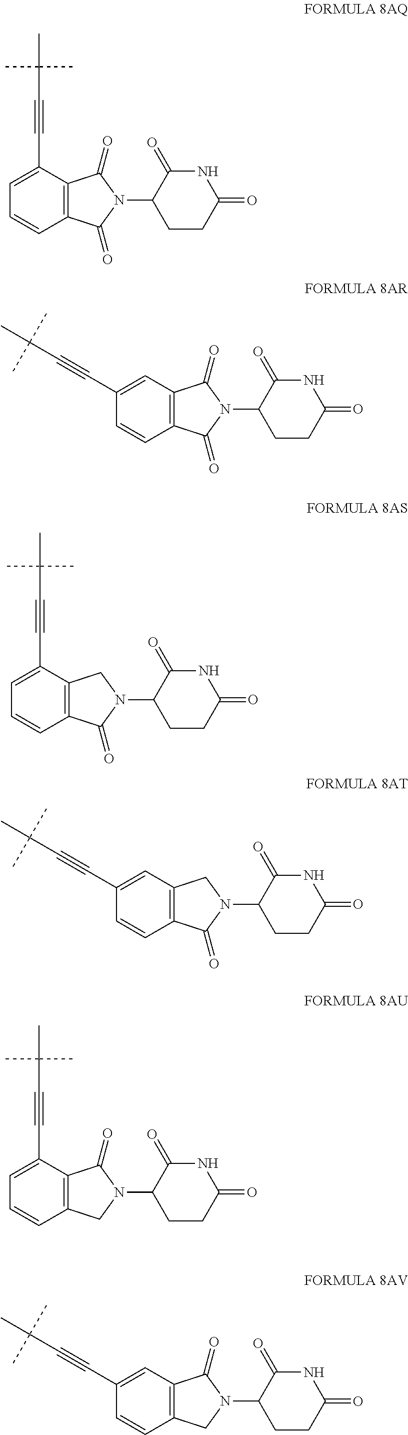 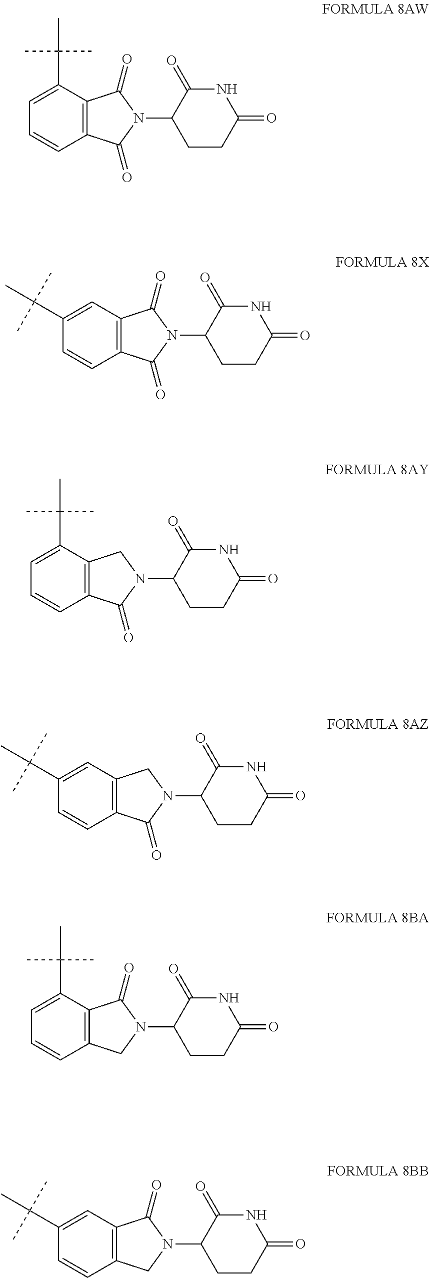 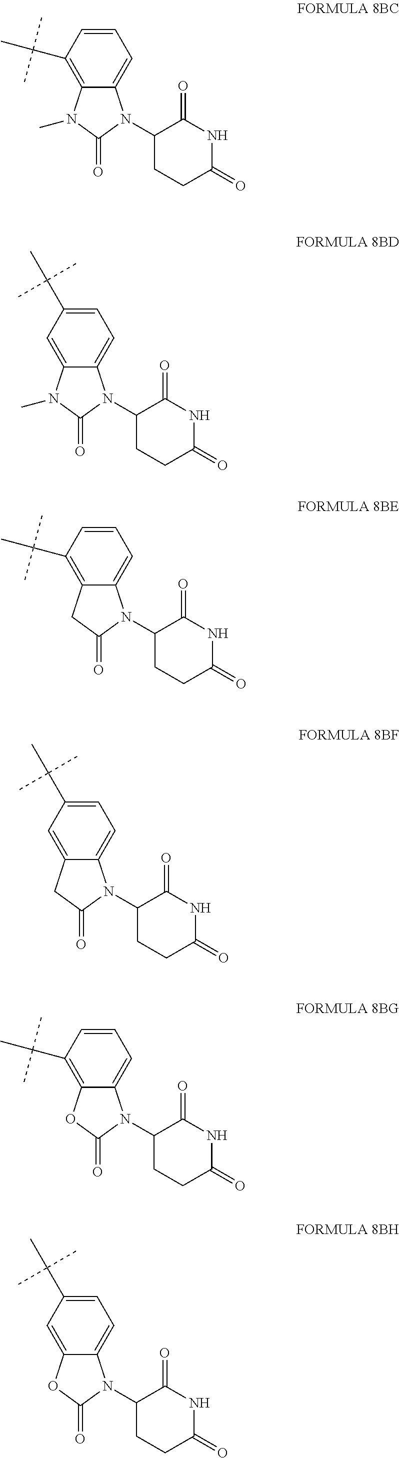 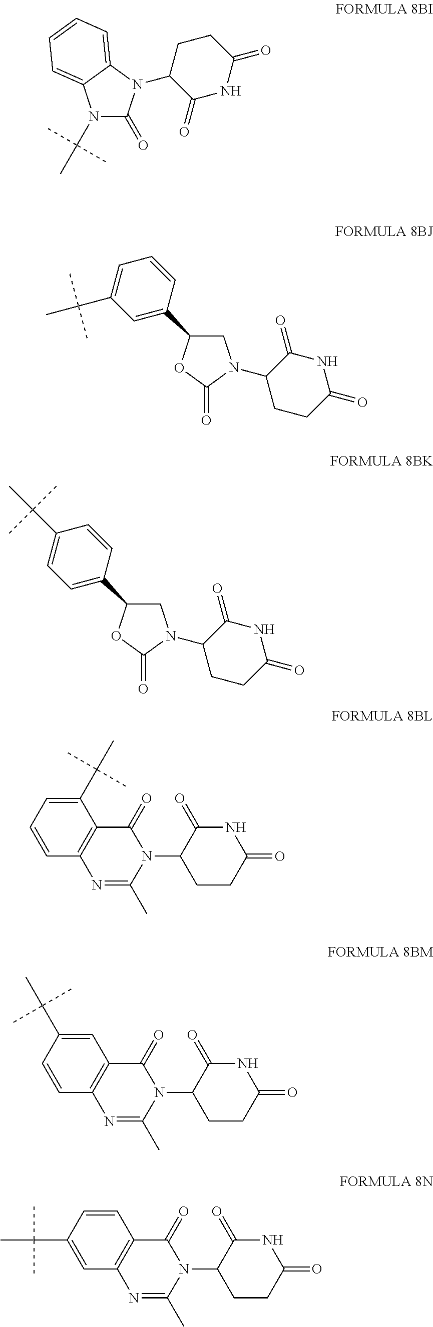 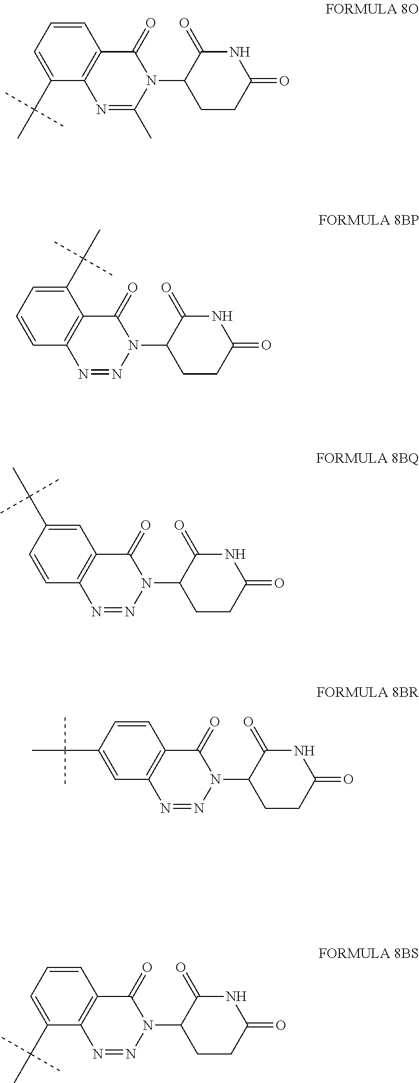 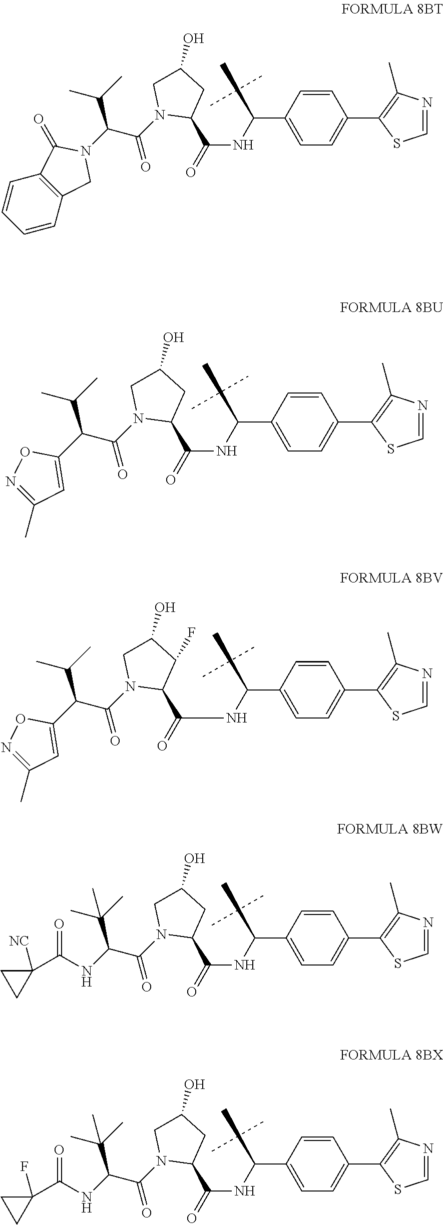 [SMILES: Cc1ncsc1-c1ccc([C@H](C)NC(=O)[C@@H]2C[C@@H](O)CN2C(=O)[C@H](C(C)C)N2Cc3ccccc3C2=O)cc1 ; Cc1cc([C@H](C(=O)N2C[C@H](O)C[C@H]2C(=O)N[C@@H](C)c2ccc(-c3scnc3C)cc2)C(C)C)on1 ; Cc1cc([C@H](C(=O)N2C[C@H](O)[C@H](F)[C@H]2C(=O)N[C@@H](C)c2ccc(-c3scnc3C)cc2)C(C)C)on1 ; Cc1ncsc1-c1ccc([C@H](C)NC(=O)[C@@H]2C[C@@H](O)CN2C(=O)[C@@H](NC(=O)C2(C#N)CC2)C(C)(C)C)cc1 ; Cc1ncsc1-c1ccc([C@H](C)NC(=O)[C@@H]2C[C@@H](O)CN2C(=O)[C@@H](NC(=O)C2(F)CC2)C(C)(C)C)cc1] 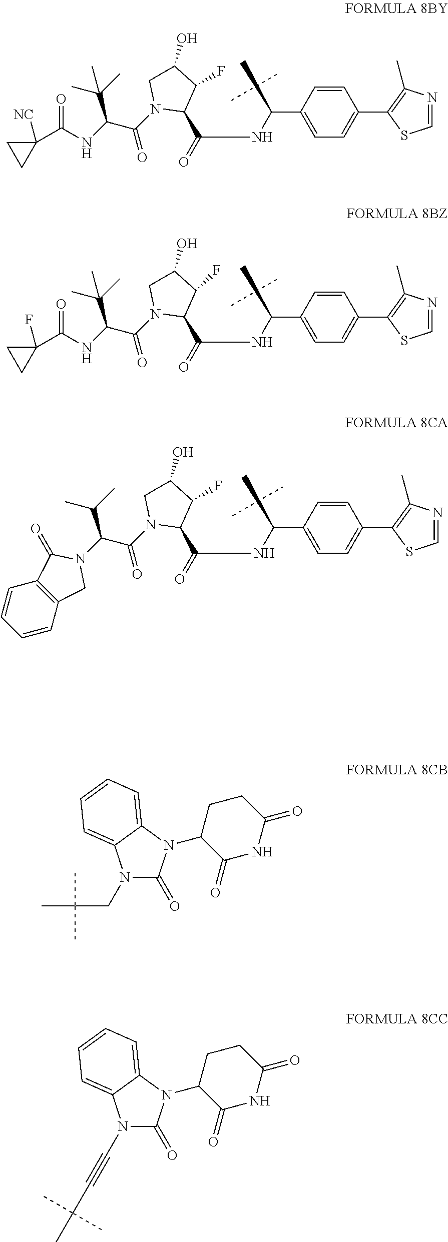 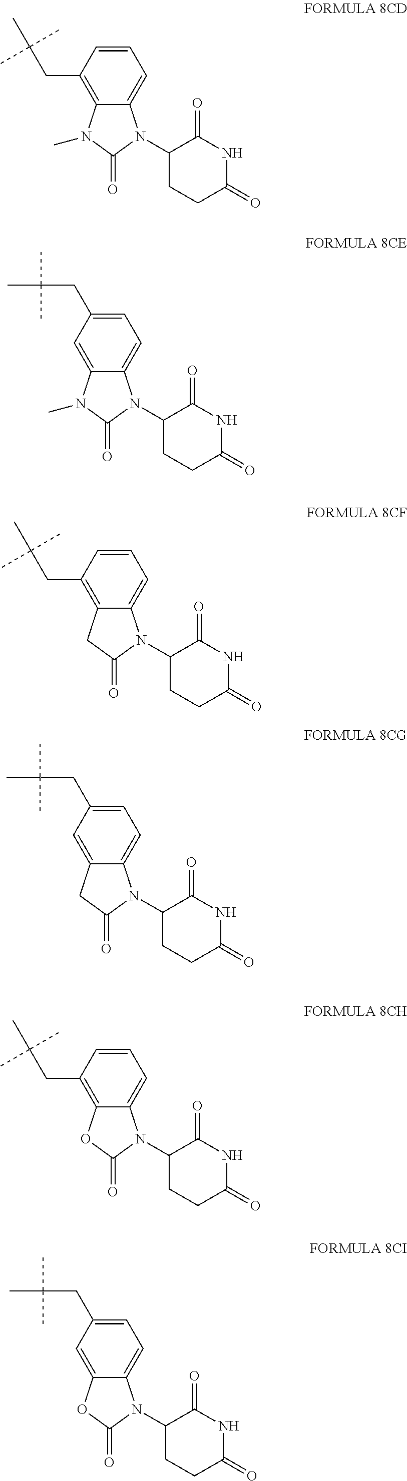 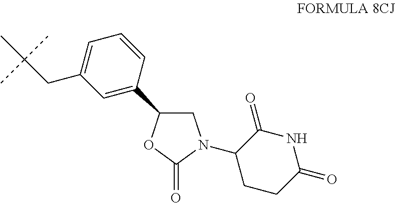 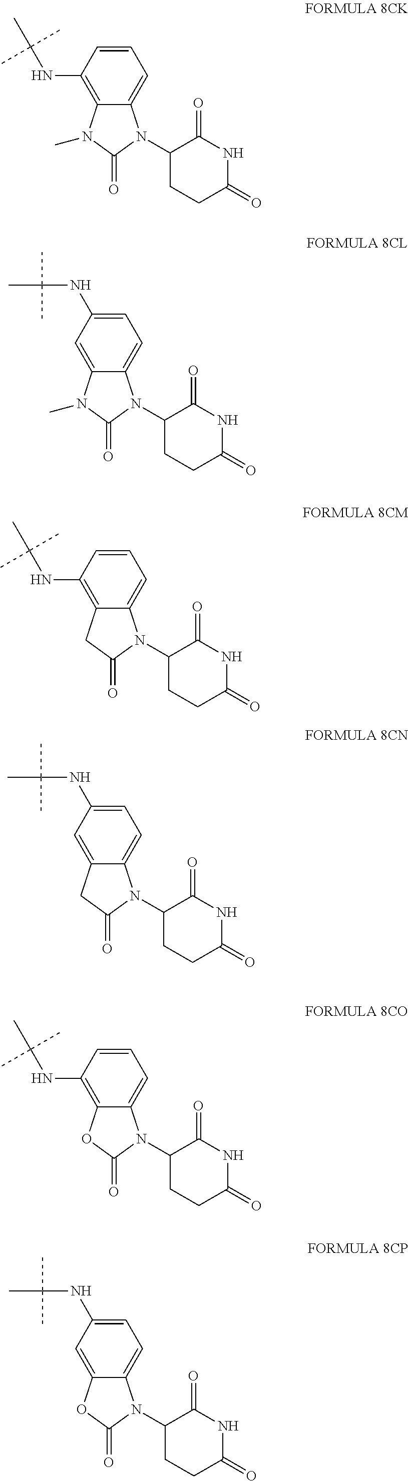 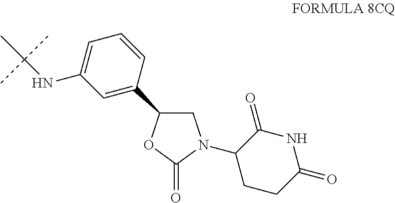 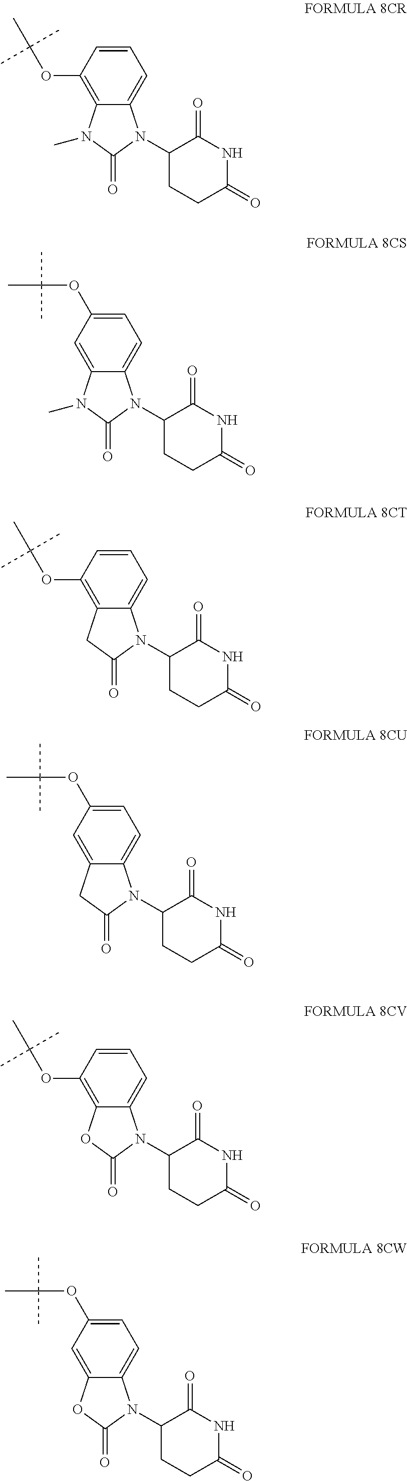 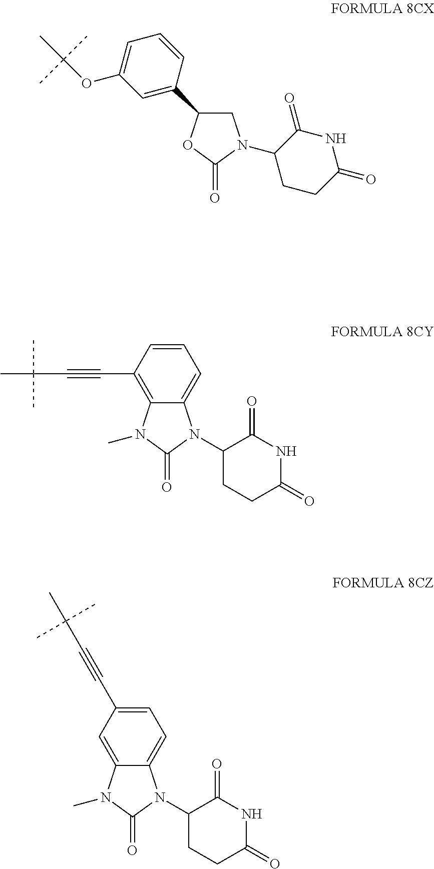 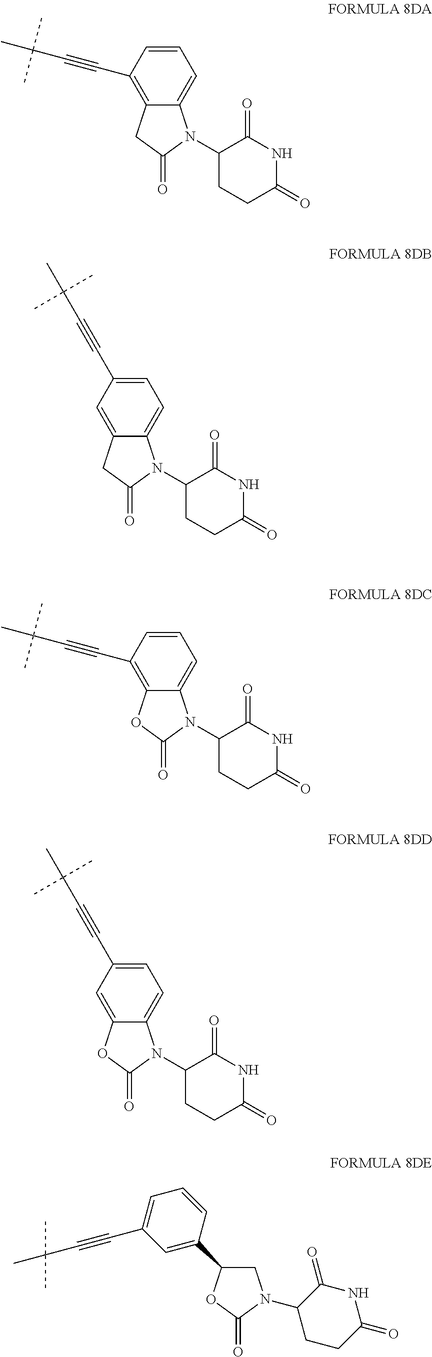 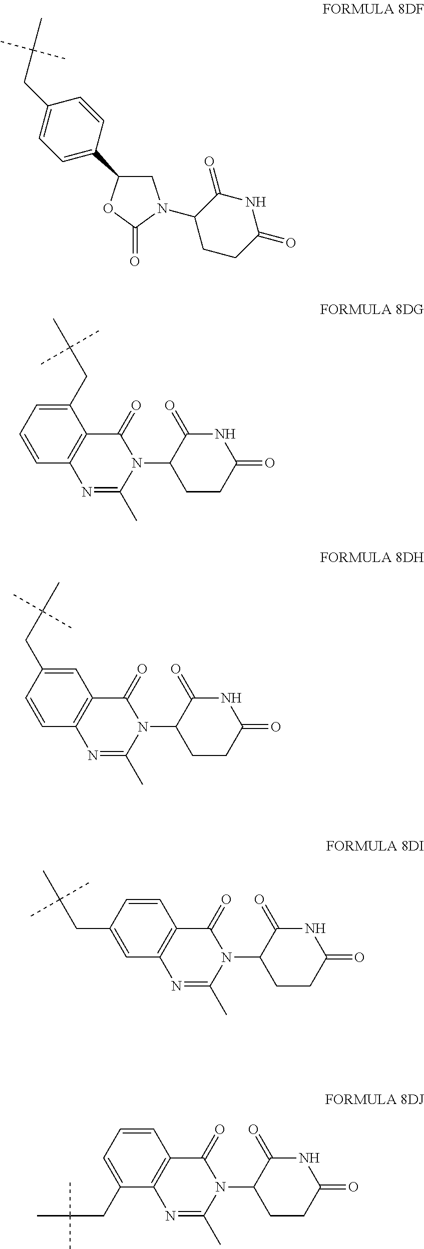 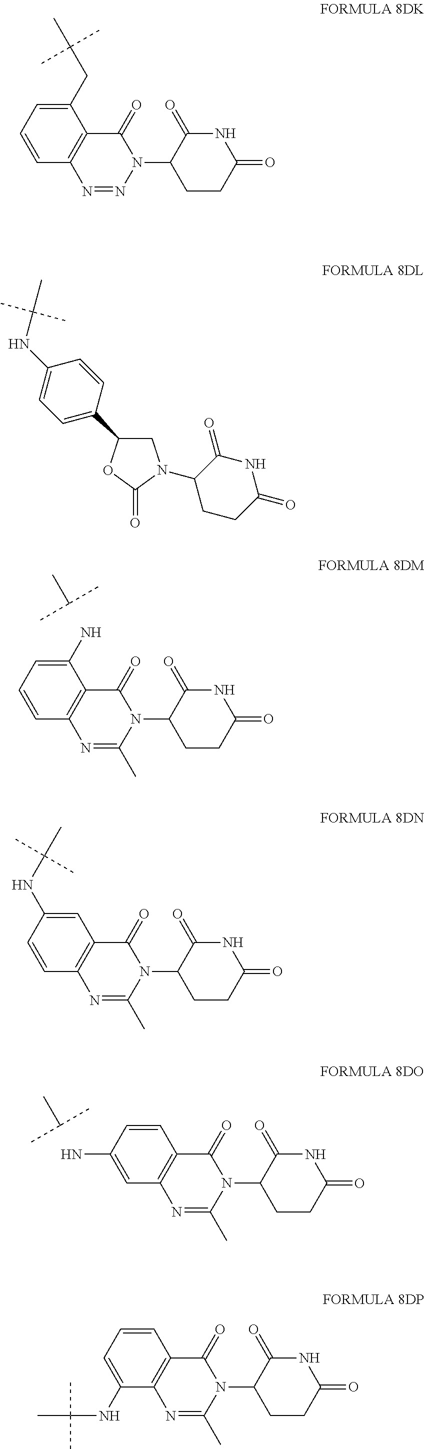 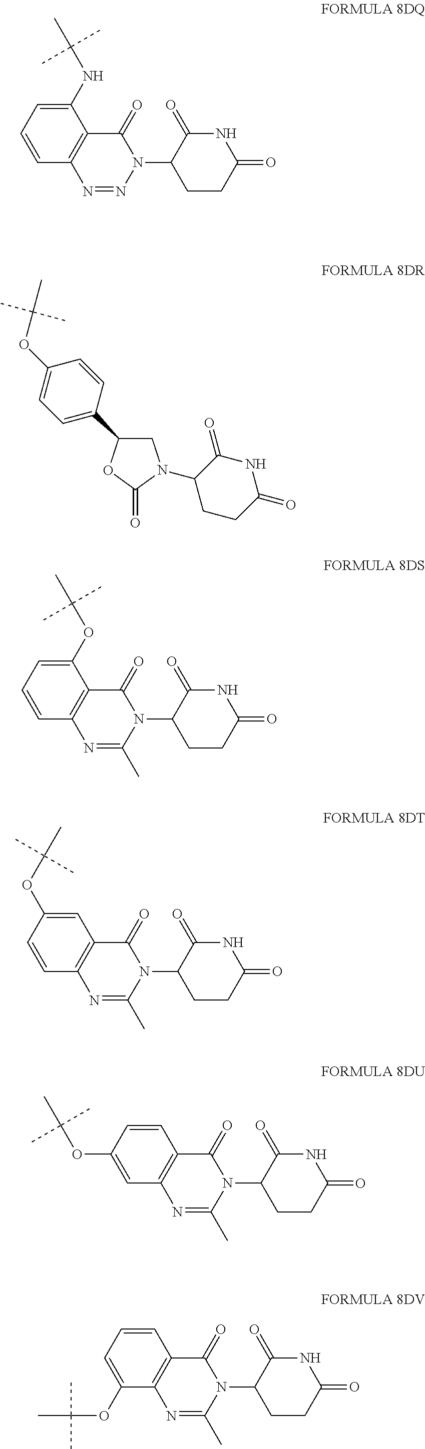 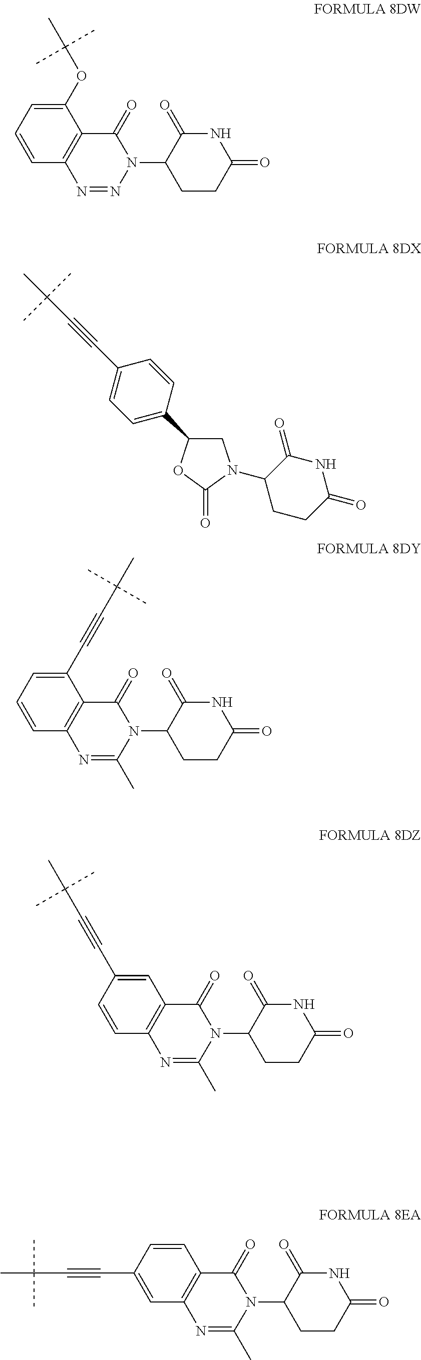 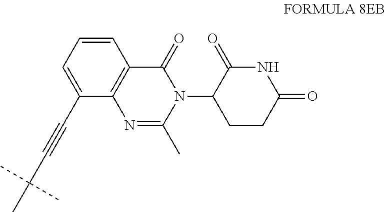 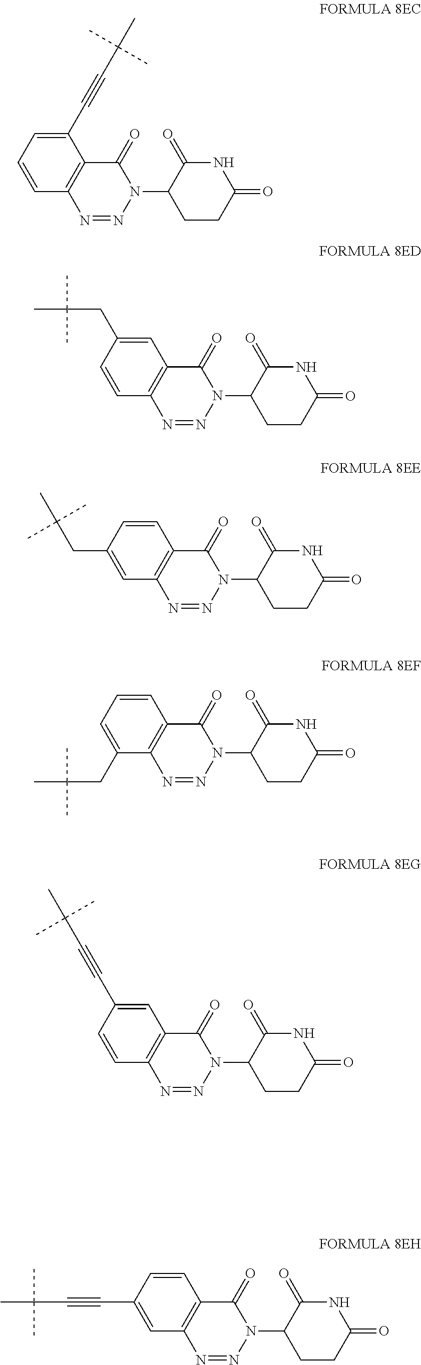 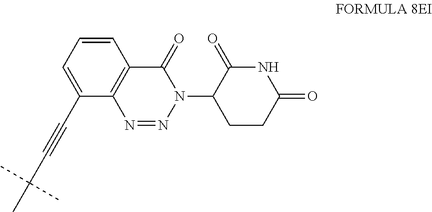 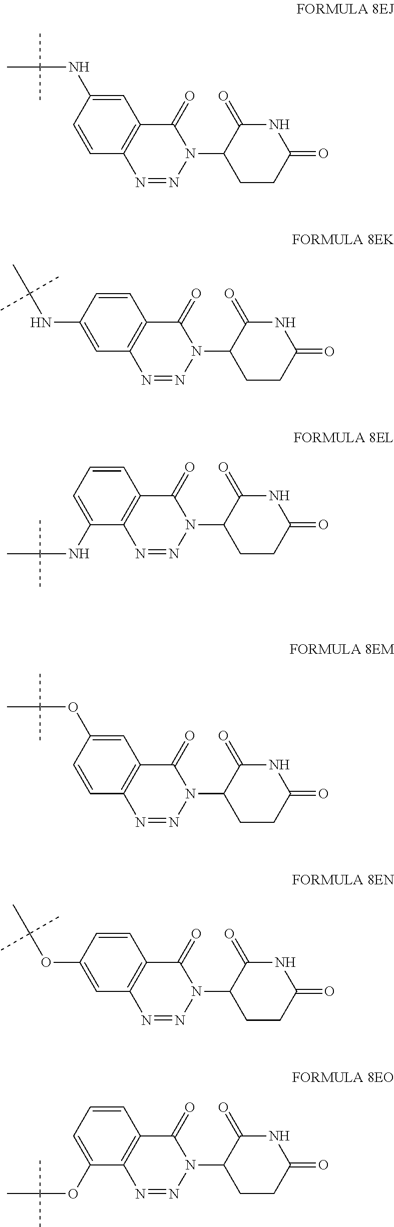 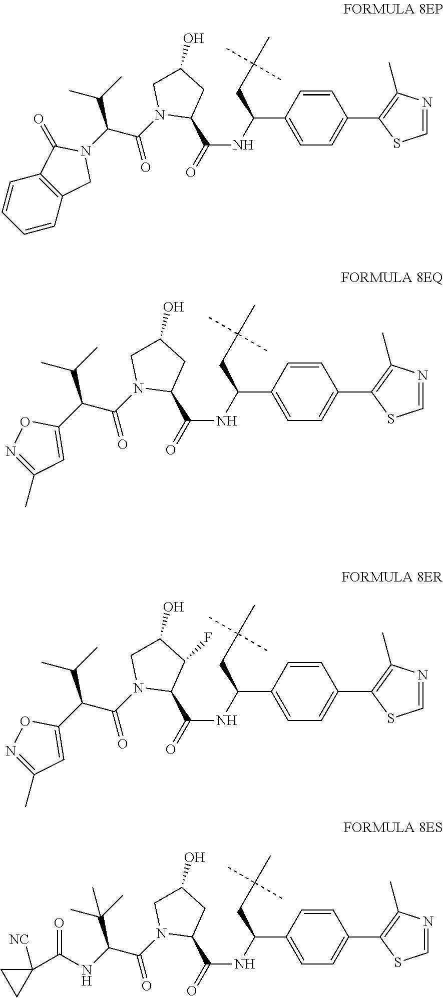 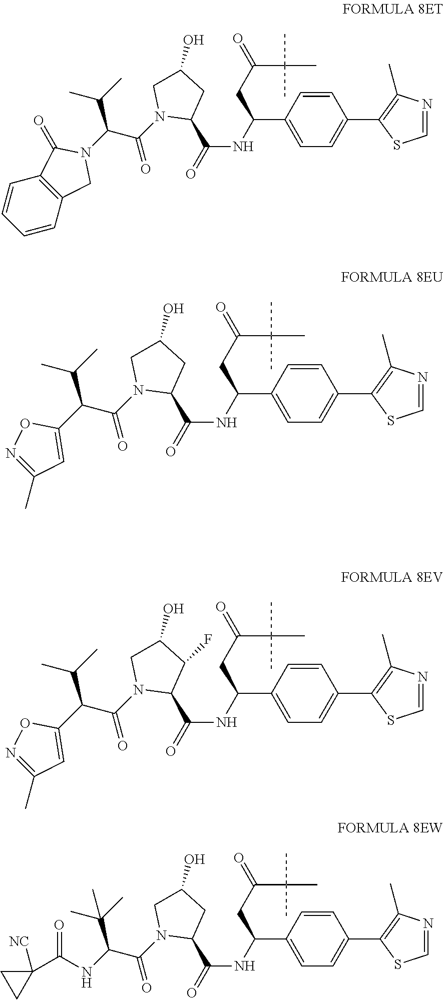 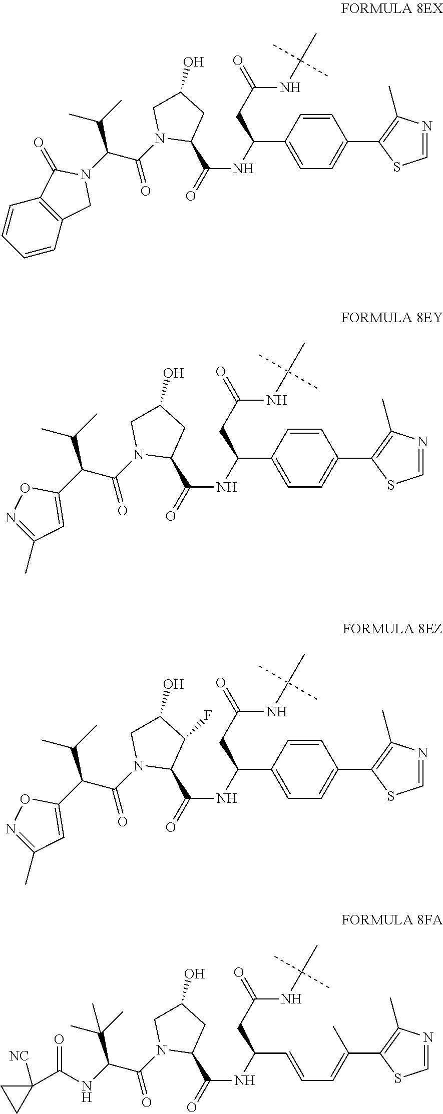 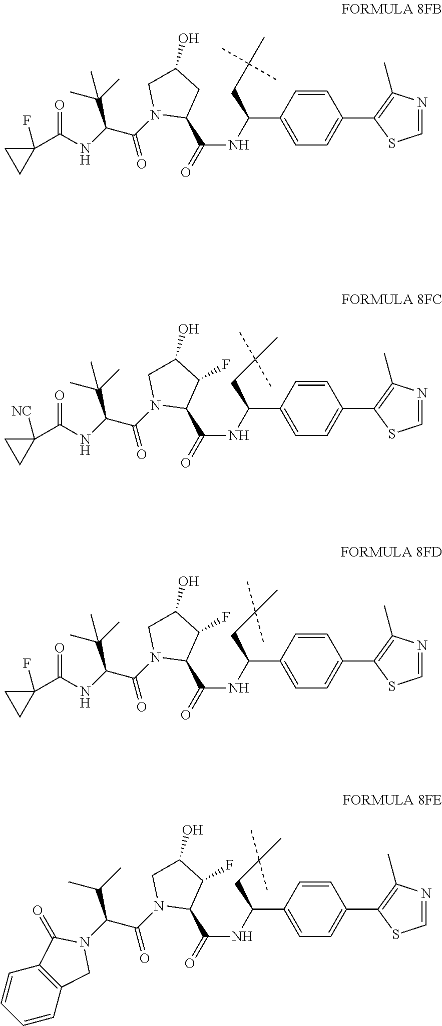 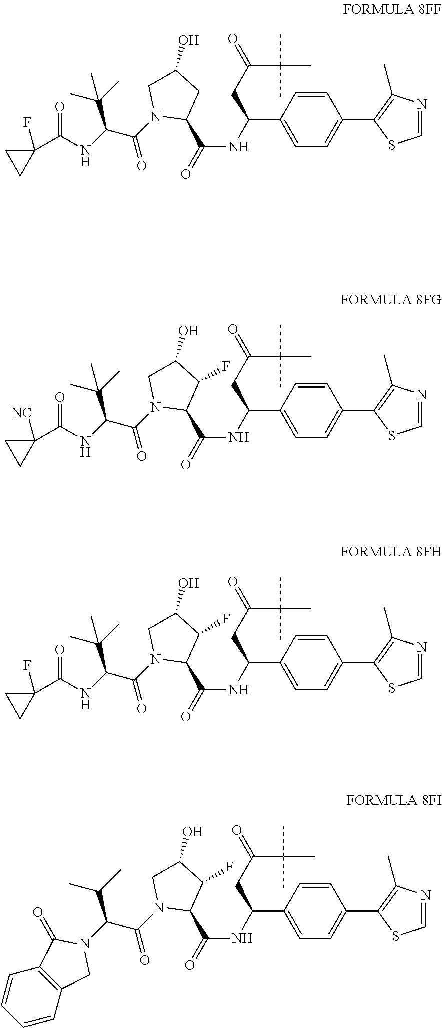 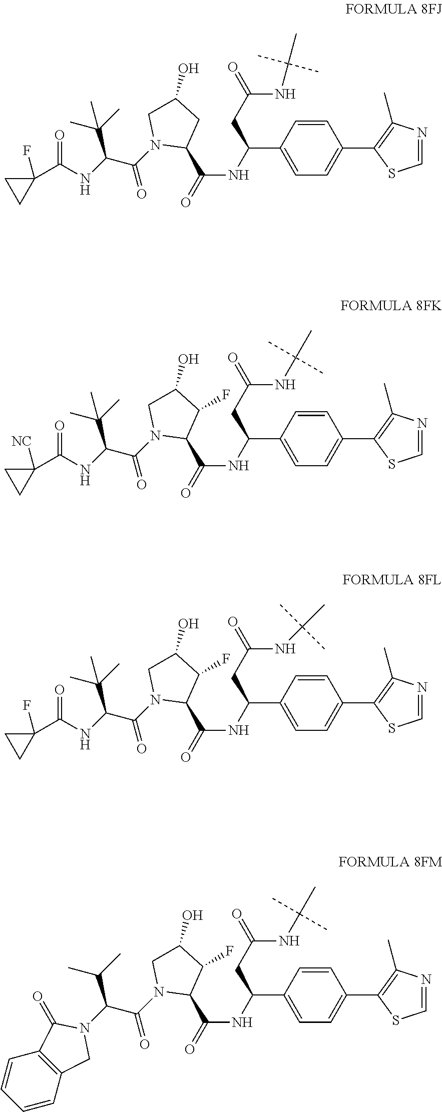 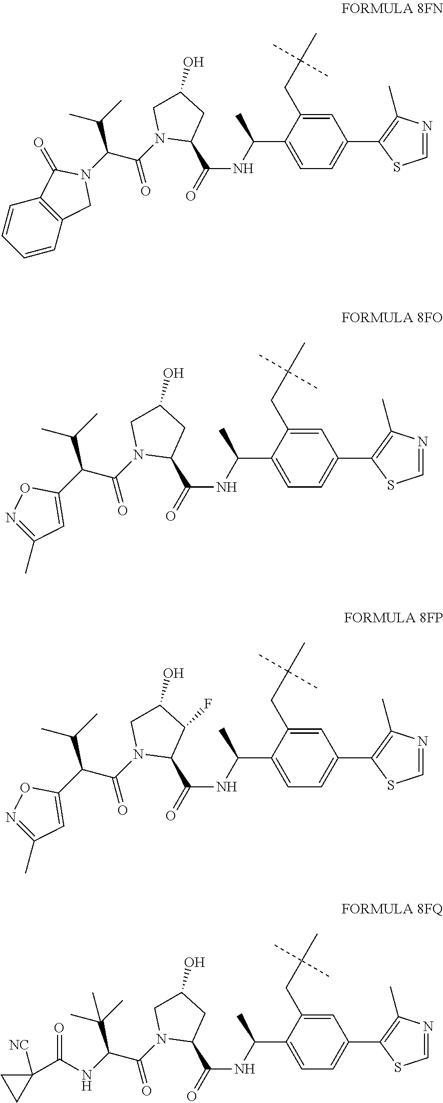 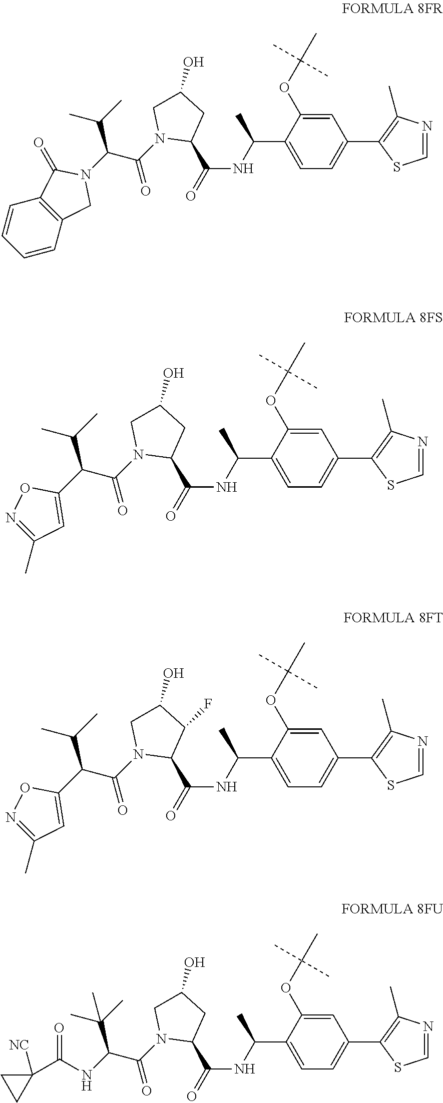 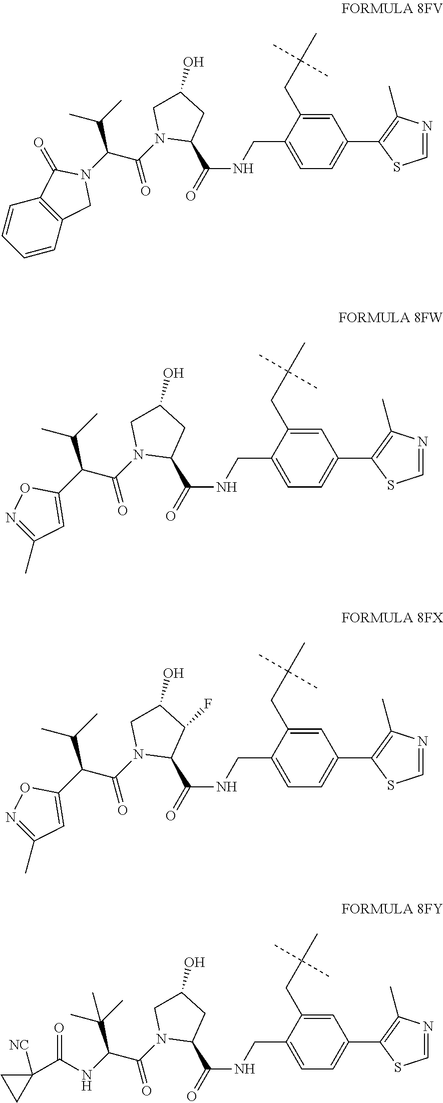 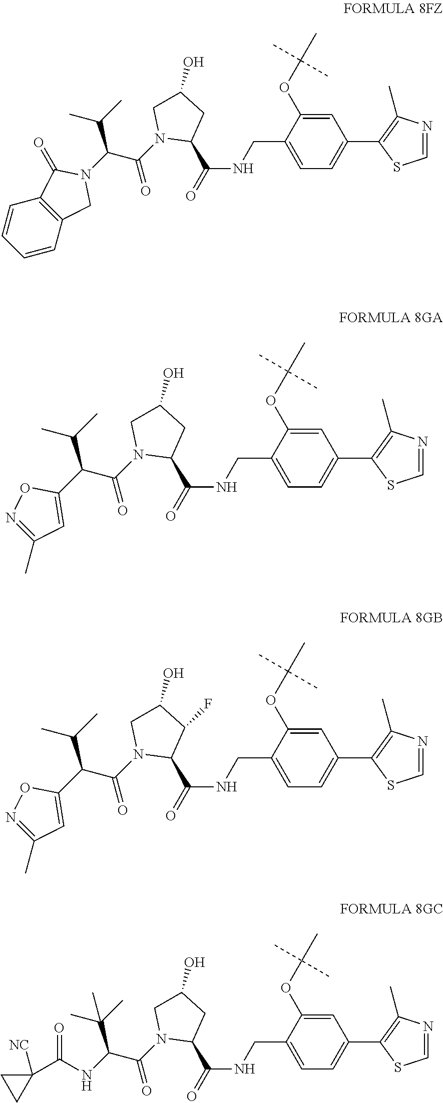 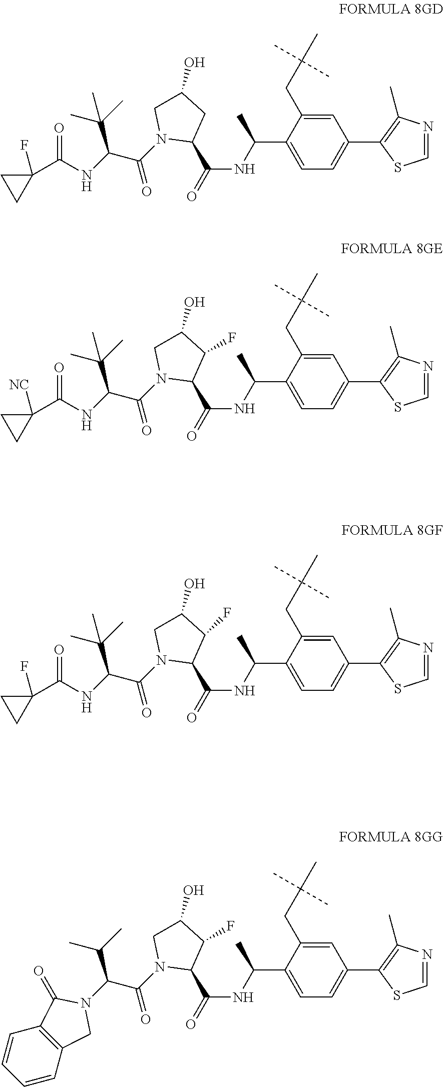 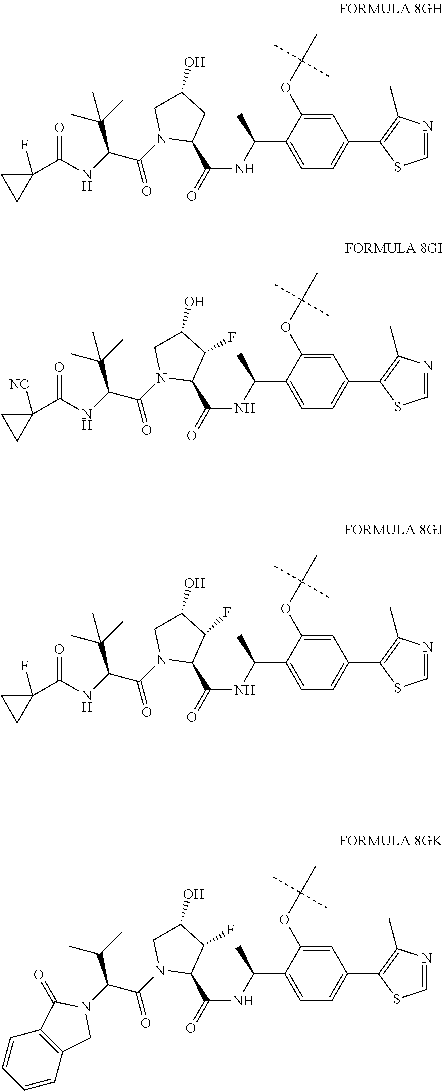 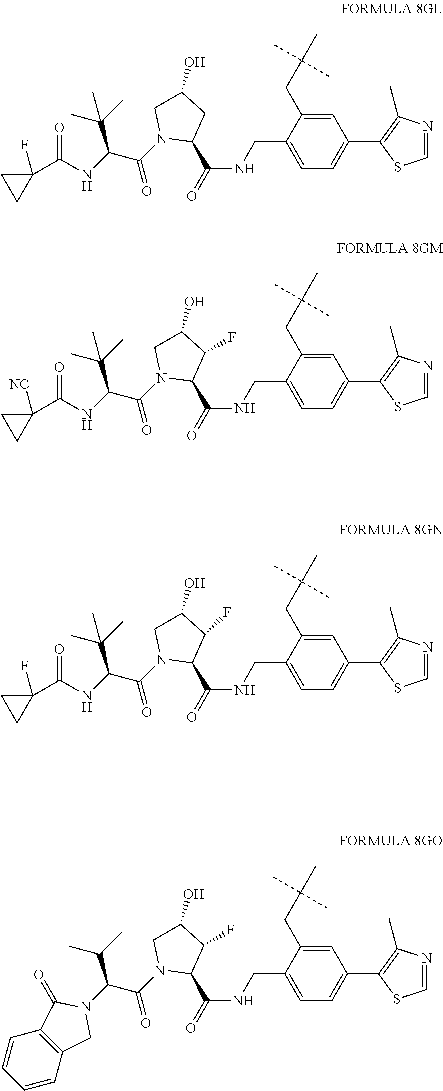 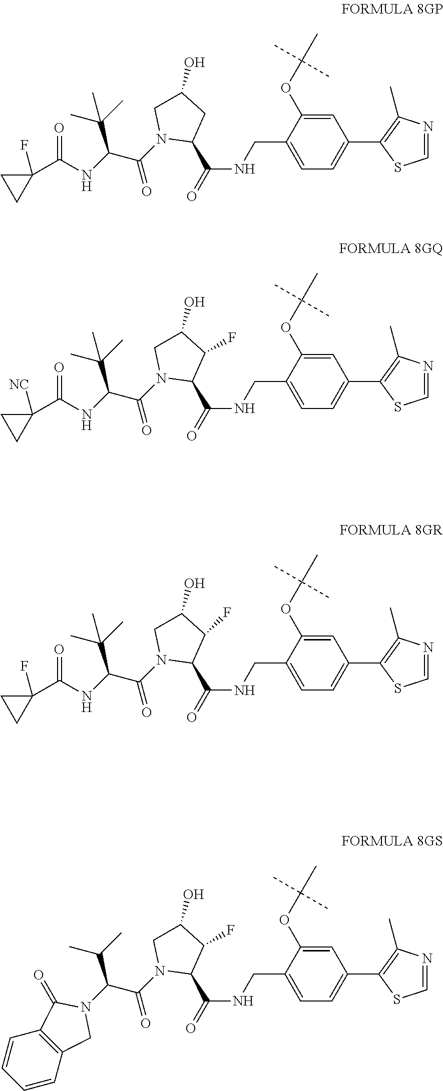 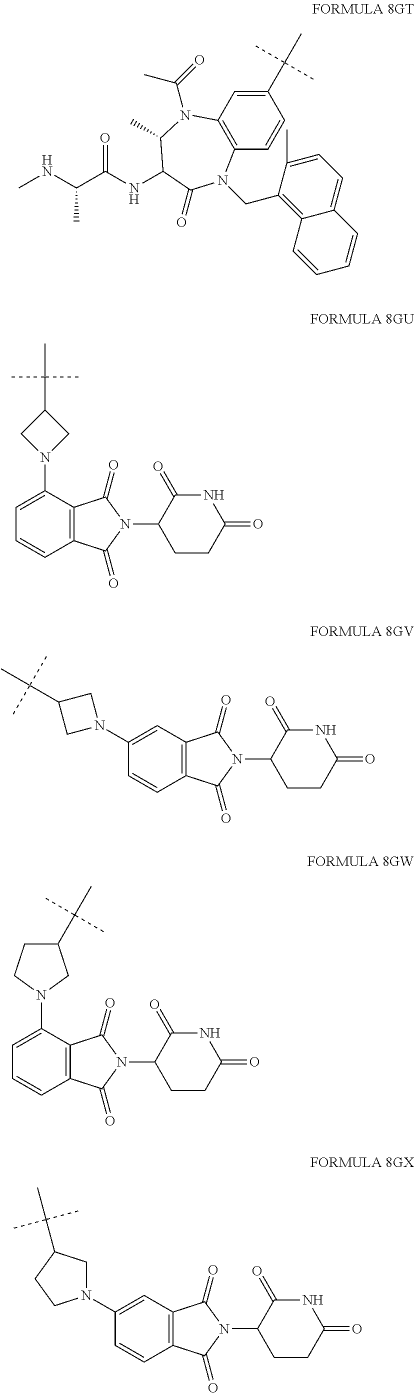 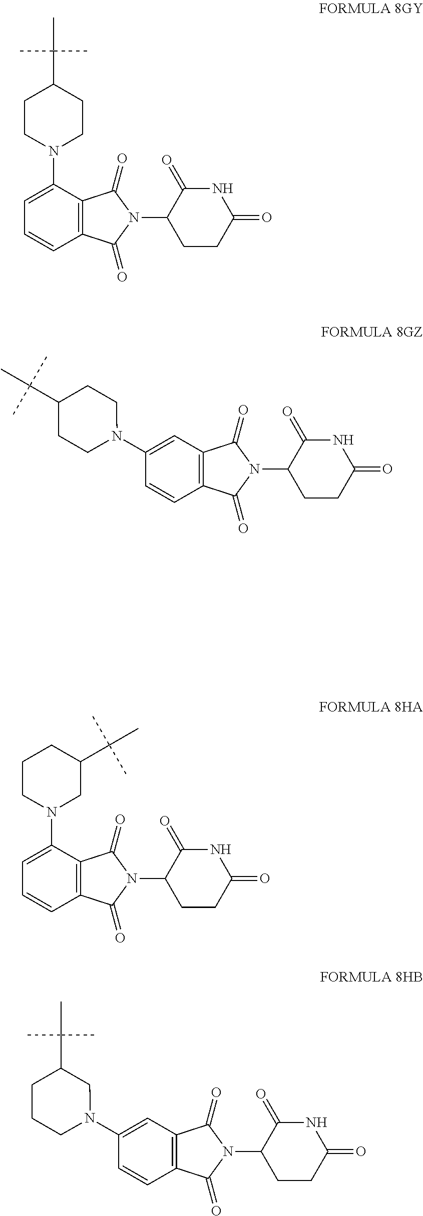 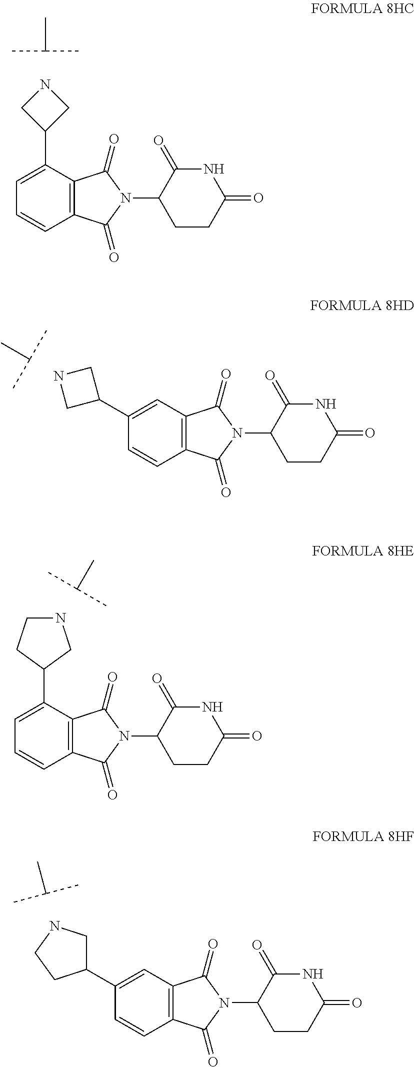 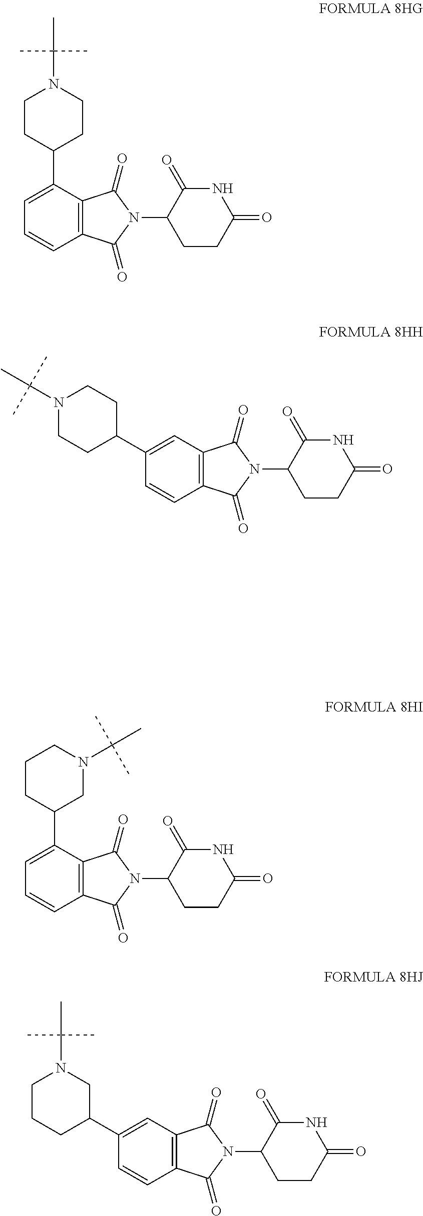 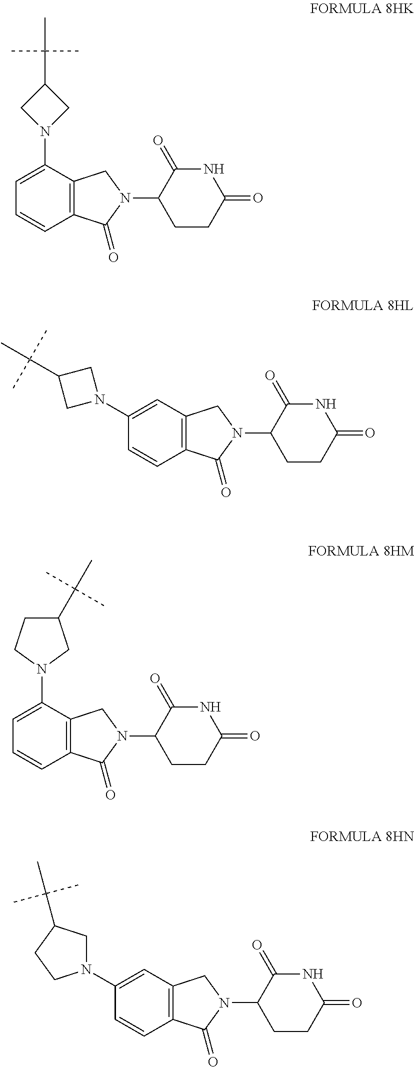 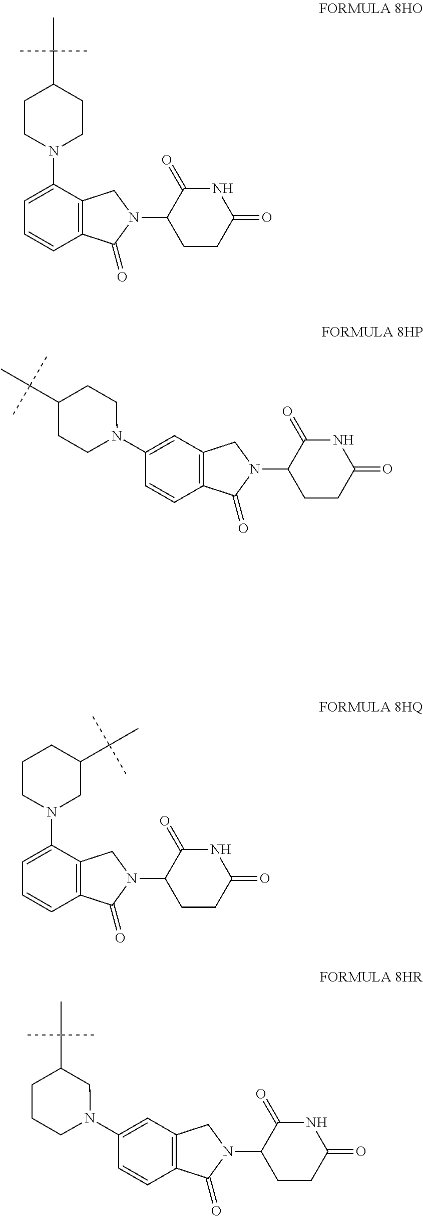 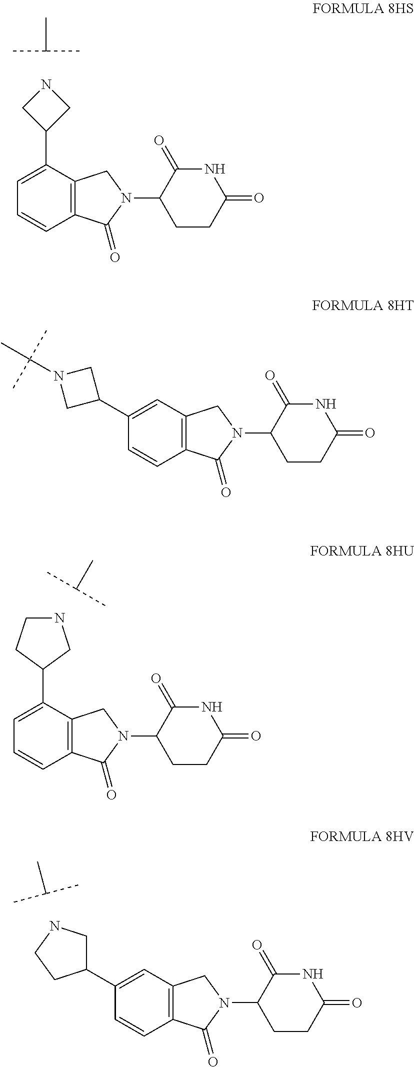 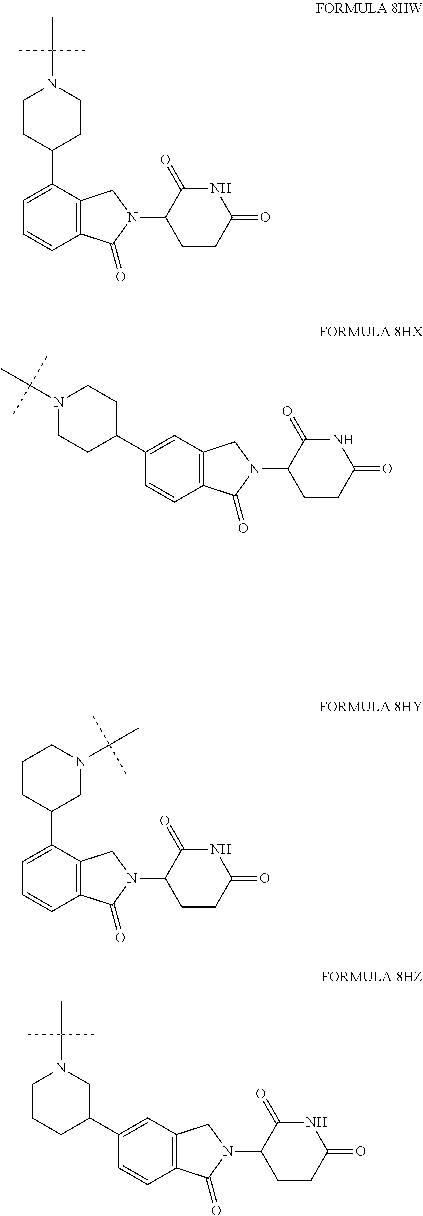 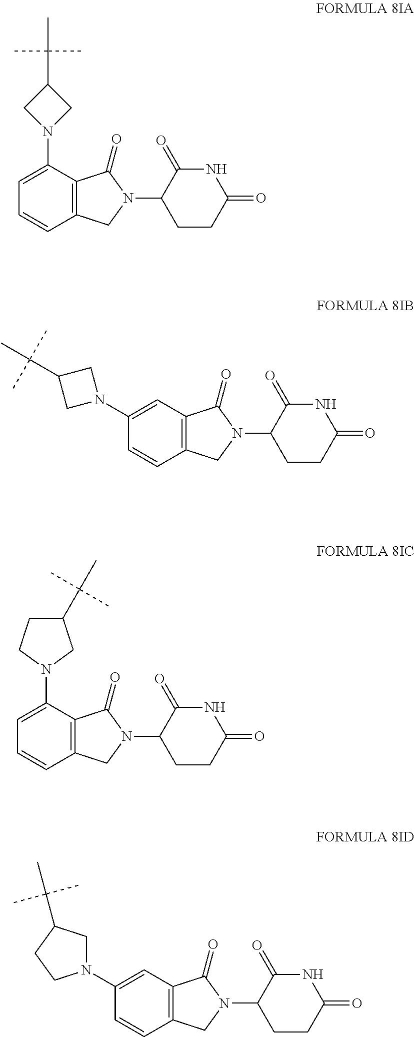 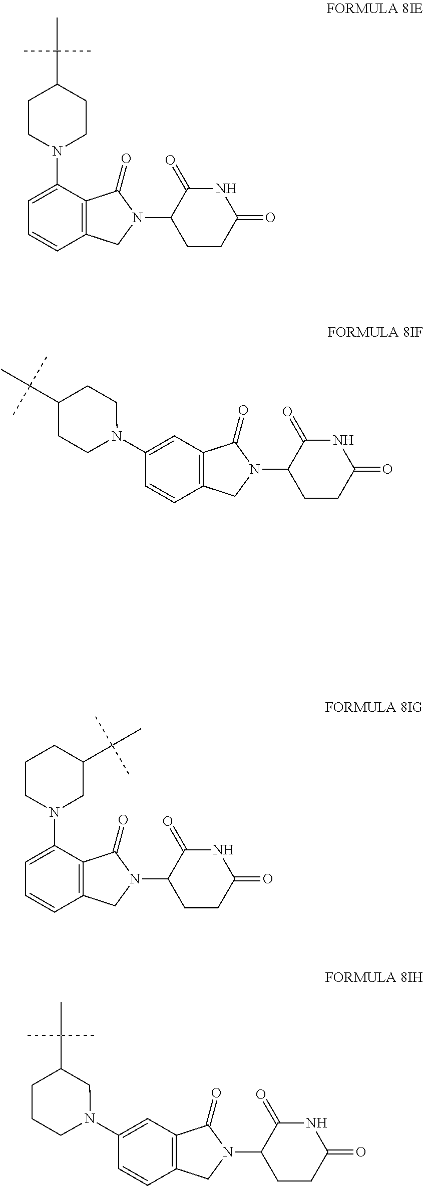 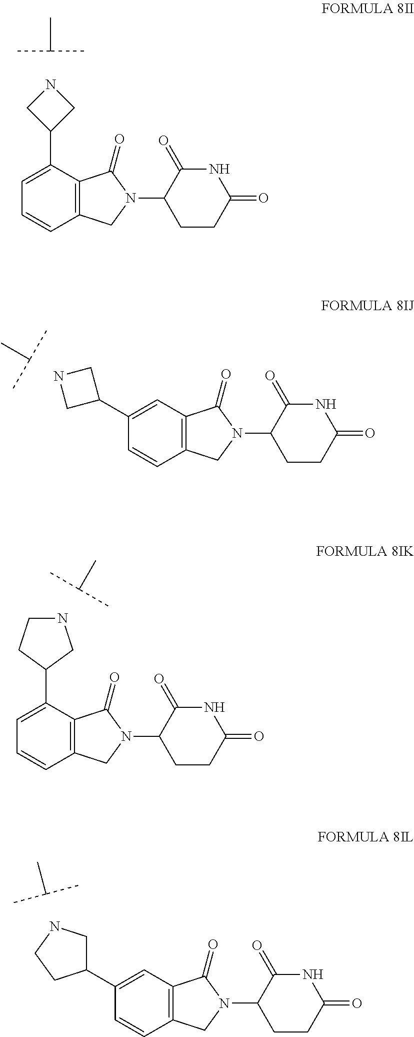 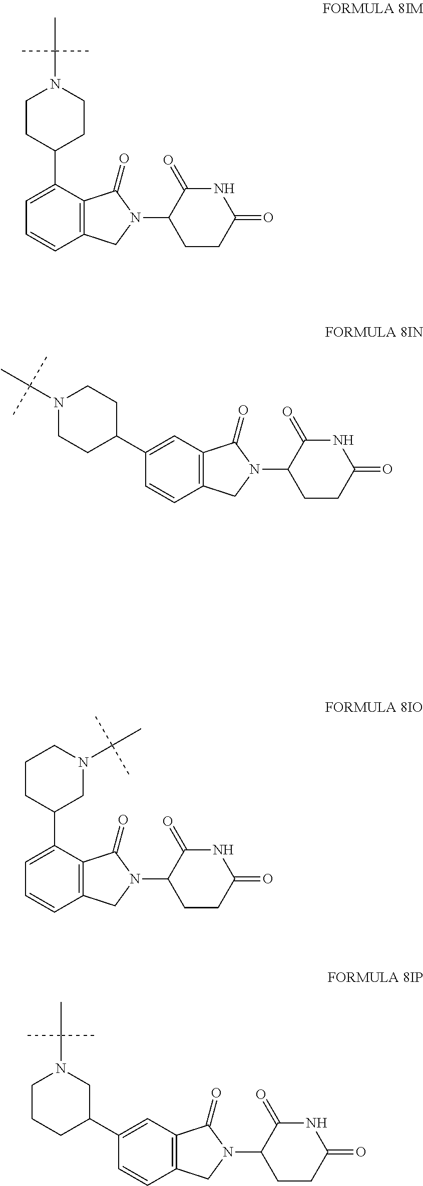 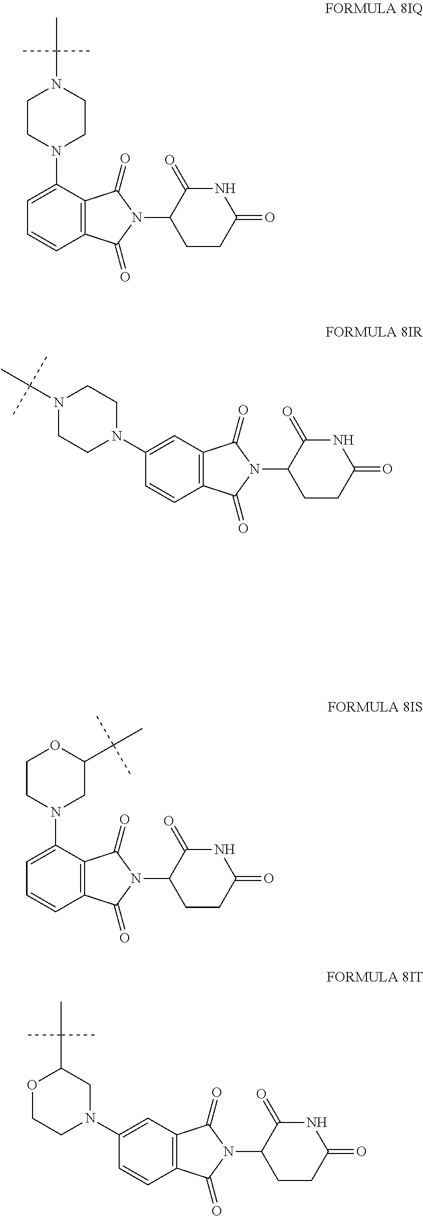 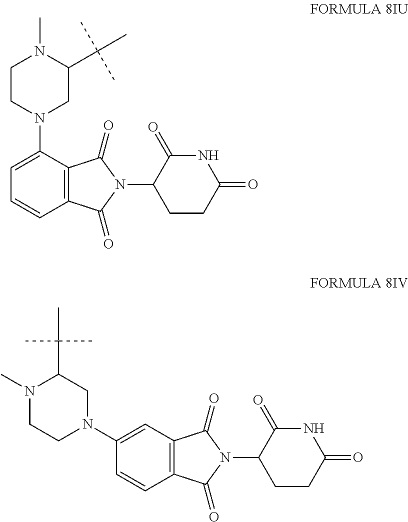 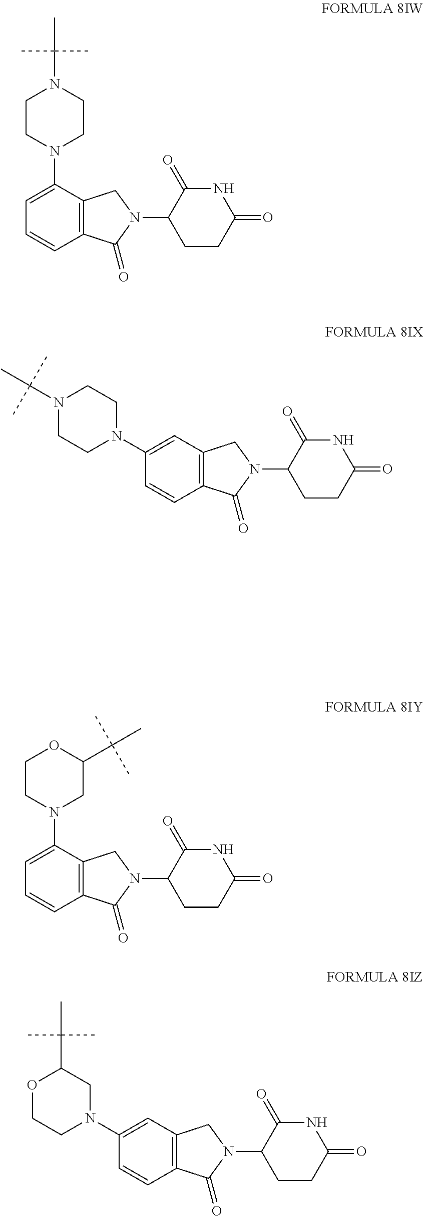 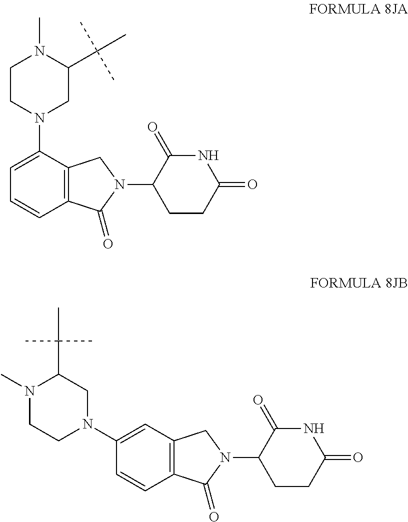 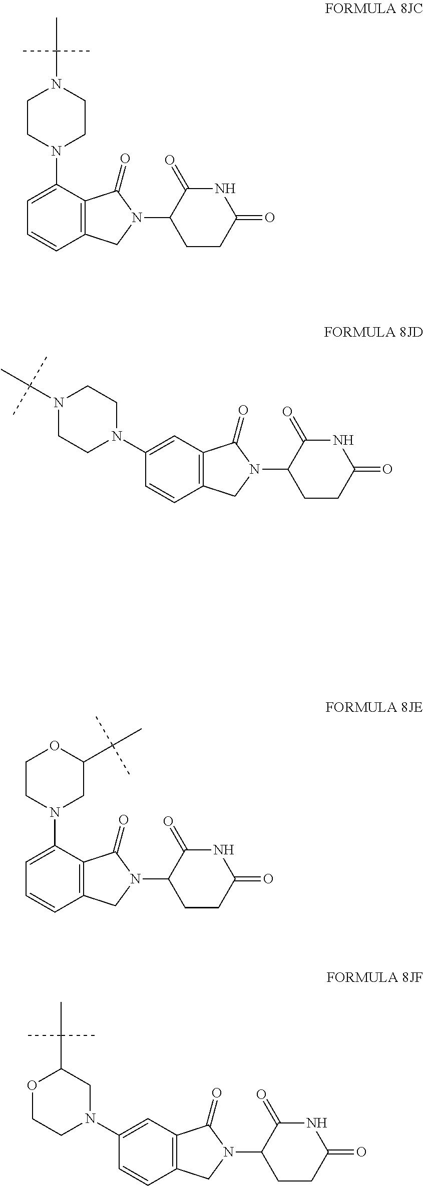 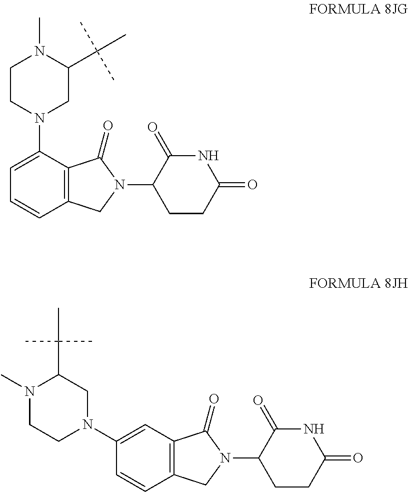 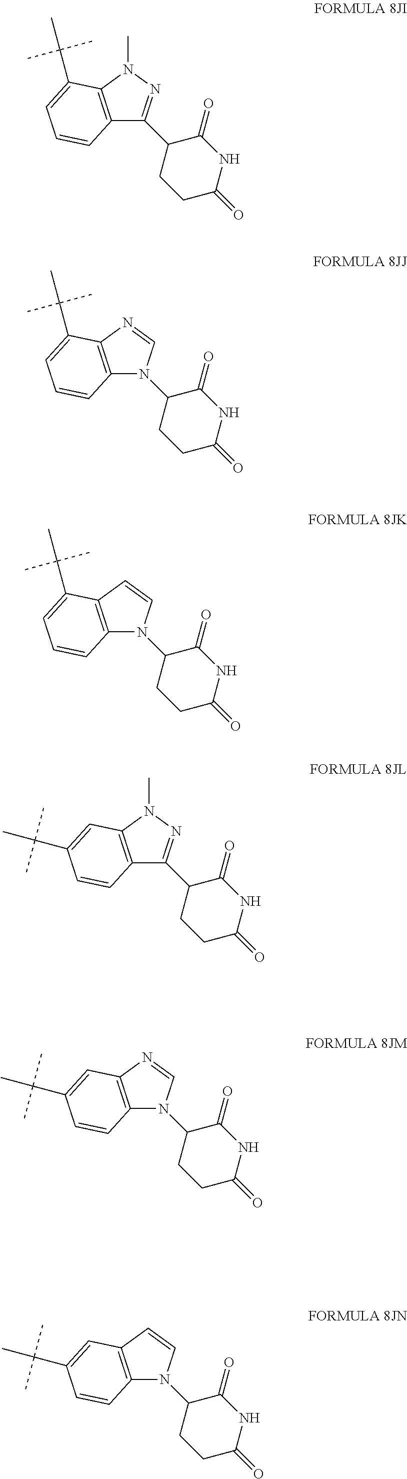 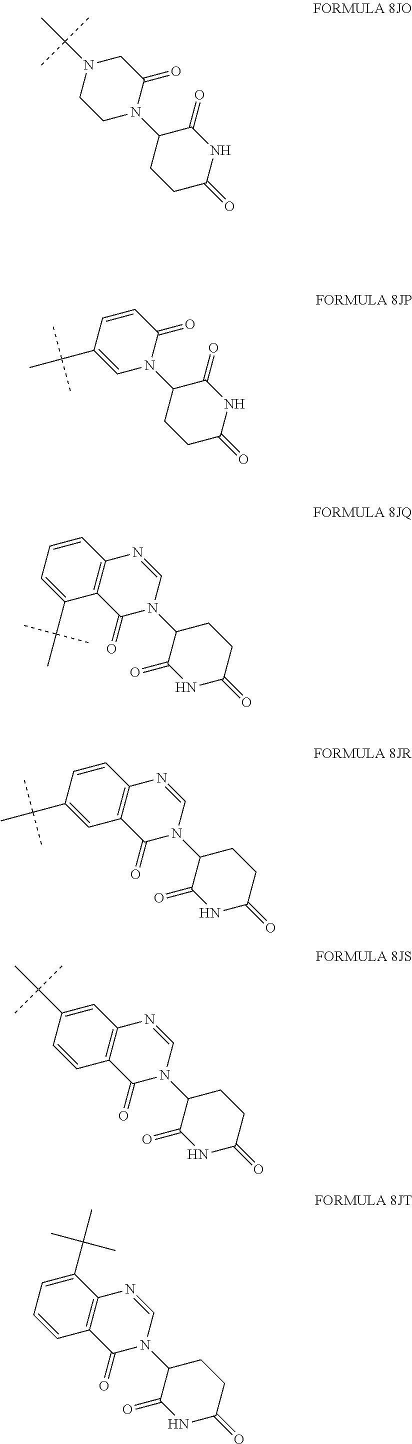
wherein the bond 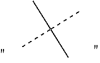
indicates the connection to the linker moiety of the bivalent compound.

48-55. (canceled)

56. The bivalent compound of claim 13, wherein
in FORMULAE 9, 9A, 9B, and 9C,
A, B, and W, at each occurrence, are independently selected from null, CO, NH, NH—CO, CO—NH, —(CH₂)₀₋₈—, —(CH₂)₀₋₃—CO—(CH₂)₀₋₈—, (CH₂)₀₋₈—NH—CO, (CH₂)₀₋₈—CO—NH, NH—CO—(CH₂)₀₋₈, CO—NH—(CH₂)₀₋₈, (CH₂)₁₋₃—NH—(CH₂)₁₋₃—CO—NH, (CH₂)₁₋₃—NH—(CH₂)₁₋₃—NH—CO, —CO—NH, CO—NH—(CH₂)₁₋₃—NH—(CH₂)₁₋₃, (CH₂)₁₋₃—NH—(CH₂)₁₋₃, —(CH₂)₀₋₃—R^{r}—(CH₂)₀₋₃, —(CH₂)₀₋₃—(CO)—(CH₂)₀₋₃—R^{r}—(CH₂)₀₋₃—, —(CH₂)₀₋₃—(CO—NH)—(CH₂)₀₋₃—R^{r}—(CH₂)₀₋₃—, —(CH₂)₀₋₃—(NH—CO)—(CH₂)₀₋₃—R^{r}—(CH₂)₀₋₃—, and —(CH₂)₀₋₃—(NH)—(CH₂)₀₋₃—R^{r}—(CH₂)₀₋₃—.

57. The bivalent compound of claim 13, wherein
in FORMULA 9, 9A, 9B or 9C,
A and B, at each occurrence, are independently selected from null, CO, NH, NH—CO, CO—NH, CH₂—NH—CO, CH₂—CO—NH, NH—CO—CH₂, CO—NH—CH₂, CH₂—NH—CH₂—CO—NH, CH₂—NH—CH₂—NH—CO, —CO—NH, CO—NH— CH₂—NH—CH₂, CH₂—NH—CH₂.

58. The bivalent compound of claim 13, wherein the linker moiety comprises one or more rings selected from the group consisting of FORMULA C1a, C2a, C3a, C4a and C5a: 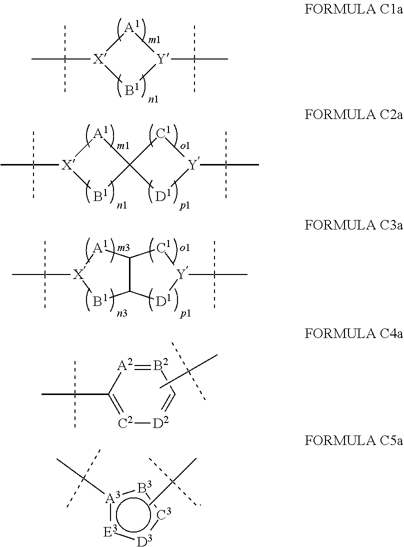
wherein
X′ and Y′ are independently selected from N, CR^{b};
A¹, B¹, C¹ and D¹, at each occurrence, are independently selected from null, O, CO, SO, SO₂, NR^{b}, CR^{b}R^{c};
A², B², C², and D², at each occurrence, are independently selected from N, CR^{b};
A³, B³, C³, D³, and E³, at each occurrence, are independently selected from N, O, S, NR^{b}, CR^{b};
R^{b} and R^{c}, at each occurrence, are independently selected from hydrogen, halogen, hydroxyl, amino, cyano, nitro, optionally substituted C₁-C₈ alkyl, optionally substituted C₂-C₈ alkenyl, optionally substituted C₂-C₈ alkynyl, optionally substituted C₁-C₈ alkoxy, optionally substituted C₁-C₈ alkoxyalkyl, optionally substituted C₁-C₈ haloalkyl, optionally substituted C₁-C₈ hydroxyalkyl, optionally substituted C₁-C₈ alkylamino, and optionally substituted C₁-C₈ alkylaminoC₁-C₈ alkyl, optionally substituted 3-10 membered carbocyclyl, optionally substituted 3-8 membered cycloalkoxy, optionally substituted 3-10 membered carbocyclylamino, optionally substituted 4-8 membered heterocyclyl, optionally substituted aryl, and optionally substituted heteroaryl; and
m¹, n¹, o¹ and p¹ are independently selected from 0, 1, 2, 3, 4 and 5.

59. (canceled)

60. The bivalent compound of claim 12, wherein the length of the linker moiety is 0 to 40 chain atoms; preferably the length of the linker is 3 to 20 chain atoms; more preferably the length of the linker is 5 to 15 chain atoms.

61. The bivalent compound of claim 12, wherein the linker moiety is selected from —(CH₂)₀₋₁₁—, —(CH₂)₀₋₃—CO—(CH₂)₀₋₁₀—, —(CO)—(CH₂)₃₋₁₀— (preferably, —(CO)—(CH₂)₃₋₇—), —(CH₂)₀₋₃(CONH)—(CH₂)₀₋₁₀— (preferably, —(CH₂)₀₋₃—(CONH)—(CH₂)₃₋₉, more preferably, —(CH₂)₁₋₂(CONH)—(CH₂)₃₋₇—), —(CH₂)₀₋₃—(CO)—(CH₂)₀₋₅—R^{r}—(CH₂)₀₋₅— (preferably, —(CH₂)₀₋₃—(CO)—(CH₂)₀₋₃—R^{r}—(CH₂)₀₋₃—), —(CH₂)₀₋₃—(CONH)—(CH₂)₀₋₅—R^{r}—(CH₂)₀₋₅— and —(CH₂)₀₋₅—R^{r}—(CH₂)₀₋₅, (preferably, —(CH₂)₀₋₃—R^{r}—(CH₂)₁₋₂).

62. The bivalent compound of claim 12, wherein the linker moiety is selected from —(CH₂)₀₋₁₁—, —(CH₂)₀₋₃—CO—(CH₂)₀₋₁₀—, —(CO)—(CH₂)₃₋₇—, —(CH₂)₀₋₃—(CONH)—(CH₂)₃₋₉, —(CH₂)₁₋₂(CO)—NH—(CH₂)₃₋₇—, —(CH₂)₀₋₃—(CO)—(CH₂)₀₋₃—Rᵣ—(CH₂)₀₋₃, and —(CH₂)₀₋₃—Rᵣ—(CH₂)₁₋₂.

63. (canceled)

64. The bivalent compound of claim 13, wherein
in FORMULA 9, 9A, 9B or 9C,
R^{r} selected from
FORMULA C1a, C2a, C3a, C4a, and C5a as defined in claim 58.

65. The bivalent compound of claim 13, wherein
in FORMULA 9, 9A, 9B or 9C,
R^{r} is selected from

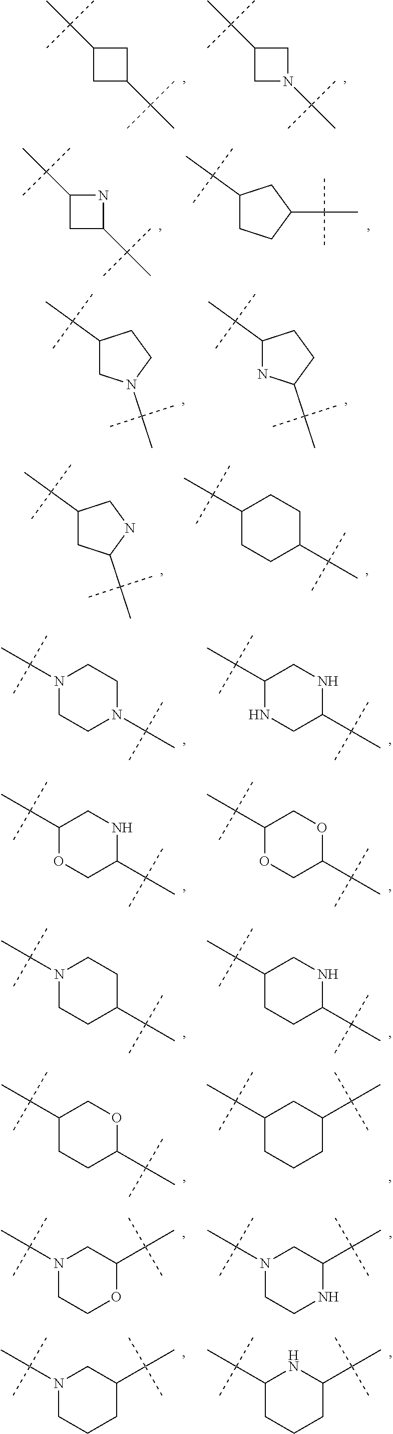

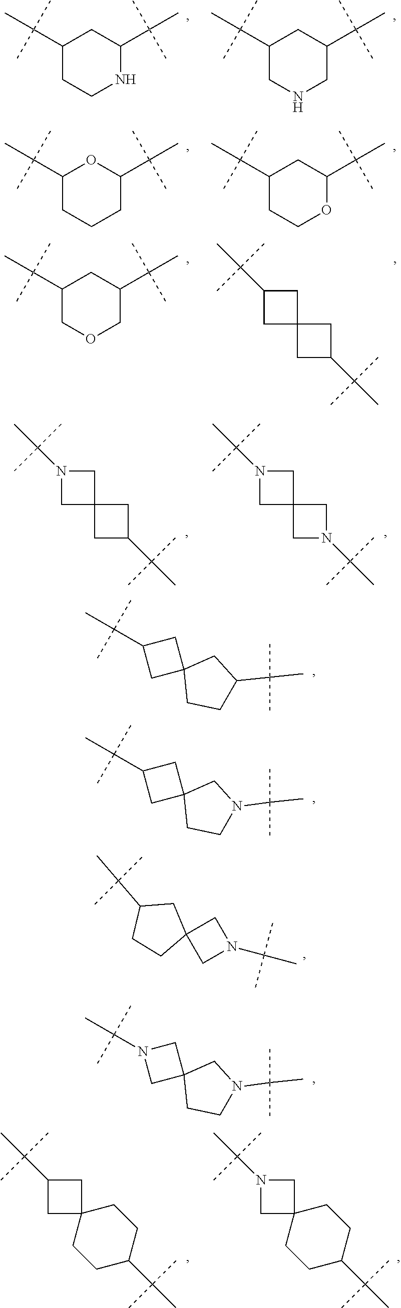

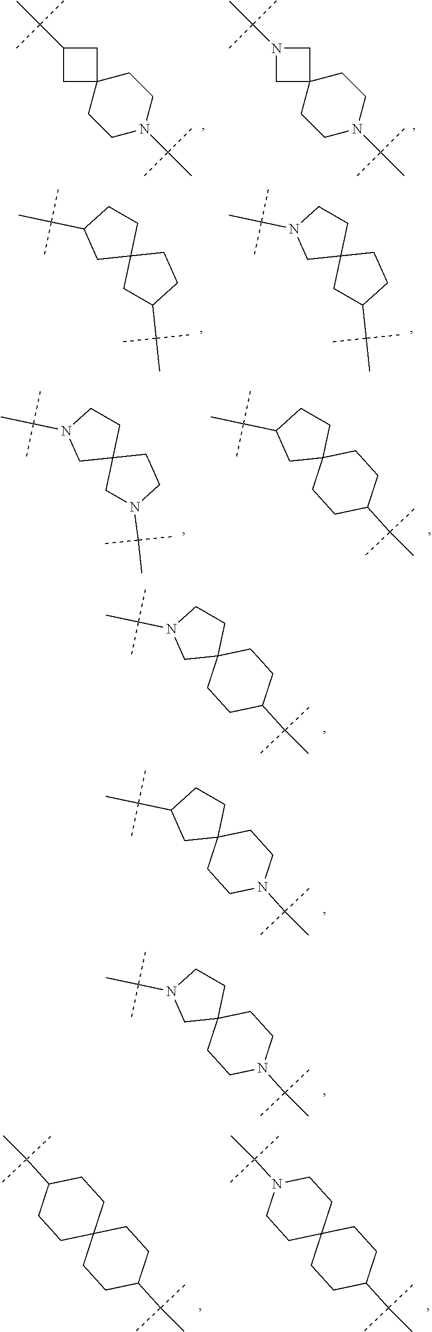

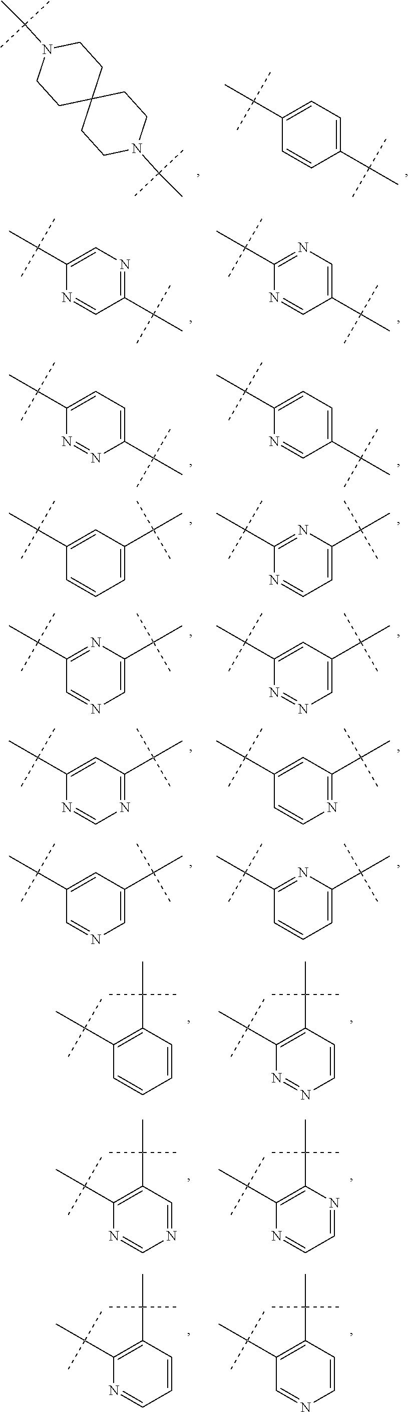

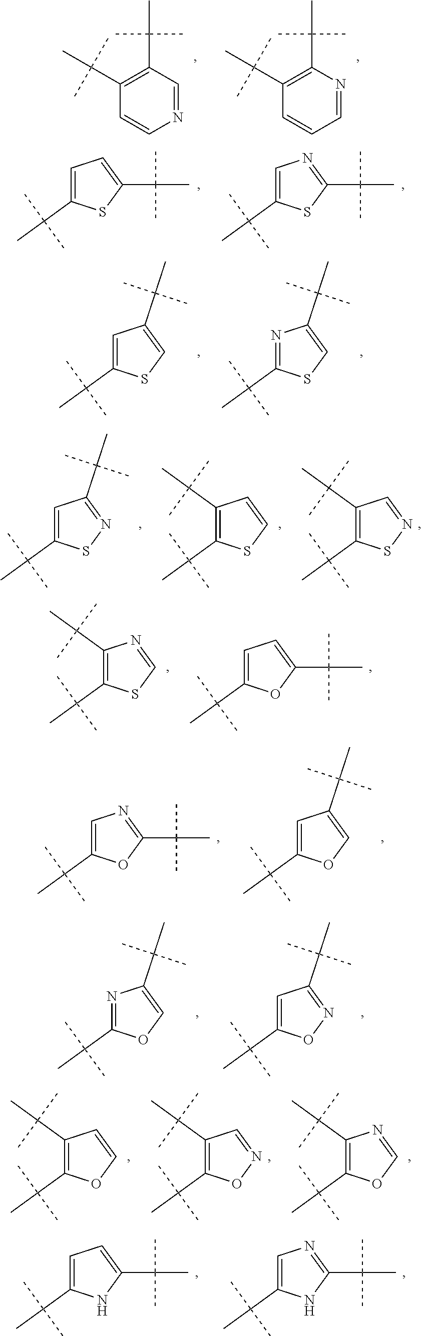

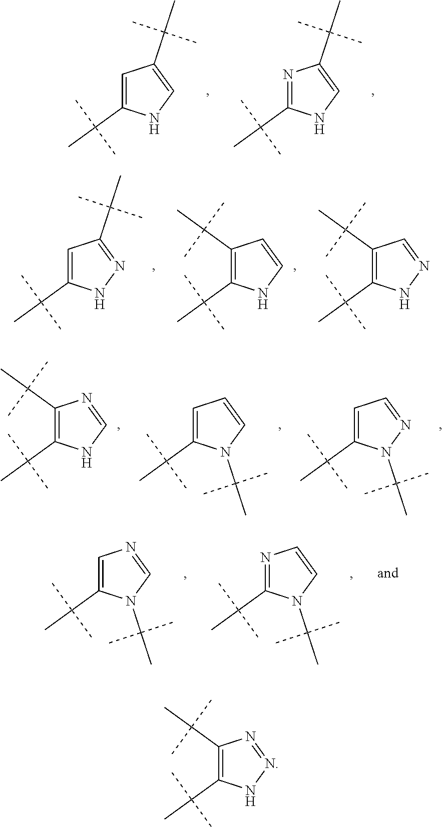

66. (canceled)

67. The bivalent compound of claim 12, wherein the bivalent compound is selected from the group consisting of P-001 to P-174 and CPD-1139 to CPD-1179 or a pharmaceutically acceptable salt or analog thereof, and a pharmaceutically acceptable carrier or diluent.

68. (canceled)

69. The bivalent compound of claim 12, wherein the bivalent compound is 3-(7-(Difluoromethyl)-6-(1-methyl-1H-pyrazol-4-yl)-3,4-dihydroquinolin-1(2H)-yl)-1-(1-(5-((2-(2,6-dioxopiperidin-3-yl)-1,3-dioxoisoindolin-5-yl)amino)pentanoyl)piperidin-4-yl)-N-methyl-1,4,6,7-tetrahydro-5H-pyrazolo[4,3-c]pyridine-5-carboxamide (P-004);
3-(7-(Difluoromethyl)-6-(1-methyl-1H-pyrazol-4-yl)-3,4-dihydroquinolin-1(2H)-yl)-1-(1-(6-((2-(2,6-dioxopiperidin-3-yl)-1,3-dioxoisoindolin-5-yl)amino)hexanoyl)piperidin-4-yl)-N-methyl-1,4,6,7-tetrahydro-5H-pyrazolo[4,3-c]pyridine-5-carboxamide (P-005);
3-(7-(Difluoromethyl)-6-(1-methyl-1H-pyrazol-4-yl)-3,4-dihydroquinolin-1(2H)-yl)-1-(1-(7-((2-(2,6-dioxopiperidin-3-yl)-1,3-dioxoisoindolin-5-yl)amino)heptanoyl)piperidin-4-yl)-N-methyl-1,4,6,7-tetrahydro-5H-pyrazolo[4,3-c]pyridine-5-carboxamide (P-006);
3-(7-(Difluoromethyl)-6-(1-methyl-1H-pyrazol-4-yl)-3,4-dihydroquinolin-1(2H)-yl)-1-(1-(8-((2-(2,6-dioxopiperidin-3-yl)-1,3-dioxoisoindolin-5-yl)amino)octanoyl)piperidin-4-yl)-N-methyl-1,4,6,7-tetrahydro-5H-pyrazolo[4,3-c]pyridine-5-carboxamide (P-007);
3-(7-(Difluoromethyl)-6-(1-methyl-1H-pyrazol-4-yl)-3,4-dihydroquinolin-1(2H)-yl)-1-(1-(4-((2-(2,6-dioxopiperidin-3-yl)-1,3-dioxoisoindolin-4-yl)amino)butanoyl)piperidin-4-yl)-N-methyl-1,4,6,7-tetrahydro-5H-pyrazolo[4,3-c]pyridine-5-carboxamide (P-015);
3-(7-(Difluoromethyl)-6-(1-(2-((6-((2-(2,6-dioxopiperidin-3-yl)-1,3-dioxoisoindolin-5-yl)amino)hexyl)amino)-2-oxoethyl)-1H-pyrazol-4-yl)-3,4-dihydroquinolin-1(2H)-yl)-N-methyl-1-(tetrahydro-2H-pyran-4-yl)-1,4,6,7-tetrahydro-5H-pyrazolo[4,3-c]pyridine-5-carboxamide (P-020),
3-(7-(Difluoromethyl)-6-(1-(2-((7-((2-(2,6-dioxopiperidin-3-yl)-1,3-dioxoisoindolin-5-yl)amino)heptyl)amino)-2-oxoethyl)-1H-pyrazol-4-yl)-3,4-dihydroquinolin-1(2H)-yl)-N-methyl-1-(tetrahydro-2H-pyran-4-yl)-1,4,6,7-tetrahydro-5H-pyrazolo[4,3-c]pyridine-5-carboxamide (P-026);
3-(7-(Difluoromethyl)-6-(1-(2-((8-((2-(2,6-dioxopiperidin-3-yl)-1,3-dioxoisoindolin-5-yl)amino)octyl)amino)-2-oxoethyl)-1H-pyrazol-4-yl)-3,4-dihydroquinolin-1(2H)-yl)-N-methyl-1-(tetrahydro-2H-pyran-4-yl)-1,4,6,7-tetrahydro-5H-pyrazolo[4,3-c]pyridine-5-carboxamide (P-027);
3-(7-(Difluoromethyl)-6-(1-methyl-1H-pyrazol-4-yl)-3,4-dihydroquinolin-1(2H)-yl)-1-(1-(5-((2-(2,6-dioxopiperidin-3-yl)-1,3-dioxoisoindolin-4-yl)amino)pentanoyl)piperidin-4-yl)-N-methyl-1,4,6,7-tetrahydro-5H-pyrazolo[4,3-c]pyridine-5-carboxamide (P-033);
3-(7-(Difluoromethyl)-6-(1-methyl-1H-pyrazol-4-yl)-3,4-dihydroquinolin-1(2H)-yl)-1-(1-(6-((2-(2,6-dioxopiperidin-3-yl)-1,3-dioxoisoindolin-4-yl)amino)hexanoyl)piperidin-4-yl)-N-methyl-1,4,6,7-tetrahydro-5H-pyrazolo[4,3-c]pyridine-5-carboxamide (P-034);
3-(7-(Difluoromethyl)-6-(1-methyl-1H-pyrazol-4-yl)-3,4-dihydroquinolin-1(2H)-yl)-1-(1-(7-((2-(2,6-dioxopiperidin-3-yl)-1,3-dioxoisoindolin-4-yl)amino)heptanoyl)piperidin-4-yl)-N-methyl-1,4,6,7-tetrahydro-5H-pyrazolo[4,3-c]pyridine-5-carboxamide (P-035);
3-(7-(Difluoromethyl)-6-(1-methyl-1H-pyrazol-4-yl)-3,4-dihydroquinolin-1(2H)-yl)-1-(1-(8-((2-(2,6-dioxopiperidin-3-yl)-1,3-dioxoisoindolin-4-yl)amino)octanoyl)piperidin-4-yl)-N-methyl-1,4,6,7-tetrahydro-5H-pyrazolo[4,3-c]pyridine-5-carboxamide (P-036); or
3-(7-(Difluoromethyl)-6-(1-(2-((6-((2-(2,6-dioxopiperidin-3-yl)-1,3-dioxoisoindolin-4-yl)amino)hexyl)amino)-2-oxoethyl)-1H-pyrazol-4-yl)-3,4-dihydroquinolin-1(2H)-yl)-N-methyl-1-(tetrahydro-2H-pyran-4-yl)-1,4,6,7-tetrahydro-5H-pyrazolo[4,3-c]pyridine-5-carboxamide (P-041);
3-(7-(difluoromethyl)-6-(1-(2-((8-((2-(2,6-dioxopiperidin-3-yl)-1,3-dioxoisoindolin-4-yl)amino)octyl)amino)-2-oxoethyl)-1H-pyrazol-4-yl)-3,4-dihydroquinolin-1(2H)-yl)-N-methyl-1-(tetrahydro-2H-pyran-4-yl)-1,4,6,7-tetrahydro-5H-pyrazolo[4,3-c]pyridine-5-carboxamide (P-043);
3-(7-(difluoromethyl)-6-(1-methyl-1H-pyrazol-4-yl)-3,4-dihydroquinolin-1(2H)-yl)-1-(1-(7-(2-(2,6-dioxopiperidin-3-yl)-1,3-dioxoisoindolin-4-yl)heptanoyl)piperidin-4-yl)-N-methyl-1,4,6,7-tetrahydro-5H-pyrazolo[4,3-c]pyridine-5-carboxamide (P-085);
3-(7-(difluoromethyl)-6-(1-methyl-1H-pyrazol-4-yl)-3,4-dihydroquinolin-1(2H)-yl)-1-(1-(6-((2-(2,6-dioxopiperidin-3-yl)-1-oxoisoindolin-4-yl)oxy)hexanoyl)piperidin-4-yl)-N-methyl-1,4,6,7-tetrahydro-5H-pyrazolo[4,3-c]pyridine-5-carboxamide (P-088);
3-(7-(difluoromethyl)-6-(1-methyl-1H-pyrazol-4-yl)-3,4-dihydroquinolin-1(2H)-yl)-1-(1-(6-((2-(2,6-dioxopiperidin-3-yl)-1,3-dioxoisoindolin-4-yl)amino)hexyl)piperidin-4-yl)-N-methyl-1,4,6,7-tetrahydro-5H-pyrazolo[4,3-c]pyridine-5-carboxamide (P-090);
3-(7-(difluoromethyl)-6-(1-methyl-1H-pyrazol-4-yl)-3,4-dihydroquinolin-1(2H)-yl)-1-(1-(6-((2-(2,6-dioxopiperidin-3-yl)-1,3-dioxoisoindolin-4-yl)oxy)hexyl)piperidin-4-yl)-N-methyl-1,4,6,7-tetrahydro-5H-pyrazolo[4,3-c]pyridine-5-carboxamide (P-091);
3-(7-(difluoromethyl)-6-(1-methyl-1H-pyrazol-4-yl)-3,4-dihydroquinolin-1(2H)-yl)-1-(1-(7-(2-(2,6-dioxopiperidin-3-yl)-1-oxoisoindolin-4-yl)hept-6-ynoyl)piperidin-4-yl)-N-methyl-1,4,6,7-tetrahydro-5H-pyrazolo[4,3-c]pyridine-5-carboxamide (P-093);
3-(7-(difluoromethyl)-6-(1-methyl-1H-pyrazol-4-yl)-3,4-dihydroquinolin-1(2H)-yl)-1-(1-(6-((2-(2,6-dioxopiperidin-3-yl)-3-oxoisoindolin-4-yl)amino)hexanoyl)piperidin-4-yl)-N-methyl-1,4,6,7-tetrahydro-5H-pyrazolo[4,3-c]pyridine-5-carboxamide (P-096);
3-(7-(difluoromethyl)-6-(1-methyl-1H-pyrazol-4-yl)-3,4-dihydroquinolin-1(2H)-yl)-1-(1-(7-(2-(2,6-dioxopiperidin-3-yl)-1,3-dioxoisoindolin-4-yl)heptyl)piperidin-4-yl)-N-methyl-1,4,6,7-tetrahydro-5H-pyrazolo[4,3-c]pyridine-5-carboxamide (P-097);
4-((6-(4-(5-acetyl-3-(7-(difluoromethyl)-6-(1-methyl-1H-pyrazol-4-yl)-3,4-dihydroquinolin-1(2H)-yl)-4,5,6,7-tetrahydro-1H-pyrazolo[4,3-c]pyridin-1-yl)piperidin-1-yl)-6-oxohexyl)amino)-2-(2,6-dioxopiperidin-3-yl)isoindoline-1,3-dione (P-100);
3-(7-(difluoromethyl)-6-(1-methyl-1H-pyrazol-4-yl)-3,4-dihydroquinolin-1(2H)-yl)-1-(1-(2-(4-(2-(2-(2,6-dioxopiperidin-3-yl)-1,3-dioxoisoindolin-4-yl)ethyl)piperidin-1-yl)acetyl)piperidin-4-yl)-N-methyl-1,4,6,7-tetrahydro-5H-pyrazolo[4,3-c]pyridine-5-carboxamide (P-104);
3-(7-(difluoromethyl)-6-(1-methyl-1H-pyrazol-4-yl)-3,4-dihydroquinolin-1(2H)-yl)-1-(1-(7-(3-(2,6-dioxopiperidin-3-yl)-2-oxo-2,3-dihydro-1H-benzo[d]imidazol-1-yl)heptanoyl)piperidin-4-yl)-N-methyl-1,4,6,7-tetrahydro-5H-pyrazolo[4,3-c]pyridine-5-carboxamide (P-106);
3-(7-(difluoromethyl)-6-(1-methyl-1H-pyrazol-4-yl)-3,4-dihydroquinolin-1(2H)-yl)-1-(1-(6-((1-(2,6-dioxopiperidin-3-yl)-3-methyl-2-oxo-2,3-dihydro-1H-benzo[d]imidazol-4-yl)amino)hexanoyl)piperidin-4-yl)-N-methyl-1,4,6,7-tetrahydro-5H-pyrazolo[4,3-c]pyridine-5-carboxamide (P-109);
3-(4-((6-(4-(5-acetyl-3-(7-(difluoromethyl)-6-(1-methyl-1H-pyrazol-4-yl)-3,4-dihydroquinolin-1(2H)-yl)-4,5,6,7-tetrahydro-1H-pyrazolo[4,3-c]pyridin-1-yl)piperidin-1-yl)-6-oxohexyl)amino)-3-methyl-2-oxo-2,3-dihydro-1H-benzo[d]imidazol-1-yl)piperidine-2,6-dione (P-110);
3-(4-((7-(4-(5-acetyl-3-(7-(difluoromethyl)-6-(1-methyl-1H-pyrazol-4-yl)-3,4-dihydroquinolin-1(2H)-yl)-4,5,6,7-tetrahydro-1H-pyrazolo[4,3-c]pyridin-1-yl)piperidin-1-yl)-7-oxoheptyl)amino)-3-methyl-2-oxo-2,3-dihydro-1H-benzo[d]imidazol-1-yl)piperidine-2,6-dione (P-111);
4-((6-(4-(5-acetyl-3-(7-(difluoromethyl)-6-(1-methyl-1H-pyrazol-4-yl)-3,4-dihydroquinolin-1(2H)-yl)-4,5,6,7-tetrahydro-1H-pyrazolo[4,3-c]pyridin-1-yl)piperidin-1-yl)hexyl)amino)-2-(2,6-dioxopiperidin-3-yl)isoindoline-1,3-dione (P-112);
4-((6-(4-(5-acetyl-3-(7-(difluoromethyl)-6-(1-methyl-1H-pyrazol-4-yl)-3,4-dihydroquinolin-1(2H)-yl)-4,5,6,7-tetrahydro-1H-pyrazolo[4,3-c]pyridin-1-yl)piperidin-1-yl)hexyl)oxy)-2-(2,6-dioxopiperidin-3-yl)isoindoline-1,3-dione (P-113);
3-(7-(difluoromethyl)-6-(1-methyl-1H-pyrazol-4-yl)-3,4-dihydroquinolin-1(2H)-yl)-1-(1-(6-((1-(2,6-dioxopiperidin-3-yl)-3-methyl-2-oxo-2,3-dihydro-1H-benzo[d]imidazol-5-yl)amino)hexanoyl)piperidin-4-yl)-N-methyl-1,4,6,7-tetrahydro-5H-pyrazolo[4,3-c]pyridine-5-carboxamide (P-115);
3-(5-((6-(4-(5-acetyl-3-(7-(difluoromethyl)-6-(1-methyl-1H-pyrazol-4-yl)-3,4-dihydroquinolin-1(2H)-yl)-4,5,6,7-tetrahydro-1H-pyrazolo[4,3-c]pyridin-1-yl)piperidin-1-yl)-6-oxohexyl)amino)-3-methyl-2-oxo-2,3-dihydro-1H-benzo[d]imidazol-1-yl)piperidine-2,6-dione (P-116);
5-((8-(4-(5-acetyl-3-(7-(difluoromethyl)-6-(1-methyl-1H-pyrazol-4-yl)-3,4-dihydroquinolin-1(2H)-yl)-4,5,6,7-tetrahydro-1H-pyrazolo[4,3-c]pyridin-1-yl)piperidin-1-yl)-8-oxooctyl)amino)-2-(2,6-dioxopiperidin-3-yl)isoindoline-1,3-dione (P-119);
5-((6-(4-(5-acetyl-3-(7-(difluoromethyl)-6-(1-methyl-1H-pyrazol-4-yl)-3,4-dihydroquinolin-1(2H)-yl)-4,5,6,7-tetrahydro-1H-pyrazolo[4,3-c]pyridin-1-yl)piperidin-1-yl)-6-oxohexyl)amino)-2-(2,6-dioxopiperidin-3-yl)isoindoline-1,3-dione (P-120);
3-(7-(difluoromethyl)-6-(1-methyl-1H-pyrazol-4-yl)-3,4-dihydroquinolin-1(2H)-yl)-1-(1-(7-((1-(2,6-dioxopiperidin-3-yl)-3-methyl-2-oxo-2,3-dihydro-1H-benzo[d]imidazol-5-yl)amino)heptanoyl)piperidin-4-yl)-N-methyl-1,4,6,7-tetrahydro-5H-pyrazolo[4,3-c]pyridine-5-carboxamide (P-129);
3-(5-((7-(4-(5-acetyl-3-(7-(difluoromethyl)-6-(1-methyl-1H-pyrazol-4-yl)-3,4-dihydroquinolin-1(2H)-yl)-4,5,6,7-tetrahydro-1H-pyrazolo[4,3-c]pyridin-1-yl)piperidin-1-yl)-7-oxoheptyl)amino)-3-methyl-2-oxo-2,3-dihydro-1H-benzo[d]imidazol-1-yl)piperidine-2,6-dione (P-130);
4-(2-(1-(2-(4-(5-acetyl-3-(7-(difluoromethyl)-6-(1-methyl-1H-pyrazol-4-yl)-3,4-dihydroquinolin-1(2H)-yl)-4,5,6,7-tetrahydro-1H-pyrazolo[4,3-c]pyridin-1-yl)piperidin-1-yl)-2-oxoethyl)piperidin-4-yl)ethyl)-2-(2,6-dioxopiperidin-3-yl)isoindoline-1,3-dione (P-131);
2-(4-(1-(5-acetyl-1-(tetrahydro-2H-pyran-4-yl)-4,5,6,7-tetrahydro-1H-pyrazolo[4,3-c]pyridin-3-yl)-7-(difluoromethyl)-1,2,3,4-tetrahydroquinolin-6-yl)-1H-pyrazol-1-yl)-N-(7-((2-(2,6-dioxopiperidin-3-yl)-1,3-dioxoisoindolin-5-yl)amino)heptyl)acetamide (P-133);
3-(3-(8-(4-(5-Acetyl-3-(7-(difluoromethyl)-6-(1-methyl-1H-pyrazol-4-yl)-3,4-dihydroquinolin-1(2H)-yl)-4,5,6,7-tetrahydro-1H-pyrazolo[4,3-c]pyridin-1-yl)piperidin-1-yl)-8-oxooctyl)-2-oxo-2,3-dihydro-1H-benzo[d]imidazol-1-yl)piperidine-2,6-dione (P-135);
4-(((4-(2-(4-(5-acetyl-3-(7-(difluoromethyl)-6-(1-methyl-1H-pyrazol-4-yl)-3,4-dihydroquinolin-1(2H)-yl)-4,5,6,7-tetrahydro-1H-pyrazolo[4,3-c]pyridin-1-yl)piperidin-1-yl)-2-oxoethyl)morpholin-2-yl)methyl)amino)-2-(2,6-dioxopiperidin-3-yl)isoindoline-1,3-dione (P-142);
4-(((1-(2-(4-(5-acetyl-3-(7-(difluoromethyl)-6-(1-methyl-1H-pyrazol-4-yl)-3,4-dihydroquinolin-1(2H)-yl)-4,5,6,7-tetrahydro-1H-pyrazolo[4,3-c]pyridin-1-yl)piperidin-1-yl)ethyl)piperidin-4-yl)methyl)amino)-2-(2,6-dioxopiperidin-3-yl)isoindoline-1,3-dione (P-143);
4-(3-(4-(4-(5-acetyl-3-(7-(difluoromethyl)-6-(1-methyl-1H-pyrazol-4-yl)-3,4-dihydroquinolin-1(2H)-yl)-4,5,6,7-tetrahydro-1H-pyrazolo[4,3-c]pyridin-1-yl)piperidin-1-yl)-4-oxobutyl)azetidin-1-yl)-2-(2,6-dioxopiperidin-3-yl)isoindoline-1,3-dione (P-146);
4-(3-(1-(2-(4-(5-acetyl-3-(7-(difluoromethyl)-6-(1-methyl-1H-pyrazol-4-yl)-3,4-dihydroquinolin-1(2H)-yl)-4,5,6,7-tetrahydro-1H-pyrazolo[4,3-c]pyridin-1-yl)piperidin-1-yl)-2-oxoethyl)piperidin-4-yl)propyl)-2-(2,6-dioxopiperidin-3-yl)isoindoline-1,3-dione (P-147);
4-(2-(1-(3-(4-(5-acetyl-3-(7-(difluoromethyl)-6-(1-methyl-1H-pyrazol-4-yl)-3,4-dihydroquinolin-1(2H)-yl)-4,5,6,7-tetrahydro-1H-pyrazolo[4,3-c]pyridin-1-yl)piperidin-1-yl)-3-oxopropyl)piperidin-4-yl)ethyl)-2-(2,6-dioxopiperidin-3-yl)isoindoline-1,3-dione (P-148);
4-(3-(4-((4-(5-acetyl-3-(7-(difluoromethyl)-6-(1-methyl-1H-pyrazol-4-yl)-3,4-dihydroquinolin-1(2H)-yl)-4,5,6,7-tetrahydro-1H-pyrazolo[4,3-c]pyridin-1-yl)piperidin-1-yl)methyl)piperidin-1-yl)propyl)-2-(2,6-dioxopiperidin-3-yl)isoindoline-1,3-dione (P-149);
3-(5-((6-(4-(5-acetyl-3-(7-(difluoromethyl)-6-(1-methyl-1H-pyrazol-4-yl)-3,4-dihydroquinolin-1(2H)-yl)-4,5,6,7-tetrahydro-1H-pyrazolo[4,3-c]pyridin-1-yl)piperidin-1-yl)-6-oxohexyl)amino)-4-oxobenzo[d][1,2,3]triazin-3 (4H)-yl)piperidine-2,6-dione (P-151);
3-(5-((7-(4-(5-acetyl-3-(7-(difluoromethyl)-6-(1-methyl-1H-pyrazol-4-yl)-3,4-dihydroquinolin-1(2H)-yl)-4,5,6,7-tetrahydro-1H-pyrazolo[4,3-c]pyridin-1-yl)piperidin-1-yl)-7-oxoheptyl)amino)-4-oxobenzo[d][1,2,3]triazin-3(4H)-yl)piperidine-2,6-dione (P-153);
3-(5-((5-(4-(5-acetyl-3-(7-(difluoromethyl)-6-(1-methyl-1H-pyrazol-4-yl)-3,4-dihydroquinolin-1(2H)-yl)-4,5,6,7-tetrahydro-1H-pyrazolo[4,3-c]pyridin-1-yl)piperidin-1-yl)-5-oxopentyl)amino)-4-oxobenzo[d][1,2,3]triazin-3(4H)-yl)piperidine-2,6-dione (P-155);
4-(2-(1-(2-(4-(5-acetyl-3-(7-(difluoromethyl)-6-(1-methyl-1H-pyrazol-4-yl)-3,4-dihydroquinolin-1(2H)-yl)-4,5,6,7-tetrahydro-1H-pyrazolo[4,3-c]pyridin-1-yl)piperidin-1-yl)ethyl)piperidin-4-yl)ethyl)-2-(2,6-dioxopiperidin-3-yl)isoindoline-1,3-dione (P-157);
3-(4-((8-(4-(5-acetyl-3-(7-(difluoromethyl)-6-(1-methyl-1H-pyrazol-4-yl)-3,4-dihydroquinolin-1(2H)-yl)-4,5,6,7-tetrahydro-1H-pyrazolo[4,3-c]pyridin-1-yl)piperidin-1-yl)-8-oxooctyl)amino)-3-methyl-2-oxo-2,3-dihydro-1H-benzo[d]imidazol-1-yl)piperidine-2,6-dione (P-159);
3-(4-((9-(4-(5-acetyl-3-(7-(difluoromethyl)-6-(1-methyl-1H-pyrazol-4-yl)-3,4-dihydroquinolin-1(2H)-yl)-4,5,6,7-tetrahydro-1H-pyrazolo[4,3-c]pyridin-1-yl)piperidin-1-yl)-9-oxononyl)amino)-3-methyl-2-oxo-2,3-dihydro-1H-benzo[d]imidazol-1-yl)piperidine-2,6-dione (P-160);
3-(5-((8-(4-(5-acetyl-3-(7-(difluoromethyl)-6-(1-methyl-1H-pyrazol-4-yl)-3,4-dihydroquinolin-1(2H)-yl)-4,5,6,7-tetrahydro-1H-pyrazolo[4,3-c]pyridin-1-yl)piperidin-1-yl)-8-oxooctyl)amino)-3-methyl-2-oxo-2,3-dihydro-1H-benzo[d]imidazol-1-yl)piperidine-2,6-dione (P-161);
3-(5-((9-(4-(5-acetyl-3-(7-(difluoromethyl)-6-(1-methyl-1H-pyrazol-4-yl)-3,4-dihydroquinolin-1(2H)-yl)-4,5,6,7-tetrahydro-1H-pyrazolo[4,3-c]pyridin-1-yl)piperidin-1-yl)-9-oxononyl)amino)-3-methyl-2-oxo-2,3-dihydro-1H-benzo[d]imidazol-1-yl)piperidine-2,6-dione (P-162);
3-(3-(9-(4-(5-acetyl-3-(7-(difluoromethyl)-6-(1-methyl-1H-pyrazol-4-yl)-3,4-dihydroquinolin-1(2H)-yl)-4,5,6,7-tetrahydro-1H-pyrazolo[4,3-c]pyridin-1-yl)piperidin-1-yl)-9-oxononyl)-2-oxo-2,3-dihydro-1H-benzo[d]imidazol-1-yl)piperidine-2,6-dione (P-163);
3-(3-(10-(4-(5-acetyl-3-(7-(difluoromethyl)-6-(1-methyl-1H-pyrazol-4-yl)-3,4-dihydroquinolin-1(2H)-yl)-4,5,6,7-tetrahydro-1H-pyrazolo[4,3-c]pyridin-1-yl)piperidin-1-yl)-10-oxodecyl)-2-oxo-2,3-dihydro-1H-benzo[d]imidazol-1-yl)piperidine-2,6-dione (P-164);
3-(4-((4-((4-(5-acetyl-3-(7-(difluoromethyl)-6-(1-methyl-1H-pyrazol-4-yl)-3,4-dihydroquinolin-1(2H)-yl)-4,5,6,7-tetrahydro-1H-pyrazolo[4,3-c]pyridin-1-yl)piperidin-1-yl)methyl)benzyl)oxy)-1-oxoisoindolin-2-yl)piperidine-2,6-dione (P-166);
3-(4-((4-(2-(4-(5-acetyl-3-(7-(difluoromethyl)-6-(1-methyl-1H-pyrazol-4-yl)-3,4-dihydroquinolin-1(2H)-yl)-4,5,6,7-tetrahydro-1H-pyrazolo[4,3-c]pyridin-1-yl)piperidin-1-yl)ethyl)benzyl)oxy)-1-oxoisoindolin-2-yl)piperidine-2,6-dione (P-173); or
3-(4-(4-((4-(5-acetyl-3-(7-(difluoromethyl)-6-(1-methyl-1H-pyrazol-4-yl)-3,4-dihydroquinolin-1(2H)-yl)-4,5,6,7-tetrahydro-1H-pyrazolo[4,3-c]pyridin-1-yl)piperidin-1-yl)methyl)phenethoxy)-1-oxoisoindolin-2-yl)piperidine-2,6-dione (P-174).

70. A composition comprising a bivalent compound according to claim 1 or a pharmaceutically acceptable salt or analog thereof, and a pharmaceutically acceptable carrier or diluent.

71. A method of treating a CBP/P300-mediated disease, comprising administering to a subject with a CBP/P300-mediated disease a bivalent compound or a pharmaceutically acceptable salt or analog thereof according to claim 1.

72-77. (canceled)

78. The method of claim 70 wherein the CBP/P300-mediated disease is selected from the group consisting of acoustic neuroma, acute leukemia, acute lymphocytic leukemia, acute myelocytic leukemia, acute T-cell leukemia, basal cell carcinoma, bile duct carcinoma, bladder cancer, brain choriocarcinoma, chronic leukemia, chronic lymphocytic leukemia, chronic myelocytic leukemia, chronic myelogenous leukemia, colon cancer, colorectal cancer, craniopharyngioma, cystadenocarcinoma, diffuse large B-cell lymphoma, dysproliferative changes, embryonal carcinoma, endometrial cancer, endotheliosarcoma, ependymoma, epithelial carcinoma, erythroleukemia, esophageal cancer, estrogen-receptor positive breast cancer, essential thrombocythemia, Ewing's tumor, fibrosarcoma, follicular lymphoma, germ cell testicular cancer, glioma, glioblastoma, gliosarcoma, heavy chain disease, head and neck cancer, hemangioblastoma, hepatoma, hepatocellular cancer, hormone insensitive prostate cancer, leiomyosarcoma, leukemia, liposarcoma, lung cancer, lymphagioendotheliosarcoma, lymphangiosarcoma, lymphoblastic leukemia, lymphoma, lymphoid malignancies of T-cell or B-cell origin, medullary carcinoma, medulloblastoma, melanoma, meningioma, mesothelioma, multiple myeloma, myelogenous leukemia, myeloma, myxosarcoma, neuroblastoma, NUT midline carcinoma (NMC), non-small cell lung cancer, oligodendroglioma, oral cancer, osteogenic sarcoma, ovarian cancer, pancreatic cancer, papillary adenocarcinomas, papillary carcinoma, pinealoma, polycythemia vera, prostate cancer, rectal cancer, renal cell carcinoma, retinoblastoma, rhabdomyosarcoma, sarcoma, sebaceous gland carcinoma, seminoma, skin cancer, small cell lung carcinoma, solid tumors (carcinomas and sarcomas), small cell lung cancer, stomach cancer, squamous cell carcinoma, synovioma, sweat gland carcinoma, thyroid cancer, Waldenstrom's macroglobulinemia, testicular tumors, uterine cancer, Wilms' tumor, prostate cancer, lung cancer, breast cancer, pancreatic cancer, colorectal cancer, melanoma, Addison's disease, acute gout, ankylosing spondylitis, asthma, atherosclerosis, Behcet's disease, bullous skin diseases, chronic obstructive pulmonary disease, Crohn's disease, dermatitis, eczema, giant cell arteritis, fibrosis, glomerulonephritis, hepatic vascular occlusion, hepatitis, hypophysitis, immunodeficiency syndrome, inflammatory bowel disease, Kawasaki disease, lupus nephritis, multiple sclerosis, myocarditis, myositis, nephritis, organ transplant rejection, osteoarthritis, pancreatitis, pericarditis, Polyarteritis nodosa, pneumonitis, primary biliary cirrhosis, psoriasis, psoriatic arthritis, rheumatoid arthritis, scleritis, sclerosing cholangitis, sepsis, systemic lupus erythematosus, Takayasu's Arteritis, toxic shock, thyroiditis, type I diabetes, ulcerative colitis, uveitis, vitiligo, vasculitis, and Wegener's granulomatosis.

79-85. (canceled)
